(19) United States
(12) Patent Application Publication
(10) Pub. No.: US 2024/0208992 A1
(43) Pub. Date: Jun. 27, 2024

(54) MACROCYCLES AS CFTR MODULATORS

(71) Applicant: Idorsia Pharmaceuticals Ltd, Allschwil (CH)

(72) Inventors: Martin BOLLI, Allschwil (CH); Christine BROTSCHI, Allschwil (CH); Malgorzata COMMANDEUR, Nijmegen BB (NL); John GATFIELD, Allschwil (CH); Thierry KIMMERLIN, Allschwil (CH); Hervé SIENDT, Allschwil (CH); Jasper SPRINGER, Diepenveen ZN (NL); Clemens WAGNER, Nijmegen BB (NL); Anita WEGERT, Nijmegen BB (NL); Jodi T. WILLIAMS, Allschwil (CH)

(21) Appl. No.: 18/550,883

(22) PCT Filed: Jul. 12, 2021
(86) PCT No.: PCT/EP2021/069292

§ 371 Date: Sep. 15, 2023

(30) Foreign Application Priority Data
Mar. 16, 2021 (WO) PCT/EP2021/056724

(51) Int. Cl. C07D 498/04; A61K 31/4745

(52) U.S. Cl. CPC C07D 498/04; A61K 31/4745

(57) ABSTRACT

The present invention relates to macrocyclic compounds of Formula (I)

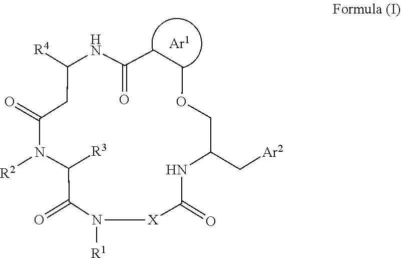

wherein Ar¹, Ar², R¹, R², R³, R⁴, and X are as described in the description, their preparation, to pharmaceutically acceptable salts thereof, and to their use as pharmaceuticals, to pharmaceutical compositions containing one or more compounds of Formula (I), and especially to their use as modulators of CFTR.

The present invention relates to novel macrocyclic compounds of formula (I) and their use as pharmaceuticals in the treatment of CFTR-related diseases and disorders such as especially cystic fibrosis. The invention also concerns related aspects including processes for the preparation of the compounds, pharmaceutical compositions containing one or more compounds of formula (I), and their use as modulators of CFTR.

Cystic Fibrosis (CF; mucoviscidosis, sometimes also called fibrocystic disease of pancreas or pancreatic fibrosis) is an autosomal recessive genetic disease caused by a dysfunctional epithelial chloride/bicarbonate channel named Cystic Fibrosis Transmembrane Conductance Regulator (CFTR). CFTR dysfunction leads to dysregulated chloride, bicarbonate and water transport at the surface of secretory epithelia causing accumulation of sticky mucus in organs including lung, pancreas, liver and intestine and, as a consequence, multi-organ dysfunction. Most debilitating effects in CF are nowadays observed in the lung which—due to abnormal hydration of airway surface liquid, mucus plugging, impaired mucociliary clearance, chronic inflammation and infection—loses its functionality over time leading to death by respiratory failure (Elborn, 2016). Human CFTR is a multidomain protein of 1480 amino acids. Many different mutations causing CFTR dysfunction have been discovered in CF patients leading e.g. to no functional CFTR proteins (class I mutations), CFTR trafficking defects (class II mutations), CFTR regulation defects (also known as gating defects; class Ill mutations), CFTR conductance defects (class IV mutations), less CFTR protein either due to splicing defects (class V mutations) or due to reduced CFTR stability (class VI mutations), no CFTR protein due to mRNA instability (class VII mutations) (de Boeck, Acta Paediatr. 2020, 109(5):893-895). The CFTR2 database (http://cftr2.org; data retrieved 06.07.2021) currently contains information on 360 disease-causing mutations. By far the most common disease-causing mutation is the deletion of phenylalanine at position 508 (F508del; allele frequency 0.697 in the CFTR2 database), that leads to misfolding of the channel during synthesis at the endoplasmic reticulum, degradation of the misfolded protein and a resulting strongly reduced transport to the cell surface (class II mutation). The residual F508del-CFTR that is trafficked to the cell surface is functional, however less than wildtype CFTR, i.e. F508del-CFTR also harbours a gating defect (Dalemans, 1991). Ca 40% of all CF patients are homozygous for the F508del mutation while another ˜40% of patients are heterozygous for the F508del mutation and carry another disease-causing mutation from class 1, 11, 111, IV, V, VI or VII. Such disease-causing mutations are considerably rarer with the class III G551D mutation (allele frequency 0.0210) and the class I G542X mutation (allele frequency 0.0254) and the class II N1303K mutation (allele frequency 0.0158) being the next most prevalent CF is currently treated by a range of drugs addressing the various organ symptoms and dysfunctions. Intestinal and pancreatic dysfunction are treated from diagnosis by food supplementation with pancreatic digestive enzymes. Lung symptoms are mainly treated with hypertonic saline inhalation, mucolytics, anti-inflammatory drugs, bronchiodilators and antibiotics (Elborn, 2016).

In addition to symptomatic treatments, CFTR modulators have been developed and approved for patients with certain CFTR mutations. These compounds directly improve CFTR trafficking to the cell surface (CFTR correctors) or improve CFTR function at the cell surface (CFTR potentiators). CFTR modulators can also enhance function of non-mutated (i.e. wildtype) CFTR and are therefore being studied in disorders where increasing wildtype CFTR function would have beneficial effects in non-CF disorders such as chronic bronchitis/COPD (Le Grand, J Med Chem. 2021, 64(11):7241-7260. Patel, Eur Respir Rev. 2020, 29(156):190068) and dry eye disease (Flores, FASEB J. 2016, 30(5):1789-1797).

CFTR modulators and their combinations can be discovered and optimized by assessing their ability to promote trafficking and function of mutated CFTR in in vitro cultivated recombinant and primary cellular systems. Activity in such systems is predictive of activity in CF patients.

WO2019/161078 discloses macrocycles as modulators of cystic fibrosis, wherein said macrocycles generally are 15-membered macrocycles comprising a (pyridine-carbonyl)-sulfamoyl moiety that is linked to a further aromatic group. Macrocyclic tetrapeptides (12- or 13-membered) including the compound Apicidin (CAS: 183506-66-3) have been proposed as potential agents for treating CF (Hutt D M et al. ACS Med Chem Lett. 2011; 2(9):703-707. doi:10.1021/m1200136e). WO2020/128925 discloses macrocycles capable of modulating the activity of CFTR, wherein said macrocycles comprise an optionally substituted divalent N-(pyridine-2-yl)pyridinyl-sulfonamide moiety. Non macrocyclic CFTR correctors and/or potentiators of CFTR have been disclosed for example in WO2011/119984, WO2014/015841, WO2007/134279, WO2010/019239, WO2011/019413, WO2012/027731, WO2013/130669, WO2014/078842 and WO2018/227049, WO2010/037066, WO2011/127241, WO2013/112804, WO2014/071122, and WO2020/128768. Furthermore, particular macrocycles can be found as screening compounds, wherein the phenylene group which is part of said macrocycles is always unsubstituted (CAS registry number: CAS-2213100-89-9, CAS-2213100-96-8, CAS-2213100-99-1, CAS-2213101-02-9, CAS-2213101-04-1, CAS-2213101-06-3, CAS-2213101-08-5, CAS-2213101-09-6, CAS-2213101-19-8, CAS-2213101-24-5, CAS-2215788-95-5, CAS-2215788-98-8, CAS-2215789-01-6, CAS-2215789-02-7, CAS-2215789-09-4, CAS-2215789-15-2, CAS-2215789-20-9, CAS-2215789-24-3, CAS-2215789-35-6, CAS-2215789-37-8, CAS-2215946-94-2, CAS-2215947-04-7, CAS-2215947-13-8, CAS-2215947-24-1, CAS-2215947-34-3, CAS-2215947-44-5, CAS-2215947-51-4, CAS-2215947-64-9, CAS-2215947-68-3, CAS-2215947-78-5, CAS-2215947-91-2, CAS-2215954-57-5, CAS-2216342-34-4, CAS-2216342-78-6, CAS-2216342-86-6, CAS-2216343-03-0, CAS-2216343-09-6, CAS-2216343-14-3, CAS-2216343-18-7, CAS-2216343-24-5, CAS-2216343-32-5, CAS-2216343-38-1, CAS-2216343-45-0, CAS-2216343-53-0, CAS-2216343-59-6, CAS-2216343-64-3, CAS-2216343-74-5, CAS-2216343-76-7).

The present invention provides macrocyclic compounds which are modulators of CFTR. The present compounds may, thus, be useful for the treatment of cystic fibrosis.
1) A first aspect of the invention relates to compounds of the Formula (I)

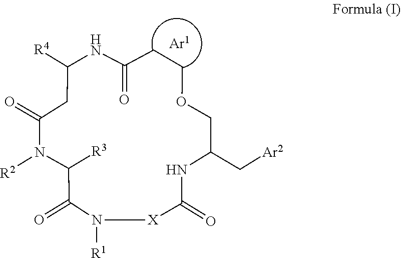

wherein
   X represents—CR^{X1}R^{X2}, wherein
      R^{X1} and R^{X2} together with the carbon atom to which they are attached form a ring which is:
         C₃₋₆-cycloalkan-1,1-diyl (especially cyclopropan-1,1-diyl, cyclobutan-1,1-diyl, cyclopentane-1,1diyl);
         C₅₋₆-cycloalkan-1,1-diyl which is fused to a benzene ring (especially 1,3-dihydro-2H-indene-2,2-diyl);
         C₃₋₆-cycloalkan-1,1-diyl, wherein said C₃₋₆-cycloalkan-1,1-diyl group independently is mono-substituted with C₁₋₃-alkoxy, fluoro, or hydroxy; or di-substituted with fluoro;
         C₄₋₆-heterocycloalkan-diyl, wherein said C₄₋₆-heterocycloalkan-diyl contains one ring nitrogen atom, wherein said nitrogen when having a free valency is unsubstituted or mono-substituted wherein the substitutents are independently selected from C₁₋₄-alkyl, and —COO—C₁₋₃-alkyl; or
         C₄₋₆-heterocycloalkan-diyl, wherein said C₄₋₆-heterocycloalkan-diyl contains one ring oxygen atom;
      R^{X1} and R^{X2} both independently represent C₁₋₄-alkyl (especially such group X is propan-2,2-diyl); or
      R^{X1} represents hydrogen, and
      R^{X2} represents
         hydrogen;
         C₁₋₆-alkyl (especially C₁₋₄-alkyl);
         C₁₋₄-fluoroalkyl;
         C₃₋₆-cycloalkyl;
         C₁₋₃-alkyl wherein said C₁₋₃-alkyl is mono-substituted with
         hydroxy;
         C₁₋₄-alkoxy;
         -L^{X1}-C₃₋₆-cycloalkyl wherein said C₃₋₆-cycloalkyl is unsubstituted or di-substituted with fluoro; and wherein L^{X1} independently represents a direct bond or oxygen;
         C₄₋₆-heterocycloalkyl wherein said C₄₋₆-heterocycloalkyl contains one ring oxygen atom;
         -NR^{N1}R^{N2} wherein R^{N1} and R^{N2} together with the nitrogen form a 4- to 6-membered carbocyclic ring comprising the nitrogen atom (i.e. an azetidin-1-yl, pyrrolidin-1-yl or piperidin-1-yl ring), wherein said ring is mono- or di-substituted with fluoro;
         a partially aromatic bicyclic ring, which is

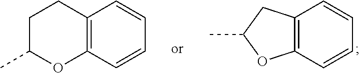

or
-L^{X2}-Ar^{X2} wherein—
L^{X2} independently represents a direct bond, C₁₋₃-alkylene, —C₁₋₃-alkylene-O—*, or —C₁₋₃-alkylene-O—C₁₋₂-alkylene*; wherein the asterisks indicate the bond that is attached to the group Ar^{X2}; and
ArX² independently represents aryl (especially phenyl, or napthyl), or 5- to 10-membered heteroaryl (especially oxadiazolyl, triazolyl, isoxazolyl, pyridinyl, or quinolinyl); wherein said group Ar^{X2}independently is unsubstituted, or mono-, or di-substituted wherein the substituents are independently selected from
C₁₋₄-alkyl;
C₁₋₃-alkoxy;
halogen;
C₃₋₆-cycloalkyl;
C₁₋₃-fluoroalkyl; and
Ar^{X3} wherein Ar^{X3} independently represents phenyl, or 5- or 6-membered heteroaryl (especially pyridinyl); wherein said group Ar^{X3} independently is unsubstituted, or mono-, or di-substituted wherein the substituents independently are selected from C₁₋₃-alkyl, C₁₋₃-alkoxy, C₁₋₃-alkoxy-C₂₋₃-alkyl, C₃₋₅-cycloalkyl, C₁₋₃-fluoroalkyl, and halogen; and
R¹ independently represents
   hydrogen;
   —C₁₋₆-alkyl (especially methyl);
   —C₂₋₆-alkyl wherein said C₂₋₆-alkyl is mono-substituted with hydroxy, or C₁₋₄-alkoxy (especially methoxy, tert-butoxy);
   —C₁₋₆-alkyl (wherein especially said —C₁₋₆-alkyl is —(CH₂)ₘ— wherein m represents the integer 1 or 2) wherein said C₁₋₆-alkyl is mono-substituted with R¹¹; wherein R¹¹ independently represents
      a saturated 5- or 6-membered heterocycloalkyl containing one or two ring heteroatoms wherein said heteroatoms are independently selected from nitrogen and oxygen, wherein said 5- or 6-membered heterocycloalkyl is independently unsubstituted, mono- or di-substituted, wherein the substituents independently are selected from C₁₋₄-alkyl (especially methyl), halogen, and benzyl;
      C₃₋₆-cycloalkyl (especially cyclohexyl), wherein said C₃₋₆-cycloalkyl is unsubstituted or mono-substituted with C₁₋₄-alkoxy (especially methoxy);
      phenyl or 5- or 6-membered heteroaryl (especially pyridinyl), wherein said phenyl or 5- or 6-membered heteroaryl independently is is unsubstituted, mono- or di-substituted wherein the substitutents independently are selected from C₁₋₄-alkyl, C₁₋₄-alkoxy, C₁₋₃-fluoroalkyl, C₁₋₃-fluoroalkoxy, halogen, cyano, and morpholin-4-yl;
      benzyloxy;
      a spirocyclic fragment, which is

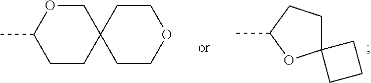

a saturated bicyclic ring, which is

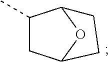

or
a partially aromatic bicyclic ring, which is

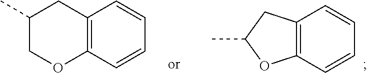

or the fragment

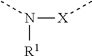

represents a heterocyclic ring which is

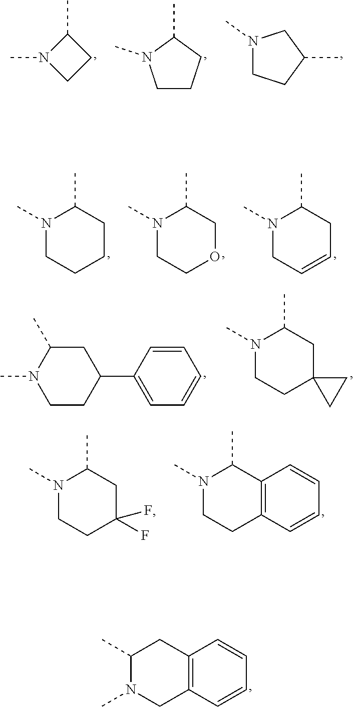

or, in addition,

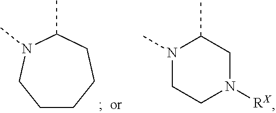

wherein R^{X} represents
hydrogen;
C₁₋₄-alkyl;
C₃₋₆-cycloalkyl;
C₁₋₄-alkyl, wherein said C₁₋₄-alkyl is mono-substituted with C₃₋₄-cycloalkyl;
C₂₋₄-alkyl, wherein said C₂₋₄-alkyl is mono-substituted with hydroxy or C₁₋₃-alkoxy;
phenyl or 5- or 6-membered heteroaryl (especially pyridinyl), wherein said phenyl or 5- or 6-membered heteroaryl independently is unsubstituted, mono- or di-substituted wherein the substituents independently are selected from C₁₋₄-alkyl, C₁₋₃-alkoxy, C₁₋₃-fluoroalkyl, C₁₋₃-fluoroalkoxy, cyano or halogen [especially such group is phenyl or pyridinyl; wherein said group is independently unsubstituted or substituted as defined before];

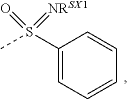

wherein R^{SX1} represents hydrogen or —CO—O—C₁₋₄-alkyl;
—CO—R^{OX1}, or —SO₂—R^{OX1}; wherein R^{OX1} independently represents
C₁₋₄-alkyl;
   C₁₋₃-alkyl wherein said C₁₋₃-alkyl is mono-substituted with C₁₋₃-alkoxy (especially methoxy), tetrahydropyranyl, morpholin-4-yl, phenyl, 10-membered heteroaryl (especially quinolinyl), or —NR^{ONX1}R^{ONX2} wherein R^{ONX1} and R^{ONX2}independently represent hydrogen or C₁₋₃-alkyl;
   tetrahydropyranyl;
   phenyl or 5- or 6-membered heteroaryl (especially pyridinyl or pyrazinyl), wherein said phenyl or 5- or 6-membered heteroaryl independently is unsubstituted, mono- or di-substituted wherein the substituents independently are selected from C₁₋₄-alkyl, C₁₋₃-alkoxy, C₁₋₃-fluoroalkyl, C₁₋₃-fluoroalkoxy, cyano or halogen; or
   a group of the structure (R^{X-A}):

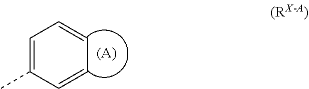

wherein (A) represents a non-aromatic 5- or 6-membered ring fused to the phenyl group, wherein ring (A) comprises two heteroatoms independently selected from oxygen and nitrogen; wherein said ring (A) independently is unsubstituted or mono-substituted wherein the substitutents independently are selected from oxo and C₁₋₃-alkyl; or
CO—O—R^{OX2}; wherein R^{OX2} represents
   C₁₋₄-alkyl;
   2,2,2-trichloroethyl; or
   tetrahydropyranyl;
R² represents C₁₋₄-alkyl (especially methyl);
R³ represents hydrogen; C₁₋₆-alkyl (especially methyl, isobutyl); —CH₂—C₃₋₆-cycloalkyl (especially —CH₂-cyclopropyl, —CH₂-cyclobutyl, —CH₂-cyclopentyl, —CH₂-cyclohexyl); or C₂₋₄-alkynyl (especially —CH₂—C≡CH);
R⁴ represents a group —CO—NH—R⁴¹; wherein R⁴¹ represents
   C₂₋₆-alkyl, which is mono-substituted with C₁₋₄-alkoxy (especially methoxy), C₁₋₄-fluoroalkoxy (especially difluoromethoxy, or trifluoromethoxy), or hydroxy;
   C₁₋₃-alkoxy-C₂₋₃-alkyleneO—CH₂—CH₂—;
   CH₂—CH₂—C₅₋₆-heterocycloalkyl, wherein said C₅₋₆-heterocycloalkyl contains one ring oxygen atom, wherein said C₅₋₆-heterocyclyl is unsubstituted, mono- or di-substituted with C₁₋₄-alkyl (especially methyl);
   -L¹-aryl; wherein L¹ represents —CH₂—CH₂—, —CH₂—CH₂—O—*, —CH₂—CF₂—*, —CH₂-(cyclopropan-1,1-diyl)-*, —CH(CH₂—OH)—CH₂—*, or —CH₂—CH(OH)—*; wherein asterisks indicate the bond with which L¹ is attached to the aryl; wherein aryl represents phenyl or naphthyl (especially phenyl); wherein said aryl is unsubstituted, mono-, di- or tri-substituted, wherein the substituents are independently C₁₋₄-alkyl (especially methyl, ethyl, tert-butyl), C₁₋₄-alkoxy (especially methoxy, ethoxy), C₁₋₃-fluoroalkyl, C₁₋₃-fluoroalkoxy, halogen (especially fluoro, chloro, bromo), cyano, C₃₋₆-cycloalkyl, C₃₋₆-cycloalkyl-methyl, C₁₋₃-alkoxy-C₁₋₃-alkyl, hydroxy-C₁₋₃-alkyl, C₂₋₃-alkynyl, morpholin-4-yl, C₁₋₃-alkyl-SO₂—, 5- or 6-membered heteroaryl (especially pyrazolyl, triazolyl, pyrimidinyl, pyrazinyl), or —NR^{N41}R^{N42}, wherein independently R^{N41} is hydrogen or C₁₋₄-alkyl, and R^{N42} is hydrogen or C₁₋₄-alkyl;
   -L²-HET; wherein L² represents —CH₂—CH₂—, —CH₂—CH₂—O—*, —CH₂—CF₂—*, —CH₂-(cyclopropan-1,1-diyl)-*, —CH(CH₂—OH)—CH₂—*, or —CH₂—CH(OH)—*; wherein asterisks indicate the bond with which L² is attached to HET (especially L² represents —CH₂—CH₂—); wherein HET represents 5- to 10-membered heteroaryl, wherein said HET is independently unsubstituted, mono-, di- or tri-substituted, wherein the substituents are independently C₁₋₄-alkyl (especially methyl, ethyl, isopropyl, tert-butyl); C₁₋₄-alkoxy (especially methoxy); C₁₋₃-fluoroalkyl; C₁₋₃-fluoroalkoxy; halogen; cyano; C₃₋₆-cycloalkyl; C₃₋₆-cycloalkyl-methyl; C₁₋₃-alkoxy-C₁₋₃-alkyl; hydroxy-C₁₋₃-alkyl; C₂₋₃-alkynyl; benzyl; or phenyl which is unsubstituted, mono- or di-substituted wherein the substituents independently are C₁₋₄-alkyl (especially methyl), C₁₋₄-alkoxy (especially methoxy), or C₁₋₄-fluoroalkoxy (especially trifluoromethoxy);
   CH₂—CH₂—HCy¹, wherein HCy¹ represents a partially aromatic bicyclic ring system consisting of a phenyl ring which is fused to a 5- to 7-membered saturated heterocyclic ring containing one or two heteroatoms independently selected from oxygen and nitrogen, wherein, if present, said nitrogen when having a free valency is unsubstituted or mono-substituted with C₁₋₄-alkyl (especially methyl); and wherein the phenyl ring of said partially aromatic bicyclic ring system is unsubstituted, mono-, di- or tri-substituted, wherein the substituents are independently C₁₋₄-alkyl (especially methyl), C₁₋₄-alkoxy (especially methoxy), C₁₋₃-fluoroalkyl, C₁₋₃-fluoroalkoxy, halogen (especially chloro), or cyano;
   —CH₂—CH₂—HCy², wherein HCy² represents a partially aromatic bicyclic ring system consisting of a 5-membered heteroaryl which is fused to a 5- to 7-membered saturated carbocyclic ring; or
   -L³-HCy³, wherein L³ represents a direct bond, or —CH₂—; wherein HCy³ represents a partially aromatic bicyclic ring system consisting of a phenyl ring which is fused to a 5- to 7-membered saturated heterocyclic ring containing one oxygen atom; wherein L³ is attached to said group HCy³ at a carbon atom which is part of said 5- to 7-membered saturated heterocyclic ring; and wherein the phenyl ring of said partially aromatic bicyclic ring system is unsubstituted, or mono-substituted with C₁₋₄-alkyl (especially methyl), C₁₋₄-alkoxy (especially methoxy), C₁₋₃-fluoroalkyl, C₁₋₃-fluoroalkoxy, halogen (especially chloro), or cyano;
Ar¹ represents
   5- or 6-membered heteroarylene wherein said 5- or 6-membered heteroarylene is unsubstituted (especially pyridin-3,4-diyl, thiophen-2,3-diyl);
phenylene, or 5- or 6-membered heteroarylene; wherein said phenylene, or 5- or 6-membered heteroarylene independently is mono-, di- or tri-substituted, wherein the substituents are independently selected from C₁₋₄-alkyl (especially methyl), C₁₋₄-alkoxy (especially methoxy, ethoxy), C₁₋₃-fluoroalkyl, C₁₋₃-fluoroalkoxy, cyano, and halogen (especially fluoro, chloro);
   phenylene wherein said phenylene is fused to a 5- or 6-membered saturated heterocyclic ring containing one or two oxygen atoms, wherein said 5- or 6-membered saturated heterocyclic ring independently is unsubstituted or di-substituted with fluoro; or
   a bicyclic aromatic ring selected from naphthylene and 8- to 10-membered bicyclic heteroarylene; wherein said bicyclic aromatic ring independently is unsubstituted, mono-, or di-substituted, wherein the substituents are independently selected from C₁₋₄-alkyl (especially methyl), C₁₋₃-fluoroalkyl, C₁₋₄-alkoxy, C₁₋₃-fluoroalkoxy, cyano, and halogen (especially fluoro, chloro); or
   quinoline-diyl, wherein said quinoline-diyl is present in form of the respective N-oxide; wherein said quinoline-diyl N-oxide is unsubstituted, or said quinoline-diyl N-oxide is mono-substituted with methyl or fluoro;
[wherein it is understood that in the above groups Ar¹ the —CO— group and the oxygen (i.e. the groups linking Ar¹ to the rest of the molecule) are attached in ortho arrangement to aromatic ring carbon atoms of Ar¹ as depicted in Formula (I)]; and
Ar² represents
   phenyl or naphthyl (especially phenyl), wherein said phenyl or naphthyl independently is unsubstituted, mono- or di-substituted wherein the substituents are independently selected from C₁₋₄-alkyl, C₁₋₃-fluoroalkyl, halogen, cyano, C₁₋₆-alkoxy, and C₁₋₃-fluoroalkoxy;
   5- or 6-membered heteroaryl (especially pyridinyl), wherein said 5- or 6-membered heteroaryl independently is unsubstituted or mono-substituted wherein the substituents are independently selected from C₁₋₄-alkyl, C₁₋₃-fluoroalkyl, halogen, cyano, C₁₋₆-alkoxy, and C₁₋₃-fluoroalkoxy; or
   9- or 10-membered heteroaryl (especially benzothiophenyl).
2) A further embodiment relates to compounds of the Formula (I) according to embodiment 1), wherein
   X represents —CR^{X1}R^{X2}, wherein
      R^{X1} and R^{X2} together with the carbon atom to which they are attached form a ring which is:
         C₃₋₆-cycloalkan-1,1-diyl- (especially cyclopropan-1,1-diyl, cyclobutan-1,1-diyl, cyclopentane-1,1-diyl);
         C₅₋₆-cycloalkan-1,1-diyl- which is fused to a benzene ring (especially 1,3-dihydro-2H-indene-2,2-diyl);
         C₃₋₆-cycloalkan-1,1-diyl-, wherein said C₃₋₆-cycloalkan-1,1-diyl group is mono-substituted with C₁₋₃-alkoxy, or di-substituted with fluoro (especially 3-methoxy-cyclobutan-1,1-diyl, 3,3-difluoro-cyclobutan-1,1-diyl); or
      R^{X1} and R^{X2} both independently represent C₁₋₄-alkyl (especially such group X is propan-2,2-diyl); or
      R^{X1} represents hydrogen, and
      R^{X2} represents
         hydrogen;
         C₁₋₆-alkyl (especially C₁₋₄-alkyl);
         C₁₋₄-fluoroalkyl;
         C₃₋₆-cycloalkyl;
         C₁₋₃-alkyl wherein said C₁₋₃-alkyl is mono-substituted with
         hydroxy;
         C₁₋₄-alkoxy;
         -L^{X1}-C₃₋₆-cycloalkyl wherein said C₃₋₆-cycloalkyl is unsubstituted or di-substituted with fluoro; and wherein L^{X1} independently represents a direct bond or oxygen;
         C₄₋₆-heterocycloalkyl wherein said C₄₋₆-heterocycloalkyl contains one ring oxygen atom;
         -NR^{N1}R^{N2} wherein R^{N1} and R^{N2} together with the nitrogen form a 4- to 6-membered carbocyclic ring comprising the nitrogen atom (i.e. an azetidin-1-yl, pyrrolidin-1-yl or piperidin-1-yl ring), wherein said ring is mono- or di-substituted with fluoro; or
         L^{X2}-Ar^{X2} wherein—
         L^{X2} independently represents a direct bond, C₁₋₃-alkylene, —C₁₋₃-alkylene-O—*, or —C₁₋₃-alkylene-O—C₁₋₂-alkylene*; wherein the asterisks indicate the bond that is attached to the group Ar^{X2}; and
         ArX² independently represents aryl (especially phenyl, or napthyl), or 5- to 10-membered heteroaryl (especially oxadiazolyl, triazolyl, isoxazolyl, pyridinyl, or quinolinyl); wherein said group Ar^{X2} independently is unsubstituted, or mono-, or di-substituted wherein the substituents are independently selected from
         C₁₋₄-alkyl;
         C₁₋₃-alkoxy;
         halogen;
         C₃₋₆-cycloalkyl;
         C₁₋₃-fluoroalkyl; and
         Ar^{X3} wherein Ar^{X3} independently represents phenyl, or 5- or 6-membered heteroaryl (especially pyridinyl); wherein said group Ar^{X3} independently is unsubstituted, or mono-, or di-substituted wherein the substituents independently are selected from C₁₋₃-alkyl, C₁₋₃-alkoxy, C₁₋₃-alkoxy-C₂₋₃-alkyl, C₃₋₅-cycloalkyl, C₁₋₃-fluoroalkyl, and halogen; and
R¹ independently represents
   hydrogen;
   —C₁₋₆-alkyl (especially methyl);
   —C₂₋₆-alkyl, wherein said C₂₋₆-alkyl is mono-substituted with C₁₋₄-alkoxy (especially methoxy, tert-butoxy);
   —C₃₋₆-alkyl, wherein said C₃₋₆-alkyl is mono-substituted with phenyl, or benzyloxy;
   —(CH₂)ₘ—R¹¹ wherein
      m represents the integer 1 or 2; and
      R¹¹ independently represents
         a saturated 5- or 6-membered heterocycloalkyl containing one or two ring oxygen atoms, wherein said 5- or 6-membered heterocycloalkyl is independently unsubstituted, mono- or di-substituted with C₁₋₄-alkyl (especially methyl);
         C₃₋₆-cycloalkyl (especially cyclobutyl, cyclohexyl), wherein said C₃₋₆-cycloalkyl is unsubstituted or mono-substituted with C₁₋₄-alkoxy (especially methoxy);
         phenyl or 5- or 6-membered heteroaryl (especially pyridinyl), wherein said phenyl or 5- or 6-membered heteroaryl independently is unsubstituted, mono- or di-substituted wherein the substituents independently are selected from C₁₋₄-alkyl, C₁₋₄-alkoxy, C₁₋₃-fluoroalkyl, C₁₋₃-fluoroalkoxy, cyano or halogen [especially such group represents phenyl];
         a spirocyclic fragment, which is

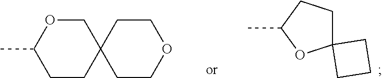

a saturated bicyclic ring, which is

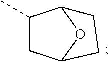

or
a partially aromatic bicyclic ring, which is

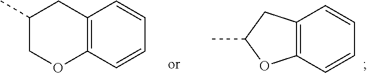

or the fragment

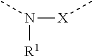

represents a heterocyclic ring which is

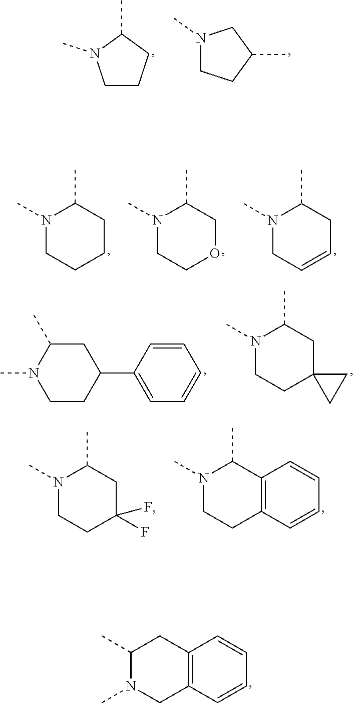

in addition,

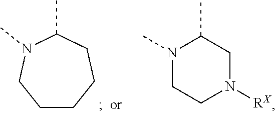

wherein R^{X} represents
   hydrogen;
   C₁₋₄-alkyl;
   C₃₋₆-cycloalkyl;
   C₁₋₄-alkyl, wherein said C₁₋₄-alkyl is mono-substituted with C₃₋₄-cycloalkyl;
   C₂₋₄-alkyl, wherein said C₂₋₄-alkyl is mono-substituted with hydroxy or C₁₋₃-alkoxy;
   phenyl or 5- or 6-membered heteroaryl (especially pyridinyl), wherein said phenyl or 5- or 6-membered heteroaryl independently is unsubstituted, mono- or di-substituted wherein the substituents independently are selected from C₁₋₄-alkyl, C₁₋₃-alkoxy, C₁₋₃-fluoroalkyl, C₁₋₃-fluoroalkoxy, cyano or halogen [especially such group is phenyl];

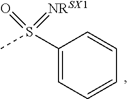

wherein R^{SX1} represents hydrogen or —CO—O—C₁₋₄-alkyl;
—CO—R^{OX1}, or —SO₂—R^{OX1}; wherein R^{OX1} independently represents
   C₁₋₄-alkyl;
   C₁₋₃-alkyl wherein said C₁₋₃-alkyl is mono-substituted with C₁₋₃-alkoxy (especially methoxy), tetrahydropyranyl, morpholin-4-yl, phenyl, 10-membered heteroaryl (especially quinolinyl), or —NR^{ONX1}R^{ONX2} wherein R^{ONX1} and R^{ONX2}independently represent hydrogen or C₁₋₃-alkyl;
   tetrahydropyranyl;
   phenyl or 5- or 6-membered heteroaryl (especially pyridinyl or pyrazinyl), wherein said phenyl or 5- or 6-membered heteroaryl independently is unsubstituted, mono- or di-substituted wherein the substituents independently are selected from C₁₋₄-alkyl, C₁₋₃-alkoxy, C₁₋₃-fluoroalkyl, C₁₋₃-fluoroalkoxy, cyano or halogen; or
   a group of the structure (R^{X-A}):

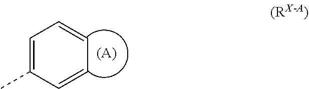

wherein (A) represents a non-aromatic 5- or 6-membered ring fused to the phenyl group, wherein ring (A) comprises two heteroatoms independently selected from oxygen and nitrogen; wherein said ring (A) independently is unsubstituted or mono-substituted wherein the substitutents independently are selected from oxo and C₁₋₃-alkyl; or
*—CO—O—R^{OX2}; wherein R^{OX2} represents
   C₁₋₄-alkyl;
   2,2,2-trichloroethyl; or
   tetrahydropyranyl;
R² represents C₁₋₄-alkyl (especially methyl);
R³ represents hydrogen; C₁₋₆-alkyl (especially methyl, isobutyl); —CH₂—C₃₋₆-cycloalkyl (especially —CH₂-cyclopropyl, —CH₂-cyclobutyl, —CH₂-cyclopentyl, —CH₂-cyclohexyl); C₂₋₄-alkynyl (especially —CH₂—C≡CH);
R⁴ represents a group —CO—NH—R⁴¹; wherein R⁴¹ represents
   C₂₋₆-alkyl, which is mono-substituted with C₁₋₄-alkoxy (especially methoxy), C₁₋₄-fluoroalkoxy (especially difluoromethoxy), or hydroxy;
   C₁₋₃-alkoxy-C₂₋₃-alkyleneO—CH₂—CH₂—;
   —CH₂—CH₂—C₅₋₆-heterocycloalkyl, wherein said C₅₋₆-heterocycloalkyl contains one ring oxygen atom, wherein said C₅₋₆-heterocyclyl is unsubstituted, mono- or di-substituted with C₁₋₄-alkyl (especially methyl);
   -L¹-aryl; wherein L¹ represents —CH₂—CH₂—, —CH₂—CH₂—O—*, —CH₂—CF₂—*, —CH₂-(cyclopropan-1,1-diyl)-*, —CH(CH₂—OH)—CH₂—*, or —CH₂—CH(OH)—*; wherein asterisks indicate the bond with which L¹ is attached to the aryl; wherein aryl represents phenyl or naphthyl (especially phenyl); wherein said aryl is unsubstituted, mono-, di- or tri-substituted, wherein the substituents are independently C₁₋₄-alkyl (especially methyl, ethyl, tert-butyl), C₁₋₄-alkoxy (especially methoxy, ethoxy), C₁₋₃-fluoroalkyl, C₁₋₃-fluoroalkoxy, halogen (especially fluoro, chloro, bromo), cyano, hydroxy-C₁₋₃-alkyl, C₂₋₃-alkynyl, morpholin-4-yl, C₁₋₃-alkyl-SO₂—, 5- or 6-membered heteroaryl (especially pyrazolyl, triazolyl, pyrimidinyl, pyrazinyl), or —NR^{N41}R^{N42}, wherein independently R^{N41} is hydrogen or C₁₋₄-alkyl, and R^{N42} is hydrogen or C₁₋₄-alkyl;
   -L²-HET¹; wherein L² represents —CH₂—CH₂—, —CH₂—CF₂—*, —CH₂-(cyclopropan-1,1-diyl)*, or —CH₂—CH(OH)—*; wherein asterisks indicate the bond with which L² is attached to HET (especially L² represents —CH₂—CH₂—); wherein HET¹ represents 5- or 6-membered heteroaryl (especially thiophenyl, furanyl, thiazolyl, isothiazolyl, pyrazolyl, isoxazolyl, oxadiazolyl, triazolyl, pyridinyl, pyrimidinyl, pyrazinyl), wherein said 5- or 6-membered heteroaryl is independently unsubstituted, mono-, di- or tri-substituted, wherein the substituents are independently C₁₋₄-alkyl (especially methyl); C₁₋₄-alkoxy (especially methoxy); C₁₋₃-fluoroalkyl; C₁₋₃-fluoroalkoxy; halogen; cyano; C₃₋₆-cycloalkyl (especially cyclopropyl); C₃₋₆-cycloalkyl-methyl; C₁₋₃-alkoxy-C₁₋₃-alkyl; C₂₋₃-alkynyl, benzyl; or phenyl which is unsubstituted, mono- or di-substituted wherein the substituents independently are C₁₋₄-alkyl (especially methyl), C₁₋₄-alkoxy (especially methoxy), or C₁₋₄-fluoroalkoxy (especially trifluoromethoxy);
   —CH₂—CH₂—HET², wherein HET² represents 9- or 10-membered bicyclic heteroaryl (especially benzoxazolyl, benzisoxazolyl, benzofuranyl, benzo[d][1,2,3]triazolyl or [1,2,4]triazolo[1,5-a]pyrimidinyl), wherein said HET² is unsubstituted or mono-substituted with C₁₋₄-alkyl;
   —CH₂—CH₂—HCy¹, wherein HCy¹ represents a partially aromatic bicyclic ring system consisting of a phenyl ring which is fused to a 5- to 7-membered saturated heterocyclic ring containing one or two heteroatoms independently selected from oxygen and nitrogen, wherein, if present, said nitrogen when having a free valency is unsubstituted or mono-substituted with C₁₋₄-alkyl (especially methyl); and wherein the phenyl ring of said partially aromatic bicyclic ring system is unsubstituted, mono-, di- or tri-substituted, wherein the substituents are independently C₁₋₄-alkyl (especially methyl), C₁₋₄-alkoxy (especially methoxy), C₁₋₃-fluoroalkyl, C₁₋₃-fluoroalkoxy, halogen (especially chloro), or cyano;
   —CH₂—CH₂—HCy², wherein HCy² represents a partially aromatic bicyclic ring system consisting of a 5-membered heteroaryl which is fused to a 5- to 7-membered saturated carbocyclic ring; or
HCy³; wherein HCy³ represents a partially aromatic bicyclic ring system consisting of a phenyl ring which is fused to a 5- to 7-membered saturated heterocyclic ring containing one oxygen atom; wherein said group HCy³ is bound to the nitrogen of the —CO—NH— group at a carbon atom which is part of said 5- to 7-membered saturated heterocyclic ring; and wherein the phenyl ring of said partially aromatic bicyclic ring system is unsubstituted, or mono-substituted with C₁₋₄-alkyl (especially methyl), or C₁₋₄-alkoxy (especially methoxy);
Ar¹ represents
   5- or 6-membered heteroarylene wherein said 5- or 6-membered heteroarylene is unsubstituted (especially pyridin-3,4-diyl, thiophen-2,3-diyl);
   phenylene, or 5- or 6-membered heteroarylene; wherein said phenylene, or 5- or 6-membered heteroarylene independently is mono-, di- or tri-substituted, wherein the substituents are independently selected from C₁₋₄-alkyl (especially methyl), C₁₋₄-alkoxy (especially methoxy, ethoxy), C₁₋₃-fluoroalkyl, C₁₋₃-fluoroalkoxy, cyano, and halogen (especially fluoro, chloro);
   phenylene wherein said phenylene is fused to a 5- or 6-membered saturated heterocyclic ring containing one or two oxygen atoms, wherein said 5- or 6-membered saturated heterocyclic ring independently is unsubstituted or di-substituted with fluoro; or
   a bicyclic aromatic ring selected from naphthylene and 8- to 10-membered bicyclic heteroarylene; wherein said bicyclic aromatic ring independently is unsubstituted, mono-, or di-substituted, wherein the substituents are independently selected from C₁₋₄-alkyl (especially methyl) and halogen (especially fluoro, chloro); or
   quinoline-diyl, wherein said quinoline-diyl is present in form of the respective N-oxide; wherein said quinoline-diyl N-oxide is unsubstituted, or said quinoline-diyl N-oxide is mono-substituted with methyl or fluoro;
   [wherein it is understood that in the above groups Ar¹ the —CO— group and the oxygen (i.e. the groups linking Ar¹ to the rest of the molecule) are attached in ortho arrangement to aromatic ring carbon atoms of Ar¹ as depicted in Formula (I)]; and
Ar² represents
   phenyl or naphthyl (especially phenyl);
   5- or 6-membered heteroaryl (especially pyridinyl); or
   9- or 10-membered heteroaryl (especially benzothiophenyl).
3) A second aspect relates to compounds of Formula (I) according to embodiment 1) or 2), wherein the compounds are compounds of Formula (I_{E}):

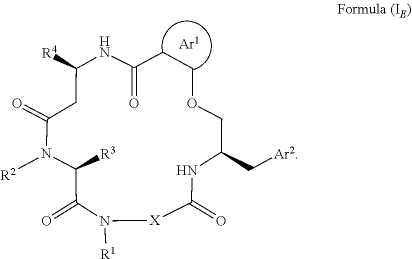

4) Another embodiment relates to compounds according to any one of embodiments 1) to 3), wherein
   X represents —CR^{X1}R^{X2}, wherein
      R^{X1} and R^{X2} together with the carbon atom to which they are attached form a ring which is:
         C₃₋₆-cycloalkan-1,1-diyl- (especially cyclopropan-1,1-diyl, cyclobutan-1,1-diyl, cyclopentane-1,1-diyl);
         C₅₋₆-cycloalkan-1,1-diyl- which is fused to a benzene ring (especially 1,3-dihydro-2H-indene-2,2-diyl); or
         C₃₋₆-cycloalkan-1,1-diyl-, wherein said C₃₋₆-cycloalkan-1,1-diyl group is mono-substituted with C₁₋₃-alkoxy, or di-substituted with fluoro (especially 3-methoxy-cyclobutan-1,1-diyl, 3,3-difluoro-cyclobutan-1,1-diyl);
      R^{X1} and R^{X2} both independently represent C₁₋₄-alkyl (especially such group X is propan-2,2-diyl); or
      R^{X1} represents hydrogen, and
      R^{X2} represents
         hydrogen;
         C₁₋₆-alkyl (especially C₁₋₄-alkyl);
         C₁₋₄-fluoroalkyl;
         C₃₋₆-cycloalkyl;
         C₁₋₃-alkyl wherein said C₁₋₃-alkyl is mono-substituted with
         hydroxy;
         C₁₋₄-alkoxy;
         -L^{X1}-C₃₋₆-cycloalkyl wherein said C₃₋₆-cycloalkyl is unsubstituted or di-substituted with fluoro; and wherein L^{X1} independently represents a direct bond or oxygen;
         C₄₋₆-heterocycloalkyl wherein said C₄₋₆-heterocycloalkyl contains one ring oxygen atom;
         -NR^{N1}R^{N2} wherein R^{N1} and R^{N2} together with the nitrogen form a 4- to 6-membered carbocyclic ring comprising the nitrogen atom (i.e. an azetidin-1-yl, pyrrolidin-1-yl or piperidin-1-yl ring), wherein said ring is mono- or di-substituted with fluoro;
         L^{X2}-Ar^{X2} wherein—
         L^{X2} independently represents a direct bond, C₁₋₃-alkylene, —C₁₋₃-alkylene-O—*, or —C₁₋₃-alkylene-O—C₁₋₂-alkylene-*; wherein the asterisks indicate the bond that is attached to the group Ar^{X2}; and
         ArX² independently represents aryl (especially phenyl, or napthyl), or 5- to 10-membered heteroaryl (especially oxadiazolyl, triazolyl, isoxazolyl, pyridinyl, or quinolinyl); wherein said group Ar^{X2} independently is unsubstituted, or mono-, or di-substituted wherein the substituents are independently selected from
         C₁₋₄-alkyl;
         C₁₋₃-alkoxy;
         halogen;
         cyano;
         C₃₋₆-cycloalkyl;
         C₁₋₃-fluoroalkyl; and
         Ar^{X3} wherein Ar^{X3} independently represents phenyl, or 5- or 6-membered heteroaryl (especially pyridinyl); wherein said group Ar^{X3} independently is unsubstituted, or mono-, or di-substituted wherein the substituents independently are selected from C₁₋₃-alkyl, C₁₋₃-alkoxy, C₁₋₃-alkoxy-C₂₋₃-alkyl, C₃₋₅-cycloalkyl, C₁₋₃-fluoroalkyl, and halogen; and
   R¹ independently represents hydrogen or —C₁₋₃-alkyl (especially methyl); or
   X represents —CR^{X1}R^{X2}, wherein
      R^{X1} represents hydrogen, and
      R^{X2} represents hydrogen, or methyl; (i.e. such group X representing —CR^{X1}R^{X2} wherein R^{X1} represents hydrogen is methylene, or ethan-1,1-diyl); or
      R^{X1} and R^{X2} together with the carbon atom to which they are attached form a ring which is C₃₋₅-cycloalkan-1,1-diyl- (especially cyclopropan-1,1-diyl); and
   R¹ independently represents
      C₄₋₆-alkyl (especially 3,3-dimethyl-butyl);
      C₂₋₆-alkyl, wherein said C₂₋₆-alkyl is mono-substituted with C₁₋₄-alkoxy (especially methoxy, tert-butoxy) (in particular such group is 2-methoxy-ethyl, 3-methoxy-propyl, 3-methoxy-3-methyl-butyl, 2-(tert-butoxy)-ethyl);
      C₃₋₆-alkyl, wherein said C₃₋₆-alkyl is mono-substituted with phenyl, or benzyloxy;
      (CH₂)ₘ—R¹¹ wherein m represents the integer 1 or 2; and R¹¹ independently represents
         a saturated 5- or 6-membered heterocycloalkyl containing one or two ring oxygen atoms (especially tetrahydrofuranyl, tetrahydropyranyl), wherein said 5- or 6-membered heterocycloalkyl is independently unsubstituted, mono- or di-substituted with C₁₋₄-alkyl (especially methyl);
         C₃₋₆-cycloalkyl (especially cyclobutyl, cyclohexyl), wherein said C₃₋₆-cycloalkyl is unsubstituted or mono-substituted with C₁₋₄-alkoxy (especially methoxy);
         phenyl or 5- or 6-membered heteroaryl (especially pyridinyl), wherein said phenyl or 5- or 6-membered heteroaryl independently is unsubstituted, mono- or di-substituted wherein the substituents independently are selected from C₁₋₄-alkyl, C₁₋₄-alkoxy, C₁₋₃-fluoroalkyl, C₁₋₃-fluoroalkoxy, cyano or halogen [especially such group represents phenyl];
         a spirocyclic fragment, which is

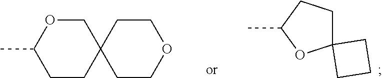

a saturated bicyclic ring, which is

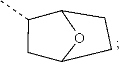

a partially aromatic bicyclic ring, which is

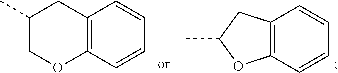

or the fragment

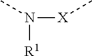

represents a heterocyclic ring which is

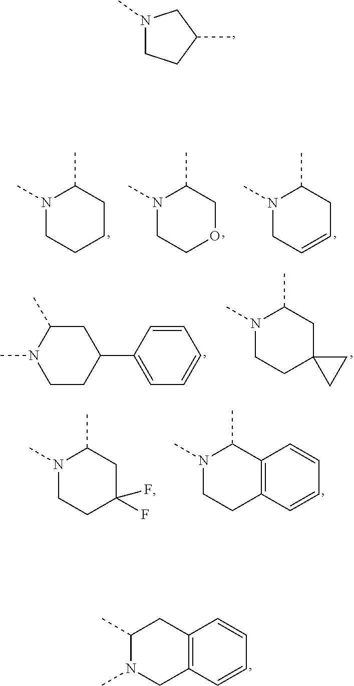

or, in addition,

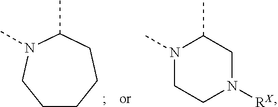

wherein R^{X} represents
   hydrogen;
   C₁₋₄-alkyl;
   C₃₋₄-cycloalkyl;
   C₁₋₄-alkyl, wherein said C₁₋₄-alkyl is mono-substituted with C₃₋₄-cycloalkyl;
   C₂₋₄-alkyl, wherein said C₂₋₄-alkyl is mono-substituted with hydroxy, or C₁₋₃-alkoxy;
   phenyl or 5- or 6-membered heteroaryl (especially pyridinyl), wherein said phenyl or 5- or 6-membered heteroaryl independently is unsubstituted, mono- or di-substituted wherein the substituents independently are selected from C₁₋₄-alkyl, C₁₋₃-alkoxy, C₁₋₃-fluoroalkyl, C₁₋₃-fluoroalkoxy, cyano or halogen [especially such group represents phenyl];

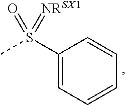

wherein R^{SX1} represents hydrogen or —CO—O—C₁₋₄-alkyl;
CO—R^{OX1}, or —SO₂—R^{OX1}; wherein R^{OX1} independently represents
   C₁₋₄-alkyl;
   C₁₋₃-alkyl wherein said C₁₋₃-alkyl is mono-substituted with C₁₋₃-alkoxy (especially methoxy), tetrahydropyranyl, morpholin-4-yl, phenyl, 10-membered heteroaryl (especially quinolinyl), or —NR^{ONX1}R^{ONX2} wherein R^{ONX1} and R^{ONX2}independently represent hydrogen or C₁₋₃-alkyl;
   tetrahydropyranyl;
   phenyl or 5- or 6-membered heteroaryl (especially pyridinyl or pyrazinyl), wherein said phenyl or 5- or 6-membered heteroaryl independently is unsubstituted, mono- or di-substituted wherein the substituents independently are selected from C₁₋₄-alkyl, C₁₋₃-alkoxy, C₁₋₃-fluoroalkyl, C₁₋₃-fluoroalkoxy, cyano or halogen; or
   a group of the structure (R^{X-A}):

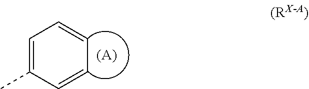

wherein (A) represents a non-aromatic 5- or 6-membered ring fused to the phenyl group, wherein ring (A) comprises two heteroatoms independently selected from oxygen and nitrogen; wherein said ring (A) independently is unsubstituted or mono-substituted wherein the substitutents independently are selected from oxo and C₁₋₃-alkyl; or
—CO—O—R^{OX2}; wherein R^{OX2} represents
   C₁₋₄-alkyl;
   2,2,2-trichloroethyl; or
   tetrahydropyranyl.
5) Another embodiment relates to compounds according to any one of embodiments 1) to 3), wherein
   X represents —CR^{X1}R^{X2}, wherein
      R^{X1} and R^{X2} together with the carbon atom to which they are attached form a ring which is:
         C₃₋₆-cycloalkan-1,1-diyl- (especially cyclopropan-1,1-diyl, cyclobutan-1,1-diyl, cyclopentane-1,1-diyl);
         C₅₋₆-cycloalkan-1,1-diyl- which is fused to a benzene ring (especially 1,3-dihydro-2H-indene-2,2-diyl);
         C₃₋₆-cycloalkan-1,1-diyl-, wherein said C₃₋₆-cycloalkan-1,1-diyl group is mono-substituted with C₁₋₃-alkoxy, or di-substituted with fluoro (especially 3-methoxy-cyclobutan-1,1-diyl, 3,3-difluoro-cyclobutan-1,1-diyl);
      R^{X1} and R^{X2} both independently represent C₁₋₄-alkyl (especially such group X is propan-2,2-diyl); or
      R^{X1} represents hydrogen, and
      R^{X2} represents
         hydrogen;
         C₁₋₆-alkyl (especially C₁₋₄-alkyl);
         C₁₋₄-fluoroalkyl;
         C₃₋₆-cycloalkyl;
         C₁₋₃-alkyl wherein said C₁₋₃-alkyl is mono-substituted with
         hydroxy;
         C₁₋₄-alkoxy;
         -L^{X1}-C₃₋₆-cycloalkyl wherein said C₃₋₆-cycloalkyl is unsubstituted or di-substituted with fluoro; and wherein L^{X1} independently represents a direct bond or oxygen;
         C₄₋₆-heterocycloalkyl wherein said C₄₋₆-heterocycloalkyl contains one ring oxygen atom;
         -NR^{N1}R^{N2} wherein R^{N1} and R^{N2} together with the nitrogen form a 4- to 6-membered carbocyclic ring comprising the nitrogen atom (i.e. an azetidin-1-yl, pyrrolidin-1-yl or piperidin-1-yl ring), wherein said ring is mono- or di-substituted with fluoro;
         a partially aromatic bicyclic ring, which is
         or

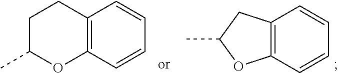

-L^{X2}-Ar^{X2} wherein—
L^{X2} independently represents a direct bond, C₁₋₃-alkylene, —C₁₋₃-alkylene-O—*, or —C₁₋₃-alkylene-O—C₁₋₂-alkylene-*; wherein the asterisks indicate the bond that is attached to the group Ar^{X2}; and Ar^{X2} represents phenyl, wherein said phenyl independently is unsubstituted, or mono-, or di-substituted wherein the substituents are independently selected from
C₁₋₄-alkyl;
C₁₋₃-alkoxy;
halogen;
C₃₋₆-cycloalkyl; and
C₁₋₃-fluoroalkyl; or
L^{X2} independently represents a direct bond, C₁₋₃-alkylene, —C₁₋₃-alkylene-O—*, or —C₁₋₃-alkylene-O—C₁₋₂-alkylene*; wherein the asterisks indicate the bond that is attached to the group Ar^{X2}; and Ar^{X2} independently represents 5- or 6-membered heteroaryl [notably 5-membered heteroaryl containing one to three heteroatoms independently selected from oxygen and nitrogen (especially oxadiazolyl, triazolyl, or isoxazolyl); or 6-membered heteroaryl containing one or two nitrogen atoms (especially pyridinyl or pyrazinyl)]; wherein said heteroaryl independently is unsubstituted, or mono-, or di-substituted wherein the substituents are independently selected from
C₁₋₄-alkyl;
C₁₋₃-alkoxy;
halogen;
C₃₋₆-cycloalkyl;
C₁₋₃-fluoroalkyl; and
Ar^{X3} wherein Ar^{X3} independently represents phenyl, or 5- or 6-membered heteroaryl (notably 6-membered heteroaryl containing one or two nitrogen atoms, especially pyridinyl); wherein said group Ar^{X3} independently is unsubstituted, or mono-, or di-substituted wherein the substituents independently are selected from C₁₋₃-alkyl, C₁₋₃-alkoxy, C₁₋₃-alkoxy-C₂₋₃-alkyl, C₃₋₅-cycloalkyl, C₁₋₃-fluoroalkyl, and halogen; and
L^{X2} independently represents a direct bond, C₁₋₃-alkylene, or —C₁₋₃-alkylene-O—*; wherein the asterisk indicates the bond that is attached to the group Ar^{X2}; and Ar^{X2}independently represents naphthyl or 8- to 10-membered heteroaryl (notably 10-membered heteroaryl containing one nitrogen atom, especially quinolinyl); wherein said group Ar^{X2}independently is unsubstituted, or mono-, or di-substituted wherein the substituents are independently selected from
C₁₋₄-alkyl;
C₁₋₃-alkoxy;
halogen;
C₃₋₆-cycloalkyl; and
C₁₋₃-fluoroalkyl;
and
R¹ independently represents —C₁₋₃-alkyl (especially methyl); or
X represents —CR^{X1}R^{X2}, wherein R^{X1} and R^{X2} both represent hydrogen; and
R¹ independently represents
   —C₄₋₆-alkyl;
   —C₂₋₆-alkyl, wherein said C₂₋₆-alkyl is mono-substituted with C₁₋₄-alkoxy (especially methoxy, tert-butoxy);
   —C₃₋₆-alkyl, wherein said C₃₋₆-alkyl is mono-substituted with phenyl, or benzyloxy;
   (CH₂)ₘ—R¹¹ wherein
   m represents the integer 1 or 2; and
   R¹¹ independently represents
      a saturated 5- or 6-membered heterocycloalkyl containing one or two ring oxygen atoms, wherein said 5- or 6-membered heterocycloalkyl is independently unsubstituted, mono- or di-substituted with C₁₋₄-alkyl (especially methyl);
      C₃₋₆-cycloalkyl (especially cyclobutyl, cyclohexyl), wherein said C₃₋₆-cycloalkyl is unsubstituted or mono-substituted with C₁₋₄-alkoxy (especially methoxy);
      phenyl;
      a spirocyclic fragment, which is

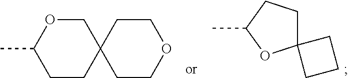

a saturated bicyclic ring, which is

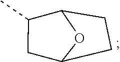

or
a partially aromatic bicyclic ring, which is

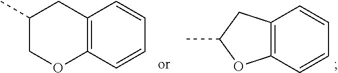

or the fragment

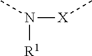

represents a heterocyclic ring which is

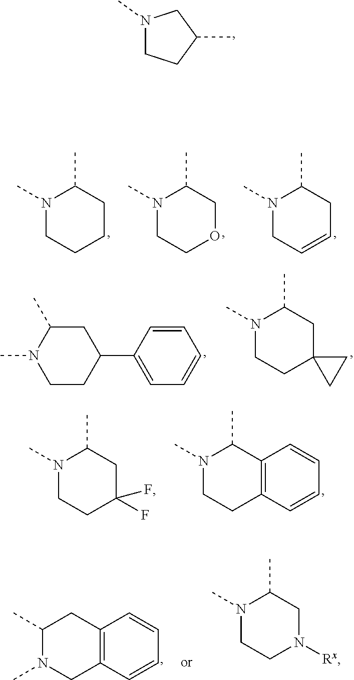

wherein R^{X} represents
   C₁₋₄-alkyl;
   C₃₋₄-cycloalkyl;
   C₁₋₄-alkyl, wherein said C₁₋₄-alkyl is mono-substituted with C₃₋₄-cycloalkyl;
   C₁₋₄-alkyl, wherein said C₁₋₄-alkyl is mono-substituted with hydroxy, or C₁₋₃-alkoxy;
   phenyl;
   6-membered heteroaryl (especially pyridinyl), wherein said 6-membered heteroaryl is unsubstituted or mono-substituted with halogen (especially fluoro);

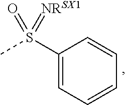

wherein R^{SX1} represents —CO—O—C₁₋₄-alkyl;
CO—R^{OX1}, or —SO₂—R^{OX1}; wherein R^{OX1} independently represents
   C₁₋₄-alkyl;
   C₁₋₃-alkyl wherein said C₁₋₃-alkyl is mono-substituted with C₁₋₃-alkoxy (especially methoxy), tetrahydropyranyl, morpholin-4-yl, phenyl, 10-membered heteroaryl (especially quinolinyl), or —NR^{ONX1}R^{ONX2} wherein R^{ONX1} and R^{ONX2}independently represent hydrogen or C₁₋₃-alkyl;
   tetrahydropyranyl;
   phenyl which is unsubstituted, mono- or di-substituted wherein the substituents independently are C₁₋₃-alkoxy, C₁₋₃-fluoroalkoxy or halogen;
   5- or 6-membered heteroaryl (especially pyridinyl, pyrazinyl) wherein said 5- or 6-membered heteroaryl is independently unsubstituted or mono-substituted with C₁₋₃-alkoxy; or
   a group of the structure (R^{X-A}):

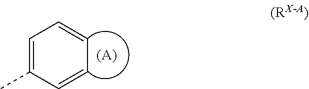

wherein (A) represents a non-aromatic 5- or 6-membered ring fused to the phenyl group,
wherein ring (A) comprises two heteroatoms independently selected from oxygen and nitrogen; wherein said ring (A) independently is unsubstituted or mono-substituted wherein the substitutents independently are selected from oxo and C₁₋₃-alkyl; or
CO—O—R^{OX2}; wherein R^{OX2} represents
   C₁₋₄-alkyl;
   2,2,2-trichloroethyl; or
   tetrahydropyranyl.
6) Another embodiment relates to compounds according to any one of embodiments 1) to 3), wherein the fragment

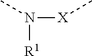

represents a group selected from:
A)

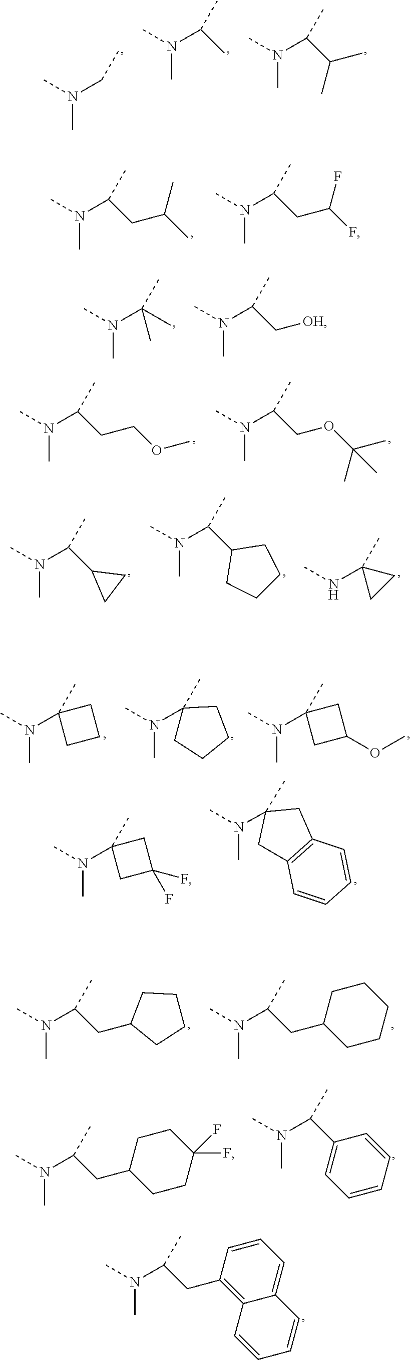

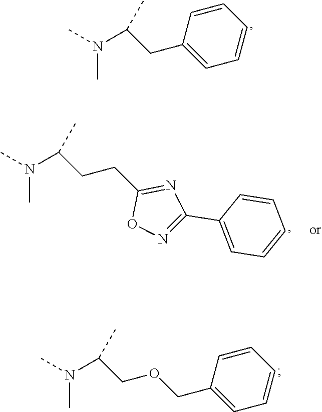

B)

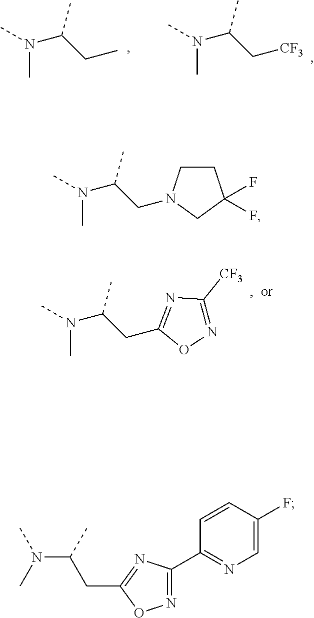

C)

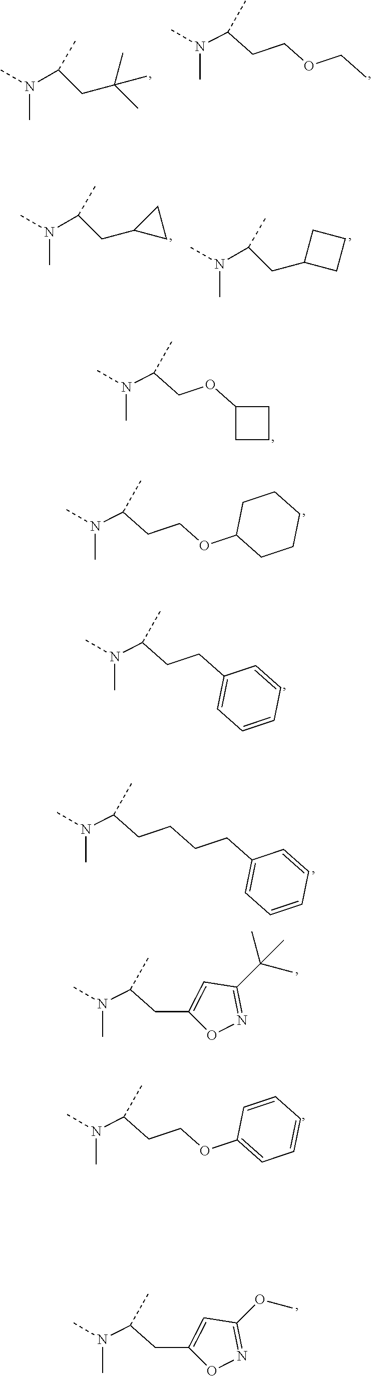

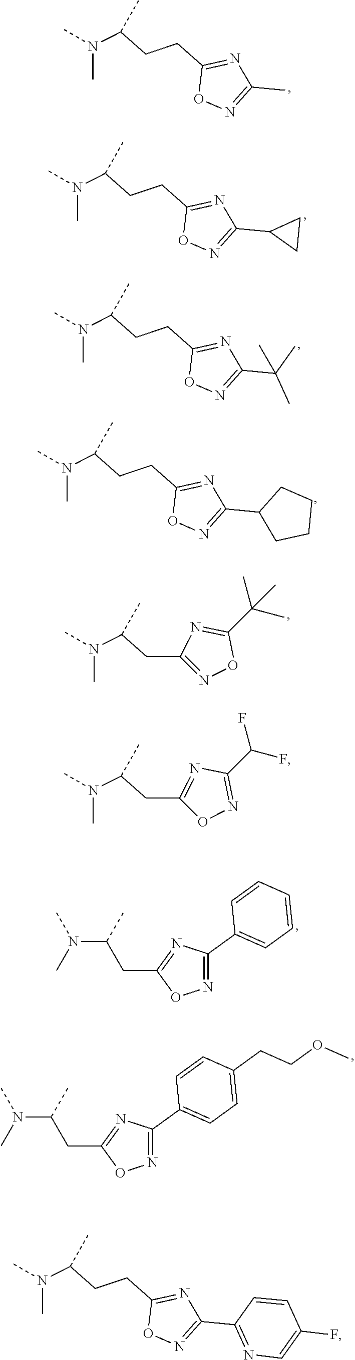

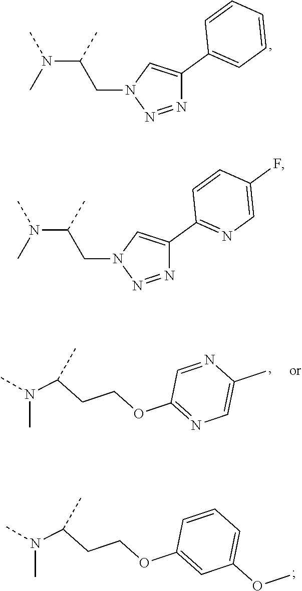

D)

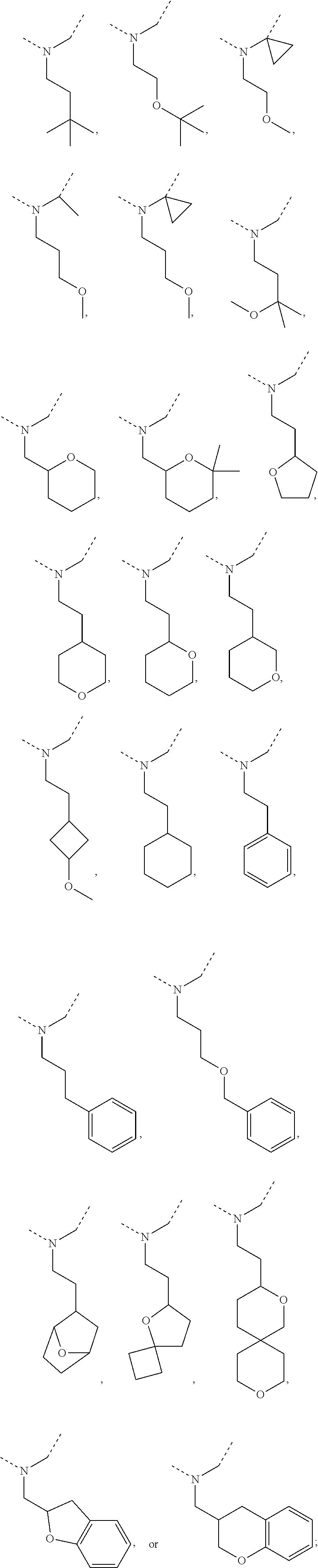

E)

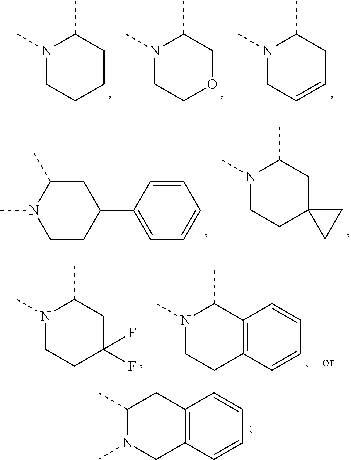

[especially

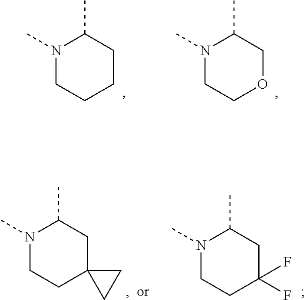

in particular

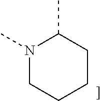

F)

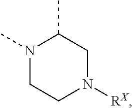

wherein R^{X} represents
   C₁₋₄-alkyl;
   C₃₋₄-cycloalkyl;
   C₁₋₄-alkyl, wherein said C₁₋₄-alkyl is mono-substituted with C₃₋₄-cycloalkyl;
   C₁₋₄-alkyl, wherein said C₁₋₄-alkyl is mono-substituted with hydroxy, or C₁₋₃-alkoxy;
   phenyl;
   6-membered heteroaryl (especially pyridinyl), wherein said 6-membered heteroaryl is unsubstituted or mono-substituted with halogen (especially fluoro);

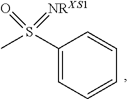

wherein R^{SX1} represents —CO—O—C₁₋₄-alkyl;
—CO—R^{OX1}, or —SO₂—R^{OX1}; wherein R^{OX1} independently represents
   C₁₋₄-alkyl;
   C₁₋₃-alkyl wherein said C₁₋₃-alkyl is mono-substituted with C₁₋₃-alkoxy (especially methoxy), tetrahydropyranyl, morpholin-4-yl, phenyl, 10-membered heteroaryl (especially quinolinyl), or —NR^{ONX1}R^{ONX2} wherein R^{ONX1} and R^{ONX2}independently represent hydrogen or C₁₋₃-alkyl;
   tetrahydropyranyl;
   phenyl which is unsubstituted, mono- or di-substituted wherein the substituents independently are C₁₋₃-alkoxy, C₁₋₃-fluoroalkoxy or halogen;
   5- or 6-membered heteroaryl (especially pyridinyl, pyrazinyl) wherein said 5- or 6-membered heteroaryl is independently unsubstituted or mono-substituted with C₁₋₃-alkoxy; or
   a group of the structure (R^{X-A}):

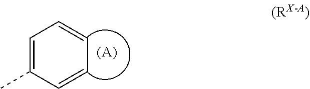

wherein (A) represents a non-aromatic 5- or 6-membered ring fused to the phenyl group, wherein ring (A) comprises two heteroatoms independently selected from oxygen and nitrogen; wherein said ring (A) independently is unsubstituted or mono-substituted wherein the substitutents independently are selected from oxo and C₁₋₃-alkyl; or
—CO—O—R^{OX2}; wherein R^{OX2} represents
   C₁₋₄-alkyl;
   2,2,2-trichloroethyl; or
   tetrahydropyranyl;
G)

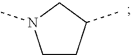

or H)

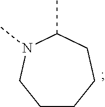

wherein the above groups A), B), C), D), E), F), G) and H) each form a particular sub-embodiment; wherein notably the groups A), B), and C) together form another sub-embodiment, the group D) forms another sub-embodiment, and the groups E), F), G), and H) together form another sub-embodiment. Another particular sub-embodiment is formed by the groups A), D), E), F) and G).
7) Another embodiment relates to compounds according to any one of embodiments 1) to 6), wherein R² represents methyl.
8) Another embodiment relates to compounds according to any one of embodiments 1) to 7), wherein R³ represents isobutyl.
9) Another embodiment relates to compounds according to any one of embodiments 1) to 8), wherein R⁴ represents a group —CO—NH—R⁴¹; wherein R⁴¹ represents
   C₂₋₆-alkyl, which is mono-substituted with C₁₋₄-alkoxy (especially methoxy), or C₁₋₄-fluoroalkoxy (especially difluoromethoxy, or trifluoromethoxy);
   C₁₋₃-alkoxy-C₂₋₃-alkyleneO—CH₂—CH₂—;
   —CH₂—CH₂—C₅₋₆-heterocycloalkyl, wherein said C₅₋₆-heterocycloalkyl contains one ring oxygen atom, wherein said C₅₋₆-heterocyclyl is unsubstituted, mono- or di-substituted with C₁₋₄-alkyl (especially methyl);
   -L¹-aryl; wherein L¹ represents —CH₂—CH₂—, —CH₂—CH₂—O—* or —CH₂—CF₂—*, —CH₂-(cyclopropan-1,1-diyl)-*, —CH(CH₂—OH)—CH₂—*, or —CH₂—CH(OH)—*; wherein asterisks indicate the bond with which L¹ is attached to the aryl; wherein aryl represents phenyl; wherein said aryl independently is unsubstituted, mono-, di- or tri-substituted, wherein the substituents are independently C₁₋₄-alkyl (especially methyl, ethyl, tert-butyl), C₁₋₄-alkoxy (especially methoxy, ethoxy), C₁₋₃-fluoroalkyl, C₁₋₃-fluoroalkoxy, halogen (especially fluoro, chloro, bromo), cyano, hydroxy-C₁₋₃-alkyl, C₂₋₃-alkynyl, morpholin-4-yl, C₁₋₃-alkyl-SO₂—, 5- or 6-membered heteroaryl (especially pyrazolyl, triazolyl, pyrimidinyl, pyrazinyl), or —NR^{N41}R^{N42}, wherein independently R^{N41} is hydrogen or C₁₋₄-alkyl, and R^{N42} is hydrogen or C₁₋₄-alkyl;
   -L²-HET¹; wherein L² represents —CH₂—CH₂—, —CH₂—CF₂—*, —CH₂-(cyclopropan-1,1-diyl)*, or —CH₂—CH(OH)—*; wherein asterisks indicate the bond with which L² is attached to HET¹ (especially L² represents —CH₂—CH₂—); wherein HET¹ represents 5- or 6-membered heteroaryl (especially thiophenyl, furanyl, thiazolyl, isothiazolyl, pyrazolyl, isoxazolyl, oxadiazolyl, or triazolyl; or pyridinyl, pyrimidinyl, or pyrazinyl), wherein said 5- or 6-membered heteroaryl is independently unsubstituted, mono-, or di-substituted, wherein the substituents are independently C₁₋₄-alkyl (especially methyl); C₁₋₄-alkoxy (especially methoxy); C₁₋₃-fluoroalkyl; halogen; C₃₋₆-cycloalkyl (especially cyclopropyl); C₃₋₆-cycloalkyl-methyl; C₁₋₃-alkoxy-C₁₋₃-alkyl; C₂₋₃-alkynyl, benzyl; or phenyl which is unsubstituted, mono- or di-substituted wherein the substituents independently are C₁₋₄-alkyl (especially methyl), C₁₋₄-alkoxy (especially methoxy), or C₁₋₄-fluoroalkoxy (especially trifluoromethoxy);
   —CH₂—CH₂—HET², wherein HET² represents 9- or 10-membered bicyclic heteroaryl (especially benzoxazolyl, benzisoxazolyl, benzofuranyl, benzo[d][1,2,3]triazolyl or [1,2,4]triazolo[1,5-a]pyrimidinyl), wherein said HET² is unsubstituted;
   —CH₂—CH₂—HCy¹, wherein HCy¹ represents a partially aromatic bicyclic ring system consisting of a phenyl ring which is fused to a 5- to 7-membered saturated heterocyclic ring containing one or two heteroatoms independently selected from oxygen and nitrogen, wherein, if present, said nitrogen when having a free valency is unsubstituted or mono-substituted with C₁₋₄-alkyl (especially methyl); and wherein the phenyl ring of said partially aromatic bicyclic ring system is unsubstituted, mono-, or di-substituted, wherein the substituents are independently C₁₋₄-alkyl (especially methyl), C₁₋₄-alkoxy (especially methoxy), or halogen (especially chloro, bromo); or
   —CH₂—CH₂—HCy², wherein HCy² represents a partially aromatic bicyclic ring system consisting of a 5-membered heteroaryl which is fused to a 5- to 7-membered saturated carbocyclic ring.
10) Another embodiment relates to compounds according to any one of embodiments 1) to 8), wherein R⁴ represents a group —CO—NH—R⁴¹; wherein R⁴¹ represents
   -L¹-aryl; wherein L¹ represents —CH₂—CH₂—, or —CH₂—CH₂—O—*; wherein asterisks indicate the bond with which L¹ is attached to the aryl; wherein aryl represents phenyl; wherein said aryl independently is unsubstituted, mono-, di- or tri-substituted, wherein the substituents are independently C₁₋₄-alkyl (especially methyl, ethyl, tert-butyl), C₁₋₄-alkoxy (especially methoxy, ethoxy), C₁₋₃-fluoroalkyl, C₁₋₃-fluoroalkoxy, halogen (especially fluoro, chloro, bromo), hydroxy-C₁₋₃-alkyl, 5- or 6-membered heteroaryl (especially pyrazolyl, triazolyl, pyrimidinyl, pyrazinyl), or —NR^{N41}R^{N42}, wherein independently R^{N41} is hydrogen or C₁₋₄-alkyl, and R^{N42} is hydrogen or C₁₋₄-alkyl;
   -L²-HET¹; wherein L² represents —CH₂—CH₂—, —CH₂—CF₂—*, or —CH₂—CH(OH)—*; wherein asterisks indicate the bond with which L² is attached to HET¹ (especially L² represents —CH₂—CH₂—); wherein HET¹ represents 5- or 6-membered heteroaryl (especially thiophenyl, furanyl, thiazolyl, isothiazolyl, pyrazolyl, isoxazolyl, oxadiazolyl, triazolyl, tetrazolyl, pyridinyl, pyrimidinyl, pyrazinyl), wherein said 5- or 6-membered heteroaryl is independently unsubstituted, mono-, or di-substituted, wherein the substituents are independently C₁₋₄-alkyl (especially methyl, tert-butyl); C₁₋₄-alkoxy (especially methoxy); C₁₋₃-fluoroalkyl; halogen; C₃₋₆-cycloalkyl; benzyl; or phenyl which is unsubstituted, or mono-substituted wherein the substituents independently are C₁₋₄-alkyl (especially methyl), C₁₋₄-alkoxy (especially methoxy), or C₁₋₄-fluoroalkoxy (especially trifluoromethoxy);
   —CH₂—CH₂—HET², wherein HET² represents 9- or 10-membered bicyclic heteroaryl (especially benzoxazolyl, benzisoxazolyl, benzofuranyl, benzo[d][1,2,3]triazolyl or [1,2,4]triazolo[1,5-a]pyrimidinyl), wherein said HET² is unsubstituted;
   CH₂—CH₂—HCy¹, wherein HCy¹ represents a partially aromatic bicyclic ring system consisting of a phenyl ring which is fused to a 5- to 7-membered saturated heterocyclic ring containing one or two oxygen atoms; wherein the phenyl ring of said partially aromatic bicyclic ring system is unsubstituted, or mono-substituted, wherein the substituents are independently C₁₋₄-alkyl (especially methyl), C₁₋₄-alkoxy (especially methoxy), or halogen (especially chloro, bromo); or
   —CH₂—CH₂—HCy², wherein HCy² represents a partially aromatic bicyclic ring system consisting of a 5-membered heteroaryl which is fused to a 5- to 7-membered saturated carbocyclic ring.
11) Another embodiment relates to compounds according to any one of embodiments 1) to 8), wherein R⁴ represents a group —CO—NH—R⁴¹; wherein R⁴¹ represents a group selected from:
A)

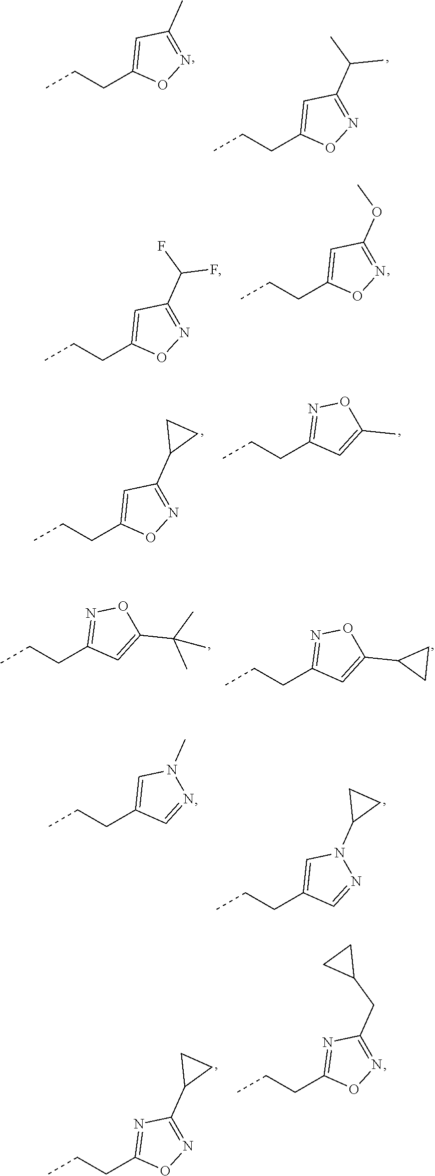

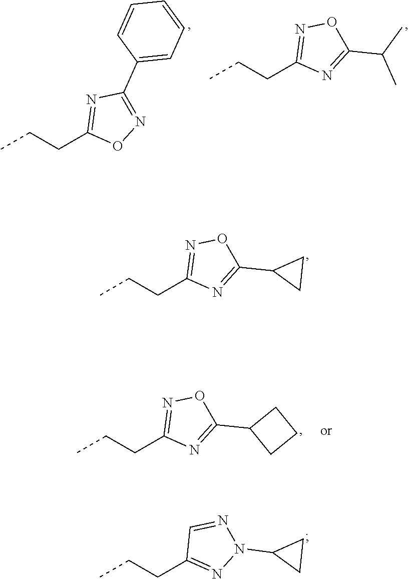

B)

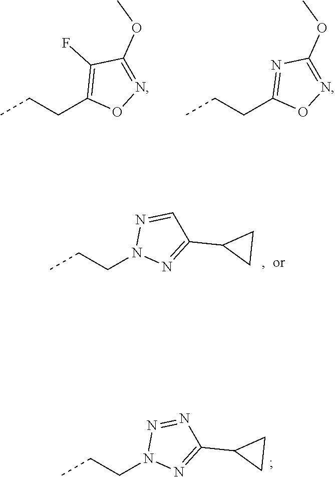

C)

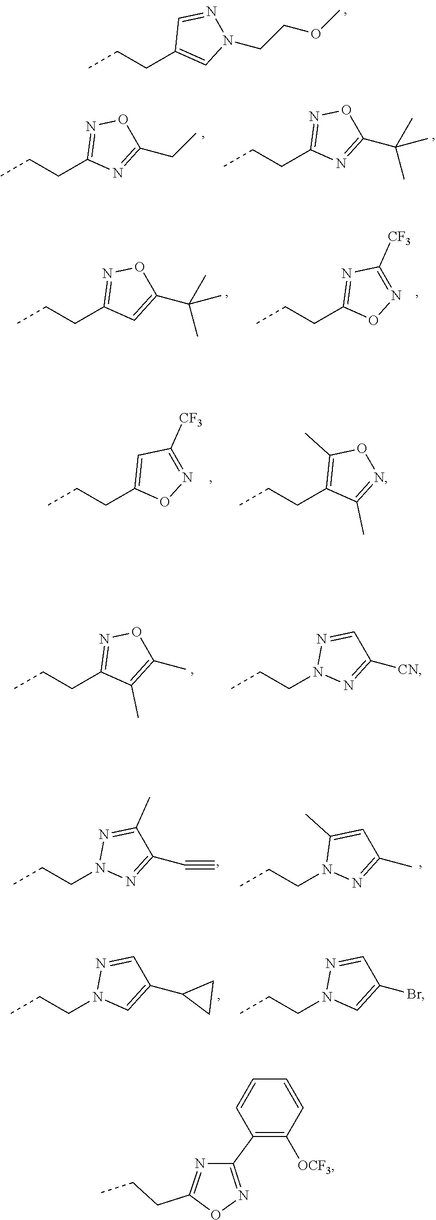

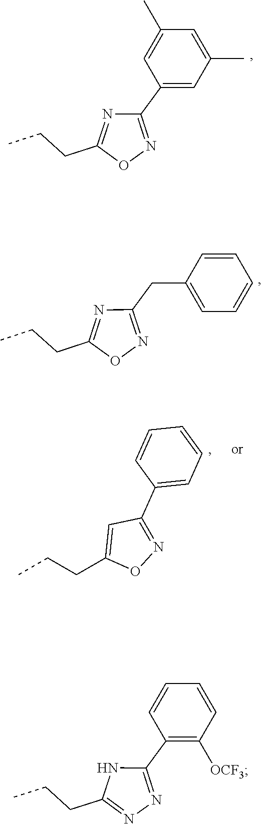

D)

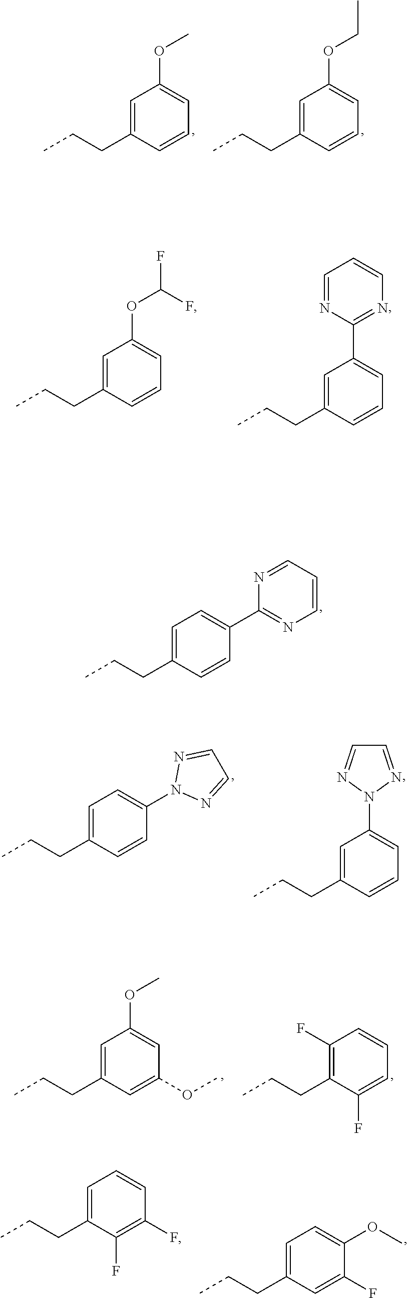

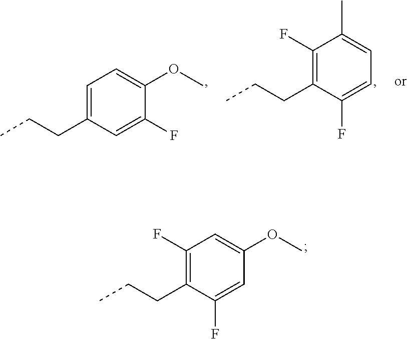

E)

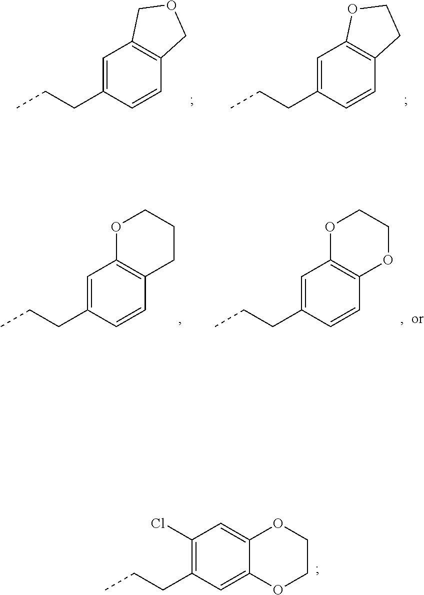

F)

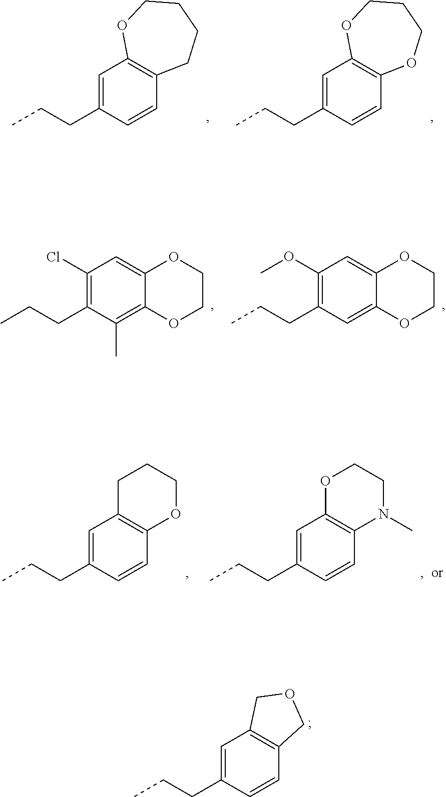

G)

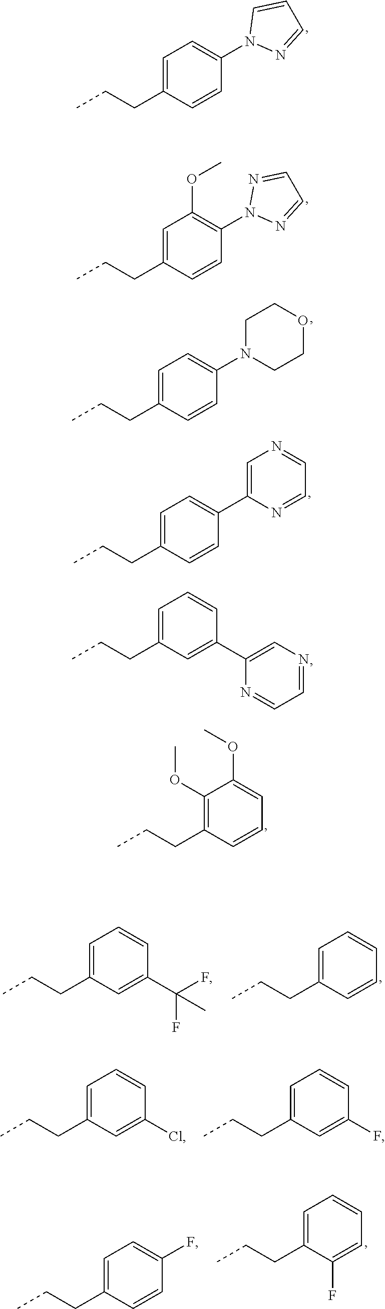

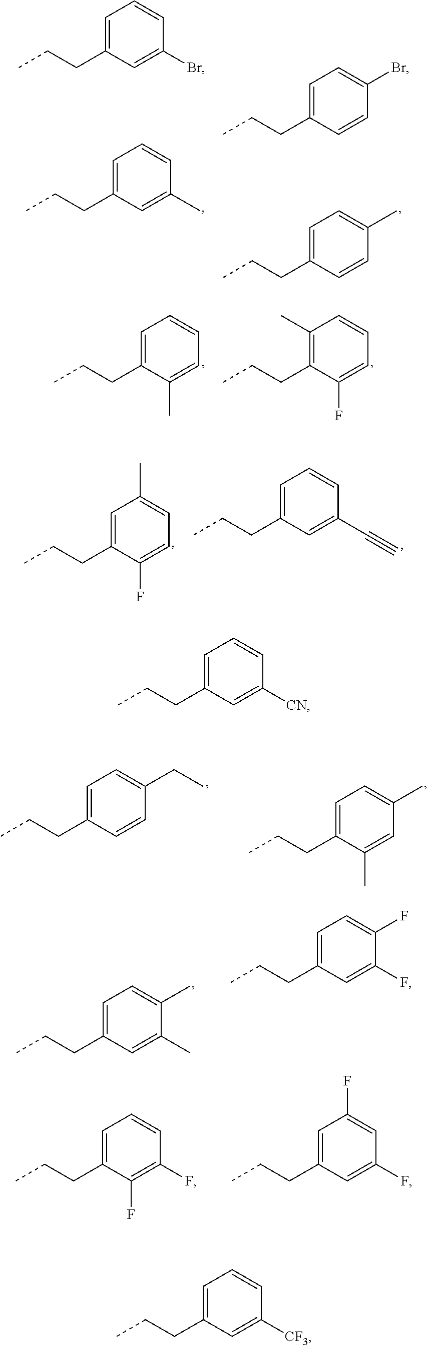

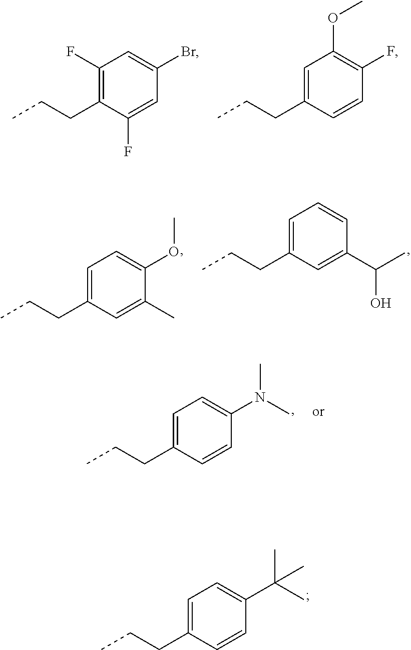

H)

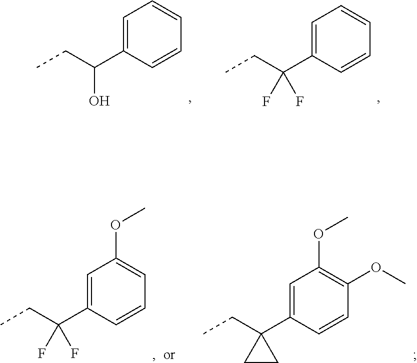

I)

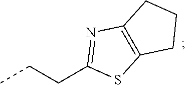

J)

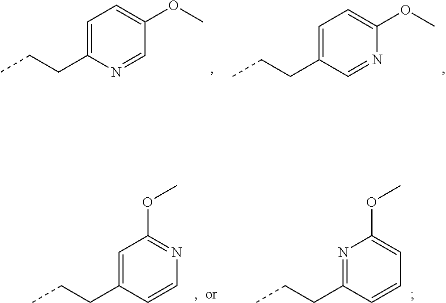

K)

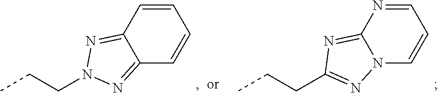

L)

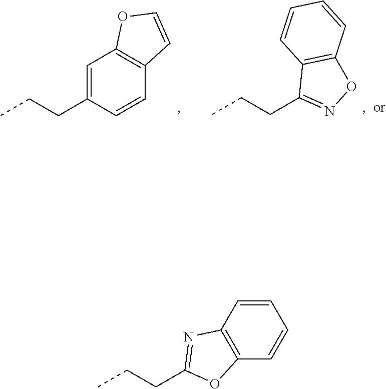

M)

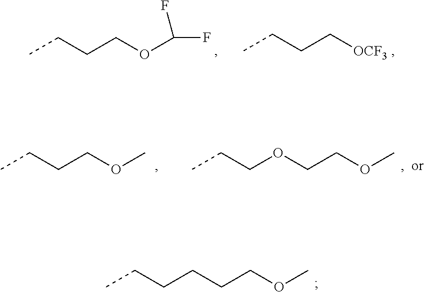

N)

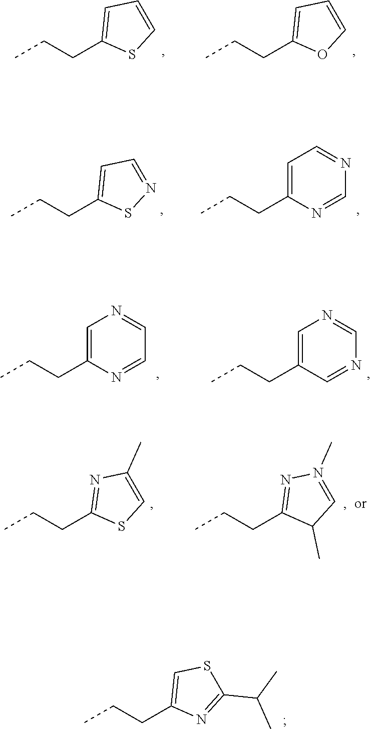

O)

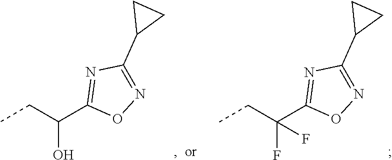

P)

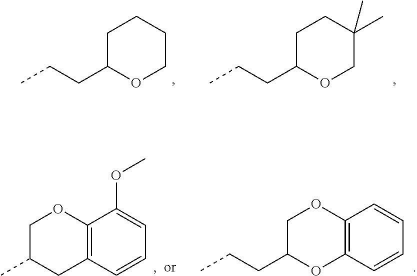

wherein the above groups A), B), C), D), E), F), G), H), I), J), K), L), M), N), O), and P) each form a particular sub-embodiment; wherein notably the groups A) and B) together form a particular sub-embodiment, and the groups D) and E) together form another particular sub-embodiment. Another particular sub-embodiment is formed by the groups A), D), E), and I), especially A), D), and E). Another particular sub-embodiment is formed by the groups A), B), D), E), I), and K), especially A), B), D) and K).
12) Another embodiment relates to compounds according to any one of embodiments 1) to 11), wherein Ar² represents phenyl.
13) Another embodiment relates to compounds according to any one of embodiments 1) to 12), wherein Ar¹ represents
   phenylene, or 5- or 6-membered heteroarylene; wherein said phenylene, or 5- or 6-membered heteroarylene independently is mono-, or di-substituted, wherein the substituents are independently selected from C₁₋₄-alkyl (especially methyl), C₁₋₄-alkoxy (especially methoxy, ethoxy), and halogen (especially fluoro, chloro);
   phenylene wherein said phenylene is fused to a 5- or 6-membered saturated heterocyclic ring containing one or two oxygen atoms, wherein said 5- or 6-membered saturated heterocyclic ring independently is unsubstituted; or
   a bicyclic aromatic ring selected from naphthylene and 8- to 10-membered bicyclic heteroarylene; wherein said bicyclic aromatic ring independently is unsubstituted, or mono-substituted, wherein the substituents are independently selected from C₁₋₄-alkyl (especially methyl), and halogen (especially fluoro, chloro);
   [wherein it is understood that in the above groups Ar¹ the —CO— group and the oxygen (i.e. the groups linking Ar¹ to the rest of the molecule) are attached in ortho arrangement to aromatic ring carbon atoms of Ar¹].
14) Another embodiment relates to compounds according to any one of embodiments 1) to 12), wherein Ar¹ represents a group selected from
A)

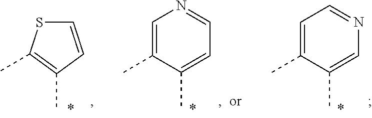

B)

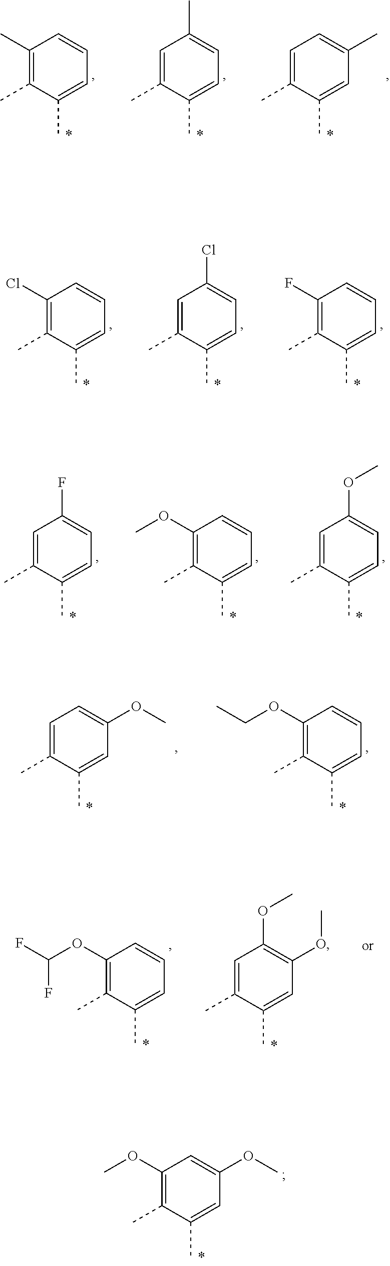

or, in addition,

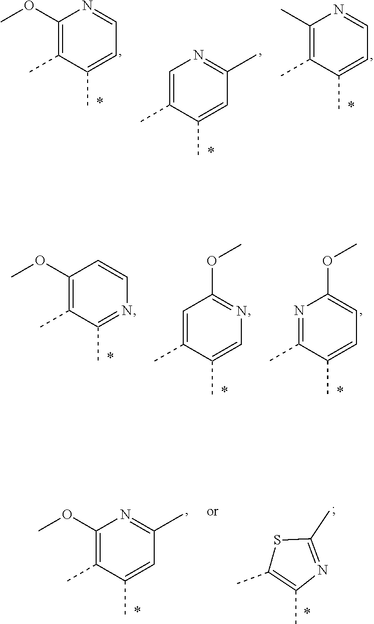

C)
or, in addition,

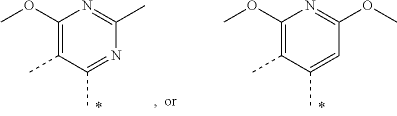

D)

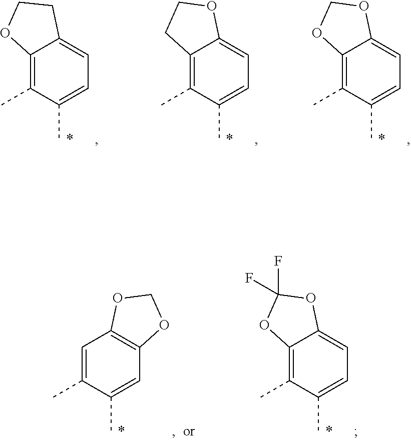

E)

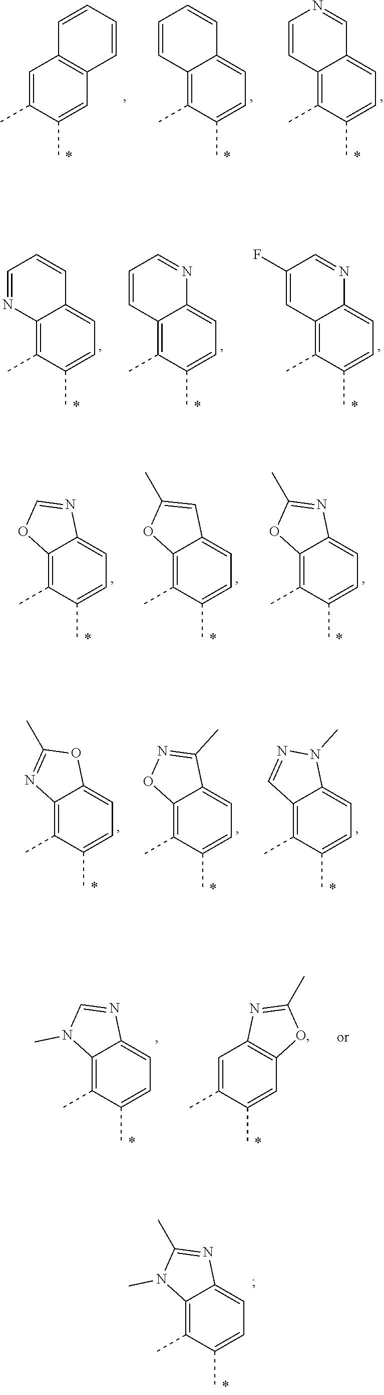

or, in addition,

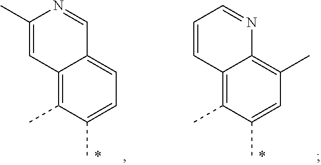

or
F)

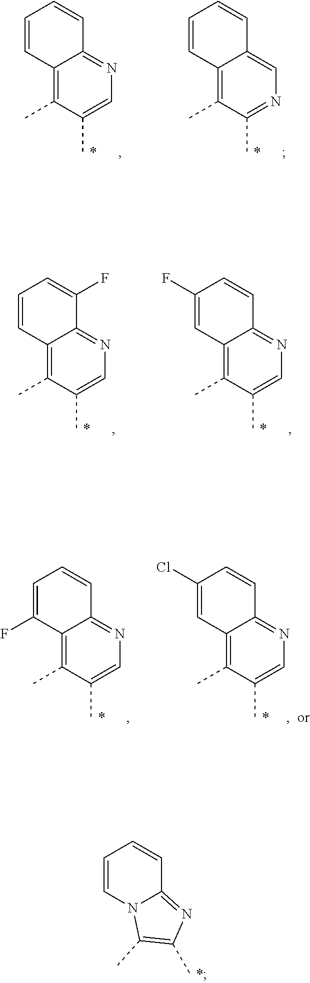

or, in addition,

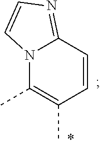

or
G)

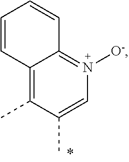

or, in addition,

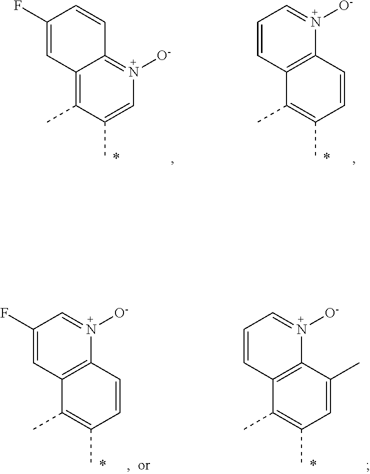

wherein in the above groups the asterisks indicate the bond with which said groups are attached to the oxygen (i.e. to the oxygen linking Ar¹ to the rest of the molecule);
wherein the above groups A), B), C), D), E), F) and G) each form a particular sub-embodiment.
15) A second aspect of the invention relates to compounds of the Formula (II)

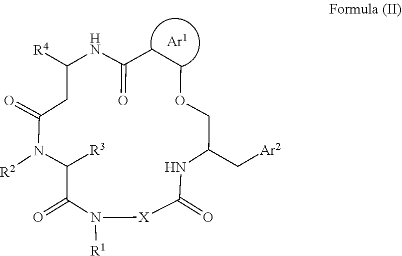

for use in the treatment of cystic fibrosis;
wherein X, R¹, R², R³, R⁴, Ar² independently are as defined for the compounds of Formula (I) in any one of embodiments 1), 2) or 4) to 12); and
Ar¹ represents
   phenylene wherein said phenylene is unsubstituted;
   5- or 6-membered heteroarylene wherein said 5- or 6-membered heteroarylene is unsubstituted (especially pyridin-3,4-diyl, thiophen-2,3-diyl);
   phenylene, or 5- or 6-membered heteroarylene; wherein said phenylene, or 5- or 6-membered heteroarylene independently is mono-, di- or tri-substituted, wherein the substituents are independently selected from C₁₋₄-alkyl (especially methyl), C₁₋₄-alkoxy (especially methoxy, ethoxy), C₁₋₃-fluoroalkyl, C₁₋₃-fluoroalkoxy, cyano, and halogen (especially fluoro, chloro);
   phenylene wherein said phenylene is fused to a 5- or 6-membered saturated heterocyclic ring containing one or two oxygen atoms, wherein said 5- or 6-membered saturated heterocyclic ring independently is unsubstituted or di-substituted with fluoro; or
   a bicyclic ring selected from naphthylene and 8- to 10-membered bicyclic heteroarylene; wherein said bicyclic ring independently is unsubstituted, mono-, or di-substituted, wherein the substituents are independently selected from C₁₋₄-alkyl (especially methyl), C₁₋₃-fluoroalkyl, C₁₋₄-alkoxy, C₁₋₃-fluoroalkoxy, cyano, and halogen (especially fluoro, chloro); or
   quinoline-diyl, wherein said quinoline-diyl is present in form of the respective N-oxide; wherein said quinoline-diyl N-oxide is unsubstituted or mono-substituted with methyl or fluoro;
[wherein it is understood that in the above groups Ar¹ the —CO— group and the oxygen (i.e. the groups linking Ar¹ to the rest of the molecule) are attached in ortho arrangement to aromatic ring carbon atoms of Ar¹].
16) Another embodiment relates to compounds of the Formula (II) according to embodiment 15), for use in the treatment of cystic fibrosis; wherein Ar¹ represents unsubstituted phenylene; or represents a group as defined in embodiment 13) or 14).
17) A further embodiment relates to compounds of Formula (II) for use according to embodiment 15) or 16), wherein the compounds are compounds of Formula (II_{E}):

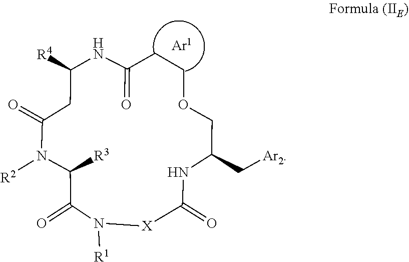

The compounds of formula (I)/formula (II) contain at least three stereogenic or asymmetric centers, which are present in (R)- or (S)-configuration as defined in the respective embodiment defining such compound of formula (I)/formula (II). In addition, the compounds of formula (I)/formula (II) may contain one or more further stereogenic or asymmetric centers, such as one or more additional asymmetric carbon atoms. The compounds of formula (I)/formula (II) may thus be present as mixtures of stereoisomers or preferably as pure stereoisomers. Mixtures of stereoisomers may be separated in a manner known to a person skilled in the art. In case any stereogenic or asymmetric center in a given chemical name is designated as being in (RS)-configuration, this means that such stereogenic or asymmetric center in such compound may be present in (R)-configuration, in (S)-configuration, or in any mixture of epimers with regard to such center.

Thus, for example the compound (3S,7S,10RS,13R)-13-benzyl-10-(tert-butoxymethyl)-7-isobutyl-N-(3-methoxyphenethyl)-6,9-dimethyl-1,5,8,11-tetraoxo-1,2,3,4,5,6,7,8,9,10,11,12,13,14-tetradecahydronaphtho[1,2-p][1]oxa[4,7,10,14]tetraazacycloheptadecine-3-carboxamide comprises (3S,7S,10R,13R)-13-benzyl-10-(tert-butoxymethyl)-7-isobutyl-N-(3-methoxyphenethyl)-6,9-dimethyl-1,5,8,11-tetraoxo-1,2,3,4,5,6,7,8,9,10,11,12,13,14-tetradecahydronaphtho[1,2-p][1]oxa[4,7,10,14]tetraazacycloheptadecine-3-carboxamide, the compound (3S,7S,10S,13R)-13-benzyl-10-(tert-butoxymethyl)-7-isobutyl-N-(3-methoxyphenethyl)-6,9-dimethyl-1,5,8,11-tetraoxo-1,2,3,4,5,6,7,8,9,10,11,12,13,14-tetradecahydronaphtho[1,2-p][1]oxa[4,7,10,14]tetraazacycloheptadecine-3-carboxamide, and any mixture thereof. Likewise, in a certain chemical structure (such as in Table 3, 4, and 5), a stereogenic or asymmetric center indicated as “abs” represents said stereogenic or asymmetric center in the respective (R)- or (S)-configuration. A stereogenic or asymmetric center indicated as “&1” represents said stereogenic or asymmetric center in the respective (RS)-configuration, i.e. comprising the respective (R)- or (S)-configuration or any mixture of epimers at such center.

The compounds of formula (I)/formula (II) may further encompass compounds with one or more double bonds which are allowed to be present in Z- as well as E-configuration and/or compounds with substituents at a ring system which are allowed to be present, relative to each other, in cis- as well as trans-configuration.

In case a particular compound (or generic structure) is designated as (R)- or (S)-enantiomer, such designation is to be understood as referring to the respective compound (or generic structure) in enriched enantiomeric form, especially in essentially pure enantiomeric form. Likewise, in case a specific asymmetric center in a compound is designated as being in (R)- or (S)-configuration or as being in a certain relative configuration, such designation is to be understood as referring to the compound that is in enriched, especially essentially pure form with regard to the respective configuration of said asymmetric center. In analogy, cis- or trans-designations are to be understood as referring to the respective stereoisomer of the respective relative configuration in enriched, especially essentially pure form. Likewise, in case a particular compound (or generic structure) is designated as Z- or E-stereoisomer (or in case a specific double bond in a compound is designated as being in Z- or E-configuration), such designation is to be understood as referring to the respective compound (or generic structure) in enriched, especially essentially pure stereoisomeric form (or to the compound that is in enriched, especially essentially pure, form with regard to the respective configuration of the double bond).

The term “enriched”, when used in the context of stereoisomers, is to be understood in the context of the present invention to mean that the respective stereoisomer is present in a ratio of at least 70:30, especially of at least 90:10 (i.e., in a purity of at least 70% by weight, especially of at least 90% by weight), with regard to the respective other stereoisomer/the entirety of the respective other stereoisomers.

The term “essentially pure”, when used in the context of stereoisomers, is to be understood in the context of the present invention to mean that the respective stereoisomer is present in a purity of at least 95% by weight, especially of at least 99% by weight, with regard to the respective other stereoisomer/the entirety of the respective other stereoisomers.

The present invention also includes isotopically labelled, especially ²H (deuterium) labelled compounds of formula (I)/formula (II) according to embodiments 1) to 21), which compounds are identical to the compounds of formula (I)/formula (II) except that one or more atoms have each been replaced by an atom having the same atomic number but an atomic mass different from the atomic mass usually found in nature. Isotopically labelled, especially ²H (deuterium) labelled compounds of formula (I)/formula (II) and salts thereof are within the scope of the present invention. In case a certain substituent is specifically indicated as representing hydrogen, it is understood to refer to all isotopes of the atom “H”, i.e. the term hydrogen as used for a certain substituent is understood as comprising the isotope ²H (deuterium); preferably it refers to the isotope ¹H (hydrogen). Substitution of hydrogen with the heavier isotope ²H (deuterium) may lead to greater metabolic stability, resulting e.g. in increased in-vivo half-life or reduced dosage requirements, or may lead to reduced inhibition of cytochrome P450 enzymes, resulting e.g. in an improved safety profile. In one embodiment of the invention, the compounds of formula (I)/formula (II) are not isotopically labelled, or they are labelled only with one or more deuterium atoms. In a sub-embodiment, the compounds of formula (I)/formula (II) are not isotopically labelled at all. Isotopically labelled compounds of formula (I)/formula (II) may be prepared in analogy to the methods described hereinafter, but using the appropriate isotopic variation of suitable reagents or starting materials.

In this patent application, a bond drawn as a dotted line shows the point of attachment of the radical drawn. For example, the radical drawn below

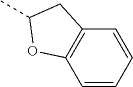

is the 2,3-dihydrobenzofuran-2-yl group.

Where the plural form is used for compounds, salts, pharmaceutical compositions, diseases and the like, this is intended to mean also a single compound, salt, or the like.

Any reference to compounds of formula (I)/formula (II) according to embodiments 1) to 21) is to be understood as referring to the compound in free base or salt form, thus, referring also to the salts (and especially the pharmaceutically acceptable salts) of such compounds, as appropriate and expedient.

The term “pharmaceutically acceptable salts” refers to salts that retain the desired biological activity of the subject compound and exhibit minimal undesired toxicological effects. Such salts include inorganic or organic acid and/or base addition salts depending on the presence of basic and/or acidic groups in the subject compound. For reference see for example “Handbook of Pharmaceutical Salts. Properties, Selection and Use.”, P. Heinrich Stahl, Camille G. Wermuth (Eds.), Wiley-VCH, 2008; and “Pharmaceutical Salts and Co-crystals”, Johan Wouters and Luc Quéré (Eds.), RSC Publishing, 2012.

Definitions provided herein are intended to apply uniformly to the compounds of formula (I)/formula (II), as defined in any one of embodiments 1) to 17), and, mutatis mutandis, throughout the description and the claims unless an otherwise expressly set out definition provides a broader or narrower definition. It is well understood that a definition or preferred definition of a term defines and may replace the respective term independently of (and in combination with) any definition or preferred definition of any or all other terms as defined herein.

Whenever a substituent is denoted as optional, it is understood that such substituent may be absent (i.e. the respective residue is unsubstituted with regard to such optional substituent), in which case all positions having a free valency (to which such optional substituent could have been attached to; such as for example in an aromatic ring the ring carbon atoms and/or the ring nitrogen atoms having a free valency) are substituted with hydrogen where appropriate. Likewise, in case the term “optionally” is used in the context of (ring) heteroatom(s), the term means that either the respective optional heteroatom(s), or the like, are absent (i.e. a certain moiety does not contain heteroatom(s)/is a carbocycle/or the like), or the respective optional heteroatom(s), or the like, are present as explicitly defined.

The term “halogen” means fluorine/fluoro, chlorine/chloro, or bromine/bromo; preferably fluorine/fluoro or chlorine/chloro.

The term “alkyl”, used alone or in combination, refers to a saturated straight or branched chain hydrocarbon group containing one to six carbon atoms. The term “C_{x-y}-alkyl” (x and y each being an integer), refers to an alkyl group as defined before, containing x to y carbon atoms. For example, a C₁₋₆-alkyl group contains from one to six carbon atoms.

Representative examples of alkyl groups are methyl, ethyl, propyl, isopropyl, butyl, isobutyl, tert.-butyl, 3-methyl-butyl, 2,2-dimethyl-propyl and 3,3-dimethyl-butyl. For avoidance of any doubt, in case a group is referred to as e.g. propyl or butyl, it is meant to be n-propyl, respectively n-butyl. In case R^{X2} represents a C₁₋₆-alkyl group, the term especially refers to C₁₋₄-alkyl, in particular to methyl, ethyl, isopropyl, or isobutyl; preferably methyl. In case R¹ represents —C₁₋₃-alkyl, the term especially means methyl, or 3,3-dimethylbut-1-yl; preferably methyl. In case R^{X} represents C₁₋₄-alkyl, the term especially means methyl, ethyl, isopropyl, or isobutyl. For R^{OX1} representing C₁₋₄-alkyl, the term especially means methyl or isobutyl. For R^{OX2} representing C₁₋₄-alkyl, the term especially means methyl or ethyl. For R² representing C₁₋₄-alkyl the term especially means methyl, or ethyl; preferably methyl. In case R³ represents —C₁₋₆-alkyl, the term especially means methyl, or isobutyl; preferably isobutyl. A C₁₋₆-alkyl group wherein said C₁₋₆-alkyl is mono-substituted with R¹¹ especially refers to —(CH₂)ₘ— groups wherein m represents the integer 1 or 2, or to a C₃₋₆-alkyl group, said groups being mono-substituted with R¹¹ as explicitly defined.

The term “—C_{x-y}-alkylene-”, used alone or in combination, refers to bivalently bound alkyl group as defined before containing x to y carbon atoms. Preferably, the points of attachment of a —C₁-y-alkylene group are in 1,1-diyl, in 1,2-diyl, or in 1,3-diyl arrangement.

It is understood that an alkylene group (or a substituted alkyl group) that links two heteroatoms preferably will distance such heteroatoms by at least 2 carbon atoms.

The term “alkoxy”, used alone or in combination, refers to an alkyl-O— group wherein the alkyl group is as defined before. The term “C_{x-y}-alkoxy” (x and y each being an integer) refers to an alkoxy group as defined before containing x to y carbon atoms. For example, a C₁₋₄-alkoxy group means a group of the formula C₁₋₄-alkyl-O— in which the term “C₁₋₄-alkyl” has the previously given significance. Representative examples of alkoxy groups are methoxy, ethoxy, n-propoxy, isopropoxy, n-butoxy, isobutoxy, sec-butoxy and tert-butoxy. Preferred is methoxy.

The term “fluoroalkyl”, used alone or in combination, refers to an alkyl group as defined before containing one to three carbon atoms in which one or more (and possibly all) hydrogen atoms have been replaced with fluorine. The term “C_{x-y}-fluoroalkyl” (x and y each being an integer) refers to a fluoroalkyl group as defined before containing x to y carbon atoms. For example, a C₁₋₃-fluoroalkyl group contains from one to three carbon atoms in which one to seven hydrogen atoms have been replaced with fluorine. Representative examples of fluoroalkyl groups include especially C₁-fluoroalkyl groups such as trifluoromethyl, and difluoromethyl, as well as 2-fluoroethyl, 2,2-difluoroethyl and 2,2,2-trifluoroethyl. In case R^{X2} represents C₁₋₄-fluoroalkyl, the term especially means 2,2-difluoroethyl or 2,2,2-trifluoroethyl.

The term “—C_{x-y}-fluoroalkylene-”, used alone or in combination, refers to bivalently bound fluoroalkyl group as defined before containing x to y carbon atoms.

The term “fluoroalkoxy”, used alone or in combination, refers to an alkoxy group as defined before containing one to three carbon atoms in which one or more (and possibly all) hydrogen atoms have been replaced with fluorine. The term “C_{x-y}-fluoroalkoxy” (x and y each being an integer) refers to a fluoroalkoxy group as defined before containing x to y carbon atoms. For example, a C₁₋₃-fluoroalkoxy group contains from one to three carbon atoms in which one to seven hydrogen atoms have been replaced with fluorine. Representative examples of fluoroalkoxy groups include trifluoromethoxy, difluoromethoxy, 2-fluoroethoxy, 2,2-difluoroethoxy and 2,2,2-trifluoroethoxy. Preferred are (C₁)fluoroalkoxy groups such as trifluoromethoxy and difluoromethoxy.

The term “alkynyl”, used alone or in combination, refers to a straight or branched hydrocarbon chain containing two to four carbon atoms and one carbon-carbon triple bond. The term “C_{x-y}-alkynyl” (x and y each being an integer), refers to an alkynyl group as defined before containing x to y carbon atoms. For example, a C₂₋₄-alkynyl group contains from two to four carbon atoms. In case R³ represents —C₂₋₄-alkynyl, the term especially means prop-1-yn-3-yl. An example of C₂₋₃-alkynyl is ethynyl.

The term “cycloalkyl”, used alone or in combination, refers to a saturated monocyclic hydrocarbon ring containing three to six carbon atoms. The term “C_{x-y}cycloalkyl” (x and y each being an integer), refers to a cycloalkyl group as defined before containing x to y carbon atoms. For example, a C₃₋₆-cycloalkyl group contains from three to six carbon atoms. Examples of cycloalkyl groups are cyclopropyl, cyclobutyl, cyclopentyl, and cyclohexyl. In case R^{X2} represents C₃₋₆-cycloalkyl, the term preferably means cyclopropyl, cyclobutyl, or cyclopentyl. In case R¹¹ represents C₃₋₆-cycloalkyl, the term especially means cyclobutyl or cyclohexyl.

The term “—C_{x-y}-cycloalkylene-”, used alone or in combination, refers to bivalently bound cycloalkyl group as defined before containing x to y carbon atoms. Preferably, the points of attachment of any bivalently bound cycloalkyl group are in 1,1-diyl arrangement. Examples are cyclopropan-1,1-diyl, cyclobutan-1,1-diyl, and cyclopentan-1,1-diyl; preferred is cyclopropan-1,1-diyl.

Examples of C₃₋₆-cycloalkan-1,1-diyl- are cyclopropan-1,1-diyl, cyclobutan-1,1-diyl and cyclopentane-1,1-diyl. An example of a C₅₋₆-cycloalkan-1,1-diyl- group which is fused to a benzene ring is 1,3-dihydro-2H-indene-2,2-diyl.

The term “heterocycloalkyl”, used alone or in combination, and if not explicitly defined in a broader or more narrow way, refers to a saturated monocyclic hydrocarbon ring containing one or two ring heteroatoms independently selected from nitrogen, sulfur, and oxygen. The term “C_{x-y}-heterocycloalkyl” refers to such a heterocycle containing x to y ring atoms. Examples are tetrahydrofuranyl, terahydropyranyl, and piperidnyl. Heterocycloalkyl groups are unsubstituted or substituted as explicitly defined. In case R¹¹ represents a saturated 5- or 6-membered heterocycloalkyl containing one or two ring heteroatoms, the term especially means tetrahydropyranyl and tetrahydrofuranyl. An example of a C₅₋₆-heterocycloalkyl group containing one ring oxygen atom is especially tetrahydropyranyl.

The term “C₄₋₆-heterocycloalkan-diyl wherein said C₄₋₆-heterocycloalkan-diyl contains one ring oxygen atom” refers to a bivalently bound heterocycloalkyl group containing one ring oxygen atom and the remaining ring carbon atoms. An example of “C₄₋₆-heterocycloalkan-diyl wherein said C₄₋₆-heterocycloalkan-diyl contains one ring oxygen atom” is tetrahydropyran-4,4-diyl. The term “C₄₋₆-heterocycloalkan-diyl wherein said C₄₋₆-heterocycloalkan-diyl contains one ring nitrogen atom”, refers to a bivalently bound heterocycloalkyl group containing one ring nitrogen atom and the remaining ring carbon atoms. An example of “C₄₋₆-heterocycloalkan-diyl wherein said C₄₋₆-heterocycloalkan-diyl contains one ring nitrogen atom” is piperidin-4,4-diyl.

The term “aryl”, used alone or in combination, means phenyl or naphthyl, especially phenyl. The above-mentioned aryl groups are unsubstituted or substituted as explicitly defined.

It is understood that a heterocyclic ring, for example “containing one or two heteroatoms independently selected from oxygen and nitrogen” or “containing one oxygen atom”, contains exactly the number and type of heteroatoms indicated, the remaining ring atoms being carbon atoms if not explicitly indicated otherwise.

Examples of the substituent “HCy¹ representing a partially aromatic bicyclic ring system consisting of a phenyl ring which is fused to a 5- to 7-membered saturated heterocyclic ring containing one or two heteroatoms independently selected from oxygen and nitrogen” are benzodioxolyl, dihydrobenzofuranyl, dihydrobenzodioxinyl, chromanyl, tetrahydrobenzooxepinyl, dihydrobenzooxazinyl; more particularly benzo[d][1,3]dioxol-5-yl, 1,3-dihydroisobenzofuran-5-yl, 2,3-dihydrobenzofuran-5-yl, 2,3-dihydrobenzofuran-6-yl, 2,3-dihydrobenzo[b][1,4]dioxin-6-yl, 2,3-dihydrobenzo[b][1,4]dioxin-2-yl, chroman-6-yl, chroman-7-yl, 2,3,4,5-tetrahydrobenzo[b]oxepin-8-yl, 3,4-dihydro-2H-benzo[b][1,4]dioxepin-7-yl, and 3,4-dihydro-2H-benzo[b][1,4]oxazin-7-yl. The above-mentioned HCy¹ groups are unsubstituted or substituted as explicitly defined.

Preferred example of the substituent “HCy² representing a partially aromatic bicyclic ring system consisting of a 5-membered heteroaryl which is fused to a 5- to 7-membered saturated carbocyclic ring” is 5,6-dihydro-4H-cyclopenta[d]thiazol-2-yl.

A preferred example of the substituent “HCy³ representing a partially aromatic bicyclic ring system consisting of a phenyl ring which is fused to a 5- to 7-membered saturated heterocyclic ring containing one oxygen atom” is chroman-3-yl.

Examples of the group Ar¹/the fragment:

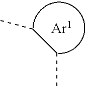

are:
phenylene or 5- or 6-membered heteroarylene such as thiophene-diyl, thiazole-diyl, or pyridine-diyl;
phenylene wherein said phenylene is is fused to a 5- or 6-membered saturated heterocyclic ring containing one or two oxygen atoms, such as benzo[d][1,3]dioxole-diyl, or 2,3-dihydrobenzofuran-diyl;
a bicyclic aromatic ring selected from naphthylene and 8- to 10-membered bicyclic heteroarylene, such as naphthalene-diyl, benzofuran-diyl, benzo[d]oxazole-diyl, benzo[d]isoxazole-diyl, imidazo[1,2-a]pyridine-diyl, 1H-indazole-diyl, 1H-benzo[d]imidazole-diyl, quinoline-diyl, or isoquinoline-diyl; and
quinoline-diyl, wherein such quinoline-diyl is present in form of the respective N-oxide, such as quinoline-1-oxide-diyl.

Examples of the group Ar¹ are especially those, notably as listed above, with the —CO— group and the oxygen (i.e. the groups linking Ar¹ to the rest of the molecule) attached in ortho arrangement to aromatic ring carbon atoms of Ar. In addition, said groups Ar¹ are unsubstituted or substituted as explicitly defined.

Particular examples of the fragment:

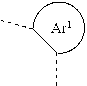

are:
phenylene or 5- or 6-membered heteroarylene, such as 1,2-phenylene, thiophene-2,3-diyl, thiazole-4,5-diyl, pyridine-3,4-diyl, or pyridine-2,3-diyl;
phenylene wherein said phenylene is is fused to a 5- or 6-membered saturated heterocyclic ring containing one or two oxygen atoms, such as benzo[d][1,3]dioxole-4,5-diyl, benzo[d][1,3]dioxole-5,6-diyl, 2,3-dihydrobenzofuran-6,7-diyl, or 2,3-dihydrobenzofuran-4,5-diyl;
a bicyclic aromatic ring selected from naphthylene and 8- to 10-membered bicyclic heteroarylene, such as naphthalene-1,2-diyl, naphthalene-2,3-diyl, benzofuran-6,7-diyl, benzo[d]oxazole-4,5-diyl, benzo[d]oxazole-5,6-diyl, benzo[d]oxazole-6,7-diyl, benzo[d]isoxazole-6,7-diyl, imidazo[1,2-a]pyridine-2,3-diyl, 1H-indazole-4,5-diyl, 1H-benzo[d]imidazole-6,7-diyl, quinoline-7,8-diyl, quinoline-3,4-diyl, quinoline-5,6-diyl, isoquinoline-3,4-diyl or isoquinoline-5,6-diyl;
quinoline-diyl, wherein such quinoline-diyl is present in form of the respective N-oxide, such as quinoline-1-oxide-3,4-diyl or quinoline-1-oxide-5,6-diyl.

The above-mentioned groups Ar¹ are unsubstituted or substituted as explicitly defined.

The term “heteroaryl”, used alone or in combination, and if not explicitly defined in a broader or more narrow way, means a 5- to 10-membered monocyclic or bicyclic aromatic ring containing one to a maximum of four heteroatoms, each independently selected from oxygen, nitrogen and sulfur. Representative examples of such heteroaryl groups are 5-membered heteroaryl groups such as furanyl, oxazolyl, isoxazolyl, oxadiazolyl, thiophenyl, thiazolyl, isothiazolyl, thiadiazolyl, pyrrolyl, imidazolyl, pyrazolyl, triazolyl, tetrazolyl; 6-membered heteroaryl groups such as pyridinyl, pyrimidinyl, pyridazinyl, pyrazinyl; and 8- to 10-membered bicyclic heteroaryl groups such as indolyl, isoindolyl, benzofuranyl, isobenzofuranyl, benzothiophenyl, indazolyl, benzimidazolyl, benzoxazolyl, benzisoxazolyl, benzothiazolyl, benzoisothiazolyl, benzotriazolyl, benzoxadiazolyl, benzothiadiazolyl, thienopyridinyl, quinolinyl, isoquinolinyl, naphthyridinyl, cinnolinyl, quinazolinyl, quinoxalinyl, phthalazinyl, pyrrolopyridinyl, pyrazolopyridinyl, pyrazolopyrimidinyl, pyrrolopyrazinyl, imidazopyridinyl, imidazopyridazinyl, and imidazothiazolyl. The above-mentioned heteroaryl groups are unsubstituted or substituted as explicitly defined.

In case R^{OX1} represents 5- or 6-membered heteroaryl, the term especially means 6-membered heteroaryl containing one or two nitrogen atoms such as pyrazinyl or pyridinyl.

For the substituent HET¹ representing a “5- or 6-membered heteroaryl”, the term especially means the above-mentioned 5- or 6-membered groups such as especially pyridinyl, pyrimidinyl, pyrazinyl, furanyl, pyrazolyl, triazolyl, tetrazolyl, isoxazolyl, oxadiazolyl, thiazolyl, isothiazolyl or thiophenyl. Notably, the term refers to 5-membered groups such as especially thiophen-2-yl, thiazol-2-yl, thiazol-4-yl, 1,2,3-triazol-4-yl, 1,2,4-triazol-3-yl, furan-2-yl, isothiazol-5-yl, pyrazol-1-yl, pyrazol-3-yl, pyrazol-4-yl, isoxazol-3-yl, isoxazol-4-yl, isoxazol-5-yl, 1,2,4-oxadiazol-5-yl, 1,2,4-oxadiazol-3-yl, 2H-[1,2,3]triazol-2-yl, 2H-[1,2,3]triazol-4-yl, 2H-tetrazol-2-yl; and 6-membered groups such as especially pyridin-2-yl, pyridin-4-yl, pyrazin-2-yl, pyrimidin-4-yl, pyrimidin-5-yl. The above groups are substituted as explicitly defined.

For the substitutent Ar² representing “5- or 6-membered heteroaryl”, the term especially means pyridinyl, in particular pyridine-2-yl.

For the substituent HET² representing a “9- or 10-membered bicyclic heteroaryl” the term especially refers to benzoxazolyl, benzisoxazolyl, and benzofuranyl; as well as benzo[d][1,2,3]triazolyl or [1,2,4]triazolo[1,5-a]pyrimidinyl.

The above groups are unsubstituted or substituted as explicitly defined. Particular examples are benzofuran-6-yl, benzisoxazol-3-yl, benzoxazol-2-yl, and, in addition, 2H-benzo[d][1,2,3]triazol-2-yl and [1,2,4]triazolo[1,5-a]pyrimidin-2-yl.

For the substitutent Ar² representing “9- or 10-membered heteroaryl”, the term especially means benzothiophenyl, in particular benzothiophen-3-yl.

For the substituent HET representing a “5- to 10-membered heteroaryl”, the term especially means 5- or 6-membered heteroaryl groups, or 8- to 10-membered bicyclic heteroaryl groups as defined before; especially pyridinyl, pyrimidinyl, pyrazinyl, furanyl, pyrazolyl, triazolyl, isoxazolyl, oxadiazolyl, thiazolyl, isothiazolyl, thiophenyl, or benzoxazolyl, benzisoxazolyl, benzofuranyl; or, in addition, benzo[d][1,2,3]triazolyl or [1,2,4]triazolo[1,5-a]pyrimidinyl. The above groups are unsubstituted or substituted as explicitly defined.

For the substitutent Ar^{X2} representing 5- to 10-membered heteroaryl such heteroaryl is as defined before; especially it represents mono-cyclic 5- or 6-membered heteroaryl [notably 5-membered heteroaryl containing one to three heteroatoms selected from oxygen and nitrogen (especially oxadiazolyl, triazolyl, or isoxazolyl); or 6-membered heteroaryl containing one or two nitrogen atoms (especially pyridinyl)], wherein such mono-cyclic heteroaryl is unsubstituted or substituted as explicitly defined; or it represents bicyclic 8- to 10-membered heteroaryl [notably 10-membered heteroaryl containing one nitrogen atom (especially quinolinyl)], wherein such bicyclic heteroaryl is notably unsubstituted, or substituted as explicitly defined. Particular examples of the substitutent Ar^{X2} representing 5- to 10-membered heteroaryl are 3-phenyl-[1,2,4]-oxadiazol-5-yl, 3-(5-fluoro-pyridin-2-yl)-[1,2,4]-oxadiazol-5-yl, or 3-trifluoromethyl-[1,2,4]-oxadiazol-5-yl.

For the substitutent Ar^{X3} representing 5- or 6-membered heteroaryl such heteroaryl notably represents 6-membered heteroaryl containing one or two nitrogen atoms, especially pyridinyl; wherein such 5- or 6-membered heteroaryl heteroaryl is unsubstituted or substituted as explicitly defined. A particular example is 5-fluoro-pyridin-2-yl.

Examples of the fragment:

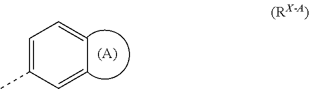

are the 4-methyl-3,4-dihydro-2H-benzo[b][1,4]oxazin-7-yl and 2-oxo-2,3-dihydrobenzo[d]oxazol-5-yl.

The term “cyano” refers to a group —CN.

The term “oxo” refers to a group ═O which is preferably attached to a chain or ring carbon atom as for example in a carbonyl group —(CO)—.

In some instances, the compounds of formula (I)/formula (II) may contain tautomeric forms. Such tautomeric forms are encompassed in the scope of the present invention. In case tautomeric forms exist of a certain residue, and only one form of such residue is disclosed or defined, the other tautomeric form(s) are understood to be encompassed in such disclosed residue. For example, 2-oxo-2,3-dihydrobenzo[d]oxazol-yl group is to be understood as also encompassing its tautomeric form (2-hydroxybenzo[d]oxazol-yl).

Whenever the word “between” is used to describe a numerical range, it is to be understood that the end points of the indicated range are explicitly included in the range. For example: if a temperature range is described to be between 40° C. and 80° C., this means that the end points 40° C. and 80° C. are included in the range; or if a variable is defined as being an integer between 1 and 4, this means that the variable is the integer 1, 2, 3, or 4.

Unless used regarding temperatures, the term “about” placed before a numerical value “X” refers in the current application to an interval extending from X minus 10% of X to X plus 10% of X, and preferably to an interval extending from X minus 5% of X to X plus 5% of X. In the particular case of temperatures, the term “about” placed before a temperature “Y” refers in the current application to an interval extending from the temperature Y minus 10° C. to Y plus 10° C., and preferably to an interval extending from Y minus 5° C. to Y plus 5° C. Besides, the term “room temperature” as used herein refers to a temperature of about 25° C.
18) Another embodiment relates to compounds of Formula (I) according to embodiment 1), wherein said compounds are selected from the compounds of example (as disclosed in the experimental part below):
1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; 105; 106; 107; 108; 109; 110; 111; 112; 113; 114; 115; 116; 117; 118; 119; 120; 121; 122; 123; 124; 125; 126; 127; 128; 129; 130; 131; 132; 133; 134; 135; 136; 137; 138; 139; 140; 141; 142; 143; 144; 145; 146; 147; 148; 149; 150; 151; 152; 153; 154; 155; 156; 157; 158; 159; 160; 161; 162; 163; 164; 165; 166; 167; 168; 169; 170; 171; 172; 173; 174; 175; 176; 177; 178; 179; 180; 181; 182; 183; 184; 185; 186; 187; 188; 189; 190; 191; 192; 193; 194; 195; 196; 197; 198; 199; 200; 201; 202; 203; 204; 205; 206; 207; 208; 209; 210; 211; 212; 213; 214; 215; 216; 217; 218; 219; 220; 221; 222; 223; 224; 225; 226; 227; 228; 229; 230; 231; 232; 233; 234; 235; 236; 237; 238; 239; 240; 241; 242; 243; 244; 245; 246; 247; 248; 249; 250; 251; 252; 253; 254; 255; 256; 257; 258; 259; 260; 261; 262; 263; 264; 265; 266; 267; 268; 269; 270; 271; 272; 273; 274; 275; 276; 277; 278; 279; 280; 281; 282; 283; 284; 285; 286; 287; 288; 289; 290; 291; 292; 293; 294; 295; 296; 297; 298; 299; 300; 301; 302; 303; 304; 305; 306; 307; 308; 309; 310; 311; 312; 313; 314; 315; 316; 317; 318; 319; 320; 321; 322; 323; 324; 325; 326; 327; 328; 329; 330; 331; 332; 333; 334; 335; 336; 337; 338; 339; 340; 341; 342; 343; 344; 345; 346; 347; 348; 349; 350; 351; 352; 353; 354; 355; 356; 357; 358; 359; 360; 361; 362; 363; 364; 365; 366; 367; 368; 369; 370; 371; 372; 373; 374; 375; 376; 377; 378; 379; 380; 381; 382; 383; 384; 385; 386; 387; 388; 389; 390; 391; 392; 393; 394; 395; 396; 397; 398; 399; 400; 401; 402; 403; 404; 405; 406; 407; 408; 409; 410; 411; 412; 413; 414; 415; 416; 417; 418; 419; 420; 421; 422; 423; 424; 425; 426; 427; 428; 429; 430; 431; 432; 433; 434; 435; 436; 437; 438; 439; 440; 441; 442; 443; 444; 445; 446; 447; 448; 449; 450; 451; 452; 453; 454; 455; 456; 457; 458; 459; 460; 461; 462; 463; 464; 465; 466; 467; 468; 469; 470; 471; 472; 473; 474; 475; 476; 477; 478; 479; 480; 481; 482; 483; 484; 485; 486; 487; 488; 489; 490; 491; 492; 493; 494; 495; 496; 497; 498; 499; 500; 501; 502; 503; 504; 505; 506; 507; 508; 509; 510; 511; 512; 513; 514; 515; 516; 517; 518; 519; 520; 521; 522; 523; 524; 525; 526; 527; 528; 529; 530; 531; 532; 533; 534; 535; 536; 537; 538; 539; 540; 541; 542; 543; 544; 545; 546; 547; 548; 549; 550; 551; 552; 553; 554; 555; 556; 557; 558; 559; 560; 561; 562; 563; 564; 565; 566; 567; 568; 569; 570; 571; 572; 573; 574; 575; 576; 577; 578; 579; 580; 581; 582; 583; 584; 585; 586; 587; 588; 589; 590; 591; 592; 593; 594; 595; 596; 597; 598; 599; 600; 601; 602; 603; 604; 605; 606; 607; 608; 609; 610; 611; 612; 613; 614; 615; 616; 617; 618; 619; 620; 621; 622; 623; 624; 625; 626; 627; 628; 629; 630; 631; 632; 633; 634; 635; 636; 637; 638; 639; 640; 641; 642; 643; 644; 645; 646; 647; 648; 649; 650; 651; 652; 653; 654; 655; 656; 657; 658; 659; 660; 661; 662; 663; 664; 665; 666; 667; 668; 669; 670; 671; 672; 673; 674; 675; 676; 677; 678; 679; 680; 681; 682; 683; 684; 685; 686; 689; 690; 691; 692; 693; 694; 695; 696; 697; 698; 699; 700; 701; 702; 703; 704; 705; 706; 707; 708; 709; 710; 711; 712; 713; 714; 715; 716; 717; 718; 719; 720; 721; 722; 723; 724; 725; 726; 727; 728; 729; 730; 731; 732; 733; 734; 735; 736; 737; 738; 739; 740; 741; 742; 743; 744; 745; 746; 747; 748; 749; 750; 751; 752; 753; 754; 755; 756; 757; 758; 759; 760; 761; 762; 763; and 764.
19) In addition to the compounds listed in embodiment 18), further compounds of Formula (I) according to embodiment 1) are selected from the compounds of example (as disclosed in the experimental part below):
765; 766; 767; 768; 769; 770; 771; 772; 773; 774; 775; 776; 777; 778; 779; 780; 781; 782; 783; 784; 785; 786; 787; 788; 789; 790; 791; 792; 793; 794; 795; 796; 797; 798; 799; 800; 801; 802; 803; 804; 805; 806; 807; 808; 809; 810; 811; 812; 813; 814; 815; 816; 817; 818; 819; 820; 821; 822; 823; 824; 825; 826; 827; 828; and 829.
20) In addition to the compounds listed in embodiments 18) and 19), further compounds of Formula (I) according to embodiment 1) are selected from the compounds of example (as disclosed in the experimental part below):
830; 831; 832; 833; 834; 835; 836; 837; 838; 839; 840; 841; 842; 843; 844; 845; 846; 847; 848; 849; 850; 851; 852; 853; 854; 855; 856; 857; 858; 859; 860; 861; 862; 863; 864; 865; 866; 867; 868; 869; 870; 871; 872; 873; 874; 875; 876; 877; 878; 879; 880; 881; 882; 883; 884; 885; 886; 887; 888; 889; 890; 891; 892; 893; 894; 895; 896; 897; 898; 899; 900; 901; 902; 903; 904; 905; 906; 907; 908; 909; 910; 911; 912; 913; 914; 915; 916; 917; 918; 919; 920; 921; 922; 923; 924; 925; 926; 927; 928; 929; 930; 931; 932; 933; 934; 935; 936; 937; 938; 939; 940; 941; 942; 943; 944; 945; 946; 947; 948; 949; 950; 951; 952; 953; 954; 955; 956; 957; 958; 959; 960; 961; 962; 963; 964; 965; 966; 967; 968; 969; 970; 971; 972; 973; 974; 975; 976; 977; 978; 979; 980; 981; 982; and 983.
21) Another embodiment relates to compounds of Formula (I) according to embodiment 1), wherein said compounds are selected from the compounds of example (as disclosed in the experimental part below):
72; 127; 131; 132; 140; 171; 256; 275; 276; 281; 282; 315; 316; 355; 372; 374; 379; 380; 381; 383; 389; 391; 393; 395; 509; 524; 538; 539; 548; 563; 571; 578; 610; 613; 617; 618; 637; 640; 641; 655; 660; 665; 672; 673; 682; 692; 693; 697; 713; 714; 716; 717; 719; 721; 722; 724; 769; 778; 791; 794; 795; 801; 804; 808; 809; 811; 813; 815; 820; 822; 827; 829; 838; 839; 840; 843; 849; 851; 853; 854; 855; 856; 858; 859; 865; 867; 868; 871; 873; 874; 875; 876; 878; 879; 880; 883; 884; 885; 886; 887; 889; 890; 895; 897; 898; 902; 903; 908; 909; 918; 919; 923; 924; 927; 930; 934; 935; 936; 937; 941; 944; 945; 948; 949; 960; 962; 964; 965; 966; 967; 969; 970; 972; 973; 975; 978; 980; and 981.

For avoidance of doubt, the chemical names of said example compounds as listed in embodiments 18), 19) and 21) are disclosed in the experimental part; and the corresponding structures of said example compounds are as shown in Table 3, 4 or 5 below, wherein, in case of doubt the depicted structure shall prevail.

Thus, for example the compound of example 713: (3S,7S,10R,13R)-13-benzyl-10-((benzyloxy)methyl)-7-isobutyl-N-(3-methoxyphenethyl)-6,9-dimethyl-1,5,8,11-tetraoxo-1,2,3,4,5,6,7,8,9,10,11,12,13,14-tetradecahydronaphtho[1,2-p][1]oxa[4,7,10,14]tetraazacycloheptadecine-3-carboxamide has the structure depicted in Table 3, wherein said compound is in absolute configuration as drawn:

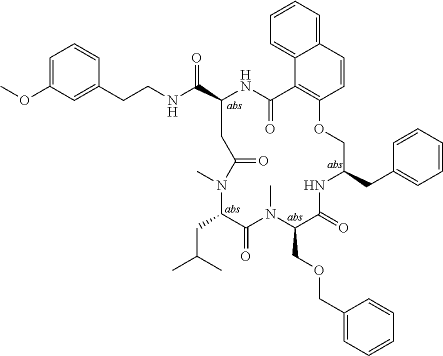 [SMILES: COc1cccc(CCNC(=O)[C@@H]2CC(=O)N(C)[C@@H](CC(C)C)C(=O)N(C)[C@H](COCc3ccccc3)C(=O)N[C@H](Cc3ccccc3)COc3ccc4ccccc4c3C(=O)N2)c1]

which is

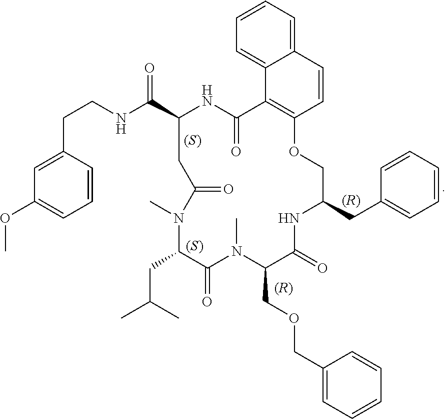

Likewise, the compound of example 724: (3R,6RS,9S,13S)-3-benzyl-6-((benzyloxy)methyl)-N-(2-(3-cyclopropyl-1,2,4-oxadiazol-5-yl)ethyl)-9-isobutyl-16-methoxy-7,10-dimethyl-5,8,11,15-tetraoxo-2,3,4,5,6,7,8,9,10,11,12,13,14,15-tetradecahydropyrido[3,4-p][1]oxa[4,7,10,14]tetraazacycloheptadecine-13-carboxamide has the structure depicted in Table 3, wherein, regarding the chiral centers at carbon atoms 3, 9, and 13, said compound is in absolute configuration as drawn; and with regard to the chiral center at carbon atom 6, the absolute configuration of said chiral center (marked as &1) may be both (R) or (S):

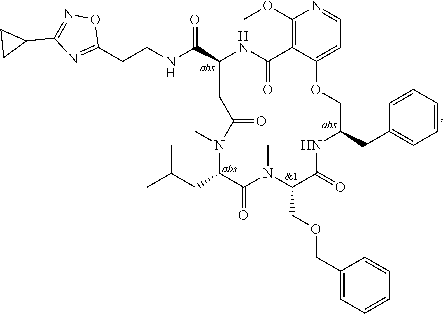 [SMILES: COc1nccc2c1C(=O)N[C@H](C(=O)NCCc1nc(C3CC3)no1)CC(=O)N(C)[C@@H](CC(C)C)C(=O)N(C)[C@@H](COCc1ccccc1)C(=O)N[C@H](Cc1ccccc1)CO2]

such compound encompassing the diastereomers:

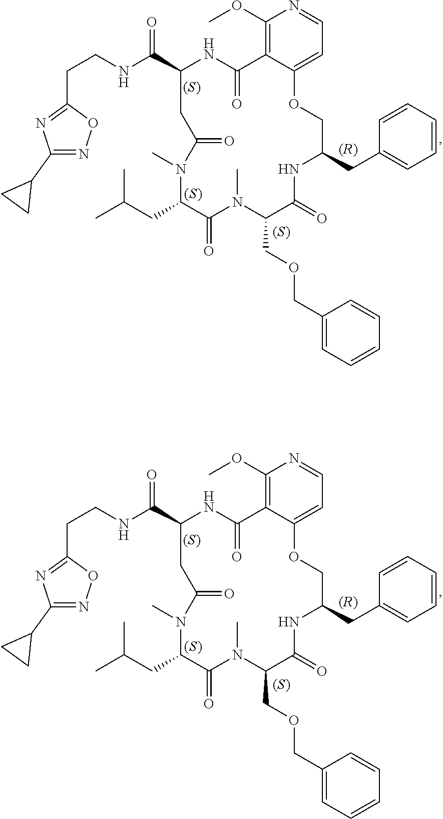

and any mixture thereof.

Likewise, the compound of example 769: (9S,13S,19aR,22R)-22-benzyl-5-fluoro-13-isobutyl-N-(2-(3-methoxyisoxazol-5-yl)ethyl)-12-methyl-7,11,14,20-tetraoxo-7,8,9,10,11,12,13,14,17,18,19,19a,20,21,22,23-hexadecahydro-16H-pyrido[2′,1′:6,7][1]oxa[4,7,10,14]tetraazacycloheptadecino[16,17-f]quinoline-9-carboxamide has the structure depicted in Table 4, wherein said compound is in absolute configuration as drawn:

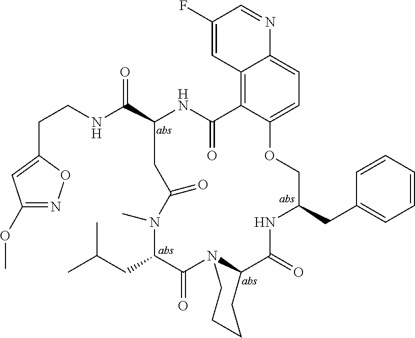

which is

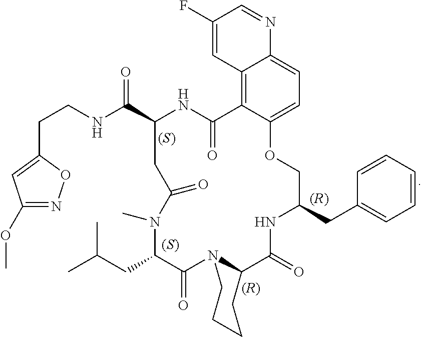

Likewise, the compound of example 820: (3R,6R,9S,13S)-3-benzyl-N-(2-(5-cyclopropyl-1,2,4-oxadiazol-3-yl)ethyl)-9-isobutyl-16-methoxy-7,10,18-trimethyl-5,8,11,15-tetraoxo-6-((3-(trifluoromethyl)-1,2,4-oxadiazol-5-yl)methyl)-2,3,4,5,6,7,8,9,10,11,12,13,14,15-tetradecahydropyrido[3,4-p][1]oxa[4,7,10,14]tetraazacycloheptadecine-13-carboxamide has the structure depicted in Table 4, wherein said compound is in absolute configuration as drawn:

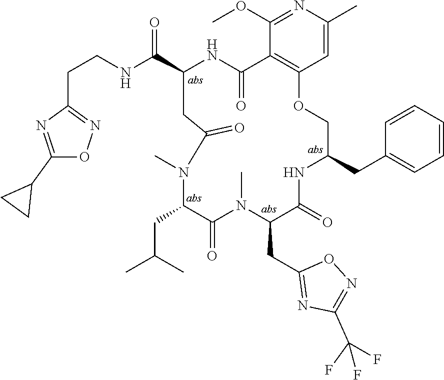

which is

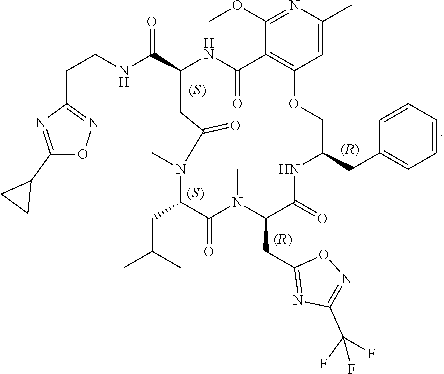

The compounds of formula (I)/formula (II) according to embodiments 1) to 21) and their pharmaceutically acceptable salts can be used as medicaments, e.g. in the form of pharmaceutical compositions for enteral (such especially oral e.g. in form of a tablet or a capsule) or parenteral administration (including topical application or inhalation).

The production of the pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the described compounds of formula (I)/formula (II) or their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

The present invention also relates to a method for the prevention/prophylaxis or treatment of a disease or disorder mentioned herein comprising administering to a subject a pharmaceutically active amount of a compound of formula (I)/formula (II) according to embodiments 1) to 21).

The compounds of formula (I)/formula (II) according to embodiments 1) to 21) are useful for the treatment of CFTR-related diseases and disorders, especially cystic fibrosis.

CFTR-related diseases and disorders may be defined as including especially cystic fibrosis, as well as further CFTR-related diseases and disorders selected from:
chronic bronchitis; rhinosinusitis; constipation; pancreatitis; pancreatic insufficiency; male infertility caused by congenital bilateral absence of the vas deferens (CBAVD); mild pulmonary disease; allergic bronchopulmonary aspergillosis (ABPA); liver disease; coagulation-fibrinolysis deficiencies, such as protein C deficiency; and diabetes mellitus;
asthma; COPD; smoke induced COPD; and dry-eye disease; and
idiopathic pancreatitis; hereditary emphysema; hereditary hemochromatosis; lysosomal storage diseases such as especially I-cell disease pseudo-Hurler; mucopolysaccharidoses; Sandhoff/Tay-Sachs; osteogenesis imperfecta; Fabry disease; Sjogren's disease; osteoporosis; osteopenia; bone healing and bone growth (including bone repair, bone regeneration, reducing bone resorption and increasing bone deposition); chloride channelopathies, such as myotonia congenita (Thomson and Becker forms); Bartter's syndrome type 3; epilepsy; lysosomal storage disease; Primary Ciliary Dyskinesia (PCD)—a term for inherited disorders of the structure and or function of cilia (including PCD with situs inversus also known as Kartagener syndrome, PCD without situs inversus, and ciliary aplasia); generalized epilepsy with fibrile seizures plus (GEFS+); general epilepsy with febrile and afebrile seizures; myotonia; paramyotonia congenital; potassium-aggravated myotonia; hyperkalemic periodic paralysis; long QT syndrome (LQTS); LQTS/Brugada syndrome; autosomal-dominant LQTS with deafness; autosomal-recessive LQTS; LQTS with dysmorphic features; congenital and acquired LQTS; dilated cardiomyopathy; autosomal-dominant LQTS; osteopetrosis; and Bartter syndrome type 3.

The term “treatment of cystic fibrosis” refers to any treatment of cystic fibrosis and includes especially treatment that reduces the severity of cystic fibrosis and/or reduces the symptoms of cystic fibrosis.

The term “cystic fibrosis” refers to any form of cystic fibrosis, especially to a cystic fibrosis that is associated with one or more gene mutation(s). Preferably, such cystic fibrosis is associated with an CFTR trafficking defect (class II mutations) or reduced CFTR stability (class VI mutations) [in particular, an CFTR trafficking defect/class II mutation], wherein it is understood that such CFTR trafficking defect or reduced CFTR stability may be associated with another disease causing mutation of the same or any other class. Such further disease causing CFTR gene mutation comprises class I mutations (no functional CFTR protein), (a further) class II mutation (CFTR trafficking defect), class III mutations (CFTR regulation defect), class IV mutations (CFTR conductance defect), class V mutations (less CFTR protein due to splicing defects), and/or (a further) class VI mutation (less CFTR protein due to reduced CFTR stability). Said one or more gene mutation(s) may for example comprise at least one mutation selected from F508del, A561E, and N1303K, as well as 1507del, R560T, R1066C and V520F; in particular F508del. In addition to the above-listed, further CFTR gene mutations comprise for example G85E, R347P, L206W, and M1101K. Said gene mutation(s) may be heterozygous, homozygous or compound hetereozygous. Especially said gene mutation is heterozygous comprising one F508del mutation. Further CFTR gene mutations (which are especially class Ill and/or IV mutations) comprise G551D, R117H, D1152H, A455E, S549N, R347H, S945L, and R117C.

The severity of cystic fibrosis/of a certain gene mutation associated with cystic fibrosis as well as the efficacy of correction thereof may generally be measured by testing the chloride transport effected by the CFTR. In patients, for example average sweat chloride content may be used for such assessment.

The term “symptoms of cystic fibrosis” refers especially to elevated chloride concentration in the sweat; symptoms of cystic fibrosis further comprise chronic bronchitis; rhinosinusitis; constipation; pancreatitis; pancreatic insufficiency; male infertility caused by congenital bilateral absence of the vas deferens (CBAVD); mild pulmonary disease; allergic bronchopulmonary aspergillosis (ABPA); liver disease; coagulation-fibrinolysis deficiencies such as protein C deficiency; and/or diabetes mellitus.

For avoidance of any doubt, if compounds are described as useful for the treatment of certain diseases, such compounds are likewise suitable for use in the preparation of a medicament for the treatment of said diseases. Likewise, such compounds are also suitable in a method for the treatment of such diseases, comprising administering to a subject in need thereof, an effective amount of such compound.

The term “subject” as used herein refers to a mammal, especially a human.

The present invention further relates to a method of treating cystic fibrosis, comprising the administration of an effective amount of a macrocycle (especially of a 17- or 18-membered macrocycle), or of a pharmaceutically acceptable salt thereof; to a subject in need thereof; wherein the cyclic core of said macrocycle comprises one aromatic moiety (such as an arylene or 5- to 10-membered heteroarylene, wherein said aromatic moiety especially is bound to the rest of the molecule/the ring members of said macrocycle (i) through a carbonyl group and (ii) through an oxygen atom, wherein notably said carbonyl group and said oxygen atom are attached to said aromatic moiety in a 1,2-diyl, or in a 1,3-diyl relationship), at least one beta-amino acid (wherein especially said beta-amino acid is bound through its amino group to the carbonyl group attached to said aromatic moiety), and at least one N-alkylated alpha-amino acid (wherein especially said N-alkylated alpha-amino acid is bound through its N-alkylated amino group to the carbonyl group of said beta-amino acid, and wherein notably such alpha-amino acid is glycine or a natural or non-natural amino acid bearing a hydrocarbon substituent); wherein said macrocycle is a corrector of a class II mutation of human CFTR (wherein especially folding, stability, degradation and/or trafficking of said CFTR, in particular of human F508del-CFTR, is corrected), wherein preferably the activity of said CFTR is corrected with at least the same efficacy as can be achieved with lumacaftor (wherein said activity/efficacy may be tested according to the method disclosed in the experimental part hereinafter).

Besides, any preferences and (sub-)embodiments indicated for the compounds of formula (II) (whether for the compounds themselves, salts thereof, compositions containing the compounds or salts thereof, or uses of the compounds or salts thereof, etc.) apply mutatis mutandis to compounds of formula (I).

Preparation of Compounds of Formula (I)/Formula (II):

The compounds of formula (I), formula (II), formula (I_{E}), formula (II_{E}) can be prepared by well-known literature methods, by the methods given below, by the methods given in the experimental part below or by analogous methods. Optimum reaction conditions may vary with the particular reactants or solvents used, but such conditions can be determined by a person skilled in the art by routine optimisation procedures. In some cases, the order of carrying out the following reaction schemes, and/or reaction steps, may be varied to facilitate the reaction or to avoid unwanted reaction products. In the general sequence of reactions outlined below, the generic groups R¹, R², R³, R⁴, Ar¹ and Ar² are as defined for formula (I), formula (II), formula (I_{E}), formula (II_{E}). Other abbreviations used herein are explicitly defined, or are as defined in the experimental section. In some instances, the generic groups R¹, R², R³, R⁴, Ar¹ and Ar² might be incompatible with the assembly illustrated in the schemes below and so will require the use of protecting groups (PG). The use of protecting groups is well known in the art (see for example “Protective Groups in Org. Synthesis”, T. W. Greene, P. G. M. Wuts, Wiley-Interscience, 1999). For the purposes of this discussion, it will be assumed that such protecting groups as necessary are in place. In some cases, the final product may be further modified, for example, by manipulation of substituents to give a new final product. These manipulations may include, but are not limited to, reduction, oxidation, alkylation, acylation, hydrolysis and transition-metal catalysed cross-coupling reactions which are commonly known to those skilled in the art. The compounds obtained may also be converted into salts, especially pharmaceutically acceptable salts, in a manner known per se.

Compounds of formula (I), formula (II), formula (I_{E}), and formula (II_{E}) of the present invention can be prepared according to the general sequence of reactions outlined below.

Compounds of formula (I) are prepared following one of the schemes depicted below.

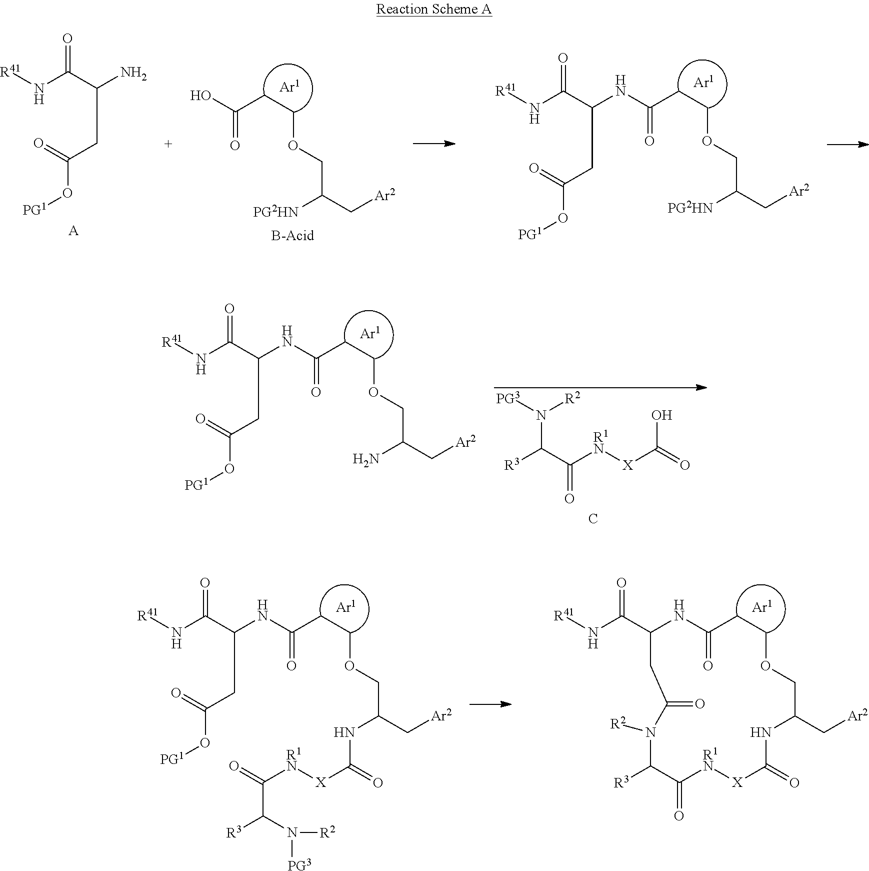

Reaction Scheme A: Syntheses can be performed with racemic or enantiomerically enriched amino acid building blocks. Suitably protected amine building block A and acid B-Acid, prepared following procedures well described in the literature or in Reaction Schemes I and J respectively, are treated with a peptide coupling reagent such as HATU, COMU, T3P, PyBop or EDCI/HOBt in a solvent like THF, DMF or NMP in the presence of a base such as TEA or DIPEA at a temperature between −20° C. and +75° C., preferably at RT, to generate the corresponding amide intermediate AB. Deprotection of the amine function of the intermediate AB is achieved according to known methodologies by those skilled in the art, e.g. by treatment with 4M HCl in dioxane or preferably with TFA in the case of a Boc protecting group, or with piperidine or diethylamine in the case of an Fmoc protecting group, or the appropriate treatment in case of other protecting groups such as Cbz or Alloc protecting groups. The deprotected intermediate AB-Amine is then reacted with the suitably protected acid C, prepared following procedures described in the literature or in the experimental section, according to the peptide coupling conditions already described above for the formation of the AB intermediate. The obtained linear intermediate ABC is then deprotected before the final peptide coupling macrolactamisation. In some cases, the protecting groups PG1 and PG3 are sequentially removed, but they are preferably removed simultaneously in one single step. For example, a tBu ester and Boc protecting groups are removed by treatment with 4M HCl in dioxane or preferably TFA, or alternatively Allyl ester and Alloc protecting groups can be removed by palladium catalyst treatment as extensively reported in the literature. The linear ABC deprotected intermediate is then cylised under standard conditions, i.e. the intermediate can be treated with a coupling reagent such as COMU, T3P, PyBop, EDCI/HOBt, or preferably HATU in diluted conditions such as less than 0.1M soln. of the ABC starting material in a solvent like DMF or NMP or a mix. of solvents like DMF/DCM (1:1), in presence of a base such as TEA or DIPEA at a temperature between −20° C. and +75° C., preferably at RT to yield the corresponding macrocycle cABC. Depending on the nature of the different residues some remaining deprotection steps may be required to yield the final product. Final purification by preparative HPLC, with standard reverse phase or if required, chiral phase columns gives the target compound as a pure stereoisomer.

Reaction Scheme B: In a modified version of Reaction Scheme A, the C moiety can be introduced stepwise, one amino acid at a time. The AB intermediate previously described in Reaction Scheme A, and the first amino acid D-1, commercially available or prepared following a procedure described in the literature, or in the experimental section below, are treated according to the peptide coupling conditions already described above to form the corresponding peptide bond. Selective deprotection of the amine function of ABD-1, such as removing an Fmoc group by treatment with piperidine or diethylamine, or removing a Cbz protecting group by hydrogenolysis over a catalyst such as Pd/C or Pd(OH)₂/C in a solvent like EtOAc, THF or dioxane, or preferably removing a Boc protecting group by treatment with 4M HCl in dioxane or with TFA, affords the free amine or its ammonium salt respectively, ready to be coupled with the second amino acid D-2 in a similar peptide coupling step. The three described coupling/deprotection/coupling steps yield the same linear intermediate ABC as the one previously described in Reaction Scheme A. The remaining steps of the synthesis to furnish the desired macrocycle cABC are the same as described above.

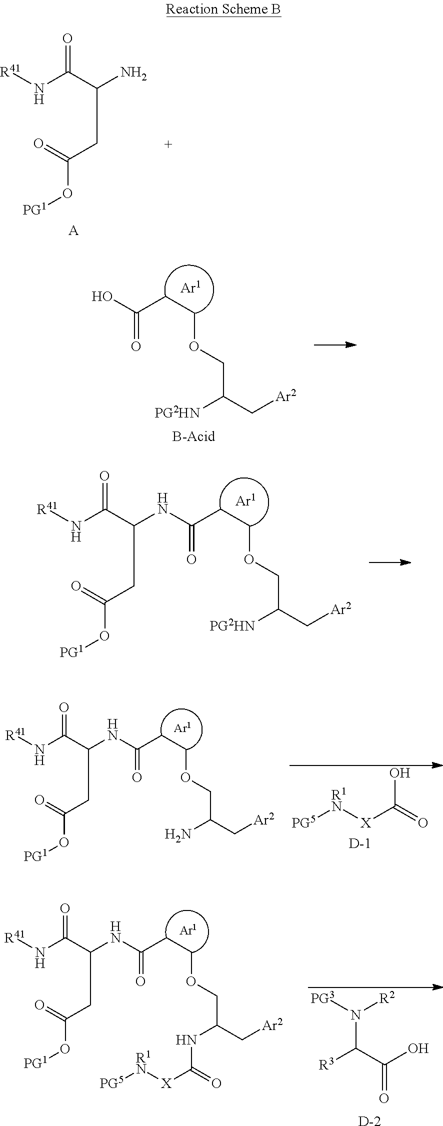

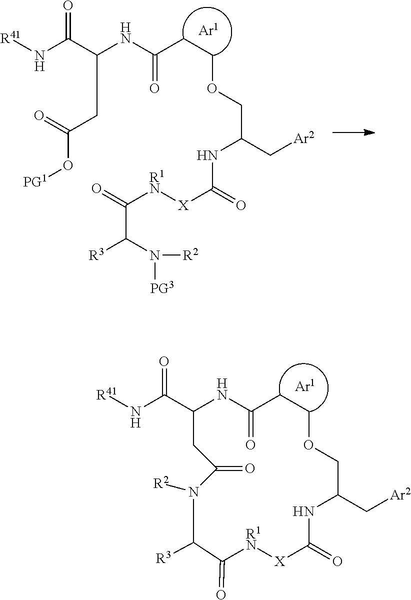

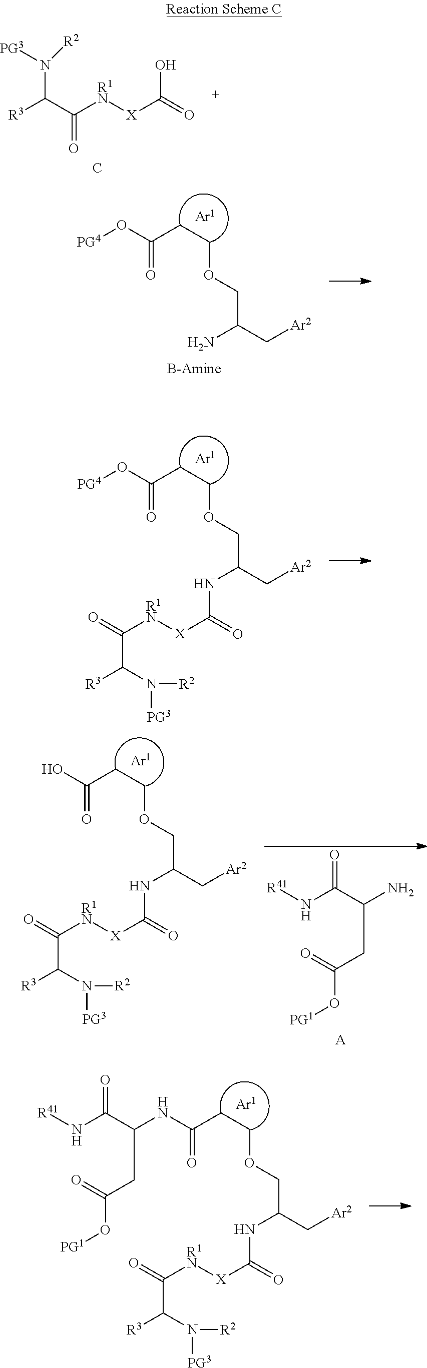

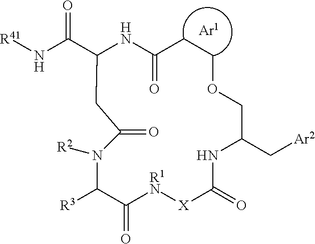

Reaction Scheme C: In an alternative approach, the sequence for building the linear intermediate ABC can be modified. Suitably protected building block C and the amine B-Amine, prepared following procedures described in the literature or in Reaction Schemes K and J respectively, are treated according to the peptide coupling conditions already described above, with a reagent such as HATU, COMU, T3P, PyBop or EDCI/HOBt in a solvent like THF, DMF or NMP in the presence of a base such as TEA or DIPEA at a temperature between −20° C. and +75° C., preferably at RT. Deprotection of the acid function of the intermediate BC, i.e. removal of PG4, is achieved according to known methodologies by those skilled in the art, e.g. by treatment with NaOH or LiOH in aqueous methanol at a temperature ranging from 0° C. up to 50° C. for methyl or ethyl esters or preferably by hydrogenolysis over a catalyst such as Pd/C or Pd(OH)₂/C in a solvent like EtOAc, THF or dioxane for benzyl esters. The deprotected intermediate BC-Acid is then reacted with the suitably protected amine building block A, prepared following procedures described in the literature or in Reaction Scheme I according to the peptide coupling conditions already described previously. The resulting linear ABC can then be deprotected and cyclised to yield the final product cABC as described in Reaction Scheme A.

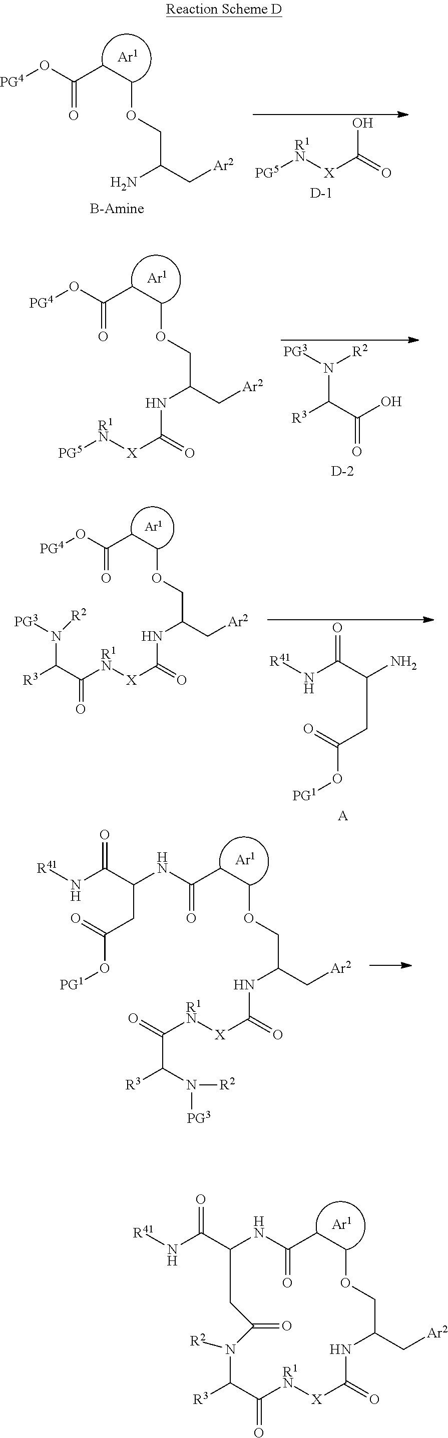

Reaction Scheme D: As it is the case when moving from Reaction Scheme A to Reaction Scheme B, the C moiety in Reaction Scheme C can be similarly introduced stepwise, one amino acid at a time. Suitably protected acid D-1 and the amine B-Amine, prepared following procedures described in the literature, or in the experimental part, or Reaction Scheme J, are treated according to the peptide coupling conditions already described above. Selective deprotection of the amine function of BD-1, i.e. removal of PG5, such as removing a Cbz protecting group under acidic conditions or more preferable, removing a Boc protecting group by treatment with 4M HCl in dioxane or preferably with TFA, affords the corresponding ammonium salt without removal of the orthogonal protecting group PG4. The resulting intermediate amine can then be coupled with the second amino acid D-2 in a similar peptide coupling step. The three described coupling/deprotection/coupling steps yield the same protected intermediate BC as the one previously described in Reaction Scheme C at which stage the rest of the synthesis can be performed as described above.

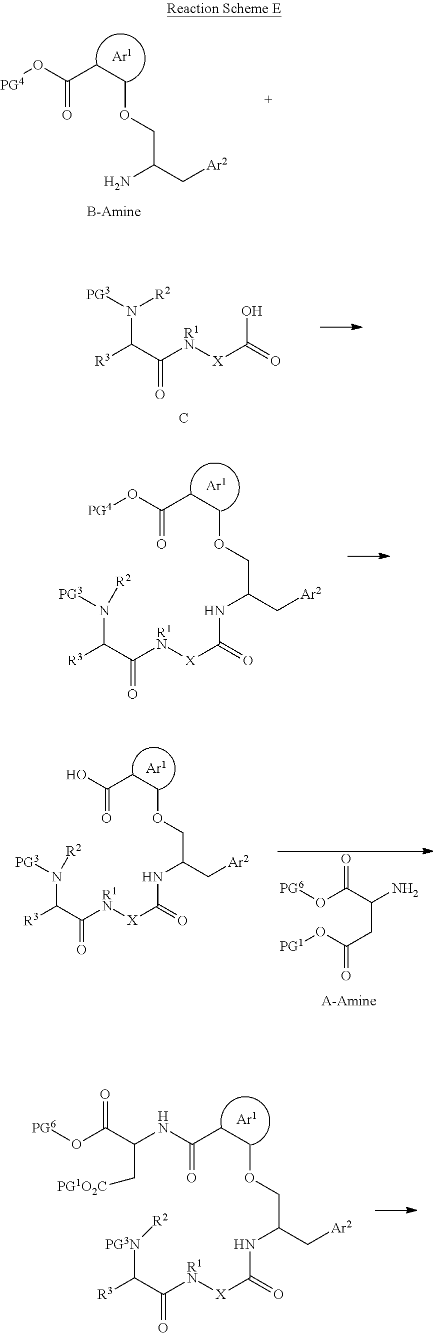

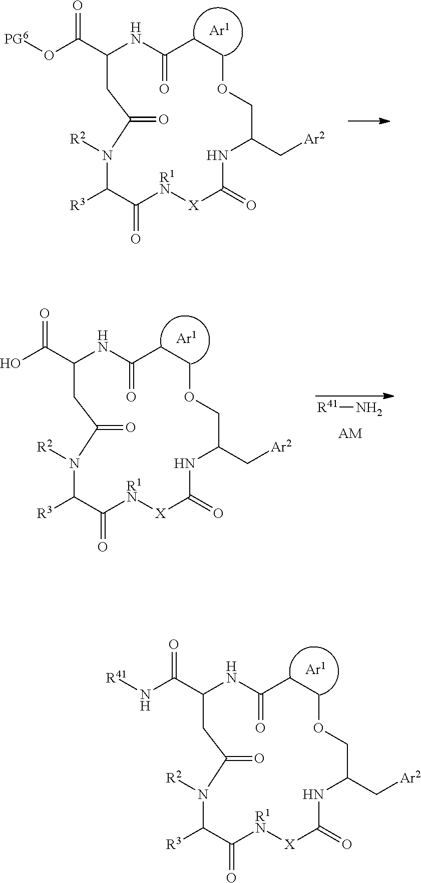

Reaction Scheme E: In another variation of Reaction Scheme C, the building block A-Amine is doubly protected with suitable orthogonal protecting groups on the 2 carboxylic acid functions, such as the a-benzyl ester or a-methyl ester in the presence of a β-butyl ester. Following the sequence described in Reaction Scheme C then yields the corresponding linear intermediate ABC. Double deprotection of the aspartic acid side chain and the Boc amine using TFA and subsequent cyclisation by a method already described previously yields the cyclised intermediate cABC, still protected on A. Deprotection of the aspartic acid backbone carboxylic acid, i.e. removal of PG6, can be accomplished by treatment with NaOH or LiOH in methanol/water at a temperature ranging from 0° C. to 50° C. for methyl or ethyl esters or preferably by hydrogenolysis of the benzyl ester over a catalyst such as Pd/C or Pd(OH)₂/C in a solvent like EtOAc, THF or dioxane. The deprotected intermediate cABC-Acid is then coupled according to peptide coupling conditions already described above with an amine AM, either commercially available or prepared following a procedure described in the literature or in the experimental section to yield the target compound. This strategy is especially efficient for preparation of libraries for the exploration of the AM moiety.

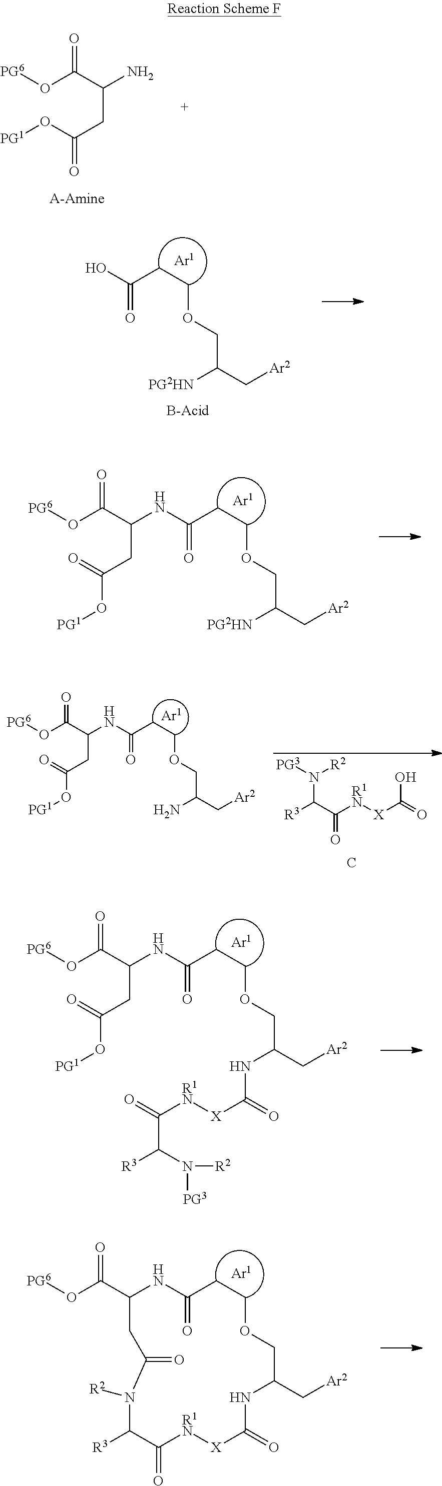

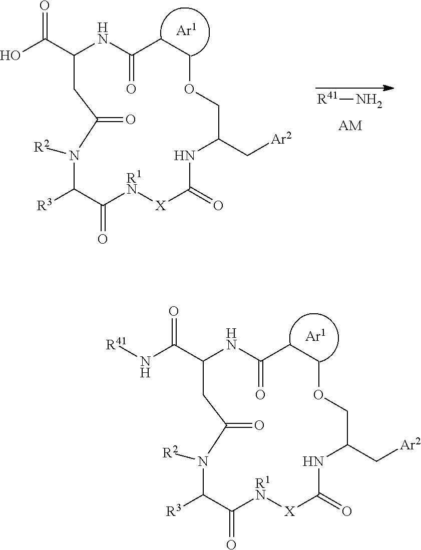

Reaction Scheme F: The strategy described in Reaction Scheme E, introducing the A moiety stepwise, can be applied in a different sequence to give the same cABC-Acid intermediate, as illustrated in Reaction Scheme F. The protected A-Amine, doubly protected with suitable orthogonal protecting groups on the 2 carboxylic acid functions, such as the a-benzyl ester or a-methyl ester in the presence of a R-allyl ester can be coupled with the required B-Acid and C building blocks in the same sequence as described in Reaction Scheme A to furnish the corresponding linear intermediate ABC. Sequential deprotection of the amine protecting group PG3, using TFA in the case of a Boc protecting group followed by removal of the aspartic acid side-chain protecting group PG1, by treatment with 1,3-dimethyl barbituric acid and Pd(PPh₃)₄ in a solvent like DCM in the case of an allyl protecting group, leaves only cyclisation as already described above to give the intermediate cABC, still protected on A as already described in Reaction Scheme E. The remaining steps of the synthesis to furnish the desired macrocycle cABC are the same as already described above.

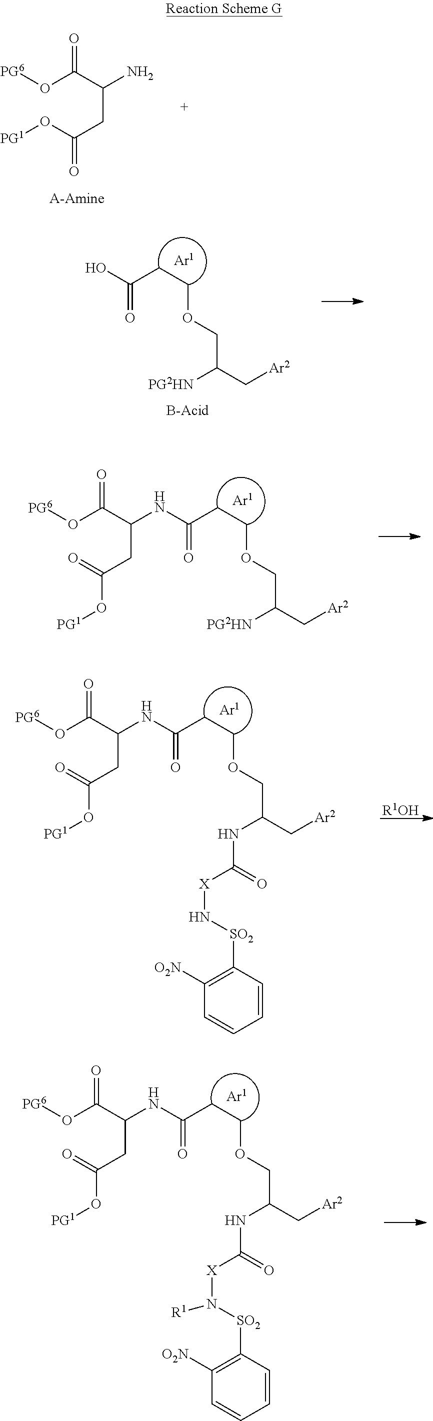

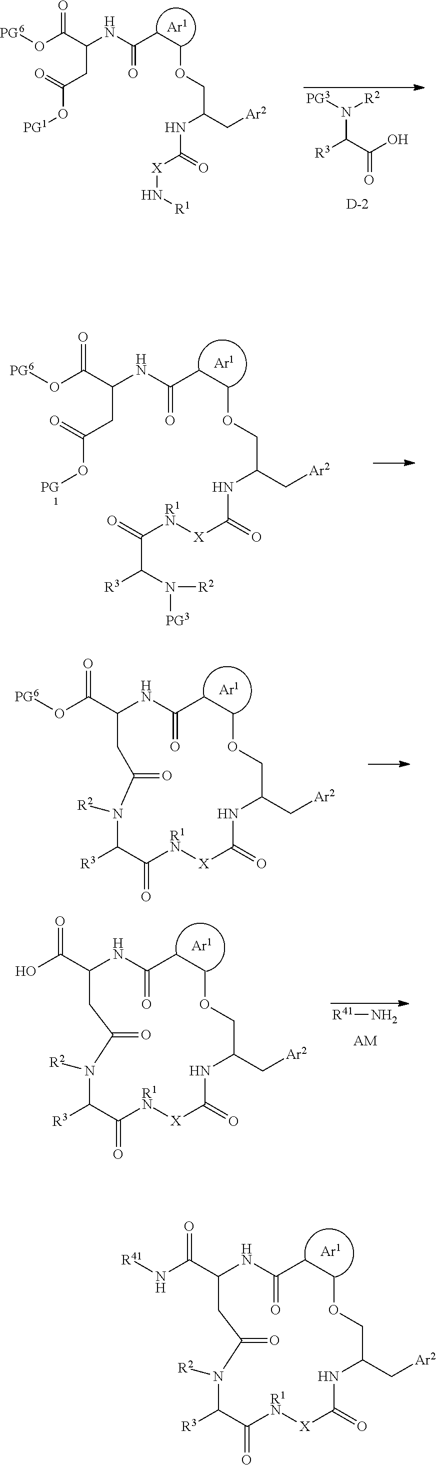

Reaction Scheme G: In a variation of Reaction Scheme F, and in close similarity to Reaction Schemes B and D, moiety C can be introduced stepwise, one amino acid at a time. Moreover, the amino acid D-1 can itself be built stepwise, by introducing the desired side-chain R1 on an already assembled ABD1 precursor. The amine deprotected AB intermediate already described in Reaction Scheme F can be coupled with an unsubstituted amino acid precursor of D-1, such as the NH-Boc or preferably the NH-nosyl-amino acid according to already described peptide coupling conditions. The NH-Nosyl function can then either be alkylated by treatment with the desired alkyl halide such as the bromide or preferably iodide in the presence of a base, such as K₂CO₃ or preferably, via a Mitsunobu reaction with the desired alcohol, performed according to standard conditions well known to those skilled in the art, e.g. by treatment with DEAD or DIAD with a phosphine ligand like triphenylphosphine at a temperature ranging from −80° C. up to 60° C. in a solvent such as THF or dioxane. The Nosyl activating/protecting group can then be removed by standard treatment with thiophenol in the presence of a base such as K₂CO₃ in a solvent like DMF to afford the corresponding deprotected intermediate. The amino acid D-2 can be coupled to this intermediate according to the conditions illustrated in Reaction Scheme B. The three described coupling/deprotection/coupling steps yield the same deprotected linear intermediate ABC as the one previously described in Reaction Scheme F. The remaining steps of the synthesis to furnish the desired macrocycle cABC are the same as already described above.

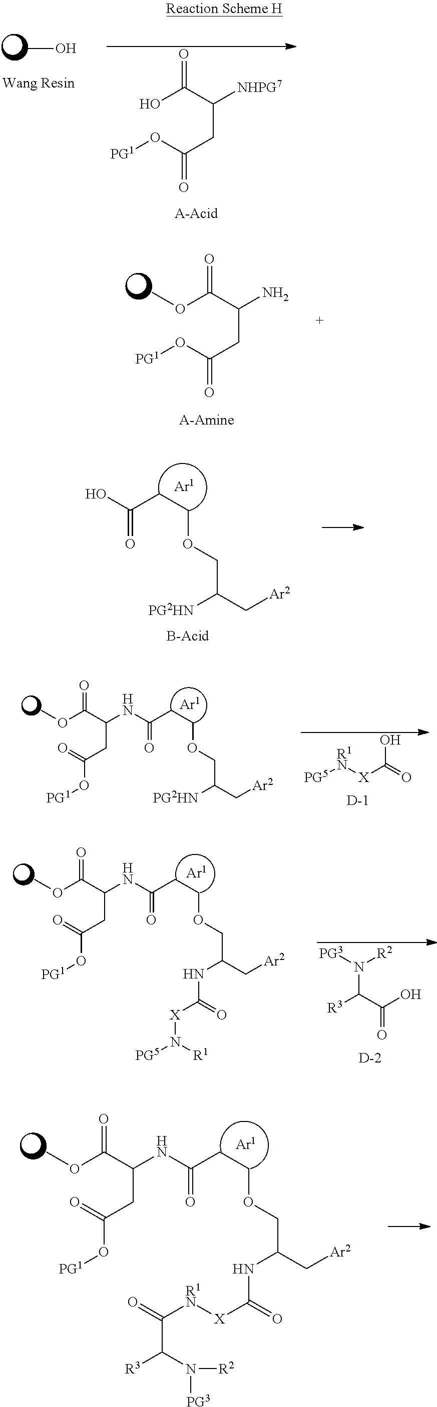

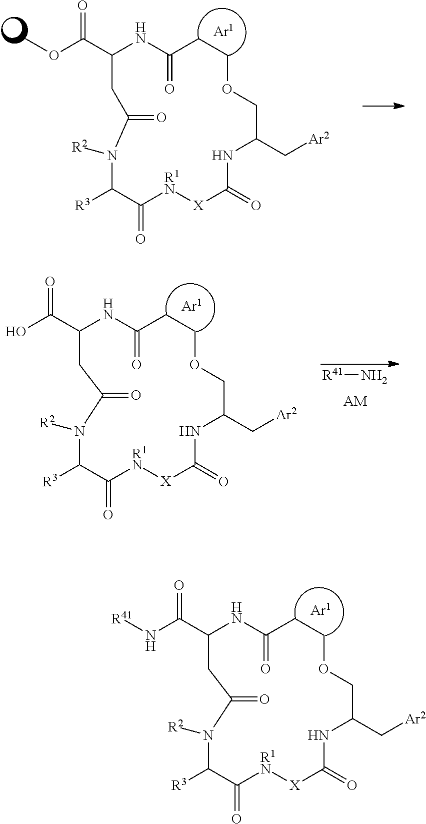

Reaction Scheme H: In a further adaptation of Reaction Scheme F, the a-carboxylic acid protecting group of the A-Amine building block can be solid phase such as a polymer-linked support, enabling the stepwise solid phase peptide synthesis of the cyclised macrocycle precursor according to established methodologies well known to those skilled in the art of polymer supported peptide synthesis. For instance, the amino acid A-Acid, suitably orthogonally protected on the amine function by for example an Fmoc protecting group and on the P-carboxylic acid function by for example an allylester, can be introduced on Wang resin by treatment with HOBt and DMAP and a coupling reagent such as DCC or DIC in a solvent mix. such as DCM/DMF allowing suitable swelling of the polymer beads. The subsequent sequence of deprotection of the Fmoc protecting group followed by peptide coupling with standard conditions for polymer peptide synthesis allows the stepwise introduction of the different building blocks, B-Acid, D1 and finally a suitably protected D2, like for example alloc-protected D2 gives the polymer supported linear peptide ABC, analogous to the one described in Reaction Scheme F. Double deprotection of the allyl ester and the N-alloc protecting groups can be achieved by treatment with a palladium catalyst, potentially in the presence of 1,3-dimethylbarbituric acid to furnish the still supported linear peptide. Cyclisation under standard peptide coupling conditions can be accomplished in these circumstances without risk of oligomer formation. The macrocycle cABC-Acid already described in Reaction Scheme F can then be released from the polymer support by acidic treatment such as with a mix. of TFA/H₂O (95/5). The liberated cABC-Acid can then be coupled with the appropriate AM amine using coupling conditions as described above to furnish the target compound.

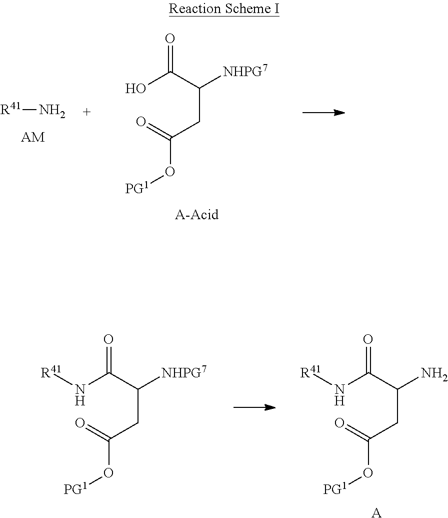

Building blocks A are either commercially available, prepared as described in the literature or may be prepared as illustrated in Reaction Scheme I. A suitably orthogonally protected A-Acid, such as the β-butylester of the N-Fmoc or the β-allylester of the N-Boc aspartic acid, is coupled with the desired AM amine according to standard peptide coupling conditions, by treatment with COMU or T3P, HATU, PyBop or another peptide coupling reagent, in a solvent like THF, DMF or NMP in the presence of a base such as TEA or DIPEA at a temperature between −20° C. and +75° C., preferably at RT. The resulting intermediate can then be selectively deprotected on the amine functionality without removing the 3-ester protecting group PG1, under standard conditions well established in the field of protecting group chemistry. Specific treatment with piperidine or diethylamine to remove the N-Fmoc in the presence of the β-butylester or with TFA or 4M HCl in dioxane to remove the N-Boc in the presence of the β-allylester gives access to the target building block A as its free base or its ammonium salt respectively.

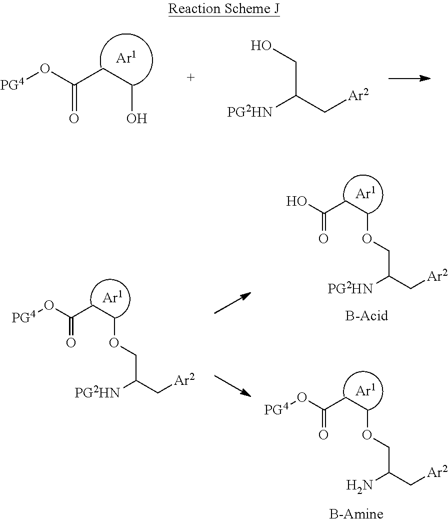

The building blocks B, B-Acid or B-Amine, are either prepared as described in the literature or may be prepared as illustrated in Reaction Scheme J. An appropriate salicylic acid derivative, protected as an ester on the carboxylic acid function, such as a methyl, ethyl, or benzyl ester, are either commercially available or prepared as described in the literature, or may be prepared as described in the experimental section. Similarly, the amino alcohol protected on the amine function by the Boc or Cbz groups are either commercially available or readily prepared from the corresponding amino acid, as described in the literature, or may also be prepared as described in the experimental section. The alcohol function of the amino alcohol can be activated upon treatment with methanesulfonyl chloride or toluenesulfonyl chloride or a similar activating agent, in the presence of a base such as DIPEA or TEA and reacted with the phenol function of the salicylic acid ester derivative in a solvent such as THF or DMF to furnish the doubly protected B building block. Alternatively, the two building blocks can be reacted together according to Mitsunobu methodology, by treatment with a phosphine ligand like triphenylphosphine and the DEAD or DIAD reagents in a solvent such as THF or dioxane at a temperature ranging from −20° C. up to 60° C. The resulting orthogonally protected intermediate can then be selectively deprotected on the acid function or on the amine function to access the corresponding building blocks B-Acid or B-Amine respectively. For example saponification of a methyl ester with aq. NaOH or LiOH soln. or hydrogenolysis of a benzyl ester over a palladium catalyst such as charcoal supported Pd or Pd(OH)₂ gives access to the corresponding B-Acid. Alternatively, Boc deprotection by treatment with TFA or hydrogenolysis of a Cbz protected amine in the case of a methyl ester leads to the corresponding B-Amine.

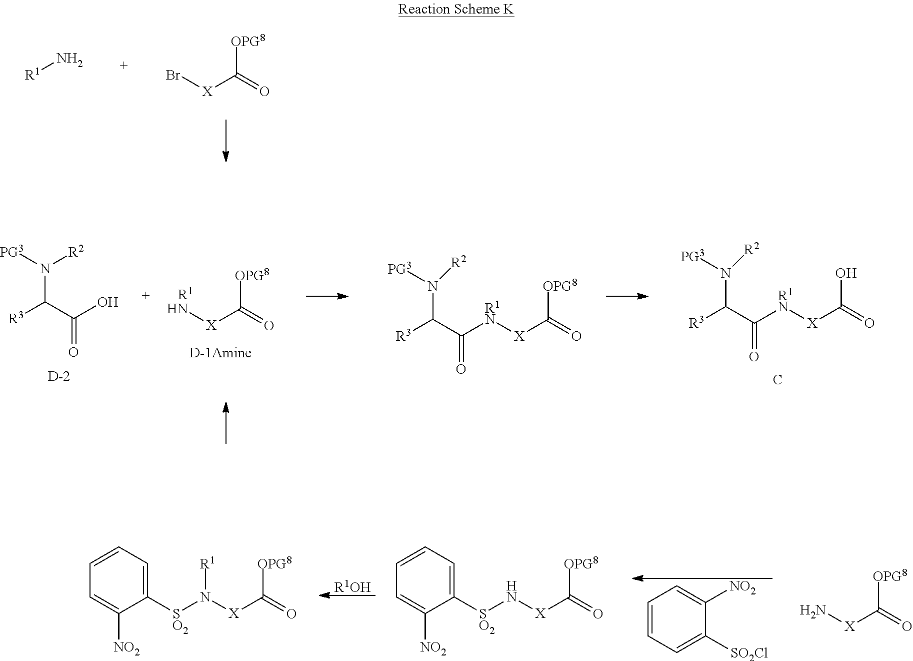

Building blocks C may be prepared as illustrated in Reaction Scheme K from the key intermediate D-1Amine. The intermediate D-1 is either commercially available or prepared as described in the literature or may be prepared as illustrated in this scheme. A suitably PG8 protected bromoacetic acid ester derivative, such as methyl, ethyl or benzyl ester, can be reacted with the appropriate amine R1NH₂, in a solvent like MeCN, acetone or DMF in the presence of a base such as K₂CO₃ or DIPEA at a temperature ranging from RT up to 80° C. to yield the amine D-1. Alternatively, a suitably PG8 protected amino acid ester derivative, such as methyl, ethyl or benzyl ester, can be reacted with nitrosulfonylbenzene chloride in the presence of a catalytic amount of DMAP, in a solvent such as DCM or THF to yield the corresponding N-Nosyl protected amine. Alkylation of the sulfonamide nitrogen can then be accomplished by Mitsunobu methodology as already described above, i.e. by reaction in the presence of the desired alcohol R10H with a phosphine ligand like triphenylphosphine and the DEAD or DIAD reagents, in a solvent such as THF or dioxane at a temperature ranging from 0° C. up to 80° C. Subsequent cleavage of the Nosyl group can be achieved by treatment with thiophenol in the presence of a base such as K₂CO₃ in a solvent like DMF or DCM to give the amine building block D-1. Coupling with the commercially available D-2 amino acid, or prepared as described in the literature, according to standard peptide coupling methodology as described above. Deprotection of the ester can then be accomplished by treatment with aq. NaOH or LiOH soln. in the case of a methyl or ethyl ester, or by hydrogenolysis of the benzyl ester over a palladium catalyst such as charcoal supported Pd or Pd(OH)₂ to yield the target C building block.

The following examples are provided to illustrate the invention. These examples are illustrative only and should not be construed as limiting the invention in any way.

### EXPERIMENTAL PART

I. Chemistry

All temperatures are stated in ° C. Commercially available starting materials were used as received without further purification. Unless otherwise specified, all reactions were carried out in oven-dried glassware under an atmosphere of nitrogen. Compounds were purified by flash column chromatography on silica gel or by preparative HPLC. Compounds described in the invention are characterised by LC-MS data (retention time t_{R} is given in min; molecular weight obtained from the mass spectrum is given in g/mol) using the conditions listed below. In cases where compounds of the present invention appear as a mix. of conformational isomers, particularly visible in their LC-MS spectra, the retention time of the most abundant conformer is given.

Analytical LC-MS Equipment:
HPLC pump: Binary gradient pump, Agilent G4220A or equivalent
Autosampler: Gilson LH215 (with Gilson 845z injector) or equivalent
Column compartment: Dionex TCC-3000RS or equivalent
Degasser: Dionex SRD-3200 or equivalent
Make-up pump: Dionex HPG-3200SD or equivalent
DAD detector: Agilent G4212A or equivalent
MS detector: Single quadrupole mass analyzer, Thermo Finnigan MSQPIus or equivalent
ELS detector: Sedere SEDEX 90 or equivalent

LC-MS with Acidic Conditions
Method A: Column: Zorbax SB-aq (3.5 μm, 4.6×50 mm). Conditions: MeCN [eluent A]; water+0.04% TFA [eluent B]. Gradient: 95% B→5% B over 1.5 min (flow: 4.5 mL/min). Detection: UV/Vis+MS.
Method B: Column: Zorbax RRHD SB-aq (1.8 μm, 2.1×50 mm). Conditions: MeCN [eluent A]; water+0.04% TFA [eluent B]. Gradient: 95% B→5% B over 2.0 min (flow: 0.8 mL/min). Detection: UV/Vis+MS.
Method C: Column: Waters XBridge C18 (5 μm, 4.6×30 mm). Conditions: MeCN [eluent A]; water+0.04% TFA [eluent B]. Gradient: 95% B→5% B over 1.5 min (flow: 4.5 mL/min). Detection: UV/Vis+MS.
Method D: Column: Waters BEH C18 (2.1×50 mm, 2.5 μm). Conditions: MeCN [eluent A]; water+0.04% TFA [eluent B]. Gradient: 95% B→5% B over 2.0 min (flow: 0.8 mL/min). Detection: UV/Vis+MS.
Method E: Column: Waters XBridge C18 (2.5 μm, 4.6×30 mm). Conditions: MeCN [eluent A]; water+0.04% TFA [eluent B]. Gradient: 95% B→5% B over 1.5 min (flow: 4.5 mL/min). Detection: UV/Vis+MS.
Method F: Column: Waters XSelect CSH C18 (3.5 μm, 2.1×30 mm). Conditions: MeCN+0.1% formic acid [eluent A]; water+0.1% formic acid [eluent B]. Gradient: 95% B→2% B over 1.6 min (flow 1 mL/min), Detection: UV/Vis+MS.
Method G: Column: Waters Atlantis T3 (3.0 μm, 2.1×50 mm). Conditions: MeCN+0.1% formic acid [eluent A]; water+0.1% formic acid [eluent B]. Gradient: 95% B→2% B over 5 min (flow 0.8 mL/min). Detection: UV/Vis+MS.
Method H: Waters Acquity Binary, Solvent Manager, MS: Waters SQ Detector or Xevo TQD or SYNAPT G2 MS, DAD: Acquity UPLC PDA Detector, ELSD: Acquity UPLC ELSD. Column ACQUITY UPLC CSH C18 1.7 um 2.1×50 mm from Waters, thermostated in the Acquity UPLC Column Manager at 60° C. Eluents: A: H₂O+0.05% formic acid; B: MeCN+0.045% formic acid. Method: Gradient: 2% B→98% B over 2.0 min. Flow: 1.0 mL/min. Detection: UV 214 nm and ELSD, and MS, t_{R} is given in min.

LC-MS with Basic Conditions
Method I: Column: Waters BEH C18 (2.5 μm, 2.1×50 mm). Conditions: water/NH₃ [c(NH₃)=13 mmol/l] [eluent A]; MeCN [eluent B]. Gradient: 5% B→95% B over 2 min (flow 0.8 mL/min). Detection: UV/Vis+MS.
Method J: Column: Waters XSelect CSH C18 (3.5 μm, 2.1×30 mm). Conditions: 95% MeCN+5% Water/NH₄HCO₃ [c(NH₄HCO₃)=10 mmol/l] [eluent A]; Water/NH₄HCO₃ [c(NH₄HCO₃)=10 mmol/l] [eluent B]. Gradient: 95% B→2% B over 1.6 min (flow 1 mL/min), Detection: UV/Vis+MS.

GC-MS
Agilent 6890N/Column: RXi-5MS 20m, ID 180 μm, df 0.18 μm; Velocity 50 cm/s, He carrier gas; 100° C.→250° C. over 4.5 min; Detection: MS.

Preparative HPLC Equipment:
Gilson 333/334 HPLC pump equipped with Gilson LH215, Dionex SRD-3200 degasser,
Dionex ISO-3100A make-up pump, Dionex DAD-3000 DAD detector, Single quadrupole mass analyzer MS detector, Thermo Finnigan MSQ Plus, MRA100-000 flow splitter, Polymer Laboratories PL-ELS1000 ELS detector

Preparative HPLC with Basic Conditions
Column: Waters XBridge (10 μm, 75×30 mm). Conditions: MeCN [eluent A]; water+0.5% NH₄OH (25% aq.) [eluent B]; Gradient see Prep. HPLC Table 1 (flow: 75 mL/min), the starting percentage of Eluent A (x) is determined depending on the polarity of the compound to purify. Detection: UV/Vis+MS

Prep. HPLC Table 1

| | t (min) | | | | | |
|---|---|---|---|---|---|---|
| | 0 | 0.01 | 4.0 | 6.0 | 6.2 | 6.6 |
| Eluent A (%) | x | x | 95 | 95 | x | x |
| Eluent B (%) | 100-x | 100-x | 5 | 5 | 100-x | 100-x |

Preparative HPLC with Acidic Conditions
Column: Waters Atlantis T3 (10 μm, 75×30 mm). Conditions: MeCN [eluent A]; water+0.5% HCO₂H [eluent B]; Gradient see Prep. HPLC Table 2 (flow: 75 mL/min), the starting percentage of Eluent A (x) is determined depending on the polarity of the compound to purify. Detection: UV/Vis+MS

Prep. HPLC Table 2

| | t (min) | | | | | |
|---|---|---|---|---|---|---|
| | 0 | 0.01 | 4.0 | 6.0 | 6.2 | 6.6 |
| Eluent A (%) | x | x | 95 | 95 | x | x |
| Eluent B (%) | 100-x | 100-x | 5 | 5 | 100-x | 100-x |

Preparative HPLC for Chiral Separations

In most cases, desired diastereoisomers can be isolated or purified by standard preparative scale HPLC according to standard methods well-known to those skilled in the art. In some instances, the use of a chiral chromatography column is advisable to separate complex mixtures of diastereoisomers. Best results are obtained using Chiral Stationary Phase columns, such as Chiralpak IA, IB, or IC columns based on an immobilised amylose or cellulose chiral phase, with an isocratic eluent based on a mix. of MeCN with EtOH or MeOH, in a ratio varying from 9:1 to 1:9. In order to compensate for the presence of ionisable functional groups in the compound being purified, modifiers can be added to the solvent mix. such as 0.1% diethylamine for basic derivatives or 0.1% formic acid for acidic ones. Supercritical Fluid Chromatography was used in some cases, using the same Chiral Stationary Phase columns as described above with isocratic eluents composed of 50% to 90% supercritical carbondioxide together with EtOH, MeOH or a 1:1 EtOH:MeCN mix. Detection: UV/Vis.

### Abbreviations (as Used Hereinbefore or Hereinafter)

AcOH acetic acid
Ac₂O acetic anhydride
Alloc allyloxycarbonyl
anh. anhydrous
aq. aqueous
atm atmosphere
BnBr benzyl bromide
Boc tert-butoxycarbonyl
Boc₂O di-tert-butyl dicarbonate
BOP (benzotriazol-1-yloxy)-tris(dimethylamino)-phosphonium hexafluorophosphateBuLi n-butyllithium
CDI 1,1′-carbonyldiimidazole
CD₃I iodomethane-d3
CHCl₃ chloroform
COMU (1-Cyano-2-ethoxy-2-oxoethylidenaminooxy)dimethylamino-morpholino-carbenium hexafluorophosphate
Cu(OAc)₂ copper (II) acetate
d days
DBU 1,8-diazabicyclo[5.4.0]undec-7-ene
DCC N,N′-dicyclohexylcarbodiimide
DCE 1,2-dichloroethane
DCM dichloromethane
DEAD diethyl azodicarboxylate
DIAD diisopropyl azodicarboxylate
DIBAL/DIBAL-H diisobutylaluminium hydride
DIC N,N′-diisopropylcarbodiimide
DIPEA diisopropyl-ethylamine, Hunig's base
DMAP 4-Dimethylaminopyridine
DMF dimethylformamide
DMSO dimethylsulfoxide
DPPA diphenyl phosphorylazide
dppf 1,1′-bis(diphenylphosphino)ferrocene
EDC 1-ethyl-3-(3-dimethylaminopropyl)carbodiimide
Et ethyl
Et₂O diethylether
EtOAc ethyl acetate
EtOH ethanol
evaporated evaporated in vacuo
Ex. example
FC flash chromatography on silica gel
FDPP pentafluorophenyl diphenylphosphinate
Fmoc 9-Fluorenylmethoxycarbonyl
h hour(s)
HATU (1-[Bis(dimethylamino)methylene]-1H-1,2,3-triazolo[4,5-b]pyridinium 3-oxid hexafluorophosphate
Hept heptane(s)
Hex hexane(s) HOBT 1-hydroxybenzotriazol
HPLC high performance liquid chromatography
HV high vacuum conditions
ⁱBu isobutyl
ⁱPr isopropyl
iPrOH isopropyl alcohol
ⁱPrOAc isopropyl acetate
KOtBu potassium tert-butoxide
LAH Lithium aluminium hydride
LC-MS liquid chromatography-mass spectrometry
Lit. Literature
M mol/l
mCPBA m-chloroperoxybenzoic acid
Me methyl
MeCN acetonitrile
Mel iodomethane
Meldrum's acid 2,2-Dimethyl-1,3-dioxane-4,6-dione
MeOH methanol
mL milliliter
min minute(s)
mix. mixture
MOM methoxymethyl
MW microwave
NaBH(OAc)₃ sodium triacetoxyborohydrideNCS N-chlorosuccinimideNMP N-methyl-2-pyrrolidone
nosyl 4-Nitrobenzenesulfonyl
ⁿPr n-propyl
OAc acetate
org. organic
Pd(^{t}Bu₃P)₂ bis(tri-tert-butylphosphine)palladium(0)
Pd(OAc)₂ palladium(II) acetate
Pd/C palladium on activated charcoal
Pd(OH)₂/C palladium hydroxide on activated charcoal (Pearlman's catalyst)
Pd₂(dba)₃ tris(dibenzylideneacetone)dipalladium(0)
PdCl₂(PPh₃)₂ bis(triphenylphosphine)palladium(II) dichloride
Pd(dppf)Cl₂ [1,1′-bis(diphenylphosphino)ferrocene]dichloropalladium(II)
Pd(dppf)Cl₂.DCM [1,1′-bis(diphenylphosphino)-ferrocene]dichloropalladium (II) complex with dichloromethane
Pd(PPh₃)₄ tetrakis(triphenylphosphine)palladium(0)
Ph phenyl
PhMe toluene
PPh₃ triphenyl phosphine
prep. preparative
PTFE polytetrafluoroethylene
PyBOP (benzotriazol-1-yl-oxy)-tripyrrolidino-phosphonium hexafluorophosphate
PyClop Chlorotripyrrolidinophosphonium hexafluorophosphate
rac racemic
RM reaction mix.
Rochelle's salt sodium potassium tartrate
RT room temperature
RuPhos 2-dicyclohexylphosphino-2′,6′-diisopropoxybiphenyl
s second(s)
sat. saturated
Selectfluor 1-chloromethyl-4-fluoro-1,4-diazoniabicyclo[2.2.2]octane bis(tetrafluoroborate)
SM starting material
soln. solution
TBAF tetrabutylammonium fluoride
TBDMSCl tert-butyldimethylsilyl chloride
TBME tert-butyl methyl ether
tBu tert-butyl=tertiary butyl
TEA triethylamine
Tf trifluoromethanesulfonyl
TFA trifluoroacetic acid
THF tetrahydrofuran
TLC thin layer chromatography
TMEDA N,N,N′,N′-tetramethylethylenediamine
TMS trimethylsilyl
tosyl p-toluene-sulfonyl
T₃P n-propylphosphonic anhydride
t_{R} retention time
triflate trifluoromethanesulfonate
pTsOH p-toluenesulfonic acid
Xantphos 4,5-bis(diphenylphosphino)-9,9-dimethylxanthene
XPhos 2-dicyclohexylphosphin-2′,4′,6′-triisopropylbiphenyl

A—Preparation of Precursors and Intermediates

Amines:

Commercially available amines are depicted in Table AM-1.

TABLE AM-1

| Amine | Name |
|---|---|
| AM-1.1 | 3,4-Methylenedioxyphenethylamine•HCl |
| AM-1.2 | 2-(1,3-Dihydro-isobenzofuran-5-yl)-ethylamine |
| AM-1.3 | 2-(1,3-Benzoxazol-2-yl)ethanamine•HCl |
| AM-1.4 | 3-Methoxyphenethylamine |
| AM-1.5 | 4-Methoxyphenethylamine |
| AM-1.6 | Phenethylamine |
| AM-1.7 | 3,5-Dimethoxyphenethylamine |
| AM-1.8 | 2-(p-tolyl)Ethylamine |
| AM-1.9 | 3-Methylphenethylamine |
| AM-1.10 | 4-Ethylphenethylamine |
| AM-1.11 | 2-(4-tert-Butylphenyl)ethylamine |
| AM-1.12 | 4-Bromophenethylamine |
| AM-1.13 | 2-Fluorophenethylamine |
| AM-1.14 | 2-(3-Chlorophenyl)ethylamine |
| AM-1.15 | 3-Bromophenethylamine |
| AM-1.16 | 3,4-Dimethylphenethylamine |
| AM-1.17 | 2,4-Dimethylphenethylamine |
| AM-1.18 | 2-Methylphenethylamine |
| AM-1.19 | 2,3-Dimethoxyphenethylamine |
| AM-1.20 | 3-Fluorophenethylamine |
| AM-1.21 | 4-Fluorophenethylamine |
| AM-1.22 | 2-(2,6-Difluorophenyl)ethan-1-amine |
| AM-1.23 | 2-(3-Phenyl-[1,2,4]oxadiazol-5-yl)-ethylamine•HCl |
| AM-1.24 | [2-(5-Methyl-1,2,4-oxadiazol-3-yl)ethyl]amine•HCl |
| AM-1.25 | [2-(5-Ethyl-1,2,4-oxadiazol-3-yl)ethyl]amine•HCl |
| AM-1.26 | 2-(5-Cyclopropyl-1,2,4-oxadiazol-3-yl)ethan-1-amine•HCl |
| AM-1.27 | 2-(5-Methyl-1,2-oxazol-4-yl)ethan-1-amine•HCl |
| AM-1.28 | 2-(1-Methyl-1H-pyrazol-4-yl)ethanamine |
| AM-1.29 | 2-(5-tert-Butyl-1,2,4-oxadiazol-3-yl)ethanamine•HCl |
| AM-1.30 | 2-(5-tert-Butyl-1,2-oxazol-3-yl)ethan-1-amine |
| AM-1.31 | 2-(5-Methyl-1,2-oxazol-3-yl)ethan-1-amine•HCl |
| AM-1.32 | 2-(1,2-Thiazol-5-yl)ethan-1-amine•HCl |
| AM-1.33 | 2-(5-Cyclopropyl-1,2-oxazol-3-yl)ethan-1-amine |
| AM-1.34 | 2-(3,5-Difluorophenyl)ethan-1-amine |
| AM-1.35 | 2-(3-Cyclopropyl-1,2,4-oxadiazol-5-yl)ethan-1-amine•HCl |
| AM-1.36 | [2-(5-Cyclobutyl-1,2,4-oxadiazol-3-yl)ethyl]amine•HCl |
| AM-1.37 | 2-(1,4-Dimethyl-1H-pyrazol-3-yl)ethan-1-amine |
| AM-1.38 | 2,2-Difluoro-2-phenylethanamine•HCl |
| AM-1.39 | 2-(3-Methyl-[1,2,4]oxadiazol-5-yl)-ethylamine•HCl |
| AM-1.40 | 2-(3-Methyl-1,2-oxazol-5-yl)ethan-1-amine•HCl |
| AM-1.41 | 2-[4-(Difluoromethoxy)phenyl]ethan-1-amine |
| AM-1.42 | 2-Pyrazin-2-yl-ethylamine |
| AM-1.43 | 2-Pyrimidine-4-yl-ethylamine•HCl |
| AM-1.44 | 2-(Pyrimidin-5-yl)ethan-1-amine•2HCl |
| AM-1.45 | 3-(Difluoromethoxy)propan-1-amine |
| AM-1.46 | 1-(2-Aminoethoxy)-2-methoxyethane |
| AM-1.47 | 2-(4-Methyl-1,3-thiazol-2-yl)ethan-1-amine |
| AM-1.48 | 2-(5-Methyl-1,3-thiazol-2-yl)ethan-1-amine |
| AM-1.49 | 2-{4H,5H,6H-Cyclopenta[d][1,3]thiazol-2-yl}ethan- |
| | 1-amine |
| AM-1.50 | 2-(1,2-Benzoxazol-3-yl)ethan-1-amine•HCl |
| AM-1.51 | 8-Methoxy-3,4-dihydro-2H-1-benzopyran-3-amine |
| AM-1.52 | 2-(3-Methoxyphenoxy)ethanamine |
| AM-1.53 | 2-(4-Methoxyphenoxy)ethanamine |
| AM-1.54 | 4-(2-Aminoethyl)benzonitrile•HCl |
| AM-1.55 | 2-(3,5-Dimethylisoxazol-4-yl)ethanamine |
| AM-1.56 | 2-(2-Isopropyl-1,3-thiazol-4-yl)ethanamine•HCl |
| AM-1.57 | 2-(3-(Difluoromethoxy)phenyl)ethan-1-amine |
| AM-1.58 | 2-(3-Ethoxyphenyl)ethan-1-amine |
| AM-1.59 | rac-2-(5,5-Dimethyltetrahydro-2H-pyran-2-yl)ethan- |
| | 1-amine |
| AM-1.60 | 4-(2-Aminoethyl)-N,N-dimethylaniline |
| AM-1.61 | 2-(Thiophen-2-yl)ethan-1-amine |
| AM-1.62 | 2-(5-Methoxypyridin-2-yl)ethan-1-amine |
| AM-1.63 | 2-(Furan-2-yl)ethan-1-amine |
| AM-1.64 | rac-2-(Tetrahydro-2H-pyran-2-yl)ethan-1-amine |
| AM-1.65 | 3-Methoxypropan-1-amine |
| AM-1.66 | 2-(3,5-Dimethyl-1H-pyrazol-1-yl)ethan-1-amine |
| AM-1.67 | rac-2-(2,3-Dihydrobenzo[b][1,4]dioxin-2-yl)ethan-1- |
| | amine•HCl |
| AM-1.68 | 5-Methoxypentan-1-amine |
| AM-1.69 | 2-(4-(1H-Pyrazol-1-yl)phenyl)ethan-1-amine |
| AM-1.70 | 2-(6-Methoxypyridin-2-yl)ethan-1-amine |
| AM-1.71 | 4-(2-Aminoethyl)-2-methoxyaniline |
| AM-1.72 | 3-Phenylpropan-1-amine |
| AM-1.73 | 2-(2,3-Dihydrobenzofuran-5-yl)ethan-1-amine•HCl |
| AM-1.74 | 2-(2-Methoxypyridin-4-yl)ethan-1-amine•HCl |
| AM-1.75 | (2,3-Dihydrobenzofuran-2-yl)methanamine |
| AM-1.76 | 3-(2-Aminoethyl)benzonitrile |
| AM-1.77 | 2-(3-(Trifluoromethyl)phenyl)ethan-1-amine |
| AM-1.78 | 2-(3-(Methylsulfonyl)phenyl)ethan-1-amine |
| AM-1.79 | 2-(4-Morpholinophenyl)ethan-1-amine |
| AM-1.80 | (1-(3,4-Dimethoxyphenyl)cyclopropyl)methanamine•HCl |
| AM-1.81 | 2-(4-Bromo-1H-pyrazol-1-yl)ethanamine•HCl |
| AM-1.82 | 2,2-Difluoro-2-(3-methoxyphenyl)ethan-1-amine |
| AM-1.83 | 2-(5-Isopropyl-1,2,4-oxadiazol-3-yl)ethan-1-amine•HCl |
| AM-1.84 | 2-(3-(Trifluoromethyl)isoxazol-5-yl)ethan-1-amine |
| AM-1.85 | 2-(3-(Trifluoromethyl)-1,2,4-oxadiazol-5-yl)ethan-1- |
| | amine•HCl |
| AM-1.86 | But-3-yn-1-amine•HCl |
| AM-1.87 | Benzyl 3-aminopropanoate 4-methylbenzenesulfonate |
| AM-1.88 | (S)-2-Amino-1-phenylethan-1-ol |
| AM-1.89 | 2-(3-Cyclopropyl-1,2,4-oxadiazol-5-yl)-2,2-difluoroethan- |
| | 1-amine |
| AM-1.90 | 2-Amino-1-(3-cyclopropyl-1,2,4-oxadiazol-5-yl)ethan-1-ol |
| AM-1.91 | 2-(2H-Benzo[d][1,2,3]triazol-2-yl)ethan-1-amine |
| AM-1.92 | 3-(Trifluoromethoxy)propan-1-amine |
| AM-1.93 | 2-([1,2,4]Triazolo[1,5-a]pyrimidin-2-yl)ethan-1-amine |
| AM-1-94 | 2-(1-(2-Methoxyethyl)-1H-pyrazol-4-yl)ethan-1-amine |
| AM-1.95 | 2-(6-Methoxypyridin-3-yl)ethan-1-amine |

Non-commercial amines are synthesised as described below.

### 2-Chroman-6-yl-ethylamine.HCl (AM-2.1)

Step 1: Potassium tert-butyl N-[2-(trifluoroboranuidyl)ethyl]carbamate (693 mg, 2.76 mmol) is added to a RT soln. of 6-bromochroman (600 mg, 2.73 mmol) and Cs₂CO₃ (2.67 g, 8.19 mmol) in PhMe (9.2 mL) and water (3 mL). After degassing the RM by bubbling argon through the soln., RuPhos 95% (134 mg, 0.273 mmol) and Pd(OAc)₂ (30.7 mg, 0.137 mmol) are added and the resulting mix. is stirred at 9500 for 18 h. The mix. is cooled to RT, then water and EtOAc are added and the RM is filtered over celite. The filtrate is extracted with EtOAc (3×), washed with brine, dried (MgSO₄), filtered, and concentrated. Purification by FC (eluting with 5% to 25% EtOAc in hept) gives (2-chroman-6-yl-ethyl)-carbamic acid tert-butyl ester (543 mg, 72%) as a pale yellow solid. LC-MS B: t_{R}=0.97 min; [M+H]⁺=222.04.

Step 2: 4 M HCl in dioxane (4 ml) is added to a RT soln. of (2-chroman-6-yl-ethyl)-carbamic acid tert-butyl ester (540 mg, 1.95 mmol) in dioxane (0.5 mL). The RM is stirred at RT for 4 h, then the mix. is concentrated in vacuo to give AM-2.1 (412 mg, 99%) as a white solid. LC-MS B: t_{R}=0.51 min; [M+H]⁺=219.41.

Listed in Table AM-2 below are amines that are prepared from the corresponding starting materials in analogy to the 2-step sequence described for AM-2.1.

TABLE AM-2

| | | | t_{R} [min] | MS-data m/z |
|---|---|---|---|---|
| Amine | SM | Name | LC-MS Method | [M + H]⁺ |
| AM-2.2 | 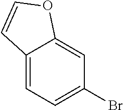 | 2-Benzofuran-6-yl-ethylamine•HCl | 0.5 B | 203.35 [+MeCN] |
| AM-2.3 | 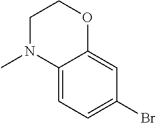 | 2-(4-Methyl-3,4-dihydro-2H- benzo[1,4]oxazin-7-yl)-ethylamine | 0.45 B | 193.40 |
| AM-2.4 | 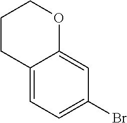 | 2-Chroman-7-yl-ethylamine•HCl | 0.51 B | 178.43 |
| AM-2.5 | 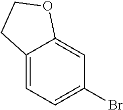 | 2-(2,3-Dihydro-benzofuran-6-yl)- ethylamine•HCl | 0.46 B | 164.12 |
| AM-2.6 | 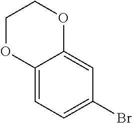 | 2-(2,3-Dihydro-benzo[1,4]dioxin- 6-yl)-ethylamine•HCl | 0.46 B | 180.26 |
| AM-2.7 | 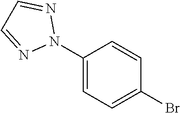 | 2-(4-[1,2,3]Triazol-2-yl-phenyl)- ethylamine•HCl | 0.49 B | 189.39 |
| AM-2.8 | 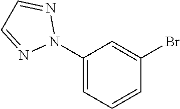 | 2-(3-[1,2,3]Triazol-2-yl-phenyl)- ethylamine•HCl | 0.50 B | 189.38 |
| AM-2.9 | 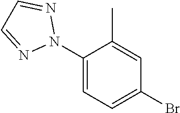 | 2-(3-Methyl-4-[1,2,3]triazol-2-yl- phenyl)-ethylamine•HCl | 0.76 I | 203.32 |
| AM-2.10 | 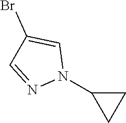 | 2-(1-Cyclopropyl-1H-pyrazol-4- yl)-ethylamine•HCl | 0.50 I | 152.26 |
| AM-2.11 | 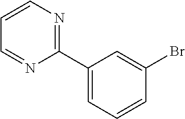 | 2-(3-Pyrimidin-2-yl-phenyl)- ethylamine•HCl | 0.48 B | 200.36 |
| AM-2.12 | 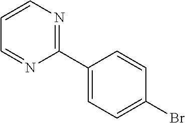 | 2-(4-Pyrimidin-2-yl-phenyl)- ethylamine•HCl | 0.47 B | 200.37 |
| AM-2.13 | 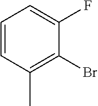 | 2-(2-Fluoro-6-methyl-phenyl)- ethylamine•HCl | 0.76 I | 154.24 |
| AM-2.14 | 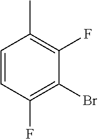 | 2-(2,6-Difluoro-3-methyl-phenyl)- ethylamine•HCl | 0.80 I | 172.14 |
| AM-2.15 | 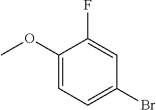 | 2-(3-Fluoro-4-methoxy-phenyl)- ethylamine•HCl | 0.67 I | 170.18 |
| AM-2.16 | 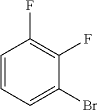 | 2-(2,3-Difluoro-phenyl)- ethylamine•HCl | 0.70 I | 158.21 |
| AM-2.17 | 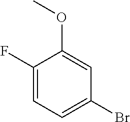 | 2-(4-Fluoro-3-methoxy-phenyl)- ethylamine•HCl | 0.73 I | 170.17 |
| AM-2.18 | 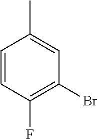 | 2-(2-Fluoro-5-methyl-phenyl)- ethylamine•HCl | 0.78 I | 154.17 |
| AM-2.19 | 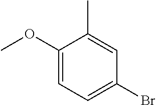 | 2-(4-Methoxy-3-methyl-phenyl)- ethylamine•HCl | 0.81 I | 166.12 |
| AM-2.20 | 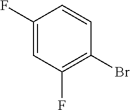 | 2-(2,4-Difluoro-phenyl)- ethylamine•HCl | 0.71 I | 158.22 |
| AM-2.21 | 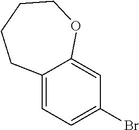 | 2-(2,3,4,5- Tetrahydrobenzo[b]oxepin-8- yl)ethan-1-amine•HCl | 0.55 B | 192.43 |

### 2-(3-(1,1-Difluoroethyl)phenyl)ethan-1-amine hydrochloride (AM-2.22)

Step 1: A mix. of 1-bromo-3-(1,1-difluoroethyl)benzene (200 mg, 0.91 mmol) and potassium (2-((tert-butoxycarbonyl)amino)ethyl)trifluoroborate (273 mg, 1.09 mmol) in PhMe (7 mL) and H₂O (2 mL) is degassed with Ar for 10 min before Cs₂CO₃ (884 mg, 2.71 mmol) and Pd(dppf)Cl₂.DCM (74 mg, 0.09 mmol) are added. The RM is degassed with Ar for a further 2 min and then heated to 100° C. for 2 h. After cooling to RT the RM is partitioned between sat. aq. NH₄Cl and DCM and extracted. The layers are separated, and the aq. phase is re-extracted with DCM (2×). The combined org. extracts are washed with brine, dried over Na₂SO₄, filtered and evaporated in vacuo. The crude product is purified by FC (eluting with 20% to 100% EtOAc in hept) to give tert-butyl (3-(1,1-difluoroethyl)phenethyl)carbamate as a white solid. LC-MS J: t_{R}=2.17 min; [M+H]⁺=208.1.

Step 2: The title compound is prepared from tert-butyl (3-(1,1-difluoroethyl)phenethyl)carbamate in analogy to the procedure described for AM-2.1 step 2. LC-MS J: t_{R}=1.77 min; [M+H]⁺=186.1.

### 2-(2-Cyclopropyl-2H-1,2,3-triazol-4-yl)ethan-1-amine hydrochloride (AM-2.23)

Step 1: Cu(OAc)₂ (1.23 g, 6.61 mmol) is added to a mix. of 4,5-dibromo-2H-1,2,3-triazole (1.5 g, 6.61 mmol), cyclopropyl boronic acid (1.17 g, 13.2 mmol), Na₂CO₃ (1.4 g, 13.2 mmol), and 2,2′-bipyridine (1.04 g, 6.61 mmol) in DCE (15 mL) and 2-methylfuran (15 mL) and the RM is heated to 80° C. for 48 h. The RM is filtered and the filter cake is rinsed with EtOAc. The filtrate is washed with brine, dried over Na₂SO₄, filtered and evaporated in vacuo. The crude product is purified by FC (eluting with 0% to 30% EtOAc in hept) to give 4,5-dibromo-2-cyclopropyl-2H-1,2,3-triazole as a yellow solid. LC-MS D: t_{R}=0.92 min; No ionisation.

Step 2: tert-Butyl (2-(5-bromo-2-cyclopropyl-2H-1,2,3-triazol-4-yl)ethyl)carbamate is prepared from 4,5-dibromo-2-cyclopropyl-2H-1,2,3-triazole following the procedure described for AM-2.1 step 1. LC-MS B: t_{R}=0.91 min; [M+H]⁺=331.06.

Step 3: A soln. of tert-butyl (2-(5-bromo-2-cyclopropyl-2H-1,2,3-triazol-4-yl)ethyl)carbamate (110 mg, 0.33 mmol) in EtOH (3 mL) is evacuated/purged with N₂ (3×) before 10% Pd/C (23 mg, 5 mol %) is added. The RM is evacuated/purged with H₂ (3×) and stirred under a H₂ atm for 16 h. The RM is filtered through a pad of celite and the filtrate concentrated in vacuo to give tert-butyl (2-(2-cyclopropyl-2H-1,2,3-triazol-4-yl)ethyl)carbamate as a white solid. LC-MS I: t_{R}=0.83 min; [M+H]⁺=253.28.

Step 4: The title compound is prepared from tert-butyl (2-(2-cyclopropyl-2H-1,2,3-triazol-4-yl)ethyl)carbamate in analogy to the procedure described for AM-2.1 step 2. LC-MS I: t_{R}=0.46 min; [M+H]⁺=153.24.

### 2-(3,4-Dihydro-2H-benzo[b][1,4]dioxepin-7-yl)-ethylamine.HCl (AM-3.1)

Step 1: Di-tert-butyl dicarbonate (2.33 g, 10.5 mmol) is added to a RT suspension of dopamine hydrochloride (2.0 g, 10.5 mmol) and NaHCO₃ (886 mg, 10.5 mmol) in THF (50 mL) and the mix. is stirred at RT for 2 h. The product is extracted with EtOAc (3×) and the combined org. layers are dried (MgSO₄), filtered, and concentrated. Purification by FC (eluting with 5% MeOH in DCM) yields tert-butyl (3,4-dihydroxyphenethyl)carbamate (2.34 g, 88%) as a white solid. LC-MS I: t_{R}=0.64 min; [M−H]⁻=252.00.

Step 2: 1,3-Dibromopropane (1.01 mL, 9.75 mmol) is added to a RT suspension of tert-butyl (3,4-dihydroxyphenethyl)carbamate (2.24 g, 8.86 mmol) and K₂CO₃ (3.13 g, 22.2 mmol) in DMF (10 mL) and the resulting mix. is stirred at RT overnight. The RM is directly purified by prep. HPLC (basic) to give tert-butyl (2-(3,4-dihydro-2H-benzo[b][1,4]dioxepin-7-yl)ethyl)carbamate (1.25 g, 48%) as a slightly brownish oil. LC-MS I: t_{R}=0.98 min; [M+H]⁺=294.05.

Step 3: 4 M HCl in dioxane (5.4 mL) is added to a RT soln. of tert-butyl (2-(3,4-dihydro-2H-benzo[b][1,4]dioxepin-7-yl)ethyl)carbamate (1.25 g, 4.28 mmol) in dioxane (20 mL). The RM is stirred at RT for 18 h, then the mix. is concentrated in vacuo to give AM-3.1 (931 mg, 100%) as a white solid. LC-MS I: t_{R}=0.68 min; [M+H]⁺=194.13.

### 2-(3-(Methoxy-d3)phenyl)ethan-1-amine.HCl (AM-3.2)

Step 1: CD₃I (0.25 mL, 4.0 mmol) is added to a RT mix. of tert-butyl (3-hydroxyphenethyl)carbamate (638 mg, 2.7 mmol) and K₂CO₃ (557 mg, 4.0 mmol) in DMF (5 mL) and the RM is heated to 50° C. for 36 h. The RM is cooled to RT and partitioned between H₂O and EtOAc and extracted. The layers are separated and the aq. phase is re-extracted with EtOAc (2×). The combined org. layers are washed with brine, dried over Na₂SO₄, filtered and evaporated in vacuo to give the crude product that is purified by FC (eluting with 0% to 100% EtOAc in hept) to give tert-butyl (3-(methoxy-d3)phenethyl)carbamate as a colourless oil. LC-MS J: t_{R}=2.09 min; [M+H-Me]⁺=240.1.

Step 2: The title compound is prepared from tert-butyl (3-(methoxy-d3)phenethyl)carbamate in analogy to the procedure described for AM-2.1 step 2. LC-MS J: t_{R}=1.47 min; [M+H]⁺=155.1.

### 2-(2-Fluoro-4-methoxy-phenyl)-ethylamine.HCl (AM-4.1)

Step 1: Molecular sieves 4A (100 mg) are added to a RT soln. of 2-fluoro-4-methoxybenzaldehyde (1.25 g, 7.95 mmol) in nitromethane (16 mL), then butylamine (0.1 mL, 0.938 mmol) and acetic acid (0.01 mL, 1.62 mmol) are added and the mix. is heated to 90° C. for 1 h. The RM is concentrated and the residue is partitioned between EtOAc and water. The org. layer is washed with water and brine and concentrated in vacuo. Purification by FC (eluting with 0% to 20% EtOAc in hept) yields 2-fluoro-4-methoxy-1-2-nitro-vinyl-benzene (1.06 g, 68%) as a yellowish solid. LC-MS B: t_{R}=0.92 min; No ionisation.

Step 2: Boron trifluoride diethyl etherate (4.19 mL, 32.3 mmol) is added to a 0° C. soln. of NaBH₄ (996 mg, 25.8 mmol) in THF (40 mL). The mix. is stirred at 0° C. for 10 min, then at RT for 15 min before a soln. of 2-fluoro-4-methoxy-1-2-nitro-vinyl-benzene (1.06 g, 5.38 mmol) in THF (10 mL) is added dropwise and the mix. refluxed at 70° C. for 3 h, then allowed to reach RT overnight. The RM is cooled to 0° C. before 2 N HCl (35 mL, 69.9 mmol) is added dropwise. After addition, the mix. is stirred at 0° C. for 10 min, then at RT for 15 min, before the mix. is heated to 80° C. for 1 h. The RM is cooled to RT and the org. solvent is evaporated, and the remaining aq. layer is cooled to 0° C., before being basified with 10% aq. NaOH. The product is extracted with EtOAc (3×), and the combined org. layers are washed with brine, dried (MgSO₄), filtered, and concentrated. The residue, well dried under HV, is dissolved in DCM (10 mL) and cooled to 0° C. before 4 M HCl in dioxane (1.61 mL, 6.45 mmol) is added and the resulting mix. stirred for 1 h. The RM is concentrated and triturated with Et₂O (2×) to yield the title compound AM-4.1 (957 mg, 87%) as a beige powder. LC-MS B: t_{R}=0.49 min; [M+H]⁺=170.08.

### 2-(4-Bromo-2,6-difluoro-phenyl)-ethylamine (AM-4.2)

The title compound is prepared from 4-bromo-2,6-difluorobenzaldehyde in analogy to the synthesis described for AM-4.1. LC-MS B: t_{R}=0.53 min; [M+H]⁺=235.98.

### 2-(2,6-Difluoro-4-methoxy-phenyl)-ethylamine (AM-4.3)

Step 1: Ammonium acetate (179 mg, 2.28 mmol) is added to a RT soln. of 2,6-difluoro-4-methoxybenzaldehyde (1.0 g, 5.69 mmol) in nitromethane (7 mL) and the resulting mix. is refluxed for 40 min. The RM is evaporated and the residue partitioned between water and DCM. The aq. layer is extracted with DCM (2×) and the combined org. layers are washed with brine, dried (MgSO₄), filtered, and concentrated to obtain 1,3-difluoro-5-methoxy-2-(2-nitro-vinyl)-benzene (1.25 g) as an orange oil which is used as such in the next step. LC-MS B: t_{R}=0.96 min; [M+H]⁺=216.12.

Step 2: The title compound is prepared from 1,3-difluoro-5-methoxy-2-(2-nitro-vinyl)-benzene in analogy to the procedure described for AM-4.1 step 2. LC-MS B: t_{R}=0.51 min; [M+H]⁺=188.32.

### 2-(3,4-Difluoro-phenyl)-ethylamine.HCl AM-4.4

Step 1: 1,2-Difluoro-4-(2-nitro-vinyl)-benzene is prepared in analogy to the procedure described for AM-4.1, step 1. LC-MS C: t_{R}=0.75 min; No ionisation.

Step 2: Concentrated H₂SO₄ (0.710 mL) is added to a 0° C. suspension of LiAlH₄ (1.06 g, 26.65 mmol) in THF (35 mL).

After stirring for 20 min, a soln. of 1,2-difluoro-4-(2-nitro-vinyl)-benzene (1.10 g, 5.97 mmol) in THF (5 mL) is added dropwise and stirring is continued for 10 min before the cooling bath is removed and the RM is slowly heated to a gentle reflux. After 5 min, the mix. is cooled to 0° C. and carefully hydrolised by dropwise addition of ⁱPrOH (4.4 mL), followed by 2 M aq. NaOH soln. (3.1 mL). The resulting suspension is filtered, and the filter cake rinsed with THF. The filtrate is concentrated and the free amine is dissolved in Et₂O (20 mL) containing ⁱPrOH (0.72 mL) and acidified with 2 M HCl in Et₂O (11.4 mL). The resulting suspension is filtered and the filter cake washed with Et₂O to give the title compound (440 mg, 38%) as a white solid that is further dried under HV. LC-MS C: t_{R}=0.40 min; [M+H]⁺=199.3.

### 2-(4,5-dimethylisoxazol-3-yl)ethan-1-amine (AM-5.1)

Step 1: In a Dean Stark Apparatus, pTsOH monohydrate (11.1 mg, 0.06 mmol) is added to a RT soln. of ethyl acetoacetate (1.46 mL, 11.4 mmol) and pyrrolidine (1.92 mL, 22.8 mmol) in PhMe (50 mL) and the resulting mix. is refluxed for 2 h. The volatiles are removed and 3-pyrrolidin-1-yl-but-2-enoic acid ethyl ester (2.03 g, 97%) as an orange oil is used as such in the next step. LC-MS B: t_{R}=0.39 min; [M+H]⁺=184.45.

Step 2: A soln. of SO₃ Pyridine complex (11.0 g, 69.2 mmol) in DMSO (39.3 mL) is added dropwise to 0° C. soln. of 3-(Boc-amino)-1-propanol (4.88 mL, 27.7 mmol) and DIPEA (14.2 mL, 083 mmol) in DCM (83.1 mL). The RM is stirred at 0° C. for 1 h, then at RT for 1 h. The mix. is diluted with HCl and water, then extracted with DCM (3×). The combined org. extracts are washed with water and brine, dried (Na₂SO₄), filtered, and concentrated to give (3-oxo-propyl)-carbamic acid tert-butyl ester (4.81 g, 100%) as a colourless oil which is used as such in the next step.

Step 3: A soln. of hydroxylamine hydrochloride (3.90 g, 0.06 mol) in H₂O (25 mL) and a soln. of sodium acetate (9.20 g, 0.11 mol) in H₂O (25 mL) are added to a vigorously stirred soln. of (3-oxo-propyl)-carbamic acid tert-butyl ester (4.81 g, 0.028 mol) in EtOH (100 mL). The resulting suspension is stirred at RT for 18 h and then another 3 h at 50° C. The volatiles are removed, and the residue is partitioned between EtOAc and water. The layers are separated and the aq. layer is further extracted with EtOAc. The combined org. extracts are washed with brine, dried (Na₂SO₄), filtered, and concentrated. Purification by FC (eluting with 50% EtOAc in hept) gives (3-hydroxyimino-propyl)-carbamic acid tert-butyl ester (4.5 g, 86%) as a colourless oil. LC-MS B: t_{R}=0.58 min; [M+H]⁺=189.43.

Step 4: NCS (2.23 g, 16.4 mmol) is added to a RT soln. of (3-hydroxyimino-propyl)-carbamic acid tert-butyl ester (2.80 g, 14.9 mmol) in DCM (80 mL) and the resulting mix. is stirred at RT for 2 h. The mix. is evaporated and directly purified by FC (eluting with 50% EtOAc in hept) to give tert-butyl (3-chloro-3-(hydroxyimino)propyl)carbamate (2.02 g, 61%) as an orange oil. LC-MS B: t_{R}=0.72 min; [M(³⁵Cl)+H]⁺=223.37.

Step 5: A soln. of 3-pyrrolidin-1-yl-but-2-enoic acid ethyl ester (1.83 g, 10 mmol) in DCM (15 mL) followed by TEA (2.56 mL, 0.0181 mol) is added to a RT soln. of tert-butyl (3-chloro-3-(hydroxyimino)propyl)carbamate (2.02 g, 9.07 mmol) in DCM (15 mL) and the resulting mix. is stirred for 15 min. The RM is concentrated and the residue directly purified by FC (eluting with 50% EtOAc in hept) to yield 3-(2-tert-butoxycarbonylamino-ethyl)-5-methyl-isoxazole-4-carboxylic acid ethyl ester (1.60 g) still containing starting material, therefore the product is dissolved in DCM and washed with 2 M aq. HCl. The org. layer is washed with brine, dried (Na₂SO₄), filtered, and concentrated to give 3-(2-tert-butoxycarbonylamino-ethyl)-5-methyl-isoxazole-4-carboxylic acid ethyl ester (1.25 g, 46%) as slightly yellow oil. LC-MS B: t_{R}=0.93 min; [M+H]⁺=299.30.

Step 6: LAH (76 mg, 2.01 mmol) in Et₂O (15 mL) is added dropwise to a 0° C. soln. of 3-(2-tert-butoxycarbonylamino-ethyl)-5-methyl-isoxazole-4-carboxylic acid ethyl ester (500 mg, 1.68 mmol) in Et₂O (5 mL). After addition, the resulting mix. is warmed to RT and stirred for 1.5 h. The mix. is cooled to 0° C. and very carefully quenched with EtOAc followed by addition of a saturated aq. Rochelle's salt soln. The resulting mix. is warmed to RT and then vigorously stirred for 30 min after which two layers are formed. The layers are separated and the aq. layer is re-extracted with EtOAc (2×). The combined org. extracts are washed with brine, dried (Na₂SO₄), filtered, and evaporated. Purification by FC (eluting with 50% EtOAc in hept) followed by prep. HPLC (basic) gives [2-(4-hydroxymethyl-5-methyl-isoxazol-3-yl)-ethyl]-carbamic acid tert-butyl ester (135 mg, 31%) as a white solid. LC-MS I: t_{R}=0.67 min; [M+H]⁺=257.20.

Step 7: 4 M HCl in dioxane (1 mL, 3.99 mmol) is added to a RT soln. of [2-(4-hydroxymethyl-5-methyl-isoxazol-3-yl)-ethyl]-carbamic acid tert-butyl ester (100 mg, 0.39 mmol) in dioxane (2 mL) and the resulting mix. is stirred at RT for 6 d. The mix. is concentrated to yield 2-(4-chloromethyl-5-methyl-isoxazol-3-yl)-ethylamine (HCl salt) (77 mg, 93%) as a colourless oil which is used as such in the next step. LC-MS I: t_{R}=0.56 min; No ionisation.

Step 8: Pd/C (10 mg, 0.01 mmol) is added to a RT soln. (degassed) of 2-(4-chloromethyl-5-methyl-isoxazol-3-yl)-ethylamine (HCl salt) (19 mg, 0.09 mmol) in EtOH (0.5 mL) and EtOAc (0.5 mL). The RM is stirred at RT for 30 min under a H₂ atm. The mix. is filtered, and the filtrate evaporated to yield AM-5.1 (45 mg, 89%) as a yellow solid which is used as such in the next step. LC-MS I: t_{R}=0.51 min; [M+H]⁺=141.20.

### 2-(3-Cyclopropylisoxazol-5-yl)ethan-1-amine.HCl (AM-5.2)

Step 1: A soln. of DIAD (61.7 mL, 318 mmol) in THF (350 mL) is added dropwise to a 0° C. soln. of but-3-yn-1-ol (22.9 mL, 318 mmol), isoindoline-1,3-dione (44.5 g, 302 mmol) and PPh₃ (83 g, 318 mmol) in THF (1500 mL) and the RM is stirred for 1 h. The RM is concentrated in vacuo and the residue is dissolved in hot PhMe (370 mL) before MeOH (210 mL) is slowly added. The RM is cooled to RT and MeOH is added until a white solid precipitates. The RM is partially concentrated before the solid is collected by filtration washing with cold PhMe and then air dried to give 2-(but-3-yn-1-yl)isoindoline-1,3-dione. LC-MS F: t_{R}=1.81 min; No ionisation.

Step 2: Na₂CO₃ (22.7 g, 214 mmol) is carefully added to a RT soln. of hydroxylamine.HCl (37.2 g, 535 mmol) in H₂O (125 mL) followed by the slow addition of a soln. of cyclopropanecarbaldehyde (26.7 mL, 357 mmol) in EtOH (100 mL). The RM is stirred for 1 h and then partitioned between H₂O and EtOAc and extracted. The layers are separated and the aq. phase is re-extracted with EtOAc (2×). The combined org. layers are washed with brine, dried over Na₂SO₄, filtered and evaporated in vacuo to give the crude product that is recrystallised from n-hept to give E/Z-cyclopropanecarbaldehyde oxime as a white solid. LC-MS F: t_{R}=2.13 & 2.30 min; No ionisation.

Step 3: NCS (34.3 g, 257 mmol) is added portion wise to a 0° C. soln. of cyclopropanecarbaldehyde oxime (19.3 g, 226 mmol) and pyridine (0.83 mL, 10.3 mmol) in DMF (100 mL) and the RM is stirred for 3 h. A soln. of 2-(but-3-yn-1-yl)isoindoline-1,3-dione (21.0 g, 103 mmol) in DMF (100 mL) followed by TEA (28.7 mL, 206 mmol) are added and the RM is stirred for 3 h. The RM is partitioned between H₂O and DCM and extracted. The layers are separated and the aq. phase is re-extracted with DCM (2×). The combined org. layers are washed with brine, dried over Na₂SO₄, filtered and evaporated in vacuo to give the crude product that is triturated with MeOH to give 2-(2-(3-cyclopropylisoxazol-5-yl)ethyl)isoindoline-1,3-dione as a white solid. LC-MS J: t_{R}=1.91 min; [M+H]⁺=283.1.

Step 4: Hydrazine.H₂O (9.44 mL, 194 mmol) is added to a RT suspension of 2-(2-(3-cyclopropylisoxazol-5-yl)ethyl)isoindoline-1,3-dione (28.0 g, 97 mmol) in EtOH (100 mL) and the RM is heated to 80° C. for 5 h. The RM is cooled to RT and filtered washing with EtOH. The filtrate is concentrated in vacuo and the residue is suspended in Et₂O and re-filtered washing with Et₂O. The filtrate is partially concentrated before 1M HCl in Et₂O (100 mL) is added and the precipitate is filtered and dried in vacuo to give the title compound as a white solid. LC-MS J: t_{R}=1.42 min; [M+H]⁺=153.1.

### 2-(3-(Difluoromethyl)isoxazol-5-yl)ethan-1-amine (AM-5.3)

Step 1: Imidazole (1.94 g, 28.5 mmol) is added to a RT soln. of but-3-yn-1-ol (1.0 mL, 14.3 mmol) in THF (25 mL) followed by TBDMSCl (2.58 g, 17.1 mmol) and the RM is stirred for 16 h. ⁱPr₂O (25 mL) is added and the precipitate is filtered and washed with additional iPr₂O. The filtrate is washed with sat. aq. NaHCO₃, brine, dried over Na₂SO₄, filtered and evaporated in vacuo to give (but-3-yn-1-yloxy)(tert-butyl)dimethylsilane as a colourless oil. ¹H NMR (CDCl₃) δ: 3.76 (t, J=7.1 Hz, 2H), 2.43 (td, J=7.1, 2.7 Hz, 2H), 1.98 (t, J=2.7 Hz, 1H), 0.92 (s, 9H), 0.10 (s, 6H).

Step 2: nBuLi (1.6 M in hex, 7.9 mL, 12.6 mmol) is added to a −78° C. soln. of (but-3-yn-1-yloxy)(tert-butyl)dimethylsilane (1.85 g, 9.0 mmol) in THF (15 mL) and the RM is warmed to −15° C. and stirred for 15 min before being cooled back to −40° C. Ethyl difluoroacetate (1.24 mL, 11.7 mmol) is then added dropwise followed by boron trifluoride etherate (1.55 mL, 12.2 mmol) and the RM is warmed to RT and stirred for 16 h. The reaction is quenched with cold sat. aq. NH₄Cl soln. and extracted with EtOAc (3×). The combined org. extracts are washed with brine, dried over Na₂SO₄, filtered and evaporated in vacuo. The crude product is purified by FC (eluting with 20% to 50% DCM in hept) to give 6-((tert-butyldimethylsilyl)oxy)-1,1-difluorohex-3-yn-2-one as a yellow oil. ¹H NMR (CDCl₃) δ: 5.73 (t, J=54.2 Hz, 1H), 3.82 (t, J=6.5 Hz, 2H), 2.68 (t, J=6.6 Hz, 2H), 0.89 (s, 9H), 0.08 (s, 6H).

Step 3: Hydroxylamine hydrochloride (0.71 g, 10.2 mmol) followed by CuO (121 mg, 0.85 mmol) are added to a RT soln. of 6-((tert-butyldimethylsilyl)oxy)-1,1-difluorohex-3-yn-2-one (2.22 g, 8.5 mmol) in THF (20 mL) and the RM is stirred for 16 h. NaHCO₃ (0.85 g, 10.2 mmol) is added and the RM is stirred for 1 h before the RM is filtered over a short pad of silica gel (eluting with 1:1 Et₂O:THF) to give a mix. of 5-(2-((tert-butyldimethylsilyl)oxy)ethyl)-3-(difluoromethyl)isoxazole and 2-(3-(difluoromethyl)isoxazol-5-yl)ethan-1-ol.

Step 4: TBAF (1.0 M in THF, 6.0 mL, 6.0 mmol) is added to a RT soln. of 5-(2-((tert-butyldimethylsilyl)oxy)ethyl)-3-(difluoromethyl)isoxazole and 2-(3-(difluoromethyl)isoxazol-5-yl)ethan-1-ol (2.35 g, 8.5 mmol—estimated) in THF (50 mL) and Et₂O (50 mL) and the RM is stirred for 16 h. The RM is partitioned between sat. aq. NH₄Cl and EtOAc and extracted. The layers are separated, and the aq. phase is re-extracted with EtOAc (2×). The combined org. extracts are washed with brine, dried over Na₂SO₄, filtered and evaporated in vacuo. The crude product is purified by FC (eluting with 0% to 6% MeOH in DCM) to give 2-(3-(difluoromethyl)isoxazol-5-yl)ethan-1-ol as a yellow oil. ¹H NMR (DMSO) δ: 7.23 (t, J=53.2 Hz, 1H), 6.64 (s, 1H), 4.93 (t, J=5.3 Hz, 1H), 3.72 (q, J=6.0 Hz, 2H), 2.96 (t, J=6.3 Hz, 2H).

Step 5: A soln. of DIAD (0.59 mL, 3.0 mmol) in THF (2 mL) is added dropwise to a 0° C. soln. of 2-(3-(difluoromethyl)isoxazol-5-yl)ethan-1-ol (412 mg, 2.5 mmol), isoindoline-1,3-dione (409 mg, 2.8 mmol) and PPh₃ (7.95 g, 3.0 mmol) in THF (20 mL) and the RM is stirred for 16 h. The RM is concentrated in vacuo and the residue is purified by FC (eluting with 0% to 30% EtOAc in hept) to give 2-(2-(3-(difluoromethyl)isoxazol-5-yl)ethyl)isoindoline-1,3-dione. LC-MS J: t_{R}=1.98 min; [M+H]⁺=293.1.

Step 6: Hydrazine.H₂O (380 μL, 0.77 mmol) is added to a RT suspension of 2-(2-(3-(difluoromethyl)isoxazol-5-yl)ethyl)isoindoline-1,3-dione (113 mg, 0.39 mmol) in EtOH (4 mL) and the RM is heated to 80° C. for 2 h. The RM is cooled to RT and filtered washing with EtOH. The filtrate is concentrated in vacuo and the residue is suspended in Et₂O and re-filtered washing with Et₂O. The filtrate is concentrated in vacuo to give the title compound as a white solid. LC-MS I: t_{R}=0.53 min; [M+H+MeCN]⁺=204.32.

### 2-(2,4,6-Trifluoro-phenyl)-ethylamine.HCl (AM-6.1)

Borane tetrahydrofuran complex 1M soln. In THF (15.5 mL, 15.5 mmol) is added dropwise to 0° C. soln. of 2,4,6-trifluorophenylacetonitrile (1.00 g, 5.73 mmol) in THF (10 mL) and the RM is stirred at RT overnight. The RM is cooled to 0° C. before MeOH (5 mL) is added dropwise, then the soln. is stirred at RT for 1 h before being concentrated. The residue is cooled to 0° C., then 1.25 M HCl in MeOH (15.0 mL) is added dropwise and the resulting suspension is stirred a RT for 2 days, then the solvent is removed, and the residue is triturated in Et₂O. The product is isolated by filtration and washed with Et₂O to yield AM-6.1 (833 mg, 69%) as a white solid. LC-MS B: t_{R}=0.45 min; [M+H]⁺=176.26.

### 2-[3-(3-Methoxy-phenyl)-[1,2,4]oxadiazol-5-yl]-ethylamine (AM-7.1)

Step 1: NaHCO₃ (1.55 g, 18.4 mmol), followed by hydroxylamine hydrochloride (1.29 g, 18.4 mmol) are added to a RT soln. of 3-methoxybenzonitrile (1.0 g, 7.36 mmol) in MeOH (15 mL) and the resulting white suspension is refluxed (70° C.) overnight. The mix. is concentrated and the residue is diluted with EtOAc and washed with brine, dried (MgSO₄), filtered, and concentrated to yield N-hydroxy-3-methoxy-benzamidine (1.67 g, 137%) as a yellow oil which is used as such in the next step. LC-MS B: t_{R}=0.38 min; [M+H]⁺=167.11.

Step 2: TBTU (3.49 g, 10.9 mmol) is added to a 0° C. soln. of Boc-beta-ala-OH (1.73 g, 9.06 mmol), N-hydroxy-3-methoxy-benzamidine (1.67 g, 9.06 mmol) and DIPEA (4.65 mL, 27.2 mmol) in DCM (45 mL). The ice bath is removed and the mix. is stirred at RT for 18 h. The RM is concentrated and the residue is partitioned between EtOAc (50 mL) and water (50 mL) and the resulting solid is filtered off to yield the intermediate (2-{[[hydroxyimino]-(3-methoxy-phenyl)-methyl]-carbamoyl}-ethyl)-carbamic acid tert-butyl ester (1.856 g, 61%). To this white solid is added dioxane (50 mL) and the RM is refluxed (90° C.) for 24 h. The mix. is concentrated to yield {2-[3-(3-methoxy-phenyl)-[1,2,4]oxadiazol-5-yl]-ethyl}-carbamic acid tert-butyl ester (1.90 g, 109%) as a colourless oil which is used as such in the next step. LC-MS B: t_{R}=0.95 min; [M+H]⁺=320.12.

Step 3: TFA (4.59 mL, 60 mmol) is added to a RT soln. of {2-[3-(3-methoxy-phenyl)-[1,2,4]oxadiazol-5-yl]-ethyl}-carbamic acid tert-butyl ester (1.915 g, 6.0 mmol) in DCM (40 mL) and the mix. is stirred at RT for 1 d. The mix. is neutralised with a saturated aq. soln. of NaHCO₃ (50 mL), then DCM (50 mL) is added. The two layers are separated and the aq. layer is extracted with DCM (50 mL). The combined org. layers are dried (MgSO₄), filtered, and concentrated to yield AM-7.1 (1.14 g, 86%) as a yellow oil. LC-MS B: t_{R}=0.54 min; [M+H]⁺=220.22.

### 2-[3-(3,5-Dimethyl-phenyl)-[1,2,4]oxadiazol-5-yl]-ethylamine (AM-7.2)

The title compound is prepared from 3,5-dimethylbenzonitrile following the 3-step synthesis described for AM-7.1. LC-MS E: t_{R}=0.53 min; [M+H]⁺=218.22.

### 2-[3-(2-Trifluoromethoxy-phenyl)-[1,2,4]oxadiazol-5-yl]-ethylamine.HCl (AM-7.3)

Steps 1&2: The title compound is prepared from 3,2-trifluoromethoxy-benzonitrile following the synthesis described for AM-7.1, steps 1&2 to yield {2-[3-(2-trifluoromethoxy-phenyl)-[1,2,4]oxadiazol-5-yl]-ethyl}-carbamic acid tert-butyl ester. LC-MS E: t_{R}=0.88 min; [M+H]⁺=318.04.

Step 3: 4 M HCl in dioxane (6 mL) is added to a RT soln. of {2-[3-(2-trifluoromethoxy-phenyl)-[1,2,4]oxadiazol-5-yl]-ethyl}-carbamic acid tert-butyl ester (425 mg, 1.14 mmol) in dioxane (3 mL) and the mix. is stirred at RT for 18 h. The RM is concentrated to yield AM-7.3 (0.41 g, 119%) as a brownish oil. LC-MS E: t_{R}=0.53 min; [M+H]⁺=273.93.

### 2-[5-(2-Trifluoromethoxy-phenyl)-4H-[1,2,4]triazol-3-yl]-ethylamine (AM-8.1)

Step 1: K₂CO₃ (487 mg, 3.53 mmol) is added to a RT soln. of tert-butyl N-(2-cyanoethyl)carbamate (1.20 g, 7.05 mmol) and 2-(trifluoromethoxy)benzoic acid hydrazide (1.55 g, 7.05 mmol) in n-butanol (50 mL) and the resulting suspension is heated to 120° C. for 6.5 h, then stirred at RT overnight, and re-heated to 120° C. for another 4.5 h. The mix. is concentrated, and the residue is diluted with DCM and acidified with 1 N HCl. The two layers are separated and the aq. layer is extracted with DCM. The combined org. layers are concentrated and purified by FC (eluting with 10% to 30% EtOAc in hept) to yield {2-[5-(2-trifluoromethoxy-phenyl)-4H-[1,2,4]triazol-3-yl]-ethyl}-carbamic acid tert-butyl ester (678 mg, 26%) as a colourless oil. LC-MS A: t_{R}=0.80 min; [M+H]⁺=373.15.

Step 2: 4 M HCl in dioxane (30 mL, 30 mmol) is added dropwise to a 0° C. soln. of {2-[5-(2-trifluoromethoxy-phenyl)-4H-[1,2,4]triazol-3-yl]-ethyl}-carbamic acid tert-butyl ester (678 mg, 1.82 mmol) in DCM (20 mL). The resulting RM is stirred at RT for 2 h, then the RM is concentrated to yield AM-8.1 (515 mg, 104%) as a yellow oil, which is used as such in the next step. LC-MS A: t_{R}=0.52 min; [M+H]⁺=273.14.

### 2-(3-Methoxy-4-[1,2,3]triazol-2-yl-phenyl)-ethylamine.HCl (AM-9.1)

Step 1: 1H-1,2,3-Triazole (5.0 g, 0.072 mmol) is diluted with water (35 mL) and heated to 50° C. before Br₂ (23.1 g, 0.145 mmol) is added dropwise (exothermic). The oil bath is replaced with a water bath to keep the internal temperature below 50° C. After 15 min the resulting orange suspension is quenched with 2 M aq. NaOH (5 mL) and 40% sodium bisulfite soln. (2 mL). A 32% aq. NaOH soln. is then added until pH 7 followed by additional 40% sodium bisulfite soln. (10 mL). Due to the exotherm the suspension is cooled to RT before it is filtered. The cake is rinsed with water (3×10 mL) and the filtrate is concentrated to yield 4,5-dibromo-2H-[1,2,3]triazole (14.86 g, 90%) as a slightly yellowish solid.

Step 2: K₂CO₃ (3.96 g, 28.6 mmol) and 4,5-dibromo-2H-[1,2,3]triazole (6.50 g, 28.6 mmol) are added to a RT soln. of 2-fluoro-5-nitroanisole (5.00 g, 28.6 mmol) in DMF (40 mL) and the mix. is stirred at 45° C. for 3 d. The mix. is diluted with H₂O and the formed precipitate is filtered off, washed with water and the collected solid dried under HV to yield 4,5-dibromo-2-(2-methoxy-4-nitro-phenyl)-2H-[1,2,3]triazole (7.51 g, 69%) as a white solid. LC-MS B: t_{R}=1.03 min; No ionisation.

Step 3: Pd(OH)₂ (20%, 1.04 g, 1.96 mmol) is added to a RT soln. (degassed) of 4,5-dibromo-2-(2-methoxy-4-nitro-phenyl)-2H-[1,2,3]triazole (7.4 g, 0.020 mol) in MeOH (70 mL) and the resulting mix. is stirred at RT for 2 h under a H₂ atm. The mix. is filtered over celite which is then copiously washed with MeOH. The filtrate is concentrated and purified by FC (eluting first with EtOAc/hept, then with 100% EtOAc and eventually with 10% MeOH in DCM. The isolated product (130%) is repurified by prep. HPLC (basic) to yield 3-methoxy-4-[1,2,3]triazol-2-yl-phenylamine (2.87 g, 75%) as a brown oil. LC-MS I: t_{R}=0.53 min: [M+H]⁺=191.33.

Step 4: tert.-Butyl nitrite (0.314 mL, 2.65 mmol) is added slowly to a 60° C. soln. of CuBr₂ (537 mg, 2.41 mmol) in MeCN (3 mL). A soln. of 3-methoxy-4-[1,2,3]triazol-2-yl-phenylamine (500 mg, 2.41 mmol) in MeCN (3 mL) is added dropwise and after addition the mix. is allowed to reach RT. The mix. is quenched with a soln. of sulfamic acid (47 mg, 0.48 mmol) in water (1 mL) followed by the addition of 2 M aq. HCl (3 mL). MeCN is evaporated before EtOAc is added and the layers are separated. The org. layer is washed with 2 M aq. HCl, water and brine, then dried (Na₂SO₄), filtered, and concentrated. Purification by FC (eluting with 10% to 20% EtOAc in hex) gives 2-(4-bromo-2-methoxyphenyl)-2H-1,2,3-triazole (0.27 g, 46%) as a yellow oil. LC-MS I: t_{R}=0.87 min; [M+H]⁺=254.13.

Step 5&6: The title compound is prepared from 2-(4-bromo-2-methoxyphenyl)-2H-1,2,3-triazole following the 2-step sequence described for AM-2.1. LC-MS I: t_{R}=0.61 min; [M+H]⁺=260.26.

### 2-(4-Pyrazin-2-yl-phenyl)-ethylamine (AM-10.1)

Step 1: K₂CO₃ 2 M in water (2 mL) is added to a RT soln. of 4-(2-nitroethyl)phenylboronic acid (200 mg, 1.03 mmol) and 2-bromopyrazine (168 mg, 1.03 mmol) in dioxane (8 mL). The soln. is degassed for 2 min with argon, then Pd(PPh₃)₄ (35.6 mg, 0.0308 mmol) is added and the mix. heated to 80° C. for 18 h. To the mix. is added water and EtOAc. The layers are separated and the aq. layer is re-extracted with EtOAc. The combined org. layers are dried (MgSO₄), filtered, and concentrated. Purification by FC (EtOAc/hept 2:3) yields 2-[4-(2-nitro-ethyl)-phenyl]-pyrazine (88 mg, 37%) as a yellow oil. LC-MS B: t_{R}=0.84 min; [M+H]⁺=230.10.

Step 2: Pd/C (10%, 13.5 mg, 0.019 mmol) is added to a RT soln. of 2-[4-(2-nitro-ethyl)-phenyl]-pyrazine (88 mg, 0.384 mmol) in EtOH/THF and the mix. is stirred at RT for 18 h under a H₂ atm. The mix. is filtered and concentrated. Purification by prep. HPLC (basic) gives AM-10.1 (65 mg, 85%) as a yellow solid. LC-MS B: t_{R}=0.48 min; [M+H]⁺=200.20.

### 2-(3-Pyrazin-2-yl-phenyl)-ethylamine (AM-10.2)

The title compound is prepared from (3-(2-nitroethyl)phenyl)boronic acid following the 2 step procedure described for AM-10.1. LC-MS B: t_{R}=0.48 min; [M+H]⁺=200.19.

### 2-(7-Methoxy-2,3-dihydro-benzo[1,4]dioxin-6-yl)-ethylamine.HCl (AM-11.1)

Step 1: NBS (8.89 g, 50 mmol) is added portionwise to a RT soln. of 2,3-dihydro-1,4-benzodioxin-6-ol (8.0 g, 50 mmol) in DMF (80 mL) and the mix. is stirred for 2 h, before additional NBS (3.0 g) is added and the mix. is stirred for another 30 min. The mix. is diluted with water and extracted with EtOAc (3×). The combined org. extracts are washed with water (2×), brine, dried over a phase separator and concentrated. Purification by FC (eluting with 0% to 30% EtOAc in hept) yields 7-bromo-2,3-dihydro-benzo[1,4]dioxin-6-ol (5.98 g, 90%) as a red oil. LC-MS B: t_{R}=0.74 min; No ionisation.

Step 2: Mel (3.26 mL, 51.8 mmol) is added to a RT soln. of 7-bromo-2,3-dihydro-benzo[1,4]dioxin-6-ol (5.981 g, 25.9 mmol) and Cs₂CO₃ (10.12 g, 31.1 mmol) in DMF (60 mL) and the mix. is stirred for 1.5 h. The mix. is diluted with water and extracted with Et₂O (3×). The combined org. layers are washed with water, brine, dried over a phase separator, and concentrated. Purification by FC (eluting with 0% to 30% EtOAc in hept) yields 6-bromo-7-methoxy-2,3-dihydro-benzo[1,4]dioxine (5.85 g, 92%) as a white powder. LC-MS B: t_{R}=0.90 min; [M+H]⁺=244.13.

Step 3&4: The title compound is prepared from 6-bromo-7-methoxy-2,3-dihydro-benzo[1,4]dioxine following the 2-step procedure described for AM-2.1. LC-MS B: t_{R}=0.52 min; [M+H]⁺=210.24.

### 2-(7-Chloro-5-methyl-2,3-dihydro-benzo[1,4]dioxin-6-yl)-ethylamine.HCl (AM-12.1)

Step 1: 1,2-Dibromoethane (8.85 mL, 101 mmol) is added to a RT soln. of 3-methylcatechol (5.0 g, 40.3 mmol) and K₂CO₃ (22.27 g, 161 mmol) in DMF (70 mL) and the resulting mix. is stirred for 18 h. The mix. is diluted with water and the extracted with Et₂O (3×). The combined org. layers are washed with water and brine, dried over a phase separator, and concentrated. Purification by FC (eluting with 0% to 25% EtOAc in hept), yields 5-methyl-2,3-dihydro-benzo[1,4]dioxine (3.05 g, 50%) as a colourless oil. LC-MS B: t_{R}=0.83 min; No ionisation.

Step 2: NBS (3.39 g, 19.1 mmol) is added portionwise to 0° C. soln. of 5-methyl-2,3-dihydro-benzo[1,4]dioxine (2.864 g, 19.1 mmol) in THF (60 mL) and the RM is stirred at RT for 18 h. To the mix. is added NBS (286 mg) and stirring is continued for another 30 min. The mix. is diluted with water and extracted with EtOAc (3×). The combined org. extracts are washed with water (2×), brine, dried over a phase separator, and concentrated. Purification by FC (eluting with 0% to 30% EtOAc in hept), yields 6-bromo-5-methyl-2,3-dihydro-benzo[1,4]dioxine (4.40 g, 100%) as a light orange oil. LC-MS B: t_{R}=0.96 min; No ionisation.

Step 3: [2-(5-Methyl-2,3-dihydro-benzo[1,4]dioxin-6-yl)-ethyl]-carbamic acid tert-butyl ester is prepared from 6-bromo-5-methyl-2,3-dihydro-benzo[1,4]dioxine following the reaction described for AM-2.1, step 1. LC-MS B: t_{R}=0.95 min; No ionisation.

Step 4: NCS (307 mg, 2.25 mmol) is added portionwise to a RT soln. of [2-(5-methyl-2,3-dihydro-benzo[1,4]dioxin-6-yl)-ethyl]-carbamic acid tert-butyl ester (600 mg, 2.05 mmol) in DMF (10 mL) and the mix. is heated to 50° C. for 18 h.

The mixture is diluted with water and extracted with EtOAc (3×). The combined org. extracts are washed with water, brine, dried over a phase separator, and concentrated. Purification by FC (eluting with 0% to 20% EtOAc in hept) yields [2-(7-chloro-5-methyl-2,3-dihydro-benzo[1,4]dioxin-6-yl)-ethyl]-carbamic acid tert-butyl ester (581 mg, 87%) as a slightly orange oil.

Step 5: The title compound is prepared from [2-(7-chloro-5-methyl-2,3-dihydro-benzo[1,4]dioxin-6-yl)-ethyl]-carbamic acid tert-butyl ester following the reaction described for AM-2.1, step 2. LC-MS B: t_{R}=0.58 min; [M+H]⁺=228.11.

### 2-(7-Chloro-2,3-dihydro-benzo[1,4]dioxin-6-yl)-ethylamine.HCl (AM-12.2)

The title compound is prepared from 6-bromo-2,3-dihydrobenzo[b][1,4]dioxine following the 3-step sequence of reactions described for AM-12.1, step 3 to 5. LC-MS B: t_{R}=0.54 min; [M+H]⁺=214.22.

### 2-(3-Methoxyisoxazol-5-yl)ethan-1-amine hydrochloride (AM-13.1)

Step 1: DPPA (1.32 mL, 6.1 mmol) is added dropwise to a RT soln. of 3-(3-methoxyisoxazol-5-yl)propanoic acid (1.0 g, 5.55 mmol) and TEA (0.93 mL, 6.66 mmol) in PhMe (25 mL) and the RM is heated to 100° C. for 1.5 h. 2-Methylpropan-2-ol (1.06 mL, 11.1 mmol) is added and the RM is heated to reflux for 16 h. The RM is cooled to RT and partitioned between sat. aq. NaHCO₃ and EtOAc and the layers are separated. The aq. phase is re-extracted with EtOAc (2×) and the combined org. extracts are washed with brine, dried over Na₂SO₄, filtered and evaporated in vacuo. The crude product is purified by FC (eluting with 0% to 100% EtOAc in hept) to give tert-butyl (2-(3-methoxyisoxazol-5-yl)ethyl)carbamate as a colourless oil. LC-MS F: t_{R}=1.80 min; [M+H]⁺=243.1.

Step 2: The title compound is prepared from tert-butyl (2-(3-methoxyisoxazol-5-yl)ethyl)carbamate in analogy to the procedure described for AM-2.1 step 2. LC-MS B: t_{R}=0.28 min; [M+H]⁺=143.09.

### 2-(3-(Methoxy-d3)isoxazol-5-yl)ethan-1-amine hydrochloride (AM-13.2)

Step 1: H₂SO₄ (136 μL, 2.55 mmol) is added to a RT soln. of pent-4-ynoic acid (5.0 g, 51 mmol) in EtOH (50 mL) and the RM is heated to 70° C. for 2 h. The RM is partitioned between water and EtOAc and the layers are separated. The org. phase is washed with H₂O (2×) before a soln. of KHCO₃ (10.21 g, 102 mmol) in H₂O (25 mL) is added followed by the dropwise addition of a soln. of hydroxycarbonimidic dibromide (10.34 g, 51 mmol) in EtOAc (200 mL). The RM is stirred at RT for 48 h and then washed with H₂O, brine, dried over Na₂SO₄, filtered and evaporated in vacuo. The crude product is purified by FC (eluting with 1% to 15% EtOAc in hept) to give ethyl 3-(3-bromoisoxazol-5-yl)propanoate as a white solid. LC-MS F: t_{R}=1.87 min; [M+H]⁺=247.0.

Step 2: Na (1.15 g, 50 mmol) is added portionwise to methanol-d3 (11.53 mL, 285 mmol) in an ice bath and when all solids are dissolved, ethyl 3-(3-bromoisoxazol-5-yl)propanoate (1.0 g, 4.0 mmol) is added and the resulting soln. is irradiated in a MW oven at 110° C. for 75 min. The RM is diluted with H₂O and then poured into 2M HCl (35 mL) and extracted with EtOAc (3×). The combined org. extracts are washed with brine, dried over Na₂SO₄, filtered and evaporated in vacuo. The crude product is purified by FC (eluting with 5% to 10% MeOH in DCM) to give 3-(3-(methoxy-d3)isoxazol-5-yl)propanoic acid as a white solid. LC-MS F: t_{R}=1.07 min; [M+H]⁺=175.0.

Steps 3-4: The title compound is prepared from 3-(3-(methoxy-d3)isoxazol-5-yl)propanoic acid in analogy to the procedure described for AM-13.1. LC-MS F: t_{R}=0.13 min; [M+H]⁺=146.0.

### 2-(4-(3-Methoxypropyl)-1H-pyrazol-1-yl)ethan-1-amine dihydrochloride (AM-14.1)

Step 1: A mix. of carbamic acid, N-[2-(4-bromo-1H-pyrazol-1-yl)ethyl]-1,1-dimethylethyl ester (300 mg, 1.03 mmol) and trans-3-methoxy-1-propenylboronic acid pinacol ester (0.71 mL, 1.07 mmol) in dioxane (3 mL) and H₂O (3 mL) is degassed with Ar for 10 min before Cs₂CO₃ (1.18 g, 3.62 mmol) and Pd(dppf)Cl₂.DCM (25.3 mg, 0.03 mmol) are added. The RM is degassed with Ar for a further 2 min and then irradiated in a MW oven at 90° C. for 20 min (cooling function on). After cooling to RT the RM is partitioned between water and DCM and extracted. The layers are separated, and the aq. phase is re-extracted with DCM (2×). The combined org. extracts are washed with brine, dried (Mg₂SO₄), filtered and evaporated in vacuo. The crude product is purified by prep. HPLC (acidic, 5% to 95%) to give tert-butyl (2-(4-(3-methoxyprop-1-en-1-yl)-1H-pyrazol-1-yl)ethyl)carbamate (187 mg, 64%) as a colourless oil. LC-MS B: t_{R}=0.78 min; [M+H]⁺=282.29.

Step 2: Pd/C (10%, 34 mg, 0.032 mmol) is added to a RT soln. of tert-butyl (2-(4-(3-methoxyprop-1-en-1-yl)-1H-pyrazol-1-yl)ethyl)carbamate (180 mg, 0.64 mmol) in MeOH and the mix. is stirred at RT for 1 h under a H₂ atm. The mix. is filtered and concentrated to yield tert-butyl (2-(4-(3-methoxypropyl)-1H-pyrazol-1-yl)ethyl)carbamate (180 mg, 99%) as a colourless oil. LC-MS B: t_{R}=0.78 min; [M+H]⁺=284.28.

Step 3: 4 M HCl in dioxane (4.4 mL, 6.18 mmol) is added to a RT soln. of tert-butyl (2-(4-(3-methoxypropyl)-1H-pyrazol-1-yl)ethyl)carbamate (175 mg, 0.62 mmol) in dioxane (5 mL) and the resulting mix. is stirred for 30 min. The RM is concentrated in vacuo to yield the title compound AM-14.1 (157 mg, 99%) as a colourless oil. LC-MS B: t_{R}=0.42 min; [M+H]⁺=184.40.

### 2-(4-Cyclopropyl-1H-pyrazol-1-yl)ethan-1-amine dihydrochloride (AM-15.1)

Step 1: A mix. of carbamic acid, N-[2-(4-bromo-1H-pyrazol-1-yl)ethyl]-, 1,1-dimethylethyl ester (500 mg, 1.72 mmol) and cyclopropylboronic acid (459 mg, 5.34 mmol) in THF (10 mL) is degassed with Ar for 10 min before Cs₂CO₃ (1.97 g, 6.03 mmol) and Pd(dppf)Cl₂.DCM (42.2 mg, 0.052 mmol) are added. The RM is degassed with Ar for a further 2 min and then irradiated in a MW oven at 70° C. for 30 min (cooling function on). After cooling to RT the RM is partitioned between water and DCM and extracted. The layers are separated, and the aq. phase is re-extracted with DCM (2×). The combined org. extracts are washed with brine, dried (Mg₂SO₄), filtered, and evaporated in vacuo. The crude product is purified by FC (eluting with 15% to 50% EtOAc in hept, R_{f}=0.48 in EtOAc/hept 1:1) to give tert-butyl (2-(4-cyclopropyl-1H-pyrazol-1-yl)ethyl)carbamate (187 mg, 64%) as a colourless oil. LC-MS B: t_{R}=0.78 min; [M+H]⁺=282.29.

Step 2: 5 M HCl in ⁱPrOH (2.3 mL, 11.5 mmol) is added to tert-butyl (2-(4-cyclopropyl-1H-pyrazol-1-yl)ethyl)carbamate (580 mg, 2.31 mmol) at RT and the RM is stirred for 30 min. The RM is concentrated in vacuo to yield the title compound AM-15.1 (157 mg, 99%) as a colourless oil. LC-MS B: t_{R}=0.42 min; [M+H]⁺=184.40.

### 2-(4-Methoxy-1H-pyrazol-1-yl)ethan-1-amine dihydrochloride (AM-16.1)

Step 1: tert-Butyl N-(2-bromoethyl)carbamate (1.61 g, 7.06 mmol) is added to a RT suspension of 4-methoxy-1H-pyrazole hydrochloride (1.00 g, 7.06 mmol) and Cs₂CO₃ (6.97 g, 21.2 mmol) in MeCN (16.3 mL) and the RM is heated to 80° C. for 18 h. The RM is allowed to reach RT, then it is filtered and the filter cake rinsed with DCM. Purification by FC (eluting with 20% to 60% EtOAc in hept with R_{f}=0.18 (hept/EtOAc 1:1) yields tert-butyl (2-(4-methoxy-1H-pyrazol-1-yl)ethyl)carbamate (1.473 g, 87%) as a white solid.

Step 2: 4 M HCl in dioxane (13.8 mL, 55.3 mmol) is added to a 0° C. suspension of tert-butyl (2-(4-methoxy-1H-pyrazol-1-yl)ethyl)carbamate (1.47 g, 5.53 mmol) in DCM (9.5 mL) and the RM is allowed to reach RT overnight. The RM is concentrated and co-evaporated with DCM under HV to obtain the title compound AM-16.1 (1.36 g, 94%) as an off-white solid. LC-MS B: t_{R}=0.36 min; No ionisation.

### 2-(5-Cyclopropyl-2H-tetrazol-2-yl)ethan-1-amine hydrochloride (AM-16.2)

The title compound is prepared from 5-cyclopropyl-2H-1,2,3,4-tetrazole following the sequence of reactions described for AM-16.1, using K₂CO₃ instead of Cs₂CO₃. LC-MS I: t_{R}=0.44 min; [M+H]⁺=154.25.

### 2-(3-Ethynylphenyl)ethan-1-amine.TFA (AM-17.1)

Step 1: tert-Butyl (3-bromophenethyl)carbamate is prepared from 2-(3-bromophenyl)ethan-1-amine in analogy to the procedure described for AM-3.1 step 1. LC-MS B: t_{R}=1.00 min; [M+H-Me]⁺=285.12.

Step 2: A degassed mix. of tert-butyl (3-bromophenethyl)carbamate (10.51 g, 35 mmol), trimethylsilylacetylene (14.8 mL, 105 mmol), XPhos Pd G2 (1.38 g, 1.75 mmol) and TEA (14.6 mL, 105 mmol) in DMF (120 mL) is stirred at 60° C. for 18 h. The RM is partitioned between water and Et₂O and the layers are separated. The aq. phase is re-extracted with Et₂O (2×) and the combined org. extracts are washed with brine, dried over Na₂SO₄, filtered and evaporated in vacuo. The crude product is purified by FC (eluting with 0% to 60% EtOAc in hept) to give tert-butyl (3-((trimethylsilyl)ethynyl)phenethyl)carbamate as a brown oil. LC-MS B: t_{R}=1.13 min; [M+H-^{t}Bu]⁺=262.27.

Step 3: TFA (15 mL, 196 mmol) is added to a RT soln. of tert-butyl (3-((trimethylsilyl)ethynyl)phenethyl)carbamate (10.36 g, 32.6 mmol) in DCM (100 mL) and the RM is stirred for 1 h before being concentrated in vacuo. The residue is co-evaporated with DCM (2×) to give the title compound as a yellow solid. LC-MS B: t_{R}=0.49 min; [M+H]⁺=146.19.

### 2-(3-Methoxy-1,2,4-oxadiazol-5-yl)ethan-1-amine hydrochloride (AM-18.1)

Step 1: HATU (11.82 g, 31.1 mmol) is added to a RT solution of boc-beta-Ala-OH (5.0 g, 25.9 mmol), o-methylisourea bisulfate (4.5 g, 25.9 mmol, and DIPEA (18.1 mL, 104 mmol) in DMF (150 mL) and the RM is stirred at RT for 1.5 h. Water and EtOAc are added to the RM, then the two layers are separated and the inorg. layer is extracted with EtOAc (2×). The combined org. layers are washed with brine, dried (Na₂SO₄), filtered, and concentrated to give the crude product that is purified by FC (eluting with 20% to 100% EtOAc in hept) to give tert-butyl (3-((imino(methoxy)methyl)amino)-3-oxopropyl)carbamate as a white solid. LC-MS I: t_{R}=0.64 min; [M+H]⁺=246.36.

Step 2: 1,8-Diazabicyclo[5.4.0]undec-7-ene (8.96 mL, 59.3 mmol) is added to a RT soln. of tert-butyl (3-((imino(methoxy)methyl)amino)-3-oxopropyl)carbamate (6.19 g, 24.7 mmol) and NBS (10.56 g, 59.3 mmol) in EtOAc (120 mL) and the RM is stirred for 5 h. Additional 1,8-diazabicyclo[5.4.0]undec-7-ene (1.85 mL, 12.4 mmol) and NBS (2.2 g, 12.4 mmol) are added and stirring is continued for 16 h. The suspension is filtered and the filtrate is washed with water, sat. aq. NaHCO₃ soln. and brine before being evaporated to dryness. The crude product is purified by FC (eluting with 20% to 100% EtOAc in hept) to give tert-butyl (2-(3-methoxy-1,2,4-oxadiazol-5-yl)ethyl)carbamate as a colourless oil. LC-MS I: t_{R}=0.75 min; [M+H]⁺=244.33.

Step 3: 4 M HCl in dioxane (0.62 mL, 2.47 mmol) is added to a RT solution of tert-butyl (2-(3-methoxy-1,2,4-oxadiazol-5-yl)ethyl)carbamate (150 mg, 0.62 mmol) in DCM (2 mL) and the RM is stirred for 4 days at RT, then at 50° C. for 6 h. The mixture is evaporated to yield the title compound A-18.1 (79 mg, 71%) as a white solid. LC-MS I: t_{R}=0.35 min; [M+H]⁺=144.21.

### 2-(4-Cyclopropyl-2H-1,2,3-triazol-2-yl)ethan-1-amine hydrochloride (AM-19.1)

Step 1: Pd(OAc)₂ (17.1 mg, 0.076 mmol) is added to a RT solution of tert-butyl N-[2-(4-bromo-2H-1,2,3-triazol-2-yl)ethyl]carbamate (291 mg, 1 mmol), cyclopropylboronic acid (112 mg, 1.3 mmol), potassium phosphate tribasic (758 mg, 3.5 mmol), and tricyclohexylphosphine (45.1 mg, 0.156 mmol) in toluene (22 mL) and water (0.22 mL). The mixture is heated to 100° C. for 18 h. The RM is allowed to cool down to RT, then the mixture is filtered, and the filtrate concentrated. Purification by FC (eluting with 5% to 40% EtOAc in hept with R_{f}=0.27 in hept/EtOAc 7:3) yields tert-butyl (2-(4-cyclopropyl-2H-1,2,3-triazol-2-yl)ethyl)carbamate (194 mg, 77%) as a yellow oil. LC-MS B: t_{R}=0.82 min; [M+H]⁺=253.34.

Step 2: 4 M HCl in dioxane (4.9 mL, 19.6 mmol) is added to a RT solution of tert-butyl (2-(4-cyclopropyl-2H-1,2,3-triazol-2-yl)ethyl)carbamate (550 mg, 1.96 mmol) in DCM (3.4 mL). The RM is stirred at RT for 30 min, then the RM is concentrated to give title compound AM-19.1 (432 mg, 98%) as a white solid which is used as such in the next step. LC-MS B: t_{R}=0.37 min; [M+H]⁺=153.11.

### 2-(2-Aminoethyl)-2H-1,2,3-triazole-4-carbonitrile trifluoroacetate (AM-19.2)

Step 1: Vinylboronic anhydride pyridine complex (355 mg, 1.47 mmol) and 2 M aq. K₂CO₃ (2.5 mL) are added to a RT soln. of tert-butyl N-[2-(4-bromo-2H-1,2,3-triazol-2-yl)ethyl]carbamate (429 mg, 1.47 mmol) in 1,2-dimethoxyethane (7 mL) and the RM is purged with Ar for 10 min before Pd(PPh₃)₄ (34.8 mg, 0.03 mmol) is added. The mixture is heated to 80° C. for 18 h. The RM is cooled to RT, filtered, and the filtrate is partitioned between water and EtOAc and the layers are separated. The aq. phase is re-extracted with EtOAc (2×) and the combined org. extracts are washed with brine, dried over Na₂SO₄, filtered and evaporated in vacuo. The crude product is purified by prep. HPLC (acidic) to give tert-butyl (2-(4-vinyl-2H-1,2,3-triazol-2-yl)ethyl)carbamate as a brown oil. LC-MS B: t_{R}=0.81 min; [M+H]⁺=239.14.

Step 2: KMnO₄ (401 mg, 1.0 mmol) is added to a RT soln. of tert-butyl (2-(4-vinyl-2H-1,2,3-triazol-2-yl)ethyl)carbamate (118 mg, 0.50 mmol) in a 1:1 mix. of water:acetone (6 mL) and the RM is stirred for 18 h. The RM is filtered and evaporated in vacuo and the crude product is purified by prep. HPLC (acidic) to give 2-(2-((tert-butoxycarbonyl)amino)ethyl)-2H-1,2,3-triazole-4-carboxylic acid as a white solid. LC-MS B: t_{R}=0.61 min; [M+H]⁺=257.11.

Step 3: HATU (294 mg, 0.77 mmol) is added to a RT soln. of 2-(2-((tert-butoxycarbonyl)amino)ethyl)-2H-1,2,3-triazole-4-carboxylic acid (66 mg, 0.26 mmol) in DMF (1 mL) and the RM is stirred for 1 h before 25% aq. NH₃ (0.99 mL, 6.4 mmol) is added and stirring is continued for 1 h. The RM is directly purified by prep. HPLC (basic) to give tert-butyl (2-(4-carbamoyl-2H-1,2,3-triazol-2-yl)ethyl)carbamate as a white solid. LC-MS B: t_{R}=0.58 min; [M+H]⁺=256.13.

Step 4: Burgess reagent (97 mg, 0.38 mmol) is added to a RT soln. of tert-butyl (2-(4-carbamoyl-2H-1,2,3-triazol-2-yl)ethyl)carbamate (49 mg, 0.19 mmol) in DCM (2 mL) and the RM is stirred for 18 h. The RM is poured into water, diluted with DCM and extracted. The layers are separated and the aq. phase is re-extracted with DCM (2×). The combined org. extracts are washed with brine, dried over Na₂SO₄, filtered and evaporated in vacuo. The crude product is purified by prep. HPLC (acidic) to give tert-butyl (2-(4-cyano-2H-1,2,3-triazol-2-yl)ethyl)carbamate as a colourless oil. LC-MS B: t_{R}=0.80 min; [M+H]⁺=238.14.

Step 5: The title compound is prepared from tert-butyl (2-(4-cyano-2H-1,2,3-triazol-2-yl)ethyl)carbamate in analogy to the procedure described for AM-17.1 step 3. LC-MS B: t_{R}=0.22 min; [M+H]⁺=138.16.

### 2-(4-Ethynyl-5-methyl-2H-1,2,3-triazol-2-yl)ethan-1-amine (AM-19.3)

Step 1: NaH 60% dispersion in mineral oil (1.48 g, 37 mmol) is added portionwise to a 0° C. soln. of 4,5-dibromo-2H-1,2,3-triazole (4.0 g, 17.6 mmol) in DMF (120 mL) and the RM is warmed to RT and stirred for 30 min before being cooled back to 0° C. N-(2-bromoethyl)phthalimide (9.9 g, 37 mmol) is added portionwise and the RM is warmed to RT and stirred for 42 h. The RM is quenched into ice water and the precipitate is collected by filtration. The filter cake is washed with Et₂O to give 2-(2-(4,5-dibromo-2H-1,2,3-triazol-2-yl)ethyl)isoindoline-1,3-dione as a white solid. LC-MS I: t_{R}=0.98 min; No ionisation.

Step 2: ZnMe₂ (2 M in PhMe, 0.50 mL, 1.0 mmol) is added dropwise to a RT soln. of 2-(2-(4,5-dibromo-2H-1,2,3-triazol-2-yl)ethyl)isoindoline-1,3-dione (500 mg, 1.25 mmol), and Pd(dppf)Cl₂.DCM (10 mg, 0.013 mmol) in dioxane (2 mL) and the RM is heated to 70° C. and stirred for 3 h. The RM is concentrated in vacuo and the residue is partitioned between water and EtOAc and extracted. The layers are separated, and the aq. phase is re-extracted with EtOAc (2×). The combined org. extracts are washed with brine, dried over Na₂SO₄, filtered and evaporated in vacuo. The crude product is purified by prep. HPLC (basic) to give 2-(2-(4-bromo-5-methyl-2H-1,2,3-triazol-2-yl)ethyl)isoindoline-1,3-dione as a white solid. LC-MS I: t_{R}=0.92 min; No ionisation.

Step 3: A degassed mix. of 2-(2-(4-bromo-5-methyl-2H-1,2,3-triazol-2-yl)ethyl)isoindoline-1,3-dione (85 mg, 0.25 mmol), (tert-butyldimethylsilyl)acetylene (73 mg, 0.51 mmol), XPhos Pd G2 (20 mg, 0.03 mmol) and KOAc (75 mg, 0.76 mmol) in DMF (2 mL) is stirred at 70° C. for 30 min. The RM is filtered through a Whatman filter and directly purified by prep. HPLC (basic) to give 2-(2-(4-((tert-butyldimethylsilyl)ethynyl)-5-methyl-2H-1,2,3-triazol-2-yl)ethyl)isoindoline-1,3-dione as a white solid. LC-MS I: t_{R}=1.32 min; [M+H]⁺=395.29.

Step 4: Hydrazine monohydrate (0.18 mL, 2.43 mmol) is added to a RT solution of 2-(2-(4-((tert-butyldimethylsilyl)ethynyl)-5-methyl-2H-1,2,3-triazol-2-yl)ethyl)isoindoline-1,3-dione (32 mg, 0.08 mmol) in EtOH (1 mL) and the RM is heated to reflux for 2.5 h. The RM is cooled to RT and MeCN is added. The resulting suspension is filtered and the filter cake is discarded. The filtrate is concentrated in vacuo to give 2-(4-((tert-butyldimethylsilyl)ethynyl)-5-methyl-2H-1,2,3-triazol-2-yl)ethan-1-amine as a yellow oil. LC-MS I: t_{R}=1.14 min; [M+H+MeCN]⁺=306.14.

Step 5: 1 M aq. NaOH (0.25 mL, 0.25 mmol) is added to a RT soln. of 2-(4-((tert-butyldimethylsilyl)ethynyl)-5-methyl-2H-1,2,3-triazol-2-yl)ethan-1-amine (26 mg, 0.1 mmol) in EtOH (0.75 mL) and the RM is heated to 60° C. for 3 h. The RM is concentrated before water and DCM are added. The layers are separated and the aq. phase is extracted with DCM (1×). The combined org. layers are dried over a phase separator and concentrated in vacuo to give the title compound as a yellow oil. LC-MS 1: t_{R}=0.51 min; [M+H]⁺=151.22.

### 2-(4-Fluoro-3-methoxyisoxazol-5-yl)ethan-1-amine hydrochloride (AM-20.1)

Step 1: In a microwave tube, phthalic anhydride (354 mg, 2.36 mmol) is added to a RT suspension of AM-13.1 (402 mg, 2.25 mmol) and DIPEA (0.47 mL, 2.7 mmol) in dioxane (12 mL). The tube is sealed and heated to 100° C. for 48 h. Water is added to the RM, the mixture is acidified with 1 M HCl and the product extracted with EtOAc, dried (MgSO₄), filtered, and concentrated to yield 2-(2-(3-methoxyisoxazol-5-yl)ethyl)isoindoline-1,3-dione (718 mg) as a white solid which was used as such in the next step. LC-MS B: t_{R}=0.85 min; [M+H]⁺=273.09.

Step 2: Selectfluor (1.07 g, 2.87 mmol) is added to a 40° C. solution of 2-(2-(3-methoxyisoxazol-5-yl)ethyl)isoindoline-1,3-dione (710 mg, 2.61 mmol) in tetramethylene sulfone (21.7 mL, 226 mmol) and the RM is heated to 120° C. for 18 h. The resulting dark brown solution is allowed to cool down to around 50° C., then the RM is poured into pre-stirred H₂O (30 mL), followed by EtOAc (10 mL). The two layers are separated and the inorg. layer is extracted with EtOAc (5 mL). The comb. org. layers are washed with brine, dried (Na₂SO₄), filtered, and concentrated. Purification by prep HPLC (acidic) yields 2-(2-(4-fluoro-3-methoxyisoxazol-5-yl)ethyl)isoindoline-1,3-dione (89 mg, 12%) as a colorless oil. LC-MS B: t_{R}=0.90 min; [M+H]⁺=291.02.

Step 3: Hydrazine monohydrate (0.222 mL, 2.93 mmol) is added to a RT solution of 2-(2-(4-fluoro-3-methoxyisoxazol-5-yl)ethyl)isoindoline-1,3-dione (85 mg, 0.293 mmol) in EtOH (3 mL) and the RM is heated to 80° C. for 1 h. The RM is cooled down to RT and a white precipitate is formed. Ether is added and the solid (sideproduct) is triturated before filtered off. The filtrate is concentrated to yield title compound AM-20.1 (40 mg, 85%) as a colorless oil which was used as such in the next step. LC-MS B: t_{R}=0.33 min; [M+H]⁺=161.08.

### General Method 1 for the Synthesis of Building Blocks A

### tert-Butyl (S)-3-amino-4-((3-methoxyphenethyl)amino)-4-oxobutanoate (A-1.1)

Step 1: HATU (4.53 g, 11.9 mmol) is added to a RT soln. of Fmoc-L-aspartic acid beta-tert-butyl ester (5.0 g, 11.9 mmol), 3-methoxyphenethylamine (AM-1.4, 2.0 g, 13.1 mmol) and DIPEA (4.08 mL, 23.8 mmol) in DMF (40 mL) and the RM is stirred for 1 h. The RM is partitioned between water and EtOAc and the layers are separated. The aq. phase is re-extracted with EtOAc (2×) and the combined org. extracts are washed with brine, dried over Na₂SO₄, filtered and evaporated in vacuo. The crude product is purified by FC (eluting with 20% to 80% EtOAc in hept) to give tert-butyl (S)-3-((((9H-fluoren-9-yl)methoxy)carbonyl)amino)-4-((3-methoxyphenethyl)amino)-4-oxobutanoate as a white solid. LC-MS B: t_{R}=1.12 min; [M+H]⁺=545.11.

Step 2: Piperidine (4.95 mL, 49.5 mmol) is added to a RT soln. of tert-butyl (S)-3-((((9H-fluoren-9-yl)methoxy)carbonyl)amino)-4-((3-methoxyphenethyl)amino)-4-oxobutanoate (5.45 g, 9.9 mmol) in DCM (60 mL) and the RM is stirred for 2 h. The RM is concentrated in vacuo and the residue directly purified by FC (eluting with 100:2:0.5 DCM:MeOH:NH₃) to give the title compound as a colourless oil. LC-MS B: t_{R}=0.67 min; [M+H]⁺=323.34.

Listed in Table A-1 below are building blocks A that are prepared in analogy to the 2-step sequence described above for A-1.1.

TABLE A-1

| | | | t_{R} [min] | MS-data |
|---|---|---|---|---|
| | | | LC-MS | m/z |
| No. | Starting Material | Product | Method | [M + H]⁺ |
| A- | 2-(Chroman-7-yl)ethan-1- | tert-Butyl (S)-3-amino-4-((2-(chroman-7- | 0.86 | 349.21 |
| 1.2 | amine hydrochloride (AM-2.4) | yl)ethyl)amino)-4-oxobutanoate | I | |
| A- | 2-(2,3-Dihydrobenzofuran-6- | tert-Butyl (S)-3-amino-4-((2-(2,3- | 0.81 | 335.24 |
| 1.3 | yl)ethan-1-amine hydrochloride | dihydrobenzofuran-6-yl)ethyl)amino)-4- | I | |
| | (AM-2.5) | oxobutanoate | | |
| A- | 2-(4-Bromophenyl)ethan-1- | tert-Butyl (S)-3-amino-4-((4- | 0.90 | 371.13 |
| 1.4 | amine (AM-1.12) | bromophenethyl)amino)-4-oxobutanoate | I | |
| A- | 2-(2,3,4,5- | tert-Butyl (S)-3-amino-4-oxo-4-((2-(2,3,4,5- | 0.93 | 363.21 |
| 1.5 | Tetrahydrobenzo[b]oxepin-8- | tetrahydrobenzo[b]oxepin-8- | I | |
| | yl)ethan-1-amine hydrochloride | yl)ethyl)amino)butanoate | | |
| | (AM-2.21) | | | |
| A- | 2-(2,3- | tert-Butyl (S)-3-amino-4-((2-(2,3- | 0.66 | 351.47 |
| 1.6 | Dihydrobenzo[b][1,4]dioxin-6- | dihydrobenzo[b][1,4]dioxin-6- | B | |
| | yl)ethan-1-amine hydrochloride | yl)ethyl)amino)-4-oxobutanoate | | |
| | (AM-2.6) | | | |
| A- | 2-(4-(2H-1,2,3-Triazol-2- | tert-Butyl (S)-4-((4-(2H-1,2,3-triazol-2- | 0.69 | 360.45 |
| 1.7 | yl)phenyl)ethan-1-amine | yl)phenethyl)amino)-3-amino-4- | B | |
| | hydrochloride (AM2.7) | oxobutanoate | | |
| A- | 2-(3-Methylisoxazol-5-yl)ethan- | tert-Butyl (S)-3-amino-4-((2-(3- | 0.64 | 298.26 |
| 1.8 | 1-amine hydrochloride | methylisoxazol-5-yl)ethyl)amino)-4- | I | |
| | (AM-1.40 | oxobutanoate | | |
| A- | 2-(3-Bromophenyl)ethan-1- | tert-Butyl (S)-3-amino-4-((3- | 0.73 | 371.36 |
| 1.9 | amine | bromophenethyl)amino)-4-oxobutanoate | B | |
| A- | 2-(3- | tert-Butyl (S)-3-amino-4-((3- | 0.73 | 359.45 |
| 1.10 | (Difluoromethoxy)phenyl)ethan- | (difluoromethoxy)phenethyl)amino)-4- | B | |
| | 1-amine (AM-1.57) | oxobutanoate | | |
| A- | 2-(5,6-Dihydro-4H- | tert-Butyl (S)-3-amino-4-((2-(5,6-dihydro-4H- | 0.64 | 340.15 |
| 1.11 | cyclopenta[d]thiazol-2-yl)ethan- | cyclopenta[d]thiazol-2-yl)ethyl)amino)-4- | B | |
| | 1-amine (AM-1.49) | oxobutanoate | | |
| A- | 2-(3-Ethoxyphenyl)ethan-1- | tert-Butyl (S)-3-amino-4-((3- | 0.90 | 337.25 |
| 1.12 | amine (AM-1.58) | ethoxyphenethyl)amino)-4-oxobutanoate | I | |
| A- | (RS)-2-(5,5- | tert-Butyl (S)-3-amino-4-((2-((RS)-5,5- | 0.88 | 329.27 |
| 1.13 | Dimethyltetrahydro-2H-pyran- | dimethyltetrahydro-2H-pyran-2- | I | |
| | 2-yl)ethan-1-amine (AM-1.59) | yl)ethyl)amino)-4-oxobutanoate | | |
| A- | 2-(Benzo[d][1,3]dioxol-5- | tert-Butyl (S)-3-amino-4-((2- | 0.67 | 337.20 |
| 1.14 | yl)ethan-1-amine hydrochloride | (benzo[d][1,3]dioxol-5-yl)ethyl)amino)-4- | B | |
| | | oxobutanoate | | |
| A- | 2-(3,5-Dimethoxyphenyl)ethan- | tert-Butyl (S)-3-amino-4-((3,5- | 0.69 | 353.20 |
| 1.15 | 1-amine (AM-1.7) | dimethoxyphenethyl)amino)-4-oxobutanoate | B | |
| A- | 2-(Benzo[d]oxazol-2-yl)ethan- | tert-Butyl (S)-3-amino-4-((2- | 0.65 | 334.35 |
| 1.16 | 1-amine hydrochloride | (benzo[d]oxazol-2-yl)ethyl)amino)-4- | B | |
| | (AM-1.3) | oxobutanoate | | |
| A- | 2-(3-(2H-1,2,3-Triazol-2- | tert-Butyl (S)-4-((3-(2H-1,2,3-triazol-2- | 0.69 | 360.51 |
| 1.17 | yl)phenyl)ethan-1-amine | yl)phenethyl)amino)-3-amino-4- | B | |
| | hydrochloride (AM-2.8) | oxobutanoate | | |
| A- | 2-(5-Cyclopropylisoxazol-3- | tert-Butyl (S)-3-amino-4-((2-(5- | 0.76 | 324.27 |
| 1.18 | yl)ethan-1-amine (AM-1.33) | cyclopropylisoxazol-3-yl)ethyl)amino)-4- | I | |
| | | oxobutanoate | | |
| A- | 2-(5-Cyclopropyl-1,2,4- | tert-Butyl (S)-3-amino-4-((2-(5-cyclopropyl- | 0.70 | 325.31 |
| 1.19 | oxadiazol-3-yl)ethan-1-amine | 1,2,4-oxadiazol-3-yl)ethyl)amino)-4- | I | |
| | hydrochloride (AM-1.26) | oxobutanoate | | |
| A- | 2-(2,6-Difluorophenyl)ethan-1- | tert-Butyl (S)-3-amino-4-((2,6- | 0.87 | 329.26 |
| 1.20 | amine (AM-1.22) | difluorophenethyl)amino)-4-oxobutanoate | I | |
| A- | 2-(4-(Pyrimidin-2- | tert-Butyl (S)-3-amino-4-oxo-4-((4- | 0.66 | 371.22 |
| 1.21 | yl)phenyl)ethan-1-amine | (pyrimidin-2-yl)phenethyl)amino)butanoate | B | |
| | hydrochloride (AM-2.12) | | | |
| A- | 2-(3-Cyclopropyl-1,2,4- | tert-Butyl (S)-3-amino-4-((2-(3-cyclopropyl- | 0.72 | 325.29 |
| 1.22 | oxadiazol-5-yl)ethan-1-amine | 1,2,4-oxadiazol-5-yl)ethyl)amino)-4- | I | |
| | hydrochloride (AM-1.35) | oxobutanoate | | |
| A- | 2-(3-(Methoxy- | tert-Butyl (S)-3-amino-4-((3-(methoxy- | 0.82 | 326.33 |
| 1.23 | d3)phenyl)ethan-1-amine | d3)phenethyl)amino)-4-oxobutanoate | I | |
| | (AM-3.2) | | | |
| A- | 2-(3-Cyclopropylisoxazol-5- | tert-Butyl (S)-3-amino-4-((2-(3- | 0.73 | 324.31 |
| 1.24 | yl)ethan-1-amine hydrochloride | cyclopropylisoxazol-5-yl)ethyl)amino)-4- | I | |
| | (AM-5.2) | oxobutanoate | | |
| A- | 4-Ethylphenethylamine | tert-Butyl (S)-3-amino-4-((4- | 0.98 | 321.38 |
| 1.25 | (AM-1.10) | ethylphenethyl)amino)-4-oxobutanoate | I | |
| A- | 2-(3-(1,1- | tert-Butyl (S)-3-amino-4-((3-(1,1- | 2.0 | 357.1 |
| 1.26 | Difluoroethyl)phenyl)ethan-1- | difluoroethyl)phenethyl)amino)-4- | J | |
| | amine (AM-2.22) | oxobutanoate | | |
| A- | 2-(3-(Pyrimidin-2- | tert-butylButyl (S)-3-amino-4-oxo-4-((3- | 0.66 | 371.23 |
| 1.27 | yl)phenyl)ethan-1-amine | (pyrimidin-2-yl)phenethyl)amino)butanoate | B | |
| | dihydrochloride (AM-2.11) | | | |
| A- | 2-(3-Methoxyisoxazol-5- | tert-Butyl (S)-3-amino-4-((2-(3- | 0.68 | 314.36 |
| 1.28 | yl)ethan-1-amine hydrochloride | methoxyisoxazol-5-yl)ethyl)amino)-4- | I | |
| | (AM-13.1) | oxobutanoate | | |
| A- | 2-(3-(Methoxy-d3)isoxazol-5- | tert-Butyl (S)-3-amino-4-((2-(3-(methoxy- | 0.67 | 317.39 |
| 1.29 | yl)ethan-1-amine hydrochloride | d3)isoxazol-5-yl)ethyl)amino)-4- | I | |
| | (AM-13.2) | oxobutanoate | | |
| A- | 2-(2-Cyclopropyl-2H-1,2,3- | tert-Butyl (S)-3-amino-4-((2-(2-cyclopropyl- | 0.69 | 324.36 |
| 1.30 | triazol-4-yl)ethan-1-amine | 2H-1,2,3-triazol-4-yl)ethyl)amino)-4- | I | |
| | hydrochloride (AM-2.23) | oxobutanoate | | |
| A- | 2-(3-(Trifluoromethyl)isoxazol- | tert-Butyl (S)-3-amino-4-oxo-4-((2-(3- | 0.85 | 352.23 |
| 1.31 | 5-yl)ethan-1-amine (AM-1.84) | (trifluoromethyl)isoxazol-5- | I | |
| | | yl)ethyl)amino)butanoate | | |
| A- | 2-(3-(Difluoromethyl)isoxazol-5- | tert-Butyl (S)-3-amino-4-((2-(3- | 0.75 | 334.32 |
| 1.32 | yl)ethan-1-amine (AM-5.3) | (difluoromethyl)isoxazol-5-yl)ethyl)amino)-4- | I | |
| | | oxobutanoate | | |
| A- | But-3-yn-1-amine hydrochloride | tert-Butyl (S)-3-amino-4-(but-3-yn-1- | 1.63 | 185.1 |
| 1.33 | (AM-1.86) | ylamino)-4-oxobutanoate | J | (-^{t}Bu) |
| A- | Benzyl 3-aminopropanoate 4- | tert-Butyl (S)-3-amino-4-((3-(benzyloxy)-3- | 1.84 | 351.2 |
| 1.34 | methylbenzenesulfonate | oxopropyl)amino)-4-oxobutanoate | J | |
| | (AM-1.87) | | | |
| A- | (S)-2-Amino-1-phenylethan-1- | tert-Butyl (S)-3-amino-4-(((S)-2-hydroxy-2- | 0.70 | 309.31 |
| 1.35 | ol (AM-1.88) | phenylethyl)amino)-4-oxobutanoate | I | |
| A- | 2-(3-Methoxy-1,2,4-oxadiazol- | tert-Butyl (S)-3-amino-4-((2-(3-methoxy- | 0.62 | 315.44 |
| 1.36 | 5-yl)ethan-1-amine | 1,2,4-oxadiazol-5-yl)ethyl)amino)-4- | I | |
| | hydrochloride (AM-18.1) | oxobutanoate | | |
| A- | 2-(5-Cyclopropyl-2H-tetrazol-2- | tert-Butyl (S)-3-amino-4-((2-(5-cyclopropyl- | 0.96 | 325.47 |
| 1.37 | yl)ethan-1-amine hydrochloride | 2H-tetrazol-2-yl)ethyl)amino)-4- | I | |
| | (AM-16.2) | oxobutanoate | | |
| A- | 2-(4-Cyclopropyl-2H-1,2,3- | tert-Butyl (S)-3-amino-4-((2-(4-cyclopropyl- | 0.59 | 324.03 |
| 1.38 | triazol-2-yl)ethan-1-amine | 2H-1,2,3-triazol-2-yl)ethyl)amino)-4- | B | |
| | hydrochloride (AM-19.1) | oxobutanoate | | |
| A- | 2-Amino-1-(3-cyclopropyl- | tert-Butyl (S)-3-amino-4-(((R)-2-(3- | 0.53 | 341.28 |
| 1.39 | 1,2,4-oxadiazol-5-yl)ethan-1-ol | cyclopropyl-1,2,4-oxadiazol-5-yl)-2- | A | |
| | (AM-1.90) | hydroxyethyl)amino)-4-oxobutanoate | | |
| | | (chiral separation before F-moc | | |
| | | deprotection) | | |
| A- | 2-Amino-1-(3-cyclopropyl- | tert-Butyl (S)-3-amino-4-(((S)-2-(3- | 0.53 | 341.23 |
| 1.40 | 1,2,4-oxadiazol-5-yl)ethan-1-ol | cyclopropyl-1,2,4-oxadiazol-5-yl)-2- | A | |
| | (AM-1.90) | hydroxyethyl)amino)-4-oxobutanoate | | |
| | | (chiral separation before F-moc | | |
| | | deprotection) | | |
| A- | 2-(2H-Benzo[d][1,2,3]triazol-2- | tert-Butyl (S)-4-((2-(2H- | 0.61 | 334.26 |
| 1.41 | yl)ethan-1-amine (AM-1.91) | benzo[d][1,2,3]triazol-2-yl)ethyl)amino)-3- | A | |
| | | amino-4-oxobutanoate | | |
| A- | 3-(Trifluoromethoxy)propan-1- | tert-Butyl (S)-3-amino-4-oxo-4-((3- | 0.63 | 315.24 |
| 1.42 | amine (AM-1.92) | (trifluoromethoxy)propyl)amino)butanoate | A | |
| A- | 3-(Difluoromethoxy)propan-1- | tert-Butyl (S)-3-amino-4-((3- | 0.59 | 297.28 |
| 1.43 | amine (AM-1.45) | (difluoromethoxy)propyl)amino)-4- | A | |
| | | oxobutanoate | | |
| A- | 2-([1,2,4]Triazolo[1,5- | tert-Butyl (S)-4-((2-([1,2,4]triazolo[1,5- | 0.46 | 335.33 |
| 1.44 | a]pyrimidin-2-yl)ethan-1-amine | a]pyrimidin-2-yl)ethyl)amino)-3-amino-4- | A | |
| | (AM-1.93) | oxobutanoate | | |
| A- | 2-(1-(2-Methoxyethyl)-1H- | tert-Butyl (S)-3-amino-4-((2-(1-(2- | 0.53 | 341.31 |
| 1.45 | pyrazol-4-yl)ethan-1-amine | methoxyethyl)-1H-pyrazol-4-yl)ethyl)amino)- | A | |
| | (AM-1.94) | 4-oxobutanoate | | |
| A- | 2-(4-Fluoro-3-methoxyisoxazol- | tert-Butyl (S)-3-amino-4-((2-(4-fluoro-3- | 0.71 | 332.50 |
| 1.46 | 5-yl)ethan-1-amine | methoxyisoxazol-5-yl)ethyl)amino)-4- | I | |
| | hydrochloride | oxobutanoate | | |
| | (AM-20.1) | | | |

### General Method 2 for the Synthesis of Building Blocks A

### 4-Allyl 1-benzyl L-aspartate hydrochloride (A-2.1)

Step 1: Benzyl bromide (2.15 mL, 17.7 mmol) is added to a RT mix. of Boc-L-aspartic acid-beta-allyl ester (5.0 g, 17.7 mmol) and KHCO₃ (1.8 g, 17.7 mmol) in DMF (30 mL) and the RM is stirred for 16 h. The RM is concentrated in vacuo and the residue is partitioned between H₂O and EtOAC and extracted. The layers are separated and the aq. phase is re-extracted with EtOAc (2×). The combined org. extracts are washed with brine, dried over Na₂SO₄, filtered and evaporated in vacuo to give 4-allyl 1-benzyl (tert-butoxycarbonyl)-L-aspartate as a yellow oil. LC-MS B: t_{R}=1.01 min; [M+H]⁺=364.45.

Step 2: 4M HCl in dioxane (17.7 mL, 71 mmol) is added to a soln. of 4-allyl 1-benzyl (tert-butoxycarbonyl)-L-aspartate (6.43 g, 17.7 mmol) in dioxane (40 mL) and the RM is stirred for 5 h at RT. The volatiles are removed in vacuo and the residue is triturated with Et₂O to give the title compound as a white solid. LC-MS B: t_{R}=0.61 min; [M+H]⁺=264.34.

### Allyl (S)-3-amino-4-((2-(benzo[d][1,3]dioxol-5-yl)ethyl)amino)-4-oxobutanoate hydrochloride (A-2.2)

Step 1: Allyl (S)-4-((2-(benzo[d][1,3]dioxol-5-yl)ethyl)amino)-3-((tert-butoxycarbonyl)amino)-4-oxobutanoate is prepared from (S)-4-(allyloxy)-2-((tert-butoxycarbonyl)amino)-4-oxobutanoic acid in analogy to the procedure described for A-1.1 step 1. LC-MS B: t_{R}=0.94 min; [M+H]⁺=421.16.

Step 2: The title compound is prepared from (S)-4-(allyloxy)-2-((tert-butoxycarbonyl)amino)-4-oxobutanoic acid in analogy to the procedure described for A-2.1 step 2. LC-MS B: t_{R}=0.61 min; [M+H]⁺=321.20.

Listed in Table A-2 below are building blocks A that are prepared in analogy to the 2-step sequence described above for A-2.2.

TABLE A-2

| | | | t_{R} [min] | MS-data |
|---|---|---|---|---|
| | | | LC-MS | m/z |
| No. | Starting Material | Product | Method | [M + H]⁺ |
| A-2.3 | (S)-4-(Allyloxy)-2-((tert- | Allyl (S)-3-amino-4-((3- | 0.74 | 307.21 |
| | butoxycarbonyl)amino)-4- | methoxyphenethyl)amino)-4- | I | |
| | oxobutanoic acid | oxobutanoate hydrochloride | | |
| A-2.4 | (S)-4-(Allyloxy)-2-((tert- | Allyl (S)-3-amino-4-((2-(3- | 0.57 | 282.21 |
| | butoxycarbonyl)amino)-4- | methylisoxazol-5-yl)ethyl)amino)-4- | I | |
| | oxobutanoic acid | oxobutanoate hydrochloride | | |

### General Method 1 for the Synthesis of Building Blocks B

### Benzyl (R)-2-(2-amino-3-phenylpropoxy)-4,6-dimethoxybenzoate hydrochloride (B-1.1)

Step 1: KHCO₃ (1.6 g, 15.8 mmol) and benzyl bromide (2.1 mL, 17.3 mmol) are added to a soln. of 2-hydroxy-4,6-dimethoxybenzoic acid (3 g, 14.4 mmol) in DMF (40 mL) and the RM is stirred for 16 h. The RM is filtered and the filtrate concentrated in vacuo. The residue is partitioned between water and EtOAc and extracted. The layers are separated, and the aq. phase is re-extracted with EtOAc (2×). The combined org. extracts are washed with brine, dried over Na₂SO₄, filtered and evaporated in vacuo. The crude product is purified by FC (eluting with 5% to 40% EtOAc in hept) to give benzyl 2-hydroxy-4,6-dimethoxybenzoate as a white solid. LC-MS B: t_{R}=1.04 min; [M+H]⁺=289.23.

Step 2: DIAD (3.0 mL, 15 mmol) is added to a 0° C. mix. of 2-hydroxy-4,6-dimethoxybenzoate (3.16 g, 10.7 mmol), tert-butyl (R)-(1-hydroxy-3-phenylpropan-2-yl)carbamate (4.12 g, 16.1 mmol), and PPh₃ (4.27 g, 16.1 mmol) in THE (40 mL) and the RM is stirred for 16 h at RT. The mix. is concentrated and the residue directly purified by FC (eluting with 20% to 100% EtOAc in hept) to give benzyl (R)-2-(2-((tert-butoxycarbonyl)amino)-3-phenylpropoxy)-4,6-dimethoxybenzoate as a colourless oil. LC-MS B: t_{R}=1.16 min; [M+H]⁺=522.26.

Step 3: 4M HCl in dioxane (21 mL, 86.3 mmol) is added to a soln. of benzyl (R)-2-(2-((tert-butoxycarbonyl)amino)-3-phenylpropoxy)-4,6-dimethoxybenzoate (4.64 g, 8.63 mmol) in dioxane (40 mL) and the RM is stirred for 5 h at RT. The volatiles are removed in vacua and the residue is triturated with Et₂ (3×) to give the title compound as a white solid. LC-MS B: t_{R}=0.84 min; [M+H]⁺=422.36.

Listed in Table B-1 below are building blocks B that are prepared in analogy to the 3-step sequence described above for B-1.1. In cases where the HCl salt is highly hygroscopic, the amine is subjected to a basic workup to liberate its free base.

TABLE B-1

| | | | t_{R} [min] | MS-data |
|---|---|---|---|---|
| | | | LC-MS | m/z |
| No. | Starting Material | Product | Method | [M + H]⁺ |
| B-1.2 | 2-Hydroxy-6-methylbenzoic | Benzyl (R)-2-(2-amino-3- | 2.28 | 376.2 |
| | acid | phenylpropoxy)-6-methylbenzoate | J | |
| B-1.3 | 2-Hydroxy-5-methylbenzoic | Benzyl (R)-2-(2-amino-3- | 1.83 | 376.2 |
| | acid | phenylpropoxy)-5-methylbenzoate | F | |
| B-1.4 | 2-Hydroxy-4-methylbenzoic | Benzyl (R)-2-(2-amino-3- | 1.83 | 376.2 |
| | acid | phenylpropoxy)-4-methylbenzoate | F | |
| B-1.5 | 2-Hydroxy-6- | Benzyl (R)-2-(2-amino-3- | 1.06 | 392.14 |
| | methoxybenzoic acid | phenylpropoxy)-6-methoxybenzoate | I | |
| | | hydrochloride | | |
| B-1.6 | 2-Hydroxy-5- | Benzyl (R)-2-(2-amino-3- | 2.26 | 392.2 |
| | methoxybenzoic acid | phenylpropoxy)-5-methoxybenzoate | J | |
| B-1.7 | 2-Hydroxy-4- | Benzyl (R)-2-(2-amino-3- | 2.88 | 392.2 |
| | methoxybenzoic acid | phenylpropoxy)-4-methoxybenzoate | F | |
| B-1.8 | 5-Chloro-2-hydroxybenzoic | Benzyl (R)-2-(2-amino-3- | 1.80 | 396.1 |
| | acid | phenylpropoxy)-5-chlorobenzoate | F | |
| B-1.9 | 5-Fluoro-2-hydroxybenzoic | Benzyl (R)-2-(2-amino-3- | 2.29 | 380.2 |
| | acid | phenylpropoxy)-5-fluorobenzoate | J | |
| B-1.10 | 4-Hydroxy-6-methylnicotinic | Benzyl (R)-4-(2-amino-3- | 2.08 | 377.1 |
| | acid | phenylpropoxy)-6-methylnicotinate | J | |
| | | dihydrochloride | | |
| B-1.11 | 2-Hydroxy-4,5- | Benzyl (R)-2-(2-amino-3- | 1.75 | 422.2 |
| | dimethoxybenzoic acid | phenylpropoxy)-4,5- | F | |
| | | dimethoxybenzoate | | |
| B-1.12 | 3-Hydroxy-2-naphthoic acid | Benzyl (R)-3-(2-amino-3- | 1.83 | 412.2 |
| | | phenylpropoxy)-2-naphthoate | F | |
| | | hydrochloride | | |
| B-1.13 | 2-Hydroxy-1-naphthoic acid | Benzyl (R)-2-(2-amino-3- | 0.88 | 412.37 |
| | | phenylpropoxy)-1-naphthoate | B | |
| | | hydrochloride | | |
| B-1.14 | 3-Hydroxyquinoline-4- | Benzyl (R)-3-(2-amino-3- | 0.83 | 413.34 |
| | carboxylic acid | phenylpropoxy)quinoline-4- | B | |
| | | carboxylate dihydrochloride | | |
| B-1.16 | 6-Hydroxy-2H-1,3- | Benzyl (R)-6-(2-amino-3- | 0.84 | 406.13 |
| | benzodioxole-5-carboxylic | phenylpropoxy)benzo[d][1,3]dioxole- | B | |
| | acid | 5-carboxylate hydrochloride | | |
| B-1.19 | 6-Fluoro-3-hydroxyquinoline- | Benzyl (R)-3-(2-amino-3- | 1.12 | 431.24 |
| | 4-carboxylic acid | phenylpropoxy)-6-fluoroquinoline-4- | I | |
| | | carboxylate dihydrochloride | | |
| B-1.20 | 8-Fluoro-3-hydroxyquinoline- | Benzyl (R)-3-(2-amino-3- | 0.83 | 431.19 |
| | 4-carboxylic acid | phenylpropoxy)-8-fluoroquinoline-4- | B | |
| | | carboxylate dihydrochloride | | |
| B-1.21 | 2-Fluoro-6-hydroxybenzoic | Benzyl (R)-2-(2-amino-3- | 0.83 | 380.15 |
| | acid | phenylpropoxy)-6-fluorobenzoate | B | |
| | | hydrochloride | | |
| B-1.22 | 2-Hydroxy-4- | Benzyl (R)-2-(2-amino-3- | 0.87 | 392.27 |
| | methoxybenzoic acid | phenylpropoxy)-4-methoxybenzoate | B | |
| | | hydrochloride | | |
| B-1.23 | 3-Hydroxyquinoline-4- | Benzyl (S)-3-(2-amino-3- | 1.07 | 413.24 |
| | carboxylic acid | phenylpropoxy)quinoline-4- | I | |
| | | carboxylate dihydrochloride | | |
| B-1.26 | 6-Chloro-3-hydroxyquinoline- | Benzyl (R)-3-(2-amino-3- | 0.94 | 447.09 |
| | 4-carboxylic acid | phenylpropoxy)-6-chloroquinoline-4- | D | |
| | | carboxylate dihydrochloride | | |

### Benzyl (R)-2-(2-amino-3-phenylpropoxy)-6-ethoxybenzoate hydrochloride (B-1.17)

Step 1: Benzyl bromide (0.92 mL, 7.6 mmol) is added to a RT mix. of 2,6-dihydroxybenzoic acid (1.0 g. 6.3 mmol) and NaHCO₃ (582 mg, 6.9 mmol) in DMF (16 mL) and the RM is heated to 60° C. for 6 h. The RM is partitioned between water and EtOAc and extracted. The layers are separated, and the aq. phase is re-extracted with EtOAc (2×). The combined org. extracts are washed with brine, dried over Na₂SO₄, filtered and evaporated in vacuo. The crude product is purified by FC (eluting with 0% to 10% EtOAc in hept) to give benzyl 2,6-dihydroxybenzoate as a colourless oil. LC-MS B: t_{R}=0.98 min; [M+H]⁺=245.35.

Step 2: Ethyl iodide (0.086 mL, 1.1 mmol) is added to a RT mix. of benzyl 2,6-dihydroxybenzoate (376 mg, 1.1 mmol) and Cs₂CO₃ (351 mg, 1.1 mmol) in DMF (15 mL) and the RM is stirred for 16 h. The RM is partitioned between water and EtOAc and the layers are separated. The aq. phase is re-extracted with EtOAc (2×) and the combined org. layers are washed with brine, dried over Na₂SO₄, filtered and evaporated in vacuo. The crude product is purified by FC (eluting with 0% to 10% EtOAc in hept) to give benzyl 2-ethoxy-6-hydroxybenzoate as a yellow solid. LC-MS B: t_{R}=1.01 min; [M+H]⁺=273.29.

Steps 3-4: The title compound is prepared from benzyl 2-ethoxy-6-hydroxybenzoate following steps 2&3 described for B-1.1. LC-MS B: t_{R}=0.85 min; [M+H]⁺=406.40.

### Benzyl (R)-4-(2-amino-3-phenylpropoxy)-2-methoxynicotinate (B-1.18)

Step 1: CDI (615 mg, 3.8 mmol) is added to a soln. of 4-hydroxy-2-methoxynicotinic acid (452 mg, 2.7 mmol) in DMF (5 mL) and the RM is heated to 60° C. for 2 h. After cooling to 0° C., additional DMF (5 mL), benzyl alcohol (0.5 mL, 4.8 mmol), and NaH (118 mg, 2.95 mmol) are added and the RM is warmed to RT and stirred for 16 h. The RM is partitioned between 1N HCl and DCM and the layers are separated. The aq. phase is re-extracted with DCM (1×) and the combined org. layers are washed with brine, dried over Na₂SO₄, filtered and evaporated in vacuo. The crude product is purified by FC (eluting with 0% to 35% EtOAc in hept) to give benzyl 4-hydroxy-2-methoxynicotinate as a white solid. LC-MS F: t_{R}=2.07 min; [M+H]⁺=260.0.

Step 2: Benzyl (R)-4-(2-((tert-butoxycarbonyl)amino)-3-phenylpropoxy)-2-methoxynicotinate is prepared from benzyl 4-hydroxy-2-methoxynicotinate and tert-butyl (R)-(1-hydroxy-3-phenylpropan-2-yl)carbamate in analogy to the procedure described for B-1.1 step 2. LC-MS J: t_{R}=2.34 min; [M+H]⁺=493.2.

Step 3: TFA (3.65 mL, 4.77 mmol) is added to a 0° C. soln. of benzyl (R)-4-(2-((tert-butoxycarbonyl)amino)-3-phenylpropoxy)-2-methoxynicotinate (235 mg, 0.48 mmol) in DCM (6 mL) and the RM is warmed to RT and stirred for 3 h. The RM is concentrated in vacuo and the residue partitioned between DCM and sat. aq. NaHCO₃ and the layers are separated. The aq. phase is re-extracted with DCM (1×) and the combined org. layers are washed with brine, dried over Na₂SO₄, filtered and evaporated in vacuo to give the title compound as a yellow oil. LC-MS I: t_{R}=1.01 min; [M+H]⁺=393.15.

### (R)-2-(2-((tert-Butoxycarbonyl)amino)-3-(pyridin-2-yl)propoxy)-1-naphthoic acid (B-1.24)

Step 1: Benzyl 2-hydroxy-1-naphthoate is prepared from 2-hydroxy-1-naphthoic acid in analogy to the procedure described for B-1.1 step 1. LC-MS B: t_{R}=1.08 min; [M+H]⁺=279.51.

Step 2: (R)-2-((tert-Butoxycarbonyl)amino)-3-(pyridin-2-yl)propanoic acid (500 mg, 1.8 mmol) is added to a 0° C. suspension of LAH (85 mg, 2.2 mmol) in Et₂O (13 mL) and the RM is warmed to RT and stirred for 1 h. The RM is cooled to 0° C. and quenched with EtOAc before a sat. aq. Rochelle's salt soln. is added and vigorous stirring is maintained for 30 min after which the layers are separated. The aq. phase is re-extracted with EtOAc (3×) and the combined org. layers are washed with brine, dried over Na₂SO₄, filtered and evaporated in vacuo to give tert-butyl (R)-(1-hydroxy-3-(pyridin-2-yl)propan-2-yl)carbamate as a yellow oil. LC-MS I: t_{R}=0.63 min; [M+H]⁺=253.22.

Step 3: Benzyl (R)-2-(2-((tert-butoxycarbonyl)amino)-3-(pyridin-2-yl)propoxy)-1-naphthoate is prepared from the products of steps 1&2 above in analogy to the procedure described for B-1.1 step 2. LC-MS I: t_{R}=1.23 min; [M+H]⁺=514.18.

Step 4: A soln. of benzyl (R)-2-(2-((tert-butoxycarbonyl)amino)-3-(pyridin-2-yl)propoxy)-1-naphthoate (108 mg, 0.17 mmol) in MeOH (3 mL) is evacuated/purged with N₂ (3×) before 10% Pd/C (18 mg, 10 mol %) is added. The RM is evacuated/purged with H₂ (3×) and stirred under a H₂ atm for 2 h. The RM is filtered through a pad of celite and the filtrate concentrated in vacuo to give the title compound as a white solid. LC-MS I: t_{R}=0.48 min; [M+H]⁺=423.23.

### (R)-3-(3-(Benzo[b]thiophen-3-yl)-2-((tert-butoxycarbonyl)amino)propoxy)quinoline-4-carboxylic acid (B-1.25)

Step 1: A soln. of TMS-diazomethane (2 M in Et₂O, 0.6 mL, 1.2 mmol) is added to a 0° C. soln. of (R)-3-(benzo[b]thiophen-3-yl)-2-((tert-butoxycarbonyl)amino)propanoic acid (128 mg, 0.4 mmol) in MeOH (2 mL) and the RM is warmed to RT and stirred for 2 h. The RM is subsequently purified by prep. HPLC (basic) to furnish methyl (R)-3-(benzo[b]thiophen-3-yl)-2-((tert-butoxycarbonyl)amino)propanoate as a white solid. LC-MS I: t_{R}=1.08 min; [M+H]⁺=336.25.

Step 2: NaBH₄ (30 mg, 0.78 mmol) is added to a 0° C. mix. of methyl (R)-3-(benzo[b]thiophen-3-yl)-2-((tert-butoxycarbonyl)amino)propanoate (130 mg, 0.39 mmol) in EtOH (1 mL) and H₂O (1 mL) and the RM is warmed to RT and stirred for 16 h. The RM is subsequently purified by prep. HPLC (basic) to furnish tert-butyl (R)-(1-(benzo[b]thiophen-3-yl)-3-hydroxypropan-2-yl)carbamate as a colourless oil. LC-MS I: t_{R}=0.97 min; [M+H]⁺=308.16.

Step 3: Benzyl 3-hydroxyquinoline-4-carboxylate is prepared from 3-hydroxyquinoline-4-carboxylic acid in analogy to the procedure described for B-1.1 step 1. LC-MS B: t_{R}=0.95 min; [M+H]⁺=280.19.

Step 4: Benzyl (R)-3-(3-(benzo[b]thiophen-3-yl)-2-((tert-butoxycarbonyl)amino)propoxy)quinoline-4-carboxylate is prepared from the products of steps 2&3 above in analogy to the procedure described for B-1.1 step 2. LC-MS I: t_{R}=0.85 min; [M+H]⁺=569.24.

Step 5: LiOH·H₂O (20 mg, 0.48 mmol) is added to a mix. of benzyl (R)-3-(3-(benzo[b]thiophen-3-yl)-2-((tert-butoxycarbonyl)amino)propoxy)quinoline-4-carboxylate (136 mg, 0.24 mmol) in 2:1 THF:H₂O (3 mL) and the RM is heated to 50° C. for 16 h. The RM is concentrated in vacuo and the residue is partitioned between 1M aq. HCl and EtOAc and extracted. The layers are separated, and the aq. phase is re-extracted with EtOAc (2×). The combined org. extracts are washed with brine, dried over Na₂SO₄, filtered and evaporated in vacuo to give the title compound as a yellow solid. LC-MS B: t_{R}=0.96 min; [M+H]⁺=479.23.

### Benzyl (R)-3-(2-amino-3-phenylpropoxy)-5-fluoroquinoline-4-carboxylate dihydrochloride (B-1.27)

Step 1: 4-Fluoroindoline-2,3-dione (4.8 g, 27.6 mmol) is added to a RT soln. of KOH (18.6 g, 331 mmol) in water (80 mL) followed by bromopyruvic acid (6.47 g, 38 mmol) and the RM is stirred for 16 h. Additional KOH (4.6 g, 83 mmol) and bromopyruvic acid (2.88 g, 17.3 mmol) are added and stirring is continued for another 16 h. The RM is poured into 1M aq. HCl and the resulting precipitate is collected by filtration and washed with water and EtOAc and then dried in a vacuum oven at 40° C. to give 5-fluoro-3-hydroxyquinoline-4-carboxylic acid as a brown solid. LC-MS B: t_{R}=0.39 min; [M+H]⁺=208.09.

Steps 2-4: The title compound is prepared from 5-fluoro-3-hydroxyquinoline-4-carboxylic acid following the sequence of reactions as described for B-1.1. LC-MS B: t_{R}=0.83 min; [M+H]⁺=431.14.

### General Method 2 for the Synthesis of Building Blocks B

### Methyl (R)-2-(2-amino-3-phenylpropoxy)-6-fluorobenzoate hydrochloride (B-2.1)

Step 1: Methyl (R)-2-(2-((tert-butoxycarbonyl)amino)-3-phenylpropoxy)-6-fluorobenzoate is prepared from methyl 2-fluoro-6-hydroxybenzoate and tert-butyl (R)-(1-hydroxy-3-phenylpropan-2-yl)carbamate in analogy to the procedure described for B-1.1 step 2. LC-MS J: t_{R}=2.28 min; [M-Boc+H]⁺=304.1.

Step 2: The title compound is prepared in analogy to the procedure described for B-1.1 step 3. LC-MS J: t_{R}=1.95 min; [M+H]⁺=304.1.

Listed in Table B-2 below are building blocks B that are prepared in analogy to the 2-step sequence described above for B-2.1. In cases where the HCl salt is highly hygroscopic, the amine is subjected to a basic workup to liberate its free base.

TABLE B-2

| | | | t_{R} [min] | MS-data |
|---|---|---|---|---|
| | | | LC-MS | m/z |
| No. | Starting Material | Product | Method | [M + H]⁺ |
| B-2.2 | Ethyl 2-chloro-6- | Ethyl (R)-2-(2-amino-3- | 2.05 | 334.1 |
| | hydroxybenzoate | phenylpropoxy)-6- | J | |
| | | chlorobenzoate hydrochloride | | |
| B-2.3 | Ethyl 4-hydroxy-2- | Ethyl (R)-4-(2-amino-3- | 2.00 | 321.1 |
| | methylthiazole-5-carboxylate | phenylpropoxy)-2- | | |
| | | methylthiazole-5-carboxylate | J | |
| | | hydrochloride | | |
| B-2.4 | Methyl 3-hydroxythiophene-2- | Methyl (R)-3-(2-amino-3- | 1.94 | 292.0 |
| | carboxylate | phenylpropoxy)thiophene-2- | J | |
| | | carboxylate hydrochloride | | |
| B-2.13 | Methyl 2-hydroxy-5- | Methyl (R)-2-(2-amino-3- | 0.73 | 316.31 |
| | methoxybenzoate | phenylpropoxy)-5- | B | |
| | | methoxybenzoate | | |
| | | hydrochloride | | |
| B-2.14 | Ethyl 2-hydroxybenzoate | Ethyl (R)-2-(2-amino-3- | 0.99 | 300.29 |
| | | phenylpropoxy)benzoate | I | |
| | | hydrochloride | | |

### Methyl (R)-6-(2-amino-3-phenylpropoxy)-2-methylbenzo[d]oxazole-7-carboxylate hydrochloride (B-2.5)

Step 1: Nitric acid (0.36 mL, 6.0 mmol) is carefully added to a 0° C. soln. of methyl 2,6-dihydroxybenzoate (1.0 g, 6.0 mmol) in acetic acid (10 mL) and the RM is warmed to RT and stirred for 1 h. The RM is poured into cold water and the precipitate collected by filtration and washed with additional cold water before being dried in vacuo to give methyl 2,6-dihydroxy-3-nitrobenzoate as a pink solid. LC-MS B: t_{R}=0.75 min; No ionisation. ¹H NMR (DMSO) δ: 11.73 (s, 1H), 10.94 (s, 1H), 8.05 (d, J=9.4 Hz, 1H), 6.60 (d, J=9.4 Hz, 1H), 3.81 (s, 3H).

Step 2: 4M HCl in dioxane (1.45 mL, 5.8 mmol) is added to a suspension of methyl 2,6-dihydroxy-3-nitrobenzoate (500 mg, 2.3 mmol) in triethyl ortho acetate (13.5 mL, 72 mmol) and the RM is evacuated/purged with N₂ (3×) before 10% Pd/C (173 mg, 7 mol %) is added. The RM is evacuated/purged with H₂ (3×) and stirred under a H₂ atm for 16 h. The RM is filtered through a pad of celite and the filtrate concentrated in vacuo to give methyl 6-hydroxy-2-methylbenzo[d]oxazole-7-carboxylate as a yellow solid. LC-MS B: t_{R}=0.78 min; [M+H]⁺=208.32. ¹H NMR (DMSO) δ: 10.69 (s, 1H), 7.76 (d, J=8.7 Hz, 1H), 6.96 (d, J=8.7 Hz, 1H), 3.97 (s, 3H), 2.60 (s, 3H).

Steps 3-4: The title compound is prepared from methyl 6-hydroxy-2-methylbenzo[d]oxazole-7-carboxylate following the sequence of reactions described for B-2.1. LC-MS B: t_{R}=0.69 min; [M+H]⁺=341.35. Note: Title compound is unstable and should not be stored for prolonged periods.

### Methyl (R)-6-(2-amino-3-phenylpropoxy)benzo[d]oxazole-7-carboxylate hydrochloride (B-2.6)

The title compound is prepared following the sequence of reactions described for B-2.5, substituting triethyl ortho acetate for trimethyl ortho formate in step 2. LC-MS B: t_{R}=0.66 min; [M+H]⁺=327.27. Note: Title compound is unstable and should not be stored for prolonged periods.

### Methyl (R)-6-(2-amino-3-phenylpropoxy)-1-methyl-1H-benzo[d]imidazole-7-carboxylate dihydrochloride (B-2.7)

Step 1: Methylamine (2 M in MeOH, 7.9 mL, 15.8 mmol) is added to a RT soln. of methyl 2,6-difluoro-3-nitrobenzoate (5.0 g, 22.6 mmol) in MeOH (40 mL) and the RM is stirred for 1 h. The RM is concentrated in vacuo and the residue suspended in a mix. of ⁱPrOH and water before being filtered. The filter cake is washed with water and dried before being purified by FC (eluting with 0% to 20% EtOAc in hept) to give methyl 6-fluoro-2-(methylamino)-3-nitrobenzoate as a yellow solid. LC-MS B: t_{R}=0.87 min; [M+H]⁺=229.38.

Step 2: NaH 60% Dispersion in mineral oil (549 mg, 13.7 mmol) is added portionwise to a 0° C. soln. of Boc-D-phenylalaninol (2.93 g, 11.4 mmol) in THF (60 mL) and after stirring for 10 min a soln. of methyl 6-fluoro-2-(methylamino)-3-nitrobenzoate (2.4 g, 10.4 mmol) in THF (10 mL) is added. The RM is warmed to RT and stirred for 1 h before being cooled back to 0° C. and quenched with water. The THF is evaporated in vacuo and the remaining aqueous phase is diluted with additional water and extracted with EtOAc (3×). The combined org. layers are washed with brine, dried over Na₂SO₄, filtered and evaporated in vacuo. The crude product is purified by FC (eluting with 0% to 30% EtOAc in hept) to give methyl (R)-6-(2-((tert-butoxycarbonyl)amino)-3-phenylpropoxy)-2-(methylamino)-3-nitrobenzoate as a yellow solid. LC-MS B: t_{R}=1.09 min; [M+H]⁺=460.26.

Steps 3-4: The title compound is prepared from methyl (R)-6-(2-((tert-butoxycarbonyl)amino)-3-phenylpropoxy)-2-(methylamino)-3-nitrobenzoate following the hydrogenation/cyclisation followed by Boc-cleavage sequence of reactions described for B-2.6. LC-MS B: t_{R}=0.54 min; [M+H]⁺=340.41.

### Methyl (R)-6-(2-amino-3-phenylpropoxy)-2-methylbenzo[d]oxazole-5-carboxylate hydrochloride (B-2.8)

The title compound is prepared from methyl 2,4-dihydroxybenzoate following the sequence of reactions described for B-2.5. LC-MS B: t_{R}=0.70 min; [M+H]⁺=341.39.

### Methyl (R)-6-(2-amino-3-phenylpropoxy)-1,2-dimethyl-1H-benzo[d]imidazole-7-carboxylate hydrochloride (B-2.9)

The title compound is prepared from methyl 2,6-difluoro-3-nitrobenzoate following the sequence of reactions described for B-2.7, using triethyl ortho acetate instead of trimethyl ortho formate. LC-MS B: t_{R}=0.50 min; [M+H]⁺=354.45.

### Ethyl (R)-2-(2-amino-3-phenylpropoxy)imidazo[1,2-a]pyridine-3-carboxylate dihydrochloride (B-2.10)

Step 1: 2-Aminopyridine (7.08 g, 75 mmol) is suspended in diethyl bromomalonate (38.5 mL, 226 mmol) and heated to 100° C. for 1.5 h. The RM is partitioned between water and EtOAc and the layers are separated. The org. phase is discarded and the aq. phase is freeze dried to give the crude product that is purified by prep. HPLC (basic) to give ethyl 2-hydroxyimidazo[1,2-a]pyridine-3-carboxylate as a cream solid. LC-MS J: t_{R}=0.41 min; [M+H]⁺=207.1.

Steps 2-3: The title compound is prepared from ethyl 2-hydroxyimidazo[1,2-a]pyridine-3-carboxylate in analogy to the procedure described for B-2.1. LC-MS I: t_{R}=0.91 min; [M+H]⁺=340.28.

### Methyl (R)-5-(2-amino-3-phenylpropoxy)-2,3-dihydrobenzofuran-4-carboxylate hydrochloride (B-2.11)

Steps 1-2: A suspension of methyl 2,5-dihydroxybenzoate (1.02 g, 6.1 mmol) and MgSO₄ (2.1 g, 17.5 mmol) in Et₂O (10 mL) is purged with argon for 10 min before Ag₂O (3.46 g, 14.9 mmol) is added and the RM is stirred for 16 h. The RM is filtered over a pad of celite and the filtrate is concentrated in vacuo to give the crude product methyl 3,6-dioxocyclohexa-1,4-diene-1-carboxylate that is re-dissolved in PhMe (50 mL) before n-butyl vinyl ether (1.7 mL, 13.1 mmol) is added and the RM is heated to 45° C. for 19 h. The RM is poured into water and the phases are separated. The aq. phase is extracted with EtOAc (3×) and the combined org. phases are washed with brine, dried over Na₂SO₄, filtered and evaporated in vacuo. The crude product is purified by FC (eluting with 1% to 50% EtOAc in hept) to give methyl 2-butoxy-5-hydroxy-2,3-dihydrobenzofuran-4-carboxylate as a white solid. LC-MS J: t_{R}=2.23 min; [M+H]⁺=267.1.

Step 3: TFA (0.9 mL, 11.75 mmol) is added to a soln. of methyl 2-butoxy-5-hydroxy-2,3-dihydrobenzofuran-4-carboxylate (761 mg, 2.86 mmol) in PhMe (25 mL) and the RM is heated to reflux for 4.5 h. The RM is poured into water and the phases are separated. The aq. phase is extracted with EtOAc (3×) and the combined org. phases are washed with brine, dried over Na₂SO₄, filtered and evaporated in vacuo. The crude product is purified by FC (eluting with 0% to 50% EtOAc in hept) to give methyl 5-hydroxybenzofuran-4-carboxylate as a white solid. LC-MS J: t_{R}=2.00 min; No ionisation. ¹H NMR (DMSO) δ: 10.83 (bs, 1H), 8.12 (d, J=2.2 Hz, 1H), 7.82 (d, J=8.9 Hz, 1H), 7.19 (d, J=2.1 Hz, 1H), 6.95 (d, J=8.9 Hz, 1H), 4.00 (s, 3H).

Step 4: A soln. of methyl 5-hydroxybenzofuran-4-carboxylate (395 mg, 2.06 mmol) and AcOH (1.3 mL, 22.5 mmol) in EtOAc (20 mL) is evacuated/purged with N₂ (3×) before 10% Pd/C (109 mg, 5 mol %) is added. The RM is evacuated/purged with H₂ (3×) and stirred under a H₂ atm for 21 h. The RM is filtered through a pad of celite and the filtrate concentrated in vacuo to give methyl 6-hydroxy-2-methylbenzo[d]oxazole-7-carboxylate as a white solid. LC-MS F: t_{R}=1.88 min; [M+H]⁺=195.0.

Steps 5-6: The title compound is prepared from methyl 5-hydroxy-2,3-dihydrobenzofuran-4-carboxylate in analogy to the procedure described for B-2.1. LC-MS J: t_{R}=1.97 min; [M+H]⁺=328.1.

### Methyl (R)-3-(2-amino-3-phenylpropoxy)-6-methoxypicolinate hydrochloride (B-2.12)

Step 1: Cs₂CO₃ (2.4 g, 7.4 mmol) and BnBr (1.31 mL, 11.1 mmol) are added to a RT soln. of methyl 3-hydroxypicolinate (0.94 g, 6.1 mmol) in DMF (20 mL) and the RM is heated to 70° C. for 2 h. The RM is concentrated in vacuo and the residue is partitioned between water and EtOAc and extracted. The layers are separated, and the aq. phase is re-extracted with EtOAc (2×). The combined org. extracts are washed with brine, dried over Na₂SO₄, filtered and evaporated in vacuo. The crude product is purified by FC (eluting with 30% to 45% EtOAc in hept) to give methyl 3-(benzyloxy)picolinate as a brown oil. LC-MS J: t_{R}=1.86 min; [M+H]⁺=244.0.

Step 2: mCPBA (1.51 g, 6.1 mmol) is added to a 0° C. soln. of methyl 3-(benzyloxy)picolinate (1.19 g, 4.9 mmol) in DCM (10 mL) and the RM is warmed to RT and stirred for 16 h. The RM is partitioned between sat. aq. NaHSO₃ and DCM and extracted. The layers are separated, and the aq. phase is re-extracted with DCM (2×). The combined org. extracts are washed with sat. aq. NaHCO₃, dried over Na₂SO₄, filtered and evaporated in vacuo to give 3-(benzyloxy)-2-(methoxycarbonyl)pyridine 1-oxide as a brown solid. LC-MS J: t_{R}=1.72 min; [M+H]⁺=260.0.

Step 3: Ac₂O (7.0 mL, 74.2 mmol) is added to 3-(benzyloxy)-2-(methoxycarbonyl)pyridine 1-oxide (1.27 g, 4.9 mmol) and the RM is heated to 100° C. for 1 h. Additional Ac₂O (7 mL, 74.2 mmol) is added and heating continued for 2 h. EtOH (10 mL) is added and the RM is heated to reflux for 1 h before being cooled to RT overnight. The RM is concentrated in vacuo and the residue azeotroped with PhMe (1×) before 2M NaOH in MeOH (10 mL) is added and the RM is heated to 80° C. for 3 h. The MeOH is evaporated in vacuo and the remaining aq. is acidified with 1M HCl before being extracted with DCM (4×). The combined org. extracts are washed with brine, dried over Na₂SO₄, filtered and evaporated in vacuo to give 3-(benzyloxy)-6-hydroxypicolinic acid as a brown solid. LC-MS G: t_{R}=2.74 min; [M+H]⁺=246.0.

Step 4: Ag₂CO₃ (4.5 g, 16.3 mmol) and Mel (0.56 mL, 9.0 mmol) are added to a suspension of 3-(benzyloxy)-6-hydroxypicolinic acid (1.0 g, 4.1 mmol) in acetone (60 mL) and the RM is heated to reflux for 2 h. The RM is cooled to RT and acidified with 1 M HCl before being concentrated in vacuo. The residue is partitioned between water and DCM and extracted. The layers are filtered and separated, and the aq. phase is re-extracted with DCM (2×). The combined org. extracts are washed with brine, dried over Na₂SO₄, filtered and evaporated in vacuo. The crude product is purified by FC (eluting with 10% to 70% EtOAc in hept) to give methyl 3-(benzyloxy)-6-methoxypicolinate as a colourless oil. LC-MS J: t_{R}=1.98 min; [M+H]⁺=274.2.

Step 5: A soln. of methyl 3-(benzyloxy)-6-methoxypicolinate (847 mg, 2.94 mmol) in EtOH (20 mL) is evacuated/purged with N₂ (3×) before 10% Pd/C (157 mg, 5 mol %) is added. The RM is evacuated/purged with H₂ (3×) and stirred under a H₂ atm for 2 h. The RM is filtered through a pad of celite and the filtrate concentrated in vacuo to give methyl 3-hydroxy-6-methoxypicolinate as a white solid. LC-MS J: t_{R}=1.48 min; No ionisation.

Steps 6-7: The title compound is prepared from methyl 3-hydroxy-6-methoxypicolinate in analogy to the procedure described for B-2.1. LC-MS J: t_{R}=1.85 min; [M+H]⁺=317.2.

### Methyl (R)-5-(2-amino-3-phenylpropoxy)-2-methylbenzo[d]oxazole-4-carboxylate hydrochloride (B-2.15)

The title compound is prepared from methyl 2,5-dihydroxybenzoate following the sequence of reactions described for B-2.5. LC-MS D: t_{R}=0.63 min; [M+H]⁺=341.37.

### Methyl (R)-3-(2-amino-3-phenylpropoxy)isoquinoline-4-carboxylate dihydrochloride (B-2.16)

Step 1: NBS (1.30 g, 7.3 mmol) is added portionwise to a 0° C. soln. of isoquinolin-3-amine (1.0 g, 6.9 mmol) in DCM (20 mL) and EtOH (10 mL) and the RM is stirred for 30 min before being warmed to RT overnight. The RM is concentrated in vacuo and the residue is triturated with DCM and filtered. The filtrate is concentrated in vacuo and the residue purified by FC (eluting with 25% to 40% EtOAc in hept) to give 4-bromoisoquinolin-3-amine as a brown solid. Subsequent trituration with ⁱPr₂O further enhances its purity. LC-MS J: t_{R}=1.84 min; [M+H]⁺=223.0.

Step 2: A soln. of 4-bromoisoquinolin-3-amine (796 mg, 3.6 mmol) and DIPEA (1.87 mL, 10.7 mmol) in DMF (10 mL) and MeOH (5 mL) is evacuated/purged with CO (3×) before Pd(dppf)Cl₂ (261 mg, 0.36 mmol) is added. The RM is evacuated/purged with CO (3×) and stirred under a CO atm at 75° C. for 20 h. The RM is cooled to RT and concentrated in vacuo and the residue is partitioned between water and EtOAc and extracted. The layers are separated, and the aq. phase is re-extracted with EtOAc (2×). The combined org. extracts are washed with brine, dried over Na₂SO₄, filtered and evaporated in vacuo to give methyl 3-aminoisoquinoline-4-carboxylate as a yellow solid. LC-MS J: t_{R}=1.78 min; [M+H]⁺=203.1.

Step 3: A soln. of NaNO₂ (135 mg, 2.0 mmol) in H₂O (0.6 mL) is added to a 0° C. suspension of methyl 3-aminoisoquinoline-4-carboxylate (330 mg, 1.63 mmol) in 2.5M aq. H₂SO₄ (4 mL, 10 mmol) and the RM is stirred for 1.5 h. The RM is neutralised by the addition of 2M aq. NaOH and extracted with EtOAc (4×). The combined org. extracts are washed with brine, dried over Na₂SO₄, filtered and evaporated in vacuo to give methyl 3-hydroxyisoquinoline-4-carboxylate as a yellow solid. LC-MS F: t_{R}=1.52 min; [M+H]⁺=204.0.

Steps 4-5: The title compound is prepared from methyl 3-hydroxyisoquinoline-4-carboxylate following the sequence of reactions described for B-2.1. LC-MS J: t_{R}=1.97 min; [M+H]⁺=337.2.

### Methyl (R)-6-(2-amino-3-phenylpropoxy)imidazo[1,2-a]pyridine-5-carboxylate dihydrochloride (B-2.17)

Step 1: Br₂ (0.81 mL, 15.7 mmol) is added dropwise to a 0° C. soln. of methyl-3-hydroxypicolinate (2.41 g, 15.7 mmol) in water (110 mL) and the RM is warmed to RT and stirred overnight. The RM is quenched with 40% aq. sodium bisulfite soln. and extracted with DCM (2×). The combined org. extracts are washed with brine, dried over Na₂SO₄, filtered and evaporated in vacuo to give methyl 6-bromo-3-hydroxypicolinate as a white solid. LC-MS G: t_{R}=3.19 min; [M+H]⁺=231.9.

Step 2: Methyl (R)-6-bromo-3-(2-((tert-butoxycarbonyl)amino)-3-phenylpropoxy)picolinate is prepared from methyl 6-bromo-3-hydroxypicolinate and tert-butyl (R)-(1-hydroxy-3-phenylpropan-2-yl)carbamate in analogy to the procedure described for B-1.1 step 2. LC-MS F: t_{R}=2.16 min; [M+H-^{t}Bu]⁺=409.0.

Step 3: Pd₂(dba)₃ (483 mg, 0.53 mmol) and XPhos (201 mg, 0.42 mmol) are added to a RT mix. of methyl (R)-6-bromo-3-(2-((tert-butoxycarbonyl)amino)-3-phenylpropoxy)picolinate (5.0 g, 10.5 mmol), benzyl carbamate (1.67 g, 11.1 mmol) and Cs₂CO₃ (5.15 g, 15.8 mmol) in dioxane (130 mL) and the RM is heated to 95° C. and stirred for 48 h. The RM is cooled to RT, filtered and the filtrate is concentrated in vacuo before being purified by FC (eluting with 0% to 40% EtOAc in hept) to give methyl (R)-6-(((benzyloxy)carbonyl)amino)-3-(2-((tert-butoxycarbonyl)amino)-3-phenylpropoxy)picolinate as an orange solid. LC-MS F: t_{R}=2.24 min; [M+H]⁺=536.2.

Step 4: A soln. of methyl (R)-6-(((benzyloxy)carbonyl)amino)-3-(2-((tert-butoxycarbonyl)amino)-3-phenylpropoxy)picolinate (1.43 g, 2.19 mmol) in EtOH (20 mL) is purged with N₂/vacuum (3×) before 10% Pd/C (70 mg, 0.07 mmol) is added. After inertising another three times a H₂ balloon is connected and the RM is stirred at 55° C. for 1 h. The mix. is filtered over a celite plug rinsing with EtOH. The filtrate is concentrated to give methyl (R)-6-amino-3-(2-((tert-butoxycarbonyl)amino)-3-phenylpropoxy)picolinate as a yellow oil. LC-MS J: t_{R}=2.00 min; [M+H]⁺=402.2.

Step 5: 50% aq. 2-Chloroacetaldehyde (0.475 mL, 3.74 mmol) is added to a mix. of methyl (R)-6-amino-3-(2-((tert-butoxycarbonyl)amino)-3-phenylpropoxy)picolinate (0.50 g, 1.25 mmol) and NaHCO₃ (209 mg, 2.49 mmol) in EtOH (15 mL) and the RM is heated to 70° C. and stirred for 5 h. The RM is concentrated in vacuo and the residue is partitioned between water and EtOAc and extracted. The layers are separated, and the aq. phase is re-extracted with EtOAc (2×). The combined org. extracts are washed with brine, dried over Na₂SO₄, filtered and evaporated in vacuo. The crude product is purified by FC (eluting with 50% to 100% EtOAc in hept) to give methyl (R)-6-(2-((tert-butoxycarbonyl)amino)-3-phenylpropoxy)imidazo[1,2-a]pyridine-5-carboxylate as a white solid. LC-MS I: t_{R}=1.01 min; [M+H]⁺=426.52.

Step 6: The title compound is prepared in analogy to the procedure described for B-1.1 step 3. LC-MS J: t_{R}=1.83 min; [M+H]⁺=326.2.

### General Method 3 for the Synthesis of Building Blocks B

### Ethyl (R)-4-(2-amino-3-phenylpropoxy)nicotinate dihydrochloride (B-3.1)

Step 1: H₂SO₄ (1.86 mL, 34.9 mmol) is added dropwise to a 0° C. soln. of 4-hydroxynicotinic acid (5.0 g, 34.9 mmol) in EtOH (50 mL) and the RM is heated to reflux for 3 d. After concentration in vacuo, sat. aq. NaHCO₃ is carefully added to the residue and the solid filtered off and dried to give ethyl 4-hydroxynicotinate as an off-white solid. LC-MS D: t_{R}=0.37 min; [M+H]⁺=168.05.

Step 2: Ethyl (R)-4-(2-((tert-butoxycarbonyl)amino)-3-phenylpropoxy)nicotinate is prepared from ethyl 4-hydroxynicotinate and tert-butyl (R)-(1-hydroxy-3-phenylpropan-2-yl)carbamate in analogy to the procedure described for B-1.1 step 2. LC-MS J: t_{R}=2.17 min; [M+H]⁺=401.2.

Step 3: The title compound is prepared in analogy to the procedure described for B-1.1 step 3. LC-MS B: t_{R}=0.52 min; [M+H]⁺=301.28.

Listed in Table B-3 below are building blocks B that are prepared in analogy to the 3-step sequence described above for B-3.1. In cases where the HCl salt is highly hygroscopic, the amine is subjected to a basic workup to liberate its free base.

TABLE B-3

| | | | t_{R} [min] | MS-data |
|---|---|---|---|---|
| | | | LC-MS | m/z |
| No. | Starting Material | Product | Method | [M + H]⁺ |
| B-3.2 | 3-Hydroxyisonicotinic acid | Ethyl (R)-3-(2-amino-3- | 1.88 | 301.1 |
| | | phenylpropoxy)isonicotinate | J | |
| | | dihydrochloride | | |

### General Method 4 for the Synthesis of Building Blocks B

### Benzyl (R)-2-(2-amino-3-phenylpropoxy)-6-(trifluoromethoxy)benzoate hydrochloride (B-4.1)

Step 1: A soln. of 3-(trifluoromethoxy)phenol (5.0 g, 28.1 mmol) in THF (40 mL) is added dropwise to a RT suspension of NaH (1.35 g, 33.7 mmol) in THF (50 mL) and the resulting mix. is stirred for 15 min before methoxymethyl bromide (2.98 mL, 36.5 mmol) is added dropwise. After stirring for 1 h the RM is quenched by the addition of sat. aq. Na₂CO₃, diluted with some water, and extracted with ⁱPr₂O. The org. phase is washed with NaHCO₃, brine, dried over Na₂SO₄, filtered and evaporated in vacuo to give 1-(methoxymethoxy)-3-(trifluoromethoxy)benzene as a colourless oil. LC-MS J: t_{R}=2.13 min; No ionisation.

Step 2: A soln. of 1-(methoxymethoxy)-3-(trifluoromethoxy)benzene (3.0 g, 13.5 mmol) in THF (7 mL) is added dropwise to a −78° C. soln. of sBuLi (1.4 M in cyclohexane, 12.54 mL, 17.55 mmol) in a mix. of THF (10 mL) and cyclohexane (15 mL) and the RM is stirred for 1.5 h. The RM is quenched onto freshly ground dry ice and then warmed to RT. After stirring for 15 min, a few drops of MeOH are added before the RM is concentrated in vacuo. The intermediate lithium carboxylate is dissolved in DMF (20 mL) before KHCO₃ (0.41 g, 4.1 mmol) and benzyl bromide (1.93 mL, 16.2 mmol) are added and the RM is stirred for 18 h. The RM is filtered and the filtrate concentrated in vacuo. The residue is partitioned between water and EtOAc and extracted. The layers are separated, and the aq. phase is re-extracted with EtOAc (2×). The combined org. extracts are washed with brine, dried over Na₂SO₄, filtered and evaporated in vacuo. The crude product is purified by FC (eluting with 0% to 15% EtOAc in hept) to give benzyl 2-(methoxymethoxy)-6-(trifluoromethoxy)benzoate as a colourless oil. LC-MS J: t_{R}=2.24 min; [M+H]⁺=357.1.

Step 3: TFA (2 mL, 26.1 mmol) is added to a soln. of benzyl 2-(methoxymethoxy)-6-(trifluoromethoxy)benzoate (1.39 g, 3.9 mmol) in DCM (20 mL) and the resulting mix. is stirred for 1 h. The RM is concentrated in vacuo and the residue is co-evaporated with DCM (2×) to give benzyl 2-hydroxy-6-(trifluoromethoxy)benzoate as a white solid. LC-MS J: t_{R}=2.22 min; [M−H]=311.0.

Step 4: Benzyl (R)-2-(2-((tert-butoxycarbonyl)amino)-3-phenylpropoxy)-6-(trifluoromethoxy)benzoate is prepared from benzyl 2-hydroxy-6-(trifluoromethoxy)benzoate and tert-butyl (R)-(1-hydroxy-3-phenylpropan-2-yl)carbamate in analogy to the procedure described for B-1.1 Step 2. LC-MS J: t_{R}=2.50 min; [M-Boc+H]⁺=446.1.

Step 5: The title compound is prepared in analogy to the procedure described for B-1.1 Step 3. LC-MS J: t_{R}=2.30 min; [M+H]⁺=446.1.

Listed in Table B-4 below are building blocks B that are prepared in analogy to the 5-step sequence described above for B-4.1. In cases where the HCl salt is highly hygroscopic, the amine is subjected to a basic workup to liberate its free base.

TABLE B-4

| | | | t_{R} [min] | MS-data |
|---|---|---|---|---|
| | | | LC-MS | m/z |
| No. | Starting Material | Product | Method | [M + H]⁺ |
| B-4.2 | Benzo[d][1,3]dioxol-5-ol | Benzyl (R)-5-(2-amino-3- | 2.09 | 406.1 |
| | | phenylpropoxy)benzo[d][1,3]dioxole- | J | |
| | | 4-carboxylate hydrochloride | | |
| B-4.3 | Benzo[d][1,3]dioxol-2,2-d2- | Benzyl (R)-5-(2-amino-3- | 2.16 | 408.2 |
| | 5-ol | phenylpropoxy)benzo[d][1,3]dioxole- | J | |
| | | 4-carboxylate-2,2-d2 hydrochloride | | |
| B-4.4 | 2,2- | Benzyl (R)-5-(2-amino-3- | 2.31 | 442.1 |
| | Difluorobenzo[d][1,3]dioxol- | phenylpropoxy)-2,2- | J | |
| | 5-ol | difluorobenzo[d][1,3]dioxole-4- | | |
| | | carboxylate hydrochloride | | |

### Ethyl (R)-4-(2-amino-3-phenylpropoxy)-2-methylnicotinate dihydrochloride (B-4.5)

Step 1: NaH (370 mg, 9.26 mmol) is added to a 0° C. soln. of 2-chloro-4-hydroxypyridine (1.0 g, 7.72 mmol) in DMF (20 mL) and the RM is warmed to RT and stirred for 15 min before being cooled back to 0° C. Chloromethyl methyl ether (1.17 mL, 15.4 mmol) is added and the RM is warmed to RT and stirred for 1 h. The RM is quenched by the addition of sat. aq. Na₂CO₃, diluted with some water, and extracted with Et₂O. The org. phase is washed with NaHCO₃, brine, dried over Na₂SO₄, filtered and evaporated in vacuo to give the crude product that is purified by FC (eluting with 20% to 100% EtOAc in hept) to give 2-chloro-4-(methoxymethoxy)pyridine as a colourless oil. LC-MS I: t_{R}=0.72 min; [M+H]⁺=174.09.

Step 2: nBuLi (2.5 M in hex, 1.18 mL, 2.94 mmol) is added to a −78° C. soln. of 2-chloro-4-(methoxymethoxy)pyridine (413 mg, 2.36 mmol) in THF (7 mL) and after stirring for 30 min the RM is added via cannula to a −78° C. soln. of ethyl chloroformate (0.23 mL, 2.36 mmol) in THF (4 mL) and stirred for 1h at −78° C. The RM is warmed to RT and stirred for 2h before being quenched with NaHCO₃ and extracted with EtOAc (3×). The combined org. extracts are washed with brine, dried over Na₂SO₄, filtered and evaporated in vacuo. The crude product is purified by FC (eluting with 10% to 70% EtOAc in hept) to give ethyl 2-chloro-4-(methoxymethoxy)nicotinate as a yellow oil. LC-MS B: t_{R}=0.82 min; [M+H]⁺=246.29.

Step 3: ZnMe₂ (2 M in PhMe, 0.46 mL, 0.93 mmol) is added dropwise to a RT soln. of ethyl 2-chloro-4-(methoxymethoxy)nicotinate (120 mg, 0.46 mmol), and Pd(dppf)Cl₂.DCM (3.8 mg, 0.005 mmol) in dioxane (2 mL) and the RM is heated to 90° C. and stirred for 1 h. The RM is concentrated in vacuo and the residue is partitioned between water and EtOAc and extracted. The layers are separated, and the aq. phase is re-extracted with EtOAc (2×). The combined org. extracts are washed with brine, dried over Na₂SO₄, filtered and evaporated in vacuo. The crude product is purified by prep. HPLC (basic) to give ethyl 4-(methoxymethoxy)-2-methylnicotinate as a colourless oil. LC-MS I: t_{R}=0.71 min; [M+H]⁺=226.18.

Step 4: 4M HCl in dioxane (0.55 mL) is added to a RT soln. of ethyl 4-(methoxymethoxy)-2-methylnicotinate (50 mg, 0.22 mmol) in dioxane (1 mL) and the RM is stirred for 16 h. The volatiles are removed in vacuo and the residue is suspended in Et₂O and concentrated to give ethyl 4-hydroxy-2-methylnicotinate hydrochloride as a white solid. LC-MS I: t_{R}=0.30 min; [M+H]⁺=182.23.

Step 5: Ethyl (R)-4-(2-((tert-butoxycarbonyl)amino)-3-phenylpropoxy)-2-methylnicotinate is prepared from ethyl 4-hydroxy-2-methylnicotinate hydrochloride and tert-butyl (R)-(1-hydroxy-3-phenylpropan-2-yl)carbamate in analogy to the procedure described for B-1.1 Step 2. LC-MS B: t_{R}=0.82 min; [M+H]⁺=415.41.

Step 6: The title compound is prepared in analogy to the procedure described for B-1.1 Step 3. LC-MS B: t_{R}=0.52 min; [M+H]⁺=315.32.

### Benzyl (R)-6-(2-amino-3-phenylpropoxy)-2,3-dihydrobenzofuran-7-carboxylate hydrochloride (B-4.6)

Step 1: Benzyl bromide (8.9 mL, 74.6 mmol) and K₂CO₃ (14.7 g, 107 mmol) are added to a RT soln. of 6-hydroxybenzofuran-3 (2H)-one (8.0 g, 53.3 mmol) in DMF (80 mL) and the RM is stirred for 2 h. The RM is poured into cold water and the precipitate is collected by filtration and dried at 40° C. in a vacuum oven for 48 h. 6-(benzyloxy)benzofuran-3 (2H)-one is isolated as an orange solid. LC-MS J: t_{R}=2.03 min; [M+H]⁺=241.0.

Step 2: 6-(benzyloxy)Benzofuran-3 (2H)-one (11.25 g, 46.8 mmol) is added portionwise to a 0° C. soln. of AlCl₃ (6.87 g, 51.5 mmol) and LiAlH₄ (19.5 mL, 46.8 mmol, 2.4M in THF) in THF (200 mL) and the RM is warmed to RT and stirred for 2 h. The RM is cooled to 0° C. and quenched with 0.5M aq. NaOH (400 mL) and extracted with EtOAc (3×). The combined org. extracts are washed with brine, dried over Na₂SO₄, filtered and evaporated in vacuo to give 6-(benzyloxy)benzofuran as an orange oil. GC-MS MC: t_{R}=4.74 min; [M]⁺=224.1. Note: Contains 6-(benzyloxy)-2,3-dihydrobenzofuran as a minor side product.

Step 3: 2,3-Dihydrobenzofuran-6-ol is prepared from 6-(benzyloxy)benzofuran following the procedure described for B-2.11 step 4. GC-MS MC: t_{R}=3.38 min; [M]⁺=136.1.

Steps 4-8: The title compound is prepared from 2,3-dihydrobenzofuran-6-ol in analogy to the procedure described for B-4.1. LC-MS J: t_{R}=2.30 min; [M+H]⁺=446.1.

### Benzyl (R)-6-(2-amino-3-phenylpropoxy)quinoline-5-carboxylate dihydrochloride (B-4.7)

Step 1: A soln. of Br₂ (0.37 mL, 7.2 mmol) in AcOH (5 mL) is added to a RT soln. of quinolin-6-ol (1.0 g, 6.9 mmol) and NaOAc (0.62 g, 7.6 mmol) in AcOH (15 mL) and the RM is stirred for 30 min. The RM is quenched with sat. aq. NaHSO₃ and neutralised with 2M aq. NaOH and Na₂CO₃ before being extracted with EtOAc (2×). The combined org. extracts are washed with brine and concentrated in vacuo. The residue is taken up in PhMe and concentrated in vacuo (2×) to give 5-bromoquinolin-6-ol as a brown solid. LC-MS J: t_{R}=1.16 min; [M+H]⁺=221.9.

Step 2: 5-Bromo-6-(methoxymethoxy)quinoline is prepared from 5-bromoquinolin-6-ol in analogy to the procedure described for B-4.1 step 1. LC-MS J: t_{R}=2.30 min; [M+H]⁺=446.1.

Step 3: nBuLi (1.6 M in hex, 5.7 mL, 9.1 mmol) is added dropwise to a −78° C. soln. of 5-bromo-6-(methoxymethoxy)quinoline (2.45 g, 9.1 mmol) in THF (50 mL) and the RM is stirred for 30 min. The RM is quenched with freshly ground dry ice (12 g, 273 mmol) and then warmed to RT and stirred for 30 min. The RM is concentrated in vacuo and the intermediate lithium carboxylate is dissolved in DMF (30 mL) before benzyl bromide (1.3 mL, 11 mmol) is added and the RM is heated to 60° C. for 10 min. The RM is cooled to RT and partitioned between sat. aq.

NaHCO₃ and EtOAc and extracted. The layers are separated, and the aq. phase is re-extracted with EtOAc (2×). The combined org. extracts are washed with brine, dried over Na₂SO₄, filtered and evaporated in vacuo. The crude product is purified by FC (eluting with 30% to 100% EtOAc in hept) to give benzyl 6-(methoxymethoxy)quinoline-5-carboxylate as a yellow oil. LC-MS J: t_{R}=2.02 min; [M+H]⁺=324.1.

Steps 4-6: The title compound is prepared from benzyl 6-(methoxymethoxy)quinoline-5-carboxylate in analogy to the procedure described for B-4.1 steps 3-5. LC-MS B: t_{R}=0.77 min; [M+H]⁺=413.16.

### Benzyl (R)-7-(2-amino-3-phenylpropoxy)quinoline-8-carboxylate dihydrochloride (B-4.8)

Step 1: DCM (40 mL) is added to a soln. of quinolin-7-ol (10 g, 68.9 mmol) in AcOH (20 mL) and the resulting suspension is cooled to 0° C. before a soln. of Br₂ (3.87 mL, 75 mmol) in AcOH (20 mL) is added slowly and the RM is stirred for 2 h. The suspension is diluted with EtOAc and filtered and the filter residue is washed with EtOAc and Et₂O and dried in vacuo at 40° C. to give 8-bromoquinolin-7-ol hydrobromide as a brown solid. LC-MS J: t_{R}=0.27 min; [M+H]⁺=225.9.

Steps 2-6: The title compound is prepared from 8-bromoquinolin-7-ol hydrobromide in analogy to the procedure described for B-4.7 steps 2-6. LC-MS J: t_{R}=2.07 min; [M+H]⁺=413.1.

### Benzyl (R)-6-(2-amino-3-phenylpropoxy)-3-fluoroquinoline-5-carboxylate dihydrochloride (B-4.9)

The title compound is prepared from 3-fluoroquinolin-6-ol in analogy to the procedure described for B-4.7. LC-MS J: t_{R}=2.16 min; [M+H]⁺=431.2.

### Benzyl (R)-2-(2-amino-3-phenylpropoxy)-4-methoxynicotinate hydrochloride (B-4.10)

Step 1: Benzyl 2-fluoro-4-methoxynicotinate is prepared from 2-fluoro-4-methoxypyridine in analogy to the procedure described for B-4.7 step-3. LC-MS F: t_{R}=1.96 min; [M+H]⁺=262.0.

Step 2: A soln. of KO^{t}Bu (258 mg, 2.3 mmol) in THF (3.5 mL) is added to a 0° C. soln. of benzyl 2-fluoro-4-methoxynicotinate (600 mg, 2.3 mmol) and tert-butyl (R)-(1-hydroxy-3-phenylpropan-2-yl)carbamate (577 mg, 2.3 mmol) in THF (20 mL) and the RM is warmed to RT and stirred for 30 min. The RM is concentrated in vacuo and residue is purified by FC (eluting with 10% to 45% EtOAc in hept) to give benzyl (R)-2-(2-((tert-butoxycarbonyl)amino)-3-phenylpropoxy)-4-methoxynicotinate as a colourless oil. LC-MS F: t_{R}=2.30 min; [M+H]⁺=493.1.

Step 3: The title compound is prepared from benzyl (R)-2-(2-((tert-butoxycarbonyl)amino)-3-phenylpropoxy)-4-methoxynicotinate in analogy to the procedure described for B-1.1 step 3. LC-MS F: t_{R}=1.76 min; [M+H]⁺=393.1.

### Benzyl (R)-5-(2-amino-3-phenylpropoxy)-2-methoxyisonicotinate trifluoroacetate (B-4.11)

Step 1: 2-Methoxy-5-(methoxymethoxy)pyridine is prepared from 6-methoxypyridin-3-ol in analogy to the procedure described for B-4.1 step 1. LC-MS J: t_{R}=1.67 min; [M+H]⁺=170.1.

Steps 2-5: The title compound is prepared from 2-methoxy-5-(methoxymethoxy)pyridine in analogy to the procedure described for B-4.7 steps 3-6 substituting HCl for TFA in the Boc cleavage step. LC-MS J: t_{R}=2.16 min; [M+H]⁺=393.1.

### Ethyl (R)-4-(2-amino-3-phenylpropoxy)-2-methoxy-6-methylnicotinate (B-4.12)

Step 1: 4-(Benzyloxy)-6-methylpyridin-2-ol is prepared from 6-methylpyridine-2,4-diol following the procedure described for B-4.6 step 1. LC-MS I: t_{R}=0.71 min; [2M+H]⁺=431.31.

Step 2: Mel (0.438 mL, 6.97 mmol) is added to a RT mix. of 4-(benzyloxy)-6-methylpyridin-2-ol (500 mg, 2.32 mmol) and Ag₂CO₃ (1.29 g, 4.65 mmol) in DCM (20 mL) and the RM is irradiated in a MW oven at 100° C. for 1 h. The RM is filtered and the solids washed with DCM before the filtrate is concentrated in vacuo and the residue is purified by FC (eluting with 20% to 100% EtOAc in hept) to give 4-(benzyloxy)-2-methoxy-6-methylpyridine as a colourless oil. LC-MS I: t_{R}=1.07 min; [M+H]⁺=230.25.

Step 3: nBuLi (2.5 M in hex, 3.63 mL, 9.1 mmol) is added dropwise to a −78° C. soln. of 4-(benzyloxy)-2-methoxy-6-methylpyridine (1.66 g, 7.3 mmol) in THF (25 mL) and the RM is stirred for 30 min before ethyl chloroformate (0.70 mL, 7.3 mmol) is added dropwise. The RM is warmed to RT and quenched by addition of sat. aq. NaHCO₃ and extracted with EtOAc. The layers are separated and the aq. phase is re-extracted with EtOAc (2×) and the combined org. layers are washed with brine, dried over Na₂SO₄, filtered and evaporated in vacuo. The crude product is purified by FC (eluting with 20% EtOAc in hept) to give ethyl 4-(benzyloxy)-2-methoxy-6-methylnicotinate as a colourless oil. LC-MS I: t_{R}=1.11 min; [M+H]⁺=302.29.

Steps 4-6: The title compound is prepared from ethyl 4-(benzyloxy)-2-methoxy-6-methylnicotinate in analogy to the procedure described for B-2.12 steps 5-7. Note: Boc-cleavage is performed using TFA instead of HCl and the title compound is isolated as its free base after a basic workup. LC-MS I: t_{R}=0.97 min; [M+H]⁺=345.32.

### Ethyl (R)-4-(2-amino-3-phenylpropoxy)-2-(methoxy-d3)-6-methylnicotinate (B-4.13)

The title compound is prepared in analogy to the procedure described for B-4.12, substituting Mel by CD₃I in step 2. LC-MS I: t_{R}=0.97 min; [M+H]⁺=348.35.

### Benzyl (R)-6-(2-amino-3-phenylpropoxy)-8-methylquinoline-5-carboxylate (B-4.14)

Step 1: Meldrum's acid (6.04 g, 41.1 mmol) and triethyl orthoformate (6.06 mL, 35.7 mmol) are added to a RT soln. of 4-methoxy-2-methylaniline (5.0 g, 35.7 mmol) in EtOH (50 mL) and the RM is heated to 80° C. for 2h. The RM is cooled to RT and the precipitate is collected by filtration washing with EtOH and dried under HV to give 5-(((4-methoxy-2-methylphenyl)amino)methylene)-2,2-dimethyl-1,3-dioxane-4,6-dione as a white solid. LC-MS B: t_{R}=0.90 min; [M+H]⁺=292.13.

Step 2: 5-(((4-Methoxy-2-methylphenyl)amino)methylene)-2,2-dimethyl-1,3-dioxane-4,6-dione (8.19 g, 28.1 mmol) is dissolved in Dowtherm A (50 mL) and heated to 250° C. for 5 min. The RM is cooled to RT and diluted with Et₂O and the precipitate is collected by filtration and washed with Et₂O before being dried under HV to give 6-methoxy-8-methylquinolin-4-ol as a brown solid. LC-MS B: t_{R}=0.58 min; [M+H]⁺=190.21.

Step 3: Phosphorous tribromide (2.16 mL, 22.7 mmol) is added to a RT soln. of 6-methoxy-8-methylquinolin-4-ol (3.91 g, 20.7 mmol) in DMF (75 mL) and the RM is heated to 45° C. for 1h. The RM is cooled to RT, diluted with water and the pH is adjusted to 8 by the addition of sat. aq. NaHCO₃ soln. The precipitate is collected by filtration and dissolved in EtOAc, washed with brine, dried over Na₂SO₄, filtered and evaporated in vacuo. The crude product is purified by FC (eluting with 10% EtOAc in hept) to give 4-bromo-6-methoxy-8-methylquinoline as a white solid. LC-MS B: t_{R}=0.82 min; [M+H]⁺=254.03.

Step 4: nBuLi (1.6 M in hex, 35.7 mL, 57.1 mmol) is added dropwise to a −78° C. soln. of 4-bromo-6-methoxy-8-methylquinoline (7.2 g, 28.5 mmol) in THF and the RM is stirred for 30 min. The reaction is quenched with sat. aq. NH₄Cl soln. and extracted with EtOAc (3×). The combined org. extracts are washed with brine, dried over Na₂SO₄, filtered and evaporated in vacuo. The crude product is purified by FC (eluting with 20% EtOAc in hept) to give 6-methoxy-8-methylquinoline as a yellow oil. LC-MS B: t_{R}=0.49 min; [M+H]⁺=174.26.

Step 5: 5-Bromo-6-methoxy-8-methylquinoline is prepared from 6-methoxy-8-methylquinoline in analogy to the procedure described for B-4.7 step 1. LC-MS B: t_{R}=0.74 min; [M+H]⁺=252.09.

Step 6: BBr₃ (1 M in DCM, 42.5 mL, 42.5 mmol) is added dropwise to a 0° C. soln. of 5-Bromo-6-methoxy-8-methylquinoline (3.57 g, 14.2 mmol) in DCM (70 mL). The cooling bath is removed and the RM is stirred at RT for 2 h. The RM is carefully quenched into cold MeOH and concentrated in vacuo. The residue is co-evaporated with PhMe, EtOAc and DCM to give 5-bromo-8-methylquinolin-6-ol as a yellow solid. LC-MS B: t_{R}=0.55 min; [M+H]⁺=238.01.

Step 7: tert-Butyl (R)-(1-((5-bromo-8-methylquinolin-6-yl)oxy)-3-phenylpropan-2-yl)carbamate is prepared from 5-bromo-8-methylquinolin-6-ol and tert-butyl (R)-(1-hydroxy-3-phenylpropan-2-yl)carbamate in analogy to the procedure described for B-1.1 Step 2. LC-MS B: t_{R}=1.10 min; [M+H]⁺=472.94.

Step 8: nBuLi (1.6 M in hex, 0.54 mL, 0.86 mmol) is added dropwise to a −78° C. soln. of tert-butyl (R)-(1-((5-bromo-8-methylquinolin-6-yl)oxy)-3-phenylpropan-2-yl)carbamate (185 mg, 0.39 mmol) in THF (2 mL) and the RM is stirred for 30 min before benzyl chloroformate (0.058 mL, 0.41 mmol) is added dropwise. The RM is warmed to RT and quenched by addition of sat. aq. NaHCO₃ and extracted with EtOAc. The layers are separated and the aq. phase is re-extracted with EtOAc (2×) and the combined org. layers are washed with brine, dried over Na₂SO₄, filtered and evaporated in vacuo. The crude product is purified by prep. HPLC (acidic) to give benzyl (R)-6-(2-((tert-butoxycarbonyl)amino)-3-phenylpropoxy)-8-methylquinoline-5-carboxylate as a white solid. LC-MS B: t_{R}=1.11 min; [M+H]⁺=527.33.

Step 9: TFA (4.0 mL, 52.2 mmol) is added to a RT soln. of benzyl (R)-6-(2-((tert-butoxycarbonyl)amino)-3-phenylpropoxy)-8-methylquinoline-5-carboxylate (550 mg, 1.04 mmol) in DCM (5 mL) and the RM is stirred for 1 h. The RM is concentrated in vacuo and the residue is co-evaporated with DCM (2×) before being purified by prep. HPLC (Basic) to give the title compound as a yellow oil. LC-MS B: t_{R}=0.78 min; [M+H]⁺=427.23.

### Benzyl (R)-4-(2-amino-3-phenylpropoxy)-2-methoxy-6-methylnicotinate (B-4.15)

Step 1: NaOH 16% aq. soln. (100 mL, 472 mmol) is added to a RT soln. of ethyl (R)-4-(2-((tert-butoxycarbonyl)amino)-3-phenylpropoxy)-2-methoxy-6-methylnicotinate (B-4.12, step 5) (42 g, 94.5 mmol) in MeOH (300 mL) and heated to 90° C. for 4 h. The RM is cooled to RT and concentrated in vacuo. The remaining aq. phase is extracted with iPrOAc (3×) and the org. phases are discarded. The aq. phase is cooled to 0° C. and acidified with 1M aq. HCl soln. and the precipitate is filtered and washed with water. The solids are dissolved in DCM and remaining water is separated before the org. phase is dried over Na₂SO₄, filtered and evaporated in vacuo to give (R)-4-(2-((tert-butoxycarbonyl)amino)-3-phenylpropoxy)-2-methoxy-6-methylnicotinic acid as a yellow oil. LC-MS B: t_{R}=0.94 min; [M+H]⁺=417.20.

Step 2: K₂CO₃ (1.33 g, 9.6 mmol) and BnBr (0.51 mL, 4.3 mmol) are added to a RT soln. of (R)-4-(2-((tert-butoxycarbonyl)amino)-3-phenylpropoxy)-2-methoxy-6-methylnicotinic acid (2.0 g, 4.8 mmol) in DMF (8 mL) and the RM is heated to 40° C. and stirred for 4 h. The RM is poured into water and extracted with TBME (2×). The combined org. extracts are washed with water, dried over Na₂SO₄, filtered and evaporated in vacuo to give benzyl (R)-4-(2-((tert-butoxycarbonyl)amino)-3-phenylpropoxy)-2-methoxy-6-methylnicotinate as a colourless oil. LC-MS B: t_{R}=1.17 min; [M+H]⁺=507.24.

Step 3: The title compound is prepared from benzyl (R)-4-(2-((tert-butoxycarbonyl)amino)-3-phenylpropoxy)-2-methoxy-6-methylnicotinate in analogy to the procedure described for B-1.1 step 3. Note: Boc-cleavage is performed using TFA instead of HCl and the title compound is isolated as its free base after a basic workup. LC-MS B: t_{R}=0.79 min; [M+H]⁺=407.22.

### Benzyl (R)-4-(2-amino-3-phenylpropoxy)-6-methoxy-2-methylpyrimidine-5-carboxylate trifluoroacetate (B-4.16)

Step 1: nBuLi (1.6 M in hex, 34.5 mL, 55.2 mmol) is added dropwise to a −78° C. soln. of DIPEA (7.74 mL 55.2 mmol) in THF (35 mL) and the RM is stirred for 5 min. A soln. of 4,6-dichloro-2-methylpyrimidine (5.0 g, 30.7 mmol) in THE (40 mL) is added dropwise to the freshly prepared LDA and stirring is continued at −78° C. for 1 h. The RM is quenched with freshly ground dry ice (20 g, 454 mmol) and stirred for 5 min before being warmed to RT over 20 min and stirred for a further 15 min. The RM is concentrated in vacuo to give lithium 4,6-dichloro-2-methylpyrimidine-5-carboxylate as a brown solid which is used further without purification. LC-MS J: t_{R}=0.16 min; [M−H]⁻=204.9.

Step 2: KHCO₃ (6.15 g, 61.4 mmol) and BnBr (10.95 mL, 92 mmol) are added to a RT soln. of lithium 4,6-dichloro-2-methylpyrimidine-5-carboxylate (6.54 g, 30.7 mmol) in DMF (50 mL) and the RM is stirred for 18 h. The RM is quenched by the addition of H₂O and brine and extracted with EtOAc (3×). The combined org. extracts are washed with brine, dried over Na₂SO₄, filtered and evaporated in vacuo. The crude product is purified by FC (eluting with 0% to 5% EtOAc in hept) to give benzyl 4,6-dichloro-2-methylpyrimidine-5-carboxylate as a colourless oil. LC-MS J: t_{R}=2.13 min; [M+H]⁺=296.9.

Step 3: NaOMe (30% soln. in MeOH, 1.0 mL, 5.41 mmol) is added dropwise to a 0° C. soln. of benzyl 4,6-dichloro-2-methylpyrimidine-5-carboxylate (2.68 g, 5.41 mmol) in THF (15 mL) and the RM is stirred for 1 h. The RM is quenched with 1M aq. HCl and extracted with EtOAc (3×). The combined org. extracts are washed with brine, dried over Na₂SO₄, filtered, and evaporated in vacuo. The crude product is purified by FC (eluting with 0% to 7% EtOAc in hept) to give benzyl 4-chloro-6-methoxy-2-methylpyrimidine-5-carboxylate as a colourless oil. LC-MS J: t_{R}=2.15 min; [M+H]⁺=293.1.

Step 4: NaH (60% dispersion in mineral oil, 128 mg, 3.21 mmol) is added to a 0° C. soln. of allyl alcohol (0.21 mL, 3.1 mmol) in THF (10 mL) and the resulting suspension is stirred for 10 min before being slowly added to a −10° C. soln. of benzyl 4-chloro-6-methoxy-2-methylpyrimidine-5-carboxylate in THF (15 mL) and the RM is stirred for 1 h. The RM is quenched with 1M aq. HCl and extracted with EtOAc (3×). The combined org. extracts are washed with brine, dried over Na₂SO₄, filtered, and evaporated in vacuo. The crude product is purified by FC (eluting with 0% to 10% EtOAc in hept) to give benzyl 4-(allyloxy)-6-methoxy-2-methylpyrimidine-5-carboxylate as a colourless oil. LC-MS J: t_{R}=2.24 min; [M+H]⁺=315.1.

Step 5: Pd(PPh₃)₄ (43.7 mg, 0.038 mmol) is added to a RT soln. (degassed) of benzyl 4-(allyloxy)-6-methoxy-2-methylpyrimidine-5-carboxylate (170 mg, 0.54 mmol) and 1,3-dimethylbarbituric acid (127 mg, 0.81 mmol) in MeCN (10 mL) and the RM is heated to 50° C. for 2.5 h. The RM is filtered and concentrated to give benzyl 4-hydroxy-6-methoxy-2-methylpyrimidine-5-carboxylate as a grey solid. LC-MS J: t_{R}=1.63 min; [M+H]⁺=275.1.

Steps 6&7: The title compound is prepared from benzyl 4-hydroxy-6-methoxy-2-methylpyrimidine-5-carboxylate in analogy to the procedure described for B-1.1 steps 2&3 substituting HCl for TFA in the Boc cleavage step. LC-MS J: t_{R}=2.17 min; [M+H]⁺=408.2.

### Ethyl (R)-4-(2-amino-3-phenylpropoxy)-2,6-dimethoxynicotinate (B-4.17)

Step 1: A soln. of benzyl alcohol (0.82 mL, 7.85 mmol) and KO^{t}Bu (867 mg, 7.5 mmol) in DMF (4 mL) is added to a −78° C. soln. of 2,4,6-trifluoropyridine (1.0 g, 7.14 mmol) in DMF (4 mL) and the RM is stirred for 10 min. The RM is quenched with water and warmed to 0° C. before being filtered. The filter residue is re-crystallised from hept to give 4-(benzyloxy)-2,6-difluoropyridine as a white solid. LC-MS B: t_{R}=0.99 min; [M+H]⁺=222.27.

Step 2: A suspension of 4-(benzyloxy)-2,6-difluoropyridine (1.71 g, 7.56 mmol) in NaOMe (25 wt. % in MeOH, 6.9 mL, 30.2 mmol) is heated to 60° C. for 18 h. The RM is concentrated in vacuo and the residue is partitioned between water and TBME and the layers are separated. The aq. phase is re-extracted with TBME (2×) and the combined org. extracts are washed with brine, dried over Na₂SO₄, filtered and evaporated in vacuo to give 4-(benzyloxy)-2,6-dimethoxypyridine as a colourless oil. LC-MS B: t_{R}=1.02 min; [M+H]⁺=246.29.

Steps 3-6: The title compound is prepared from 4-(benzyloxy)-2,6-dimethoxypyridine in analogy to the procedure described for B-4.12 steps 3-6. Note: Boc-cleavage is performed using TFA instead of HCl and the title compound is isolated as its free base after a basic workup. LC-MS B: t_{R}=0.73 min; [M+H]⁺=361.23.

### Benzyl (S)-6-(2-amino-3-phenylpropoxy)-3-fluoroquinoline-5-carboxylate dihydrochloride (B-4.18)

The title compound is prepared from 3-fluoroquinolin-6-ol in analogy to the procedure described for B-4.7 and using tert-butyl (S)-(1-hydroxy-3-phenylpropan-2-yl)carbamate in the Mitsunobu step. LC-MS I: t_{R}=1.07 min; [M+H]⁺=431.08.

### General Method 5 for the Synthesis of Building Blocks B

### Benzyl (R)-2-(2-amino-3-phenylpropoxy)-6-(methoxy-d3)benzoate hydrochloride (B-5.1)

Step 1: DMAP (120 mg, 0.99 mmol) is added to a 0° C. soln. of 2,6-dihydroxybenzoic acid (3.0 g, 19.7 mmol) in 1,2-dimethoxyethane (15 mL) followed by the dropwise addition of acetone (1.9 mL, 25.8 mmol) and thionyl chloride (1.85 mL, 25.2 mmol) and the RM is stirred for 30 min before being warmed to RT and stirred for 16 h. The RM is quenched by the addition of sat. aq. NaHCO₃ and extracted with Et₂O (4×). The combined org. extracts are washed with brine, dried over Na₂SO₄, filtered and evaporated in vacuo. The crude product is purified by FC (eluting with 0% to 50% EtOAc in hept) to give 5-hydroxy-2,2-dimethyl-4H-benzo[d][1,3]dioxin-4-one as a white solid. LC-MS F: t_{R}=1.93 min; [M+H]⁺=195.1.

Step 2: CD₃I (0.8 mL, 12.9 mmol) is added to a soln. of 5-hydroxy-2,2-dimethyl-4H-benzo[d][1,3]dioxin-4-one (1.76 g, 8.6 mmol) and K₂CO₃, (1.79 g, 12.9 mmol) in DMF (25 mL) and the RM is heated to 50° C. for 1 h. The RM is partitioned between water and EtOAc and the layers are separated. The aq. phase is re-extracted with EtOAc (1×) and the combined org. layers are washed with brine, dried over Na₂SO₄, filtered and evaporated in vacuo. The crude product is purified by FC (eluting with 10% to 100% EtOAc in hept) to give 5-(methoxy-d3)-2,2-dimethyl-4H-benzo[d][1,3]dioxin-4-one as an off-white solid. LC-MS J: t_{R}=1.82 min; [M+H]⁺=212.1.

Step 3: NaH (0.65 g, 16.3 mmol) is added to a soln. of benzyl alcohol (1.7 mL, 16.3 mmol) in DMF (45 mL) and the RM is stirred for 30 min before a soln. of 5-(methoxy-d3)-2,2-dimethyl-4H-benzo[d][1,3]dioxin-4-one (1.72 g, 8.1 mmol) in DMF (5 mL) is added and stirring continued for 1 h. The RM is partitioned between 1N HCl and EtOAc and the layers are separated. The aq. phase is re-extracted with EtOAc (1×) and the combined org. layers are washed with brine, dried over Na₂SO₄, filtered and evaporated in vacuo. The crude product is purified by FC (eluting with 0% to 50% EtOAc in hept) to give benzyl 2-hydroxy-6-(methoxy-d3)benzoate as a colourless oil. LC-MS J: t_{R}=2.13 min; [M+H]⁺=262.1.

Step 4: Benzyl (R)-2-(2-((tert-butoxycarbonyl)amino)-3-phenylpropoxy)-6-(methoxy-d3)benzoate is prepared from benzyl 2-hydroxy-6-(methoxy-d3)benzoate and tert-butyl (R)-(1-hydroxy-3-phenylpropan-2-yl)carbamate in analogy to the procedure described for B-1.1 Step 2. LC-MS J: t_{R}=2.39 min; [M-Boc+H]⁺=395.2.

Step 5: The title compound is prepared in analogy to the procedure described for B-1.1 Step 3. LC-MS J: t_{R}=2.15 min; [M+H]⁺=395.1.

### Benzyl (R)-2-(2-amino-3-phenylpropoxy)-6-(difluoromethoxy)benzoate hydrochloride (B-5.2)

Step 1: A soln. of KOH (4.57 g, 81 mmol) in water (12 mL) is added to a 0° C. soln. of 5-hydroxy-2,2-dimethyl-4H-benzo[d][1,3]dioxin-4-one (B-5.1 Step 1, 1.58 g, 8.1 mmol) in MeCN (12 mL) and the biphasic RM is stirred for 5 min before bromodifluoromethyl diethylphosphonate (2.0 mL, 11.4 mmol) is added dropwise. After stirring for 1.5 h EtOAc (25 mL) is added and the phases are separated. The aq. phase is re-extracted with EtOAc (1×) and the combined org. layers are washed with brine, dried over Na₂SO₄, filtered and evaporated in vacuo to give 5-(difluoromethoxy)-2,2-dimethyl-4H-benzo[d][1,3]dioxin-4-one as a brown oil. LC-MS J: t_{R}=2.0 min; [M+H]⁺=245.1.

Steps 2-4: The title compound is prepared from 5-(difluoromethoxy)-2,2-dimethyl-4H-benzo[d][1,3]dioxin-4-one following the sequence of reactions described for B-5.1. LC-MS J: t_{R}=2.20 min; [M+H]⁺=428.1.

### Methyl (R)-6-(2-amino-3-phenylpropoxy)-2-methylbenzofuran-7-carboxylate hydrochloride (B-5.3)

Step 1: K₂CO₃ (2.38 g, 17.2 mmol) and 3-bromopropyne (80% soln. in PhMe, 1.67 mL, 15.5 mmol) are added to a RT soln. of 5-hydroxy-2,2-dimethyl-4H-benzo[d][1,3]dioxin-4-one (B-5.1 Step 1, 3.0 g, 15.4 mmol) in acetone (60 mL) and the RM is heated to 55° C. for 21 h. The mix. is concentrated, and the residue partitioned between water and EtOAc. The layers are separated and the aq. layer re-extracted with EtOAc (2×). The combined org. extracts are washed with brine, dried over Na₂SO₄, filtered, and evaporated in vacuo. The crude product is purified by FC (eluting with 5% to 35% EtOAc in hept) to give 2,2-dimethyl-5-(prop-2-yn-1-yloxy)-4H-benzo[d][1,3]dioxin-4-one as a white solid. LC-MS J: t_{R}=1.83 min; [M+H]⁺=233.1.

Step 2: NaOMe (30% soln. in MeOH, 1.9 mL, 10.1 mmol) is added to a 0° C. soln. of 2,2-dimethyl-5-(prop-2-yn-1-yloxy)-4H-benzo[d][1,3]dioxin-4-one (1.53 g, 6.6 mmol) in DMF (15 mL) and the RM is warmed to RT and stirred for 1 h. The RM is quenched with 1M aq. HCl and extracted with EtOAc (3×). The combined org. extracts are washed with brine, dried over Na₂SO₄, filtered, and evaporated in vacuo to give methyl 2-hydroxy-6-(prop-2-yn-1-yloxy)benzoate as a beige solid. LC-MS J: t_{R}=1.79 min; [M+H]⁺=207.0.

Step 3: A mix. of methyl 2-hydroxy-6-(prop-2-yn-1-yloxy)benzoate (1.33 g, 6.5 mmol), CsF (1.5 g, 9.9 mmol), and diethylaniline (18 mL) is purged with N₂ before being irradiated in a MW oven at 200° C. for 55 min. The RM is diluted with EtOAc and washed with 1M aq. HCl. The aq. phase is extracted with EtOAc (2×) and the combined org. extracts are washed with 1M HCl, brine, dried over Na₂SO₄, filtered, and evaporated in vacuo. The crude product is purified by FC (eluting with 1% to 15% EtOAc in hept) to give methyl 6-hydroxy-2-methylbenzofuran-7-carboxylate as a white solid. LC-MS F: t_{R}=1.98 min; [M+H]⁺=207.0.

Steps 4-5: The title compound is prepared from methyl 6-hydroxy-2-methylbenzofuran-7-carboxylate in analogy to the procedure described for B-1.1 steps 2-3. LC-MS J: t_{R}=1.98 min; [M+H]⁺=340.1.

### General Method 6 for the Synthesis of Building Blocks B

### Methyl (R)-6-(2-amino-3-phenylpropoxy)-3-methylbenzo[d]isoxazole-7-carboxylate hydrochloride (B-6.1)

Step 1: Hexamethylenetetramine (8.1 g, 57.1 mmol) is added to a soln. of 3-methyl-1,2-benzisoxazol-6-ol (2.0 g, 13.4 mmol) in AcOH (40 mL) and the RM is heated to 100° C. for 2 h. 2M aq. HCl (40 mL) is added and stirring is continued at 100° C. for 30 min. The RM is cooled with an ice bath and the resulting solids are collected by filtration. The filtrate is concentrated in vacuo and re-cooled to 0° C. before the solids are again collected by filtration. Both crops are combined and dried in vacuo to give 6-hydroxy-3-methylbenzo[d]isoxazole-7-carbaldehyde as a beige powder. LC-MS B: t_{R}=0.72 min; No ionisation. ¹H NMR (DMSO) δ: 11.67 (s, 1H), 10.43 (s, 1H), 7.94 (d, J=8.7 Hz, 1H), 7.03 (d, J=8.7 Hz, 1H).

Step 2: 2-Methyl-2-butene (7.33 mL, 69.2 mmol) is added in one portion to a RT soln. of 6-hydroxy-3-methylbenzo[d]isoxazole-7-carbaldehyde (1.09 g, 6.1 mmol) in THF (40 mL) and tert-butanol (12 mL) followed by a soln. of NaClO₂ (2.06 g, 18.2 mmol) and NaH₂PO₄.2H₂O (4.3 g, 27.3 mmol) in H₂O (12 mL) and the RM is stirred at RT for 30 min. The solids are collected by filtration, washed with cold 1M aq. HCl and dried in vacuo to give 6-hydroxy-3-methylbenzo[d]isoxazole-7-carboxylic acid as a white solid. LC-MS B: t_{R}=0.63 min; [M+H]⁺=194.31.

Steps 3-5: The title compound is prepared from 6-hydroxy-3-methylbenzo[d]isoxazole-7-carboxylic acid following the sequence of reactions described for B-3.1. LC-MS B: t_{R}=0.70 min; [M+H]⁺=341.38.

### Methyl (R)-5-(2-amino-3-phenylpropoxy)-1-methyl-1H-indazole-4-carboxylate hydrochloride (B-6.2)

The title compound is prepared from 1-methyl-1H-indazol-5-ol following the sequence of reactions described for B-6.1. LC-MS B: t_{R}=0.70 min; [M+H]⁺=340.36.

### General Method 7 for the Synthesis of Building Blocks B

### Methyl (R)-6-(2-amino-3-phenylpropoxy)isoquinoline-5-carboxylate dihydrochloride (B-7.1)

Step 1: Br₂ (0.78 mL, 15.2 mmol) is added dropwise to a suspension of isoquinolin-6-ol (2.0 g, 13.8 mmol) in CHCl₃ (30 mL) in a water bath and the RM is stirred for 2 h. EtOAc is added and the solids are collected by filtration and washed with EtOAc and then hept. The filter residue is neutralised by suspending in sat. aq. NaHCO₃ and re-filtered before washing with H₂O and then hept. The filter residue is suspended in MeCN and evaporated in vacuo to give 5-bromoisoquinolin-6-ol as a brown solid. LC-MS J: t_{R}=0.33 min; [M+H]⁺=224.0.

Step 2: tert-Butyl (R)-(1-((5-bromoisoquinolin-6-yl)oxy)-3-phenylpropan-2-yl)carbamate is prepared from 5-bromoisoquinolin-6-ol and tert-butyl (R)-(1-hydroxy-3-phenylpropan-2-yl)carbamate in analogy to the procedure described for B-1.1 Step 2. LC-MS J: t_{R}=2.28 min; [M+H]⁺=457.1.

Step 3: A soln. of DIPEA (0.76 mL, 4.4 mmol) in MeOH (3 mL) is purged with Ar before Pd(OAc)₂ (74 mg, 0.33 mmol) and Xantphos (190 mg, 0.33 mmol) are added and the catalyst mix. is heated to 70° C. for 20 min. In a separate flask a soln. of tert-butyl (R)-(1-((5-bromoisoquinolin-6-yl)oxy)-3-phenylpropan-2-yl)carbamate (1.0 g, 2.19 mmol) in MeOH (40 mL) is first purged with Ar before being purged with CO and then heated to 70° C. under a CO atm before the hot catalyst soln. is added via syringe and the RM is stirred for 20 h. The RM is cooled to RT and concentrated in vacuo and the residue is partitioned between sat. aq. NaHCO₃ and DCM and extracted. The layers are separated and the aq. phase is re-extracted with DCM (1×) and the combined org. layers are washed with brine, dried over Na₂SO₄, filtered and evaporated in vacuo. The crude product is purified by FC (eluting with 25% to 80% EtOAc in hept) to give methyl (R)-6-(2-((tert-butoxycarbonyl)amino)-3-phenylpropoxy)isoquinoline-5-carboxylate as a black solid. LC-MS J: t_{R}=2.11 min; [M+H]⁺=437.2.

Step 4: The title compound is prepared from methyl (R)-6-(2-((tert-butoxycarbonyl)amino)-3-phenylpropoxy)isoquinoline-5-carboxylate in analogy to the procedure described for B-1.1 Step 3. LC-MS J: t_{R}=1.83 min; [M+H]⁺=337.2.

### Benzyl (R)-6-(2-amino-3-phenylpropoxy)-3-methylisoquinoline-5-carboxylate dihydrochloride (B-7.2)

Step 1: Trifluoromethanesulfonic anhydride (26.2 mL, 158 mmol) is added dropwise to a −10° C. soln. of 2-hydroxy-4-methoxybenzaldehyde (16 g, 105 mmol) and pyridine (42.5 mL, 526 mmol) in DCM (70 mL) and the RM is stirred for 30 min. The RM is quenched with ice water and acidified with 1M aq. HCl before being extracted with EtOAc (2×). The combined org. extracts are washed with brine, dried over Na₂SO₄, filtered and evaporated in vacuo to give 2-formyl-5-methoxyphenyl trifluoromethanesulfonate as a yellow oil. ¹H NMR (400 MHz, CDCl₃) δ 10.13 (s, 1H), 7.95 (d, J=8.8 Hz, 1H), 7.03 (dd, J=8.7, 2.3 Hz, 1H), 6.88 (d, J=2.3 Hz, 1H), 3.93 (s, 3H).

Step 2: A RT soln. of 2-formyl-5-methoxyphenyl trifluoromethanesulfonate (19.6 g, 66.4 mmol) and TEA (93 mL, 664 mmol) in DMF (400 mL) is purged with Ar for 30 min. Prop-1-yne (1 M in DMF, 133 mL, 133 mmol), CuI (1.27 g, 6.64 mmol) and Pd(PPh₃)₄ (5.0 g, 4.33 mmol) are added successively and the RM is stirred closed for 2 h. The RM is filtered through a pad of celite and the filtrate partially concentrated in vacuo before being diluted with EtOAc and washed successively with 1M KHSO₄ soln. and brine and concentrated in vacuo. The crude product is purified by FC (eluting with 0% to 30% EtOAc in hept) to give 4-methoxy-2-(prop-1-yn-1-yl)benzaldehyde as a yellow solid. LC-MS J: t_{R}=1.80 min; [M+H]⁺=175.1.

Step 3: A RT soln. of 4-methoxy-2-(prop-1-yn-1-yl)benzaldehyde (10.3 g, 58.8 mmol) in MeOH (350 mL) is purged with Ar for 5 min in an autoclave. NH₃ 7M in MeOH (150 mL, 1050 mmol) is added and the RM is heated to 65° C. at 2 bar for 4 h. The RM is concentrated in vacuo and the residue is co-evaporated with DCM (2×) to give 6-methoxy-3-methylisoquinoline as a brown solid. LC-MS J: t_{R}=1.81 min; [M+H]⁺=174.1.

Step 4: BBr₃ (1 M in DCM, 55.4 mL, 55.4 mmol) is added dropwise to a −78° C. soln. of 6-methoxy-3-methylisoquinoline (5.0 g, 27.7 mmol) in DCM (100 mL). The cooling bath is removed and the RM is stirred at RT for 30 h. The RM is carefully quenched into cold MeOH and concentrated in vacuo. The residue is co-evaporated with PhMe, EtOAc and DCM to give 3-methylisoquinolin-6-ol as a brown solid. LC-MS J: t_{R}=1.10 min; [M+H]⁺=160.1.

Steps 5-6: tert-Butyl (R)-(1-((5-bromo-3-methylisoquinolin-6-yl)oxy)-3-phenylpropan-2-yl)carbamate is prepared from 3-methylisoquinolin-6-ol following steps 1&2 described for B-7.1. LC-MS J: t_{R}=2.20 min; [M+H]⁺=471.1.

Step 7: A RT soln. of tert-butyl (R)-(1-((5-bromo-3-methylisoquinolin-6-yl)oxy)-3-phenylpropan-2-yl)carbamate (2.5 g, 5.30 mmol), benzyl alcohol (2.76 mL, 26.5 mmol) and DIPEA (2.78 mL, 15.9 mmol) in PhMe (20 mL) is purged with Ar for 10 min. The RM is then purged with CO and heated to 88° C. under a CO atm before a soln. of Pd(^{t}Bu₃P)₂ (271 mg, 0.53 mmol) in PhMe (5.5 mL) is added via syringe pump (3 mL/h). The temperature is increased to 95° C. and the RM is stirred under a CO atm for 24 h. The RM is cooled to RT and concentrated in vacuo and the residue is partitioned between sat. aq. NaHCO₃ and EtOAc and extracted. The layers are separated and the aq. phase is re-extracted with EtOAc (1×) and the combined org. layers are washed with brine, dried over Na₂SO₄, filtered and evaporated in vacuo. The crude product is purified by FC (eluting with 5% to 65% EtOAc in hept) to give benzyl (R)-6-(2-((tert-butoxycarbonyl)amino)-3-phenylpropoxy)-3-methylisoquinoline-5-carboxylate as a colourless oil. LC-MS J: t_{R}=2.19 min; [M+H]⁺=527.2.

Step 8: The title compound is prepared from benzyl (R)-6-(2-((tert-butoxycarbonyl)amino)-3-phenylpropoxy)-3-methylisoquinoline-5-carboxylate in analogy to the procedure described for B-1.1 Step 3. LC-MS J: t_{R}=1.95 min; [M+H]⁺=427.2.

### Synthesis of Building Blocks B-Acids

### (R)-6-(2-((tert-Butoxycarbonyl)amino)-3-phenylpropoxy)benzo[d][1,3]dioxole-5-carboxylic acid (B-Acid-1)

Step 1: Benzyl 6-hydroxybenzo[d][1,3]dioxole-5-carboxylate is prepared from 6-hydroxybenzo[d][1,3]dioxole-5-carboxylic acid in analogy to the procedure described for B-1.1 step 1. LC-MS I: t_{R}=1.12 min; [M+H]⁺=272.94.

Step 2: Benzyl (R)-6-(2-((tert-butoxycarbonyl)amino)-3-phenylpropoxy)benzo[d][1,3]dioxole-5-carboxylate is prepared from benzyl 6-hydroxybenzo[d][1,3]dioxole-5-carboxylate in analogy to the procedure described for B-1.1 step 2. LC-MS I: t_{R}=1.27 min; [M+H]⁺=506.02.

Step 3: The title compound is prepared from benzyl (R)-6-(2-((tert-butoxycarbonyl)amino)-3-phenylpropoxy)benzo[d][1,3]dioxole-5-carboxylate in analogy to the procedure described for B-1.24 step 4. LC-MS I: t_{R}=0.53 min; [M+H]⁺=416.01.

### (R)-2-(2-((tert-Butoxycarbonyl)amino)-3-phenylpropoxy)-4,5-dimethoxybenzoic acid (B-Acid-2)

The title compound is prepared from 2-hydroxy-4,5-dimethoxybenzoic acid following the 3-step sequence as described for B-Acid-1. LC-MS B: t_{R}=0.98 min; [M+H]⁺=432.18.

### (R)-2-(2-((tert-Butoxycarbonyl)amino)-3-phenylpropoxy)-6-methoxybenzoic acid (B-Acid-3)

The title compound is prepared from 2-hydroxy-6-methoxybenzoic acid following the 3-step sequence as described for B-Acid-1. LC-MS I: t_{R}=0.52 min; [M+H]⁺=401.88.

### (R)-6-(2-((((9H-Fluoren-9-yl)methoxy)carbonyl)amino)-3-phenylpropoxy)benzo[d][1,3]dioxole-5-carboxylic acid (B-Acid-4)

Step 1: Benzyl (R)-6-(2-(((benzyloxy)carbonyl)amino)-3-phenylpropoxy)benzo[d][1,3]dioxole-5-carboxylate is prepared from benzyl 6-hydroxybenzo[d][1,3]dioxole-5-carboxylate in analogy to the procedure described for B-1.1 step 2 substituting tert-butyl (R)-(1-hydroxy-3-phenylpropan-2-yl)carbamate by benzyl (R)-(1-hydroxy-3-phenylpropan-2-yl)carbamate. LC-MS I: t_{R}=1.27 min; [M+H]⁺=540.20.

Step 2: A soln. of benzyl (R)-6-(2-(((benzyloxy)carbonyl)amino)-3-phenylpropoxy)benzo[d][1,3]dioxole-5-carboxylate (2.4 g, 4.45 mmol) in THF (40 mL) is evacuated/purged with N₂ (3×) before 10% Pd/C (473 mg, 10 mol %) is added. The RM is evacuated/purged with H₂ (3×) and stirred under a H₂ atm for 2 h. The RM is filtered through a pad of celite and the filtrate concentrated in vacuo to give (R)-6-(2-amino-3-phenylpropoxy)benzo[d][1,3]dioxole-5-carboxylic acid as a white solid. LC-MS B: t_{R}=0.65 min; [M+H]⁺=316.13.

Step 3: N-(9-Fluorenylmethoxycarbonyloxy)succinimide (1.45 g, 4.28 mmol) is added to a RT soln. of (R)-6-(2-amino-3-phenylpropoxy)benzo[d][1,3]dioxole-5-carboxylic acid (1.35 g, 4.28 mmol) and Na₂CO₃ (926 mg, 8.56 mmol) in a mix. of dioxane (40 mL) and water (8 mL) and the RM is stirred for 16 h. The RM is concentrated in vacuo and the residue partitioned between 1M HCl and EtOAc and extracted. The layers are filtered and separated, and the aq. phase is re-extracted with EtOAc (2×). The combined org. extracts are washed with brine, dried over Na₂SO₄, filtered and evaporated in vacuo. The crude product is purified by FC (eluting with 0% to 5% MeOH in DCM) to give the title compound as a white solid. LC-MS B: t_{R}=1.09 min; [M+H]⁺=538.24.

### (R)-2-(2-((tert-Butoxycarbonyl)amino)-3-phenylpropoxy)-1-naphthoic acid (B-Acid-5)

The title compound is prepared from 2-hydroxy-1-naphthoic acid following the 3-step sequence as described for B-Acid-1. LC-MS B: t_{R}=1.02 min; [M+H]⁺=422.33.

### (R)-3-(2-((tert-Butoxycarbonyl)amino)-3-phenylpropoxy)quinoline-4-carboxylic acid (B-Acid-6)

The title compound is prepared from 3-hydroxyquinoline-4-carboxylic acid following the 3-step sequence as described for B-Acid-1. LC-MS B: t_{R}=0.88 min; [M+H]⁺=423.35.

### (R)-4-(2-((tert-Butoxycarbonyl)amino)-3-phenylpropoxy)-2-methoxynicotinic acid (B-Acid-7)

The title compound is prepared from 4-hydroxy-2-methoxynicotinic acid following the 3-step sequence as described for B-Acid-1. LC-MS B: t_{R}=0.90 min; [M+H]⁺=403.20.

### (R)-6-(2-(((Allyloxy)carbonyl)amino)-3-phenylpropoxy)-3-fluoroquinoline-5-carboxylic acid (B-Acid-8)

Step 1: Under N₂, benzyl (R)-6-(2-amino-3-phenylpropoxy)-3-fluoroquinoline-5-carboxylate dihydrochloride (B-4.9) (0.46 g, 0.92 mmol) is dissolved in MeOH (10 mL); the vessel is purged with N₂/vacuum (3×) before 10% Pd/C (50 mg) is added. After inertising another three times a H₂ balloon is connected and the RM is stirred for 1 h under H2 atmosphere. The heterogeneous reaction mixture is filtered over a glass fiber filter (washing with methanol/THF). The filtrate is then concentrated to dryness under reduced pressure to yield 0.43 g of (R)-6-(2-amino-3-phenylpropoxy)-3-fluoroquinoline-5-carboxylic acid as a crude yellow oil which is used as such in the next step. LC-MS B: t_{R}=0.62 min; [M+H]⁺=341.21.

Step 2: (R)-6-(2-Amino-3-phenylpropoxy)-3-fluoroquinoline-5-carboxylic acid (436 mg, 1.28 mmol) is taken up in THE (10 mL) and water (10 mL). To the resulting light suspension is added NaHCO₃ (430 mg, 5.12 mmol) followed by allyl chloroformate (0.155 mL, 1.41 mmol). The reaction mixture is stirred for 1 h at RT. The reaction mixture is diluted/partitioned between water and EtOAc and acidified carefully with some HCl (2N) down to pH-3. The layers are separated and the inorg. layer is extracted further with EtOAc (2×). The combined organic extracts are washed with acidified water and brine, dried over Na₂SO₄, filtered and evaporated in vacuo to yield the title compound as a yellow oil (0.39 g). No purification at this stage. LC-MS B: t_{R}=0.94 min; [M+H]⁺=425.23.

### (R)-4-(2-(((Allyloxy)carbonyl)amino)-3-phenylpropoxy)-2-methoxy-6-methylnicotinic acid (B-Acid-9)

The title compound is prepared as a colorless oil from benzyl (R)-4-(2-Amino-3-phenylpropoxy)-2-methoxy-6-methylnicotinate (B-4.15) following the 2-step sequence described for B-Acid-8. LC-MS B: t_{R}=0.89 min; [M+H]⁺=401.07.

### General Method 1 for the Synthesis of Building Blocks C

### N—(N-(tert-Butoxycarbonyl)-N-methyl-L-leucyl)-N-methylglycine (C-1.1)

Step 1: K₂CO₃ (7.15 g, 51.7 mmol) is added to a RT soln. of Boc-N-methylglycine (7.12 g, 36.9 mmol) in acetone (100 mL) followed by the dropwise addition of benzyl bromide (4.93 mL, 40.6 mmol). The resulting mix. is heated to 45° C. and stirred for 16 h. The mix. is cooled to RT before being filtered and the filtrate is concentrated to give benzyl N-(tert-butoxycarbonyl)-N-methylglycinate that is used without purification. LC-MS B: t_{R}=0.99 min; [M+H]⁺=280.36.

Step 2: 4 M HCl in dioxane (34.3 mL, 0.137 mol) is added to a RT soln. of benzyl N-(tert-butoxycarbonyl)-N-methylglycinate (11.0 g, 34.2 mmol) in dioxane (10 mL) and the resulting mix. is stirred for 2 h. The suspension is filtered and washed with Et₂O (2×) to give benzyl methylglycinate HCl as a white solid. LC-MS B: t_{R}=0.48 min; [M+H]+=180.49.

Step 3: A soln. of benzyl methylglycinate HCl (7.96 g, 36.9 mmol) and DIPEA (9.48 mL, 55.4 mmol) in DMF (30 mL) is added to a prestirred RT soln. of Boc-N-methyl-L-leucine (9.07 g, 36.9 mmol), HATU (14.03 g, 36.9 mmol) and DIPEA (9.48 mL, 55.4 mmol) in DMF (70 mL) and the resulting mix. is stirred for 2 h. The mix. is concentrated, and the residue partitioned between water and EtOAc. The layers are separated and the aq. layer re-extracted with EtOAc (2×). The combined org. extracts are washed with brine, dried (MgSO₄), filtered, and evaporated to give the crude product that is triturated with Et₂O to give benzyl N—(N-(tert-butoxycarbonyl)-N-methyl-L-leucyl)-N-methylglycinate as a colourless oil. LC-MS B: t_{R}=1.11 min; [M+H]⁺=407.47.

Step 4: A soln. of benzyl N—(N-(tert-butoxycarbonyl)-N-methyl-L-leucyl)-N-methylglycinate (14.2 g, 34.2 mmol) in EtOH (200 mL) is purged with N₂/vacuum (3×) before 10% Pd/C (1.82 g, 1.7 mmol) is added. After inertising another three times a H₂ balloon is connected and the RM is stirred for 2.5 h. The mix. is concentrated and filtered over a celite plug rinsing with EtOH. The filtrate is concentrated to give the title compound as a colourless oil. LC-MS B: t_{R}=0.84 min; [M+H]⁺=317.31.

### (S)-1-(2-((tert-Butoxycarbonyl) (methyl)amino)-N,4-dimethylpentanamido)cyclopropane-1-carboxylic acid (C-1.2)

Step 1: KtOBu (49.6 g, 0.43 mol) is added to a RT soln. of THF (600 mL). A soln. of 1-(Boc-amino)cyclopropanecarboxylic acid (40.0 g, 0.195 mol) in THF (400 mL) is added to the above suspension, then dimethylsulfate (19.6 mL, 0.205 mol) is added carefully (exothermic) and the RM stirred at RT for 2 h, before dimehtylsulfate (1 mL, 0.01 mol) is added and stirring continued for another 1 h at RT. The reaction is quenched with H₂O (400 mL) and acidified with 32% aq. HCl (80 mL). The layers are separated and the aq. layer is extracted with DCM (500 mL). The combined org. layers are concentrated to a reduced volume, then the org. layer is washed with H₂O (300 mL) before being concentrated. Hept is added to the oily residue and the obtained suspension is left at RT overnight. Next morning, the mix. is filtered and the obtained solid is rinsed with hept (30 mL) and dried to yield 1-(tert-butoxycarbonyl-methyl-amino)-cyclopropanecarboxylic acid (21.5 g, 51%) as a white solid. LC-MS B: t_{R}=0.71 min; [M+H]⁺=216.39.

Step 2-5: The title compound is prepared from 1-(tert-butoxycarbonyl-methyl-amino)-cyclopropanecarboxylic acid following the sequence of reactions described for C-1.1, steps 1-4. LC-MS B: t_{R}=0.87 min; [M+H]⁺=343.26.

### 1-(N-(tert-Butoxycarbonyl)-N-methyl-L-leucyl)-1,2,3,6-tetrahydropyridine-2-carboxylic acid (C-1.3)

Step 1: H₂SO₄ (92 μL, 1.72 mmol) is added to a 0° C. soln. of (S)—N-Boc-1,2,3,6-tetrahydro-2-pyridinecarboxylic acid (400 mg, 1.72 mmol) in MeOH (5 mL) and the resulting mix. is heated to reflux for 4 h. The RM is slowly poured into a 0° C. soln. of sat. NaHCO₃ and extracted with EtOAc (3×). The combined org. extracts are dried (MgSO₄), filtered, and concentrated to give methyl (S)-1,2,3,6-tetrahydropyridine-2-carboxylate as an orange oil. LC-MS I: t_{R}=0.46 min; [M+H]⁺=142.16.

Steps 2&3: The title compound is prepared from Boc-N-methyl-L-leucine and (S)-1,2,3,6-tetrahydropyridine-2-carboxylate following the sequence of reactions described for C-2.1. LC-MS B: t_{R}=0.92 min; [M+H]⁺=355.34. Epimerisation of the tetrahydropyridine 2-position was observed and this building block is used further as a mix. of diastereoisomers.

### N—(N-(tert-Butoxycarbonyl)-N-methyl-L-leucyl)-N-methyl-D-alanine (C-1.4)

Step 1: Mel (1.0 mL, 16.06 mmol) is added to a 0° C. soln. of (tert-butoxycarbonyl)-D-alanine (2.01 g, 10.62 mmol) in THF (10 mL), then NaH (1.08 g, 27.1 mmol) is added. After 30 min at 0° C., the RM is warmed to RT and stirring is continued for 3 h. The mix. is quenched with water and acidified with 0.5 M KHSO₄ (pH 2). The layers are separated and the aq. layer is extracted with EtOAc (3×20 mL). The combined org. layers are washed with brine, dried (Na₂SO₄), filtered, and evaporated to yield N-(tert-butoxycarbonyl)-N-methyl-D-alanine as a brown oil which is used as such in the next step.

Steps 2-5: The title compound is prepared from N-(tert-butoxycarbonyl)-N-methyl-D-alanine following the 4-step sequence of reactions described for C-1.1, steps 1-4. LC-MS F: t_{R}=1.98 min; [M+H]⁺=331.20. ¹H NMR (400 MHz, DMSO) δ 5.03-4.46 (m, 2H), 2.95-2.83 (m, 2H), 2.75-2.54 (m, 4H), 1.58-1.44 (m, 2H), 1.41 (s, 10H), 1.31-1.23 (m, 3H), 1.23-1.17 (m, 1H), 0.94-0.84 (m, 6H).

### General Method 2 for the Synthesis of Building Blocks C

### (R)-4-(N-(tert-Butoxycarbonyl)-N-methyl-L-leucyl) morpholine-3-carboxylic acid (C-2.1)

Step 1: HATU (4.64 g, 12.2 mmol) is added portionwise to a RT soln. of Boc-N-methyl-L-leucine (3.0 g, 12.2 mmol), (R)-methyl morpholine-3-carboxylate (1.85 g, 12.2 mmol), and DIPEA (6.3 mL, 36.6 mmol) in DMF (30 mL) and the resulting mix. is stirred for 1 h. Water is added and the mix. is extracted with EtOAc (3×). The combined org. extracts are successively washed with sat. aq. NaHCO₃, water, and brine, dried (Na₂SO₄), filtered, and concentrated. Purification by FC (eluting with 0% to 100% EtOAc in hept) gives methyl (R)-4-(N-(tert-butoxycarbonyl)-N-methyl-L-leucyl)morpholine-3-carboxylate as a colourless oil. LC-MS B: t_{R}=0.97 min; [M+H]⁺=373.50.

Step 2: 2 M aq. NaOH (11.7 mL, 22.6 mmol) is added to a RT soln. of methyl (R)-4-(N-(tert-butoxycarbonyl)-N-methyl-L-leucyl)morpholine-3-carboxylate (4.37 g, 11.7 mmol) in MeOH (55 mL) and the mix. is stirred at RT for 4.5 h. The volatiles are removed in vacuo and the aq. residue is neutralised with 2 M aq. HCl before being extracted with DCM (3×). The combined org. layers are dried (Na₂SO₄), filtered, and evaporated in vacuo to give the title compound as a white solid. LC-MS B: t_{R}=0.86 min; [M+H]⁺=359.49.

Listed in Table C-2 below are building blocks C that are prepared from the corresponding starting materials in analogy to the 2-step sequence described above for C-2.1.

TABLE C-2

| | | | | t_{R} [min] | MS-data |
|---|---|---|---|---|---|
| | | | | LC-MS | m/z |
| No. | SM A | SM B | Product | Method | [M + H]⁺ |
| C-2.2 | Boc-N- | (R)-Piperidine-2-carboxylic acid | (R)-1-(N-(tert- | 0.93 | 357.51 |
| | Methyl-L- | methyl ester HCl | Butoxycarbonyl)-N-methyl- | B | |
| | leucine | | L-leucyl)piperidine-2- | | |
| | | | carboxylic acid | | |
| C-2.3 | Boc-N- | (S)-Methyl pyrrolidine-3- | (S)-1-(N-(tert- | 0.82 | 343.51 |
| | Methyl-L- | carboxylate HCl | Butoxycarbonyl)-N-methyl- | B | |
| | leucine | | L-leucyl)pyrrolidine-3- | | |
| | | | carboxylic acid | | |
| C-2.4 | Boc-N- | Methyl 3-methoxy-1- | (S)-1-(2-((tert- | 0.89 | 387.24 |
| | Methyl-L- | (methylamino)cyclobutane-1- | Butoxycarbonyl)(methyl)amino)- | B | |
| | leucine | carboxylate HCl | N,4-dimethylpentanamido)-3- | | |
| | | as a mix. of stereoisomers | methoxycyclobutane-1- | | |
| | | Aurum Pharmatech TR33962 | carboxylic acid | | |
| C-2.5 | D2-1.1 | (R)-Piperidine-2-carboxylic acid | (R)-1-((S)-2-((tert- | 0.98 | 383.42 |
| | | methyl ester HCI | Butoxycarbonyl)amino)-3- | B | |
| | | | cyclopentylpropanoyl)piperidine- | | |
| | | | 2-carboxylic acid | | |
| C-2.6 | D2-1.1 | (R)-Methyl morpholine-3- | (R)-4-((S)-2-((tert- | 0.92 | 385.25 |
| | | carboxylate | Butoxycarbonyl)(methyl)amino)- | B | |
| | | | 3-cyclopentylpropanoyl)morpholine- | | |
| | | | 3-carboxylic acid | | |
| C-2.7 | Boc-N- | Methyl 1- | (S)-1-(2-((tert- | 0.83 | 329.39 |
| | Methyl-L- | aminocyclopropanecarboxylate | Butoxycarbonyl)(methyl)amino)-4- | B | |
| | leucine | | methylpentanamido)cyclopropane- | | |
| | | | 1-carboxylic acid | | |
| C-2.8 | Boc-N- | (S)-Piperidine-2-carboxylic acid | (S)-1-[(S)-2-(N-(tert- | 0.54 | 357.33 |
| | Methyl-L- | methyl ester HCl | Butoxycarbonyl)-N-methyl- | I | |
| | leucine | | amino)-4-methyl- | | |
| | | | pentanoyl]-L- | | |
| | | | leucyl)piperidine-2- | | |
| | | | carboxylic acid | | |
| C-2.9 | Boc-N- | Methyl 4,4-difluoropiperidine-2- | 1-(N-(tert-Butoxycarbonyl)- | 0.56 | 393.28 |
| | Methyl-L- | carboxylate hydrochloride | N-methyl-L-leucyl)-4,4- | I | |
| | leucine | | difluoropiperidine-2- | | |
| | | | carboxylic acid | | |
| C-2.10 | Boc-N- | 1,2,3,4-Tetrahydro-1- | 2-(N-(tert-Butoxycarbonyl)- | 0.57 | 405.25 |
| | Methyl-L- | isoquinoline carboxylic acid ethyl | N-methyl-L-leucyl)-1,2,3,4- | I | |
| | leucine | ester HCl, CAS 106181-28-6, | tetrahydroisoquinoline-1- | | |
| | | AstaTech Lot C2130-078 | carboxylic acid | | |
| C-2.11 | Boc-N- | 1,2,3,4-Tetrahydro-isoquinoline- | 2-(N-(tert-Butoxycarbonyl)- | 0.56 | 405.31 |
| | Methyl-L- | 3-carboxylic acidmethyl ester | N-methyl-L-leucyl)-1,2,3,4- | I | |
| | leucine | (HCl salt) | tetrahydroisoquinoline-3- | | |
| | | | carboxylic acid | | |
| C-2.12 | Boc-N- | Methyl 2-(methylamino)-2- | 2-((S)-2-((tert- | 0.98 | 393.30 |
| | Methyl-L- | phenyl-acetate (HCl salt) | Butoxycarbonyl)(methyl)amino)- | B | |
| | leucine | | N,4-dimethylpentanamido)-2- | | |
| | | | phenylacetic acid | | |
| C-2.15 | Boc-N- | Methyl 4,4,4-trifluoro-2- | (R)-2-((S)-2-((tert- | 0.56 | 399.27 |
| | Methyl-L- | (methylamino)butanoate (HCl- | Butoxycarbonyl)(methyl)amino)- | I | |
| | leucine | salt) | N,4-dimethylpentanamido)- | | |
| | | | 4,4,4-trifluorobutanoic acid | | |
| C-2.16 | Boc-N- | Methyl 3-cyclobutoxy-2- | N-(N-(tert-Butoxycarbonyl)- | 0.99 | 401.22 |
| | Methyl-L- | (methylamino)propanoate | N-methyl-L-leucyl)-O- | A | |
| | leucine | hydrochloride | cyclobutyl-N-methylserine | | |
| C-2.17 | Boc-N- | Methyl azepane-2-carboxylate | 1-(N-(tert-Butoxycarbonyl)- | 0.95 | 371.30 |
| | Methyl-L- | hydrochloride | N-methyl-L- | A | |
| | leucine | | leucyl)azepane-2- | | |
| | | | carboxylic acid | | |

### 6-(N-(tert-Butoxycarbonyl)-N-methyl-L-leucyl)-6-azaspiro[2.5]octane-5-carboxylic acid (C-2.13)

Step 1: (Trimethylsilyl)diazomethane soln. (2.0 M in hex, 0.75 mL, 1.49 mmol) is added to a 0° C. soln. of commercially available 6-azaspiro[2.5]octane-5-carboxylic acid hydrochloride in MeOH (2 mL). The RM is warmed to RT and stirred for 30 min. The mix. is concentrated to yield rac-6-aza-spiro[2.5]octane-5-carboxylic acid methyl ester which is used as such in the next step. LC-MS I: t_{R}=0.66 min; [M+H]⁺=170.18.

Steps 2&3: The title compound is prepared from rac-6-aza-spiro[2.5]octane-5-carboxylic acid methyl ester following the 2-step procedure described for C-2.1. LC-MS I: t_{R}=1.27 min; [M+H]⁺=397.34.

### O-Benzyl-N—(N-(tert-butoxycarbonyl)-N-methyl-L-leucyl)-N-methylserine (C-2.14)

Step 1: Thionyl chloride is added to a 0° C. solution of O-benzyl-N-methyl-DL-serine (3.10 g, 14.8 mmol) in DCM (20 mL) and the RM is stirred at 60° C. for 16 h. The mix. is poured into ice water and extracted with DCM (3×). The combined organic layers are washed with brine, dried (MgSO4), filtered, and concentrated in vacuo. Purification by FC (eluting with 0% to 20% EtOAc in hept) yields methyl O-benzyl-N-methylserinate as a colourless oil. LC-MS B: t_{R}=0.53 min; [M+H]⁺=224.04.

Steps 2&3: The title compound is prepared from methyl O-benzyl-N-methylserinate following the 2-step procedure described for C-2.1. LC-MS B: t_{R}=1.01 min; [M+H]⁺=437.30.

### General Method 3 for the Synthesis of Building Blocks C

### (R)-1-(N-(tert-Butoxycarbonyl)-N-methyl-L-leucyl)-4-methylpiperazine-2-carboxylic acid (C-3.1)

Step 1: Sodium acetate (3.36 g, 41 mmol), followed by TFA (0.63 mL, 8.2 mmol) and formaldehyde 37% aq. (2.92 mL, 39 mmol) are added to a RT soln. of 1-tert-butyl 2-methyl (2R)-piperazine-1,2-dicarboxylate (2.0 g, 8.2 mmol) in MeOH (40 mL) and the resulting mix. is stirred for 30 min. The mix. is cooled to 0° C. before NaBH₃CN (1.74 g, 26.3 mmol) is added portionwise. The mix. is warmed to RT and stirred for 5 h before being concentrated. The residue is partitioned between EtOAc and 1 M aq. NaOH and extracted. The layers are separated, and the aq. layer re-extracted with EtOAc (2×). The combined org. extracts are washed with brine, dried (Na₂SO₄), filtered, and evaporated to give 1-(tert-butyl) 2-methyl (R)-4-methylpiperazine-1,2-dicarboxylate as a colourless oil. LC-MS I: t_{R}=0.81 min; [M+H]⁺=259.22.

Step 2: Methyl (R)-4-methylpiperazine-2-carboxylate dihydrochloride is prepared from 1-(tert-butyl) 2-methyl (R)-4-methylpiperazine-1,2-dicarboxylate in analogy to the procedure described for C-1.1, step 2. LC-MS I: t_{R}=0.34 min; [M+H]⁺=159.16.

Steps 3&4: The title compound is prepared from Boc-N-methyl-L-leucine and methyl (R)-4-methylpiperazine-2-carboxylate dihydrochloride following the 2-step sequence of reactions described for C-2.1. LC-MS B: t_{R}=0.67 min; [M+H]⁺=372.51.

Listed in Table C-3 below are building blocks C that are prepared from the corresponding starting materials in analogy to the 4-step sequence described above for C-3.1.

TABLE C-3

| | | | t_{R} [min] | MS-data |
|---|---|---|---|---|
| | | | LC-MS | m/z |
| No. | SM | Product | Method | [M + H]⁺ |
| C-3.2 | Acetaldehyde | (R)-1-(N-(tert-Butoxycarbonyl)-N-methyl-L- | 0.56 | 386.00 |
| | | leucyl)-4-ethylpiperazine-2-carboxylic acid | I | |
| C-3.3 | Acetone | (R)-1-(N-(tert-Butoxycarbonyl)-N-methyl-L- | 0.71 | 400.51 |
| | | leucyl)-4-isopropylpiperazine-2-carboxylic acid | B | |
| C-3.4 | Isobutyraldehyde | (R)-1-(N-(tert-Butoxycarbonyl)-N-methyl-L- | 0.75 | 414.45 |
| | | leucyl)-4-isobutylpiperazine-2-carboxylic acid | B | |
| C-3.5 | Cyclobutanone | (R)-1-(N-(tert-Butoxycarbonyl)-N-methyl-L- | 0.73 | 412.48 |
| | | leucyl)-4-cyclobutylpiperazine-2-carboxylic | B | |
| | | acid | | |
| C-3.6 | Cyclopropanone | (R)-1-(N-(tert-Butoxycarbonyl)-N-methyl-L- | 0.56 | 412.30 |
| | | leucyl)-4-(cyclopropylmethyl)piperazine-2- | I | |
| | | carboxylic acid | | |

### (R)-1-(N-(tert-Butoxycarbonyl)-N-methyl-L-leucyl)-4-cyclopropylpiperazine-2-carboxylic acid (HCl-salt) (C-3.7)

Step 1: (1-Ethoxycyclopropoxy)trimethylsilane (2.6 mL, 12.80 mmol), NaBH₃CN (0.66 g, 9.98 mmol), and AcOH (0.5 mL, 8.74 mmol) are added to a RT soln. of methyl (R)-1-Boc-piperazine-2-carboxylate (1.54 g, 6.30 mmol) in MeOH (30 mL) and THF (30 mL) and the resulting mix. is heated to 60° C. for 16 h. Water (5 mL) is added to the cooled mix. followed by 1 M aq. NaOH (10 mL) and after stirring for 15 min the volatiles are removed under reduced pressure. The residue is extracted with DCM (2×) and the combined org. extracts are washed with brine, dried (Na₂SO₄), filtered, and evaporated to give 1-(tert-butyl) 2-methyl (R)-4-cyclopropylpiperazine-1,2-dicarboxylate as a colourless oil. LC-MS B: t_{R}=0.58 min; [M+H]⁺=285.31.

Steps 2-4: The title compound is prepared from 1-(tert-butyl) 2-methyl (R)-4-cyclopropylpiperazine-1,2-dicarboxylate following 3-step sequence as described for C-3.1, steps 2 to 4. LC-MS B: t_{R}=0.73 min; [M+H]⁺=398.43.

### General Method 4 for the Synthesis of Building Blocks C

### N—(N-(tert-Butoxycarbonyl)-N-methyl-L-leucyl)-N-(chroman-3-ylmethyl)glycine (C-4.1)

Step 1: HATU (219 mg, 0.58 mmol) is added to a RT soln. of Boc-N-methyl-L-leucine (135 mg, 0.55 mmol), IM-1.3 (155 mg, 0.5 mmol), and DIPEA (0.34 mL, 2 mmol) in DMF (2 mL) and the resulting mix. is stirred for 1 h. The RM is directly purified by prep. HPLC (basic) to yield benzyl N—(N-(tert-butoxycarbonyl)-N-methyl-L-leucyl)-N-((chroman-3-yl)methyl)glycinate (218 mg, 81%). LC-MS I: t_{R}=1.35 min; [M+H]⁺=539.23.

Step 2: LiOH (52.9 mg, 1.26 mmol) is added to a RT soln. of benzyl N—(N-(tert-butoxycarbonyl)-N-methyl-L-leucyl)-N-((chroman-3-yl)methyl)glycinate (218 mg, 0.4 mmol) in THF/H₂O (2:1) (2 mL) and the mix. is stirred at RT overnight. The volatiles are removed in vacuo and the aq. residue is acidified with 2 M aq. HCl before being extracted with EtOAc (3×). The combined org. layers are dried (MgSO₄), filtered, and evaporated to give the title compound C-4.1 (200 mg, 71%) as a white solid. LC-MS I: t_{R}=0.62 min; [M+H]⁺=449.25.

Listed in Table C-4 below are building blocks C that are prepared from the corresponding starting materials in analogy to the 2-step sequence described above for C-4.1.

TABLE C-4

| | | | | t_{R} [min] | MS-data |
|---|---|---|---|---|---|
| | | | | LC-MS | m/z |
| No. | SM A | SM B | Product | Method | [M + H]⁺ |
| C-4.2 | Boc-N- | IM-1.1 | N-(2-(tert-Butoxy)ethyl)-N-(N-(tert- | 0.99 | 403.46 |
| | Methyl-L- | | butoxycarbonyl)-N-methyl-L-leucyl)glycine | B | |
| | leucine | | | | |
| C-4.3 | Boc-N- | IM-1.2 | N-(N-(tert-Butoxycarbonyl)-N-methyl-L-leucyl)-N- | 0.93 | 402.26 |
| | Methyl-L- | | ((3-methyltetrahydrofuran-3-yl)methyl)glycine | D | |
| | leucine | | | | |
| C-4.4 | Boc-N- | IM-1.4 | N-(2-(7-Oxabicyclo[2.2.1]heptan-2-yl)ethyl)-N-(N- | 0.95 | 427.39 |
| | Methyl-L- | | (tert-butoxycarbonyl)-N-methyl-L-leucyl)glycine | D | |
| | leucine | | | | |
| C-4.5 | Boc-N- | IM-1.5 | N-(N-(tert-Butoxycarbonyl)-N-methyl-L-leucyl)-N- | 1.12 | 441.36 |
| | Methyl-L- | | (2-(5,5-dimethyltetrahydrofuran-2-yl)ethyl)glycine | D | |
| | leucine | | | | |
| C-4.6 | Boc-N- | IM-1.6 | N-(N-(tert-Butoxycarbonyl)-N-methyl-L-leucyl)-N- | 0.97 | 401.25 |
| | Methyl-L- | | (2-(tetrahydrofuran-2-yl)ethyl)glycine | D | |
| | leucine | | | | |
| C-4.7 | Boc-N- | IM-1.7 | N-(N-(tert-Butoxycarbonyl)-N-methyl-L-leucyl)-N- | 0.97 | 415.42 |
| | Methyl-L- | | (2-(tetrahydro-2H-pyran-3-yl)ethyl)glycine | D | |
| | leucine | | | | |
| C-4.8 | Boc-N- | IM-1.8 | N-(2-(2,9-Dioxaspiro[5.5]undecan-3-yl)ethyl)-N-(N- | 1.00 | 485.31 |
| | Methyl-L- | | (tert-butoxycarbonyl)-N-methyl-L-leucyl)glycine | D | |
| | leucine | | | | |
| C-4.9 | Boc-N- | IM-1.9 | N-(2-(1,4-Dioxan-2-yl)ethyl)-N-(N-(tert- | 0.88 | 403.4 |
| | Methyl-L- | | butoxycarbonyl)-N-methyl-L-leucyl)glycine | D | |
| | leucine | | | | |
| C-4.10 | Boc-N- | IM-1.10 | N-(N-(tert-Butoxycarbonyl)-N-methyl-L-leucyl)-N- | 1.05 | 415.43 |
| | Methyl-L- | | (2-(tetrahydro-2H-pyran-2-yl)ethyl)glycine | D | |
| | leucine | | | | |
| C-4.11 | Boc-N- | IM-1.11 | N-(N-(tert-Butoxycarbonyl)-N-methyl-L-leucyl)-N- | 0.54-0.58 | 415.25 |
| | Methyl-L- | | (2-(3-methoxycyclobutyl)ethyl)glycine | I | |
| | leucine | | | | |
| C-4.12 | D2-1.3 | IM-1.12 | N-(2-((tert-Butoxycarbonyl)(methyl)amino)pent-4- | 0.58 | 395.22 |
| | | | ynoyl)-N-(2-cyclohexylethyl)glycine | I | |

### General Method 5 for the Synthesis of Building Blocks C

### (S)-2-(2-((tert-Butoxycarbonyl)(methyl)amino)-N,4-dimethylpentanamido)-2,3-dihydro-1H-indene-2-carboxylic acid (C-5.1)

Step 1: HATU (1.24 g, 3.25 mmol) is added to a RT soln. of Boc-N-methyl-L-leucine (800 mg, 3.25 mmol), methyl 2-amino-indan-2-carboxylate HCl (764 mg, 3.25 mmol), and DIPEA (2.23 mL, 13 mmol) in DMF (8 mL). The RM is stirred at RT for 1 h, then diluted with water (20 mL) and DCM (50 mL). The layers are separated and the aq. layer is extracted with DCM (2×50 mL). The combined org. layers are washed with brine (20 mL), dried (MgSO₄), filtered, and concentrated. Purification by FC (eluting with 20% to 55% EtOAc in hept, R_{f}=0.34 in EtOAc/hept 3:7) yields methyl (S)-2-(2-((tert-butoxycarbonyl)amino)-4-methyl pentanamido)-2,3-dihydro-1H-indene-2-carboxylate (1.07 g, 79%) as a colourless oil. LC-MS B: t_{R}=1.07 min; [M+H]⁺=419.17.

Step 2: NaH (36.6 mg, 0.956 mmol) is added to a 0° C. soln. of methyl (S)-2-(2-((tert-butoxycarbonyl)amino)-4-methylpentanamido)-2,3-dihydro-1H-indene-2-carboxylate (200 mg, 0.478 mmol) in DMF (6 mL). After 10 min stirring, Mel (60 μL, 0.96 mmol) is added and the ice bath removed. The mix. is stirred at RT for 1 h, then the RM is partitioned between 2 N HCl (6 mL) and DCM (75 mL), and the layers are separated. The aq. layer is re-extracted with DCM (2×75 mL) and the combined org. layers are washed with brine (30 mL), dried (Na₂SO₄), filtered, and evaporated. The crude product is purified by FC (eluting with 35% to 80% EtOAc in hept) to give methyl (S)-2-(2-((tert-butoxycarbonyl)amino)-N,4-dimethylpentanamido)-2,3-dihydro-1H-indene-2-carboxylate as a colourless oil. LC-MS B: t_{R}=1.11 min; [M+H]⁺=433.07.

Step 3: 4 M NaOH soln. (21 mL, 83.2 mmol) is added to a RT soln. of methyl (S)-2-(2-((tert-butoxycarbonyl)amino)-N,4-dimethylpentanamido)-2,3-dihydro-1H-indene-2-carboxylate (90 mg, 2.08 mmol) in MeOH (30 mL), and the RM is stirred at 50° C. for 4 h. The RM is cooled to RT, then the mix. is diluted with DCM (100 mL) and acidified with a 2 M HCl soln. (10 mL). The layers are separated and the aq. layer is extracted with DCM (2×75 mL). The combined org. layers are washed with brine (30 mL), dried (MgSO₄), filtered, and concentrated to yield the title compound C-5.1 (796 mg, 91%) as a white foam which is used as such in the next step. LC-MS B: t_{R}=1.01 min; [M+H]⁺=419.09.

Listed in Table C-5 below are building blocks C that are prepared from the corresponding starting materials in analogy to the 3-step sequence described above for C-5.1.

TABLE C-5

| | | | | t_{R} [min] | MS-data |
|---|---|---|---|---|---|
| | | | | LC-MS | m/z |
| No. | SM A | SM B | Product | Method | [M + H]⁺ |
| C-5.2 | Boc-N-Methyl- | Methyl alpha- | (S)-2-(2-((tert- | 0.88 | 345.26 |
| | L-leucine | aminoisobutyrate | Butoxycarbonyl)(methyl)amino)- | B | |
| | | HCl | N,4-dimethylpentanamido)-2- | | |
| | | | methylpropanoic acid | | |
| C-5.3 | Boc-N-Methyl- | Methyl 2-amino-2- | 2-((S)-2-((tert- | 0.91 | 357.32 |
| | L-leucine | cyclopropylacetate | Butoxycarbonyl)(methyl)amino)- | B | |
| | | (HCl salt) | N,4-dimethylpentanamido)-2- | | |
| | | | cyclopropylacetic acid | | |
| C-5.4 | Boc-N-Methyl- | Methyl 1-amino-3,3- | (S)-1-(2-((tert- | 0.95 | 393.30 |
| | L-leucine | difluorocyclobutane- | Butoxycarbonyl)(methyl)amino)- | B | |
| | | 1-carboxylate (HCl | N,4-dimethylpentanamido)-3,3- | | |
| | | salt) | difluorocyclobutane-1-carboxylic | | |
| | | | acid | | |

### General Method 6 for the Synthesis of Building Blocks C

### (R)-1-(N-(tert-Butoxycarbonyl)-N-methyl-L-leucyl)-4-(3-hydroxypropyl)piperazine-2-carboxylic acid HCl salt (C-6.1)

Step 1: K₂CO₃ (1.66 g, 12 mmol) is added to a RT soln. of 1-(tert-butyl) 2-methyl (R)-piperazine-1,2-dicarboxylate (1.0 g, 4.01 mmol) and benzyl 3-bromopropyl ether (0.95 mL, 5.22 mmol) in MeCN (10 mL) and the resulting mix. is stirred at 60° C. for 17 h. Water (20 mL) and DCM (75 mL) are added to the RM, then the two layers are separated and the aq. layer is extracted with DCM (2×50 mL). The combined org. extracts are washed with brine (50 mL), dried (MgSO₄), filtered, and concentrated. Purification by FC (eluting with 5% to 25% EtOAc in hept) yields (R)-4-(3-benzyloxy-propyl)-piperazine-1,2-dicarboxylic acid 1-tert-butyl ester 2-methyl ester (1.58 g, 100%) as a colourless oil. LC-MS B: t_{R}=0.75 min; [M+H]⁺=393.43.

Step 2: 4 M HCl in dioxane (5 mL, 20 mmol) is added to a RT soln. of (R)-4-(3-benzyloxy-propyl)-piperazine-1,2-dicarboxylic acid 1-tert-butyl ester 2-methyl ester (1.57 g, 4 mmol) in dioxane (7 mL) and the resulting mix. is stirred at 50° C. for 2 h. The RM is cooled to RT, then diluted with DCM (100 mL) and sat. aq. K₂CO₃ (20 mL) is added. The layers are separated and the aq. layer is extracted with DCM (2×75 mL). The combined org. layers are washed with brine (100 mL), dried (MgSO₄), filtered, and concentrated to give (R)-4-(3-benzyloxy-propyl)-piperazine-2-carboxylic acid methyl ester (1.06 g, 91%) as colourless oil which is used as such in the next step. LC-MS B: t_{R}=0.53 min; [M+H]⁺=293.32.

Step 3: HATU (1.36 g, 3.58 mmol) is added to a RT soln. of Boc-N-methyl-L-leucine (880 mg, 3.58 mmol), (R)-4-(3-benzyloxy-propyl)-piperazine-2-carboxylic acid methyl ester (1.05 g, 3.58 mmol), and DIPEA (1.84 mL, 10.7 mmol) in DMF (11 mL). The resulting mix. is stirred at RT for 1 h, the RM is diluted with DCM (100 mL) and water (10 mL). The layers are separated and the aq. layer is extracted with DCM (2×75 mL). The combined org. layers are washed with brine (50 mL), dried (MgSO₄), filtered, and concentrated. Purification by FC (eluting with 15% to 42% EtOAc in hept) yields methyl (R)-4-(3-(benzyloxy)propyl)-1-(N-(tert-butoxycarbonyl)-N-methyl-L-leucyl)piperazine-2-carboxylate (1.67 g, 90%) as a colourless oil. LC-MS B: t_{R}=0.90 min; [M+H]⁺=520.39.

Step 4: 2 M NaOH soln. (32 mL, 63.9 mmol) is added to a RT soln. of methyl (R)-4-(3-(benzyloxy)propyl)-1-(N-(tert-butoxycarbonyl)-N-methyl-L-leucyl)piperazine-2-carboxylate (1.66 g, 3.19 mmol) in MeOH (60 mL) and the RM is stirred at 50° C. for 1 h. The RM is cooled to RT and the RM is diluted with DCM (100 mL) and acidified with a 25% aq. HCl soln. (10 mL). The layers are separated and the aq. layer is extracted with DCM (2×75 mL). The combined org. layers are washed with brine (100 mL), dried (MgSO₄), filtered, and concentrated to yield (R)-4-(3-benzyloxy-propyl)-1-[(S)-2-(tert-butoxycarbonyl-methyl-amino)-4-methyl-pentanoyl]-piperazine-2-carboxylic acid (1.47 g, 85%) as a white solid which is used as such in the next step. LC-MS B: t_{R}=0.84 min; [M+H]⁺=506.36.

Step 5: A soln. of (R)-4-(3-benzyloxy-propyl)-1-[(S)-2-(tert-butoxycarbonyl-methyl-amino)-4-methyl-pentanoyl]-piperazine-2-carboxylic acid (1.74 g, 3.21 mmol) in EtOH (20 mL) is inertised with N₂/vacuum (3×) before 10% Pd/C (171 mg, 0.16 mmol) is added. After inertising another three times a H₂ balloon is connected and the RM is stirred at RT for 18 h. The mix. is concentrated and filtered over a celite plug rinsing with EtOH. The filtrate is concentrated to give the title compound C-6.1 (1.14 g, 79%) as a white solid. LC-MS B: t_{R}=0.67 min; [M+H]⁺=416.34.

### (R)-1-(N-(tert-Butoxycarbonyl)-N-methyl-L-leucyl)-4-(2-hydroxyethyl)piperazine-2-carboxylic acid HCl-salt (C-6.2)

The title compound is prepared following the 5-step sequence described for C-6.1, using in step 1 benzyl 2-bromoethyl ether instead of benzyl 3-bromopropyl ether. LC-MS B: t_{R}=0.67 min; [M+H]⁺=402.07.

### (R)-1-(N-(tert-Butoxycarbonyl)-N-methyl-L-leucyl)-4-(2-methoxyethyl)piperazine-2-carboxylic acid HCl salt (C-6.3)

Step 1: 1-Bromo-2-methoxyethane (4.31 mL, 44.9 mmol) is added to a RT soln. of 1-(tert-butyl) 2-methyl (R)-piperazine-1,2-dicarboxylate (4.00 g, 16 mmol) and DIPEA (8.41 mL, 48.1 mmol) in MeCN (87 mL). The resulting mix. is stirred at 80° C. for 18 h. The mix. is concentrated, and the residue partitioned between water (10 mL) and DCM (50 mL). The layers are separated, and the aq. layer re-extracted with DCM (2×75 mL). The combined org. extracts are washed with brine (50 mL), dried (MgSO₄), filtered, and evaporated to give (R)-4-(2-methoxy-ethyl)-piperazine-1,2-dicarboxylic acid 1-tert-butyl ester 2-methyl ester as an orange oil. LC-MS B: t_{R}=0.54 min; [M+H]⁺=303.36.

Steps 2 to 4: The title compound is prepared from (R)-4-(2-methoxy-ethyl)-piperazine-1,2-dicarboxylic acid 1-tert-butyl ester 2-methyl ester following the sequence of reactions described for C-6.1, steps 2 to 4. LC-MS B: t_{R}=0.71 min; [M+H]⁺=416.36.

### (S)-1-(2-((tert-Butoxycarbonyl)(methyl)amino)-N-(3-methoxypropyl)-4-methylpentanamido)cyclopropane-1-carboxylic acid (C-6.4)

Step 1: 1-(3-Methoxy-propylamino)-cyclopropanecarboxylic acid methyl ester is prepared from methyl 1-aminocyclopropanecarboxylate and 1-bromo-3-methoxypropane following the reaction described for C-6.3, step 1. LC-MS B: t_{R}=0.39 min; [M+H]⁺=188.43.

Steps 2&3: The title compound is prepared from 1-(3-methoxy-propylamino)-cyclopropanecarboxylic acid methyl ester following the sequence of reactions described for C-6.1, steps 3&4. LC-MS B: t_{R}=0.91 min; [M+H]⁺=401.30.

### General Method 7 for the Synthesis of Building Blocks C

### (R)-1-(N-(tert-Butoxycarbonyl)-N-methyl-L-leucyl)-4-((2,2,2-trichloroethoxy)carbonyl)piperazine-2-carboxylic acid (C-7.1)

Step 1: 2,2,2-Trichloroethyl chloroformate (1.2 mL, 8.54 mmol) is added to a RT soln. of 1-(tert-butyl) 2-methyl (R)-piperazine-1,2-dicarboxylate (2.0 g, 8.02 mmol) and DIPEA (2.88 mL, 16.5 mmol) in DCM (40 mL). The resulting mix. is stirred at RT for 45 min. The RM is diluted with DCM (100 mL) and water (20 mL). The layers are separated and the aq. layer is extracted with DCM (2×75 mL). The combined org. layers are washed with brine (50 mL), dried (MgSO₄), filtered, and concentrated. Purification by FC (eluting with 20% to 60% EtOAc in hept, R_{f}=0.3 in EtOAc/hept 1:1) yields 1-(tert-butyl) 2-methyl 4-(2,2,2-trichloroethyl) (R)-piperazine-1,2,4-tricarboxylate (2.90 g, 74%) as a white solid. LC-MS B: t_{R}=1.13 min; [M+H]⁺=545.75.

Steps 2 to 4: The title compound is prepared from 1-(tert-butyl) 2-methyl 4-(2,2,2-trichloroethyl) (R)-piperazine-1,2,4-tricarboxylate following the sequence of reactions described for C-6.1, steps 2 to 4. LC-MS B: t_{R}=1.03 min; [M+H]⁺=531.98.

### General Method 8 for the Synthesis of Building Blocks C

### (R)-1-(N-(tert-Butoxycarbonyl)-N-methyl-L-leucyl)-4-phenylpiperazine-2-carboxylic acid (C-8.1)

Step 1: Cu(OAc)₂ (749 mg, 6.2 mmol) is added to a RT soln. of 1-(tert-butyl) 2-methyl (R)-piperazine-1,2-dicarboxylate (1.00 g, 4.01 mmol) and phenylboronic acid (749 mg, 4.01 mmol) in DCM (20 mL) and the resulting mix. is stirred at RT overnight. The RM is diluted with DCM and washed with cold water (20 mL), and brine (20 mL). The org. layer is dried (MgSO₄), filtered, and evaporated. Purification by prep. HPLC (basic) yields (R)-4-phenyl-piperazine-1,2-dicarboxylic acid 1-tert-butyl ester 2-methyl ester (337 mg, 26%) as a colourless oil. LC-MS B: t_{R}=1.02 min; [M+H]⁺=321.13.

Step 2: TFA (0.8 mL, 10.6 mmol) is added to a soln. of (R)-4-phenyl-piperazine-1,2-dicarboxylic acid 1-tert-butyl ester 2-methyl ester (337 mg, 1.06 mmol) in DCM (40 mL) and the resulting mix. is stirred for 24 h. The RM is diluted with DCM (10 mL) and neutralised with a sat. aq. soln. of NaHCO₃ (30 mL). The layers are separated, and the aq. layer re-extracted with DCM (20 mL). The combined org. extracts are dried (Na₂SO₄), filtered, and concentrated to give (R)-4-phenyl-piperazine-2-carboxylic acid methyl ester (232 mg, 100%) as a yellowish oil. LC-MS B: t_{R}=0.52 min; [M+H]⁺=221.32.

Steps 3&4: The title compound is prepared from Boc-N-methyl-L-leucine and (R)-4-phenyl-piperazine-2-carboxylic acid methyl ester following the sequence of reactions described for C-2.1. LC-MS B: t_{R}=1.03 min; [M+H]⁺=433.9.

### (RS)-1-(N-(tert-Butoxycarbonyl)-N-methyl-L-leucyl)-4-(5-fluoropyridin-2-yl)piperazine-2-carboxylic acid (C-8.2)

Step 1: A mixture of 1-tert-butyl 2-methyl (2R)-piperazine-1,2-dicarboxylate (2500 mg, 10 mmol), 2-bromo-5-fluoropyridine (2161 mg, 12 mmol), tris(dibenzylideneacetone)dipalladium(0) (473 mg, 0.501 mmol) and 1,3-bis(2,6-diisopropylphenyl)imidazolium chloride (426 mg, 1 mmol) in toluene (30 mL) is degassed and inertised with Argon. The RM is then heated up to 100° C. for 4h30 to reach complete conversion as monitored by LC-MS. The solution is cooled back to RT and is filtered over a glass fiber filter. Water is added to the resulting filtrate and the org. layer is collected; the inorg. phase is then further extracted with EtOAc (2×). The combined organic phase is successively washed with sat. aq. NH₄Cl, sat. aq. NaHCO₃ and brine, dried over MgSO₄ then concentrated under reduced pressure. The crude is purified by FC (0% to 30% EtOAc in Hept) to yield 1-(tert-butyl) 2-methyl (R)-4-(5-fluoropyridin-2-yl)piperazine-1,2-dicarboxylate as a yellow oil (2.21 g). LC-MS B: t_{R}=0.92 min; [M+H]⁺=340.16.

Step 2-4: The title compound is prepared from boc-N-methyl-L-leucine and 1-(tert-butyl) 2-methyl (R)-4-(5-fluoropyridin-2-yl)piperazine-1,2-dicarboxylate following the sequence of reactions 2 to 4 described for C-8.1. Extensive epimerization is observed at the piperazine chiral center at the end of the 4 step sequence. LC-MS B: t_{R}=0.96 min; [M+H]⁺=453.37.

### General Method 9 for the Synthesis of Building Blocks C

### (R)-1-(N-(tert-Butoxycarbonyl)-N-methyl-L-leucyl)-4-(pyridin-3-ylsulfonyl)piperazine-2-carboxylic acid (C-9.1)

Step 1: Pyridine-3-sulfonyl chloride (2.33 g, 12.8 mmol) is added to a RT soln. of 1-(tert-butyl) 2-methyl (R)-piperazine-1,2-dicarboxylate (2.00 g, 8.02 mmol) and TEA (3.37 mL, 24.1 mmol) in DCM (80 mL) and the resulting mix. is stirred at RT for 1 h. The RM is diluted with DCM (75 mL) and water (10 mL). The layers are separated and the aq. layer is extracted with DCM (2×75 mL). The combined org. layers are washed with brine (50 mL), dried (MgSO₄), filtered, and concentrated. Purification by FC (eluting with 80% to 85% EtOAc in hept, R_{f}=0.42 in EtOAc/hept 7:3) yields (R)-4-(pyridine-3-sulfonyl)-piperazine-1,2-dicarboxylic acid 1-tert-butyl ester 2-methyl ester (3.06 g, 99%) as a white foam. LC-MS B: t_{R}=0.89 min; [M+H]⁺=386.16.

Step 2: 4 M HCl in dioxane (9.73 mL, 38.9 mmol) is added to a RT soln. of (R)-4-(pyridine-3-sulfonyl)-piperazine-1,2-dicarboxylic acid 1-tert-butyl ester 2-methyl ester (3.0 g, 7.78 mmol) in dioxane (10 mL). The resulting mix. is stirred at 50° C. for 2 h. The suspension is filtered and the solids are rinsed with TBME (20 mL) to give methyl (R)-4-(pyridin-3-ylsulfonyl)piperazine-2-carboxylate dihydrochloride (3.12 g, 112%) as a white solid. LC-MS B: t_{R}=0.44 min; [M+H]⁺=286.15.

Steps 3&4: The title compound is prepared from Boc-N-methyl-L-leucine and (methyl (R)-4-(pyridin-3-ylsulfonyl)piperazine-2-carboxylate dihydrochloride following the sequence of reactions described for C-2.1. LC-MS B: t_{R}=0.93 min; [M+H]⁺=499.27.

### (R)-1-(N-(tert-Butoxycarbonyl)-N-methyl-L-leucyl)-4-((5-methoxypyridin-3-yl)sulfonyl)piperazine-2-carboxylic acid (C-9.2)

The title compound is prepared following the 4-step sequence described for C-9.1 using in step 1, 5-methoxy-pyridine-3-sulfonyl chloride instead of pyridine-3-sulfonyl chloride. LC-MS B: t_{R}=0.96 min; [M+H]⁺=529.10.

### General Method 10 for the Synthesis of Building Blocks C

Listed in Table C-10 below are building blocks C that are prepared from the corresponding starting materials in analogy to the sequence described above for C-1.1, steps 3&4.

TABLE C-10

| | | | | t_{R} [min] | MS-data |
|---|---|---|---|---|---|
| | | | | LC-MS | m/z |
| No. | SM A | SM B | Product | Method | [M + H]⁺ |
| C-10.1 | D2-1.1 | IM- | N-((S)-2-((tert-Butoxycarbonyl)(methyl)amino)-3- | 0.55-0.62 | 427.25 |
| | | 2.2 | cyclopentylpropanoyl)-N-(((R)-tetrahydro-2H-pyran- | I | |
| | | | 2-yl)methyl)glycine | | |
| C-10.2 | D2-1.1 | IM- | N-((S)-2-((tert-Butoxycarbonyl)(methyl)amino)-3- | 0.59-0.61 | 427.24 |
| | | 2.1 | cyclopentylpropanoyl)-N-(((S)-tetrahydro-2H-pyran- | I | |
| | | | 2-yl)methyl)glycine | | |
| C-10.3 | D2-1.2 | IM- | N-((S)-2-((tert-Butoxycarbonyl)(methyl)amino)-3- | 0.58-0.66 | 441.28 |
| | | 2.2 | cyclohexylpropanoyl)-N-(((R)-tetrahydro-2H-pyran-2- | I | |
| | | | yl)methyl)glycine | | |
| C-10.4 | D2-1.2 | IM- | N-((S)-2-((tert-Butoxycarbonyl)(methyl)amino)-3- | 0.59-0.64 | 441.27 |
| | | 2.1 | cyclohexylpropanoyl)-N-(((S)-tetrahydro-2H-pyran-2- | I | |
| | | | yl)methyl)glycine | | |
| C-10.5 | D2-2.1 | IM- | N-((S)-2-((tert-Butoxycarbonyl)(methyl)amino)-3- | 0.55-0.59 | 413.22 |
| | | 2.1 | cyclobutylpropanoyl)-N-(((S)-tetrahydro-2H-pyran-2- | I | |
| | | | yl)methyl)glycine | | |
| C-10.6 | D2-2.1 | IM- | N-((S)-2-((tert-Butoxycarbonyl)(methyl)amino)-3- | 0.53-0.60 | 413.20 |
| | | 2.2 | cyclobutylpropanoyl)-N-(((R)-tetrahydro-2H-pyran-2- | I | |
| | | | yl)methyl)glycine | | |
| C-10.7 | D2-1.4 | IM- | N-((S)-2-((tert-Butoxycarbonyl)(methyl)amino)-3- | 0.48-0.53 | 399.21 |
| | | 2.2 | cyclopropylpropanoyl)-N-(((R)-tetrahydro-2H-pyran- | I | |
| | | | 2-yl)methyl)glycine | | |
| C-10.8 | D2-1.4 | IM- | N-((S)-2-((tert-Butoxycarbonyl)(methyl)amino)-3- | 0.51-0.56 | 413.3 |
| | | 2.3 | cyclopropylpropanoyl)-N-(2-((S)-tetrahydro-2H- | I | |
| | | | pyran-2-yl)ethyl)glycine | | |
| C-10.9 | D2-1.4 | IM- | N-((S)-2-((tert-Butoxycarbonyl)(methyl)amino)-3- | 0.52-0.58 | 413.23 |
| | | 2.4 | cyclopropylpropanoyl)-N-(2-((R)-tetrahydro-2H- | I | |
| | | | pyran-2-yl)ethyl)glycine | | |
| C-10.10 | D2-2.1 | IM- | N-((S)-2-((tert-Butoxycarbonyl)(methyl)amino)-3- | 0.55-0.60 | 427.34 |
| | | 2.3 | cyclobutylpropanoyl)-N-(2-((S)-tetrahydro-2H-pyran- | I | |
| | | | 2-yl)ethyl)glycine | | |
| C-10.11 | D2-2.1 | IM- | N-((S)-2-((tert-Butoxycarbonyl)(methyl)amino)-3- | 0.55-0.60 | 427.33 |
| | | 2.4 | cyclobutylpropanoyl)-N-(2-((R)-tetrahydro-2H-pyran- | I | |
| | | | 2-yl)ethyl)glycine | | |
| C-10.12 | Boc-N- | IM- | N-(N-(tert-Butoxycarbonyl)-N-methyl-L-leucyl)-N-(2- | 0.53-0.61 | 415.25 |
| | Methyl-L- | 2.3 | ((S)-tetrahydro-2H-pyran-2-yl)ethyl)glycine | I | |
| | leucine | | | | |
| C-10.13 | Boc-N- | IM- | N-(N-(tert-Butoxycarbonyl)-N-methyl-L-leucyl)-N-(2- | 0.53-0.61 | 415.25 |
| | Methyl-L- | 2.4 | ((R)-tetrahydro-2H-pyran-2-yl)ethyl)glycine | I | |
| | leucine | | | | |
| C-10.14 | D2-1.1 | IM- | N-((S)-2-((tert-Butoxycarbonyl)(methyl)amino)-3- | 1.01 | 441.39 |
| | | 1.10 | cyclopentylpropanoyl)-N-(2-(tetrahydro-2H-pyran-2- | B | |
| | | | yl)ethyl)glycine | | |
| C-10.15 | D2-1.2 | IM- | N-((S)-2-((tert-Butoxycarbonyl)(methyl)amino)-3- | 0.61-0.65 | 455.31 |
| | | 1.10 | cyclohexylpropanoyl)-N-(2-(tetrahydro-2H-pyran-2- | I | |
| | | | yl)ethyl)glycine | | |
| C-10.16 | D2-1.1 | IM- | (S)-N-(2-((tert-Butoxycarbonyl)(methyl)amino)-3- | 0.70 | 439.29 |
| | | 1.12 | cyclopentylpropanoyl)-N-(2-cyclohexylethyl)glycine | I | |
| C-10.17 | D2-1.2 | IM- | (S)-N-(2-((tert-Butoxycarbonyl)(methyl)amino)-3- | 0.73 | 453.34 |
| | | 1.12 | cyclohexylpropanoyl)-N-(2-cyclohexylethyl)glycine | I | |
| C-10.18 | D2-2.1 | IM- | (S)-N-(2-((tert-Butoxycarbonyl)(methyl)amino)-3- | 0.67 | 425.28 |
| | | 1.12 | cyclobutylpropanoyl)-N-(2-cyclohexylethyl)glycine | I | |
| C-10.19 | Boc-N- | IM- | N-(N-(tert-Butoxycarbonyl)-N-methyl-L-leucyl)-N-(2- | 0.72 | 413.26 |
| | Methyl-L- | 1.12 | cyclohexylethyl) glycine | I | |
| | leucine | | | | |
| C-10.20 | Boc-N- | IM- | N-(N-(tert-Butoxycarbonyl)-N-methyl-L-leucyl)-N- | 0.51-0.59 | 401.89 |
| | Methyl-L- | 2.2 | (((R)-tetrahydro-2H-pyran-2-yl)methyl)glycine | I | |
| | leucine | | | | |
| C-10.21 | Boc-N- | IM- | N-(N-(tert-Butoxycarbonyl)-N-methyl-L-leucyl)-N- | 0.54 | 401.87 |
| | Methyl-L- | 2.1 | (((S)-tetrahydro-2H-pyran-2-yl)methyl)glycine | I | |
| | leucine | | | | |
| C-10.22 | Boc-N- | IM- | N-(N-(tert-Butoxycarbonyl)-N-methyl-L-leucyl)-N-(2- | 1.05 | 415.43 |
| | Methyl-L- | 1.10 | (tetrahydro-2H-pyran-2-yl)ethyl)glycine | B | |
| | leucine | | | | |
| C.10.23 | Boc-N- | IM- | N-(N-(tert-Butoxycarbonyl)-N-methylglycyl)-N-(2- | 2.09 | No |
| | methyl- | 1.12 | cyclohexylethyl)glycine | F | ionisation |
| | glycine | | | | |
| C-10.24 | Boc-N- | IM- | N-(N-(tert-Butoxycarbonyl)-N-methyl-L-alanyl)-N-(2- | 2.20 | 371.20 |
| | methyl-L- | 1.12 | cyclohexylethyl)glycine | F | |
| | alanine | | | | |
| C-10.25 | Boc-N- | IM- | N-(N-(tert-Butoxycarbonyl)-N-methyl-L-leucyl)-N-(3- | 1.78-1.84 | 421.20 |
| | methyl-L- | 1.15 | phenylpropyl)glycine | J | |
| | leucine | | | | |
| C-10.26 | D2-1.4 | IM- | (S)-N-(2-((tert-Butoxycarbonyl)(methyl)amino)-3- | 0.63 | 411.28 |
| | | 1.12 | cyclopropylpropanoyl)-N-(2-cyclohexylethyl)glycine | I | |

### General Method 11 for the Synthesis of Building Blocks C

### (S)-1-(2-((tert-Butoxycarbonyl)(methyl)amino)-3-cyclopentyl-N-methylpropanamido)cyclopropane-1-carboxylic acid (C-11.1)

Step 1: Benzyl chloroformate (4.6 mL, 30.6 mmol) is added to a RT soln. of methyl 1-aminocyclopropanecarboxylate (3.25 g, 27.7 mmol) and NaHCO₃ (9.64 g, 115 mmol) in DCM (30 mL) and H₂O (30 mL). The resulting mix. is stirred at RT. After 2 h, the two layers are separated, and the aq. layer extracted with DCM (2×30 mL). The combined org. extracts are concentrated to obtain 1-benzyloxycarbonylamino-cyclopropanecarboxylic acid methyl ester (7.28 g, 106%) as a yellow solid. LC-MS B: t_{R}=0.79 min; [M+H]⁺=250.37.

Step 2: NaH (1.85 g, 46.3 mmol) is added to a 0° C. soln. of DMF (45 mL), followed by a soln. of 1-benzyloxycarbonylamino-cyclopropanecarboxylic acid methyl ester (7.28 g, 29.2 mmol) in DMF (30 mL). After 45 min, Mel (5.5 mL, 87.5 mmol) is added and the mix. is warmed to RT overnight. The RM is partitioned between H₂O (50 mL) and EtOAc (150 mL) and the layers are separated. The aq. layer is re-extracted with EtOAc (2×75 mL) and the combined org. layers are washed with brine (40 mL), dried (MgSO₄), filtered, and evaporated to obtain 1-(benzyloxycarbonyl-methyl-amino)-cyclopropanecarboxylic acid methyl ester (7.70 g, 100%) as an orange liquid which is used as such in the next step. LC-MS B: t_{R}=0.88 min; [M+H]⁺=264.33.

Step 3: A soln. of 1-(benzyloxycarbonyl-methyl-amino)-cyclopropanecarboxylic acid methyl ester (4.00 g, 11.4 mmol) in MeOH (18 mL) is inertised with N₂/vacuum (3×) before 10% Pd/C (606 mg, 0.57 mmol) is added. After inertising another three times a H₂ balloon is connected and the RM is stirred for 18 h. The mix. is concentrated and filtered over a celite plug rinsing with EtOH. The filtrate is concentrated (careful, product is volatile) to give 1-methylamino-cyclopropanecarboxylic acid methyl ester (653 mg, 44%). LC-MS B: t_{R}=0.26 min; [M+H]⁺=130.24.

Steps 4&5: The title compound is prepared from D2-1.1 and 1-methylamino-cyclopropanecarboxylic acid methyl ester, following the 2-step sequence described for C-2.1. LC-MS B: t_{R}=0.92 min; [M+H]⁺=369.29.

General method 12 for the synthesis of building blocks C N—(N-(tert-Butoxycarbonyl)-N-methyl-L-leucyl)-N-(3-methoxypropyl)-D-alanine (C-12.1)

Step 1: NaH₃BCN (398 mg, 6.02 mmol) is added to a RT soln. of D-alanine methyl ester HCl (600 mg, 4.3 mmol), 3-methoxy-propionaldehyde (429 mg, 4.73 mmol), molecular sieves 3A (450 mg), and AcOH (0.295 mL, 5.16 mmol) in MeOH (19 mL). The resulting mix. is stirred at RT for 35 min. The mix. is concentrated and to the residue is added DCM. The org. layer is washed with sat. NaHCO₃ and the aq. layer is re-extracted with DCM. The combined org. extracts are washed with brine, dried (MgSO₄), filtered, and concentrated to yield (R)-2-(3-methoxy-propylamino)-propionic acid methyl ester (615 mg, 82%) as a colourless oil. LC-MS B: t_{R}=0.33; [M+H]⁺=176.50.

Steps 2&3: The title compound is prepared from (R)-2-(3-methoxy-propylamino)-propionic acid methyl ester following the 2-step sequence described for C-2.1. LC-MS B: t_{R}=0.91 min; [M+H]⁺=389.42.

### General Method 13 for the Synthesis of Building Blocks C

### (S)-1-(2-((tert-Butoxycarbonyl)(methyl)amino)-N-(2-methoxyethyl)-4-methylpentanamido)cyclopropane-1-carboxylic acid (C-13.1)

Step 1: Benzyl bromide (4.41 mL, 36.4 mmol) is added to a suspension of 1-tert-butoxycarbonylamino-cyclopropanecarboxylic acid (7.00 g, 33.0 mmol) and K₂CO₃ (6.92 g, 49.6 mmol) in MeCN (320 mL). The resulting mix. is heated to 60° C. for 15 h. The RM is concentrated and to the residue is added EtOAc and water. The org. layer is separated and washed with brine, dried (MgSO₄), filtered, and concentrated to yield 1-tert-butoxycarbonylamino-cyclopropanecarboxylic acid benzyl ester (9.97 g, 104%) as a white solid. LC-MS B: t_{R}=0.95 min; [M+H]⁺=292.31.

Step 2: 4 M HCl in dioxane (84.0 mL, 336 mmol) is added to a RT suspension of 1-tert-butoxycarbonylamino-cyclopropanecarboxylic acid benzyl ester (9.97 g, 33.0 mmol) in DCM (20 mL) and the resulting mix. stirred at RT for 1.5 h. The RM is concentrated and co-evaporated with DCM at HV to obtain 1-amino-cyclopropanecarboxylic acid benzyl ester HCl (7.93 g, 105%) as a white solid. LC-MS B: t_{R}=0.50 min; [M+H]⁺=192.34.

Step 3: 2-Nitrobenzenesulfonyl chloride (8.30 g, 36.4 mmol) is added portionwise to a 0° C. suspension of 1-amino-cyclopropanecarboxylic acid benzyl ester HCl (7.93 g, 33.0 mmol) and TEA (13.9 mL, 99.1 mmol) in DCM (70 mL). The ice bath is removed after 30 min and the mix. is stirred at RT for 1.5 h. The RM is partitioned between sat. NaHCO₃ and DCM and the layers are separated. The aq. layer is re-extracted with DCM and the combined org. layers are washed with brine (40 mL), dried (MgSO₄), filtered, and concentrated. Purification by FC (EtOAc/hept 3:7 to 1:1, R_{f}=0.21 in EtOAc/hept 3:7) yields 1-(2-nitro-benzenesulfonylamino)-cyclopropanecarboxylic acid benzyl ester (12.05 g, 97%) as an orange oil. LC-MS B: t_{R}=0.96 min; [M+H]⁺=377.29.

Step 4: DIAD (2.1 mL, 10.2 mmol) is added dropwise to a 0° C. soln. of 1-(2-nitro-benzenesulfonylamino)-cyclopropanecarboxylic acid benzyl ester (2.00 g, 5.31 mmol), 2-methoxy-ethanol (0.635 mL, 7.97 mmol), and PPh₃ (2.83 g, 10.2 mmol) in THF (55 mL). The resulting mix. is stirred at 0° C. for 5 min then at RT for 3 h. The mix. is concentrated and purified by FC (EtOAc/hept, 3:7 to 1:1, R_{f}=0.39 in EtOAc/hept 1:1) to yield benzyl 1-((N-(2-methoxyethyl)-2-nitrophenyl)sulfonamido)cyclopropane-1-carboxylate (3.55 g, 154%) as a yellow oil. LC-MS B: t_{R}=1.03 min; [M+H]⁺=435.20.

Step 5: Thiophenol (0.844 mL, 7.97 mmol) is added dropwise to a RT soln. of benzyl 1-((N-(2-methoxyethyl)-2-nitrophenyl)sulfonamido)cyclopropane-1-carboxylate (3.55 g, 5.31 mmol) and K₂CO₃ (1.18 g, 8.5 mmol) in DMF (28 mL). The resulting mix. is stirred at RT for 2 h. The mix. is partitioned between EtOAc and water. The org. layer is separated, dried (MgSO₄), filtered, and concentrated until only DMF is present. Purification by prep. HPLC (basic) yields 1-(2-methoxy-ethylamino)-cyclopropanecarboxylic acid benzyl ester (848 mg, 64%) as a colourless oil. LC-MS B: t_{R}=0.58 min; [M+H]⁺=250.33.

Steps 6&7: The title compound is prepared from 1-(2-methoxy-ethylamino)-cyclopropanecarboxylic acid benzyl ester following the sequence of reactions described for C-1.1, steps 3&4. LC-MS B: t_{R}=0.91 min; [M+H]⁺=389.42.

### General Method 14 for the Synthesis of Building Blocks C

### (R)-1-(N-(tert-Butoxycarbonyl)-N-ethyl-L-leucyl)piperidine-2-carboxylic acid (C-14.1)

Step 1: Acetaldehyde (2.33 mL, 41.2 mmol) is added to a RT soln. of Fmoc-Leu-OH (3.00 g, 8.23 mmol), and pTsOH·H₂O (145 mg, 0.82 mmol) in PhMe (150 mL) and the resulting mix. is refluxed with a Dean Stark apparatus for 19 h. Acetaldehyde (2.33 mL, 41.2 mmol) is added again after 4 h and 6 h. The mix. is cooled to RT before being partitioned between EtOAc and sat. aq. NaHCO₃. The phases are separated and the aq. phase is re-extracted with EtOAc (2×). The combined org. layers are dried (Na₂SO₄), filtered, and concentrated. Purification by FC (eluting with 5% to 20% EtOAc in hept) yields (9H-fluoren-9-yl)methyl (4S)-4-isobutyl-(2R,S)-2-methyl-5-oxooxazolidine-3-carboxylate as a yellow oil. LC-MS B: t_{R}=1.13 min; [M+H]⁺=380.15.

Step 2: TFA (31.3 mL, 0.40 mol) followed by triethylsilane (3.89 mL, 23.9 mmol) are added to a RT soln. of (9H-fluoren-9-yl)methyl (4S)-4-isobutyl-(2R, S)-2-methyl-5-oxooxazolidine-3-carboxylate (3.0 g, 8.0 mmol) in DCM (30 mL) and the resulting mix. is stirred for 19 h. The mix. is concentrated and co-evaporated with DCM (2×). Purification by FC (eluting with 50% EtOAc in hept & 1% AcOH) yields N-(((9H-fluoren-9-yl)methoxy)carbonyl)-N-ethyl-L-leucine as a yellow oil. LC-MS B: t_{R}=1.05 min; [M+H]⁺=382.16.

Step 3: Methyl (R)-1-(N-(((9H-fluoren-9-yl)methoxy)carbonyl)-N-ethyl-L-leucyl)piperidine-2-carboxylate is prepared from N-(((9H-fluoren-9-yl)methoxy)carbonyl)-N-ethyl-L-leucine and (R)-piperidine-2-carboxylic acid methyl ester HCl-salt following the procedure described for C-2.1, step 1. LC-MS I: t_{R}=1.36 min; [M+H]⁺=507.33.

Step 4: A RT soln. of methyl (R)-1-(N-(((9H-fluoren-9-yl)methoxy)carbonyl)-N-ethyl-L-leucyl)piperidine-2-carboxylate (1.98 g, 3.91 mmol) and 2 M aq. NaOH (3.9 mL, 7.82 mmol) in MeOH (7.8 mL) is stirred for 18 h. The MeOH is evaporated in vacuo and the residue diluted with water and extracted with EtOAc (3×). The combined org. extracts are discarded and the aq. phase is evaporated to dryness to give (R)-1-(ethyl-L-leucyl)piperidine-2-carboxylic acid as a white solid. LC-MS B: t_{R}=0.55 min; [M+H]⁺=271.30.

Step 5: A soln. of Boc anhydride (898 mg, 4.12 mmol) in DMF (3.5 mL) is added to a RT soln. of (R)-1-(ethyl-L-leucyl)piperidine-2-carboxylic acid (1.06 g, 3.92 mmol) in DMF (3.5 mL) and 1 M aq. NaOH (3.9 mL, 3.92 mmol). After 19 h TEA (1.09 mL, 7.84 mmol) is added and after another 5 h additional Boc anhydride (170 mg, 0.78 mmol) is added and stirring is continued for 1 h. The RM is acidified with 2 M aq. HCl and extracted with TBME (2×). The combined org. extracts are washed with water, dried (Na₂SO₄), filtered, and concentrated. Purification by prep. HPLC (acidic) gives the title compound C-14.1 as a colourless oil. LC-MS B: t_{R}=0.98 min; [M+H]⁺=371.21.

### General Method 16 for the Synthesis of Building Blocks C

### N—(N-((Allyloxy)carbonyl)-N-methyl-L-leucyl)-N-(2-cyclohexylethyl)glycine (C-16.1)

Steps 1&2: The title compound is prepared from D2-3.1 and IM-1.12 following the procedure described for C-4.1, steps 1&2. LC-MS I: t_{R}=0.64 min; [M+H]⁺=397.29.

Listed in Table 0-16 below are building blocks C that are prepared from D2-3.1 and the corresponding starting material in analogy to the sequence described above for C-16.1.

TABLE C-16

| | | | t_{R} [min] | MS-data |
|---|---|---|---|---|
| | | | LC-MS | m/z |
| No. | SM | Product | Method | [M + H]⁺ |
| C-16.2 | IM-2.5 | N-(N-((Allyloxy)carbonyl)-N-methyl-L-leucyl)-N-(((S)- | 0.56 | 433.14 |
| | | chroman-3-yl)methyl)glycine | I | |
| C-16.3 | IM-2.6 | N-(N-((Allyloxy)carbonyl)-N-methyl-L-leucyl)-N-(((R)- | 0.56 | 433.17 |
| | | chroman-3-yl)methyl)glycine | I | |
| C-16.4 | IM-2.2 | N-(N-((Allyloxy)carbonyl)-N-methyl-L-leucyl)-N-(((R)- | 0.50 | 385.18 |
| | | tetrahydro-2H-pyran-2-yl)methyl)glycine | I | |
| C-16.5 | IM-2.1 | N-(N-((Allyloxy)carbonyl)-N-methyl-L-leucyl)-N-(((S)- | 0.49 | 385.17 |
| | | tetrahydro-2H-pyran-2-yl)methyl)glycine | I | |
| C-16.6 | IM -2.3 | N-(N-((Allyloxy)carbonyl)-N-methyl-L-leucyl)-N-(2-((S)- | 0.52-0.54 | 399.21 |
| | | tetrahydro-2H-pyran-2-yl)ethyl)glycine | I | |
| C-16.7 | IM-2.4 | N-(N-((Allyloxy)carbonyl)-N-methyl-L-leucyl)-N-(2-((R)- | 0.52-0.54 | 399.21 |
| | | tetrahydro-2H-pyran-2-yl)ethyl)glycine | I | |
| C-16.8 | IM-1.16 | N-(N-((Allyloxy)carbonyl)-N-methyl-L-leucyl)-N-((2,3- | 0.95 | 419.37 |
| | | dihydrobenzofuran-2-yl)methyl)glycine | B | |
| C-16.9 | IM-1.14 | N-(N-((Allyloxy)carbonyl)-N-methyl-L-leucyl)-N-((6,6- | 0.98 | 413.43 |
| | | dimethyltetrahydro-2H-pyran-2-yl)methyl)glycine | B | |

### General Method 17 for the Synthesis of Building Blocks C

### N—(N-((Allyloxy)carbonyl)-N-methyl-L-leucyl)-N-methyl-D-phenylalanine (C-17.1)

Step I: PyClop (352 mg, 0.82 mmol) is added to a RT soln. of IM-3.1 (200 mg, 0.682 mmol), D2-3.1 (156 mg, 0.682 mmol), and DIPEA (0.36 mmol, 2.05 mmol) in DCM (3 mL) and the RM is heated to 40° C. overnight. Water (5 mL) is added to the RM and the product is extracted with DCM (2×). The combined org. layers are dried (MgSO₄), filtered, and concentrated. Purification by FC yields methyl N—(N-((allyloxy)carbonyl)-N-methyl-L-leucyl)-N-methyl-D-phenylalaninate. LC-MS B: t_{R}=1.06 min; [M+H]⁺=405.19.

Step 2: LiOH·H₂O (40.1 mg, 0.96 mmol) is added to a RT soln. of methyl N—(N-((allyloxy)carbonyl)-N-methyl-L-leucyl)-N-methyl-D-phenylalaninate (193 mg, 0.478 mmol) in a solvent mix of THF/MeOH/H₂O 2/1/1 (2.5 mL) and the RM is heated to 100° C. for 15 h. THF and MeOH are evaporated and the residue is acidified with 1 M HCl to pH 1, then extracted with EtOAc (3×). The combined org. layers are dried (MgSO₄), filtered, and evaporated to yield C-17.1, which is used as such in the next step. LC-MS 1: t_{R}=0.53-0.56 min; [M+H]⁺=391.23.

Listed in Table C-17 below are building blocks C that are prepared from D2-3.1 and the corresponding starting material in analogy to the sequence described above for C-17.1.

TABLE C-17

| | | | t_{R} [min] | MS-data |
|---|---|---|---|---|
| | | | LC-MS | m/z |
| No. | SM | Product | Method | [M + H]⁺ |
| C-17.2 | IM-3.2 | (R)-2-((S)-2-(((Allyloxy)carbonyl)(methyl)amino)- | 1.03 | 397.25 |
| | | N,4-dimethylpentanamido)-3- | B | |
| | | cyclohexylpropanoic acid | | |
| C-17.3 | IM-3.3 | (R)-2-((S)-2-(((Allyloxy)carbonyl)(methyl)amino)- | 0.96 | 369.13 |
| | | N,4-dimethylpentanamido)-2-cyclopentylacetic | B | |
| | | acid | | |
| C-17.4 | Methyl 1- | (S)-1-(2-(((Allyloxy)carbonyl)(methyl)amino)-N,4- | 0.89 | 355.23 |
| | (methylamino) | dimethylpentanamido)cyclopentane-1-carboxylic | B | |
| | cyclopentane- | acid | | |
| | 1-carboxylate | | | |
| C-17.5 | IM-4.1 | (S)-1-(2-(((Allyloxy)carbonyl)(methyl)amino)-N,4- | 1.00 | 391.17 |
| | | dimethylpentanamido)-3,3-difluorocyclobutane- | B | |
| | | 1-carboxylic acid | | |

### General Method 18 for the Synthesis of Building Blocks C

### 2-((S)-2-((tert-Butoxycarbonyl) (methyl)amino)-N,4-dimethylpentanamido)-3 (4,4-difluoro cyclohexyl)propanoic acid (C-18.1)

Step 1: NaH 60% dispersion in mineral oil (374 mg, 9.76 mmol) is added to a 0° C. suspension of 2-(tert-butoxycarbonylamino)-3-(4,4-difluorocyclohexyl)propanoic acid (1.0 g, 3.25 mmol) in THF (20 mL). The RM is stirred at 0° C. for 10 min, then at RT for another 10 min. The RM is cooled back to 0° C. and Mel (0.614 mL, 9.76 mmol) is added dropwise and the RM is warmed to RT overnight. Water and EtOAc are added, then the two layers are separated. The aq. layer is washed with EtOAc (2×) and the combined org. layers are dried (Na₂SO₄), filtered, and concentrated. Purification by FC (eluting with 0% to 30% EtOAc in hept) yields methyl-2-((tert-butoxycarbonyl)(methyl)amino)-3-(4,4-difluorocyclohexyl)propanoate (434 mg, 40%) as a colourless oil. LC-MS B: t_{R}=1.04 min; [M+H]⁺=336.26.

Step 2: TFA (0.99 mL, 12.9 mmol) is added to a RT soln. of methyl-2-((tert-butoxycarbonyl)(methyl)amino)-3-(4,4-difluorocyclohexyl)propanoate (300 mg, 0.61 mmol) in DCM (10 mL) and the RM is stirred at RT for 2 h.The volatiles are removed in vacuo, and the residue co-evaporated with DCM (3×) to give methyl-3-(4,4-difluorocyclohexyl)-2-(methylamino)propanoate 2,2,2-trifluoroacetate which is used as such in the next step. LC-MS B: t_{R}=0.56 min; [M+H]⁺=236.31.

Steps 3&4: The title compound is prepared from methyl-3-(4,4-difluorocyclohexyl)-2-(methylamino)propanoate 2,2,2-trifluoroacetate and Boc-N-methyl-L-leucine, following the sequence of reactions described for C-2.1, steps 1&2. LC-MS B: t_{R}=1.03 mL; [M+H]⁺=449.27.

Listed in Table C-18 below are building blocks C that are prepared from Boc-N-methyl-L-leucine and the corresponding SM in analogy to the 4-step sequence described above for C-18.1. Alternatively, in step 2, Boc deprotection can be performed in the presence of 4 M HCl in dioxane instead of TFA.

TABLE C-18

| | | | | t_{R} [min] | MS-data |
|---|---|---|---|---|---|
| | | | | LC-MS | m/z |
| No. | SM Step 1 | SM Step 3 | Product | Method | [M + H]⁺ |
| C-18.2 | Boc-O-benzyl- | Boc-N-methyl- | O-Benzyl-N-(N-(tert-butoxycarbonyl)-N- | 1.02 | 451.22. |
| | D-homoserine | L-leucine | methyl-L-leucyl)-N-methyl-D- | B | |
| | | | homoserine | | |
| C-18.3 | Boc-D-Val-OH | Boc-N-methyl- | N-(N-(tert-Butoxycarbonyl)-N-methyl-L- | 0.96 | 359.33 |
| | | L-leucine | leucyl)-N-methyl-D-valine | B | |
| C-18.4 | Boc-D-Leu-OH | Boc-N-methyl- | N-(N-(tert-Butoxycarbonyl)-N-methyl-L- | 1.00 | 373.33 |
| | | L-leucine | leucyl)-N-methyl-D-leucine | B | |
| C-18.5 | (R)-N-Boc-2- | Boc-N-Methyl- | (R)-2-((S)-2-((tert- | 0.92 | 345.31 |
| | aminobutyric | L-leucine | Butoxycarbonyl)(methyl)amino)-N,4- | B | |
| | acid⁺ | | dimethylpentanamido)butanoic acid | | |
| C-18.6 | (R)-Boc-2- | Boc-N-Methyl- | (R)-2-((S)-2-((tert- | 0.93 | 381.26 |
| | amino-4,4- | L-leucine | Butoxycarbonyl)(methyl)amino)-N,4- | B | |
| | difluoro-butyric | | dimethylpentanamido)-4,4- | | |
| | acid⁺ | | difluorobutanoic acid | | |
| C-18.7 | (R)-N-Boc-2- | Boc-N-Methyl- | (R)-2-((S)-2-((tert- | 0.98 | 371.53 |
| | amino-3- | L-leucine | Butoxycarbonyl)(methyl)amino)-N,4- | B | |
| | cyclopropyl- | | dimethylpentanamido)-3- | | |
| | propanoic acid | | cyclopropylpropanoic acid | | |
| C-18.8 | (R)-N-Boc-2- | Boc-N-Methyl- | (R)-2-((S)-2-((tert- | 1.00 | 385.20 |
| | amino-3- | L-leucine | Butoxycarbonyl)(methyl)amino)-N,4- | B | |
| | cyclobutyl- | | dimethylpentanamido)-3- | | |
| | propanoic acid | | cyclobutylpropanoic acid | | |
| C-18.9 | rac-N-Boc-O- | Boc-N-Methyl- | (RS)-N-(N-(tert-Butoxycarbonyl)-N- | 0.92 | 389.37 |
| | ethyl- | L-leucine | methyl-L-leucyl)-O-ethyl-N- | B | |
| | homoserine | | methylhomoserine | | |
| C-18.10 | (R)-N-Boc-2- | Boc-N-Methyl- | (R)-2-((S)-2-((tert- | 1.03 | 387.26 |
| | amino-4,4- | L-leucine | Butoxycarbonyl)(methyl)amino)-N,4- | B | |
| | dimethyl- | | dimethylpentanamido)-4,4- | | |
| | pentanoic acid⁺ | | dimethylpentanoic acid | | |

⁺SM used in step 1 is synthesized from commercially available unprotected amino acids using standard boc protection conditions.

### General Method 19 for the Synthesis of Building Blocks C

### 1-(N-(tert-Butoxycarbonyl)-N-methyl-L-leucyl)-4-phenylpiperidine-2-carboxylic acid (C-19.1)

Step 1: K₂CO₃ (860 mg, 6.22 mmol) is added to a RT soln. of 4-phenyl-piperidine-1,2-dicarboxylic acid 1-tert-butyl ester (1.0 g, 3.11 mmol) and Mel (0.775 mL, 12.4 mmol) in DMF (8 mL) and the RM is stirred at RT for 20 min. Water (10 mL) and DCM (100 mL) are added to the RM, then the layers are separated and the aq. layer is extracted with DCM (2×75 mL). The combined org. layers are washed with brine (50 mL), dried (MgSO₄), filtered, and concentrated to yield 1-(tert-butyl) 2-methyl 4-phenylpiperidine-1,2-dicarboxylate (1.14 g, 105%) as a yellow oil which is used as such in the next step. LC-MS B: t_{R}=1.03 min; [M+H]⁺=320.29.

Step 2: 5 M HCl in ⁱPrOH (3.44 mL, 17.2 mmol) is added to a RT soln. of 1-(tert-butyl) 2-methyl 4-phenylpiperidine-1,2-dicarboxylate (1.10 g, 3.44 mmol) in ⁱPrOH (5 mL) and the RM is stirred at RT for 2 h, then at 50° C. for 30 min. The mix. is concentrated to yield methyl-4-phenylpiperidine-2-carboxylate HCl salt (878 mg, 100%) as a yellow powder which is used as such in the next step. LC-MS B: t_{R}=0.56 min; [M+H]⁺=220.35.

Steps 3&4: The title compound is prepared from methyl-4-phenylpiperidine-2-carboxylate HCl salt and Boc-N-methyl-L-leucine, following the sequence of reactions described for C-2.1, steps 1&2. LC-MS B: t_{R}=1.03 min; [M+H]⁺=449.27.

### General Method 20 for the Synthesis of Building Blocks C

### (R)-2-((S)-2-((tert-Butoxycarbonyl)(methyl)amino)-N,4-dimethylpentanamido)-4-(3-phenyl-1,2,4-oxadiazol-5-yl)butanoic acid (C-20.1)

Step 1: HATU (1.52 g, 4 mmol) is added to a RT soln. of Boc-D-Glu-Ome (1.00 g, 3.64 mmol), N-hydroxyacetamidine (296 mg, 4 mmol), and DIPEA (1.87 mL, 10.9 mmol) in DCM (10 mL) and the RM is stirred at RT for 30 min. The mix. is concentrated to yield the desired intermediate. LC-MS B: t_{R}=0.82 min; [M+H]⁺=380.29. The crude intermediate is dissolved in dioxane (10 mL) and stirred at 80° C. overnight. Water (10 mL) and DCM (100 mL) are added to the RM, then the layers are separated and the aq. layer is extracted with DCM (2×75 mL). The combined org. layers are washed with brine (50 mL), dried (MgSO₄), filtered, and concentrated. Purification by FC (eluting with 10% to 20% EtOAc in hept with R_{f}=0.12 in EtOAc/hept 1:9) yields methyl (R)-2-((tert-butoxycarbonyl)amino)-4-(3-phenyl-1,2,4-oxadiazol-5-yl)butanoate (952 mg, 72%) as a colourless oil. LC-MS B: t_{R}=1.04 min; [M+H]⁺=362.25.

Step 2: Methyl (R)-2-amino-4-(3-phenyl-1,2,4-oxadiazol-5-yl)butanoate HCl salt is prepared from methyl (R)-2-((tert-butoxycarbonyl)amino)-4-(3-phenyl-1,2,4-oxadiazol-5-yl)butanoate following the procedure described for C-19.1, step 2.

Steps 3: Methyl (R)-2-((S)-2-((tert-butoxycarbonyl)(methyl)amino)-4-methylpentanamido)-4-(3-phenyl-1,2,4-oxadiazol-5-yl)butanoate is prepared from methyl (R)-2-amino-4-(3-phenyl-1,2,4-oxadiazol-5-yl)butanoate HCl salt and Boc-N-methyl-L-leucine following the reaction described for C-2.1, step 1. LC-MS B: t_{R}=1.10 min; [M+H]⁺=489.22.

Step 4: NaH (216 mg, 5.65 mmol) is added to a 0° C. soln. of methyl (R)-2-((S)-2-((tert-butoxycarbonyl)(methyl)amino)-4-methylpentanamido)-4-(3-phenyl-1,2,4-oxadiazol-5-yl)butanoate (920 mg, 1.88 mmol) in DMF (9 mL). After 5 min stirring, Mel (0.469 mL, 7.53 mmol) is added to the 0° C. soln., then the ice bath is removed, and the RM stirred at RT for 4 h. Reaction control by LC/MS shows that during the methylation also saponification occurred (t_{R} different than SM). Water (15 mL) and TBME (20 mL) are added to the RM, then the layers are separated and the aq. layer is washed with TBME (1×20 mL). The aq. layer is treated with 2 M HCl (10 mL) and extracted with DCM (2×20 mL). The combined DCM layers are washed with brine (10 mL), dried (MgSO₄), filtered, and concentrated to yield title compound C-20.1 (688 mg, 75%) as a yellow oil, which was used as such in the next step. LC-MS B: t_{R}=1.03 min; [M+H]⁺=489.24.

Listed in Table C-20 are building blocks C, prepared according to the 4-step sequence described above for C-20.1. In cases where saponification does not happen during methylation conditions, an extra step is added to saponify the ester to the carboxylic acid (4 N NaOH in MeOH in analogy to step 3 of C-5.1).

TABLE C-20

| | SM | | | t_{R} [min] | MS-data |
|---|---|---|---|---|---|
| | hydroxyacet- | Conditions | | LC-MS | m/z |
| No. | imidamide* | for Step 4 | Product | Method | [M + H]⁺ |
| C-20.2 | 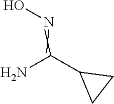 | -20° C. 3 min | (R)-2-((S)-2-((tert- Butoxycarbonyl)(methyl)amino)-N,4- dimethylpentanamido)-4-(3-cyclopropyl- 1,2,4-oxadiazol-5-yl)butanoic acid | 0.97 A | 453.21 |
| C-20.3 | 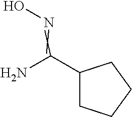 | -20° C. 20 min | (R)-2-((S)-2-((tert- Butoxycarbonyl)(methyl)amino)-N,4- dimethylpentanamido)-4-(3-cyclopentyl- 1,2,4-oxadiazol-5-yl)butanoic acid | 1.04 A | 481.19 |
| C-20.4 | 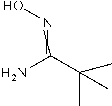 | 0° C. to RT 10 min at RT | (R)-2-((S)-2-((tert- Butoxycarbonyl)(methyl)amino)-N,4- dimethylpentanamido)-4-(3-(tert-butyl)- 1,2,4-oxadiazol-5-yl)butanoic acid | 1.03 A | 469.22 |
| C-20.5 | 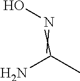 | 0° C. 20 min | (R)-2-((S)-2-((tert- Butoxycarbonyl)(methyl)amino)-N,4- dimethylpentanamido)-4-(3-methyl- 1,2,4-oxadiazol-5-yl)butanoic acid | 0.89 A | 427.25 |

*If not commercially available, hydroxyacetimidamide are synthesized via standard conditions from their corresponding nitrile.

### General Method 21 for the Synthesis of Building Blocks C

### N—(N-(tert-Butoxycarbonyl)-N-methyl-L-leucyl)-N,O-dimethylhomoserine (C-21.1)

Step 1: Mel (0.67 mL, 10.6 mmol is added to a RT soln. of Boc-D-homoserine (2.0 g, 8.85 mmol) and K₂CO₃ (1.85 g, 13.3 mmol) in DMF (30 mL) and the RM is stirred at RT for 16 h. The mix. is poured into ice water and extracted with EtOAc (3×). The combined org. layers are washed with water and brine, dried (MgSO₄), filtered, and concentrated to yield tert-butyl (R)-(2-oxotetrahydrofuran-3-yl)carbamate (2.07 g, 116%) as a light yellow solid. LC-MS B: t_{R}=0.60 min; No ionisation.

Step 2: TFA (3.29 mL, 43 mmol) is added to a RT soln. of tert-butyl (R)-(2-oxotetrahydrofuran-3-yl)carbamate (2.0 g, 8.6 mmol) in DCM (20 mL) and the RM is stirred at RT for 6 h. The volatiles are removed in vacuo, co-evaporated with DCM (3×) to give the crude (R)-3-aminodihydrofuran-2 (3H)-one 2,2,2-trifluoroacetate (3.0 g, 160%) which is used as such in the next step. By LC-MS no product formation could be detected, only the disappearance of SM is followed.

Step 3: HATU (5.83 g, 15.3 mmol) is added to a RT soln. of (R)-3-aminodihydrofuran-2 (3H)-one 2,2,2-trifluoroacetate (3.00 g), Boc-N-methyl-L-leucine (3.70 g, 14.6 mmol) and DIPEA (7.16 mL, 41.8 mmol) in DMF (20 mL) and the RM is stirred for 1 h. The mix. is partitioned between water and EtOAc and the layers are separated, and the aq. layer is re-extracted with EtOAc (2×). The combined org. extracts are washed with water and brine, dried (Na₂SO₄), filtered, and evaporated. Purification by FC (eluting with 50% EtOAc in hept) yields tert-butyl methyl((S)-4-methyl-1-oxo-1-(((R)-2-oxotetrahydrofuran-3-yl)amino)pentan-2-yl)carbamate (2.21 g, 75%) as a colourless oil. LC-MS B: t_{R}=0.87 min; [M+H]⁺=329.28.

Step 4: An aq. 8 M NaOH soln. (1.7 mL, 13.5 mmol) is added to a RT soln. of tert-butyl methyl((S)-4-methyl-1-oxo-1-(((R)-2-oxotetrahydrofuran-3-yl)amino)pentan-2-yl)carbamate (2.21 g, 6.73 mmol) in dioxane (20 mL) and the RM is stirred at 50° C. for 1 h. The RM is concentrated to dryness, then the crude is taken up in DCM and acidified with a 2 N aq. HCl soln. (pH 3). The layers are separated and the aq. layer is extracted with DCM (3×). The combined org. layers are washed with brine, dried (MgSO₄), filtered, and concentrated to yield N-(tert-butoxycarbonyl)-N-methyl-L-leucylhomoserine (2.40 g, 100%) as a colourless oil, which is used as such in the next step. Epimerization occurred during lactone opening. LC-MS B: t_{R}=0.75 min; [M+H]⁺=347.30.

Step 5: NaH 60% dispersion in mineral oil (796 mg, 20.8 mmol) is added portionwise to a RT soln. of N-(tert-butoxycarbonyl)-N-methyl-L-leucylhomoserine (2.40 mg, 6.93 mmol) in DMF (50 mL), then Mel (1.74 mL, 27.7 mmol) is added and stirring at RT continued for 1 h. The mix. is partitioned between water and EtOAc. The layers are separated and the aq. layer re-extracted with EtOAc (2×). The combined org. extracts are washed with water and brine, dried (Na₂SO₄), filtered, and evaporated. Purification by FC (eluting with 100% EtOAc) yields methyl N—(N-(tert-butoxycarbonyl)-N-methyl-L-leucyl)-N,O-dimethylhomoserinate (692 mg, 26%) as a yellowish oil. LC-MS B: t_{R}=0.99 min; [M+H]⁺=389.26.

Step 6: An aq. 8 M NaOH soln. (1.7 mL, 3.56 mmol) is added to a RT soln. of methyl N—(N-(tert-butoxycarbonyl)-N-methyl-L-leucyl)-N,O-dimethylhomoserinate (692 mg, 1.78 mmol, 1 eq) in dioxane (20 mL) and the RM is stirred at 50° C. for 1 h. The RM is concentrated to dryness, then the crude is taken up in DCM and acidified with a 2 N aq. HCl soln. (pH 3). The layers are separated, and the aq. layer is extracted with DCM (3×). The combined org. layers are washed with brine, dried (MgSO₄), filtered, and concentrated to yield the title compound C-21.1 (750 mg, 112%) as a colourless oil which is used as such in the next step. LC-MS B: t_{R}=0.88 min; [M+H]⁺=375.32.

### General Method 22 for the Synthesis of Building Blocks C

### (R)-2-((S)-2-((tert-Butoxycarbonyl)(methyl)amino)-N,4-dimethylpentanamido)-3-(3-(5-fluoropyridin-2-yl)-1,2,4-oxadiazol-5-yl)propanoic acid (C-22.1)

Step 1: Benzyl bromide (4.71 mL, 38.8 mmol) is added to a RT solution of Boc-D-Asp-OMe (10.00 g, 38.8 mmol) and DIPEA (26.6 mL, 155 mmol) in DMF (71 mL) and the RM is heated to 50° C. for 2 h. The solution is allowed to reach RT, then water and Et₂O are added and the layers separated. The inorg. layer is extracted with Et₂O (1×). The combined org. layers are washed with brine, dried (MgSO₄), filtered, and concentrated. Purification by FC (eluting with 0% to 30% EtOAc in hept with R_{f}=0.24 in EtOAc/hept 1:4) yields 4-benzyl 1-methyl (tert-butoxycarbonyl)-D-aspartate (12.7 g, 97%) as a colorless oil. LC-MS B: t_{R}=0.97 min; [M+H]⁺=337.96.

Step 2: 4 M HCl in dioxane (57.9 mL, 240 mmol) is added to a RT solution of 4-benzyl 1-methyl (tert-butoxycarbonyl)-D-aspartate (8.19 g, 24 mmol) in dioxane (42.3 mL) and the resulting RM is heated to 50° C. for 30 min. The mixture is allowed to reach RT, then concentrated to yield 4-benzyl 1-methyl D-aspartate HCl-salt (6.90 g, 92%) of a yellowish solid which is used as such in the next step. LC-MS B: t_{R}=0.53 min; [M+H]⁺=238.30.

Step 3: HATU (10.38 g, 26.5 mmol) is added to a RT solution of 4-benzyl 1-methyl D-aspartate HCl-salt (6.84 g, 22.1 mmol), boc-N-methyl-L-leucine (5.58 g, 22.1 mmol), and DIPEA (19.9 mL, 110 mmol) in MeCN (83 mL). The resulting mixture is stirred at RT for 10 min. Water (135 mL) and DCM (315 mL) is added to the RM, then the layers are separated and the inorg. layer is extracted with DCM. The combined org. layers are washed with brine (50 mL), dried over a phase separator and concentrated. Purification by FC (eluting with 0% to 40% EtOAc) yields 4-benzyl 1-methyl N-(tert-butoxycarbonyl)-N-methyl-L-leucyl-D-aspartate (9.70 g, 94%) as a yellowish oil. LC-MS B: t_{R}=1.10 min; [M+H]⁺=465.03.

Step 4: NaH (1.02 g, 26.5 mmol) is added to a −20° C. solution of 4-benzyl 1-methyl N-(tert-butoxycarbonyl)-N-methyl-L-leucyl-D-aspartate (4.53 g, 8.83 mmol) and Mel (2.22 mL, 35.3 mmol) in DMF (73 mL). The resulting solution is stirred at −20° C. for 15 min, then quenched with 1 M aq. HCl soln. (224 mL) and diluted with isopropyl acetate. The layers are separated and the inorg. layer is extracted with isopropyl acetate (1×). The combined org. layers are dried (MgSO4), filtered, and concentrated. Purification by FC (eluting with 10% to 40% EtOAc in hept with R_{f}=0.31 in EtOAc/hept 1:1) yields 4-benzyl 1-methyl N—(N-(tert-butoxycarbonyl)-N-methyl-L-leucyl)-N-methyl-D-aspartate (3.9 g, 92%) as a yellowish oil. LC-MS B: t_{R}=1.11 min; [M+H]⁺=479.16.

Step 5: Pd/C (10%, 387 mg, 0.364 mmol) is added to a RT soln. of 4-benzyl 1-methyl N—(N-(tert-butoxycarbonyl)-N-methyl-L-leucyl)-N-methyl-D-aspartate (3.89 g, 7.28 mmol) in MeOH (34 mL) and the RM is stirred at RT for 1 h under a H₂ atm. The RM is filtered and concentrated to yield (R)-3-((S)-2-((tert-butoxycarbonyl)(methyl)amino)-N,4-dimethylpentanamido)-4-methoxy-4-oxobutanoic acid (3.2 g, 114%) as a colorless oil, which is used as such in the next step. LC-MS B: t_{R}=0.89 min; [M+H]⁺=389.33.

Step 6: PyBOP (1.73 g, 3.25 mmol) is added to a RT soln. of (R)-3-((S)-2-((tert-butoxycarbonyl)(methyl)amino)-N,4-dimethylpentanamido)-4-methoxy-4-oxobutanoic acid (1.00 g, 2.5 mmol), 5-fluoro-N′-hydroxypicolinimidamide (631 mg, 3.75 mmol), and DIPEA (1.28 mL, 7.49 mmol) in DCM (2.8 mL). The resulting RM is stirred at RT for 10 min. The solvent is removed in vacuo and the residue re-dissolved in dioxane (2.8 mL). The resulting mix. is heated to 100° C. for 3.5 h, then to 90° C. overnight. Next morning, the heating was increased to 100° C. for another 8 h. The RM is allowed to reach RT before concentrated in vacuo, then water and DCM is added. The layers are separated and the inorg. layer is extracted with DCM (1×). The combined org. layers are dried over a phase separator and concentrated. Purification by FC (eluting with 10% to 50% EtOAc in hept with R_{f}=0.36 in EtOAc/hept 1:1) yields methyl (R)-2-((S)-2-((tert-butoxycarbonyl)(methyl)amino)-N,4-dimethylpentanamido)-3-(3-(5-fluoropyridin-2-yl)-1,2,4-oxadiazol-5-yl)propanoate (931 g, 73%) as a yellowish solid. LC-MS B: t_{R}=1.04 min; [M+H]⁺=508.18.

Step 7: 4 M NaOH soln. (3.70 mL, 14.8 mmol) is added to a RT soln. of methyl (R)-2-((S)-2-((tert-butoxycarbonyl)(methyl)amino)-N,4-dimethylpentanamido)-3-(3-(5-fluoropyridin-2-yl)-1,2,4-oxadiazol-5-yl)propanoate (1.50 g, 2.96 mmol) in MeOH (15 mL), and the RM is stirred at RT for 15 min. The RM is diluted with DCM (50 mL) and acidified with 2 M HCl soln. (20 mL). The layers are separated and the inorg. layer is extracted with DCM. The combined org. layers are washed with brine (30 mL), dried (MgSO₄), filtered, and concentrated to yield the title compound C-22.1 (1.40 g, 96%) as an off-white foam which is used as such in the next step. LC-MS B: t_{R}=0.95 min; [M+H]⁺=494.18.

### (R)-2-((S)-2-((tert-Butoxycarbonyl)(methyl)amino)-N,4-dimethylpentanamido)-3-(3-(trifluoromethyl)-1,2,4-oxadiazol-5-yl)propanoic acid (C-22.2)

The title compound is prepared following the 7-step sequence described for C-22.1, using in step 6, commercially available 2,2,2-trifluoro-N′-hydroxyacetimidamide instead of 5-fluoro-N′-hydroxypicolinimidamide. LC-MS B: t_{R}=1.02 min; [M+H]⁺=467.11.

Listed in Table 0-22 below are building blocks C, prepared according to the 7-step sequence described above for C-22.1. In step 6, (R)-3-((S)-2-((tert-butoxycarbonyl)(methyl)amino)-N,4-dimethylpentanamido)-4-methoxy-4-oxobutanoic acid and their corresponding hydroxyacetimidamide are used. For C-22.5, synthesis is performed in analogy to C-22.1, using in step 1 boc-D-Glu-OMe instead of boc-D-Asp-OMe and in step 6 the corresponding hydroxyacetimidamide as outlined in Table 0-22.

TABLE C-22

| | | | tR [min] | MS-data |
|---|---|---|---|---|
| | SM | | LC-MS | m/z |
| No. | hydroxyacetimidamide* | Product | Method | [M + H]⁺ |
| C-22.3 | 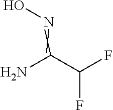 | (R)-2-((S)-2-((tert- Butoxycarbonyl)(methyl)amino)-N,4- dimethylpentanamido)-3-(3- (difluoromethyl)-1,2,4-oxadiazol-5- yl)propanoic acid | 0.95 A | 449.10 |
| C-22.4 | 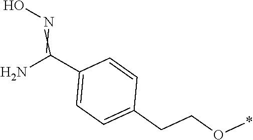 [SMILES: *OCCc1ccc(C(N)=NO)cc1] | (R)-2-((S)-2-((tert- Butoxycarbonyl)(methyl)amino)-N,4- dimethylpentanamido)-3-(3-(4-(2- methoxyethyl)phenyl)-1,2,4- oxadiazol-5-yl)propanoic acid | 1.06 A | 533.17 |
| C-22.5 | 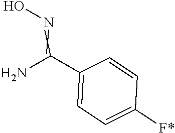 | (R)-2-((S)-2-((tert- Butoxycarbonyl)(methyl)amino)-N,4- dimethylpentanamido)-4-(3-(5- fluoropyridin-2-yl)-1,2,4-oxadiazol-5- yl)butanoic acid | 0.97 A | 508.21 |

*If not commercially available, hydroxyacetimidamide are synthesized via standard conditions from their corresponding nitrile.

### General Method C-23 for the Synthesis of Building Blocks C

### 2-((S)-2-((tert-Butoxycarbonyl)(methyl)amino)-N,4-dimethylpentanamido)-3-(3,3-difluoropyrrolidin-1-yl)propanoic acid (C-23.1)

Step 1: NaH (1.02 g, 25.4 mmol) is added in four portions over 2 h, to a 000 solution of benzyl (tert-butoxycarbonyl)-L-serinate (1.50 g, 5.08 mmol) and Mel (1.59 mL, 25.4 mmol) in THF (30 mL). The ice bath is removed and the RM stirred at RT for 2 h. The mixture is cooled to 0° C., then water is added carefully (strong gas evolution). Mixture is washed with Et₂O and organic layer is discarded. The water layer is acidified with 10% citric acid solution, then extracted with EtOAc (2×). The combined org. layers are washed with brine, dried (Na₂SO₄), filtered, and concentrated to yield 2-((tert-butoxycarbonyl)(methyl)amino)acrylic acid (1.178 g). Acetone (30 mL) is added to this crude material, followed by K₂CO₃ (1.81 g, 13.08 mmol), and benzyl bromide (1.04 mL, 8.72 mmol). The mixture is stirred at 50° C. for 50 min, then the suspension is diluted with water, partially concentrated to remove the acetone, then extracted with EtOAc (3×). The combined org. layers are washed with brine, dried (Na₂SO₄), filtered, and concentrated. Purification by FC (eluting with 5% to 100% EtOAc in hept) yields benzyl 2-((tert-butoxycarbonyl)(methyl)amino)acrylate (760 mg, 59%) as a yellow oil. LC-MS J: t_{R}=2.13 min; [M+H]⁺=no ionization.

Step 2: K₂CO₃ (378 mg, 3.78 mmol) is added to a solution of 3,3-difluoropyrrolidine HCl (493 mg, 3.43 mmol) in 2-propanol (0.69 mL) and water (61.8 μl, 3.43 mmol), then benzyl 2-((tert-butoxycarbonyl)(methyl)amino)acrylate (200 mg, 0.686 mmol) is added the resulting RM is heated to 60° C. for 18 h. The solution is concentrated, then sat. NaHCO₃ soln. and EtOAc are added. The org. layer is collected and the inorg. layer is extracted with EtOAc (2×). The combined org. layers are washed with brine, dried (Na₂SO₄), filtered, and concentrated. Purification by prep. HPLC (basic) yields benzyl 2-((tert-butoxycarbonyl)(methyl)amino)-3-(3,3-difluoropyrrolidin-1-yl)propanoate (321 mg, 117%) as a yellowish oil. LC-MS J: t_{R}=2.11 min; [M+H]⁺=399.20

Step 3: TFA (2.0 mL, 26.0 mmol) is added to a RT soln. of benzyl 2-((tert-butoxycarbonyl)(methyl)amino)-3-(3,3-difluoropyrrolidin-1-yl)propanoate (0.140 g, 0.320 mmol) in DCM (2.0 mL) and the RM is stirred at RT for 2 h. The volatiles are removed in vacuo, and the residue co-evaporated with DCM (2×) to give benzyl 3-(3,3-difluoropyrrolidin-1-yl)-2-(methylamino)propanoate TFA-salt as an oil which is used as such in the next step.

Steps 4 and 5: The title compound is prepared from Boc-N-methyl-L-leucine and benzyl 3-(3,3-difluoropyrrolidin-1-yl)-2-(methylamino)propanoate TFA-salt following the sequence of reactions described for C-1.1, step 3 and 4. LC-MS F: t_{R}=1.93 min; [M+H]⁺=436.20.

### 2-((S)-2-((tert-Butoxycarbonyl)(methyl)amino)-N,4-dimethylpentanamido)-3-(pyrrolidin-1-yl)propanoic acid (C-23.2)

The title compound is prepared following the 5-step sequence described for C-23.1, using in step 2 following conditions. Pyrrolidine (0.213 mL, 2.57 mmol) is added to a RT solution of benzyl 2-((tert-butoxycarbonyl)(methyl)amino)acrylate (500 mg, 1.716 mmol) in EtOH (10 mL) and the RM is heated to 50° C. for 1.5 h. The RM is concentrated and directly purified by prep HPLC to yield desired intermediate (53 mg, 76%) as a clear oil. Following step 3 to 5 (as described for C-23.1) yields desired title compound C-23.2. LC-MS J: t_{R}=1.69 min; [M+H]⁺=400.30

### General Method 24 for the Synthesis of Building Blocks C

### (R)-2-((S)-2-((tert-Butoxycarbonyl)(methyl)amino)-N,4-dimethylpentanamido)-3-(3-phenyl-1,2,4-oxadiazol-5-yl)propanoic acid (C-24.1)

The title compound is prepared following the 4-step sequence described for C-20.1, using Boc-D-Asp-OMe instead of Boc-D-Glu-OMe in step 1. In most cases, a saponification step (4 N NaOH in MeOH) is added. LC-MS A: t_{R}=1.04 min; [M+H]⁺=475.23

TABLE C-24

| | | | t_{R} [min] | MS-data |
|---|---|---|---|---|
| | SM | | LC-MS | m/z |
| No. | hydroxyacetimidamide* | Product | Method | [M + H]⁺ |
| C-24.2 | 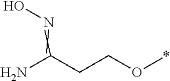 [SMILES: *OCCC(N)=NO] | (R)-2-((S)-2-((tert-Butoxycarbonyl)(methyl)amino)- N,4-dimethylpentanamido)-3-(3-(2-methoxyethyl)- 1,2,4-oxadiazol-5-yl)propanoic acid | 1.00 A | 471.42 |
| C-24.3 | 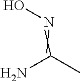 | (R)-2-((S)-2-((tert-Butoxycarbonyl)(methyl)amino)- N,4-dimethylpentanamido)-3-(3-methyl-1,2,4- oxadiazol-5-yl)propanoic acid | 0.88 A | 413.25 |

*If not commercially available, hydroxyacetimidamide are synthesized via standard conditions from their corresponding nitrile.

### General Method 25 for the Synthesis of Building Blocks C

### (R)-2-((S)-2-((tert-Butoxycarbonyl)(methyl)amino)-N,4-dimethylpentanamido)-3-(3-methoxyisoxazol-5-yl)propanoic acid (C-25.1)

Step 1: Ethyl chloroformate (3.54 mL, 36.4 mmol) is added dropwise to a 0° C. solution of 3-methoxy-1,2-oxazole-5-carboxylic acid (4.98 g, 33.1 mmol) and TEA (5.29 mL, 38.0 mmol) in THF (132 mL). The RM is stirred at 0° C. for 1.5 h, then a solution of NaBH₄ (3.79 g, 99.2 mmol) in water (53 mL) is added dropwise at 0° C. and the resulting RM stirred at 0° C. for 10 min. The RM is allowed to warm up to RT, then DCM is added, and the two layers are separated. The inorg. layer is extracted with DCM (2×), and the combined org. layers are washed with brine, dried (over phase separator), and concentrated. Purification by FC (eluting with 10% to 50% EtOAc in hept., with R_{f}=0.38 in hept./EtOAc 1:1) gives (3-methoxyisoxazol-5-yl)methanol (1.39 g, 33%) as a colorless oil. LC-MS A: t_{R}=0.39 min; [M+H]⁺=130.10.

Step 2: PBr₃ (1.03 mL, 10.8 mmol) is added dropwise to a 0° C. solution of (3-methoxyisoxazol-5-yl)methanol (1.39 g, 10.8 mmol) in Et₂O (115 mL). The ice bath is removed and the resulting RM is stirred at RT for 48 h. The RM is diluted with EtOAc and sat. aq. Na₂CO₃ soln. The layers are separated and the inorg. layer is extracted with EtOAc (1×). The combined org. layers are dried (MgSO₄), filtered, and concentrated to yield 5-(bromomethyl)-3-methoxyisoxazole (1.65 g, 80%) as a slightly yellow oil which is used as such in the next step. LC-MS A: t_{R}=0.71 min; [M+H]⁺=no ionization.

Step 3: n-BuLi (1.6 M in hexanes, 6.03 mL, 9.53 mmol) was added dropwise to a −75° C. solution of (S)-2,5-dihydro-3,6-dimethoxy-2-isopropylpyrazine (1.5 mL, 8.1 mmol) in THF (74 mL). The resulting solution is stirred for 30 min, then a solution of 5-(bromomethyl)-3-methoxyisoxazole (1.64 g, 8.1 mmol) in THF (60 mL) is added and the RM is stirred at −75° C. for 1.5 h. 1 M aq. NH₄Cl soln (86 mL) and Et₂O is added to the RM, the two layers are separated and the inorg. layer is dried (MgSO₄), filtered, and concentrated. Purification by FC (eluting with 0% to 20% of EtOAc in hept., R_{f}=0.32 in hept/EtOAc 4:1) yields 5-(((2R,5S)-5-isopropyl-3,6-dimethoxy-2,5-dihydropyrazin-2-yl)methyl)-3-methoxyisoxazole (1.30 g, 55%) as a slightly yellow oil. LC-MS A: t_{R}=0.91 min; [M+H]⁺=296.24.

Step 4: 1 M aq. HCl soln (8.1 mL, 8.21 mmol) is added to a RT solution of 5-(((2R,5S)-5-isopropyl-3,6-dimethoxy-2,5-dihydropyrazin-2-yl)methyl)-3-methoxyisoxazole (1.31 g, 4.1 mmol) in MeCN (41.4 mL). The RM is stirred at RT for 30 min, then the solvent is removed, and the residue treated with 1 M aq. NH₃ soln. until pH=9. DCM is added to the RM and the two layers are separated. The inorg. layer is extracted with DCM (1×) and the combined org. layers are dried (over phase separator) and concentrated. Purification by FC (eluting with 0 to 2% MeOH in DCM (containing 0.5% NH₄OH), with R_{f}=0.20 in DCM/MeOH/NH₄OH 100:2:0.5) yields methyl (R)-2-amino-3-(3-methoxyisoxazol-5-yl)propanoate (756 mg, 92%) as a yellow oil. LC-MS A: t_{R}=0.35 min; [M+H]⁺=201.34.

Step 5: HATU (1.648 g, 4.20 mmol) is added to a RT solution of methyl (R)-2-amino-3-(3-methoxyisoxazol-5-yl)propanoate (750 mg, 3.50 mmol), boc-N-methyl-L-leucine (886 mg, 3.5 mmol), and DIPEA (1.8 mL, 10.5 mmol) in MeCN (12.8 mL). The RM is stirred at RT for 10 min, then water and DCM are added, and the layers are separated. The inorg. layers are extracted with DCM (1×) and the combined org. layers are dried (over phase separator) and concentrated. Purification by FC (eluting with 10% to 40% EtOAc in hept., with R_{f}=0.23 in hept/EtOAc 7:3) yields methyl (R)-2-((S)-2-((tert-butoxycarbonyl)(methyl)amino)-4-methylpentanamido)-3-(3-methoxyisoxazol-5-yl)propanoate (1.40 g, 94%) as a colorless oil. LC-MS A: t_{R}=0.99 min; [M+H]⁺=428.19.

Step 6: NaH (376 mg, 9.82 mmol) is added to a −20° C. solution of methyl (R)-2-((S)-2-((tert-butoxycarbonyl)(methyl)amino)-4-methylpentanamido)-3-(3-methoxyisoxazol-5-yl)propanoate (1.40 g, 3.27 mmol) and Mel (0.82 mL, 13.1 mmol) in DMF (28 mL). The RM is stirred at −20° C. for 11 min, then the RM is quenched with 1 M aq. HCl soln (84 mL) followed by the addition of Et₂O. The layers are separated and the inorg. layer is extracted with Et₂O (1×). The combined org. layers are dried (MgSO₄), filtered, and concentrated. Purification by FC (eluting with 5% to 40% of EtOAc in hept., with R_{f}=0.26 in hept/EtOAc 7:3) yields methyl (R)-2-((S)-2-((tert-butoxycarbonyl)(methyl)amino)-N,4-dimethylpentanamido)-3-(3-methoxyisoxazol-5-yl)propanoate (1.30 g, 90%) as a yellowish oil. LC-MS A: t_{R}=1.03 min; [M+H]⁺=442.20.

Step 7: 4 M aq. NaOH soln (28.8 mL, 114 mmol) is added to a RT solution of methyl (R)-2-((S)-2-((tert-butoxycarbonyl)(methyl)amino)-N,4-dimethylpentanamido)-3-(3-methoxyisoxazol-5-yl)propanoate (1.303 g, 2.86 mmol) in MeOH (41.2 mL). The RM is heated to 50° C. and stirred at this temperature for 10 min, then the RM is allowed to reach RT. 2 M aq. HCl soln (165 mL) and DCM (105 mL) are added, the layers separated, and the inorg. layer extracted with DCM (1×). The combined org. layers are dried (over phase separator) and concentrated to yield the title compound (1.20 g, 99%) as a colorless oil. LC-MS A: t_{R}=0.92 min; [M+H]⁺=428.17.

### (R)-2-((S)-2-((tert-Butoxycarbonyl)(methyl)amino)-N,4-dimethylpentanamido)-3-(3-(tert-butyl)isoxazol-5-yl)propanoic acid (C-25.2)

The title compound is prepared following the 7-step sequence described for C-25.1, using in step 1 3-tert-butyl-1,2-oxazole-5-carboxylic acid instead of 3-methoxy-1,2-oxazole-5-carboxylic acid. LC-MS A: t_{R}=1.14 min; [M+H]⁺=468.46.

### (R)-2-((S)-2-((tert-Butoxycarbonyl)(methyl)amino)-N,4-dimethylpentanamido)-3-(5-(tert-butyl)-1,2,4-oxadiazol-3-yl)propanoic acid (C-25.3)

The title compound is prepared following step 3 to step 7, described for C-25.1, using in step 3, 5-(tert-butyl)-3-(chloromethyl)-1,2,4-oxadiazole instead of 5-(bromomethyl)-3-methoxyisoxazole. LC-MS A: t_{R}=1.00 min; [M+H]⁺=455.09.

### General Method 26 for the Synthesis of Building Blocks C

### (R)-2-((S)-2-((tert-Butoxycarbonyl)(methyl)amino)-N,4-dimethylpentanamido)-3-(4-phenyl-1H-1,2,3-triazol-1-yl)propanoic acid (26.1)

Step 1: Copper(II) acetate (66.5 mg, 0.348 mmol) is added to a RT solution of 3-azido-N-Boc-D-alanine methyl ester (850 mg, 3.48 mmol), phenylacetylene (0.39 mL, 3.48 mmol), and (+)-sodium L-ascorbate (141 mg, 0.696 mmol) in a 1:1 mixture of tBuOH/H₂O (11.9 mL), then the RM is stirred at RT for 20 min. The RM is diluted with DCM and brine, the layers are separated and the org. layer is dried (with phase separator) and concentrated to yield methyl (R)-2-((tert-butoxycarbonyl)amino)-3-(4-phenyl-1H-1,2,3-triazol-1-yl)propanoate (1.07 g, 89%) a slightly yellowish solid. LC-MS A: t_{R}=0.89 min; [M+H]⁺=347.19.

Step 2: 4 M HCl in dioxane (7.6 mL, 30.7 mmol) is added to a RT solution of methyl (R)-2-((tert-butoxycarbonyl)amino)-3-(4-phenyl-1H-1,2,3-triazol-1-yl)propanoate (1.06 g, 3.07 mmol) in dioxane (5.4 mL). The RM is heated to 50° C. for 30 min. The RM is allowed to reach RT, then the RM is concentrated to yield methyl (R)-2-amino-3-(4-phenyl-1H-1,2,3-triazol-1-yl)propanoate dihydrochloride (980 mg, 100%) of a yellowish solid. LC-MS A: t_{R}=0.49 min; [M+H]⁺=247.28.

Step 3: HATU (1.437 g, 3.67 mmol) is added to a RT solution of methyl (R)-2-amino-3-(4-phenyl-1H-1,2,3-triazol-1-yl)propanoate dihydrochloride (975 mg, 3.05 mmol), boc-N-methyl-L-leucine (773 mg, 3.05 mmol) and DIPEA (2.61 mL, 15.3 mmol) in MeCN (10.9 mL). The RM is stirred at RT for 10 min, then water and DCM are added. The layers are separated, the inorg. layer extracted with DCM (1×), and the combined org. layers are dried (over phase separator) and concentrated. Purification by FC (eluting with 20% to 60% EtOAc in hept. with R_{f}=0.28 in EtOAc/hept 1:1) yields methyl (R)-2-((S)-2-((tert-butoxycarbonyl)(methyl)amino)-4-methylpentanamido)-3-(4-phenyl-1H-1,2,3-triazol-1-yl)propanoate (1.90 g, 130%) as an orange oil. LC-MS A: t_{R}=1.04 min; [M+H]⁺=474.17.

Step 4: NaH (381 mg, 9.95 mmol) is added to a −20° C. solution of methyl (R)-2-((S)-2-(tert(-butoxycarbonyl)(methyl)amino)-4-methylpentanamido)-3-(4-phenyl-1H-1,2,3-triazol-1-yl)propanoate (1.772 g, 3.32 mmol) and Mel (0.834 mL, 13.3 mmol) in DMF (28.2 mL). The RM is stirred at −20° C. for 17 min, then quenched with 1 M aq. HCl soln (84.6 mL) and Et₂O. The layers are separated and the inorg. layers are extracted with Et₂O (1×). The combined org. layers are dried (MgSO₄), filtered, and concentrated. Purification by FC (eluting with 0% to 60% EtOAc in hept. with R_{f}=0.52 in hept/EtOAc 4:6) yields methyl (R)-2-((S)-2-((tert-butoxycarbonyl)(methyl)amino)-N,4-dimethylpentanamido)-3-(4-phenyl-1H-1,2,3-triazol-1-yl)propanoate (612 mg, 39%) as a yellow oil. LC-MS A: t_{R}=1.07 min; [M+H]⁺=488.19.

Step 5: 4 M aq. NaOH soln (10.3 mL, 40.7 mmol) is added to a RT solution of methyl (R)-2-((S)-2-((tert-butoxycarbonyl)(methyl)amino)-N,4-dimethylpentanamido)-3-(4-phenyl-1H-1,2,3-triazol-1-yl)propanoate (607 mg, 1.02 mmol) in MeOH (14.4 mL). The RM is heated to 50° C. for 12 min, then allowed to reach RT before 2 M aq. HCl soln (59 mL) and DCM (35 mL) are added. The layers are separated, and the inorg. layer is extracted with DCM (1×). The combined org. layers are dried (over phase separator) and concentrated. Purification by prep HPLC (acidic conditions) yields title compound (337 mg, 70%) as a white foam. LC-MS A: t_{R}=0.97 min; [M+H]⁺=474.18.

### (R)-2-((S)-2-((tert-Butoxycarbonyl)(methyl)amino)-N,4-dimethylpentanamido)-3-(4-(5-fluoropyridin-2-yl)-1H-1,2,3-triazol-1-yl)propanoic acid (C-26.2)

The title compound is prepared following the 5-step sequence described for C-26.1, using in step 1 2-ethynyl-5-fluoropyridine instead of phenylacetylene. LC-MS A: t_{R}=0.94 min; [M+H]⁺=493.36.

### General Method 27 for the Synthesis of Building Blocks C

### N—(N-(tert-Butoxycarbonyl)-N-methyl-L-leucyl)-O-cyclohexyl-N-methyl-D-homoserine (C-27.1)

Step 1: To a suspension of sodium hydride (suspension in oil, 60%) (1017 mg, 26.5 mmol) in DMF (10 mL) is added dropwise a solution of Boc-D-homoserine (3000 mg, 13.3 mmol) in DMF (10 mL) at 0° C. and the solution is stirred for 20 min. To this solution is added tetrabutylammonium iodide (49.5 mg, 0.133 mmol) and the resulting mixture is stirred for 30 min, and 3-Bromocyclohexene (2 mL, 15.6 mmol) is added. After stirring for 2 h at RT, sodium iodide (2010 mg, 13.3 mmol) is added and the RM is stirred at rt overnight. Water and EtOAc are added and the 2 phases are separated. The inorg. layer is extracted further with EtOAc (2×). The combined org. layers are dried over Na₂SO₄, filtered and concentrated to yield N-(tert-butoxycarbonyl)-O-(cyclohex-2-en-1-yl)-D-homoserine (3.06 g) as a brown oil which is used as such in the next step. LC-MS B. t_{R}=0.86 min; [M+H]⁺=300.33.

Step 2: Under argon, N-(tert-butoxycarbonyl)-O-(cyclohex-2-en-1-yl)-D-homoserine (3060 mg, 10.2 mmol) is solubilized in a mixture of DCM (10 mL) and MeOH (10 mL). This solution is cooled down to 0° C. and trimethylsilyldiazomethane (ca. 10% in Hexane, ca. 0.6 mol/L) (21 mL, 12.3 mmol) is added slowly. The solution is stirred for 1h at RT. Solvents are removed to dryness. The crude material is absorbed over Isolute and purified via FC, using a gradient of Heptane/EtOAc from 100:0 to 60:40 to give 0.9 g of the methyl N-(tert-butoxycarbonyl)-O-(cyclohex-2-en-1-yl)-D-homoserinate as a light yellow oil. LC-MS B: t_{R}=0.98 min; [M+H]⁺=314.34.

Step 3: Under N₂, methyl N-(tert-butoxycarbonyl)-O-(cyclohex-2-en-1-yl)-D-homoserinate (900 mg, 2.87 mmol) is dissolved in MeOH (20 mL); the vessel is purged with N₂/vacuum (3×) before 10% Pd/C (90 mg) is added. After inertising another three times a H₂ balloon is connected and the RM is stirred for 1 h under H₂ atmosphere. The heterogeneous reaction mixture is filtered over a glass fiber filter (washing with methanol/THF). The filtrate is then concentrated to dryness under reduced pressure to yield 0.82 g of methyl N-(tert-butoxycarbonyl)-O-cyclohexyl-D-homoserinate as a colorless oil which is used as such in the next step. LC-MS B: t_{R}=1.01 min; [M+H]⁺=316.37.

Step 4: Sodium hydride (60% dispersion in mineral oil) (149 mg, 3.9 mmol) is added portionwise to a RT solution of methyl N-(tert-butoxycarbonyl)-O-cyclohexyl-D-homoserinate (820 mg, 2.6 mmol) and iodomethane (0.245 mL, 3.9 mmol) in DMF (8 mL) under argon and the resulting mixture is stirred for 1h. The RM is partitioned between water and EtOAc. The layers are separated and the inorg. layer is extracted further with EtOAc (2×). The combined org. extracts are washed with water and brine, dried over Na₂SO₄, filtered and evaporated in vacuo to give 0.84 g of methyl N-(tert-butoxycarbonyl)-O-cyclohexyl-N-methyl-D-homoserinate as a yellowish oil. No purification at this stage. LC-MS B: t_{R}=1.06 min; [M+H]⁺=330.37.

Step 5 to 7: The title compound is prepared as a colorless oil from Boc-N-methyl-L-leucine and methyl N-(tert-butoxycarbonyl)-O-cyclohexyl-N-methyl-D-homoserinate following the sequence of reactions described for C-18.1, step 2 to 4. LC-MS B: t_{R}=1.06 min; [M+H]⁺=443.44.

### N—(N-(tert-Butoxycarbonyl)-N-methyl-L-leucyl)-N-methyl-O-phenyl-D-homoserine (C-27.2)

Step 1: To a solution of (R)-4-bromo-2-[[(tert-butoxy)carbonyl]amino]butanoic acid tert-butyl ester (300 mg, 0.843 mmol) in DMF (2 mL) at RT is added phenol (0.0786 mL, 0.885 mmol) and K₂CO₃ (349 mg, 2.53 mmol). The RM is stirred at 60° C. RT for 1 hr. Water and EtOAc are added and the 2 phases are separated. The inorg. layer is extracted further with EtOAc (2×). The combined org. layers are dried over Na₂SO₄, filtered and concentrated. Crude is absorbed over isolute and the product is purified by FC (Heptane/EtOAc, from 100/0 to 50/50) to give tert-butyl N-(tert-butoxycarbonyl)-O-phenyl-D-homoserinate as a light-yellow oil (267 mg). LC-MS B: t_{R}=1.08 min; [M+H]⁺=352.32.

Step 2: Sodium hydride (60% dispersion in mineral oil) (43.7 mg, 1.14 mmol) is added portionwise to a RT solution of tert-butyl N-(tert-butoxycarbonyl)-O-phenyl-D-homoserinate (267 mg, 0.76 mmol) and iodomethane (0.0717 mL, 1.14 mmol) in DMF (5 mL) under argon and the resulting mixture is stirred for 1h. The RM is partioned between water and EtOAc. The layers are separated, and the aq. phase is re-extracted with EtOAc (2×). The combined org. extracts are washed with water and brine, dried over Na₂SO₄, filtered and evaporated in vacuo to give tert-butyl N-(tert-butoxycarbonyl)-N-methyl-O-phenyl-D-homoserinate (226 mg) as a pale orange oil. No purification at this step. LC-MS B: t_{R}=1.13 min; [M+H]⁺=366.31.

Step 3: TFA (0.474 mL, 6.18 mmol) is added to a RT solution of tert-butyl N-(tert-butoxycarbonyl)-N-methyl-O-phenyl-D-homoserinate (226 mg, 0.618 mmol) in DCM (5 mL) and the resulting mixture is stirred at RT for 6h. The volatiles are removed extensively in vacuo; the resulting residue is dissolved back in DCM to repeat the co-evaporation process 2 further times to give the crude product (266 mg) as the (1:1) 2,2,2-trifluoroacetate salt of (R)-1-carboxy-N-methyl-3-phenoxypropan-1-aminium, as a brown oil which is used as such in the next step. LC-MS B: t_{R}=0.44 min; [2M+H]⁺=419.36.

Step 4: Thionyl chloride (0.375 mL, 5.08 mmol) is added at rt to a solution of (R)-1-carboxy-N-methyl-3-phenoxypropan-1-aminium 2,2,2-trifluoroacetate (266 mg, 1.27 mmol) in MeOH (5 mL). The RM is stirred at 60° C. for 16 h. The mixture is poured into ice water and extracted with DCM (3×). The combined org. layers are washed with brine, dried over MgSO₄, filtered and concentrated under reduced pressure to give the crude methyl N-methyl-O-phenyl-D-homoserinate (101 mg) as a colorless oil. No purification at this stage. LC-MS B: t_{R}=0.54 min; [M+H]⁺=224.31.

Step 5&6: The title compound is prepared as a colorless oil from Boc-N-methyl-L-leucine and methyl N-methyl-O-phenyl-D-homoserinate following the sequence of reactions described for C-2.1, step 1 and 2. LC-MS B: t_{R}=1.03 min; [M+H]⁺=437.31.

### General Method 28 for the Synthesis of Building Blocks C

### (RS)—N—(N-(tert-Butoxycarbonyl)-N-methyl-L-leucyl)-N-methyl-O-(5-methylpyrazin-2-yl)homoserine (C-28.1)

Step 1-3: The sequence of the 3 first reactions described for C-18.2 gives the key intermediate methyl O-benzyl-N—(N-(tert-butoxycarbonyl)-N-methyl-L-leucyl)-N-methyl-D-homoserinate as a colorless oil after purification by FC (0 to 100% of EtOAc in Heptane). LC-MS B: t_{R}=1.12 min; [M+H]⁺=465.38.

Step 4: Under N₂, methyl O-benzyl-N—(N-(tert-butoxycarbonyl)-N-methyl-L-leucyl)-N-methyl-D-homoserinate (5500 mg, 11.8 mmol) is dissolved in MeOH (100 mL); the vessel is purged with N₂/vacuum (3×) before Pd(OH)₂/C (20 wt. %) (830 mg) is added. After inertising another three times a H₂ balloon is connected and the RM is stirred for 16 h under H₂ atmosphere. The heterogeneous RM is filtered over a glass fiber filter (washing with methanol/THF). The filtrate is then concentrated to dryness under reduced pressure to yield 4.36 g of methyl N—(N-(tert-butoxycarbonyl)-N-methyl-L-leucyl)-N-methyl-D-homoserinate as a colorless oil which is used as such in the next step. LC-MS B: t_{R}=0.87 min; [M+H]⁺=375.07.

Step 5: DIAD (0.194 mL, 0.966 mmol) is added dropwise to an ice-chilled suspension of 2-hydroxy-5-methylpyrazine (80 mg, 0.69 mmol), methyl N—(N-(tert-butoxycarbonyl)-N-methyl-L-leucyl)-N-methyl-D-homoserinate (271 mg, 0.725 mmol), NEt3 (0.0962 mL, 0.69 mmol) and polymer supported triphenylphosphine (1.32 mmol/g) (787 mg, 1.04 mmol) in THF (30 mL). The resulting RM is stirred at RT for 1h. The polymer is filtered off. Water is added to the filtrate and the resulting inorg. layer is extracted with EtO2 (3×). The combined org. extracts are washed with brine, dried over MgSO₄, filtered and evaporated in vacuo. Purification by FC (50% to 100% EtOAc in Heptane) yields a mixture of epimers methyl (RS)—N—(N-(tert-butoxycarbonyl)-N-methyl-L-leucyl)-N-methyl-O-(5-methylpyrazin-2-yl)homoserinate (376 mg) as a white solid. LC-MS B: t_{R}=1.07 & 1.08 min; [M+H]⁺=467.47.

Step 6: To the mixture of epimers methyl (RS)—N—(N-(tert-butoxycarbonyl)-N-methyl-L-leucyl)-N-methyl-O-(5-methylpyrazin-2-yl)homoserinate (376 mg, 0.806 mmol) in dioxane (10 mL) is added NaOH 1M (1.6 mL, 1.61 mmol). The RM is stirred at 50° C. for 1 h; it is then left returning to RT and is treated with 0.5 ml of saturated NH₄Cl solution. The resulting mixture is concentrated to dryness. The crude residue is partitioned between DCM and Water and the DCM layer is collected. The inorg. layer is acidified with a few drops of 2N aq. HCl solution (down to pH ˜ 3) and is extracted with DCM (3×). The combined org. layers are washed with acidified brine, dried over MgSO₄ and concentrated under reduced pressure to yield the title compound (RS)—N—(N-(tert-butoxycarbonyl)-N-methyl-L-leucyl)-N-methyl-O-(5-methylpyrazin-2-yl)homoserine (C-28.1) as a colorless oil (334 mg) which is used as such in the next steps. LC-MS B: t_{R}=0.94 & 0.95 min; [M+H]⁺=453.47.

### (RS)—N—(N-(tert-Butoxycarbonyl)-N-methyl-L-leucyl)-O-(2-methoxypyridin-4-yl)-N-methylhomoserine (C-28.2)

The title compound is prepared following the 6-step sequence described for C-28.1, using in step 5 commercially available 2-methoxy-4-pyridinol instead of 2-hydroxy-5-methylpyrazine, yielding C-28.2 as a colorless oil of a mixture of indistinguishable epimers. LC-MS B: t_{R}=0.76 min; [M+H]⁺=468.47.

### General Method 29 for the Synthesis of Building Blocks C

### (R)-2-((S)-2-((tert-Butoxycarbonyl)(methyl)amino)-N,4-dimethylpentanamido)-3,3-dimethylbutanoic acid (C-29.1)

Step 1: To a pale-yellow solution of 3-methyl-D-valine methyl ester (1080 mg, 7.14 mmol) in dioxane (20 mL) is added NaOH 1 M (14.3 mL, 14.3 mmol). The resulting mixture is then treated with Boc₂O (1.84 mL, 7.85 mmol) and stirred at room temperature for 24h. The mixture is concentrated under reduced pressure and the residue is partitioned between sat. aq. NH₄Cl solution and DCM. The inorg. layer is extracted further with DCM (2×) and the combined org. extracts are dried over MgSO₄ and concentrated under reduced pressure. The crude residue is redissolved in MeCN and washed with Heptane then coevaporated with Et₂O to afford the methyl (R)-2-((tert-butoxycarbonyl)amino)-3,3-dimethylbutanoate as a colorless oil (1.08 g). No further purification at this stage. LC-MS B: t_{R}=0.92 min; [M+H]⁺=246.22 & [2M+H]⁺=491.27.

Step 2: Sodium hydride (60% dispersion in mineral oil) (253 mg, 6.6 mmol) is added portionwise, at RT, under argon, to a solution of methyl (R)-2-((tert-butoxycarbonyl)amino)-3,3-dimethylbutanoate (1080 mg, 4.4 mmol) and iodomethane (0.415 mL, 6.6 mmol) in DMF (17 mL). The resulting mixture is stirred at RT for 1h. The RM is quenched by careful addition of sat. aq. NH₄Cl and the aq. layer is extracted with EtOAc (3×). The combined org. extracts are washed with a sat. aq. thiosulfate solution then with brine, dried over MgSO₄, filtered and evaporated under reduced pressure to give methyl (R)-2-((tert-butoxycarbonyl)(methyl)amino)-3,3-dimethylbutanoate (1.58 g) as a yellowish liquid which is used as such in the next step. LC-MS B: t_{R}=1.01 min; [M+H]⁺=260.23.

Step 3-5: The title compound is prepared as an off-white solid from Boc-N-methyl-L-leucine and methyl (R)-2-((tert-butoxycarbonyl)(methyl)amino)-3,3-dimethylbutanoate following the sequence of reactions described for C-18.1, step 2 to 4; Purification by FC (0% to 100% EtOAc in Heptane, monitoring with ELSD). LC-MS B: t_{R}=1.02 min; [M+H]⁺=373.32.

### (R)-2-((S)-2-((tert-Butoxycarbonyl)(methyl)amino)-N,4-dimethylpentanamido)-4-phenylbutanoic acid (C-29.2)

The title compound is prepared following the 5-step sequence described for C-29.1, starting in Step 1 from commercially available ethyl (R)-2-amino-4-phenylbutanoate hydrochloride instead of 3-methyl-D-valine methyl ester, yielding C-29.2 as a yellow oil. LC-MS B: t_{R}=1.03 min; [M+H]⁺=421.38.

### Building Blocks D

### O-Benzyl-N-(tert-Butoxycarbonyl)-N-methylserine (D1-1.1)

Step 1: Paraformaldehyde (579 mg, 4.5 mmol) is added to a RT soln. of Boc-D-Ser-(Bzl)-OH (271 mg, 0.9 mmol) and pTsOH (15.8 mg, 0.09 mmol) in PhMe (9 mL) and the RM is refluxed in a Dean Stark apparatus overnight. The mix. is washed with aq. NaHCO₃, the org. layer is separated, dried (MgSO₄) and concentrated under reduced pressure. Purification by FC (eluting from 0% to 70% EtOAc in hept) yields tert-butyl (R)-4-((benzyloxy)methyl)-5-oxooxazolidine-3-carboxylate (178 mg, 65%) as a white solid. LC-MS B: t_{R}=0.98 min; [M+H]⁺=308.09.

Step 2: TFA (2.0 mL, 26.1 mmol) is added to a RT soln. of tert-butyl (R)-4-((benzyloxy)methyl)-5-oxooxazolidine-3-carboxylate (179 mg, 0.581 mmol) and triethylsilane (0.5 mL, 3.1 mmol) in CHCl₃ (3 mL) and the RM is stirred at RT for 3 h. The mix. is concentrated, and the residue is re-dissolved in TBME and extracted with aq. NaHCO₃. The aq. layer is acidified with 1 N HCl to pH 3 and extracted with TBME. The org. layer is concentrated to yield O-benzyl-N-methyl-DL-serine as a white solid which is used as such in the next step. LC-MS B: t_{R}=0.43 min; [M+H]⁺=210.34.

Step 3: Di-tert-butyl dicarbonate (128 mg, 0.581 mmol) is added to a RT soln. of O-benzyl-N-methyl-D-serine (122 mg, 0.581 mmol) and TEA (0.404 mL, 2.9 mmol) in THF (6 mL) and the RM is stirred at RT overnight. The mix. is concentrated and to the residue is added TBME and water. The layers are separated and the aq. layer is acidified (pH 5) with 10% citric acid. The aq. layer is extracted with TBME (3×) and the combined org. layers are dried (MgSO₄), filtered, and concentrated to yield the title compound D1-1.1 (136 mg, 76%) as a viscous yellow oil. LC-MS B: t_{R}=0.88 min; [M+H]⁺=310.17. Whether racemization occurs during this synthetic step or in an earlier step was not determined. The racemic mix. is used as such in the next step.

Listed in Table D1-1 below are building blocks D1 that are prepared from the corresponding starting materials in analogy to the sequence described above for D1-1.1.

TABLE D1-1

| | | | t_{R} [min] | MS-data |
|---|---|---|---|---|
| | | | LC-MS | m/z |
| No. | SM | Product | Method | [M + H]⁺ |
| D1-1.2 | Boc-D-Ser- | N-(tert-Butoxycarbonyl)-O-(tert-butyl)-N- | 0.85 | 276.22 |
| | (tBu)-OH | methylserine | B | |
| | | Racemisation occurred | | |

### N-(((9H-Fluoren-9-yl)oxy)carbonyl)-N-(3,3-dimethylbutyl)glycine (D1-2.1)

Step 1: In a Dean-Stark apparatus, pTsOH (65.3 mg, 0.336 mmol) is added to a RT suspension of Fmoc-Gly-OH (1.0 g, 3.36 mmol) and 3,3-dimethylbutyraldehyde (0.489 mL, 3.7 mmol) in PhMe (60 mL) and the resulting turbid mix. is refluxed at 110° C. for 4 h. After cooling to RT the RM is diluted with EtOAc and the product is washed with sat. aq. NaHCO₃ (2×). The org. layer is dried (MgSO₄), filtered, and concentrated to give (9H-fluoren-9-yl)methyl 2-neopentyl-5-oxooxazolidine-3-carboxylate as a light yellow solid (0.99 g, 78%) which is used as such in the next step. LC-MS B: t_{R}=1.13 min; [M+H]⁺=380.19.

Step 2: TFA (4.94 mL, 64.6 mmol) is added to a RT soln. of (9H-fluoren-9-yl)methyl 2-neopentyl-5-oxooxazolidine-3-carboxylate (980 mg, 2.58 mmol) and triethylsilane (1.25 mL, 7.75 mmol) in DCM (13 mL) and the resulting mix. is stirred at RT overnight. The RM is diluted with DCM and washed with water (2×). The org. layer is dried (MgSO₄), filtered, and concentrated. Purification by FC (eluting with 0% to 50% EtOAc in hept and 0.5% AcOH) yields the title compound D1-2.1 (866 mg, 88%) as a colourless solid. LC-MS B: t_{R}=1.05 min; [M+H]⁺=382.18.

Listed in Table D1-2 below are building blocks D (Fmoc-protected) that are prepared from the corresponding starting material in analogy to the sequence described above for D1-2.1. In Step 2, in some cases the triethylsilane is omitted.

TABLE D1-2

| | | | t_{R} [min] | MS-data |
|---|---|---|---|---|
| | | | LC-MS | m/z |
| No. | SM | Product | Method | [M + H]⁺ |
| D1-2.2 | 2- | N-(((9H-Fluoren-9-yl)methoxy)carbonyl)- | 1.10 | 408.23 |
| | Cyclohexyl- | N-(2-cyclohexylethyl)glycine | B | |
| | acetaldehyde | | | |
| D1-2.3 | Paraformalde- | (S)-2-((((9H-Fluoren-9- | 1.04 | 380.40 |
| | hyde | yl)methoxy)carbonyl)(methyl)amino)-3- | B | |
| | | cyclobutylpropanoic acid | | |

### N-(((9H-Fluoren-9-yl)methoxy)carbonyl)-N-phenethylglycine (D1-2.4)

Step 1: TEA (1.67 mL, 12 mmol) is added to a RT soln. of phenylacetaldehyde (0.78 mL, 6 mmol) and glycine tert-butyl ester HCl-salt (1.0 g, 6 mmol) in MeOH (40 mL). After 1 h at RT, NaBH(OAc)₃ (2.68 g, 12 mmol) is added and the RM is stirred at RT overnight. The RM is filtered, concentrated and partitioned between EtOAc and aq. NaHCO₃. The org. layer is separated and the aq. layer is extracted with EtOAc. The combined org. layers are washed with brine, dried (MgSO₄), filtered, and concentrated. Purification by FC (eluting with 50% EtOAc in hept) yields tert-butyl phenethylglycinate (0.193 g, 14%) as a colourless oil. LC-MS D t_{R}=0.63 min; [M+H]⁺=236.45.

Step 2: N-(9-Fluorenylmethoxycarbonyloxy)succinimide (417 mg, 1.23 mmol) is added to a RT suspension of tert-butyl phenethylglycinate (193 mg, 0.82 mmol) and sodium carbonate (261 mg, 2.46 mmol) in dioxane (5 mL) and water (3 mL) and the RM is stirred at RT overnight. The dioxane is evaporated and the residue is partitioned between EtOAc and water. The org. layer is dried (MgSO₄), filtered, and concentrated to give tert-butyl N-(((9H-fluoren-9-yl)methoxy)carbonyl)-N-phenethylglycinate (0.484 g, 129%) which is used as such in the next step. LC-MS D t_{R}=1.34 min; [M+H]⁺=458.29.

Step 3: TFA (0.4 mL, 5.05 mmol) is added to a 0° C. soln. of tert-butyl N-(((9H-fluoren-9-yl)methoxy)carbonyl)-N-phenethylglycinate (484 mg, 0.505 mmol) in DCM (2 mL) and the resulting RM is stirred at RT overnight. The mix. is concentrated and the crude product is purified by FC (eluting with DCM/MeOH 19:1) to give the title compound D1-2.4 (0.266 g, 131%) as a colourless oil. LC-MS D t_{R}=1.09 min; [M+H]⁺=402.17.

Listed in Table D1-2 below are building blocks D (Fmoc-protected) that are prepared from the corresponding starting material in analogy to the sequence described above for D1-2.4.

TABLE D1-2

| | | | t_{R} [min] | MS-data |
|---|---|---|---|---|
| | | | LC-MS | m/z |
| No. | SM | Product | Method | [M + H]⁺ |
| D1-2.5 | 3- | N-(((9H-Fluoren-9-yl)methoxy)carbonyl)- | 1.11 | 446.30. |
| | (benzyloxy)propanal | N-(3-(benzyloxy)propyl)glycine | B | |
| D1-2.6 | 2-(oxan-4- | N-(((9H-Fluoren-9-yl)methoxy)carbonyl)- | 0.95 | 410.43 |
| | yl)acetaldehyde | N-(2-(tetrahydro-2H-pyran-4- | D | |
| | | yl)ethyl)glycine | | |

### 1-((tert-Butoxycarbonyl)(methyl)amino)cyclobutane-1-carboxylic acid is commercially available and abbreviated with D1-3.0

### 2-((tert-Butoxycarbonyl)(methyl)amino)-3-cyclopentylpropanoic acid (D1-4.1)

NaH (187 mg, 4.88 mmol) is added to a 0° C. soln. of 2-{[(tert-butoxy)carbonyl]amino}-3-cyclopentylpropanoic acid (432 mg, 1.63 mmol) in THF (8 mL), then Mel (0.51 mL, 8.13 mmol) is added and the RM is allowed to reach RT while stirring for 10 h. The RM is quenched with H₂O and extracted with EtOAc to yield the title compound D1-4.1 (318 mg, 72%) which is used as such in the next step. LC-MS B: t_{R}=0.94 min; [M+H]⁺=272.30.

Listed in Table D1-4 below are building blocks D1 that are prepared from the corresponding starting materials in analogy to the method described above for D1-4.1.

TABLE D1-4

| | | | t_{R} [min] | MS-data |
|---|---|---|---|---|
| | | | LC-MS | m/z |
| No. | SM | Product | Method | [M + H]⁺ |
| D1-4.2 | (R)-2-((tert- | (R)-2-((tert- | 0.51 | 330.23 |
| | Butoxycarbonyl)amino)-3- | Butoxycarbonyl)(methyl)amino)-3- | I | |
| | (naphthalen-1-yl)propanoic acid | (naphthalen-1-yl)propanoic acid | | |
| D1-4.3 | (tert-Butoxycarbonyl)-D-valine | N-(tert-Butoxycarbonyl)-N-methyl-D- | 0.39 | 232.30 |
| | | valine | I | |
| D1-4.4 | (tert-Butoxycarbonyl)-D-leucine | N-(tert-Butoxycarbonyl)-N-methyl-D- | 0.45 | 246.31 |
| | | leucine | I | |
| D1-4.5 | (R)-2-((tert- | (R)-2-((tert- | 0.57 | 286.34 |
| | Butoxycarbonyl)amino)-3- | Butoxycarbonyl)(methyl)amino)-3- | I | |
| | cyclohexylpropanoic acid | cyclohexylpropanoic acid | | |
| D1-4.6 | 2-((tert-Butoxycarbonyl)amino)- | 2-((tert- | 0.37 | No mass |
| | 4,4-difluorobutanoic acid | Butoxycarbonyl)(methyl)amino)-4,4- | I | |
| | | difluorobutanoic acid | | |

### (S)-2-(tert-Butoxycarbonyl-methyl-amino)-3-cyclopentyl-propionic acid (02-1.1)

NaH (914 mg, 22.9 mmol) is added to a 0° C. sol. of (S)-2-((tert-butoxycarbonyl)amino)-3-cyclopentylpropanoic acid (2.0 g, 7.62 mmol) in THF (32 mL). After 5 min, Mel (1.9 mL, 30.5 mmol) is added, the ice bath removed and the mix. stirred at RT overnight. The reaction is diluted with DCM (100 mL) and quenched with 2 M aq. HCl (27 mL→pH=1). The layers are separated and the aq. layer is extracted with DCM (2×100 mL). The combined org. layers are washed with brine (100 mL), dried (MgSO₄), filtered, and concentrated to yield the title compound (2.28 g, 110%) as an orange oil. LC-MS B: t_{R}=0.94 m2; [M+H]⁺=272.30.

Listed in Table D2-1 below are building blocks 02 that are prepared from the corresponding starting materials in analogy to the method described above for 02-1.1.

TABLE D2-1

| | | | t_{R} [min] | MS-data |
|---|---|---|---|---|
| | | | LC-MS | m/z |
| No. | SM | Product | Method | [M + H]⁺ |
| D2-1.2 | Boc-beta-Cyclohexyl-L-alanine | (S)-2-(tert-Butoxycarbonyl-methyl- | 0.98 | 286.31 |
| | monohydrate | amino)-3-cyclohexyl-propionic acid | B | |
| D2-1.3 | (2S)-2-{[(tert- | (S)-2-((tert- | 0.94 | 272.33. |
| | Butoxy)carbonyl]amino}-3-cyclo | Butoxycarbonyl)(methyl)amino)pent- | B | |
| | pentylpropanoic acid | 4-ynoic acid | | |
| D2-1.4 | (S)-2-(Boc-Amino)-3- | (S)-2-((tert- | 0.81 | 244.34 |
| | cyclopropylpropanoic acid | Butoxycarbonyl)(methyl)amino)-3- | B | |
| | | cyclopropylpropanoic acid | | |

### (S)-2-((tert-Butoxycarbonyl)(methyl)amino)-3-cyclobutylpropanoic acid (D2-2.1)

Step 1: Citric acid is added to a RT soln. of Boc-Ala(beta-cyclobutyl)-OH diisopropylamine (1.0 g, 2.84 mmol) in H₂O to lower the pH to 4, then the free carboxylic acid is extracted with DCM (3×) to yield Boc-Ala(beta-cyclobutyl)-OH (692 mg, 2.84 mmol) which is used as such in the next step.

Step 2: NaH (60% dispersion in mineral oil, 341 mg, 8.53 mmol) is added to a 000 soln. of Boc-Ala(beta-cyclobutyl)-OH (692 mg, 2.84 mmol) in THF (10 mL) followed by the dropwise addition of Mel (1.43 mL, 22.8 mmol). The resulting mix. is stirred at RT for 18 h. The RM is quenched with water and extracted with TBME (3×). The aq. layer is acidified with citric acid to pH 4 and extracted with EtOAc (3×). The combined EtOAc layers are dried (MgSO₄), filtered, and concentrated to yield D2-2.1 (801 mg, 109%) which is used as such in the next step. LC-MS B: t_{R}=0.89 min; [M+H]⁺=258.08.

### N-((Allyloxy)carbonyl)-N-methyl-L-leucine (D2-3.1)

Step 1: 4 M HCl in dioxane (11 mL, 44 mmol) is added to a RT soln. of Boc-N-Me-Leu-OH (2.78 g, 11 mmol) in DCM (20 mL). The mix. is stirred at RT for 2 h. The RM is concentrated to yield (S)-4-methyl-2-methylamino-pentanoic acid (2.14 g) which is used as such in the next step.

Step 2: Allyl chloroformate (1.27 mL, 11.6 mmol) is added to (S)-4-methyl-2-methylamino-pentanoic acid (1.59 g, 11 mmol) and Na₂CO₃ (4.08 g, 38.5 mmol) in dioxane/H₂O 3/5 (48 mL). The mix. is stirred at RT overnight, then the RM is diluted with EtOAc and acidified to pH 2 using 2 M aq. HCl. The layers are separated and the aq. layer is extracted with EtOAc (2×). The combined org. layers are dried (Na₂SO₄), filtered, and concentrated to yield D2-3.1 (2.5 g, 99%) which is used as such in the next step. LC-MS B: t_{R}=0.80 min; [M+H]⁺=230.43.

### Intermediates

### Benzyl (2-(tert-butoxy)ethyl)glycinate (IM-1.1)

A soln. of benzyl bromoacetate (0.165 mL, 1 mmol) in MeCN (2 mL) is added dropwise to a RT soln. of 2-(tert-butoxy)ethan-1-amine (493 mg, 4 mmol) in MeCN (3 mL) and the RM is stirred at RT for 50 min. The precipitate is filtered off and the filtrate is directly purified by prep. HPLC (basic) to yield IM-1.1 (214 mg, 81%) as a colourless oil. LC-MS I: t_{R}=0.93 min; [M+H]⁺=266.25.

Listed in Table IM-1 below are Intermediates that are prepared from the corresponding starting materials in analogy to the method described above for IM-1.1.

TABLE IM-1

| | | | t_{R} [min] | MS-data |
|---|---|---|---|---|
| | | | LC-MS | m/z |
| No. | SM | Product | Method | [M + H]⁺ |
| IM-1.2 | C-(3-Methyl-tetrahydro-furan-3-yl)- | Benzyl (R)-((3-methyltetrahydrofuran- | 0.82 | 264.21 |
| | methylamine | 3-yl)methyl)glycinate | I | |
| IM-1.3 | 1-(3,4-Dihydro-2H-chromen-3- | rac-Benzyl (R)-(chroman-3- | 1.03 | 312.23 |
| | yl)methanamine | ylmethyl)glycinate | I | |
| IM-1.4 | 2-{7-Oxabicyclo[2.2.1]heptan-2- | Benzyl (2-(7-oxabicyclo[2.2.1]heptan- | 0.82 | 289.97 |
| | yl}ethan-1-amine | 2-yl)ethyl)glycinate | I | |
| IM-1.5 | 2-{5-Oxaspiro[3.4]octan-6-yl}ethan- | Benzyl (2-(5-oxaspiro[3.4]octan-6- | 0.99 | 304.21 |
| | 1-amine | yl)ethyl)glycinate | I | |
| IM-1.6 | 2-(Oxolan-2-yl)ethan-1-amine | Benzyl (2-(tetrahydrofuran-2- | 0.81 | 264.23 |
| | | yl)ethyl)glycinate | I | |
| IM-1.7 | 2-(Oxan-3-yl)ethan-1-amine | Benzyl (2-(tetrahydro-2H-pyran-3- | 0.85 | 278.24 |
| | | yl)ethyl)glycinate | I | |
| IM-1.8 | 2-{2,9-Dioxaspiro[5.5]undecan-3- | Benzyl (2-(2,9- | 0.87 | 348.22 |
| | yl}ethan-1-amine | dioxaspiro[5.5]undecan-3- | I | |
| | | yl)ethyl)glycinate | | |
| IM-1.9 | 1,4-Dioxane-2-methanamine•HCl | Benzyl ((1,4-dioxan-2- | 0.71 | 266.16 |
| | | yl)methyl)glycinate | I | |
| IM-1.10 | 2-(Tetrahydropyran-2- | Benzyl (2-(tetrahydro-2H-pyran-2- | 0.90 | 278.21 |
| | yl)ethylamine•HCl | yl)ethyl)glycinate | I | |
| IM-1.11 | 2-(3-Methoxycyclobutyl)ethan-1- | Benzyl (2-(3- | 0.88 | 278.20 |
| | amine | methoxycyclobutyl)ethyl)glycinate | I | |
| IM-1.12 | 2-Cyclohexylethylamine | Benzyl (2-cyclohexylethyl)glycinate | 1.19 | 276.26 |
| | | | I | |
| IM-1.13 | (Tetrahydro-2H-pyran-3- | Benzyl ((tetrahydro-2H-pyran-3- | 0.81 | 264.33 |
| | yl)methanamine | yl)methyl)glycinate | I | |
| IM-1.14 | (6,6-Dimethyltetrahydro-2H-pyran-2- | [(6,6-Dimethyl-tetrahydro-pyran-2- | 1.02 | 292.32 |
| | yl)methanamine | ylmethyl)-amino]-acetic acid benzyl | I | |
| | | ester | | |
| IM-1.15 | AM-1.72 | Benzylbenzyl (3- | 2.18 | 284.2 |
| | | phenylpropyl)glycinate | I | |
| IM-1.16 | AM-1.75 | Benzylbenzyl ((2,3- | 0.99 | 298.29 |
| | | dihydrobenzofuran-2- | I | |
| | | yl)methyl)glycinate | | |

### Benzyl (S)-((tetrahydro-2H-pyran-2-yl)methyl)glycinate (IM-2.1)

Step 1: NaH (60% dispersion in mineral oil, 2.41 g, 60.1 mmol) is added to a 000 soln. of tetrahydropyran-2-methanol (6.0 g, 50.1 mmol) in THF (300 mL). The RM is warmed to RT and after stirring for 30 min benzyl bromide (7.29 mL, 60.1 mmol) is added and the mix. is stirred at RT overnight. Sat aq. NH₄Cl is added and the mix. is extracted with EtOAc (2×). The combined org. layers are dried (MgSO₄), filtered, and concentrated. Purification by FC (hept/EtOAc 1:0 to 9:1) yields rac-2-((benzyloxy)methyl)tetrahydro-2H-pyran (10.3 g, 100%) as a colourless oil.

Step 2: Chiral separation yields (R)-2-((benzyloxy)methyl)tetrahydro-2H-pyran and (S)-2-((benzyloxy)methyl)tetrahydro-2H-pyran which were used as such in the next step. The configuration is assigned by optical rotation.

Step 3: 10% Pd/C (2.54 g, 2.39 mmol) is added to a RT soln. of (S)-2-((benzyloxy)methyl)tetrahydro-2H-pyran (4.92 g, 23.9 mmol) in MeOH (100 mL) and the RM is stirred at RT for 3 h under a H₂ atm. The RM is filtered, and the filtrate is concentrated under reduced pressure. Purification by Kugelrohrdestillation yields (S)-(tetrahydro-2H-pyran-2-yl)methanol (2.54 g, 92%) as a colourless oil.

Step 4: 2-Nitrobenzenesulfonyl chloride (7.54 g, 33 mmol) is added portionwise to a 0° C. soln. of H-Gly-OBzl HCl (6.11 g, 30 mmol) and TEA (8.77 mL, 63 mmol) in DCM (45 mL). The mix. is warmed to RT and stirred for 1 h. The RM is concentrated, then EtOAc and water are added to the residue. The layers are separated and the aq. layer is extracted with EtOAc. The combined org. layers are dried (MgSO₄), filtered, and concentrated. Purification by FC (hept/EtOAc 0:1 to 1:1) yields benzyl ((2-nitrophenyl)sulfonyl)glycinate (10.56 g, 100%) as a white solid. LC-MS B: t_{R}=0.93 min; [M+H]⁺=351.26.

Step 5: DIAD (1 mL, 5 mmol) is added to a RT soln. of benzyl ((2-nitrophenyl)sulfonyl)glycinate (1.75 g, 5 mmol), (S)-2-((benzyloxy)methyl)tetrahydro-2H-pyran (639 mg, 5.5 mmol), and PPh₃ (1.66 g, 6 mmol) in THF (50 mL) and the RM is stirred for 1 h. The RM is concentrated and directly purified by prep. HPLC (basic) to yield (S)—N-(2-(N-(2-(benzyloxy)-2-oxoethyl)-N-((tetrahydro-2H-pyran-2-yl)methyl)sulfamoyl)phenyl)-N-oxohydroxylammonium (2.1 g, 94%) as a pale brownish oil. LC-MS I: t_{R}=1.11 min; [M+H]⁺=449.12.

Step 6: Thiophenol (0.681 mL, 6.56 mmol) is added to a RT suspension of (S)—N-(2-(N-(2-(benzyloxy)-2-oxoethyl)-N-((tetrahydro-2H-pyran-2-yl)methyl)sulfamoyl)phenyl)-N-oxohydroxylammonium (2.10 g, 4.69 mmol) and K₂CO₃ (992 mg, 7.03 mmol) in DMF (25 mL). The RM is stirred at RT for 1 h, then the solids are filtered off and the filtrate is concentrated and purified by prep. HPLC (basic) to yield IM-2.1 (976 mg, 79%) as a colourless oil. LC-MS I: t_{R}=0.88 min; [M+H]⁺=264.22.

Listed in Table IM-2 below are Intermediates that are prepared in analogy to the sequence described for IM-2.1.

TABLE IM-2

| | | | t_{R} [min] | MS-data |
|---|---|---|---|---|
| | | | LC-MS | m/z |
| No. | SM | Product | Method | [M + H]⁺ |
| IM-2.2 | (R)-(Tetrahydro-2H-pyran-2-yl)methanol | Benzyl (R)-((tetrahydro- | 0.88 | 264.21 |
| | (see IM-2.1, steps 1 to 3) | 2H-pyran-2- | I | |
| | | yl)methyl)glycinate | | |
| IM-2.3 | (S)-2-(Tetrahydro-2H-pyran-2-yl)ethan-1-ol | Benzyl (S)-(2- | 0.90 | 278.24 |
| | (synthesised in analogy to IM-2.1, steps 1 to 3) | (tetrahydro-2H-pyran-2- | I | |
| | | yl)ethyl)glycinate | | |
| IM-2.4 | (R)-2-(Tetrahydro-2H-pyran-2-yl)ethan-1-ol | Benzyl (R)-(2- | 0.90 | 278.25 |
| | (synthesised in analogy to IM-2.1, steps 1 to 3 | (tetrahydro-2H-pyran-2- | I | |
| | | yl)ethyl)glycinate | | |
| IM-2.5 | (S)-Chroman-3-ylmethanol | Benzyl (S)-(chroman-3- | 1.05 | 312.23 |
| | | ylmethyl)glycinate | I | |
| IM-2.6 | (R)-Chroman-3-ylmethanol | Benzyl (R)-(chroman-3- | 1.05 | 312.25 |
| | | ylmethyl)glycinate | I | |

### Methyl methyl-D-phenylalaninate (IM-3.1)

Step 1: NaH (60% dispersion in mineral oil, 265 mg, 6.92 mmol) is added to a 0° C. soln. of Boc-D-Phe-OH (458 mg, 1.73 mmol) in DMF (8 mL), then Mel (0.05 mL, 0.8 mmol) is added and the RM is warmed to RT overnight. The solvent is evaporated and the crude product is purified by FC to yield methyl N-(tert-butoxycarbonyl)-N-methyl-D-phenylalaninate.

Step 2: 4 M HCl in dioxane (0.24 mL, 0.682 mmol) is added to a 0° C. soln. of methyl N-(tert-butoxycarbonyl)-N-methyl-D-phenylalaninate (200 mg, 0.682 mmol) in DCM (3 mL) and the mix. is stirred at RT for 2 h. The RM is concentrated in vacuo to yield the title compound as a white solid. LC-MS B: t_{R}=0.46 min; [M+H]⁺=194.21.

Listed in Table IM-3 below are intermediates that are prepared from the corresponding starting materials in analogy to the sequence described above for IM-3.1.

TABLE IM-3

| | | | t_{R} [min] | MS-data m/z |
|---|---|---|---|---|
| No. | SM | Product | LC-MS Method | [M + H]⁺ |
| IM-3.2 | Boc-beta-Cyclohexyl-D- | Methyl (R)-3-cyclohexyl-2- | 0.58 | 200.39 |
| | alanine monohydrate | (methylamino)propanoate | B | |
| IM-3.3 | Boc-D-Cyclopentyl-glycine | Methyl (R)-2-cyclopentyl-2- | 0.45 | 172.00 |
| | | (methylamino)acetate (HCl-salt) | B | |

### 3,3-Difluoro-1-methylamino-cyclobutanecarboxylic acid methyl ester (IM-4.1)

Step 1: 2.0 M (trimethylsilyl)diazomethane in Et₂O (0.27 mL, 0.54 mmol) is added to a 0° C. soln. of 1-{[(tert-butoxy)carbonyl](methyl)amino}-3,3-difluorocyclobutane-1-carboxylic acid (100 mg, 0.36 mmol) in MeOH (1 mL). The RM is warmed to RT and stirring is continued for 6 h. The RM is concentrated and purified by FC (hept/EtOAc 4:1) to yield methyl 1-((tert-butoxycarbonyl)(methyl)amino)-3,3-difluorocyclobutane-1-carboxylate (52 mg, 52%) as a clearoil. LC-MS I: t_{R}=0.98 min; [M+H]⁺=280.25.

Step 2: The title compound IM-4.1 is prepared from methyl 1-((tert-butoxycarbonyl)(methyl)amino)-3,3-difluorocyclobutane-1-carboxylate following the procedure described in IM-3.1, step 2. LC-MS I: t_{R}=0.57 min; [M+H]⁺=180.31.

### 2((2-Nitrobenzen)sulfonamido)acetic acid (IM-5.1)

2-Nitrobenzenesulfonyl chloride (5.71 g, 25 mmol) is added portionwise to a 60° C. sal. of glycine (1.89 g, 25 mmol) and 32% aq. NaOH (5 mL, 50 mmol) in H₂O (20 mL). After the addition is complete the RM is stirred for 30 min at 60° C. before being cooled to 000. The mix. is acidified with conc. HCl to pH 1, and the formed precipitate is isolated by filtration to yield IM-5.1 (4.34 g, 67%) as a white solid. LC-MS B: t_{R}=0.6 min; [M+H]⁺=261.25.

Commercially Available Sulfonylchlorides

TABLE SCI

| Sulfonyl-chloride | Name |
|---|---|
| SCI-1 | Benzenesulfonyl chloride |
| SCI-2 | Pyridine-3-sulfonyl chloride |
| SCI-3 | Pyridine-2-sulfonyl chloride |
| SCI-4 | Pyridine-4-sulfonyl chloride |
| SCI-5 | 3-Methoxybenzenesulfonyl chloride |
| SCI-6 | 3-(Trifluoromethoxy)benzenesulfonyl chloride |
| SCI-7 | 2-Fluoro-5-methoxybenzenesulfonyl chloride |
| SCI-8 | 5-Methoxypyridine-3-sulfonyl chloride |
| SCI-9 | 4-Methyl-3,4-dihydro-2H-benzo[b][1,4]oxazine-7-sulfonyl chloride |
| SCI-10 | 2-Oxo-2,3-dihydrobenzo[d]oxazole-6-sulfonyl chloride |
| SCI-11 | Phenylmethanesulfonyl chloride |
| SCI-12 | Quinolin-7-ylmethanesulfonyl chloride |
| SCI-13 | Methanesulfonyl chloride |
| SCI-14 | 2-Methoxyethane-1-sulfonyl chloride |
| SCI-15 | 3-Methoxypropane-1-sulfonyl chloride |
| SCI-16 | 2-(Dimethylamino)ethane-1-sulfonyl chloride |
| SCI-17 | Tetrahydro-2H-pyran-4-sulfonyl chloride |
| SCI-18 | (Tetrahydro-2H-pyran-4-yl)methanesulfonyl chloride |
| SCI-19 | 2-Morpholinoethane-1-sulfonyl chloride |
| SCI-20 | 3-Morpholinopropane-1-sulfonyl chloride |

### Synthesis of Macrocyles Bearing a Carboxylic Acid Group

### (9S,13S,19aR,22R)-22-Benzyl-13-isobutyl-12-methyl-7,11,14,20-tetraoxo-7,8,9,10,11,12,13,14,17,18,19,19a,20,21,22,23-hexadecahydro-16H-pyrido[2′,1′:6,7][1]oxa[4,7,10,14]tetraazacycloheptadecino[17,16-c]quinoline-9-carboxylic acid (MC—COOH-1)

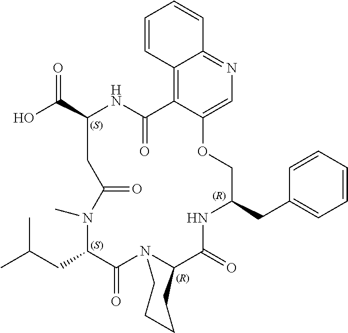

Step 1: HATU (4.12 g, 10.3 mmol) is added to a RT soln. of B-1.14 (5.0 g, 10.3 mmol), C-2.2 (3.67 g, 10.3 mmol), and DIPEA (5.29 mL, 30.9 mmol) in DMF (100 mL). The resulting mix. is stirred at RT for 10 min, then the mix. is partitioned between water and EtOAc. The layers are separated and the aq. layer is re-extracted with EtOAc (2×). The combined org. extracts are washed with brine, dried (MgSO₄), filtered, and evaporated to obtain 3-[(R)-2-({(R)-1-[(S)-2-(tert-butoxycarbonyl-methyl-amino)-4-methyl-pentanoyl]-piperidine-2-carbonyl}-amino)-3-phenyl-propoxy]-quinoline-4-carboxylic acid benzyl ester (8.78 g, 114%) as a white solid which is used as such in the next step. LC-MS I: t_{R}=1.43 min; [M+H]⁺=751.46.

Step 2: A RT soln. of 3-[(R)-2-({(R)-1-[(S)-2-(tert-butoxycarbonyl-methyl-amino)-4-methyl-pentanoyl]-piperidine-2-carbonyl}-amino)-3-phenyl-propoxy]-quinoline-4-carboxylic acid benzyl ester (8.79 g, 11.3 mmol) in EtOH (100 mL) is evacuated/purged with N₂ (3×) before Pd/C (604 mg, 0.567 mmol) is added. The RM is evacuated/purged with H₂ (3×) and stirred under a H₂ atm for 18 h. The RM is filtered and the filter cake rinsed with EtOH. The filtrate is concentrated to yield 3-[(R)-2-({(R)-1-[(S)-2-(tert-butoxycarbonyl-methyl-amino)-4-methyl-pentanoyl]-piperidine-2-carbonyl}-amino)-3-phenyl-propoxy]-quinoline-4-carboxylic acid (7.81 g, 104%) as an off-white solid which is used as such in the next step. LC-MS I: t_{R}=0.68 min; [M+H]⁺=661.20.

Step 3: Benzyl bromide (3.46 mL, 28.6 mmol) is added to a RT soln. of Fmoc-L-aspartic acid beta-tert-butyl ester (10.0 g, 23.8 mmol) and KHCO₃ (2.65 g, 26.2 mmol) in DMF (60 mL) and the resulting mix. is stirred at RT for 18 h. The mix. is concentrated and to the residue is added EtOAc and water. The layers are separated, and the aq. layer is re-extracted with EtOAc (2×). The combined org. layers are washed with water and brine, dried (MgSO₄), filtered, and concentrated to obtain (S)-2-(9H-fluoren-9-ylmethoxycarbonylamino)-succinic acid 1-benzyl ester 4-tert-butyl ester (12.21 g, 102%) as a white powder which is used as such in the next step. LC-MS B: t_{R}=1.19 min; [M+H]⁺=502.28.

Step 4: Piperidine (11.8 mL, 118 mmol) is added to a RT soln. of (S)-2-(9H-fluoren-9-ylmethoxycarbonylamino)-succinic acid 1-benzyl ester 4-tert-butyl ester (12.21 g, 23.6 mmol) in DCM (150 mL). The resulting mix. is stirred at RT for 2 h. The mix. is concentrated in vacuo at 40° C. to yield a white solid, which is triturated with hept. The solid is filtered off and purified by FC (eluting with 10% to 100% EtOAc in hept) to give (S)-2-amino-succinic acid 1-benzyl ester 4-tert-butyl ester (5.44 g, 82%) as a yellowish oil. LC-MS B: t_{R}=0.68 min; [M+H]⁺=280.36.

Steps 5: HATU (3.61 g, 9.03 mmol) is added to a RT soln. of 3-[(R)-2-({(R)-1-[(S)-2-(tert-butoxycarbonyl-methyl-amino)-4-methyl-pentanoyl]-piperidine-2-carbonyl}-amino)-3-phenyl-propoxy]-quinoline-4-carboxylic acid (6.63 g, 9.03 mmol), (S)-2-amino-succinic acid 1-benzyl ester 4-tert-butyl ester (2.55 g, 9.03 mmol), and DIPEA (4.64 mL, 27.1 mmol) in DMF (100 mL). The resulting mix. is stirred at RT for 1 h, then the mix. is partitioned between water and EtOAc. The layers are separated and the aq. re-extracted with EtOAc (2×). The combined org. extracts are washed with brine, dried (MgSO₄), filtered, and evaporated to yield (S)-2-({3-[(R)-2-({(R)-1-[(S)-2-(tert-butoxycarbonyl-methyl-amino)-4-methyl-pentanoyl]-piperidine-2-carbonyl}-amino)-3-phenyl-propoxy]-quinoline-4-carbonyl}-amino)-succinic acid 1-benzyl ester 4-tert-butyl ester (7.54 g) as a pink solid which is used as such in the next step. LC-MS I: t_{R}=1.44 min; [M+H]⁺=922.70.

Step 6: TFA (69.1 mL, 897 mmol) is added to a RT soln. of (S)-2-({3-[(R)-2-({(R)-1-[(S)-2-(tert-butoxycarbonyl-methyl-amino)-4-methyl-pentanoyl]-piperidine-2-carbonyl}-amino)-3-phenyl-propoxy]-quinoline-4-carbonyl}-amino)-succinic acid 1-benzyl ester 4-tert-butyl ester (7.54 g, 5.64 mmol) in DCM (150 mL). The resulting mix. is stirred for 3 h, then concentrated and co-evaporated with DCM (2×). The residue is taken up in DMF (100 mL), and to the soln. is added DIPEA (7.72 mL, 45.1 mmol) and HATU (2.57 g, 6.77 mmol). The mix. is stirred at RT for 1 h, then the mix. is partitioned between water and EtOAc. The layers are separated and the aq. re-extracted with EtOAc (2×). The combined org. extracts are washed with water (2×) and brine (2×), dried (MgSO₄), filtered, and evaporated. Purification by FC (eluting with 100% EtOAc) yields benzyl (9S,13S,19aR,22R)-22-benzyl-13-isobutyl-12-methyl-7,11,14,20-tetraoxo-7,8,9,10,11,12,13,14,17,18,19,19a,20,21,22,23-hexadecahydro-16H-pyrido[2′,1′:6,7][1]oxa[4,7,10,14]tetraazacycloheptadecino[17,16-c]quinoline-9-carboxylate (2.45 g, 58%) as a pink solid. LC-MS I: t_{R}=1.20 min; [M+H]⁺=748.48.

Step 7: A suspension of benzyl (9S,13S,19aR,22R)-22-benzyl-13-isobutyl-12-methyl-7,11,14,20-tetraoxo-7,8,9,10,11,12,13,14,17,18,19,19a,20,21,22,23-hexadecahydro-16H-pyrido[2′,1′:6,7][1]oxa[4,7,10,14]tetraazacycloheptadecino[17,16-c]quinoline-9-carboxylate (2.45 g, 2.95 mmol) in EtOH (25 mL) is evacuated/purged with H₂ (3×) before Pd/C (157 mg, 0.147 mmol) is added. The RM is evacuated/purged with H₂ (3×) and stirred under a H₂ atm for 24 h. The mix. is filtered and the filter cake is rinsed with EtOH. The filtrate is concentrated and purified by FC (eluting with 4% MeOH in DCM) to give the title compound (1.57 g, 81%) as an off-white solid. LC-MS I: t_{R}=0.54 min; [M+H]⁺=658.42.

Listed in Table MC—COOH-A below are carboxylic acids that are prepared from the corresponding starting materials in analogy to the 7-step sequence described above for MC—COOH-1.

TABLE MC-COOH-A

| | | | | | t_{R} [min] | MS-data |
|---|---|---|---|---|---|---|
| | | | | | LC-MS | m/z |
| No. | Structure | BB-B | BB-C | Product | Method | [M + H]⁺ |
| MC-COOH-2 | 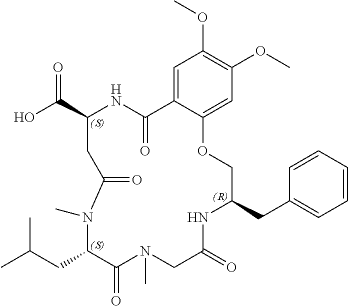 | B-1.11 | C-1.1 | (3R,9S, 13S)-3-Benzyl-9- isobutyl-17,18-dimethoxy-7,10- dimethyl-5,8,11,15-tetraoxo- 2,3,4,5,6,7,8,9,10,11,12,13,14,15- tetradecahydrobenzo[p][1]oxa [4,7,10,14] tetraazacycloheptadecine-13- carboxylic acid | 0.84 B | 627.16 |
| MC-COOH-3 | 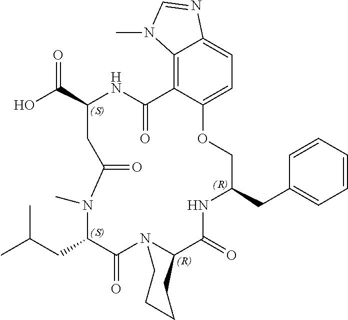 | B-2.7 | C-2.2 | (8S, 12S, 18aR,21R)-21-Benzyl- 12-isobutyl-5,11-dimethyl- 6,10,13,19-tetraoxo- 6,7,8,9,10,11,12,13,16,17,18,18a, 19,20,21,22-hexadecahydro-5H, 15H-imidazo[4′,5′:5,6]benzo [1,2-p]pyrido[2,1- f][1]oxa[4,7,10,14] tetraazacycloheptadecine-8- carboxylic acid | 0.51 I | 661.46 |
| MC-COOH-4 | 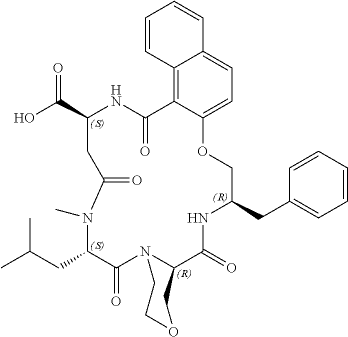 | B-1.13 | C-2.1 | (4aR,7R, 18S,22S)-7-Benzyl-22- isobutyl-21-methyl-5,16,20,23- tetraoxo- 1,2,4,4a,5,6,7,8,16,17,18,19,20, 21,22,23- hexadecahydronaphtho[1,2- p][1,4]oxazino[3,4- f][1]oxa[4,7,10,14] tetraazacycloheptadecine-18- carboxylic acid | 0.92 B | 659.25 |
| MC-COOH-5 | 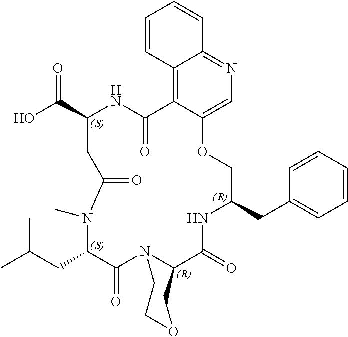 | B-1.14 | C-2.1 | (9S, 13S, 19aR,22R)-22-Benzyl- 13-isobutyl-12-methyl- 7,11,14,20-tetraoxo- 7,8,9,10,11,12,13,14,16,17,19, 19a,20,21,22,23-hexadecahydro- [1,4]oxazino[3′,4′:6,7][1]oxa[4,7, 10,14]tetraazacycloheptadecino [17,16-c]quinoline-9-carboxylic acid | 0.84 B | 660.32 |
| MC-COOH-6 | 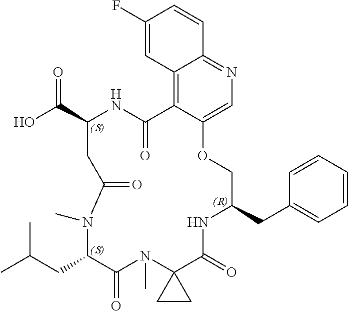 | B-1.19 | C-1.2 | (3′S,7′S, 13′R)-13′-Benzyl-20′- fluoro-7′-isobutyl-6′,9′-dimethyl- 1′,5′,8′,11′-tetraoxo-2′,3′, 4′,5′,6′,7′,8′,9′, 11′, 12′, 13′, 14′- dodecahydro-1′H- spiro[cyclopropane-1,10′- [1]oxa[4,7,10,14] tetraazacycloheptadecino[17,16- c]quinoline]-3′-carboxylic acid | 0.92 B | 662.19 |
| MC-COOH-7 | 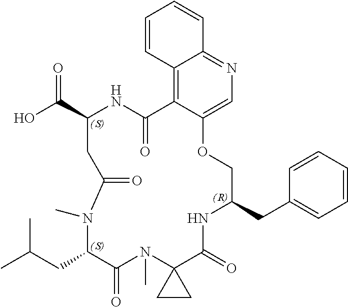 | B-1.14 | C-1.2 | (3′S,7′S, 13′R)-13′-Benzyl-7′- isobutyl-6′,9′-dimethyl-1′,5′,8′, 11′-tetraoxo-2′,3′, 4′,5′,6′,7′,8′,9′, 11′, 12′, 13′, 14′-dodecahydro- 1′H-spiro[cyclopropane-1,10′- [1]oxa[4,7,10, 14]tetraazacycloheptadecino [17,16-c]quinoline]-3′-carboxylic acid | 0.52 I | 644.27 |
| MC-COOH-15 | 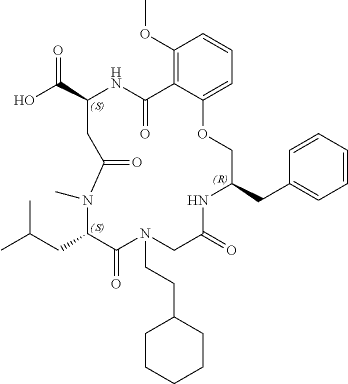 | B-1.5 | C-10.19 | (3R,9S, 13S)-3-Benzyl-7-(2- cyclohexylethyl)-9-isobutyl-16- methoxy-10-methyl-5,8,11,15- tetraoxo- 2,3,4,5,6,7,8,9,10,11,12,13, 14,15- tetradecahydrobenzo[p][1]oxa [4,7,10,14] tetraazacycloheptadecine-13- carboxylic acid | 0.66 I | 693.46 |
| MC-COOH-16 | 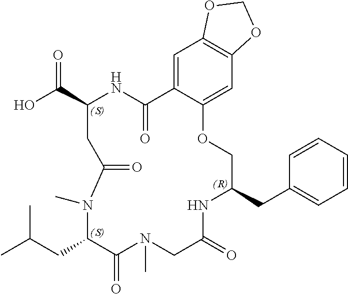 | B-1.16 | C-1.1 | (4R, 10S, 14S)-4-Benzyl-107R, 13S,17S)-7-benzyl-13-isobutyl- 8,11,14-dimethyl-6,9,12,1615,19- tetraoxo-2,20,22-trioxa- 56,7,8,9,10,11,15- tetraazatricyclo[12,13,14, 15.7.0.019,23]tetracosa-1(, 16,17),, 18,23-triene-14-19- tetradecahydro- [1,3]dioxolo[4′,5′:4,5]benzo[1,2- p][1]oxa[4,7,10,14] tetraazacycloheptadecine-17- carboxylic acid | 0.45 I | 610.87 |
| MC-COOH-24 | 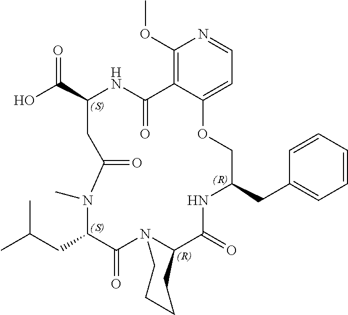 | B-1.18 | C-2.2 | (7S,11S,17aR,20R)-20-benzyl- 11-isobutyl-4-methoxy-10- methyl-5,9,12,18-tetraoxo- 5,6,7,8,9,10,11,12,15,16,17,17a, 18, 19,20,21-hexadecahydro- 14H-dipyrido[2,1-f:3′,4′- p][1]oxa[4,7,10,14] tetraazacycloheptadecine-7- carboxylic acid | 0.54 I | 638.27 |
| MC-COOH-25 | 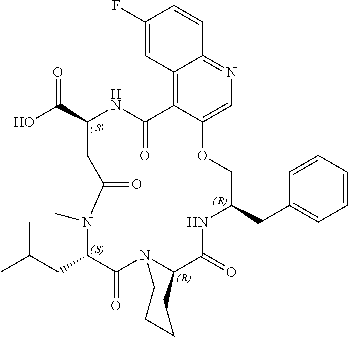 | B-1.19 | C-2.2 | (9S, 13S, 19aR,22R)-22-benzyl- 5-fluoro-13-isobutyl-12-methyl- 7,11,14,20-tetraoxo- 7,8,9,10,11,12,13,14,17,18,19,19a ,20,21,22,23-hexadecahydro- 16H- pyrido[2′,1′:6,7][1]oxa[4,7,10,14] tetraazacycloheptadecino[17,16- c]quinoline-9-carboxylic acid | 0.55 I | 676.40 |
| MC-COOH-26 | 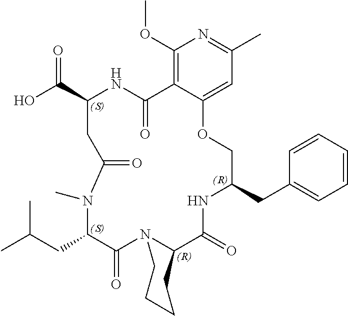 | B-4.15 | C-2.2 | (7S,11S, 17aR,20R)-20-Benzyl- 11-isobutyl-4-methoxy-2, 10- dimethyl-5,9,12,18-tetraoxo- 5,6,7,8,9,10,11,12,15,16,17,17a, 18,19,20,21-hexadecahydro-14H- dipyrido[2,1-f:3′,4′- p][1]oxa[4,7,10,14] tetraazacycloheptadecine-7- carboxylic acid | 0.52 I | 652.17 |
| MC-COOH-27 | 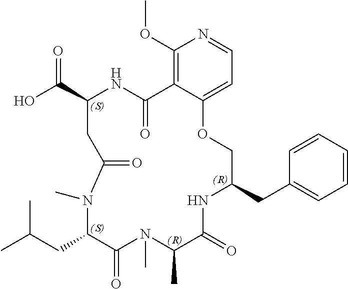 | B-1.18 | C-1.4 | (3R,6R,9S,13S)-3-benzyl-9- isobutyl-16-methoxy-6,7,10- trimethyl-5,8,11,15-tetraoxo- 2,3,4,5,6,7,8,9,10,11,12,13,14,15- tetradecahydropyrido[3,4- p][1]oxa[4,7,10,14] tetraazacycloheptadecine-13- carboxylic acid | 1.66 J | 612.20 |
| MC-COOH-28 | 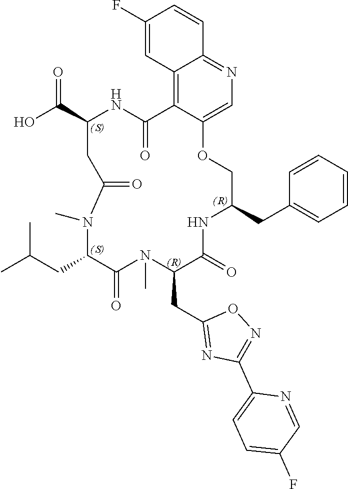 | B-1.19 | C-22.1 | (3S,7S,10R,13R)-13-Benzyl-20- fluoro-10-((3-(5-fluoropyridin-2- yl)-1,2,4-oxadiazol-5-yl)methyl)- 7-isobutyl-6,9-dimethyl-1,5,8,11- tetraoxo- 1,2,3,4,5,6,7,8,9,10,11,12,13,14- tetradecahydro-[1]oxa[4,7,10,14] tetraazacycloheptadecino[17,16- c]quinoline-3-carboxylic acid | 0.97 | 813.14 |
| MC-COOH-29 | 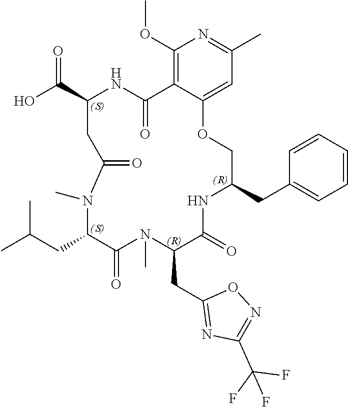 | B-4.15 | C-22.2 | (3R,6R,9S,13S)-3-Benzyl-9- isobutyl-16-methoxy-7,10,18- trimethyl-5,8,11,15-tetraoxo-6- ((3-(trifluoromethyl)-1,2,4- oxadiazol-5-yl)methyl)- 2,3,4,5,6,7,8,9,10,11,12,13,14,15- tetradecahydropyrido[3,4- p][1]oxa[4,7,10,14] tetraazacycloheptadecine-13- carboxylic acid | 0.97 | 762.31 |
| MC-COOH-30 | 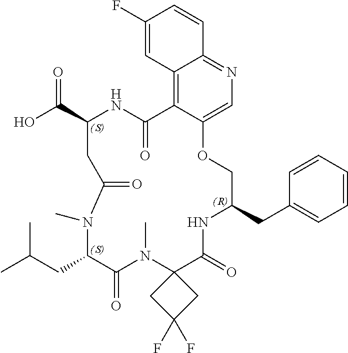 | B-1.19 | C-5.4 | (3′S,7′S, 13′R)-13′-benzyl- 3,3,20′-trifluoro-7′-isobutyl-6′,9′- dimethyl-1′,5′,8′,11′-tetraoxo- 2′,3′,4′,5′,6′,7′,8′,9′, 11′, 12′,13′, 14′-dodecahydro-1′H- spiro[cyclobutane-1,10′- [1]oxa[4,7,10,14] tetraazacycloheptadecino[17,16- c]quinoline]-3′-carboxylic acid | 0.97 B | 712.07 |

### (7R,13S,17S)-7-Benzyl-11-(3,3-dimethylbutyl)-13-isobutyl-14-methyl-9,12,15,19-tetraoxo-6,7,8,9,10,11,12,13,14,15,16,17,18,19-tetradecahydro-[1,3]dioxolo[4′,5′:4,5]benzo[1,2-p][1]oxa[4,7,10,14]tetraazacycloheptadecine-17-carboxylic acid (MC—COOH-8)

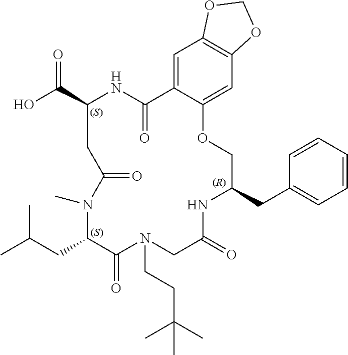

The synthesis is performed according to a general method for the solid-phase synthesis. The Wang resin (153 mg, 0.135 mmol) is swollen in DMF (5 mL) for 1 h. The solvent is drained off and the resin is treated with a soln. of Fmoc-Asp(OAII)-OH (272 mg, 0.675 mmol), TBTU (214 mg, 0.648 mmol), DIPEA (0.236 mL, 1.35 mmol) in DMF (3 mL) for 3 h. The Fmoc deprotection is performed by treating the resin (2×5 min) with a soln. of piperidine 20% in DMF (3 mL). After filtration, the resin is washed with DMF (3×4 mL) and DCM (3×4 mL). The resin is treated with a soln. of B-Acid-4 (394 mg, 0.675 mmol), TBTU (214 mg, 0.648 mmol), HOBt (103 mg, 0.675 mmol) and DIPEA (0.236 mL, 1.35 mmol) in DMF (3 mL) for 3 h. After filtration the resin is washed with DMF (3×4 mL) and DCM (3×4 mL). The Fmoc deprotection is performed by treating the resin (2×5 min) with a soln. of piperidine 20% in DMF (3 mL). After filtration the resin is washed with DMF (3×4 mL) and DCM (3×4 mL). The resin is then treated with a soln. D1-2.1 (257 mg, 0.675 mmol), TBTU (214 mg, 0.648 mmol), HOBt (103 mg, 0.675 mmol), and DIPEA (0.236 mL, 1.35 mmol) in DMF (3 mL) for 3 h. After filtration the resin is washed with DMF (3×4 mL) and DCM (3×4 mL). The Fmoc deprotection is performed by treating the resin (2×5 min) with a soln. of piperidine 20% in DMF (3 mL). After filtration, the resin is washed with DMF (3×4 mL) and DCM (3×4 mL). The resin is then treated with a soln. of Fmoc-NMeLeu-OH (253 mg, 0.675 mmol), TBTU (214 mg, 0.648 mmol), HOBt (103 mg, 0.675 mmol), and DIPEA (0.236 ml, 1.35 mmol) in DMF (3 mL) for 3 h. After filtration, the resin is washed with DMF (3×4 mL) and DCM (3×4 mL). The Fmoc deprotection is performed by treating the resin (2×5 min) with a soln. of piperidine/DBU/DMF (1/1/48) (2×3 mL×5 min). After filtration, the resin is washed with DMF (3×4 mL) and DCM (3×4 mL). The resin is then treated with a soln. of Pd(PPh₃)₄ (78 mg, 0.0675 mmol) and 1,3-dimethylbarbituric (106 mg, 0.675 mmol) in THF for 10 h. After filtration, the resin is washed successively with a soln. of 0.5% DIPEA in DMF (4×3 mL) and a soln. of 0.5% sodium diethyldithiocarbamate in DMF (4×3 mL) and finally with DMF (4×3 mL). The cyclisation of the linear peptide is performed by treating the resin with a soln. of pentafluorophenyl diphenylphosphinate (104 mg, 0.27 mmol) in DMF/DCM (2 mL) for 5 h. Finally, the macrocycle is cleaved from the resin using a soln. of TFA in DCM (1/1, 3 mL) for 2 h. After purification of the crude mix. by prep. HPLC (acidic), the title compound MC—COOH-8 (22 mg, 26%) is obtained as a white solid. LC-MS D t_{R}=1.03 min; [M+H]⁺=681.31.

Listed in Table MC—COOH—B below are carboxylic acids that are prepared from the corresponding starting materials in analogy to the solid phase synthesis described above for MC—COOH-8.

TABLE MC-COOH-B

| | | | | | t_{R} [min] | MS-data |
|---|---|---|---|---|---|---|
| | | SM | SM | | LC-MS | m/z |
| No. | Structure | BB-B | BB-D1 | Product | Method | [M + H]⁺ |
| MC- COOH- 9 | 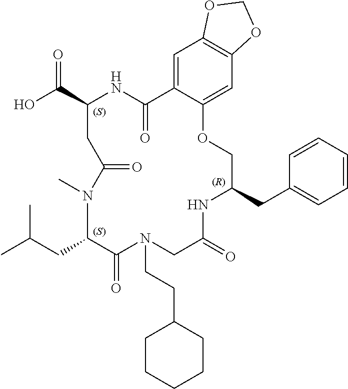 | B- Acid-4 | D1-2.2 | (7R,13S<17S)-7-Benzyl-11-(2- cyclohexylethyl)-13-isobutyl-14- methyl-9,12,15,19-tetraoxo- 6,7,8,9,10,11,12,13,14,15,16,17,18,19- tetradecahydro- [1,3]dioxolo[4',5':4,5]benzo[1,2- p][1]oxa[4,7,10,14] tetraazacycloheptadecine-17- carboxylic acid | 1.11 D | 707.31 |
| MC- COOH- 10 | 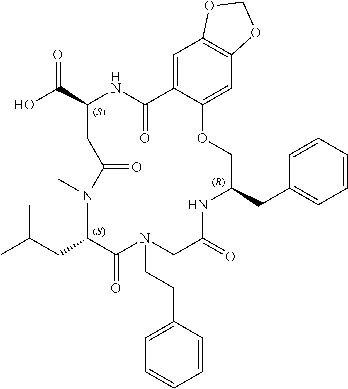 | B- Acid-4 | D1-2.4 | (7R,13S,17S)-7-Benzyl-13-isobutyl- 14-methyl-9,12,15,19-tetraoxo-11- phenethyl- 6,7,8,9,10,11,12,13,14,15,16,17,18,19- tetradecahydro- [1,3]dioxolo[4',5':4,5]benzo[1,2- p][1]oxa[4,7,10,14] tetraazacycloheptadecine-17- carboxylic acid | 0.98 D | 701.21 |
| MC- COOH- 11 | 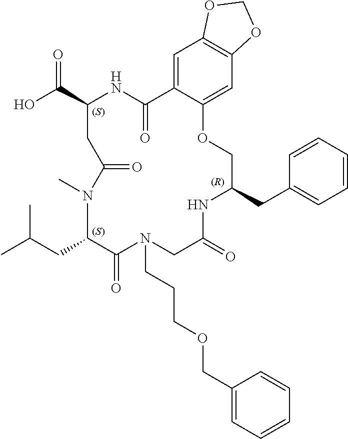 | B- Acid-4 | D1-2.5 | (7R,13S,17S)-7-Benzyl-11-(3- (benzyloxy)propyl)-13-isobutyl-14- methyl-9,12,15,19-tetraoxo- 6,7,8,9,10,11,12,13,14,15,16,17,18,19- tetradecahydro- [1,3]dioxolo[4',5':4,5]benzo[1,2- p][1]oxa[4,7,10,14] tetraazacycloheptadecine-17- carboxylic acid | 1.00 D | 745.31 |
| MC- COOH- 17 | 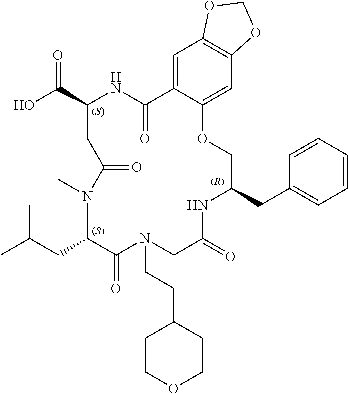 | B- Acid-4 | D1-2.6 | (7R,13S,17S)-7-benzyl-13-isobutyl- 14-methyl-9,12,15,19-tetraoxo-11-(2- (tetrahydro-2H-pyran-4-yl)ethyl)- 6,7,8,9,10,11,12,13,14,15,16,17,18,19- tetradecahydro- [1,3]dioxolo[4',5':4,5]benzo[1,2- p][1]oxa[4,7,10,14] tetraazacycloheptadecine-17- carboxylic acid | 0.84 D | 709.34 |

### (7R,13S,17S)-7-Benzyl-13-(cyclobutylmethyl)-11,14-dimethyl-9,12,15,19-tetraoxo-6,7,8,9,10,11,12,13,14,15,16,17,18,19-tetradecahydro-[1,3]dioxolo[4′,5′:4,5]benzo[1,2-p][1]oxa[4,7,10,14]tetraazacycloheptadecine-17-carboxylic acid (MC—COOH-12)

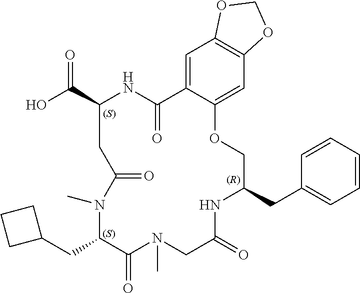

The title compound is prepared according to the solid phase-method described for MC—COOH-8, using B-Acid-4. Replacing D1-2.1 by commercially available N-(((9H-fluoren-9-yl)methoxy)carbonyl)-N-methylglycine and replacing Fmoc-NMeLeu-OH for D1-2.3. LC-MS B t_{R}=0.87 min; [M+H]⁺=623.25.

### (3R,9S,13S)-3-benzyl-7-(2-cyclohexylethyl)-9-(cyclopropylmethyl)-17,18-dimethoxy-10-methyl-5,8,11,15-tetraoxo-2,3,4,5,6,7,8,9,10,11,12,13,14,15-tetradecahydrobenzo[p][1]oxa[4,7,10,14]tetraazacycloheptadecine-13-carboxylic acid (MC—COOH-13)

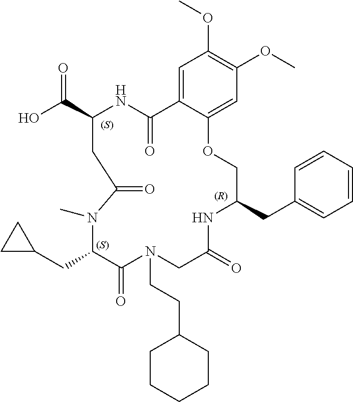

Step 1: HATU (7.45 g, 19 mmol) is added to a RT soln. of B-Acid-2 (7.46 g, 17.3 mmol), A-2.1 (5.70 g, 19 mmol) and DIPEA (11.9 mL, 69.7 mmol) in DMF (80 mL) and the RM is stirred at RT for 2 h. The solvent is evaporated and the crude product is purified by FC (eluting with DCM to DCM/MeOH 9:1) to give 4-allyl 1-benzyl (2-((R)-2-((tert-butoxycarbonyl)amino)-3-phenylpropoxy)-4,5-dimethoxybenzoyl)-L-aspartate HCl (11.71 g, 100%). LC-MS I: t_{R}=1.24 min; [M+H]⁺=677.42.

Step 2: 4 M HCl in dioxane (34.5 mL, 138 mmol) is added to 4-allyl 1-benzyl (2-((R)-2-amino-3-phenylpropoxy)-4,5-dimethoxybenzoyl)-L-aspartate HCl (11.712 g, 17.3 mmol) in DCM (135 mL). The mix. is stirred at RT for 4 h and evaporated to yield 4-allyl 1-benzyl (2-((R)-2-amino-3-phenylpropoxy)-4,5-dimethoxybenzoyl)-L-aspartate HCl (11.71 g, 100%) as a white solid which is used as such in the next step. LC-MS I: t_{R}=1.07 min; [M+H]⁺=577.36.

Step 3: IM-5.1 (639 mg, 2.44 mmol), 4-allyl 1-benzyl (2-((R)-2-amino-3-phenylpropoxy)-4,5-dimethoxybenzoyl)-L-aspartate HCl (1.695 g, 2.44 mmol), HATU (956 mg, 2.44 mmol) and DIPEA (2.44 mL, 14.3 mmol) are dissolved in DMF (12 mL). The mix. is stirred at RT for 2 h. The solvent is evaporated and the crude mix. is purified by FC (eluting with 0-5% MeOH in DCM) to give 4-allyl 1-benzyl (4,5-dimethoxy-2-((R)-2-(2-((2-nitrophenyl)sulfonamido)acetamido)-3-phenylpropoxy)benzoyl)-L-aspartate (964 mg, 48%) as a colourless oil. LC-MS B: t_{R}=1.1 min; [M+H]⁺=819.32.

Step 4: DIAD (227 mg, 0.968 mmol) is added to a RT soln. (degassed) of 4-allyl 1-benzyl (4,5-dimethoxy-2-((R)-2-(2-((2-nitrophenyl)sulfonamido)acetamido)-3-phenylpropoxy)benzoyl)-L-aspartate (864 mg, 0.88 mmol), 2-cyclohexylethanol (0.136 mL, 0.968 mmol), and PPh₃ (267 mg, 0.968 mmol) in DCM (5 mL). The RM is stirred at RT for 1 h, then the solvent is evaporated and the crude product purified by FC (eluting with 0% to 10% EtOAc in hept) to give 4-allyl 1-benzyl (2-((R)-2-(2-((N-(2-cyclohexylethyl)-2-nitrophenyl)sulfonamido)acetamido)-3-phenylpropoxy)-4,5-dimethoxybenzoyl)-L-aspartate (810 mg, 89%) as a white solid. LC-MS B: t_{R}=1.24 min; [M+H]⁺=929.38.

Step 5: K₂CO₃ (169 mg, 1.2 mmol) is added to a RT soln. of 4-allyl 1-benzyl (2-((R)-2-(2-((N-(2-cyclohexylethyl)-2-nitrophenyl)sulfonamido)acetamido)-3-phenylpropoxy)-4,5-dimethoxybenzoyl)-L-aspartate (811 mg, 0.8 mmol) and thiophenol (0.116 mL, 1.12 mmol) in DMF (4 mL). The RM is stirred at RT for 1 h, then the solvent is evaporated and the crude product purified by prep. HPLC (basic) to give 4-allyl 1-benzyl (2-((R)-2-(2-((2-cyclohexylethyl)amino)acetamido)-3-phenylpropoxy)-4,5-dimethoxybenzoyl)-L-aspartate (665 mg, 112%) as a colourless oil. LC-MS B: t_{R}=0.99 min; [M+H]⁺=744.41.

Step 6: HATU (42.2 mg, 0.108 mmol) is added to a RT soln. of 4-allyl 1-benzyl (2-((R)-2-(2-((2-cyclohexylethyl)amino)acetamido)-3-phenylpropoxy)-4,5-dimethoxybenzoyl)-L-aspartate (133 mg, 0.098 mmol), D2-1.4 (28.6 mg, 0.117 mmol), and DIPEA (0.0934 mL, 0.545 mmol) in DMF (2 mL). The RM is stirred at RT for 1 h, then the solvent is evaporated and the crude product purified by prep. HPLC (basic) to give 4-allyl 1-benzyl (2-(((6S,12R)-12-benzyl-8-(2-cyclohexylethyl)-6-(cyclopropylmethyl)-2,2,5-trimethyl-4,7,10-trioxo-3-oxa-5,8,11-triazatridecan-13-yl)oxy)-4,5-dimethoxybenzoyl)-L-aspartate (104 mg, 97.1%) as a white solid. LC-MS B: t_{R}=1.29 min; [M+H]⁺=969.66.

Step 7: 4 M HCl in dioxane (2.0 mL, 2.0 mmol) is added to 4-allyl 1-benzyl (2-(((6S,12R)-12-benzyl-8-(2-cyclohexylethyl)-6-(cyclopropylmethyl)-2,2,5-trimethyl-4,7,10-trioxo-3-oxa-5,8,11-triazatridecan-13-yl)oxy)-4,5-dimethoxybenzoyl)-L-aspartate (104 mg, 0.104 mmol) in DCM (3 mL). The RM is stirred at RT for 4 h, then the solvent is evaporated and the crude residue is dissolved in degassed DCM (3 mL) and treated with Pd(PPh₃)₄ (12.3 mg, 0.0104 mmol) and 1,3-dimethylbarbituric acid (32.9 mg, 0.208 mmol). The RM is stirred for 1 h at RT, then filtered through a Whatman filter and the solvent evaporated to give (S)-4-(benzyloxy)-3-(2-((R)-2-(2-((S)—N-(2-cyclohexylethyl)-3-cyclopropyl-2-(methylamino)propanamido)acetamido)-3-phenylpropoxy)-4,5-dimethoxybenzamido)-4-oxobutanoic acid (104 mg, 120%) as a colourless oil which is used as such in the next step. LC-MS B: t_{R}=0.94 min; [M+H]⁺=829.36.

Step 8: FDPP (38 mg, 0.14 mmol) is added to a RT soln. of (S)-4-(benzyloxy)-3-(2-((R)-2-(2-((S)—N-(2-cyclohexylethyl)-3-cyclopropyl-2-(methylamino)propanamido)acetamido)-3-phenylpropoxy)-4,5-dimethoxybenzamido)-4-oxobutanoic acid (40 mg, 0.05 mmol) in DCM. The RM is heated to 50° C. for 12 h, then the solvent is evaporated and the crude product purified by prep. HPLC (basic) to give benzyl (3R,9S,13S)-3-benzyl-7-(2-cyclohexylethyl)-9-(cyclopropylmethyl)-17,18-dimethoxy-10-methyl-5,8,11,15-tetraoxo-2,3,4,5,6,7,8,9,10,11,12,13,14,15 tetradecahydrobenzo[p][1]oxa[4,7,10,14]tetraazacycloheptadecine-13-carboxylate (11 mg, 13%) as a white solid. LC-MS B: t_{R}=1.18 min; [M+H]⁺=811.43.

Step 9: Pd/C (10%, 1.84 mg, 0.0017 mmol) is added to a RT soln. (degassed) of benzyl (3R,9S,13S)-3-benzyl-7-(2-cyclohexylethyl)-9-(cyclopropylmethyl)-17,18-dimethoxy-10-methyl-5,8,11,15-tetraoxo-2,3,4,5,6,7,8,9,10,11,12,13,14,15 tetradecahydrobenzo[p][1]oxa[4,7,10,14]tetraazacycloheptadecine-13-carboxylate (14 mg, 0.017 mmol) in MeOH (2 mL). The RM is stirred at RT under a H₂ atm for 1 h, then the mix. is filtered through a Whatman filter and the filtrate is concentrated to yield the title compound MC—COOH-13 (14 mg, 113%) as a colourless oil which is used as such in the next step. LC-MS B: t_{R}=1.01 min; [M+H]⁺=721.35.

### (3R,9S,13S)-3-Benzyl-7-(2-cyclohexylethyl)-9-(cyclopentylmethyl)-17,18-dimethoxy-10-methyl-5,8,11,15-tetraoxo-2,3,4,5,6,7,8,9,10,11,12,13,14,15-tetradecahydrobenzo[p][1]oxa[4,7,10,14]tetraazacycloheptadecine-13-carboxylic acid (MC—COOH-14)

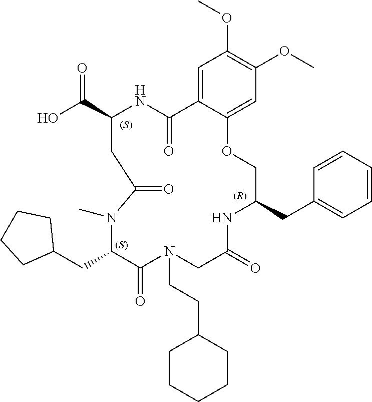

The title compound is prepared in analogy to the procedure described for MC—COOH-13, replacing in step 6, D2-1.4 with D2-1.1. LC-MS B: t_{R}=1.19 min; [M+H]⁺=896.38.

### (3R,9S,13S)-3-benzyl-9-isobutyl-17,18-dimethoxy-10-methyl-5,8,11,15-tetraoxo-7-(2-((RS)-tetrahydro-2H-pyran-2-yl)ethyl)-2,3,4,5,6,7,8,9,10,11,12,13,14,15-tetradecahydrobenzo[p][1]oxa[4,7,10,14]tetraazacycloheptadecine-13-carboxylic acid (MC—COOH-18)

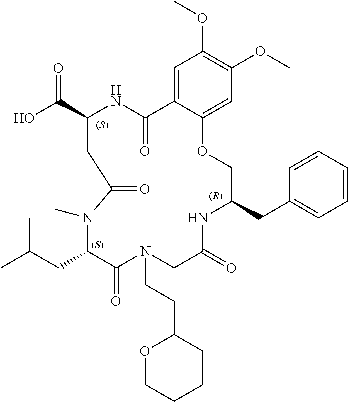

Steps 1-6: The intermediate is prepared in analogy to the procedure described for MC—COOH-13, following steps 1-6, where in step 4, 2-(tetrahydro-2H-pyran-2-yl)ethanol instead of 2-cyclohexylethanol, and in step 5, D2-3.1 instead of D2-1.4 are used.

Step 7: Pd(PPh₃)₄ (29.8 mg, 0.025 mmol) is added to a RT soln. (degassed) of the intermediate from step 6 (242 mg, 0.252 mmol) and 1,3-dimethylbarbituric acid (79.6 mg, 0.505 mmol) in DCM (2 mL) and the RM is stirred at RT for 1.5 h. The RM is filtered and concentrated and used as such in the next step. LC-MS I: t_{R}=0.68 min; [M+H]⁺=833.60.

Steps 8&9: The title compound MC—COOH-18 is prepared following the procedure described for MC—COOH-13, steps 8&9. LC-MS I: t_{R}=0.53/0.54 min; [M+H]⁺=725.50.

### (3R,9S,13S)-3-benzyl-9-isobutyl-17,18-dimethoxy-7-(3-methoxy-3-methylbutyl)-10-methyl-5,8,11,15-tetraoxo-2,3,4,5,6,7,8,9,10,11,12,13,14,15-tetradecahydrobenzo[p][1]oxa[4,7,10,14]tetraazacycloheptadecine-13-carboxylic acid (MC—COOH-19)

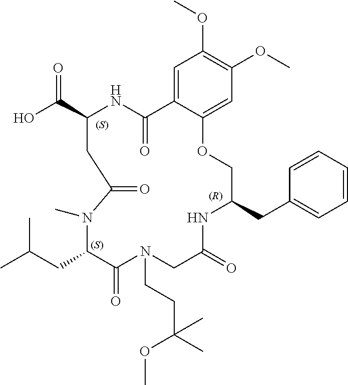

The title compound is prepared in analogy to the 9-step synthesis described for MC—COOH-18. In step 4, 3-methoxy-3-methyl-1-butanol instead of 2-(tetrahydro-2H-pyran-2-yl)ethanol is used. LC-MS B: t_{R}=0.90 min; [M+H]⁺=713.36.

### (3S,7S,13R)-13-benzyl-7-isobutyl-6-methyl-1,5,8,11-tetraoxo-9-(((R)-tetrahydro-2H-pyran-2-yl)methyl)-1,2,3,4,5,6,7,8,9,10,11,12,13,14-tetradecahydronaphtho[1,2-p][1]oxa[4,7,10,14]tetraazacycloheptadecine-3-carboxylic acid (MC—COOH-20)

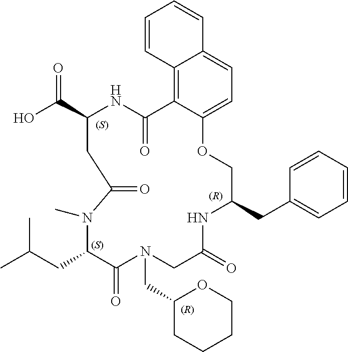

Steps 1&2: 4-Allyl 1-benzyl (2-((R)-2-amino-3-phenylpropoxy)-1-naphthoyl)-L-aspartate hydrochloride is prepared following the synthesis of MC—COOH-13, steps 1&2, using A-2.1 and B-Acid-5. LC-MS B: t_{R}=0.9 min; [M+H]⁺=567.28.

Step 3: HATU (87.5 mg, 0.23 mmol) is added to a RT soln. of 4-allyl 1-benzyl (2-((R)-2-amino-3-phenylpropoxy)-1-naphthoyl)-L-aspartate hydrochloride (127 mg, 0.20 mmol), C-16.4 (84.6 mg, 0.22 mmol) and DIPEA (0.14 mL, 0.8 mmol) in DMF (3 mL) and the RM is stirred at RT for 2 h. The solvent is evaporated and the crude product is purified by prep. HPLC (basic) to yield 4-allyl 1-benzyl (2-(((2R,8S)-2-benzyl-8-isobutyl-9-methyl-4,7,10-trioxo-6-(((R)-tetrahydro-2H-pyran-2-yl)methyl)-11-oxa-3,6,9-triazatetradec-13-en-1-yl)oxy)-1-naphthoyl)-L-aspartate (142 mg, 76%). LC-MS I: t_{R}=1.34 min; [M+H]⁺=933.56.

Step 4: Pd(PPh₃)₄ (29.8 mg, 0.025 mmol) is added to a RT soln. (degassed) of 4-allyl 1-benzyl (2-(((2R,8S)-2-benzyl-8-isobutyl-9-methyl-4,7,10-trioxo-6-(((R)-tetrahydro-2H-pyran-2-yl)methyl)-11-oxa-3,6,9-triazatetradec-13-en-1-yl)oxy)-1-naphthoyl)-L-aspartate (145 mg, 0.152 mmol) and 1,3-dimethylbarbituric acid (47.8 mg, 0.30 mmol) in DCM (3 mL) and the RM is stirred at RT for 1 h. The RM is filtered and concentrated and used as such in the next step. LC-MS I: t_{R}=0.68 min; [M+H]⁺=809.52.

Steps 5&6: The title compound MC—COOH-20 is prepared following steps 8&9 described for MC—COOH-13. LC-MS D: t_{R}=0.98 min; [M+H]⁺=701.10.

Listed in Table MC—COOH—C below are carboxylic acids that are prepared from the corresponding starting materials in analogy to the synthesis described above for MC—COOH-20.

TABLE MC-COOH-C

| | | | | | t_{R} [min] | MS-data |
|---|---|---|---|---|---|---|
| | | SM | SM | | LC-MS | m/z |
| No. | Structure | BB-B | BB-C | Product | Method | [M + H]⁺ |
| MC- COOH- 21 | 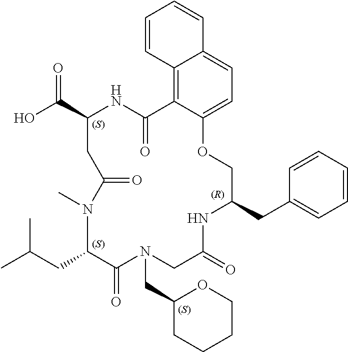 | B- Acid- 5 | C-16.5 | (3S,7S,13R)-13-benzyl-7-isobutyl-6- methyl-1,5,8,11-tetraoxo-9-(((S)- tetrahydro-2H-pyran-2-yl)methyl)- 1,2,3,4,5,6,7,8,9,10,11,12,13,14- tetradecahydronaphtho[1,2- p][1]oxa[4,7,10,14] tetraazacycloheptadecine-3-carboxylic acid | 1.00 D | 709.01 |
| MC- COOH- 22 | 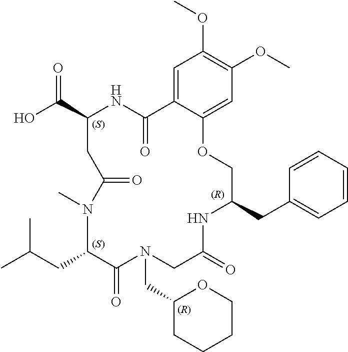 | B- Acid- 2 | C-16.4 | (3R,9S,13S)-3-benzyl-9-isobutyl- 17,18-dimethoxy-10-methyl- 5,8,11,15-tetraoxo-7-(((R)-tetrahydro- 2H-pyran-2-yl)methyl)- 2,3,4,5,6,7,8,9,10,11,12,13,14,15- tetradecahydrobenzo[p][1]oxa [4,7,10,14] tetraazacycloheptadecine- 13-carboxylic acid | 0.92 B | 711.34 |
| MC- COOH- 23 | 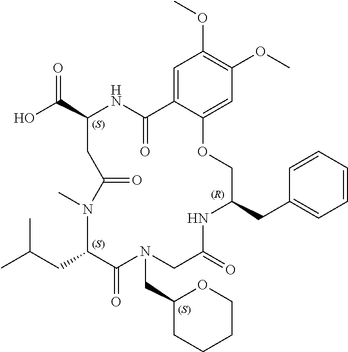 | B- Acid- 2 | C-16.5 | (3R,9S,13S)-3-benzyl-9-isobutyl- 17,18-dimethoxy-10-methyl- 5,8,11,15-tetraoxo-7-(((S)-tetrahydro- 2H-pyran-2-yl)methyl)- 2,3,4,5,6,7,8,9,10,11,12,13,14,15- tetradecahydrobenzo[p][1]oxa [4,7,10,14] tetraazacycloheptadecine- 13-carboxylic acid | 0.94 | 711.34 |

### Synthesis of Compounds of Formula (I)

### General Method: GM-1

A stock soln. of the corresponding MC—COOH (0.05 mmol) and DIPEA (0.15 mmol) in DMF (0.5 mL) is added to each tube containing the corresponding amine AM. After stirring for 2 min another stock soln. of HATU (0.05 mmol) in DMF (0.5 mL) is added to each tube and the resulting mixtures are shaken at RT for 1 h. The mixtures are directly purified by prep. HPLC (basic) and the fractions are dried in a Genevac HT12 vacuum centrifuge at 40° C. to obtain the expected products. In some cases, chiral chromatography is used to obtain the desired product as a pure stereoisomer.

### General Method: GM-2

### Example 222: (4aR,7R,18S,22S)-7-Benzyl-22-isobutyl-N-(3-methoxyphenethyl)-21-methyl-5,16,20,23-tetraoxo-1,2,4,4a,5,6,7,8,16,17,18,19,20,21,22,23-hexadecahydronaphtho[1,2-p][1,4]oxazino[3,4-f][1]oxa[4,7,10,14]tetraazacycloheptadecine-18-carboxamide

Step 1: HATU (201 mg, 0.53 mmol) is added to a RT soln. of B-1.13 (239 mg, 0.53 mmol), C-2.1 (203 mg, 0.53 mmol) and DIPEA (0.27 mL, 1.58 mmol) in DMF (5 mL) and the RM is stirred for 30 min. The RM is then directly purified by prep. HPLC (basic) to give benzyl 2-((R)-2-((R)-4-(N-(tert-butoxycarbonyl)-N-methyl-L-leucyl)morpholine-3-carboxamido)-3-phenylpropoxy)-1-naphthoate as a white solid. LC-MS I: t_{R}=1.40 min; [M+H]⁺=752.46.

Step 2: A soln. of benzyl 2-((R)-2-((R)-4-(N-(tert-butoxycarbonyl)-N-methyl-L-leucyl)morpholine-3-carboxamido)-3-phenylpropoxy)-1-naphthoate (308 mg, 0.41 mmol) in EtOH (3 mL) is evacuated/purged with N₂ (3×) before 10% Pd/C (22 mg, 5 mol %) is added. The RM is evacuated/purged with H₂ (3×) and stirred under a H₂ atm for 2 h. The RM is filtered through a pad of celite and the filtrate concentrated in vacuo to give 2-((R)-2-((R)-4-(N-(tert-butoxycarbonyl)-N-methyl-L-leucyl)morpholine-3-carboxamido)-3-phenylpropoxy)-1-naphthoic acid as a white solid. LC-MS I: t_{R}=0.65 min; [M+H]⁺=662.38.

Step 3: HATU (96 mg, 0.24 mmol) is added to a RT soln. of 2-((R)-2-((R)-4-(N-(tert-butoxycarbonyl)-N-methyl-L-leucyl)morpholine-3-carboxamido)-3-phenylpropoxy)-1-naphthoic acid (160 mg, 0.24 mmol), A-1.1 (78 mg, 0.24 mmol) and DIPEA, (82 μL, 0.48 mmol) in DMF (3 mL) and the RM is stirred for 1h. The RM is then directly purified by prep. HPLC (basic) to give tert-butyl (S)-3-(2-((R)-2-((R)-4-(N-(tert-butoxycarbonyl)-N-methyl-L-leucyl)morpholine-3-carboxamido)-3-phenylpropoxy)-1-naphthamido)-4-((3-methoxyphenethyl)amino)-4-oxobutanoate as a white solid. LC-MS I: t_{R}=1.36 min; [M+H]⁺=966.74.

Step 4: TFA (2.1 mL, 28 mmol) is added to a RT soln. of tert-butyl (S)-3-(2-((R)-2-((R)-4-(N-(tert-butoxycarbonyl)-N-methyl-L-leucyl)morpholine-3-carboxamido)-3-phenylpropoxy)-1-naphthamido)-4-((3-methoxyphenethyl)amino)-4-oxobutanoate (174 mg, 0.18 mmol) in DCM (5 mL) and the RM is stirred for 2 h. The RM is concentrated in vacuo and the residue is re-dissolved in DCM and again concentrated in vacuo (2×). The residue is dissolved in DMF (4 mL) before DIPEA (0.24 mL, 1.4 mmol) and HATU (80 mg, 0.21 mmol) are added and the RM is stirred for 1 h. The RM is then directly purified by prep. HPLC (basic) to give the title compound as a white solid. LC-MS I: t_{R}=1.09 min; [M+H]⁺=792.45.

Note: In cases where the product of step 1 above is a methyl or ethyl ester instead of the described benzyl ester (e.g. Example 228 below), a basic hydrolysis using 10 eq. 2M aq. NaOH soln. in MeOH at RT or heated up to 80° C. is performed. The subsequent reaction sequence then remains the same as described for Example 222. In some cases, chiral chromatography is used to obtain the desired product as a pure stereoisomer.

### General Method: GM-3

### Example 689: (3'S,7'S,13′R)-13′-Benzyl-N-(2-(3-cyclopropylisoxazol-5-yl)ethyl)-20′-fluoro-7′-isobutyl-6′,9′-dimethyl-1′,5′,8′,11′-tetraoxo-2′,3′,4′,5′,6′,7′,8′,9′,11′,12′,13′,14′-dodecahydro-1′H-spiro[cyclobutane-1,10′-[1]oxa[4,7,10,14]tetraazacycloheptadecino[17,16-c]quinoline]-3′-carboxamide

Step 1: HATU (169 mg, 0.435 mmol) is added to a RT soln. of B-1.19 (200 mg, 0.396 mmol), D1-3.0 (95 mg, 0.415 mmol), and DIPEA (0.34 mL, 1.98 mmol) in DMF (4 mL) and the RM is stirred for 16 h. The RM is purified directly by prep. HPLC (basic) to yield 3-((R)-2-{[1-(tert-butoxycarbonyl-methyl-amino)-cyclobutanecarbonyl]-amino}-3-phenyl-propoxy)-6-fluoro-quinoline-4-carboxylic acid benzyl ester (220 mg, 87%) which is used as such in the next step.

Step 2: 4.0 M HCl in dioxane (0.385 mL, 1.54 mmol) is added to a RT soln. of 3-((R)-2-{[1-(tert-butoxycarbonyl-methyl-amino)-cyclobutanecarbonyl]-amino}-3-phenyl-propoxy)-6-fluoro-quinoline-4-carboxylic acid benzyl ester (220 mg, 0.385 mmol) in DCM (2 mL) and the RM is stirred at RT for 2 h. The solvent is evaporated and crude benzyl (R)-6-fluoro-3-(2-(1-(methylamino)cyclobutane-1-carboxamido)-3-phenylpropoxy)quinoline-4-carboxylate (195 mg) is used as such in the next step.

Step 3: HATU (164 mg, 0.423 mmol) is added to a RT soln. of benzyl (R)-6-fluoro-3-(2-(1-(methylamino)cyclobutane-1-carboxamido)-3-phenylpropoxy)quinoline-4-carboxylate (195 mg, 0.385 mmol), Boc-N-methyl-L-leucine (99.4 mg, 0.404 mmol), and DIPEA (0.33 mL, 1.93 mmol) in DMF (4 mL) and the RM is stirred at RT for 16 h. The RM is purified directly by prep. HPLC (basic) to yield benzyl 3-((R)-2-(1-((S)-2-((tert-butoxycarbonyl)(methyl)amino)-N,4-dimethylpentanamido)cyclobutane-1-carboxamido)-3-phenylpropoxy)-6-fluoroquinoline-4-carboxylate (207 mg, 70%). LC-MS I: t_{R}=1.45-1.48 min; [M+H]⁺=769.48.

Step 4: LiOH·H₂O (35.1 mg, 0.84 mmol) is added to a RT soln. of benzyl 3-((R)-2-(1-((S)-2-((tert-butoxycarbonyl)(methyl)amino)-N,4-dimethylpentanamido)cyclobutane-1-carboxamido)-3-phenylpropoxy)-6-fluoroquinoline-4-carboxylate (207 mg, 0.270 mmol) in THF/H₂O 2/1 (3 mL) and the RM is stirred at 60° C. for 3 d. The THF is evaporated and the residue is acidified with 1 M HCl to pH 1 and extracted with EtOAc (3×). The combined org. layers are dried (MgSO₄), filtered, and evaporated to yield crude 3-((R)-2-(1-((S)-2-((tert-butoxycarbonyl)(methyl)amino)-N,4-dimethylpentanamido)cyclobutane-1-carboxamido)-3-phenylpropoxy)-6-fluoroquinoline-4-carboxylic acid (182 mg) which is used as such in the next step. LC-MS I: t_{R}=0.70 min; [M+H]⁺=679.45.

Step 5: HATU (28.6 mg, 0.0737 mmol) is added to a RT soln. of 3-((R)-2-(1-((S)-2-((tert-butoxycarbonyl)(methyl)amino)-N,4-dimethylpentanamido)cyclobutane-1-carboxamido)-3-phenylpropoxy)-6-fluoroquinoline-4-carboxylic acid (45.5 mg, 0.067 mmol), A-1.24 (22 mg, 0.07 mmol), and DIPEA (0.0573 mL, 0.335 mmol) in DMF (2 mL) and the RM is stirred at RT for 16 h. The RM is purified directly by prep. HPLC (basic) to yield tert-butyl (S)-3-(3-((R)-2-(1-((S)-2-((tert-butoxycarbonyl)(methyl)amino)-N,4-dimethylpentanamido)cyclobutane-1-carboxamido)-3-phenylpropoxy)-6-fluoroquinoline-4-carboxamido)-4-((2-(3-cyclopropylisoxazol-5-yl)ethyl)amino)-4-oxobutanoate (44 ma. 67%). LC-MS B: t_{R}=1.24 min: [M+H]⁺=984.40.

Step 6: TFA (2 mL, 26 mmol) is added to a RT soln. of tert-butyl (S)-3-(3-((R)-2-(1-((S)-2-((tert-butoxycarbonyl)(methyl)amino)-N,4-dimethylpentanamido)cyclobutane-1-carboxamido)-3-phenylpropoxy)-6-fluoroquinoline-4-carboxamido)-4-((2-(3-cyclopropylisoxazol-5-yl)ethyl)amino)-4-oxobutanoate (44 mg, 0.045 mmol) in DMF (2 mL) and the resulting mix. is stirred at RT for 2 h. The mix. is concentrated and co-evaporated with DCM (2×) to yield (S)-4-((2-(3-cyclopropylisoxazol-5-yl)ethyl)amino)-3-(3-((R)-2-(1-((S)—N,4-dimethyl-2-(methylamino)pentanamido)cyclobutane-1-carboxamido)-3-phenylpropoxy)-6-fluoroquinoline-4-carboxamido)-4-oxobutanoic acid trifluoroacetate (53 mg) which is used as such in the next step. LC-MS I: t_{R}=0.58 min; [M+H]⁺=828.28.

Step 7: HATU (108 mg, 0.283 mmol) is added to a RT soln. of (S)-4-((2-(3-cyclopropylisoxazol-5-yl)ethyl)amino)-3-(3-((R)-2-(1-((S)—N,4-dimethyl-2-(methylamino)pentanamido)cyclobutane-1-carboxamido)-3-phenylpropoxy)-6-fluoroquinoline-4-carboxamido)-4-oxobutanoic acid trifluoroacetate (53 mg) and DIPEA (0.162 mL, 0.945 mmol) in DMF (0.5 mL)/DCM (4.5 mL), and the RM is stirred at RT for 16 h. The RM is concentrated and purified by prep. HPLC (basic) to yield the title compound (30 mg) as a solid. LC-MS I: t_{R}=1.07 min; [M+H]⁺=810.29.

### General Method: GM-4

### Example 700: (3S,7S,13R)—N-(2-(Benzo[d][1,3]dioxol-5-yl)ethyl)-13-benzyl-9-(((S)-chroman-3-yl)methyl)-7-isobutyl-6-methyl-1,5,8,11-tetraoxo-1,2,3,4,5,6,7,8,9,10,11,12,13,14-tetradecahydronaphtho[1,2-p][1]oxa[4,7,10,14]tetraazacycloheptadecine-3-carboxamide

Steps 1&2: Allyl (S)-3-(2-((R)-2-amino-3-phenylpropoxy)-1-naphthamido)-4-((2-(benzo[d][1,3]dioxol-5-yl)ethyl)amino)-4-oxobutanoate hydrochloride is prepared following the synthesis described for GM-5, steps 1&2, using A-2.2 and B-Acid-5. LC-MS B: t_{R}=0.88 min; [M+H]⁺=624.23.

Step 3: HATU (41.8 mg, 0.11 mmol) is added to a RT soln. of allyl (S)-3-(2-((R)-2-amino-3-phenylpropoxy)-1-naphthamido)-4-((2-(benzo[d][1,3]dioxol-5-yl)ethyl)amino)-4-oxobutanoate hydrochloride (66 mg, 0.1 mmol), C-16.2 (47.6 mg, 0.11 mmol), and DIPEA (0.051 mL, 0.3 mmol) in DMF (1 mL) and the RM is stirred at RT for 30 min.

Purification by prep. HPLC (acidic) yields allyl (S)-4-((2-(benzo[d][1,3]dioxol-5-yl)ethyl)amino)-3-(2-(((2R,8S)-2-benzyl-6-(((S)-chroman-3-yl)methyl)-8-isobutyl-9-methyl-4,7,10-trioxo-11-oxa-3,6,9-triazatetradec-13-en-1-yl)oxy)-1-naphthamido)-4-oxobutanoate (87 mg, 84%) as a white solid. LC-MS I: t_{R}=1.33 min; [M+H]⁺=1038.74.

Step 4: Pd(Ph₃)₄ (9.63 mg, 0.0082 mmol) is added to a RT soln. of allyl (S)-4-((2-(benzo[d][1,3]dioxol-5-yl)ethyl)amino)-3-(2-(((2R,8S)-2-benzyl-6-(((S)-chroman-3-yl)methyl)-8-isobutyl-9-methyl-4,7,10-trioxo-11-oxa-3,6,9-triazatetradec-13-en-1-yl)oxy)-1-naphthamido)-4-oxobutanoate (87.7 mg, 0.082 mmol) and 1,3-dimethylbarbituric acid (25.8 mg, 0.163 mmol) in DCM (1 mL) and the RM is stirred at RT for 2 h. The RM is concentrated to yield (S)-4-((2-(benzo[d][1,3]dioxol-5-yl)ethyl)amino)-3-(2-((R)-2-(2-((S)—N—(((S)-chroman-3-yl)methyl)-4-methyl-2-(methylamino)pentanamido)acetamido)-3-phenylpropoxy)-1-naphthamido)-4-oxobutanoic acid which is used as such in the next step. LC-MS I: t_{R}=0.69 min; [M+H]⁺=914.75.

Step 5: HATU (31.1 mg, 0.082 mmol) is added to a RT soln. of (S)-4-((2-(benzo[d][1,3]dioxol-5-yl)ethyl)amino)-3-(2-((R)-2-(2-((S)—N—(((S)-chroman-3-yl)methyl)-4-methyl-2-(methylamino)-pentanamido)acetamido)-3-phenylpropoxy)-1-naphthamido)-4-oxobutanoic acid (74.7 mg, 0.082 mmol) and DIPEA (70 μL, 0.41 mmol) in DMF (1 mL) and the RM is stirred at RT for 30 min. Purification by prep. HPLC (basic) yields the title compound (49.6 mg, 97%) as a white solid. LC-MS I: t_{R}=1.19 min; [M+H]⁺=896.69.

### Example 976: (3S,7S,10R,13R)-13-Benzyl-N-(2-(3-cyclopropyl-1,2,4-oxadiazol-5-yl)ethyl)-10-(2-ethoxyethyl)-20-fluoro-7-isobutyl-6,9-dimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide

Step 1: HATU (424 mg, 1.11 mmol) is added to a RT solution of B-Acid-8 (394 mg, 0.928 mmol), A-1.22 (361 mg, 1.11 mmol) and DIPEA (0.477 mL, 2.78 mmol) in DMF (5 mL) under argon and the resulting mixture is stirred for 1h. The RM is partioned between water and EtOAc. The layers are separated and the inorg. layer is extracted further with EtOAc (2×). The combined org. extracts are washed with water and brine, dried over Na₂SO₄, filtered and evaporated under reduced pressure to give the crude product; It is purified by FC, eluting with 0% to 100% EtOAc in Heptane to give tert-butyl (S)-3-(6-((R)-2-(((allyloxy)carbonyl)amino)-3-phenylpropoxy)-3-fluoroquinoline-5-carboxamido)-4-((2-(3-cyclopropyl-1,2,4-oxadiazol-5-yl)ethyl)amino)-4-oxobutanoate as a beige solid (334 mg). LC-MS B: t_{R}=1.09 min; [M+H]⁺=731.48.

Step 2: Under an argon atmosphere, tert-butyl (S)-3-(6-((R)-2-(((allyloxy)carbonyl)amino)-3-phenylpropoxy)-3-fluoroquinoline-5-carboxamido)-4-((2-(3-cyclopropyl-1,2,4-oxadiazol-5-yl)ethyl)amino)-4-oxobutanoate (334 mg, 0.457 mmol) is dissolved in MeOH (10 mL) and 1,3-dimethylbarbituric acid (144 mg, 0.914 mmol) then Pd(PPh₃)₄ (27.2 mg, 0.0229 mmol) are added to the solution. The RM is stirred at RT for 1 hr to reach full conversion. The RM is evaporated under reduced pressure and the resulting crude is purified by FC eluting from 0% to 20% MeOH in DCM to yield tert-butyl (S)-3-(6-((R)-2-amino-3-phenylpropoxy)-3-fluoroquinoline-5-carboxamido)-4-((2-(3-cyclopropyl-1,2,4-oxadiazol-5-yl)ethyl)amino)-4-oxobutanoate as a brown oil (142 mg). LC-MS B: t_{R}=0.81 min; [M+H]⁺=647.46.

Step 3: HATU (100 mg, 0.264 mmol) is added to a RT solution of C-18.9 (85.5 mg, 0.22 mmol), tert-butyl (S)-3-(6-((R)-2-amino-3-phenylpropoxy)-3-fluoroquinoline-5-carboxamido)-4-((2-(3-cyclopropyl-1,2,4-oxadiazol-5-yl)ethyl)amino)-4-oxobutanoate (142 mg, 0.22 mmol) and DIPEA (0.113 mL, 0.66 mmol) in DMF (2 mL) under argon and the RM is stirred for 1h. The mixture is partioned between water and EtOAc. The layers are separated and the inorg. layer is extracted further with EtOAc (2×). The combined org. extracts are washed with water and brine, dried over Na₂SO₄, filtered and evaporated under reduced pressure to give the crude product. Purification by FC, eluting with 50% EtOAc in Heptane gives tert-butyl (S)-3-(6-(((6S,9RS,12R)-12-benzyl-9-(2-ethoxyethyl)-6-isobutyl-2,2,5,8-tetramethyl-4,7,10-trioxo-3-oxa-5,8,11-triazatridecan-13-yl)oxy)-3-fluoroquinoline-5-carboxamido)-4-((2-(3-cyclopropyl-1,2,4-oxadiazol-5-yl)ethyl)amino)-4-oxobutanoate as a white powder (114 mg). LC-MS B: t_{R}=1.25 min; [M+H]⁺=1017.93.

Step 4: The title compound is prepared as a white powder from tert-butyl (S)-3-(6-(((6S,9RS,12R)-12-benzyl-9-(2-ethoxyethyl)-6-isobutyl-2,2,5,8-tetramethyl-4,7,10-trioxo-3-oxa-5,8,11-triazatridecan-13-yl)oxy)-3-fluoroquinoline-5-carboxamido)-4-((2-(3-cyclopropyl-1,2,4-oxadiazol-5-yl)ethyl)amino)-4-oxobutanoate following the conditions described for GM-2, Step 4. LC-MS I: t_{R}=1.06 min; [M+H]⁺=843.73.

### General Method: GM-5

### Example 713: (3S,7S,10R,13R)-13-Benzyl-10-((benzyloxy)methyl)-7-isobutyl-N-(3-methoxyphenethyl)-6,9-dimethyl-1,5,8,11-tetraoxo-1,2,3,4,5,6,7,8,9,10,11,12,13,14-tetradecahydronaphtho[1,2-p][1]oxa[4,7,10,14]tetraazacycloheptadecine-3-carboxamide

Step 1: HATU (1.15 g, 2.95 mmol) is added to a RT soln. of A-2.3 (1.0 g, 2.95 mmol) B-Acid-5 (1.26 g, 2.95 mmol) and DIPEA (2.1 mL, 11.9 mmol) in DCM (10 mL) and the RM is stirred at RT overnight. Sat. aq. NaHCO₃ is added and the mix. is extracted with DCM. The org. layer is concentrated and the crude product purified by FC (eluting with 0% to 50% EtOAc in hept) to yield allyl (S)-3-(2-((R)-2-((tert-butoxycarbonyl)amino)-3-phenylpropoxy)-1-naphthamido)-4-((3-methoxyphenethyl)amino)-4-oxobutanoate (1.65 g, 79%). LC-MS B: t_{R}=1.16 min; [M+H]⁺=710.37.

Step 2: 4 M HCl in dioxane (2.33 mL, 9.31 mmol) is added to allyl (S)-3-(2-((R)-2-((tert-butoxycarbonyl)amino)-3-phenylpropoxy)-1-naphthamido)-4-((3-methoxyphenethyl)amino)-4-oxobutanoate (1.65 g, 2.33 mmol) in DCM (10 mL) and the RM is stirred at RT for 2 h. The mix. is concentrated to yield allyl (S)-3-(2-((R)-2-amino-3-phenylpropoxy)-1-naphthamido)-4-((3-methoxyphenethyl)amino)-4-oxobutanoate (1.7 g, 120%) which is used as such in the next step. LC-MS B: t_{R}=1.08 min; [M+H]⁺=610.43.

Step 3: HATU (42.7 mg, 0.11 mmol) is added to a RT soln. of allyl (S)-3-(2-((R)-2-amino-3-phenylpropoxy)-1-naphthamido)-4-((3-methoxyphenethyl)amino)-4-oxobutanoate (64.6 mg, 0.1 mmol), D1-1.1 (34 mg, 0.11 mmol) and DIPEA (0.051 mL, 0.3 mmol) in DMF (1 mL) and the RM is stirred at RT for 30 min. Purification by prep. HPLC (acidic) yields allyl (S)-3-(2-((R)-2-((R)-3-(benzyloxy)-2-((tert-butoxycarbonyl)(methyl)amino)propanamido)-3-phenylpropoxy)-1-naphthamido)-4-((3-methoxyphenethyl)amino)-4-oxobutanoate (47 mg, 52%) as the major epimer. LC-MS B: t_{R}=1.22 min; [M+H]⁺=901.13. The minor epimer allyl (S)-3-(2-((R)-2-((S)-3-(benzyloxy)-2-((tert-butoxycarbonyl)(methyl)amino)propanamido)-3-phenylpropoxy)-1-naphthamido)-4-((3-methoxyphenethyl)amino)-4-oxobutanoate (21 mg, 24%) is also isolated. LC-MS B: t_{R}=1.24 min; [M+H]⁺=901.14.

Step 4: 4 M HCl in dioxane (0.1 mL, 0.4 mmol) is added to a RT soln. of allyl (S)-3-(2-((R)-2-((R)-3-(benzyloxy)-2-((tert-butoxycarbonyl)(methyl)amino)propanamido)-3-phenylpropoxy)-1-naphthamido)-4-((3-methoxyphenethyl)amino)-4-oxobutanoate (47 mg, 0.051 mmol) in DCM (1 mL) and the RM is stirred at RT for 1 h. The RM is concentrated to yield allyl (S)-3-(2-((R)-2-((R)-3-(benzyloxy)-2-(methylamino)propanamido)-3-phenylpropoxy)-1-naphthamido)-4-((3-methoxyphenethyl)amino)-4-oxobutanoate hydrochloride (42.8 mg, 100%) as a white solid which is used as such in the next step. LC-MS B: t_{R}=0.97 min; [M+H]⁺=801.05.

Step 5: HATU (21.8 mg, 0.056 mmol) is added to a RT soln. of allyl (S)-3-(2-((R)-2-((R)-3-(benzyloxy)-2-(methylamino)propanamido)-3-phenylpropoxy)-1-naphthamido)-4-((3-methoxyphenethyl)amino)-4-oxobutanoate hydrochloride (42.8 mg, 0.0511 mmol), D2-3.1 (12.9 mg, 0.056 mmol) and DIPEA (0.026 mL, 0.153 mmol) in DMF (0.5 mL) and the RM is stirred at RT for 1 h. Purification by prep. HPLC (acidic) yields allyl (S)-3-(2-(((2R,5R,8S)-2-benzyl-5-((benzyloxy)methyl)-8-isobutyl-6,9-dimethyl-4,7,10-trioxo-11-oxa-3,6,9-triazatetradec-13-en-1-yl)oxy)-1-naphthamido)-4-((3-methoxyphenethyl)amino)-4-oxobutanoate (41.5 mg, 80%) as a white solid. LC-MS B: t_{R}=1.26 min; [M+H]⁺=1012.27.

Step 6: Pd(Ph₃)₄ (4.83 mg, 0.004 mmol) is added to a RT soln. of allyl (S)-3-(2-(((2R,5R,8S)-2-benzyl-5-((benzyloxy)methyl)-8-isobutyl-6,9-dimethyl-4,7,10-trioxo-11-oxa-3,6,9-triazatetradec-13-en-1-yl)oxy)-1-naphthamido)-4-((3-methoxyphenethyl)amino)-4-oxobutanoate (41.5 mg, 0.04 mmol) and 1,3-dimethylbarbituric acid (12.9 mg, 0.08 mmol) in DCM (1 mL) and the RM is stirred at RT for 1 h. The RM is concentrated to yield (S)-3-(2-((R)-2-((R)-3-(benzyloxy)-2-((S)—N,4-dimethyl-2-(methylamino)pentanamido)propanamido)-3-phenylpropoxy)-1-naphthamido)-4-((3-methoxyphenethyl)amino)-4-oxobutanoic acid which is used as such in the next step. LC-MS B: t_{R}=0.94 min; [M+H]⁺=888.15.

Step 7: HATU (15.9 mg, 0.041 mmol) is added to a RT soln. of (S)-3-(2-((R)-2-((R)-3-(benzyloxy)-2-((S)—N,4-dimethyl-2-(methylamino)pentanamido)propanamido)-3-phenylpropoxy)-1-naphthamido)-4-((3-methoxyphenethyl)amino)-4-oxobutanoic acid (36.4 mg, 0.041 mmol) and DIPEA (35.1 μL, 0.205 mmol) in DMF (1 mL) and the RM is stirred at RT for 20 min. Purification by prep. HPLC (acidic) yields the title compound (29.3 mg, 82%) as pale yellow solid. LC-MS B: t_{R}=1.18 min; [M+H]⁺=870.1.

### General Method: GM-6

### Example 722: (3S,7S,10R,13R)-10,13-Dibenzyl-20-fluoro-7-isobutyl-6,9-dimethyl-N-(2-(3-methylisoxazol-5-yl)ethyl)-1,5,8,11-tetraoxo-1,2,3,4,5,6,7,8,9,10,11,12,13,14-tetradecahydro-[1]oxa[4,7,10,14]tetraazacycloheptadecino[17,16-c]quinoline-3-carboxamide

Step 1: HATU (192 mg, 0.49 mmol) is added to a RT soln. of B-1.19 (227 mg, 0.45 mmol), C-17.1 (178 mg, 0.45 mmol), and DIPEA (0.39 mL, 0.3 mmol) in DMF (4 mL) and the RM is stirred at RT for 16 h. Purification by prep. HPLC (basic) yields benzyl 3-(((2R,5R,8S)-2,5-dibenzyl-8-isobutyl-6,9-dimethyl-4,7,10-trioxo-11-oxa-3,6,9-triazatetradec-13-en-1-yl)oxy)-6-fluoroquinoline-4-carboxylate (269 mg, 70%). LC-MS I: t_{R}=1.42 min; [M+H]⁺=803.46.

Step 2: LiOH·H₂O (35.1 mg, 0.84 mmol) is added to a RT soln. of benzyl 3-(((2R,5R,8S)-2,5-dibenzyl-8-isobutyl-6,9-dimethyl-4,7,10-trioxo-11-oxa-3,6,9-triazatetradec-13-en-1-yl)oxy)-6-fluoroquinoline-4-carboxylate (269 mg) in THF/H₂O 2/1 (3 mL) and the RM is stirred at 60° C. for 3 d. The THF is evaporated and the residue is acidified with 1 M HCl to pH 1 and extracted with EtOAc (3×). The org. layers are combined, dried (MgSO₄), filtered, and evaporated to yield 3-(((2R,5R,8S)-2,5-dibenzyl-8-isobutyl-6,9-dimethyl-4,7,10-trioxo-11-oxa-3,6,9-triazatetradec-13-en-1-yl)oxy)-6-fluoroquinoline-4-carboxylic acid (250 mg, 105%) which is used as such in the next step. LC-MS I: t_{R}=0.71/0.72 min; [M+H]⁺=713.42. A double peak visible by LC-MS indicates that epimerisation of one chiral centre occurred.

Step 3: HATU (96 mg, 0.25 mmol) is added to a RT soln. of 3-(((2R,5R,8S)-2,5-dibenzyl-8-isobutyl-6,9-dimethyl-4,7,10-trioxo-11-oxa-3,6,9-triazatetradec-13-en-1-yl)oxy)-6-fluoroquinoline-4-carboxylic acid (114 mg, 0.23 mmol), A-1.8 (78.1 mg, 0.24 mmol), and DIPEA (0.19 mL, 1.13 mmol) in DMF (4 mL) and the RM is stirred at RT for 16 h. Purification by prep. HPLC (acidic) yields tert-butyl (S)-3-(3-(((2R,5R,8S)-2,5-dibenzyl-8-isobutyl-6,9-dimethyl-4,7,10-trioxo-11-oxa-3,6,9-triazatetradec-13-en-1-yl)oxy)-6-fluoroquinoline-4-carboxamido)-4-((2-(3-methylisoxazol-5-yl)ethyl)amino)-4-oxobutanoate (187 mg, 56%). LC-MS B: t_{R}=1.21 min; [M+H]⁺=992.37.

Step 4: TFA (2 mL, 26 mmol) is added to a RT soln. of tert-butyl (S)-3-(3-(((2R,5R,8S)-2,5-dibenzyl-8-isobutyl-6,9-dimethyl-4,7,10-trioxo-11-oxa-3,6,9-triazatetradec-13-en-1-yl)oxy)-6-fluoroquinoline-4-carboxamido)-4-((2-(3-methylisoxazol-5-yl)ethyl)amino)-4-oxobutanoate (187 mg, 0.189 mmol) in DCM (3 mL) and the resulting mix. is stirred at RT for 2 h. The mix. is concentrated and co-evaporated with DCM (2×) to yield (S)-3-(3-(((2R,5R,8S)-2,5-dibenzyl-8-isobutyl-6,9-dimethyl-4,7,10-trioxo-11-oxa-3,6,9-triazatetradec-13-en-1-yl)oxy)-6-fluoroquinoline-4-carboxamido)-4-((2-(3-methylisoxazol-5-yl)ethyl)amino)-4-oxobutanoic acid (180 mg) which is used as such in the next step. LC-MS I: t_{R}=0.66/0.68 min; [M+H]⁺=936.3. A double peak visible by LC-MS indicates that epimerisation of one chiral centre occurred.

Step 5: Pd(Ph₃)₄ (22.3 mg, 0.019 mmol) is added to a RT soln. of S)-3-(3-(((2R,5R,8S)-2,5-dibenzyl-8-isobutyl-6,9-dimethyl-4,7,10-trioxo-11-oxa-3,6,9-triazatetradec-13-en-1-yl)oxy)-6-fluoroquinoline-4-carboxamido)-4-((2-(3-methylisoxazol-5-yl)ethyl)amino)-4-oxobutanoic acid (177 mg, 0.19 mmol) and 1,3-dimethylbarbituric acid (59.6 mg, 0.38 mmol) in DCM (1 mL) and the RM is stirred at RT for 1 h. The RM is concentrated to yield (S)-3-(3-((R)-2-((R)-2-((S)—N,4-dimethyl-2-(methylamino)pentanamido)-3-phenylpropanamido)-3-phenylpropoxy)-6-fluoroquinoline-4-carboxamido)-4-((2-(3-methylisoxazol-5-yl)ethyl)amino)-4-oxobutanoic acid (165 mg) which is used as such in the next step. LC-MS I: t_{R}=0.64 min; [M+H]⁺=852.59.

Step 6: The title compound is synthesised following the synthesis described for GM-5, step 7. LC-MS I: t_{R}=1.11 min; [M+H]⁺=834.77.

### General Method: GM-7

### Example 724: (3R,6RS,9S,13S)-3-Benzyl-6-((benzyloxy)methyl)-N-(2-(3-cyclopropyl-1,2,4-oxadiazol-5-yl)ethyl)-9-isobutyl-16-methoxy-7,10-dimethyl-5,8,11,15-tetraoxo-2,3,4,5,6,7,8,9,10,11,12,13,14,15-tetradecahydropyrido[3,4-p][1]oxa[4,7,10,14]tetraazacycloheptadecine-13-carboxamide

Step 1: HATU (1.49 g, 3.86 mmol) is added to a RT suspension of A-1.22 (1.25 g, 3.86 mmol), B-Acid-7 (1.55 g, 3.86 mmol), and DIPEA (2.0 mL, 11.6 mmol) in DCM (30 mL), then DMF (5 mL) is added to obtain a clear soln. The RM is stirred at RT overnight. The RM is concentrated and purified by FC (eluting with 0% to 100% EtOAc in hept) to yield tert-butyl (S)-3-(4-((R)-2-((tert-butoxycarbonyl)amino)-3-phenylpropoxy)-2-methoxynicotinamido)-4-((2-(3-cyclopropyl-1,2,4-oxadiazol-5-yl)ethyl)amino)-4-oxobutanoate (2.79 g, 100%). LC-MS I: t_{R}=1.12 min; [M+H]⁺=709.20.

Step 2: 4 M HCl in dioxane (2.0 mL, 8.0 mmol) is added to tert-butyl (S)-3-(4-((R)-2-((tert-butoxycarbonyl)amino)-3-phenylpropoxy)-2-methoxynicotinamido)-4-((2-(3-cyclopropyl-1,2,4-oxadiazol-5-yl)ethyl)amino)-4-oxobutanoate (2.79 g, 3.94 mmol) in DCM (30 mL) and MeOH (5 mL). The RM is stirred at RT for 48 h. The mix. is concentrated to yield tert-butyl (S)-3-(4-((R)-2-amino-3-phenylpropoxy)-2-methoxynicotinamido)-4-((2-(3-cyclopropyl-1,2,4-oxadiazol-5-yl)ethyl)amino)-4-oxobutanoate hydrochloride (2.55 g, 100%) as a yellow solid, which is used as such in the next step. LC-MS I: t_{R}=0.90 min; [M+H]⁺=609.35.

Step 3: HATU (128 mg, 0.33 mmol) is added to a RT soln. of tert-butyl (S)-3-(4-((R)-2-amino-3-phenylpropoxy)-2-methoxynicotinamido)-4-((2-(3-cyclopropyl-1,2,4-oxadiazol-5-yl)ethyl)amino)-4-oxobutanoate hydrochloride (304 mg, 0.33 mmol), D1-1.1 (96 mg, 0.30 mmol), and DIPEA (0.15 mL, 0.9 mmol) in DMF (3 mL) and the RM is stirred at RT for 15 min. Purification by prep. HPLC (basic) yields tert-butyl (S)-3-(4-((R)-2-(3-(benzyloxy)-2-((tert-butoxycarbonyl)(methyl)amino)propanamido)-3-phenylpropoxy)-2-methoxynicotinamido)-4-((2-(3-cyclopropyl-1,2,4-oxadiazol-5-yl)ethyl)amino)-4-oxobutanoate (108 mg, 40%) as a white solid. LC-MS I: t_{R}=1.23 min; [M+H]⁺=900.76.

Step 4: 4 M HCl in dioxane (0.06 mL, 0.24 mmol) is added to a RT soln. of tert-butyl (S)-3-(4-((R)-2-(3-(benzyloxy)-2-((tert-butoxycarbonyl)(methyl)amino)propanamido)-3-phenylpropoxy)-2-methoxynicotinamido)-4-((2-(3-cyclopropyl-1,2,4-oxadiazol-5-yl)ethyl)amino)-4-oxobutanoate (108 mg, 0.12 mmol) in MeOH (1 mL) and the RM is stirred at RT for 48 h. The RM is concentrated to yield tert-butyl (S)-3-(4-((R)-2-((RS)-3-(benzyloxy)-2-(methylamino)propanamido)-3-phenylpropoxy)-2-methoxynicotinamido)-4-((2-(3-cyclopropyl-1,2,4-oxadiazol-5-yl)ethyl)amino)-4-oxobutanoate hydrochloride (101 mg, 100%) as a white solid which is used as such in the next step. LC-MS I: t_{R}=1.05 min; [M+H]⁺=800.64.

Step 5: HATU (56.4 mg, 0.14 mmol) is added to a RT soln. of tert-butyl (S)-3-(4-((R)-2-(3-(benzyloxy)-2-(methylamino)propanamido)-3-phenylpropoxy)-2-methoxynicotinamido)-4-((2-(3-cyclopropyl-1,2,4-oxadiazol-5-yl)ethyl)amino)-4-oxobutanoate hydrochloride (100 mg, 0.12 mmol), Boc-N-methyl-L-leucine (36.4 mg, 0.14 mmol) and DIPEA (0.08 mL, 0.48 mmol) in DMF (1.2 mL) and the RM is stirred at RT for 15 min. Purification by prep. HPLC (basic) yields tert-butyl (S)-3-(4-(((6S,9RS,12R)-12-benzyl-9-((benzyloxy)methyl)-6-isobutyl-2,2,5,8-tetramethyl-4,7,10-trioxo-3-oxa-5,8,11-triazatridecan-13-yl)oxy)-2-methoxynicotinamido)-4-((2-(3-cyclopropyl-1,2,4-oxadiazol-5-yl)ethyl)amino)-4-oxobutanoate (60 mg, 49%) as a white solid. LC-MS I: t_{R}=1.38 min; [M+H]⁺=1027.94.

Step 6: TFA (0.6 mL, 7.83 mmol) is added to a RT soln. of tert-butyl (S)-3-(4-(((6S,9RS,12R)-12-benzyl-9-((benzyloxy)methyl)-6-isobutyl-2,2,5,8-tetramethyl-4,7,10-trioxo-3-oxa-5,8,11-triazatridecan-13-yl)oxy)-2-methoxynicotinamido)-4-((2-(3-cyclopropyl-1,2,4-oxadiazol-5-yl)ethyl)amino)-4-oxobutanoate (60.3 mg, 0.06 mmol) in DCM (1 mL) and the resulting mix. is stirred at RT for 1 h. The mix. is concentrated and co-evaporated with DCM (2×) to yield (S)-3-(4-((R)-2-(3-(benzyloxy)-2-((S)—N,4-dimethyl-2-(methylamino)pentanamido)propanamido)-3-phenylpropoxy)-2-methoxynicotinamido)-4-((2-(3-cyclopropyl-1,2,4-oxadiazol-5-yl)ethyl)amino)-4-oxobutanoic acid (55 mg, 100%) which is used as such in the next step. LC-MS I: t_{R}=0.64 min; [M+H]⁺=871.79.

Step 7: HATU (23 mg, 0.046 mmol) is added to a RT soln. of (S)-3-(4-((R)-2-(3-(benzyloxy)-2-((S)—N,4-dimethyl-2-(methylamino)pentanamido)propanamido)-3-phenylpropoxy)-2-methoxynicotinamido)-4-((2-(3-cyclopropyl-1,2,4-oxadiazol-5-yl)ethyl)amino)-4-oxobutanoic acid (51.1 mg, 0.06 mmol) and DIPEA (50 NL, 0.29 mmol) in DMF (1 mL) and the RM is stirred at RT for 30 min. Purification by prep. HPLC (basic) yields the title compound (29.3 mg, 82%) as pale yellow solid. LC-MS 036: t_{R}=1.08 min; [M+H]⁺=853.34.

### General Method: GM-8

### Example 725: (4aR,7R,18S,22S)-7-Benzyl-22-isobutyl-21-methyl-N-(2-(3-methylisoxazol-5-yl)ethyl)-5,16,20,23-tetraoxo-2,3,4,4a,5,6,7,8,16,17,18,19,20,21,22,23-hexadecahydro-1H-naphtho[1,2-p]pyrazino[2,1-f][1]oxa[4,7,10,14]tetraazacycloheptadecine-18-carboxamide

Step 1: HATU (1.86 g, 4.89 mmol) is added to a RT soln. of C-7.1 (2.48 g, 4.65 mmol), B-1.13 (2.37 g, 4.65 mmol), and DIPEA (2.4 mL, 14 mmol) in MeCN (14.5 mL) and the RM is stirred at RT for 30 min. Water (10 mL) and DCM (100 mL) are added to the RM and the layers are separated. The aq. layer is extracted with DCM (2×75 mL) and the combined org. layers are washed with brine (50 mL), dried (MgSO₄), filtered, and concentrated. Purification by FC (eluting with 15-45% EtOAc/hept, R_{f}=0.45 in EtOAc/hept 3:7) yields 2,2,2-trichloroethyl (R)-3-(((R)-1-((1-((benzyloxy)carbonyl)naphthalen-2-yl)oxy)-3-phenylpropan-2-yl)carbamoyl)-4-(N-(tert-butoxycarbonyl)-N-methyl-L-leucyl)piperazine-1-carboxylate (2.25 g, 52%) as a white foam. LC-MS B: t_{R}=1.33 min; [M+H]⁺=927.11.

Step 2: Pd/C (129 mg, 0.121 mmol) is added to a RT soln. (degassed) of 2,2,2-trichloroethyl (R)-3-(((R)-1-((1-((benzyloxy)carbonyl)naphthalen-2-yl)oxy)-3-phenylpropan-2-yl)carbamoyl)-4-(N-(tert-butoxycarbonyl)-N-methyl-L-leucyl)piperazine-1-carboxylate (2.24 g, 2.42 mmol) in MeOH (20 mL). The RM is stirred at RT for 18 h under a H₂ atm. The mix. is filtered and the filtrate concentrated to yield 2-((R)-2-((R)-1-(N-(tert-butoxycarbonyl)-N-methyl-L-leucyl)-4-((2,2,2-trichloroethoxy)carbonyl)piperazine-2-carboxamido)-3-phenylpropoxy)-1-naphthoic acid (2.01 g, 100%) as a white foam which is used as such in the next step. LC-MS B: t_{R}=1.21 min; [M+H]⁺=836.91.

Step 3: HATU (355 mg, 0.933 mmol) is added to a RT soln. of 2-((R)-2-((R)-1-(N-(tert-butoxycarbonyl)-N-methyl-L-leucyl)-4-((2,2,2-trichloroethoxy)carbonyl)piperazine-2-carboxamido)-3-phenylpropoxy)-1-naphthoic acid (600 mg, 0.718 mmol), A-1.8 (256 mg, 0.86 mmol), and DIPEA (0.369 mL, 2.15 mmol) in MeCN (6 mL) and the RM is stirred at RT for 1 h. Water (8 mL) and DCM (80 mL) are added to the RM, then the layers are separated. The aq. layer is extracted with DCM (2×75 mL) and the combined org. layers are washed with brine (10 mL), dried (MgSO₄), filtered, and concentrated. Purification by FC (eluting with 20% to 60% EtOAc in hept, with R_{f}=0.26 in EtOAc/hept 1:1) yields 2,2,2-trichloroethyl (R)-3-(((R)-1-((1-(((S)-4-(tert-butoxy)-1-((2-(3-methylisoxazol-5-yl)ethyl)amino)-1,4-dioxobutan-2-yl)carbamoyl)naphthalen-2-yl)oxy)-3-phenylpropan-2-yl)carbamoyl)-4-(N-(tert-butoxycarbonyl)-N-methyl-L-leucyl)piperazine-1-carboxylate (512 mg, 64%) as a white foam. LC-MS B: t_{R}=1.30 min; [M+H]⁺=1116.18.

Step 4: TFA (1.73 mL, 22.4 mmol) is added to a RT soln. of 2,2,2-trichloroethyl (R)-3-(((R)-1-((1-(((S)-4-(tert-butoxy)-1-((2-(3-methylisoxazol-5-yl)ethyl)amino)-1,4-dioxobutan-2-yl)carbamoyl)naphthalen-2-yl)oxy)-3-phenylpropan-2-yl)carbamoyl)-4-(N-(tert-butoxycarbonyl)-N-methyl-L-leucyl)piperazine-1-carboxylate (500 mg, 0.448 mmol) in DCM (5 mL) and the RM is stirred at RT for 3 h. The mix. is concentrated and co-evaporated with DCM before being dried under HV. LC-MS B t_{R}=0.89 min; [M+H]⁺=960.02. The dried residue is taken up in MeCN (5 mL), then DIPEA (0.384 mL, 2.24 mmol) and HATU (256 mg, 0.672 mmol) are added and the RM is stirred at RT for 30 min. Water (10 mL) and DCM (100 mL) are added to the RM and the layers are separated. The aq. layer is extracted with DCM (2×75 mL) and the combined org. layers are washed with brine (50 mL), dried (MgSO₄), filtered, and concentrated. Purification by FC (eluting with 35% to 100% EtOAc in hept, R_{f}=0.28 in EtOAc/hept 7:3) yields 2,2,2-trichloroethyl (4aR,7R,18S,22S)-7-benzyl-22-isobutyl-21-methyl-18-((2-(3-methylisoxazol-5-yl)ethyl)carbamoyl)-5,16,20,23-tetraoxo-1,2,4,4a,5,6,7,8,16,17,18,19,20,21,22,23-hexadecahydro-3H-naphtho[1,2-p]pyrazino[2,1-f][1]oxa[4,7,10,14]tetraazacycloheptadecine-3-carboxylate (293 mg, 69%) as a white foam. LC-MS B: t_{R}=1.13 min; [M+H]⁺=940.04.

Step 5: Zinc powder (176 mg, 2.69 mmol) is added to a RT soln. of 2,2,2-trichloroethyl (4aR,7R,18S,22S)-7-benzyl-22-isobutyl-21-methyl-18-((2-(3-methylisoxazol-5-yl)ethyl)carbamoyl)-5,16,20,23-tetraoxo-1,2,4,4a,5,6,7,8,16,17,18,19,20,21,22,23-hexadecahydro-3H-naphtho[1,2-p]pyrazino[2,1-f][1]oxa[4,7,10,14]tetraazacycloheptadecine-3-carboxylate (260 mg, 0.269 mmol) and AcOH (0.339 mL, 5.92 mmol) in DCM (5 mL) and the RM is stirred at RT for 1 h. To the RM is added sat. NaHCO₃ (10 mL) and DCM (50 mL), and the layers are separated. The aq. layer is extracted with DCM (2×25 mL) and the combined org. layers are washed with brine (10 mL), dried (MgSO₄), filtered, and concentrated to yield the title compound (215 mg, 104%) as a white foam. LC-MS B: t_{R}=0.80 min; [M+H]⁺=765.67.

### General Method: GM-9

### Example 761: (8′R,14'S,18'S)-8′-Benzyl-14′-isobutyl-2′,12′,15′-trimethyl-10′,13′,16′,20′-tetraoxo-N-(2-(3-phenylisoxazol-5-yl)ethyl)-7′,8′,9′,10′,13′,14′,15′,16′,17′,18′,19′,20′-dodecahydro-12′H-spiro[cyclopropane-1,11′-oxazolo[4′,5′:5,6]benzo[1,2-p][1]oxa[4,7,10,14]tetraazacycloheptadecine]-18′-carboxamide

Step 1: Methyl 6-((R)-2-(1-((S)-2-((tert-butoxycarbonyl)(methyl)amino)-N,4-dimethylpentanamido)cyclopropane-1-carboxamido)-3-phenylpropoxy)-2-methylbenzo[d]oxazole-7-carboxylate is prepared from B-2.5 (302 mg, 0.65 mmol) and C-1.2 (221 mg, 0.65 mmol) in analogy to the procedure described for Example 222, step 1. LC-MS J: t_{R}=2.30 min; [M+H]⁺=665.3.

Step 2: A RT soln. of methyl 6-((R)-2-(1-((S)-2-((tert-butoxycarbonyl)(methyl)amino)-N,4-dimethylpentanamido)cyclopropane-1-carboxamido)-3-phenylpropoxy)-2-methylbenzo[d]oxazole-7-carboxylate (315 mg, 0.47 mmol) and 2M aq. NaOH (4.74 mL, 9.48 mmol) in MeOH (10 mL) is stirred for 16 h. The RM is concentrated in vacuo and the residue is partitioned between water and EtOAc and the layers are separated. The aq. phase is re-extracted with EtOAc (2×) and the combined org. extracts are washed with brine, dried over Na₂SO₄, filtered and evaporated in vacuo to give 6-((R)-2-(1-((S)-2-((tert-butoxycarbonyl)(methyl)amino)-N,4-dimethylpentanamido)cyclopropane-1-carboxamido)-3-phenylpropoxy)-2-methylbenzo[d]oxazole-7-carboxylic acid as a white solid. LC-MS J: t_{R}=1.82 min; [M+H]⁺=651.2.

Steps 3&4: (8′R,14'S,18'S)-8′-benzyl-N-(but-3-yn-1-yl)-14′-isobutyl-2′,12′,15′-trimethyl-10′,13′,16′,20′-tetraoxo-7′,8′,9′,10′,13′,14′,15′,16′,17′,18′,19′,20′-dodecahydro-12′H-spiro[cyclopropane-1,11′-oxazolo[4′,5′:5,6]benzo[1,2-p][1]oxa[4,7,10,14]tetraazacycloheptadecine]-18′-carboxamide is prepared from 6-((R)-2-(1-((S)-2-((tert-butoxycarbonyl)(methyl)amino)-N,4-dimethylpentanamido)cyclopropane-1-carboxamido)-3-phenylpropoxy)-2-methylbenzo[d]oxazole-7-carboxylic acid and A-1.33 in analogy to the sequence of reactions as described for Example 222, steps 3-4. LC-MS J: t_{R}=1.97 min; [M+H]⁺=699.2.

Step 5: NCS (64 mg, 0.48 mmol) is added to a RT soln. of (E)-benzaldehyde oxime (48 mg, 0.40 mmol) in DMF (0.5 mL) and the RM is stirred for 16 h. The RM is partitioned between water and Et₂O and the layers are separated. The aq. phase is re-extracted with Et₂O (2×) and the combined org. extracts are washed with brine and dried over a stream of air to give (Z)—N-hydroxybenzimidoyl chloride. Note: Compound is unstable and is used directly in the next step.

Step 6: A RT mix. of (8′R,14'S,18'S)-8′-benzyl-N-(but-3-yn-1-yl)-14′-isobutyl-2′,12′,15′-trimethyl-10′,13′,16′,20′-tetraoxo-7′,8′,9′,10′,13′,14′,15′,16′,17′,18′,19′,20′-dodecahydro-12′H-spiro[cyclopropane-1,11′-oxazolo[4′,5′:5,6]benzo[1,2-p][1]oxa[4,7,10,14]tetraazacycloheptadecine]-18′-carboxamide (45 mg, 0.06 mmol), (Z)—N-hydroxybenzimidoyl chloride (30 mg, 0.19 mmol), CuI (0.2 mg, 1.29 μmol), and K₂CO₃ (36 mg, 0.26 mmol) in THE (1 mL) is stirred for 48 h. The RM is concentrated in vacuo and re-dissolved in DMF before being directly purified by prep. HPLC (basic) to give the title compound as a white solid. LC-MS H: t_{R}=1.19 min; [M+H]⁺=818.8.

### Example 762: (8′R,14'S,18'S)-8′-Benzyl-14′-isobutyl-N-(2-(3-isopropylisoxazol-5-yl)ethyl)-2′,12′,15′-trimethyl-10′,13′,16′,20′-tetraoxo-7′,8′,9′,10′,13′,14′,15′,16′,17′,18′,19′,20′-dodecahydro-12′H-spiro[cyclopropane-1,11′-oxazolo[4′,5′:5,6]benzo[1,2-p][1]oxa[4,7,10,14]tetraazacycloheptadecine]-18′-carboxamide

The title compound is prepared from (8′R,14'S,18'S)-8′-benzyl-N-(but-3-yn-1-yl)-14′-isobutyl-2′,12′,15′-trimethyl-10′,13′,16′,20′-tetraoxo-7′,8′,9′,10′,13′,14′,15′,16′,17′,18′,19′,20′-dodecahydro-12′H-spiro[cyclopropane-1,11′-oxazolo[4′,5′:5,6]benzo[1,2-p][1]oxa[4,7,10,14]tetraazacycloheptadecine]-18′-carboxamide (Ex. 761, steps 1-4) and (Z)—N-hydroxyisobutyrimidoyl chloride (prepared from (E)-isobutyraldehyde oxime, see Ex. 761, step 5) in analogy to the procedure described for Ex. 761, step 6. LC-MS H: t_{R}=1.14 min; [M+H]⁺=784.6.

### Example 763: (3'S,7'S,13′R)-13′-Benzyl-N-(2-(3-benzyl-1,2,4-oxadiazol-5-yl)ethyl)-7′-isobutyl-6′,9′-dimethyl-1′,5′,8′,11′-tetraoxo-2′,3′,4′,5′,6′,7′,8′,9′,11′,12′,13′,14′-dodecahydro-1′H-spiro[cyclopropane-1,10′-[1]oxa[4,7,10,14]tetraazacycloheptadecino[17,16-c]quinoline]-3′-carboxamide

Steps 1-4: Benzyl 3-((3'S,7'S,13′R)-13′-benzyl-7′-isobutyl-6′,9′-dimethyl-1′,5′,8′,11′-tetraoxo-2′,3′,4′,5′,6′,7′,8′,9′,11′,12′,13′,14′-dodecahydro-1′H-spiro[cyclopropane-1,10′-[1]oxa[4,7,10,14]tetraazacycloheptadecino[17,16-c]quinoline]-3′-carboxamido)propanoate is prepared from B-1.14, C-1.2, and A-1.34 in analogy to the sequence of reactions described for Ex. 222, steps 1-4. LC-MS J: t_{R}=2.07 min; [M+H]⁺=805.4.

Step 5: 3-((3'S,7'S,13′R)-13′-Benzyl-7′-isobutyl-6′,9′-dimethyl-1′,5′,8′,11′-tetraoxo-2′,3′,4′,5′,6′,7′,8′,9′,11′,12′,13′,14′-dodecahydro-1′H-spiro[cyclopropane-1,10′-[1]oxa[4,7,10,14]tetraazacycloheptadecino[17,16-c]quinoline]-3′-carboxamido)propanoic acid is prepared from benzyl 3-((3'S,7'S,13′R)-13′-benzyl-7′-isobutyl-6′,9′-dimethyl-1′,5′,8′,11′-tetraoxo-2′,3′,4′,5′,6′,7′,8′,9′,11′,12′,13′,14′-dodecahydro-1′H-spiro[cyclopropane-1,10′-[1]oxa[4,7,10,14]tetraazacycloheptadecino[17,16-c]quinoline]-3′-carboxamido)propanoate following the hydrogenation procedure described for Ex. 222, step 2. LC-MS J: t_{R}=1.63 min; [M+H]⁺=715.3.

Step 6: Hydroxylamine.HCl (1.19 g, 17.1 mmol) is added to a RT soln. of benzyl cyanide (1.0 mL, 8.5 mmol) in MeOH (20 mL) followed by a soln. of NaOH (0.68 g, 17.1 mmol)) in water (5 mL) and the RM is stirred for 16 h. The RM is concentrated in vacuo and the residue is co-evaporated with PhMe (2×) to give N′-hydroxy-2-phenylacetimidamide as a colourless oil. Note: Used directly as such in the next step.

Step 7: HATU (38 mg, 0.10 mmol) is added to a RT soln. of 3-((3'S,7'S,13′R)-13′-benzyl-7′-isobutyl-6′,9′-dimethyl-1′,5′,8′,11′-tetraoxo-2′,3′,4′,5′,6′,7′,8′,9′,11′,12′,13′,14′-dodecahydro-1′H-spiro[cyclopropane-1,10′-[1]oxa[4,7,10,14]tetraazacycloheptadecino[17,16-c]quinoline]-3′-carboxamido)propanoic acid (50 mg, 0.07 mmol) and DIPEA (35 μL, 0.20 mmol) in NMP (1 mL) and the RM is stirred for 10 min before a soln. of N′-hydroxy-2-phenylacetimidamide (33 mg, 0.22 mmol) in NMP (200 μL) is added and stirring is continued for 30 min. The RM is then heated to 80° C. for 16 h. The RM is directly purified by prep. HPLC (basic) to give the title compound as a white solid. LC-MS H: t_{R}=1.22 min; [M+H]⁺=829.8.

### Example 764: (3'S,7'S,13′R)-13′-Benzyl-N-(2-(3-(cyclopropylmethyl)-1,2,4-oxadiazol-5-yl)ethyl)-7′-isobutyl-6′,9′-dimethyl-1′,5′,8′,11′-tetraoxo-2′,3′,4′,5′,6′,7′,8′,9′,11′,12′,13′,14′-dodecahydro-1′H-spiro[cyclopropane-1,10′-[1]oxa[4,7,10,14]tetraazacycloheptadecino[17,16-c]quinoline]-3′-carboxamide

Step 1: Hydroxylamine 50% aq. soln. (0.19 mL, 1.48 mmol) is added to a RT soln. of 2-cyclopropylacetonitrile (100 mg, 1.23 mmol) in EtOH (1.5 mL) and the RM is heated to reflux for 24 h. The RM is concentrated in vacuo and the residue is co-evaporated with PhMe (2×) to give 2-cyclopropyl-N′-hydroxyacetimidamide as a colourless oil. Note: Used directly as such in the next step.

Steps 2-7: The title compound is prepared from 3-((3'S,7'S,13′R)-13′-benzyl-7′-isobutyl-6′,9′-dimethyl-1′,5′,8′,11′-tetraoxo-2′,3′,4′,5′,6′,7′,8′,9′,11′,12′,13′,14′-dodecahydro-1′H-spiro[cyclopropane-1,10′-[1]oxa[4,7,10,14]tetraazacycloheptadecino[17,16-c]quinoline]-3′-carboxamido)propanoic acid (Ex. 763, steps 1-5) and 2-cyclopropyl-N′-hydroxyacetimidamide in analogy to the procedure described for Ex. 763, step 7. LC-MS H: t_{R}=1.17 min; [M+H]⁺=793.7.

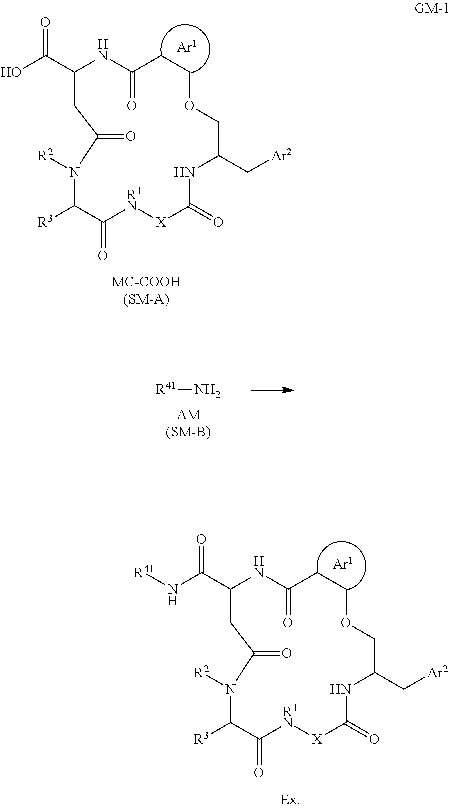

Listed in Table MC-1 below are compounds of general formula (I) that are prepared from their corresponding building blocks MC-COOH (SM-A) and amine (SM-B) as described above in General Method 1 (GM-1). In the following tables * denotes an example compound isolated during the synthesis, most often separated by prep. HPLC purification of the final synthetic step as a minor epimer due to epimerisation of a chiral centre. In certain cases, an enantiomerically or diastereomerically pure building block(s) undergoes epimersation during the synthesis and the example compound is isolated as a mixture of epimers.

TABLE MC-1
(GM-1)

| | | | t_{R} [min] | MS-data |
|---|---|---|---|---|
| | SM-A | SM-B | LC-MS | m/z |
| Ex. | MC-COOH | AM | Method | [M + H]⁺ |
| 1 | MC-COOH-8 | AM-1.1 | 1.14 (I) | 828.23 |
| 2 | MC-COOH-9 | AM-1.1 | 1.23 (D) | 854.37 |
| 3 | MC-COOH-2 | AM-2.1 | 1.00 (I) | 786.23 |
| 4 | MC-COOH-2 | AM-2.2 | 0.99 (I) | 770.2 |
| 5 | MC-COOH-10 | AM-1.1 | 1.12 (D) | 848.27 |
| 6 | MC-COOH-11 | AM-1.1 | 1.14 (D) | 892.29 |
| 7 | MC-COOH-12 | AM-1.1 | 1.00 (B) | 770.46 |
| 8 | MC-COOH-2 | AM-2.3 | 0.98 (D) | 801.37 |
| 9 | MC-COOH-13 | AM-1.1 | 1.13 (B) | 868.35 |
| 10 | MC-COOH-14 | AM-1.1 | 1.19 (B) | 896.38 |
| 11 | MC-COOH-15 | AM-2.1 | 1.27 (I) | 852.27 |
| 12 | MC-COOH-15 | AM-3.1 | 1.24 (I) | 868.28 |
| 13 | MC-COOH-15 | AM-1.2 | 1.20/1.22 (I) | 838.25 |
| 14 | MC-COOH-15 | AM-2.2 | 1.26 (I) | 836.24 |
| 15 | MC-COOH-15 | AM-2.5 | 1.24 (I) | 838.25 |
| 16 | MC-COOH-15 | AM-1.3 | 1.21/1.22 (I) | 837.22 |
| 17 | MC-COOH-3 | AM-1.4 | 1.06 (I) | 794.64 |
| 18 | MC-COOH-15 | AM-1.42 | 1.11 (I) | 798.49 |
| 19 | MC-COOH-15 | AM-1.43 | 1.09 (I) | 798.5 |
| 20 | MC-COOH-15 | AM-1.44 | 1.09 (I) | 798.47 |
| 21 | MC-COOH-15 | AM-1.45 | 1.19 (I) | 800.56 |
| 22 | MC-COOH-15 | AM-1.46 | 1.14 (I) | 794.56 |
| 23 | MC-COOH-3 | AM-1.47 | 0.97 (I) | 785.28 |
| 24 | MC-COOH-3 | AM-1.48 | 0.97 (I) | 785.28 |
| 25 | MC-COOH-3 | AM-1.5 | 1.04 (I) | 794.32 |
| 26 | MC-COOH-3 | AM-1.57 | 1.07 (I) | 830.22 |
| 27 | MC-COOH-3 | AM-1.6 | 1.06 (I) | 764.32 |
| 28 | MC-COOH-3 | AM-2.8 | 1.04 (I) | 831.34 |
| 29 | MC-COOH-3 | AM-1.49 | 1.05 (I) | 811.63 |
| 30 | MC-COOH-3 | AM-1.50 | 1.05 (I) | 805.67 |
| 31 | MC-COOH-3 | AM-2.7 | 1.06 (I) | 831.68 |
| 32 | MC-COOH-4 | AM-1.51 | 1.09 (I) | 820.61 |
| 33 | MC-COOH-4 | AM-1.52 | 1.08 (I) | 808.38 |
| 34 | MC-COOH-4 | AM-1.53 | 1.07 (I) | 808.38 |
| 35 | MC-COOH-4 | AM-11.1 | 1.06 (I) | 850.42 |
| 36 | MC-COOH-4 | AM-12.1 | 1.16 (I) | 868.42 |
| 37 | MC-COOH-4 | AM-12.2 | 1.11 (I) | 854.38 |
| 38 | MC-COOH-4 | AM-1.50 | 1.06 (B) | 803.39 |
| 39 | MC-COOH-4 | AM-2.6 | 1.04 (I) | 820.39 |
| 40 | MC-COOH-4 | AM-1.54 | 1.03 (I) | 787.41 |
| 41 | MC-COOH-4 | AM-2.8 | 1.07 (I) | 829.31 |
| 42 | MC-COOH-4 | AM-2.5 | 1.06 (I) | 804.39 |
| 43 | MC-COOH-4 | AM-1.55 | 0.99 (I) | 781.29 |
| 44 | MC-COOH-4 | AM-1.25 | 1.00 (B) | 782.26 |
| 45 | MC-COOH-4 | AM-1.26 | 1.02 (B) | 794.43 |
| 46 | MC-COOH-5 | AM-1.56 | 1.01 (I) | 812.24 |
| 47 | MC-COOH-5 | AM-1.10 | 1.1 (I) | 791.27 |
| 48 | MC-COOH-5 | AM-1.11 | 1.18 (I) | 819.32 |
| 49 | MC-COOH-5 | AM-1.12 | 1.08 (I) | 841.18 |
| 50 | MC-COOH-5 | AM-1.13 | 1.01 (I) | 781.16 |
| 51 | MC-COOH-5 | AM-1.14 | 1.06 (I) | 797.12 |
| 52 | MC-COOH-5 | AM-1.15 | 1.07 (I) | 841.14 |
| 53 | MC-COOH-5 | AM-2.6 | 0.96 (I) | 821.25 |
| 54 | MC-COOH-5 | AM-2.5 | 0.98 (I) | 805.26 |
| 55 | MC-COOH-5 | AM-2.7 | 0.99 (I) | 830.17 |
| 56 | MC-COOH-5 | AM-2.8 | 0.98 (I) | 830.16 |
| 57 | MC-COOH-5 | AM-1.7 | 0.99 (I) | 823.32 |
| 58 | MC-COOH-5 | AM-1.6 | 1.01 (I) | 763.28 |
| 59 | MC-COOH-5 | AM-4.1 | 1.01 (I) | 811.26 |
| 60 | MC-COOH-5 | AM-1.8 | 1.05 (I) | 777.28 |
| 61 | MC-COOH-5 | AM-1.16 | 1.09 (I) | 791.3 |
| 62 | MC-COOH-5 | AM-1.9 | 1.05 (I) | 777.28 |
| 63 | MC-COOH-5 | AM-1.17 | 1.1 (I) | 791.3 |
| 64 | MC-COOH-5 | AM-1.18 | 1.05 (I) | 777.28 |
| 65 | MC-COOH-5 | AM-1.19 | 1.00 (I) | 823.29 |
| 66 | MC-COOH-5 | AM-1.20 | 1.01 (I) | 781.17 |
| 67 | MC-COOH-5 | AM-1.21 | 1.02 (I) | 781.16 |
| 68 | MC-COOH-5 | AM-1.22 | 1.03 (I) | 799.25 |
| 69 | MC-COOH-5 | AM-1.23 | 1.01 (I) | 831.25 |
| 70 | MC-COOH-4 | AM-5.1 | 1.03 (I) | 781.51 |
| 71 | MC-COOH-1 | AM-1.7 | 1.08 (I) | 821.36 |
| 72 | MC-COOH-1 | AM-1.22 | 1.12 (I) | 797.15 |
| 73 | MC-COOH-1 | AM-6.1 | 1.15 (I) | 815.31 |
| 74 | MC-COOH-1 | AM-4.3 | 1.14 (I) | 827.3 |
| 75 | MC-COOH-1 | AM-4.4 | 1.13 (I) | 797.18 |
| 76 | MC-COOH-1 | AM-1.23 | 1.1 (1) | 829.23 |
| 77 | MC-COOH-1 | AM-1.6 | 1.11 (I) | 761.3 |
| 78 | MC-COOH-1 | AM-1.24 | 0.95 (I) | 767.29 |
| 79 | MC-COOH-1 | AM-1.25 | 1.00 (I) | 781.22 |
| 80 | MC-COOH-1 | AM-1.26 | 1.02 (I) | 793.34 |
| 81 | MC-COOH-1 | AM-1.19 | 1.09 (I) | 821.41 |
| 82 | MC-COOH-1 | AM-1.27 | 0.99 (I) | 766.04 |
| 83 | MC-COOH-1 | AM-1.28 | 0.92 (I) | 765.21 |
| 84 | MC-COOH-1 | AM-1.5 | 1.09 (I) | 791.31 |
| 85 | MC-COOH-1 | AM-1.29 | 1.1 (I) | 809.37 |
| 86 | MC-COOH-1 | AM-7.2 | 1.14 (B) | 857.37 |
| 87 | MC-COOH-1 | AM-7.3 | 1.14 (B) | 913.34 |
| 88 | MC-COOH-1 | AM-8.1 | 1.02 (B) | 912.5 |
| 89 | MC-COOH-1 | AM-1.30 | 1.17 (I) | 808.81 |
| 90* | MC-COOH-1 | AM-1.30 | 1.12 (I) | 808.75 |
| 91 | MC-COOH-1 | AM-2.17 | 1.09 (I) | 809.26 |
| 92 | MC-COOH-1 | AM-2.18 | 1.15 (I) | 793.22 |
| 93 | MC-COOH-1 | AM-9.1 | 1.03 (I) | 858.29 |
| 94 | MC-COOH-1 | AM-2.20 | 1.13 (I) | 797.06 |
| 95 | MC-COOH-1 | AM-2.19 | 1.14 (I) | 805.28 |
| 96 | MC-COOH-1 | AM-1.9 | 1.15 (I) | 775.22 |
| 97 | MC-COOH-1 | AM-2.16 | 1.12 (I) | 797.15 |
| 98 | MC-COOH-1 | AM-1.31 | 0.99 (I) | 765.98 |
| 99 | MC-COOH-1 | AM-4.2 | 1.22 (I) | 875.17 |
| 100 | MC-COOH-1 | AM-2.15 | 1.08 (I) | 809.23 |
| 101 | MC-COOH-16 | AM-3.1 | 0.99 (I) | 785.90 |
| 102 | MC-COOH-1 | AM-2.14 | 1.17 (I) | 811.24 |
| 103 | MC-COOH-1 | AM-1.32 | 0.98 (I) | 768.14 |
| 104 | MC-COOH-1 | AM-2.13 | 1.16 (I) | 793.25 |
| 105 | MC-COOH-1 | AM-1.33 | 1.04 (I) | 792.25 |
| 106 | MC-COOH-5 | AM-1.33 | 0.97 (I) | 794.61 |
| 107 | MC-COOH-5 | AM-1.30 | 1.06 (I) | 810.62 |
| 108 | MC-COOH-7 | AM-1.6 | 1.08 (I) | 747.6 |
| 109 | MC-COOH-7 | AM-1.7 | 1.05 (I) | 807.65 |
| 110 | MC-COOH-7 | AM-1.22 | 1.09 (I) | 783.58 |
| 111 | MC-COOH-7 | AM-1.5 | 1.05 (I) | 777.59 |
| 112 | MC-COOH-7 | AM-1.9 | 1.12 (I) | 761.6 |
| 113 | MC-COOH-7 | AM-4.4 | 1.10 (I) | 783.56 |
| 114 | MC-COOH-7 | AM-1.25 | 0.96 (I) | 767.59 |
| 115 | MC-COOH-7 | AM-1.26 | 0.98 (I) | 779.59 |
| 116 | MC-COOH-7 | AM-1.29 | 1.07 (I) | 795.63 |
| 117 | MC-COOH-7 | AM-1.31 | 0.96 (I) | 752.55 |
| 118 | MC-COOH-7 | AM-1.33 | 1.01 (I) | 778.63 |
| 119 | MC-COOH-7 | AM-1.30 | 1.10 (I) | 794.66 |
| 120 | MC-COOH-7 | AM-1.34 | 1.10 (I) | 783.58 |
| 121 | MC-COOH-7 | AM-1.35 | 0.99 (I) | 779.62 |
| 122 | MC-COOH-1 | AM-2.12 | 1.05 (I) | 839.3 |
| 123 | MC-COOH-1 | AM-2.11 | 1.03 (I) | 839.29 |
| 124 | MC-COOH-1 | AM-2.10 | 0.97 (I) | 791.27 |
| 125 | MC-COOH-1 | AM-2.9 | 1.10 (I) | 842.32 |
| 126 | MC-COOH-1 | AM-1.34 | 1.16 (I) | 797.48 |
| 127 | MC-COOH-1 | AM-1.35 | 1.06 (I) | 793.51 |
| 128 | MC-COOH-1 | AM-1.36 | 1.10 (I) | 807.56 |
| 129 | MC-COOH-1 | AM-1.37 | 1.00 (I) | 779.55 |
| 130 | MC-COOH-5 | AM-7.1 | 1.01 (I) | 861.95 |
| 131 | MC-COOH-6 | AM-1.4 | 1.09 (I) | 795.52 |
| 132 | MC-COOH-6 | AM-1.22 | 1.11 (I) | 801.5 |
| 133 | MC-COOH-6 | AM-10.1 | 1.03 (I) | 843.54 |
| 134 | MC-COOH-6 | AM-10.2 | 1.02 (I) | 843.54 |
| 135 | MC-COOH-6 | AM-2.7 | 1.09 (I) | 832.56 |
| 136 | MC-COOH-6 | AM-2.8 | 1.08 (I) | 832.54 |
| 137 | MC-COOH-6 | AM-2.12 | 1.05 (I) | 843.55 |
| 138 | MC-COOH-6 | AM-2.11 | 1.03 (I) | 843.56 |
| 139 | MC-COOH-6 | AM-2.10 | 0.96 (I) | 795.57 |
| 140 | MC-COOH-6 | AM-1.35 | 1.02 (I) | 797.37 |
| 141 | MC-COOH-6 | AM-1.31 | 0.98 (I) | 770.52 |
| 142 | MC-COOH-6 | AM-1.33 | 1.03 (I) | 796.55 |
| 143 | MC-COOH-6 | AM-1.28 | 0.91 (I) | 769.53 |
| 144 | MC-COOH-6 | AM-1.26 | 1.00 (I) | 797.41 |
| 145 | MC-COOH-7 | AM-1.38 | 1.10 (I) | 783.54 |
| 146 | MC-COOH-6 | AM-1.39 | 0.95 (I) | 771.46 |
| 147 | MC-COOH-6 | AM-1.40 | 1.00 (B) | 770.08 |
| 148 | MC-COOH-2 | AM-2.4 | 1.00 (I) | 786.58 |
| 149 | MC-COOH-15 | AM-2.6 | 1.22 (I) | 854.25 |
| 150 | MC-COOH-15 | AM-1.57 | 1.27 (I) | 862.14 |
| 151 | MC-COOH-16 | AM-1.19 | 0.99 (I) | 774.16 |
| 152 | MC-COOH-2 | AM-1.73 | 0.96 (I) | 772.18 |
| 153 | MC-COOH-16 | AM-1.5 | 1.15 (H) | 744.7 |
| 154 | MC-COOH-17 | AM-1.1 | 1.03 (B) | 856.32 |
| 155 | MC-COOH-18 | AM-1.1 | 1.25 (H) | 872.7 |
| 156 | MC-COOH-19 | AM-1.1 | 1.21 (H) | 860.9 |
| 157 | MC-COOH-15 | AM-1.59 | 1.33 (I) | 832.92 |
| 158 | MC-COOH-15 | AM-1.74 | 1.19 (I) | 827.34 |
| 159 | MC-COOH-22 | AM-1.1 | 1.05 (B) | 858.41 |
| 160 | MC-COOH-23 | AM-1.1 | 1.07 (B) | 858.42 |
| 161 | MC-COOH-2 | AM-2.21 | 1.23 (H) | 800.8 |
| 162 | MC-COOH-20 | AM-1.1 | 1.32 (H) | 848.8 |
| 163 | MC-COOH-21 | AM-1.1 | 1.33 (H) | 848.8 |
| 164 | MC-COOH-20 | AM-1.4 | 1.33 (H) | 834.8 |
| 165 | MC-COOH-21 | AM-1.4 | 1.36 (H) | 834.8 |
| 166 | MC-COOH-6 | AM-1.80 | 1.26 (H) | 851.7 |
| 167 | MC-COOH-6 | AM-1.78 | 1.12 (H) | 843.7 |
| 168 | MC-COOH-6 | AM-1.76 | 1.21 (H) | 790.7 |
| 169 | MC-COOH-6 | AM-1.77 | 1.34 (H) | 833.7 |
| 170 | MC-COOH-6 | AM-1.79 | 1.20 (H) | 850.7 |
| 171 | MC-COOH-25 | AM-1.83 | 1.27 (H) | 813.7 |
| 172 | MC-COOH-6 | AM-15.1 | 1.16 (H) | 795.6 |
| 173 | MC-COOH-6 | AM-1.81 | 1.17 (H) | 833.5 |
| 174 | MC-COOH-6 | AM-14.1 | 1.13 (H) | 827.7 |
| 175 | MC-COOH-7 | AM-1.82 | 1.26 (H) | 813.7 |
| 176 | MC-COOH-24 | AM-2.10 | 1.08 (H) | 771.8 |
| 177 | MC-COOH-24 | AM-1.84 | 1.24 (H) | 800.6 |
| 178 | MC-COOH-24 | AM-1.85 | 1.22 (H) | 801.6 |
| 179 | MC-COOH-24 | AM-15.1 | 1.14 (H) | 771.7 |
| 180 | MC-COOH-24 | AM-1.4 | 1.20 (H) | 771.7 |
| 181 | MC-COOH-24 | AM-2.12 | 1.17 (H) | 819.7 |
| 182 | MC-COOH-24 | AM-1.23 | 1.23 (H) | 809.7 |
| 183 | MC-COOH-24 | AM-1.6 | 1.22 (H) | 741.7 |
| 184 | MC-COOH-24 | AM-1.12 | 1.30 (H) | 819.6 |
| 185 | MC-COOH-24 | AM-1.5 | 1.20 (H) | 771.7 |
| 186 | MC-COOH-24 | AM-1.9 | 1.26 (H) | 755.7 |
| 187 | MC-COOH-24 | AM-2.23 | 1.11 (H) | 772.6 |
| 188 | MC-COOH-6 | AM-16.1 | 1.07 (H) | 785.6 |
| 189 | MC-COOH-24 | AM-17.1 | 1.23 (H) | 765.6 |
| 190 | MC-COOH-24 | AM-1.49 | 1.19 (H) | 788.7 |
| 191 | MC-COOH-24 | AM-5.3 | 1.16 (H) | 782.7 |
| 192 | MC-COOH-2 | AM-1.8 | 1.20 (H) | 744.7 |
| 193 | MC-COOH-2 | AM-1.5 | 1.13 (H) | 760.7 |
| 194 | MC-COOH-2 | AM-1.58 | 1.19 (H) | 774.7 |
| 195 | MC-COOH-2 | AM-1.4 | 1.14 (H) | 760.7 |
| 196 | MC-COOH-2 | AM-1.13 | 1.16 (H) | 748.6 |
| 197 | MC-COOH-2 | AM-1.60 | 0.94 (H) | 773.8 |
| 198 | MC-COOH-15 | AM-1.1 | 1.42 (H) | 840.7 |
| 199 | MC-COOH-15 | AM-1.4 | 1.44 (H) | 826.8 |
| 200 | MC-COOH-15 | AM-3.2 | 1.45 (H) | 829.7 |
| 201 | MC-COOH-15 | AM-1.58 | 1.48 (H) | 840.7 |
| 202 | MC-COOH-15 | AM-1.8 | 1.50 (H) | 810.7 |
| 203 | MC-COOH-15 | AM-1.60 | 1.24 (H) | 839.7 |
| 204 | MC-COOH-15 | AM-1.13 | 1.46 (H) | 814.7 |
| 205 | MC-COOH-15 | AM-1.7 | 1.44 (H) | 856.8 |
| 206 | MC-COOH-15 | AM-1.61 | 1.44 (H) | 802.7 |
| 207 | MC-COOH-15 | AM-1.62 | 1.18 (H) | 827.7 |
| 208 | MC-COOH-15 | AM-1.63 | 1.40 (H) | 786.6 |
| 209 | MC-COOH-15 | AM-1.66 | 1.35 (H) | 814.8 |
| 210 | MC-COOH-15 | AM-1.65 | 1.33 (H) | 764.6 |
| 211 | MC-COOH-15 | AM-1.6 | 1.45 (H) | 796.7 |
| 212 | MC-COOH-15 | AM-1.64 | 1.42 (H) | 804.7 |
| 213 | MC-COOH-15 | AM-1.67 | 1.46 (H) | 854.8 |
| 214 | MC-COOH-15 | AM-1.40 | 1.34 (H) | 801.8 |
| 215 | MC-COOH-15 | AM-1.10 | 1.53 (H) | 824.8 |
| 216 | MC-COOH-15 | AM-1.68 | 1.38 (H) | 792.8 |
| 217 | MC-COOH-15 | AM-1.69 | 1.43 (H) | 862.8 |
| 218 | MC-COOH-15 | AM-1.71 | 1.24 (H) | 841.8 |
| 219 | MC-COOH-15 | AM-1.5 | 1.44 (H) | 826.8 |
| 220 | MC-COOH-15 | AM-1.70 | 1.42 (H) | 827.7 |
| 221 | MC-COOH-5 | AM-1.84 | 1.18 (H) | 822.6 |
| 765 | MC-COOH-27 | AM-20.1 | 1.07 (H) | 754.4 |
| 766 | MC-COOH-26 | AM-20.1 | 1.19 (H) | 794.5 |
| 767 | MC-COOH-24 | AM-20.1 | 1.14 (H) | 780.4 |
| 830 | MC-COOH-6 | AM-1.94 | 0.99 (H) | 813.4 |
| 831 | MC-COOH-24 | AM-1.89 | 1.17 (H) | 809.4 |
| 832 | MC-COOH-24 | AM-1.91 | 1.08 (H) | 782.4 |
| 833 | MC-COOH-24 | AM-16.2 | 1.05 (H) | 773.4 |
| 834 | MC-COOH-26 | AM-19.3 | 1.11 (H) | 784.4 |
| 835 | MC-COOH-26 | AM-16.2 | 1.09 (H) | 787.4 |
| 836 | MC-COOH-26 | AM-19.2 | 1.08 (H) | 771.4 |
| 837 | MC-COOH-28 | AM-19.1 | 1.15 (H) | 947.4 |
| 838 | MC-COOH-30 | AM-16.2 | 1.15 (H) | 847.4 |
| 839 | MC-COOH-30 | AM-13.1 | 1.13 (H) | 836.4 |
| 840 | MC-COOH-28 | AM-1.91 | 1.16 (H) | 957.4 |
| 841 | MC-COOH-30 | AM-1.35 | 1.17 (H) | 847.4 |
| 842 | MC-COOH-28 | AM-1.88 | 1.11 (H) | 932.4 |
| 843 | MC-COOH-28 | AM-1.95 | 1.14 (H) | 947.4 |
| 844 | MC-COOH-28 | AM-1.93 | 0.98 (H) | 958.4 |
| 845 | MC-COOH-28 | AM-1.65 | 1.09 (H) | 884.4 |
| 846 | MC-COOH-28 | AM-1.92 | 1.21 (H) | 938.4 |
| 847 | MC-COOH-28 | AM-1.62 | 0.96 (H) | 947.4 |
| 848 | MC-COOH-29 | AM-1.88 | 1.18 (H) | 881.4 |
| 849 | MC-COOH-29 | AM-1.35 | 1.20 (H) | 897.4 |
| 850 | MC-COOH-24 | AM-1.45 | 1.06 (H) | 745.4 |

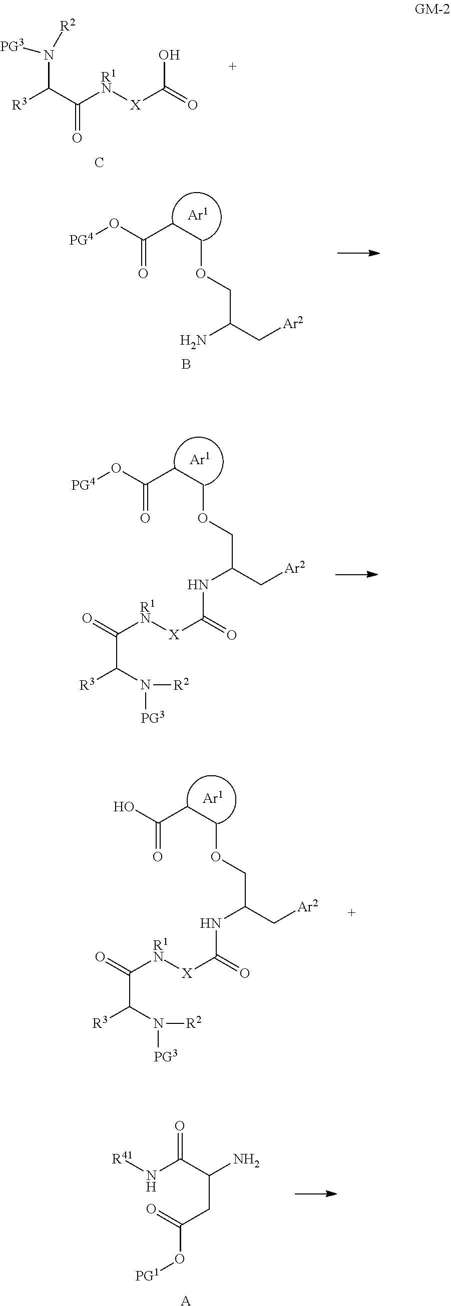

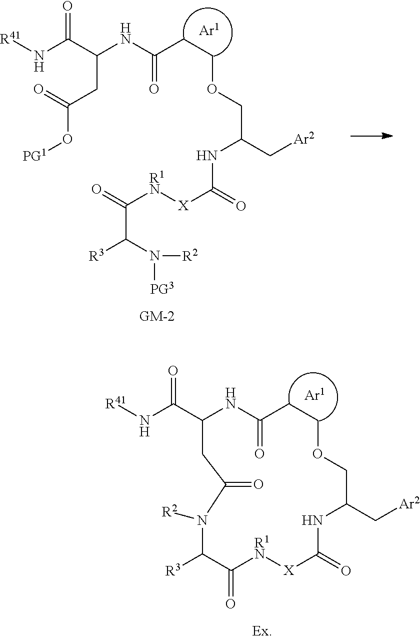

Listed in Table MC-2 below are compounds of general formula (I) that are prepared from the corresponding building blocks A, B, and C in analogy to the corresponding General Method 2 (GM-2) (See Ex. 222).

TABLE MC-2
(GM-2)

| | | | | t_{R} [min] | |
|---|---|---|---|---|---|
| | | | | LC-MS | MS-data m/z |
| Ex. | A | B | C | Method H | [M + H]⁺ |
| 223 | A-1.14 | B-1.16 | C-1.1 | 1.13 | 758.6 |
| 224 | A-1.1 | B-1.16 | C-1.1 | 1.15 | 744.7 |
| 225 | A-1.12 | B-1.16 | C-1.1 | 1.21 | 758.7 |
| 226 | A-1.6 | B-1.16 | C-1.1 | 1.11 | 772.5 |
| 227 | A-1.25 | B-1.16 | C-1.1 | 1.27 | 742.6 |
| 228 | A-1.14 | B-2.13 | C-1.1 | 1.14 | 744.5 |
| 229 | A-1.14 | B-1.3 | C-1.1 | 1.17 | 728.6 |
| 230 | A-1.1 | B-1.3 | C-1.1 | 1.20 | 714.7 |
| 231 | A-1.1 | B-2.13 | C-1.1 | 1.17 | 730.7 |
| 232 | A-1.1 | B-1.22 | C-1.1 | 1.16 | 730.7 |
| 233 | A-1.1 | B-1.4 | C-1.1 | 1.19 | 714.7 |
| 234 | A-1.14 | B-1.9 | C-1.1 | 1.16 | 732.5 |
| 235 | A-1.14 | B-1.2 | C-1.1 | 1.15 | 728.5 |
| 236 | A-1.14 | B-1.12 | C-1.1 | 1.22 | 764.7 |
| 237 | A-1.1 | B-1.8 | C-1.1 | 1.22 | 732.7 [M − H]⁻ |
| 238 | A-1.1 | B-1.12 | C-1.1 | 1.25 | 750.6 |
| 239 | A-1.25 | B-1.5 | C-1.1 | 1.24 | 728.6 |
| 240 | A-1.1 | B-2.4 | C-1.1 | 1.13 | 706.6 |
| 241 | A-1.1 | B-2.3 | C-1.1 | 1.13 | 721.6 |
| 242 | A-1.14 | B-5.1 | C-10.19 | 1.42 | 843.8 |
| 243 | A-1.1 | B-5.1 | C-10.19 | 1.45 | 829.7 |
| 244 | A-1.23 | B-5.1 | C-10.19 | 1.44 | 832.9 |
| 245 | A-1.2 | B-2.13 | C-1.1 | 1.21 | 756.7 |
| 246 | A-1.14 | B-1.2 | C-10.19 | 1.46 | 824.7 |
| 247 | A-1.1 | B-1.2 | C-10.19 | 1.49 | 810.7 |
| 248 | A-1.14 | B-2.13 | C-10.19 | 1.44 | 840.8 |
| 249 | A-1.1 | B-2.13 | C-10.19 | 1.47 | 826.8 |
| 250 | A-1.14 | B-1.12 | C-10.19 | 1.51 | 860.7 |
| 251 | A-1.1 | B-1.12 | C-10.19 | 1.53 | 846.7 |
| 252 | A-1.25 | B-3.1 | C-1.1 | 1.04 | 699.5 |
| 253 | A-1.14 | B-1.8 | C-10.19 | 1.49 | 844.6 |
| 254 | A-1.1 | B-1.8 | C-10.19 | 1.51 | 828.8 [M − H]⁻ |
| 255 | A-1.14 | B-1.1 | C-10.19 | 1.42 | 870.7 |
| 256 | A-1.1 | B-1.1 | C-10.19 | 1.44 | 856.9 |
| 257 | A-1.14 | B-1.5 | C-10.23 | 1.24 | 784.6 |
| 258 | A-1.1 | B-1.5 | C-10.23 | 1.26 | 770.7 |
| 259 | A-1.4 | B-1.5 | C-2.1 | 1.22 | 820.5 |
| 260 | A-1.5 | B-1.5 | C-2.1 | 1.22 | 812.7 |
| 261 | A-1.2 | B-3.1 | C-10.19 | 1.31 | 823.7 |
| 262 | A-1.3 | B-3.1 | C-10.19 | 1.26 | 809.7 |
| 263 | A-1.14 | B-1.5 | C-10.24 | 1.28 | 798.8 |
| 264 | A-1.1 | B-1.5 | C-10.24 | 1.29 | 784.8 |
| 265 | A-1.14 | B-3.2 | C-10.19 | 1.36 | 811.7 |
| 266 | A-1.1 | B-3.2 | C-10.19 | 1.39 | 797.7 |
| 267 | A-1.14 | B-1.13 | C-10.19 | 1.49 | 860.7 |
| 268 | A-1.1 | B-1.13 | C-10.19 | 1.51 | 846.7 |
| 269 | A-1.14 | B-1.13 | C-1.1 | 1.20 | 764.7 |
| 270 | A-1.14 | B-1.10 | C-10.19 | 1.16 | 825.8 |
| 271 | A-1.1 | B-1.10 | C-10.19 | 1.18 | 811.7 |
| 272 | A-1.14 | B-1.1 | C-10.24 | 1.27 | 828.7 |
| 273 | A-1.1 | B-1.1 | C-10.24 | 1.30 | 814.6 |
| 274 | A-1.14 | B-1.5 | C-10.26 | 1.38 | 838.7 |
| 275 | A-1.2 | B-1.1 | C-10.19 | 1.47 | 882.9 |
| 276 | A-1.3 | B-1.1 | C-10.19 | 1.44 | 868.8 |
| 277 | A-1.1 | B-2.1 | C-1.1 | 1.14 | 718.6 |
| 278 | A-1.14 | B-2.1 | C-1.1 | 1.11 | 732.6 |
| 279 | A-1.14 | B-2.2 | C-1.1 | 1.15 | 746.7 [M − H]⁻ |
| 280 | A-1.1 | B-1.17 | C-2.2 | 1.30 | 784.6 |
| 281 | A-1.14 | B-1.18 | C-10.19 | 1.37 | 841.7 |
| 282 | A-1.1 | B-1.18 | C-10.19 | 1.39 | 827.8 |
| 283 | A-1.6 | B-1.17 | C-2.2 | 1.27 | 812.8 |
| 284 | A-1.14 | B-1.13 | C-10.2 | 1.37 | 874.8 |
| 285 | A-1.14 | B-1.13 | C-10.4 | 1.42 | 888.8 |
| 286 | A-1.14 | B-1.13 | C-10.1 | 1.39 | 874.8 |
| 287 | A-1.14 | B-1.13 | C-10.3 | 1.44 | 888.8 |
| 288 | A-1.14 | B-1.13 | C-10.14 | 1.38 | 888.8 |
| 289 | A-1.14 | B-1.13 | C-10.15 | 1.43 | 902.8 |
| 290 | A-1.14 | B-1.13 | C-10.16 | 1.54 | 886.8 |
| 291 | A-1.14 | B-1.13 | C-10.17 | 1.58 | 900.9 |
| 292 | A-1.14 | B-1.13 | C-10.18 | 1.50 | 872.7 |
| 293 | A-1.1 | B-1.13 | C-10.2 | 1.40 | 860.7 |
| 294 | A-1.1 | B-1.13 | C-10.4 | 1.45 | 874.7 |
| 295 | A-1.1 | B-1.13 | C-10.1 | 1.42 | 860.7 |
| 296 | A-1.1 | B-1.13 | C-10.3 | 1.47 | 874.7 |
| 297 | A-1.1 | B-1.13 | C-4.10 | 1.35 | 848.7 |
| 298 | A-1.1 | B-1.13 | C-10.14 | 1.41 | 874.8 |
| 299 | A-1.1 | B-1.13 | C-10.15 | 1.46 | 888.9 |
| 300 | A-1.1 | B-1.13 | C-10.16 | 1.57 | 872.8 |
| 301 | A-1.1 | B-1.13 | C-10.17 | 1.60 | 886.9 |
| 302 | A-1.1 | B-1.13 | C-10.18 | 1.53 | 858.7 |
| 303 | A-1.1 | B-1.13 | C-10.19 | 1.51 | 846.8 |
| 304 | A-1.14 | B-1.5 | C-4.10 | 1.24 | 842.7 |
| 305 | A-1.14 | B-1.5 | C-10.20 | 1.26 | 828.7 |
| 306 | A-1.14 | B-1.5 | C-10.21 | 1.24 | 828.6 |
| 307 | A-1.14 | B-1.5 | C-10.1 | 1.32 | 854.8 |
| 308 | A-1.14 | B-1.5 | C-10.2 | 1.30 | 854.8 |
| 309 | A-1.14 | B-1.5 | C-10.4 | 1.35 | 868.7 |
| 310 | A-1.1 | B-1.5 | C-4.10 | 1.27 | 828.7 |
| 311 | A-1.1 | B-1.5 | C-10.20 | 1.28 | 814.7 |
| 312 | A-1.1 | B-1.5 | C-10.21 | 1.26 | 814.7 |
| 313 | A-1.1 | B-1.5 | C-10.1 | 1.35 | 840.7 |
| 314 | A-1.1 | B-1.5 | C-10.4 | 1.38 | 854.7 |
| 315 | A-1.1 | B-1.14 | C-10.19 | 1.43 | 847.8 |
| 316 | A-1.23 | B-1.14 | C-10.19 | 1.43 | 850.8 |
| 317 | A-1.1 | B-4.1 | C-1.1 | 1.23 | 784.6 |
| 318 | A-1.14 | B-1.14 | C-1.1 | 1.10 | 765.5 |
| 319 | A-1.1 | B-1.14 | C-1.1 | 1.12 | 751.6 |
| 320 | A-1.23 | B-1.14 | C-1.1 | 1.12 | 754.6 |
| 321 | A-1.14 | B-4.2 | C-1.1 | 1.09 | 758.6 |
| 322 | A-1.14 | B-4.3 | C-1.1 | 1.09 | 760.5 |
| 323 | A-1.1 | B-4.2 | C-1.1 | 1.12 | 744.6 |
| 324 | A-1.1 | B-4.3 | C-1.1 | 1.11 | 746.6 |
| 325 | A-1.15 | B-1.13 | C-2.1 | 1.25 | 822.7 |
| 326 | A-1.14 | B-1.13 | C-2.1 | 1.23 | 806.7 |
| 327 | A-1.16 | B-1.13 | C-2.1 | 1.21 | 803.7 |
| 328 | A-1.15 | B-2.7 | C-2.2 | 1.04 | 824.8 |
| 329 | A-1.14 | B-2.7 | C-2.2 | 1.01 | 808.6 |
| 330 | A-1.14 | B-1.13 | C-4.4 | 1.26 | 874.7 |
| 331 | A-1.14 | B-1.13 | C-4.5 | 1.38 | 888.8 |
| 332 | A-1.14 | B-1.13 | C-4.6 | 1.27 | 848.8 |
| 333 | A-1.14 | B-1.13 | C-4.7 | 1.27 | 862.7 |
| 334 | A-1.14 | B-1.13 | C-4.8 | 1.29 | 932.8 |
| 335 | A-1.14 | B-1.13 | C-4.2 | 1.35 | 850.7 |
| 336 | A-1.14 | B-1.13 | C-4.11 | 1.29 | 862.7 |
| 337 | A-1.14 | B-1.13 | C-10.5 | 1.33 | 860.6 |
| 338 | A-1.14 | B-1.13 | C-10.6 | 1.35 | 860.7 |
| 339 | A-1.14 | B-1.13 | C-4.12 | 1.38 | 842.8 |
| 340 | A-1.14 | B-1.13 | C-10.7 | 1.29 | 846.8 |
| 341 | A-1.14 | B-1.13 | C-10.26 | 1.45 | 858.8 |
| 342 | A-1.1 | B-2.6 | C-2.8 | 1.19 | 781.6 |
| 343 | A-1.1 | B-2.6 | C-2.2 | 1.22 | 781.7 |
| 344 | A-1.8 | B-1.14 | C-10.19 | 1.32 | 822.7 |
| 345 | A-1.14 | B-4.2 | C-10.19 | 1.41 | 854.7 |
| 346 | A-1.1 | B-4.2 | C-10.19 | 1.43 | 840.7 |
| 347 | A-1.23 | B-4.2 | C-10.19 | 1.43 | 843.7 |
| 348 | A-1.1 | B-2.7 | C-3.7 | 0.96 | 835.8 |
| 349 | A-1.14 | B-1.20 | C-1.1 | 1.12 | 783.5 |
| 350 | A-1.1 | B-1.20 | C-1.1 | 1.14 | 769.6 |
| 351 | A-1.1 | B-2.15 | C-2.2 | 1.22 | 795.6 |
| 352 | A-1.1 | B-2.5 | C-3.3 | 0.78 | 838.8 |
| 353 | A-1.1 | B-2.7 | C-3.3 | 0.68 | 837.7 |
| 354 | A-1.1 | B-1.13 | C-6.3 | 0.97 | 849.7 |
| 355 | A-1.1 | B-1.13 | C-3.5 | 0.97 | 845.7 |
| 356 | A-1.1 | B-1.13 | C-3.7 | 1.246 | 831.8 |
| 357* | A-1.1 | B-1.13 | C-3.7 | 1.17 | 831.7 |
| 358 | A-1.14 | B-1.13 | C-10.8 | 1.29 | 860.8 |
| 359 | A-1.14 | B-1.13 | C-10.9 | 1.28 | 860.7 |
| 360 | A-1.14 | B-1.13 | C-10.10 | 1.34 | 874.8 |
| 361 | A-1.14 | B-1.13 | C-10.11 | 1.34 | 874.8 |
| 362 | A-1.14 | B-1.13 | C-10.12 | 1.33 | 862.8 |
| 363 | A-1.14 | B-1.13 | C-10.13 | 1.33 | 862.8 |
| 364 | A-1.1 | B-2.7 | C-3.4 | 0.82 | 851.7 |
| 365 | A-1.1 | B-1.14 | C-2.2 | 1.27 | 791.8 |
| 366 | A-1.1 | B-1.14 | C-2.8 | 1.24 | 791.7 |
| 367 | A-1.8 | B-1.14 | C-2.2 | 1.15 | 766.6 |
| 368 | A-1.15 | B-2.15 | C-2.2 | 1.22 | 825.7 |
| 369 | A-1.1 | B-1.13 | C-7.1 | 1.44 | 965.7 |
| 370 | A-1.11 | B-1.13 | C-2.1 | 1.23 | 809.6 |
| 371 | A-1.10 | B-1.13 | C-2.1 | 1.29 | 828.7 |
| 372 | A-1.1 | B-1.14 | C-3.4 | 0.97 | 848.7 |
| 373* | A-1.1 | B-1.14 | C-3.4 | 0.95 | 848.7 |
| 374 | A-1.1 | B-1.14 | C-3.3 | 0.84 | 834.6 |
| 375 | A-1.8 | B-1.14 | C-3.3 | 0.74 | 809.7 |
| 376 | A-1.1 | B-1.13 | C-1.3 | 1.34 | 788.6 |
| 377 | A-1.15 | B-2.9 | C-2.2 | 0.94 | 838.8 |
| 378 | A-1.9 | B-1.13 | C-2.1 | 1.16 Method I | 842.52 |
| 379 | A-1.14 | B-2.5 | C-10.19 | 1.38 | 865.8 |
| 380 | A-1.1 | B-2.5 | C-10.19 | 1.41 | 851.7 |
| 381 | A-1.23 | B-2.5 | C-10.19 | 1.41 | 854.7 |
| 382 | A-1.14 | B-1.20 | C-10.19 | 1.42 | 879.8 |
| 383 | A-1.1 | B-1.20 | C-10.19 | 1.44 | 865.7 |
| 384 | A-1.23 | B-1.20 | C-10.19 | 1.44 | 868.8 |
| 385 | A-1.1 | B-1.13 | C-1.2 | 1.30 | 776.6 |
| 386 | A-1.1 | B-1.13 | C-2.7 | 1.31 | 762.6 |
| 387 | A-1.7 | B-1.13 | C-2.1 | 1.26 | 829.8 |
| 388* | A-1.1 | B-1.14 | C-3.7 | 1.14 | 832.7 |
| 389 | A-1.1 | B-1.14 | C-3.7 | 1.13 | 832.7 |
| 390* | A-1.1 | B-1.14 | C-3.5 | 0.93 | 846.8 |
| 391 | A-1.1 | B-1.14 | C-3.5 | 0.89 | 846.7 |
| 392* | A-1.14 | B-1.14 | C-3.7 | 1.13 | 846.8 |
| 393 | A-1.14 | B-1.14 | C-3.7 | 1.13 | 846.8 |
| 394* | A-1.14 | B-1.14 | C-3.5 | 0.91 | 860.8 |
| 395 | A-1.14 | B-1.14 | C-3.5 | 0.88 | 860.8 |
| 396 | A-1.1 | B-1.13 | C-6.4 | 1.34 | 834.8 |
| 397 | A-1.12 | B-1.13 | C-2.1 | 1.31 | 806.8 |
| 398 | A-1.10 | B-1.14 | C-3.7 | 1.20 | 868.8 |
| 399* | A-1.10 | B-1.14 | C-3.7 | 1.18 | 868.7 |
| 400 | A-1.12 | B-1.14 | C-3.7 | 1.22 | 846.8 |
| 401* | A-1.12 | B-1.14 | C-3.7 | 1.20 | 846.8 |
| 402 | A-1.8 | B-1.14 | C-3.7 | 1.01 | 805.8 [M − H]⁻ |
| 403* | A-1.8 | B-1.14 | C-3.7 | 1.03 | 807.7 |
| 404 | A-1.11 | B-1.14 | C-3.7 | 1.14 | 849.8 |
| 405* | A-1.11 | B-1.14 | C-3.7 | 1.16 | 849.8 |
| 406 | A-1.1 | B-2.8 | C-2.2 | 1.27 | 795.8 |
| 407 | A-1.1 | B-2.8 | C-2.1 | 1.13 | 797.6 |
| 408 | A-1.14 | B-4.4 | C-1.1 | 1.20 | 794.6 |
| 409 | A-1.1 | B-4.4 | C-1.1 | 1.22 | 780.6 |
| 410 | A-1.8 | B-2.5 | C-10.19 | 1.230 | 826.8 |
| 411 | A-1.8 | B-1.20 | C-10.19 | 1.34 | 840.8 |
| 412 | A-1.1 | B-1.14 | C-2.1 | 1.16 | 793.6 |
| 413* | A-1.1 | B-1.14 | C-2.1 | 1.16 | 793.6 |
| 414 | A-1.10 | B-1.14 | C-6.3 | 0.94 | 886.7 |
| 415 | A-1.11 | B-1.14 | C-6.3 | 0.87 | 867.7 |
| 416 | A-1.1 | B-1.13 | C-6.2 | 0.90 | 835.6 |
| 417 | A-1.1 | B-2.5 | C-3.7 | 1.09 | 836.6 |
| 418 | A-1.12 | B-2.5 | C-3.7 | 1.15 | 850.7 |
| 419 | A-1.10 | B-2.5 | C-3.7 | 1.14 | 872.7 |
| 420 | A-1.11 | B-2.5 | C-3.7 | 1.07 | 853.6 |
| 421 | A-1.6 | B-2.5 | C-3.7 | 1.07 | 864.8 |
| 422 | A-1.26 | B-2.5 | C-10.19 | 1.46 | 885.8 |
| 423 | A-1.1 | B-6.2 | C-2.2 | 1.25 | 794.6 |
| 424 | A-1.1 | B-6.2 | C-2.1 | 1.13 | 796.7 |
| 425 | A-1.1 | B-2.5 | C-3.6 | 0.81 | 850.7 |
| 426 | A-1.10 | B-2.5 | C-3.6 | 0.86 | 886.7 |
| 427 | A-1.12 | B-2.5 | C-3.6 | 0.85 | 864.7 |
| 428 | A-1.7 | B-2.5 | C-3.6 | 0.82 | 887.8 |
| 429 | A-1.8 | B-6.2 | C-2.2 | 1.13 | 769.6 |
| 430 | A-1.14 | B-2.10 | C-1.1 | 1.11 | 754.7 |
| 431 | A-1.1 | B-1.13 | C-12.1 | 1.35 | 822.7 |
| 432 | A-1.8 | B-1.14 | C-11.1 | 1.16 | 778.7 |
| 433 | A-1.1 | B-1.14 | C-1.2 | 1.21 | 777.6 |
| 434 | A-1.8 | B-1.14 | C-1.2 | 1.08 | 752.7 |
| 435 | A-1.8 | B-2.5 | C-2.6 | 1.06 | 798.7 |
| 436 | A-1.8 | B-2.5 | C-2.5 | 1.19 | 796.6 |
| 437* | A-1.8 | B-2.5 | C-2.5 | 1.16 | 796.8 |
| 438 | A-1.1 | B-1.18 | C-1.2 | 1.13 | 757.6 |
| 439 | A-1.1 | B-1.20 | C-1.2 | 1.23 | 795.7 |
| 440 | A-1.8 | B-1.20 | C-1.2 | 1.09 | 770.6 |
| 441 | A-1.1 | B-4.7 | C-1.1 | 1.01 | 751.7 |
| 442 | A-1.1 | B-2.10 | C-1.2 | 1.19 | 766.6 |
| 443 | A-1.8 | B-1.20 | C-2.2 | 1.17 | 784.7 |
| 444 | A-1.8 | B-4.5 | C-2.2 | 0.83 | 730.6 |
| 445 | A-1.8 | B-1.14 | C-10.20 | 1.13 | 810.6 |
| 446 | A-1.8 | B-1.14 | C-10.12 | 1.13 | 824.7 |
| 447 | A-1.1 | B-4.6 | C-1.2 | 1.18 | 768.6 |
| 448 | A-1.8 | B-2.10 | C-1.2 | 1.06 | 741.6 |
| 449 | A-1.1 | B-2.10 | C-1.1 | 1.12 | 740.5 |
| 450 | A-1.1 | B-2.11 | C-1.1 | 1.17 | 742.6 |
| 451 | A-1.1 | B-1.13 | C-6.1 | 0.89 | 849.7 |
| 452 | A-1.8 | B-1.13 | C-6.1 | 0.78 | 824.7 |
| 453 | A-1.17 | B-1.14 | C-1.2 | 1.21 | 814.8 |
| 454 | A-1.7 | B-1.14 | C-1.2 | 1.21 | 814.6 |
| 455 | A-1.8 | B-4.6 | C-2.2 | 1.13 | 757.6 |
| 456 | A-1.1 | B-4.6 | C-2.2 | 1.25 | 782.6 |
| 457 | A-1.1 | B-1.13 | C-13.1 | 1.33 | 820.7 |
| 458 | A-1.8 | B-1.14 | C-7.1 | 1.25 | 941.6 |
| 459 | A-1.1 | B-1.14 | C-3.1 | 0.82 | 806.8 |
| 460 | A-1.1 | B-4.7 | C-1.2 | 1.09 | 777.7 |
| 461 | A-1.17 | B-1.14 | C-2.2 | 1.27 | 828.7 |
| 462 | A-1.17 | B-1.14 | C-2.8 | 1.24 | 828.7 |
| 463 | A-1.7 | B-1.14 | C-2.2 | 1.28 | 828.8 |
| 464 | A-1.7 | B-1.14 | C-2.8 | 1.24 | 828.6 |
| 465 | A-1.1 | B-7.1 | C-1.1 | 0.82 | 751.6 |
| 466 | A-1.17 | B-1.18 | C-2.2 | 1.21 | 808.7 |
| 467 | A-1.17 | B-1.18 | C-2.8 | 1.17 | 808.6 |
| 468 | A-1.7 | B-1.18 | C-2.2 | 1.21 | 808.7 |
| 469 | A-1.1 | B-2.10 | C-2.2 | 1.25 | 780.6 |
| 470 | A-1.8 | B-2.10 | C-2.2 | 1.12 | 755.6 |
| 471 | A-1.1 | B-4.8 | C-1.2 | 0.89 | 777.7 |
| 472 | A-1.7 | B-4.7 | C-2.2 | 1.16 | 828.8 |
| 473 | A-1.7 | B-4.7 | C-2.8 | 1.13 | 828.6 |
| 474 | A-1.8 | B-4.7 | C-2.2 | 1.02 | 766.7 |
| 475 | A-1.8 | B-1.13 | C-9.1 | 1.15 | 907.7 |
| 476 | A-1.24 | B-1.14 | C-1.2 | 1.14 | 778.7 |
| 477 | A-1.8 | B-1.18 | C-2.2 | 1.09 | 746.7 |
| 478 | A-1.20 | B-1.18 | C-2.2 | 1.24 | 777.7 |
| 479 | A-1.8 | B-1.13 | C-9.2 | 1.19 | 937.7 |
| 480 | A-1.8 | B-1.19 | C-2.2 | 1.19 | 784.7 |
| 481 | A-1.7 | B-1.19 | C-2.2 | 1.32 | 846.7 |
| 482 | A-1.20 | B-2.5 | C-2.2 | 1.26 | 801.6 |
| 483 | A-1.19 | B-1.18 | C-2.2 | 1.12 | 773.6 |
| 484 | A-1.20 | B-2.10 | C-2.2 | 1.28 | 786.7 |
| 485 | A-1.20 | B-2.7 | C-2.2 | 1.06 | 800.6 |
| 486 | A-1.19 | B-4.7 | C-2.2 | 1.07 | 793.8 |
| 487 | A-1.8 | B-1.23 | C-2.2 | 1.12 | 766.7 |
| 488 | A-1.1 | B-2.12 | C-1.2 | 1.15 | 757.6 |
| 489 | A-1.19 | B-1.19 | C-2.1 | 1.12 | 813.7 |
| 490 | A-1.20 | B-6.2 | C-2.2 | 1.28 | 800.6 |
| 491 | A-1.20 | B-4.6 | C-2.2 | 1.28 | 788.6 |
| 492 | A-1.7 | B-1.13 | C-2.4 | 1.32 | 857.8 |
| 493 | A-1.7 | B-1.14 | C-2.4 | 1.23 | 858.7 |
| 494 | A-1.1 | B-7.1 | C-1.2 | 0.91 | 777.7 |
| 495 | A-1.8 | B-1.14 | C-5.1 | 1.24 | 828.6 |
| 496 | A-1.21 | B-1.14 | C-1.2 | 1.18 | 825.8 |
| 497 | A-1.18 | B-1.20 | C-2.8 | 1.20 | 810.6 |
| 498 | A-1.19 | B-1.20 | C-2.2 | 1.20 | 811.6 |
| 499 | A-1.24 | B-1.20 | C-2.2 | 1.22 | 810.6 |
| 500 | A-1.24 | B-1.20 | C-2.8 | 1.18 | 810.6 |
| 501 | A-1.22 | B-1.20 | C-2.2 | 1.22 | 811.7 |
| 502 | A-1.27 | B-1.14 | C-1.2 | 1.15 | 825.7 |
| 503 | A-1.1 | B-1.14 | C-5.2 | 1.21 | 779.7 |
| 504 | A-1.18 | B-4.7 | C-2.2 | 1.11 | 792.8 |
| 505 | A-1.22 | B-4.7 | C-2.2 | 1.07 | 793.6 |
| 506 | A-1.8 | B-1.14 | C-14.1 | 1.20 | 780.8 |
| 507 | A-1.1 | B-4.9 | C-1.1 | 1.14 | 769.5 |
| 508 | A-1.8 | B-1.13 | C-5.3 | 1.22 | 765.7 |
| 509 | A-1.1 | B-1.14 | C-8.1 | 1.36 | 868.7 |
| 510 | A-1.8 | B-1.14 | C-8.1 | 1.26 | 843.8 |
| 511 | A-1.22 | B-1.18 | C-2.2 | 1.14 | 773.7 |
| 512 | A-1.18 | B-1.18 | C-2.2 | 1.16 | 772.7 |
| 513 | A-1.1 | B-4.10 | C-1.1 | 1.07 | 731.6 |
| 514 | A-1.24 | B-4.7 | C-2.2 | 1.09 | 792.7 |
| 515 | A-1.22 | B-4.11 | C-2.2 | 1.21 | 773.7 |
| 516 | A-1.19 | B-4.11 | C-2.2 | 1.19 | 773.7 |
| 517 | A-1.24 | B-2.12 | C-2.2 | 1.16 | 772.7 |
| 518 | A-1.18 | B-2.12 | C-2.2 | 1.18 | 772.6 |
| 519 | A-1.24 | B-1.19 | C-1.2 | 1.18 | 796.6 |
| 520 | A-1.19 | B-1.19 | C-2.8 | 1.18 | 811.7 |
| 521 | A-1.22 | B-1.19 | C-2.8 | 1.20 | 811.7 |
| 522 | A-1.7 | B-1.20 | C-2.2 | 1.29 | 846.7 |
| 523 | A-1.24 | B-1.18 | C-2.2 | 1.15 | 772.7 |
| 524 | A-1.24 | B-1.19 | C-8.1 | 1.34 | 887.7 |
| 525 | A-1.22 | B-1.19 | C-8.1 | 1.33 | 888.7 |
| 526 | A-1.18 | B-4.11 | C-2.2 | 1.22 | 772.7 |
| 527 | A-1.8 | B-1.26 | C-2.2 | 1.23 | 800.6 |
| 528 | A-1.1 | B-1.26 | C-2.2 | 1.36 | 823.9 [M − H]⁻ |
| 529 | A-1.22 | B-2.12 | C-2.2 | 1.16 | 773.7 |
| 530 | A-1.1 | B-2.16 | C-1.1 | 1.20 | 751.6 |
| 531 | A-1.1 | B-5.3 | C-1.1 | 1.19 | 754.7 |
| 532 | A-1.24 | B-5.3 | C-1.1 | 1.11 | 755.6 |
| 533 | A-1.28 | B-4.12 | C-2.2 | 1.01 Method I | 776.7 |
| 534 | A-1.8 | B-1.27 | C-2.2 | 1.14 | 784.6 |
| 535 | A-1.1 | B-1.27 | C-2.2 | 1.07 Method B | 809.26 |
| 536 | A-1.1 | B-2.5 | C-8.1 | 1.32 | 872.7 |
| 537 | A-1.1 | B-1.19 | C-2.3 | 1.15 | 795.6 |
| 538 | A-1.1 | B-1.18 | C-8.1 | 1.30 | 848.8 |
| 539 | A-1.19 | B-1.19 | C-18.1 | 1.34 | 903.8 |
| 540 | A-1.19 | B-1.19 | C-18.1 | 1.30 | 903.8 |
| 541 | A-1.19 | B-4.9 | C-2.2 | 1.21 | 811.6 |
| 542 | A-1.8 | B-1.18 | C-8.1 | 1.20 | 823.7 |
| 543 | A-1.24 | B-4.7 | C-1.2 | 1.01 | 778.6 |
| 544 | A-1.19 | B-4.7 | C-1.2 | 0.99 | 779.7 |
| 545 | A-1.24 | B-4.9 | C-1.2 | 1.16 | 796.6 |
| 546 | A-1.19 | B-4.9 | C-1.2 | 1.14 | 797.6 |
| 547 | A-1.8 | B-1.14 | C-18.3 | 1.19 | 768.6 |
| 548 | A-1.8 | B-1.14 | C-18.4 | 1.25 | 782.6 |
| 549* | A-1.1 | B-1.18 | C-8.1 | 1.30 | 848.8 |
| 550* | A-1.8 | B-1.18 | C-8.1 | 1.19 | 823.8 |
| 551 | A-1.1 | B-5.3 | C-1.2 | 1.25 | 780.8 |
| 552 | A-1.24 | B-5.3 | C-1.2 | 1.19 | 781.7 |
| 553 | A-1.19 | B-5.3 | C-1.2 | 1.17 | 782.7 |
| 554 | A-1.22 | B-5.3 | C-1.2 | 1.17 | 782.6 |
| 555 | A-1.1 | B-2.16 | C-1.2 | 1.28 | 777.6 |
| 556 | A-1.24 | B-2.16 | C-1.2 | 1.21 | 778.7 |
| 557 | A-1.19 | B-1.18 | C-18.1 | 1.20 | 865.8 |
| 558 | A-1.19 | B-1.18 | C-18.1 | 1.24 | 865.7 |
| 559 | A-1.31 | B-1.14 | C-2.2 | 1.29 | 820.6 |
| 560 | A-1.31 | B-1.19 | C-2.2 | 1.33 | 838.6 |
| 561 | A-1.19 | B-5.3 | C-1.1 | 1.09 | 756.6 |
| 562 | A-1.22 | B-5.3 | C-1.1 | 1.10 | 756.6 |
| 563 | A-1.19 | B-1.27 | C-2.2 | 1.17 | 811.6 |
| 564 | A-1.8 | B-1.14 | C-2.14 | 1.26 | 846.6 |
| 565 | A-1.18 | B-4.12 | C-2.2 | 1.21 | 786.6 |
| 566 | A-1.8 | B-1.14 | C-21.1 | 1.09 | 784.6 |
| 567 | A-1.19 | B-4.12 | C-2.2 | 1.17 | 787.6 |
| 568 | A-1.31 | B-4.12 | C-2.2 | 1.28 | 814.7 |
| 569 | A-1.1 | B-1.18 | C-2.10 | 1.24 | 819.6 |
| 570 | A-1.8 | B-1.18 | C-3.4 | 0.80 | 803.7 |
| 571 | A-1.19 | B-1.18 | C-20.1 | 1.26 | 905.8 |
| 572* | A-1.1 | B-1.14 | C-1.4 | 1.19 | 765.7 |
| 573 | A-1.8 | B-1.14 | C-1.4 | 1.09 | 740.7 |
| 574 | A-1.24 | B-1.14 | C-1.4 | 1.15 | 766.7 |
| 575 | A-1.1 | B-5.3 | C-1.4 | 1.25 | 768.7 |
| 576 | A-1.8 | B-5.3 | C-1.4 | 1.12 | 743.7 |
| 577 | A-1.19 | B-1.18 | C-20.1 | 1.22 | 905.7 |
| 578 | A-1.8 | B-1.14 | C-18.2 | 1.27 | 860.7 |
| 579* | A-1.8 | B-1.14 | C-18.2 | 1.23 | 860.7 |
| 580 | A-1.28 | B-4.12 | C-2.13 | 1.24 | 802.6 |
| 581 | A-1.28 | B-4.13 | C-2.2 | 1.15 | 779.6 |
| 582 | A-1.28 | B-1.18 | C-10.19 | 1.30 | 818.7 |
| 583 | A-1.14 | B-1.4 | C-1.1 | 1.16 | 728.6 |
| 584 | A-1.1 | B-2.13 | C-2.3 | 1.16 | 756.7 |
| 585 | A-1.14 | B-1.11 | C-10.19 | 1.41 | 870.7 |
| 586 | A-1.1 | B-1.11 | C-10.19 | 1.44 | 856.7 |
| 587 | A-1.1 | B-2.13 | C-2.2 | 1.30 | 770.6 |
| 588 | A-1.1 | B-2.13 | C-2.1 | 1.17 | 772.6 |
| 589 | A-1.14 | B-3.1 | C-10.19 | 1.24 | 811.6 |
| 590 | A-1.1 | B-3.1 | C-10.19 | 1.26 | 797.7 |
| 591 | A-1.1 | B-1.5 | C-2.2 | 1.25 | 770.6 |
| 592 | A-1.3 | B-3.1 | C-2.8 | 1.04 | 753.7 |
| 593 | A-1.3 | B-3.1 | C-2.2 | 1.05 | 753.7 |
| 594 | A-1.6 | B-3.1 | C-2.2 | 1.03 | 769.7 |
| 595 | A-1.1 | B-1.13 | C-1.1 | 1.22 | 750.6 |
| 596 | A-1.2 | B-1.11 | C-10.19 | 1.48 | 882.8 |
| 597 | A-1.3 | B-1.11 | C-10.19 | 1.43 | 868.8 |
| 598 | A-1.1 | B-2.5 | C-1.1 | 1.08 | 755.5 |
| 599 | A-1.2 | B-1.13 | C-10.19 | 1.54 | 872.9 |
| 600 | A-1.3 | B-1.13 | C-10.19 | 1.51 | 856.8 [M − H]⁻ |
| 601 | A-1.1 | B-1.1 | C-2.8 | 1.24 | 800.8 |
| 602 | A-1.1 | B-1.1 | C-2.2 | 1.26 | 800.7 |
| 603 | A-1.1 | B-1.13 | C-2.2 | 1.37 | 790.8 |
| 604 | A-1.1 | B-1.13 | C-2.8 | 1.34 | 790.8 |
| 605 | A-1.1 | B-1.5 | C-3.2 | 0.79 | 799.8 |
| 606 | A-1.8 | B-1.13 | C-2.8 | 1.22 | 765.7 |
| 607 | A-1.8 | B-1.13 | C-2.2 | 1.24 | 765.6 |
| 608 | A-1.8 | B-1.13 | C-2.1 | 1.13 | 767.6 |
| 609 | A-1.8 | B-1.1 | C-2.2 | 1.14 | 775.6 |
| 610 | A-1.14 | B-1.14 | C-10.19 | 1.41 | 861.7 |
| 611 | A-1.1 | B-1.5 | C-3.3 | 0.81 | 813.8 |
| 612 | A-1.1 | B-1.5 | C-3.4 | 0.95 | 827.7 |
| 613 | A-1.15 | B-1.13 | C-3.4 | 1.05 | 877.8 |
| 614 | A-1.1 | B-2.5 | C-2.2 | 1.23 | 795.6 |
| 615 | A-1.1 | B-2.5 | C-2.8 | 1.20 | 795.8 |
| 616 | A-1.14 | B-5.2 | C-1.1 | 1.15 | 780.5 |
| 617 | A-1.8 | B-1.13 | C-3.4 | 0.94 | 822.7 |
| 618 | A-1.15 | B-1.13 | C-3.3 | 0.92 | 863.8 |
| 619 | A-1.1 | B-2.5 | C-3.4 | 0.95 | 852.8 |
| 620 | A-1.8 | B-2.5 | C-3.4 | 0.84 | 827.8 |
| 621* | A-1.8 | B-2.5 | C-3.4 | 0.83 | 827.8 |
| 622 | A-1.8 | B-1.14 | C-3.4 | 0.86 | 823.7 |
| 623 | A-1.8 | B-1.13 | C-3.3 | 0.82 | 808.7 |
| 624 | A-1.1 | B-6.1 | C-2.1 | 1.12 | 797.6 |
| 625 | A-1.10 | B-2.5 | C-2.1 | 1.15 | 833.7 |
| 626 | A-1.14 | B-1.13 | C-10.25 | 1.39 | 868.8 |
| 627 | A-1.6 | B-1.20 | C-2.1 | 1.15 | 839.7 |
| 628 | A-1.1 | B-2.5 | C-1.2 | 1.17 | 781.7 |
| 629 | A-1.8 | B-6.1 | C-2.2 | 1.14 | 770.7 |
| 630 | A-1.8 | B-6.1 | C-2.5 | 1.21 | 796.6 |
| 631* | A-1.8 | B-6.1 | C-2.5 | 1.18 | 796.6 |
| 632 | A-1.8 | B-2.5 | C-2.2 | 1.10 | 770.6 |
| 633 | A-1.8 | B-1.13 | C-2.4 | 1.19 | 795.7 |
| 634 | A-1.20 | B-6.1 | C-2.2 | 1.28 | 801.7 |
| 635 | A-1.20 | B-6.1 | C-2.8 | 1.25 | 801.7 |
| 636 | A-1.20 | B-1.19 | C-2.2 | 1.34 | 815.7 |
| 637 | A-1.19 | B-1.19 | C-2.2 | 1.23 | 811.7 |
| 638 | A-1.18 | B-1.20 | C-2.2 | 1.23 | 810.6 |
| 639 | A-1.18 | B-1.19 | C-2.2 | 1.26 | 810.7 |
| 640 | A-1.24 | B-1.19 | C-2.2 | 1.25 | 810.7 |
| 641 | A-1.22 | B-1.19 | C-2.2 | 1.24 | 811.8 |
| 642 | A-1.24 | B-1.14 | C-2.2 | 1.20 | 792.6 |
| 643 | A-1.18 | B-1.19 | C-2.1 | 1.15 | 812.6 |
| 644 | A-1.24 | B-1.19 | C-2.1 | 1.13 | 812.6 |
| 645 | A-1.22 | B-1.19 | C-2.1 | 1.13 | 813.7 |
| 646 | A-1.22 | B-1.14 | C-14.1 | 1.25 | 807.7 |
| 647 | A-1.1 | B-4.11 | C-1.1 | 1.14 | 731.5 |
| 648 | A-1.24 | B-4.11 | C-1.2 | 1.14 | 758.7 |
| 649 | A-1.19 | B-4.11 | C-1.2 | 1.12 | 759.7 |
| 650 | A-1.1 | B-4.11 | C-1.2 | 1.21 | 757.6 |
| 651 | A-1.7 | B-1.21 | C-2.2 | 1.28 | 795.7 |
| 652 | A-1.24 | B-4.11 | C-2.2 | 1.21 | 772.7 |
| 653 | A-1.7 | B-4.3 | C-2.2 | 1.26 | 823.7 |
| 654 | A-1.8 | B-1.13 | C-1.2 | 1.18 | 751.7 |
| 655 | A-1.24 | B-4.9 | C-2.2 | 1.23 | 810.6 |
| 656 | A-1.22 | B-4.12 | C-2.2 | 1.19 | 787.6 |
| 657 | A-1.22 | B-4.13 | C-2.2 | 1.19 | 790.7 |
| 658 | A-1.24 | B-4.12 | C-2.2 | 1.19 | 786.6 |
| 659 | A-1.24 | B-4.13 | C-2.2 | 1.19 | 789.7 |
| 660 | A-1.24 | B-1.27 | C-2.2 | 1.19 | 810.8 |
| 661 | A-1.24 | B-1.18 | C-18.1 | 1.22 | 864.8 |
| 662 | A-1.24 | B-1.18 | C-18.1 | 1.26 | 864.7 |
| 663 | A-1.24 | B-1.18 | C-2.9 | 1.12 | 808.6 |
| 664 | A-1.18 | B-4.13 | C-2.2 | 1.21 | 789.7 |
| 665 | A-1.30 | B-1.19 | C-2.2 | 1.22 | 810.8 |
| 666 | A-1.19 | B-4.13 | C-2.2 | 1.17 | 790.7 |
| 667 | A-1.31 | B-4.12 | C-2.8 | 1.25 | 814.7 |
| 668 | A-1.24 | B-1.18 | C-2.13 | 1.23 | 798.7 |
| 669 | A-1.24 | B-1.18 | C-2.13 | 1.18 | 798.8 |
| 670 | A-1.24 | B-1.18 | C-19.1 | 1.23 | 848.8 |
| Epimer 1 | | | | | |
| 671 | A-1.24 | B-1.18 | C-19.1 | 1.26 | 848.8 |
| Epimer 2 | | | | | |
| 672 | A-1.24 | B-1.18 | C-19.1 | 1.28 | 848.7 |
| Epimer 3 | | | | | |
| 673 | A-1.24 | B-1.18 | C-19.1 | 1.30 | 848.7 |
| Epimer 4 | | | | | |
| 674 | A-1.24 | B-1.14 | C-19.1 | 1.27 | 868.7 |
| Epimer 1 | | | | | |
| 675 | A-1.24 | B-1.14 | C-19.1 | 1.31 | 868.7 |
| Epimer 2 | | | | | |
| 676 | A-1.24 | B-1.14 | C-19.1 | 1.33 | 868.7 |
| Epimer 3 | | | | | |
| 677 | A-1.24 | B-1.14 | C-19.1 | 1.35 | 868.7 |
| Epimer 4 | | | | | |
| 678 | A-1.8 | B-1.18 | C-2.11 | 1.15 | 794.6 |
| 679 | A-1.8 | B-1.18 | C-2.10 | 1.14 | 794.6 |
| 680 | A-1.1 | B-1.14 | C-1.4 | 1.21 | 765.7 |
| 681 | A-1.19 | B-1.18 | C-2.12 | 1.19 | 809.6 |
| 682 | A-1.18 | B-1.18 | C-18.1 | 1.27 | 864.7 |
| 683 | A-1.8 | B-4.12 | C-2.13 | 1.22 | 786.6 |
| 684 | A-1.28 | B-4.12 | C-2.13 | 1.20 | 802.7 |
| 685 | A-1.30 | B-4.13 | C-2.2 | 1.16 | 789.6 |
| 686 | A-1.28 | B-1.14 | C-1.2 | 1.09 | 768.6 |
| 687 | A-1.14 | B-2.14 | C-1.1 | 1.13 | 714.7 |
| 688 | A-1.6 | B-2.14 | C-1.1 | 1.12 | 728.7 |
| 768 | A-1.1 | B-1.18 | C-1.4 | 1.12 | 745.5 |
| 769 | A-1.28 | B-4.9 | C-2.2 | 1.18 | 800.5 |
| 770 | A-1.28 | B-4.9 | C-2.8 | 1.14 | 800.4 |
| 771 | A-1.28 | B-1.18 | C-1.4 | 1.02 | 736.4 |
| 772 | A-1.28 | B-4.15 | C-1.4 | 1.07 | 750.5 |
| 773 | A-1.8 | B-4.15 | C-18.2 | 1.11 | 748.5 |
| 774 | A-1.1 | B-4.15 | C-18.2 | 1.23 | 773.5 |
| 775 | A-1.8 | B-4.15 | C-18.6 | 1.13 | 784.5 |
| 776 | A-1.28 | B-1.18 | C-2.15 | 1.14 | 804.4 |
| 777 | A-1.24 | B-4.9 | C-2.8 | 1.18 | 810.5 |
| 778 | A-1.38 | B-4.9 | C-2.2 | 1.22 | 810.5 |
| 779 | A-1.38 | B-4.9 | C-2.8 | 1.18 | 810.5 |
| 780 | A-1.22 | B-4.9 | C-2.2 | 1.22 | 811.5 |
| 781 | A-1.18 | B-4.9 | C-2.2 | 1.23 | 810.5 |
| 782 | A-1.19 | B-1.18 | C-22.1 | 1.11 | 910.5 |
| 783 | A-1.28 | B-1.18 | C-22.1 | 1.09 | 899.5 |
| 784 | A-1.28 | B-4.15 | C-18.2 | 1.12 | 764.5 |
| 785 | A-1.28 | B-4.15 | C-18.6 | 1.14 | 800.5 |
| 786 | A-1.28 | B-4.9 | C-1.4 | 1.11 | 774.4 |
| 787 | A-1.38 | B-4.9 | C-1.4 | 1.15 | 784.5 |
| 788 | A-1.1 | B-2.17 | C-1.4 | 0.88 | 754.5 |
| 789 | A-1.28 | B-2.17 | C-1.4 | 0.80 | 745.5 |
| 790 | A-1.24 | B-2.17 | C-1.4 | 0.83 | 755.5 |
| 791 | A-1.28 | B-4.15 | C-2.15 | 1.19 | 818.4 |
| 792* | A-1.28 | B-4.15 | C-2.15 | 1.17 | 818.4 |
| 793 | A-1.19 | B-4.9 | C-2.8 | 1.16 | 811.5 |
| 794 | A-1.19 | B-4.9 | C-22.1 | 1.19 | 948.5 |
| 795 | A-1.28 | B-4.9 | C-22.1 | 1.17 | 937.5 |
| 796 | A-1.28 | B-7.2 | C-2.2 | 0.86 | 796.5 |
| 797 | A-1.24 | B-4.9 | C-1.4 | 1.16 | 784.5 |
| 798 | A-1.18 | B-4.9 | C-1.4 | 1.17 | 784.5 |
| 799 | A-1.19 | B-4.9 | C-1.4 | 1.13 | 785.5 |
| 800 | A-1.22 | B-4.9 | C-1.4 | 1.15 | 785.4 |
| 801 | A-1.19 | B-4.9 | C-18.6 | 1.20 | 835.5 |
| 802* | A-1.19 | B-4.9 | C-18.6 | 1.17 | 835.5 |
| 803 | A-1.19 | B-4.9 | C-18.2 | 1.18 | 799.4 |
| 804 | A-1.37 | B-4.9 | C-22.1 | 1.18 | 948.5 |
| 805 | A-1.30 | B-4.9 | C-2.2 | 1.19 | 810.3 |
| 806 | A-1.37 | B-4.15 | C-2.15 | 1.21 | 829.5 |
| 807 | A-1.8 | B-4.9 | C-22.1 | 1.16 | 921.5 |
| 808 | A-1.22 | B-4.9 | C-22.1 | 1.20 | 948.5 |
| 809 | A-1.28 | B-1.19 | C-22.2 | 1.28 | 910.4 |
| 810* | A-1.28 | B-1.19 | C-22.2 | 1.25 | 910.4 |
| 811 | A-1.19 | B-1.19 | C-22.2 | 1.31 | 921.5 |
| 812* | A-1.19 | B-1.19 | C-22.2 | 1.28 | 921.5 |
| 813 | A-1.19 | B-1.19 | C-22.1 | 1.20 | 948.5 |
| 814* | A-1.19 | B-1.19 | C-22.1 | 1.18 | 948.5 |
| 815 | A-1.28 | B-1.19 | C-22.1 | 1.18 | 937.5 |
| 816* | A-1.28 | B-1.19 | C-22.1 | 1.16 | 937.5 |
| 817 | A-1.37 | B-4.9 | C-1.2 | 1.12 | 797.5 |
| 818 | A-1.28 | B-4.9 | C-1.2 | 1.11 | 786.5 |
| 819 | A-1.28 | B-4.9 | C-18.6 | 1.17 | 824.4 |
| 820 | A-1.19 | B-4.15 | C-22.2 | 1.26 | 897.5 |
| 821 | A-1.36 | B-4.9 | C-2.2 | 1.15 | 801.5 |
| 822 | A-1.28 | B-4.14 | C-2.2 | 1.11 | 796.5 |
| 823 | A-1.37 | B-7.2 | C-2.2 | 0.87 | 807.5 |
| 824 | A-1.8 | B-7.2 | C-1.4 | 0.80 | 754.5 |
| 825 | A-1.28 | B-7.2 | C-1.4 | 0.81 | 770.5 |
| 826 | A-1.22 | B-7.2 | C-1.4 | 0.83 | 781.5 |
| 827 | A-1.28 | B-1.19 | C-23.1 | 1.13 | 879.5 |
| 828 | A-1.35 | B-4.15 | C-18.4 | 1.26 | 787.5 |
| 829 | A-1.37 | B-4.9 | C-2.2 | 1.19 | 811.5 |
| 851 | A-1.19 | B-1.19 | C-20.5 | 1.09 | 881.4 |
| 852 | A-1.8 | B-1.23 | C-8.2 | 1.11 | 862.4 |
| 853 | A-1.1 | B-1.23 | C-8.2 | 1.21 | 887.4 |
| 854 | A-1.28 | B-1.18 | C-18.1 | 1.14 | 854.4 |
| 855 | A-1.8 | B-1.18 | C-27.2 | 1.13 | 826.4 |
| 856 | A-1.28 | B-1.18 | C-27.2 | 1.14 | 842.4 |
| 857 | A-1.19 | B-1.18 | C-2.16 | 1.13 | 817.4 |
| 858 | A-1.8 | B-1.18 | C-27.1 | 1.19 | 832.5 |
| 859 | A-1.19 | B-1.18 | C-24.1 | 1.17 | 891.4 |
| 860 | A-1.1 | B-4.16 | C-1.4 | 1.12 | 760.4 |
| 861 | A-1.19 | B-1.18 | C-25.1 | 1.02 | 844.4 |
| 862 | A-1.24 | B-4.16 | C-1.4 | 1.07 | 761.4 |
| 863 | A-1.19 | B-1.18 | C-2.17 | 1.08 | 787.4 |
| 864 | A-1.19 | B-1.18 | C-20.2 | 1.07 | 869.4 |
| 865 | A-1.1 | B-4.15 | C-29.1 | 1.30 | 801.5 |
| 866 | A-1.8 | B-4.15 | C-29.1 | 1.20 | 776.4 |
| 867 | A-1.19 | B-1.18 | C-22.4 | 1.17 | 949.5 |
| 868 | A-1.28 | B-1.18 | C-22.4 | 1.15 | 938.4 |
| 869 | A-1.8 | B-4.17 | C-2.2 | 1.08 | 776.4 |
| 870 | A-1.24 | B-4.17 | C-2.2 | 1.13 | 802.4 |
| 871 | A-1.38 | B-1.18 | C-22.4 | 1.19 | 948.5 |
| 872 | A-1.36 | B-1.14 | C-1.2 | 1.00 | 769.4 |
| 873 | A-1.1 | B-4.15 | C-18.10 | 1.31 | 815.5 |
| 874 | A-1.8 | B-4.15 | C-29.2 | 1.20 | 824.4 |
| 875 | A-1.1 | B-4.15 | C-29.2 | 1.29 | 849.5 |
| 876 | A-1.8 | B-4.15 | C-18.10 | 1.22 | 790.4 |
| 877 | A-1.28 | B-4.15 | C-18.9 | 1.07 | 808.4 |
| 878 | A-1.28 | B-4.15 | C-18.10 | 1.23 | 806.4 |
| 879 | A-1.19 | B-4.9 | C-25.1 | 1.10 | 882.4 |
| 880 | A-1.28 | B-4.9 | C-25.1 | 1.08 | 871.4 |
| 881 | A-1.19 | B-1.18 | C-20.4 | 1.16 | 885.5 |
| 882 | A-1.19 | B-1.18 | C-26.1 | 1.06 | 890.4 |
| 883 | A-1.19 | B-1.18 | C-20.3 | 1.18 | 897.5 |
| 884 | A-1.28 | B-1.18 | C-20.3 | 1.16 | 886.4 |
| 885 | A-1.19 | B-1.19 | C-26.2 | 1.11 | 947.4 |
| 886 | A-1.19 | B-1.19 | C-25.2 | 1.23 | 908.4 |
| 887 | A-1.19 | B-1.19 | C-22.5 | 1.14 | 962.4 |
| 888 | A-1.37 | B-4.14 | C-2.2 | 1.07 | 807.4 |
| 889 | A-1.19 | B-7.2 | C-22.2 | 0.92 | 917.4 |
| 890 | A-1.37 | B-7.2 | C-22.2 | 0.92 | 917.4 |
| 891 | A-1.40 | B-4.9 | C-22.1 | 1.06 | 964.39 |
| 892 | A-1.39 | B-4.9 | C-22.1 | 1.07 | 964.4 |
| 893 | A-1.37 | B-4.9 | C-18.6 | 1.12 | 835.4 |
| 894 | A-1.8 | B-4.9 | C-18.9 | 1.09 | 816.4 |
| 895 | A-1.28 | B-4.9 | C-18.9 | 1.10 | 832.4 |
| 896 | A-1.37 | B-4.9 | C-18.9 | 1.12 | 843.4 |
| 897 | A-1.28 | B-4.15 | C-22.2 | 1.17 | 886.4 |
| 898 | A-1.28 | B-7.2 | C-22.2 | 0.91 | 906.4 |
| 899 | A-1.28 | B-4.18 | C-2.2 | 1.08 | 800.4 |
| 900 | A-1.19 | B-7.2 | C-1.2 | 0.78 | 793.4 |
| 901 | A-1.28 | B-7.2 | C-1.2 | 0.76 | 782.4 |
| 902 | A-1.37 | B-4.15 | C-22.2 | 1.18 | 897.4 |
| 903 | A-1.41 | B-4.15 | C-22.2 | 1.21 | 906.4 |
| 904 | A-1.41 | B-7.2 | C-1.2 | 0.80 | 802.4 |
| 905 | A-1.19 | B-7.2 | C-22.1 | 0.84 | 944.4 |
| 906 | A-1.28 | B-7.2 | C-22.1 | 0.83 | 933.4 |
| 907 | A-1.37 | B-7.2 | C-22.1 | 0.84 | 944.4 |
| 908 | A-1.44 | B-4.9 | C-22.2 | 1.08 | 931.4 |
| 909 | A-1.28 | B-4.14 | C-22.2 | 1.16 | 906.4 |
| 910 | A-1.24 | B-4.15 | C-18.9 | 1.10 | 818.4 |
| 911 | A-1.36 | B-4.9 | C-1.2 | 1.02 | 787.4 |
| 912 | A-1.28 | B-4.7 | C-2.2 | 0.97 | 782.4 |
| 913 | A-1.28 | B-7.2 | C-18.9 | 0.83 | 828.4 |
| 914 | A-1.36 | B-7.2 | C-18.9 | 0.80 | 829.4 |
| 915 | A-1.37 | B-7.2 | C-18.9 | 0.83 | 839.5 |
| 916 | A-1.36 | B-4.9 | C-22.1 | 1.08 | 938.4 |
| 917 | A-1.36 | B-7.2 | C-22.1 | 0.82 | 934.4 |
| 918 | A-1.36 | B-7.2 | C-22.2 | 0.89 | 907.4 |
| 919 | A-1.36 | B-4.15 | C-22.2 | 1.15 | 887.4 |
| 920 | A-1.36 | B-4.14 | C-2.2 | 1.02 | 797.4 |
| 921 | A-1.37 | B-7.2 | C-22.3 | 0.86 | 899.4 |
| 922 | A-1.36 | B-4.9 | C-5.4 | 1.10 | 837.4 |
| 923 | A-1.37 | B-4.14 | C-22.2 | 1.17 | 917.4 |
| 924 | A-1.37 | B-4.9 | C-22.2 | 1.21 | 921.4 |
| 925 | A-1.42 | B-4.9 | C-22.2 | 1.28 | 911.3 |
| 926 | A-1.28 | B-4.9 | C-2.4 | 1.06 | 830.4 |
| 927 | A-1.36 | B-4.14 | C-22.2 | 1.14 | 907.4 |
| 928* | A-1.37 | B-4.14 | C-2.2 | 1.06 | 807.4 |
| 929 | A-1.36 | B-4.9 | C-18.9 | 1.08 | 833.4 |
| 930 | A-1.36 | B-1.19 | C-2.2 | 1.09 | 801.4 |
| 931 | A-1.28 | B-7.2 | C-18.6 | 0.82 | 820.4 |
| 932 | A-1.36 | B-7.2 | C-18.6 | 0.80 | 821.4 |
| 933 | A-1.37 | B-7.2 | C-18.6 | 0.84 | 831.4 |
| 934 | A-1.36 | B-4.9 | C-22.2 | 1.18 | 911.3 |
| 935 | A-1.19 | B-4.14 | C-22.2 | 1.18 | 917.4 |
| 936 | A-1.22 | B-4.14 | C-22.2 | 1.19 | 917.4 |
| 937 | A-1.41 | B-4.9 | C-22.2 | 1.24 | 930.4 |
| 938 | A-1.36 | B-7.2 | C-2.15 | 0.84 | 839.4 |
| 939* | A-1.36 | B-7.2 | C-2.15 | 0.82 | 839.4 |
| 940* | A-1.36 | B-4.9 | C-2.15 | 1.11 | 843.3 |
| 941 | A-1.36 | B-4.9 | C-2.15 | 1.13 | 843.3 |
| 942 | A-1.36 | B-4.15 | C-2.15 | 1.10 | 819.4 |
| 943 | A-1.39 | B-4.15 | C-22.2 | 1.16 | 913.4 |
| 944 | A-1.43 | B-4.9 | C-22.2 | 1.23 | 893.3 |
| 945 | A-1.46 | B-4.15 | C-22.2 | 1.21 | 904.4 |
| 946 | A-1.8 | B-4.9 | C-18.6 | 1.10 | 808.4 |
| 947 | A-1.36 | B-4.9 | C-18.6 | 1.08 | 825.4 |
| 948 | A-1.28 | B-1.18 | C-22.2 | 1.13 | 872.4 |
| 949 | A-1.36 | B-1.18 | C-22.2 | 1.11 | 873.4 |
| 950 | A-1.19 | B-7.2 | C-2.15 | 0.87 | 849.4 |
| 951 | A-1.22 | B-7.2 | C-2.15 | 0.88 | 849.4 |
| 952 | A-1.22 | B-7.2 | C-5.4 | 0.85 | 843.4 |
| 953 | A-1.28 | B-7.2 | C-5.4 | 0.83 | 832.4 |
| 954 | A-1.36 | B-7.2 | C-5.4 | 0.81 | 833.4 |
| 955 | A-1.37 | B-7.2 | C-5.4 | 0.84 | 843.4 |
| 956 | A-1.24 | B-7.2 | C-2.15 | 0.89 | 848.4 |
| 957 | A-1.28 | B-7.2 | C-2.15 | 0.86 | 838.4 |
| 958 | A-1.18 | B-7.2 | C-2.15 | 0.90 | 848.4 |
| 959 | A-1.37 | B-7.2 | C-2.15 | 0.87 | 849.4 |
| 960 | A-1.46 | B-7.2 | C-2.15 | 0.90 | 856.4 |
| 961 | A-1.45 | B-4.15 | C-22.2 | 1.12 | 913.4 |
| 962 | A-1.45 | B-4.9 | C-22.2 | 1.14 | 937.4 |
| 963 | A-1.46 | B-4.9 | C-1.2 | 1.10 | 804.4 |
| 964 | A-1.22 | B-4.9 | C-22.2 | 1.23 | 921.4 |
| 965 | A-1.8 | B-4.9 | C-22.2 | 1.19 | 894.4 |
| 966 | A-1.39 | B-4.9 | C-22.2 | 1.17 | 937.4 |
| 967 | A-1.46 | B-4.9 | C-18.6 | 1.15 | 842.3 |
| 968 | A-1.46 | B-4.15 | C-18.6 | 1.12 | 818.4 |
| 969 | A-1.46 | B-4.9 | C-5.4 | 1.17 | 854.3 |
| 970 | A-1.46 | B-7.2 | C-18.6 | 0.86 | 838.4 |
| 971 | A-1.36 | B-4.9 | C-25.3 | 1.18 | 899.4 |
| 972 | A-1.28 | B-4.9 | C-25.3 | 1.20 | 898.4 |
| 973 | A-1.46 | B-4.9 | C-2.2 | 1.16 | 818.4 |
| 974 | A-1.28 | B-2.5 | C-2.15 | 1.09 | 828.4 |
| 975 | A-1.36 | B-4.14 | C-2.15 | 1.08 | 839.4 |

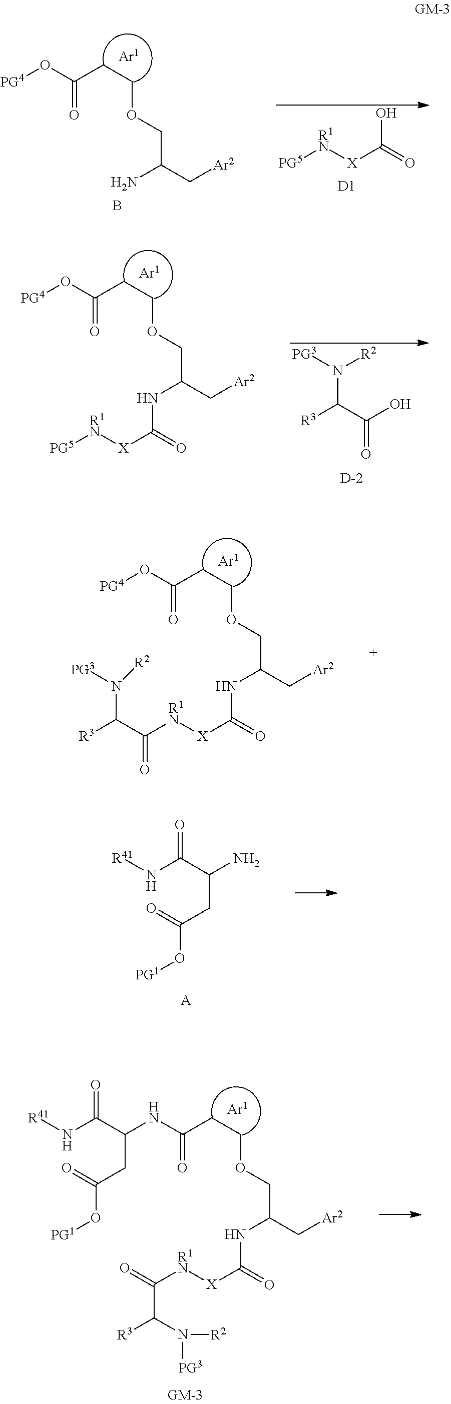

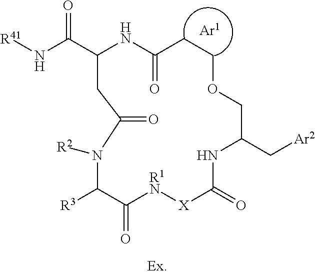

Listed in Table MC-3 below are compounds of general formula (I) that are prepared from the corresponding building blocks A, B, D1, and D2 in analogy to the corresponding General Method 3 (GM-3) (see Ex. 689).

TABLE MC-3
(GM-3)

| | | | | | t_{R} [min] | MS-data m/z |
|---|---|---|---|---|---|---|
| Ex. | A | B | D1 | D2 | LC-MS Method H | [M + H]⁺ |
| 690 | A-1.8 | B-1.19 | D1-3.0 | Boc-N-methyl-L-leucine | 1.18 | 784.6 |
| 691 | A-1.19 | B-1.19 | D1-3.0 | Boc-N-methyl-L-leucine | 1.22 | 811.7 |
| 692 | A-1.22 | B-1.19 | D1-3.0 | Boc-N-methyl-L-leucine | 1.23 | 811.6 |
| 693 | A-1.19 | B-1.19 | D1-4.1 | Boc-N-methyl-L-leucine | 1.41 | 853.8 |
| 694 | A-1.19 | B-1.19 | D1-4.1 | Boc-N-methyl-L-leucine | 1.36 | 853.8 |
| 695* | A-1.8 | B-1.14 | D1-4.2 | Boc-N-methyl-L-leucine | 1.28 | 866.8 |
| 696 | A-1.22 | B-1.19 | D1-4.5 | Boc-N-methyl-L-leucine | 1.45 | 867.8 |
| 697 | A-1.24 | B-1.19 | D1-4.5 | Boc-N-methyl-L-leucine | 1.45 | 866.8 |
| 698 | A-1.8 | B-1.14 | D1-4.2 | Boc-N-methyl-L-leucine | 1.32 | 866.7 |
| 699 | A-1.8 | B-1.14 | D1-4.6 | Boc-N-methyl-L-leucine | 1.15 | 790.7 |

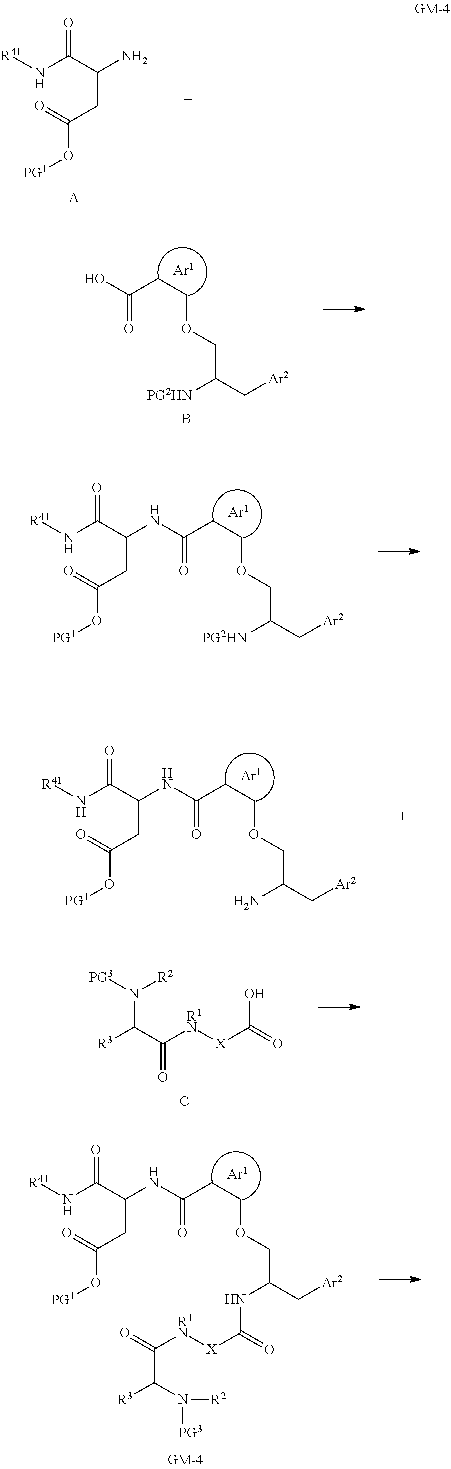

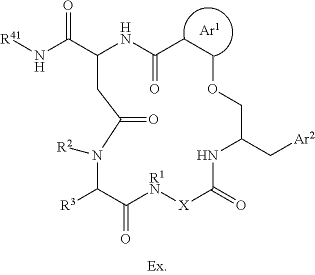

Listed in Table MC-4 below are compounds of general formula (I) that are prepared from the corresponding building blocks A, B, and C in analogy to the corresponding General Method 4 (GM-4) (see Ex. 700 or Ex. 976 depending on the Protecting Group strategy).

TABLE MC-4
(GM-4)

| | | | | t_{R} [min] | |
|---|---|---|---|---|---|
| | | | | LC-MS | MS-data m/z |
| Ex. | A | B | C | Method H | [M + H]⁺ |
| 701 | A-2.3 | B-1.24 | C-16.1 | 1.26 | 847.8 |
| 702 | A-2.4 | B-Acid-6 | C-16.8 | 1.19 | 844.8 |
| 703 | A-2.4 | B-1.25 | C-16.6 | 1.22 | 880.7 |
| 704 | A-2.4 | B-Acid-6 | C-16.2 | 1.21 | 858.6 |
| 705 | A-2.4 | B-Acid- 6 | C-16.3 | 1.21 | 858.8 |
| 706 | A-2.4 | B-Acid-6 | C-16.9 | 1.23 | 838.6 |
| 707 | A-2.4 | B-Acid-6 | C-16.9 | 1.26 | 838.8 |
| 708 | A-2.4 | B-Acid-6 | C-17.4 | 1.17 | 780.8 |
| 709 | A-2.4 | B-Acid-6 | C-17.2 | 1.38 | 822.7 |
| 710 | A-2.4 | B-Acid-6 | C-17.3 | 1.27 | 794.6 |
| 711 | A-2.4 | B-Acid-6 | C-17.5 | 1.16 | 802.6 |
| 712 | A-2.2 | B-Acid-5 | C-16.3 | 1.38 | 896.8 |
| 976 | A-1.22 | B-Acid-8 | C-18.9 | 1.14 | 843.4 |
| 977 | A-1.28 | B-Acid-9 | C-28.1 | 1.07 | 872.4 |
| 978 | A-1.28 | B-Acid-9 | C-28.2 | 1.00 | 887.4 |
| 979 | A-1.28 | B-Acid-9 | C-18.7 | 1.12 | 790.4 |
| 980 | A-1.28 | B-Acid-9 | C-18.8 | 1.20 | 804.4 |

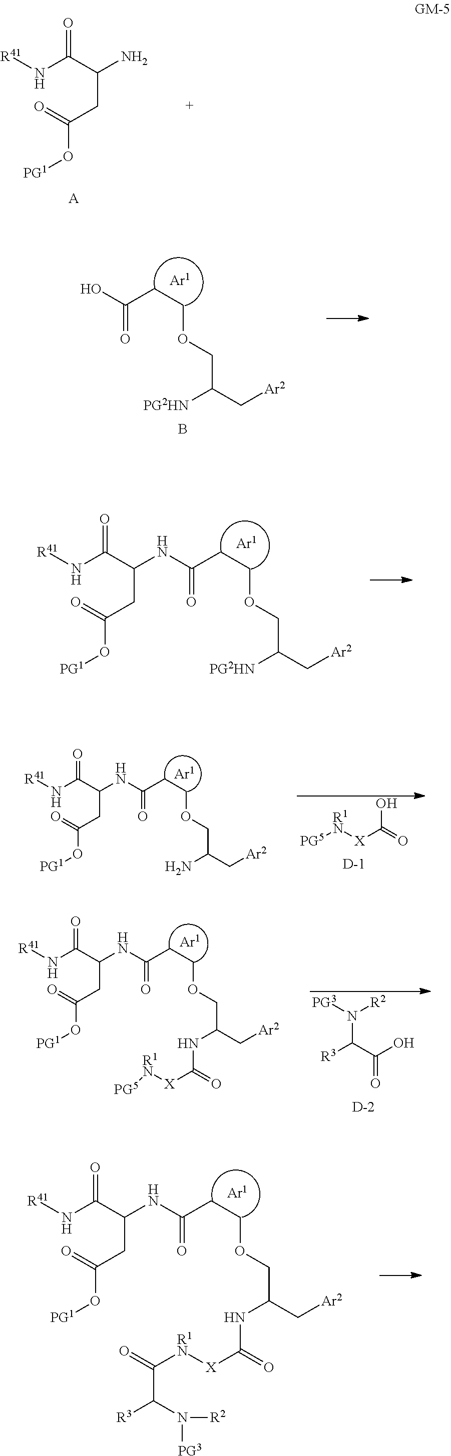

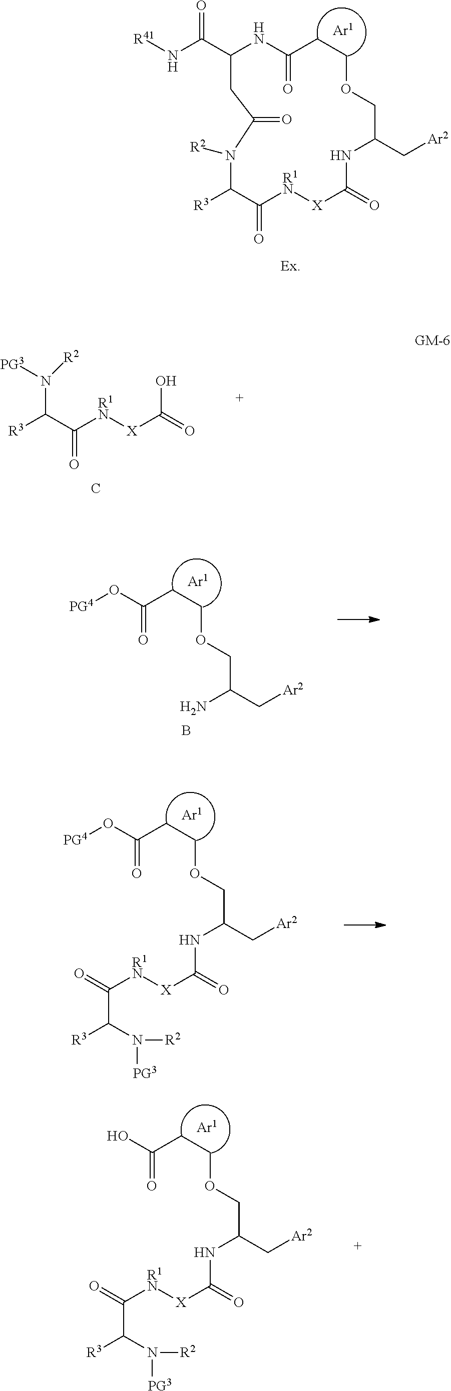

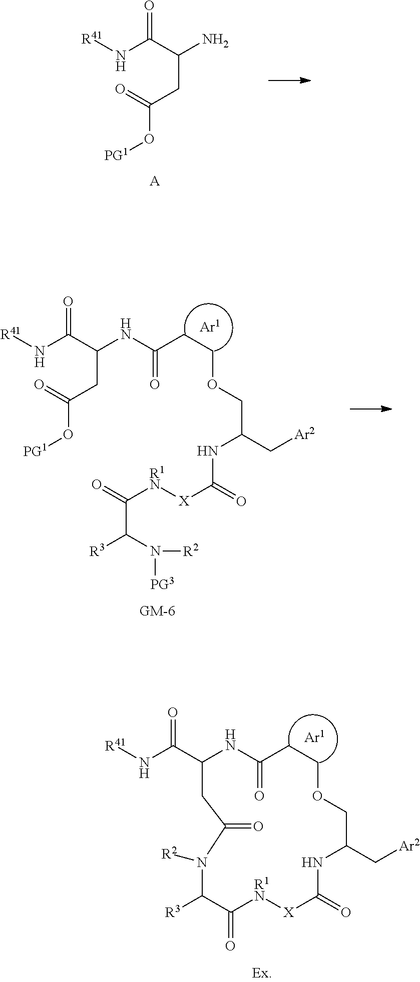

Listed in Table MC-5 below are compounds of general formula (I) that are prepared from the corresponding building blocks in analogy to the corresponding General Methods, GM-5 (see Ex. 713), GM-6 (see Ex. 722), GM-7 (see Ex. 724), GM-8 (see Ex. 725) or GM-9 (see Ex. 761).

TABLE MC-5
(GM-5 to GM-9)

| | | | | | | | t_{R} [min] | MS-data |
|---|---|---|---|---|---|---|---|---|
| | | | | | | | LC-MS | m/z |
| Ex. | GM | A | B | C | D1 | D2 | Method H | [M + H]⁺ |
| 714 | GM-5 | A-2.4 | B-Acid-5 | | D1-1.1 | D2-3.1 | 1.34 | 845.8 |
| 715 | GM-5 | A-2.3 | B-Acid-5 | | D1-1.1 | D2-3.1 | 1.43 | 870.8 |
| 716 | GM-5 | A-2.4 | B-Acid-5 | | D1-1.1 | D2-3.1 | 1.32 | 845.7 |
| 717 | GM-5 | A-2.3 | B-Acid-5 | | D1-1.2 | D2-3.1 | 1.44 | 836.8 |
| 718 | Isolated as sideproduct from Ex. 717 | | | | | | 1.20 | 780.7 |
| 719 | GM-5 | A-2.4 | B-Acid-5 | | D1-1.2 | D2-3.1 | 1.33 | 811.7 |
| 720 | Isolated as sideproduct from Ex. 719 | | | | | | 1.06 | 755.6 |
| 721 | GM-6 | A-1.8 | B-1.19 | C-17.3 | | | 1.31 | 812.6 |
| 723* | GM-6 | A-1.8 | B-1.19 | C-17.1 | | | 1.27 | 834.7 |
| 726 | GM-8 | A-1.1 | B-1.13 | C-7.1 | | | 0.87 | 791.8 |
| 727 | GM-8 | A-1.8 | B-1.14 | C-7.1 | | | 0.70 | 767.6 |
| 728 | Isolated as sideproduct from Ex. 422 | | | | | | 1.32 | 865.7 |
| 762 | GM-9 | | B-2.5 | C-1.2 | | | 1.14 | 784.6 |
| 763 | GM-9 | | B-1.14 | C-1.2 | | | 1.22 | 829.8 |
| 764 | GM-9 | | B-1.14 | C-1.2 | | | 1.17 | 793.7 |

### Post Modifications (PM)

### PM-1: Sulfonamide Formation

### Example 729: (4aR,7R,18S,22S)-7-benzyl-22-isobutyl-N-(3-methoxyphenethyl)-21-methyl-5,16,20,23-tetraoxo-3-(phenylsulfonyl)-2,3,4,4a,5,6,7,8,16,17,18,19,20,21,22,23-hexadecahydro-1H-naphtho[1,2-p]pyrazino[2,1-f][1]oxa[4,7,10,14]tetraazacycloheptadecine-18-carboxamide

Benzenesulfonyl chloride (0.04 mL, 0.15 mmol) is added to a RT soln. of Ex. 726 (40 mg, 0.0506 mmol) and TEA (0.0213 mL, 0.152 mmol) in DCM (0.5 mL) and the mix. is stirred at RT overnight. The mix. is concentrated and the residue is taken up in MeCN/DMF and directly purified by prep. HPLC (basic) to yield the title compound (18 mg, 38%) as a white solid. LC-MS B: t_{R}=1.14 min; [M+H]⁺=931.45.

### PM-2: Amide Coupling

### Example 750: (4aR,7R,18S,22S)-3-Acetyl-7-benzyl-22-isobutyl-N-(3-methoxyphenethyl)-21-methyl-5,16,20,23-tetraoxo-2,3,4,4a,5,6,7,8,16,17,18,19,20,21,22,23-hexadecahydro-1H-naphtho[1,2-p]pyrazino[2,1-f][1]oxa[4,7,10,14]tetraazacycloheptadecine-18-carboxamide

(H)ATU (22.2 mg, 0.0582 mmol) is added to a RT soln. of Ex. 726 (40 mg, 0.0506 mmol), DIPEA (20 μL, 0.126 mmol), and acetic acid (1.15 eq) in DMF (0.5 mL). The RM is stirred at RT for 1 h. The RM is directly purified by prep. HPLC (basic) to yield the title compound (28 mg, 66%) as a white solid. LC-MS B: t_{R}=1.03 min; [M+H]⁺=833.41.

### PM-3A: Carbamate Formation

### Example 754: Methyl (4aR,7R,18S,22S)-7-benzyl-22-isobutyl-18-((3-methoxyphenethyl)carbamoyl)-21-methyl-5,16,20,23-tetraoxo-1,2,4,4a,5,6,7,8,16,17,18,19,20,21,22,23-hexadecahydro-3H-naphtho[1,2-p]pyrazino[2,1-f][1]oxa[4,7,10,14]tetraazacycloheptadecine-3-carboxylate

Methyl chloroformate (ca 6 μL mL, 0.0759 mmol) is added to a RT soln. of Ex. 726 (30 mg, 0.0379 mmol) and TEA (16 μL, 0.114 mmol) in DCM (0.5 mL), and the RM is stirred at RT for 1.5 h. The RM is diluted with DMF and directly purified by prep. HPLC (basic) to yield the title compound (22 mg, 69%) as a white solid. LC-MS B: t_{R}=1.08 min; [M+H]⁺=849.44.

### PM-3B: Carbamate Formation

### Example 756: Tetrahydro-2H-pyran-4-yl (4aR,7R,18S,22S)-7-benzyl-22-isobutyl-18-((3-methoxyphenethyl)carbamoyl)-21-methyl-5,16,20,23-tetraoxo-1,2,4,4a,5,6,7,8,16,17,18,19,20,21,22,23-hexadecahydro-3H-naphtho[1,2-p]pyrazino[2,1-f][1]oxa[4,7,10,14]tetraazacycloheptadecine-3-carboxylate

Tetrahydro-2H-pyran-4-ol (6 μL, 0.0607 mmol) is added to a RT suspension of N,N′-disuccinimidyl carbonate (24.5 mg, 0.091 mmol) and TEA (26 μL, 0.182 mmol) in MeCN (0.4 mL). The RM is stirred at RT overnight, then to the RM is added a soln. of Ex. 726 (40 mg, 0.0506 mmol) in MeCN (0.4 mL) and the RM is stirred at RT for 3 h. The mix. is diluted with MeCN and directly purified by prep. HPLC (basic) to yield the title compound (28 mg, 61%) as a white solid. LC-MS B: t_{R}=1.10 min; [M+H]⁺=919.53.

### PM-4: Boc-protected Sulfonimidamide

### Example 757: tert-Butyl (((4aR,7R,18S,22S)-7-benzyl-22-isobutyl-18-((3-methoxyphenethyl)carbamoyl)-21-methyl-5,16,20,23-tetraoxo-1,2,4,4a,5,6,7,8,16,17,18,19,20,21,22,23-hexadecahydro-3H-naphtho[1,2-p]pyrazino[2,1-f][1]oxa[4,7,10,14]tetraazacycloheptadecin-3-yl)(oxo)(phenyl)-16-sulfaneylidene)carbamate

Step 1: NaOCl 10% (250 mL, 413 mmol) is added dropwise at a 0° C. soln. of t-butanol (31 mL, 327 mmol) and AcOH (20 mL, 350 mmol) (slightly exothermic). The resulting emulsion is stirred at 0° C. for 10 min then transferred to a separating funnel. The aq. layer is discarded and the remaining yellow liquid is washed with water (2×), then dried (CaCl₂), filtered to yield tert-butyl hypochlorite (8.5 g, 21%) as a yellow non-viscous oil.

Step 2: Lithium bis(trimethylsilyl)amide soln. (1.0 M in THF, 1.35 mL, 1.35 mmol) is added dropwise to a 0° C. soln. of benzenesulfinamide (100 mg, 0.673 mmol) in THF (1.5 mL). The brown turbid mix. is stirred at 0° C. for 1 h, then a soln. of di-tert-butyl dicarbonate (0.156 mL, 0.673 mmol) in THF (0.5 mL) is added dropwise. The ice bath is removed, and the soln. stirred at RT for 15 min. To the RM is added water (10 mL) and DCM (100 mL). The layers are separated and the aq. layer is extracted with DCM (2×75 mL). The combined org. layers are washed with brine (50 mL), dried (MgSO₄), filtered, and concentrated. Purification by FC (eluting with 15% to 55% EtOAc in hept, R_{f}=0.24 in EtOAc/hept 3:7) yields tert-butyl (phenylsulfinyl)carbamate (140 mg, 86%) as a colourless oil. LC-MS B: t_{R}=0.78 min; [M+H]⁺=241.97.

Step 3: Freshly prepared tert-butyl hypochlorite (41.2 mg, 0.379 mmol) is added to a 0° C. soln. of tert-butyl (phenylsulfinyl)carbamate (54.9 mg, 0.228 mmol) in DCM (3 mL). The resulting mix. is stirred at 0° C. for 30 min, then a RT soln. of Ex. 726 (150 mg, 0.19 mmol) and DIPEA (0.0974 mL, 0.569 mmol) in DCM (5 mL) is added dropwise at 0° C. The ice bath is removed and the RM is stirred at RT for 30 min. To the reaction is added water (10 mL) and DCM (50 mL) and the layers are separated. The aq. layer is extracted with DCM (2×35 mL) and the combined org. layers are washed with brine (10 mL), dried (MgSO₄), filtered, and concentrated. Purification by prep. HPLC (basic) yields Ex. 757 (70 mg, 36%) as a white solid. LC-MS B: t_{R}=1.19 min; [M+H]⁺=1030.28.

### PM-5: Sulfonimidamide

### Example 758: (4aR,7R,18S,22S)-7-Benzyl-22-isobutyl-N-(3-methoxyphenethyl)-21-methyl-5,16,20,23-tetraoxo-3-(phenylsulfonimidoyl)-2,3,4,4a,5,6,7,8,16,17,18,19,20,21,22,23-hexadecahydro-1H-naphtho[1,2-p]pyrazino[2,1-f][1]oxa[4,7,10,14]tetraazacycloheptadecine-18-carboxamide

Iodotrimethylsilane (9 μL, 0.058 mmol) is added to a RT soln. of Ex. 757 (30 mg, 0.029 mmol) in MeCN (0.5 mL) and the RM is stirred at RT for 5 min. The RM is directly purified by prep. HPLC (basic) to yield the title compound (44 mg, 81%) as a white solid. LC-MS B: t_{R}=1.08/1.09 min; [M+H]⁺=930.64.

### PM-6: Nitrile Formation

### Example 759: (4aR,7R,18S,22S)-7-Benzyl-N-(3-cyanophenethyl)-22-isobutyl-21-methyl-5,16,20,23-tetraoxo-1,2,4,4a,5,6,7,8,16,17,18,19,20,21,22,23-hexadecahydronaphtho[1,2-p][1,4]oxazino[3,4-f][1]oxa[4,7,10,14]tetraazacycloheptadecine-18-carboxamide

A mix. of Ex. 378 (20 mg, 0.02 mmol) and CuCN (9 mg, 0.1 mmol) in pyridine (0.5 mL) is heated at 140° C. for 72 h. The RM is directly purified by prep. HPLC (basic) to give the title compound as a white solid. LC-MS B: t_{R}=1.05 min; [M+H]⁺=787.28.

### PM-7: N-Oxide Formation

### Example 760: (9S,13S,19aR,22R)-22-Benzyl-13-isobutyl-9-((3-methoxyphenethyl)carbamoyl)-12-methyl-7,11,14,20-tetraoxo-7,8,9,10,11,12,13,14,17,18,19,19a,20,21,22,23-hexadecahydro-16H-pyrido[2′,1′:6,7][1]oxa[4,7,10,14]tetraazacycloheptadecino[17,16-c]quinoline 2-oxide

mCPBA (11 mg, 0.05 mmol) is added to a 0° C. soln. of Ex. 365 (30 mg, 0.04 mmol) in DCM (0.5 mL) and the RM is warmed to RT and stirred for 4 h. The RM is diluted with DCM and quenched with a sat. aq. Na₂S₂O₃ soln. The layers are separated and the aq. phase is re-extracted with DCM (2×). The combined org. extracts are washed with brine, dried over Na₂SO₄, filtered and evaporated in vacuo. The crude product is purified by prep. HPLC (acidic) to give the title compound as a white solid. LC-MS B: t_{R}=1.03 min; [M+H]⁺=807.36.

Listed in Table MC-6 below are compounds of formula (I) that are prepared using one of the above described post modification methods (PM) using the corresponding starting materials. Conditions may vary slightly.

TABLE MC-6

| | | | | t_{R} [min] | |
|---|---|---|---|---|---|
| | | SM-A | | LC-MS | MS-data m/z |
| Ex. | PM | (Ex.) | SM-B | Method H | [M + H]⁺ |
| 730 | PM-1 | Ex. 726 | SCI-20 | 0.90 | 982.8 |
| 731 | PM-1 | Ex. 726 | SCI-13 | 1.24 | 869.7 |
| 732 | PM-1 | Ex. 726 | SCI-6 | 1.45 | 1015.7 |
| 733 | PM-1 | Ex. 726 | SCI-14 | 1.27 | 913.7 |
| 734 | PM-1 | Ex. 726 | SCI-17 | 1.28 | 939.8 |
| 735 | PM-1 | Ex. 726 | SCI-16 | 0.89 | 926.8 |
| 736 | PM-1 | Ex. 726 | SCI-11 | 1.37 | 945.8 |
| 737 | PM-1 | Ex. 726 | SCI-10 | 1.24 | 988.8 |
| 738 | PM-1 | Ex. 726 | SCI-12 | 1.36 | 994.8 [M − H]⁻ |
| 739 | PM-1 | Ex. 726 | SCI-5 | 1.37 | 961.8 |
| 740 | PM-1 | Ex. 726 | SCI-7 | 1.37 | 979.8 |
| 741 | PM-1 | Ex. 726 | SCI-9 | 1.37 | 1002.8 |
| 742 | PM-1 | Ex. 726 | SCI-2 | 1.27 | 930.8 [M − H]⁻ |
| 743 | PM-1 | Ex. 726 | SCI-15 | 1.30 | 927.8 |
| 744 | PM-1 | Ex. 726 | SCI-8 | 1.31 | 962.7 |
| 745 | PM-1 | Ex. 726 | SCI-19 | 0.98 | 968.8 |
| 746 | PM-1 | Ex. 726 | SCI-18 | 1.28 | 953.8 |
| 747 | PM-1 | Ex. 726 | SCI-3 | 1.28 | 932.7 |
| 748 | PM-1 | Ex. 727 | SCI-9 | 1.19 | 978.8 |
| 749 | PM-1 | Ex. 727 | SCI-6 | 1.28 | 991.7 |
| 751 | PM-2 | Ex. 726 | isovaleric acid | 1.32 | 875.8 |
| 752 | PM-2 | Ex. 726 | 3-methoxypropanoic acid | 1.21 | 877.8 |
| 753 | PM-2 | Ex. 726 | pyrazinecarboxylic acid | 1.20 | 897.8 |
| 755 | PM-3A | Ex. 726 | ethyl chloroformate | 1.30 | 863.8 |
| 981 | PM-7 | Ex. 809 | | 1.15 | 926.3 |
| 982 | PM-7 | Ex. 909 | | 1.11 | 922.4 |
| 983 | PM-7 | Ex. 934 | | 1.10 | 927.3 |

Listed in the Table of Examples below are example compounds of formula (I) prepared according to the above described methods. The configuration at stereocentres that are not mentioned in the compound name are unknown however only one epimer is present.

TABLE of
Examples

| Ex. | Compound of Formula (I) |
|---|---|
| 1 | (7R,13S,17RS)-N-(2-(benzo[d][1,3]dioxol-5-yl)ethyl)-7-benzyl-11-(3,3-dimethylbutyl)-13-isobutyl-14- |
| | methyl-9,12,15,19-tetraoxo-6,7,8,9,10,11,12,13,14,15,16,17,18,19-tetradecahydro- |
| | [1,3]dioxolo[4′,5′:4,5]benzo[1,2-p][1]oxa[4,7,10,14]tetraazacycloheptadecine-17-carboxamide |
| 2 | (7R,13S,17S)-N-(2-(benzo[d][1,3]dioxol-5-yl)ethyl)-7-benzyl-11-(2-cyclohexylethyl)-13-isobutyl-14- |
| | methyl-9,12,15,19-tetraoxo-6,7,8,9,10,11,12,13,14,15,16,17,18,19-tetradecahydro- |
| | [1,3]dioxolo[4′,5′:4,5]benzo[1,2-p][1]oxa[4,7,10,14]tetraazacycloheptadecine-17-carboxamide |
| 3 | (3R,9S,13S)-3-benzyl-N-(2-(chroman-6-yl)ethyl)-9-isobutyl-17,18-dimethoxy-7,10-dimethyl-5,8,11,15- |
| | tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 4 | (3R,9S,13S)-N-(2-(benzofuran-6-yl)ethyl)-3-benzyl-9-isobutyl-17,18-dimethoxy-7,10-dimethyl-5,8,11,15- |
| | tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 5 | (7R,13S,17RS)-N-(2-(benzo[d][1,3]dioxol-5-yl)ethyl)-7-benzyl-13-isobutyl-14-methyl-9,12,15,19- |
| | tetraoxo-11-phenethyl-6,7,8,9,10,11,12,13,14,15,16,17,18,19-tetradecahydro- |
| | [1,3]dioxolo[4′,5′:4,5]benzo[1,2-p][1]oxa[4,7,10,14]tetraazacycloheptadecine-17-carboxamide |
| 6 | (7R,13S,17RS)-N-(2-(benzo[d][1,3]dioxol-5-yl)ethyl)-7-benzyl-11-(3-(benzyloxy)propyl)-13-isobutyl-14- |
| | methyl-9,12,15,19-tetraoxo-6,7,8,9,10,11,12,13,14,15,16,17,18,19-tetradecahydro- |
| | [1,3]dioxolo[4′,5′:4,5]benzo[1,2-p][1]oxa[4,7,10,14]tetraazacycloheptadecine-17-carboxamide |
| 7 | (7R,13S,17S)-N-(2-(benzo[d][1,3]dioxol-5-yl)ethyl)-7-benzyl-13-(cyclobutylmethyl)-11,14-dimethyl- |
| | 9,12,15,19-tetraoxo-6,7,8,9,10,11,12,13,14,15,16,17,18,19-tetradecahydro- |
| | [1,3]dioxolo[4′,5′:4,5]benzo[1,2-p][1]oxa[4,7,10,14]tetraazacycloheptadecine-17-carboxamide |
| 8 | (3R,9S,13S)-3-benzyl-9-isobutyl-17,18-dimethoxy-7,10-dimethyl-N-(2-(4-methyl-3,4-dihydro-2H- |
| | benzo[b][1,4]oxazin-7-yl)ethyl)-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 9 | (3R,9S,13RS)-N-(2-(benzo[d][1,3]dioxol-5-yl)ethyl)-3-benzyl-7-(2-cyclohexylethyl)-9- |
| | (cyclopropylmethyl)-17,18-dimethoxy-10-methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 10 | (3R,9S,13RS)-N-(2-(benzo[d][1,3]dioxol-5-yl)ethyl)-3-benzyl-7-(2-cyclohexylethyl)-9- |
| | (cyclopentylmethyl)-17,18-dimethoxy-10-methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 11 | (3R,9S,13RS)-3-benzyl-N-(2-(chroman-6-yl)ethyl)-7-(2-cyclohexylethyl)-9-isobutyl-16-methoxy-10- |
| | methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 12 | (3R,9S,13RS)-3-benzyl-7-(2-cyclohexylethyl)-N-(2-(3,4-dihydro-2H-benzo[b][1,4]dioxepin-7-yl)ethyl)-9- |
| | isobutyl-16-methoxy-10-methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 13 | (3R,9S,13RS)-3-benzyl-7-(2-cyclohexylethyl)-N-(2-(1,3-dihydroisobenzofuran-5-yl)ethyl)-9-isobutyl-16- |
| | methoxy-10-methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 14 | (3R,9S,13RS)-N-(2-(benzofuran-6-yl)ethyl)-3-benzyl-7-(2-cyclohexylethyl)-9-isobutyl-16-methoxy-10- |
| | methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 15 | (3R,9S,13RS)-3-benzyl-7-(2-cyclohexylethyl)-N-(2-(2,3-dihydrobenzofuran-6-yl)ethyl)-9-isobutyl-16- |
| | methoxy-10-methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 16 | (3R,9S,13RS)-N-(2-(benzo[d]oxazol-2-yl)ethyl)-3-benzyl-7-(2-cyclohexylethyl)-9-isobutyl-16-methoxy- |
| | 10-methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 17 | (8S,12S,18aR,21R)-21-benzyl-12-isobutyl-N-(3-methoxyphenethyl)-5,11-dimethyl-6,10,13,19-tetraoxo- |
| | 6,7,8,9,10,11,12,13,16,17,18,18a,19,20,21,22-hexadecahydro-5H,15H-imidazo[4′,5′:5,6]benzo[1,2- |
| | p]pyrido[2,1-f][1]oxa[4,7,10,14]tetraazacycloheptadecine-8-carboxamide |
| 18 | (3R,9S,13S)-3-benzyl-7-(2-cyclohexylethyl)-9-isobutyl-16-methoxy-10-methyl-5,8,11,15-tetraoxo-N-(2- |
| | (pyrazin-2-yl)ethyl)-2,3,4,5,6,7,8,9,10,11,12,13,14,15- |
| | tetradecahydrobenzo[p][1]oxa[4,7,10,14]tetraazacycloheptadecine-13-carboxamide |
| 19 | (3R,9S,13S)-3-benzyl-7-(2-cyclohexylethyl)-9-isobutyl-16-methoxy-10-methyl-5,8,11,15-tetraoxo-N-(2- |
| | (pyrimidin-4-yl)ethyl)-2,3,4,5,6,7,8,9,10,11,12,13,14,15- |
| | tetradecahydrobenzo[p][1]oxa[4,7,10,14]tetraazacycloheptadecine-13-carboxamide |
| 20 | (3R,9S,13S)-3-benzyl-7-(2-cyclohexylethyl)-9-isobutyl-16-methoxy-10-methyl-5,8,11,15-tetraoxo-N-(2- |
| | (pyrimidin-5-yl)ethyl)-2,3,4,5,6,7,8,9,10,11,12,13,14,15- |
| | tetradecahydrobenzo[p][1]oxa[4,7,10,14]tetraazacycloheptadecine-13-carboxamide |
| 21 | (3R,9S,13S)-3-benzyl-7-(2-cyclohexylethyl)-N-(3-(difluoromethoxy)propyl)-9-isobutyl-16-methoxy-10- |
| | methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 22 | (3R,9S,13S)-3-benzyl-7-(2-cyclohexylethyl)-9-isobutyl-16-methoxy-N-(2-(2-methoxyethoxy)ethyl)-10- |
| | methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 23 | (8RS,12S,18aR,21R)-21-benzyl-12-isobutyl-5,11-dimethyl-N-(2-(4-methylthiazol-2-yl)ethyl)-6,10,13,19- |
| | tetraoxo-6,7,8,9,10,11,12,13,16,17,18,18a,19,20,21,22-hexadecahydro-5H,15H- |
| | imidazo[4′,5′:5,6]benzo[1,2-p]pyrido[2,1-f][1]oxa[4,7,10,14]tetraazacycloheptadecine-8-carboxamide |
| 24 | (8RS,12S,18aR,21R)-21-benzyl-12-isobutyl-5,11-dimethyl-N-(2-(5-methylthiazol-2-yl)ethyl)-6,10,13,19- |
| | tetraoxo-6,7,8,9,10,11,12,13,16,17,18,18a,19,20,21,22-hexadecahydro-5H,15H- |
| | imidazo[4′,5′:5,6]benzo[1,2-p]pyrido[2,1-f][1]oxa[4,7,10,14]tetraazacycloheptadecine-8-carboxamide |
| 25 | (8RS,12S,18aR,21R)-21-benzyl-12-isobutyl-N-(4-methoxyphenethyl)-5,11-dimethyl-6,10,13,19- |
| | tetraoxo-6,7,8,9,10,11,12,13,16,17,18,18a,19,20,21,22-hexadecahydro-5H,15H- |
| | imidazo[4′,5′:5,6]benzo[1,2-p]pyrido[2,1-f][1]oxa[4,7,10,14]tetraazacycloheptadecine-8-carboxamide |
| 26 | (8RS,12S,18aR,21R)-21-benzyl-N-(3-(difluoromethoxy)phenethyl)-12-isobutyl-5,11-dimethyl- |
| | 6,10,13,19-tetraoxo-6,7,8,9,10,11,12,13,16,17,18,18a,19,20,21,22-hexadecahydro-5H,15H- |
| | imidazo[4′,5′:5,6]benzo[1,2-p]pyrido[2,1-f][1]oxa[4,7,10,14]tetraazacycloheptadecine-8-carboxamide |
| 27 | (8RS,12S,18aR,21R)-21-benzyl-12-isobutyl-5,11-dimethyl-6,10,13,19-tetraoxo-N-phenethyl- |
| | 6,7,8,9,10,11,12,13,16,17,18,18a,19,20,21,22-hexadecahydro-5H,15H-imidazo[4′,5′:5,6]benzo[1,2- |
| | p]pyrido[2,1-f][1]oxa[4,7,10,14]tetraazacycloheptadecine-8-carboxamide |
| 28 | (8RS,12S,18aR,21R)-N-(3-(2H-1,2,3-triazol-2-yl)phenethyl)-21-benzyl-12-isobutyl-5,11-dimethyl- |
| | 6,10,13,19-tetraoxo-6,7,8,9,10,11,12,13,16,17,18,18a,19,20,21,22-hexadecahydro-5H,15H- |
| | imidazo[4′,5′:5,6]benzo[1,2-p]pyrido[2,1-f][1]oxa[4,7,10,14]tetraazacycloheptadecine-8-carboxamide |
| 29 | (8S,12S,18aR,21R)-21-benzyl-N-(2-(5,6-dihydro-4H-cyclopenta[d]thiazol-2-yl)ethyl)-12-isobutyl-5,11- |
| | dimethyl-6,10,13,19-tetraoxo-6,7,8,9,10,11,12,13,16,17,18,18a,19,20,21,22-hexadecahydro-5H,15H- |
| | imidazo[4′,5′:5,6]benzo[1,2-p]pyrido[2,1-f][1]oxa[4,7,10,14]tetraazacycloheptadecine-8-carboxamide |
| 30 | (8S,12S,18aR,21R)-N-(2-(benzo[d]isoxazol-3-yl)ethyl)-21-benzyl-12-isobutyl-5,11-dimethyl-6,10,13,19- |
| | tetraoxo-6,7,8,9,10,11,12,13,16,17,18,18a,19,20,21,22-hexadecahydro-5H,15H- |
| | imidazo[4′,5′:5,6]benzo[1,2-p]pyrido[2,1-f][1]oxa[4,7,10,14]tetraazacycloheptadecine-8-carboxamide |
| 31 | (8S,12S,18aR,21R)-N-(4-(2H-1,2,3-triazol-2-yl)phenethyl)-21-benzyl-12-isobutyl-5,11-dimethyl- |
| | 6,10,13,19-tetraoxo-6,7,8,9,10,11,12,13,16,17,18,18a,19,20,21,22-hexadecahydro-5H,15H- |
| | imidazo[4′,5′:5,6]benzo[1,2-p]pyrido[2,1-f][1]oxa[4,7,10,14]tetraazacycloheptadecine-8-carboxamide |
| 32 | (4aR,7R,18S,22S)-7-benzyl-22-isobutyl-N-(-8-methoxychroman-3-yl)-21-methyl-5,16,20,23-tetraoxo- |
| | 1,2,4,4a,5,6,7,8,16,17,18,19,20,21,22,23-hexadecahydronaphtho[1,2-p][1,4]oxazino[3,4- |
| | f][1]oxa[4,7,10,14]tetraazacycloheptadecine-18-carboxamide |
| 33 | (4aR,7R,18S,22S)-7-benzyl-22-isobutyl-N-(2-(3-methoxyphenoxy)ethyl)-21-methyl-5,16,20,23-tetraoxo- |
| | 1,2,4,4a,5,6,7,8,16,17,18,19,20,21,22,23-hexadecahydronaphtho[1,2-p][1,4]oxazino[3,4- |
| | f][1]oxa[4,7,10,14]tetraazacycloheptadecine-18-carboxamide |
| 34 | (4aR,7R,18S,22S)-7-benzyl-22-isobutyl-N-(2-(4-methoxyphenoxy)ethyl)-21-methyl-5,16,20,23-tetraoxo- |
| | 1,2,4,4a,5,6,7,8,16,17,18,19,20,21,22,23-hexadecahydronaphtho[1,2-p][1,4]oxazino[3,4- |
| | f][1]oxa[4,7,10,14]tetraazacycloheptadecine-18-carboxamide |
| 35 | (4aR,7R,18S,22S)-7-benzyl-22-isobutyl-N-(2-(7-methoxy-2,3-dihydrobenzo[b][1,4]dioxin-6-yl)ethyl)-21- |
| | methyl-5,16,20,23-tetraoxo-1,2,4,4a,5,6,7,8,16,17,18,19,20,21,22,23-hexadecahydronaphtho[1,2- |
| | p][1,4]oxazino[3,4-f][1]oxa[4,7,10,14]tetraazacycloheptadecine-18-carboxamide |
| 36 | (4aR,7R,18S,22S)-7-benzyl-N-(2-(7-chloro-5-methyl-2,3-dihydrobenzo[b][1,4]dioxin-6-yl)ethyl)-22- |
| | isobutyl-21-methyl-5,16,20,23-tetraoxo-1,2,4,4a,5,6,7,8,16,17,18,19,20,21,22,23- |
| | hexadecahydronaphtho[1,2-p][1,4]oxazino[3,4-f][1]oxa[4,7,10,14]tetraazacycloheptadecine-18- |
| | carboxamide |
| 37 | (4aR,7R,18S,22S)-7-benzyl-N-(2-(7-chloro-2,3-dihydrobenzo[b][1,4]dioxin-6-yl)ethyl)-22-isobutyl-21- |
| | methyl-5,16,20,23-tetraoxo-1,2,4,4a,5,6,7,8,16,17,18,19,20,21,22,23-hexadecahydronaphtho[1,2- |
| | p][1,4]oxazino[3,4-f][1]oxa[4,7,10,14]tetraazacycloheptadecine-18-carboxamide |
| 38 | (4aR,7R,18S,22S)-N-(2-(benzo[d]isoxazol-3-yl)ethyl)-7-benzyl-22-isobutyl-21-methyl-5,16,20,23- |
| | tetraoxo-1,2,4,4a,5,6,7,8,16,17,18,19,20,21,22,23-hexadecahydronaphtho[1,2-p][1,4]oxazino[3,4- |
| | f][1]oxa[4,7,10,14]tetraazacycloheptadecine-18-carboxamide |
| 39 | (4aR,7R,18S,22S)-7-benzyl-N-(2-(2,3-dihydrobenzo[b][1,4]dioxin-6-yl)ethyl)-22-isobutyl-21-methyl- |
| | 5,16,20,23-tetraoxo-1,2,4,4a,5,6,7,8,16,17,18,19,20,21,22,23-hexadecahydronaphtho[1,2- |
| | p][1,4]oxazino[3,4-f][1]oxa[4,7,10,14]tetraazacycloheptadecine-18-carboxamide |
| 40 | (4aR,7R,18S,22S)-7-benzyl-N-(4-cyanophenethyl)-22-isobutyl-21-methyl-5,16,20,23-tetraoxo- |
| | 1,2,4,4a,5,6,7,8,16,17,18,19,20,21,22,23-hexadecahydronaphtho[1,2-p][1,4]oxazino[3,4- |
| | f][1]oxa[4,7,10,14]tetraazacycloheptadecine-18-carboxamide |
| 41 | (4aR,7R,18S,22S)-N-(3-(2H-1,2,3-triazol-2-yl)phenethyl)-7-benzyl-22-isobutyl-21-methyl-5,16,20,23- |
| | tetraoxo-1,2,4,4a,5,6,7,8,16,17,18,19,20,21,22,23-hexadecahydronaphtho[1,2-p][1,4]oxazino[3,4- |
| | f][1]oxa[4,7,10,14]tetraazacycloheptadecine-18-carboxamide |
| 42 | (4aR,7R,18S,22S)-7-benzyl-N-(2-(2,3-dihydrobenzofuran-6-yl)ethyl)-22-isobutyl-21-methyl-5,16,20,23- |
| | tetraoxo-1,2,4,4a,5,6,7,8,16,17,18,19,20,21,22,23-hexadecahydronaphtho[1,2-p][1,4]oxazino[3,4- |
| | f][1]oxa[4,7,10,14]tetraazacycloheptadecine-18-carboxamide |
| 43 | (4aR,7R,18S,22S)-7-benzyl-N-(2-(3,5-dimethylisoxazol-4-yl)ethyl)-22-isobutyl-21-methyl-5,16,20,23- |
| | tetraoxo-1,2,4,4a,5,6,7,8,16,17,18,19,20,21,22,23-hexadecahydronaphtho[1,2-p][1,4]oxazino[3,4- |
| | f][1]oxa[4,7,10,14]tetraazacycloheptadecine-18-carboxamide |
| 44 | (4aR,7R,18S,22S)-7-benzyl-N-(2-(5-ethyl-1,2,4-oxadiazol-3-yl)ethyl)-22-isobutyl-21-methyl-5,16,20,23- |
| | tetraoxo-1,2,4,4a,5,6,7,8,16,17,18,19,20,21,22,23-hexadecahydronaphtho[1,2-p][1,4]oxazino[3,4- |
| | f][1]oxa[4,7,10,14]tetraazacycloheptadecine-18-carboxamide |
| 45 | (4aR,7R,18S,22S)-7-benzyl-N-(2-(5-cyclopropyl-1,2,4-oxadiazol-3-yl)ethyl)-22-isobutyl-21-methyl- |
| | 5,16,20,23-tetraoxo-1,2,4,4a,5,6,7,8,16,17,18,19,20,21,22,23-hexadecahydronaphtho[1,2- |
| | p][1,4]oxazino[3,4-f][1]oxa[4,7,10,14]tetraazacycloheptadecine-18-carboxamide |
| 46 | (9S,13S,19aR,22R)-22-benzyl-13-isobutyl-N-(2-(2-isopropylthiazol-4-yl)ethyl)-12-methyl-7,11,14,20- |
| | tetraoxo-7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 47 | (9S,13S,19aR,22R)-22-benzyl-N-(4-ethylphenethyl)-13-isobutyl-12-methyl-7,11,14,20-tetraoxo- |
| | 7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 48 | (9S,13S,19aR,22R)-22-benzyl-N-(4-(tert-butyl)phenethyl)-13-isobutyl-12-methyl-7,11,14,20-tetraoxo- |
| | 7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 49 | (9S,13S,19aR,22R)-22-benzyl-N-(4-bromophenethyl)-13-isobutyl-12-methyl-7,11,14,20-tetraoxo- |
| | 7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 50 | (9S,13S,19aR,22R)-22-benzyl-N-(2-fluorophenethyl)-13-isobutyl-12-methyl-7,11,14,20-tetraoxo- |
| | 7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 51 | (9S,13S,19aR,22R)-22-benzyl-N-(3-chlorophenethyl)-13-isobutyl-12-methyl-7,11,14,20-tetraoxo- |
| | 7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 52 | (9S,13S,19aR,22R)-22-benzyl-N-(3-bromophenethyl)-13-isobutyl-12-methyl-7,11,14,20-tetraoxo- |
| | 7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 53 | (9S,13S,19aR,22R)-22-benzyl-N-(2-(2,3-dihydrobenzo[b][1,4]dioxin-6-yl)ethyl)-13-isobutyl-12-methyl- |
| | 7,11,14,20-tetraoxo-7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 54 | (9S,13S,19aR,22R)-22-benzyl-N-(2-(2,3-dihydrobenzofuran-6-yl)ethyl)-13-isobutyl-12-methyl- |
| | 7,11,14,20-tetraoxo-7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 55 | (9S,13S,19aR,22R)-N-(4-(2H-1,2,3-triazol-2-yl)phenethyl)-22-benzyl-13-isobutyl-12-methyl-7,11,14,20- |
| | tetraoxo-7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 56 | (9S,13S,19aR,22R)-N-(3-(2H-1,2,3-triazol-2-yl)phenethyl)-22-benzyl-13-isobutyl-12-methyl-7,11,14,20- |
| | tetraoxo-7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 57 | (9S,13S,19aR,22R)-22-benzyl-N-(3,5-dimethoxyphenethyl)-13-isobutyl-12-methyl-7,11,14,20-tetraoxo- |
| | 7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 58 | (9S,13S,19aR,22R)-22-benzyl-13-isobutyl-12-methyl-7,11,14,20-tetraoxo-N-phenethyl- |
| | 7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 59 | (9S,13S,19aR,22R)-22-benzyl-N-(2-fluoro-4-methoxyphenethyl)-13-isobutyl-12-methyl-7,11,14,20- |
| | tetraoxo-7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 60 | (9S,13S,19aR,22R)-22-benzyl-13-isobutyl-12-methyl-N-(4-methylphenethyl)-7,11,14,20-tetraoxo- |
| | 7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 61 | (9S,13S,19aR,22R)-22-benzyl-N-(3,4-dimethylphenethyl)-13-isobutyl-12-methyl-7,11,14,20-tetraoxo- |
| | 7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 62 | (9S,13S,19aR,22R)-22-benzyl-13-isobutyl-12-methyl-N-(3-methylphenethyl)-7,11,14,20-tetraoxo- |
| | 7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 63 | (9S,13S,19aR,22R)-22-benzyl-N-(2,4-dimethylphenethyl)-13-isobutyl-12-methyl-7,11,14,20-tetraoxo- |
| | 7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 64 | (9S,13S,19aR,22R)-22-benzyl-13-isobutyl-12-methyl-N-(2-methylphenethyl)-7,11,14,20-tetraoxo- |
| | 7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 65 | (9S,13S,19aR,22R)-22-benzyl-N-(2,3-dimethoxyphenethyl)-13-isobutyl-12-methyl-7,11,14,20-tetraoxo- |
| | 7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 66 | (9S,13S,19aR,22R)-22-benzyl-N-(3-fluorophenethyl)-13-isobutyl-12-methyl-7,11,14,20-tetraoxo- |
| | 7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 67 | (9S,13S,19aR,22R)-22-benzyl-N-(4-fluorophenethyl)-13-isobutyl-12-methyl-7,11,14,20-tetraoxo- |
| | 7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 68 | (9S,13S,19aR,22R)-22-benzyl-N-(2,6-difluorophenethyl)-13-isobutyl-12-methyl-7,11,14,20-tetraoxo- |
| | 7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 69 | (9S,13S,19aR,22R)-22-benzyl-13-isobutyl-12-methyl-7,11,14,20-tetraoxo-N-(2-(3-phenyl-1,2,4- |
| | oxadiazol-5-yl)ethyl)-7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 70 | (4aR,7R,18S,22S)-7-benzyl-N-(2-(4,5-dimethylisoxazol-3-yl)ethyl)-22-isobutyl-21-methyl-5,16,20,23- |
| | tetraoxo-1,2,4,4a,5,6,7,8,16,17,18,19,20,21,22,23-hexadecahydronaphtho[1,2-p][1,4]oxazino[3,4- |
| | f][1]oxa[4,7,10,14]tetraazacycloheptadecine-18-carboxamide |
| 71 | (9S,13S,19aR,22R)-22-benzyl-N-(3,5-dimethoxyphenethyl)-13-isobutyl-12-methyl-7,11,14,20-tetraoxo- |
| | 7,8,9,10,11,12,13,14,17,18,19,19a,20,21,22,23-hexadecahydro-16H- |
| | pyrido[2′,11:6,7][1]oxa[4,7,10,14]tetraazacycloheptadecino[17,16-c]quinoline-9-carboxamide |
| 72 | (9S,13S,19aR,22R)-22-benzyl-N-(2,6-difluorophenethyl)-13-isobutyl-12-methyl-7,11,14,20-tetraoxo- |
| | 7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 73 | (9S,13S,19aR,22R)-22-benzyl-13-isobutyl-12-methyl-7,11,14,20-tetraoxo-N-(2,4,6-trifluorophenethyl)- |
| | 7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 74 | (9S,13S,19aR,22R)-22-benzyl-N-(2,6-difluoro-4-methoxyphenethyl)-13-isobutyl-12-methyl-7,11,14,20- |
| | tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 75 | (9S,13S,19aR,22R)-22-benzyl-N-(3,4-difluorophenethyl)-13-isobutyl-12-methyl-7,11,14,20-tetraoxo- |
| | 7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 76 | (9S,13S,19aR,22R)-22-benzyl-13-isobutyl-12-methyl-7,11,14,20-tetraoxo-N-(2-(3-phenyl-1,2,4- |
| | oxadiazol-5-yl)ethyl)-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 77 | (9S,13S,19aR,22R)-22-benzyl-13-isobutyl-12-methyl-7,11,14,20-tetraoxo-N-phenethyl- |
| | 7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 78 | (9S,13S,19aR,22R)-22-benzyl-13-isobutyl-12-methyl-N-(2-(5-methyl-1,2,4-oxadiazol-3-yl)ethyl)- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 79 | (9S,13S,19aR,22R)-22-benzyl-N-(2-(5-ethyl-1,2,4-oxadiazol-3-yl)ethyl)-13-isobutyl-12-methyl- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 80 | (9S,13S,19aR,22R)-22-benzyl-N-(2-(5-cyclopropyl-1,2,4-oxadiazol-3-yl)ethyl)-13-isobutyl-12-methyl- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 81 | (9S,13S,19aR,22R)-22-benzyl-N-(2,3-dimethoxyphenethyl)-13-isobutyl-12-methyl-7,11,14,20-tetraoxo- |
| | 7,8,9,10,11,12,13,14,17,18,19,19a,20,21,22,23-hexadecahydro-16H- |
| | pyrido[2′,11:6,7][1]oxa[4,7,10,14]tetraazacycloheptadecino[17,16-c]quinoline-9-carboxamide |
| 82 | (9S,13S,19aR,22R)-22-benzyl-13-isobutyl-12-methyl-N-(2-(5-methylisoxazol-4-yl)ethyl)-7,11,14,20- |
| | tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 83 | (9S,13S,19aR,22R)-22-benzyl-13-isobutyl-12-methyl-N-(2-(1-methyl-1H-pyrazol-4-yl)ethyl)-7,11,14,20- |
| | tetraoxo-7,8,9,10,11,12,13,14,17,18,19,19a,20,21,22,23-hexadecahydro-16H- |
| | pyrido[2′,11:6,7][1]oxa[4,7,10,14]tetraazacycloheptadecino[17,16-c]quinoline-9-carboxamide |
| 84 | (9S,13S,19aR,22R)-22-benzyl-13-isobutyl-N-(4-methoxyphenethyl)-12-methyl-7,11,14,20-tetraoxo- |
| | 7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 85 | (9S,13S,19aR,22R)-22-benzyl-N-(2-(5-(tert-butyl)-1,2,4-oxadiazol-3-yl)ethyl)-13-isobutyl-12-methyl- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 86 | (9S,13S,19aR,22R)-22-benzyl-N-(2-(3-(3,5-dimethylphenyl)-1,2,4-oxadiazol-5-yl)ethyl)-13-isobutyl-12- |
| | methyl-7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 87 | (9S,13S,19aR,22R)-22-benzyl-13-isobutyl-12-methyl-7,11,14,20-tetraoxo-N-(2-(3-(2- |
| | (trifluoromethoxy)phenyl)-1,2,4-oxadiazol-5-yl)ethyl)-7,8,9,10,11,12,13,14,17,18,19,19a,20,21,22,23- |
| | hexadecahydro-16H-pyrido[2′,1′:6,7][1]oxa[4,7,10,14]tetraazacycloheptadecino[17,16-c]quinoline-9- |
| | carboxamide |
| 88 | (9S,13S,19aR,22R)-22-benzyl-13-isobutyl-12-methyl-7,11,14,20-tetraoxo-N-(2-(5-(2- |
| | (trifluoromethoxy)phenyl)-4H-1,2,4-triazol-3-yl)ethyl)-7,8,9,10,11,12,13,14,17,18,19,19a,20,21,22,23- |
| | hexadecahydro-16H-pyrido[2′,1′:6,7][1]oxa[4,7,10,14]tetraazacycloheptadecino[17,16-c]quinoline-9- |
| | carboxamide |
| 89 | (9S,13S,19aR,22R)-22-benzyl-N-(2-(5-(tert-butyl)isoxazol-3-yl)ethyl)-13-isobutyl-12-methyl-7,11,14,20- |
| | tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 90 | (9S,13S,19aS,22R)-22-benzyl-N-(2-(5-(tert-butyl)isoxazol-3-yl)ethyl)-13-isobutyl-12-methyl-7,11,14,20- |
| | tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 91 | (9S,13S,19aR,22R)-22-benzyl-N-(4-fluoro-3-methoxyphenethyl)-13-isobutyl-12-methyl-7,11,14,20- |
| | tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 92 | (9S,13S,19aR,22R)-22-benzyl-N-(2-fluoro-5-methylphenethyl)-13-isobutyl-12-methyl-7,11,14,20- |
| | tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 93 | (9S,13S,19aR,22R)-22-benzyl-13-isobutyl-N-(3-methoxy-4-(2H-1,2,3-triazol-2-yl)phenethyl)-12-methyl- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 94 | (9S,13S,19aR,22R)-22-benzyl-N-(2,4-difluorophenethyl)-13-isobutyl-12-methyl-7,11,14,20-tetraoxo- |
| | 7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 95 | (9S,13S,19aR,22R)-22-benzyl-13-isobutyl-N-(4-methoxy-3-methylphenethyl)-12-methyl-7,11,14,20- |
| | tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 96 | (9S,13S,19aR,22R)-22-benzyl-13-isobutyl-12-methyl-N-(3-methylphenethyl)-7,11,14,20-tetraoxo- |
| | 7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 97 | (9S,13S,19aR,22R)-22-benzyl-N-(2,3-difluorophenethyl)-13-isobutyl-12-methyl-7,11,14,20-tetraoxo- |
| | 7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 98 | (9S,13S,19aR,22R)-22-benzyl-13-isobutyl-12-methyl-N-(2-(5-methylisoxazol-3-yl)ethyl)-7,11,14,20- |
| | tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 99 | (9S,13S,19aR,22R)-22-benzyl-N-(4-bromo-2,6-difluorophenethyl)-13-isobutyl-12-methyl-7,11,14,20- |
| | tetraoxo-7,8,9,10,11,12,13,14,17,18,19,19a,20,21,22,23-hexadecahydro-16H- |
| | pyrido[2′,11:6,7][1]oxa[4,7,10,14]tetraazacycloheptadecino[17,16-c]quinoline-9-carboxamide |
| 100 | (9S,13S,19aR,22R)-22-benzyl-N-(3-fluoro-4-methoxyphenethyl)-13-isobutyl-12-methyl-7,11,14,20- |
| | tetraoxo-7,8,9,10,11,12,13,14,17,18,19,19a,20,21,22,23-hexadecahydro-16H- |
| | pyrido[2′,11:6,7][1]oxa[4,7,10,14]tetraazacycloheptadecino[17,16-c]quinoline-9-carboxamide |
| 101 | (7R,13S,17S)-7-benzyl-N-(2-(3,4-dihydro-2H-benzo[b][1,4]dioxepin-7-yl)ethyl)-13-isobutyl-11,14- |
| | dimethyl-9,12,15,19-tetraoxo-6,7,8,9,10,11,12,13,14,15,16,17,18,19-tetradecahydro- |
| | [1,3]dioxolo[4′,5′:4,5]benzo[1,2-p][1]oxa[4,7,10,14]tetraazacycloheptadecine-17-carboxamide |
| 102 | (9S,13S,19aR,22R)-22-benzyl-N-(2,6-difluoro-3-methylphenethyl)-13-isobutyl-12-methyl-7,11,14,20- |
| | tetraoxo-7,8,9,10,11,12,13,14,17,18,19,19a,20,21,22,23-hexadecahydro-16H- |
| | pyrido[2′,11:6,7][1]oxa[4,7,10,14]tetraazacycloheptadecino[17,16-c]quinoline-9-carboxamide |
| 103 | (9S,13S,19aR,22R)-22-benzyl-13-isobutyl-N-(2-(isothiazol-5-yl)ethyl)-12-methyl-7,11,14,20-tetraoxo- |
| | 7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 104 | (9S,13S,19aR,22R)-22-benzyl-N-(2-fluoro-6-methylphenethyl)-13-isobutyl-12-methyl-7,11,14,20- |
| | tetraoxo-7,8,9,10,11,12,13,14,17,18,19,19a,20,21,22,23-hexadecahydro-16H- |
| | pyrido[2′,11:6,7][1]oxa[4,7,10,14]tetraazacycloheptadecino[17,16-c]quinoline-9-carboxamide |
| 105 | (9S,13S,19aR,22R)-22-benzyl-N-(2-(5-cyclopropylisoxazol-3-yl)ethyl)-13-isobutyl-12-methyl-7,11,14,20- |
| | tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 106 | (9S,13S,19aR,22R)-22-benzyl-N-(2-(5-cyclopropylisoxazol-3-yl)ethyl)-13-isobutyl-12-methyl-7,11,14,20- |
| | tetraoxo-7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 107 | (9S,13S,19aR,22R)-22-benzyl-N-(2-(5-(tert-butyl)isoxazol-3-yl)ethyl)-13-isobutyl-12-methyl-7,11,14,20- |
| | tetraoxo-7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 108 | (3′S,7′S,13′R)-13′-benzyl-7′-isobutyl-6′,9′-dimethyl-1′,5′,8′,11′-tetraoxo-N-phenethyl- |
| | 2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 109 | (3′S,7′S,13′R)-13′-benzyl-N-(3,5-dimethoxyphenethyl)-7′-isobutyl-6′,9′-dimethyl-1′,5′,8′,11′-tetraoxo- |
| | 2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 110 | (3′S,7′S,13′R)-13′-benzyl-N-(2,6-difluorophenethyl)-7′-isobutyl-6′,9′-dimethyl-1′,5′,8′,11′-tetraoxo- |
| | 2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 111 | (3′S,7′S,13′R)-13′-benzyl-7′-isobutyl-N-(4-methoxyphenethyl)-6′,9′-dimethyl-1′,5′,8′,11′-tetraoxo- |
| | 2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 112 | (3′S,7′S,13′R)-13′-benzyl-7′-isobutyl-6′,9′-dimethyl-N-(3-methylphenethyl)-1′,5′,8′,11′-tetraoxo- |
| | 2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 113 | (3′S,7′S,13′R)-13′-benzyl-N-(3,4-difluorophenethyl)-7′-isobutyl-6′,9′-dimethyl-1′,5′,8′,11′-tetraoxo- |
| | 2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 114 | (3′S,7′S,13′R)-13′-benzyl-N-(2-(5-ethyl-1,2,4-oxadiazol-3-yl)ethyl)-7′-isobutyl-6′,9′-dimethyl-1′,5′,8′,11′- |
| | tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 115 | (3′S,7′S,13′R)-13′-benzyl-N-(2-(5-cyclopropyl-1,2,4-oxadiazol-3-yl)ethyl)-7′-isobutyl-6′,9′-dimethyl- |
| | 1′,5′,8′,11′-tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 116 | (3′S,7′S,13′R)-13′-benzyl-N-(2-(5-(tert-butyl)-1,2,4-oxadiazol-3-yl)ethyl)-7′-isobutyl-6′,9′-dimethyl- |
| | 1′,5′,8′,11′-tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 117 | (3′S,7′S,13′R)-13′-benzyl-7′-isobutyl-6′,9′-dimethyl-N-(2-(5-methylisoxazol-3-yl)ethyl)-1′,5′,8′,11′- |
| | tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 118 | (3′S,7′S,13′R)-13′-benzyl-N-(2-(5-cyclopropylisoxazol-3-yl)ethyl)-7′-isobutyl-6′,9′-dimethyl-1′,5′,8′,11′- |
| | tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 119 | (3′S,7′S,13′R)-13′-benzyl-N-(2-(5-(tert-butyl)isoxazol-3-yl)ethyl)-7′-isobutyl-6′,9′-dimethyl-1′,5′,8′,11′- |
| | tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 120 | (3′S,7′S,13′R)-13′-benzyl-N-(3,5-difluorophenethyl)-7′-isobutyl-6′,9′-dimethyl-1′,5′,8′,11′-tetraoxo- |
| | 2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 121 | (3′S,7′S,13′R)-13′-benzyl-N-(2-(3-cyclopropyl-1,2,4-oxadiazol-5-yl)ethyl)-7′-isobutyl-6′,9′-dimethyl- |
| | 1′,5′,8′,11′-tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 122 | (9S,13S,19aR,22R)-22-benzyl-13-isobutyl-12-methyl-7,11,14,20-tetraoxo-N-(4-(pyrimidin-2- |
| | yl)phenethyl)-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 123 | (9S,13S,19aR,22R)-22-benzyl-13-isobutyl-12-methyl-7,11,14,20-tetraoxo-N-(3-(pyrimidin-2- |
| | yl)phenethyl)-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 124 | (9S,13S,19aR,22R)-22-benzyl-N-(2-(1-cyclopropyl-1H-pyrazol-4-yl)ethyl)-13-isobutyl-12-methyl- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 125 | (9S,13S,19aR,22R)-22-benzyl-13-isobutyl-12-methyl-N-(3-methyl-4-(2H-1,2,3-triazol-2-yl)phenethyl)- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 126 | (9S,13S,19aR,22R)-22-benzyl-N-(3,5-difluorophenethyl)-13-isobutyl-12-methyl-7,11,14,20-tetraoxo- |
| | 7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 127 | (9S,13S,19aR,22R)-22-benzyl-N-(2-(3-cyclopropyl-1,2,4-oxadiazol-5-yl)ethyl)-13-isobutyl-12-methyl- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 128 | (9S,13S,19aR,22R)-22-benzyl-N-(2-(5-cyclobutyl-1,2,4-oxadiazol-3-yl)ethyl)-13-isobutyl-12-methyl- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 129 | (9S,13S,19aR,22R)-22-benzyl-N-(2-(1,4-dimethyl-1H-pyrazol-3-yl)ethyl)-13-isobutyl-12-methyl- |
| | 7,11,14,20-tetraoxo-7,8,9,10,11,12,13,14,17,18,19,19a,20,21,22,23-hexadecahydro-16H- |
| | pyrido[2′,11:6,7][1]oxa[4,7,10,14]tetraazacycloheptadecino[17,16-c]quinoline-9-carboxamide |
| 130 | (9S,13S,19aR,22R)-22-benzyl-13-isobutyl-N-(2-(3-(3-methoxyphenyl)-1,2,4-oxadiazol-5-yl)ethyl)-12- |
| | methyl-7,11,14,20-tetraoxo-7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 131 | (3′S,7′S,13′R)-13′-benzyl-20′-fluoro-7′-isobutyl-N-(3-methoxyphenethyl)-6′,9′-dimethyl-1′,5′,8′,11′- |
| | tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 132 | (3′S,7′S,13′R)-13′-benzyl-N-(2,6-difluorophenethyl)-20′-fluoro-7′-isobutyl-6′,9′-dimethyl-1′,5′,8′,11′- |
| | tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 133 | (3′S,7′S,13′R)-13′-benzyl-20′-fluoro-7′-isobutyl-6′,9′-dimethyl-1′,5′,8′,11′-tetraoxo-N-(4-(pyrazin-2- |
| | yl)phenethyl)-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 134 | (3′S,7′S,13′R)-13′-benzyl-20′-fluoro-7′-isobutyl-6′,9′-dimethyl-1′,5′,8′,11′-tetraoxo-N-(3-(pyrazin-2- |
| | yl)phenethyl)-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 135 | (3′S,7′S,13′R)-N-(4-(2H-1,2,3-triazol-2-yl)phenethyl)-13′-benzyl-20′-fluoro-7′-isobutyl-6′,9′-dimethyl- |
| | 1′,5′,8′,11′-tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 136 | (3′S,7′S,13′R)-N-(3-(2H-1,2,3-triazol-2-yl)phenethyl)-13′-benzyl-20′-fluoro-7′-isobutyl-6′,9′-dimethyl- |
| | 1′,5′,8′,11′-tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 137 | (3′S,7′S,13′R)-13′-benzyl-20′-fluoro-7′-isobutyl-6′,9′-dimethyl-1′,5′,8′,11′-tetraoxo-N-(4-(pyrimidin-2- |
| | yl)phenethyl)-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 138 | (3′S,7′S,13′R)-13′-benzyl-20′-fluoro-7′-isobutyl-6′,9′-dimethyl-1′,5′,8′,11′-tetraoxo-N-(3-(pyrimidin-2- |
| | yl)phenethyl)-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 139 | (3′S,7′S,13′R)-13′-benzyl-N-(2-(1-cyclopropyl-1H-pyrazol-4-yl)ethyl)-20′-fluoro-7′-isobutyl-6′,9′-dimethyl- |
| | 1′,5′,8′,11′-tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 140 | (3′S,7′S,13′R)-13′-benzyl-N-(2-(3-cyclopropyl-1,2,4-oxadiazol-5-yl)ethyl)-20′-fluoro-7′-isobutyl-6′,9′- |
| | dimethyl-1′,5′,8′,11′-tetraoxo-2′,3′,4′,5′,6′,7′,8′,9′,11′,12′,13′,14′-dodecahydro-1′H-spiro[cyclopropane- |
| | 1,10′-[1]oxa[4,7,10,14]tetraazacycloheptadecino[17,16-c]quinoline]-3′-carboxamide |
| 141 | (3′S,7′S,13′R)-13′-benzyl-20′-fluoro-7′-isobutyl-6′,9′-dimethyl-N-(2-(5-methylisoxazol-3-yl)ethyl)- |
| | 1′,5′,8′,11′-tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 142 | (3′S,7′S,13′R)-13′-benzyl-N-(2-(5-cyclopropylisoxazol-3-yl)ethyl)-20′-fluoro-7′-isobutyl-6′,9′-dimethyl- |
| | 1′,5′,8′,11′-tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 143 | (3′S,7′S,13′R)-13′-benzyl-20′-fluoro-7′-isobutyl-6′,9′-dimethyl-N-(2-(1-methyl-1H-pyrazol-4-yl)ethyl)- |
| | 1′,5′,8′,11′-tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 144 | (3′S,7′S,13′R)-13′-benzyl-N-(2-(5-cyclopropyl-1,2,4-oxadiazol-3-yl)ethyl)-20′-fluoro-7′-isobutyl-6′,9′- |
| | dimethyl-1′,5′,8′,11′-tetraoxo-2′,3′,4′,5′,6′,7′,8′,9′,11′,12′,13′,14′-dodecahydro-1′H-spiro[cyclopropane- |
| | 1,10′-[1]oxa[4,7,10,14]tetraazacycloheptadecino[17,16-c]quinoline]-3′-carboxamide |
| 145 | (3′S,7′S,13′R)-13′-benzyl-N-(2,2-difluoro-2-phenylethyl)-7′-isobutyl-6′,9′-dimethyl-1′,5′,8′,11′-tetraoxo- |
| | 2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 146 | (3′S,7′S,13′R)-13′-benzyl-20′-fluoro-7′-isobutyl-6′,9′-dimethyl-N-(2-(3-methyl-1,2,4-oxadiazol-5-yl)ethyl)- |
| | 1′,5′,8′,11′-tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 147 | (3′S,7′S,13′R)-13′-benzyl-20′-fluoro-7′-isobutyl-6′,9′-dimethyl-N-(2-(3-methylisoxazol-5-yl)ethyl)- |
| | 1′,5′,8′,11′-tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 148 | (3R,9S,13S)-3-benzyl-N-(2-(chroman-7-yl)ethyl)-9-isobutyl-17,18-dimethoxy-7,10-dimethyl-5,8,11,15- |
| | tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 149 | (3R,9S,13S)-3-benzyl-7-(2-cyclohexylethyl)-N-(2-(2,3-dihydrobenzo[b][1,4]dioxin-6-yl)ethyl)-9-isobutyl- |
| | 16-methoxy-10-methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 150 | (3R,9S,13S)-3-benzyl-7-(2-cyclohexylethyl)-N-(3-(difluoromethoxy)phenethyl)-9-isobutyl-16-methoxy- |
| | 10-methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 151 | (7R,13S,17RS)-7-benzyl-N-(2,3-dimethoxyphenethyl)-13-isobutyl-11,14-dimethyl-9,12,15,19-tetraoxo- |
| | 6,7,8,9,10,11,12,13,14,15,16,17,18,19-tetradecahydro-[1,3]dioxolo[4′,5′:4,5]benzo[1,2- |
| | p][1]oxa[4,7,10,14]tetraazacycloheptadecine-17-carboxamide |
| 152 | (3R,9S,13RS)-3-benzyl-N-(2-(2,3-dihydrobenzofuran-5-yl)ethyl)-9-isobutyl-17,18-dimethoxy-7,10- |
| | dimethyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 153 | (7R,13S,17S)-7-benzyl-13-isobutyl-N-(4-methoxyphenethyl)-11,14-dimethyl-9,12,15,19-tetraoxo- |
| | 6,7,8,9,10,11,12,13,14,15,16,17,18,19-tetradecahydro-[1,3]dioxolo[4′,5′:4,5]benzo[1,2- |
| | p][1]oxa[4,7,10,14]tetraazacycloheptadecine-17-carboxamide |
| 154 | (7R,13S,17S)-N-(2-(benzo[d][1,3]dioxol-5-yl)ethyl)-7-benzyl-13-isobutyl-14-methyl-9,12,15,19-tetraoxo- |
| | 11-(2-(tetrahydro-2H-pyran-4-yl)ethyl)-6,7,8,9,10,11,12,13,14,15,16,17,18,19-tetradecahydro- |
| | [1,3]dioxolo[4′,5′:4,5]benzo[1,2-p][1]oxa[4,7,10,14]tetraazacycloheptadecine-17-carboxamide |
| 155 | (3R,9S,13S)-N-(2-(benzo[d][1,3]dioxol-5-yl)ethyl)-3-benzyl-9-isobutyl-17,18-dimethoxy-10-methyl- |
| | 5,8,11,15-tetraoxo-7-(2-((RS)-tetrahydro-2H-pyran-2-yl)ethyl)-2,3,4,5,6,7,8,9,10,11,12,13,14,15- |
| | tetradecahydrobenzo[p][1]oxa[4,7,10,14]tetraazacycloheptadecine-13-carboxamide |
| 156 | (3R,9S,13RS)-N-(2-(benzo[d][1,3]dioxol-5-yl)ethyl)-3-benzyl-9-isobutyl-17,18-dimethoxy-7-(3-methoxy- |
| | 3-methylbutyl)-10-methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 157 | (3R,9S,13S)-3-benzyl-7-(2-cyclohexylethyl)-N-((RS)-2-(5,5-dimethyltetrahydro-2H-pyran-2-yl)ethyl)-9- |
| | isobutyl-16-methoxy-10-methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 158 | (3R,9S,13S)-3-benzyl-7-(2-cyclohexylethyl)-9-isobutyl-16-methoxy-N-(2-(2-methoxypyridin-4-yl)ethyl)- |
| | 10-methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 159 | (3R,9S,13S)-N-(2-(benzo[d][1,3]dioxol-5-yl)ethyl)-3-benzyl-9-isobutyl-17,18-dimethoxy-10-methyl- |
| | 5,8,11,15-tetraoxo-7-(((R)-tetrahydro-2H-pyran-2-yl)methyl)-2,3,4,5,6,7,8,9,10,11,12,13,14,15- |
| | tetradecahydrobenzo[p][1]oxa[4,7,10,14]tetraazacycloheptadecine-13-carboxamide |
| 160 | (3R,9S,13S)-N-(2-(benzo[d][1,3]dioxol-5-yl)ethyl)-3-benzyl-9-isobutyl-17,18-dimethoxy-10-methyl- |
| | 5,8,11,15-tetraoxo-7-(((S)-tetrahydro-2H-pyran-2-yl)methyl)-2,3,4,5,6,7,8,9,10,11,12,13,14,15- |
| | tetradecahydrobenzo[p][1]oxa[4,7,10,14]tetraazacycloheptadecine-13-carboxamide |
| 161 | (3R,9S,13S)-3-benzyl-9-isobutyl-17,18-dimethoxy-7,10-dimethyl-5,8,11,15-tetraoxo-N-(2-(2,3,4,5- |
| | tetrahydrobenzo[b]oxepin-8-yl)ethyl)-2,3,4,5,6,7,8,9,10,11,12,13,14,15- |
| | tetradecahydrobenzo[p][1]oxa[4,7,10,14]tetraazacycloheptadecine-13-carboxamide |
| 162 | (3RS,7S,13R)-N-(2-(benzo[d][1,3]dioxol-5-yl)ethyl)-13-benzyl-7-isobutyl-6-methyl-1,5,8,11-tetraoxo-9- |
| | (((R)-tetrahydro-2H-pyran-2-yl)methyl)-1,2,3,4,5,6,7,8,9,10,11,12,13,14-tetradecahydronaphtho[1,2- |
| | p][1]oxa[4,7,10,14]tetraazacycloheptadecine-3-carboxamide |
| 163 | (3RS,7S,13R)-N-(2-(benzo[d][1,3]dioxol-5-yl)ethyl)-13-benzyl-7-isobutyl-6-methyl-1,5,8,11-tetraoxo-9- |
| | (((S)-tetrahydro-2H-pyran-2-yl)methyl)-1,2,3,4,5,6,7,8,9,10,11,12,13,14-tetradecahydronaphtho[1,2- |
| | p][1]oxa[4,7,10,14]tetraazacycloheptadecine-3-carboxamide |
| 164 | (3RS,7S,13R)-13-benzyl-7-isobutyl-N-(3-methoxyphenethyl)-6-methyl-1,5,8,11-tetraoxo-9-(((R)- |
| | tetrahydro-2H-pyran-2-yl)methyl)-1,2,3,4,5,6,7,8,9,10,11,12,13,14-tetradecahydronaphtho[1,2- |
| | p][1]oxa[4,7,10,14]tetraazacycloheptadecine-3-carboxamide |
| 165 | (3RS,7S,13R)-13-benzyl-7-isobutyl-N-(3-methoxyphenethyl)-6-methyl-1,5,8,11-tetraoxo-9-(((S)- |
| | tetrahydro-2H-pyran-2-yl)methyl)-1,2,3,4,5,6,7,8,9,10,11,12,13,14-tetradecahydronaphtho[1,2- |
| | p][1]oxa[4,7,10,14]tetraazacycloheptadecine-3-carboxamide |
| 166 | (3′S,7′S,13′R)-13′-benzyl-N-((1-(3,4-dimethoxyphenyl)cyclopropyl)methyl)-20′-fluoro-7′-isobutyl-6′,9′- |
| | dimethyl-1′,5′,8′,11′-tetraoxo-2′,3′,4′,5′,6′,7′,8′,9′,11′,12′,13′,14′-dodecahydro-1′H-spiro[cyclopropane- |
| | 1,10′-[1]oxa[4,7,10,14]tetraazacycloheptadecino[17,16-c]quinoline]-3′-carboxamide |
| 167 | (3′S,7′S,13′R)-13′-benzyl-20′-fluoro-7′-isobutyl-6′,9′-dimethyl-N-(3-(methylsulfonyl)phenethyl)-1′,5′,8′,11′- |
| | tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 168 | (3′S,7′S,13′R)-13′-benzyl-N-(3-cyanophenethyl)-20′-fluoro-7′-isobutyl-6′,9′-dimethyl-1′,5′,8′,11′-tetraoxo- |
| | 2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 169 | (3′S,7′S,13′R)-13′-benzyl-20′-fluoro-7′-isobutyl-6′,9′-dimethyl-1′,5′,8′,11′-tetraoxo-N-(3- |
| | (trifluoromethyl)phenethyl)-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 170 | (3′S,7′S,13′R)-13′-benzyl-20′-fluoro-7′-isobutyl-6′,9′-dimethyl-N-(4-morpholinophenethyl)-1′,5′,8′,11′- |
| | tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 171 | (9S,13S,19aR,22R)-22-benzyl-5-fluoro-13-isobutyl-N-(2-(5-isopropyl-1,2,4-oxadiazol-3-yl)ethyl)-12- |
| | methyl-7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 172 | (3′S,7′S,13′R)-13′-benzyl-N-(2-(4-cyclopropyl-1H-pyrazol-1-yl)ethyl)-20′-fluoro-7′-isobutyl-6′,9′-dimethyl- |
| | 1′,5′,8′,11′-tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 173 | (3′S,7′S,13′R)-13′-benzyl-N-(2-(4-bromo-1H-pyrazol-1-yl)ethyl)-20′-fluoro-7′-isobutyl-6′,9′-dimethyl- |
| | 1′,5′,8′,11′-tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 174 | (3′S,7′S,13′R)-13′-benzyl-20′-fluoro-7′-isobutyl-N-(2-(4-(3-methoxypropyl)-1H-pyrazol-1-yl)ethyl)-6′,9′- |
| | dimethyl-1′,5′,8′,11′-tetraoxo-2′,3′,4′,5′,6′,7′,8′,9′,11′,12′,13′,14′-dodecahydro-1′H-spiro[cyclopropane- |
| | 1,10′-[1]oxa[4,7,10,14]tetraazacycloheptadecino[17,16-c]quinoline]-3′-carboxamide |
| 175 | (3′S,7′S,13′R)-13′-benzyl-N-(2,2-difluoro-2-(3-methoxyphenyl)ethyl)-7′-isobutyl-6′,9′-dimethyl-1′,5′,8′,11′- |
| | tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 176 | (7S,11S,17aR,20R)-20-benzyl-N-(2-(1-cyclopropyl-1H-pyrazol-4-yl)ethyl)-11-isobutyl-4-methoxy-10- |
| | methyl-5,9,12,18-tetraoxo-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 177 | (7S,11S,17aR,20R)-20-benzyl-11-isobutyl-4-methoxy-10-methyl-5,9,12,18-tetraoxo-N-(2-(3- |
| | (trifluoromethyl)isoxazol-5-yl)ethyl)-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 178 | (7S,11S,17aR,20R)-20-benzyl-11-isobutyl-4-methoxy-10-methyl-5,9,12,18-tetraoxo-N-(2-(3- |
| | (trifluoromethyl)-1,2,4-oxadiazol-5-yl)ethyl)-5,6,7,8,9,10,11,12,15,16,17,17a,18,19,20,21- |
| | hexadecahydro-14H-dipyrido[2,1-f:3′,4′-p][1]oxa[4,7,10,14]tetraazacycloheptadecine-7-carboxamide |
| 179 | (7S,11S,17aR,20R)-20-benzyl-N-(2-(4-cyclopropyl-1H-pyrazol-1-yl)ethyl)-11-isobutyl-4-methoxy-10- |
| | methyl-5,9,12,18-tetraoxo-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 180 | (7S,11S,17aR,20R)-20-benzyl-11-isobutyl-4-methoxy-N-(3-methoxyphenethyl)-10-methyl-5,9,12,18- |
| | tetraoxo-5,6,7,8,9,10,11,12,15,16,17,17a,18,19,20,21-hexadecahydro-14H-dipyrido[2,1-f:3′,4′- |
| | p][1]oxa[4,7,10,14]tetraazacycloheptadecine-7-carboxamide |
| 181 | (7S,11S,17aR,20R)-20-benzyl-11-isobutyl-4-methoxy-10-methyl-5,9,12,18-tetraoxo-N-(4-(pyrimidin-2- |
| | yl)phenethyl)-5,6,7,8,9,10,11,12,15,16,17,17a,18,19,20,21-hexadecahydro-14H-dipyrido[2,1-f:3′,4′- |
| | p][1]oxa[4,7,10,14]tetraazacycloheptadecine-7-carboxamide |
| 182 | (7S,11S,17aR,20R)-20-benzyl-11-isobutyl-4-methoxy-10-methyl-5,9,12,18-tetraoxo-N-(2-(3-phenyl- |
| | 1,2,4-oxadiazol-5-yl)ethyl)-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 183 | (7S,11S,17aR,20R)-20-benzyl-11-isobutyl-4-methoxy-10-methyl-5,9,12,18-tetraoxo-N-phenethyl- |
| | 5,6,7,8,9,10,11,12,15,16,17,17a,18,19,20,21-hexadecahydro-14H-dipyrido[2,1-f:3′,4′- |
| | p][1]oxa[4,7,10,14]tetraazacycloheptadecine-7-carboxamide |
| 184 | (7S,11S,17aR,20R)-20-benzyl-N-(4-bromophenethyl)-11-isobutyl-4-methoxy-10-methyl-5,9,12,18- |
| | tetraoxo-5,6,7,8,9,10,11,12,15,16,17,17a,18,19,20,21-hexadecahydro-14H-dipyrido[2,1-f:3′,4′- |
| | p][1]oxa[4,7,10,14]tetraazacycloheptadecine-7-carboxamide |
| 185 | (7S,11S,17aR,20R)-20-benzyl-11-isobutyl-4-methoxy-N-(4-methoxyphenethyl)-10-methyl-5,9,12,18- |
| | tetraoxo-5,6,7,8,9,10,11,12,15,16,17,17a,18,19,20,21-hexadecahydro-14H-dipyrido[2,1-f:3′,4′- |
| | p][1]oxa[4,7,10,14]tetraazacycloheptadecine-7-carboxamide |
| 186 | (7S,11S,17aR,20R)-20-benzyl-11-isobutyl-4-methoxy-10-methyl-N-(3-methylphenethyl)-5,9,12,18- |
| | tetraoxo-5,6,7,8,9,10,11,12,15,16,17,17a,18,19,20,21-hexadecahydro-14H-dipyrido[2,1-f:3′,4′- |
| | p][1]oxa[4,7,10,14]tetraazacycloheptadecine-7-carboxamide |
| 187 | (7S,11S,17aR,20R)-20-benzyl-N-(2-(2-cyclopropyl-2H-1,2,3-triazol-4-yl)ethyl)-11-isobutyl-4-methoxy- |
| | 10-methyl-5,9,12,18-tetraoxo-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 188 | (3′S,7′S,13′R)-13′-benzyl-20′-fluoro-7′-isobutyl-N-(2-(4-methoxy-1H-pyrazol-1-yl)ethyl)-6′,9′-dimethyl- |
| | 1′,5′,8′,11′-tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 189 | (7S,11S,17aR,20R)-20-benzyl-N-(3-ethynylphenethyl)-11-isobutyl-4-methoxy-10-methyl-5,9,12,18- |
| | tetraoxo-5,6,7,8,9,10,11,12,15,16,17,17a,18,19,20,21-hexadecahydro-14H-dipyrido[2,1-f:3′,4′- |
| | p][1]oxa[4,7,10,14]tetraazacycloheptadecine-7-carboxamide |
| 190 | (7S,11S,17aR,20R)-20-benzyl-N-(2-(5,6-dihydro-4H-cyclopenta[d]thiazol-2-yl)ethyl)-11-isobutyl-4- |
| | methoxy-10-methyl-5,9,12,18-tetraoxo-5,6,7,8,9,10,11,12,15,16,17,17a,18,19,20,21-hexadecahydro- |
| | 14H-dipyrido[2,1-f:3′,4′-p][1]oxa[4,7,10,14]tetraazacycloheptadecine-7-carboxamide |
| 191 | (7S,11S,17aR,20R)-20-benzyl-N-(2-(3-(difluoromethyl)isoxazol-5-yl)ethyl)-11-isobutyl-4-methoxy-10- |
| | methyl-5,9,12,18-tetraoxo-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 192 | (3R,9S,13S)-3-benzyl-9-isobutyl-17,18-dimethoxy-7,10-dimethyl-N-(4-methylphenethyl)-5,8,11,15- |
| | tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 193 | (3R,9S,13S)-3-benzyl-9-isobutyl-17,18-dimethoxy-N-(4-methoxyphenethyl)-7,10-dimethyl-5,8,11,15- |
| | tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 194 | (3R,9S,13S)-3-benzyl-N-(3-ethoxyphenethyl)-9-isobutyl-17,18-dimethoxy-7,10-dimethyl-5,8,11,15- |
| | tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 195 | (3R,9S,13S)-3-benzyl-9-isobutyl-17,18-dimethoxy-N-(3-methoxyphenethyl)-7,10-dimethyl-5,8,11,15- |
| | tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 196 | (3R,9S,13S)-3-benzyl-N-(2-fluorophenethyl)-9-isobutyl-17,18-dimethoxy-7,10-dimethyl-5,8,11,15- |
| | tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 197 | (3R,9S,13S)-3-benzyl-N-(4-(dimethylamino)phenethyl)-9-isobutyl-17,18-dimethoxy-7,10-dimethyl- |
| | 5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 198 | (3R,9S,13S)-N-(2-(benzo[d][1,3]dioxol-5-yl)ethyl)-3-benzyl-7-(2-cyclohexylethyl)-9-isobutyl-16-methoxy- |
| | 10-methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 199 | (3R,9S,13S)-3-benzyl-7-(2-cyclohexylethyl)-9-isobutyl-16-methoxy-N-(3-methoxyphenethyl)-10-methyl- |
| | 5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 200 | (3R,9S,13S)-3-benzyl-7-(2-cyclohexylethyl)-9-isobutyl-16-methoxy-N-(3-(methoxy-d3)phenethyl)-10- |
| | methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 201 | (3R,9S,13S)-3-benzyl-7-(2-cyclohexylethyl)-N-(3-ethoxyphenethyl)-9-isobutyl-16-methoxy-10-methyl- |
| | 5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 202 | (3R,9S,13S)-3-benzyl-7-(2-cyclohexylethyl)-9-isobutyl-16-methoxy-10-methyl-N-(4-methylphenethyl)- |
| | 5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 203 | (3R,9S,13S)-3-benzyl-7-(2-cyclohexylethyl)-N-(4-(dimethylamino)phenethyl)-9-isobutyl-16-methoxy-10- |
| | methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 204 | (3R,9S,13S)-3-benzyl-7-(2-cyclohexylethyl)-N-(2-fluorophenethyl)-9-isobutyl-16-methoxy-10-methyl- |
| | 5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 205 | (3R,9S,13S)-3-benzyl-7-(2-cyclohexylethyl)-N-(3,5-dimethoxyphenethyl)-9-isobutyl-16-methoxy-10- |
| | methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 206 | (3R,9S,13S)-3-benzyl-7-(2-cyclohexylethyl)-9-isobutyl-16-methoxy-10-methyl-5,8,11,15-tetraoxo-N-(2- |
| | (thiophen-2-yl)ethyl)-2,3,4,5,6,7,8,9,10,11,12,13,14,15- |
| | tetradecahydrobenzo[p][1]oxa[4,7,10,14]tetraazacycloheptadecine-13-carboxamide |
| 207 | (3R,9S,13S)-3-benzyl-7-(2-cyclohexylethyl)-9-isobutyl-16-methoxy-N-(2-(5-methoxypyridin-2-yl)ethyl)- |
| | 10-methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 208 | (3R,9S,13S)-3-benzyl-7-(2-cyclohexylethyl)-N-(2-(furan-2-yl)ethyl)-9-isobutyl-16-methoxy-10-methyl- |
| | 5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 209 | (3R,9S,13S)-3-benzyl-7-(2-cyclohexylethyl)-N-(2-(3,5-dimethyl-1H-pyrazol-1-yl)ethyl)-9-isobutyl-16- |
| | methoxy-10-methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 210 | (3R,9S,13S)-3-benzyl-7-(2-cyclohexylethyl)-9-isobutyl-16-methoxy-N-(3-methoxypropyl)-10-methyl- |
| | 5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 211 | (3R,9S,13S)-3-benzyl-7-(2-cyclohexylethyl)-9-isobutyl-16-methoxy-10-methyl-5,8,11,15-tetraoxo-N- |
| | phenethyl-2,3,4,5,6,7,8,9,10,11,12,13,14,15- |
| | tetradecahydrobenzo[p][1]oxa[4,7,10,14]tetraazacycloheptadecine-13-carboxamide |
| 212 | (3R,9S,13S)-3-benzyl-7-(2-cyclohexylethyl)-9-isobutyl-16-methoxy-10-methyl-5,8,11,15-tetraoxo-N-(2- |
| | ((RS)-tetrahydro-2H-pyran-2-yl)ethyl)-2,3,4,5,6,7,8,9,10,11,12,13,14,15- |
| | tetradecahydrobenzo[p][1]oxa[4,7,10,14]tetraazacycloheptadecine-13-carboxamide |
| 213 | (3R,9S,13S)-3-benzyl-7-(2-cyclohexylethyl)-N-(2-((RS)-2,3-dihydrobenzo[b][1,4]dioxin-2-yl)ethyl)-9- |
| | isobutyl-16-methoxy-10-methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 214 | (3R,9S,13S)-3-benzyl-7-(2-cyclohexylethyl)-9-isobutyl-16-methoxy-10-methyl-N-(2-(3-methylisoxazol-5- |
| | yl)ethyl)-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 215 | (3R,9S,13S)-3-benzyl-7-(2-cyclohexylethyl)-N-(4-ethylphenethyl)-9-isobutyl-16-methoxy-10-methyl- |
| | 5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 216 | (3R,9S,13S)-3-benzyl-7-(2-cyclohexylethyl)-9-isobutyl-16-methoxy-N-(5-methoxypentyl)-10-methyl- |
| | 5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 217 | (3R,9S,13S)-N-(4-(1H-pyrazol-1-yl)phenethyl)-3-benzyl-7-(2-cyclohexylethyl)-9-isobutyl-16-methoxy-10- |
| | methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 218 | (3R,9S,13S)-N-(4-amino-3-methoxyphenethyl)-3-benzyl-7-(2-cyclohexylethyl)-9-isobutyl-16-methoxy- |
| | 10-methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 219 | (3R,9S,13RS)-3-benzyl-7-(2-cyclohexylethyl)-9-isobutyl-16-methoxy-N-(4-methoxyphenethyl)-10- |
| | methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 220 | (3R,9S,13S)-3-benzyl-7-(2-cyclohexylethyl)-9-isobutyl-16-methoxy-N-(2-(6-methoxypyridin-2-yl)ethyl)- |
| | 10-methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 221 | (9S,13S,19aR,22R)-22-benzyl-13-isobutyl-12-methyl-7,11,14,20-tetraoxo-N-(2-(3- |
| | (trifluoromethyl)isoxazol-5-yl)ethyl)-7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 222 | (4aR,7R,18S,22S)-7-benzyl-22-isobutyl-N-(3-methoxyphenethyl)-21-methyl-5,16,20,23-tetraoxo- |
| | 1,2,4,4a,5,6,7,8,16,17,18,19,20,21,22,23-hexadecahydronaphtho[1,2-p][1,4]oxazino[3,4- |
| | f][1]oxa[4,7,10,14]tetraazacycloheptadecine-18-carboxamide |
| 223 | (7R,13S,17S)-N-(2-(benzo[d][1,3]dioxol-5-yl)ethyl)-7-benzyl-13-isobutyl-11,14-dimethyl-9,12,15,19- |
| | tetraoxo-6,7,8,9,10,11,12,13,14,15,16,17,18,19-tetradecahydro-[1,3]dioxolo[4′,5′:4,5]benzo[1,2- |
| | p][1]oxa[4,7,10,14]tetraazacycloheptadecine-17-carboxamide |
| 224 | (7R,13S,17S)-7-benzyl-13-isobutyl-N-(3-methoxyphenethyl)-11,14-dimethyl-9,12,15,19-tetraoxo- |
| | 6,7,8,9,10,11,12,13,14,15,16,17,18,19-tetradecahydro-[1,3]dioxolo[4′,5′:4,5]benzo[1,2- |
| | p][1]oxa[4,7,10,14]tetraazacycloheptadecine-17-carboxamide |
| 225 | (7R,13S,17S)-7-benzyl-N-(3-ethoxyphenethyl)-13-isobutyl-11,14-dimethyl-9,12,15,19-tetraoxo- |
| | 6,7,8,9,10,11,12,13,14,15,16,17,18,19-tetradecahydro-[1,3]dioxolo[4′,5′:4,5]benzo[1,2- |
| | p][1]oxa[4,7,10,14]tetraazacycloheptadecine-17-carboxamide |
| 226 | (7R,13S,17S)-7-benzyl-N-(2-(2,3-dihydrobenzo[b][1,4]dioxin-6-yl)ethyl)-13-isobutyl-11,14-dimethyl- |
| | 9,12,15,19-tetraoxo-6,7,8,9,10,11,12,13,14,15,16,17,18,19-tetradecahydro- |
| | [1,3]dioxolo[4′,5′:4,5]benzo[1,2-p][1]oxa[4,7,10,14]tetraazacycloheptadecine-17-carboxamide |
| 227 | (7R,13S,17S)-7-benzyl-N-(4-ethylphenethyl)-13-isobutyl-11,14-dimethyl-9,12,15,19-tetraoxo- |
| | 6,7,8,9,10,11,12,13,14,15,16,17,18,19-tetradecahydro-[1,3]dioxolo[4′,5′:4,5]benzo[1,2- |
| | p][1]oxa[4,7,10,14]tetraazacycloheptadecine-17-carboxamide |
| 228 | (3R,9S,13S)-N-(2-(benzo[d][1,3]dioxol-5-yl)ethyl)-3-benzyl-9-isobutyl-17-methoxy-7,10-dimethyl- |
| | 5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 229 | (3R,9S,13S)-N-(2-(benzo[d][1,3]dioxol-5-yl)ethyl)-3-benzyl-9-isobutyl-7,10,17-trimethyl-5,8,11,15- |
| | tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 230 | (3R,9S,13S)-3-benzyl-9-isobutyl-N-(3-methoxyphenethyl)-7,10,17-trimethyl-5,8,11,15-tetraoxo- |
| | 2,3,4,5,6,7,8,9,10,11,12,13,14,15-tetradecahydrobenzo[p][1]oxa[4,7,10,14]tetraazacycloheptadecine- |
| | 13-carboxamide |
| 231 | (3R,9S,13S)-3-benzyl-9-isobutyl-17-methoxy-N-(3-methoxyphenethyl)-7,10-dimethyl-5,8,11,15- |
| | tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 232 | (3R,9S,13S)-3-benzyl-9-isobutyl-18-methoxy-N-(3-methoxyphenethyl)-7,10-dimethyl-5,8,11,15- |
| | tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 233 | (3R,9S,13S)-3-benzyl-9-isobutyl-N-(3-methoxyphenethyl)-7,10,18-trimethyl-5,8,11,15-tetraoxo- |
| | 2,3,4,5,6,7,8,9,10,11,12,13,14,15-tetradecahydrobenzo[p][1]oxa[4,7,10,14]tetraazacycloheptadecine- |
| | 13-carboxamide |
| 234 | (3R,9S,13S)-N-(2-(benzo[d][1,3]dioxol-5-yl)ethyl)-3-benzyl-17-fluoro-9-isobutyl-7,10-dimethyl-5,8,11,15- |
| | tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 235 | (3R,9S,13S)-N-(2-(benzo[d][1,3]dioxol-5-yl)ethyl)-3-benzyl-9-isobutyl-7,10,16-trimethyl-5,8,11,15- |
| | tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 236 | (3R,9S,13S)-N-(2-(benzo[d][1,3]dioxol-5-yl)ethyl)-3-benzyl-9-isobutyl-7,10-dimethyl-5,8,11,15-tetraoxo- |
| | 2,3,4,5,6,7,8,9,10,11,12,13,14,15-tetradecahydronaphtho[2,3- |
| | p][1]oxa[4,7,10,14]tetraazacycloheptadecine-13-carboxamide |
| 237 | (3R,9S,13S)-3-benzyl-17-chloro-9-isobutyl-N-(3-methoxyphenethyl)-7,10-dimethyl-5,8,11,15-tetraoxo- |
| | 2,3,4,5,6,7,8,9,10,11,12,13,14,15-tetradecahydrobenzo[p][1]oxa[4,7,10,14]tetraazacycloheptadecine- |
| | 13-carboxamide |
| 238 | (3R,9S,13S)-3-benzyl-9-isobutyl-N-(3-methoxyphenethyl)-7,10-dimethyl-5,8,11,15-tetraoxo- |
| | 2,3,4,5,6,7,8,9,10,11,12,13,14,15-tetradecahydronaphtho[2,3- |
| | p][1]oxa[4,7,10,14]tetraazacycloheptadecine-13-carboxamide |
| 239 | (3R,9S,13S)-3-benzyl-N-(4-ethylphenethyl)-9-isobutyl-16-methoxy-7,10-dimethyl-5,8,11,15-tetraoxo- |
| | 2,3,4,5,6,7,8,9,10,11,12,13,14,15-tetradecahydrobenzo[p][1]oxa[4,7,10,14]tetraazacycloheptadecine- |
| | 13-carboxamide |
| 240 | (6R,12S,16S)-6-benzyl-12-isobutyl-N-(3-methoxyphenethyl)-10,13-dimethyl-8,11,14,18-tetraoxo- |
| | 5,6,7,8,9,10,11,12,13,14,15,16,17,18-tetradecahydrothieno[2,3- |
| | p][1]oxa[4,7,10,14]tetraazacycloheptadecine-16-carboxamide |
| 241 | (6R,12S,16S)-6-benzyl-12-isobutyl-N-(3-methoxyphenethyl)-2,10,13-trimethyl-8,11,14,18-tetraoxo- |
| | 5,6,7,8,9,10,11,12,13,14,15,16,17,18-tetradecahydrothiazolo[5,4- |
| | p][1]oxa[4,7,10,14]tetraazacycloheptadecine-16-carboxamide |
| 242 | (3R,9S,13S)-N-(2-(benzo[d][1,3]dioxol-5-yl)ethyl)-3-benzyl-7-(2-cyclohexylethyl)-9-isobutyl-16- |
| | (methoxy-d3)-10-methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 243 | (3R,9S,13S)-3-benzyl-7-(2-cyclohexylethyl)-9-isobutyl-16-(methoxy-d3)-N-(3-methoxyphenethyl)-10- |
| | methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 244 | (3R,9S,13S)-3-benzyl-7-(2-cyclohexylethyl)-9-isobutyl-16-(methoxy-d3)-N-(3-(methoxy-d3)phenethyl)- |
| | 10-methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 245 | (3R,9S,13S)-3-benzyl-N-(2-(chroman-7-yl)ethyl)-9-isobutyl-17-methoxy-7,10-dimethyl-5,8,11,15- |
| | tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 246 | (3R,9S,13S)-N-(2-(benzo[d][1,3]dioxol-5-yl)ethyl)-3-benzyl-7-(2-cyclohexylethyl)-9-isobutyl-10,16- |
| | dimethyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 247 | (3R,9S,13S)-3-benzyl-7-(2-cyclohexylethyl)-9-isobutyl-N-(3-methoxyphenethyl)-10,16-dimethyl- |
| | 5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 248 | (3R,9S,13S)-N-(2-(benzo[d][1,3]dioxol-5-yl)ethyl)-3-benzyl-7-(2-cyclohexylethyl)-9-isobutyl-17-methoxy- |
| | 10-methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 249 | (3R,9S,13S)-3-benzyl-7-(2-cyclohexylethyl)-9-isobutyl-17-methoxy-N-(3-methoxyphenethyl)-10-methyl- |
| | 5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 250 | (3R,9S,13S)-N-(2-(benzo[d][1,3]dioxol-5-yl)ethyl)-3-benzyl-7-(2-cyclohexylethyl)-9-isobutyl-10-methyl- |
| | 5,8,11,15-tetraoxo-2,3,4,5,6,7,8,9,10,11,12,13,14,15-tetradecahydronaphtho[2,3- |
| | p][1]oxa[4,7,10,14]tetraazacycloheptadecine-13-carboxamide |
| 251 | (3R,9S,13S)-3-benzyl-7-(2-cyclohexylethyl)-9-isobutyl-N-(3-methoxyphenethyl)-10-methyl-5,8,11,15- |
| | tetraoxo-2,3,4,5,6,7,8,9,10,11,12,13,14,15-tetradecahydronaphtho[2,3- |
| | p][1]oxa[4,7,10,14]tetraazacycloheptadecine-13-carboxamide |
| 252 | (3R,9S,13S)-3-benzyl-N-(4-ethylphenethyl)-9-isobutyl-7,10-dimethyl-5,8,11,15-tetraoxo- |
| | 2,3,4,5,6,7,8,9,10,11,12,13,14,15-tetradecahydropyrido[3,4- |
| | p][1]oxa[4,7,10,14]tetraazacycloheptadecine-13-carboxamide |
| 253 | (3R,9S,13S)-N-(2-(benzo[d][1,3]dioxol-5-yl)ethyl)-3-benzyl-17-chloro-7-(2-cyclohexylethyl)-9-isobutyl- |
| | 10-methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 254 | (3R,9S,13S)-3-benzyl-17-chloro-7-(2-cyclohexylethyl)-9-isobutyl-N-(3-methoxyphenethyl)-10-methyl- |
| | 5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 255 | (3R,9S,13S)-N-(2-(benzo[d][1,3]dioxol-5-yl)ethyl)-3-benzyl-7-(2-cyclohexylethyl)-9-isobutyl-16,18- |
| | dimethoxy-10-methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 256 | (3R,9S,13S)-3-benzyl-7-(2-cyclohexylethyl)-9-isobutyl-16,18-dimethoxy-N-(3-methoxyphenethyl)-10- |
| | methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 257 | (3R,13S)-N-(2-(benzo[d][1,3]dioxol-5-yl)ethyl)-3-benzyl-7-(2-cyclohexylethyl)-16-methoxy-10-methyl- |
| | 5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 258 | (3R,13S)-3-benzyl-7-(2-cyclohexylethyl)-16-methoxy-N-(3-methoxyphenethyl)-10-methyl-5,8,11,15- |
| | tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 259 | (7S,11S,20R,22aR)-20-benzyl-N-(4-bromophenethyl)-7-isobutyl-14-methoxy-8-methyl-6,9,13,22- |
| | tetraoxo-1,3,4,6,7,8,9,10,11,12,13,19,20,21,22,22a-hexadecahydrobenzo[p][1,4]oxazino[3,4- |
| | f][1]oxa[4,7,10,14]tetraazacycloheptadecine-11-carboxamide |
| 260 | (7S,11S,20R,22aR)-20-benzyl-7-isobutyl-14-methoxy-8-methyl-6,9,13,22-tetraoxo-N-(2-(2,3,4,5- |
| | tetrahydrobenzo[b]oxepin-8-yl)ethyl)-1,3,4,6,7,8,9,10,11,12,13,19,20,21,22,22a- |
| | hexadecahydrobenzo[p][1,4]oxazino[3,4-f][1]oxa[4,7,10,14]tetraazacycloheptadecine-11-carboxamide |
| 261 | (3R,9S,13S)-3-benzyl-N-(2-(chroman-7-yl)ethyl)-7-(2-cyclohexylethyl)-9-isobutyl-10-methyl-5,8,11,15- |
| | tetraoxo-2,3,4,5,6,7,8,9,10,11,12,13,14,15-tetradecahydropyrido[3,4- |
| | p][1]oxa[4,7,10,14]tetraazacycloheptadecine-13-carboxamide |
| 262 | (3R,9S,13S)-3-benzyl-7-(2-cyclohexylethyl)-N-(2-(2,3-dihydrobenzofuran-6-yl)ethyl)-9-isobutyl-10- |
| | methyl-5,8,11,15-tetraoxo-2,3,4,5,6,7,8,9,10,11,12,13,14,15-tetradecahydropyrido[3,4- |
| | p][1]oxa[4,7,10,14]tetraazacycloheptadecine-13-carboxamide |
| 263 | (3R,9S,13S)-N-(2-(benzo[d][1,3]dioxol-5-yl)ethyl)-3-benzyl-7-(2-cyclohexylethyl)-16-methoxy-9,10- |
| | dimethyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 264 | (3R,9S,13S)-3-benzyl-7-(2-cyclohexylethyl)-16-methoxy-N-(3-methoxyphenethyl)-9,10-dimethyl- |
| | 5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 265 | (3R,9S,13S)-N-(2-(benzo[d][1,3]dioxol-5-yl)ethyl)-3-benzyl-7-(2-cyclohexylethyl)-9-isobutyl-10-methyl- |
| | 5,8,11,15-tetraoxo-2,3,4,5,6,7,8,9,10,11,12,13,14,15-tetradecahydropyrido[4,3- |
| | p][1]oxa[4,7,10,14]tetraazacycloheptadecine-13-carboxamide |
| 266 | (3R,9S,13S)-3-benzyl-7-(2-cyclohexylethyl)-9-isobutyl-N-(3-methoxyphenethyl)-10-methyl-5,8,11,15- |
| | tetraoxo-2,3,4,5,6,7,8,9,10,11,12,13,14,15-tetradecahydropyrido[4,3- |
| | p][1]oxa[4,7,10,14]tetraazacycloheptadecine-13-carboxamide |
| 267 | (3S,7S,13R)-N-(2-(benzo[d][1,3]dioxol-5-yl)ethyl)-13-benzyl-9-(2-cyclohexylethyl)-7-isobutyl-6-methyl- |
| | 1,5,8,11-tetraoxo-1,2,3,4,5,6,7,8,9,10,11,12,13,14-tetradecahydronaphtho[1,2- |
| | p][1]oxa[4,7,10,14]tetraazacycloheptadecine-3-carboxamide |
| 268 | (3S,7S,13R)-13-benzyl-9-(2-cyclohexylethyl)-7-isobutyl-N-(3-methoxyphenethyl)-6-methyl-1,5,8,11- |
| | tetraoxo-1,2,3,4,5,6,7,8,9,10,11,12,13,14-tetradecahydronaphtho[1,2- |
| | p][1]oxa[4,7,10,14]tetraazacycloheptadecine-3-carboxamide |
| 269 | (3S,7S,13R)-N-(2-(benzo[d][1,3]dioxol-5-yl)ethyl)-13-benzyl-7-isobutyl-6,9-dimethyl-1,5,8,11-tetraoxo- |
| | 1,2,3,4,5,6,7,8,9,10,11,12,13,14-tetradecahydronaphtho[1,2- |
| | p][1]oxa[4,7,10,14]tetraazacycloheptadecine-3-carboxamide |
| 270 | (3R,9S,13S)-N-(2-(benzo[d][1,3]dioxol-5-yl)ethyl)-3-benzyl-7-(2-cyclohexylethyl)-9-isobutyl-10,18- |
| | dimethyl-5,8,11,15-tetraoxo-2,3,4,5,6,7,8,9,10,11,12,13,14,15-tetradecahydropyrido[3,4- |
| | p][1]oxa[4,7,10,14]tetraazacycloheptadecine-13-carboxamide |
| 271 | (3R,9S,13S)-3-benzyl-7-(2-cyclohexylethyl)-9-isobutyl-N-(3-methoxyphenethyl)-10,18-dimethyl- |
| | 5,8,11,15-tetraoxo-2,3,4,5,6,7,8,9,10,11,12,13,14,15-tetradecahydropyrido[3,4- |
| | p][1]oxa[4,7,10,14]tetraazacycloheptadecine-13-carboxamide |
| 272 | (3R,9S,13S)-N-(2-(benzo[d][1,3]dioxol-5-yl)ethyl)-3-benzyl-7-(2-cyclohexylethyl)-16,18-dimethoxy-9,10- |
| | dimethyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 273 | (3R,9S,13S)-3-benzyl-7-(2-cyclohexylethyl)-16,18-dimethoxy-N-(3-methoxyphenethyl)-9,10-dimethyl- |
| | 5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 274 | (3R,9S,13RS)-N-(2-(benzo[d][1,3]dioxol-5-yl)ethyl)-3-benzyl-7-(2-cyclohexylethyl)-9- |
| | (cyclopropylmethyl)-16-methoxy-10-methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 275 | (3R,9S,13S)-3-benzyl-N-(2-(chroman-7-yl)ethyl)-7-(2-cyclohexylethyl)-9-isobutyl-16,18-dimethoxy-10- |
| | methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 276 | (3R,9S,13S)-3-benzyl-7-(2-cyclohexylethyl)-N-(2-(2,3-dihydrobenzofuran-6-yl)ethyl)-9-isobutyl-16,18- |
| | dimethoxy-10-methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 277 | (3R,9S,13S)-3-benzyl-16-fluoro-9-isobutyl-N-(3-methoxyphenethyl)-7,10-dimethyl-5,8,11,15-tetraoxo- |
| | 2,3,4,5,6,7,8,9,10,11,12,13,14,15-tetradecahydrobenzo[p][1]oxa[4,7,10,14]tetraazacycloheptadecine- |
| | 13-carboxamide |
| 278 | (3R,9S,13S)-N-(2-(benzo[d][1,3]dioxol-5-yl)ethyl)-3-benzyl-16-fluoro-9-isobutyl-7,10-dimethyl-5,8,11,15- |
| | tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 279 | (3R,9S,13S)-N-(2-(benzo[d][1,3]dioxol-5-yl)ethyl)-3-benzyl-16-chloro-9-isobutyl-7,10-dimethyl- |
| | 5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 280 | (7S,11S,20R,22aR)-20-benzyl-14-ethoxy-7-isobutyl-N-(3-methoxyphenethyl)-8-methyl-6,9,13,22- |
| | tetraoxo-1,3,4,6,7,8,9,10,11,12,13,19,20,21,22,22a-hexadecahydro-2H-benzo[p]pyrido[2,1- |
| | f][1]oxa[4,7,10,14]tetraazacycloheptadecine-11-carboxamide |
| 281 | (3R,9S,13S)-N-(2-(benzo[d][1,3]dioxol-5-yl)ethyl)-3-benzyl-7-(2-cyclohexylethyl)-9-isobutyl-16-methoxy- |
| | 10-methyl-5,8,11,15-tetraoxo-2,3,4,5,6,7,8,9,10,11,12,13,14,15-tetradecahydropyrido[3,4- |
| | p][1]oxa[4,7,10,14]tetraazacycloheptadecine-13-carboxamide |
| 282 | (3R,9S,13S)-3-benzyl-7-(2-cyclohexylethyl)-9-isobutyl-16-methoxy-N-(3-methoxyphenethyl)-10-methyl- |
| | 5,8,11,15-tetraoxo-2,3,4,5,6,7,8,9,10,11,12,13,14,15-tetradecahydropyrido[3,4- |
| | p][1]oxa[4,7,10,14]tetraazacycloheptadecine-13-carboxamide |
| 283 | (7S,11S,20R,22aR)-20-benzyl-N-(2-(2,3-dihydrobenzo[b][1,4]dioxin-6-yl)ethyl)-14-ethoxy-7-isobutyl-8- |
| | methyl-6,9,13,22-tetraoxo-1,3,4,6,7,8,9,10,11,12,13,19,20,21,22,22a-hexadecahydro-2H- |
| | benzo[p]pyrido[2,1-f][1]oxa[4,7,10,14]tetraazacycloheptadecine-11-carboxamide |
| 284 | (3RS,7S,13R)-N-(2-(benzo[d][1,3]dioxol-5-yl)ethyl)-13-benzyl-7-(cyclopentylmethyl)-6-methyl-1,5,8,11- |
| | tetraoxo-9-(((S)-tetrahydro-2H-pyran-2-yl)methyl)-1,2,3,4,5,6,7,8,9,10,11,12,13,14- |
| | tetradecahydronaphtho[1,2-p][1]oxa[4,7,10,14]tetraazacycloheptadecine-3-carboxamide |
| 285 | (3RS,7S,13R)-N-(2-(benzo[d][1,3]dioxol-5-yl)ethyl)-13-benzyl-7-(cyclohexylmethyl)-6-methyl-1,5,8,11- |
| | tetraoxo-9-(((S)-tetrahydro-2H-pyran-2-yl)methyl)-1,2,3,4,5,6,7,8,9,10,11,12,13,14- |
| | tetradecahydronaphtho[1,2-p][1]oxa[4,7,10,14]tetraazacycloheptadecine-3-carboxamide |
| 286 | (3RS,7S,13R)-N-(2-(benzo[d][1,3]dioxol-5-yl)ethyl)-13-benzyl-7-(cyclopentylmethyl)-6-methyl-1,5,8,11- |
| | tetraoxo-9-(((R)-tetrahydro-2H-pyran-2-yl)methyl)-1,2,3,4,5,6,7,8,9,10,11,12,13,14- |
| | tetradecahydronaphtho[1,2-p][1]oxa[4,7,10,14]tetraazacycloheptadecine-3-carboxamide |
| 287 | (3RS,7S,13R)-N-(2-(benzo[d][1,3]dioxol-5-yl)ethyl)-13-benzyl-7-(cyclohexylmethyl)-6-methyl-1,5,8,11- |
| | tetraoxo-9-(((R)-tetrahydro-2H-pyran-2-yl)methyl)-1,2,3,4,5,6,7,8,9,10,11,12,13,14- |
| | tetradecahydronaphtho[1,2-p][1]oxa[4,7,10,14]tetraazacycloheptadecine-3-carboxamide |
| 288 | (3RS,7S,13R)-N-(2-(benzo[d][1,3]dioxol-5-yl)ethyl)-13-benzyl-7-(cyclopentylmethyl)-6-methyl-1,5,8,11- |
| | tetraoxo-9-(2-((SR)-tetrahydro-2H-pyran-2-yl)ethyl)-1,2,3,4,5,6,7,8,9,10,11,12,13,14- |
| | tetradecahydronaphtho[1,2-p][1]oxa[4,7,10,14]tetraazacycloheptadecine-3-carboxamide |
| 289 | (3RS,7S,13R)-N-(2-(benzo[d][1,3]dioxol-5-yl)ethyl)-13-benzyl-7-(cyclohexylmethyl)-6-methyl-1,5,8,11- |
| | tetraoxo-9-(2-((SR)-tetrahydro-2H-pyran-2-yl)ethyl)-1,2,3,4,5,6,7,8,9,10,11,12,13,14- |
| | tetradecahydronaphtho[1,2-p][1]oxa[4,7,10,14]tetraazacycloheptadecine-3-carboxamide |
| 290 | (3RS,7S,13R)-N-(2-(benzo[d][1,3]dioxol-5-yl)ethyl)-13-benzyl-9-(2-cyclohexylethyl)-7- |
| | (cyclopentylmethyl)-6-methyl-1,5,8,11-tetraoxo-1,2,3,4,5,6,7,8,9,10,11,12,13,14- |
| | tetradecahydronaphtho[1,2-p][1]oxa[4,7,10,14]tetraazacycloheptadecine-3-carboxamide |
| 291 | (3RS,7S,13R)-N-(2-(benzo[d][1,3]dioxol-5-yl)ethyl)-13-benzyl-9-(2-cyclohexylethyl)-7- |
| | (cyclohexylmethyl)-6-methyl-1,5,8,11-tetraoxo-1,2,3,4,5,6,7,8,9,10,11,12,13,14- |
| | tetradecahydronaphtho[1,2-p][1]oxa[4,7,10,14]tetraazacycloheptadecine-3-carboxamide |
| 292 | (3RS,7S,13R)-N-(2-(benzo[d][1,3]dioxol-5-yl)ethyl)-13-benzyl-7-(cyclobutylmethyl)-9-(2- |
| | cyclohexylethyl)-6-methyl-1,5,8,11-tetraoxo-1,2,3,4,5,6,7,8,9,10,11,12,13,14- |
| | tetradecahydronaphtho[1,2-p][1]oxa[4,7,10,14]tetraazacycloheptadecine-3-carboxamide |
| 293 | (3RS,7S,13R)-13-benzyl-7-(cyclopentylmethyl)-N-(3-methoxyphenethyl)-6-methyl-1,5,8,11-tetraoxo-9- |
| | (((S)-tetrahydro-2H-pyran-2-yl)methyl)-1,2,3,4,5,6,7,8,9,10,11,12,13,14-tetradecahydronaphtho[1,2- |
| | p][1]oxa[4,7,10,14]tetraazacycloheptadecine-3-carboxamide |
| 294 | (3RS,7S,13R)-13-benzyl-7-(cyclohexylmethyl)-N-(3-methoxyphenethyl)-6-methyl-1,5,8,11-tetraoxo-9- |
| | (((S)-tetrahydro-2H-pyran-2-yl)methyl)-1,2,3,4,5,6,7,8,9,10,11,12,13,14-tetradecahydronaphtho[1,2- |
| | p][1]oxa[4,7,10,14]tetraazacycloheptadecine-3-carboxamide |
| 295 | (3RS,7S,13R)-13-benzyl-7-(cyclopentylmethyl)-N-(3-methoxyphenethyl)-6-methyl-1,5,8,11-tetraoxo-9- |
| | (((R)-tetrahydro-2H-pyran-2-yl)methyl)-1,2,3,4,5,6,7,8,9,10,11,12,13,14-tetradecahydronaphtho[1,2- |
| | p][1]oxa[4,7,10,14]tetraazacycloheptadecine-3-carboxamide |
| 296 | (3RS,7S,13R)-13-benzyl-7-(cyclohexylmethyl)-N-(3-methoxyphenethyl)-6-methyl-1,5,8,11-tetraoxo-9- |
| | (((R)-tetrahydro-2H-pyran-2-yl)methyl)-1,2,3,4,5,6,7,8,9,10,11,12,13,14-tetradecahydronaphtho[1,2- |
| | p][1]oxa[4,7,10,14]tetraazacycloheptadecine-3-carboxamide |
| 297 | (3RS,7S,13R)-13-benzyl-7-isobutyl-N-(3-methoxyphenethyl)-6-methyl-1,5,8,11-tetraoxo-9-(2-((SR)- |
| | tetrahydro-2H-pyran-2-yl)ethyl)-1,2,3,4,5,6,7,8,9,10,11,12,13,14-tetradecahydronaphtho[1,2- |
| | p][1]oxa[4,7,10,14]tetraazacycloheptadecine-3-carboxamide |
| 298 | (3RS,7S,13R)-13-benzyl-7-(cyclopentylmethyl)-N-(3-methoxyphenethyl)-6-methyl-1,5,8,11-tetraoxo-9- |
| | (2-((SR)-tetrahydro-2H-pyran-2-yl)ethyl)-1,2,3,4,5,6,7,8,9,10,11,12,13,14-tetradecahydronaphtho[1,2- |
| | p][1]oxa[4,7,10,14]tetraazacycloheptadecine-3-carboxamide |
| 299 | (3RS,7S,13R)-13-benzyl-7-(cyclohexylmethyl)-N-(3-methoxyphenethyl)-6-methyl-1,5,8,11-tetraoxo-9-(2- |
| | ((SR)-tetrahydro-2H-pyran-2-yl)ethyl)-1,2,3,4,5,6,7,8,9,10,11,12,13,14-tetradecahydronaphtho[1,2- |
| | p][1]oxa[4,7,10,14]tetraazacycloheptadecine-3-carboxamide |
| 300 | (3RS,7S,13R)-13-benzyl-9-(2-cyclohexylethyl)-7-(cyclopentylmethyl)-N-(3-methoxyphenethyl)-6-methyl- |
| | 1,5,8,11-tetraoxo-1,2,3,4,5,6,7,8,9,10,11,12,13,14-tetradecahydronaphtho[1,2- |
| | p][1]oxa[4,7,10,14]tetraazacycloheptadecine-3-carboxamide |
| 301 | (3RS,7S,13R)-13-benzyl-9-(2-cyclohexylethyl)-7-(cyclohexylmethyl)-N-(3-methoxyphenethyl)-6-methyl- |
| | 1,5,8,11-tetraoxo-1,2,3,4,5,6,7,8,9,10,11,12,13,14-tetradecahydronaphtho[1,2- |
| | p][1]oxa[4,7,10,14]tetraazacycloheptadecine-3-carboxamide |
| 302 | (3RS,7S,13R)-13-benzyl-7-(cyclobutylmethyl)-9-(2-cyclohexylethyl)-N-(3-methoxyphenethyl)-6-methyl- |
| | 1,5,8,11-tetraoxo-1,2,3,4,5,6,7,8,9,10,11,12,13,14-tetradecahydronaphtho[1,2- |
| | p][1]oxa[4,7,10,14]tetraazacycloheptadecine-3-carboxamide |
| 303 | (3RS,7S,13R)-13-benzyl-9-(2-cyclohexylethyl)-7-isobutyl-N-(3-methoxyphenethyl)-6-methyl-1,5,8,11- |
| | tetraoxo-1,2,3,4,5,6,7,8,9,10,11,12,13,14-tetradecahydronaphtho[1,2- |
| | p][1]oxa[4,7,10,14]tetraazacycloheptadecine-3-carboxamide |
| 304 | (3R,9S,13S)-N-(2-(benzo[d][1,3]dioxol-5-yl)ethyl)-3-benzyl-9-isobutyl-16-methoxy-10-methyl-5,8,11,15- |
| | tetraoxo-7-(2-((RS)-tetrahydro-2H-pyran-2-yl)ethyl)-2,3,4,5,6,7,8,9,10,11,12,13,14,15- |
| | tetradecahydrobenzo[p][1]oxa[4,7,10,14]tetraazacycloheptadecine-13-carboxamide |
| 305 | (3R,9S,13S)-N-(2-(benzo[d][1,3]dioxol-5-yl)ethyl)-3-benzyl-9-isobutyl-16-methoxy-10-methyl-5,8,11,15- |
| | tetraoxo-7-(((R)-tetrahydro-2H-pyran-2-yl)methyl)-2,3,4,5,6,7,8,9,10,11,12,13,14,15- |
| | tetradecahydrobenzo[p][1]oxa[4,7,10,14]tetraazacycloheptadecine-13-carboxamide |
| 306 | (3R,9S,13S)-N-(2-(benzo[d][1,3]dioxol-5-yl)ethyl)-3-benzyl-9-isobutyl-16-methoxy-10-methyl-5,8,11,15- |
| | tetraoxo-7-(((S)-tetrahydro-2H-pyran-2-yl)methyl)-2,3,4,5,6,7,8,9,10,11,12,13,14,15- |
| | tetradecahydrobenzo[p][1]oxa[4,7,10,14]tetraazacycloheptadecine-13-carboxamide |
| 307 | (3R,9S,13S)-N-(2-(benzo[d][1,3]dioxol-5-yl)ethyl)-3-benzyl-9-(cyclopentylmethyl)-16-methoxy-10- |
| | methyl-5,8,11,15-tetraoxo-7-(((R)-tetrahydro-2H-pyran-2-yl)methyl)-2,3,4,5,6,7,8,9,10,11,12,13,14,15- |
| | tetradecahydrobenzo[p][1]oxa[4,7,10,14]tetraazacycloheptadecine-13-carboxamide |
| 308 | (3R,9S,13S)-N-(2-(benzo[d][1,3]dioxol-5-yl)ethyl)-3-benzyl-9-(cyclopentylmethyl)-16-methoxy-10- |
| | methyl-5,8,11,15-tetraoxo-7-(((S)-tetrahydro-2H-pyran-2-yl)methyl)-2,3,4,5,6,7,8,9,10,11,12,13,14,15- |
| | tetradecahydrobenzo[p][1]oxa[4,7,10,14]tetraazacycloheptadecine-13-carboxamide |
| 309 | (3R,9S,13S)-N-(2-(benzo[d][1,3]dioxol-5-yl)ethyl)-3-benzyl-9-(cyclohexylmethyl)-16-methoxy-10-methyl- |
| | 5,8,11,15-tetraoxo-7-(((S)-tetrahydro-2H-pyran-2-yl)methyl)-2,3,4,5,6,7,8,9,10,11,12,13,14,15- |
| | tetradecahydrobenzo[p][1]oxa[4,7,10,14]tetraazacycloheptadecine-13-carboxamide |
| 310 | (3R,9S,13S)-3-benzyl-9-isobutyl-16-methoxy-N-(3-methoxyphenethyl)-10-methyl-5,8,11,15-tetraoxo-7- |
| | (2-(RS)-tetrahydro-2H-pyran-2-yl)ethyl)-2,3,4,5,6,7,8,9,10,11,12,13,14,15- |
| | tetradecahydrobenzo[p][1]oxa[4,7,10,14]tetraazacycloheptadecine-13-carboxamide |
| 311 | (3R,9S,13S)-3-benzyl-9-isobutyl-16-methoxy-N-(3-methoxyphenethyl)-10-methyl-5,8,11,15-tetraoxo-7- |
| | (((R)-tetrahydro-2H-pyran-2-yl)methyl)-2,3,4,5,6,7,8,9,10,11,12,13,14,15- |
| | tetradecahydrobenzo[p][1]oxa[4,7,10,14]tetraazacycloheptadecine-13-carboxamide |
| 312 | (3R,9S,13S)-3-benzyl-9-isobutyl-16-methoxy-N-(3-methoxyphenethyl)-10-methyl-5,8,11,15-tetraoxo-7- |
| | (((S)-tetrahydro-2H-pyran-2-yl)methyl)-2,3,4,5,6,7,8,9,10,11,12,13,14,15- |
| | tetradecahydrobenzo[p][1]oxa[4,7,10,14]tetraazacycloheptadecine-13-carboxamide |
| 313 | (3R,9S,13S)-3-benzyl-9-(cyclopentylmethyl)-16-methoxy-N-(3-methoxyphenethyl)-10-methyl-5,8,11,15- |
| | tetraoxo-7-(((R)-tetrahydro-2H-pyran-2-yl)methyl)-2,3,4,5,6,7,8,9,10,11,12,13,14,15- |
| | tetradecahydrobenzo[p][1]oxa[4,7,10,14]tetraazacycloheptadecine-13-carboxamide |
| 314 | (3R,9S,13S)-3-benzyl-9-(cyclohexylmethyl)-16-methoxy-N-(3-methoxyphenethyl)-10-methyl-5,8,11,15- |
| | tetraoxo-7-(((S)-tetrahydro-2H-pyran-2-yl)methyl)-2,3,4,5,6,7,8,9,10,11,12,13,14,15- |
| | tetradecahydrobenzo[p][1]oxa[4,7,10,14]tetraazacycloheptadecine-13-carboxamide |
| 315 | (3S,7S,13R)-13-benzyl-9-(2-cyclohexylethyl)-7-isobutyl-N-(3-methoxyphenethyl)-6-methyl-1,5,8,11- |
| | tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 316 | (3S,7S,13R)-13-benzyl-9-(2-cyclohexylethyl)-7-isobutyl-N-(3-(methoxy-d3)phenethyl)-6-methyl-1,5,8,11- |
| | tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 317 | (3R,9S,13S)-3-benzyl-9-isobutyl-N-(3-methoxyphenethyl)-7,10-dimethyl-5,8,11,15-tetraoxo-16- |
| | (trifluoromethoxy)-2,3,4,5,6,7,8,9,10,11,12,13,14,15- |
| | tetradecahydrobenzo[p][1]oxa[4,7,10,14]tetraazacycloheptadecine-13-carboxamide |
| 318 | (3S,7S,13R)-N-(2-(benzo[d][1,3]dioxol-5-yl)ethyl)-13-benzyl-7-isobutyl-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[17,16- |
| | c]quinoline-3-carboxamide |
| 319 | (3S,7S,13R)-13-benzyl-7-isobutyl-N-(3-methoxyphenethyl)-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[17,16- |
| | c]quinoline-3-carboxamide |
| 320 | (3S,7S,13R)-13-benzyl-7-isobutyl-N-(3-(methoxy-d3)phenethyl)-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[17,16- |
| | c]quinoline-3-carboxamide |
| 321 | (8R,14S,18S)-N-(2-(benzo[d][1,3]dioxol-5-yl)ethyl)-8-benzyl-14-isobutyl-12,15-dimethyl-10,13,16,20- |
| | tetraoxo-7,8,9,10,11,12,13,14,15,16,17,18,19,20-tetradecahydro-[1,3]dioxolo[4′,5′:5,6]benzo[1,2- |
| | p][1]oxa[4,7,10,14]tetraazacycloheptadecine-18-carboxamide |
| 322 | (8R,14S,18S)-N-(2-(benzo[d][1,3]dioxol-5-yl)ethyl)-8-benzyl-14-isobutyl-12,15-dimethyl-10,13,16,20- |
| | tetraoxo-7,8,9,10,11,12,13,14,15,16,17,18,19,20-tetradecahydro-[1,3]dioxolo[4′,5′:5,6]benzo[1,2- |
| | p][1]oxa[4,7,10,14]tetraazacycloheptadecine-2,2-d2-18-carboxamide |
| 323 | (8R,14S,18S)-8-benzyl-14-isobutyl-N-(3-methoxyphenethyl)-12,15-dimethyl-10,13,16,20-tetraoxo- |
| | 7,8,9,10,11,12,13,14,15,16,17,18,19,20-tetradecahydro-[1,3]dioxolo[4′,5′:5,6]benzo[1,2- |
| | p][1]oxa[4,7,10,14]tetraazacycloheptadecine-18-carboxamide |
| 324 | (8R,14S,18S)-8-benzyl-14-isobutyl-N-(3-methoxyphenethyl)-12,15-dimethyl-10,13,16,20-tetraoxo- |
| | 7,8,9,10,11,12,13,14,15,16,17,18,19,20-tetradecahydro-[1,3]dioxolo[4′,5′:5,6]benzo[1,2- |
| | p][1]oxa[4,7,10,14]tetraazacycloheptadecine-2,2-d2-18-carboxamide |
| 325 | (4aR,7R,18S,22S)-7-benzyl-N-(3,5-dimethoxyphenethyl)-22-isobutyl-21-methyl-5,16,20,23-tetraoxo- |
| | 1,2,4,4a,5,6,7,8,16,17,18,19,20,21,22,23-hexadecahydronaphtho[1,2-p][1,4]oxazino[3,4- |
| | f][1]oxa[4,7,10,14]tetraazacycloheptadecine-18-carboxamide |
| 326 | (4aR,7R,18S,22S)-N-(2-(benzo[d][1,3]dioxol-5-yl)ethyl)-7-benzyl-22-isobutyl-21-methyl-5,16,20,23- |
| | tetraoxo-1,2,4,4a,5,6,7,8,16,17,18,19,20,21,22,23-hexadecahydronaphtho[1,2-p][1,4]oxazino[3,4- |
| | f][1]oxa[4,7,10,14]tetraazacycloheptadecine-18-carboxamide |
| 327 | (4aR,7R,18S,22S)-N-(2-(benzo[d]oxazol-2-yl)ethyl)-7-benzyl-22-isobutyl-21-methyl-5,16,20,23-tetraoxo- |
| | 1,2,4,4a,5,6,7,8,16,17,18,19,20,21,22,23-hexadecahydronaphtho[1,2-p][1,4]oxazino[3,4- |
| | f][1]oxa[4,7,10,14]tetraazacycloheptadecine-18-carboxamide |
| 328 | (8S,12S,18aR,21R)-21-benzyl-N-(3,5-dimethoxyphenethyl)-12-isobutyl-5,11-dimethyl-6,10,13,19- |
| | tetraoxo-6,7,8,9,10,11,12,13,16,17,18,18a,19,20,21,22-hexadecahydro-5H,15H- |
| | imidazo[4′,5′:5,6]benzo[1,2-p]pyrido[2,1-f][1]oxa[4,7,10,14]tetraazacycloheptadecine-8-carboxamide |
| 329 | (8S,12S,18aR,21R)-N-(2-(benzo[d][1,3]dioxol-5-yl)ethyl)-21-benzyl-12-isobutyl-5,11-dimethyl- |
| | 6,10,13,19-tetraoxo-6,7,8,9,10,11,12,13,16,17,18,18a,19,20,21,22-hexadecahydro-5H,15H- |
| | imidazo[4′,5′:5,6]benzo[1,2-p]pyrido[2,1-f][1]oxa[4,7,10,14]tetraazacycloheptadecine-8-carboxamide |
| 330 | (3S,7S,13R)-9-(2-(7-oxabicyclo[2.2.1]heptan-2-yl)ethyl)-N-(2-(benzo[d][1,3]dioxol-5-yl)ethyl)-13-benzyl- |
| | 7-isobutyl-6-methyl-1,5,8,11-tetraoxo-1,2,3,4,5,6,7,8,9,10,11,12,13,14-tetradecahydronaphtho[1,2- |
| | p][1]oxa[4,7,10,14]tetraazacycloheptadecine-3-carboxamide |
| 331 | (3S,7S,13R)-9-(2-(5-oxaspiro[3.4]octan-6-yl)ethyl)-N-(2-(benzo[d][1,3]dioxol-5-yl)ethyl)-13-benzyl-7- |
| | isobutyl-6-methyl-1,5,8,11-tetraoxo-1,2,3,4,5,6,7,8,9,10,11,12,13,14-tetradecahydronaphtho[1,2- |
| | p][1]oxa[4,7,10,14]tetraazacycloheptadecine-3-carboxamide |
| 332 | (3S,7S,13R)-N-(2-(benzo[d][1,3]dioxol-5-yl)ethyl)-13-benzyl-7-isobutyl-6-methyl-1,5,8,11-tetraoxo-9-(2- |
| | (tetrahydrofuran-2-yl)ethyl)-1,2,3,4,5,6,7,8,9,10,11,12,13,14-tetradecahydronaphtho[1,2- |
| | p][1]oxa[4,7,10,14]tetraazacycloheptadecine-3-carboxamide |
| 333 | (3S,7S,13R)-N-(2-(benzo[d][1,3]dioxol-5-yl)ethyl)-13-benzyl-7-isobutyl-6-methyl-1,5,8,11-tetraoxo-9-(2- |
| | (tetrahydro-2H-pyran-3-yl)ethyl)-1,2,3,4,5,6,7,8,9,10,11,12,13,14-tetradecahydronaphtho[1,2- |
| | p][1]oxa[4,7,10,14]tetraazacycloheptadecine-3-carboxamide |
| 334 | (3S,7S,13R)-9-(2-(2,9-dioxaspiro[5.5]undecan-3-yl)ethyl)-N-(2-(benzo[d][1,3]dioxol-5-yl)ethyl)-13- |
| | benzyl-7-isobutyl-6-methyl-1,5,8,11-tetraoxo-1,2,3,4,5,6,7,8,9,10,11,12,13,14- |
| | tetradecahydronaphtho[1,2-p][1]oxa[4,7,10,14]tetraazacycloheptadecine-3-carboxamide |
| 335 | (3S,7S,13R)-N-(2-(benzo[d][1,3]dioxol-5-yl)ethyl)-13-benzyl-9-(2-(tert-butoxy)ethyl)-7-isobutyl-6-methyl- |
| | 1,5,8,11-tetraoxo-1,2,3,4,5,6,7,8,9,10,11,12,13,14-tetradecahydronaphtho[1,2- |
| | p][1]oxa[4,7,10,14]tetraazacycloheptadecine-3-carboxamide |
| 336 | (3S,7S,13R)-N-(2-(benzo[d][1,3]dioxol-5-yl)ethyl)-13-benzyl-7-isobutyl-9-(2-(3-methoxycyclobutyl)ethyl)- |
| | 6-methyl-1,5,8,11-tetraoxo-1,2,3,4,5,6,7,8,9,10,11,12,13,14-tetradecahydronaphtho[1,2- |
| | p][1]oxa[4,7,10,14]tetraazacycloheptadecine-3-carboxamide |
| 337 | (3S,7S,13R)-N-(2-(benzo[d][1,3]dioxol-5-yl)ethyl)-13-benzyl-7-(cyclobutylmethyl)-6-methyl-1,5,8,11- |
| | tetraoxo-9-(((S)-tetrahydro-2H-pyran-2-yl)methyl)-1,2,3,4,5,6,7,8,9,10,11,12,13,14- |
| | tetradecahydronaphtho[1,2-p][1]oxa[4,7,10,14]tetraazacycloheptadecine-3-carboxamide |
| 338 | (3S,7S,13R)-N-(2-(benzo[d][1,3]dioxol-5-yl)ethyl)-13-benzyl-7-(cyclobutylmethyl)-6-methyl-1,5,8,11- |
| | tetraoxo-9-(((R)-tetrahydro-2H-pyran-2-yl)methyl)-1,2,3,4,5,6,7,8,9,10,11,12,13,14- |
| | tetradecahydronaphtho[1,2-p][1]oxa[4,7,10,14]tetraazacycloheptadecine-3-carboxamide |
| 339 | (3S,7S,13R)-N-(2-(benzo[d][1,3]dioxol-5-yl)ethyl)-13-benzyl-9-(2-cyclohexylethyl)-6-methyl-1,5,8,11- |
| | tetraoxo-7-(prop-2-yn-1-yl)-1,2,3,4,5,6,7,8,9,10,11,12,13,14-tetradecahydronaphtho[1,2- |
| | p][1]oxa[4,7,10,14]tetraazacycloheptadecine-3-carboxamide |
| 340 | (3S,7S,13R)-N-(2-(benzo[d][1,3]dioxol-5-yl)ethyl)-13-benzyl-7-(cyclopropylmethyl)-6-methyl-1,5,8,11- |
| | tetraoxo-9-(((R)-tetrahydro-2H-pyran-2-yl)methyl)-1,2,3,4,5,6,7,8,9,10,11,12,13,14- |
| | tetradecahydronaphtho[1,2-p][1]oxa[4,7,10,14]tetraazacycloheptadecine-3-carboxamide |
| 341 | (3S,7S,13R)-N-(2-(benzo[d][1,3]dioxol-5-yl)ethyl)-13-benzyl-9-(2-cyclohexylethyl)-7-(cyclopropylmethyl)- |
| | 6-methyl-1,5,8,11-tetraoxo-1,2,3,4,5,6,7,8,9,10,11,12,13,14-tetradecahydronaphtho[1,2- |
| | p][1]oxa[4,7,10,14]tetraazacycloheptadecine-3-carboxamide |
| 342 | (8S,12S,18aS,21R)-21-benzyl-12-isobutyl-N-(3-methoxyphenethyl)-11-methyl-6,10,13,19-tetraoxo- |
| | 6,7,8,9,10,11,12,13,16,17,18,18a,19,20,21,22-hexadecahydro-15H-oxazolo[4′,5′:5,6]benzo[1,2- |
| | p]pyrido[2,1-f][1]oxa[4,7,10,14]tetraazacycloheptadecine-8-carboxamide |
| 343 | (8S,12S,18aR,21R)-21-benzyl-12-isobutyl-N-(3-methoxyphenethyl)-11-methyl-6,10,13,19-tetraoxo- |
| | 6,7,8,9,10,11,12,13,16,17,18,18a,19,20,21,22-hexadecahydro-15H-oxazolo[4′,5′:5,6]benzo[1,2- |
| | p]pyrido[2,1-f][1]oxa[4,7,10,14]tetraazacycloheptadecine-8-carboxamide |
| 344 | (3S,7S,13R)-13-benzyl-9-(2-cyclohexylethyl)-7-isobutyl-6-methyl-N-(2-(3-methylisoxazol-5-yl)ethyl)- |
| | 1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 345 | (8R,14S,18S)-N-(2-(benzo[d][1,3]dioxol-5-yl)ethyl)-8-benzyl-12-(2-cyclohexylethyl)-14-isobutyl-15- |
| | methyl-10,13,16,20-tetraoxo-7,8,9,10,11,12,13,14,15,16,17,18,19,20-tetradecahydro- |
| | [1,3]dioxolo[4′,5′:5,6]benzo[1,2-p][1]oxa[4,7,10,14]tetraazacycloheptadecine-18-carboxamide |
| 346 | (8R,14S,18S)-8-benzyl-12-(2-cyclohexylethyl)-14-isobutyl-N-(3-methoxyphenethyl)-15-methyl- |
| | 10,13,16,20-tetraoxo-7,8,9,10,11,12,13,14,15,16,17,18,19,20-tetradecahydro- |
| | [1,3]dioxolo[4′,5′:5,6]benzo[1,2-p][1]oxa[4,7,10,14]tetraazacycloheptadecine-18-carboxamide |
| 347 | (8R,14S,18S)-8-benzyl-12-(2-cyclohexylethyl)-14-isobutyl-N-(3-(methoxy-d3)phenethyl)-15-methyl- |
| | 10,13,16,20-tetraoxo-7,8,9,10,11,12,13,14,15,16,17,18,19,20-tetradecahydro- |
| | [1,3]dioxolo[4′,5′:5,6]benzo[1,2-p][1]oxa[4,7,10,14]tetraazacycloheptadecine-18-carboxamide |
| 348 | (8S,12S,18aR,21R)-21-benzyl-17-cyclopropyl-12-isobutyl-N-(3-methoxyphenethyl)-5,11-dimethyl- |
| | 6,10,13,19-tetraoxo-6,7,8,9,10,11,12,13,16,17,18,18a,19,20,21,22-hexadecahydro-5H,15H- |
| | imidazo[4′,5′:5,6]benzo[1,2-p]pyrazino[2,1-f][1]oxa[4,7,10,14]tetraazacycloheptadecine-8-carboxamide |
| 349 | (3S,7S,13R)-N-(2-(benzo[d][1,3]dioxol-5-yl)ethyl)-13-benzyl-18-fluoro-7-isobutyl-6,9-dimethyl-1,5,8,11- |
| | tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 350 | (3S,7S,13R)-13-benzyl-18-fluoro-7-isobutyl-N-(3-methoxyphenethyl)-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[17,16- |
| | c]quinoline-3-carboxamide |
| 351 | (8S,12S,18aR,21R)-21-benzyl-12-isobutyl-N-(3-methoxyphenethyl)-4,11-dimethyl-6,10,13,19-tetraoxo- |
| | 6,7,8,9,10,11,12,13,16,17,18,18a,19,20,21,22-hexadecahydro-15H-oxazolo[5′,4′:5,6]benzo[1,2- |
| | p]pyrido[2,1-f][1]oxa[4,7,10,14]tetraazacycloheptadecine-8-carboxamide |
| 352 | (8S,12S,18aR,21R)-21-benzyl-12-isobutyl-17-isopropyl-N-(3-methoxyphenethyl)-4,11-dimethyl- |
| | 6,10,13,19-tetraoxo-6,7,8,9,10,11,12,13,16,17,18,18a,19,20,21,22-hexadecahydro-15H- |
| | oxazolo[4′,5′:5,6]benzo[1,2-p]pyrazino[2,1-f][1]oxa[4,7,10,14]tetraazacycloheptadecine-8-carboxamide |
| 353 | (8S,12S,18aR,21R)-21-benzyl-12-isobutyl-17-isopropyl-N-(3-methoxyphenethyl)-5,11-dimethyl- |
| | 6,10,13,19-tetraoxo-6,7,8,9,10,11,12,13,16,17,18,18a,19,20,21,22-hexadecahydro-5H,15H- |
| | imidazo[4′,5′:5,6]benzo[1,2-p]pyrazino[2,1-f][1]oxa[4,7,10,14]tetraazacycloheptadecine-8-carboxamide |
| 354 | (4aR,7R,18S,22S)-7-benzyl-22-isobutyl-3-(2-methoxyethyl)-N-(3-methoxyphenethyl)-21-methyl- |
| | 5,16,20,23-tetraoxo-2,3,4,4a,5,6,7,8,16,17,18,19,20,21,22,23-hexadecahydro-1H-naphtho[1,2- |
| | p]pyrazino[2,1-f][1]oxa[4,7,10,14]tetraazacycloheptadecine-18-carboxamide |
| 355 | (4aR,7R,18S,22S)-7-benzyl-3-cyclobutyl-22-isobutyl-N-(3-methoxyphenethyl)-21-methyl-5,16,20,23- |
| | tetraoxo-2,3,4,4a,5,6,7,8,16,17,18,19,20,21,22,23-hexadecahydro-1H-naphtho[1,2-p]pyrazino[2,1- |
| | f][1]oxa[4,7,10,14]tetraazacycloheptadecine-18-carboxamide |
| 356 | (4aR,7R,18S,22S)-7-benzyl-3-cyclopropyl-22-isobutyl-N-(3-methoxyphenethyl)-21-methyl-5,16,20,23- |
| | tetraoxo-2,3,4,4a,5,6,7,8,16,17,18,19,20,21,22,23-hexadecahydro-1H-naphtho[1,2-p]pyrazino[2,1- |
| | f][1]oxa[4,7,10,14]tetraazacycloheptadecine-18-carboxamide |
| 357 | (4aS,7R,18S,22S)-7-benzyl-3-cyclopropyl-22-isobutyl-N-(3-methoxyphenethyl)-21-methyl-5,16,20,23- |
| | tetraoxo-2,3,4,4a,5,6,7,8,16,17,18,19,20,21,22,23-hexadecahydro-1H-naphtho[1,2-p]pyrazino[2,1- |
| | f][1]oxa[4,7,10,14]tetraazacycloheptadecine-18-carboxamide |
| 358 | (3S,7S,13R)-N-(2-(benzo[d][1,3]dioxol-5-yl)ethyl)-13-benzyl-7-(cyclopropylmethyl)-6-methyl-1,5,8,11- |
| | tetraoxo-9-(2-((S)-tetrahydro-2H-pyran-2-yl)ethyl)-1,2,3,4,5,6,7,8,9,10,11,12,13,14- |
| | tetradecahydronaphtho[1,2-p][1]oxa[4,7,10,14]tetraazacycloheptadecine-3-carboxamide |
| 359 | (3S,7S,13R)-N-(2-(benzo[d][1,3]dioxol-5-yl)ethyl)-13-benzyl-7-(cyclopropylmethyl)-6-methyl-1,5,8,11- |
| | tetraoxo-9-(2-((R)-tetrahydro-2H-pyran-2-yl)ethyl)-1,2,3,4,5,6,7,8,9,10,11,12,13,14- |
| | tetradecahydronaphtho[1,2-p][1]oxa[4,7,10,14]tetraazacycloheptadecine-3-carboxamide |
| 360 | (3S,7S,13R)-N-(2-(benzo[d][1,3]dioxol-5-yl)ethyl)-13-benzyl-7-(cyclobutylmethyl)-6-methyl-1,5,8,11- |
| | tetraoxo-9-(2-((S)-tetrahydro-2H-pyran-2-yl)ethyl)-1,2,3,4,5,6,7,8,9,10,11,12,13,14- |
| | tetradecahydronaphtho[1,2-p][1]oxa[4,7,10,14]tetraazacycloheptadecine-3-carboxamide |
| 361 | (3S,7S,13R)-N-(2-(benzo[d][1,3]dioxol-5-yl)ethyl)-13-benzyl-7-(cyclobutylmethyl)-6-methyl-1,5,8,11- |
| | tetraoxo-9-(2-((R)-tetrahydro-2H-pyran-2-yl)ethyl)-1,2,3,4,5,6,7,8,9,10,11,12,13,14- |
| | tetradecahydronaphtho[1,2-p][1]oxa[4,7,10,14]tetraazacycloheptadecine-3-carboxamide |
| 362 | (3S,7S,13R)-N-(2-(benzo[d][1,3]dioxol-5-yl)ethyl)-13-benzyl-7-isobutyl-6-methyl-1,5,8,11-tetraoxo-9-(2- |
| | ((S)-tetrahydro-2H-pyran-2-yl)ethyl)-1,2,3,4,5,6,7,8,9,10,11,12,13,14-tetradecahydronaphtho[1,2- |
| | p][1]oxa[4,7,10,14]tetraazacycloheptadecine-3-carboxamide |
| 363 | (3S,7S,13R)-N-(2-(benzo[d][1,3]dioxol-5-yl)ethyl)-13-benzyl-7-isobutyl-6-methyl-1,5,8,11-tetraoxo-9-(2- |
| | (R)-tetrahydro-2H-pyran-2-yl)ethyl)-1,2,3,4,5,6,7,8,9,10,11,12,13,14-tetradecahydronaphtho[1,2- |
| | p][1]oxa[4,7,10,14]tetraazacycloheptadecine-3-carboxamide |
| 364 | (8S,12S,18aR,21R)-21-benzyl-12,17-diisobutyl-N-(3-methoxyphenethyl)-5,11-dimethyl-6,10,13,19- |
| | tetraoxo-6,7,8,9,10,11,12,13,16,17,18,18a,19,20,21,22-hexadecahydro-5H,15H- |
| | imidazo[4′,5′:5,6]benzo[1,2-p]pyrazino[2,1-f][1]oxa[4,7,10,14]tetraazacycloheptadecine-8-carboxamide |
| 365 | (9S,13S,19aR,22R)-22-benzyl-13-isobutyl-N-(3-methoxyphenethyl)-12-methyl-7,11,14,20-tetraoxo- |
| | 7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 366 | (9S,13S,19aS,22R)-22-benzyl-13-isobutyl-N-(3-methoxyphenethyl)-12-methyl-7,11,14,20-tetraoxo- |
| | 7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 367 | (9S,13S,19aR,22R)-22-benzyl-13-isobutyl-12-methyl-N-(2-(3-methylisoxazol-5-yl)ethyl)-7,11,14,20- |
| | tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 368 | (8S,12S,18aR,21R)-21-benzyl-N-(3,5-dimethoxyphenethyl)-12-isobutyl-4,11-dimethyl-6,10,13,19- |
| | tetraoxo-6,7,8,9,10,11,12,13,16,17,18,18a,19,20,21,22-hexadecahydro-15H- |
| | oxazolo[5′,4′:5,6]benzo[1,2-p]pyrido[2,1-f][1]oxa[4,7,10,14]tetraazacycloheptadecine-8-carboxamide |
| 369 | 2,2,2-trichloroethyl (4aR,7R,18S,22S)-7-benzyl-22-isobutyl-18-((3-methoxyphenethyl)carbamoyl)-21- |
| | methyl-5,16,20,23-tetraoxo-1,2,4,4a,5,6,7,8,16,17,18,19,20,21,22,23-hexadecahydro-3H-naphtho[1,2- |
| | p]pyrazino[2,1-f][1]oxa[4,7,10,14]tetraazacycloheptadecine-3-carboxylate |
| 370 | (4aR,7R,18S,22S)-7-benzyl-N-(2-(5,6-dihydro-4H-cyclopenta[d]thiazol-2-yl)ethyl)-22-isobutyl-21-methyl- |
| | 5,16,20,23-tetraoxo-1,2,4,4a,5,6,7,8,16,17,18,19,20,21,22,23-hexadecahydronaphtho[1,2- |
| | p][1,4]oxazino[3,4-f][1]oxa[4,7,10,14]tetraazacycloheptadecine-18-carboxamide |
| 371 | (4aR,7R,18S,22S)-7-benzyl-N-(3-(difluoromethoxy)phenethyl)-22-isobutyl-21-methyl-5,16,20,23- |
| | tetraoxo-1,2,4,4a,5,6,7,8,16,17,18,19,20,21,22,23-hexadecahydronaphtho[1,2-p][1,4]oxazino[3,4- |
| | f][1]oxa[4,7,10,14]tetraazacycloheptadecine-18-carboxamide |
| 372 | (9S,13S,19aR,22R)-22-benzyl-13,18-diisobutyl-N-(3-methoxyphenethyl)-12-methyl-7,11,14,20-tetraoxo- |
| | 7,8,9,10,11,12,13,14,17,18,19,19a,20,21,22,23-hexadecahydro-16H- |
| | pyrazino[2′,11:6,7][1]oxa[4,7,10,14]tetraazacycloheptadecino[17,16-c]quinoline-9-carboxamide |
| 373 | (9S,13S,19aS,22R)-22-benzyl-13,18-diisobutyl-N-(3-methoxyphenethyl)-12-methyl-7,11,14,20-tetraoxo- |
| | 7,8,9,10,11,12,13,14,17,18,19,19a,20,21,22,23-hexadecahydro-16H- |
| | pyrazino[2′,11:6,7][1]oxa[4,7,10,14]tetraazacycloheptadecino[17,16-c]quinoline-9-carboxamide |
| 374 | (9S,13S,19aR,22R)-22-benzyl-13-isobutyl-18-isopropyl-N-(3-methoxyphenethyl)-12-methyl-7,11,14,20- |
| | tetraoxo-7,8,9,10,11,12,13,14,17,18,19,19a,20,21,22,23-hexadecahydro-16H- |
| | pyrazino[2′,1′:6,7][1]oxa[4,7,10,14]tetraazacycloheptadecino[17,16-c]quinoline-9-carboxamide |
| 375 | (9S,13S,19aR,22R)-22-benzyl-13-isobutyl-18-isopropyl-12-methyl-N-(2-(3-methylisoxazol-5-yl)ethyl)- |
| | 7,11,14,20-tetraoxo-7,8,9,10,11,12,13,14,17,18,19,19a,20,21,22,23-hexadecahydro-16H- |
| | pyrazino[2′,1′:6,7][1]oxa[4,7,10,14]tetraazacycloheptadecino[17,16-c]quinoline-9-carboxamide |
| 376 | (4aR,7R,18S,22S)-7-benzyl-22-isobutyl-N-(3-methoxyphenethyl)-21-methyl-5,16,20,23-tetraoxo- |
| | 4,4a,5,6,7,8,16,17,18,19,20,21,22,23-tetradecahydro-1H-naphtho[1,2-p]pyrido[2,1- |
| | f][1]oxa[4,7,10,14]tetraazacycloheptadecine-18-carboxamide |
| 377 | (8S,12S,18aR,21R)-21-benzyl-N-(3,5-dimethoxyphenethyl)-12-isobutyl-4,5,11-trimethyl-6,10,13,19- |
| | tetraoxo-6,7,8,9,10,11,12,13,16,17,18,18a,19,20,21,22-hexadecahydro-5H,15H- |
| | imidazo[4′,5′:5,6]benzo[1,2-p]pyrido[2,1-f][1]oxa[4,7,10,14]tetraazacycloheptadecine-8-carboxamide |
| 378 | (4aR,7R,18S,22S)-7-benzyl-N-(3-bromophenethyl)-22-isobutyl-21-methyl-5,16,20,23-tetraoxo- |
| | 1,2,4,4a,5,6,7,8,16,17,18,19,20,21,22,23-hexadecahydronaphtho[1,2-p][1,4]oxazino[3,4- |
| | f][1]oxa[4,7,10,14]tetraazacycloheptadecine-18-carboxamide |
| 379 | (8R,14S,18S)-N-(2-(benzo[d][1,3]dioxol-5-yl)ethyl)-8-benzyl-12-(2-cyclohexylethyl)-14-isobutyl-2,15- |
| | dimethyl-10,13,16,20-tetraoxo-7,8,9,10,11,12,13,14,15,16,17,18,19,20- |
| | tetradecahydrooxazolo[4′,5′:5,6]benzo[1,2-p][1]oxa[4,7,10,14]tetraazacycloheptadecine-18- |
| | carboxamide |
| 380 | (8R,14S,18S)-8-benzyl-12-(2-cyclohexylethyl)-14-isobutyl-N-(3-methoxyphenethyl)-2,15-dimethyl- |
| | 10,13,16,20-tetraoxo-7,8,9,10,11,12,13,14,15,16,17,18,19,20- |
| | tetradecahydrooxazolo[4′,5′:5,6]benzo[1,2-p][1]oxa[4,7,10,14]tetraazacycloheptadecine-18- |
| | carboxamide |
| 381 | (8R,14S,18S)-8-benzyl-12-(2-cyclohexylethyl)-14-isobutyl-N-(3-(methoxy-d3)phenethyl)-2,15-dimethyl- |
| | 10,13,16,20-tetraoxo-7,8,9,10,11,12,13,14,15,16,17,18,19,20- |
| | tetradecahydrooxazolo[4′,5′:5,6]benzo[1,2-p][1]oxa[4,7,10,14]tetraazacycloheptadecine-18- |
| | carboxamide |
| 382 | (3S,7S,13R)-N-(2-(benzo[d][1,3]dioxol-5-yl)ethyl)-13-benzyl-9-(2-cyclohexylethyl)-18-fluoro-7-isobutyl-6- |
| | methyl-1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 383 | (3S,7S,13R)-13-benzyl-9-(2-cyclohexylethyl)-18-fluoro-7-isobutyl-N-(3-methoxyphenethyl)-6-methyl- |
| | 1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 384 | (3S,7S,13R)-13-benzyl-9-(2-cyclohexylethyl)-18-fluoro-7-isobutyl-N-(3-(methoxy-d3)phenethyl)-6- |
| | methyl-1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 385 | (3′S,7′S,13′R)-13′-benzyl-7′-isobutyl-N-(3-methoxyphenethyl)-6′,9′-dimethyl-1′,5′,8′,11′-tetraoxo- |
| | 2′,3′,4′,5′,6′,7′,8′,9′,11′,12′,13′,14′-dodecahydro-1′H-spiro[cyclopropane-1,10′-naphtho[1,2- |
| | p][1]oxa[4,7,10,14]tetraazacycloheptadecine]-3′-carboxamide |
| 386 | (3′S,7′S,13′R)-13′-benzyl-7′-isobutyl-N-(3-methoxyphenethyl)-6′-methyl-1′,5′,8′,11′-tetraoxo- |
| | 2′,3′,4′,5′,6′,7′,8′,9′,11′,12′,13′,14′-dodecahydro-1′H-spiro[cyclopropane-1,10′-naphtho[1,2- |
| | p][1]oxa[4,7,10,14]tetraazacycloheptadecine]-3′-carboxamide |
| 387 | (4aR,7R,18S,22S)-N-(4-(2H-1,2,3-triazol-2-yl)phenethyl)-7-benzyl-22-isobutyl-21-methyl-5,16,20,23- |
| | tetraoxo-1,2,4,4a,5,6,7,8,16,17,18,19,20,21,22,23-hexadecahydronaphtho[1,2-p][1,4]oxazino[3,4- |
| | f][1]oxa[4,7,10,14]tetraazacycloheptadecine-18-carboxamide |
| 388 | (9S,13S,19aS,22R)-22-benzyl-18-cyclopropyl-13-isobutyl-N-(3-methoxyphenethyl)-12-methyl- |
| | 7,11,14,20-tetraoxo-7,8,9,10,11,12,13,14,17,18,19,19a,20,21,22,23-hexadecahydro-16H- |
| | pyrazino[2′,11:6,7][1]oxa[4,7,10,14]tetraazacycloheptadecino[17,16-c]quinoline-9-carboxamide |
| 389 | (9S,13S,19aR,22R)-22-benzyl-18-cyclopropyl-13-isobutyl-N-(3-methoxyphenethyl)-12-methyl- |
| | 7,11,14,20-tetraoxo-7,8,9,10,11,12,13,14,17,18,19,19a,20,21,22,23-hexadecahydro-16H- |
| | pyrazino[2′,1′:6,7][1]oxa[4,7,10,14]tetraazacycloheptadecino[17,16-c]quinoline-9-carboxamide |
| 390 | (9S,13S,19aS,22R)-22-benzyl-18-cyclobutyl-13-isobutyl-N-(3-methoxyphenethyl)-12-methyl-7,11,14,20- |
| | tetraoxo-7,8,9,10,11,12,13,14,17,18,19,19a,20,21,22,23-hexadecahydro-16H- |
| | pyrazino[2′,11:6,7][1]oxa[4,7,10,14]tetraazacycloheptadecino[17,16-c]quinoline-9-carboxamide |
| 391 | (9S,13S,19aR,22R)-22-benzyl-18-cyclobutyl-13-isobutyl-N-(3-methoxyphenethyl)-12-methyl-7,11,14,20- |
| | tetraoxo-7,8,9,10,11,12,13,14,17,18,19,19a,20,21,22,23-hexadecahydro-16H- |
| | pyrazino[2′,1′:6,7][1]oxa[4,7,10,14]tetraazacycloheptadecino[17,16-c]quinoline-9-carboxamide |
| 392 | (9S,13S,19aS,22R)-N-(2-(benzo[d][1,3]dioxol-5-yl)ethyl)-22-benzyl-18-cyclopropyl-13-isobutyl-12- |
| | methyl-7,11,14,20-tetraoxo-7,8,9,10,11,12,13,14,17,18,19,19a,20,21,22,23-hexadecahydro-16H- |
| | pyrazino[2′,1′:6,7][1]oxa[4,7,10,14]tetraazacycloheptadecino[17,16-c]quinoline-9-carboxamide |
| 393 | (9S,13S,19aR,22R)-N-(2-(benzo[d][1,3]dioxol-5-yl)ethyl)-22-benzyl-18-cyclopropyl-13-isobutyl-12- |
| | methyl-7,11,14,20-tetraoxo-7,8,9,10,11,12,13,14,17,18,19,19a,20,21,22,23-hexadecahydro-16H- |
| | pyrazino[2′,11:6,7][1]oxa[4,7,10,14]tetraazacycloheptadecino[17,16-c]quinoline-9-carboxamide |
| 394 | (9S,13S,19aS,22R)-N-(2-(benzo[d][1,3]dioxol-5-yl)ethyl)-22-benzyl-18-cyclobutyl-13-isobutyl-12-methyl- |
| | 7,11,14,20-tetraoxo-7,8,9,10,11,12,13,14,17,18,19,19a,20,21,22,23-hexadecahydro-16H- |
| | pyrazino[2′,1′:6,7][1]oxa[4,7,10,14]tetraazacycloheptadecino[17,16-c]quinoline-9-carboxamide |
| 395 | (9S,13S,19aR,22R)-N-(2-(benzo[d][1,3]dioxol-5-yl)ethyl)-22-benzyl-18-cyclobutyl-13-isobutyl-12-methyl- |
| | 7,11,14,20-tetraoxo-7,8,9,10,11,12,13,14,17,18,19,19a,20,21,22,23-hexadecahydro-16H- |
| | pyrazino[2′,11:6,7][1]oxa[4,7,10,14]tetraazacycloheptadecino[17,16-c]quinoline-9-carboxamide |
| 396 | (3′S,7′S,13′R)-13′-benzyl-7′-isobutyl-N-(3-methoxyphenethyl)-9′-(3-methoxypropyl)-6′-methyl-1′,5′,8′,11′- |
| | tetraoxo-2′,3′,4′,5′,6′,7′,8′,9′,11′,12′,13′,14′-dodecahydro-1′H-spiro[cyclopropane-1,10′-naphtho[1,2- |
| | p][1]oxa[4,7,10,14]tetraazacycloheptadecine]-3′-carboxamide |
| 397 | (4aR,7R,18S,22S)-7-benzyl-N-(3-ethoxyphenethyl)-22-isobutyl-21-methyl-5,16,20,23-tetraoxo- |
| | 1,2,4,4a,5,6,7,8,16,17,18,19,20,21,22,23-hexadecahydronaphtho[1,2-p][1,4]oxazino[3,4- |
| | f][1]oxa[4,7,10,14]tetraazacycloheptadecine-18-carboxamide |
| 398 | (9S,13S,19aR,22R)-22-benzyl-18-cyclopropyl-N-(3-(difluoromethoxy)phenethyl)-13-isobutyl-12-methyl- |
| | 7,11,14,20-tetraoxo-7,8,9,10,11,12,13,14,17,18,19,19a,20,21,22,23-hexadecahydro-16H- |
| | pyrazino[2′,11:6,7][1]oxa[4,7,10,14]tetraazacycloheptadecino[17,16-c]quinoline-9-carboxamide |
| 399 | (9S,13S,19aS,22R)-22-benzyl-18-cyclopropyl-N-(3-(difluoromethoxy)phenethyl)-13-isobutyl-12-methyl- |
| | 7,11,14,20-tetraoxo-7,8,9,10,11,12,13,14,17,18,19,19a,20,21,22,23-hexadecahydro-16H- |
| | pyrazino[2′,1′:6,7][1]oxa[4,7,10,14]tetraazacycloheptadecino[17,16-c]quinoline-9-carboxamide |
| 400 | (9S,13S,19aR,22R)-22-benzyl-18-cyclopropyl-N-(3-ethoxyphenethyl)-13-isobutyl-12-methyl-7,11,14,20- |
| | tetraoxo-7,8,9,10,11,12,13,14,17,18,19,19a,20,21,22,23-hexadecahydro-16H- |
| | pyrazino[2′,11:6,7][1]oxa[4,7,10,14]tetraazacycloheptadecino[17,16-c]quinoline-9-carboxamide |
| 401 | (9S,13S,19aS,22R)-22-benzyl-18-cyclopropyl-N-(3-ethoxyphenethyl)-13-isobutyl-12-methyl-7,11,14,20- |
| | tetraoxo-7,8,9,10,11,12,13,14,17,18,19,19a,20,21,22,23-hexadecahydro-16H- |
| | pyrazino[2′,1′:6,7][1]oxa[4,7,10,14]tetraazacycloheptadecino[17,16-c]quinoline-9-carboxamide |
| 402 | (9S,13S,19aR,22R)-22-benzyl-18-cyclopropyl-13-isobutyl-12-methyl-N-(2-(3-methylisoxazol-5-yl)ethyl)- |
| | 7,11,14,20-tetraoxo-7,8,9,10,11,12,13,14,17,18,19,19a,20,21,22,23-hexadecahydro-16H- |
| | pyrazino[2′,11:6,7][1]oxa[4,7,10,14]tetraazacycloheptadecino[17,16-c]quinoline-9-carboxamide |
| 403 | (9S,13S,19aS,22R)-22-benzyl-18-cyclopropyl-13-isobutyl-12-methyl-N-(2-(3-methylisoxazol-5-yl)ethyl)- |
| | 7,11,14,20-tetraoxo-7,8,9,10,11,12,13,14,17,18,19,19a,20,21,22,23-hexadecahydro-16H- |
| | pyrazino[2′,11:6,7][1]oxa[4,7,10,14]tetraazacycloheptadecino[17,16-c]quinoline-9-carboxamide |
| 404 | (9S,13S,19aR,22R)-22-benzyl-18-cyclopropyl-N-(2-(5,6-dihydro-4H-cyclopenta[d]thiazol-2-yl)ethyl)-13- |
| | isobutyl-12-methyl-7,11,14,20-tetraoxo-7,8,9,10,11,12,13,14,17,18,19,19a,20,21,22,23-hexadecahydro- |
| | 16H-pyrazino[2′,11:6,7][1]oxa[4,7,10,14]tetraazacycloheptadecino[17,16-c]quinoline-9-carboxamide |
| 405 | (9S,13S,19aS,22R)-22-benzyl-18-cyclopropyl-N-(2-(5,6-dihydro-4H-cyclopenta[d]thiazol-2-yl)ethyl)-13- |
| | isobutyl-12-methyl-7,11,14,20-tetraoxo-7,8,9,10,11,12,13,14,17,18,19,19a,20,21,22,23-hexadecahydro- |
| | 16H-pyrazino[2′,1′:6,7][1]oxa[4,7,10,14]tetraazacycloheptadecino[17,16-c]quinoline-9-carboxamide |
| 406 | (7S,11S,21R,23aR)-21-benzyl-7-isobutyl-N-(3-methoxyphenethyl)-8,16-dimethyl-6,9,13,23-tetraoxo- |
| | 1,3,4,6,7,8,9,10,11,12,13,20,21,22,23,23a-hexadecahydro-2H-oxazolo[5′,4′:4,5]benzo[1,2-p]pyrido[2,1- |
| | f][1]oxa[4,7,10,14]tetraazacycloheptadecine-11-carboxamide |
| 407 | (7S,11S,21R,23aR)-21-benzyl-7-isobutyl-N-(3-methoxyphenethyl)-8,16-dimethyl-6,9,13,23-tetraoxo- |
| | 1,3,4,6,7,8,9,10,11,12,13,20,21,22,23,23a-hexadecahydro-[1,4]oxazino[3,4- |
| | floxazolo[5′,4′:4,5]benzo[1,2-p][1]oxa[4,7,10,14]tetraazacycloheptadecine-11-carboxamide |
| 408 | (8R,14S,18S)-N-(2-(benzo[d][1,3]dioxol-5-yl)ethyl)-8-benzyl-2,2-difluoro-14-isobutyl-12,15-dimethyl- |
| | 10,13,16,20-tetraoxo-7,8,9,10,11,12,13,14,15,16,17,18,19,20-tetradecahydro- |
| | [1,3]dioxolo[4′,5′:5,6]benzo[1,2-p][1]oxa[4,7,10,14]tetraazacycloheptadecine-18-carboxamide |
| 409 | (8R,14S,18S)-8-benzyl-2,2-difluoro-14-isobutyl-N-(3-methoxyphenethyl)-12,15-dimethyl-10,13,16,20- |
| | tetraoxo-7,8,9,10,11,12,13,14,15,16,17,18,19,20-tetradecahydro-[1,3]dioxolo[4′,5′:5,6]benzo[1,2- |
| | p][1]oxa[4,7,10,14]tetraazacycloheptadecine-18-carboxamide |
| 410 | (8R,14S,18S)-8-benzyl-12-(2-cyclohexylethyl)-14-isobutyl-2,15-dimethyl-N-(2-(3-methylisoxazol-5- |
| | yl)ethyl)-10,13,16,20-tetraoxo-7,8,9,10,11,12,13,14,15,16,17,18,19,20- |
| | tetradecahydrooxazolo[4′,5′:5,6]benzo[1,2-p][1]oxa[4,7,10,14]tetraazacycloheptadecine-18- |
| | carboxamide |
| 411 | (3S,7S,13R)-13-benzyl-9-(2-cyclohexylethyl)-18-fluoro-7-isobutyl-6-methyl-N-(2-(3-methylisoxazol-5- |
| | yl)ethyl)-1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 412 | (9S,13S,19aR,22R)-22-benzyl-13-isobutyl-N-(3-methoxyphenethyl)-12-methyl-7,11,14,20-tetraoxo- |
| | 7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 413 | (9S,13S,19aS,22R)-22-benzyl-13-isobutyl-N-(3-methoxyphenethyl)-12-methyl-7,11,14,20-tetraoxo- |
| | 7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 414 | (9S,13S,19aR,22R)-22-benzyl-N-(3-(difluoromethoxy)phenethyl)-13-isobutyl-18-(2-methoxyethyl)-12- |
| | methyl-7,11,14,20-tetraoxo-7,8,9,10,11,12,13,14,17,18,19,19a,20,21,22,23-hexadecahydro-16H- |
| | pyrazino[2′,1′:6,7][1]oxa[4,7,10,14]tetraazacycloheptadecino[17,16-c]quinoline-9-carboxamide |
| 415 | (9S,13S,19aR,22R)-22-benzyl-N-(2-(5,6-dihydro-4H-cyclopenta[d]thiazol-2-yl)ethyl)-13-isobutyl-18-(2- |
| | methoxyethyl)-12-methyl-7,11,14,20-tetraoxo-7,8,9,10,11,12,13,14,17,18,19,19a,20,21,22,23- |
| | hexadecahydro-16H-pyrazino[2′,1′:6,7][1]oxa[4,7,10,14]tetraazacycloheptadecino[17,16-c]quinoline-9- |
| | carboxamide |
| 416 | (4aR,7R,18S,22S)-7-benzyl-3-(2-hydroxyethyl)-22-isobutyl-N-(3-methoxyphenethyl)-21-methyl- |
| | 5,16,20,23-tetraoxo-2,3,4,4a,5,6,7,8,16,17,18,19,20,21,22,23-hexadecahydro-1H-naphtho[1,2- |
| | p]pyrazino[2,1-f][1]oxa[4,7,10,14]tetraazacycloheptadecine-18-carboxamide |
| 417 | (8S,12S,18aR,21R)-21-benzyl-17-cyclopropyl-12-isobutyl-N-(3-methoxyphenethyl)-4,11-dimethyl- |
| | 6,10,13,19-tetraoxo-6,7,8,9,10,11,12,13,16,17,18,18a,19,20,21,22-hexadecahydro-15H- |
| | oxazolo[4′,5′:5,6]benzo[1,2-p]pyrazino[2,1-f][1]oxa[4,7,10,14]tetraazacycloheptadecine-8-carboxamide |
| 418 | (8S,12S,18aR,21R)-21-benzyl-17-cyclopropyl-N-(3-ethoxyphenethyl)-12-isobutyl-4,11-dimethyl- |
| | 6,10,13,19-tetraoxo-6,7,8,9,10,11,12,13,16,17,18,18a,19,20,21,22-hexadecahydro-15H- |
| | oxazolo[4′,5′:5,6]benzo[1,2-p]pyrazino[2,1-f][1]oxa[4,7,10,14]tetraazacycloheptadecine-8-carboxamide |
| 419 | (8S,12S,18aR,21R)-21-benzyl-17-cyclopropyl-N-(3-(difluoromethoxy)phenethyl)-12-isobutyl-4,11- |
| | dimethyl-6,10,13,19-tetraoxo-6,7,8,9,10,11,12,13,16,17,18,18a,19,20,21,22-hexadecahydro-15H- |
| | oxazolo[4′,5′:5,6]benzo[1,2-p]pyrazino[2,1-f][1]oxa[4,7,10,14]tetraazacycloheptadecine-8-carboxamide |
| 420 | (8S,12S,18aR,21R)-21-benzyl-17-cyclopropyl-N-(2-(5,6-dihydro-4H-cyclopenta[d]thiazol-2-yl)ethyl)-12- |
| | isobutyl-4,11-dimethyl-6,10,13,19-tetraoxo-6,7,8,9,10,11,12,13,16,17,18,18a,19,20,21,22- |
| | hexadecahydro-15H-oxazolo[4′,5′:5,6]benzo[1,2-p]pyrazino[2,1- |
| | f][1]oxa[4,7,10,14]tetraazacycloheptadecine-8-carboxamide |
| 421 | (8S,12S,18aR,21R)-21-benzyl-17-cyclopropyl-N-(2-(2,3-dihydrobenzo[b][1,4]dioxin-6-yl)ethyl)-12- |
| | isobutyl-4,11-dimethyl-6,10,13,19-tetraoxo-6,7,8,9,10,11,12,13,16,17,18,18a,19,20,21,22- |
| | hexadecahydro-15H-oxazolo[4′,5′:5,6]benzo[1,2-p]pyrazino[2,1- |
| | f][1]oxa[4,7,10,14]tetraazacycloheptadecine-8-carboxamide |
| 422 | (8R,14S,18S)-8-benzyl-12-(2-cyclohexylethyl)-N-(3-(1,1-difluoroethyl)phenethyl)-14-isobutyl-2,15- |
| | dimethyl-10,13,16,20-tetraoxo-7,8,9,10,11,12,13,14,15,16,17,18,19,20- |
| | tetradecahydrooxazolo[4′,5′:5,6]benzo[1,2-p][1]oxa[4,7,10,14]tetraazacycloheptadecine-18- |
| | carboxamide |
| 423 | (8S,12S,18aR,21R)-21-benzyl-12-isobutyl-N-(3-methoxyphenethyl)-3,11-dimethyl-6,10,13,19-tetraoxo- |
| | 6,7,8,9,10,11,12,13,16,17,18,18a,19,20,21,22-hexadecahydro-3H,15H- |
| | pyrido[2′,1′:6,7][1]oxa[4,7,10,14]tetraazacycloheptadecino[16,17-e]indazole-8-carboxamide |
| 424 | (8S,12S,18aR,21R)-21-benzyl-12-isobutyl-N-(3-methoxyphenethyl)-3,11-dimethyl-6,10,13,19-tetraoxo- |
| | 6,7,8,9,10,11,12,13,15,16,18,18a,19,20,21,22-hexadecahydro-3H- |
| | [1,4]oxazino[3′,4′:6,7][1]oxa[4,7,10,14]tetraazacycloheptadecino[16,17-e]indazole-8-carboxamide |
| 425 | (8S,12S,18aR,21R)-21-benzyl-17-(cyclopropylmethyl)-12-isobutyl-N-(3-methoxyphenethyl)-4,11- |
| | dimethyl-6,10,13,19-tetraoxo-6,7,8,9,10,11,12,13,16,17,18,18a,19,20,21,22-hexadecahydro-15H- |
| | oxazolo[4′,5′:5,6]benzo[1,2-p]pyrazino[2,1-f][1]oxa[4,7,10,14]tetraazacycloheptadecine-8-carboxamide |
| 426 | (8S,12S,18aR,21R)-21-benzyl-17-(cyclopropylmethyl)-N-(3-(difluoromethoxy)phenethyl)-12-isobutyl- |
| | 4,11-dimethyl-6,10,13,19-tetraoxo-6,7,8,9,10,11,12,13,16,17,18,18a,19,20,21,22-hexadecahydro-15H- |
| | oxazolo[4′,5′:5,6]benzo[1,2-p]pyrazino[2,1-f][1]oxa[4,7,10,14]tetraazacycloheptadecine-8-carboxamide |
| 427 | (8S,12S,18aR,21R)-21-benzyl-17-(cyclopropylmethyl)-N-(3-ethoxyphenethyl)-12-isobutyl-4,11-dimethyl- |
| | 6,10,13,19-tetraoxo-6,7,8,9,10,11,12,13,16,17,18,18a,19,20,21,22-hexadecahydro-15H- |
| | oxazolo[4′,5′:5,6]benzo[1,2-p]pyrazino[2,1-f][1]oxa[4,7,10,14]tetraazacycloheptadecine-8-carboxamide |
| 428 | (8S,12S,18aR,21R)-N-(4-(2H-1,2,3-triazol-2-yl)phenethyl)-21-benzyl-17-(cyclopropylmethyl)-12- |
| | isobutyl-4,11-dimethyl-6,10,13,19-tetraoxo-6,7,8,9,10,11,12,13,16,17,18,18a,19,20,21,22- |
| | hexadecahydro-15H-oxazolo[4′,5′:5,6]benzo[1,2-p]pyrazino[2,1- |
| | f][1]oxa[4,7,10,14]tetraazacycloheptadecine-8-carboxamide |
| 429 | (8S,12S,18aR,21R)-21-benzyl-12-isobutyl-3,11-dimethyl-N-(2-(3-methylisoxazol-5-yl)ethyl)-6,10,13,19- |
| | tetraoxo-6,7,8,9,10,11,12,13,16,17,18,18a,19,20,21,22-hexadecahydro-3H,15H- |
| | pyrido[2′,11:6,7][1]oxa[4,7,10,14]tetraazacycloheptadecino[16,17-e]indazole-8-carboxamide |
| 430 | (3R,9S,13S)-N-(2-(benzo[d][1,3]dioxol-5-yl)ethyl)-3-benzyl-9-isobutyl-7,10-dimethyl-5,8,11,15-tetraoxo- |
| | 2,3,4,5,6,7,8,9,10,11,12,13,14,15-tetradecahydropyrido[2′,11:2,3]imidazo[4,5- |
| | p][1]oxa[4,7,10,14]tetraazacycloheptadecine-13-carboxamide |
| 431 | (3S,7S,10R,13R)-13-benzyl-7-isobutyl-N-(3-methoxyphenethyl)-9-(3-methoxypropyl)-6,10-dimethyl- |
| | 1,5,8,11-tetraoxo-1,2,3,4,5,6,7,8,9,10,11,12,13,14-tetradecahydronaphtho[1,2- |
| | p][1]oxa[4,7,10,14]tetraazacycloheptadecine-3-carboxamide |
| 432 | (3′S,7′S,13′R)-13′-benzyl-7′-(cyclopentylmethyl)-6′,9′-dimethyl-N-(2-(3-methylisoxazol-5-yl)ethyl)- |
| | 1′,5′,8′,11′-tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 433 | (3′S,7′S,13′R)-13′-benzyl-7′-isobutyl-N-(3-methoxyphenethyl)-6′,9′-dimethyl-1′,5′,8′,11′-tetraoxo- |
| | 2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 434 | (3′S,7′S,13′R)-13′-benzyl-7′-isobutyl-6′,9′-dimethyl-N-(2-(3-methylisoxazol-5-yl)ethyl)-1′,5′,8′,11′- |
| | tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 435 | (8S,12S,18aR,21R)-21-benzyl-12-(cyclopentylmethyl)-4,11-dimethyl-N-(2-(3-methylisoxazol-5-yl)ethyl)- |
| | 6,10,13,19-tetraoxo-6,7,8,9,10,11,12,13,15,16,18,18a,19,20,21,22-hexadecahydro-[1,4]oxazino[3,4- |
| | floxazolo[4′,5′:5,6]benzo[1,2-p][1]oxa[4,7,10,14]tetraazacycloheptadecine-8-carboxamide |
| 436 | (8S,12S,18aR,21R)-21-benzyl-12-(cyclopentylmethyl)-4,11-dimethyl-N-(2-(3-methylisoxazol-5-yl)ethyl)- |
| | 6,10,13,19-tetraoxo-6,7,8,9,10,11,12,13,16,17,18,18a,19,20,21,22-hexadecahydro-15H- |
| | oxazolo[4′,5′:5,6]benzo[1,2-p]pyrido[2,1-f][1]oxa[4,7,10,14]tetraazacycloheptadecine-8-carboxamide |
| 437 | (8S,12S,18aS,21R)-21-benzyl-12-(cyclopentylmethyl)-4,11-dimethyl-N-(2-(3-methylisoxazol-5-yl)ethyl)- |
| | 6,10,13,19-tetraoxo-6,7,8,9,10,11,12,13,16,17,18,18a,19,20,21,22-hexadecahydro-15H- |
| | oxazolo[4′,5′:5,6]benzo[1,2-p]pyrido[2,1-f][1]oxa[4,7,10,14]tetraazacycloheptadecine-8-carboxamide |
| 438 | (3′R,9′S,13′S)-3′-benzyl-9′-isobutyl-16′-methoxy-N-(3-methoxyphenethyl)-7′,10′-dimethyl-5′,8′,11′,15′- |
| | tetraoxo-2′,3′,4′,5′,8′,9′,10′,11′,12′,13′,14′,15′-dodecahydro-7′H-spiro[cyclopropane-1,6′-pyrido[3,4- |
| | p][1]oxa[4,7,10,14]tetraazacycloheptadecine]-13′-carboxamide |
| 439 | (3′S,7′S,13′R)-13′-benzyl-18′-fluoro-7′-isobutyl-N-(3-methoxyphenethyl)-6′,9′-dimethyl-1′,5′,8′,11′- |
| | tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 440 | (3′S,7′S,13′R)-13′-benzyl-18′-fluoro-7′-isobutyl-6′,9′-dimethyl-N-(2-(3-methylisoxazol-5-yl)ethyl)- |
| | 1′,5′,8′,11′-tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 441 | (3S,7S,13R)-13-benzyl-7-isobutyl-N-(3-methoxyphenethyl)-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[16,17- |
| | f]quinoline-3-carboxamide |
| 442 | (3′R,9′S,13′S)-3′-benzyl-9′-isobutyl-N-(3-methoxyphenethyl)-7′,10′-dimethyl-5′,8′,11′,15′-tetraoxo- |
| | 2′,3′,4′,5′,8′,9′,10′,11′,12′,13′,14′,15′-dodecahydro-7′H-spiro[cyclopropane-1,6′- |
| | pyrido[2′,11:2,3]imidazo[4,5-p][1]oxa[4,7,10,14]tetraazacycloheptadecine]-13′-carboxamide |
| 443 | (9S,13S,19aR,22R)-22-benzyl-3-fluoro-13-isobutyl-12-methyl-N-(2-(3-methylisoxazol-5-yl)ethyl)- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 444 | (7S,11S,17aR,20R)-20-benzyl-11-isobutyl-4,10-dimethyl-N-(2-(3-methylisoxazol-5-yl)ethyl)-5,9,12,18- |
| | tetraoxo-5,6,7,8,9,10,11,12,15,16,17,17a,18,19,20,21-hexadecahydro-14H-dipyrido[2,1-f:3′,4′- |
| | p][1]oxa[4,7,10,14]tetraazacycloheptadecine-7-carboxamide |
| 445 | (3S,7S,13R)-13-benzyl-7-isobutyl-6-methyl-N-(2-(3-methylisoxazol-5-yl)ethyl)-1,5,8,11-tetraoxo-9-(((R)- |
| | tetrahydro-2H-pyran-2-yl)methyl)-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 446 | (3S,7S,13R)-13-benzyl-7-isobutyl-6-methyl-N-(2-(3-methylisoxazol-5-yl)ethyl)-1,5,8,11-tetraoxo-9-(2- |
| | ((S)-tetrahydro-2H-pyran-2-yl)ethyl)-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 447 | (8R,14S,18S)-8-benzyl-14-isobutyl-N-(3-methoxyphenethyl)-12,15-dimethyl-10,13,16,20-tetraoxo- |
| | 2,3,7,8,9,10,13,14,15,16,17,18,19,20-tetradecahydro-12H-spiro[benzofuro[7,6- |
| | p][1]oxa[4,7,10,14]tetraazacycloheptadecine-11,1′-cyclopropane]-18-carboxamide |
| 448 | (3′R,9′S,13′S)-3′-benzyl-9′-isobutyl-7′,10′-dimethyl-N-(2-(3-methylisoxazol-5-yl)ethyl)-5′,8′,11′,15′- |
| | tetraoxo-2′,3′,4′,5′,8′,9′,10′,11′,12′,13′,14′,15′-dodecahydro-7′H-spiro[cyclopropane-1,6′- |
| | pyrido[2′,11:2,3]imidazo[4,5-p][1]oxa[4,7,10,14]tetraazacycloheptadecine]-13′-carboxamide |
| 449 | (3R,9S,13S)-3-benzyl-9-isobutyl-N-(3-methoxyphenethyl)-7,10-dimethyl-5,8,11,15-tetraoxo- |
| | 2,3,4,5,6,7,8,9,10,11,12,13,14,15-tetradecahydropyrido[2′,1′:2,3]imidazo[4,5- |
| | p][1]oxa[4,7,10,14]tetraazacycloheptadecine-13-carboxamide |
| 450 | (3S,7S,13R)-13-benzyl-7-isobutyl-N-(3-methoxyphenethyl)-6,9-dimethyl-1,5,8,11-tetraoxo- |
| | 1,2,3,4,5,6,7,8,9,10,11,12,13,14,19,20-hexadecahydrobenzofuro[4,5- |
| | p][1]oxa[4,7,10,14]tetraazacycloheptadecine-3-carboxamide |
| 451 | (4aR,7R,18S,22S)-7-benzyl-3-(3-hydroxypropyl)-22-isobutyl-N-(3-methoxyphenethyl)-21-methyl- |
| | 5,16,20,23-tetraoxo-2,3,4,4a,5,6,7,8,16,17,18,19,20,21,22,23-hexadecahydro-1H-naphtho[1,2- |
| | p]pyrazino[2,1-f][1]oxa[4,7,10,14]tetraazacycloheptadecine-18-carboxamide |
| 452 | (4aR,7R,18S,22S)-7-benzyl-3-(3-hydroxypropyl)-22-isobutyl-21-methyl-N-(2-(3-methylisoxazol-5- |
| | yl)ethyl)-5,16,20,23-tetraoxo-2,3,4,4a,5,6,7,8,16,17,18,19,20,21,22,23-hexadecahydro-1H-naphtho[1,2- |
| | p]pyrazino[2,1-f][1]oxa[4,7,10,14]tetraazacycloheptadecine-18-carboxamide |
| 453 | (3′S,7′S,13′R)-N-(3-(2H-1,2,3-triazol-2-yl)phenethyl)-13′-benzyl-7′-isobutyl-6′,9′-dimethyl-1′,5′,8′,11′- |
| | tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 454 | (3′S,7′S,13′R)-N-(4-(2H-1,2,3-triazol-2-yl)phenethyl)-13′-benzyl-7′-isobutyl-6′,9′-dimethyl-1′,5′,8′,11′- |
| | tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 455 | (8S,12S,18aR,21R)-21-benzyl-12-isobutyl-11-methyl-N-(2-(3-methylisoxazol-5-yl)ethyl)-6,10,13,19- |
| | tetraoxo-3,4,6,7,8,9,10,11,12,13,16,17,18,18a,19,20,21,22-octadecahydro-15H-benzofuro[7,6- |
| | p]pyrido[2,1-f][1]oxa[4,7,10,14]tetraazacycloheptadecine-8-carboxamide |
| 456 | (8S,12S,18aR,21R)-21-benzyl-12-isobutyl-N-(3-methoxyphenethyl)-11-methyl-6,10,13,19-tetraoxo- |
| | 3,4,6,7,8,9,10,11,12,13,16,17,18,18a,19,20,21,22-octadecahydro-15H-benzofuro[7,6-p]pyrido[2,1- |
| | f][1]oxa[4,7,10,14]tetraazacycloheptadecine-8-carboxamide |
| 457 | (3′S,7′S,13′R)-13′-benzyl-7′-isobutyl-9′-(2-methoxyethyl)-N-(3-methoxyphenethyl)-6′-methyl-1′,5′,8′,11′- |
| | tetraoxo-2′,3′,4′,5′,6′,7′,8′,9′,11′,12′,13′,14′-dodecahydro-1′H-spiro[cyclopropane-1,10′-naphtho[1,2- |
| | p][1]oxa[4,7,10,14]tetraazacycloheptadecine]-3′-carboxamide |
| 458 | 2,2,2-trichloroethyl (9S,13S,19aR,22R)-22-benzyl-13-isobutyl-12-methyl-9-((2-(3-methylisoxazol-5- |
| | yl)ethyl)carbamoyl)-7,11,14,20-tetraoxo-7,8,9,10,11,12,13,14,16,17,19,19a,20,21,22,23- |
| | hexadecahydro-18H-pyrazino[2′,1′:6,7][1]oxa[4,7,10,14]tetraazacycloheptadecino[17,16-c]quinoline-18- |
| | carboxylate |
| 459 | (9S,13S,19aR,22R)-22-benzyl-13-isobutyl-N-(3-methoxyphenethyl)-12,18-dimethyl-7,11,14,20-tetraoxo- |
| | 7,8,9,10,11,12,13,14,17,18,19,19a,20,21,22,23-hexadecahydro-16H- |
| | pyrazino[2′,11:6,7][1]oxa[4,7,10,14]tetraazacycloheptadecino[17,16-c]quinoline-9-carboxamide |
| 460 | (3′S,7′S,13′R)-13′-benzyl-7′-isobutyl-N-(3-methoxyphenethyl)-6′,9′-dimethyl-1′,5′,8′,11′-tetraoxo- |
| | 2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[16,17-f]quinoline]-3′-carboxamide |
| 461 | (9S,13S,19aR,22R)-N-(3-(2H-1,2,3-triazol-2-yl)phenethyl)-22-benzyl-13-isobutyl-12-methyl-7,11,14,20- |
| | tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 462 | (9S,13S,19aS,22R)-N-(3-(2H-1,2,3-triazol-2-yl)phenethyl)-22-benzyl-13-isobutyl-12-methyl-7,11,14,20- |
| | tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 463 | (9S,13S,19aR,22R)-N-(4-(2H-1,2,3-triazol-2-yl)phenethyl)-22-benzyl-13-isobutyl-12-methyl-7,11,14,20- |
| | tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 464 | (9S,13S,19aS,22R)-N-(4-(2H-1,2,3-triazol-2-yl)phenethyl)-22-benzyl-13-isobutyl-12-methyl-7,11,14,20- |
| | tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 465 | (3S,7S,13R)-13-benzyl-7-isobutyl-N-(3-methoxyphenethyl)-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[16,17- |
| | flisoquinoline-3-carboxamide |
| 466 | (7S,11S,17aR,20R)-N-(3-(2H-1,2,3-triazol-2-yl)phenethyl)-20-benzyl-11-isobutyl-4-methoxy-10-methyl- |
| | 5,9,12,18-tetraoxo-5,6,7,8,9,10,11,12,15,16,17,17a,18,19,20,21-hexadecahydro-14H-dipyrido[2,1- |
| | f:3′,4′-p][1]oxa[4,7,10,14]tetraazacycloheptadecine-7-carboxamide |
| 467 | (7S,11S,17aS,20R)-N-(3-(2H-1,2,3-triazol-2-yl)phenethyl)-20-benzyl-11-isobutyl-4-methoxy-10-methyl- |
| | 5,9,12,18-tetraoxo-5,6,7,8,9,10,11,12,15,16,17,17a,18,19,20,21-hexadecahydro-14H-dipyrido[2,1- |
| | f:3′,4′-p][1]oxa[4,7,10,14]tetraazacycloheptadecine-7-carboxamide |
| 468 | (7S,11S,17aR,20R)-N-(4-(2H-1,2,3-triazol-2-yl)phenethyl)-20-benzyl-11-isobutyl-4-methoxy-10-methyl- |
| | 5,9,12,18-tetraoxo-5,6,7,8,9,10,11,12,15,16,17,17a,18,19,20,21-hexadecahydro-14H-dipyrido[2,1- |
| | f:3′,4′-p][1]oxa[4,7,10,14]tetraazacycloheptadecine-7-carboxamide |
| 469 | (7S,11S,22R,24aR)-22-benzyl-7-isobutyl-N-(3-methoxyphenethyl)-8-methyl-6,9,13,24-tetraoxo- |
| | 1,3,4,6,7,8,9,10,11,12,13,21,22,23,24,24a-hexadecahydro-2H-pyrido[2,1-f]pyrido[2′,1′:2,3]imidazo[4,5- |
| | p][1]oxa[4,7,10,14]tetraazacycloheptadecine-11-carboxamide |
| 470 | (7S,11S,22R,24aR)-22-benzyl-7-isobutyl-8-methyl-N-(2-(3-methylisoxazol-5-yl)ethyl)-6,9,13,24- |
| | tetraoxo-1,3,4,6,7,8,9,10,11,12,13,21,22,23,24,24a-hexadecahydro-2H-pyrido[2,1- |
| | f]pyrido[2′,1′:2,3]imidazo[4,5-p][1]oxa[4,7,10,14]tetraazacycloheptadecine-11-carboxamide |
| 471 | (9′R,15′S,19′S)-9′-benzyl-15′-isobutyl-N-(3-methoxyphenethyl)-13′,16′-dimethyl-11′,14′,17′,21′-tetraoxo- |
| | 8′,9′,10′,11′,14′,15′,16′,17′,18′,19′,20′,21′-dodecahydro-13′H-spiro[cyclopropane-1,12′- |
| | [1]oxa[4,7,10,14]tetraazacycloheptadecino[17,16-h]quinoline]-19′-carboxamide |
| 472 | (9S,13S,19aR,22R)-N-(4-(2H-1,2,3-triazol-2-yl)phenethyl)-22-benzyl-13-isobutyl-12-methyl-7,11,14,20- |
| | tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[16,17-f]quinoline-9-carboxamide |
| 473 | (9S,13S,19aS,22R)-N-(4-(2H-1,2,3-triazol-2-yl)phenethyl)-22-benzyl-13-isobutyl-12-methyl-7,11,14,20- |
| | tetraoxo-7,8,9,10,11,12,13,14,17,18,19,19a,20,21,22,23-hexadecahydro-16H- |
| | pyrido[2′,11:6,7][1]oxa[4,7,10,14]tetraazacycloheptadecino[16,17-f]quinoline-9-carboxamide |
| 474 | (9S,13S,19aR,22R)-22-benzyl-13-isobutyl-12-methyl-N-(2-(3-methylisoxazol-5-yl)ethyl)-7,11,14,20- |
| | tetraoxo-7,8,9,10,11,12,13,14,17,18,19,19a,20,21,22,23-hexadecahydro-16H- |
| | pyrido[2′,11:6,7][1]oxa[4,7,10,14]tetraazacycloheptadecino[16,17-f]quinoline-9-carboxamide |
| 475 | (4aR,7R,18S,22S)-7-benzyl-22-isobutyl-21-methyl-N-(2-(3-methylisoxazol-5-yl)ethyl)-5,16,20,23- |
| | tetraoxo-3-(pyridin-3-ylsulfonyl)-2,3,4,4a,5,6,7,8,16,17,18,19,20,21,22,23-hexadecahydro-1H- |
| | naphtho[1,2-p]pyrazino[2,1-f][1]oxa[4,7,10,14]tetraazacycloheptadecine-18-carboxamide |
| 476 | (3′S,7′S,13′R)-13′-benzyl-N-(2-(3-cyclopropylisoxazol-5-yl)ethyl)-7′-isobutyl-6′,9′-dimethyl-1′,5′,8′,11′- |
| | tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 477 | (7S,11S,17aR,20R)-20-benzyl-11-isobutyl-4-methoxy-10-methyl-N-(2-(3-methylisoxazol-5-yl)ethyl)- |
| | 5,9,12,18-tetraoxo-5,6,7,8,9,10,11,12,15,16,17,17a,18,19,20,21-hexadecahydro-14H-dipyrido[2,1- |
| | f:3′,4′-p][1]oxa[4,7,10,14]tetraazacycloheptadecine-7-carboxamide |
| 478 | (7S,11S,17aR,20R)-20-benzyl-N-(2,6-difluorophenethyl)-11-isobutyl-4-methoxy-10-methyl-5,9,12,18- |
| | tetraoxo-5,6,7,8,9,10,11,12,15,16,17,17a,18,19,20,21-hexadecahydro-14H-dipyrido[2,1-f:3′,4′- |
| | p][1]oxa[4,7,10,14]tetraazacycloheptadecine-7-carboxamide |
| 479 | (4aR,7R,18S,22S)-7-benzyl-22-isobutyl-3-((5-methoxypyridin-3-yl)sulfonyl)-21-methyl-N-(2-(3- |
| | methylisoxazol-5-yl)ethyl)-5,16,20,23-tetraoxo-2,3,4,4a,5,6,7,8,16,17,18,19,20,21,22,23- |
| | hexadecahydro-1H-naphtho[1,2-p]pyrazino[2,1-f][1]oxa[4,7,10,14]tetraazacycloheptadecine-18- |
| | carboxamide |
| 480 | (9S,13S,19aR,22R)-22-benzyl-5-fluoro-13-isobutyl-12-methyl-N-(2-(3-methylisoxazol-5-yl)ethyl)- |
| | 7,11,14,20-tetraoxo-7,8,9,10,11,12,13,14,17,18,19,19a,20,21,22,23-hexadecahydro-16H- |
| | pyrido[2′,11:6,7][1]oxa[4,7,10,14]tetraazacycloheptadecino[17,16-c]quinoline-9-carboxamide |
| 481 | (9S,13S,19aR,22R)-N-(4-(2H-1,2,3-triazol-2-yl)phenethyl)-22-benzyl-5-fluoro-13-isobutyl-12-methyl- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 482 | (8S,12S,18aR,21R)-21-benzyl-N-(2,6-difluorophenethyl)-12-isobutyl-4,11-dimethyl-6,10,13,19-tetraoxo- |
| | 6,7,8,9,10,11,12,13,16,17,18,18a,19,20,21,22-hexadecahydro-15H-oxazolo[4′,5′:5,6]benzo[1,2- |
| | p]pyrido[2,1-f][1]oxa[4,7,10,14]tetraazacycloheptadecine-8-carboxamide |
| 483 | (7S,11S,17aR,20R)-20-benzyl-N-(2-(5-cyclopropyl-1,2,4-oxadiazol-3-yl)ethyl)-11-isobutyl-4-methoxy- |
| | 10-methyl-5,9,12,18-tetraoxo-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 484 | (7S,11S,22R,24aR)-22-benzyl-N-(2,6-difluorophenethyl)-7-isobutyl-8-methyl-6,9,13,24-tetraoxo- |
| | 1,3,4,6,7,8,9,10,11,12,13,21,22,23,24,24a-hexadecahydro-2H-pyrido[2,1-f]pyrido[2′,1′:2,3]imidazo[4,5- |
| | p][1]oxa[4,7,10,14]tetraazacycloheptadecine-11-carboxamide |
| 485 | (8S,12S,18aR,21R)-21-benzyl-N-(2,6-difluorophenethyl)-12-isobutyl-5,11-dimethyl-6,10,13,19-tetraoxo- |
| | 6,7,8,9,10,11,12,13,16,17,18,18a,19,20,21,22-hexadecahydro-5H,15H-imidazo[4′,5′:5,6]benzo[1,2- |
| | p]pyrido[2,1-f][1]oxa[4,7,10,14]tetraazacycloheptadecine-8-carboxamide |
| 486 | (9S,13S,19aR,22R)-22-benzyl-N-(2-(5-cyclopropyl-1,2,4-oxadiazol-3-yl)ethyl)-13-isobutyl-12-methyl- |
| | 7,11,14,20-tetraoxo-7,8,9,10,11,12,13,14,17,18,19,19a,20,21,22,23-hexadecahydro-16H- |
| | pyrido[2′,11:6,7][1]oxa[4,7,10,14]tetraazacycloheptadecino[16,17-f]quinoline-9-carboxamide |
| 487 | (9S,13S,19aR,22S)-22-benzyl-13-isobutyl-12-methyl-N-(2-(3-methylisoxazol-5-yl)ethyl)-7,11,14,20- |
| | tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 488 | (3′R,9′S,13′S)-3′-benzyl-9′-isobutyl-17′-methoxy-N-(3-methoxyphenethyl)-7′,10′-dimethyl-5′,8′,11′,15′- |
| | tetraoxo-2′,3′,4′,5′,8′,9′,10′,11′,12′,13′,14′,15′-dodecahydro-7′H-spiro[cyclopropane-1,6′-pyrido[2,3- |
| | p][1]oxa[4,7,10,14]tetraazacycloheptadecine]-13′-carboxamide |
| 489 | (9S,13S,19aR,22R)-22-benzyl-N-(2-(5-cyclopropyl-1,2,4-oxadiazol-3-yl)ethyl)-5-fluoro-13-isobutyl-12- |
| | methyl-7,11,14,20-tetraoxo-7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 490 | (8S,12S,18aR,21R)-21-benzyl-N-(2,6-difluorophenethyl)-12-isobutyl-3,11-dimethyl-6,10,13,19-tetraoxo- |
| | 6,7,8,9,10,11,12,13,16,17,18,18a,19,20,21,22-hexadecahydro-3H,15H- |
| | pyrido[2′,1′:6,7][1]oxa[4,7,10,14]tetraazacycloheptadecino[16,17-e]indazole-8-carboxamide |
| 491 | (8S,12S,18aR,21R)-21-benzyl-N-(2,6-difluorophenethyl)-12-isobutyl-11-methyl-6,10,13,19-tetraoxo- |
| | 3,4,6,7,8,9,10,11,12,13,16,17,18,18a,19,20,21,22-octadecahydro-15H-benzofuro[7,6-p]pyrido[2,1- |
| | f][1]oxa[4,7,10,14]tetraazacycloheptadecine-8-carboxamide |
| 492 | (1r,3S,3′S,7′S,13′R)-N-(4-(2H-1,2,3-triazol-2-yl)phenethyl)-13′-benzyl-7′-isobutyl-3-methoxy-6′,9′- |
| | dimethyl-1′,5′,8′,11′-tetraoxo-2′,3′,4′,5′,6′,7′,8′,9′,11′,12′,13′,14′-dodecahydro-1′H-spiro[cyclobutane-1,10′- |
| | naphtho[1,2-p][1]oxa[4,7,10,14]tetraazacycloheptadecine]-3′-carboxamide |
| 493 | (1r,3S,3′S,7′S,13′R)-N-(4-(2H-1,2,3-triazol-2-yl)phenethyl)-13′-benzyl-7′-isobutyl-3-methoxy-6′,9′- |
| | dimethyl-1′,5′,8′,11′-tetraoxo-2′,3′,4′,5′,6′,7′,8′,9′,11′,12′,13′,14′-dodecahydro-1′H-spiro[cyclobutane-1,10′- |
| | [1]oxa[4,7,10,14]tetraazacycloheptadecino[17,16-c]quinoline]-3′-carboxamide |
| 494 | (3′S,7′S,13′R)-13′-benzyl-7′-isobutyl-N-(3-methoxyphenethyl)-6′,9′-dimethyl-1′,5′,8′,11′-tetraoxo- |
| | 2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[16,17-f]isoquinoline]-3′-carboxamide |
| 495 | (3′S,7′S,13′R)-13′-benzyl-7′-isobutyl-6′,9′-dimethyl-N-(2-(3-methylisoxazol-5-yl)ethyl)-1′,5′,8′,11′- |
| | tetraoxo-1,2′,3,3′,4′,5′,6′,7′,8′,9′,11′,12′,13′,14′-tetradecahydro-1′H-spiro[indene-2,10′- |
| | [1]oxa[4,7,10,14]tetraazacycloheptadecino[17,16-c]quinoline]-3′-carboxamide |
| 496 | (3′S,7′S,13′R)-13′-benzyl-7′-isobutyl-6′,9′-dimethyl-1′,5′,8′,11′-tetraoxo-N-(4-(pyrimidin-2-yl)phenethyl)- |
| | 2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 497 | (9S,13S,19aS,22R)-22-benzyl-N-(2-(5-cyclopropylisoxazol-3-yl)ethyl)-3-fluoro-13-isobutyl-12-methyl- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 498 | (9S,13S,19aR,22R)-22-benzyl-N-(2-(5-cyclopropyl-1,2,4-oxadiazol-3-yl)ethyl)-3-fluoro-13-isobutyl-12- |
| | methyl-7,11,14,20-tetraoxo-7,8,9,10,11,12,13,14,17,18,19,19a,20,21,22,23-hexadecahydro-16H- |
| | pyrido[2′,11:6,7][1]oxa[4,7,10,14]tetraazacycloheptadecino[17,16-c]quinoline-9-carboxamide |
| 499 | (9S,13S,19aR,22R)-22-benzyl-N-(2-(3-cyclopropylisoxazol-5-yl)ethyl)-3-fluoro-13-isobutyl-12-methyl- |
| | 7,11,14,20-tetraoxo-7,8,9,10,11,12,13,14,17,18,19,19a,20,21,22,23-hexadecahydro-16H- |
| | pyrido[2′,11:6,7][1]oxa[4,7,10,14]tetraazacycloheptadecino[17,16-c]quinoline-9-carboxamide |
| 500 | (9S,13S,19aS,22R)-22-benzyl-N-(2-(3-cyclopropylisoxazol-5-yl)ethyl)-3-fluoro-13-isobutyl-12-methyl- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 501 | (9S,13S,19aR,22R)-22-benzyl-N-(2-(3-cyclopropyl-1,2,4-oxadiazol-5-yl)ethyl)-3-fluoro-13-isobutyl-12- |
| | methyl-7,11,14,20-tetraoxo-7,8,9,10,11,12,13,14,17,18,19,19a,20,21,22,23-hexadecahydro-16H- |
| | pyrido[2′,11:6,7][1]oxa[4,7,10,14]tetraazacycloheptadecino[17,16-c]quinoline-9-carboxamide |
| 502 | (3′S,7′S,13′R)-13′-benzyl-7′-isobutyl-6′,9′-dimethyl-1′,5′,8′,11′-tetraoxo-N-(3-(pyrimidin-2-yl)phenethyl)- |
| | 2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 503 | (3S,7S,13R)-13-benzyl-7-isobutyl-N-(3-methoxyphenethyl)-6,9,10,10-tetramethyl-1,5,8,11-tetraoxo- |
| | 1,2,3,4,5,6,7,8,9,10,11,12,13,14-tetradecahydro-[1]oxa[4,7,10,14]tetraazacycloheptadecino[17,16- |
| | c]quinoline-3-carboxamide |
| 504 | (9S,13S,19aR,22R)-22-benzyl-N-(2-(5-cyclopropylisoxazol-3-yl)ethyl)-13-isobutyl-12-methyl-7,11,14,20- |
| | tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[16,17-f]quinoline-9-carboxamide |
| 505 | (9S,13S,19aR,22R)-22-benzyl-N-(2-(3-cyclopropyl-1,2,4-oxadiazol-5-yl)ethyl)-13-isobutyl-12-methyl- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[16,17-f]quinoline-9-carboxamide |
| 506 | (9S,13S,19aR,22R)-22-benzyl-12-ethyl-13-isobutyl-N-(2-(3-methylisoxazol-5-yl)ethyl)-7,11,14,20- |
| | tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 507 | (3S,7S,13R)-13-benzyl-20-fluoro-7-isobutyl-N-(3-methoxyphenethyl)-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[16,17- |
| | f]quinoline-3-carboxamide |
| 508 | (3S,7S,10R,13R)-13-benzyl-10-cyclopropyl-7-isobutyl-6,9-dimethyl-N-(2-(3-methylisoxazol-5-yl)ethyl)- |
| | 1,5,8,11-tetraoxo-1,2,3,4,5,6,7,8,9,10,11,12,13,14-tetradecahydronaphtho[1,2- |
| | p][1]oxa[4,7,10,14]tetraazacycloheptadecine-3-carboxamide |
| 509 | (9S,13S,19aR,22R)-22-benzyl-13-isobutyl-N-(3-methoxyphenethyl)-12-methyl-7,11,14,20-tetraoxo-18- |
| | phenyl-7,8,9,10,11,12,13,14,17,18,19,19a,20,21,22,23-hexadecahydro-16H- |
| | pyrazino[2′,11:6,7][1]oxa[4,7,10,14]tetraazacycloheptadecino[17,16-c]quinoline-9-carboxamide |
| 510 | (9S,13S,19aR,22R)-22-benzyl-13-isobutyl-12-methyl-N-(2-(3-methylisoxazol-5-yl)ethyl)-7,11,14,20- |
| | tetraoxo-18-phenyl-7,8,9,10,11,12,13,14,17,18,19,19a,20,21,22,23-hexadecahydro-16H- |
| | pyrazino[2′,11:6,7][1]oxa[4,7,10,14]tetraazacycloheptadecino[17,16-c]quinoline-9-carboxamide |
| 511 | (7S,11S,17aR,20R)-20-benzyl-N-(2-(3-cyclopropyl-1,2,4-oxadiazol-5-yl)ethyl)-11-isobutyl-4-methoxy- |
| | 10-methyl-5,9,12,18-tetraoxo-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 512 | (7S,11S,17aR,20R)-20-benzyl-N-(2-(5-cyclopropylisoxazol-3-yl)ethyl)-11-isobutyl-4-methoxy-10-methyl- |
| | 5,9,12,18-tetraoxo-5,6,7,8,9,10,11,12,15,16,17,17a,18,19,20,21-hexadecahydro-14H-dipyrido[2,1- |
| | f:3′,4′-p][1]oxa[4,7,10,14]tetraazacycloheptadecine-7-carboxamide |
| 513 | (3R,9S,13S)-3-benzyl-9-isobutyl-16-methoxy-N-(3-methoxyphenethyl)-7,10-dimethyl-5,8,11,15- |
| | tetraoxo-2,3,4,5,6,7,8,9,10,11,12,13,14,15-tetradecahydropyrido[3,2- |
| | p][1]oxa[4,7,10,14]tetraazacycloheptadecine-13-carboxamide |
| 514 | (9S,13S,19aR,22R)-22-benzyl-N-(2-(3-cyclopropylisoxazol-5-yl)ethyl)-13-isobutyl-12-methyl-7,11,14,20- |
| | tetraoxo-7,8,9,10,11,12,13,14,17,18,19,19a,20,21,22,23-hexadecahydro-16H- |
| | pyrido[2′,11:6,7][1]oxa[4,7,10,14]tetraazacycloheptadecino[16,17-f]quinoline-9-carboxamide |
| 515 | (7S,11S,17aRS,20R)-20-benzyl-N-(2-(3-cyclopropyl-1,2,4-oxadiazol-5-yl)ethyl)-11-isobutyl-3-methoxy- |
| | 10-methyl-5,9,12,18-tetraoxo-5,6,7,8,9,10,11,12,15,16,17,17a,18,19,20,21-hexadecahydro-14H- |
| | dipyrido[2,1-f:4′,3′-p][1]oxa[4,7,10,14]tetraazacycloheptadecine-7-carboxamide |
| 516 | (7S,11S,17aRS,20R)-20-benzyl-N-(2-(5-cyclopropyl-1,2,4-oxadiazol-3-yl)ethyl)-11-isobutyl-3-methoxy- |
| | 10-methyl-5,9,12,18-tetraoxo-5,6,7,8,9,10,11,12,15,16,17,17a,18,19,20,21-hexadecahydro-14H- |
| | dipyrido[2,1-f:4′,3′-p][1]oxa[4,7,10,14]tetraazacycloheptadecine-7-carboxamide |
| 517 | (7S,11S,17aR,20R)-20-benzyl-N-(2-(3-cyclopropylisoxazol-5-yl)ethyl)-11-isobutyl-3-methoxy-10-methyl- |
| | 5,9,12,18-tetraoxo-5,6,7,8,9,10,11,12,15,16,17,17a,18,19,20,21-hexadecahydro-14H-dipyrido[2,1- |
| | f:2′,3′-p][1]oxa[4,7,10,14]tetraazacycloheptadecine-7-carboxamide |
| 518 | (7S,11S,17aR,20R)-20-benzyl-N-(2-(5-cyclopropylisoxazol-3-yl)ethyl)-11-isobutyl-3-methoxy-10-methyl- |
| | 5,9,12,18-tetraoxo-5,6,7,8,9,10,11,12,15,16,17,17a,18,19,20,21-hexadecahydro-14H-dipyrido[2,1- |
| | f:2′,3′-p][1]oxa[4,7,10,14]tetraazacycloheptadecine-7-carboxamide |
| 519 | (3′S,7′S,13′R)-13′-benzyl-N-(2-(3-cyclopropylisoxazol-5-yl)ethyl)-20′-fluoro-7′-isobutyl-6′,9′-dimethyl- |
| | 1′,5′,8′,11′-tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 520 | (9S,13S,19aS,22R)-22-benzyl-N-(2-(5-cyclopropyl-1,2,4-oxadiazol-3-yl)ethyl)-5-fluoro-13-isobutyl-12- |
| | methyl-7,11,14,20-tetraoxo-7,8,9,10,11,12,13,14,17,18,19,19a,20,21,22,23-hexadecahydro-16H- |
| | pyrido[2′,11:6,7][1]oxa[4,7,10,14]tetraazacycloheptadecino[17,16-c]quinoline-9-carboxamide |
| 521 | (9S,13S,19aS,22R)-22-benzyl-N-(2-(3-cyclopropyl-1,2,4-oxadiazol-5-yl)ethyl)-5-fluoro-13-isobutyl-12- |
| | methyl-7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 522 | (9S,13S,19aR,22R)-N-(4-(2H-1,2,3-triazol-2-yl)phenethyl)-22-benzyl-3-fluoro-13-isobutyl-12-methyl- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 523 | (7S,11S,17aR,20R)-20-benzyl-N-(2-(3-cyclopropylisoxazol-5-yl)ethyl)-11-isobutyl-4-methoxy-10-methyl- |
| | 5,9,12,18-tetraoxo-5,6,7,8,9,10,11,12,15,16,17,17a,18,19,20,21-hexadecahydro-14H-dipyrido[2,1- |
| | f:3′,4′-p][1]oxa[4,7,10,14]tetraazacycloheptadecine-7-carboxamide |
| 524 | (9S,13S,19aR,22R)-22-benzyl-N-(2-(3-cyclopropylisoxazol-5-yl)ethyl)-5-fluoro-13-isobutyl-12-methyl- |
| | 7,11,14,20-tetraoxo-18-phenyl-7,8,9,10,11,12,13,14,17,18,19,19a,20,21,22,23-hexadecahydro-16H- |
| | pyrazino[2′,11:6,7][1]oxa[4,7,10,14]tetraazacycloheptadecino[17,16-c]quinoline-9-carboxamide |
| 525 | (9S,13S,19aR,22R)-22-benzyl-N-(2-(3-cyclopropyl-1,2,4-oxadiazol-5-yl)ethyl)-5-fluoro-13-isobutyl-12- |
| | methyl-7,11,14,20-tetraoxo-18-phenyl-7,8,9,10,11,12,13,14,17,18,19,19a,20,21,22,23-hexadecahydro- |
| | 16H-pyrazino[2′,11:6,7][1]oxa[4,7,10,14]tetraazacycloheptadecino[17,16-c]quinoline-9-carboxamide |
| 526 | (7S,11S,17aR,20R)-20-benzyl-N-(2-(5-cyclopropylisoxazol-3-yl)ethyl)-11-isobutyl-3-methoxy-10-methyl- |
| | 5,9,12,18-tetraoxo-5,6,7,8,9,10,11,12,15,16,17,17a,18,19,20,21-hexadecahydro-14H-dipyrido[2,1- |
| | f:4′,3′-p][1]oxa[4,7,10,14]tetraazacycloheptadecine-7-carboxamide |
| 527 | (9S,13S,19aR,22R)-22-benzyl-5-chloro-13-isobutyl-12-methyl-N-(2-(3-methylisoxazol-5-yl)ethyl)- |
| | 7,11,14,20-tetraoxo-7,8,9,10,11,12,13,14,17,18,19,19a,20,21,22,23-hexadecahydro-16H- |
| | pyrido[2′,11:6,7][1]oxa[4,7,10,14]tetraazacycloheptadecino[17,16-c]quinoline-9-carboxamide |
| 528 | (9S,13S,19aR,22R)-22-benzyl-5-chloro-13-isobutyl-N-(3-methoxyphenethyl)-12-methyl-7,11,14,20- |
| | tetraoxo-7,8,9,10,11,12,13,14,17,18,19,19a,20,21,22,23-hexadecahydro-16H- |
| | pyrido[2′,11:6,7][1]oxa[4,7,10,14]tetraazacycloheptadecino[17,16-c]quinoline-9-carboxamide |
| 529 | (7S,11S,17aR,20R)-20-benzyl-N-(2-(3-cyclopropyl-1,2,4-oxadiazol-5-yl)ethyl)-11-isobutyl-3-methoxy- |
| | 10-methyl-5,9,12,18-tetraoxo-5,6,7,8,9,10,11,12,15,16,17,17a,18,19,20,21-hexadecahydro-14H- |
| | dipyrido[2,1-f:2′,3′-p][1]oxa[4,7,10,14]tetraazacycloheptadecine-7-carboxamide |
| 530 | (3S,7S,13R)-13-benzyl-7-isobutyl-N-(3-methoxyphenethyl)-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[17,16- |
| | clisoquinoline-3-carboxamide |
| 531 | (8R,14S,18S)-8-benzyl-14-isobutyl-N-(3-methoxyphenethyl)-2,12,15-trimethyl-10,13,16,20-tetraoxo- |
| | 7,8,9,10,11,12,13,14,15,16,17,18,19,20-tetradecahydrobenzofuro[7,6- |
| | p][1]oxa[4,7,10,14]tetraazacycloheptadecine-18-carboxamide |
| 532 | (8R,14S,18S)-8-benzyl-N-(2-(3-cyclopropylisoxazol-5-yl)ethyl)-14-isobutyl-2,12,15-trimethyl- |
| | 10,13,16,20-tetraoxo-7,8,9,10,11,12,13,14,15,16,17,18,19,20-tetradecahydrobenzofuro[7,6- |
| | p][1]oxa[4,7,10,14]tetraazacycloheptadecine-18-carboxamide |
| 533 | (7S,11S,17aR,20R)-20-benzyl-11-isobutyl-4-methoxy-N-(2-(3-methoxyisoxazol-5-yl)ethyl)-2,10- |
| | dimethyl-5,9,12,18-tetraoxo-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 534 | (9S,13S,19aR,22R)-22-benzyl-6-fluoro-13-isobutyl-12-methyl-N-(2-(3-methylisoxazol-5-yl)ethyl)- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 535 | (9S,13S,19aR,22R)-22-benzyl-6-fluoro-13-isobutyl-N-(3-methoxyphenethyl)-12-methyl-7,11,14,20- |
| | tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 536 | (8S,12S,18aR,21R)-21-benzyl-12-isobutyl-N-(3-methoxyphenethyl)-4,11-dimethyl-6,10,13,19-tetraoxo- |
| | 17-phenyl-6,7,8,9,10,11,12,13,16,17,18,18a,19,20,21,22-hexadecahydro-15H- |
| | oxazolo[4′,5′:5,6]benzo[1,2-p]pyrazino[2,1-f][1]oxa[4,7,10,14]tetraazacycloheptadecine-8-carboxamide |
| 537 | (73S,4R,9S,13S)-4-benzyl-16-fluoro-9-isobutyl-N-(3-methoxyphenethyl)-10-methyl-6,8,11,15-tetraoxo- |
| | 2-oxa-5,10,14-triaza-1(3,4)-quinolina-7(3,1)-pyrrolidinacyclopentadecaphane-13-carboxamide |
| 538 | (7S,11S,20R,22aR)-20-benzyl-7-isobutyl-14-methoxy-N-(3-methoxyphenethyl)-8-methyl-6,9,13,22- |
| | tetraoxo-2-phenyl-1,3,4,6,7,8,9,10,11,12,13,19,20,21,22,22a-hexadecahydro-2H-pyrazino[2,1- |
| | f]pyrido[3,4-p][1]oxa[4,7,10,14]tetraazacycloheptadecine-11-carboxamide |
| 539 | (3S,7S,10R,13R)-13-benzyl-N-(2-(5-cyclopropyl-1,2,4-oxadiazol-3-yl)ethyl)-10-((4,4- |
| | difluorocyclohexyl)methyl)-20-fluoro-7-isobutyl-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[17,16- |
| | c]quinoline-3-carboxamide |
| 540 | (3S,7S,10S,13R)-13-benzyl-N-(2-(5-cyclopropyl-1,2,4-oxadiazol-3-yl)ethyl)-10-((4,4- |
| | difluorocyclohexyl)methyl)-20-fluoro-7-isobutyl-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[17,16- |
| | c]quinoline-3-carboxamide |
| 541 | (9S,13S,19aR,22R)-22-benzyl-N-(2-(5-cyclopropyl-1,2,4-oxadiazol-3-yl)ethyl)-5-fluoro-13-isobutyl-12- |
| | methyl-7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[16,17-f]quinoline-9-carboxamide |
| 542 | (7S,11S,20R,22aR)-20-benzyl-7-isobutyl-14-methoxy-8-methyl-N-(2-(3-methylisoxazol-5-yl)ethyl)- |
| | 6,9,13,22-tetraoxo-2-phenyl-1,3,4,6,7,8,9,10,11,12,13,19,20,21,22,22a-hexadecahydro-2H- |
| | pyrazino[2,1-f]pyrido[3,4-p][1]oxa[4,7,10,14]tetraazacycloheptadecine-11-carboxamide |
| 543 | (3′S,7′S,13′R)-13′-benzyl-N-(2-(3-cyclopropylisoxazol-5-yl)ethyl)-7′-isobutyl-6′,9′-dimethyl-1′,5′,8′,11′- |
| | tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[16,17-f]quinoline]-3′-carboxamide |
| 544 | (3′S,7′S,13′R)-13′-benzyl-N-(2-(5-cyclopropyl-1,2,4-oxadiazol-3-yl)ethyl)-7′-isobutyl-6′,9′-dimethyl- |
| | 1′,5′,8′,11′-tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[16,17-f]quinoline]-3′-carboxamide |
| 545 | (3′S,7′S,13′R)-13′-benzyl-N-(2-(3-cyclopropylisoxazol-5-yl)ethyl)-20′-fluoro-7′-isobutyl-6′,9′-dimethyl- |
| | 1′,5′,8′,11′-tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[16,17-f]quinoline]-3′-carboxamide |
| 546 | (3′S,7′S,13′R)-13′-benzyl-N-(2-(5-cyclopropyl-1,2,4-oxadiazol-3-yl)ethyl)-20′-fluoro-7′-isobutyl-6′,9′- |
| | dimethyl-1′,5′,8′,11′-tetraoxo-2′,3′,4′,5′,6′,7′,8′,9′,11′,12′,13′,14′-dodecahydro-1′H-spiro[cyclopropane- |
| | 1,10′-[1]oxa[4,7,10,14]tetraazacycloheptadecino[16,17-f]quinoline]-3′-carboxamide |
| 547 | (3S,7S,10R,13R)-13-benzyl-7-isobutyl-10-isopropyl-6,9-dimethyl-N-(2-(3-methylisoxazol-5-yl)ethyl)- |
| | 1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 548 | (3S,7S,10R,13R)-13-benzyl-7,10-diisobutyl-6,9-dimethyl-N-(2-(3-methylisoxazol-5-yl)ethyl)-1,5,8,11- |
| | tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 549 | (7S,11S,20R,22aS)-20-benzyl-7-isobutyl-14-methoxy-N-(3-methoxyphenethyl)-8-methyl-6,9,13,22- |
| | tetraoxo-2-phenyl-1,3,4,6,7,8,9,10,11,12,13,19,20,21,22,22a-hexadecahydro-2H-pyrazino[2,1- |
| | f]pyrido[3,4-p][1]oxa[4,7,10,14]tetraazacycloheptadecine-11-carboxamide |
| 550 | (7S,11S,20R,22aS)-20-benzyl-7-isobutyl-14-methoxy-8-methyl-N-(2-(3-methylisoxazol-5-yl)ethyl)- |
| | 6,9,13,22-tetraoxo-2-phenyl-1,3,4,6,7,8,9,10,11,12,13,19,20,21,22,22a-hexadecahydro-2H- |
| | pyrazino[2,1-f]pyrido[3,4-p][1]oxa[4,7,10,14]tetraazacycloheptadecine-11-carboxamide |
| 551 | (8R,14S,18S)-8-benzyl-14-isobutyl-N-(3-methoxyphenethyl)-2,12,15-trimethyl-10,13,16,20-tetraoxo- |
| | 7,8,9,10,13,14,15,16,17,18,19,20-dodecahydro-12H-spiro[benzofuro[7,6- |
| | p][1]oxa[4,7,10,14]tetraazacycloheptadecine-11,1′-cyclopropane]-18-carboxamide |
| 552 | (8R,14S,18S)-8-benzyl-N-(2-(3-cyclopropylisoxazol-5-yl)ethyl)-14-isobutyl-2,12,15-trimethyl- |
| | 10,13,16,20-tetraoxo-7,8,9,10,13,14,15,16,17,18,19,20-dodecahydro-12H-spiro[benzofuro[7,6- |
| | p][1]oxa[4,7,10,14]tetraazacycloheptadecine-11,1′-cyclopropane]-18-carboxamide |
| 553 | (8R,14S,18S)-8-benzyl-N-(2-(5-cyclopropyl-1,2,4-oxadiazol-3-yl)ethyl)-14-isobutyl-2,12,15-trimethyl- |
| | 10,13,16,20-tetraoxo-7,8,9,10,13,14,15,16,17,18,19,20-dodecahydro-12H-spiro[benzofuro[7,6- |
| | p][1]oxa[4,7,10,14]tetraazacycloheptadecine-11,1′-cyclopropane]-18-carboxamide |
| 554 | (8R,14S,18S)-8-benzyl-N-(2-(3-cyclopropyl-1,2,4-oxadiazol-5-yl)ethyl)-14-isobutyl-2,12,15-trimethyl- |
| | 10,13,16,20-tetraoxo-7,8,9,10,13,14,15,16,17,18,19,20-dodecahydro-12H-spiro[benzofuro[7,6- |
| | p][1]oxa[4,7,10,14]tetraazacycloheptadecine-11,1′-cyclopropane]-18-carboxamide |
| 555 | (3′S,7′S,13′R)-13′-benzyl-7′-isobutyl-N-(3-methoxyphenethyl)-6′,9′-dimethyl-1′,5′,8′,11′-tetraoxo- |
| | 2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]isoquinoline]-3′-carboxamide |
| 556 | (3′S,7′S,13′R)-13′-benzyl-N-(2-(3-cyclopropylisoxazol-5-yl)ethyl)-7′-isobutyl-6′,9′-dimethyl-1′,5′,8′,11′- |
| | tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]isoquinoline]-3′-carboxamide |
| 557 | (3R,6S,9S,13S)-3-benzyl-N-(2-(5-cyclopropyl-1,2,4-oxadiazol-3-yl)ethyl)-6-((4,4- |
| | difluorocyclohexyl)methyl)-9-isobutyl-16-methoxy-7,10-dimethyl-5,8,11,15-tetraoxo- |
| | 2,3,4,5,6,7,8,9,10,11,12,13,14,15-tetradecahydropyrido[3,4- |
| | p][1]oxa[4,7,10,14]tetraazacycloheptadecine-13-carboxamide |
| 558 | (3R,6R,9S,13S)-3-benzyl-N-(2-(5-cyclopropyl-1,2,4-oxadiazol-3-yl)ethyl)-6-((4,4- |
| | difluorocyclohexyl)methyl)-9-isobutyl-16-methoxy-7,10-dimethyl-5,8,11,15-tetraoxo- |
| | 2,3,4,5,6,7,8,9,10,11,12,13,14,15-tetradecahydropyrido[3,4- |
| | p][1]oxa[4,7,10,14]tetraazacycloheptadecine-13-carboxamide |
| 559 | (9S,13S,19aR,22R)-22-benzyl-13-isobutyl-12-methyl-7,11,14,20-tetraoxo-N-(2-(3- |
| | (trifluoromethyl)isoxazol-5-yl)ethyl)-7,8,9,10,11,12,13,14,17,18,19,19a,20,21,22,23-hexadecahydro- |
| | 16H-pyrido[2′,1′:6,7][1]oxa[4,7,10,14]tetraazacycloheptadecino[17,16-c]quinoline-9-carboxamide |
| 560 | (9S,13S,19aR,22R)-22-benzyl-5-fluoro-13-isobutyl-12-methyl-7,11,14,20-tetraoxo-N-(2-(3- |
| | (trifluoromethyl)isoxazol-5-yl)ethyl)-7,8,9,10,11,12,13,14,17,18,19,19a,20,21,22,23-hexadecahydro- |
| | 16H-pyrido[2′,1′:6,7][1]oxa[4,7,10,14]tetraazacycloheptadecino[17,16-c]quinoline-9-carboxamide |
| 561 | (8R,14S,18S)-8-benzyl-N-(2-(5-cyclopropyl-1,2,4-oxadiazol-3-yl)ethyl)-14-isobutyl-2,12,15-trimethyl- |
| | 10,13,16,20-tetraoxo-7,8,9,10,11,12,13,14,15,16,17,18,19,20-tetradecahydrobenzofuro[7,6- |
| | p][1]oxa[4,7,10,14]tetraazacycloheptadecine-18-carboxamide |
| 562 | (8R,14S,18S)-8-benzyl-N-(2-(3-cyclopropyl-1,2,4-oxadiazol-5-yl)ethyl)-14-isobutyl-2,12,15-trimethyl- |
| | 10,13,16,20-tetraoxo-7,8,9,10,11,12,13,14,15,16,17,18,19,20-tetradecahydrobenzofuro[7,6- |
| | p][1]oxa[4,7,10,14]tetraazacycloheptadecine-18-carboxamide |
| 563 | (9S,13S,19aR,22R)-22-benzyl-N-(2-(5-cyclopropyl-1,2,4-oxadiazol-3-yl)ethyl)-6-fluoro-13-isobutyl-12- |
| | methyl-7,11,14,20-tetraoxo-7,8,9,10,11,12,13,14,17,18,19,19a,20,21,22,23-hexadecahydro-16H- |
| | pyrido[2′,11:6,7][1]oxa[4,7,10,14]tetraazacycloheptadecino[17,16-c]quinoline-9-carboxamide |
| 564 | (3S,7S,10R,13R)-13-benzyl-10-((benzyloxy)methyl)-7-isobutyl-6,9-dimethyl-N-(2-(3-methylisoxazol-5- |
| | yl)ethyl)-1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 565 | (7S,11S,17aR,20R)-20-benzyl-N-(2-(5-cyclopropylisoxazol-3-yl)ethyl)-11-isobutyl-4-methoxy-2,10- |
| | dimethyl-5,9,12,18-tetraoxo-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 566 | (3S,7S,10R,13R)-13-benzyl-7-isobutyl-10-(2-methoxyethyl)-6,9-dimethyl-N-(2-(3-methylisoxazol-5- |
| | yl)ethyl)-1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 567 | (7S,11S,17aR,20R)-20-benzyl-N-(2-(5-cyclopropyl-1,2,4-oxadiazol-3-yl)ethyl)-11-isobutyl-4-methoxy- |
| | 2,10-dimethyl-5,9,12,18-tetraoxo-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 568 | (7S,11S,17aR,20R)-20-benzyl-11-isobutyl-4-methoxy-2,10-dimethyl-5,9,12,18-tetraoxo-N-(2-(3- |
| | (trifluoromethyl)isoxazol-5-yl)ethyl)-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 569 | (7S,11S,19bRS,22R)-22-benzyl-11-isobutyl-4-methoxy-N-(3-methoxyphenethyl)-10-methyl-5,9,12,20- |
| | tetraoxo-5,6,7,8,9,10,11,12,15,19b,20,21,22,23-tetradecahydro-14H- |
| | pyrido[3′,4′:16,17][1]oxa[4,7,10,14]tetraazacycloheptadecino[6,7-a]isoquinoline-7-carboxamide |
| 570 | (7S,11S,20R,22aR)-20-benzyl-2,7-diisobutyl-14-methoxy-8-methyl-N-(2-(3-methylisoxazol-5-yl)ethyl)- |
| | 6,9,13,22-tetraoxo-1,3,4,6,7,8,9,10,11,12,13,19,20,21,22,22a-hexadecahydro-2H-pyrazino[2,1- |
| | f]pyrido[3,4-p][1]oxa[4,7,10,14]tetraazacycloheptadecine-11-carboxamide |
| 571 | (3R,6R,9S,13S)-3-benzyl-N-(2-(5-cyclopropyl-1,2,4-oxadiazol-3-yl)ethyl)-9-isobutyl-16-methoxy-7,10- |
| | dimethyl-5,8,11,15-tetraoxo-6-(2-(3-phenyl-1,2,4-oxadiazol-5-yl)ethyl)- |
| | 2,3,4,5,6,7,8,9,10,11,12,13,14,15-tetradecahydropyrido[3,4- |
| | p][1]oxa[4,7,10,14]tetraazacycloheptadecine-13-carboxamide |
| 572 | (3S,7S,10S,13R)-13-benzyl-7-isobutyl-N-(3-methoxyphenethyl)-6,9,10-trimethyl-1,5,8,11-tetraoxo- |
| | 1,2,3,4,5,6,7,8,9,10,11,12,13,14-tetradecahydro-[1]oxa[4,7,10,14]tetraazacycloheptadecino[17,16- |
| | c]quinoline-3-carboxamide |
| 573 | (3S,7S,10R,13R)-13-benzyl-7-isobutyl-6,9,10-trimethyl-N-(2-(3-methylisoxazol-5-yl)ethyl)-1,5,8,11- |
| | tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 574 | (3S,7S,10R,13R)-13-benzyl-N-(2-(3-cyclopropylisoxazol-5-yl)ethyl)-7-isobutyl-6,9,10-trimethyl-1,5,8,11- |
| | tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 575 | (8R,11RS,14S,18S)-8-benzyl-14-isobutyl-N-(3-methoxyphenethyl)-2,11,12,15-tetramethyl-10,13,16,20- |
| | tetraoxo-7,8,9,10,11,12,13,14,15,16,17,18,19,20-tetradecahydrobenzofuro[7,6- |
| | p][1]oxa[4,7,10,14]tetraazacycloheptadecine-18-carboxamide |
| 576 | (8R,11RS,14S,18S)-8-benzyl-14-isobutyl-2,11,12,15-tetramethyl-N-(2-(3-methylisoxazol-5-yl)ethyl)- |
| | 10,13,16,20-tetraoxo-7,8,9,10,11,12,13,14,15,16,17,18,19,20-tetradecahydrobenzofuro[7,6- |
| | p][1]oxa[4,7,10,14]tetraazacycloheptadecine-18-carboxamide |
| 577 | (3R,6S,9S,13S)-3-benzyl-N-(2-(5-cyclopropyl-1,2,4-oxadiazol-3-yl)ethyl)-9-isobutyl-16-methoxy-7,10- |
| | dimethyl-5,8,11,15-tetraoxo-6-(2-(3-phenyl-1,2,4-oxadiazol-5-yl)ethyl)- |
| | 2,3,4,5,6,7,8,9,10,11,12,13,14,15-tetradecahydropyrido[3,4- |
| | p][1]oxa[4,7,10,14]tetraazacycloheptadecine-13-carboxamide |
| 578 | (3S,7S,10R,13R)-13-benzyl-10-(2-(benzyloxy)ethyl)-7-isobutyl-6,9-dimethyl-N-(2-(3-methylisoxazol-5- |
| | yl)ethyl)-1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 579 | (3S,7S,10S,13R)-13-benzyl-10-(2-(benzyloxy)ethyl)-7-isobutyl-6,9-dimethyl-N-(2-(3-methylisoxazol-5- |
| | yl)ethyl)-1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 580 | (7′S,11′S,17a′R,20′R)-20′-benzyl-11′-isobutyl-4′-methoxy-N-(2-(3-methoxyisoxazol-5-yl)ethyl)-2′,10′- |
| | dimethyl-5′,9′,12′,18′-tetraoxo-5′,6′,7′,8′,9′,10′,11′,12′,14′,15′,17′,17a′,18′,19′,20′,21′- |
| | hexadecahydrospiro[cyclopropane-1,16′-dipyrido[2,1-f:3′,4′- |
| | p][1]oxa[4,7,10,14]tetraazacycloheptadecine]-7′-carboxamide |
| 581 | (7S,11S,17aR,20R)-20-benzyl-11-isobutyl-4-(methoxy-d3)-N-(2-(3-methoxyisoxazol-5-yl)ethyl)-2,10- |
| | dimethyl-5,9,12,18-tetraoxo-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 582 | (3R,9S,13S)-3-benzyl-7-(2-cyclohexylethyl)-9-isobutyl-16-methoxy-N-(2-(3-methoxyisoxazol-5-yl)ethyl)- |
| | 10-methyl-5,8,11,15-tetraoxo-2,3,4,5,6,7,8,9,10,11,12,13,14,15-tetradecahydropyrido[3,4- |
| | p][1]oxa[4,7,10,14]tetraazacycloheptadecine-13-carboxamide |
| 583 | (3R,9S,13S)-N-(2-(benzo[d][1,3]dioxol-5-yl)ethyl)-3-benzyl-9-isobutyl-7,10,18-trimethyl-5,8,11,15- |
| | tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 584 | (13S,4R,10S,14S)-4-benzyl-14-isobutyl-74-methoxy-N-(3-methoxyphenethyl)-13-methyl-2,8,12,15- |
| | tetraoxo-6-oxa-3,9,13-triaza-1(3,1)-pyrrolidina-7(1,2)-benzenacyclopentadecaphane-10-carboxamide |
| 585 | (3R,9S,13S)-N-(2-(benzo[d][1,3]dioxol-5-yl)ethyl)-3-benzyl-7-(2-cyclohexylethyl)-9-isobutyl-17,18- |
| | dimethoxy-10-methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 586 | (3R,9S,13S)-3-benzyl-7-(2-cyclohexylethyl)-9-isobutyl-17,18-dimethoxy-N-(3-methoxyphenethyl)-10- |
| | methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 587 | (7S,11S,20R,22aR)-20-benzyl-7-isobutyl-15-methoxy-N-(3-methoxyphenethyl)-8-methyl-6,9,13,22- |
| | tetraoxo-1,3,4,6,7,8,9,10,11,12,13,19,20,21,22,22a-hexadecahydro-2H-benzo[p]pyrido[2,1- |
| | f][1]oxa[4,7,10,14]tetraazacycloheptadecine-11-carboxamide |
| 588 | (7S,11S,20R,22aR)-20-benzyl-7-isobutyl-15-methoxy-N-(3-methoxyphenethyl)-8-methyl-6,9,13,22- |
| | tetraoxo-1,3,4,6,7,8,9,10,11,12,13,19,20,21,22,22a-hexadecahydrobenzo[p][1,4]oxazino[3,4- |
| | f][1]oxa[4,7,10,14]tetraazacycloheptadecine-11-carboxamide |
| 589 | (3R,9S,13S)-N-(2-(benzo[d][1,3]dioxol-5-yl)ethyl)-3-benzyl-7-(2-cyclohexylethyl)-9-isobutyl-10-methyl- |
| | 5,8,11,15-tetraoxo-2,3,4,5,6,7,8,9,10,11,12,13,14,15-tetradecahydropyrido[3,4- |
| | p][1]oxa[4,7,10,14]tetraazacycloheptadecine-13-carboxamide |
| 590 | (3R,9S,13S)-3-benzyl-7-(2-cyclohexylethyl)-9-isobutyl-N-(3-methoxyphenethyl)-10-methyl-5,8,11,15- |
| | tetraoxo-2,3,4,5,6,7,8,9,10,11,12,13,14,15-tetradecahydropyrido[3,4- |
| | p][1]oxa[4,7,10,14]tetraazacycloheptadecine-13-carboxamide |
| 591 | (7S,11S,20R,22aR)-20-benzyl-7-isobutyl-14-methoxy-N-(3-methoxyphenethyl)-8-methyl-6,9,13,22- |
| | tetraoxo-1,3,4,6,7,8,9,10,11,12,13,19,20,21,22,22a-hexadecahydro-2H-benzo[p]pyrido[2,1- |
| | f][1]oxa[4,7,10,14]tetraazacycloheptadecine-11-carboxamide |
| 592 | (7S,11S,17aS,20R)-20-benzyl-N-(2-(2,3-dihydrobenzofuran-6-yl)ethyl)-11-isobutyl-10-methyl-5,9,12,18- |
| | tetraoxo-5,6,7,8,9,10,11,12,15,16,17,17a,18,19,20,21-hexadecahydro-14H-dipyrido[2,1-f:3′,4′- |
| | p][1]oxa[4,7,10,14]tetraazacycloheptadecine-7-carboxamide |
| 593 | (7S,11S,17aR,20R)-20-benzyl-N-(2-(2,3-dihydrobenzofuran-6-yl)ethyl)-11-isobutyl-10-methyl-5,9,12,18- |
| | tetraoxo-5,6,7,8,9,10,11,12,15,16,17,17a,18,19,20,21-hexadecahydro-14H-dipyrido[2,1-f:3′,4′- |
| | p][1]oxa[4,7,10,14]tetraazacycloheptadecine-7-carboxamide |
| 594 | (7S,11S,17aR,20R)-20-benzyl-N-(2-(2,3-dihydrobenzo[b][1,4]dioxin-6-yl)ethyl)-11-isobutyl-10-methyl- |
| | 5,9,12,18-tetraoxo-5,6,7,8,9,10,11,12,15,16,17,17a,18,19,20,21-hexadecahydro-14H-dipyrido[2,1- |
| | f:3′,4′-p][1]oxa[4,7,10,14]tetraazacycloheptadecine-7-carboxamide |
| 595 | (3S,7S,13R)-13-benzyl-7-isobutyl-N-(3-methoxyphenethyl)-6,9-dimethyl-1,5,8,11-tetraoxo- |
| | 1,2,3,4,5,6,7,8,9,10,11,12,13,14-tetradecahydronaphtho[1,2- |
| | p][1]oxa[4,7,10,14]tetraazacycloheptadecine-3-carboxamide |
| 596 | (3R,9S,13S)-3-benzyl-N-(2-(chroman-7-yl)ethyl)-7-(2-cyclohexylethyl)-9-isobutyl-17,18-dimethoxy-10- |
| | methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 597 | (3R,9S,13S)-3-benzyl-7-(2-cyclohexylethyl)-N-(2-(2,3-dihydrobenzofuran-6-yl)ethyl)-9-isobutyl-17,18- |
| | dimethoxy-10-methyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 598 | (8R,14S,18S)-8-benzyl-14-isobutyl-N-(3-methoxyphenethyl)-2,12,15-trimethyl-10,13,16,20-tetraoxo- |
| | 7,8,9,10,11,12,13,14,15,16,17,18,19,20-tetradecahydrooxazolo[4′,5′:5,6]benzo[1,2- |
| | p][1]oxa[4,7,10,14]tetraazacycloheptadecine-18-carboxamide |
| 599 | (3S,7S,13R)-13-benzyl-N-(2-(chroman-7-yl)ethyl)-9-(2-cyclohexylethyl)-7-isobutyl-6-methyl-1,5,8,11- |
| | tetraoxo-1,2,3,4,5,6,7,8,9,10,11,12,13,14-tetradecahydronaphtho[1,2- |
| | p][1]oxa[4,7,10,14]tetraazacycloheptadecine-3-carboxamide |
| 600 | (3S,7S,13R)-13-benzyl-9-(2-cyclohexylethyl)-N-(2-(2,3-dihydrobenzofuran-6-yl)ethyl)-7-isobutyl-6- |
| | methyl-1,5,8,11-tetraoxo-1,2,3,4,5,6,7,8,9,10,11,12,13,14-tetradecahydronaphtho[1,2- |
| | p][1]oxa[4,7,10,14]tetraazacycloheptadecine-3-carboxamide |
| 601 | (7S,11S,20R,22aS)-20-benzyl-7-isobutyl-14,16-dimethoxy-N-(3-methoxyphenethyl)-8-methyl-6,9,13,22- |
| | tetraoxo-1,3,4,6,7,8,9,10,11,12,13,19,20,21,22,22a-hexadecahydro-2H-benzo[p]pyrido[2,1- |
| | f][1]oxa[4,7,10,14]tetraazacycloheptadecine-11-carboxamide |
| 602 | (7S,11S,20R,22aR)-20-benzyl-7-isobutyl-14,16-dimethoxy-N-(3-methoxyphenethyl)-8-methyl-6,9,13,22- |
| | tetraoxo-1,3,4,6,7,8,9,10,11,12,13,19,20,21,22,22a-hexadecahydro-2H-benzo[p]pyrido[2,1- |
| | f][1]oxa[4,7,10,14]tetraazacycloheptadecine-11-carboxamide |
| 603 | (4aR,7R,18S,22S)-7-benzyl-22-isobutyl-N-(3-methoxyphenethyl)-21-methyl-5,16,20,23-tetraoxo- |
| | 2,3,4,4a,5,6,7,8,16,17,18,19,20,21,22,23-hexadecahydro-1H-naphtho[1,2-p]pyrido[2,1- |
| | f][1]oxa[4,7,10,14]tetraazacycloheptadecine-18-carboxamide |
| 604 | (4aS,7R,18S,22S)-7-benzyl-22-isobutyl-N-(3-methoxyphenethyl)-21-methyl-5,16,20,23-tetraoxo- |
| | 2,3,4,4a,5,6,7,8,16,17,18,19,20,21,22,23-hexadecahydro-1H-naphtho[1,2-p]pyrido[2,1- |
| | f][1]oxa[4,7,10,14]tetraazacycloheptadecine-18-carboxamide |
| 605 | (7S,11S,20R,22aR)-20-benzyl-2-ethyl-7-isobutyl-14-methoxy-N-(3-methoxyphenethyl)-8-methyl- |
| | 6,9,13,22-tetraoxo-1,3,4,6,7,8,9,10,11,12,13,19,20,21,22,22a-hexadecahydro-2H-benzo[p]pyrazino[2,1- |
| | f][1]oxa[4,7,10,14]tetraazacycloheptadecine-11-carboxamide |
| 606 | (4aS,7R,18S,22S)-7-benzyl-22-isobutyl-21-methyl-N-(2-(3-methylisoxazol-5-yl)ethyl)-5,16,20,23- |
| | tetraoxo-2,3,4,4a,5,6,7,8,16,17,18,19,20,21,22,23-hexadecahydro-1H-naphtho[1,2-p]pyrido[2,1- |
| | f][1]oxa[4,7,10,14]tetraazacycloheptadecine-18-carboxamide |
| 607 | (4aR,7R,18S,22S)-7-benzyl-22-isobutyl-21-methyl-N-(2-(3-methylisoxazol-5-yl)ethyl)-5,16,20,23- |
| | tetraoxo-2,3,4,4a,5,6,7,8,16,17,18,19,20,21,22,23-hexadecahydro-1H-naphtho[1,2-p]pyrido[2,1- |
| | f][1]oxa[4,7,10,14]tetraazacycloheptadecine-18-carboxamide |
| 608 | (4aR,7R,18S,22S)-7-benzyl-22-isobutyl-21-methyl-N-(2-(3-methylisoxazol-5-yl)ethyl)-5,16,20,23- |
| | tetraoxo-1,2,4,4a,5,6,7,8,16,17,18,19,20,21,22,23-hexadecahydronaphtho[1,2-p][1,4]oxazino[3,4- |
| | f][1]oxa[4,7,10,14]tetraazacycloheptadecine-18-carboxamide |
| 609 | (7S,11S,20R,22aR)-20-benzyl-7-isobutyl-14,16-dimethoxy-8-methyl-N-(2-(3-methylisoxazol-5-yl)ethyl)- |
| | 6,9,13,22-tetraoxo-1,3,4,6,7,8,9,10,11,12,13,19,20,21,22,22a-hexadecahydro-2H-benzo[p]pyrido[2,1- |
| | f][1]oxa[4,7,10,14]tetraazacycloheptadecine-11-carboxamide |
| 610 | (3S,7S,13R)-N-(2-(benzo[d][1,3]dioxol-5-yl)ethyl)-13-benzyl-9-(2-cyclohexylethyl)-7-isobutyl-6-methyl- |
| | 1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 611 | (7S,11S,20R,22aR)-20-benzyl-7-isobutyl-2-isopropyl-14-methoxy-N-(3-methoxyphenethyl)-8-methyl- |
| | 6,9,13,22-tetraoxo-1,3,4,6,7,8,9,10,11,12,13,19,20,21,22,22a-hexadecahydro-2H-benzo[p]pyrazino[2,1- |
| | f][1]oxa[4,7,10,14]tetraazacycloheptadecine-11-carboxamide |
| 612 | (7S,11S,20R,22aR)-20-benzyl-2,7-diisobutyl-14-methoxy-N-(3-methoxyphenethyl)-8-methyl-6,9,13,22- |
| | tetraoxo-1,3,4,6,7,8,9,10,11,12,13,19,20,21,22,22a-hexadecahydro-2H-benzo[p]pyrazino[2,1- |
| | f][1]oxa[4,7,10,14]tetraazacycloheptadecine-11-carboxamide |
| 613 | (4aR,7R,18S,22S)-7-benzyl-N-(3,5-dimethoxyphenethyl)-3,22-diisobutyl-21-methyl-5,16,20,23-tetraoxo- |
| | 2,3,4,4a,5,6,7,8,16,17,18,19,20,21,22,23-hexadecahydro-1H-naphtho[1,2-p]pyrazino[2,1- |
| | f][1]oxa[4,7,10,14]tetraazacycloheptadecine-18-carboxamide |
| 614 | (8S,12S,18aR,21R)-21-benzyl-12-isobutyl-N-(3-methoxyphenethyl)-4,11-dimethyl-6,10,13,19-tetraoxo- |
| | 6,7,8,9,10,11,12,13,16,17,18,18a,19,20,21,22-hexadecahydro-15H-oxazolo[4′,5′:5,6]benzo[1,2- |
| | p]pyrido[2,1-f][1]oxa[4,7,10,14]tetraazacycloheptadecine-8-carboxamide |
| 615 | (8S,12S,18aS,21R)-21-benzyl-12-isobutyl-N-(3-methoxyphenethyl)-4,11-dimethyl-6,10,13,19-tetraoxo- |
| | 6,7,8,9,10,11,12,13,16,17,18,18a,19,20,21,22-hexadecahydro-15H-oxazolo[4′,5′:5,6]benzo[1,2- |
| | p]pyrido[2,1-f][1]oxa[4,7,10,14]tetraazacycloheptadecine-8-carboxamide |
| 616 | (3R,9S,13S)-N-(2-(benzo[d][1,3]dioxol-5-yl)ethyl)-3-benzyl-16-(difluoromethoxy)-9-isobutyl-7,10- |
| | dimethyl-5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 617 | (4aR,7R,18S,22S)-7-benzyl-3,22-diisobutyl-21-methyl-N-(2-(3-methylisoxazol-5-yl)ethyl)-5,16,20,23- |
| | tetraoxo-2,3,4,4a,5,6,7,8,16,17,18,19,20,21,22,23-hexadecahydro-1H-naphtho[1,2-p]pyrazino[2,1- |
| | f][1]oxa[4,7,10,14]tetraazacycloheptadecine-18-carboxamide |
| 618 | (4aR,7R,18S,22S)-7-benzyl-N-(3,5-dimethoxyphenethyl)-22-isobutyl-3-isopropyl-21-methyl-5,16,20,23- |
| | tetraoxo-2,3,4,4a,5,6,7,8,16,17,18,19,20,21,22,23-hexadecahydro-1H-naphtho[1,2-p]pyrazino[2,1- |
| | f][1]oxa[4,7,10,14]tetraazacycloheptadecine-18-carboxamide |
| 619 | (8S,12S,18aR,21R)-21-benzyl-12,17-diisobutyl-N-(3-methoxyphenethyl)-4,11-dimethyl-6,10,13,19- |
| | tetraoxo-6,7,8,9,10,11,12,13,16,17,18,18a,19,20,21,22-hexadecahydro-15H- |
| | oxazolo[4′,5′:5,6]benzo[1,2-p]pyrazino[2,1-f][1]oxa[4,7,10,14]tetraazacycloheptadecine-8-carboxamide |
| 620 | (8S,12S,18aR,21R)-21-benzyl-12,17-diisobutyl-4,11-dimethyl-N-(2-(3-methylisoxazol-5-yl)ethyl)- |
| | 6,10,13,19-tetraoxo-6,7,8,9,10,11,12,13,16,17,18,18a,19,20,21,22-hexadecahydro-15H- |
| | oxazolo[4′,5′:5,6]benzo[1,2-p]pyrazino[2,1-f][1]oxa[4,7,10,14]tetraazacycloheptadecine-8-carboxamide |
| 621 | (8S,12S,18aS,21R)-21-benzyl-12,17-diisobutyl-4,11-dimethyl-N-(2-(3-methylisoxazol-5-yl)ethyl)- |
| | 6,10,13,19-tetraoxo-6,7,8,9,10,11,12,13,16,17,18,18a,19,20,21,22-hexadecahydro-15H- |
| | oxazolo[4′,5′:5,6]benzo[1,2-p]pyrazino[2,1-f][1]oxa[4,7,10,14]tetraazacycloheptadecine-8-carboxamide |
| 622 | (9S,13S,19aR,22R)-22-benzyl-13,18-diisobutyl-12-methyl-N-(2-(3-methylisoxazol-5-yl)ethyl)- |
| | 7,11,14,20-tetraoxo-7,8,9,10,11,12,13,14,17,18,19,19a,20,21,22,23-hexadecahydro-16H- |
| | pyrazino[2′,1′:6,7][1]oxa[4,7,10,14]tetraazacycloheptadecino[17,16-c]quinoline-9-carboxamide |
| 623 | (4aR,7R,18S,22S)-7-benzyl-22-isobutyl-3-isopropyl-21-methyl-N-(2-(3-methylisoxazol-5-yl)ethyl)- |
| | 5,16,20,23-tetraoxo-2,3,4,4a,5,6,7,8,16,17,18,19,20,21,22,23-hexadecahydro-1H-naphtho[1,2- |
| | p]pyrazino[2,1-f][1]oxa[4,7,10,14]tetraazacycloheptadecine-18-carboxamide |
| 624 | (4aRS,7R,17S,21S)-7-benzyl-21-isobutyl-N-(3-methoxyphenethyl)-12,20-dimethyl-5,15,19,22-tetraoxo- |
| | 1,2,4,4a,5,6,7,8,15,16,17,18,19,20,21,22-hexadecahydroisoxazolo[4′,5′:5,6]benzo[1,2- |
| | p][1,4]oxazino[3,4-f][1]oxa[4,7,10,14]tetraazacycloheptadecine-17-carboxamide |
| 625 | (8S,12S,18aR,21R)-21-benzyl-N-(3-(difluoromethoxy)phenethyl)-12-isobutyl-4,11-dimethyl-6,10,13,19- |
| | tetraoxo-6,7,8,9,10,11,12,13,15,16,18,18a,19,20,21,22-hexadecahydro-[1,4]oxazino[3,4- |
| | floxazolo[4′,5′:5,6]benzo[1,2-p][1]oxa[4,7,10,14]tetraazacycloheptadecine-8-carboxamide |
| 626 | (3S,7S,13R)-N-(2-(benzo[d][1,3]dioxol-5-yl)ethyl)-13-benzyl-7-isobutyl-6-methyl-1,5,8,11-tetraoxo-9-(3- |
| | phenylpropyl)-1,2,3,4,5,6,7,8,9,10,11,12,13,14-tetradecahydronaphtho[1,2- |
| | p][1]oxa[4,7,10,14]tetraazacycloheptadecine-3-carboxamide |
| 627 | (9S,13S,19aR,22R)-22-benzyl-N-(2-(2,3-dihydrobenzo[b][1,4]dioxin-6-yl)ethyl)-3-fluoro-13-isobutyl-12- |
| | methyl-7,11,14,20-tetraoxo-7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 628 | (8′R,14′S,18′S)-8′-benzyl-14′-isobutyl-N-(3-methoxyphenethyl)-2′,12′,15′-trimethyl-10′,13′,16′,20′- |
| | tetraoxo-7′,8′,9′,10′,13′,14′,15′,16′,17′,18′,19′,20′-dodecahydro-12′H-spiro[cyclopropane-1,11′- |
| | oxazolo[4′,5′:5,6]benzo[1,2-p][1]oxa[4,7,10,14]tetraazacycloheptadecine]-18′-carboxamide |
| 629 | (8S,12S,18aR,21R)-21-benzyl-12-isobutyl-3,11-dimethyl-N-(2-(3-methylisoxazol-5-yl)ethyl)-6,10,13,19- |
| | tetraoxo-6,7,8,9,10,11,12,13,16,17,18,18a,19,20,21,22-hexadecahydro-15H- |
| | isoxazolo[4′,5′:5,6]benzo[1,2-p]pyrido[2,1-f][1]oxa[4,7,10,14]tetraazacycloheptadecine-8-carboxamide |
| 630 | (8S,12S,18aR,21R)-21-benzyl-12-(cyclopentylmethyl)-3,11-dimethyl-N-(2-(3-methylisoxazol-5-yl)ethyl)- |
| | 6,10,13,19-tetraoxo-6,7,8,9,10,11,12,13,16,17,18,18a,19,20,21,22-hexadecahydro-15H- |
| | isoxazolo[4′,5′:5,6]benzo[1,2-p]pyrido[2,1-f][1]oxa[4,7,10,14]tetraazacycloheptadecine-8-carboxamide |
| 631 | (8S,12S,18aS,21R)-21-benzyl-12-(cyclopentylmethyl)-3,11-dimethyl-N-(2-(3-methylisoxazol-5-yl)ethyl)- |
| | 6,10,13,19-tetraoxo-6,7,8,9,10,11,12,13,16,17,18,18a,19,20,21,22-hexadecahydro-15H- |
| | isoxazolo[4′,5′:5,6]benzo[1,2-p]pyrido[2,1-f][1]oxa[4,7,10,14]tetraazacycloheptadecine-8-carboxamide |
| 632 | (8S,12S,18aR,21R)-21-benzyl-12-isobutyl-4,11-dimethyl-N-(2-(3-methylisoxazol-5-yl)ethyl)-6,10,13,19- |
| | tetraoxo-6,7,8,9,10,11,12,13,16,17,18,18a,19,20,21,22-hexadecahydro-15H- |
| | oxazolo[4′,5′:5,6]benzo[1,2-p]pyrido[2,1-f][1]oxa[4,7,10,14]tetraazacycloheptadecine-8-carboxamide |
| 633 | (1r,3S,3′S,7′S,13′R)-13′-benzyl-7′-isobutyl-3-methoxy-6′,9′-dimethyl-N-(2-(3-methylisoxazol-5-yl)ethyl)- |
| | 1′,5′,8′,11′-tetraoxo-2′,3′,4′,5′,6′,7′,8′,9′,11′,12′,13′,14′-dodecahydro-1′H-spiro[cyclobutane-1,10′- |
| | naphtho[1,2-p][1]oxa[4,7,10,14]tetraazacycloheptadecine]-3′-carboxamide |
| 634 | (8S,12S,18aR,21R)-21-benzyl-N-(2,6-difluorophenethyl)-12-isobutyl-3,11-dimethyl-6,10,13,19-tetraoxo- |
| | 6,7,8,9,10,11,12,13,16,17,18,18a,19,20,21,22-hexadecahydro-15H-isoxazolo[4′,5′:5,6]benzo[1,2- |
| | p]pyrido[2,1-f][1]oxa[4,7,10,14]tetraazacycloheptadecine-8-carboxamide |
| 635 | (8S,12S,18aS,21R)-21-benzyl-N-(2,6-difluorophenethyl)-12-isobutyl-3,11-dimethyl-6,10,13,19-tetraoxo- |
| | 6,7,8,9,10,11,12,13,16,17,18,18a,19,20,21,22-hexadecahydro-15H-isoxazolo[4′,5′:5,6]benzo[1,2- |
| | p]pyrido[2,1-f][1]oxa[4,7,10,14]tetraazacycloheptadecine-8-carboxamide |
| 636 | (9S,13S,19aR,22R)-22-benzyl-N-(2,6-difluorophenethyl)-5-fluoro-13-isobutyl-12-methyl-7,11,14,20- |
| | tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 637 | (9S,13S,19aR,22R)-22-benzyl-N-(2-(5-cyclopropyl-1,2,4-oxadiazol-3-yl)ethyl)-5-fluoro-13-isobutyl-12- |
| | methyl-7,11,14,20-tetraoxo-7,8,9,10,11,12,13,14,17,18,19,19a,20,21,22,23-hexadecahydro-16H- |
| | pyrido[2′,11:6,7][1]oxa[4,7,10,14]tetraazacycloheptadecino[17,16-c]quinoline-9-carboxamide |
| 638 | (9S,13S,19aR,22R)-22-benzyl-N-(2-(5-cyclopropylisoxazol-3-yl)ethyl)-3-fluoro-13-isobutyl-12-methyl- |
| | 7,11,14,20-tetraoxo-7,8,9,10,11,12,13,14,17,18,19,19a,20,21,22,23-hexadecahydro-16H- |
| | pyrido[2′,11:6,7][1]oxa[4,7,10,14]tetraazacycloheptadecino[17,16-c]quinoline-9-carboxamide |
| 639 | (9S,13S,19aR,22R)-22-benzyl-N-(2-(5-cyclopropylisoxazol-3-yl)ethyl)-5-fluoro-13-isobutyl-12-methyl- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 640 | (9S,13S,19aR,22R)-22-benzyl-N-(2-(3-cyclopropylisoxazol-5-yl)ethyl)-5-fluoro-13-isobutyl-12-methyl- |
| | 7,11,14,20-tetraoxo-7,8,9,10,11,12,13,14,17,18,19,19a,20,21,22,23-hexadecahydro-16H- |
| | pyrido[2′,11:6,7][1]oxa[4,7,10,14]tetraazacycloheptadecino[17,16-c]quinoline-9-carboxamide |
| 641 | (9S,13S,19aR,22R)-22-benzyl-N-(2-(3-cyclopropyl-1,2,4-oxadiazol-5-yl)ethyl)-5-fluoro-13-isobutyl-12- |
| | methyl-7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 642 | (9S,13S,19aR,22R)-22-benzyl-N-(2-(3-cyclopropylisoxazol-5-yl)ethyl)-13-isobutyl-12-methyl-7,11,14,20- |
| | tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 643 | (9S,13S,19aR,22R)-22-benzyl-N-(2-(5-cyclopropylisoxazol-3-yl)ethyl)-5-fluoro-13-isobutyl-12-methyl- |
| | 7,11,14,20-tetraoxo-7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 644 | (9S,13S,19aR,22R)-22-benzyl-N-(2-(3-cyclopropylisoxazol-5-yl)ethyl)-5-fluoro-13-isobutyl-12-methyl- |
| | 7,11,14,20-tetraoxo-7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 645 | (9S,13S,19aR,22R)-22-benzyl-N-(2-(3-cyclopropyl-1,2,4-oxadiazol-5-yl)ethyl)-5-fluoro-13-isobutyl-12- |
| | methyl-7,11,14,20-tetraoxo-7,8,9,10,11,12,13,14,16,17,19,19a,20,21,22,23-hexadecahydro- |
| | [1,4]oxazino[3′,4′:6,7][1]oxa[4,7,10,14]tetraazacycloheptadecino[17,16-c]quinoline-9-carboxamide |
| 646 | (9S,13S,19aR,22R)-22-benzyl-N-(2-(3-cyclopropyl-1,2,4-oxadiazol-5-yl)ethyl)-12-ethyl-13-isobutyl- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 647 | (3R,9S,13S)-3-benzyl-9-isobutyl-17-methoxy-N-(3-methoxyphenethyl)-7,10-dimethyl-5,8,11,15- |
| | tetraoxo-2,3,4,5,6,7,8,9,10,11,12,13,14,15-tetradecahydropyrido[4,3- |
| | p][1]oxa[4,7,10,14]tetraazacycloheptadecine-13-carboxamide |
| 648 | (3′R,9′S,13′S)-3′-benzyl-N-(2-(3-cyclopropylisoxazol-5-yl)ethyl)-9′-isobutyl-17′-methoxy-7′,10′-dimethyl- |
| | 5′,8′,11′,15′-tetraoxo-2′,3′,4′,5′,8′,9′,10′,11′,12′,13′,14′,15′-dodecahydro-7′H-spiro[cyclopropane-1,6′- |
| | pyrido[4,3-p][1]oxa[4,7,10,14]tetraazacycloheptadecine]-13′-carboxamide |
| 649 | (3′R,9′S,13′S)-3′-benzyl-N-(2-(5-cyclopropyl-1,2,4-oxadiazol-3-yl)ethyl)-9′-isobutyl-17′-methoxy-7′,10′- |
| | dimethyl-5′,8′,11′,15′-tetraoxo-2′,3′,4′,5′,8′,9′,10′,11′,12′,13′,14′,15′-dodecahydro-7′H-spiro[cyclopropane- |
| | 1,6′-pyrido[4,3-p][1]oxa[4,7,10,14]tetraazacycloheptadecine]-13′-carboxamide |
| 650 | (3′R,9′S,13′S)-3′-benzyl-9′-isobutyl-17′-methoxy-N-(3-methoxyphenethyl)-7′,10′-dimethyl-5′,8′,11′,15′- |
| | tetraoxo-2′,3′,4′,5′,8′,9′,10′,11′,12′,13′,14′,15′-dodecahydro-7′H-spiro[cyclopropane-1,6′-pyrido[4,3- |
| | p][1]oxa[4,7,10,14]tetraazacycloheptadecine]-13′-carboxamide |
| 651 | (7S,11S,20R,22aR)-N-(4-(2H-1,2,3-triazol-2-yl)phenethyl)-20-benzyl-14-fluoro-7-isobutyl-8-methyl- |
| | 6,9,13,22-tetraoxo-1,3,4,6,7,8,9,10,11,12,13,19,20,21,22,22a-hexadecahydro-2H-benzo[p]pyrido[2,1- |
| | f][1]oxa[4,7,10,14]tetraazacycloheptadecine-11-carboxamide |
| 652 | (7S,11S,17aRS,20R)-20-benzyl-N-(2-(3-cyclopropylisoxazol-5-yl)ethyl)-11-isobutyl-3-methoxy-10- |
| | methyl-5,9,12,18-tetraoxo-5,6,7,8,9,10,11,12,15,16,17,17a,18,19,20,21-hexadecahydro-14H- |
| | dipyrido[2,1-f:4′,3′-p][1]oxa[4,7,10,14]tetraazacycloheptadecine-7-carboxamide |
| 653 | (8S,12S,18aR,21R)-N-(4-(2H-1,2,3-triazol-2-yl)phenethyl)-21-benzyl-12-isobutyl-11-methyl-6,10,13,19- |
| | tetraoxo-6,7,8,9,10,11,12,13,16,17,18,18a,19,20,21,22-hexadecahydro-15H- |
| | [1,3]dioxolo[4′,5′:5,6]benzo[1,2-p]pyrido[2,1-f][1]oxa[4,7,10,14]tetraazacycloheptadecine-4,4-d2-8- |
| | carboxamide |
| 654 | (3′S,7′S,13′R)-13′-benzyl-7′-isobutyl-6′,9′-dimethyl-N-(2-(3-methylisoxazol-5-yl)ethyl)-1′,5′,8′,11′- |
| | tetraoxo-2′,3′,4′,5′,6′,7′,8′,9′,11′,12′,13′,14′-dodecahydro-1′H-spiro[cyclopropane-1,10′-naphtho[1,2- |
| | p][1]oxa[4,7,10,14]tetraazacycloheptadecine]-3′-carboxamide |
| 655 | (9S,13S,19aR,22R)-22-benzyl-N-(2-(3-cyclopropylisoxazol-5-yl)ethyl)-5-fluoro-13-isobutyl-12-methyl- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[16,17-f]quinoline-9-carboxamide |
| 656 | (7S,11S,17aR,20R)-20-benzyl-N-(2-(3-cyclopropyl-1,2,4-oxadiazol-5-yl)ethyl)-11-isobutyl-4-methoxy- |
| | 2,10-dimethyl-5,9,12,18-tetraoxo-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 657 | (7S,11S,17aR,20R)-20-benzyl-N-(2-(3-cyclopropyl-1,2,4-oxadiazol-5-yl)ethyl)-11-isobutyl-4-(methoxy- |
| | d3)-2,10-dimethyl-5,9,12,18-tetraoxo-5,6,7,8,9,10,11,12,15,16,17,17a,18,19,20,21-hexadecahydro- |
| | 14H-dipyrido[2,1-f:3′,4′-p][1]oxa[4,7,10,14]tetraazacycloheptadecine-7-carboxamide |
| 658 | (7S,11S,17aR,20R)-20-benzyl-N-(2-(3-cyclopropylisoxazol-5-yl)ethyl)-11-isobutyl-4-methoxy-2,10- |
| | dimethyl-5,9,12,18-tetraoxo-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 659 | (7S,11S,17aR,20R)-20-benzyl-N-(2-(3-cyclopropylisoxazol-5-yl)ethyl)-11-isobutyl-4-(methoxy-d3)-2,10- |
| | dimethyl-5,9,12,18-tetraoxo-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 660 | (9S,13S,19aR,22R)-22-benzyl-N-(2-(3-cyclopropylisoxazol-5-yl)ethyl)-6-fluoro-13-isobutyl-12-methyl- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 661 | (3R,6S,9S,13S)-3-benzyl-N-(2-(3-cyclopropylisoxazol-5-yl)ethyl)-6-((4,4-difluorocyclohexyl)methyl)-9- |
| | isobutyl-16-methoxy-7,10-dimethyl-5,8,11,15-tetraoxo-2,3,4,5,6,7,8,9,10,11,12,13,14,15- |
| | tetradecahydropyrido[3,4-p][1]oxa[4,7,10,14]tetraazacycloheptadecine-13-carboxamide |
| 662 | (3R,6R,9S,13S)-3-benzyl-N-(2-(3-cyclopropylisoxazol-5-yl)ethyl)-6-((4,4-difluorocyclohexyl)methyl)-9- |
| | isobutyl-16-methoxy-7,10-dimethyl-5,8,11,15-tetraoxo-2,3,4,5,6,7,8,9,10,11,12,13,14,15- |
| | tetradecahydropyrido[3,4-p][1]oxa[4,7,10,14]tetraazacycloheptadecine-13-carboxamide |
| 663 | (7S,11S,17aR,20R)-20-benzyl-N-(2-(3-cyclopropylisoxazol-5-yl)ethyl)-16,16-difluoro-11-isobutyl-4- |
| | methoxy-10-methyl-5,9,12,18-tetraoxo-5,6,7,8,9,10,11,12,15,16,17,17a,18,19,20,21-hexadecahydro- |
| | 14H-dipyrido[2,1-f:3′,4′-p][1]oxa[4,7,10,14]tetraazacycloheptadecine-7-carboxamide |
| 664 | (7S,11S,17aR,20R)-20-benzyl-N-(2-(5-cyclopropylisoxazol-3-yl)ethyl)-11-isobutyl-4-(methoxy-d3)-2,10- |
| | dimethyl-5,9,12,18-tetraoxo-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 665 | (9S,13S,19aR,22R)-22-benzyl-N-(2-(2-cyclopropyl-2H-1,2,3-triazol-4-yl)ethyl)-5-fluoro-13-isobutyl-12- |
| | methyl-7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 666 | (7S,11S,17aR,20R)-20-benzyl-N-(2-(5-cyclopropyl-1,2,4-oxadiazol-3-yl)ethyl)-11-isobutyl-4-(methoxy- |
| | d3)-2,10-dimethyl-5,9,12,18-tetraoxo-5,6,7,8,9,10,11,12,15,16,17,17a,18,19,20,21-hexadecahydro- |
| | 14H-dipyrido[2,1-f:3′,4′-p][1]oxa[4,7,10,14]tetraazacycloheptadecine-7-carboxamide |
| 667 | (7S,11S,17aS,20R)-20-benzyl-11-isobutyl-4-methoxy-2,10-dimethyl-5,9,12,18-tetraoxo-N-(2-(3- |
| | (trifluoromethyl)isoxazol-5-yl)ethyl)-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 668 | (7′S,11′S,17a′R,20′R)-20′-benzyl-N-(2-(3-cyclopropylisoxazol-5-yl)ethyl)-11′-isobutyl-4′-methoxy-10′- |
| | methyl-5′,9′,12′,18′-tetraoxo-5′,6′,7′,8′,9′,10′,11′,12′,14′,15′,17′,17a′,18′,19′,20′,21′- |
| | hexadecahydrospiro[cyclopropane-1,16′-dipyrido[2,1-f:3′,4′- |
| | p][1]oxa[4,7,10,14]tetraazacycloheptadecine]-7′-carboxamide |
| 669 | (7′S,11′S,17a′S,20′R)-20′-benzyl-N-(2-(3-cyclopropylisoxazol-5-yl)ethyl)-11′-isobutyl-4′-methoxy-10′- |
| | methyl-5′,9′,12′,18′-tetraoxo-5′,6′,7′,8′,9′,10′,11′,12′,14′,15′,17′,17a′,18′,19′,20′,21′- |
| | hexadecahydrospiro[cyclopropane-1,16′-dipyrido[2,1-f:3′,4′- |
| | p][1]oxa[4,7,10,14]tetraazacycloheptadecine]-7′-carboxamide |
| 670 | (7S,11S,17aS,20R)-20-benzyl-N-(2-(3-cyclopropylisoxazol-5-yl)ethyl)-11-isobutyl-4-methoxy-10-methyl- |
| | 5,9,12,18-tetraoxo-16-phenyl-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 671 | (7S,11S,17aS,20R)-20-benzyl-N-(2-(3-cyclopropylisoxazol-5-yl)ethyl)-11-isobutyl-4-methoxy-10-methyl- |
| | 5,9,12,18-tetraoxo-16-phenyl-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 672 | (7S,11S,17aR,20R)-20-benzyl-N-(2-(3-cyclopropylisoxazol-5-yl)ethyl)-11-isobutyl-4-methoxy-10-methyl- |
| | 5,9,12,18-tetraoxo-16-phenyl-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 673 | (7S,11S,17aR,20R)-20-benzyl-N-(2-(3-cyclopropylisoxazol-5-yl)ethyl)-11-isobutyl-4-methoxy-10-methyl- |
| | 5,9,12,18-tetraoxo-16-phenyl-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 674 | (9S,13S,19aS,22R)-22-benzyl-N-(2-(3-cyclopropylisoxazol-5-yl)ethyl)-13-isobutyl-12-methyl-7,11,14,20- |
| | tetraoxo-18-phenyl-7,8,9,10,11,12,13,14,17,18,19,19a,20,21,22,23-hexadecahydro-16H- |
| | pyrido[2′,11:6,7][1]oxa[4,7,10,14]tetraazacycloheptadecino[17,16-c]quinoline-9-carboxamide |
| 675 | (9S,13S,19aS,22R)-22-benzyl-N-(2-(3-cyclopropylisoxazol-5-yl)ethyl)-13-isobutyl-12-methyl-7,11,14,20- |
| | tetraoxo-18-phenyl-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 676 | (9S,13S,19aR,22R)-22-benzyl-N-(2-(3-cyclopropylisoxazol-5-yl)ethyl)-13-isobutyl-12-methyl-7,11,14,20- |
| | tetraoxo-18-phenyl-7,8,9,10,11,12,13,14,17,18,19,19a,20,21,22,23-hexadecahydro-16H- |
| | pyrido[2′,11:6,7][1]oxa[4,7,10,14]tetraazacycloheptadecino[17,16-c]quinoline-9-carboxamide |
| 677 | (9S,13S,19aR,22R)-22-benzyl-N-(2-(3-cyclopropylisoxazol-5-yl)ethyl)-13-isobutyl-12-methyl-7,11,14,20- |
| | tetraoxo-18-phenyl-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 678 | (7S,11S,19aR,22R)-22-benzyl-11-isobutyl-4-methoxy-10-methyl-N-(2-(3-methylisoxazol-5-yl)ethyl)- |
| | 5,9,12,20-tetraoxo-5,6,7,8,9,10,11,12,19,19a,20,21,22,23-tetradecahydro-14H- |
| | pyrido[3′,4′:16,17][1]oxa[4,7,10,14]tetraazacycloheptadecino[7,6-b]isoquinoline-7-carboxamide |
| 679 | (7S,11S,19bR,22R)-22-benzyl-11-isobutyl-4-methoxy-10-methyl-N-(2-(3-methylisoxazol-5-yl)ethyl)- |
| | 5,9,12,20-tetraoxo-5,6,7,8,9,10,11,12,15,19b,20,21,22,23-tetradecahydro-14H- |
| | pyrido[3′,4′:16,17][1]oxa[4,7,10,14]tetraazacycloheptadecino[6,7-a]isoquinoline-7-carboxamide |
| 680 | (3S,7S,10R,13R)-13-benzyl-7-isobutyl-N-(3-methoxyphenethyl)-6,9,10-trimethyl-1,5,8,11-tetraoxo- |
| | 1,2,3,4,5,6,7,8,9,10,11,12,13,14-tetradecahydro-[1]oxa[4,7,10,14]tetraazacycloheptadecino[17,16- |
| | c]quinoline-3-carboxamide |
| 681 | (3R,6R,9S,13S)-3-benzyl-N-(2-(5-cyclopropyl-1,2,4-oxadiazol-3-yl)ethyl)-9-isobutyl-16-methoxy-7,10- |
| | dimethyl-5,8,11,15-tetraoxo-6-phenyl-2,3,4,5,6,7,8,9,10,11,12,13,14,15-tetradecahydropyrido[3,4- |
| | p][1]oxa[4,7,10,14]tetraazacycloheptadecine-13-carboxamide |
| 682 | (3R,6R,9S,13S)-3-benzyl-N-(2-(5-cyclopropylisoxazol-3-yl)ethyl)-6-((4,4-difluorocyclohexyl)methyl)-9- |
| | isobutyl-16-methoxy-7,10-dimethyl-5,8,11,15-tetraoxo-2,3,4,5,6,7,8,9,10,11,12,13,14,15- |
| | tetradecahydropyrido[3,4-p][1]oxa[4,7,10,14]tetraazacycloheptadecine-13-carboxamide |
| 683 | (7′S,11′S,17a′R,20′R)-20′-benzyl-11′-isobutyl-4′-methoxy-2′,10′-dimethyl-N-(2-(3-methylisoxazol-5- |
| | yl)ethyl)-5′,9′,12′,18′-tetraoxo-5′,6′,7′,8′,9′,10′,11′,12′,14′,15′,17′,17a′,18′,19′,20′,21′- |
| | hexadecahydrospiro[cyclopropane-1,16′-dipyrido[2,1-f:3′,4′- |
| | p][1]oxa[4,7,10,14]tetraazacycloheptadecine]-7′-carboxamide |
| 684 | (7′S,11′S,17a′S,20′R)-20′-benzyl-11′-isobutyl-4′-methoxy-N-(2-(3-methoxyisoxazol-5-yl)ethyl)-2′,10′- |
| | dimethyl-5′,9′,12′,18′-tetraoxo-5′,6′,7′,8′,9′,10′,11′,12′,14′,15′,17′,17a′,18′,19′,20′,21′- |
| | hexadecahydrospiro[cyclopropane-1,16′-dipyrido[2,1-f:3′,4′- |
| | p][1]oxa[4,7,10,14]tetraazacycloheptadecine]-7′-carboxamide |
| 685 | (7S,11S,17aR,20R)-20-benzyl-N-(2-(2-cyclopropyl-2H-1,2,3-triazol-4-yl)ethyl)-11-isobutyl-4-(methoxy- |
| | d3)-2,10-dimethyl-5,9,12,18-tetraoxo-5,6,7,8,9,10,11,12,15,16,17,17a,18,19,20,21-hexadecahydro- |
| | 14H-dipyrido[2,1-f:3′,4′-p][1]oxa[4,7,10,14]tetraazacycloheptadecine-7-carboxamide |
| 686 | (3′S,7′S,13′R)-13′-benzyl-7′-isobutyl-N-(2-(3-methoxyisoxazol-5-yl)ethyl)-6′,9′-dimethyl-1′,5′,8′,11′- |
| | tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 687 | (3R,9S,13S)-N-(2-(benzo[d][1,3]dioxol-5-yl)ethyl)-3-benzyl-9-isobutyl-7,10-dimethyl-5,8,11,15-tetraoxo- |
| | 2,3,4,5,6,7,8,9,10,11,12,13,14,15-tetradecahydrobenzo[p][1]oxa[4,7,10,14]tetraazacycloheptadecine- |
| | 13-carboxamide |
| 688 | (3R,9S,13S)-3-benzyl-N-(2-(2,3-dihydrobenzo[b][1,4]dioxin-6-yl)ethyl)-9-isobutyl-7,10-dimethyl- |
| | 5,8,11,15-tetraoxo-2,3,4,5,6,7,8,9,10,11,12,13,14,15- |
| | tetradecahydrobenzo[p][1]oxa[4,7,10,14]tetraazacycloheptadecine-13-carboxamide |
| 689 | (3′S,7′S,13′R)-13′-benzyl-N-(2-(3-cyclopropylisoxazol-5-yl)ethyl)-20′-fluoro-7′-isobutyl-6′,9′-dimethyl- |
| | 1′,5′,8′,11′-tetraoxo-2′,3′,4′,5′,6′,7′,8′,9′,11′,12′,13′,14′-dodecahydro-1′H-spiro[cyclobutane-1,10′- |
| | [1]oxa[4,7,10,14]tetraazacycloheptadecino[17,16-c]quinoline]-3′-carboxamide |
| 690 | (3′S,7′S,13′R)-13′-benzyl-20′-fluoro-7′-isobutyl-6′,9′-dimethyl-N-(2-(3-methylisoxazol-5-yl)ethyl)- |
| | 1′,5′,8′,11′-tetraoxo-2′,3′,4′,5′,6′,7′,8′,9′,11′,12′,13′,14′-dodecahydro-1′H-spiro[cyclobutane-1,10′- |
| | [1]oxa[4,7,10,14]tetraazacycloheptadecino[17,16-c]quinoline]-3′-carboxamide |
| 691 | (3′S,7′S,13′R)-13′-benzyl-N-(2-(5-cyclopropyl-1,2,4-oxadiazol-3-yl)ethyl)-20′-fluoro-7′-isobutyl-6′,9′- |
| | dimethyl-1′,5′,8′,11′-tetraoxo-2′,3′,4′,5′,6′,7′,8′,9′,11′,12′,13′,14′-dodecahydro-1′H-spiro[cyclobutane-1,10′- |
| | [1]oxa[4,7,10,14]tetraazacycloheptadecino[17,16-c]quinoline]-3′-carboxamide |
| 692 | (3′S,7′S,13′R)-13′-benzyl-N-(2-(3-cyclopropyl-1,2,4-oxadiazol-5-yl)ethyl)-20′-fluoro-7′-isobutyl-6′,9′- |
| | dimethyl-1′,5′,8′,11′-tetraoxo-2′,3′,4′,5′,6′,7′,8′,9′,11′,12′,13′,14′-dodecahydro-1′H-spiro[cyclobutane-1,10′- |
| | [1]oxa[4,7,10,14]tetraazacycloheptadecino[17,16-c]quinoline]-3′-carboxamide |
| 693 | (3S,7S,10R,13R)-13-benzyl-10-(cyclopentylmethyl)-N-(2-(5-cyclopropyl-1,2,4-oxadiazol-3-yl)ethyl)-20- |
| | fluoro-7-isobutyl-6,9-dimethyl-1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 694 | (3S,7S,10S,13R)-13-benzyl-10-(cyclopentylmethyl)-N-(2-(5-cyclopropyl-1,2,4-oxadiazol-3-yl)ethyl)-20- |
| | fluoro-7-isobutyl-6,9-dimethyl-1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 695 | (3S,7S,10S,13R)-13-benzyl-7-isobutyl-6,9-dimethyl-N-(2-(3-methylisoxazol-5-yl)ethyl)-10-(naphthalen- |
| | 1-ylmethyl)-1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 696 | (3S,7S,10R,13R)-13-benzyl-10-(cyclohexylmethyl)-N-(2-(3-cyclopropyl-1,2,4-oxadiazol-5-yl)ethyl)-20- |
| | fluoro-7-isobutyl-6,9-dimethyl-1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 697 | (3S,7S,10R,13R)-13-benzyl-10-(cyclohexylmethyl)-N-(2-(3-cyclopropylisoxazol-5-yl)ethyl)-20-fluoro-7- |
| | isobutyl-6,9-dimethyl-1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 698 | (3S,7S,10R,13R)-13-benzyl-7-isobutyl-6,9-dimethyl-N-(2-(3-methylisoxazol-5-yl)ethyl)-10-(naphthalen- |
| | 1-ylmethyl)-1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 699 | (3S,7S,10R,13R)-13-benzyl-10-(2,2-difluoroethyl)-7-isobutyl-6,9-dimethyl-N-(2-(3-methylisoxazol-5- |
| | yl)ethyl)-1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 700 | (3S,7S,13R)-N-(2-(benzo[d][1,3]dioxol-5-yl)ethyl)-13-benzyl-9-(((S)-chroman-3-yl)methyl)-7-isobutyl-6- |
| | methyl-1,5,8,11-tetraoxo-1,2,3,4,5,6,7,8,9,10,11,12,13,14-tetradecahydronaphtho[1,2- |
| | p][1]oxa[4,7,10,14]tetraazacycloheptadecine-3-carboxamide |
| 701 | (3S,7S,13R)-9-(2-cyclohexylethyl)-7-isobutyl-N-(3-methoxyphenethyl)-6-methyl-1,5,8,11-tetraoxo-13- |
| | (pyridin-2-ylmethyl)-1,2,3,4,5,6,7,8,9,10,11,12,13,14-tetradecahydronaphtho[1,2- |
| | p][1]oxa[4,7,10,14]tetraazacycloheptadecine-3-carboxamide |
| 702 | (3S,7S,13R)-13-benzyl-9-((2,3-dihydrobenzofuran-2-yl)methyl)-7-isobutyl-6-methyl-N-(2-(3- |
| | methylisoxazol-5-yl)ethyl)-1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 703 | (3S,7S,13R)-13-(benzo[b]thiophen-3-ylmethyl)-7-isobutyl-6-methyl-N-(2-(3-methylisoxazol-5-yl)ethyl)- |
| | 1,5,8,11-tetraoxo-9-(2-((S)-tetrahydro-2H-pyran-2-yl)ethyl)-1,2,3,4,5,6,7,8,9,10,11,12,13,14- |
| | tetradecahydro-[1]oxa[4,7,10,14]tetraazacycloheptadecino[17,16-c]quinoline-3-carboxamide |
| 704 | (3S,7S,13R)-13-benzyl-9-(((S)-chroman-3-yl)methyl)-7-isobutyl-6-methyl-N-(2-(3-methylisoxazol-5- |
| | yl)ethyl)-1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 705 | (3S,7S,13R)-13-benzyl-9-(((R)-chroman-3-yl)methyl)-7-isobutyl-6-methyl-N-(2-(3-methylisoxazol-5- |
| | yl)ethyl)-1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 706 | (3S,7S,13R)-13-benzyl-9-(((R)-6,6-dimethyltetrahydro-2H-pyran-2-yl)methyl)-7-isobutyl-6-methyl-N-(2- |
| | (3-methylisoxazol-5-yl)ethyl)-1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 707 | (3S,7S,13R)-13-benzyl-9-(((S)-6,6-dimethyltetrahydro-2H-pyran-2-yl)methyl)-7-isobutyl-6-methyl-N-(2- |
| | (3-methylisoxazol-5-yl)ethyl)-1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 708 | (3′S,7′S,13′R)-13′-benzyl-7′-isobutyl-6′,9′-dimethyl-N-(2-(3-methylisoxazol-5-yl)ethyl)-1′,5′,8′,11′- |
| | tetraoxo-2′,3′,4′,5′,6′,7′,8′,9′,11′,12′,13′,14′-dodecahydro-1′H-spiro[cyclopentane-1,10′- |
| | [1]oxa[4,7,10,14]tetraazacycloheptadecino[17,16-c]quinoline]-3′-carboxamide |
| 709 | (3S,7S,10R,13R)-13-benzyl-10-(cyclohexylmethyl)-7-isobutyl-6,9-dimethyl-N-(2-(3-methylisoxazol-5- |
| | yl)ethyl)-1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 710 | (3S,7S,10R,13R)-13-benzyl-10-cyclopentyl-7-isobutyl-6,9-dimethyl-N-(2-(3-methylisoxazol-5-yl)ethyl)- |
| | 1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 711 | (3′S,7′S,13′R)-13′-benzyl-3,3-difluoro-7′-isobutyl-6′,9′-dimethyl-N-(2-(3-methylisoxazol-5-yl)ethyl)- |
| | 1′,5′,8′,11′-tetraoxo-2′,3′,4′,5′,6′,7′,8′,9′,11′,12′,13′,14′-dodecahydro-1′H-spiro[cyclobutane-1,10′- |
| | [1]oxa[4,7,10,14]tetraazacycloheptadecino[17,16-c]quinoline]-3′-carboxamide |
| 712 | (3S,7S,13R)-N-(2-(benzo[d][1,3]dioxol-5-yl)ethyl)-13-benzyl-9-(((R)-chroman-3-yl)methyl)-7-isobutyl-6- |
| | methyl-1,5,8,11-tetraoxo-1,2,3,4,5,6,7,8,9,10,11,12,13,14-tetradecahydronaphtho[1,2- |
| | p][1]oxa[4,7,10,14]tetraazacycloheptadecine-3-carboxamide |
| 713 | (3S,7S,10R,13R)-13-benzyl-10-((benzyloxy)methyl)-7-isobutyl-N-(3-methoxyphenethyl)-6,9-dimethyl- |
| | 1,5,8,11-tetraoxo-1,2,3,4,5,6,7,8,9,10,11,12,13,14-tetradecahydronaphtho[1,2- |
| | p][1]oxa[4,7,10,14]tetraazacycloheptadecine-3-carboxamide |
| 714 | (3S,7S,10R,13R)-13-benzyl-10-((benzyloxy)methyl)-7-isobutyl-6,9-dimethyl-N-(2-(3-methylisoxazol-5- |
| | yl)ethyl)-1,5,8,11-tetraoxo-1,2,3,4,5,6,7,8,9,10,11,12,13,14-tetradecahydronaphtho[1,2- |
| | p][1]oxa[4,7,10,14]tetraazacycloheptadecine-3-carboxamide |
| 715 | (3S,7S,10S,13R)-13-benzyl-10-((benzyloxy)methyl)-7-isobutyl-N-(3-methoxyphenethyl)-6,9-dimethyl- |
| | 1,5,8,11-tetraoxo-1,2,3,4,5,6,7,8,9,10,11,12,13,14-tetradecahydronaphtho[1,2- |
| | p][1]oxa[4,7,10,14]tetraazacycloheptadecine-3-carboxamide |
| 716 | (3S,7S,10S,13R)-13-benzyl-10-((benzyloxy)methyl)-7-isobutyl-6,9-dimethyl-N-(2-(3-methylisoxazol-5- |
| | yl)ethyl)-1,5,8,11-tetraoxo-1,2,3,4,5,6,7,8,9,10,11,12,13,14-tetradecahydronaphtho[1,2- |
| | p][1]oxa[4,7,10,14]tetraazacycloheptadecine-3-carboxamide |
| 717 | (3S,7S,10RS,13R)-13-benzyl-10-(tert-butoxymethyl)-7-isobutyl-N-(3-methoxyphenethyl)-6,9-dimethyl- |
| | 1,5,8,11-tetraoxo-1,2,3,4,5,6,7,8,9,10,11,12,13,14-tetradecahydronaphtho[1,2- |
| | p][1]oxa[4,7,10,14]tetraazacycloheptadecine-3-carboxamide |
| 718 | (3S,7S,10RS,13R)-13-benzyl-10-(hydroxymethyl)-7-isobutyl-N-(3-methoxyphenethyl)-6,9-dimethyl- |
| | 1,5,8,11-tetraoxo-1,2,3,4,5,6,7,8,9,10,11,12,13,14-tetradecahydronaphtho[1,2- |
| | p][1]oxa[4,7,10,14]tetraazacycloheptadecine-3-carboxamide |
| 719 | (3S,7S,10RS,13R)-13-benzyl-10-(tert-butoxymethyl)-7-isobutyl-6,9-dimethyl-N-(2-(3-methylisoxazol-5- |
| | yl)ethyl)-1,5,8,11-tetraoxo-1,2,3,4,5,6,7,8,9,10,11,12,13,14-tetradecahydronaphtho[1,2- |
| | p][1]oxa[4,7,10,14]tetraazacycloheptadecine-3-carboxamide |
| 720 | (3S,7S,10RS,13R)-13-benzyl-10-(hydroxymethyl)-7-isobutyl-6,9-dimethyl-N-(2-(3-methylisoxazol-5- |
| | yl)ethyl)-1,5,8,11-tetraoxo-1,2,3,4,5,6,7,8,9,10,11,12,13,14-tetradecahydronaphtho[1,2- |
| | p][1]oxa[4,7,10,14]tetraazacycloheptadecine-3-carboxamide |
| 721 | (3S,7S,10R,13R)-13-benzyl-10-cyclopentyl-20-fluoro-7-isobutyl-6,9-dimethyl-N-(2-(3-methylisoxazol-5- |
| | yl)ethyl)-1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 722 | (3S,7S,10R,13R)-10,13-dibenzyl-20-fluoro-7-isobutyl-6,9-dimethyl-N-(2-(3-methylisoxazol-5-yl)ethyl)- |
| | 1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 723 | (3S,7S,10S,13R)-10,13-dibenzyl-20-fluoro-7-isobutyl-6,9-dimethyl-N-(2-(3-methylisoxazol-5-yl)ethyl)- |
| | 1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 724 | (3R,6RS,9S,13S)-3-benzyl-6-((benzyloxy)methyl)-N-(2-(3-cyclopropyl-1,2,4-oxadiazol-5-yl)ethyl)-9- |
| | isobutyl-16-methoxy-7,10-dimethyl-5,8,11,15-tetraoxo-2,3,4,5,6,7,8,9,10,11,12,13,14,15- |
| | tetradecahydropyrido[3,4-p][1]oxa[4,7,10,14]tetraazacycloheptadecine-13-carboxamide |
| 725 | (4aR,7R,18S,22S)-7-benzyl-22-isobutyl-21-methyl-N-(2-(3-methylisoxazol-5-yl)ethyl)-5,16,20,23- |
| | tetraoxo-2,3,4,4a,5,6,7,8,16,17,18,19,20,21,22,23-hexadecahydro-1H-naphtho[1,2-p]pyrazino[2,1- |
| | f][1]oxa[4,7,10,14]tetraazacycloheptadecine-18-carboxamide |
| 726 | (4aR,7R,18S,22S)-7-benzyl-22-isobutyl-N-(3-methoxyphenethyl)-21-methyl-5,16,20,23-tetraoxo- |
| | 2,3,4,4a,5,6,7,8,16,17,18,19,20,21,22,23-hexadecahydro-1H-naphtho[1,2-p]pyrazino[2,1- |
| | f][1]oxa[4,7,10,14]tetraazacycloheptadecine-18-carboxamide |
| 727 | (9S,13S,19aR,22R)-22-benzyl-13-isobutyl-12-methyl-N-(2-(3-methylisoxazol-5-yl)ethyl)-7,11,14,20- |
| | tetraoxo-7,8,9,10,11,12,13,14,17,18,19,19a,20,21,22,23-hexadecahydro-16H- |
| | pyrazino[2′,1′:6,7][1]oxa[4,7,10,14]tetraazacycloheptadecino[17,16-c]quinoline-9-carboxamide |
| 728 | (8R,14S,18S)-8-benzyl-12-(2-cyclohexylethyl)-N-(3-((RS)-1-hydroxyethyl)phenethyl)-14-isobutyl-2,15- |
| | dimethyl-10,13,16,20-tetraoxo-7,8,9,10,11,12,13,14,15,16,17,18,19,20- |
| | tetradecahydrooxazolo[4′,5′:5,6]benzo[1,2-p][1]oxa[4,7,10,14]tetraazacycloheptadecine-18- |
| | carboxamide |
| 729 | (4aR,7R,18S,22S)-7-benzyl-22-isobutyl-N-(3-methoxyphenethyl)-21-methyl-5,16,20,23-tetraoxo-3- |
| | (phenylsulfonyl)-2,3,4,4a,5,6,7,8,16,17,18,19,20,21,22,23-hexadecahydro-1H-naphtho[1,2- |
| | p]pyrazino[2,1-f][1]oxa[4,7,10,14]tetraazacycloheptadecine-18-carboxamide |
| 730 | (4aR,7R,18S,22S)-7-benzyl-22-isobutyl-N-(3-methoxyphenethyl)-21-methyl-3-((3- |
| | morpholinopropyl)sulfonyl)-5,16,20,23-tetraoxo-2,3,4,4a,5,6,7,8,16,17,18,19,20,21,22,23- |
| | hexadecahydro-1H-naphtho[1,2-p]pyrazino[2,1-f][1]oxa[4,7,10,14]tetraazacycloheptadecine-18- |
| | carboxamide |
| 731 | (4aR,7R,18S,22S)-7-benzyl-22-isobutyl-N-(3-methoxyphenethyl)-21-methyl-3-(methylsulfonyl)- |
| | 5,16,20,23-tetraoxo-2,3,4,4a,5,6,7,8,16,17,18,19,20,21,22,23-hexadecahydro-1H-naphtho[1,2- |
| | p]pyrazino[2,1-f][1]oxa[4,7,10,14]tetraazacycloheptadecine-18-carboxamide |
| 732 | (4aR,7R,18S,22S)-7-benzyl-22-isobutyl-N-(3-methoxyphenethyl)-21-methyl-5,16,20,23-tetraoxo-3-((3- |
| | (trifluoromethoxy)phenyl)sulfonyl)-2,3,4,4a,5,6,7,8,16,17,18,19,20,21,22,23-hexadecahydro-1H- |
| | naphtho[1,2-p]pyrazino[2,1-f][1]oxa[4,7,10,14]tetraazacycloheptadecine-18-carboxamide |
| 733 | (4aR,7R,18S,22S)-7-benzyl-22-isobutyl-3-((2-methoxyethyl)sulfonyl)-N-(3-methoxyphenethyl)-21- |
| | methyl-5,16,20,23-tetraoxo-2,3,4,4a,5,6,7,8,16,17,18,19,20,21,22,23-hexadecahydro-1H-naphtho[1,2- |
| | p]pyrazino[2,1-f][1]oxa[4,7,10,14]tetraazacycloheptadecine-18-carboxamide |
| 734 | (4aR,7R,18S,22S)-7-benzyl-22-isobutyl-N-(3-methoxyphenethyl)-21-methyl-5,16,20,23-tetraoxo-3- |
| | ((tetrahydro-2H-pyran-4-yl)sulfonyl)-2,3,4,4a,5,6,7,8,16,17,18,19,20,21,22,23-hexadecahydro-1H- |
| | naphtho[1,2-p]pyrazino[2,1-f][1]oxa[4,7,10,14]tetraazacycloheptadecine-18-carboxamide |
| 735 | (4aR,7R,18S,22S)-7-benzyl-3-((2-(dimethylamino)ethyl)sulfonyl)-22-isobutyl-N-(3-methoxyphenethyl)- |
| | 21-methyl-5,16,20,23-tetraoxo-2,3,4,4a,5,6,7,8,16,17,18,19,20,21,22,23-hexadecahydro-1H- |
| | naphtho[1,2-p]pyrazino[2,1-f][1]oxa[4,7,10,14]tetraazacycloheptadecine-18-carboxamide |
| 736 | (4aR,7R,18S,22S)-7-benzyl-3-(benzylsulfonyl)-22-isobutyl-N-(3-methoxyphenethyl)-21-methyl- |
| | 5,16,20,23-tetraoxo-2,3,4,4a,5,6,7,8,16,17,18,19,20,21,22,23-hexadecahydro-1H-naphtho[1,2- |
| | p]pyrazino[2,1-f][1]oxa[4,7,10,14]tetraazacycloheptadecine-18-carboxamide |
| 737 | (4aR,7R,18S,22S)-7-benzyl-22-isobutyl-N-(3-methoxyphenethyl)-21-methyl-5,16,20,23-tetraoxo-3-((2- |
| | oxo-2,3-dihydrobenzo[d]oxazol-5-yl)sulfonyl)-2,3,4,4a,5,6,7,8,16,17,18,19,20,21,22,23-hexadecahydro- |
| | 1H-naphtho[1,2-p]pyrazino[2,1-f][1]oxa[4,7,10,14]tetraazacycloheptadecine-18-carboxamide |
| 738 | (4aR,7R,18S,22S)-7-benzyl-22-isobutyl-N-(3-methoxyphenethyl)-21-methyl-5,16,20,23-tetraoxo-3- |
| | ((quinolin-8-ylmethyl)sulfonyl)-2,3,4,4a,5,6,7,8,16,17,18,19,20,21,22,23-hexadecahydro-1H- |
| | naphtho[1,2-p]pyrazino[2,1-f][1]oxa[4,7,10,14]tetraazacycloheptadecine-18-carboxamide |
| 739 | (4aR,7R,18S,22S)-7-benzyl-22-isobutyl-N-(3-methoxyphenethyl)-3-((3-methoxyphenyl)sulfonyl)-21- |
| | methyl-5,16,20,23-tetraoxo-2,3,4,4a,5,6,7,8,16,17,18,19,20,21,22,23-hexadecahydro-1H-naphtho[1,2- |
| | p]pyrazino[2,1-f][1]oxa[4,7,10,14]tetraazacycloheptadecine-18-carboxamide |
| 740 | (4aR,7R,18S,22S)-7-benzyl-3-((2-fluoro-5-methoxyphenyl)sulfonyl)-22-isobutyl-N-(3- |
| | methoxyphenethyl)-21-methyl-5,16,20,23-tetraoxo-2,3,4,4a,5,6,7,8,16,17,18,19,20,21,22,23- |
| | hexadecahydro-1H-naphtho[1,2-p]pyrazino[2,1-f][1]oxa[4,7,10,14]tetraazacycloheptadecine-18- |
| | carboxamide |
| 741 | (4aR,7R,18S,22S)-7-benzyl-22-isobutyl-N-(3-methoxyphenethyl)-21-methyl-3-((4-methyl-3,4-dihydro- |
| | 2H-benzo[b][1,4]oxazin-7-yl)sulfonyl)-5,16,20,23-tetraoxo-2,3,4,4a,5,6,7,8,16,17,18,19,20,21,22,23- |
| | hexadecahydro-1H-naphtho[1,2-p]pyrazino[2,1-f][1]oxa[4,7,10,14]tetraazacycloheptadecine-18- |
| | carboxamide |
| 742 | (4aR,7R,18S,22S)-7-benzyl-22-isobutyl-N-(3-methoxyphenethyl)-21-methyl-5,16,20,23-tetraoxo-3- |
| | (pyridin-3-ylsulfonyl)-2,3,4,4a,5,6,7,8,16,17,18,19,20,21,22,23-hexadecahydro-1H-naphtho[1,2- |
| | p]pyrazino[2,1-f][1]oxa[4,7,10,14]tetraazacycloheptadecine-18-carboxamide |
| 743 | (4aR,7R,18S,22S)-7-benzyl-22-isobutyl-N-(3-methoxyphenethyl)-3-((3-methoxypropyl)sulfonyl)-21- |
| | methyl-5,16,20,23-tetraoxo-2,3,4,4a,5,6,7,8,16,17,18,19,20,21,22,23-hexadecahydro-1H-naphtho[1,2- |
| | p]pyrazino[2,1-f][1]oxa[4,7,10,14]tetraazacycloheptadecine-18-carboxamide |
| 744 | (4aR,7R,18S,22S)-7-benzyl-22-isobutyl-N-(3-methoxyphenethyl)-3-((5-methoxypyridin-3-yl)sulfonyl)-21- |
| | methyl-5,16,20,23-tetraoxo-2,3,4,4a,5,6,7,8,16,17,18,19,20,21,22,23-hexadecahydro-1H-naphtho[1,2- |
| | p]pyrazino[2,1-f][1]oxa[4,7,10,14]tetraazacycloheptadecine-18-carboxamide |
| 745 | (4aR,7R,18S,22S)-7-benzyl-22-isobutyl-N-(3-methoxyphenethyl)-21-methyl-3-((2- |
| | morpholinoethyl)sulfonyl)-5,16,20,23-tetraoxo-2,3,4,4a,5,6,7,8,16,17,18,19,20,21,22,23- |
| | hexadecahydro-1H-naphtho[1,2-p]pyrazino[2,1-f][1]oxa[4,7,10,14]tetraazacycloheptadecine-18- |
| | carboxamide |
| 746 | (4aR,7R,18S,22S)-7-benzyl-22-isobutyl-N-(3-methoxyphenethyl)-21-methyl-5,16,20,23-tetraoxo-3- |
| | (((tetrahydro-2H-pyran-4-yl)methyl)sulfonyl)-2,3,4,4a,5,6,7,8,16,17,18,19,20,21,22,23-hexadecahydro- |
| | 1H-naphtho[1,2-p]pyrazino[2,1-f][1]oxa[4,7,10,14]tetraazacycloheptadecine-18-carboxamide |
| 747 | (4aR,7R,18S,22S)-7-benzyl-22-isobutyl-N-(3-methoxyphenethyl)-21-methyl-5,16,20,23-tetraoxo-3- |
| | (pyridin-2-ylsulfonyl)-2,3,4,4a,5,6,7,8,16,17,18,19,20,21,22,23-hexadecahydro-1H-naphtho[1,2- |
| | p]pyrazino[2,1-f][1]oxa[4,7,10,14]tetraazacycloheptadecine-18-carboxamide |
| 748 | (9S,13S,19aR,22R)-22-benzyl-13-isobutyl-12-methyl-18-((4-methyl-3,4-dihydro-2H-benzo[b][1,4]oxazin- |
| | 7-yl)sulfonyl)-N-(2-(3-methylisoxazol-5-yl)ethyl)-7,11,14,20-tetraoxo- |
| | 7,8,9,10,11,12,13,14,17,18,19,19a,20,21,22,23-hexadecahydro-16H- |
| | pyrazino[2′,11:6,7][1]oxa[4,7,10,14]tetraazacycloheptadecino[17,16-c]quinoline-9-carboxamide |
| 749 | (9S,13S,19aR,22R)-22-benzyl-13-isobutyl-12-methyl-N-(2-(3-methylisoxazol-5-yl)ethyl)-7,11,14,20- |
| | tetraoxo-18-((3-(trifluoromethoxy)phenyl)sulfonyl)-7,8,9,10,11,12,13,14,17,18,19,19a,20,21,22,23- |
| | hexadecahydro-16H-pyrazino[2′,1′:6,7][1]oxa[4,7,10,14]tetraazacycloheptadecino[17,16-c]quinoline-9- |
| | carboxamide |
| 750 | (4aR,7R,18S,22S)-3-acetyl-7-benzyl-22-isobutyl-N-(3-methoxyphenethyl)-21-methyl-5,16,20,23- |
| | tetraoxo-2,3,4,4a,5,6,7,8,16,17,18,19,20,21,22,23-hexadecahydro-1H-naphtho[1,2-p]pyrazino[2,1- |
| | f][1]oxa[4,7,10,14]tetraazacycloheptadecine-18-carboxamide |
| 751 | (4aR,7R,18S,22S)-7-benzyl-22-isobutyl-N-(3-methoxyphenethyl)-21-methyl-3-(3-methylbutanoyl)- |
| | 5,16,20,23-tetraoxo-2,3,4,4a,5,6,7,8,16,17,18,19,20,21,22,23-hexadecahydro-1H-naphtho[1,2- |
| | p]pyrazino[2,1-f][1]oxa[4,7,10,14]tetraazacycloheptadecine-18-carboxamide |
| 752 | (4aR,7R,18S,22S)-7-benzyl-22-isobutyl-N-(3-methoxyphenethyl)-3-(3-methoxypropanoyl)-21-methyl- |
| | 5,16,20,23-tetraoxo-2,3,4,4a,5,6,7,8,16,17,18,19,20,21,22,23-hexadecahydro-1H-naphtho[1,2- |
| | p]pyrazino[2,1-f][1]oxa[4,7,10,14]tetraazacycloheptadecine-18-carboxamide |
| 753 | (4aR,7R,18S,22S)-7-benzyl-22-isobutyl-N-(3-methoxyphenethyl)-21-methyl-5,16,20,23-tetraoxo-3- |
| | (pyrazine-2-carbonyl)-2,3,4,4a,5,6,7,8,16,17,18,19,20,21,22,23-hexadecahydro-1H-naphtho[1,2- |
| | p]pyrazino[2,1-f][1]oxa[4,7,10,14]tetraazacycloheptadecine-18-carboxamide |
| 754 | methyl (4aR,7R,18S,22S)-7-benzyl-22-isobutyl-18-((3-methoxyphenethyl)carbamoyl)-21-methyl- |
| | 5,16,20,23-tetraoxo-1,2,4,4a,5,6,7,8,16,17,18,19,20,21,22,23-hexadecahydro-3H-naphtho[1,2- |
| | p]pyrazino[2,1-f][1]oxa[4,7,10,14]tetraazacycloheptadecine-3-carboxylate |
| 755 | ethyl (4aR,7R,18S,22S)-7-benzyl-22-isobutyl-18-((3-methoxyphenethyl)carbamoyl)-21-methyl- |
| | 5,16,20,23-tetraoxo-1,2,4,4a,5,6,7,8,16,17,18,19,20,21,22,23-hexadecahydro-3H-naphtho[1,2- |
| | p]pyrazino[2,1-f][1]oxa[4,7,10,14]tetraazacycloheptadecine-3-carboxylate |
| 756 | tetrahydro-2H-pyran-4-yl (4aR,7R,18S,22S)-7-benzyl-22-isobutyl-18-((3-methoxyphenethyl)carbamoyl)- |
| | 21-methyl-5,16,20,23-tetraoxo-1,2,4,4a,5,6,7,8,16,17,18,19,20,21,22,23-hexadecahydro-3H- |
| | naphtho[1,2-p]pyrazino[2,1-f][1]oxa[4,7,10,14]tetraazacycloheptadecine-3-carboxylate |
| 757 | tert-butyl (((4aR,7R,18S,22S)-7-benzyl-22-isobutyl-18-((3-methoxyphenethyl)carbamoyl)-21-methyl- |
| | 5,16,20,23-tetraoxo-1,2,4,4a,5,6,7,8,16,17,18,19,20,21,22,23-hexadecahydro-3H-naphtho[1,2- |
| | p]pyrazino[2,1-f][1]oxa[4,7,10,14]tetraazacycloheptadecin-3-yl)(oxo)(phenyl)-16- |
| | sulfaneylidene)carbamate |
| 758 | (4aR,7R,18S,22S)-7-benzyl-22-isobutyl-N-(3-methoxyphenethyl)-21-methyl-5,16,20,23-tetraoxo-3- |
| | (phenylsulfonimidoyl)-2,3,4,4a,5,6,7,8,16,17,18,19,20,21,22,23-hexadecahydro-1H-naphtho[1,2- |
| | p]pyrazino[2,1-f][1]oxa[4,7,10,14]tetraazacycloheptadecine-18-carboxamide |
| 759 | (4aR,7R,18S,22S)-7-benzyl-N-(3-cyanophenethyl)-22-isobutyl-21-methyl-5,16,20,23-tetraoxo- |
| | 1,2,4,4a,5,6,7,8,16,17,18,19,20,21,22,23-hexadecahydronaphtho[1,2-p][1,4]oxazino[3,4- |
| | f][1]oxa[4,7,10,14]tetraazacycloheptadecine-18-carboxamide |
| 760 | (9S,13S,19aR,22R)-22-benzyl-13-isobutyl-9-((3-methoxyphenethyl)carbamoyl)-12-methyl-7,11,14,20- |
| | tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline 2-oxide |
| 761 | (8′R,14′S,18′S)-8′-benzyl-14′-isobutyl-2′,12′,15′-trimethyl-10′,13′,16′,20′-tetraoxo-N-(2-(3-phenylisoxazol- |
| | 5-yl)ethyl)-7′,8′,9′,10′,13′,14′,15′,16′,17′,18′,19′,20′-dodecahydro-12′H-spiro[cyclopropane-1,11′- |
| | oxazolo[4′,5′:5,6]benzo[1,2-p][1]oxa[4,7,10,14]tetraazacycloheptadecine]-18′-carboxamide |
| 762 | (8′R,14′S,18′S)-8′-benzyl-14′-isobutyl-N-(2-(3-isopropylisoxazol-5-yl)ethyl)-2′,12′,15′-trimethyl- |
| | 10′,13′,16′,20′-tetraoxo-7′,8′,9′,10′,13′,14′,15′,16′,17′,18′,19′,20′-dodecahydro-12′H-spiro[cyclopropane- |
| | 1,11′-oxazolo[4′,5′:5,6]benzo[1,2-p][1]oxa[4,7,10,14]tetraazacycloheptadecine]-18′-carboxamide |
| 763 | (3′S,7′S,13′R)-13′-benzyl-N-(2-(3-benzyl-1,2,4-oxadiazol-5-yl)ethyl)-7′-isobutyl-6′,9′-dimethyl-1′,5′,8′,11′- |
| | tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 764 | (3′S,7′S,13′R)-13′-benzyl-N-(2-(3-(cyclopropylmethyl)-1,2,4-oxadiazol-5-yl)ethyl)-7′-isobutyl-6′,9′- |
| | dimethyl-1′,5′,8′,11′-tetraoxo-2′,3′,4′,5′,6′,7′,8′,9′,11′,12′,13′,14′-dodecahydro-1′H-spiro[cyclopropane- |
| | 1,10′-[1]oxa[4,7,10,14]tetraazacycloheptadecino[17,16-c]quinoline]-3′-carboxamide |
| 765 | (3R,6R,9S,13S)-3-benzyl-N-(2-(4-fluoro-3-methoxyisoxazol-5-yl)ethyl)-9-isobutyl-16-methoxy-6,7,10- |
| | trimethyl-5,8,11,15-tetraoxo-2,3,4,5,6,7,8,9,10,11,12,13,14,15-tetradecahydropyrido[3,4- |
| | p][1]oxa[4,7,10,14]tetraazacycloheptadecine-13-carboxamide |
| 766 | (7S,11S,17aR,20R)-20-benzyl-N-(2-(4-fluoro-3-methoxyisoxazol-5-yl)ethyl)-11-isobutyl-4-methoxy-2,10- |
| | dimethyl-5,9,12,18-tetraoxo-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 767 | (7S,11S,17aR,20R)-20-benzyl-N-(2-(4-fluoro-3-methoxyisoxazol-5-yl)ethyl)-11-isobutyl-4-methoxy-10- |
| | methyl-5,9,12,18-tetraoxo-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 768 | (3R,6R,9S,13S)-3-benzyl-9-isobutyl-16-methoxy-N-(3-methoxyphenethyl)-6,7,10-trimethyl-5,8,11,15- |
| | tetraoxo-2,3,4,5,6,7,8,9,10,11,12,13,14,15-tetradecahydropyrido[3,4- |
| | p][1]oxa[4,7,10,14]tetraazacycloheptadecine-13-carboxamide |
| 769 | (9S,13S,19aR,22R)-22-benzyl-5-fluoro-13-isobutyl-N-(2-(3-methoxyisoxazol-5-yl)ethyl)-12-methyl- |
| | 7,11,14,20-tetraoxo-7,8,9,10,11,12,13,14,17,18,19,19a,20,21,22,23-hexadecahydro-16H- |
| | pyrido[2′,11:6,7][1]oxa[4,7,10,14]tetraazacycloheptadecino[16,17-f]quinoline-9-carboxamide |
| 770 | (9S,13S,19aS,22R)-22-benzyl-5-fluoro-13-isobutyl-N-(2-(3-methoxyisoxazol-5-yl)ethyl)-12-methyl- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[16,17-f]quinoline-9-carboxamide |
| 771 | (3R,6R,9S,13S)-3-benzyl-9-isobutyl-16-methoxy-N-(2-(3-methoxyisoxazol-5-yl)ethyl)-6,7,10-trimethyl- |
| | 5,8,11,15-tetraoxo-2,3,4,5,6,7,8,9,10,11,12,13,14,15-tetradecahydropyrido[3,4- |
| | p][1]oxa[4,7,10,14]tetraazacycloheptadecine-13-carboxamide |
| 772 | (3R,6R,9S,13S)-3-benzyl-9-isobutyl-16-methoxy-N-(2-(3-methoxyisoxazol-5-yl)ethyl)-6,7,10,18- |
| | tetramethyl-5,8,11,15-tetraoxo-2,3,4,5,6,7,8,9,10,11,12,13,14,15-tetradecahydropyrido[3,4- |
| | p][1]oxa[4,7,10,14]tetraazacycloheptadecine-13-carboxamide |
| 773 | (3R,6R,9S,13S)-3-benzyl-6-ethyl-9-isobutyl-16-methoxy-7,10,18-trimethyl-N-(2-(3-methylisoxazol-5- |
| | yl)ethyl)-5,8,11,15-tetraoxo-2,3,4,5,6,7,8,9,10,11,12,13,14,15-tetradecahydropyrido[3,4- |
| | p][1]oxa[4,7,10,14]tetraazacycloheptadecine-13-carboxamide |
| 774 | (3R,6R,9S,13S)-3-benzyl-6-ethyl-9-isobutyl-16-methoxy-N-(3-methoxyphenethyl)-7,10,18-trimethyl- |
| | 5,8,11,15-tetraoxo-2,3,4,5,6,7,8,9,10,11,12,13,14,15-tetradecahydropyrido[3,4- |
| | p][1]oxa[4,7,10,14]tetraazacycloheptadecine-13-carboxamide |
| 775 | (3R,6R,9S,13S)-3-benzyl-6-(2,2-difluoroethyl)-9-isobutyl-16-methoxy-7,10,18-trimethyl-N-(2-(3- |
| | methylisoxazol-5-yl)ethyl)-5,8,11,15-tetraoxo-2,3,4,5,6,7,8,9,10,11,12,13,14,15- |
| | tetradecahydropyrido[3,4-p][1]oxa[4,7,10,14]tetraazacycloheptadecine-13-carboxamide |
| 776 | (3R,6R,9S,13S)-3-benzyl-9-isobutyl-16-methoxy-N-(2-(3-methoxyisoxazol-5-yl)ethyl)-7,10-dimethyl- |
| | 5,8,11,15-tetraoxo-6-(2,2,2-trifluoroethyl)-2,3,4,5,6,7,8,9,10,11,12,13,14,15-tetradecahydropyrido[3,4- |
| | p][1]oxa[4,7,10,14]tetraazacycloheptadecine-13-carboxamide |
| 777 | (9S,13S,19aS,22R)-22-benzyl-N-(2-(3-cyclopropylisoxazol-5-yl)ethyl)-5-fluoro-13-isobutyl-12-methyl- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[16,17-f]quinoline-9-carboxamide |
| 778 | (9S,13S,19aR,22R)-22-benzyl-N-(2-(4-cyclopropyl-2H-1,2,3-triazol-2-yl)ethyl)-5-fluoro-13-isobutyl-12- |
| | methyl-7,11,14,20-tetraoxo-7,8,9,10,11,12,13,14,17,18,19,19a,20,21,22,23-hexadecahydro-16H- |
| | pyrido[2′,11:6,7][1]oxa[4,7,10,14]tetraazacycloheptadecino[16,17-f]quinoline-9-carboxamide |
| 779 | (9S,13S,19aS,22R)-22-benzyl-N-(2-(4-cyclopropyl-2H-1,2,3-triazol-2-yl)ethyl)-5-fluoro-13-isobutyl-12- |
| | methyl-7,11,14,20-tetraoxo-7,8,9,10,11,12,13,14,17,18,19,19a,20,21,22,23-hexadecahydro-16H- |
| | pyrido[2′,11:6,7][1]oxa[4,7,10,14]tetraazacycloheptadecino[16,17-f]quinoline-9-carboxamide |
| 780 | (9S,13S,19aR,22R)-22-benzyl-N-(2-(3-cyclopropyl-1,2,4-oxadiazol-5-yl)ethyl)-5-fluoro-13-isobutyl-12- |
| | methyl-7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[16,17-f]quinoline-9-carboxamide |
| 781 | (9S,13S,19aR,22R)-22-benzyl-N-(2-(5-cyclopropylisoxazol-3-yl)ethyl)-5-fluoro-13-isobutyl-12-methyl- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[16,17-f]quinoline-9-carboxamide |
| 782 | (3R,6R,9S,13S)-3-benzyl-N-(2-(5-cyclopropyl-1,2,4-oxadiazol-3-yl)ethyl)-6-((3-(5-fluoropyridin-2-yl)- |
| | 1,2,4-oxadiazol-5-yl)methyl)-9-isobutyl-16-methoxy-7,10-dimethyl-5,8,11,15-tetraoxo- |
| | 2,3,4,5,6,7,8,9,10,11,12,13,14,15-tetradecahydropyrido[3,4- |
| | p][1]oxa[4,7,10,14]tetraazacycloheptadecine-13-carboxamide |
| 783 | (3R,6R,9S,13S)-3-benzyl-6-((3-(5-fluoropyridin-2-yl)-1,2,4-oxadiazol-5-yl)methyl)-9-isobutyl-16- |
| | methoxy-N-(2-(3-methoxyisoxazol-5-yl)ethyl)-7,10-dimethyl-5,8,11,15-tetraoxo- |
| | 2,3,4,5,6,7,8,9,10,11,12,13,14,15-tetradecahydropyrido[3,4- |
| | p][1]oxa[4,7,10,14]tetraazacycloheptadecine-13-carboxamide |
| 784 | (3R,6R,9S,13S)-3-benzyl-6-ethyl-9-isobutyl-16-methoxy-N-(2-(3-methoxyisoxazol-5-yl)ethyl)-7,10,18- |
| | trimethyl-5,8,11,15-tetraoxo-2,3,4,5,6,7,8,9,10,11,12,13,14,15-tetradecahydropyrido[3,4- |
| | p][1]oxa[4,7,10,14]tetraazacycloheptadecine-13-carboxamide |
| 785 | (3R,6R,9S,13S)-3-benzyl-6-(2,2-difluoroethyl)-9-isobutyl-16-methoxy-N-(2-(3-methoxyisoxazol-5- |
| | yl)ethyl)-7,10,18-trimethyl-5,8,11,15-tetraoxo-2,3,4,5,6,7,8,9,10,11,12,13,14,15- |
| | tetradecahydropyrido[3,4-p][1]oxa[4,7,10,14]tetraazacycloheptadecine-13-carboxamide |
| 786 | (3S,7S,10R,13R)-13-benzyl-20-fluoro-7-isobutyl-N-(2-(3-methoxyisoxazol-5-yl)ethyl)-6,9,10-trimethyl- |
| | 1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 787 | (3S,7S,10R,13R)-13-benzyl-N-(2-(4-cyclopropyl-2H-1,2,3-triazol-2-yl)ethyl)-20-fluoro-7-isobutyl-6,9,10- |
| | trimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 788 | (3S,7S,10R,13R)-13-benzyl-7-isobutyl-N-(3-methoxyphenethyl)-6,9,10-trimethyl-1,5,8,11-tetraoxo- |
| | 1,2,3,4,5,6,7,8,9,10,11,12,13,14-tetradecahydroimidazo[1′,2′:1,6]pyrido[2,3- |
| | p][1]oxa[4,7,10,14]tetraazacycloheptadecine-3-carboxamide |
| 789 | (3S,7S,10R,13R)-13-benzyl-7-isobutyl-N-(2-(3-methoxyisoxazol-5-yl)ethyl)-6,9,10-trimethyl-1,5,8,11- |
| | tetraoxo-1,2,3,4,5,6,7,8,9,10,11,12,13,14-tetradecahydroimidazo[1′,2′:1,6]pyrido[2,3- |
| | p][1]oxa[4,7,10,14]tetraazacycloheptadecine-3-carboxamide |
| 790 | (3S,7S,10R,13R)-13-benzyl-N-(2-(3-cyclopropylisoxazol-5-yl)ethyl)-7-isobutyl-6,9,10-trimethyl-1,5,8,11- |
| | tetraoxo-1,2,3,4,5,6,7,8,9,10,11,12,13,14-tetradecahydroimidazo[1′,2′:1,6]pyrido[2,3- |
| | p][1]oxa[4,7,10,14]tetraazacycloheptadecine-3-carboxamide |
| 791 | (3R,6R,9S,13S)-3-benzyl-9-isobutyl-16-methoxy-N-(2-(3-methoxyisoxazol-5-yl)ethyl)-7,10,18-trimethyl- |
| | 5,8,11,15-tetraoxo-6-(2,2,2-trifluoroethyl)-2,3,4,5,6,7,8,9,10,11,12,13,14,15-tetradecahydropyrido[3,4- |
| | p][1]oxa[4,7,10,14]tetraazacycloheptadecine-13-carboxamide |
| 792 | (3R,6S,9S,13S)-3-benzyl-9-isobutyl-16-methoxy-N-(2-(3-methoxyisoxazol-5-yl)ethyl)-7,10,18-trimethyl- |
| | 5,8,11,15-tetraoxo-6-(2,2,2-trifluoroethyl)-2,3,4,5,6,7,8,9,10,11,12,13,14,15-tetradecahydropyrido[3,4- |
| | p][1]oxa[4,7,10,14]tetraazacycloheptadecine-13-carboxamide |
| 793 | (9S,13S,19aS,22R)-22-benzyl-N-(2-(5-cyclopropyl-1,2,4-oxadiazol-3-yl)ethyl)-5-fluoro-13-isobutyl-12- |
| | methyl-7,11,14,20-tetraoxo-7,8,9,10,11,12,13,14,17,18,19,19a,20,21,22,23-hexadecahydro-16H- |
| | pyrido[2′,11:6,7][1]oxa[4,7,10,14]tetraazacycloheptadecino[16,17-f]quinoline-9-carboxamide |
| 794 | (3S,7S,10R,13R)-13-benzyl-N-(2-(5-cyclopropyl-1,2,4-oxadiazol-3-yl)ethyl)-20-fluoro-10-((3-(5- |
| | fluoropyridin-2-yl)-1,2,4-oxadiazol-5-yl)methyl)-7-isobutyl-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[16,17- |
| | f]quinoline-3-carboxamide |
| 795 | (3S,7S,10R,13R)-13-benzyl-20-fluoro-10-((3-(5-fluoropyridin-2-yl)-1,2,4-oxadiazol-5-yl)methyl)-7- |
| | isobutyl-N-(2-(3-methoxyisoxazol-5-yl)ethyl)-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[16,17- |
| | f]quinoline-3-carboxamide |
| 796 | (9S,13S,19aR,22R)-22-benzyl-13-isobutyl-N-(2-(3-methoxyisoxazol-5-yl)ethyl)-5,12-dimethyl- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[16,17-f]isoquinoline-9-carboxamide |
| 797 | (3S,7S,10R,13R)-13-benzyl-N-(2-(3-cyclopropylisoxazol-5-yl)ethyl)-20-fluoro-7-isobutyl-6,9,10-trimethyl- |
| | 1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 798 | (3S,7S,10R,13R)-13-benzyl-N-(2-(5-cyclopropylisoxazol-3-yl)ethyl)-20-fluoro-7-isobutyl-6,9,10-trimethyl- |
| | 1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 799 | (3S,7S,10R,13R)-13-benzyl-N-(2-(5-cyclopropyl-1,2,4-oxadiazol-3-yl)ethyl)-20-fluoro-7-isobutyl-6,9,10- |
| | trimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 800 | (3S,7S,10R,13R)-13-benzyl-N-(2-(3-cyclopropyl-1,2,4-oxadiazol-5-yl)ethyl)-20-fluoro-7-isobutyl-6,9,10- |
| | trimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 801 | (3S,7S,10R,13R)-13-benzyl-N-(2-(5-cyclopropyl-1,2,4-oxadiazol-3-yl)ethyl)-10-(2,2-difluoroethyl)-20- |
| | fluoro-7-isobutyl-6,9-dimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 802 | (3S,7S,10S,13R)-13-benzyl-N-(2-(5-cyclopropyl-1,2,4-oxadiazol-3-yl)ethyl)-10-(2,2-difluoroethyl)-20- |
| | fluoro-7-isobutyl-6,9-dimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 803 | (3S,7S,10R,13R)-13-benzyl-N-(2-(5-cyclopropyl-1,2,4-oxadiazol-3-yl)ethyl)-10-ethyl-20-fluoro-7- |
| | isobutyl-6,9-dimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 804 | (3S,7S,10R,13R)-13-benzyl-N-(2-(5-cyclopropyl-2H-tetrazol-2-yl)ethyl)-20-fluoro-10-((3-(5-fluoropyridin- |
| | 2-yl)-1,2,4-oxadiazol-5-yl)methyl)-7-isobutyl-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[16,17- |
| | f]quinoline-3-carboxamide |
| 805 | (9S,13S,19aR,22R)-22-benzyl-N-(2-(2-cyclopropyl-2H-1,2,3-triazol-4-yl)ethyl)-5-fluoro-13-isobutyl-12- |
| | methyl-7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[16,17-f]quinoline-9-carboxamide |
| 806 | (3R,6R,9S,13S)-3-benzyl-N-(2-(5-cyclopropyl-2H-tetrazol-2-yl)ethyl)-9-isobutyl-16-methoxy-7,10,18- |
| | trimethyl-5,8,11,15-tetraoxo-6-(2,2,2-trifluoroethyl)-2,3,4,5,6,7,8,9,10,11,12,13,14,15- |
| | tetradecahydropyrido[3,4-p][1]oxa[4,7,10,14]tetraazacycloheptadecine-13-carboxamide |
| 807 | (3S,7S,10R,13R)-13-benzyl-20-fluoro-10-((3-(5-fluoropyridin-2-yl)-1,2,4-oxadiazol-5-yl)methyl)-7- |
| | isobutyl-6,9-dimethyl-N-(2-(3-methylisoxazol-5-yl)ethyl)-1,5,8,11-tetraoxo- |
| | 1,2,3,4,5,6,7,8,9,10,11,12,13,14-tetradecahydro-[1]oxa[4,7,10,14]tetraazacycloheptadecino[16,17- |
| | f]quinoline-3-carboxamide |
| 808 | (3S,7S,10R,13R)-13-benzyl-N-(2-(3-cyclopropyl-1,2,4-oxadiazol-5-yl)ethyl)-20-fluoro-10-((3-(5- |
| | fluoropyridin-2-yl)-1,2,4-oxadiazol-5-yl)methyl)-7-isobutyl-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[16,17- |
| | f]quinoline-3-carboxamide |
| 809 | (3S,7S,10R,13R)-13-benzyl-20-fluoro-7-isobutyl-N-(2-(3-methoxyisoxazol-5-yl)ethyl)-6,9-dimethyl- |
| | 1,5,8,11-tetraoxo-10-((3-(trifluoromethyl)-1,2,4-oxadiazol-5-yl)methyl)-1,2,3,4,5,6,7,8,9,10,11,12,13,14- |
| | tetradecahydro-[1]oxa[4,7,10,14]tetraazacycloheptadecino[17,16-c]quinoline-3-carboxamide |
| 810 | (3S,7S,10S,13R)-13-benzyl-20-fluoro-7-isobutyl-N-(2-(3-methoxyisoxazol-5-yl)ethyl)-6,9-dimethyl- |
| | 1,5,8,11-tetraoxo-10-((3-(trifluoromethyl)-1,2,4-oxadiazol-5-yl)methyl)-1,2,3,4,5,6,7,8,9,10,11,12,13,14- |
| | tetradecahydro-[1]oxa[4,7,10,14]tetraazacycloheptadecino[17,16-c]quinoline-3-carboxamide |
| 811 | (3S,7S,10R,13R)-13-benzyl-N-(2-(5-cyclopropyl-1,2,4-oxadiazol-3-yl)ethyl)-20-fluoro-7-isobutyl-6,9- |
| | dimethyl-1,5,8,11-tetraoxo-10-((3-(trifluoromethyl)-1,2,4-oxadiazol-5-yl)methyl)- |
| | 1,2,3,4,5,6,7,8,9,10,11,12,13,14-tetradecahydro-[1]oxa[4,7,10,14]tetraazacycloheptadecino[17,16- |
| | c]quinoline-3-carboxamide |
| 812 | (3S,7S,10S,13R)-13-benzyl-N-(2-(5-cyclopropyl-1,2,4-oxadiazol-3-yl)ethyl)-20-fluoro-7-isobutyl-6,9- |
| | dimethyl-1,5,8,11-tetraoxo-10-((3-(trifluoromethyl)-1,2,4-oxadiazol-5-yl)methyl)- |
| | 1,2,3,4,5,6,7,8,9,10,11,12,13,14-tetradecahydro-[1]oxa[4,7,10,14]tetraazacycloheptadecino[17,16- |
| | c]quinoline-3-carboxamide |
| 813 | (3S,7S,10R,13R)-13-benzyl-N-(2-(5-cyclopropyl-1,2,4-oxadiazol-3-yl)ethyl)-20-fluoro-10-((3-(5- |
| | fluoropyridin-2-yl)-1,2,4-oxadiazol-5-yl)methyl)-7-isobutyl-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[17,16- |
| | c]quinoline-3-carboxamide |
| 814 | (3S,7S,10S,13R)-13-benzyl-N-(2-(5-cyclopropyl-1,2,4-oxadiazol-3-yl)ethyl)-20-fluoro-10-((3-(5- |
| | fluoropyridin-2-yl)-1,2,4-oxadiazol-5-yl)methyl)-7-isobutyl-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[17,16- |
| | c]quinoline-3-carboxamide |
| 815 | (3S,7S,10R,13R)-13-benzyl-20-fluoro-10-((3-(5-fluoropyridin-2-yl)-1,2,4-oxadiazol-5-yl)methyl)-7- |
| | isobutyl-N-(2-(3-methoxyisoxazol-5-yl)ethyl)-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[17,16- |
| | c]quinoline-3-carboxamide |
| 816 | (3S,7S,10S,13R)-13-benzyl-20-fluoro-10-((3-(5-fluoropyridin-2-yl)-1,2,4-oxadiazol-5-yl)methyl)-7- |
| | isobutyl-N-(2-(3-methoxyisoxazol-5-yl)ethyl)-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[17,16- |
| | c]quinoline-3-carboxamide |
| 817 | (3′S,7′S,13′R)-13′-benzyl-N-(2-(5-cyclopropyl-2H-tetrazol-2-yl)ethyl)-20′-fluoro-7′-isobutyl-6′,9′-dimethyl- |
| | 1′,5′,8′,11′-tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[16,17-f]quinoline]-3′-carboxamide |
| 818 | (3′S,7′S,13′R)-13′-benzyl-20′-fluoro-7′-isobutyl-N-(2-(3-methoxyisoxazol-5-yl)ethyl)-6′,9′-dimethyl- |
| | 1′,5′,8′,11′-tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[16,17-f]quinoline]-3′-carboxamide |
| 819 | (3S,7S,10R,13R)-13-benzyl-10-(2,2-difluoroethyl)-20-fluoro-7-isobutyl-N-(2-(3-methoxyisoxazol-5- |
| | yl)ethyl)-6,9-dimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 820 | (3R,6R,9S,13S)-3-benzyl-N-(2-(5-cyclopropyl-1,2,4-oxadiazol-3-yl)ethyl)-9-isobutyl-16-methoxy- |
| | 7,10,18-trimethyl-5,8,11,15-tetraoxo-6-((3-(trifluoromethyl)-1,2,4-oxadiazol-5-yl)methyl)- |
| | 2,3,4,5,6,7,8,9,10,11,12,13,14,15-tetradecahydropyrido[3,4- |
| | p][1]oxa[4,7,10,14]tetraazacycloheptadecine-13-carboxamide |
| 821 | (9S,13S,19aR,22R)-22-benzyl-5-fluoro-13-isobutyl-N-(2-(3-methoxy-1,2,4-oxadiazol-5-yl)ethyl)-12- |
| | methyl-7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[16,17-f]quinoline-9-carboxamide |
| 822 | (9S,13S,19aR,22R)-22-benzyl-13-isobutyl-N-(2-(3-methoxyisoxazol-5-yl)ethyl)-2,12-dimethyl- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[16,17-f]quinoline-9-carboxamide |
| 823 | (9S,13S,19aR,22R)-22-benzyl-N-(2-(5-cyclopropyl-2H-tetrazol-2-yl)ethyl)-13-isobutyl-5,12-dimethyl- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[16,17-f]isoquinoline-9-carboxamide |
| 824 | (3S,7S,10R,13R)-13-benzyl-7-isobutyl-6,9,10,20-tetramethyl-N-(2-(3-methylisoxazol-5-yl)ethyl)- |
| | 1,5,8,11-tetraoxo-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 825 | (3S,7S,10R,13R)-13-benzyl-7-isobutyl-N-(2-(3-methoxyisoxazol-5-yl)ethyl)-6,9,10,20-tetramethyl- |
| | 1,5,8,11-tetraoxo-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 826 | (3S,7S,10R,13R)-13-benzyl-N-(2-(3-cyclopropyl-1,2,4-oxadiazol-5-yl)ethyl)-7-isobutyl-6,9,10,20- |
| | tetramethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 827 | (3S,7S,10R,13R)-13-benzyl-10-((3,3-difluoropyrrolidin-1-yl)methyl)-20-fluoro-7-isobutyl-N-(2-(3- |
| | methoxyisoxazol-5-yl)ethyl)-6,9-dimethyl-1,5,8,11-tetraoxo-1,2,3,4,5,6,7,8,9,10,11,12,13,14- |
| | tetradecahydro-[1]oxa[4,7,10,14]tetraazacycloheptadecino[17,16-c]quinoline-3-carboxamide |
| 828 | (3R,6R,9S,13S)-3-benzyl-N-((S)-2-hydroxy-2-phenylethyl)-6,9-diisobutyl-16-methoxy-7,10,18-trimethyl- |
| | 5,8,11,15-tetraoxo-2,3,4,5,6,7,8,9,10,11,12,13,14,15-tetradecahydropyrido[3,4- |
| | p][1]oxa[4,7,10,14]tetraazacycloheptadecine-13-carboxamide |
| 829 | (9S,13S,19aR,22R)-22-benzyl-N-(2-(5-cyclopropyl-2H-tetrazol-2-yl)ethyl)-5-fluoro-13-isobutyl-12- |
| | methyl-7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[16,17-f]quinoline-9-carboxamide |
| 830 | (3′S,7′S,13′R)-13′-benzyl-20′-fluoro-7′-isobutyl-N-(2-(1-(2-methoxyethyl)-1H-pyrazol-4-yl)ethyl)-6′,9′- |
| | dimethyl-1′,5′,8′,11′-tetraoxo-2′,3′,4′,5′,6′,7′,8′,9′,11′,12′,13′,14′-dodecahydro-1′H-spiro[cyclopropane- |
| | 1,10′-[1]oxa[4,7,10,14]tetraazacycloheptadecino[17,16-c]quinoline]-3′-carboxamide |
| 831 | (7S,11S,17aR,20R)-20-benzyl-N-(2-(3-cyclopropyl-1,2,4-oxadiazol-5-yl)-2,2-difluoroethyl)-11-isobutyl-4- |
| | methoxy-10-methyl-5,9,12,18-tetraoxo-5,6,7,8,9,10,11,12,15,16,17,17a,18,19,20,21-hexadecahydro- |
| | 14H-dipyrido[2,1-f:3′,4′-p][1]oxa[4,7,10,14]tetraazacycloheptadecine-7-carboxamide |
| 832 | (7S,11S,17aR,20R)-N-(2-(2H-benzo[d][1,2,3]triazol-2-yl)ethyl)-20-benzyl-11-isobutyl-4-methoxy-10- |
| | methyl-5,9,12,18-tetraoxo-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 833 | (7S,11S,17aR,20R)-20-benzyl-N-(2-(5-cyclopropyl-2H-tetrazol-2-yl)ethyl)-11-isobutyl-4-methoxy-10- |
| | methyl-5,9,12,18-tetraoxo-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 834 | (7S,11S,17aR,20R)-20-benzyl-N-(2-(4-ethynyl-5-methyl-2H-1,2,3-triazol-2-yl)ethyl)-11-isobutyl-4- |
| | methoxy-2,10-dimethyl-5,9,12,18-tetraoxo-5,6,7,8,9,10,11,12,15,16,17,17a,18,19,20,21- |
| | hexadecahydro-14H-dipyrido[2,1-f:3′,4′-p][1]oxa[4,7,10,14]tetraazacycloheptadecine-7-carboxamide |
| 835 | (7S,11S,17aR,20R)-20-benzyl-N-(2-(5-cyclopropyl-2H-tetrazol-2-yl)ethyl)-11-isobutyl-4-methoxy-2,10- |
| | dimethyl-5,9,12,18-tetraoxo-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 836 | (7S,11S,17aR,20R)-20-benzyl-N-(2-(4-cyano-2H-1,2,3-triazol-2-yl)ethyl)-11-isobutyl-4-methoxy-2,10- |
| | dimethyl-5,9,12,18-tetraoxo-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 837 | (3S,7S,10R,13R)-13-benzyl-N-(2-(4-cyclopropyl-2H-1,2,3-triazol-2-yl)ethyl)-20-fluoro-10-((3-(5- |
| | fluoropyridin-2-yl)-1,2,4-oxadiazol-5-yl)methyl)-7-isobutyl-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[17,16- |
| | c]quinoline-3-carboxamide |
| 838 | (3′S,7′S,13′R)-13′-benzyl-N-(2-(5-cyclopropyl-2H-tetrazol-2-yl)ethyl)-3,3,20′-trifluoro-7′-isobutyl-6′,9′- |
| | dimethyl-1′,5′,8′,11′-tetraoxo-2′,3′,4′,5′,6′,7′,8′,9′,11′,12′,13′,14′-dodecahydro-1′H-spiro[cyclobutane-1,10′- |
| | [1]oxa[4,7,10,14]tetraazacycloheptadecino[17,16-c]quinoline]-3′-carboxamide |
| 839 | (3′S,7′S,13′R)-13′-benzyl-3,3,20′-trifluoro-7′-isobutyl-N-(2-(3-methoxyisoxazol-5-yl)ethyl)-6′,9′-dimethyl- |
| | 1′,5′,8′,11′-tetraoxo-2′,3′,4′,5′,6′,7′,8′,9′,11′,12′,13′,14′-dodecahydro-1′H-spiro[cyclobutane-1,10′- |
| | [1]oxa[4,7,10,14]tetraazacycloheptadecino[17,16-c]quinoline]-3′-carboxamide |
| 840 | (3S,7S,10R,13R)-N-(2-(2H-benzo[d][1,2,3]triazol-2-yl)ethyl)-13-benzyl-20-fluoro-10-((3-(5-fluoropyridin- |
| | 2-yl)-1,2,4-oxadiazol-5-yl)methyl)-7-isobutyl-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[17,16- |
| | c]quinoline-3-carboxamide |
| 841 | (3′S,7′S,13′R)-13′-benzyl-N-(2-(3-cyclopropyl-1,2,4-oxadiazol-5-yl)ethyl)-3,3,20′-trifluoro-7′-isobutyl-6′,9′- |
| | dimethyl-1′,5′,8′,11′-tetraoxo-2′,3′,4′,5′,6′,7′,8′,9′,11′,12′,13′,14′-dodecahydro-1′H-spiro[cyclobutane-1,10′- |
| | [1]oxa[4,7,10,14]tetraazacycloheptadecino[17,16-c]quinoline]-3′-carboxamide |
| 842 | (3S,7S,10R,13R)-13-benzyl-20-fluoro-10-((3-(5-fluoropyridin-2-yl)-1,2,4-oxadiazol-5-yl)methyl)-N-((S)-2- |
| | hydroxy-2-phenylethyl)-7-isobutyl-6,9-dimethyl-1,5,8,11-tetraoxo-1,2,3,4,5,6,7,8,9,10,11,12,13,14- |
| | tetradecahydro-[1]oxa[4,7,10,14]tetraazacycloheptadecino[17,16-c]quinoline-3-carboxamide |
| 843 | (3S,7S,10R,13R)-13-benzyl-20-fluoro-10-((3-(5-fluoropyridin-2-yl)-1,2,4-oxadiazol-5-yl)methyl)-7- |
| | isobutyl-N-(2-(6-methoxypyridin-3-yl)ethyl)-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[17,16- |
| | c]quinoline-3-carboxamide |
| 844 | (3S,7S,10R,13R)-N-(2-([1,2,4]triazolo[1,5-a]pyrimidin-2-yl)ethyl)-13-benzyl-20-fluoro-10-((3-(5- |
| | fluoropyridin-2-yl)-1,2,4-oxadiazol-5-yl)methyl)-7-isobutyl-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[17,16- |
| | c]quinoline-3-carboxamide |
| 845 | (3S,7S,10R,13R)-13-benzyl-20-fluoro-10-((3-(5-fluoropyridin-2-yl)-1,2,4-oxadiazol-5-yl)methyl)-7- |
| | isobutyl-N-(3-methoxypropyl)-6,9-dimethyl-1,5,8,11-tetraoxo-1,2,3,4,5,6,7,8,9,10,11,12,13,14- |
| | tetradecahydro-[1]oxa[4,7,10,14]tetraazacycloheptadecino[17,16-c]quinoline-3-carboxamide |
| 846 | (3S,7S,10R,13R)-13-benzyl-20-fluoro-10-((3-(5-fluoropyridin-2-yl)-1,2,4-oxadiazol-5-yl)methyl)-7- |
| | isobutyl-6,9-dimethyl-1,5,8,11-tetraoxo-N-(3-(trifluoromethoxy)propyl)-1,2,3,4,5,6,7,8,9,10,11,12,13,14- |
| | tetradecahydro-[1]oxa[4,7,10,14]tetraazacycloheptadecino[17,16-c]quinoline-3-carboxamide |
| 847 | (3S,7S,10R,13R)-13-benzyl-20-fluoro-10-((3-(5-fluoropyridin-2-yl)-1,2,4-oxadiazol-5-yl)methyl)-7- |
| | isobutyl-N-(2-(5-methoxypyridin-2-yl)ethyl)-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[17,16- |
| | c]quinoline-3-carboxamide |
| 848 | (3R,6R,9S,13S)-3-benzyl-N-((S)-2-hydroxy-2-phenylethyl)-9-isobutyl-16-methoxy-7,10,18-trimethyl- |
| | 5,8,11,15-tetraoxo-6-((3-(trifluoromethyl)-1,2,4-oxadiazol-5-yl)methyl)-2,3,4,5,6,7,8,9,10,11,12,13,14,15- |
| | tetradecahydropyrido[3,4-p][1]oxa[4,7,10,14]tetraazacycloheptadecine-13-carboxamide |
| 849 | (3R,6R,9S,13S)-3-benzyl-N-(2-(3-cyclopropyl-1,2,4-oxadiazol-5-yl)ethyl)-9-isobutyl-16-methoxy- |
| | 7,10,18-trimethyl-5,8,11,15-tetraoxo-6-((3-(trifluoromethyl)-1,2,4-oxadiazol-5-yl)methyl)- |
| | 2,3,4,5,6,7,8,9,10,11,12,13,14,15-tetradecahydropyrido[3,4- |
| | p][1]oxa[4,7,10,14]tetraazacycloheptadecine-13-carboxamide |
| 850 | (7S,11S,17aR,20R)-20-benzyl-N-(3-(difluoromethoxy)propyl)-11-isobutyl-4-methoxy-10-methyl- |
| | 5,9,12,18-tetraoxo-5,6,7,8,9,10,11,12,15,16,17,17a,18,19,20,21-hexadecahydro-14H-dipyrido[2,1- |
| | f:3′,4′-p][1]oxa[4,7,10,14]tetraazacycloheptadecine-7-carboxamide |
| 851 | (3S,7S,10R,13R)-13-benzyl-N-(2-(5-cyclopropyl-1,2,4-oxadiazol-3-yl)ethyl)-20-fluoro-7-isobutyl-6,9- |
| | dimethyl-10-(2-(3-methyl-1,2,4-oxadiazol-5-yl)ethyl)-1,5,8,11-tetraoxo-1,2,3,4,5,6,7,8,9,10,11,12,13,14- |
| | tetradecahydro-[1]oxa[4,7,10,14]tetraazacycloheptadecino[17,16-c]quinoline-3-carboxamide |
| 852 | (9S,13S,19aR,22R)-22-benzyl-18-(5-fluoropyridin-2-yl)-13-isobutyl-12-methyl-N-(2-(3-methylisoxazol-5- |
| | yl)ethyl)-7,11,14,20-tetraoxo-7,8,9,10,11,12,13,14,17,18,19,19a,20,21,22,23-hexadecahydro-16H- |
| | pyrazino[2′,11:6,7][1]oxa[4,7,10,14]tetraazacycloheptadecino[17,16-c]quinoline-9-carboxamide |
| 853 | (9S,13S,19aR,22R)-22-benzyl-18-(5-fluoropyridin-2-yl)-13-isobutyl-N-(3-methoxyphenethyl)-12-methyl- |
| | 7,11,14,20-tetraoxo-7,8,9,10,11,12,13,14,17,18,19,19a,20,21,22,23-hexadecahydro-16H- |
| | pyrazino[2′,1′:6,7][1]oxa[4,7,10,14]tetraazacycloheptadecino[17,16-c]quinoline-9-carboxamide |
| 854 | (3R,6R,9S,13S)-3-benzyl-6-((4,4-difluorocyclohexyl)methyl)-9-isobutyl-16-methoxy-N-(2-(3- |
| | methoxyisoxazol-5-yl)ethyl)-7,10-dimethyl-5,8,11,15-tetraoxo-2,3,4,5,6,7,8,9,10,11,12,13,14,15- |
| | tetradecahydropyrido[3,4-p][1]oxa[4,7,10,14]tetraazacycloheptadecine-13-carboxamide |
| 855 | (3R,6R,9S,13S)-3-benzyl-9-isobutyl-16-methoxy-7,10-dimethyl-N-(2-(3-methylisoxazol-5-yl)ethyl)- |
| | 5,8,11,15-tetraoxo-6-(2-phenoxyethyl)-2,3,4,5,6,7,8,9,10,11,12,13,14,15-tetradecahydropyrido[3,4- |
| | p][1]oxa[4,7,10,14]tetraazacycloheptadecine-13-carboxamide |
| 856 | (3R,6R,9S,13S)-3-benzyl-9-isobutyl-16-methoxy-N-(2-(3-methoxyisoxazol-5-yl)ethyl)-7,10-dimethyl- |
| | 5,8,11,15-tetraoxo-6-(2-phenoxyethyl)-2,3,4,5,6,7,8,9,10,11,12,13,14,15-tetradecahydropyrido[3,4- |
| | p][1]oxa[4,7,10,14]tetraazacycloheptadecine-13-carboxamide |
| 857 | (3R,6R,9S,13S)-3-benzyl-6-(cyclobutoxymethyl)-N-(2-(5-cyclopropyl-1,2,4-oxadiazol-3-yl)ethyl)-9- |
| | isobutyl-16-methoxy-7,10-dimethyl-5,8,11,15-tetraoxo-2,3,4,5,6,7,8,9,10,11,12,13,14,15- |
| | tetradecahydropyrido[3,4-p][1]oxa[4,7,10,14]tetraazacycloheptadecine-13-carboxamide |
| 858 | (3R,6R,9S,13S)-3-benzyl-6-(2-(cyclohexyloxy)ethyl)-9-isobutyl-16-methoxy-7,10-dimethyl-N-(2-(3- |
| | methylisoxazol-5-yl)ethyl)-5,8,11,15-tetraoxo-2,3,4,5,6,7,8,9,10,11,12,13,14,15- |
| | tetradecahydropyrido[3,4-p][1]oxa[4,7,10,14]tetraazacycloheptadecine-13-carboxamide |
| 859 | (3R,6R,9S,13S)-3-benzyl-N-(2-(5-cyclopropyl-1,2,4-oxadiazol-3-yl)ethyl)-9-isobutyl-16-methoxy-7,10- |
| | dimethyl-5,8,11,15-tetraoxo-6-((3-phenyl-1,2,4-oxadiazol-5-yl)methyl)- |
| | 2,3,4,5,6,7,8,9,10,11,12,13,14,15-tetradecahydropyrido[3,4- |
| | p][1]oxa[4,7,10,14]tetraazacycloheptadecine-13-carboxamide |
| 860 | (7S,11S,14R,17R)-17-benzyl-11-isobutyl-4-methoxy-N-(3-methoxyphenethyl)-2,10,13,14-tetramethyl- |
| | 5,9,12,15-tetraoxo-5,6,7,8,9,10,11,12,13,14,15,16,17,18-tetradecahydropyrimido[5,4- |
| | p][1]oxa[4,7,10,14]tetraazacycloheptadecine-7-carboxamide |
| 861 | (3R,6R,9S,13S)-3-benzyl-N-(2-(5-cyclopropyl-1,2,4-oxadiazol-3-yl)ethyl)-9-isobutyl-16-methoxy-6-((3- |
| | methoxyisoxazol-5-yl)methyl)-7,10-dimethyl-5,8,11,15-tetraoxo-2,3,4,5,6,7,8,9,10,11,12,13,14,15- |
| | tetradecahydropyrido[3,4-p][1]oxa[4,7,10,14]tetraazacycloheptadecine-13-carboxamide |
| 862 | (7S,11S,14R,17R)-17-benzyl-N-(2-(3-cyclopropylisoxazol-5-yl)ethyl)-11-isobutyl-4-methoxy-2,10,13,14- |
| | tetramethyl-5,9,12,15-tetraoxo-5,6,7,8,9,10,11,12,13,14,15,16,17,18-tetradecahydropyrimido[5,4- |
| | p][1]oxa[4,7,10,14]tetraazacycloheptadecine-7-carboxamide |
| 863 | (7S,11S,18aR,21R)-21-benzyl-N-(2-(5-cyclopropyl-1,2,4-oxadiazol-3-yl)ethyl)-11-isobutyl-4-methoxy- |
| | 10-methyl-5,9,12,19-tetraoxo-5,6,7,8,9,10,11,12,14,15,16,17,18,18a,19,20,21,22- |
| | octadecahydroazepino[2,1-f]pyrido[3,4-p][1]oxa[4,7,10,14]tetraazacycloheptadecine-7-carboxamide |
| 864 | (3R,6R,9S,13S)-3-benzyl-N-(2-(5-cyclopropyl-1,2,4-oxadiazol-3-yl)ethyl)-6-(2-(3-cyclopropyl-1,2,4- |
| | oxadiazol-5-yl)ethyl)-9-isobutyl-16-methoxy-7,10-dimethyl-5,8,11,15-tetraoxo- |
| | 2,3,4,5,6,7,8,9,10,11,12,13,14,15-tetradecahydropyrido[3,4- |
| | p][1]oxa[4,7,10,14]tetraazacycloheptadecine-13-carboxamide |
| 865 | (3R,6R,9S,13S)-3-benzyl-6-(tert-butyl)-9-isobutyl-16-methoxy-N-(3-methoxyphenethyl)-7,10,18- |
| | trimethyl-5,8,11,15-tetraoxo-2,3,4,5,6,7,8,9,10,11,12,13,14,15-tetradecahydropyrido[3,4- |
| | p][1]oxa[4,7,10,14]tetraazacycloheptadecine-13-carboxamide |
| 866 | (3R,6R,9S,13S)-3-benzyl-6-(tert-butyl)-9-isobutyl-16-methoxy-7,10,18-trimethyl-N-(2-(3-methylisoxazol- |
| | 5-yl)ethyl)-5,8,11,15-tetraoxo-2,3,4,5,6,7,8,9,10,11,12,13,14,15-tetradecahydropyrido[3,4- |
| | p][1]oxa[4,7,10,14]tetraazacycloheptadecine-13-carboxamide |
| 867 | (3R,6R,9S,13S)-3-benzyl-N-(2-(5-cyclopropyl-1,2,4-oxadiazol-3-yl)ethyl)-9-isobutyl-16-methoxy-6-((3- |
| | (4-(2-methoxyethyl)phenyl)-1,2,4-oxadiazol-5-yl)methyl)-7,10-dimethyl-5,8,11,15-tetraoxo- |
| | 2,3,4,5,6,7,8,9,10,11,12,13,14,15-tetradecahydropyrido[3,4- |
| | p][1]oxa[4,7,10,14]tetraazacycloheptadecine-13-carboxamide |
| 868 | (3R,6R,9S,13S)-3-benzyl-9-isobutyl-16-methoxy-6-((3-(4-(2-methoxyethyl)phenyl)-1,2,4-oxadiazol-5- |
| | yl)methyl)-N-(2-(3-methoxyisoxazol-5-yl)ethyl)-7,10-dimethyl-5,8,11,15-tetraoxo- |
| | 2,3,4,5,6,7,8,9,10,11,12,13,14,15-tetradecahydropyrido[3,4- |
| | p][1]oxa[4,7,10,14]tetraazacycloheptadecine-13-carboxamide |
| 869 | (7S,11S,17aR,20R)-20-benzyl-11-isobutyl-2,4-dimethoxy-10-methyl-N-(2-(3-methylisoxazol-5-yl)ethyl)- |
| | 5,9,12,18-tetraoxo-5,6,7,8,9,10,11,12,15,16,17,17a,18,19,20,21-hexadecahydro-14H-dipyrido[2,1- |
| | f:3′,4′-p][1]oxa[4,7,10,14]tetraazacycloheptadecine-7-carboxamide |
| 870 | (7S,11S,17aR,20R)-20-benzyl-N-(2-(3-cyclopropylisoxazol-5-yl)ethyl)-11-isobutyl-2,4-dimethoxy-10- |
| | methyl-5,9,12,18-tetraoxo-5,6,7,8,9,10,11,12,15,16,17,17a,18,19,20,21-hexadecahydro-14H- |
| | dipyrido[2,1-f:3′,4′-p][1]oxa[4,7,10,14]tetraazacycloheptadecine-7-carboxamide |
| 871 | (3R,6R,9S,13S)-3-benzyl-N-(2-(4-cyclopropyl-2H-1,2,3-triazol-2-yl)ethyl)-9-isobutyl-16-methoxy-6-((3- |
| | (4-(2-methoxyethyl)phenyl)-1,2,4-oxadiazol-5-yl)methyl)-7,10-dimethyl-5,8,11,15-tetraoxo- |
| | 2,3,4,5,6,7,8,9,10,11,12,13,14,15-tetradecahydropyrido[3,4- |
| | p][1]oxa[4,7,10,14]tetraazacycloheptadecine-13-carboxamide |
| 872 | (3′S,7′S,13′R)-13′-benzyl-7′-isobutyl-N-(2-(3-methoxy-1,2,4-oxadiazol-5-yl)ethyl)-6′,9′-dimethyl- |
| | 1′,5′,8′,11′-tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[17,16-c]quinoline]-3′-carboxamide |
| 873 | (3R,6R,9S,13S)-3-benzyl-9-isobutyl-16-methoxy-N-(3-methoxyphenethyl)-7,10,18-trimethyl-6- |
| | neopentyl-5,8,11,15-tetraoxo-2,3,4,5,6,7,8,9,10,11,12,13,14,15-tetradecahydropyrido[3,4- |
| | p][1]oxa[4,7,10,14]tetraazacycloheptadecine-13-carboxamide |
| 874 | (3R,6R,9S,13S)-3-benzyl-9-isobutyl-16-methoxy-7,10,18-trimethyl-N-(2-(3-methylisoxazol-5-yl)ethyl)- |
| | 5,8,11,15-tetraoxo-6-phenethyl-2,3,4,5,6,7,8,9,10,11,12,13,14,15-tetradecahydropyrido[3,4- |
| | p][1]oxa[4,7,10,14]tetraazacycloheptadecine-13-carboxamide |
| 875 | (3R,6R,9S,13S)-3-benzyl-9-isobutyl-16-methoxy-N-(3-methoxyphenethyl)-7,10,18-trimethyl-5,8,11,15- |
| | tetraoxo-6-phenethyl-2,3,4,5,6,7,8,9,10,11,12,13,14,15-tetradecahydropyrido[3,4- |
| | p][1]oxa[4,7,10,14]tetraazacycloheptadecine-13-carboxamide |
| 876 | (3R,6R,9S,13S)-3-benzyl-9-isobutyl-16-methoxy-7,10,18-trimethyl-N-(2-(3-methylisoxazol-5-yl)ethyl)-6- |
| | neopentyl-5,8,11,15-tetraoxo-2,3,4,5,6,7,8,9,10,11,12,13,14,15-tetradecahydropyrido[3,4- |
| | p][1]oxa[4,7,10,14]tetraazacycloheptadecine-13-carboxamide |
| 877 | (3R,6R,9S,13S)-3-benzyl-6-(2-ethoxyethyl)-9-isobutyl-16-methoxy-N-(2-(3-methoxyisoxazol-5-yl)ethyl)- |
| | 7,10,18-trimethyl-5,8,11,15-tetraoxo-2,3,4,5,6,7,8,9,10,11,12,13,14,15-tetradecahydropyrido[3,4- |
| | p][1]oxa[4,7,10,14]tetraazacycloheptadecine-13-carboxamide |
| 878 | (3R,6R,9S,13S)-3-benzyl-9-isobutyl-16-methoxy-N-(2-(3-methoxyisoxazol-5-yl)ethyl)-7,10,18-trimethyl- |
| | 6-neopentyl-5,8,11,15-tetraoxo-2,3,4,5,6,7,8,9,10,11,12,13,14,15-tetradecahydropyrido[3,4- |
| | p][1]oxa[4,7,10,14]tetraazacycloheptadecine-13-carboxamide |
| 879 | (3S,7S,10R,13R)-13-benzyl-N-(2-(5-cyclopropyl-1,2,4-oxadiazol-3-yl)ethyl)-20-fluoro-7-isobutyl-10-((3- |
| | methoxyisoxazol-5-yl)methyl)-6,9-dimethyl-1,5,8,11-tetraoxo-1,2,3,4,5,6,7,8,9,10,11,12,13,14- |
| | tetradecahydro-[1]oxa[4,7,10,14]tetraazacycloheptadecino[16,17-f]quinoline-3-carboxamide |
| 880 | (3S,7S,10R,13R)-13-benzyl-20-fluoro-7-isobutyl-N-(2-(3-methoxyisoxazol-5-yl)ethyl)-10-((3- |
| | methoxyisoxazol-5-yl)methyl)-6,9-dimethyl-1,5,8,11-tetraoxo-1,2,3,4,5,6,7,8,9,10,11,12,13,14- |
| | tetradecahydro-[1]oxa[4,7,10,14]tetraazacycloheptadecino[16,17-f]quinoline-3-carboxamide |
| 881 | (3R,6R,9S,13S)-3-benzyl-6-(2-(3-(tert-butyl)-1,2,4-oxadiazol-5-yl)ethyl)-N-(2-(5-cyclopropyl-1,2,4- |
| | oxadiazol-3-yl)ethyl)-9-isobutyl-16-methoxy-7,10-dimethyl-5,8,11,15-tetraoxo- |
| | 2,3,4,5,6,7,8,9,10,11,12,13,14,15-tetradecahydropyrido[3,4- |
| | p][1]oxa[4,7,10,14]tetraazacycloheptadecine-13-carboxamide |
| 882 | (3R,6R,9S,13S)-3-benzyl-N-(2-(5-cyclopropyl-1,2,4-oxadiazol-3-yl)ethyl)-9-isobutyl-16-methoxy-7,10- |
| | dimethyl-5,8,11,15-tetraoxo-6-((4-phenyl-1H-1,2,3-triazol-1-yl)methyl)- |
| | 2,3,4,5,6,7,8,9,10,11,12,13,14,15-tetradecahydropyrido[3,4- |
| | p][1]oxa[4,7,10,14]tetraazacycloheptadecine-13-carboxamide |
| 883 | (3R,6R,9S,13S)-3-benzyl-6-(2-(3-cyclopentyl-1,2,4-oxadiazol-5-yl)ethyl)-N-(2-(5-cyclopropyl-1,2,4- |
| | oxadiazol-3-yl)ethyl)-9-isobutyl-16-methoxy-7,10-dimethyl-5,8,11,15-tetraoxo- |
| | 2,3,4,5,6,7,8,9,10,11,12,13,14,15-tetradecahydropyrido[3,4- |
| | p][1]oxa[4,7,10,14]tetraazacycloheptadecine-13-carboxamide |
| 884 | (3R,6R,9S,13S)-3-benzyl-6-(2-(3-cyclopentyl-1,2,4-oxadiazol-5-yl)ethyl)-9-isobutyl-16-methoxy-N-(2-(3- |
| | methoxyisoxazol-5-yl)ethyl)-7,10-dimethyl-5,8,11,15-tetraoxo-2,3,4,5,6,7,8,9,10,11,12,13,14,15- |
| | tetradecahydropyrido[3,4-p][1]oxa[4,7,10,14]tetraazacycloheptadecine-13-carboxamide |
| 885 | (3S,7S,10R,13R)-13-benzyl-N-(2-(5-cyclopropyl-1,2,4-oxadiazol-3-yl)ethyl)-20-fluoro-10-((4-(5- |
| | fluoropyridin-2-yl)-1H-1,2,3-triazol-1-yl)methyl)-7-isobutyl-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[17,16- |
| | c]quinoline-3-carboxamide |
| 886 | (3S,7S,10R,13R)-13-benzyl-10-((3-(tert-butyl)isoxazol-5-yl)methyl)-N-(2-(5-cyclopropyl-1,2,4-oxadiazol- |
| | 3-yl)ethyl)-20-fluoro-7-isobutyl-6,9-dimethyl-1,5,8,11-tetraoxo-1,2,3,4,5,6,7,8,9,10,11,12,13,14- |
| | tetradecahydro-[1]oxa[4,7,10,14]tetraazacycloheptadecino[17,16-c]quinoline-3-carboxamide |
| 887 | (3S,7S,10R,13R)-13-benzyl-N-(2-(5-cyclopropyl-1,2,4-oxadiazol-3-yl)ethyl)-20-fluoro-10-(2-(3-(5- |
| | fluoropyridin-2-yl)-1,2,4-oxadiazol-5-yl)ethyl)-7-isobutyl-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[17,16- |
| | c]quinoline-3-carboxamide |
| 888 | (9S,13S,19aR,22R)-22-benzyl-N-(2-(5-cyclopropyl-2H-tetrazol-2-yl)ethyl)-13-isobutyl-2,12-dimethyl- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[16,17-f]quinoline-9-carboxamide |
| 889 | (3S,7S,10R,13R)-13-benzyl-N-(2-(5-cyclopropyl-1,2,4-oxadiazol-3-yl)ethyl)-7-isobutyl-6,9,20-trimethyl- |
| | 1,5,8,11-tetraoxo-10-((3-(trifluoromethyl)-1,2,4-oxadiazol-5-yl)methyl)-1,2,3,4,5,6,7,8,9,10,11,12,13,14- |
| | tetradecahydro-[1]oxa[4,7,10,14]tetraazacycloheptadecino[16,17-f]isoquinoline-3-carboxamide |
| 890 | (3S,7S,10R,13R)-13-benzyl-N-(2-(5-cyclopropyl-2H-tetrazol-2-yl)ethyl)-7-isobutyl-6,9,20-trimethyl- |
| | 1,5,8,11-tetraoxo-10-((3-(trifluoromethyl)-1,2,4-oxadiazol-5-yl)methyl)-1,2,3,4,5,6,7,8,9,10,11,12,13,14- |
| | tetradecahydro-[1]oxa[4,7,10,14]tetraazacycloheptadecino[16,17-f]isoquinoline-3-carboxamide |
| 891 | (3S,7S,10R,13R)-13-benzyl-N-((S)-2-(3-cyclopropyl-1,2,4-oxadiazol-5-yl)-2-hydroxyethyl)-20-fluoro-10- |
| | ((3-(5-fluoropyridin-2-yl)-1,2,4-oxadiazol-5-yl)methyl)-7-isobutyl-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[16,17- |
| | f]quinoline-3-carboxamide |
| 892 | (3S,7S,10R,13R)-13-benzyl-N-((R)-2-(3-cyclopropyl-1,2,4-oxadiazol-5-yl)-2-hydroxyethyl)-20-fluoro-10- |
| | ((3-(5-fluoropyridin-2-yl)-1,2,4-oxadiazol-5-yl)methyl)-7-isobutyl-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[16,17- |
| | f]quinoline-3-carboxamide |
| 893 | (3S,7S,10R,13R)-13-benzyl-N-(2-(5-cyclopropyl-2H-tetrazol-2-yl)ethyl)-10-(2,2-difluoroethyl)-20-fluoro- |
| | 7-isobutyl-6,9-dimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 894 | (3S,7S,10R,13R)-13-benzyl-10-(2-ethoxyethyl)-20-fluoro-7-isobutyl-6,9-dimethyl-N-(2-(3- |
| | methylisoxazol-5-yl)ethyl)-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 895 | (3S,7S,10R,13R)-13-benzyl-10-(2-ethoxyethyl)-20-fluoro-7-isobutyl-N-(2-(3-methoxyisoxazol-5-yl)ethyl)- |
| | 6,9-dimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 896 | (3S,7S,10R,13R)-13-benzyl-N-(2-(5-cyclopropyl-2H-tetrazol-2-yl)ethyl)-10-(2-ethoxyethyl)-20-fluoro-7- |
| | isobutyl-6,9-dimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 897 | (3R,6R,9S,13S)-3-benzyl-9-isobutyl-16-methoxy-N-(2-(3-methoxyisoxazol-5-yl)ethyl)-7,10,18-trimethyl- |
| | 5,8,11,15-tetraoxo-6-((3-(trifluoromethyl)-1,2,4-oxadiazol-5-yl)methyl)-2,3,4,5,6,7,8,9,10,11,12,13,14,15- |
| | tetradecahydropyrido[3,4-p][1]oxa[4,7,10,14]tetraazacycloheptadecine-13-carboxamide |
| 898 | (3S,7S,10R,13R)-13-benzyl-7-isobutyl-N-(2-(3-methoxyisoxazol-5-yl)ethyl)-6,9,20-trimethyl-1,5,8,11- |
| | tetraoxo-10-((3-(trifluoromethyl)-1,2,4-oxadiazol-5-yl)methyl)-1,2,3,4,5,6,7,8,9,10,11,12,13,14- |
| | tetradecahydro-[1]oxa[4,7,10,14]tetraazacycloheptadecino[16,17-f]isoquinoline-3-carboxamide |
| 899 | (9S,13S,19aR,22S)-22-benzyl-5-fluoro-13-isobutyl-N-(2-(3-methoxyisoxazol-5-yl)ethyl)-12-methyl- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[16,17-f]quinoline-9-carboxamide |
| 900 | (3′S,7′S,13′R)-13′-benzyl-N-(2-(5-cyclopropyl-1,2,4-oxadiazol-3-yl)ethyl)-7′-isobutyl-6′,9′,20′-trimethyl- |
| | 1′,5′,8′,11′-tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[16,17-f]isoquinoline]-3′-carboxamide |
| 901 | (3′S,7′S,13′R)-13′-benzyl-7′-isobutyl-N-(2-(3-methoxyisoxazol-5-yl)ethyl)-6′,9′,20′-trimethyl-1′,5′,8′,11′- |
| | tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[16,17-f]isoquinoline]-3′-carboxamide |
| 902 | (3R,6R,9S,13S)-3-benzyl-N-(2-(5-cyclopropyl-2H-tetrazol-2-yl)ethyl)-9-isobutyl-16-methoxy-7,10,18- |
| | trimethyl-5,8,11,15-tetraoxo-6-((3-(trifluoromethyl)-1,2,4-oxadiazol-5-yl)methyl)- |
| | 2,3,4,5,6,7,8,9,10,11,12,13,14,15-tetradecahydropyrido[3,4- |
| | p][1]oxa[4,7,10,14]tetraazacycloheptadecine-13-carboxamide |
| 903 | (3R,6R,9S,13S)-N-(2-(2H-benzo[d][1,2,3]triazol-2-yl)ethyl)-3-benzyl-9-isobutyl-16-methoxy-7,10,18- |
| | trimethyl-5,8,11,15-tetraoxo-6-((3-(trifluoromethyl)-1,2,4-oxadiazol-5-yl)methyl)- |
| | 2,3,4,5,6,7,8,9,10,11,12,13,14,15-tetradecahydropyrido[3,4- |
| | p][1]oxa[4,7,10,14]tetraazacycloheptadecine-13-carboxamide |
| 904 | (3′S,7′S,13′R)-N-(2-(2H-benzo[d][1,2,3]triazol-2-yl)ethyl)-13′-benzyl-7′-isobutyl-6′,9′,20′-trimethyl- |
| | 1′,5′,8′,11′-tetraoxo-2′,3′,4′,5′,6′,7′,8′,9′,11′,12′,13′,14′-dodecahydro-1′H-spiro[cyclopropane-1,10′- |
| | [1]oxa[4,7,10,14]tetraazacycloheptadecino[16,17-f]isoquinoline]-3′-carboxamide |
| 905 | (3S,7S,10R,13R)-13-benzyl-N-(2-(5-cyclopropyl-1,2,4-oxadiazol-3-yl)ethyl)-10-((3-(5-fluoropyridin-2-yl)- |
| | 1,2,4-oxadiazol-5-yl)methyl)-7-isobutyl-6,9,20-trimethyl-1,5,8,11-tetraoxo- |
| | 1,2,3,4,5,6,7,8,9,10,11,12,13,14-tetradecahydro-[1]oxa[4,7,10,14]tetraazacycloheptadecino[16,17- |
| | flisoquinoline-3-carboxamide |
| 906 | (3S,7S,10R,13R)-13-benzyl-10-((3-(5-fluoropyridin-2-yl)-1,2,4-oxadiazol-5-yl)methyl)-7-isobutyl-N-(2-(3- |
| | methoxyisoxazol-5-yl)ethyl)-6,9,20-trimethyl-1,5,8,11-tetraoxo-1,2,3,4,5,6,7,8,9,10,11,12,13,14- |
| | tetradecahydro-[1]oxa[4,7,10,14]tetraazacycloheptadecino[16,17-f]isoquinoline-3-carboxamide |
| 907 | (3S,7S,10R,13R)-13-benzyl-N-(2-(5-cyclopropyl-2H-tetrazol-2-yl)ethyl)-10-((3-(5-fluoropyridin-2-yl)- |
| | 1,2,4-oxadiazol-5-yl)methyl)-7-isobutyl-6,9,20-trimethyl-1,5,8,11-tetraoxo- |
| | 1,2,3,4,5,6,7,8,9,10,11,12,13,14-tetradecahydro-[1]oxa[4,7,10,14]tetraazacycloheptadecino[16,17- |
| | flisoquinoline-3-carboxamide |
| 908 | (3S,7S,10R,13R)-N-(2-([1,2,4]triazolo[1,5-a]pyrimidin-2-yl)ethyl)-13-benzyl-20-fluoro-7-isobutyl-6,9- |
| | dimethyl-1,5,8,11-tetraoxo-10-((3-(trifluoromethyl)-1,2,4-oxadiazol-5-yl)methyl)- |
| | 1,2,3,4,5,6,7,8,9,10,11,12,13,14-tetradecahydro-[1]oxa[4,7,10,14]tetraazacycloheptadecino[16,17- |
| | f]quinoline-3-carboxamide |
| 909 | (3S,7S,10R,13R)-13-benzyl-7-isobutyl-N-(2-(3-methoxyisoxazol-5-yl)ethyl)-6,9,17-trimethyl-1,5,8,11- |
| | tetraoxo-10-((3-(trifluoromethyl)-1,2,4-oxadiazol-5-yl)methyl)-1,2,3,4,5,6,7,8,9,10,11,12,13,14- |
| | tetradecahydro-[1]oxa[4,7,10,14]tetraazacycloheptadecino[16,17-f]quinoline-3-carboxamide |
| 910 | (3R,6R,9S,13S)-3-benzyl-N-(2-(3-cyclopropylisoxazol-5-yl)ethyl)-6-(2-ethoxyethyl)-9-isobutyl-16- |
| | methoxy-7,10,18-trimethyl-5,8,11,15-tetraoxo-2,3,4,5,6,7,8,9,10,11,12,13,14,15- |
| | tetradecahydropyrido[3,4-p][1]oxa[4,7,10,14]tetraazacycloheptadecine-13-carboxamide |
| 911 | (3′S,7′S,13′R)-13′-benzyl-20′-fluoro-7′-isobutyl-N-(2-(3-methoxy-1,2,4-oxadiazol-5-yl)ethyl)-6′,9′- |
| | dimethyl-1′,5′,8′,11′-tetraoxo-2′,3′,4′,5′,6′,7′,8′,9′,11′,12′,13′,14′-dodecahydro-1′H-spiro[cyclopropane- |
| | 1,10′-[1]oxa[4,7,10,14]tetraazacycloheptadecino[16,17-f]quinoline]-3′-carboxamide |
| 912 | (9S,13S,19aR,22R)-22-benzyl-13-isobutyl-N-(2-(3-methoxyisoxazol-5-yl)ethyl)-12-methyl-7,11,14,20- |
| | tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[16,17-f]quinoline-9-carboxamide |
| 913 | (3S,7S,10R,13R)-13-benzyl-10-(2-ethoxyethyl)-7-isobutyl-N-(2-(3-methoxyisoxazol-5-yl)ethyl)-6,9,20- |
| | trimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 914 | (3S,7S,10R,13R)-13-benzyl-10-(2-ethoxyethyl)-7-isobutyl-N-(2-(3-methoxy-1,2,4-oxadiazol-5-yl)ethyl)- |
| | 6,9,20-trimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 915 | (3S,7S,10R,13R)-13-benzyl-N-(2-(5-cyclopropyl-2H-tetrazol-2-yl)ethyl)-10-(2-ethoxyethyl)-7-isobutyl- |
| | 6,9,20-trimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 916 | (3S,7S,10R,13R)-13-benzyl-20-fluoro-10-((3-(5-fluoropyridin-2-yl)-1,2,4-oxadiazol-5-yl)methyl)-7- |
| | isobutyl-N-(2-(3-methoxy-1,2,4-oxadiazol-5-yl)ethyl)-6,9-dimethyl-1,5,8,11-tetraoxo- |
| | 1,2,3,4,5,6,7,8,9,10,11,12,13,14-tetradecahydro-[1]oxa[4,7,10,14]tetraazacycloheptadecino[16,17- |
| | f]quinoline-3-carboxamide |
| 917 | (3S,7S,10R,13R)-13-benzyl-10-((3-(5-fluoropyridin-2-yl)-1,2,4-oxadiazol-5-yl)methyl)-7-isobutyl-N-(2-(3- |
| | methoxy-1,2,4-oxadiazol-5-yl)ethyl)-6,9,20-trimethyl-1,5,8,11-tetraoxo-1,2,3,4,5,6,7,8,9,10,11,12,13,14- |
| | tetradecahydro-[1]oxa[4,7,10,14]tetraazacycloheptadecino[16,17-f]isoquinoline-3-carboxamide |
| 918 | (3S,7S,10R,13R)-13-benzyl-7-isobutyl-N-(2-(3-methoxy-1,2,4-oxadiazol-5-yl)ethyl)-6,9,20-trimethyl- |
| | 1,5,8,11-tetraoxo-10-((3-(trifluoromethyl)-1,2,4-oxadiazol-5-yl)methyl)-1,2,3,4,5,6,7,8,9,10,11,12,13,14- |
| | tetradecahydro-[1]oxa[4,7,10,14]tetraazacycloheptadecino[16,17-f]isoquinoline-3-carboxamide |
| 919 | (3R,6R,9S,13S)-3-benzyl-9-isobutyl-16-methoxy-N-(2-(3-methoxy-1,2,4-oxadiazol-5-yl)ethyl)-7,10,18- |
| | trimethyl-5,8,11,15-tetraoxo-6-((3-(trifluoromethyl)-1,2,4-oxadiazol-5-yl)methyl)- |
| | 2,3,4,5,6,7,8,9,10,11,12,13,14,15-tetradecahydropyrido[3,4- |
| | p][1]oxa[4,7,10,14]tetraazacycloheptadecine-13-carboxamide |
| 920 | (9S,13S,19aR,22R)-22-benzyl-13-isobutyl-N-(2-(3-methoxy-1,2,4-oxadiazol-5-yl)ethyl)-2,12-dimethyl- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[16,17-f]quinoline-9-carboxamide |
| 921 | (3S,7S,10R,13R)-13-benzyl-N-(2-(5-cyclopropyl-2H-tetrazol-2-yl)ethyl)-10-((3-(difluoromethyl)-1,2,4- |
| | oxadiazol-5-yl)methyl)-7-isobutyl-6,9,20-trimethyl-1,5,8,11-tetraoxo-1,2,3,4,5,6,7,8,9,10,11,12,13,14- |
| | tetradecahydro-[1]oxa[4,7,10,14]tetraazacycloheptadecino[16,17-f]isoquinoline-3-carboxamide |
| 922 | (3′S,7′S,13′R)-13′-benzyl-3,3,20′-trifluoro-7′-isobutyl-N-(2-(3-methoxy-1,2,4-oxadiazol-5-yl)ethyl)-6′,9′- |
| | dimethyl-1′,5′,8′,11′-tetraoxo-2′,3′,4′,5′,6′,7′,8′,9′,11′,12′,13′,14′-dodecahydro-1′H-spiro[cyclobutane-1,10′- |
| | [1]oxa[4,7,10,14]tetraazacycloheptadecino[16,17-f]quinoline]-3′-carboxamide |
| 923 | (3S,7S,10R,13R)-13-benzyl-N-(2-(5-cyclopropyl-2H-tetrazol-2-yl)ethyl)-7-isobutyl-6,9,17-trimethyl- |
| | 1,5,8,11-tetraoxo-10-((3-(trifluoromethyl)-1,2,4-oxadiazol-5-yl)methyl)-1,2,3,4,5,6,7,8,9,10,11,12,13,14- |
| | tetradecahydro-[1]oxa[4,7,10,14]tetraazacycloheptadecino[16,17-f]quinoline-3-carboxamide |
| 924 | (3S,7S,10R,13R)-13-benzyl-N-(2-(5-cyclopropyl-2H-tetrazol-2-yl)ethyl)-20-fluoro-7-isobutyl-6,9- |
| | dimethyl-1,5,8,11-tetraoxo-10-((3-(trifluoromethyl)-1,2,4-oxadiazol-5-yl)methyl)- |
| | 1,2,3,4,5,6,7,8,9,10,11,12,13,14-tetradecahydro-[1]oxa[4,7,10,14]tetraazacycloheptadecino[16,17- |
| | f]quinoline-3-carboxamide |
| 925 | (3S,7S,10R,13R)-13-benzyl-20-fluoro-7-isobutyl-6,9-dimethyl-1,5,8,11-tetraoxo-N-(3- |
| | (trifluoromethoxy)propyl)-10-((3-(trifluoromethyl)-1,2,4-oxadiazol-5-yl)methyl)- |
| | 1,2,3,4,5,6,7,8,9,10,11,12,13,14-tetradecahydro-[1]oxa[4,7,10,14]tetraazacycloheptadecino[16,17- |
| | f]quinoline-3-carboxamide |
| 926 | (1r,3S,3′S,7′S,13′R)-13′-benzyl-20′-fluoro-7′-isobutyl-3-methoxy-N-(2-(3-methoxyisoxazol-5-yl)ethyl)- |
| | 6′,9′-dimethyl-1′,5′,8′,11′-tetraoxo-2′,3′,4′,5′,6′,7′,8′,9′,11′,12′,13′,14′-dodecahydro-1′H-spiro[cyclobutane- |
| | 1,10′-[1]oxa[4,7,10,14]tetraazacycloheptadecino[16,17-f]quinoline]-3′-carboxamide |
| 927 | (3S,7S,10R,13R)-13-benzyl-7-isobutyl-N-(2-(3-methoxy-1,2,4-oxadiazol-5-yl)ethyl)-6,9,17-trimethyl- |
| | 1,5,8,11-tetraoxo-10-((3-(trifluoromethyl)-1,2,4-oxadiazol-5-yl)methyl)-1,2,3,4,5,6,7,8,9,10,11,12,13,14- |
| | tetradecahydro-[1]oxa[4,7,10,14]tetraazacycloheptadecino[16,17-f]quinoline-3-carboxamide |
| 928 | (9S,13S,19aS,22R)-22-benzyl-N-(2-(5-cyclopropyl-2H-tetrazol-2-yl)ethyl)-13-isobutyl-2,12-dimethyl- |
| | 7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[16,17-f]quinoline-9-carboxamide |
| 929 | (3S,7S,10R,13R)-13-benzyl-10-(2-ethoxyethyl)-20-fluoro-7-isobutyl-N-(2-(3-methoxy-1,2,4-oxadiazol-5- |
| | yl)ethyl)-6,9-dimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 930 | (9S,13S,19aR,22R)-22-benzyl-5-fluoro-13-isobutyl-N-(2-(3-methoxy-1,2,4-oxadiazol-5-yl)ethyl)-12- |
| | methyl-7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[17,16-c]quinoline-9-carboxamide |
| 931 | (3S,7S,10R,13R)-13-benzyl-10-(2,2-difluoroethyl)-7-isobutyl-N-(2-(3-methoxyisoxazol-5-yl)ethyl)-6,9,20- |
| | trimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 932 | (3S,7S,10R,13R)-13-benzyl-10-(2,2-difluoroethyl)-7-isobutyl-N-(2-(3-methoxy-1,2,4-oxadiazol-5- |
| | yl)ethyl)-6,9,20-trimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 933 | (3S,7S,10R,13R)-13-benzyl-N-(2-(5-cyclopropyl-2H-tetrazol-2-yl)ethyl)-10-(2,2-difluoroethyl)-7-isobutyl- |
| | 6,9,20-trimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 934 | (3S,7S,10R,13R)-13-benzyl-20-fluoro-7-isobutyl-N-(2-(3-methoxy-1,2,4-oxadiazol-5-yl)ethyl)-6,9- |
| | dimethyl-1,5,8,11-tetraoxo-10-((3-(trifluoromethyl)-1,2,4-oxadiazol-5-yl)methyl)- |
| | 1,2,3,4,5,6,7,8,9,10,11,12,13,14-tetradecahydro-[1]oxa[4,7,10,14]tetraazacycloheptadecino[16,17- |
| | f]quinoline-3-carboxamide |
| 935 | (3S,7S,10R,13R)-13-benzyl-N-(2-(5-cyclopropyl-1,2,4-oxadiazol-3-yl)ethyl)-7-isobutyl-6,9,17-trimethyl- |
| | 1,5,8,11-tetraoxo-10-((3-(trifluoromethyl)-1,2,4-oxadiazol-5-yl)methyl)-1,2,3,4,5,6,7,8,9,10,11,12,13,14- |
| | tetradecahydro-[1]oxa[4,7,10,14]tetraazacycloheptadecino[16,17-f]quinoline-3-carboxamide |
| 936 | (3S,7S,10R,13R)-13-benzyl-N-(2-(3-cyclopropyl-1,2,4-oxadiazol-5-yl)ethyl)-7-isobutyl-6,9,17-trimethyl- |
| | 1,5,8,11-tetraoxo-10-((3-(trifluoromethyl)-1,2,4-oxadiazol-5-yl)methyl)-1,2,3,4,5,6,7,8,9,10,11,12,13,14- |
| | tetradecahydro-[1]oxa[4,7,10,14]tetraazacycloheptadecino[16,17-f]quinoline-3-carboxamide |
| 937 | (3S,7S,10R,13R)-N-(2-(2H-benzo[d][1,2,3]triazol-2-yl)ethyl)-13-benzyl-20-fluoro-7-isobutyl-6,9-dimethyl- |
| | 1,5,8,11-tetraoxo-10-((3-(trifluoromethyl)-1,2,4-oxadiazol-5-yl)methyl)-1,2,3,4,5,6,7,8,9,10,11,12,13,14- |
| | tetradecahydro-[1]oxa[4,7,10,14]tetraazacycloheptadecino[16,17-f]quinoline-3-carboxamide |
| 938 | (3S,7S,10R,13R)-13-benzyl-7-isobutyl-N-(2-(3-methoxy-1,2,4-oxadiazol-5-yl)ethyl)-6,9,20-trimethyl- |
| | 1,5,8,11-tetraoxo-10-(2,2,2-trifluoroethyl)-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 939 | (3S,7S,10S,13R)-13-benzyl-7-isobutyl-N-(2-(3-methoxy-1,2,4-oxadiazol-5-yl)ethyl)-6,9,20-trimethyl- |
| | 1,5,8,11-tetraoxo-10-(2,2,2-trifluoroethyl)-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 940 | (3S,7S,10S,13R)-13-benzyl-20-fluoro-7-isobutyl-N-(2-(3-methoxy-1,2,4-oxadiazol-5-yl)ethyl)-6,9- |
| | dimethyl-1,5,8,11-tetraoxo-10-(2,2,2-trifluoroethyl)-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 941 | (3S,7S,10R,13R)-13-benzyl-20-fluoro-7-isobutyl-N-(2-(3-methoxy-1,2,4-oxadiazol-5-yl)ethyl)-6,9- |
| | dimethyl-1,5,8,11-tetraoxo-10-(2,2,2-trifluoroethyl)-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 942 | (3R,6R,9S,13S)-3-benzyl-9-isobutyl-16-methoxy-N-(2-(3-methoxy-1,2,4-oxadiazol-5-yl)ethyl)-7,10,18- |
| | trimethyl-5,8,11,15-tetraoxo-6-(2,2,2-trifluoroethyl)-2,3,4,5,6,7,8,9,10,11,12,13,14,15- |
| | tetradecahydropyrido[3,4-p][1]oxa[4,7,10,14]tetraazacycloheptadecine-13-carboxamide |
| 943 | (3R,6R,9S,13S)-3-benzyl-N-((R)-2-(3-cyclopropyl-1,2,4-oxadiazol-5-yl)-2-hydroxyethyl)-9-isobutyl-16- |
| | methoxy-7,10,18-trimethyl-5,8,11,15-tetraoxo-6-((3-(trifluoromethyl)-1,2,4-oxadiazol-5-yl)methyl)- |
| | 2,3,4,5,6,7,8,9,10,11,12,13,14,15-tetradecahydropyrido[3,4- |
| | p][1]oxa[4,7,10,14]tetraazacycloheptadecine-13-carboxamide |
| 944 | (3S,7S,10R,13R)-13-benzyl-N-(3-(difluoromethoxy)propyl)-20-fluoro-7-isobutyl-6,9-dimethyl-1,5,8,11- |
| | tetraoxo-10-((3-(trifluoromethyl)-1,2,4-oxadiazol-5-yl)methyl)-1,2,3,4,5,6,7,8,9,10,11,12,13,14- |
| | tetradecahydro-[1]oxa[4,7,10,14]tetraazacycloheptadecino[16,17-f]quinoline-3-carboxamide |
| 945 | (3R,6R,9S,13S)-3-benzyl-N-(2-(4-fluoro-3-methoxyisoxazol-5-yl)ethyl)-9-isobutyl-16-methoxy-7,10,18- |
| | trimethyl-5,8,11,15-tetraoxo-6-((3-(trifluoromethyl)-1,2,4-oxadiazol-5-yl)methyl)- |
| | 2,3,4,5,6,7,8,9,10,11,12,13,14,15-tetradecahydropyrido[3,4- |
| | p][1]oxa[4,7,10,14]tetraazacycloheptadecine-13-carboxamide |
| 946 | (3S,7S,10R,13R)-13-benzyl-10-(2,2-difluoroethyl)-20-fluoro-7-isobutyl-6,9-dimethyl-N-(2-(3- |
| | methylisoxazol-5-yl)ethyl)-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 947 | (3S,7S,10R,13R)-13-benzyl-10-(2,2-difluoroethyl)-20-fluoro-7-isobutyl-N-(2-(3-methoxy-1,2,4-oxadiazol- |
| | 5-yl)ethyl)-6,9-dimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 948 | (3R,6R,9S,13S)-3-benzyl-9-isobutyl-16-methoxy-N-(2-(3-methoxyisoxazol-5-yl)ethyl)-7,10-dimethyl- |
| | 5,8,11,15-tetraoxo-6-((3-(trifluoromethyl)-1,2,4-oxadiazol-5-yl)methyl)-2,3,4,5,6,7,8,9,10,11,12,13,14,15- |
| | tetradecahydropyrido[3,4-p][1]oxa[4,7,10,14]tetraazacycloheptadecine-13-carboxamide |
| 949 | (3R,6R,9S,13S)-3-benzyl-9-isobutyl-16-methoxy-N-(2-(3-methoxy-1,2,4-oxadiazol-5-yl)ethyl)-7,10- |
| | dimethyl-5,8,11,15-tetraoxo-6-((3-(trifluoromethyl)-1,2,4-oxadiazol-5-yl)methyl)- |
| | 2,3,4,5,6,7,8,9,10,11,12,13,14,15-tetradecahydropyrido[3,4- |
| | p][1]oxa[4,7,10,14]tetraazacycloheptadecine-13-carboxamide |
| 950 | (3S,7S,10R,13R)-13-benzyl-N-(2-(5-cyclopropyl-1,2,4-oxadiazol-3-yl)ethyl)-7-isobutyl-6,9,20-trimethyl- |
| | 1,5,8,11-tetraoxo-10-(2,2,2-trifluoroethyl)-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 951 | (3S,7S,10R,13R)-13-benzyl-N-(2-(3-cyclopropyl-1,2,4-oxadiazol-5-yl)ethyl)-7-isobutyl-6,9,20-trimethyl- |
| | 1,5,8,11-tetraoxo-10-(2,2,2-trifluoroethyl)-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 952 | (3′S,7′S,13′R)-13′-benzyl-N-(2-(3-cyclopropyl-1,2,4-oxadiazol-5-yl)ethyl)-3,3-difluoro-7′-isobutyl-6′,9′,20′- |
| | trimethyl-1′,5′,8′,11′-tetraoxo-2′,3′,4′,5′,6′,7′,8′,9′,11′,12′,13′,14′-dodecahydro-1′H-spiro[cyclobutane-1,10′- |
| | [1]oxa[4,7,10,14]tetraazacycloheptadecino[16,17-f]isoquinoline]-3′-carboxamide |
| 953 | (3′S,7′S,13′R)-13′-benzyl-3,3-difluoro-7′-isobutyl-N-(2-(3-methoxyisoxazol-5-yl)ethyl)-6′,9′,20′-trimethyl- |
| | 1′,5′,8′,11′-tetraoxo-2′,3′,4′,5′,6′,7′,8′,9′,11′,12′,13′,14′-dodecahydro-1′H-spiro[cyclobutane-1,10′- |
| | [1]oxa[4,7,10,14]tetraazacycloheptadecino[16,17-f]isoquinoline]-3′-carboxamide |
| 954 | (3′S,7′S,13′R)-13′-benzyl-3,3-difluoro-7′-isobutyl-N-(2-(3-methoxy-1,2,4-oxadiazol-5-yl)ethyl)-6′,9′,20′- |
| | trimethyl-1′,5′,8′,11′-tetraoxo-2′,3′,4′,5′,6′,7′,8′,9′,11′,12′,13′,14′-dodecahydro-1′H-spiro[cyclobutane-1,10′- |
| | [1]oxa[4,7,10,14]tetraazacycloheptadecino[16,17-f]isoquinoline]-3′-carboxamide |
| 955 | (3′S,7′S,13′R)-13′-benzyl-N-(2-(5-cyclopropyl-2H-tetrazol-2-yl)ethyl)-3,3-difluoro-7′-isobutyl-6′,9′,20′- |
| | trimethyl-1′,5′,8′,11′-tetraoxo-2′,3′,4′,5′,6′,7′,8′,9′,11′,12′,13′,14′-dodecahydro-1′H-spiro[cyclobutane-1,10′- |
| | [1]oxa[4,7,10,14]tetraazacycloheptadecino[16,17-f]isoquinoline]-3′-carboxamide |
| 956 | (3S,7S,10R,13R)-13-benzyl-N-(2-(3-cyclopropylisoxazol-5-yl)ethyl)-7-isobutyl-6,9,20-trimethyl-1,5,8,11- |
| | tetraoxo-10-(2,2,2-trifluoroethyl)-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 957 | (3S,7S,10R,13R)-13-benzyl-7-isobutyl-N-(2-(3-methoxyisoxazol-5-yl)ethyl)-6,9,20-trimethyl-1,5,8,11- |
| | tetraoxo-10-(2,2,2-trifluoroethyl)-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 958 | (3S,7S,10R,13R)-13-benzyl-N-(2-(5-cyclopropylisoxazol-3-yl)ethyl)-7-isobutyl-6,9,20-trimethyl-1,5,8,11- |
| | tetraoxo-10-(2,2,2-trifluoroethyl)-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 959 | (3S,7S,10R,13R)-13-benzyl-N-(2-(5-cyclopropyl-2H-tetrazol-2-yl)ethyl)-7-isobutyl-6,9,20-trimethyl- |
| | 1,5,8,11-tetraoxo-10-(2,2,2-trifluoroethyl)-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 960 | (3S,7S,10R,13R)-13-benzyl-N-(2-(4-fluoro-3-methoxyisoxazol-5-yl)ethyl)-7-isobutyl-6,9,20-trimethyl- |
| | 1,5,8,11-tetraoxo-10-(2,2,2-trifluoroethyl)-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 961 | (3R,6R,9S,13S)-3-benzyl-9-isobutyl-16-methoxy-N-(2-(1-(2-methoxyethyl)-1H-pyrazol-4-yl)ethyl)- |
| | 7,10,18-trimethyl-5,8,11,15-tetraoxo-6-((3-(trifluoromethyl)-1,2,4-oxadiazol-5-yl)methyl)- |
| | 2,3,4,5,6,7,8,9,10,11,12,13,14,15-tetradecahydropyrido[3,4- |
| | p][1]oxa[4,7,10,14]tetraazacycloheptadecine-13-carboxamide |
| 962 | (3S,7S,10R,13R)-13-benzyl-20-fluoro-7-isobutyl-N-(2-(1-(2-methoxyethyl)-1H-pyrazol-4-yl)ethyl)-6,9- |
| | dimethyl-1,5,8,11-tetraoxo-10-((3-(trifluoromethyl)-1,2,4-oxadiazol-5-yl)methyl)- |
| | 1,2,3,4,5,6,7,8,9,10,11,12,13,14-tetradecahydro-[1]oxa[4,7,10,14]tetraazacycloheptadecino[16,17- |
| | f]quinoline-3-carboxamide |
| 963 | (3′S,7′S,13′R)-13′-benzyl-20′-fluoro-N-(2-(4-fluoro-3-methoxyisoxazol-5-yl)ethyl)-7′-isobutyl-6′,9′- |
| | dimethyl-1′,5′,8′,11′-tetraoxo-2′,3′,4′,5′,6′,7′,8′,9′,11′,12′,13′,14′-dodecahydro-1′H-spiro[cyclopropane- |
| | 1,10′-[1]oxa[4,7,10,14]tetraazacycloheptadecino[16,17-f]quinoline]-3′-carboxamide |
| 964 | (3S,7S,10R,13R)-13-benzyl-N-(2-(3-cyclopropyl-1,2,4-oxadiazol-5-yl)ethyl)-20-fluoro-7-isobutyl-6,9- |
| | dimethyl-1,5,8,11-tetraoxo-10-((3-(trifluoromethyl)-1,2,4-oxadiazol-5-yl)methyl)- |
| | 1,2,3,4,5,6,7,8,9,10,11,12,13,14-tetradecahydro-[1]oxa[4,7,10,14]tetraazacycloheptadecino[16,17- |
| | f]quinoline-3-carboxamide |
| 965 | (3S,7S,10R,13R)-13-benzyl-20-fluoro-7-isobutyl-6,9-dimethyl-N-(2-(3-methylisoxazol-5-yl)ethyl)- |
| | 1,5,8,11-tetraoxo-10-((3-(trifluoromethyl)-1,2,4-oxadiazol-5-yl)methyl)-1,2,3,4,5,6,7,8,9,10,11,12,13,14- |
| | tetradecahydro-[1]oxa[4,7,10,14]tetraazacycloheptadecino[16,17-f]quinoline-3-carboxamide |
| 966 | (3S,7S,10R,13R)-13-benzyl-N-((R)-2-(3-cyclopropyl-1,2,4-oxadiazol-5-yl)-2-hydroxyethyl)-20-fluoro-7- |
| | isobutyl-6,9-dimethyl-1,5,8,11-tetraoxo-10-((3-(trifluoromethyl)-1,2,4-oxadiazol-5-yl)methyl)- |
| | 1,2,3,4,5,6,7,8,9,10,11,12,13,14-tetradecahydro-[1]oxa[4,7,10,14]tetraazacycloheptadecino[16,17- |
| | f]quinoline-3-carboxamide |
| 967 | (3S,7S,10R,13R)-13-benzyl-10-(2,2-difluoroethyl)-20-fluoro-N-(2-(4-fluoro-3-methoxyisoxazol-5- |
| | yl)ethyl)-7-isobutyl-6,9-dimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 968 | (3R,6R,9S,13S)-3-benzyl-6-(2,2-difluoroethyl)-N-(2-(4-fluoro-3-methoxyisoxazol-5-yl)ethyl)-9-isobutyl- |
| | 16-methoxy-7,10,18-trimethyl-5,8,11,15-tetraoxo-2,3,4,5,6,7,8,9,10,11,12,13,14,15- |
| | tetradecahydropyrido[3,4-p][1]oxa[4,7,10,14]tetraazacycloheptadecine-13-carboxamide |
| 969 | (3′S,7′S,13′R)-13′-benzyl-3,3,20′-trifluoro-N-(2-(4-fluoro-3-methoxyisoxazol-5-yl)ethyl)-7′-isobutyl-6′,9′- |
| | dimethyl-1′,5′,8′,11′-tetraoxo-2′,3′,4′,5′,6′,7′,8′,9′,11′,12′,13′,14′-dodecahydro-1′H-spiro[cyclobutane-1,10′- |
| | [1]oxa[4,7,10,14]tetraazacycloheptadecino[16,17-f]quinoline]-3′-carboxamide |
| 970 | (3S,7S,10R,13R)-13-benzyl-10-(2,2-difluoroethyl)-N-(2-(4-fluoro-3-methoxyisoxazol-5-yl)ethyl)-7- |
| | isobutyl-6,9,20-trimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 971 | (3S,7S,10R,13R)-13-benzyl-10-((5-(tert-butyl)-1,2,4-oxadiazol-3-yl)methyl)-20-fluoro-7-isobutyl-N-(2-(3- |
| | methoxy-1,2,4-oxadiazol-5-yl)ethyl)-6,9-dimethyl-1,5,8,11-tetraoxo-1,2,3,4,5,6,7,8,9,10,11,12,13,14- |
| | tetradecahydro-[1]oxa[4,7,10,14]tetraazacycloheptadecino[16,17-f]quinoline-3-carboxamide |
| 972 | (3S,7S,10R,13R)-13-benzyl-10-((5-(tert-butyl)-1,2,4-oxadiazol-3-yl)methyl)-20-fluoro-7-isobutyl-N-(2-(3- |
| | methoxyisoxazol-5-yl)ethyl)-6,9-dimethyl-1,5,8,11-tetraoxo-1,2,3,4,5,6,7,8,9,10,11,12,13,14- |
| | tetradecahydro-[1]oxa[4,7,10,14]tetraazacycloheptadecino[16,17-f]quinoline-3-carboxamide |
| 973 | (9S,13S,19aR,22R)-22-benzyl-5-fluoro-N-(2-(4-fluoro-3-methoxyisoxazol-5-yl)ethyl)-13-isobutyl-12- |
| | methyl-7,11,14,20-tetraoxo-7,8,9,10,11,12,13,14,17,18,19,19a,20,21,22,23-hexadecahydro-16H- |
| | pyrido[2′,1′:6,7][1]oxa[4,7,10,14]tetraazacycloheptadecino[16,17-f]quinoline-9-carboxamide |
| 974 | (8R,11RS,14S,18S)-8-benzyl-14-isobutyl-N-(2-(3-methoxyisoxazol-5-yl)ethyl)-2,12,15-trimethyl- |
| | 10,13,16,20-tetraoxo-11-(2,2,2-trifluoroethyl)-7,8,9,10,11,12,13,14,15,16,17,18,19,20- |
| | tetradecahydrooxazolo[4′,5′:5,6]benzo[1,2-p][1]oxa[4,7,10,14]tetraazacycloheptadecine-18- |
| | carboxamide |
| 975 | (3S,7S,10R,13R)-13-benzyl-7-isobutyl-N-(2-(3-methoxy-1,2,4-oxadiazol-5-yl)ethyl)-6,9,17-trimethyl- |
| | 1,5,8,11-tetraoxo-10-(2,2,2-trifluoroethyl)-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 976 | (3S,7S,10R,13R)-13-benzyl-N-(2-(3-cyclopropyl-1,2,4-oxadiazol-5-yl)ethyl)-10-(2-ethoxyethyl)-20- |
| | fluoro-7-isobutyl-6,9-dimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 977 | (3R,6R,9S,13S)-3-benzyl-9-isobutyl-16-methoxy-N-(2-(3-methoxyisoxazol-5-yl)ethyl)-7,10,18-trimethyl- |
| | 6-(2-((5-methylpyrazin-2-yl)oxy)ethyl)-5,8,11,15-tetraoxo-2,3,4,5,6,7,8,9,10,11,12,13,14,15- |
| | tetradecahydropyrido[3,4-p][1]oxa[4,7,10,14]tetraazacycloheptadecine-13-carboxamide |
| 978 | (3R,6R,9S,13S)-3-benzyl-9-isobutyl-16-methoxy-N-(2-(3-methoxyisoxazol-5-yl)ethyl)-6-(2-((2- |
| | methoxypyridin-4-yl)oxy)ethyl)-7,10,18-trimethyl-5,8,11,15-tetraoxo-2,3,4,5,6,7,8,9,10,11,12,13,14,15- |
| | tetradecahydropyrido[3,4-p][1]oxa[4,7,10,14]tetraazacycloheptadecine-13-carboxamide |
| 979 | (3R,6R,9S,13S)-3-benzyl-6-(cyclopropylmethyl)-9-isobutyl-16-methoxy-N-(2-(3-methoxyisoxazol-5- |
| | yl)ethyl)-7,10,18-trimethyl-5,8,11,15-tetraoxo-2,3,4,5,6,7,8,9,10,11,12,13,14,15- |
| | tetradecahydropyrido[3,4-p][1]oxa[4,7,10,14]tetraazacycloheptadecine-13-carboxamide |
| 980 | (3R,6R,9S,13S)-3-benzyl-6-(cyclobutylmethyl)-9-isobutyl-16-methoxy-N-(2-(3-methoxyisoxazol-5- |
| | yl)ethyl)-7,10,18-trimethyl-5,8,11,15-tetraoxo-2,3,4,5,6,7,8,9,10,11,12,13,14,15- |
| | tetradecahydropyrido[3,4-p][1]oxa[4,7,10,14]tetraazacycloheptadecine-13-carboxamide |
| 981 | (3S,7S,10R,13R)-13-benzyl-20-fluoro-7-isobutyl-3-((2-(3-methoxyisoxazol-5-yl)ethyl)carbamoyl)-6,9- |
| | dimethyl-1,5,8,11-tetraoxo-10-((3-(trifluoromethyl)-1,2,4-oxadiazol-5-yl)methyl)- |
| | 1,2,3,4,5,6,7,8,9,10,11,12,13,14-tetradecahydro-[1]oxa[4,7,10,14]tetraazacycloheptadecino[17,16- |
| | c]quinoline 17-oxide |
| 982 | (3S,7S,10R,13R)-13-benzyl-7-isobutyl-3-((2-(3-methoxyisoxazol-5-yl)ethyl)carbamoyl)-6,9,17-trimethyl- |
| | 1,5,8,11-tetraoxo-10-((3-(trifluoromethyl)-1,2,4-oxadiazol-5-yl)methyl)-1,2,3,4,5,6,7,8,9,10,11,12,13,14- |
| | tetradecahydro-[1]oxa[4,7,10,14]tetraazacycloheptadecino[16,17-f]quinoline 18-oxide |
| 983 | (3S,7S,10R,13R)-13-benzyl-20-fluoro-7-isobutyl-3-((2-(3-methoxy-1,2,4-oxadiazol-5- |
| | yl)ethyl)carbamoyl)-6,9-dimethyl-1,5,8,11-tetraoxo-10-((3-(trifluoromethyl)-1,2,4-oxadiazol-5-yl)methyl)- |
| | 1,2,3,4,5,6,7,8,9,10,11,12,13,14-tetradecahydro-[1]oxa[4,7,10,14]tetraazacycloheptadecino[16,17- |
| | f]quinoline 18-oxide |

TABLE 3
Structures of compounds of Example 1 to 764

| | |
|---|---|
| 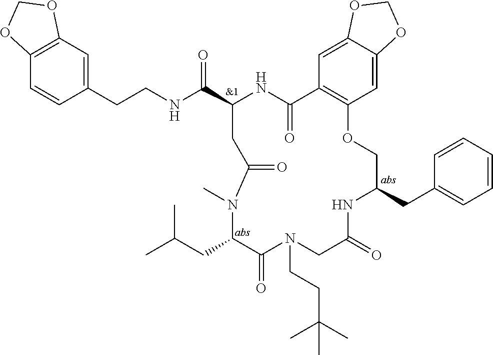 [SMILES: CC(C)C[C@H]1C(=O)N(CCC(C)(C)C)CC(=O)N[C@H](Cc2ccccc2)COc2cc3c(cc2C(=O)N[C@H](C(=O)NCCc2ccc4c(c2)OCO4)CC(=O)N1C)OCO3] | 1 |
| 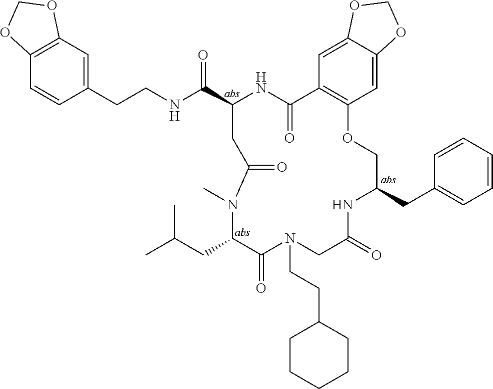 [SMILES: CC(C)C[C@H]1C(=O)N(CCC2CCCCC2)CC(=O)N[C@H](Cc2ccccc2)COc2cc3c(cc2C(=O)N[C@H](C(=O)NCCc2ccc4c(c2)OCO4)CC(=O)N1C)OCO3] | 2 |
| 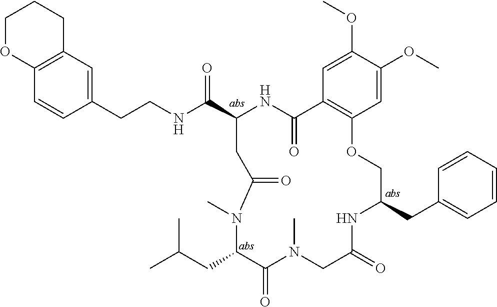 [SMILES: COc1cc2c(cc1OC)C(=O)N[C@H](C(=O)NCCc1ccc3c(c1)CCCO3)CC(=O)N(C)[C@@H](CC(C)C)C(=O)N(C)CC(=O)N[C@H](Cc1ccccc1)CO2] | 3 |
| 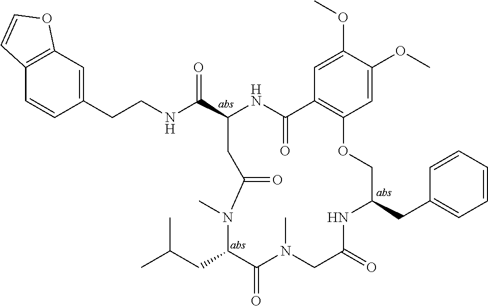 [SMILES: COc1cc2c(cc1OC)C(=O)N[C@H](C(=O)NCCc1ccc3ccoc3c1)CC(=O)N(C)[C@@H](CC(C)C)C(=O)N(C)CC(=O)N[C@H](Cc1ccccc1)CO2] | 4 |
| 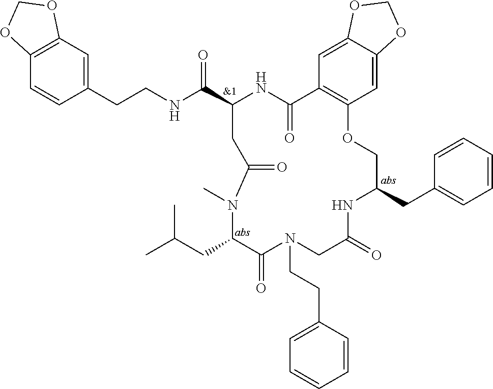 [SMILES: CC(C)C[C@H]1C(=O)N(CCc2ccccc2)CC(=O)N[C@H](Cc2ccccc2)COc2cc3c(cc2C(=O)N[C@H](C(=O)NCCc2ccc4c(c2)OCO4)CC(=O)N1C)OCO3] | 5 |
| 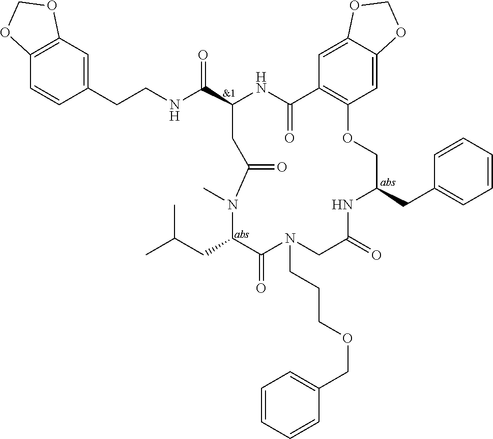 [SMILES: CC(C)C[C@H]1C(=O)N(CCCOCc2ccccc2)CC(=O)N[C@H](Cc2ccccc2)COc2cc3c(cc2C(=O)N[C@H](C(=O)NCCc2ccc4c(c2)OCO4)CC(=O)N1C)OCO3] | 6 |
| 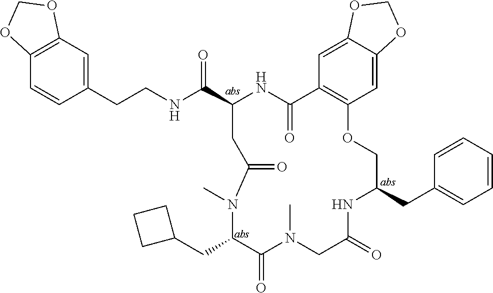 [SMILES: CN1CC(=O)N[C@H](Cc2ccccc2)COc2cc3c(cc2C(=O)N[C@H](C(=O)NCCc2ccc4c(c2)OCO4)CC(=O)N(C)[C@@H](CC2CCC2)C1=O)OCO3] | 7 |
| 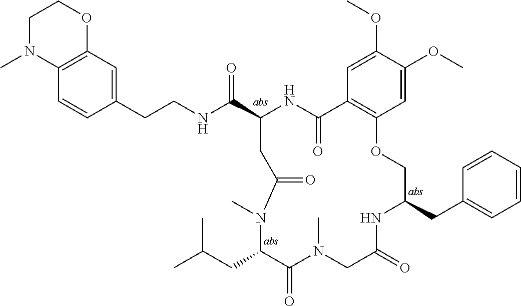 [SMILES: COc1cc2c(cc1OC)C(=O)N[C@H](C(=O)NCCc1ccc3c(c1)OCCN3C)CC(=O)N(C)[C@@H](CC(C)C)C(=O)N(C)CC(=O)N[C@H](Cc1ccccc1)CO2] | 8 |
| 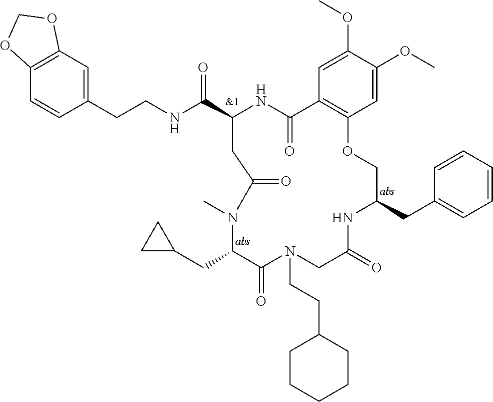 [SMILES: COc1cc2c(cc1OC)C(=O)N[C@H](C(=O)NCCc1ccc3c(c1)OCO3)CC(=O)N(C)[C@@H](CC1CC1)C(=O)N(CCC1CCCCC1)CC(=O)N[C@H](Cc1ccccc1)CO2] | 9 |
| 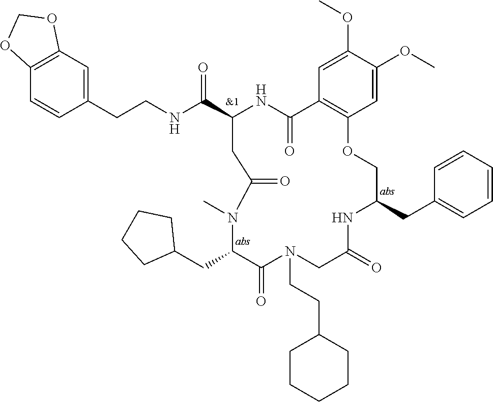 [SMILES: COc1cc2c(cc1OC)C(=O)N[C@H](C(=O)NCCc1ccc3c(c1)OCO3)CC(=O)N(C)[C@@H](CC1CCCC1)C(=O)N(CCC1CCCCC1)CC(=O)N[C@H](Cc1ccccc1)CO2] | 10 |
| 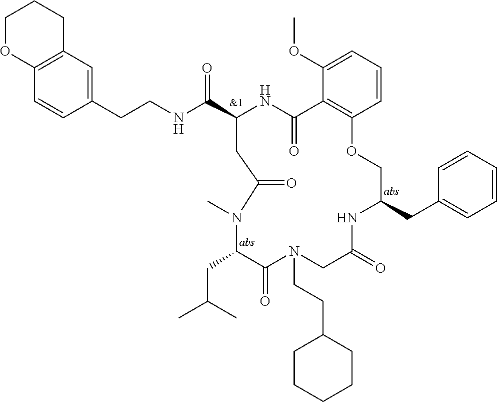 [SMILES: COc1cccc2c1C(=O)N[C@H](C(=O)NCCc1ccc3c(c1)CCCO3)CC(=O)N(C)[C@@H](CC(C)C)C(=O)N(CCC1CCCCC1)CC(=O)N[C@H](Cc1ccccc1)CO2] | 11 |
| 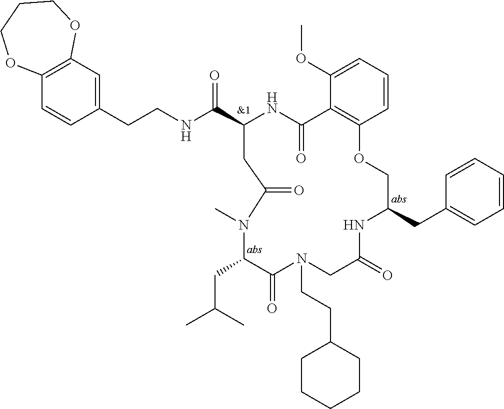 [SMILES: COc1cccc2c1C(=O)N[C@H](C(=O)NCCc1ccc3c(c1)OCCCO3)CC(=O)N(C)[C@@H](CC(C)C)C(=O)N(CCC1CCCCC1)CC(=O)N[C@H](Cc1ccccc1)CO2] | 12 |
| 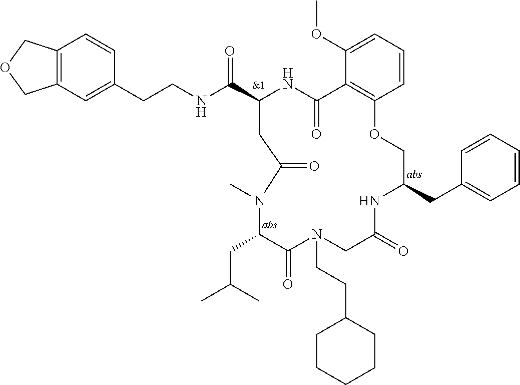 [SMILES: COc1cccc2c1C(=O)N[C@H](C(=O)NCCc1ccc3c(c1)COC3)CC(=O)N(C)[C@@H](CC(C)C)C(=O)N(CCC1CCCCC1)CC(=O)N[C@H](Cc1ccccc1)CO2] | 13 |
| 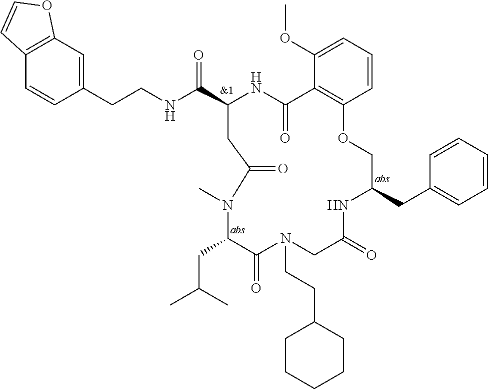 [SMILES: COc1cccc2c1C(=O)N[C@H](C(=O)NCCc1ccc3ccoc3c1)CC(=O)N(C)[C@@H](CC(C)C)C(=O)N(CCC1CCCCC1)CC(=O)N[C@H](Cc1ccccc1)CO2] | 14 |
| 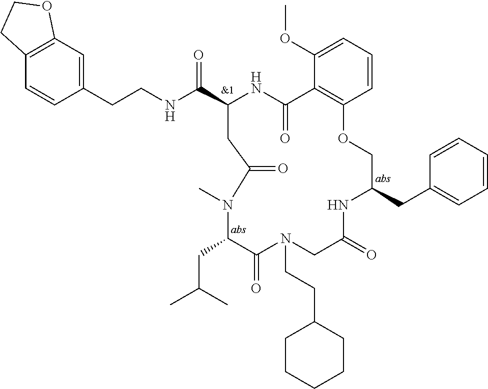 [SMILES: COc1cccc2c1C(=O)N[C@H](C(=O)NCCc1ccc3c(c1)OCC3)CC(=O)N(C)[C@@H](CC(C)C)C(=O)N(CCC1CCCCC1)CC(=O)N[C@H](Cc1ccccc1)CO2] | 15 |
| 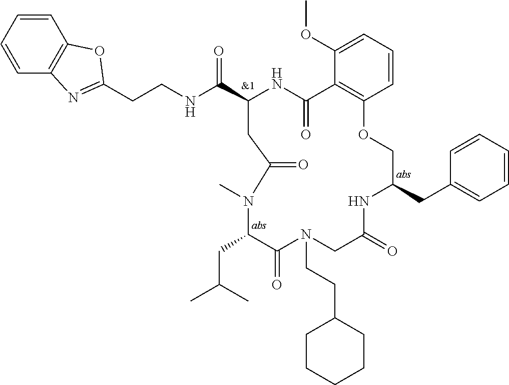 [SMILES: COc1cccc2c1C(=O)N[C@H](C(=O)NCCc1nc3ccccc3o1)CC(=O)N(C)[C@@H](CC(C)C)C(=O)N(CCC1CCCCC1)CC(=O)N[C@H](Cc1ccccc1)CO2] | 16 |
| 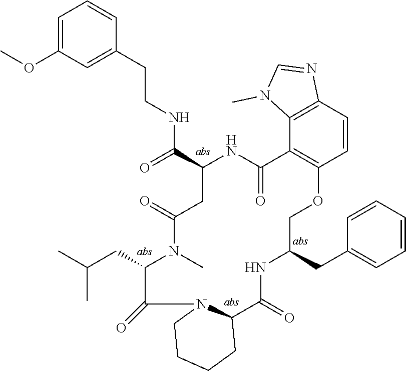 [SMILES: COc1cccc(CCNC(=O)[C@@H]2CC(=O)N(C)[C@@H](CC(C)C)C(=O)N3CCCC[C@@H]3C(=O)N[C@H](Cc3ccccc3)COc3ccc4ncn(C)c4c3C(=O)N2)c1] | 17 |
| 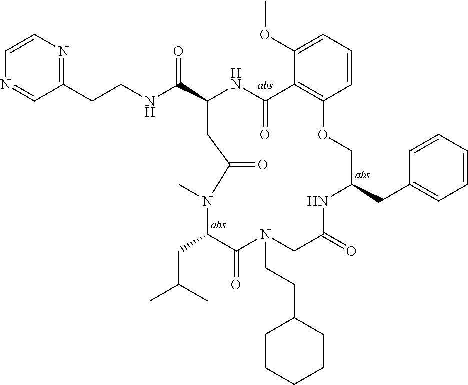 [SMILES: COc1cccc2c1C(=O)N[C@H](C(=O)NCCc1cnccn1)CC(=O)N(C)[C@@H](CC(C)C)C(=O)N(CCC1CCCCC1)CC(=O)N[C@H](Cc1ccccc1)CO2] | 18 |
| 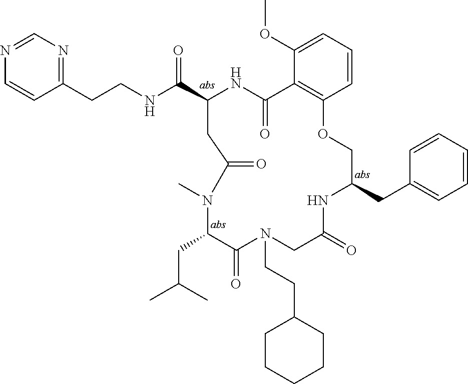 [SMILES: COc1cccc2c1C(=O)N[C@H](C(=O)NCCc1ccncn1)CC(=O)N(C)[C@@H](CC(C)C)C(=O)N(CCC1CCCCC1)CC(=O)N[C@H](Cc1ccccc1)CO2] | 19 |
| 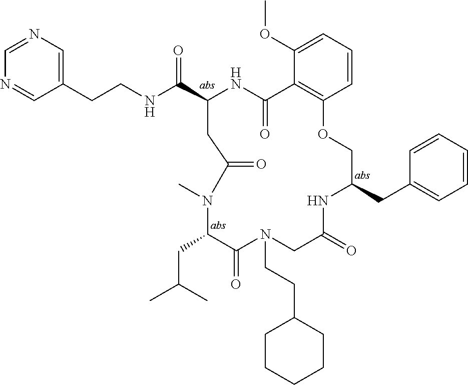 [SMILES: COc1cccc2c1C(=O)N[C@H](C(=O)NCCc1cncnc1)CC(=O)N(C)[C@@H](CC(C)C)C(=O)N(CCC1CCCCC1)CC(=O)N[C@H](Cc1ccccc1)CO2] | 20 |
| 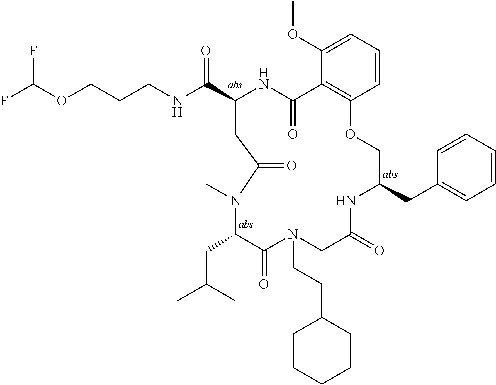 [SMILES: COc1cccc2c1C(=O)N[C@H](C(=O)NCCCOC(F)F)CC(=O)N(C)[C@@H](CC(C)C)C(=O)N(CCC1CCCCC1)CC(=O)N[C@H](Cc1ccccc1)CO2] | 21 |
| 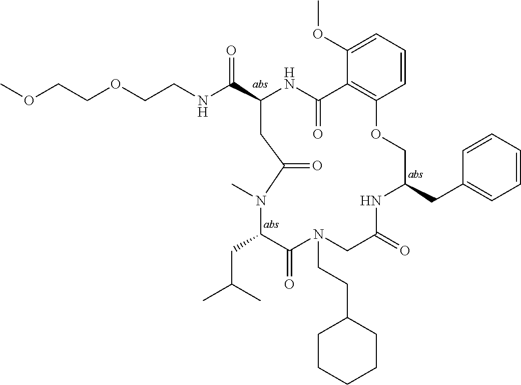 [SMILES: COCCOCCNC(=O)[C@@H]1CC(=O)N(C)[C@@H](CC(C)C)C(=O)N(CCC2CCCCC2)CC(=O)N[C@H](Cc2ccccc2)COc2cccc(OC)c2C(=O)N1] | 22 |
| 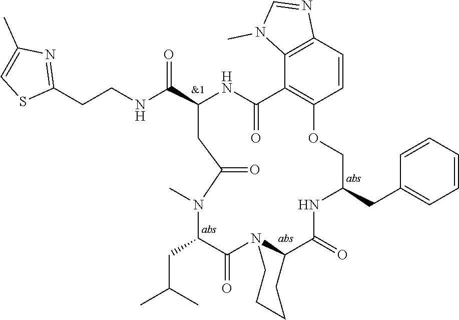 | 23 |
| 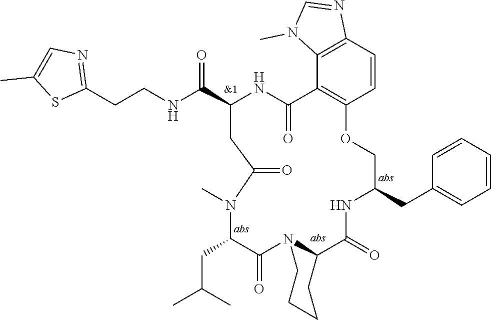 | 24 |
| 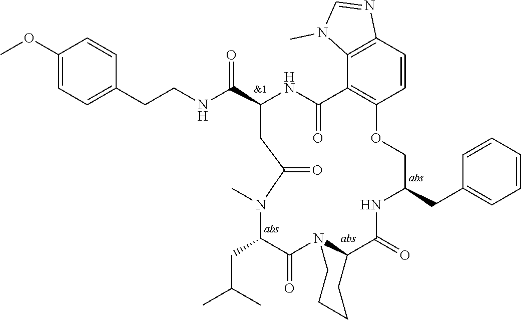 | 25 |
| 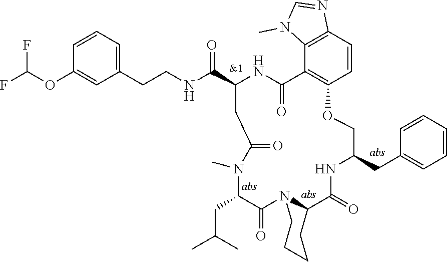 | 26 |
| 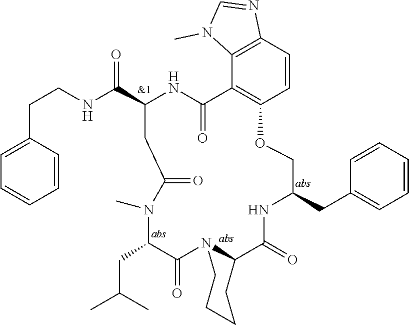 | 27 |
| 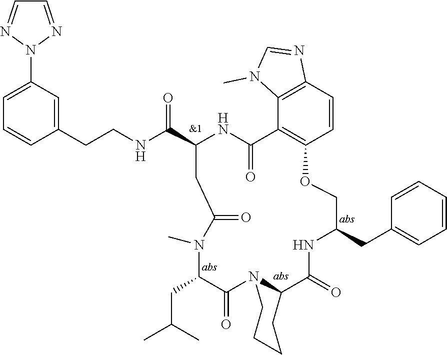 | 28 |
| 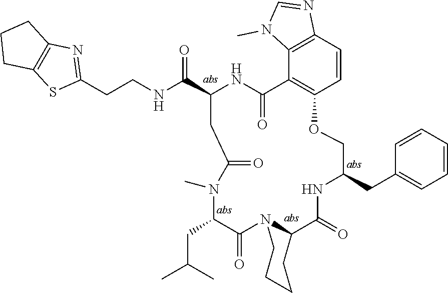 | 29 |
| 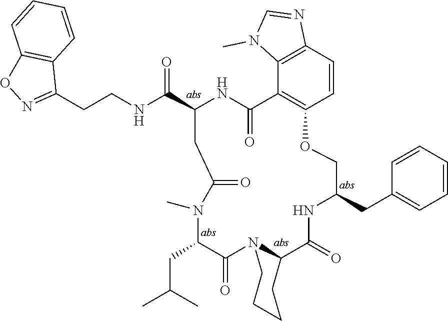 | 30 |
| 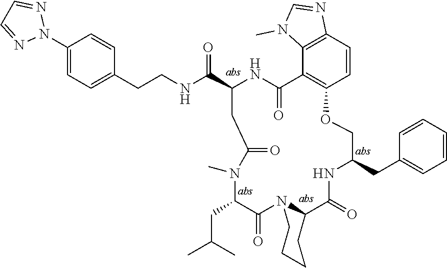 | 31 |
| 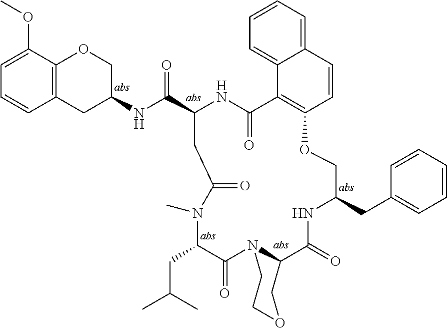 | 32 |
| 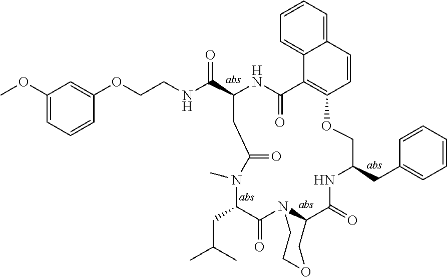 | 33 |
| 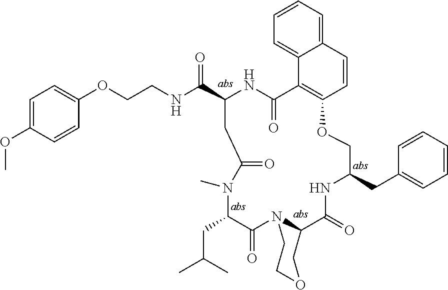 | 34 |
| 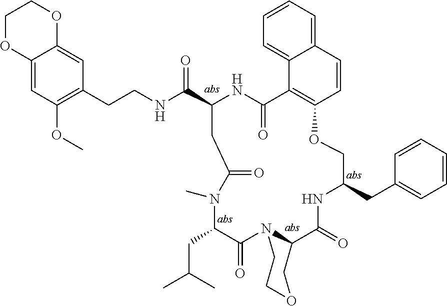 | 35 |
| 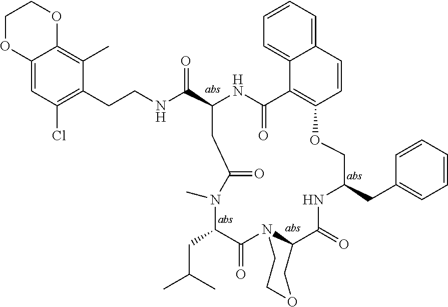 | 36 |
| 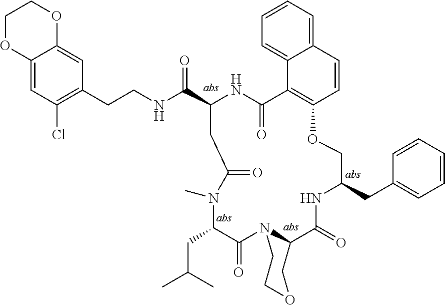 | 37 |
| 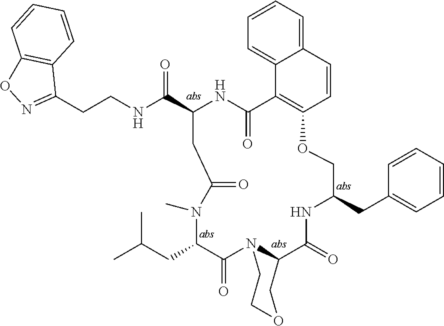 | 38 |
| 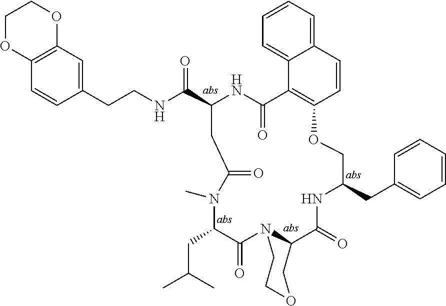 | 39 |
| 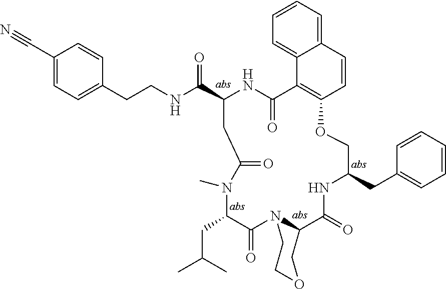 | 40 |
| 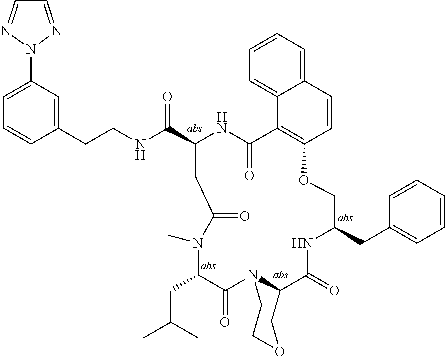 | 41 |
| 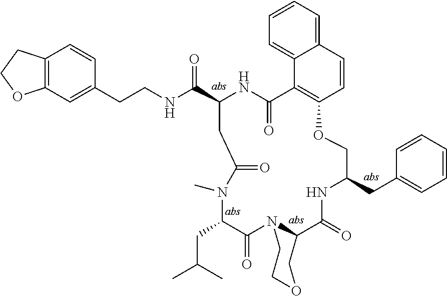 | 42 |
| 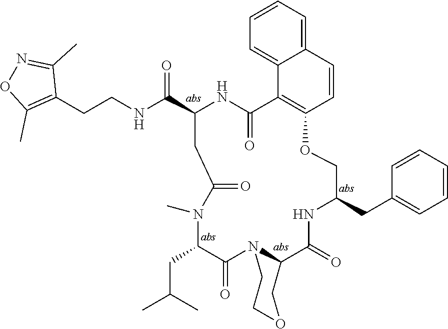 | 43 |
| 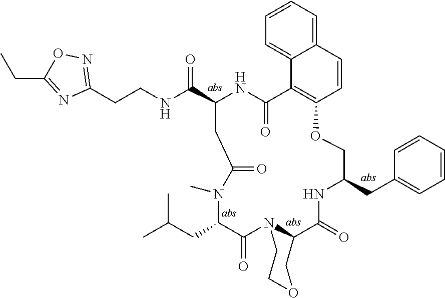 | 44 |
| 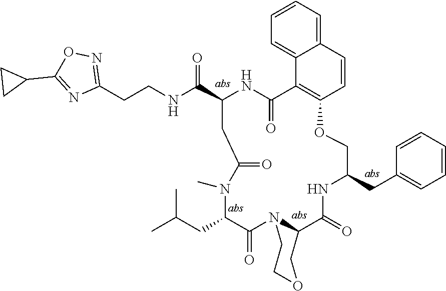 | 45 |
| 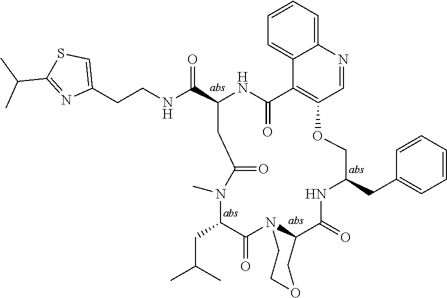 | 46 |
| 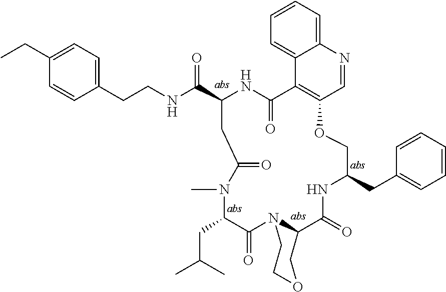 | 47 |
| 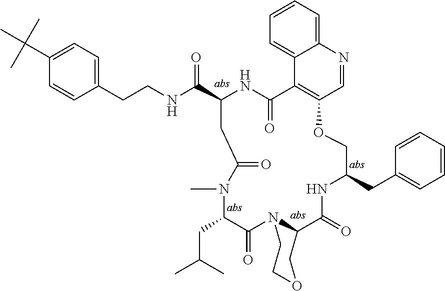 | 48 |
| 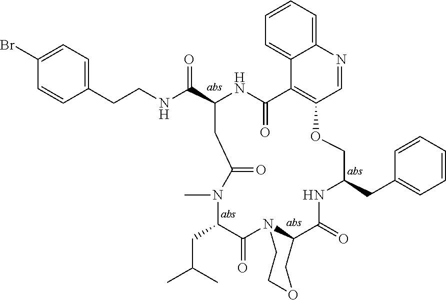 | 49 |
| 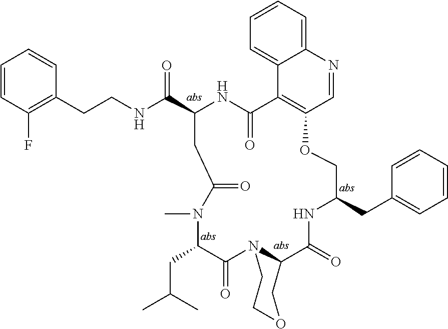 | 50 |
| 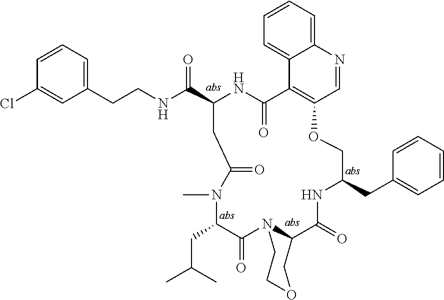 | 51 |
| 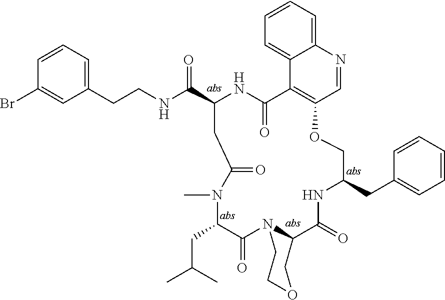 | 52 |
| 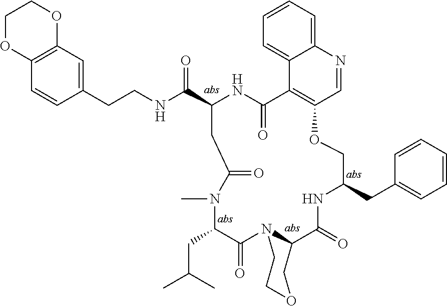 | 53 |
| 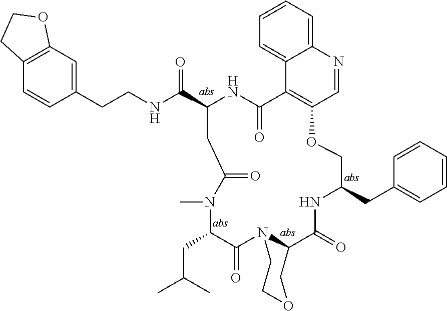 | 54 |
| 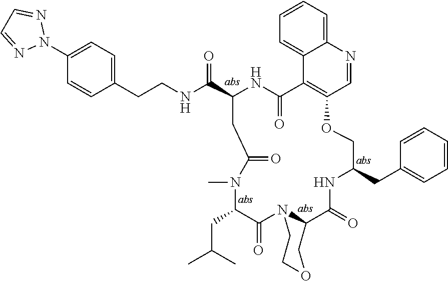 | 55 |
| 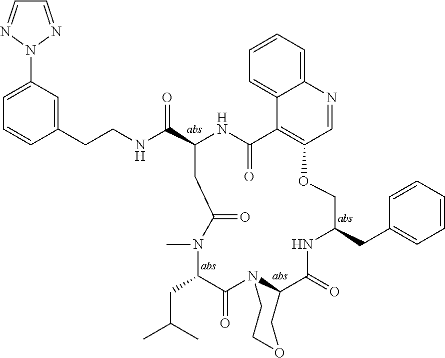 | 56 |
| 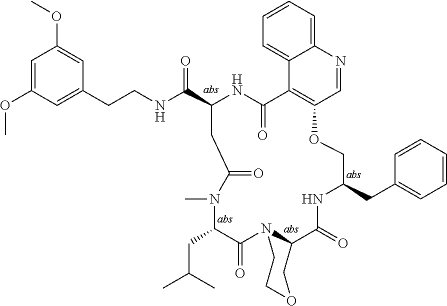 | 57 |
| 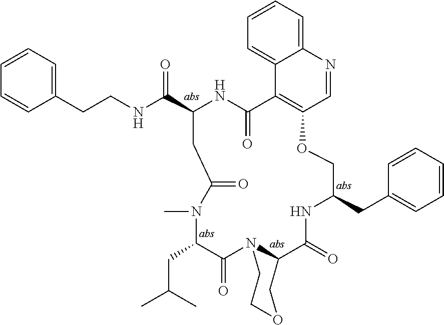 | 58 |
| 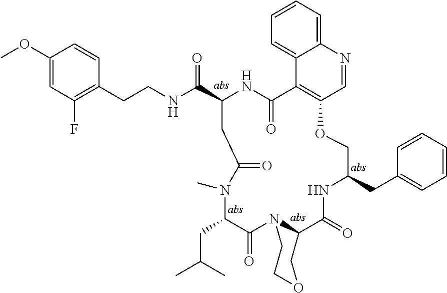 | 59 |
| 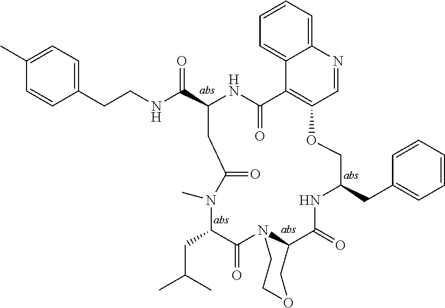 | 60 |
| 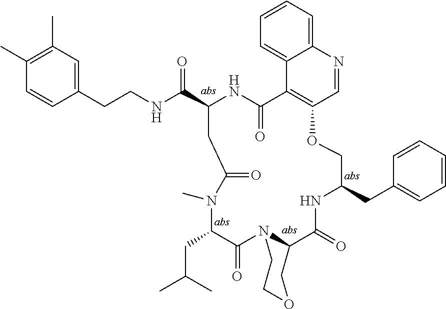 | 61 |
| 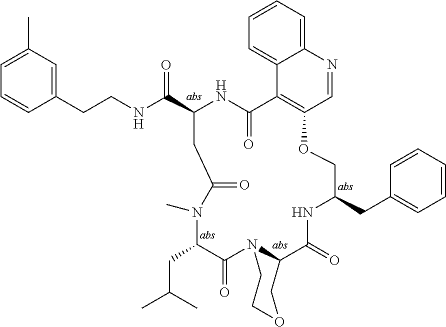 | 62 |
| 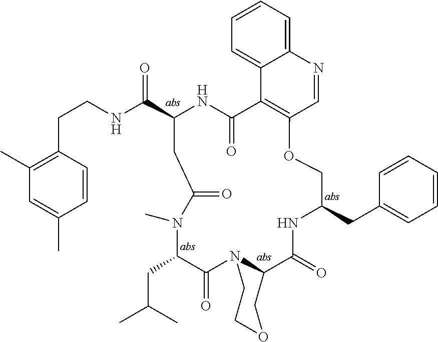 | 63 |
| 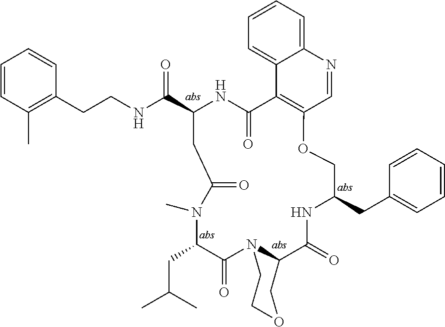 | 64 |
| 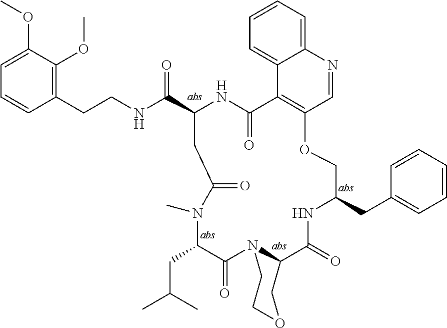 | 65 |
| 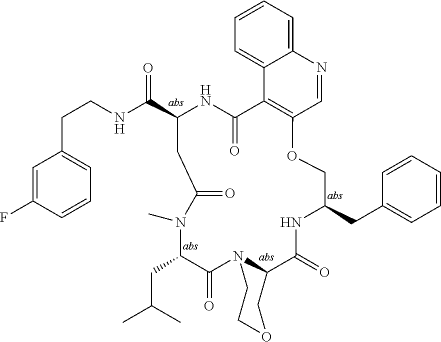 | 66 |
| 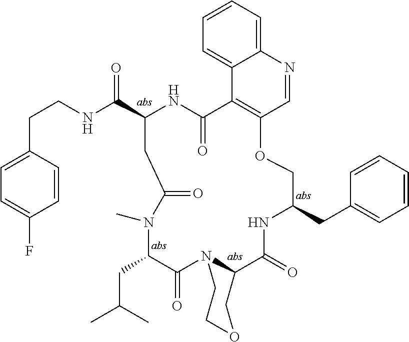 | 67 |
| 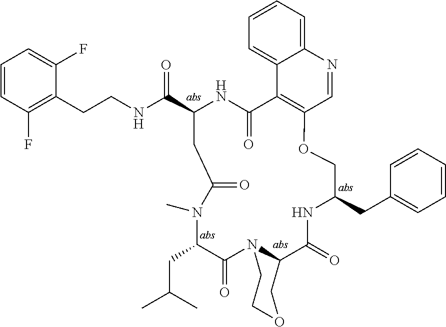 | 68 |
| 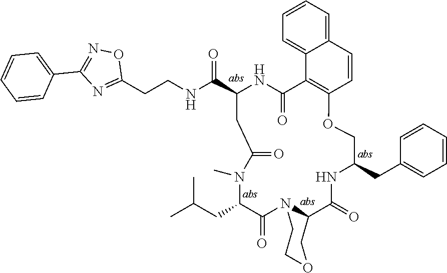 | 69 |
| 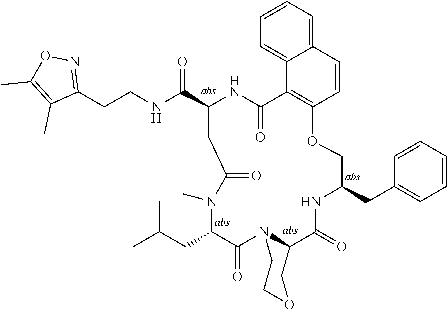 | 70 |
| 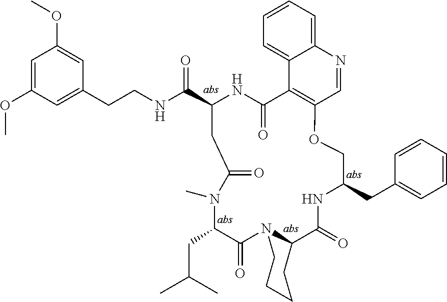 | 71 |
| 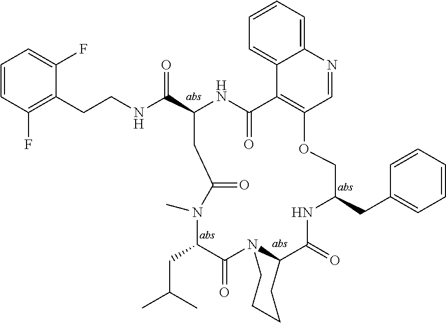 | 72 |
| 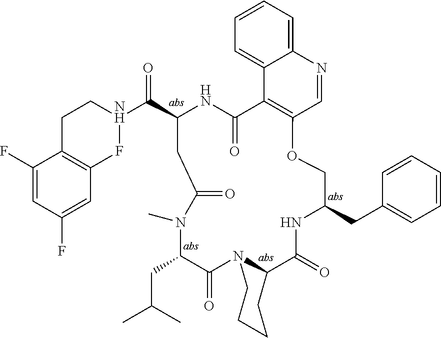 | 73 |
| 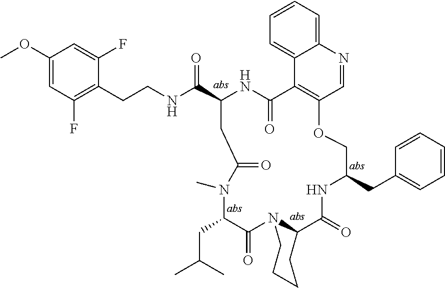 | 74 |
| 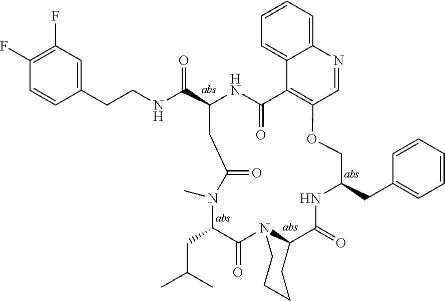 | 75 |
| 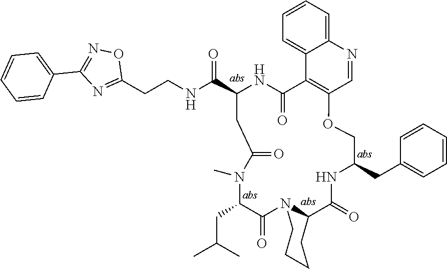 | 76 |
| 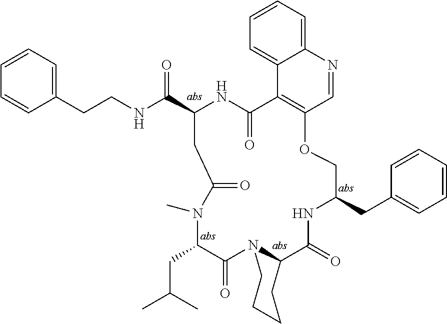 | 77 |
| 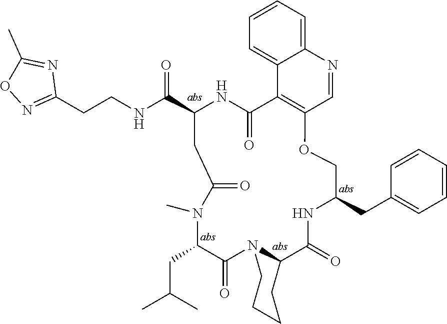 | 78 |
| 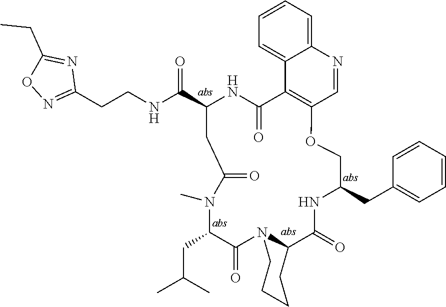 | 79 |
| 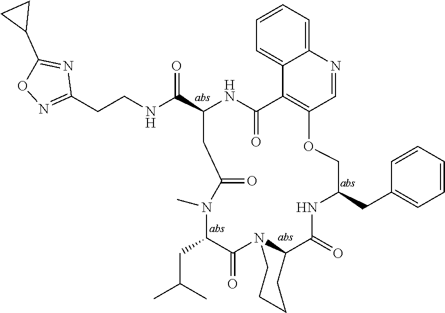 | 80 |
| 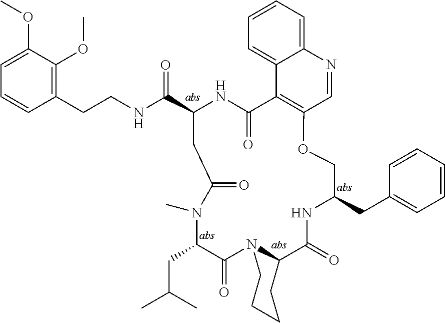 | 81 |
| 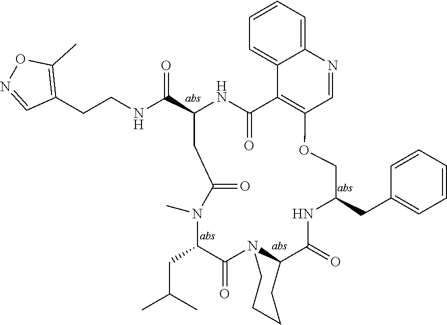 | 82 |
| 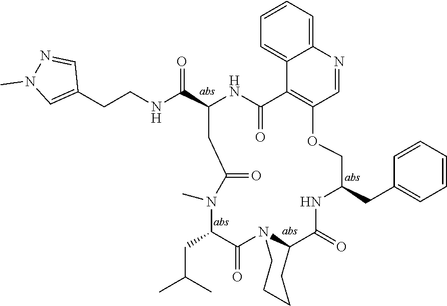 | 83 |
| 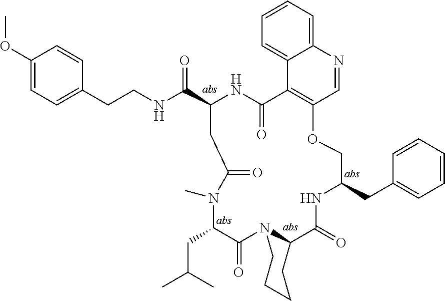 | 84 |
| 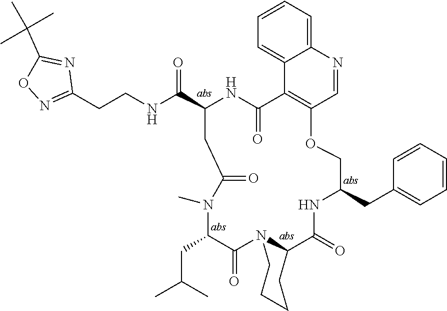 | 85 |
| 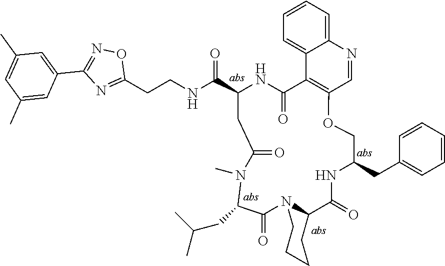 | 86 |
| 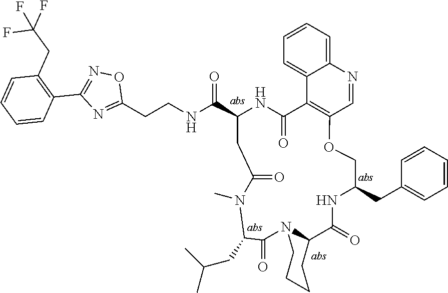 | 87 |
| 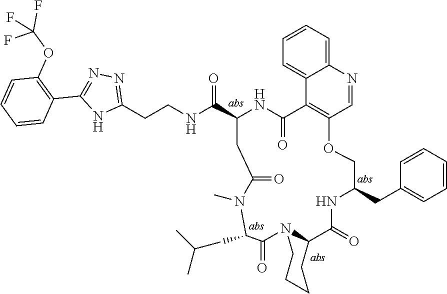 | 88 |
| 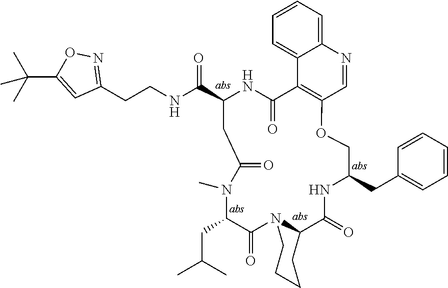 | 89 |
| 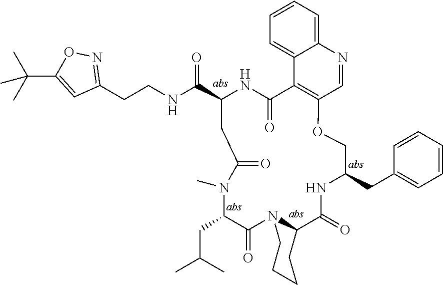 | 90 |
| 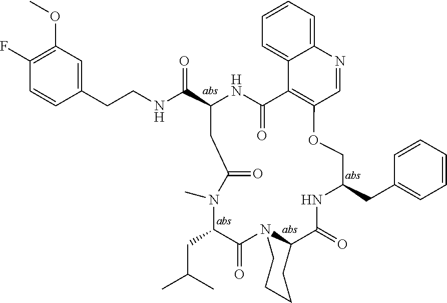 | 91 |
| 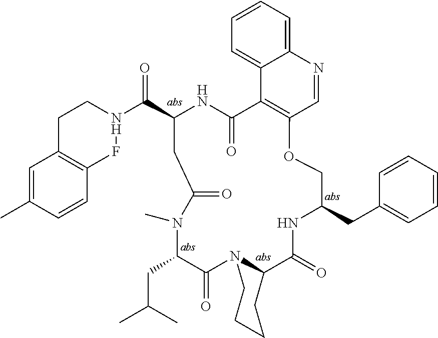 | 92 |
| 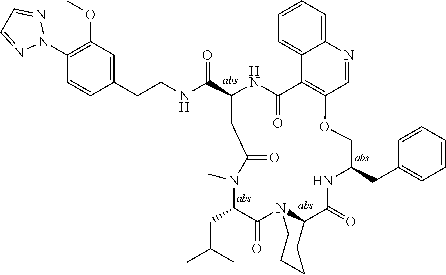 | 93 |
| 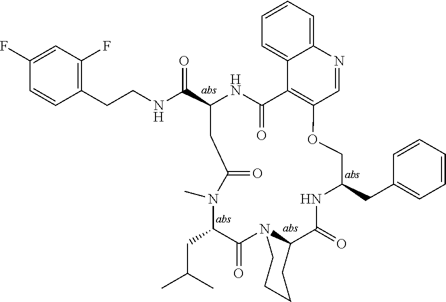 | 94 |
| 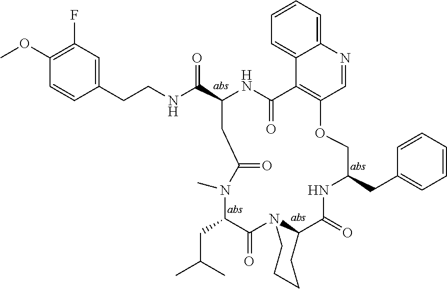 | 95 |
| 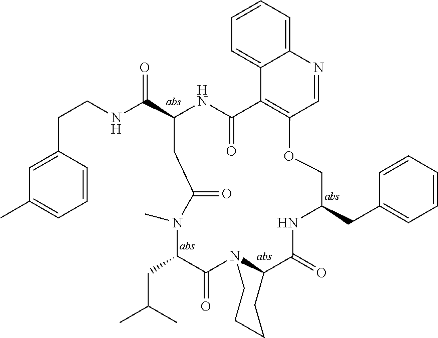 | 96 |
| 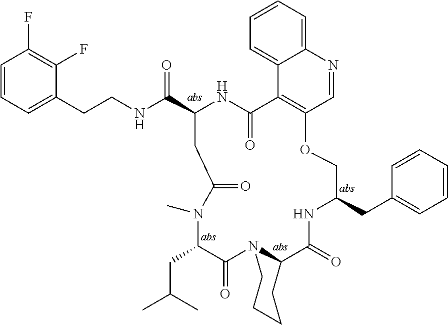 | 97 |
| 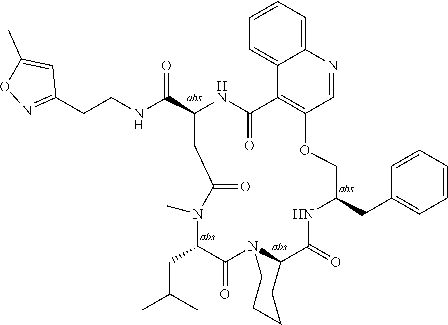 | 98 |
| 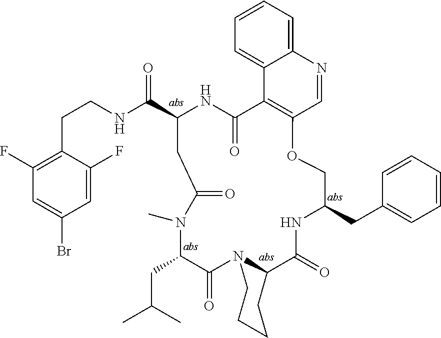 | 99 |
| 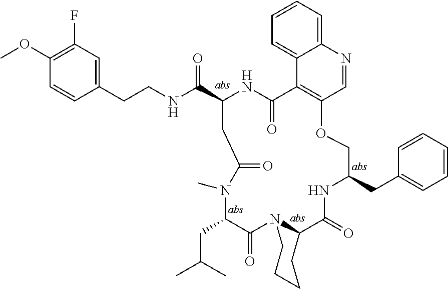 | 100 |
| 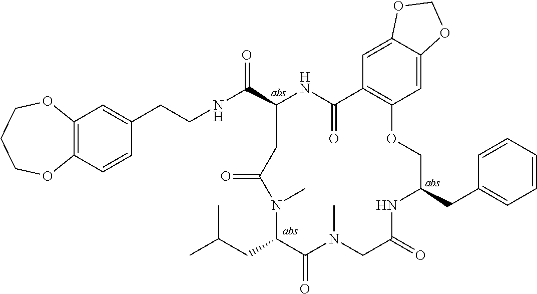 [SMILES: CC(C)C[C@H]1C(=O)N(C)CC(=O)N[C@H](Cc2ccccc2)COc2cc3c(cc2C(=O)N[C@H](C(=O)NCCc2ccc4c(c2)OCCCO4)CC(=O)N1C)OCO3] | 101 |
| 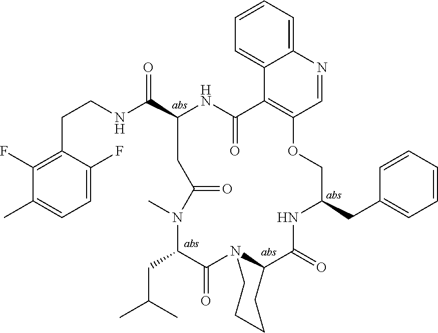 | 102 |
| 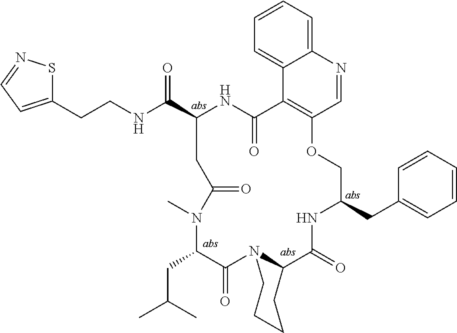 | 103 |
| 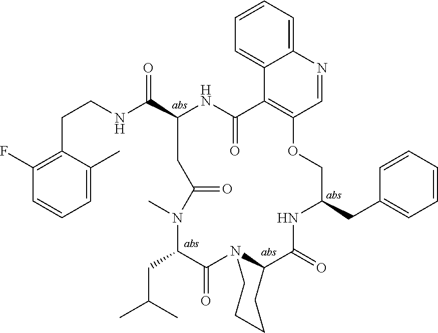 | 104 |
| 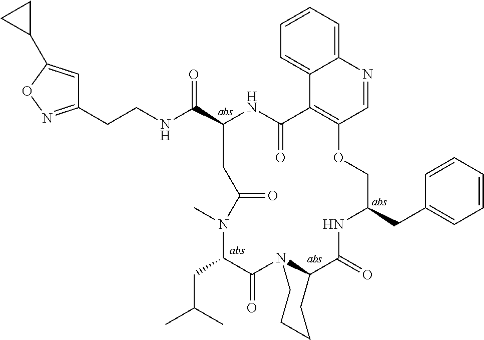 | 105 |
| 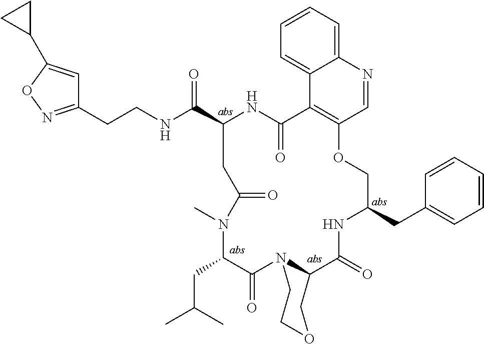 | 106 |
| 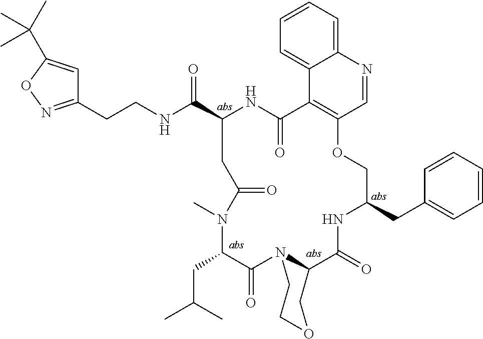 | 107 |
| 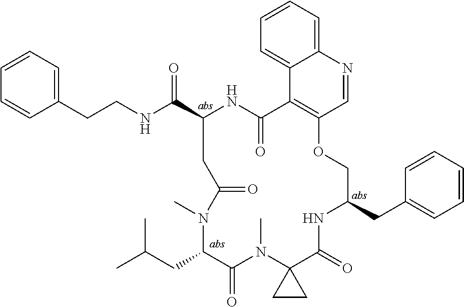 [SMILES: CC(C)C[C@H]1C(=O)N(C)C2(CC2)C(=O)N[C@H](Cc2ccccc2)COc2cnc3ccccc3c2C(=O)N[C@H](C(=O)NCCc2ccccc2)CC(=O)N1C] | 108 |
| 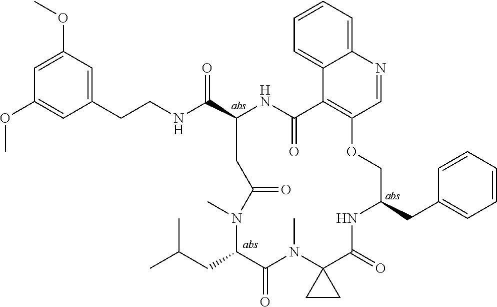 [SMILES: COc1cc(CCNC(=O)[C@@H]2CC(=O)N(C)[C@@H](CC(C)C)C(=O)N(C)C3(CC3)C(=O)N[C@H](Cc3ccccc3)COc3cnc4ccccc4c3C(=O)N2)cc(OC)c1] | 109 |
| 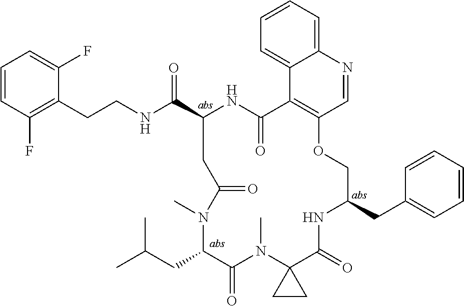 [SMILES: CC(C)C[C@H]1C(=O)N(C)C2(CC2)C(=O)N[C@H](Cc2ccccc2)COc2cnc3ccccc3c2C(=O)N[C@H](C(=O)NCCc2c(F)cccc2F)CC(=O)N1C] | 110 |
| 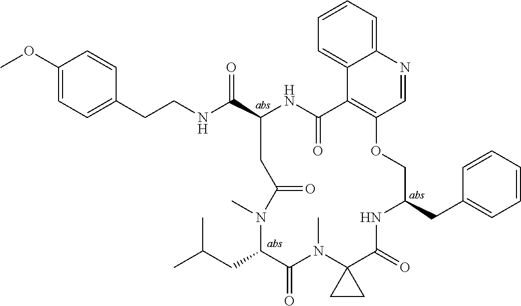 [SMILES: COc1ccc(CCNC(=O)[C@@H]2CC(=O)N(C)[C@@H](CC(C)C)C(=O)N(C)C3(CC3)C(=O)N[C@H](Cc3ccccc3)COc3cnc4ccccc4c3C(=O)N2)cc1] | 111 |
| 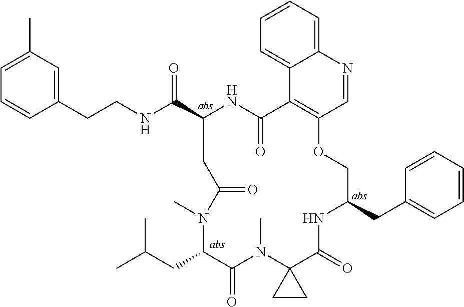 [SMILES: Cc1cccc(CCNC(=O)[C@@H]2CC(=O)N(C)[C@@H](CC(C)C)C(=O)N(C)C3(CC3)C(=O)N[C@H](Cc3ccccc3)COc3cnc4ccccc4c3C(=O)N2)c1] | 112 |
| 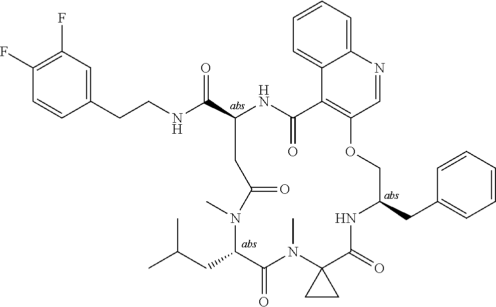 [SMILES: CC(C)C[C@H]1C(=O)N(C)C2(CC2)C(=O)N[C@H](Cc2ccccc2)COc2cnc3ccccc3c2C(=O)N[C@H](C(=O)NCCc2ccc(F)c(F)c2)CC(=O)N1C] | 113 |
| 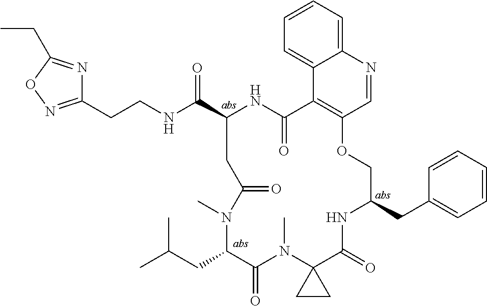 [SMILES: CCc1nc(CCNC(=O)[C@@H]2CC(=O)N(C)[C@@H](CC(C)C)C(=O)N(C)C3(CC3)C(=O)N[C@H](Cc3ccccc3)COc3cnc4ccccc4c3C(=O)N2)no1] | 114 |
| 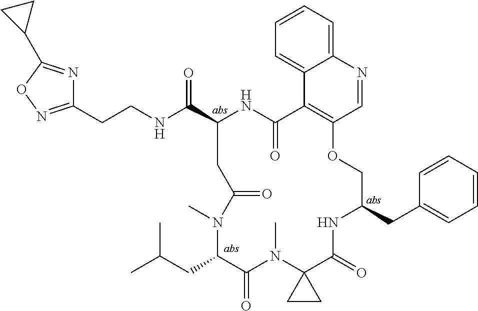 [SMILES: CC(C)C[C@H]1C(=O)N(C)C2(CC2)C(=O)N[C@H](Cc2ccccc2)COc2cnc3ccccc3c2C(=O)N[C@H](C(=O)NCCc2noc(C3CC3)n2)CC(=O)N1C] | 115 |
| 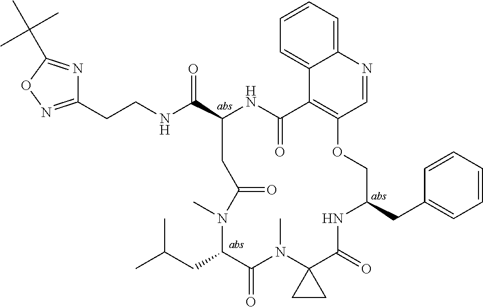 [SMILES: CC(C)C[C@H]1C(=O)N(C)C2(CC2)C(=O)N[C@H](Cc2ccccc2)COc2cnc3ccccc3c2C(=O)N[C@H](C(=O)NCCc2noc(C(C)(C)C)n2)CC(=O)N1C] | 116 |
| 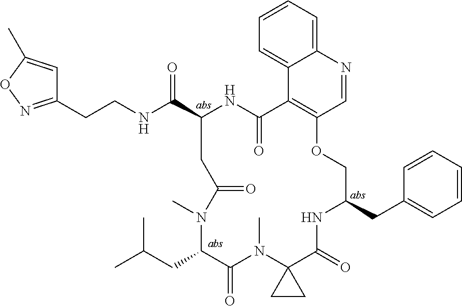 [SMILES: Cc1cc(CCNC(=O)[C@@H]2CC(=O)N(C)[C@@H](CC(C)C)C(=O)N(C)C3(CC3)C(=O)N[C@H](Cc3ccccc3)COc3cnc4ccccc4c3C(=O)N2)no1] | 117 |
| 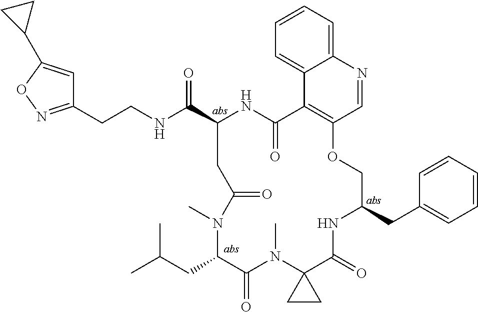 [SMILES: CC(C)C[C@H]1C(=O)N(C)C2(CC2)C(=O)N[C@H](Cc2ccccc2)COc2cnc3ccccc3c2C(=O)N[C@H](C(=O)NCCc2cc(C3CC3)on2)CC(=O)N1C] | 118 |
| 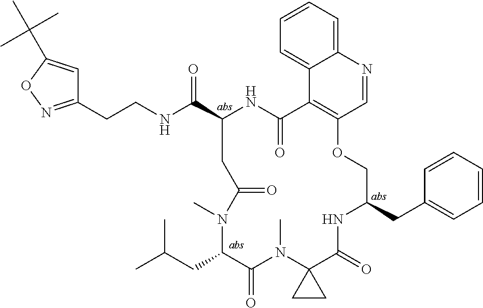 [SMILES: CC(C)C[C@H]1C(=O)N(C)C2(CC2)C(=O)N[C@H](Cc2ccccc2)COc2cnc3ccccc3c2C(=O)N[C@H](C(=O)NCCc2cc(C(C)(C)C)on2)CC(=O)N1C] | 119 |
| 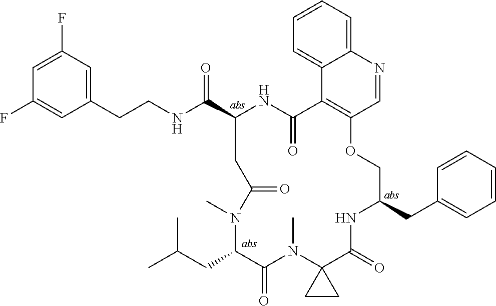 [SMILES: CC(C)C[C@H]1C(=O)N(C)C2(CC2)C(=O)N[C@H](Cc2ccccc2)COc2cnc3ccccc3c2C(=O)N[C@H](C(=O)NCCc2cc(F)cc(F)c2)CC(=O)N1C] | 120 |
| 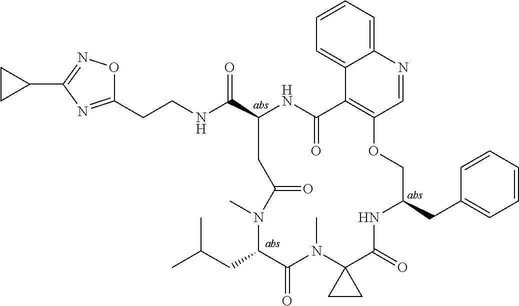 [SMILES: CC(C)C[C@H]1C(=O)N(C)C2(CC2)C(=O)N[C@H](Cc2ccccc2)COc2cnc3ccccc3c2C(=O)N[C@H](C(=O)NCCc2nc(C3CC3)no2)CC(=O)N1C] | 121 |
| 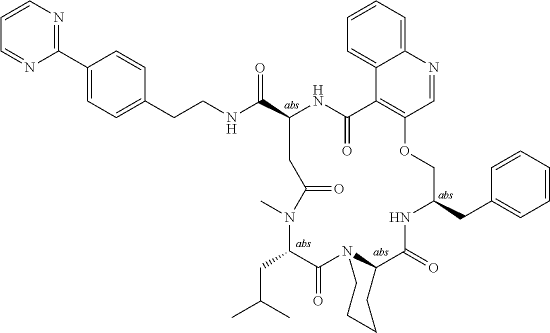 | 122 |
| 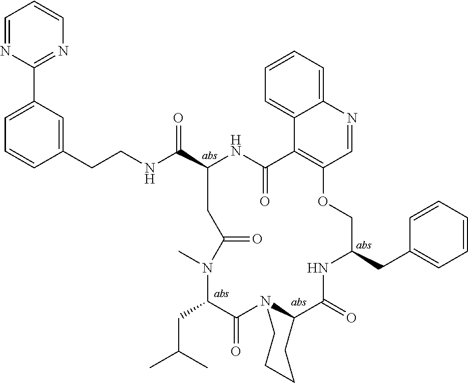 | 123 |
| 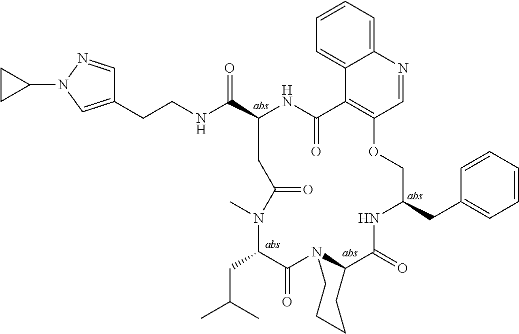 | 124 |
| 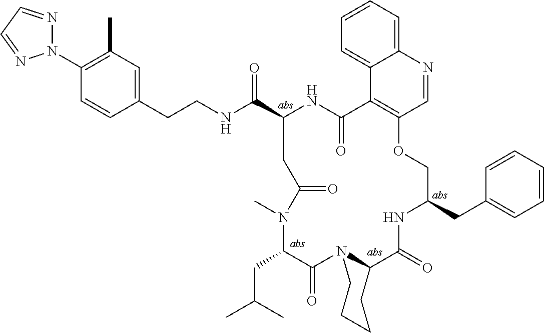 | 125 |
| 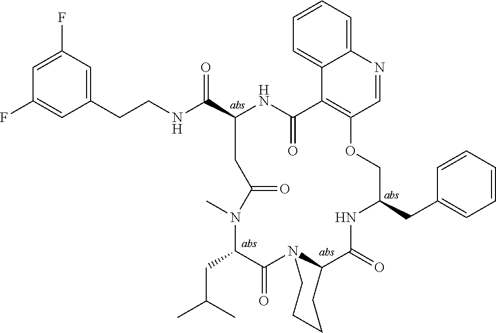 | 126 |
| 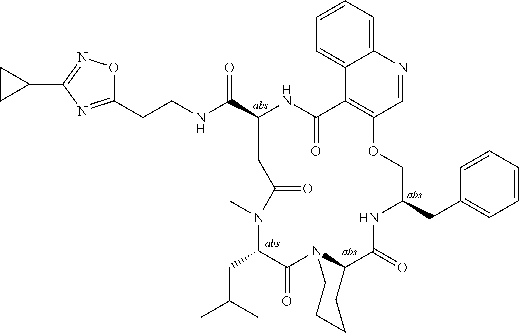 | 127 |
| 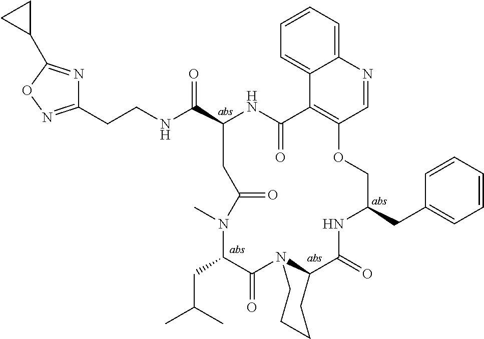 | 128 |
| 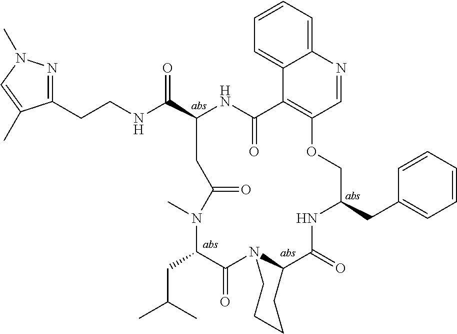 | 129 |
| 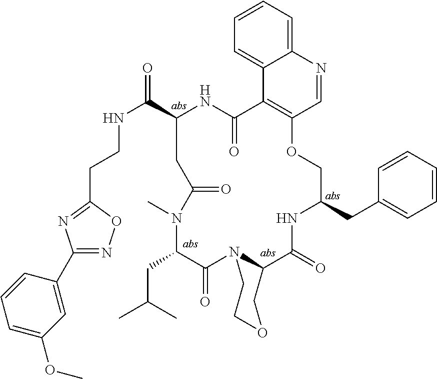 | 130 |
| 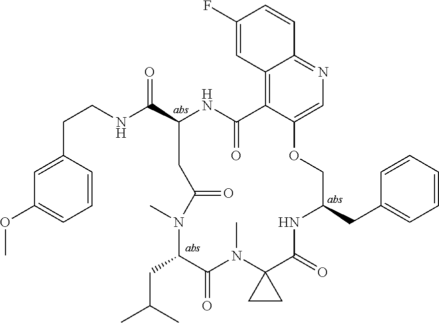 [SMILES: COc1cccc(CCNC(=O)[C@@H]2CC(=O)N(C)[C@@H](CC(C)C)C(=O)N(C)C3(CC3)C(=O)N[C@H](Cc3ccccc3)COc3cnc4ccc(F)cc4c3C(=O)N2)c1] | 131 |
| 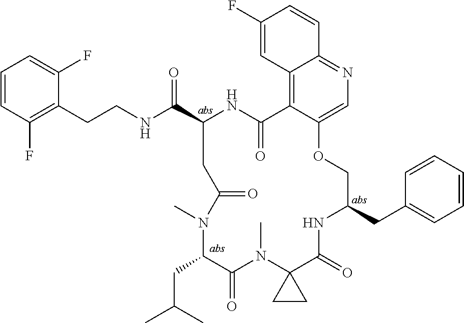 [SMILES: CC(C)C[C@H]1C(=O)N(C)C2(CC2)C(=O)N[C@H](Cc2ccccc2)COc2cnc3ccc(F)cc3c2C(=O)N[C@H](C(=O)NCCc2c(F)cccc2F)CC(=O)N1C] | 132 |
| 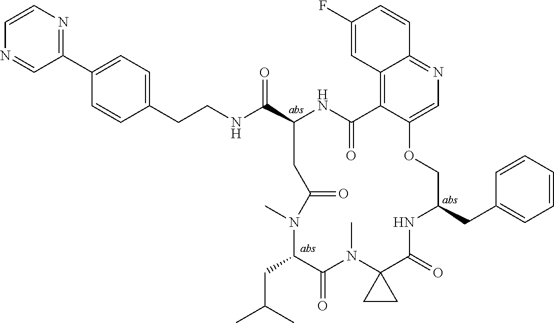 [SMILES: CC(C)C[C@H]1C(=O)N(C)C2(CC2)C(=O)N[C@H](Cc2ccccc2)COc2cnc3ccc(F)cc3c2C(=O)N[C@H](C(=O)NCCc2ccc(-c3cnccn3)cc2)CC(=O)N1C] | 133 |
| 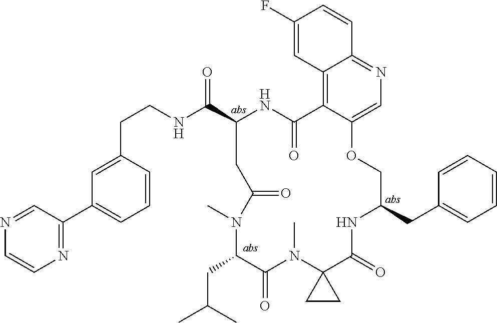 [SMILES: CC(C)C[C@H]1C(=O)N(C)C2(CC2)C(=O)N[C@H](Cc2ccccc2)COc2cnc3ccc(F)cc3c2C(=O)N[C@H](C(=O)NCCc2cccc(-c3cnccn3)c2)CC(=O)N1C] | 134 |
| 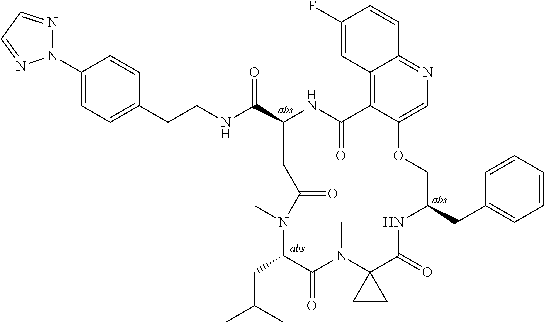 [SMILES: CC(C)C[C@H]1C(=O)N(C)C2(CC2)C(=O)N[C@H](Cc2ccccc2)COc2cnc3ccc(F)cc3c2C(=O)N[C@H](C(=O)NCCc2ccc(-n3nccn3)cc2)CC(=O)N1C] | 135 |
| 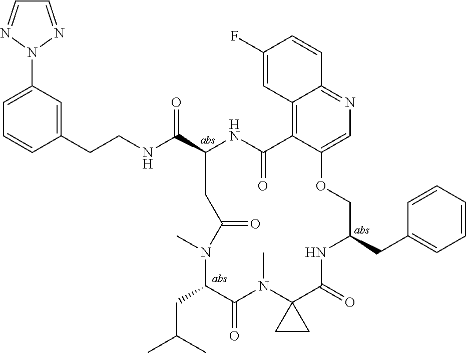 [SMILES: CC(C)C[C@H]1C(=O)N(C)C2(CC2)C(=O)N[C@H](Cc2ccccc2)COc2cnc3ccc(F)cc3c2C(=O)N[C@H](C(=O)NCCc2cccc(-n3nccn3)c2)CC(=O)N1C] | 136 |
| 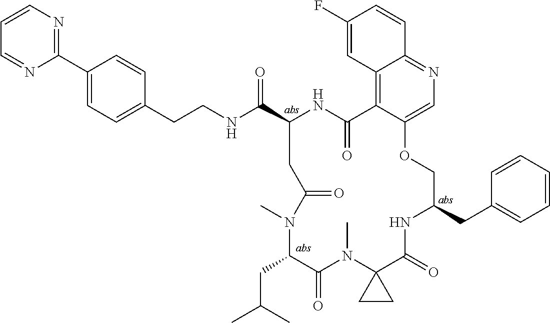 [SMILES: CC(C)C[C@H]1C(=O)N(C)C2(CC2)C(=O)N[C@H](Cc2ccccc2)COc2cnc3ccc(F)cc3c2C(=O)N[C@H](C(=O)NCCc2ccc(-c3ncccn3)cc2)CC(=O)N1C] | 137 |
| 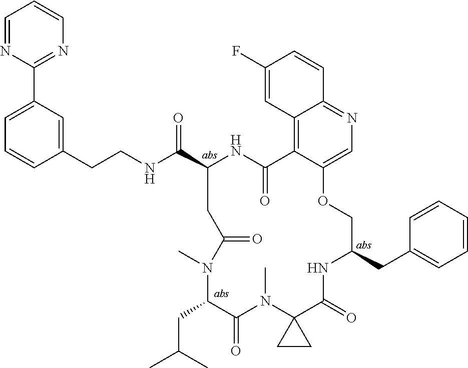 [SMILES: CC(C)C[C@H]1C(=O)N(C)C2(CC2)C(=O)N[C@H](Cc2ccccc2)COc2cnc3ccc(F)cc3c2C(=O)N[C@H](C(=O)NCCc2cccc(-c3ncccn3)c2)CC(=O)N1C] | 138 |
| 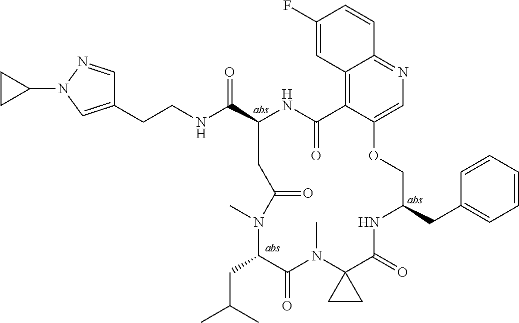 [SMILES: CC(C)C[C@H]1C(=O)N(C)C2(CC2)C(=O)N[C@H](Cc2ccccc2)COc2cnc3ccc(F)cc3c2C(=O)N[C@H](C(=O)NCCc2cnn(C3CC3)c2)CC(=O)N1C] | 139 |
| 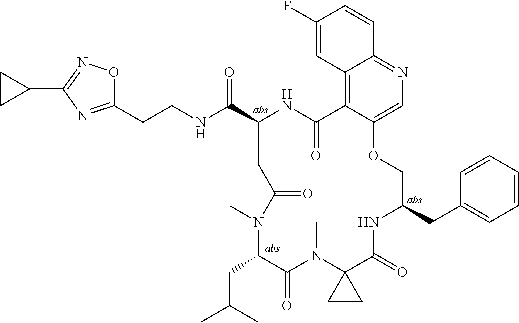 [SMILES: CC(C)C[C@H]1C(=O)N(C)C2(CC2)C(=O)N[C@H](Cc2ccccc2)COc2cnc3ccc(F)cc3c2C(=O)N[C@H](C(=O)NCCc2nc(C3CC3)no2)CC(=O)N1C] | 140 |
| 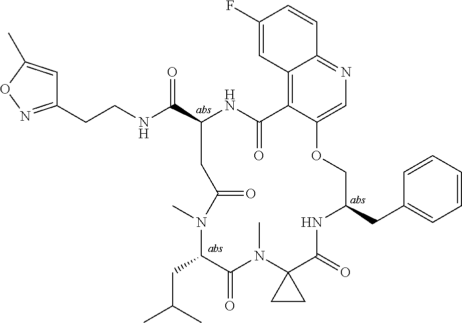 [SMILES: Cc1cc(CCNC(=O)[C@@H]2CC(=O)N(C)[C@@H](CC(C)C)C(=O)N(C)C3(CC3)C(=O)N[C@H](Cc3ccccc3)COc3cnc4ccc(F)cc4c3C(=O)N2)no1] | 141 |
| 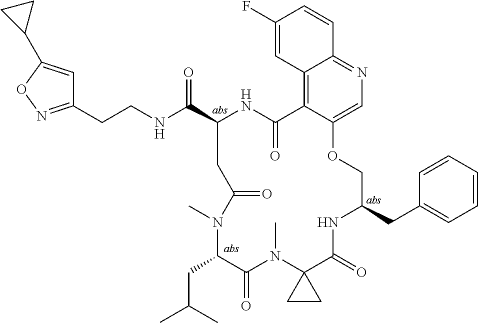 [SMILES: CC(C)C[C@H]1C(=O)N(C)C2(CC2)C(=O)N[C@H](Cc2ccccc2)COc2cnc3ccc(F)cc3c2C(=O)N[C@H](C(=O)NCCc2cc(C3CC3)on2)CC(=O)N1C] | 142 |
| 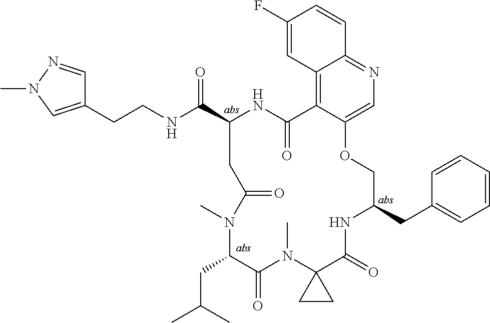 [SMILES: CC(C)C[C@H]1C(=O)N(C)C2(CC2)C(=O)N[C@H](Cc2ccccc2)COc2cnc3ccc(F)cc3c2C(=O)N[C@H](C(=O)NCCc2cnn(C)c2)CC(=O)N1C] | 143 |
| 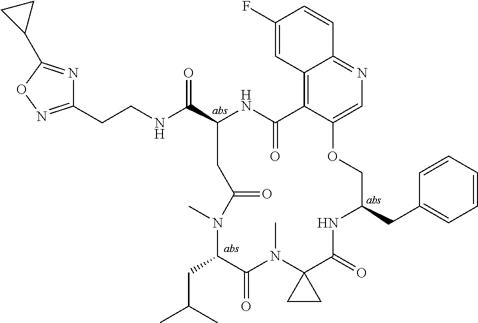 [SMILES: CC(C)C[C@H]1C(=O)N(C)C2(CC2)C(=O)N[C@H](Cc2ccccc2)COc2cnc3ccc(F)cc3c2C(=O)N[C@H](C(=O)NCCc2noc(C3CC3)n2)CC(=O)N1C] | 144 |
| 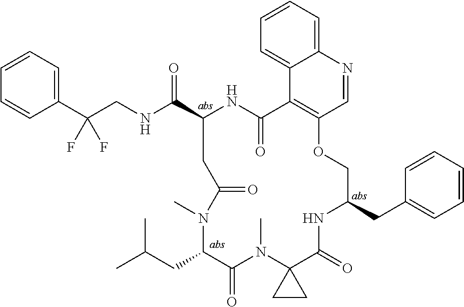 [SMILES: CC(C)C[C@H]1C(=O)N(C)C2(CC2)C(=O)N[C@H](Cc2ccccc2)COc2cnc3ccccc3c2C(=O)N[C@H](C(=O)NCC(F)(F)c2ccccc2)CC(=O)N1C] | 145 |
| 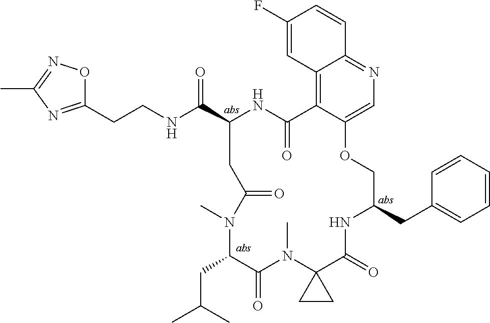 [SMILES: Cc1noc(CCNC(=O)[C@@H]2CC(=O)N(C)[C@@H](CC(C)C)C(=O)N(C)C3(CC3)C(=O)N[C@H](Cc3ccccc3)COc3cnc4ccc(F)cc4c3C(=O)N2)n1] | 146 |
| 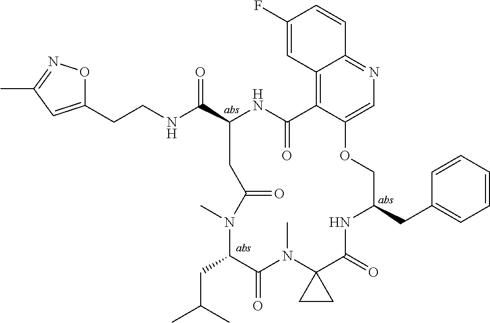 [SMILES: Cc1cc(CCNC(=O)[C@@H]2CC(=O)N(C)[C@@H](CC(C)C)C(=O)N(C)C3(CC3)C(=O)N[C@H](Cc3ccccc3)COc3cnc4ccc(F)cc4c3C(=O)N2)on1] | 147 |
| 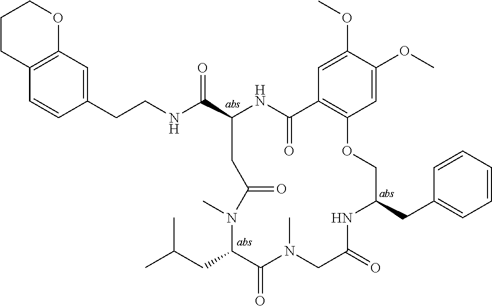 [SMILES: COc1cc2c(cc1OC)C(=O)N[C@H](C(=O)NCCc1ccc3c(c1)OCCC3)CC(=O)N(C)[C@@H](CC(C)C)C(=O)N(C)CC(=O)N[C@H](Cc1ccccc1)CO2] | 148 |
| 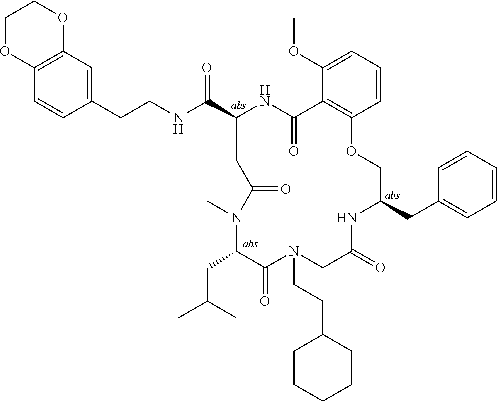 [SMILES: COc1cccc2c1C(=O)N[C@H](C(=O)NCCc1ccc3c(c1)OCCO3)CC(=O)N(C)[C@@H](CC(C)C)C(=O)N(CCC1CCCCC1)CC(=O)N[C@H](Cc1ccccc1)CO2] | 149 |
| 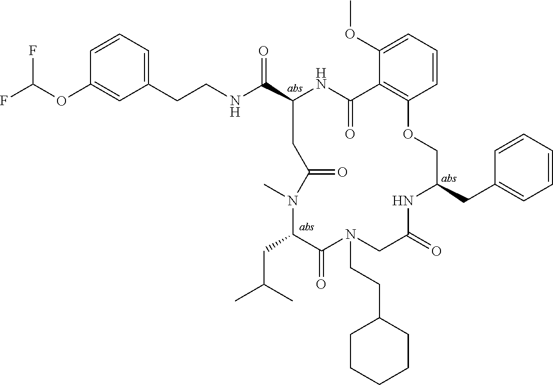 [SMILES: COc1cccc2c1C(=O)N[C@H](C(=O)NCCc1cccc(OC(F)F)c1)CC(=O)N(C)[C@@H](CC(C)C)C(=O)N(CCC1CCCCC1)CC(=O)N[C@H](Cc1ccccc1)CO2] | 150 |
| 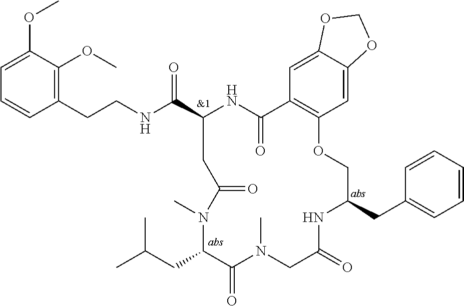 [SMILES: COc1cccc(CCNC(=O)[C@@H]2CC(=O)N(C)[C@@H](CC(C)C)C(=O)N(C)CC(=O)N[C@H](Cc3ccccc3)COc3cc4c(cc3C(=O)N2)OCO4)c1OC] | 151 |
| 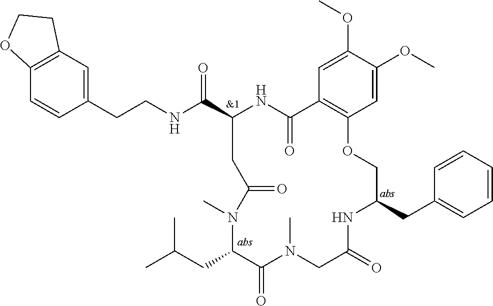 [SMILES: COc1cc2c(cc1OC)C(=O)N[C@H](C(=O)NCCc1ccc3c(c1)CCO3)CC(=O)N(C)[C@@H](CC(C)C)C(=O)N(C)CC(=O)N[C@H](Cc1ccccc1)CO2] | 152 |
| 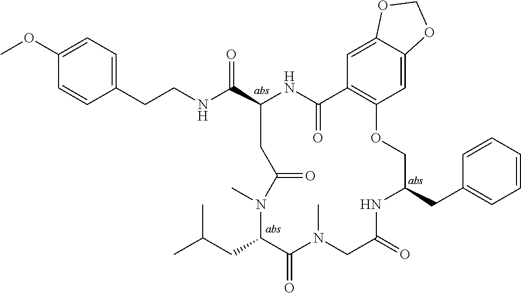 [SMILES: COc1ccc(CCNC(=O)[C@@H]2CC(=O)N(C)[C@@H](CC(C)C)C(=O)N(C)CC(=O)N[C@H](Cc3ccccc3)COc3cc4c(cc3C(=O)N2)OCO4)cc1] | 153 |
| 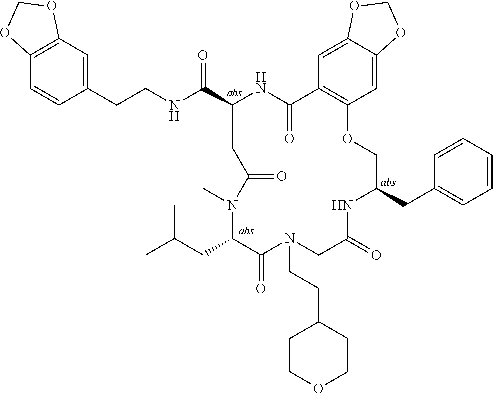 [SMILES: CC(C)C[C@H]1C(=O)N(CCC2CCOCC2)CC(=O)N[C@H](Cc2ccccc2)COc2cc3c(cc2C(=O)N[C@H](C(=O)NCCc2ccc4c(c2)OCO4)CC(=O)N1C)OCO3] | 154 |
| 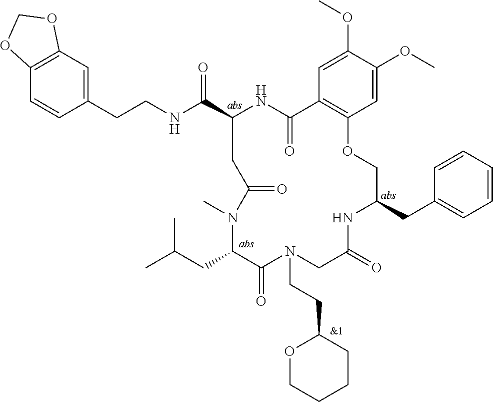 [SMILES: COc1cc2c(cc1OC)C(=O)N[C@H](C(=O)NCCc1ccc3c(c1)OCO3)CC(=O)N(C)[C@@H](CC(C)C)C(=O)N(CC[C@H]1CCCCO1)CC(=O)N[C@H](Cc1ccccc1)CO2] | 155 |
| 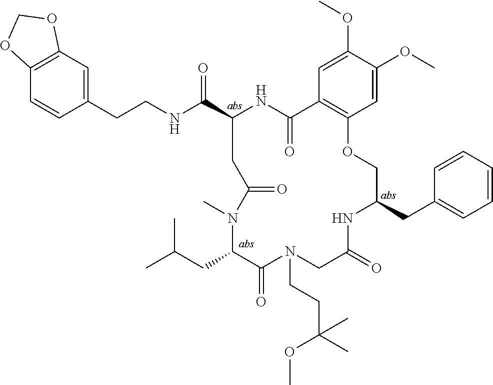 [SMILES: COc1cc2c(cc1OC)C(=O)N[C@H](C(=O)NCCc1ccc3c(c1)OCO3)CC(=O)N(C)[C@@H](CC(C)C)C(=O)N(CCC(C)(C)OC)CC(=O)N[C@H](Cc1ccccc1)CO2] | 156 |
| 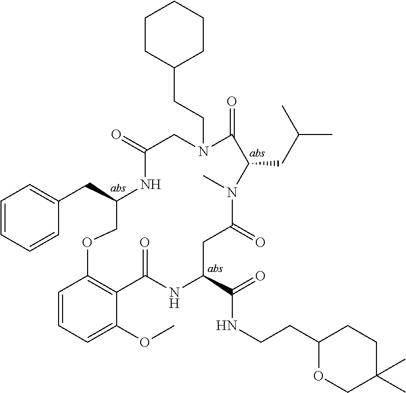 [SMILES: COc1cccc2c1C(=O)N[C@H](C(=O)NCCC1CCC(C)(C)CO1)CC(=O)N(C)[C@@H](CC(C)C)C(=O)N(CCC1CCCCC1)CC(=O)N[C@H](Cc1ccccc1)CO2] | 157 |
| 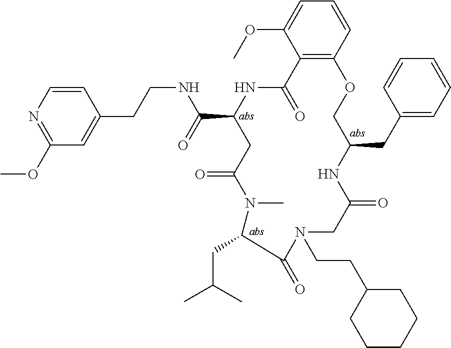 [SMILES: COc1cc(CCNC(=O)[C@@H]2CC(=O)N(C)[C@@H](CC(C)C)C(=O)N(CCC3CCCCC3)CC(=O)N[C@H](Cc3ccccc3)COc3cccc(OC)c3C(=O)N2)ccn1] | 158 |
| 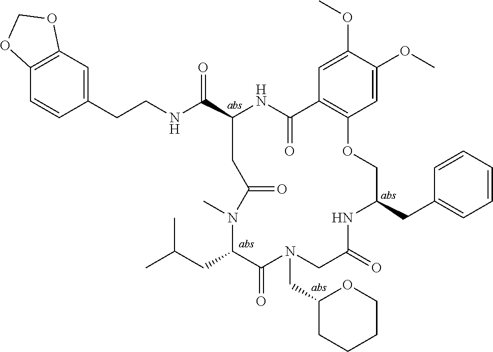 [SMILES: COc1cc2c(cc1OC)C(=O)N[C@H](C(=O)NCCc1ccc3c(c1)OCO3)CC(=O)N(C)[C@@H](CC(C)C)C(=O)N(C[C@H]1CCCCO1)CC(=O)N[C@H](Cc1ccccc1)CO2] | 159 |
| 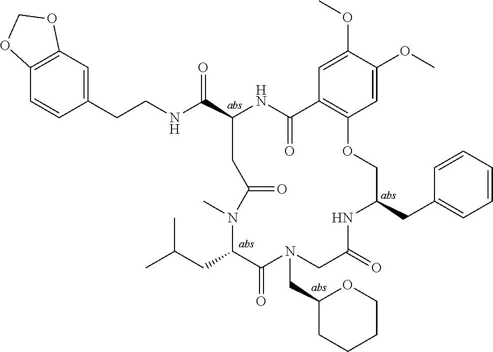 [SMILES: COc1cc2c(cc1OC)C(=O)N[C@H](C(=O)NCCc1ccc3c(c1)OCO3)CC(=O)N(C)[C@@H](CC(C)C)C(=O)N(C[C@@H]1CCCCO1)CC(=O)N[C@H](Cc1ccccc1)CO2] | 160 |
| 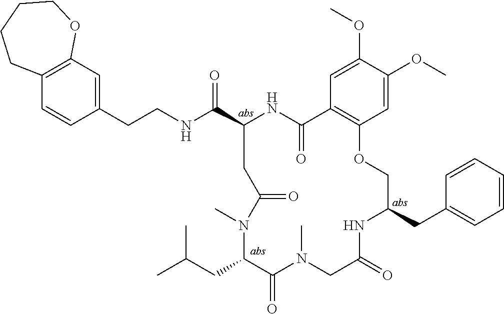 [SMILES: COc1cc2c(cc1OC)C(=O)N[C@H](C(=O)NCCc1ccc3c(c1)OCCCC3)CC(=O)N(C)[C@@H](CC(C)C)C(=O)N(C)CC(=O)N[C@H](Cc1ccccc1)CO2] | 161 |
| 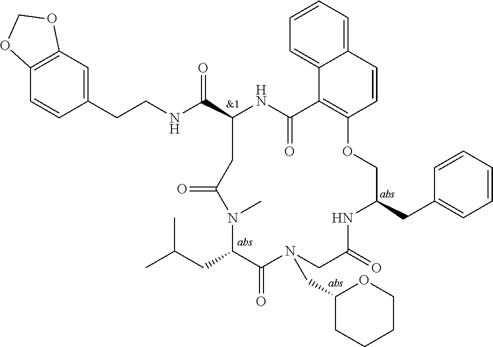 [SMILES: CC(C)C[C@H]1C(=O)N(C[C@H]2CCCCO2)CC(=O)N[C@H](Cc2ccccc2)COc2ccc3ccccc3c2C(=O)N[C@H](C(=O)NCCc2ccc3c(c2)OCO3)CC(=O)N1C] | 162 |
| 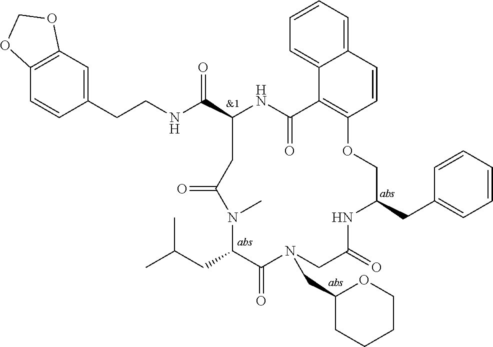 [SMILES: CC(C)C[C@H]1C(=O)N(C[C@@H]2CCCCO2)CC(=O)N[C@H](Cc2ccccc2)COc2ccc3ccccc3c2C(=O)N[C@H](C(=O)NCCc2ccc3c(c2)OCO3)CC(=O)N1C] | 163 |
| 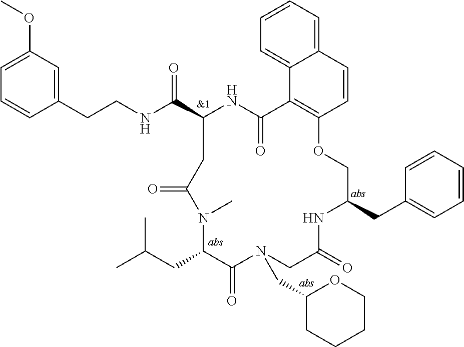 [SMILES: COc1cccc(CCNC(=O)[C@@H]2CC(=O)N(C)[C@@H](CC(C)C)C(=O)N(C[C@H]3CCCCO3)CC(=O)N[C@H](Cc3ccccc3)COc3ccc4ccccc4c3C(=O)N2)c1] | 164 |
| 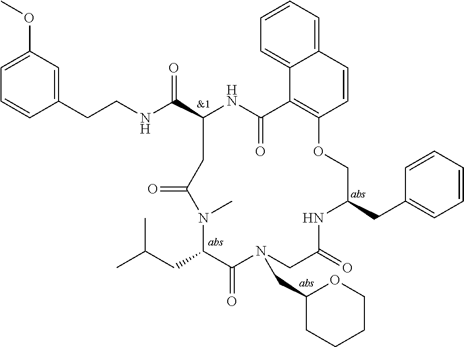 [SMILES: COc1cccc(CCNC(=O)[C@@H]2CC(=O)N(C)[C@@H](CC(C)C)C(=O)N(C[C@@H]3CCCCO3)CC(=O)N[C@H](Cc3ccccc3)COc3ccc4ccccc4c3C(=O)N2)c1] | 165 |
| 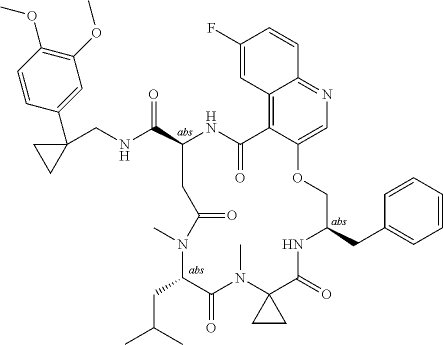 [SMILES: COc1ccc(C2(CNC(=O)[C@@H]3CC(=O)N(C)[C@@H](CC(C)C)C(=O)N(C)C4(CC4)C(=O)N[C@H](Cc4ccccc4)COc4cnc5ccc(F)cc5c4C(=O)N3)CC2)cc1OC] | 166 |
| 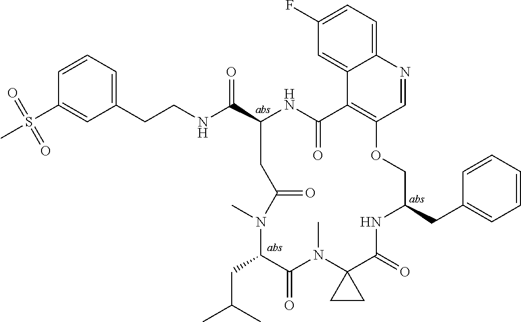 [SMILES: CC(C)C[C@H]1C(=O)N(C)C2(CC2)C(=O)N[C@H](Cc2ccccc2)COc2cnc3ccc(F)cc3c2C(=O)N[C@H](C(=O)NCCc2cccc(S(C)(=O)=O)c2)CC(=O)N1C] | 167 |
| 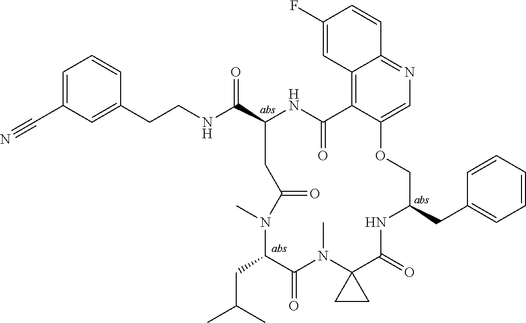 [SMILES: CC(C)C[C@H]1C(=O)N(C)C2(CC2)C(=O)N[C@H](Cc2ccccc2)COc2cnc3ccc(F)cc3c2C(=O)N[C@H](C(=O)NCCc2cccc(C#N)c2)CC(=O)N1C] | 168 |
| 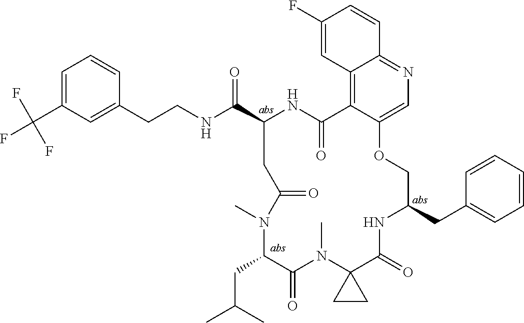 [SMILES: CC(C)C[C@H]1C(=O)N(C)C2(CC2)C(=O)N[C@H](Cc2ccccc2)COc2cnc3ccc(F)cc3c2C(=O)N[C@H](C(=O)NCCc2cccc(C(F)(F)F)c2)CC(=O)N1C] | 169 |
| 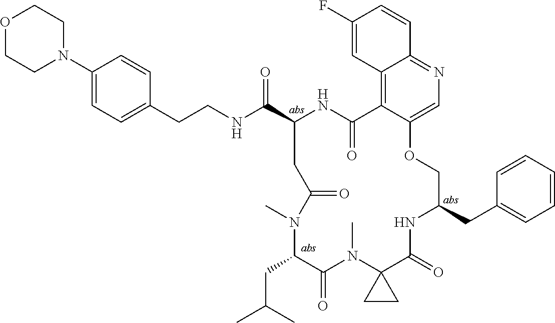 [SMILES: CC(C)C[C@H]1C(=O)N(C)C2(CC2)C(=O)N[C@H](Cc2ccccc2)COc2cnc3ccc(F)cc3c2C(=O)N[C@H](C(=O)NCCc2ccc(N3CCOCC3)cc2)CC(=O)N1C] | 170 |
| 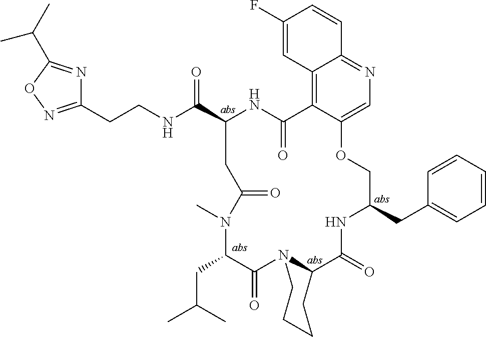 | 171 |
| 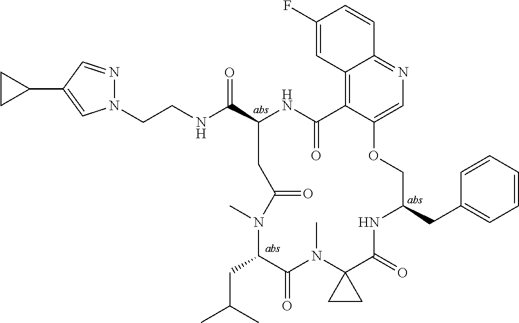 [SMILES: CC(C)C[C@H]1C(=O)N(C)C2(CC2)C(=O)N[C@H](Cc2ccccc2)COc2cnc3ccc(F)cc3c2C(=O)N[C@H](C(=O)NCCn2cc(C3CC3)cn2)CC(=O)N1C] | 172 |
| 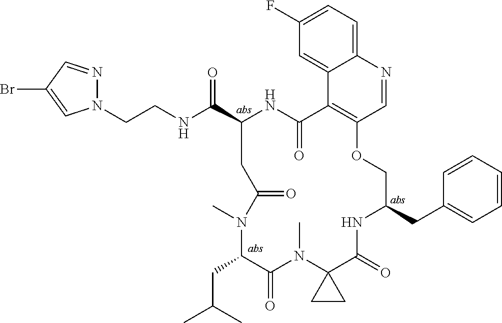 [SMILES: CC(C)C[C@H]1C(=O)N(C)C2(CC2)C(=O)N[C@H](Cc2ccccc2)COc2cnc3ccc(F)cc3c2C(=O)N[C@H](C(=O)NCCn2cc(Br)cn2)CC(=O)N1C] | 173 |
| 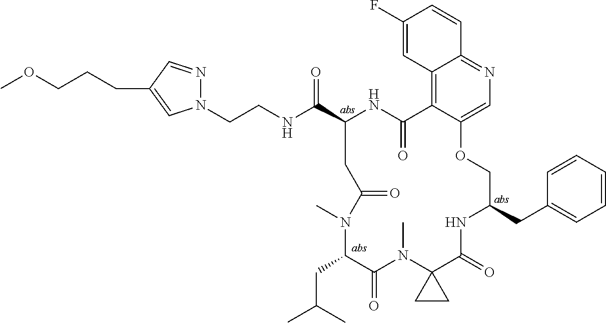 [SMILES: COCCCc1cnn(CCNC(=O)[C@@H]2CC(=O)N(C)[C@@H](CC(C)C)C(=O)N(C)C3(CC3)C(=O)N[C@H](Cc3ccccc3)COc3cnc4ccc(F)cc4c3C(=O)N2)c1] | 174 |
| 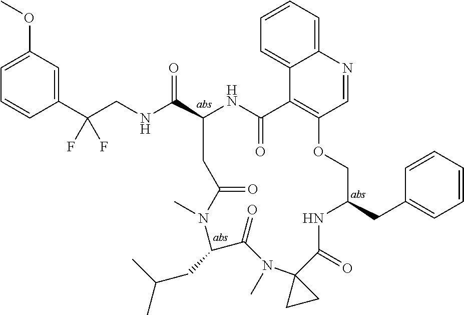 [SMILES: COc1cccc(C(F)(F)CNC(=O)[C@@H]2CC(=O)N(C)[C@@H](CC(C)C)C(=O)N(C)C3(CC3)C(=O)N[C@H](Cc3ccccc3)COc3cnc4ccccc4c3C(=O)N2)c1] | 175 |
| 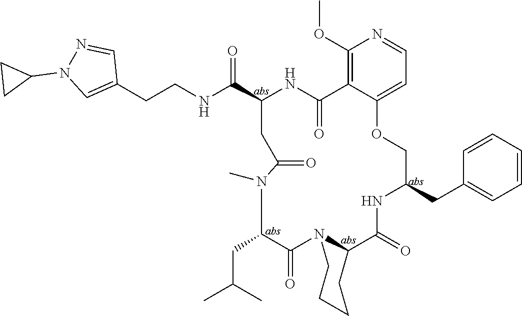 | 176 |
| 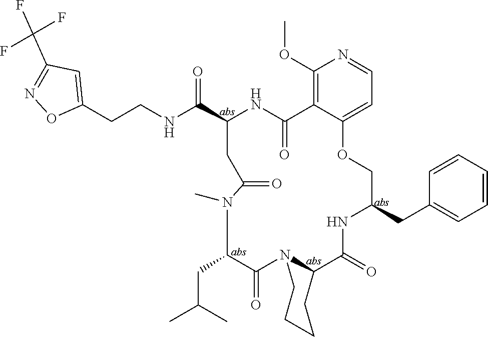 | 177 |
| 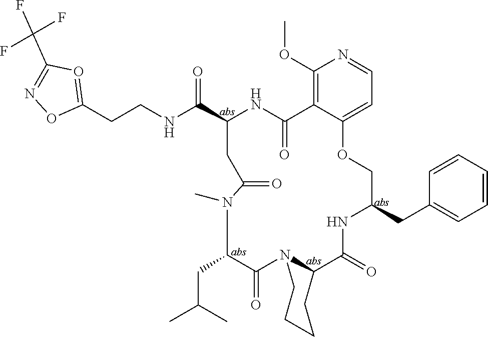 | 178 |
| 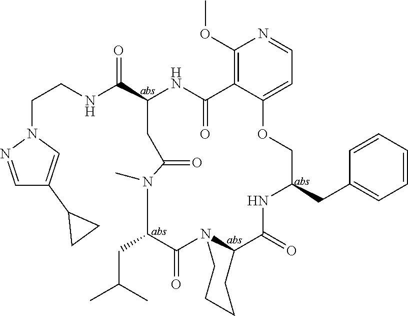 | 179 |
| 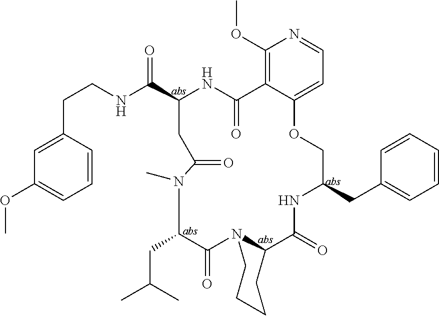 | 180 |
| 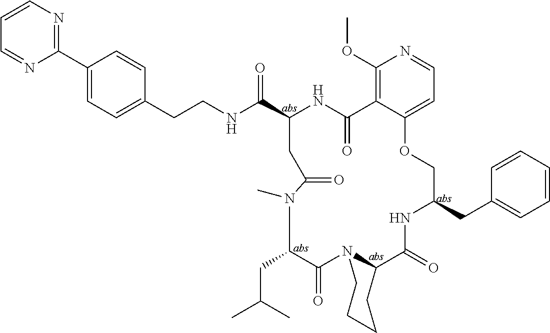 | 181 |
| 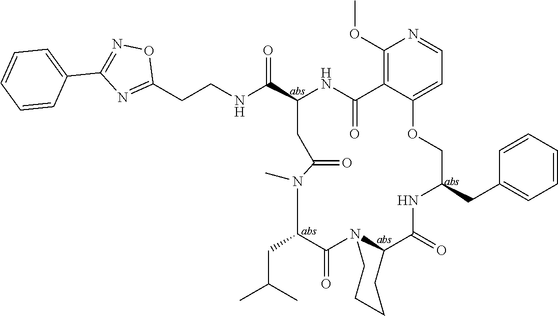 | 182 |
| 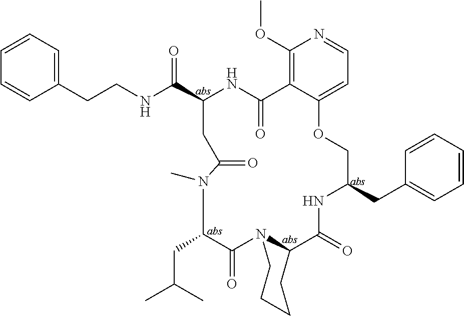 | 183 |
| 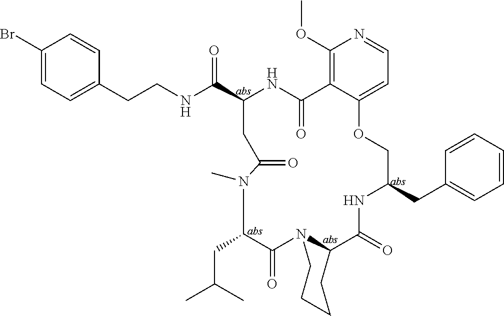 | 184 |
| 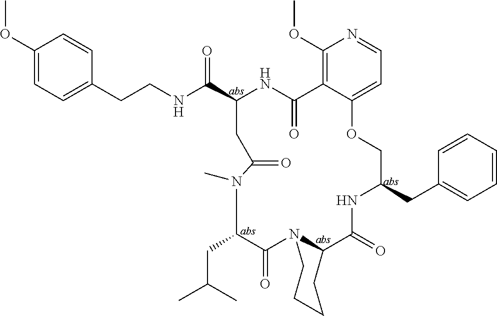 | 185 |
| 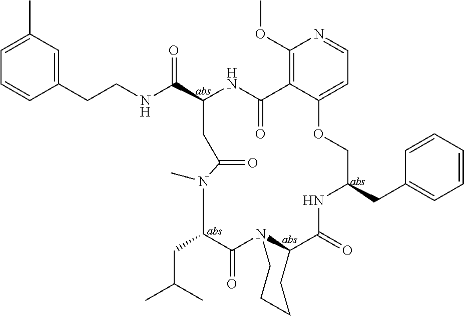 | 186 |
| 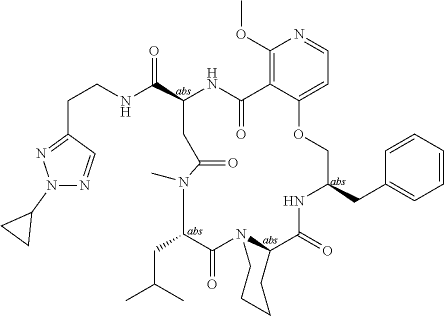 | 187 |
| 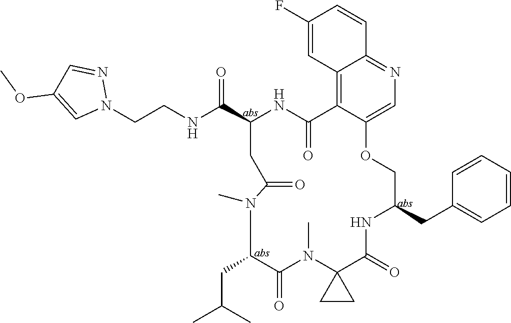 [SMILES: COc1cnn(CCNC(=O)[C@@H]2CC(=O)N(C)[C@@H](CC(C)C)C(=O)N(C)C3(CC3)C(=O)N[C@H](Cc3ccccc3)COc3cnc4ccc(F)cc4c3C(=O)N2)c1] | 188 |
| 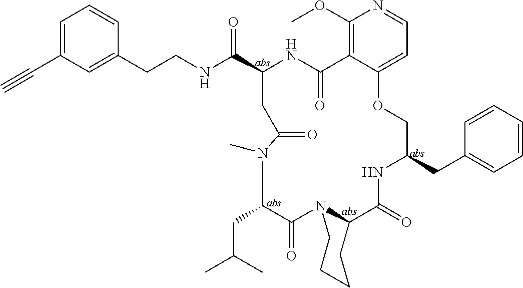 | 189 |
| 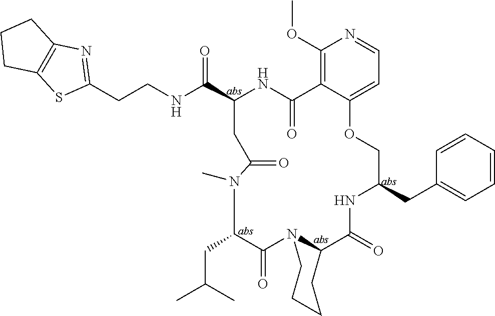 | 190 |
| 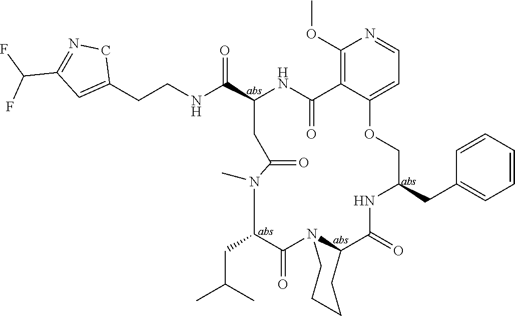 | 191 |
| 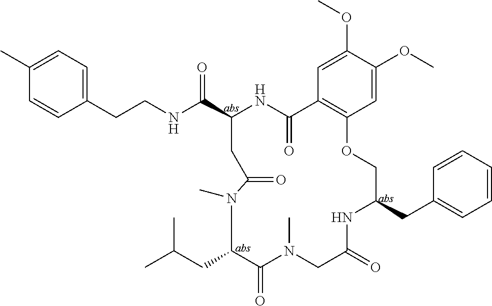 [SMILES: COc1cc2c(cc1OC)C(=O)N[C@H](C(=O)NCCc1ccc(C)cc1)CC(=O)N(C)[C@@H](CC(C)C)C(=O)N(C)CC(=O)N[C@H](Cc1ccccc1)CO2] | 192 |
| 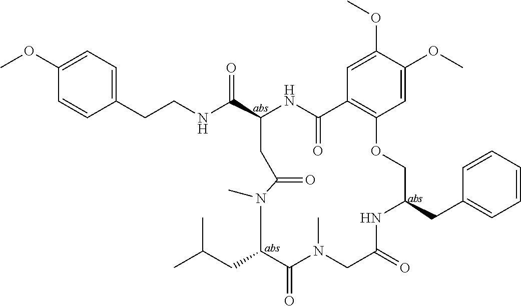 [SMILES: COc1ccc(CCNC(=O)[C@@H]2CC(=O)N(C)[C@@H](CC(C)C)C(=O)N(C)CC(=O)N[C@H](Cc3ccccc3)COc3cc(OC)c(OC)cc3C(=O)N2)cc1] | 193 |
| 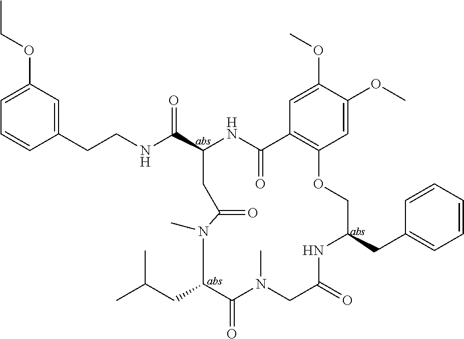 [SMILES: CCOc1cccc(CCNC(=O)[C@@H]2CC(=O)N(C)[C@@H](CC(C)C)C(=O)N(C)CC(=O)N[C@H](Cc3ccccc3)COc3cc(OC)c(OC)cc3C(=O)N2)c1] | 194 |
| 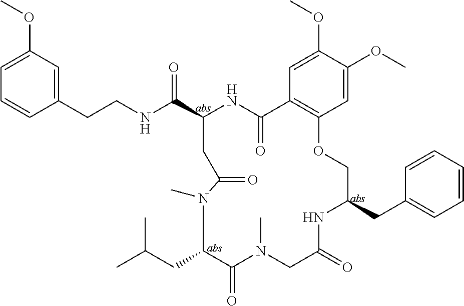 [SMILES: COc1cccc(CCNC(=O)[C@@H]2CC(=O)N(C)[C@@H](CC(C)C)C(=O)N(C)CC(=O)N[C@H](Cc3ccccc3)COc3cc(OC)c(OC)cc3C(=O)N2)c1] | 195 |
| 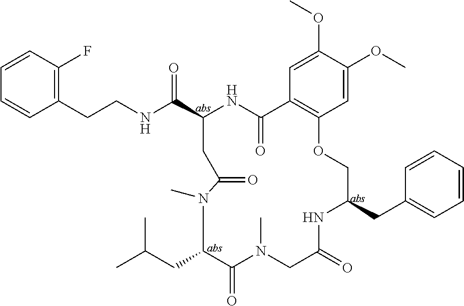 [SMILES: COc1cc2c(cc1OC)C(=O)N[C@H](C(=O)NCCc1ccccc1F)CC(=O)N(C)[C@@H](CC(C)C)C(=O)N(C)CC(=O)N[C@H](Cc1ccccc1)CO2] | 196 |
| 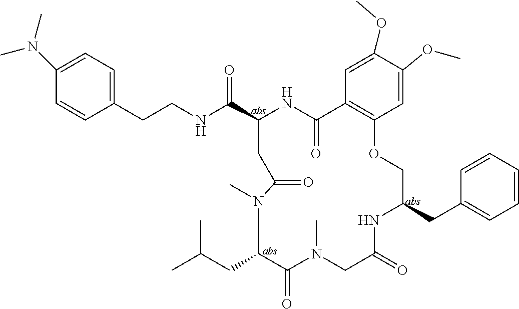 [SMILES: COc1cc2c(cc1OC)C(=O)N[C@H](C(=O)NCCc1ccc(N(C)C)cc1)CC(=O)N(C)[C@@H](CC(C)C)C(=O)N(C)CC(=O)N[C@H](Cc1ccccc1)CO2] | 197 |
| 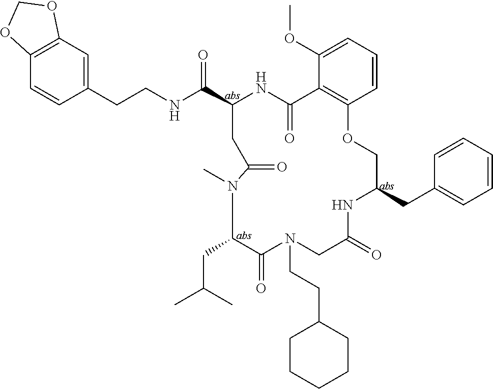 [SMILES: COc1cccc2c1C(=O)N[C@H](C(=O)NCCc1ccc3c(c1)OCO3)CC(=O)N(C)[C@@H](CC(C)C)C(=O)N(CCC1CCCCC1)CC(=O)N[C@H](Cc1ccccc1)CO2] | 198 |
| 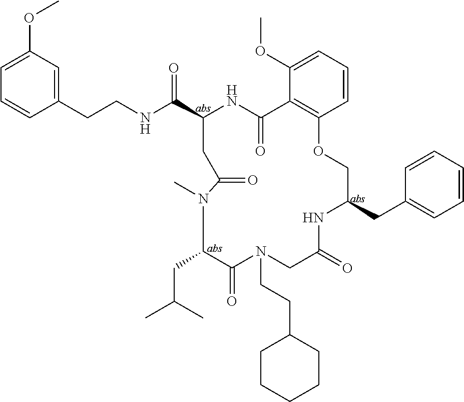 [SMILES: COc1cccc(CCNC(=O)[C@@H]2CC(=O)N(C)[C@@H](CC(C)C)C(=O)N(CCC3CCCCC3)CC(=O)N[C@H](Cc3ccccc3)COc3cccc(OC)c3C(=O)N2)c1] | 199 |
| 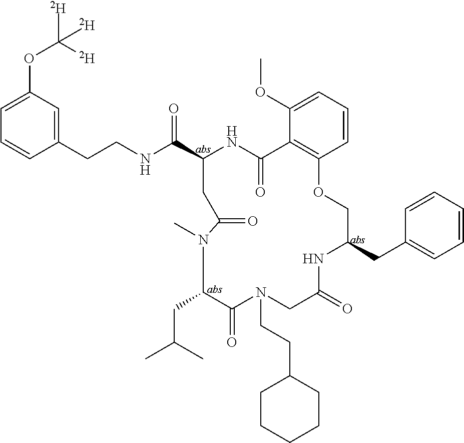 [SMILES: [2H]C([2H])([2H])Oc1cccc(CCNC(=O)[C@@H]2CC(=O)N(C)[C@@H](CC(C)C)C(=O)N(CCC3CCCCC3)CC(=O)N[C@H](Cc3ccccc3)COc3cccc(OC)c3C(=O)N2)c1] | 200 |
| 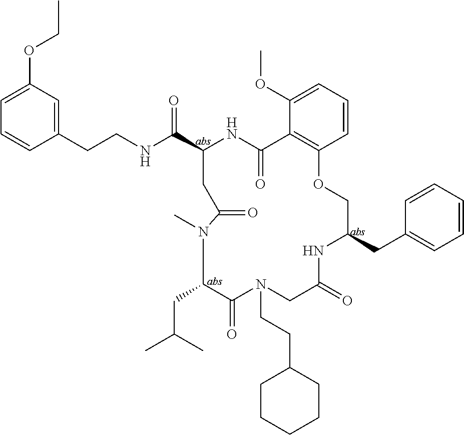 [SMILES: CCOc1cccc(CCNC(=O)[C@@H]2CC(=O)N(C)[C@@H](CC(C)C)C(=O)N(CCC3CCCCC3)CC(=O)N[C@H](Cc3ccccc3)COc3cccc(OC)c3C(=O)N2)c1] | 201 |
| 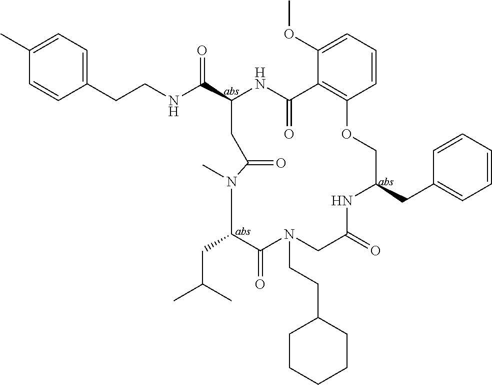 [SMILES: COc1cccc2c1C(=O)N[C@H](C(=O)NCCc1ccc(C)cc1)CC(=O)N(C)[C@@H](CC(C)C)C(=O)N(CCC1CCCCC1)CC(=O)N[C@H](Cc1ccccc1)CO2] | 202 |
| 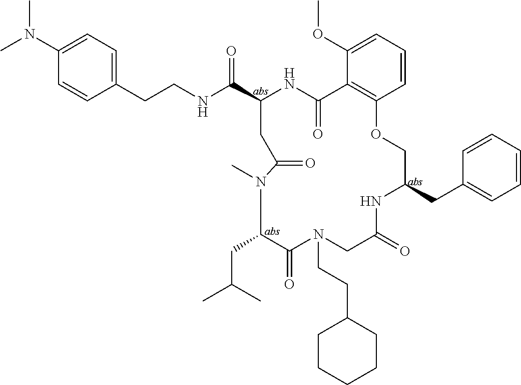 [SMILES: COc1cccc2c1C(=O)N[C@H](C(=O)NCCc1ccc(N(C)C)cc1)CC(=O)N(C)[C@@H](CC(C)C)C(=O)N(CCC1CCCCC1)CC(=O)N[C@H](Cc1ccccc1)CO2] | 203 |
| 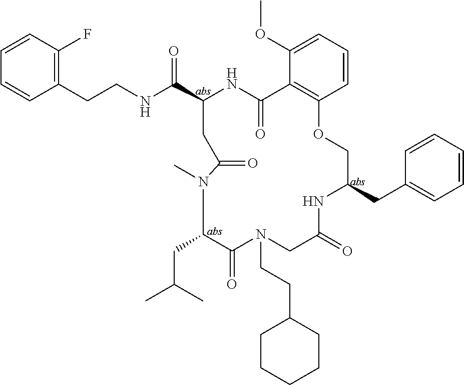 [SMILES: COc1cccc2c1C(=O)N[C@H](C(=O)NCCc1ccccc1F)CC(=O)N(C)[C@@H](CC(C)C)C(=O)N(CCC1CCCCC1)CC(=O)N[C@H](Cc1ccccc1)CO2] | 204 |
| 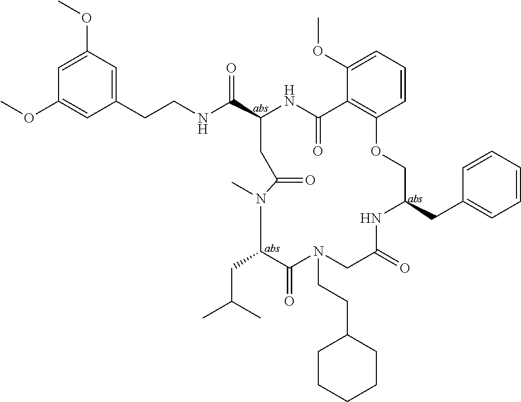 [SMILES: COc1cc(CCNC(=O)[C@@H]2CC(=O)N(C)[C@@H](CC(C)C)C(=O)N(CCC3CCCCC3)CC(=O)N[C@H](Cc3ccccc3)COc3cccc(OC)c3C(=O)N2)cc(OC)c1] | 205 |
| 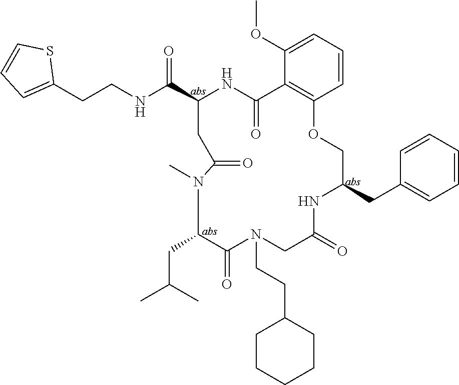 [SMILES: COc1cccc2c1C(=O)N[C@H](C(=O)NCCc1cccs1)CC(=O)N(C)[C@@H](CC(C)C)C(=O)N(CCC1CCCCC1)CC(=O)N[C@H](Cc1ccccc1)CO2] | 206 |
| 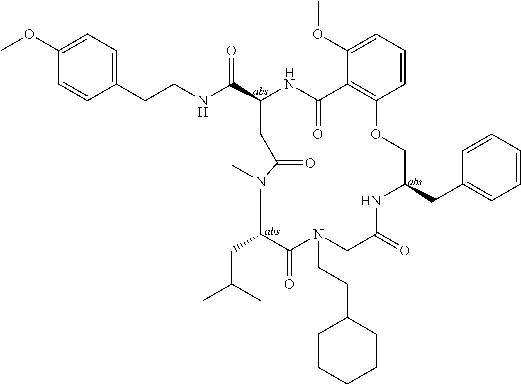 [SMILES: COc1ccc(CCNC(=O)[C@@H]2CC(=O)N(C)[C@@H](CC(C)C)C(=O)N(CCC3CCCCC3)CC(=O)N[C@H](Cc3ccccc3)COc3cccc(OC)c3C(=O)N2)cc1] | 207 |
| 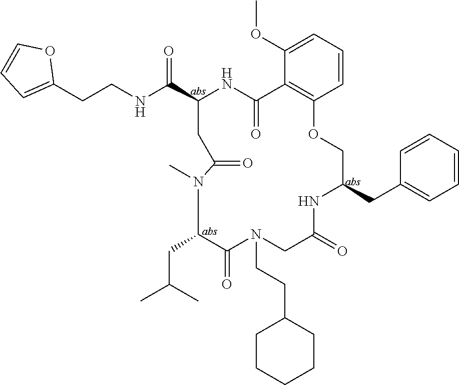 [SMILES: COc1cccc2c1C(=O)N[C@H](C(=O)NCCc1ccco1)CC(=O)N(C)[C@@H](CC(C)C)C(=O)N(CCC1CCCCC1)CC(=O)N[C@H](Cc1ccccc1)CO2] | 208 |
| 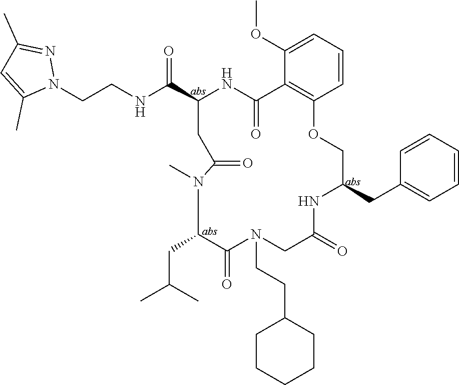 [SMILES: COc1cccc2c1C(=O)N[C@H](C(=O)NCCn1nc(C)cc1C)CC(=O)N(C)[C@@H](CC(C)C)C(=O)N(CCC1CCCCC1)CC(=O)N[C@H](Cc1ccccc1)CO2] | 209 |
| 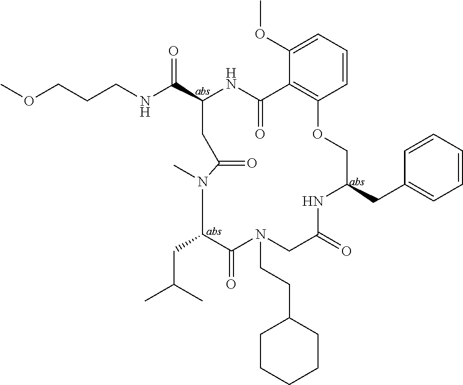 [SMILES: COCCCNC(=O)[C@@H]1CC(=O)N(C)[C@@H](CC(C)C)C(=O)N(CCC2CCCCC2)CC(=O)N[C@H](Cc2ccccc2)COc2cccc(OC)c2C(=O)N1] | 210 |
| 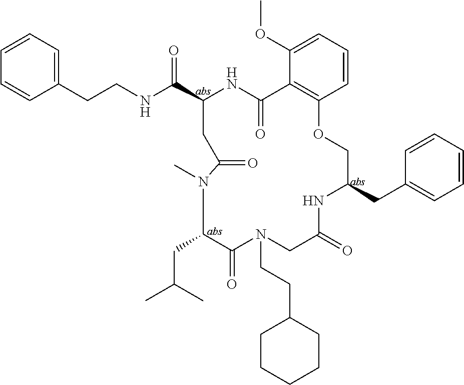 [SMILES: COc1cccc2c1C(=O)N[C@H](C(=O)NCCc1ccccc1)CC(=O)N(C)[C@@H](CC(C)C)C(=O)N(CCC1CCCCC1)CC(=O)N[C@H](Cc1ccccc1)CO2] | 211 |
| 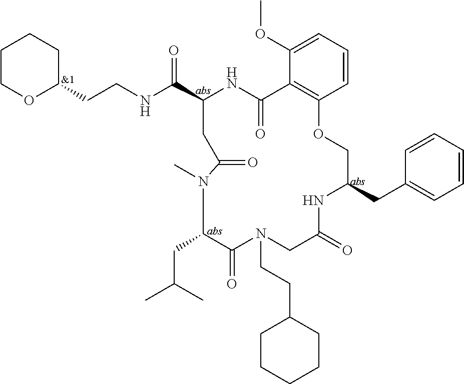 [SMILES: COc1cccc2c1C(=O)N[C@H](C(=O)NCC[C@H]1CCCCO1)CC(=O)N(C)[C@@H](CC(C)C)C(=O)N(CCC1CCCCC1)CC(=O)N[C@H](Cc1ccccc1)CO2] | 212 |
| 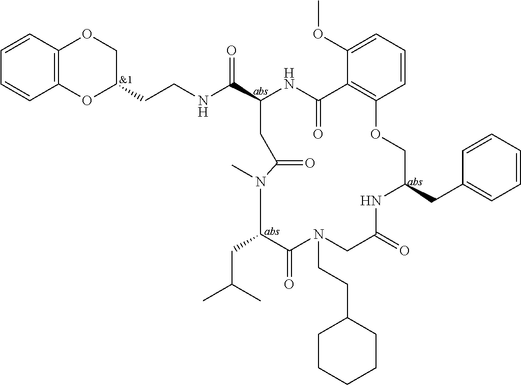 [SMILES: COc1cccc2c1C(=O)N[C@H](C(=O)NCC[C@H]1COc3ccccc3O1)CC(=O)N(C)[C@@H](CC(C)C)C(=O)N(CCC1CCCCC1)CC(=O)N[C@H](Cc1ccccc1)CO2] | 213 |
| 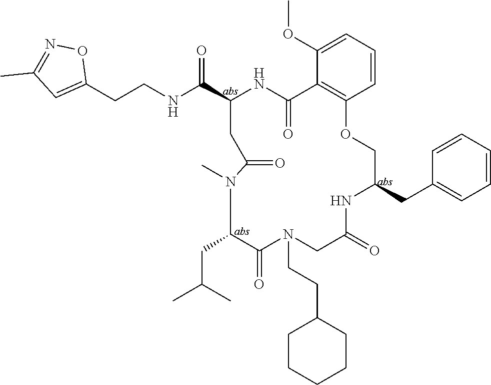 [SMILES: COc1cccc2c1C(=O)N[C@H](C(=O)NCCc1cc(C)no1)CC(=O)N(C)[C@@H](CC(C)C)C(=O)N(CCC1CCCCC1)CC(=O)N[C@H](Cc1ccccc1)CO2] | 214 |
| 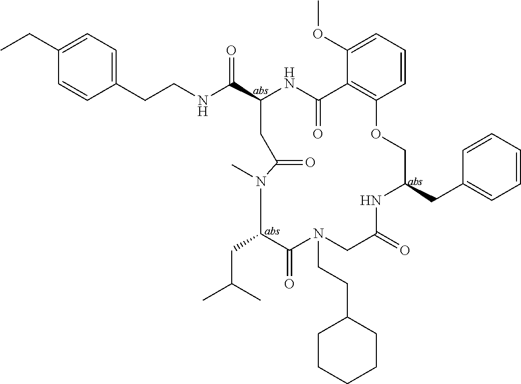 [SMILES: CCc1ccc(CCNC(=O)[C@@H]2CC(=O)N(C)[C@@H](CC(C)C)C(=O)N(CCC3CCCCC3)CC(=O)N[C@H](Cc3ccccc3)COc3cccc(OC)c3C(=O)N2)cc1] | 215 |
| 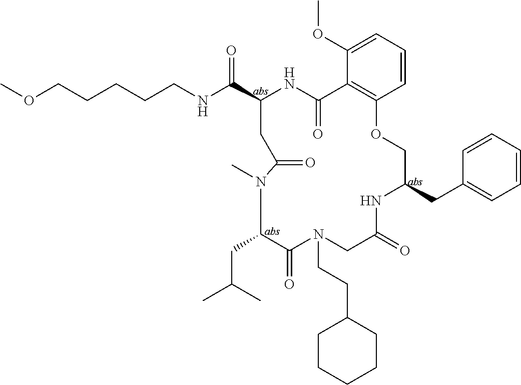 [SMILES: COCCCCCNC(=O)[C@@H]1CC(=O)N(C)[C@@H](CC(C)C)C(=O)N(CCC2CCCCC2)CC(=O)N[C@H](Cc2ccccc2)COc2cccc(OC)c2C(=O)N1] | 216 |
| 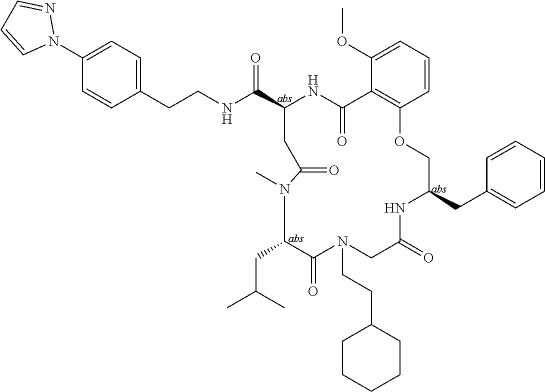 [SMILES: COc1cccc2c1C(=O)N[C@H](C(=O)NCCc1ccc(-n3cccn3)cc1)CC(=O)N(C)[C@@H](CC(C)C)C(=O)N(CCC1CCCCC1)CC(=O)N[C@H](Cc1ccccc1)CO2] | 217 |
| 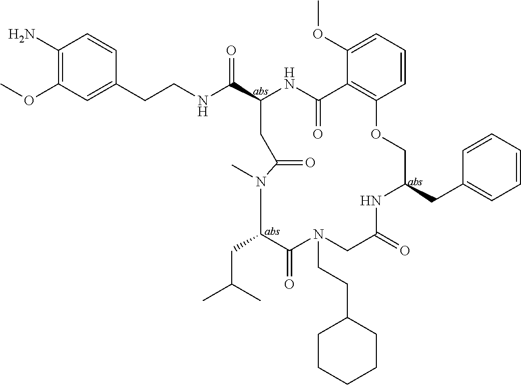 [SMILES: COc1cc(CCNC(=O)[C@@H]2CC(=O)N(C)[C@@H](CC(C)C)C(=O)N(CCC3CCCCC3)CC(=O)N[C@H](Cc3ccccc3)COc3cccc(OC)c3C(=O)N2)ccc1N] | 218 |
| 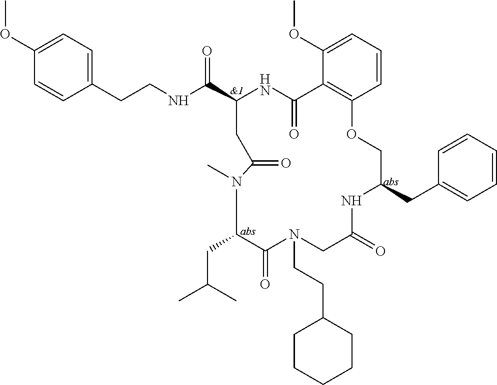 [SMILES: COc1ccc(CCNC(=O)[C@@H]2CC(=O)N(C)[C@@H](CC(C)C)C(=O)N(CCC3CCCCC3)CC(=O)N[C@H](Cc3ccccc3)COc3cccc(OC)c3C(=O)N2)cc1] | 219 |
| 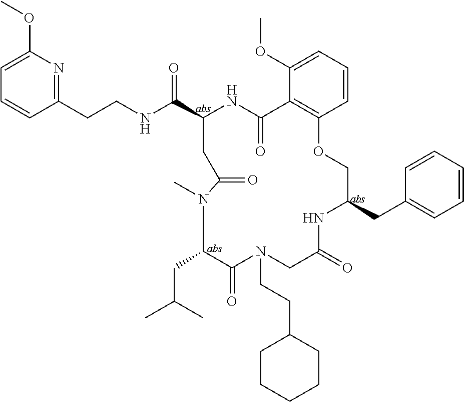 [SMILES: COc1cccc(CCNC(=O)[C@@H]2CC(=O)N(C)[C@@H](CC(C)C)C(=O)N(CCC3CCCCC3)CC(=O)N[C@H](Cc3ccccc3)COc3cccc(OC)c3C(=O)N2)n1] | 220 |
| 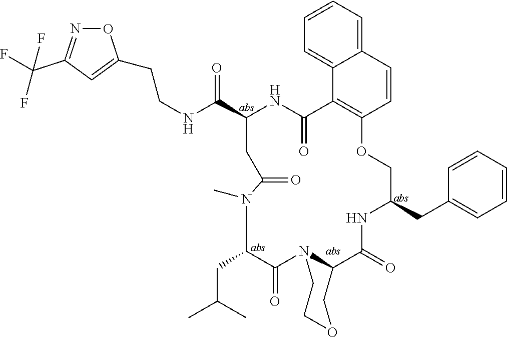 | 221 |
| 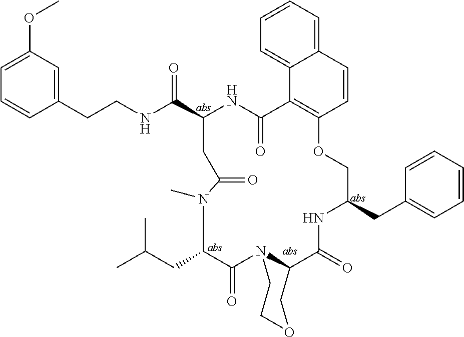 | 222 |
| 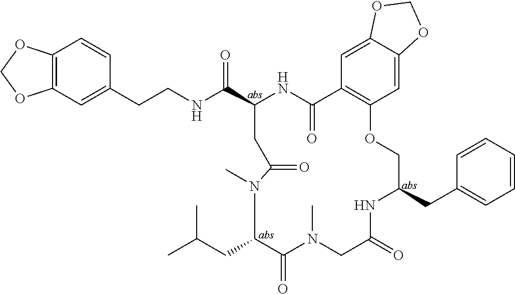 [SMILES: CC(C)C[C@H]1C(=O)N(C)CC(=O)N[C@H](Cc2ccccc2)COc2cc3c(cc2C(=O)N[C@H](C(=O)NCCc2ccc4c(c2)OCO4)CC(=O)N1C)OCO3] | 223 |
| 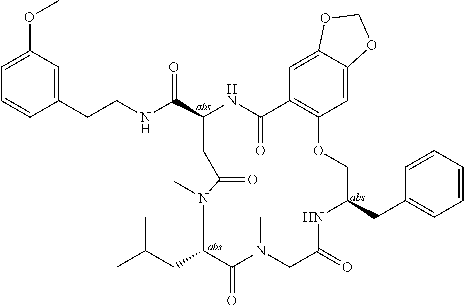 [SMILES: COc1cccc(CCNC(=O)[C@@H]2CC(=O)N(C)[C@@H](CC(C)C)C(=O)N(C)CC(=O)N[C@H](Cc3ccccc3)COc3cc4c(cc3C(=O)N2)OCO4)c1] | 224 |
| 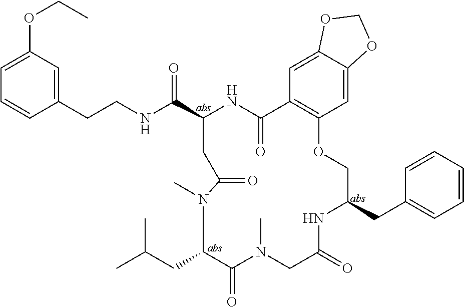 [SMILES: CCOc1cccc(CCNC(=O)[C@@H]2CC(=O)N(C)[C@@H](CC(C)C)C(=O)N(C)CC(=O)N[C@H](Cc3ccccc3)COc3cc4c(cc3C(=O)N2)OCO4)c1] | 225 |
| 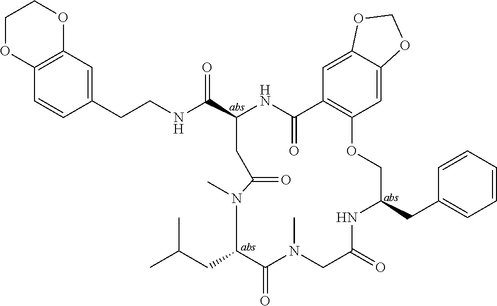 [SMILES: CC(C)C[C@H]1C(=O)N(C)CC(=O)N[C@H](Cc2ccccc2)COc2cc3c(cc2C(=O)N[C@H](C(=O)NCCc2ccc4c(c2)OCCO4)CC(=O)N1C)OCO3] | 226 |
| 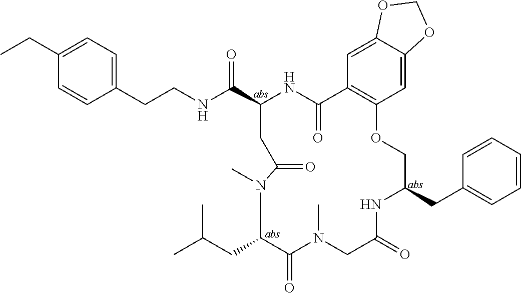 [SMILES: CCc1ccc(CCNC(=O)[C@@H]2CC(=O)N(C)[C@@H](CC(C)C)C(=O)N(C)CC(=O)N[C@H](Cc3ccccc3)COc3cc4c(cc3C(=O)N2)OCO4)cc1] | 227 |
| 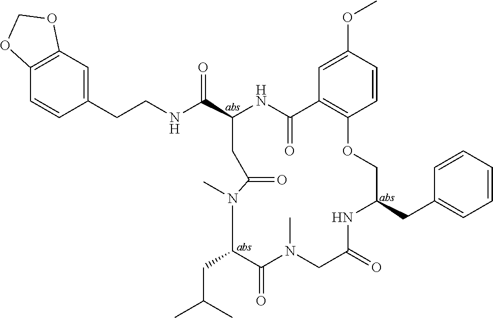 [SMILES: COc1ccc2c(c1)C(=O)N[C@H](C(=O)NCCc1ccc3c(c1)OCO3)CC(=O)N(C)[C@@H](CC(C)C)C(=O)N(C)CC(=O)N[C@H](Cc1ccccc1)CO2] | 228 |
| 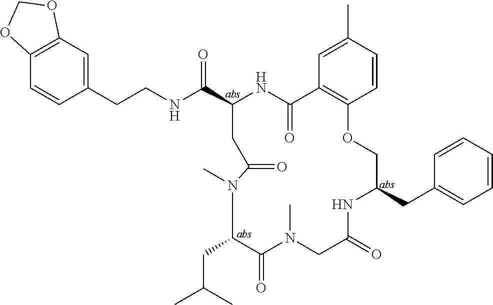 [SMILES: Cc1ccc2c(c1)C(=O)N[C@H](C(=O)NCCc1ccc3c(c1)OCO3)CC(=O)N(C)[C@@H](CC(C)C)C(=O)N(C)CC(=O)N[C@H](Cc1ccccc1)CO2] | 229 |
| 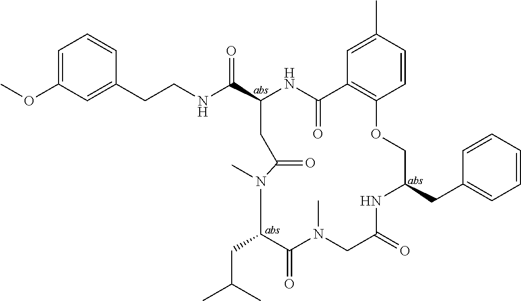 [SMILES: COc1cccc(CCNC(=O)[C@@H]2CC(=O)N(C)[C@@H](CC(C)C)C(=O)N(C)CC(=O)N[C@H](Cc3ccccc3)COc3ccc(C)cc3C(=O)N2)c1] | 230 |
| 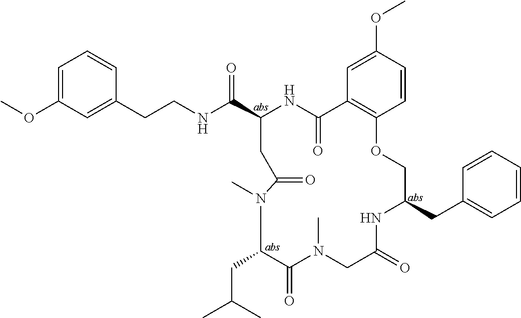 [SMILES: COc1cccc(CCNC(=O)[C@@H]2CC(=O)N(C)[C@@H](CC(C)C)C(=O)N(C)CC(=O)N[C@H](Cc3ccccc3)COc3ccc(OC)cc3C(=O)N2)c1] | 231 |
| 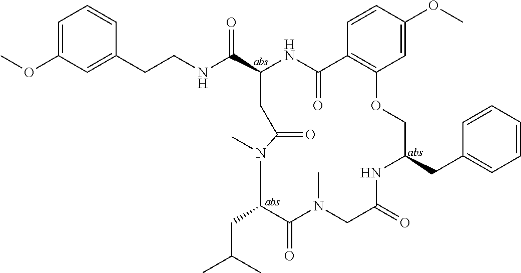 [SMILES: COc1cccc(CCNC(=O)[C@@H]2CC(=O)N(C)[C@@H](CC(C)C)C(=O)N(C)CC(=O)N[C@H](Cc3ccccc3)COc3cc(OC)ccc3C(=O)N2)c1] | 232 |
| 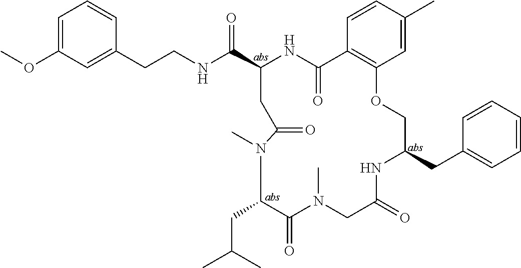 [SMILES: COc1cccc(CCNC(=O)[C@@H]2CC(=O)N(C)[C@@H](CC(C)C)C(=O)N(C)CC(=O)N[C@H](Cc3ccccc3)COc3cc(C)ccc3C(=O)N2)c1] | 233 |
| 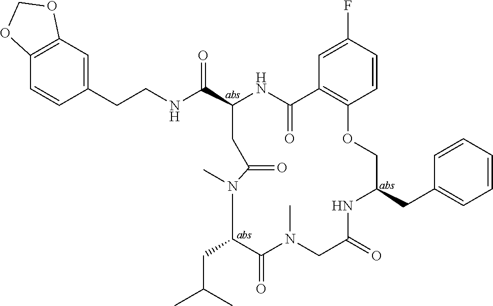 [SMILES: CC(C)C[C@H]1C(=O)N(C)CC(=O)N[C@H](Cc2ccccc2)COc2ccc(F)cc2C(=O)N[C@H](C(=O)NCCc2ccc3c(c2)OCO3)CC(=O)N1C] | 234 |
| 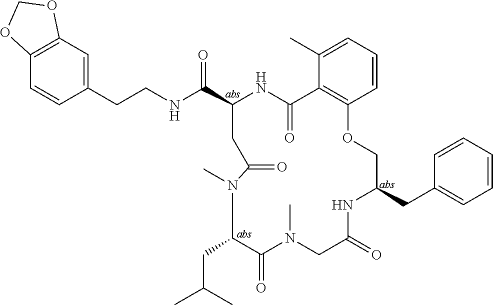 [SMILES: Cc1cccc2c1C(=O)N[C@H](C(=O)NCCc1ccc3c(c1)OCO3)CC(=O)N(C)[C@@H](CC(C)C)C(=O)N(C)CC(=O)N[C@H](Cc1ccccc1)CO2] | 235 |
| 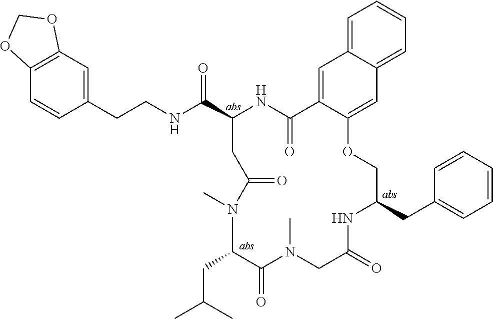 [SMILES: CC(C)C[C@H]1C(=O)N(C)CC(=O)N[C@H](Cc2ccccc2)COc2cc3ccccc3cc2C(=O)N[C@H](C(=O)NCCc2ccc3c(c2)OCO3)CC(=O)N1C] | 236 |
| 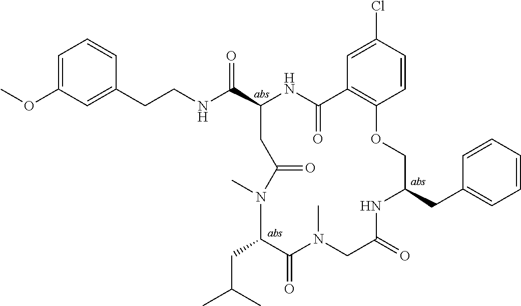 [SMILES: COc1cccc(CCNC(=O)[C@@H]2CC(=O)N(C)[C@@H](CC(C)C)C(=O)N(C)CC(=O)N[C@H](Cc3ccccc3)COc3ccc(Cl)cc3C(=O)N2)c1] | 237 |
| 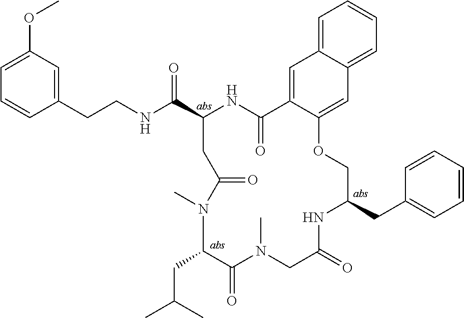 [SMILES: COc1cccc(CCNC(=O)[C@@H]2CC(=O)N(C)[C@@H](CC(C)C)C(=O)N(C)CC(=O)N[C@H](Cc3ccccc3)COc3cc4ccccc4cc3C(=O)N2)c1] | 238 |
| 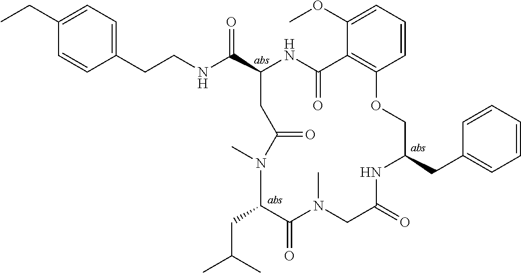 [SMILES: CCc1ccc(CCNC(=O)[C@@H]2CC(=O)N(C)[C@@H](CC(C)C)C(=O)N(C)CC(=O)N[C@H](Cc3ccccc3)COc3cccc(OC)c3C(=O)N2)cc1] | 239 |
| 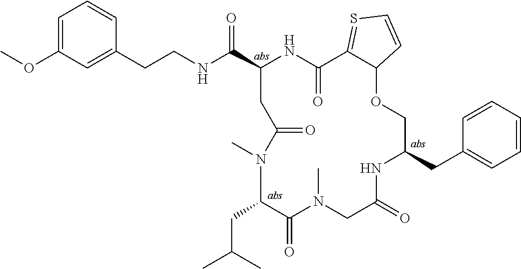 | 240 |
| 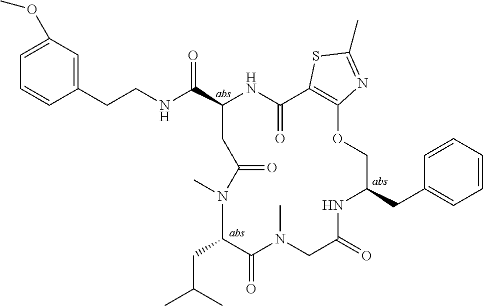 [SMILES: COc1cccc(CCNC(=O)[C@@H]2CC(=O)N(C)[C@@H](CC(C)C)C(=O)N(C)CC(=O)N[C@H](Cc3ccccc3)COc3nc(C)sc3C(=O)N2)c1] | 241 |
| 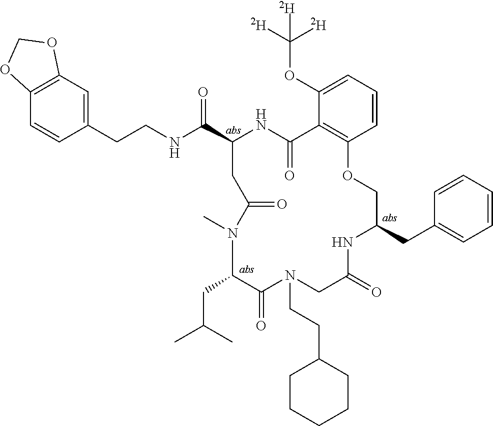 [SMILES: [2H]C([2H])([2H])Oc1cccc2c1C(=O)N[C@H](C(=O)NCCc1ccc3c(c1)OCO3)CC(=O)N(C)[C@@H](CC(C)C)C(=O)N(CCC1CCCCC1)CC(=O)N[C@H](Cc1ccccc1)CO2] | 242 |
| 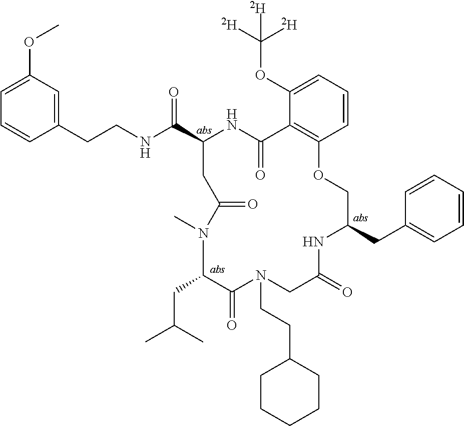 [SMILES: [2H]C([2H])([2H])Oc1cccc2c1C(=O)N[C@H](C(=O)NCCc1cccc(OC)c1)CC(=O)N(C)[C@@H](CC(C)C)C(=O)N(CCC1CCCCC1)CC(=O)N[C@H](Cc1ccccc1)CO2] | 243 |
| 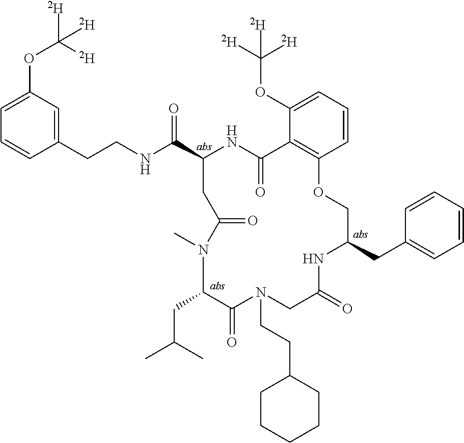 [SMILES: [2H]C([2H])([2H])Oc1cccc(CCNC(=O)[C@@H]2CC(=O)N(C)[C@@H](CC(C)C)C(=O)N(CCC3CCCCC3)CC(=O)N[C@H](Cc3ccccc3)COc3cccc(OC([2H])([2H])[2H])c3C(=O)N2)c1] | 244 |
| 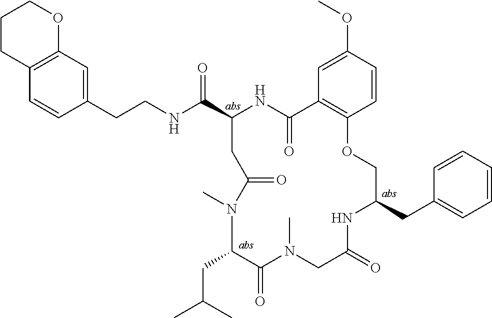 [SMILES: COc1ccc2c(c1)C(=O)N[C@H](C(=O)NCCc1ccc3c(c1)OCCC3)CC(=O)N(C)[C@@H](CC(C)C)C(=O)N(C)CC(=O)N[C@H](Cc1ccccc1)CO2] | 245 |
| 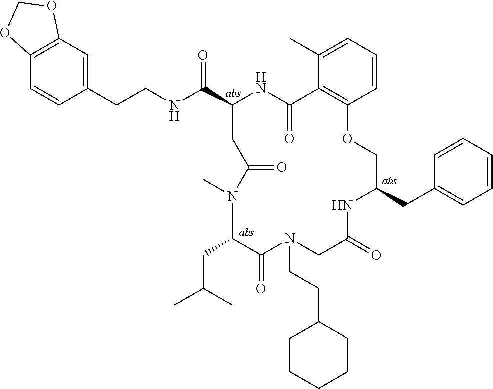 [SMILES: Cc1cccc2c1C(=O)N[C@H](C(=O)NCCc1ccc3c(c1)OCO3)CC(=O)N(C)[C@@H](CC(C)C)C(=O)N(CCC1CCCCC1)CC(=O)N[C@H](Cc1ccccc1)CO2] | 246 |
| 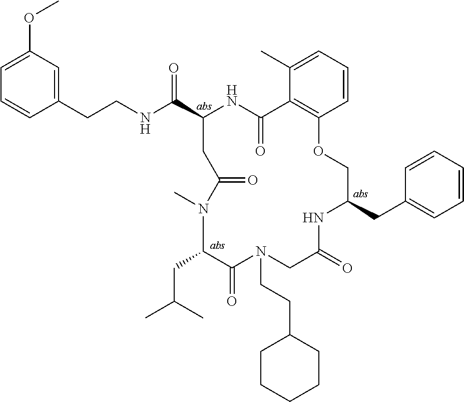 [SMILES: COc1cccc(CCNC(=O)[C@@H]2CC(=O)N(C)[C@@H](CC(C)C)C(=O)N(CCC3CCCCC3)CC(=O)N[C@H](Cc3ccccc3)COc3cccc(C)c3C(=O)N2)c1] | 247 |
| 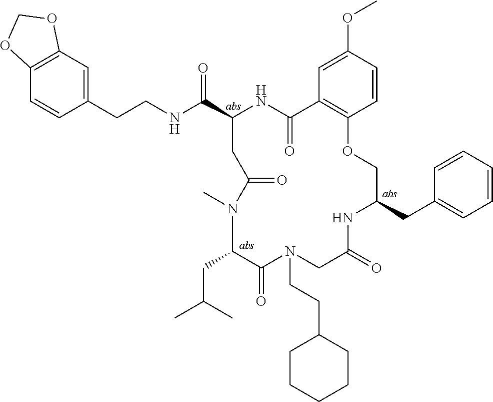 [SMILES: COc1ccc2c(c1)C(=O)N[C@H](C(=O)NCCc1ccc3c(c1)OCO3)CC(=O)N(C)[C@@H](CC(C)C)C(=O)N(CCC1CCCCC1)CC(=O)N[C@H](Cc1ccccc1)CO2] | 248 |
| 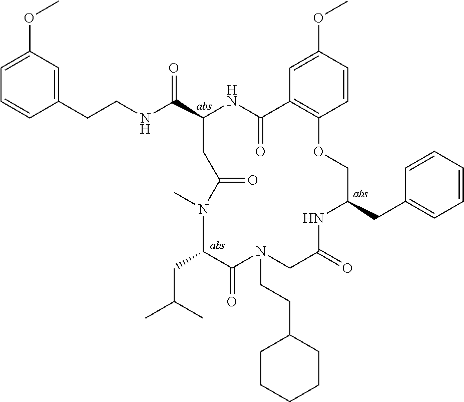 [SMILES: COc1cccc(CCNC(=O)[C@@H]2CC(=O)N(C)[C@@H](CC(C)C)C(=O)N(CCC3CCCCC3)CC(=O)N[C@H](Cc3ccccc3)COc3ccc(OC)cc3C(=O)N2)c1] | 249 |
| 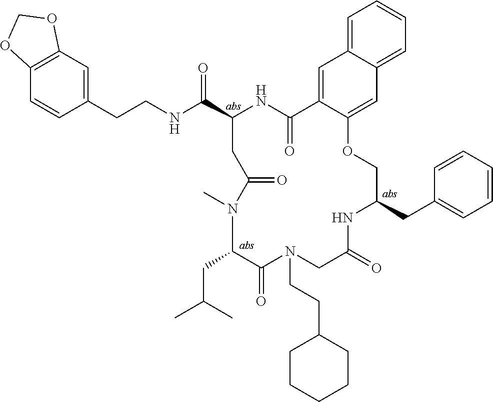 [SMILES: CC(C)C[C@H]1C(=O)N(CCC2CCCCC2)CC(=O)N[C@H](Cc2ccccc2)COc2cc3ccccc3cc2C(=O)N[C@H](C(=O)NCCc2ccc3c(c2)OCO3)CC(=O)N1C] | 250 |
| 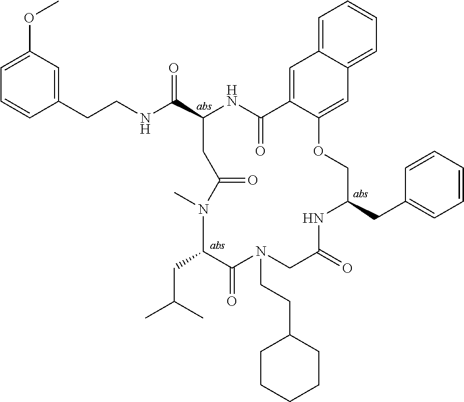 [SMILES: COc1cccc(CCNC(=O)[C@@H]2CC(=O)N(C)[C@@H](CC(C)C)C(=O)N(CCC3CCCCC3)CC(=O)N[C@H](Cc3ccccc3)COc3cc4ccccc4cc3C(=O)N2)c1] | 251 |
| 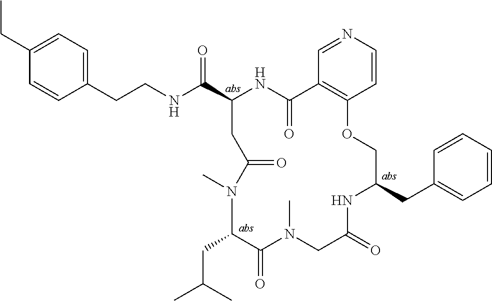 [SMILES: CCc1ccc(CCNC(=O)[C@@H]2CC(=O)N(C)[C@@H](CC(C)C)C(=O)N(C)CC(=O)N[C@H](Cc3ccccc3)COc3ccncc3C(=O)N2)cc1] | 252 |
| 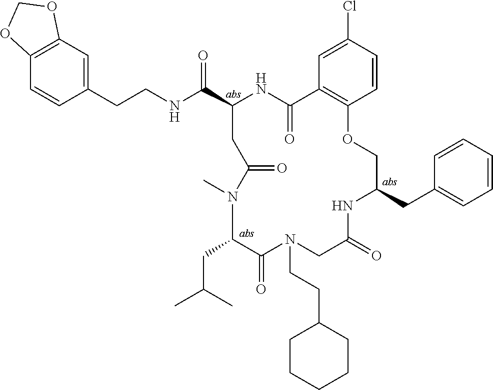 [SMILES: CC(C)C[C@H]1C(=O)N(CCC2CCCCC2)CC(=O)N[C@H](Cc2ccccc2)COc2ccc(Cl)cc2C(=O)N[C@H](C(=O)NCCc2ccc3c(c2)OCO3)CC(=O)N1C] | 253 |
| 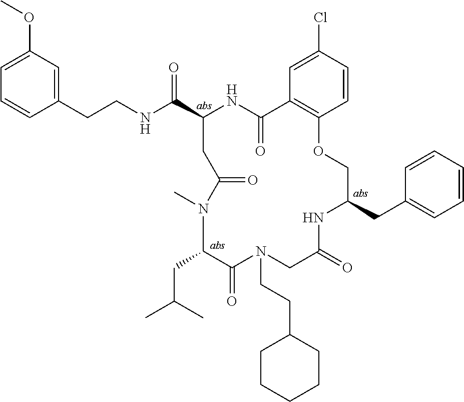 [SMILES: COc1cccc(CCNC(=O)[C@@H]2CC(=O)N(C)[C@@H](CC(C)C)C(=O)N(CCC3CCCCC3)CC(=O)N[C@H](Cc3ccccc3)COc3ccc(Cl)cc3C(=O)N2)c1] | 254 |
| 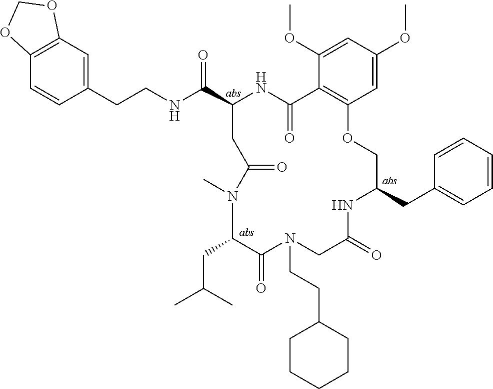 [SMILES: COc1cc(OC)c2c(c1)OC[C@@H](Cc1ccccc1)NC(=O)CN(CCC1CCCCC1)C(=O)[C@H](CC(C)C)N(C)C(=O)C[C@@H](C(=O)NCCc1ccc3c(c1)OCO3)NC2=O] | 255 |
| 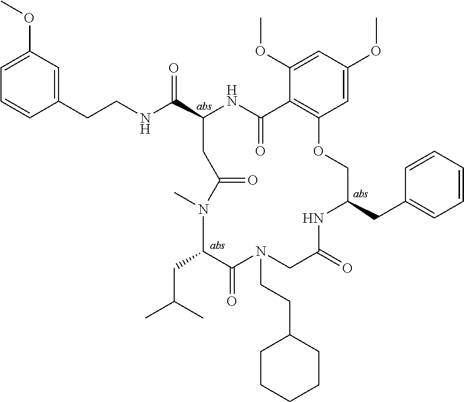 [SMILES: COc1cccc(CCNC(=O)[C@@H]2CC(=O)N(C)[C@@H](CC(C)C)C(=O)N(CCC3CCCCC3)CC(=O)N[C@H](Cc3ccccc3)COc3cc(OC)cc(OC)c3C(=O)N2)c1] | 256 |
| 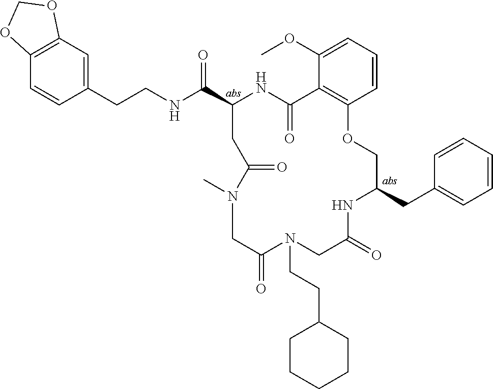 [SMILES: COc1cccc2c1C(=O)N[C@H](C(=O)NCCc1ccc3c(c1)OCO3)CC(=O)N(C)CC(=O)N(CCC1CCCCC1)CC(=O)N[C@H](Cc1ccccc1)CO2] | 257 |
| 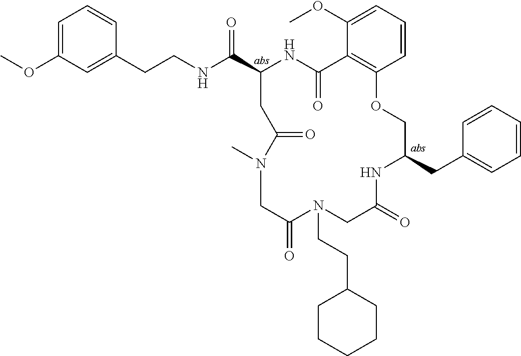 [SMILES: COc1cccc(CCNC(=O)[C@@H]2CC(=O)N(C)CC(=O)N(CCC3CCCCC3)CC(=O)N[C@H](Cc3ccccc3)COc3cccc(OC)c3C(=O)N2)c1] | 258 |
| 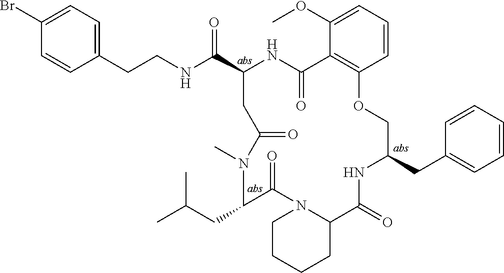 [SMILES: COc1cccc2c1C(=O)N[C@H](C(=O)NCCc1ccc(Br)cc1)CC(=O)N(C)[C@@H](CC(C)C)C(=O)N1CCCCC1C(=O)N[C@H](Cc1ccccc1)CO2] | 259 |
| 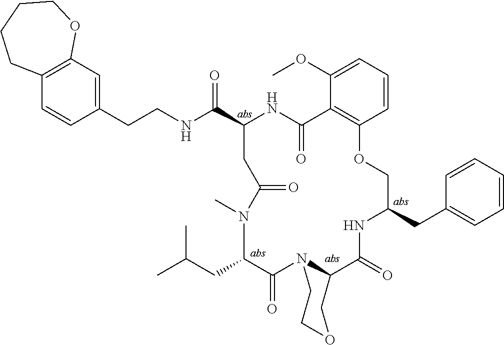 | 260 |
| 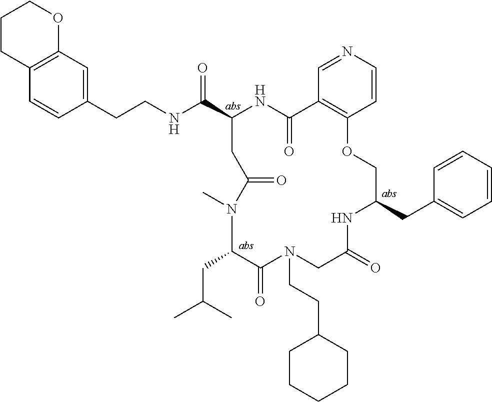 [SMILES: CC(C)C[C@H]1C(=O)N(CCC2CCCCC2)CC(=O)N[C@H](Cc2ccccc2)COc2ccncc2C(=O)N[C@H](C(=O)NCCc2ccc3c(c2)OCCC3)CC(=O)N1C] | 261 |
| 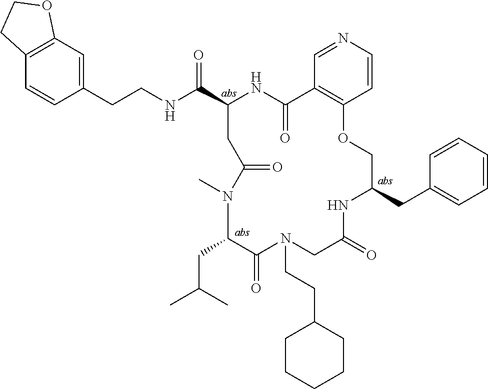 [SMILES: CC(C)C[C@H]1C(=O)N(CCC2CCCCC2)CC(=O)N[C@H](Cc2ccccc2)COc2ccncc2C(=O)N[C@H](C(=O)NCCc2ccc3c(c2)OCC3)CC(=O)N1C] | 262 |
| 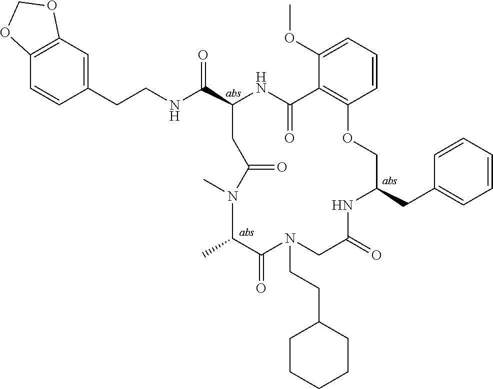 [SMILES: COc1cccc2c1C(=O)N[C@H](C(=O)NCCc1ccc3c(c1)OCO3)CC(=O)N(C)[C@@H](C)C(=O)N(CCC1CCCCC1)CC(=O)N[C@H](Cc1ccccc1)CO2] | 263 |
| 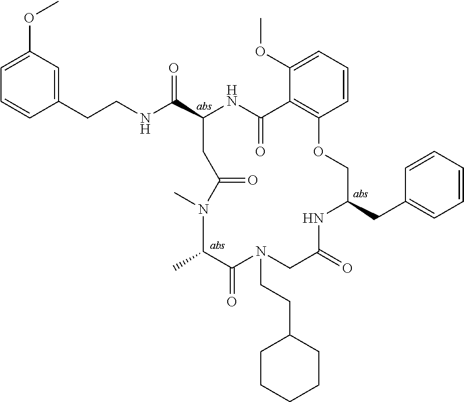 [SMILES: COc1cccc(CCNC(=O)[C@@H]2CC(=O)N(C)[C@@H](C)C(=O)N(CCC3CCCCC3)CC(=O)N[C@H](Cc3ccccc3)COc3cccc(OC)c3C(=O)N2)c1] | 264 |
| 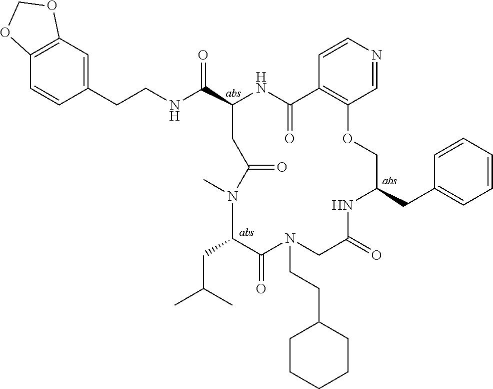 [SMILES: CC(C)C[C@H]1C(=O)N(CCC2CCCCC2)CC(=O)N[C@H](Cc2ccccc2)COc2cnccc2C(=O)N[C@H](C(=O)NCCc2ccc3c(c2)OCO3)CC(=O)N1C] | 265 |
| 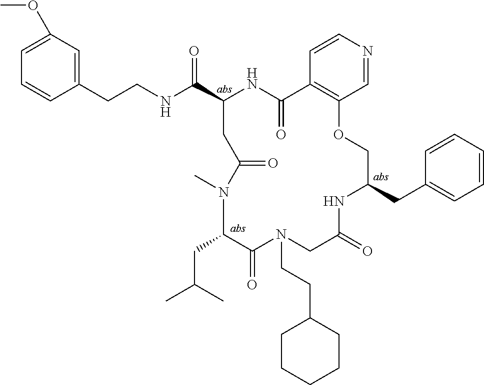 [SMILES: COc1cccc(CCNC(=O)[C@@H]2CC(=O)N(C)[C@@H](CC(C)C)C(=O)N(CCC3CCCCC3)CC(=O)N[C@H](Cc3ccccc3)COc3cnccc3C(=O)N2)c1] | 266 |
| 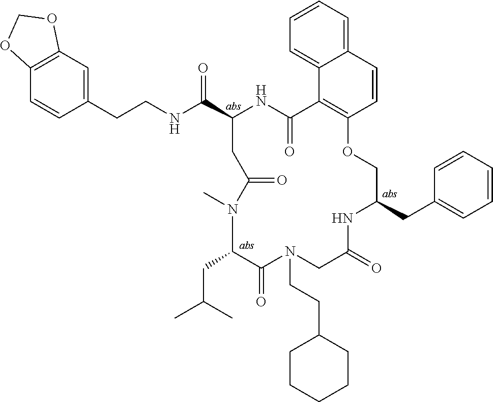 [SMILES: CC(C)C[C@H]1C(=O)N(CCC2CCCCC2)CC(=O)N[C@H](Cc2ccccc2)COc2ccc3ccccc3c2C(=O)N[C@H](C(=O)NCCc2ccc3c(c2)OCO3)CC(=O)N1C] | 267 |
| 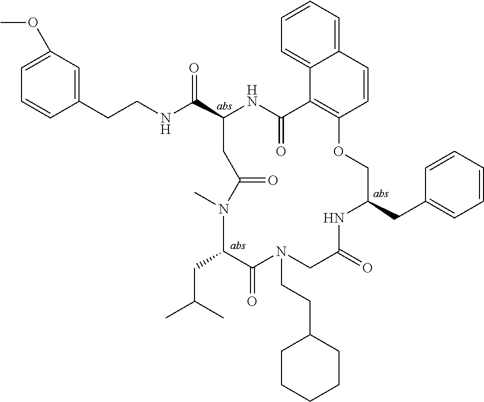 [SMILES: COc1cccc(CCNC(=O)[C@@H]2CC(=O)N(C)[C@@H](CC(C)C)C(=O)N(CCC3CCCCC3)CC(=O)N[C@H](Cc3ccccc3)COc3ccc4ccccc4c3C(=O)N2)c1] | 268 |
| 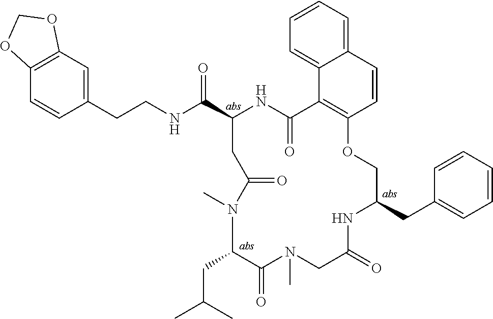 [SMILES: CC(C)C[C@H]1C(=O)N(C)CC(=O)N[C@H](Cc2ccccc2)COc2ccc3ccccc3c2C(=O)N[C@H](C(=O)NCCc2ccc3c(c2)OCO3)CC(=O)N1C] | 269 |
| 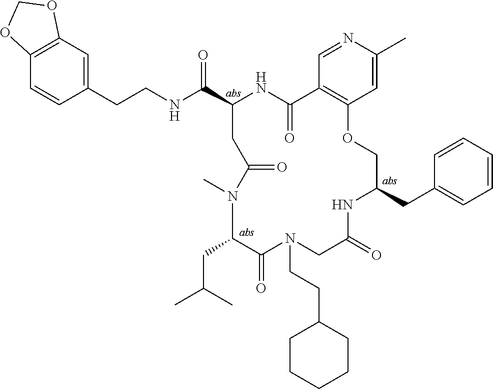 [SMILES: Cc1cc2c(cn1)C(=O)N[C@H](C(=O)NCCc1ccc3c(c1)OCO3)CC(=O)N(C)[C@@H](CC(C)C)C(=O)N(CCC1CCCCC1)CC(=O)N[C@H](Cc1ccccc1)CO2] | 270 |
| 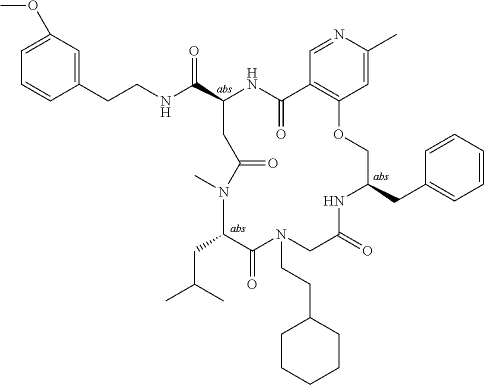 [SMILES: COc1cccc(CCNC(=O)[C@@H]2CC(=O)N(C)[C@@H](CC(C)C)C(=O)N(CCC3CCCCC3)CC(=O)N[C@H](Cc3ccccc3)COc3cc(C)ncc3C(=O)N2)c1] | 271 |
| 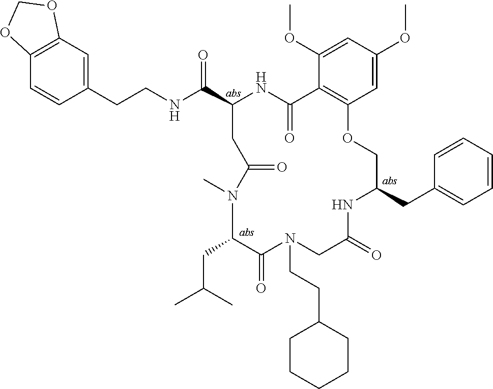 [SMILES: COc1cc(OC)c2c(c1)OC[C@@H](Cc1ccccc1)NC(=O)CN(CCC1CCCCC1)C(=O)[C@H](CC(C)C)N(C)C(=O)C[C@@H](C(=O)NCCc1ccc3c(c1)OCO3)NC2=O] | 272 |
| 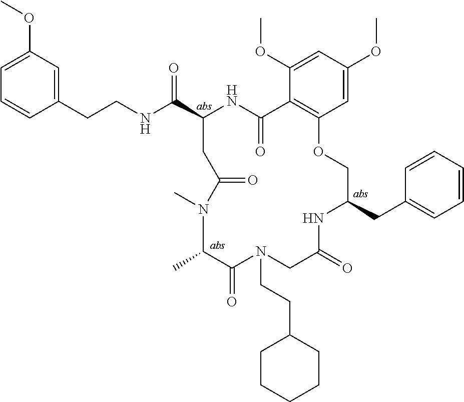 [SMILES: COc1cccc(CCNC(=O)[C@@H]2CC(=O)N(C)[C@@H](C)C(=O)N(CCC3CCCCC3)CC(=O)N[C@H](Cc3ccccc3)COc3cc(OC)cc(OC)c3C(=O)N2)c1] | 273 |
| 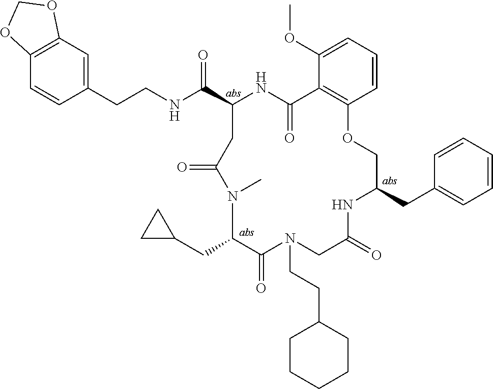 [SMILES: COc1cccc2c1C(=O)N[C@H](C(=O)NCCc1ccc3c(c1)OCO3)CC(=O)N(C)[C@@H](CC1CC1)C(=O)N(CCC1CCCCC1)CC(=O)N[C@H](Cc1ccccc1)CO2] | 274 |
| 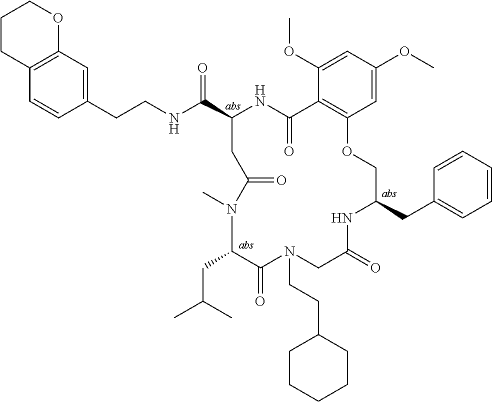 [SMILES: COc1cc(OC)c2c(c1)OC[C@@H](Cc1ccccc1)NC(=O)CN(CCC1CCCCC1)C(=O)[C@H](CC(C)C)N(C)C(=O)C[C@@H](C(=O)NCCc1ccc3c(c1)OCCC3)NC2=O] | 275 |
| 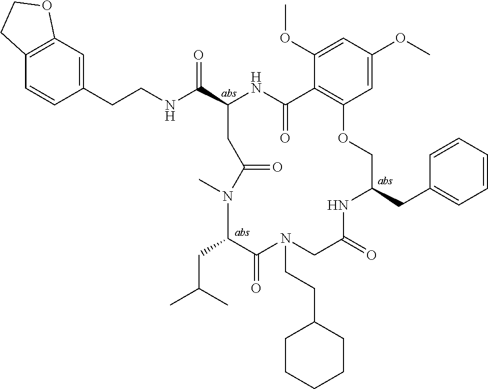 [SMILES: COc1cc(OC)c2c(c1)OC[C@@H](Cc1ccccc1)NC(=O)CN(CCC1CCCCC1)C(=O)[C@H](CC(C)C)N(C)C(=O)C[C@@H](C(=O)NCCc1ccc3c(c1)OCC3)NC2=O] | 276 |
| 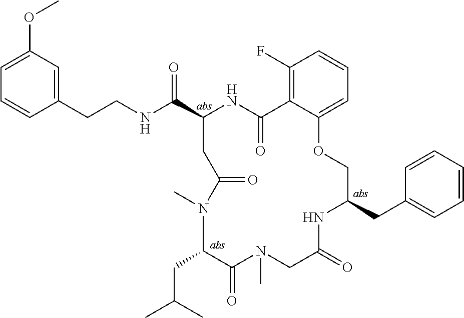 [SMILES: COc1cccc(CCNC(=O)[C@@H]2CC(=O)N(C)[C@@H](CC(C)C)C(=O)N(C)CC(=O)N[C@H](Cc3ccccc3)COc3cccc(F)c3C(=O)N2)c1] | 277 |
| 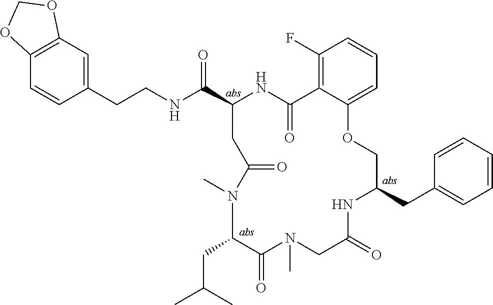 [SMILES: CC(C)C[C@H]1C(=O)N(C)CC(=O)N[C@H](Cc2ccccc2)COc2cccc(F)c2C(=O)N[C@H](C(=O)NCCc2ccc3c(c2)OCO3)CC(=O)N1C] | 278 |
| 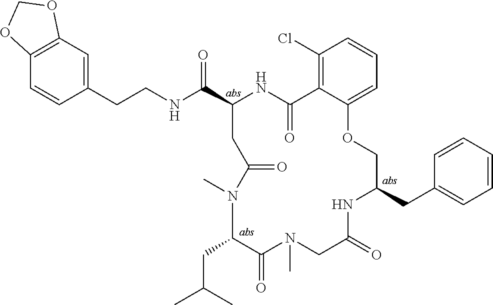 [SMILES: CC(C)C[C@H]1C(=O)N(C)CC(=O)N[C@H](Cc2ccccc2)COc2cccc(Cl)c2C(=O)N[C@H](C(=O)NCCc2ccc3c(c2)OCO3)CC(=O)N1C] | 279 |
| 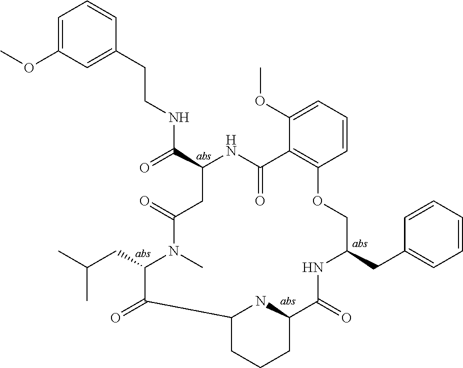 [SMILES: COc1cccc(CCNC(=O)[C@@H]2CC(=O)N(C)[C@@H](CC(C)C)C(=O)C3CCC[C@@H](N3)C(=O)N[C@H](Cc3ccccc3)COc3cccc(OC)c3C(=O)N2)c1] | 280 |
| 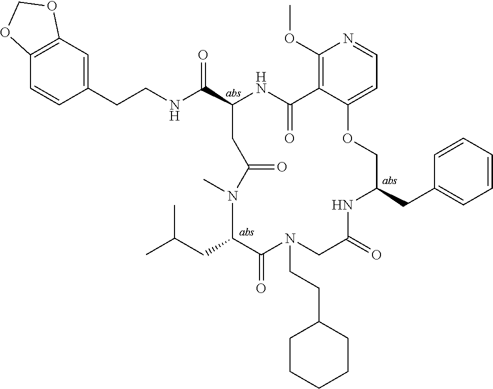 [SMILES: COc1nccc2c1C(=O)N[C@H](C(=O)NCCc1ccc3c(c1)OCO3)CC(=O)N(C)[C@@H](CC(C)C)C(=O)N(CCC1CCCCC1)CC(=O)N[C@H](Cc1ccccc1)CO2] | 281 |
| 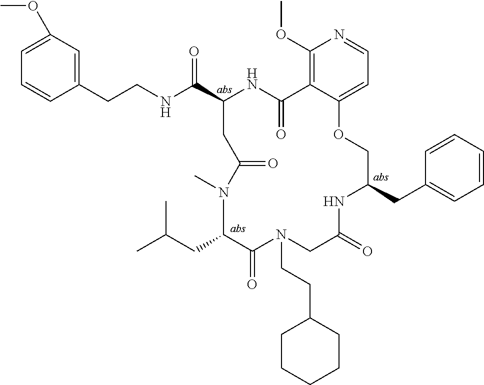 [SMILES: COc1cccc(CCNC(=O)[C@@H]2CC(=O)N(C)[C@@H](CC(C)C)C(=O)N(CCC3CCCCC3)CC(=O)N[C@H](Cc3ccccc3)COc3ccnc(OC)c3C(=O)N2)c1] | 282 |
| 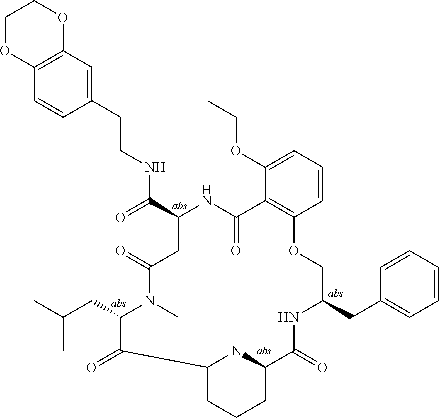 [SMILES: CCOc1cccc2c1C(=O)N[C@H](C(=O)NCCc1ccc3c(c1)OCCO3)CC(=O)N(C)[C@@H](CC(C)C)C(=O)C1CCC[C@@H](N1)C(=O)N[C@H](Cc1ccccc1)CO2] | 283 |
| 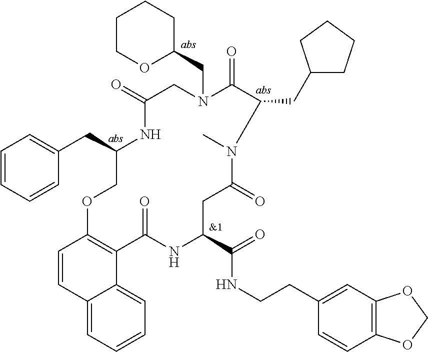 [SMILES: CN1C(=O)C[C@@H](C(=O)NCCc2ccc3c(c2)OCO3)NC(=O)c2c(ccc3ccccc23)OC[C@@H](Cc2ccccc2)NC(=O)CN(C[C@@H]2CCCCO2)C(=O)[C@@H]1CC1CCCC1] | 284 |
| 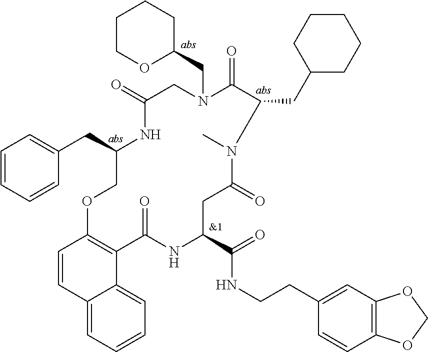 [SMILES: CN1C(=O)C[C@@H](C(=O)NCCc2ccc3c(c2)OCO3)NC(=O)c2c(ccc3ccccc23)OC[C@@H](Cc2ccccc2)NC(=O)CN(C[C@@H]2CCCCO2)C(=O)[C@@H]1CC1CCCCC1] | 285 |
| 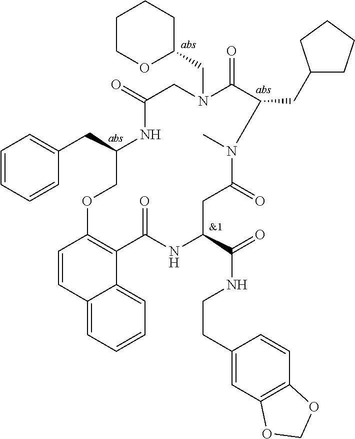 [SMILES: CN1C(=O)C[C@@H](C(=O)NCCc2ccc3c(c2)OCO3)NC(=O)c2c(ccc3ccccc23)OC[C@@H](Cc2ccccc2)NC(=O)CN(C[C@H]2CCCCO2)C(=O)[C@@H]1CC1CCCC1] | 286 |
| 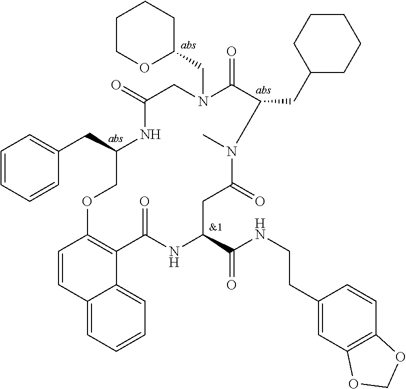 [SMILES: CN1C(=O)C[C@@H](C(=O)NCCc2ccc3c(c2)OCO3)NC(=O)c2c(ccc3ccccc23)OC[C@@H](Cc2ccccc2)NC(=O)CN(C[C@H]2CCCCO2)C(=O)[C@@H]1CC1CCCCC1] | 287 |
| 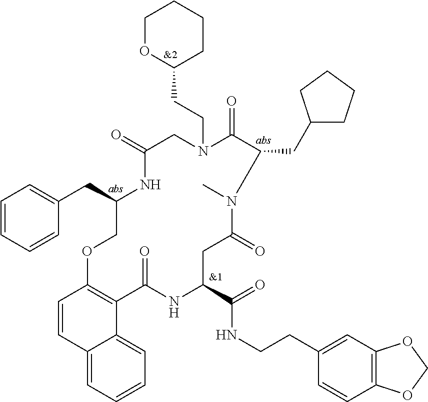 [SMILES: CN1C(=O)C[C@@H](C(=O)NCCc2ccc3c(c2)OCO3)NC(=O)c2c(ccc3ccccc23)OC[C@@H](Cc2ccccc2)NC(=O)CN(CC[C@H]2CCCCO2)C(=O)[C@@H]1CC1CCCC1] | 288 |
| 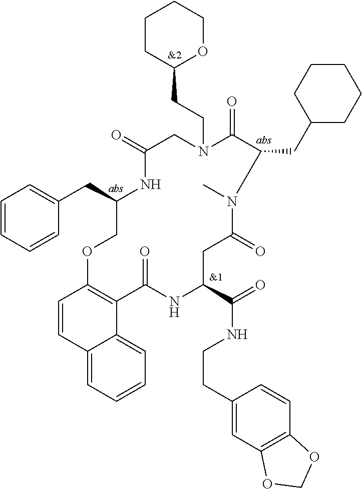 [SMILES: CN1C(=O)C[C@@H](C(=O)NCCc2ccc3c(c2)OCO3)NC(=O)c2c(ccc3ccccc23)OC[C@@H](Cc2ccccc2)NC(=O)CN(CC[C@H]2CCCCO2)C(=O)[C@@H]1CC1CCCCC1] | 289 |
| 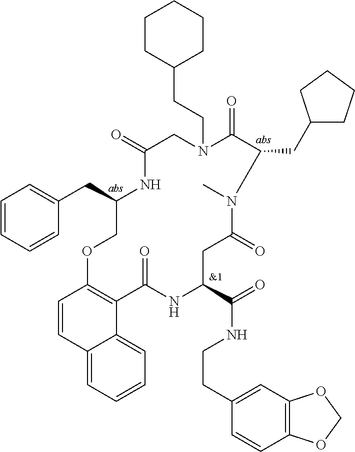 [SMILES: CN1C(=O)C[C@@H](C(=O)NCCc2ccc3c(c2)OCO3)NC(=O)c2c(ccc3ccccc23)OC[C@@H](Cc2ccccc2)NC(=O)CN(CCC2CCCCC2)C(=O)[C@@H]1CC1CCCC1] | 290 |
| 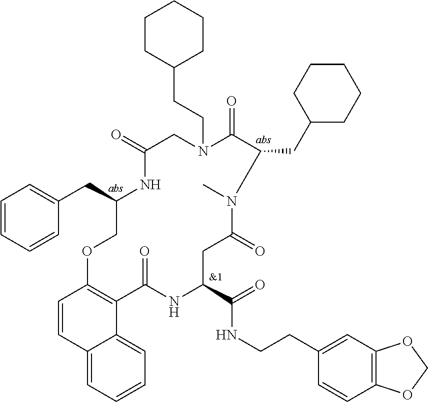 [SMILES: CN1C(=O)C[C@@H](C(=O)NCCc2ccc3c(c2)OCO3)NC(=O)c2c(ccc3ccccc23)OC[C@@H](Cc2ccccc2)NC(=O)CN(CCC2CCCCC2)C(=O)[C@@H]1CC1CCCCC1] | 291 |
| 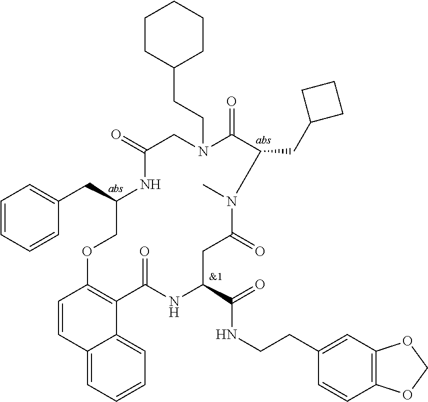 [SMILES: CN1C(=O)C[C@@H](C(=O)NCCc2ccc3c(c2)OCO3)NC(=O)c2c(ccc3ccccc23)OC[C@@H](Cc2ccccc2)NC(=O)CN(CCC2CCCCC2)C(=O)[C@@H]1CC1CCC1] | 292 |
| 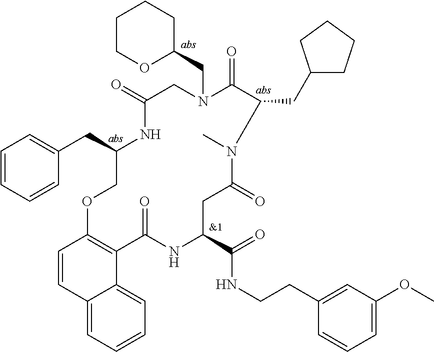 [SMILES: COc1cccc(CCNC(=O)[C@@H]2CC(=O)N(C)[C@@H](CC3CCCC3)C(=O)N(C[C@@H]3CCCCO3)CC(=O)N[C@H](Cc3ccccc3)COc3ccc4ccccc4c3C(=O)N2)c1] | 293 |
| 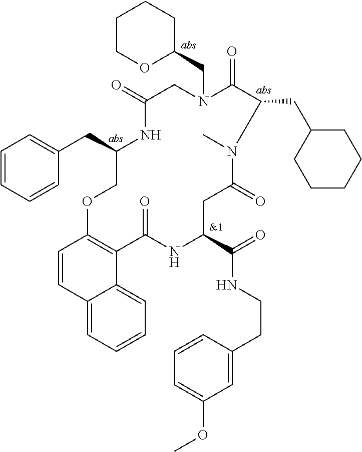 [SMILES: COc1cccc(CCNC(=O)[C@@H]2CC(=O)N(C)[C@@H](CC3CCCCC3)C(=O)N(C[C@@H]3CCCCO3)CC(=O)N[C@H](Cc3ccccc3)COc3ccc4ccccc4c3C(=O)N2)c1] | 294 |
| 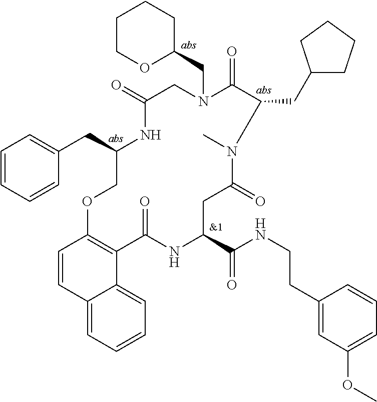 [SMILES: COc1cccc(CCNC(=O)[C@@H]2CC(=O)N(C)[C@@H](CC3CCCC3)C(=O)N(C[C@@H]3CCCCO3)CC(=O)N[C@H](Cc3ccccc3)COc3ccc4ccccc4c3C(=O)N2)c1] | 295 |
| 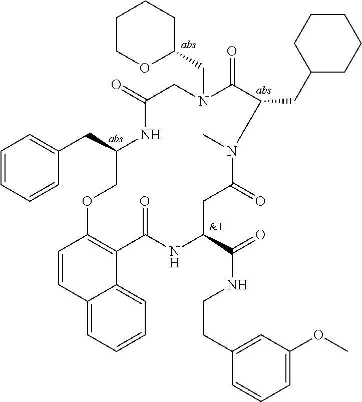 [SMILES: COc1cccc(CCNC(=O)[C@@H]2CC(=O)N(C)[C@@H](CC3CCCCC3)C(=O)N(C[C@H]3CCCCO3)CC(=O)N[C@H](Cc3ccccc3)COc3ccc4ccccc4c3C(=O)N2)c1] | 296 |
| 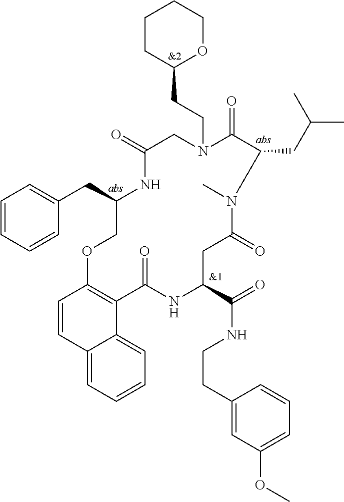 [SMILES: COc1cccc(CCNC(=O)[C@@H]2CC(=O)N(C)[C@@H](CC(C)C)C(=O)N(CC[C@H]3CCCCO3)CC(=O)N[C@H](Cc3ccccc3)COc3ccc4ccccc4c3C(=O)N2)c1] | 297 |
| 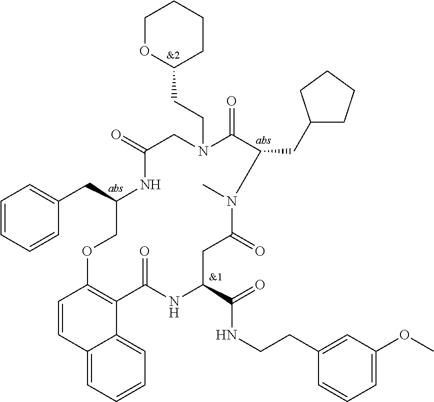 [SMILES: COc1cccc(CCNC(=O)[C@@H]2CC(=O)N(C)[C@@H](CC3CCCC3)C(=O)N(CC[C@H]3CCCCO3)CC(=O)N[C@H](Cc3ccccc3)COc3ccc4ccccc4c3C(=O)N2)c1] | 298 |
| 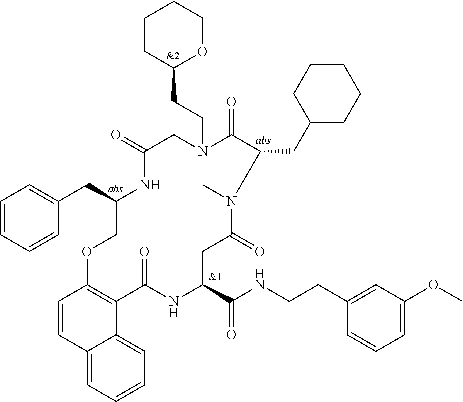 [SMILES: COc1cccc(CCNC(=O)[C@@H]2CC(=O)N(C)[C@@H](CC3CCCCC3)C(=O)N(CC[C@H]3CCCCO3)CC(=O)N[C@H](Cc3ccccc3)COc3ccc4ccccc4c3C(=O)N2)c1] | 299 |
| 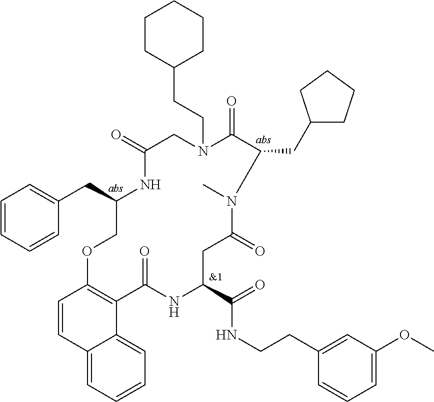 [SMILES: COc1cccc(CCNC(=O)[C@@H]2CC(=O)N(C)[C@@H](CC3CCCC3)C(=O)N(CCC3CCCCC3)CC(=O)N[C@H](Cc3ccccc3)COc3ccc4ccccc4c3C(=O)N2)c1] | 300 |
| 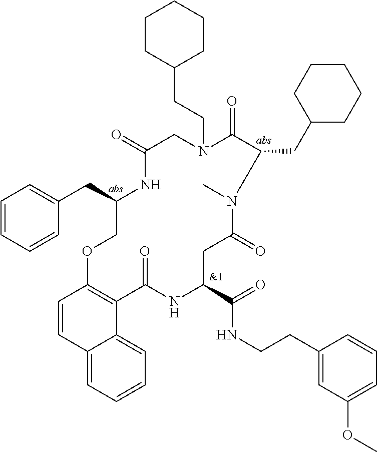 [SMILES: COc1cccc(CCNC(=O)[C@@H]2CC(=O)N(C)[C@@H](CC3CCCCC3)C(=O)N(CCC3CCCCC3)CC(=O)N[C@H](Cc3ccccc3)COc3ccc4ccccc4c3C(=O)N2)c1] | 301 |
| 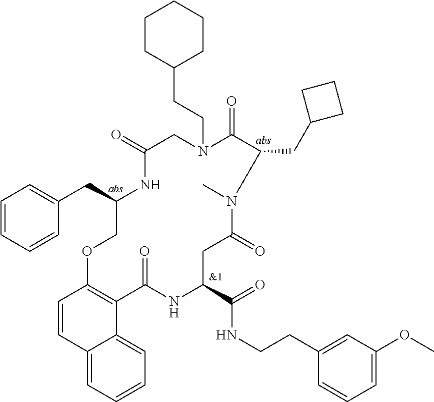 [SMILES: COc1cccc(CCNC(=O)[C@@H]2CC(=O)N(C)[C@@H](CC3CCC3)C(=O)N(CCC3CCCCC3)CC(=O)N[C@H](Cc3ccccc3)COc3ccc4ccccc4c3C(=O)N2)c1] | 302 |
| 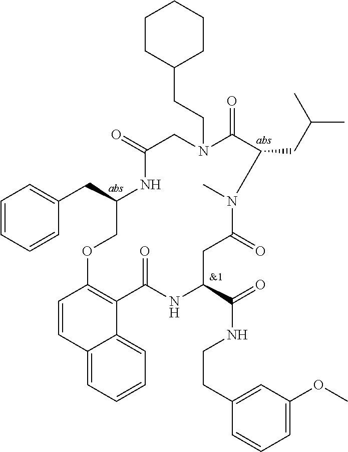 [SMILES: COc1cccc(CCNC(=O)[C@@H]2CC(=O)N(C)[C@@H](CC(C)C)C(=O)N(CCC3CCCCC3)CC(=O)N[C@H](Cc3ccccc3)COc3ccc4ccccc4c3C(=O)N2)c1] | 303 |
| 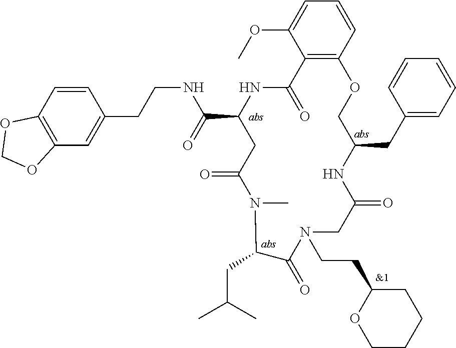 [SMILES: COc1cccc2c1C(=O)N[C@H](C(=O)NCCc1ccc3c(c1)OCO3)CC(=O)N(C)[C@@H](CC(C)C)C(=O)N(CC[C@H]1CCCCO1)CC(=O)N[C@H](Cc1ccccc1)CO2] | 304 |
| 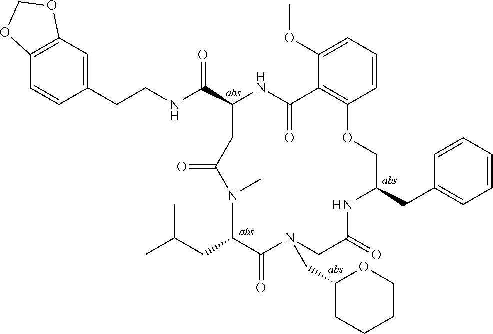 [SMILES: COc1cccc2c1C(=O)N[C@H](C(=O)NCCc1ccc3c(c1)OCO3)CC(=O)N(C)[C@@H](CC(C)C)C(=O)N(C[C@H]1CCCCO1)CC(=O)N[C@H](Cc1ccccc1)CO2] | 305 |
| 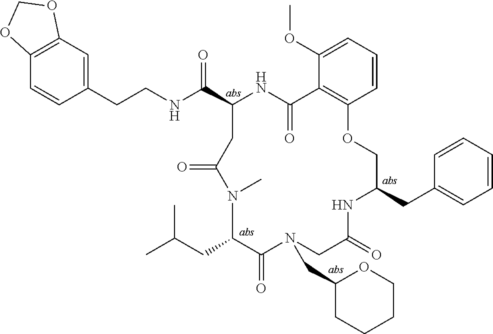 [SMILES: COc1cccc2c1C(=O)N[C@H](C(=O)NCCc1ccc3c(c1)OCO3)CC(=O)N(C)[C@@H](CC(C)C)C(=O)N(C[C@@H]1CCCCO1)CC(=O)N[C@H](Cc1ccccc1)CO2] | 306 |
| 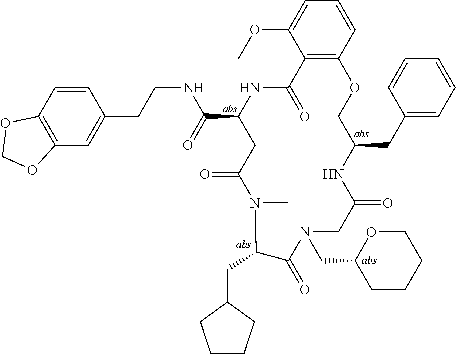 [SMILES: COc1cccc2c1C(=O)N[C@H](C(=O)NCCc1ccc3c(c1)OCO3)CC(=O)N(C)[C@@H](CC1CCCC1)C(=O)N(C[C@H]1CCCCO1)CC(=O)N[C@H](Cc1ccccc1)CO2] | 307 |
| 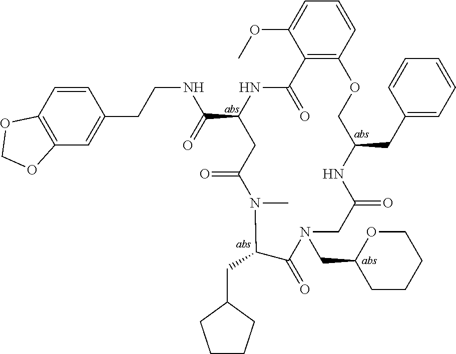 [SMILES: COc1cccc2c1C(=O)N[C@H](C(=O)NCCc1ccc3c(c1)OCO3)CC(=O)N(C)[C@@H](CC1CCCC1)C(=O)N(C[C@@H]1CCCCO1)CC(=O)N[C@H](Cc1ccccc1)CO2] | 308 |
| 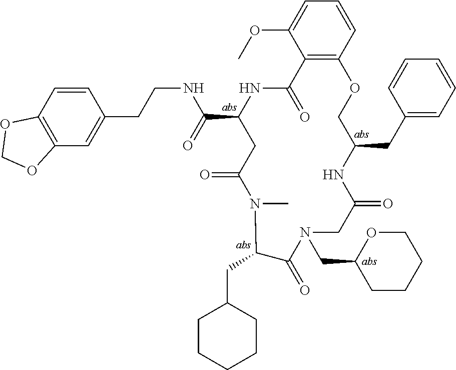 [SMILES: COc1cccc2c1C(=O)N[C@H](C(=O)NCCc1ccc3c(c1)OCO3)CC(=O)N(C)[C@@H](CC1CCCCC1)C(=O)N(C[C@@H]1CCCCO1)CC(=O)N[C@H](Cc1ccccc1)CO2] | 309 |
| 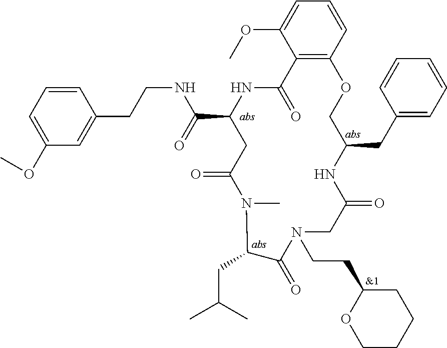 [SMILES: COc1cccc(CCNC(=O)[C@@H]2CC(=O)N(C)[C@@H](CC(C)C)C(=O)N(CC[C@H]3CCCCO3)CC(=O)N[C@H](Cc3ccccc3)COc3cccc(OC)c3C(=O)N2)c1] | 310 |
| 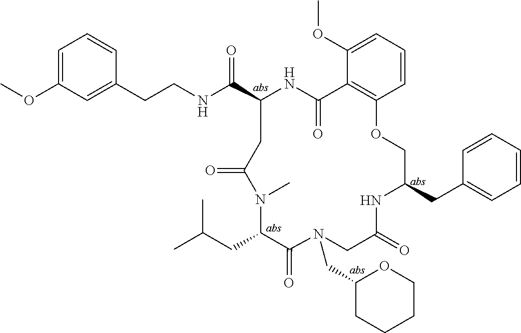 [SMILES: COc1cccc(CCNC(=O)[C@@H]2CC(=O)N(C)[C@@H](CC(C)C)C(=O)N(C[C@H]3CCCCO3)CC(=O)N[C@H](Cc3ccccc3)COc3cccc(OC)c3C(=O)N2)c1] | 311 |
| 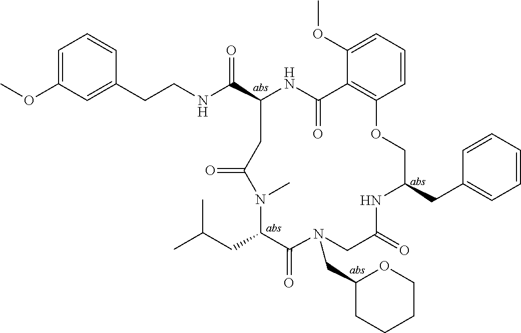 [SMILES: COc1cccc(CCNC(=O)[C@@H]2CC(=O)N(C)[C@@H](CC(C)C)C(=O)N(C[C@@H]3CCCCO3)CC(=O)N[C@H](Cc3ccccc3)COc3cccc(OC)c3C(=O)N2)c1] | 312 |
| 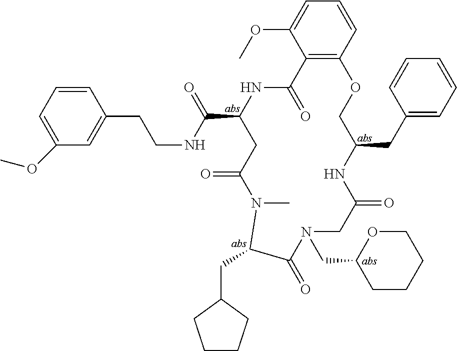 [SMILES: COc1cccc(CCNC(=O)[C@@H]2CC(=O)N(C)[C@@H](CC3CCCC3)C(=O)N(C[C@H]3CCCCO3)CC(=O)N[C@H](Cc3ccccc3)COc3cccc(OC)c3C(=O)N2)c1] | 313 |
| 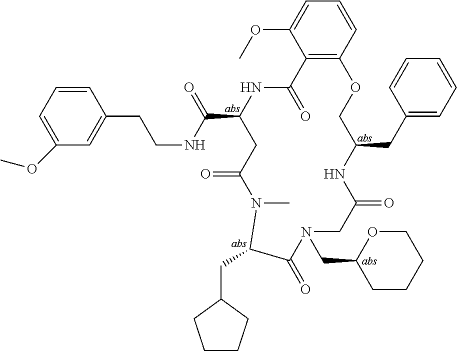 [SMILES: COc1cccc(CCNC(=O)[C@@H]2CC(=O)N(C)[C@@H](CC3CCCC3)C(=O)N(C[C@@H]3CCCCO3)CC(=O)N[C@H](Cc3ccccc3)COc3cccc(OC)c3C(=O)N2)c1] | 314 |
| 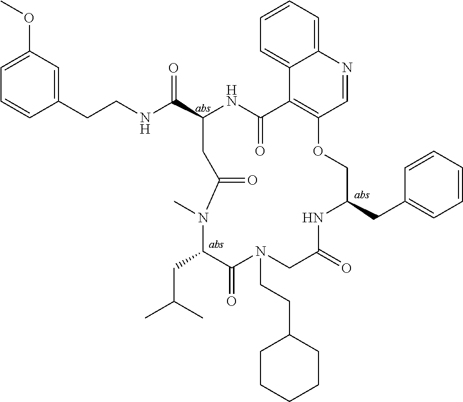 [SMILES: COc1cccc(CCNC(=O)[C@@H]2CC(=O)N(C)[C@@H](CC(C)C)C(=O)N(CCC3CCCCC3)CC(=O)N[C@H](Cc3ccccc3)COc3cnc4ccccc4c3C(=O)N2)c1] | 315 |
| 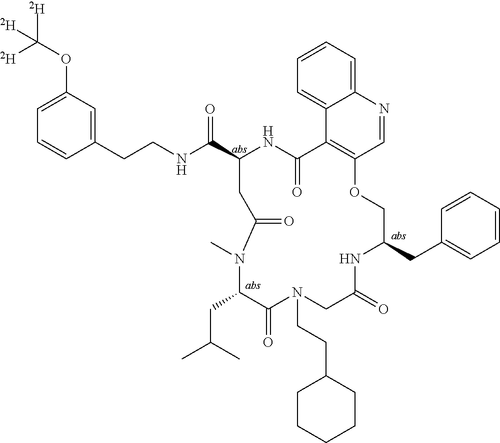 [SMILES: [2H]C([2H])([2H])Oc1cccc(CCNC(=O)[C@@H]2CC(=O)N(C)[C@@H](CC(C)C)C(=O)N(CCC3CCCCC3)CC(=O)N[C@H](Cc3ccccc3)COc3cnc4ccccc4c3C(=O)N2)c1] | 316 |
| 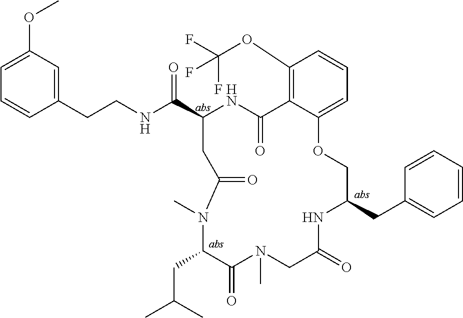 [SMILES: COc1cccc(CCNC(=O)[C@@H]2CC(=O)N(C)[C@@H](CC(C)C)C(=O)N(C)CC(=O)N[C@H](Cc3ccccc3)COc3cccc(OC(F)(F)F)c3C(=O)N2)c1] | 317 |
| 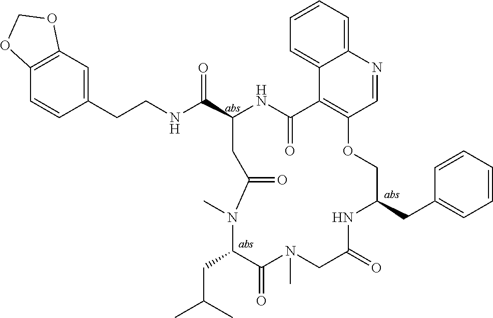 [SMILES: CC(C)C[C@H]1C(=O)N(C)CC(=O)N[C@H](Cc2ccccc2)COc2cnc3ccccc3c2C(=O)N[C@H](C(=O)NCCc2ccc3c(c2)OCO3)CC(=O)N1C] | 318 |
| 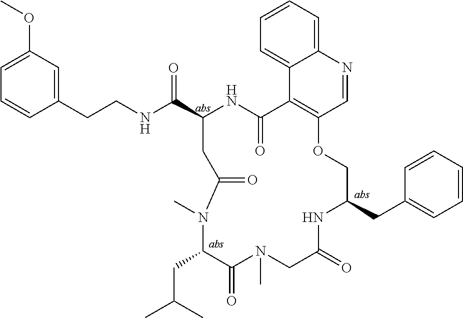 [SMILES: COc1cccc(CCNC(=O)[C@@H]2CC(=O)N(C)[C@@H](CC(C)C)C(=O)N(C)CC(=O)N[C@H](Cc3ccccc3)COc3cnc4ccccc4c3C(=O)N2)c1] | 319 |
| 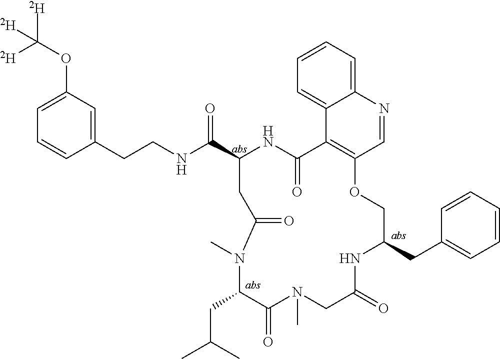 [SMILES: [2H]C([2H])([2H])Oc1cccc(CCNC(=O)[C@@H]2CC(=O)N(C)[C@@H](CC(C)C)C(=O)N(C)CC(=O)N[C@H](Cc3ccccc3)COc3cnc4ccccc4c3C(=O)N2)c1] | 320 |
| 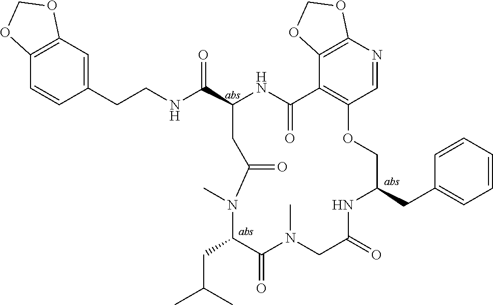 [SMILES: CC(C)C[C@H]1C(=O)N(C)CC(=O)N[C@H](Cc2ccccc2)COc2cnc3c(c2C(=O)N[C@H](C(=O)NCCc2ccc4c(c2)OCO4)CC(=O)N1C)OCO3] | 321 |
| 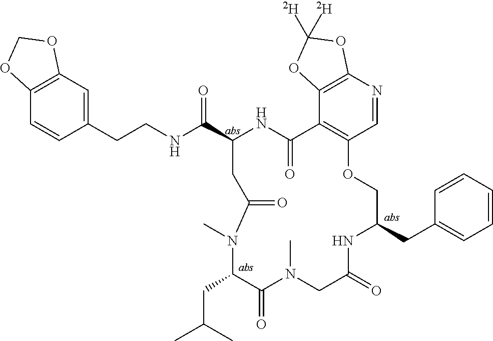 [SMILES: [2H]C1([2H])Oc2ncc3c(c2O1)C(=O)N[C@H](C(=O)NCCc1ccc2c(c1)OCO2)CC(=O)N(C)[C@@H](CC(C)C)C(=O)N(C)CC(=O)N[C@H](Cc1ccccc1)CO3] | 322 |
| 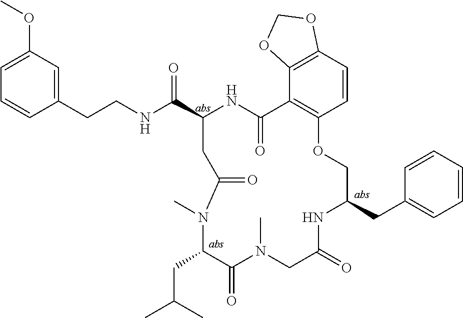 [SMILES: COc1cccc(CCNC(=O)[C@@H]2CC(=O)N(C)[C@@H](CC(C)C)C(=O)N(C)CC(=O)N[C@H](Cc3ccccc3)COc3ccc4c(c3C(=O)N2)OCO4)c1] | 323 |
| 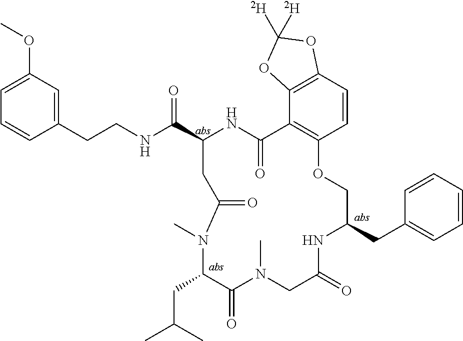 [SMILES: [2H]C1([2H])Oc2ccc3c(c2O1)C(=O)N[C@H](C(=O)NCCc1cccc(OC)c1)CC(=O)N(C)[C@@H](CC(C)C)C(=O)N(C)CC(=O)N[C@H](Cc1ccccc1)CO3] | 324 |
| 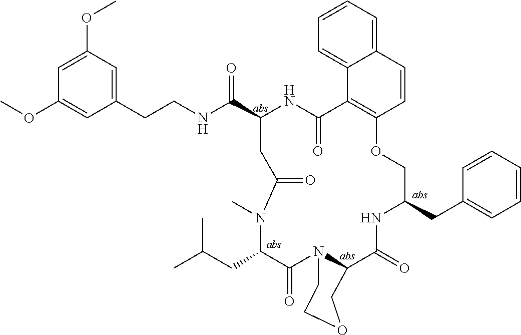 | 325 |
| 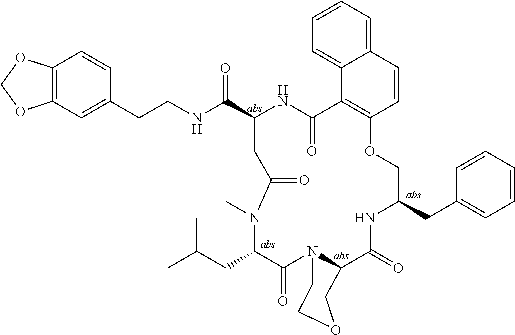 | 326 |
| 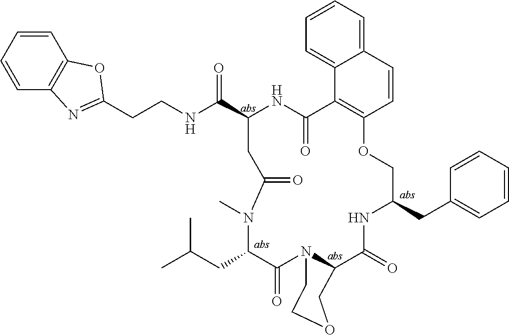 | 327 |
| 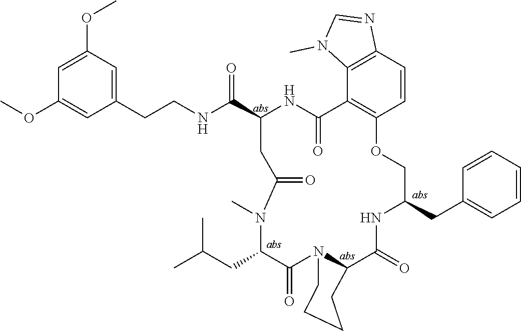 | 328 |
| 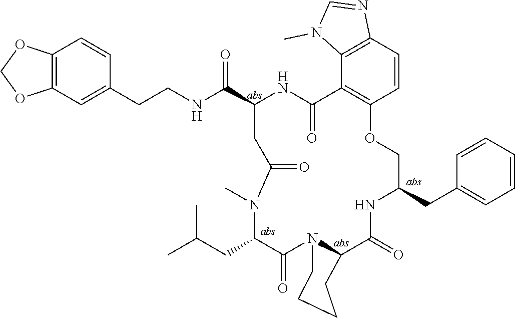 | 329 |
| 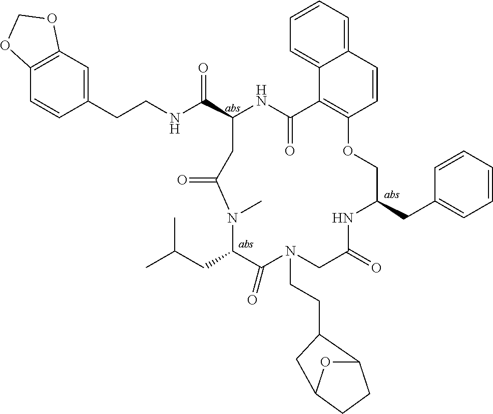 [SMILES: CC(C)C[C@H]1C(=O)N(CCC2CC3CCC2O3)CC(=O)N[C@H](Cc2ccccc2)COc2ccc3ccccc3c2C(=O)N[C@H](C(=O)NCCc2ccc3c(c2)OCO3)CC(=O)N1C] | 330 |
| 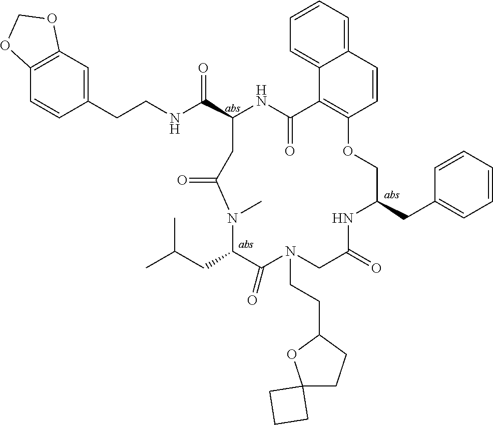 [SMILES: CC(C)C[C@H]1C(=O)N(CCC2CCC3(CCC3)O2)CC(=O)N[C@H](Cc2ccccc2)COc2ccc3ccccc3c2C(=O)N[C@H](C(=O)NCCc2ccc3c(c2)OCO3)CC(=O)N1C] | 331 |
| 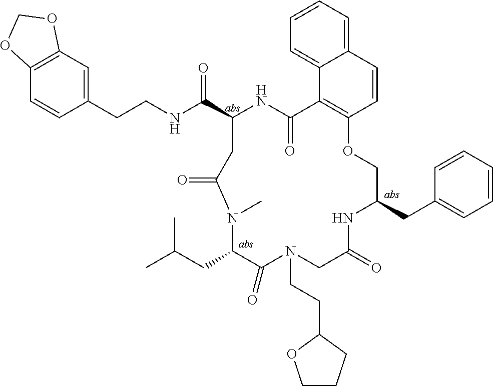 [SMILES: CC(C)C[C@H]1C(=O)N(CCC2CCCO2)CC(=O)N[C@H](Cc2ccccc2)COc2ccc3ccccc3c2C(=O)N[C@H](C(=O)NCCc2ccc3c(c2)OCO3)CC(=O)N1C] | 332 |
| 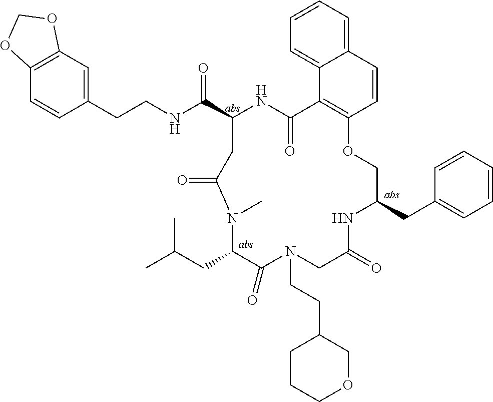 [SMILES: CC(C)C[C@H]1C(=O)N(CCC2CCCOC2)CC(=O)N[C@H](Cc2ccccc2)COc2ccc3ccccc3c2C(=O)N[C@H](C(=O)NCCc2ccc3c(c2)OCO3)CC(=O)N1C] | 333 |
| 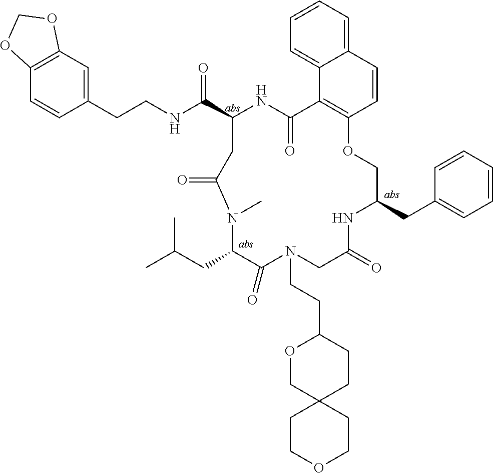 [SMILES: CC(C)C[C@H]1C(=O)N(CCC2CCC3(CCOCC3)CO2)CC(=O)N[C@H](Cc2ccccc2)COc2ccc3ccccc3c2C(=O)N[C@H](C(=O)NCCc2ccc3c(c2)OCO3)CC(=O)N1C] | 334 |
| 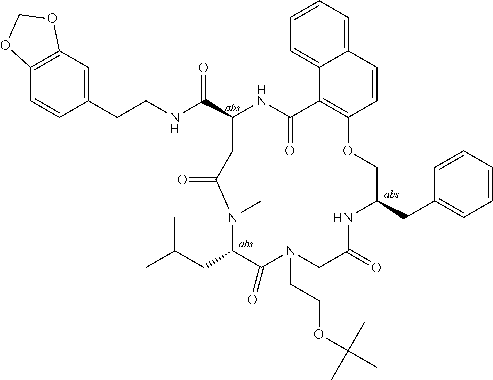 [SMILES: CC(C)C[C@H]1C(=O)N(CCOC(C)(C)C)CC(=O)N[C@H](Cc2ccccc2)COc2ccc3ccccc3c2C(=O)N[C@H](C(=O)NCCc2ccc3c(c2)OCO3)CC(=O)N1C] | 335 |
| 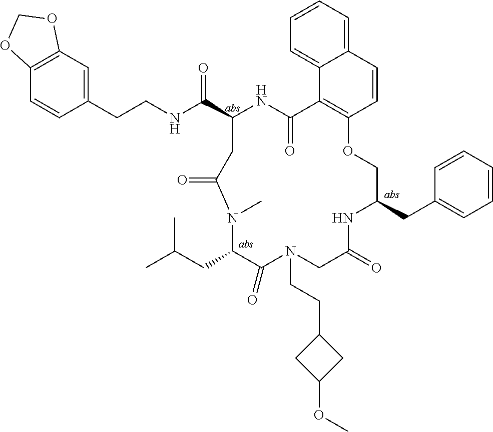 [SMILES: COC1CC(CCN2CC(=O)N[C@H](Cc3ccccc3)COc3ccc4ccccc4c3C(=O)N[C@H](C(=O)NCCc3ccc4c(c3)OCO4)CC(=O)N(C)[C@@H](CC(C)C)C2=O)C1] | 336 |
| 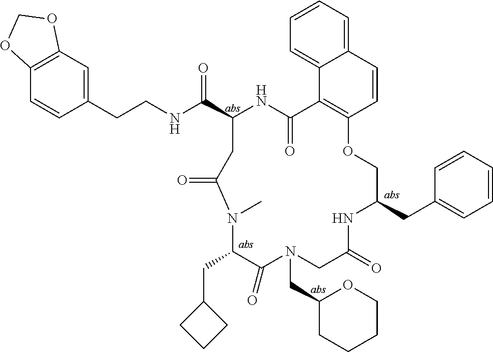 [SMILES: CN1C(=O)C[C@@H](C(=O)NCCc2ccc3c(c2)OCO3)NC(=O)c2c(ccc3ccccc23)OC[C@@H](Cc2ccccc2)NC(=O)CN(C[C@@H]2CCCCO2)C(=O)[C@@H]1CC1CCC1] | 337 |
| 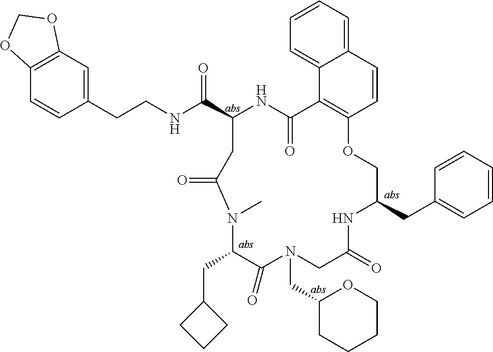 [SMILES: CN1C(=O)C[C@@H](C(=O)NCCc2ccc3c(c2)OCO3)NC(=O)c2c(ccc3ccccc23)OC[C@@H](Cc2ccccc2)NC(=O)CN(C[C@H]2CCCCO2)C(=O)[C@@H]1CC1CCC1] | 338 |
| 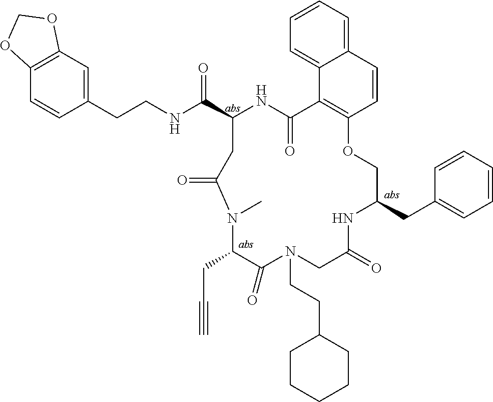 [SMILES: C#CC[C@H]1C(=O)N(CCC2CCCCC2)CC(=O)N[C@H](Cc2ccccc2)COc2ccc3ccccc3c2C(=O)N[C@H](C(=O)NCCc2ccc3c(c2)OCO3)CC(=O)N1C] | 339 |
| 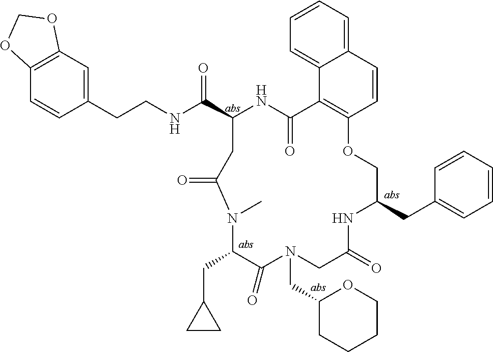 [SMILES: CN1C(=O)C[C@@H](C(=O)NCCc2ccc3c(c2)OCO3)NC(=O)c2c(ccc3ccccc23)OC[C@@H](Cc2ccccc2)NC(=O)CN(C[C@H]2CCCCO2)C(=O)[C@@H]1CC1CC1] | 340 |
| 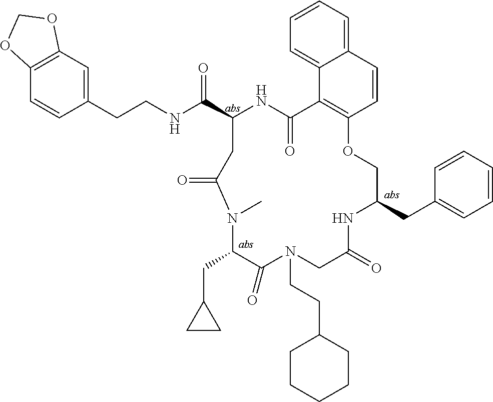 [SMILES: CN1C(=O)C[C@@H](C(=O)NCCc2ccc3c(c2)OCO3)NC(=O)c2c(ccc3ccccc23)OC[C@@H](Cc2ccccc2)NC(=O)CN(CCC2CCCCC2)C(=O)[C@@H]1CC1CC1] | 341 |
| 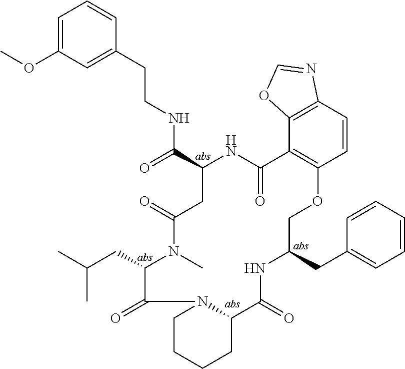 [SMILES: COc1cccc(CCNC(=O)[C@@H]2CC(=O)N(C)[C@@H](CC(C)C)C(=O)N3CCCC[C@H]3C(=O)N[C@H](Cc3ccccc3)COc3ccc4ncoc4c3C(=O)N2)c1] | 342 |
| 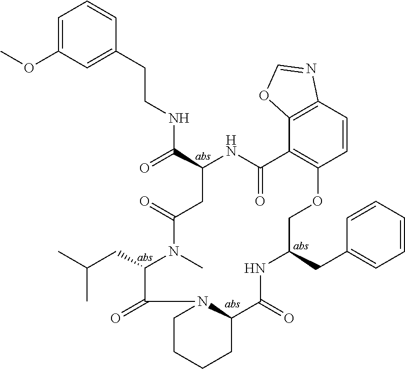 [SMILES: COc1cccc(CCNC(=O)[C@@H]2CC(=O)N(C)[C@@H](CC(C)C)C(=O)N3CCCC[C@@H]3C(=O)N[C@H](Cc3ccccc3)COc3ccc4ncoc4c3C(=O)N2)c1] | 343 |
| 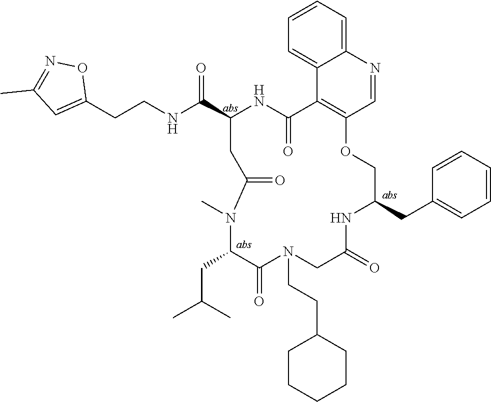 [SMILES: Cc1cc(CCNC(=O)[C@@H]2CC(=O)N(C)[C@@H](CC(C)C)C(=O)N(CCC3CCCCC3)CC(=O)N[C@H](Cc3ccccc3)COc3cnc4ccccc4c3C(=O)N2)on1] | 344 |
| 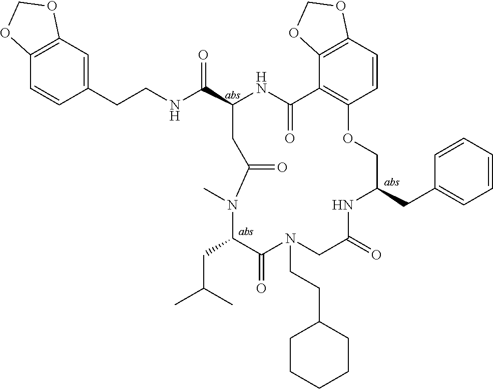 [SMILES: CC(C)C[C@H]1C(=O)N(CCC2CCCCC2)CC(=O)N[C@H](Cc2ccccc2)COc2ccc3c(c2C(=O)N[C@H](C(=O)NCCc2ccc4c(c2)OCO4)CC(=O)N1C)OCO3] | 345 |
| 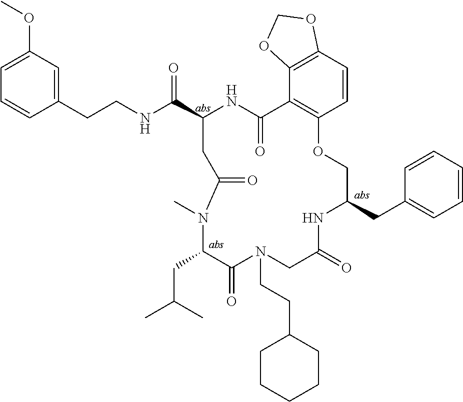 [SMILES: COc1cccc(CCNC(=O)[C@@H]2CC(=O)N(C)[C@@H](CC(C)C)C(=O)N(CCC3CCCCC3)CC(=O)N[C@H](Cc3ccccc3)COc3ccc4c(c3C(=O)N2)OCO4)c1] | 346 |
| 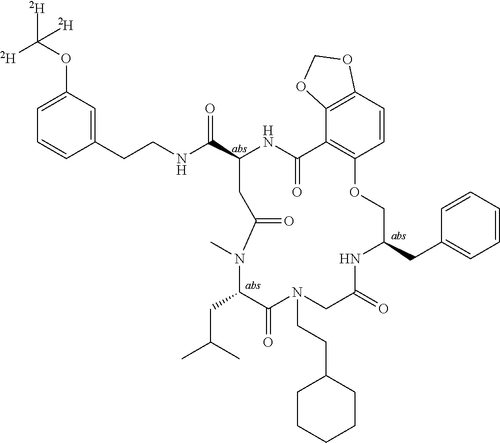 [SMILES: [2H]C([2H])([2H])Oc1cccc(CCNC(=O)[C@@H]2CC(=O)N(C)[C@@H](CC(C)C)C(=O)N(CCC3CCCCC3)CC(=O)N[C@H](Cc3ccccc3)COc3ccc4c(c3C(=O)N2)OCO4)c1] | 347 |
| 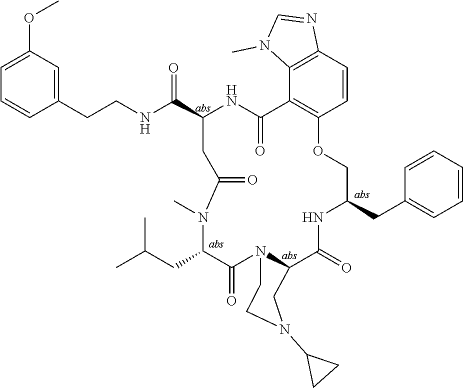 | 348 |
| 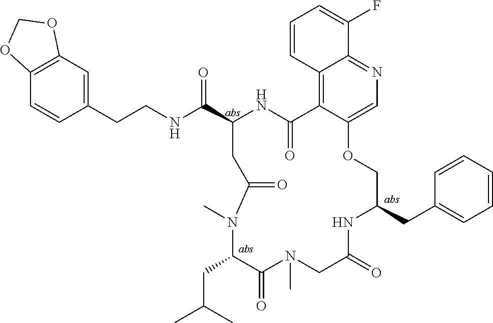 [SMILES: CC(C)C[C@H]1C(=O)N(C)CC(=O)N[C@H](Cc2ccccc2)COc2cnc3c(F)cccc3c2C(=O)N[C@H](C(=O)NCCc2ccc3c(c2)OCO3)CC(=O)N1C] | 349 |
| 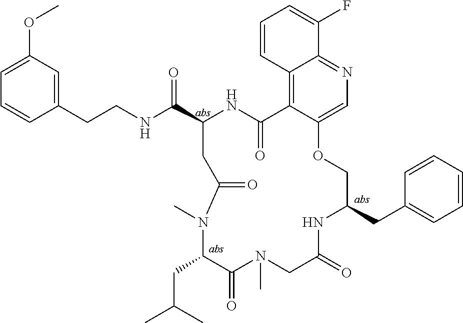 [SMILES: COc1cccc(CCNC(=O)[C@@H]2CC(=O)N(C)[C@@H](CC(C)C)C(=O)N(C)CC(=O)N[C@H](Cc3ccccc3)COc3cnc4c(F)cccc4c3C(=O)N2)c1] | 350 |
| 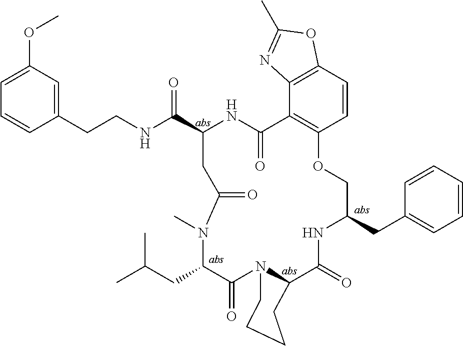 | 351 |
| 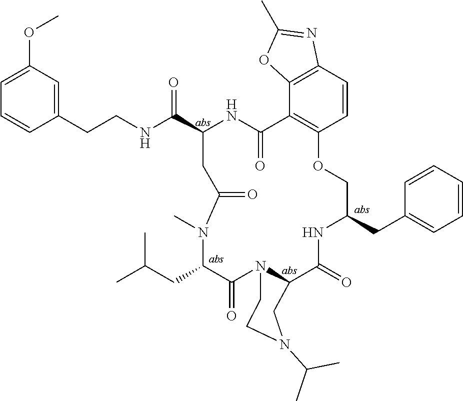 | 352 |
| 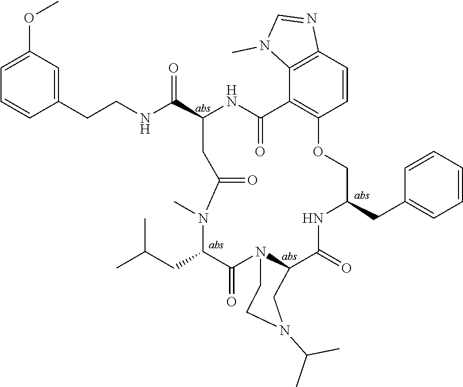 | 353 |
| 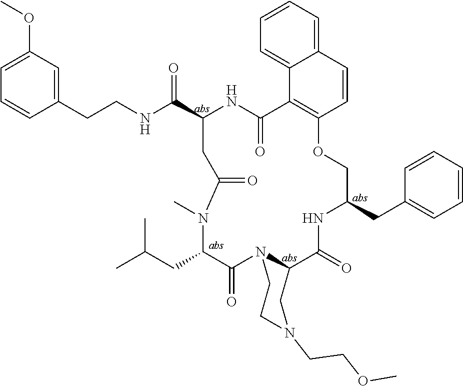 | 354 |
| 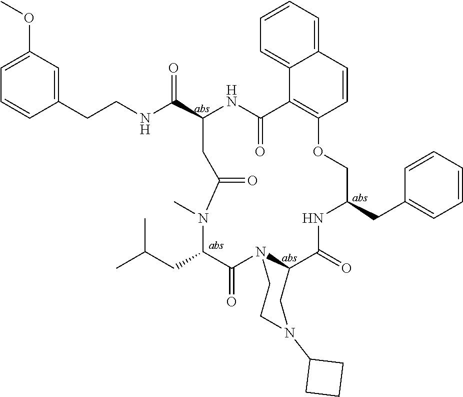 | 355 |
| 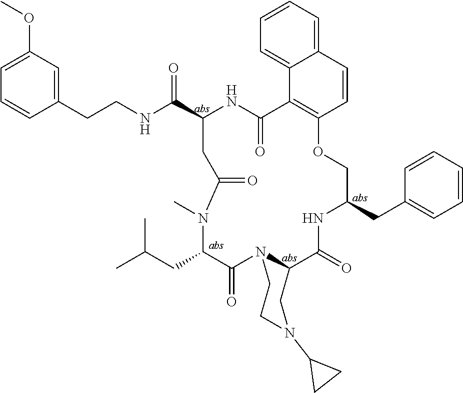 | 356 |
| 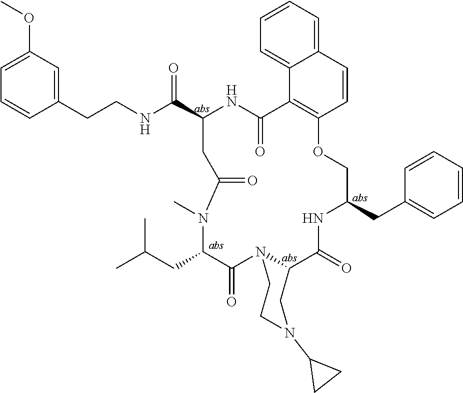 | 357 |
| 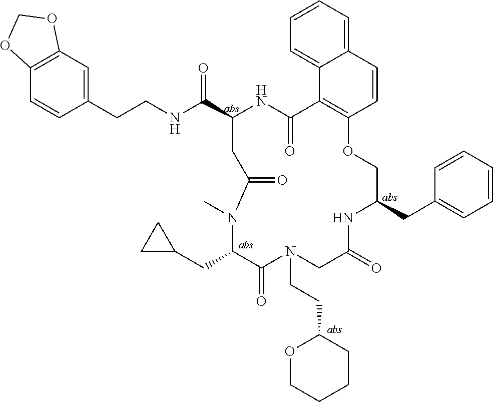 [SMILES: CN1C(=O)C[C@@H](C(=O)NCCc2ccc3c(c2)OCO3)NC(=O)c2c(ccc3ccccc23)OC[C@@H](Cc2ccccc2)NC(=O)CN(CC[C@@H]2CCCCO2)C(=O)[C@@H]1CC1CC1] | 358 |
| 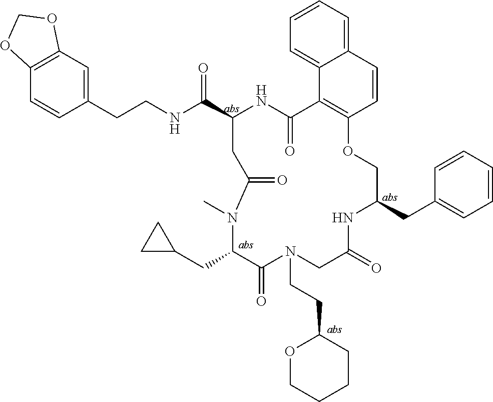 [SMILES: CN1C(=O)C[C@@H](C(=O)NCCc2ccc3c(c2)OCO3)NC(=O)c2c(ccc3ccccc23)OC[C@@H](Cc2ccccc2)NC(=O)CN(CC[C@H]2CCCCO2)C(=O)[C@@H]1CC1CC1] | 359 |
| 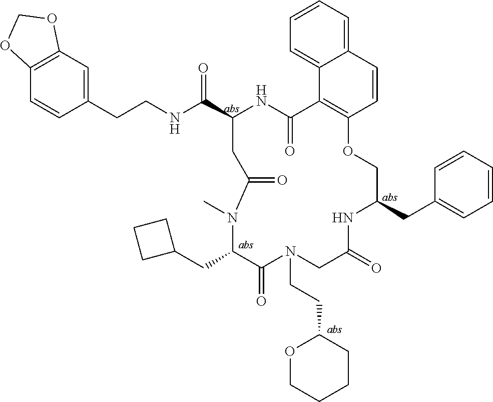 [SMILES: CN1C(=O)C[C@@H](C(=O)NCCc2ccc3c(c2)OCO3)NC(=O)c2c(ccc3ccccc23)OC[C@@H](Cc2ccccc2)NC(=O)CN(CC[C@@H]2CCCCO2)C(=O)[C@@H]1CC1CCC1] | 360 |
| 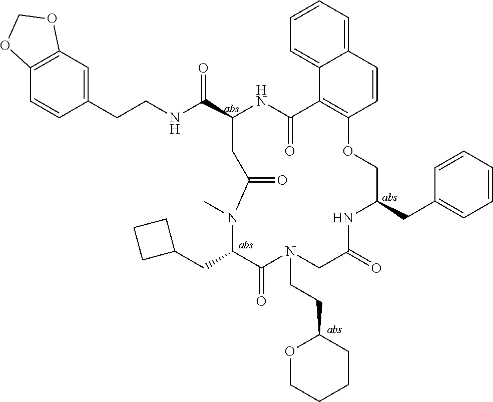 [SMILES: CN1C(=O)C[C@@H](C(=O)NCCc2ccc3c(c2)OCO3)NC(=O)c2c(ccc3ccccc23)OC[C@@H](Cc2ccccc2)NC(=O)CN(CC[C@H]2CCCCO2)C(=O)[C@@H]1CC1CCC1] | 361 |
| 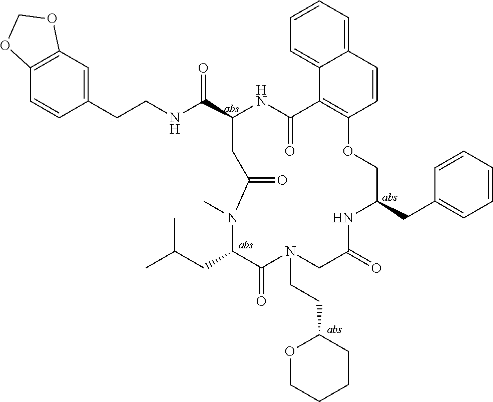 [SMILES: CC(C)C[C@H]1C(=O)N(CC[C@@H]2CCCCO2)CC(=O)N[C@H](Cc2ccccc2)COc2ccc3ccccc3c2C(=O)N[C@H](C(=O)NCCc2ccc3c(c2)OCO3)CC(=O)N1C] | 362 |
| 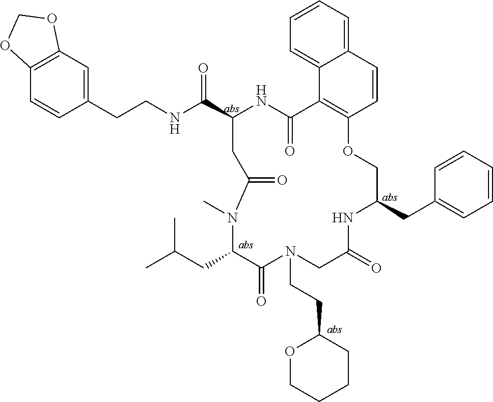 [SMILES: CC(C)C[C@H]1C(=O)N(CC[C@H]2CCCCO2)CC(=O)N[C@H](Cc2ccccc2)COc2ccc3ccccc3c2C(=O)N[C@H](C(=O)NCCc2ccc3c(c2)OCO3)CC(=O)N1C] | 363 |
| 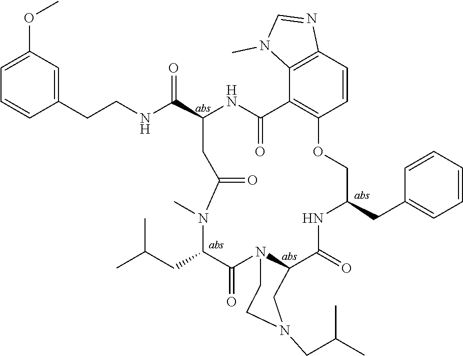 | 364 |
| 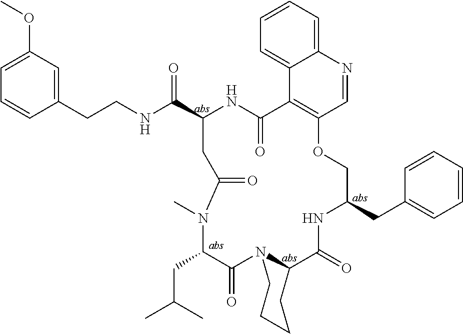 | 365 |
| 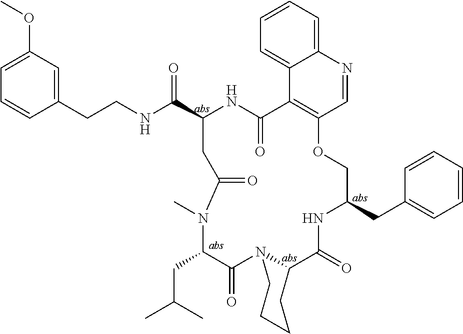 | 366 |
| 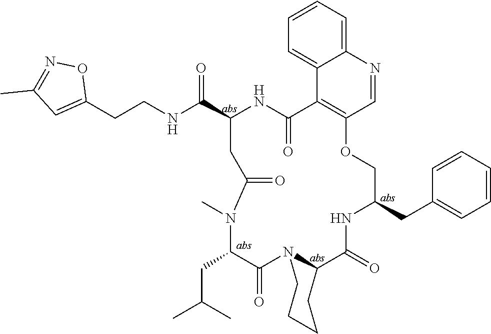 | 367 |
| 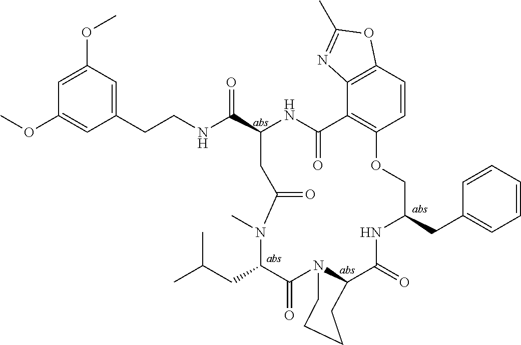 | 368 |
| 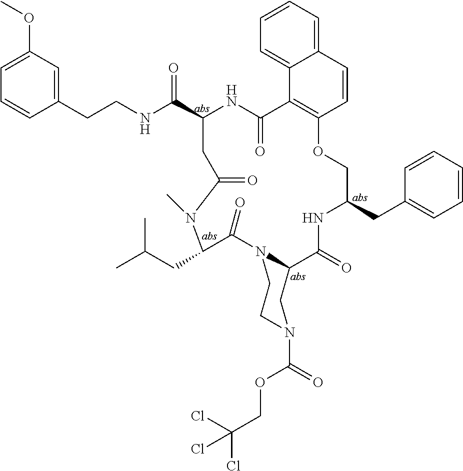 | 369 |
| 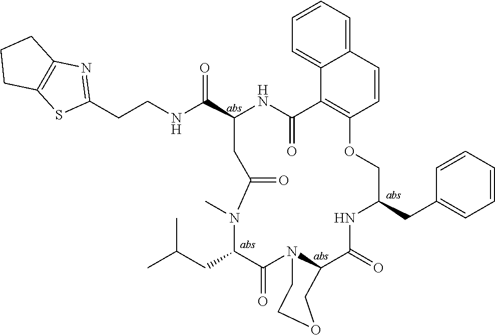 | 370 |
| 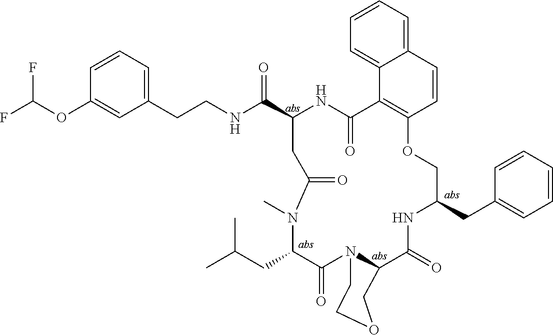 | 371 |
| 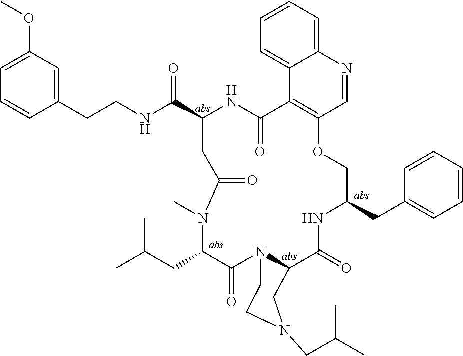 | 372 |
| 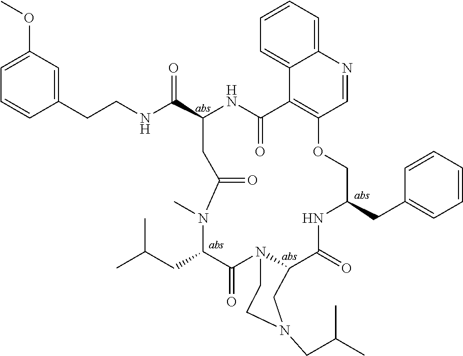 | 373 |
| 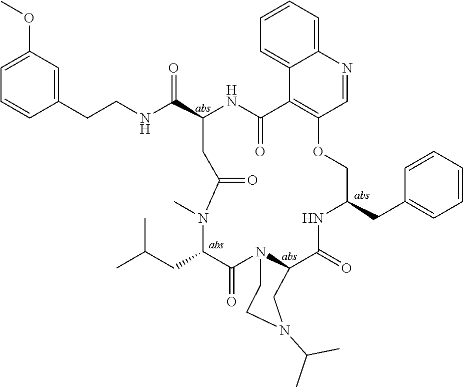 | 374 |
| 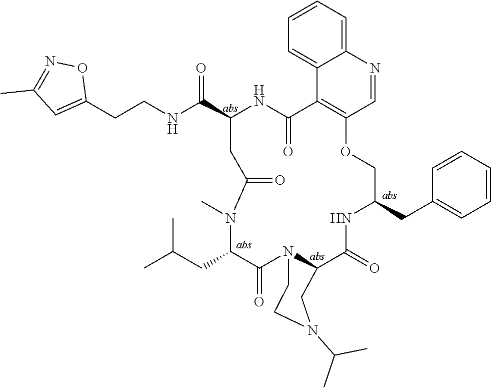 | 375 |
| 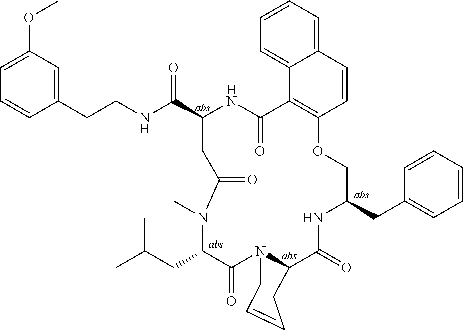 | 376 |
| 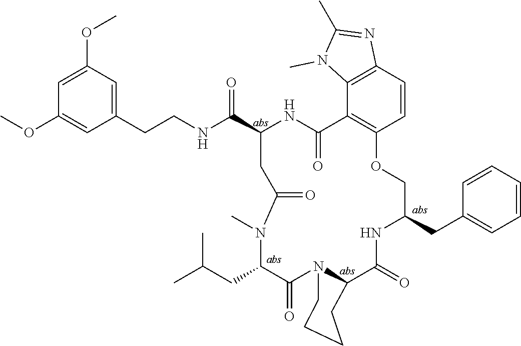 | 377 |
| 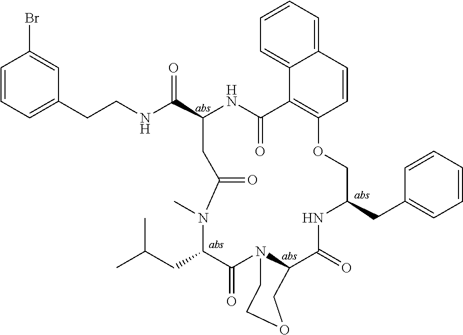 | 378 |
| 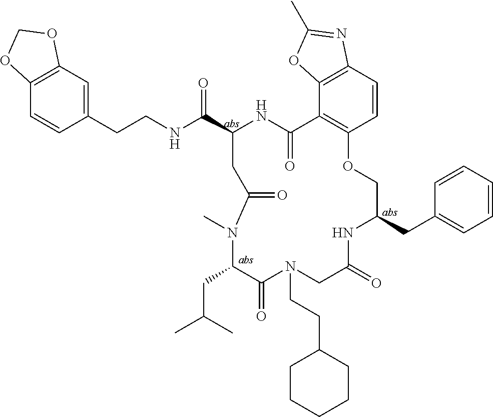 [SMILES: Cc1nc2ccc3c(c2o1)C(=O)N[C@H](C(=O)NCCc1ccc2c(c1)OCO2)CC(=O)N(C)[C@@H](CC(C)C)C(=O)N(CCC1CCCCC1)CC(=O)N[C@H](Cc1ccccc1)CO3] | 379 |
| 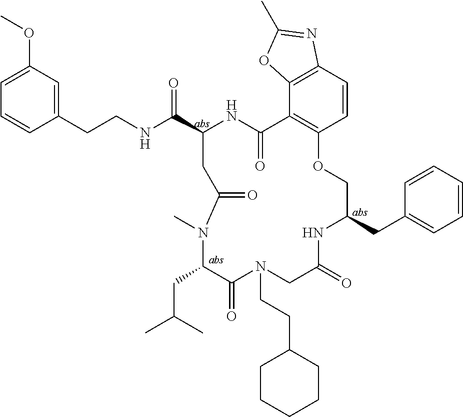 [SMILES: COc1cccc(CCNC(=O)[C@@H]2CC(=O)N(C)[C@@H](CC(C)C)C(=O)N(CCC3CCCCC3)CC(=O)N[C@H](Cc3ccccc3)COc3ccc4nc(C)oc4c3C(=O)N2)c1] | 380 |
| 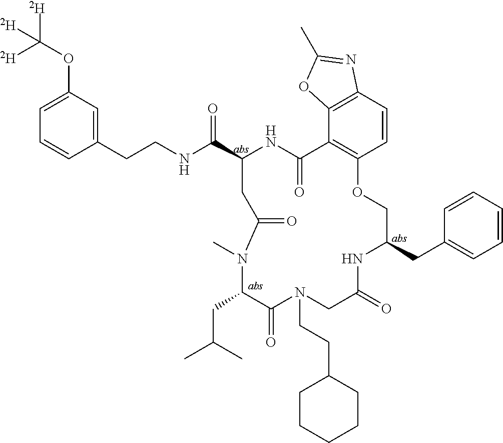 [SMILES: [2H]C([2H])([2H])Oc1cccc(CCNC(=O)[C@@H]2CC(=O)N(C)[C@@H](CC(C)C)C(=O)N(CCC3CCCCC3)CC(=O)N[C@H](Cc3ccccc3)COc3ccc4nc(C)oc4c3C(=O)N2)c1] | 381 |
| 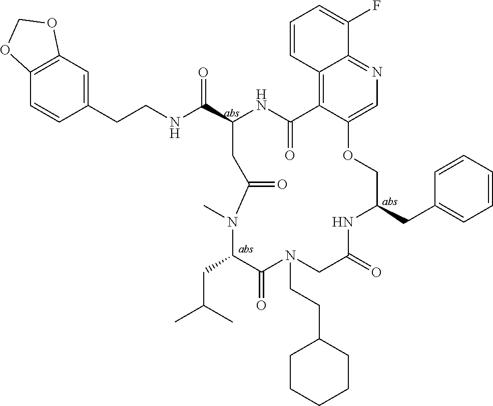 [SMILES: CC(C)C[C@H]1C(=O)N(CCC2CCCCC2)CC(=O)N[C@H](Cc2ccccc2)COc2cnc3c(F)cccc3c2C(=O)N[C@H](C(=O)NCCc2ccc3c(c2)OCO3)CC(=O)N1C] | 382 |
| 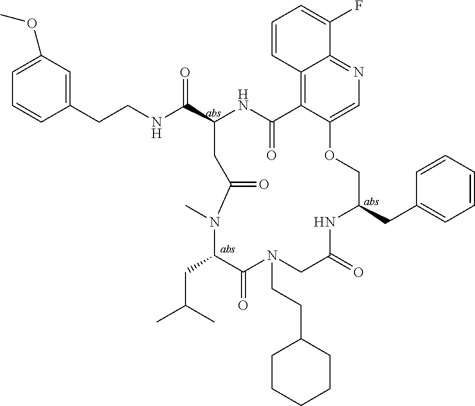 [SMILES: COc1cccc(CCNC(=O)[C@@H]2CC(=O)N(C)[C@@H](CC(C)C)C(=O)N(CCC3CCCCC3)CC(=O)N[C@H](Cc3ccccc3)COc3cnc4c(F)cccc4c3C(=O)N2)c1] | 383 |
| 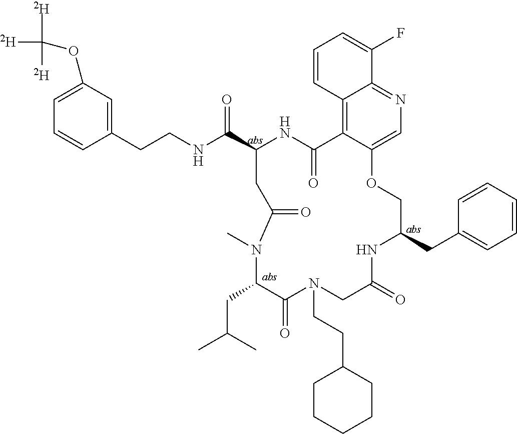 [SMILES: [2H]C([2H])([2H])Oc1cccc(CCNC(=O)[C@@H]2CC(=O)N(C)[C@@H](CC(C)C)C(=O)N(CCC3CCCCC3)CC(=O)N[C@H](Cc3ccccc3)COc3cnc4c(F)cccc4c3C(=O)N2)c1] | 384 |
| 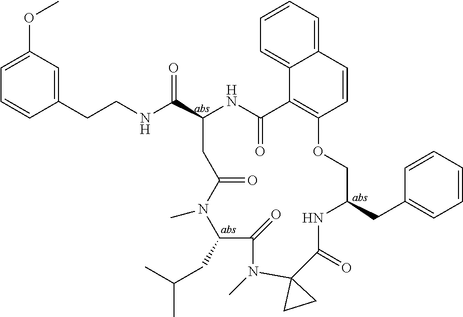 [SMILES: COc1cccc(CCNC(=O)[C@@H]2CC(=O)N(C)[C@@H](CC(C)C)C(=O)N(C)C3(CC3)C(=O)N[C@H](Cc3ccccc3)COc3ccc4ccccc4c3C(=O)N2)c1] | 385 |
| 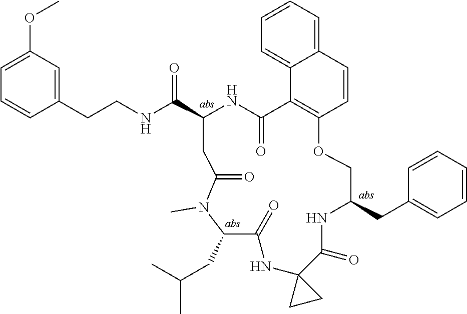 [SMILES: COc1cccc(CCNC(=O)[C@@H]2CC(=O)N(C)[C@@H](CC(C)C)C(=O)NC3(CC3)C(=O)N[C@H](Cc3ccccc3)COc3ccc4ccccc4c3C(=O)N2)c1] | 386 |
| 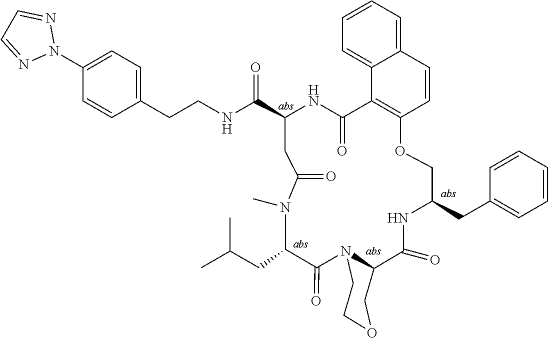 | 387 |
| 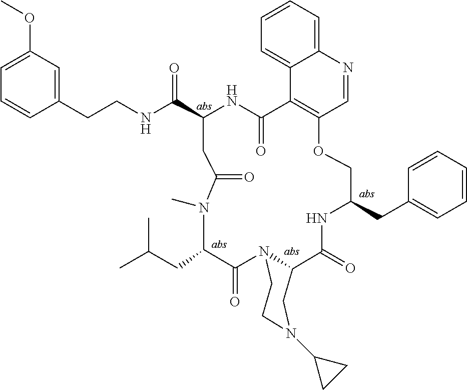 | 388 |
| 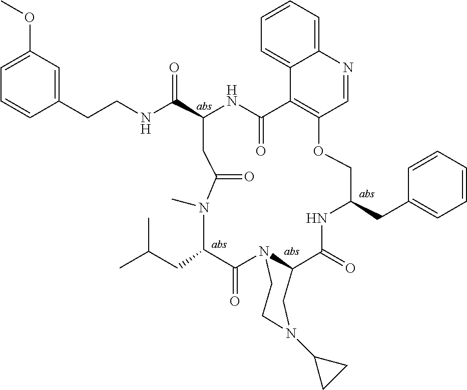 | 389 |
| 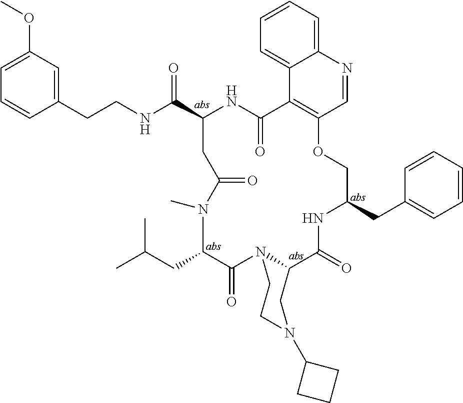 | 390 |
| 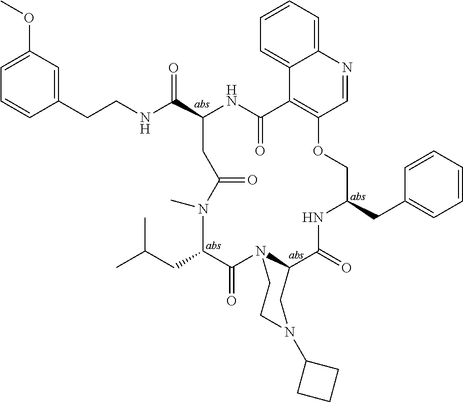 | 391 |
| 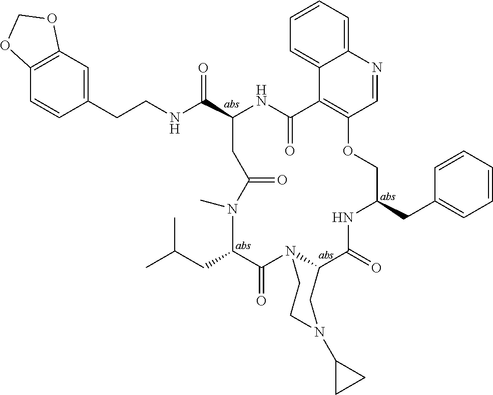 | 392 |
| 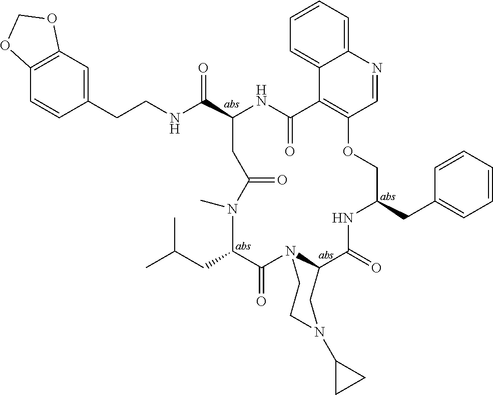 | 393 |
| 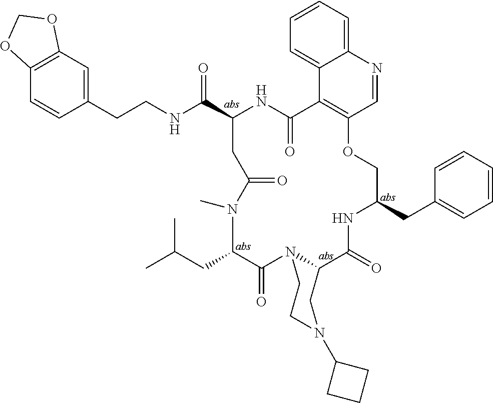 | 394 |
| 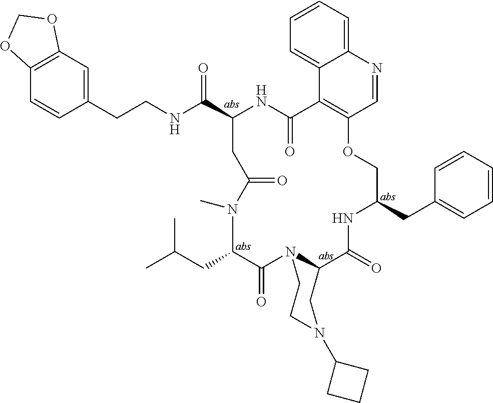 | 395 |
| 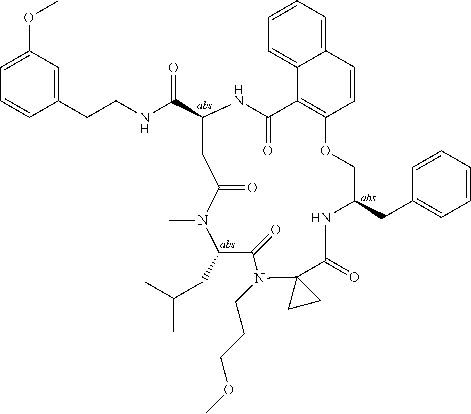 [SMILES: COCCCN1C(=O)[C@H](CC(C)C)N(C)C(=O)C[C@@H](C(=O)NCCc2cccc(OC)c2)NC(=O)c2c(ccc3ccccc23)OC[C@@H](Cc2ccccc2)NC(=O)C12CC2] | 396 |
| 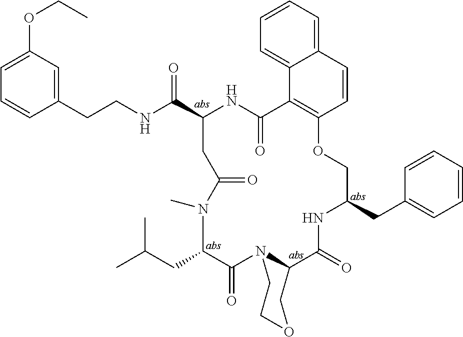 | 397 |
| 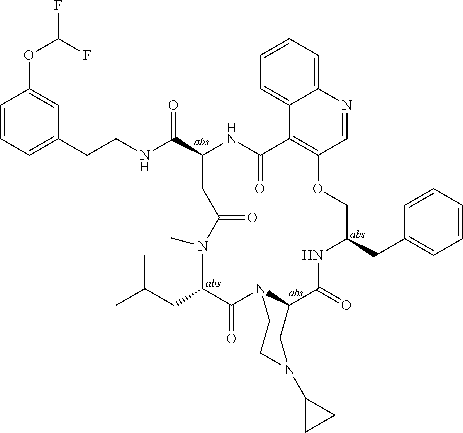 | 398 |
| 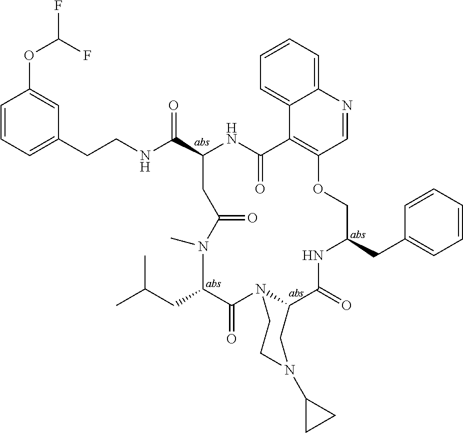 | 399 |
| 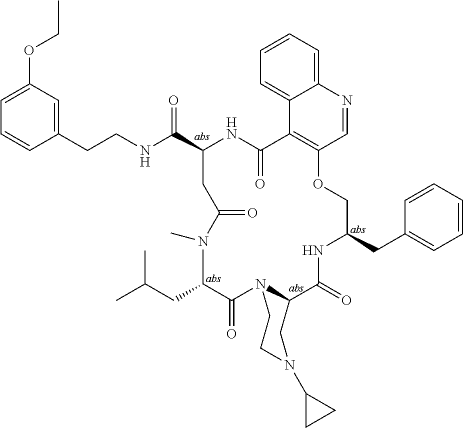 | 400 |
| 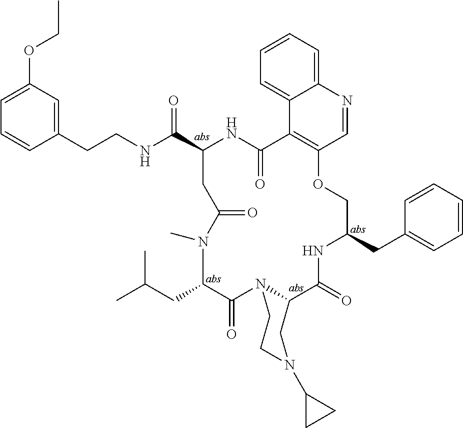 | 401 |
| 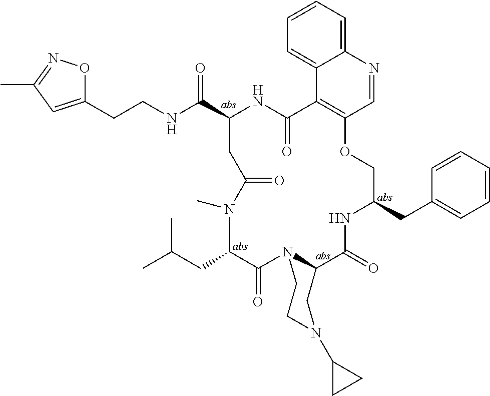 | 402 |
| 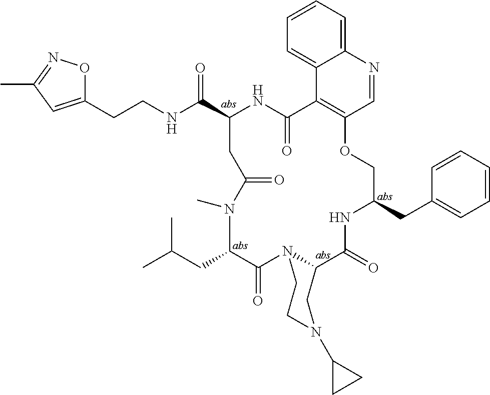 | 403 |
| 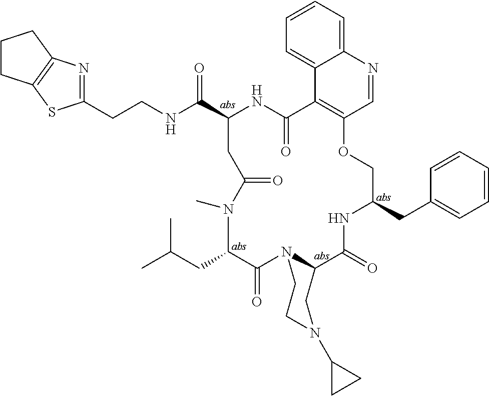 | 404 |
| 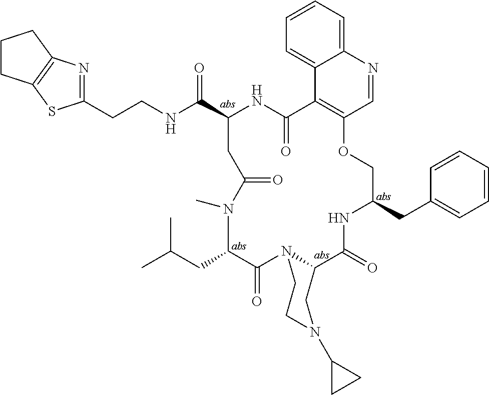 | 405 |
| 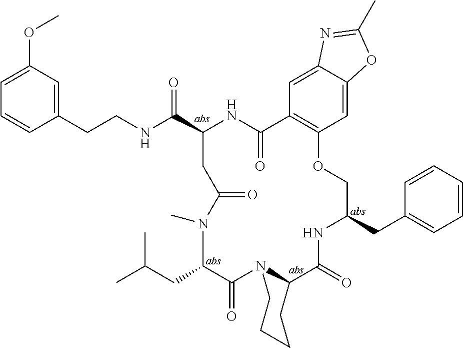 | 406 |
| 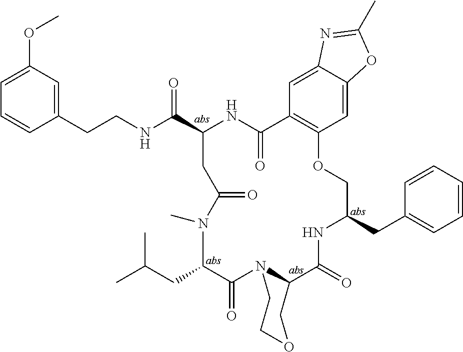 | 407 |
| 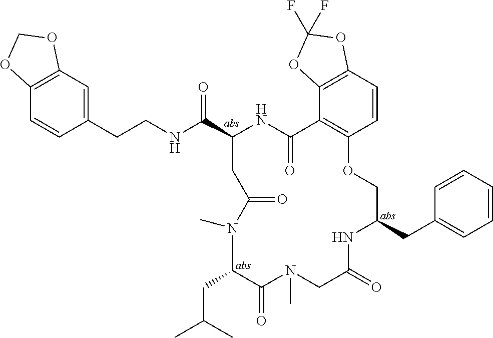 [SMILES: CC(C)C[C@H]1C(=O)N(C)CC(=O)N[C@H](Cc2ccccc2)COc2ccc3c(c2C(=O)N[C@H](C(=O)NCCc2ccc4c(c2)OCO4)CC(=O)N1C)OC(F)(F)O3] | 408 |
| 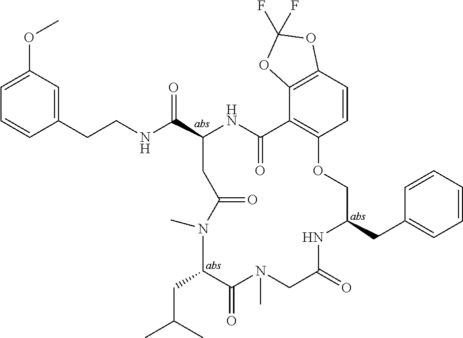 [SMILES: COc1cccc(CCNC(=O)[C@@H]2CC(=O)N(C)[C@@H](CC(C)C)C(=O)N(C)CC(=O)N[C@H](Cc3ccccc3)COc3ccc4c(c3C(=O)N2)OC(F)(F)O4)c1] | 409 |
| 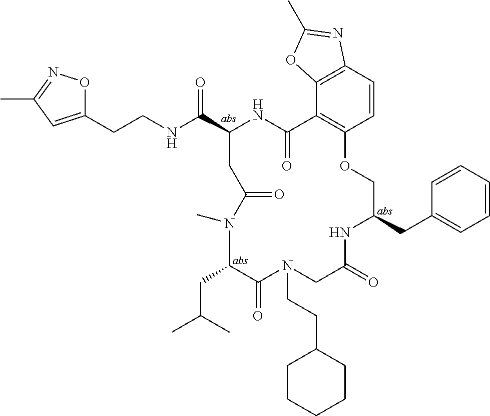 [SMILES: Cc1cc(CCNC(=O)[C@@H]2CC(=O)N(C)[C@@H](CC(C)C)C(=O)N(CCC3CCCCC3)CC(=O)N[C@H](Cc3ccccc3)COc3ccc4nc(C)oc4c3C(=O)N2)on1] | 410 |
| 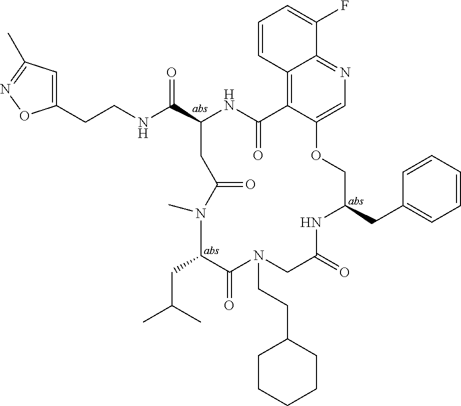 [SMILES: Cc1cc(CCNC(=O)[C@@H]2CC(=O)N(C)[C@@H](CC(C)C)C(=O)N(CCC3CCCCC3)CC(=O)N[C@H](Cc3ccccc3)COc3cnc4c(F)cccc4c3C(=O)N2)on1] | 411 |
| 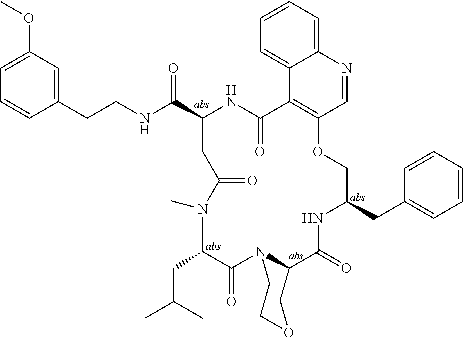 | 412 |
| 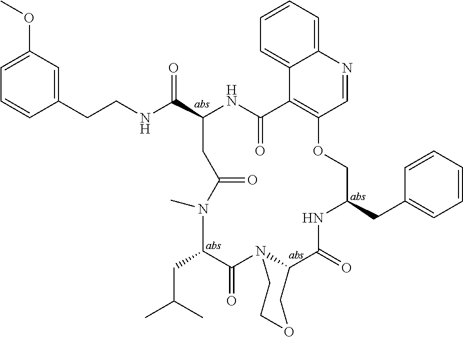 | 413 |
| 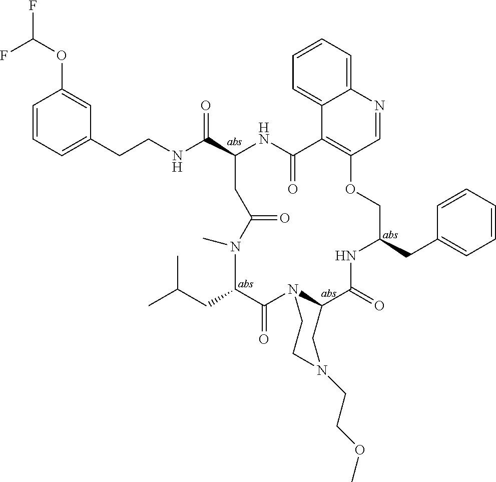 | 414 |
| 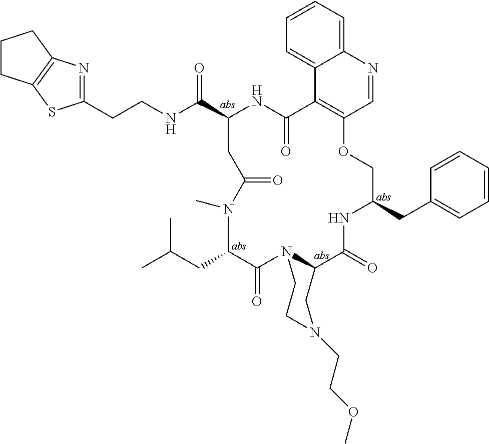 | 415 |
| 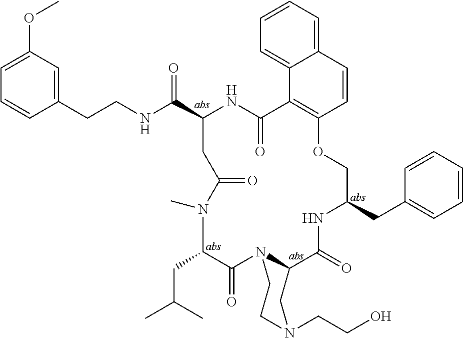 | 416 |
| 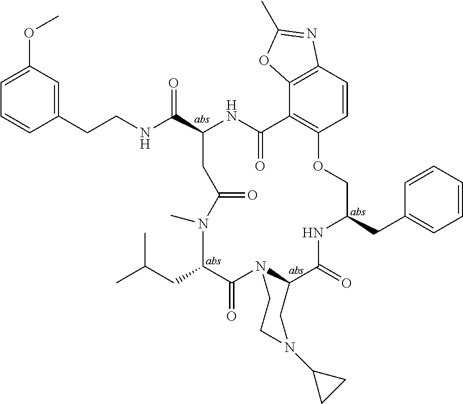 | 417 |
| 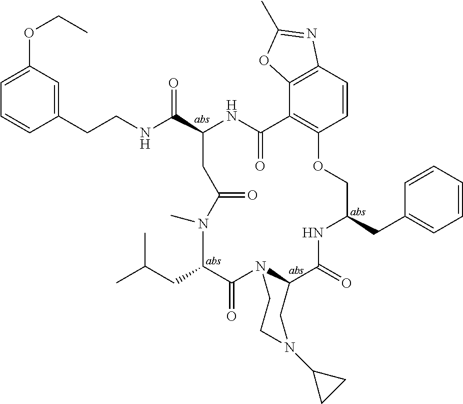 | 418 |
| 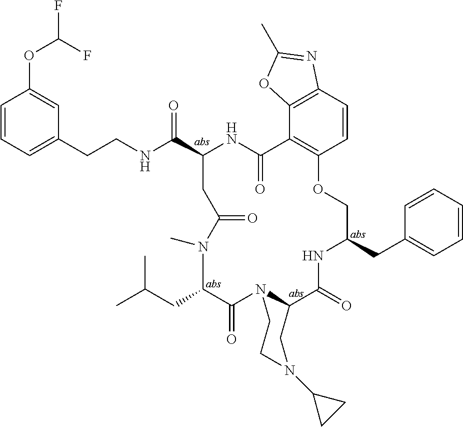 | 419 |
| 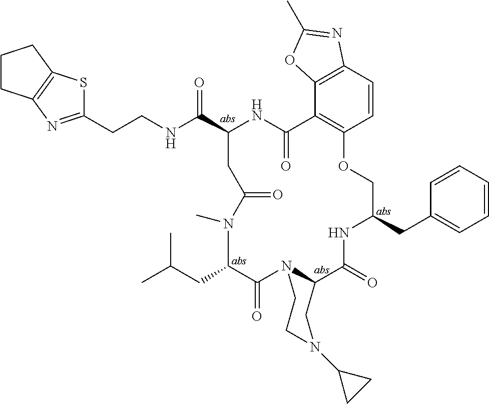 | 420 |
| 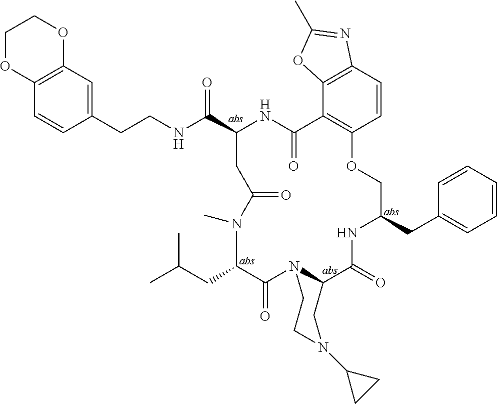 | 421 |
| 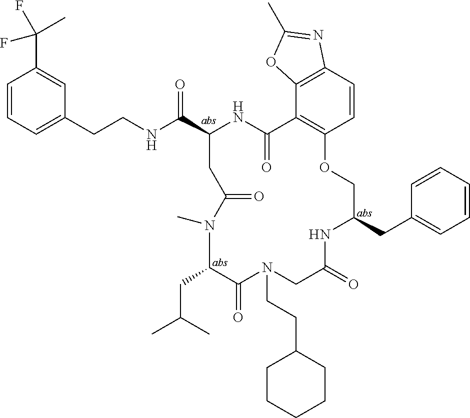 [SMILES: Cc1nc2ccc3c(c2o1)C(=O)N[C@H](C(=O)NCCc1cccc(C(C)(F)F)c1)CC(=O)N(C)[C@@H](CC(C)C)C(=O)N(CCC1CCCCC1)CC(=O)N[C@H](Cc1ccccc1)CO3] | 422 |
| 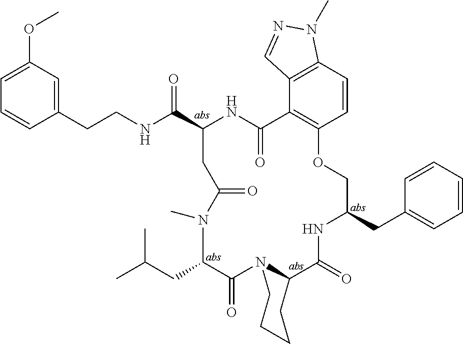 | 423 |
| 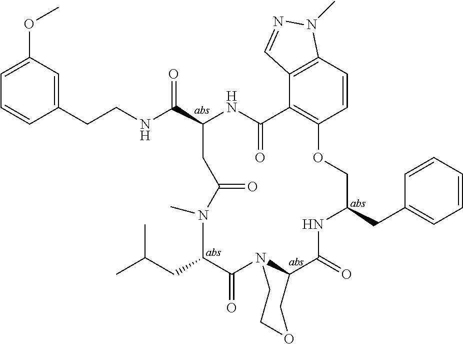 | 424 |
| 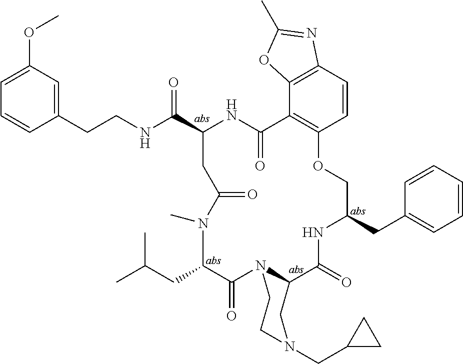 | 425 |
| 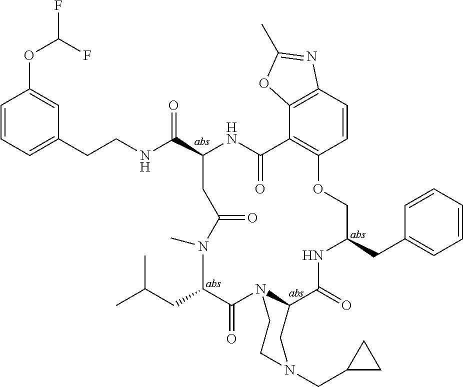 | 426 |
| 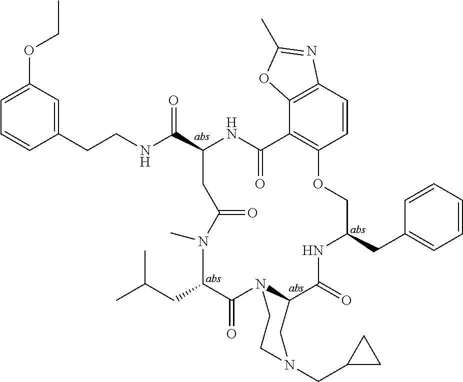 | 427 |
| 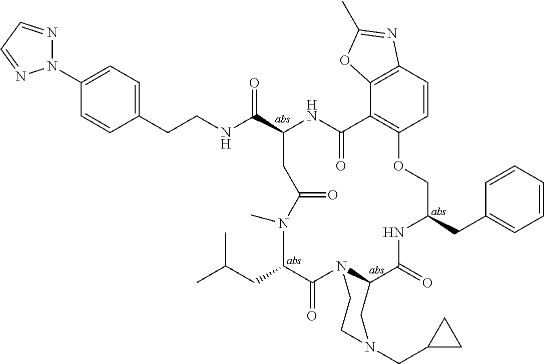 | 428 |
| 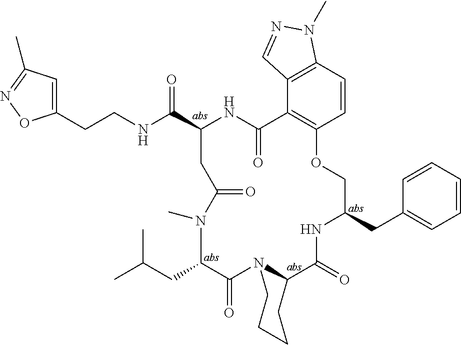 | 429 |
| 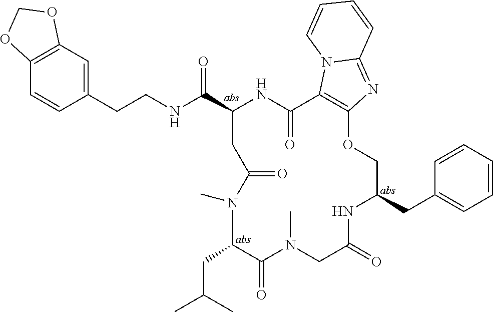 [SMILES: CC(C)C[C@H]1C(=O)N(C)CC(=O)N[C@H](Cc2ccccc2)COc2nc3ccccn3c2C(=O)N[C@H](C(=O)NCCc2ccc3c(c2)OCO3)CC(=O)N1C] | 430 |
| 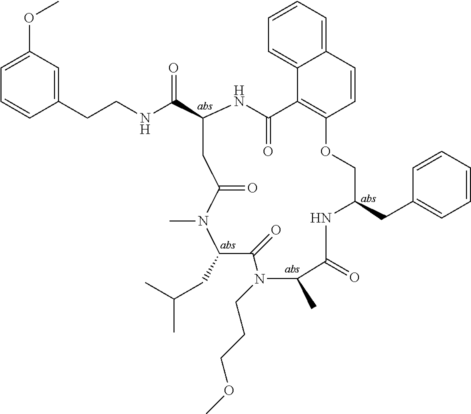 [SMILES: COCCCN1C(=O)[C@H](CC(C)C)N(C)C(=O)C[C@@H](C(=O)NCCc2cccc(OC)c2)NC(=O)c2c(ccc3ccccc23)OC[C@@H](Cc2ccccc2)NC(=O)[C@H]1C] | 431 |
| 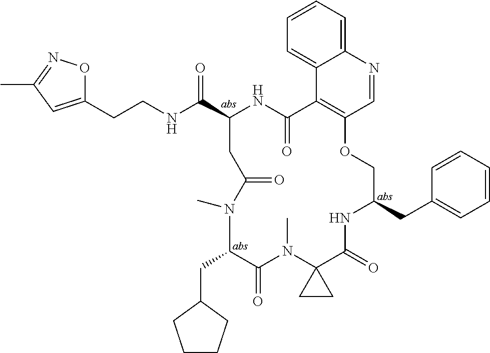 [SMILES: Cc1cc(CCNC(=O)[C@@H]2CC(=O)N(C)[C@@H](CC3CCCC3)C(=O)N(C)C3(CC3)C(=O)N[C@H](Cc3ccccc3)COc3cnc4ccccc4c3C(=O)N2)on1] | 432 |
| 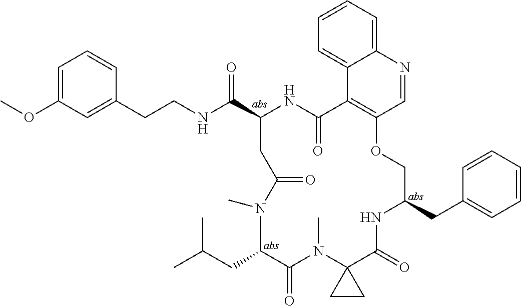 [SMILES: COc1cccc(CCNC(=O)[C@@H]2CC(=O)N(C)[C@@H](CC(C)C)C(=O)N(C)C3(CC3)C(=O)N[C@H](Cc3ccccc3)COc3cnc4ccccc4c3C(=O)N2)c1] | 433 |
| 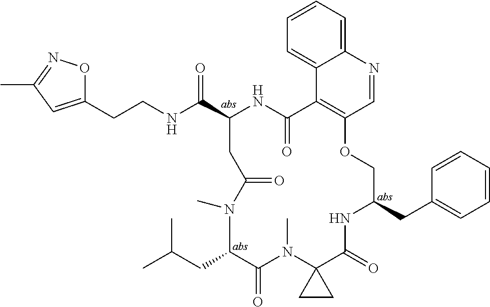 [SMILES: Cc1cc(CCNC(=O)[C@@H]2CC(=O)N(C)[C@@H](CC(C)C)C(=O)N(C)C3(CC3)C(=O)N[C@H](Cc3ccccc3)COc3cnc4ccccc4c3C(=O)N2)on1] | 434 |
| 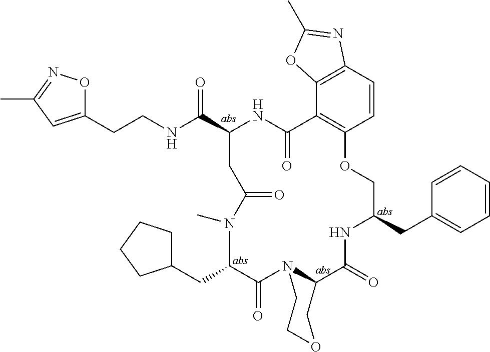 | 435 |
| 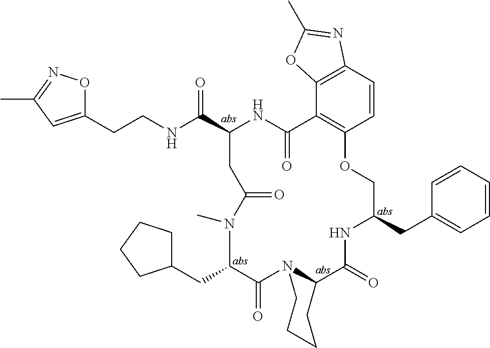 | 436 |
| 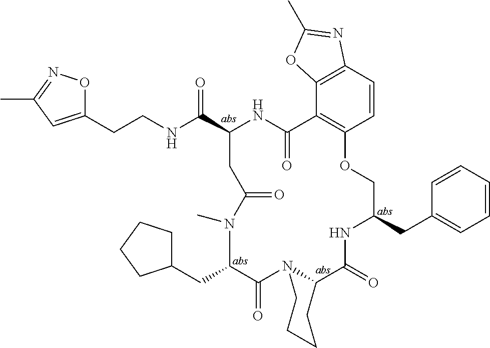 | 437 |
| 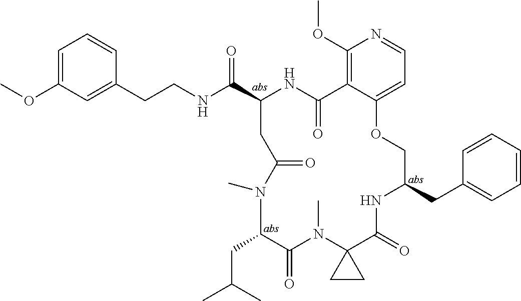 [SMILES: COc1cccc(CCNC(=O)[C@@H]2CC(=O)N(C)[C@@H](CC(C)C)C(=O)N(C)C3(CC3)C(=O)N[C@H](Cc3ccccc3)COc3ccnc(OC)c3C(=O)N2)c1] | 438 |
| 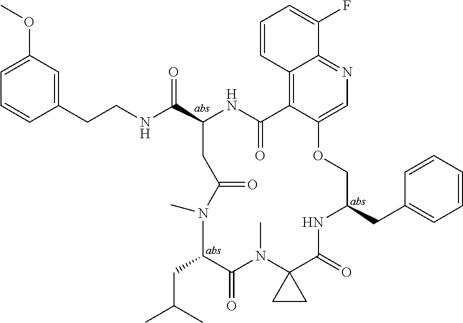 [SMILES: COc1cccc(CCNC(=O)[C@@H]2CC(=O)N(C)[C@@H](CC(C)C)C(=O)N(C)C3(CC3)C(=O)N[C@H](Cc3ccccc3)COc3cnc4c(F)cccc4c3C(=O)N2)c1] | 439 |
| 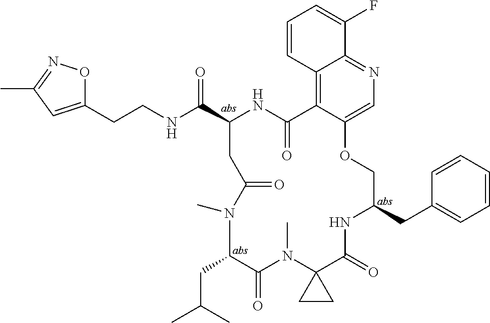 [SMILES: Cc1cc(CCNC(=O)[C@@H]2CC(=O)N(C)[C@@H](CC(C)C)C(=O)N(C)C3(CC3)C(=O)N[C@H](Cc3ccccc3)COc3cnc4c(F)cccc4c3C(=O)N2)on1] | 440 |
| 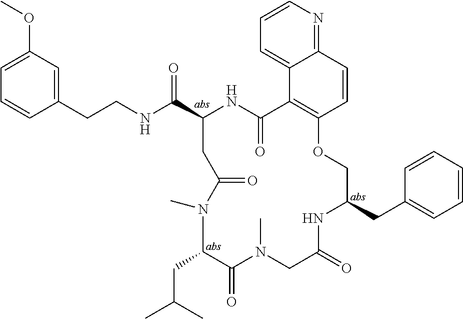 [SMILES: COc1cccc(CCNC(=O)[C@@H]2CC(=O)N(C)[C@@H](CC(C)C)C(=O)N(C)CC(=O)N[C@H](Cc3ccccc3)COc3ccc4ncccc4c3C(=O)N2)c1] | 441 |
| 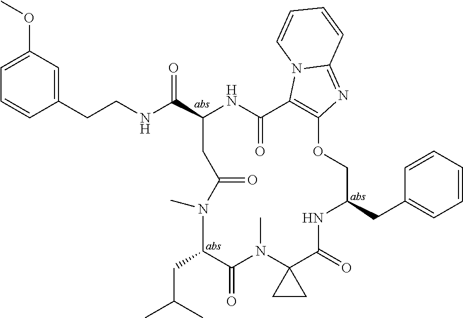 [SMILES: COc1cccc(CCNC(=O)[C@@H]2CC(=O)N(C)[C@@H](CC(C)C)C(=O)N(C)C3(CC3)C(=O)N[C@H](Cc3ccccc3)COc3nc4ccccn4c3C(=O)N2)c1] | 442 |
| 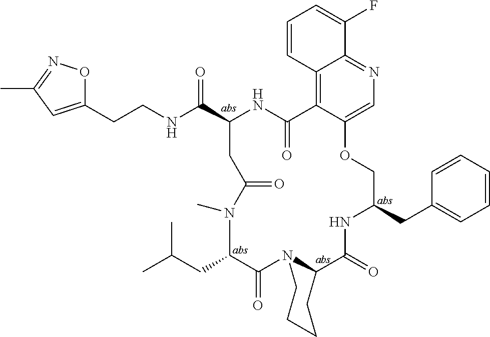 | 443 |
| 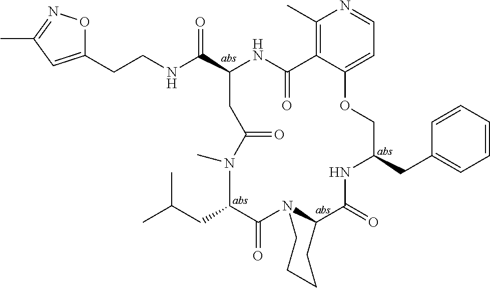 | 444 |
| 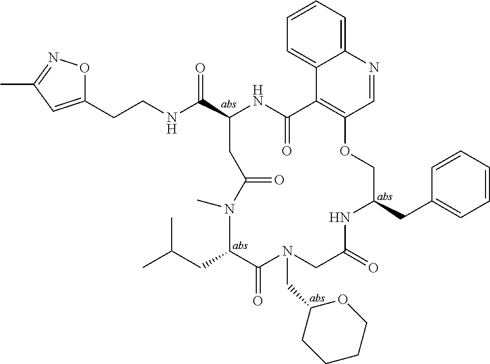 [SMILES: Cc1cc(CCNC(=O)[C@@H]2CC(=O)N(C)[C@@H](CC(C)C)C(=O)N(C[C@H]3CCCCO3)CC(=O)N[C@H](Cc3ccccc3)COc3cnc4ccccc4c3C(=O)N2)on1] | 445 |
| 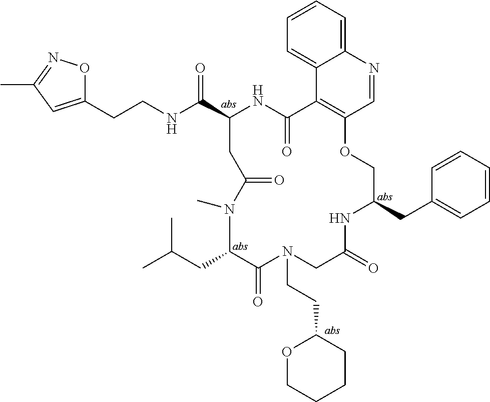 [SMILES: Cc1cc(CCNC(=O)[C@@H]2CC(=O)N(C)[C@@H](CC(C)C)C(=O)N(CC[C@@H]3CCCCO3)CC(=O)N[C@H](Cc3ccccc3)COc3cnc4ccccc4c3C(=O)N2)on1] | 446 |
| 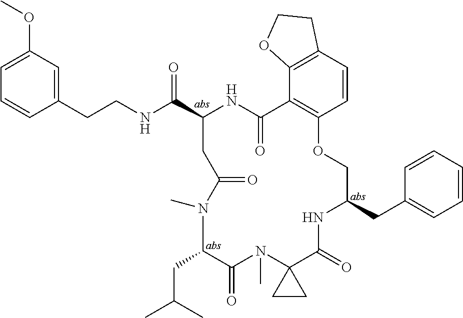 [SMILES: COc1cccc(CCNC(=O)[C@@H]2CC(=O)N(C)[C@@H](CC(C)C)C(=O)N(C)C3(CC3)C(=O)N[C@H](Cc3ccccc3)COc3ccc4c(c3C(=O)N2)OCC4)c1] | 447 |
| 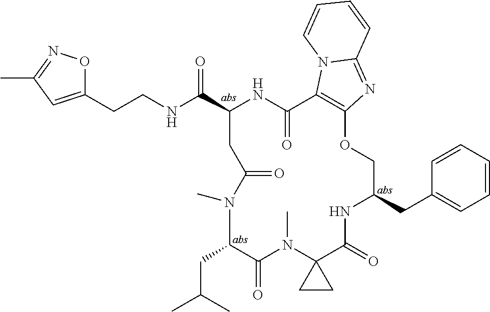 [SMILES: Cc1cc(CCNC(=O)[C@@H]2CC(=O)N(C)[C@@H](CC(C)C)C(=O)N(C)C3(CC3)C(=O)N[C@H](Cc3ccccc3)COc3nc4ccccn4c3C(=O)N2)on1] | 448 |
| 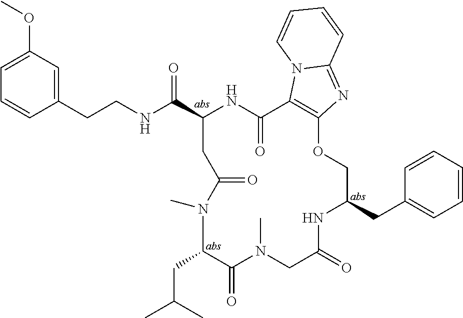 [SMILES: COc1cccc(CCNC(=O)[C@@H]2CC(=O)N(C)[C@@H](CC(C)C)C(=O)N(C)CC(=O)N[C@H](Cc3ccccc3)COc3nc4ccccn4c3C(=O)N2)c1] | 449 |
| 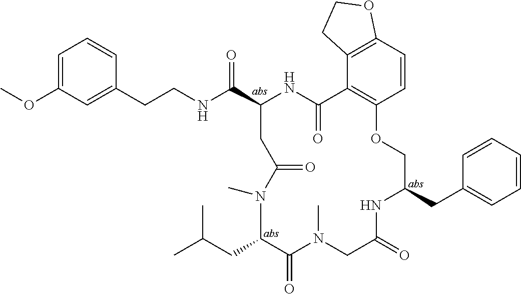 [SMILES: COc1cccc(CCNC(=O)[C@@H]2CC(=O)N(C)[C@@H](CC(C)C)C(=O)N(C)CC(=O)N[C@H](Cc3ccccc3)COc3ccc4c(c3C(=O)N2)CCO4)c1] | 450 |
| 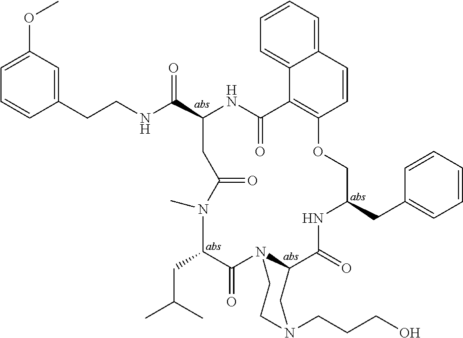 | 451 |
| 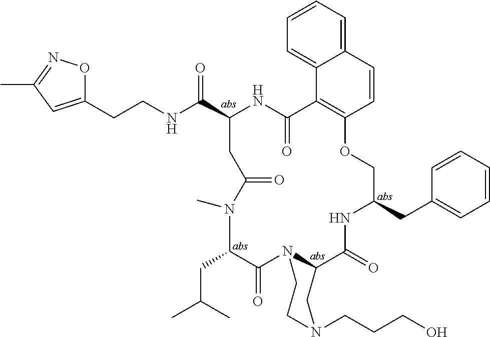 | 452 |
| 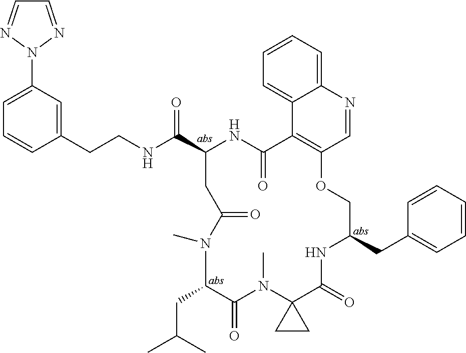 [SMILES: CC(C)C[C@H]1C(=O)N(C)C2(CC2)C(=O)N[C@H](Cc2ccccc2)COc2cnc3ccccc3c2C(=O)N[C@H](C(=O)NCCc2cccc(-n3nccn3)c2)CC(=O)N1C] | 453 |
| 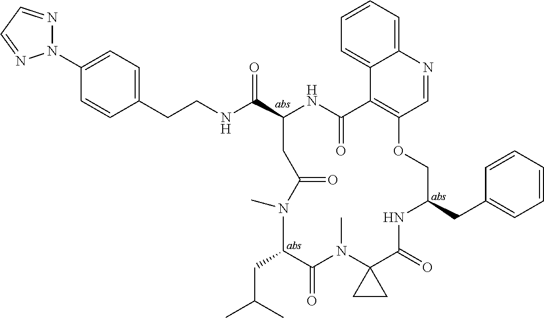 [SMILES: CC(C)C[C@H]1C(=O)N(C)C2(CC2)C(=O)N[C@H](Cc2ccccc2)COc2cnc3ccccc3c2C(=O)N[C@H](C(=O)NCCc2ccc(-n3nccn3)cc2)CC(=O)N1C] | 454 |
| 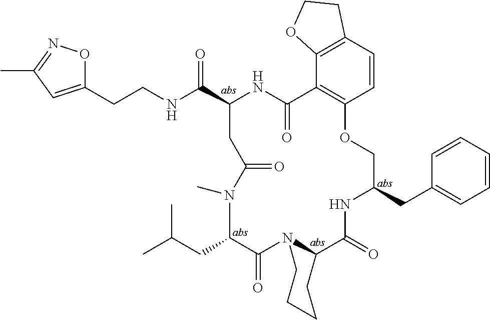 | 455 |
| 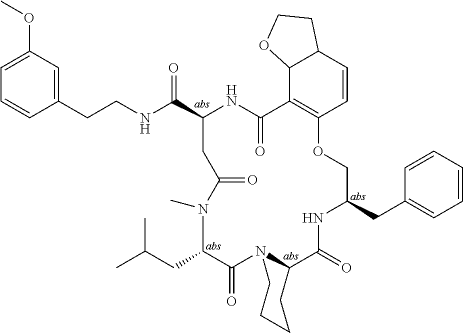 | 456 |
| 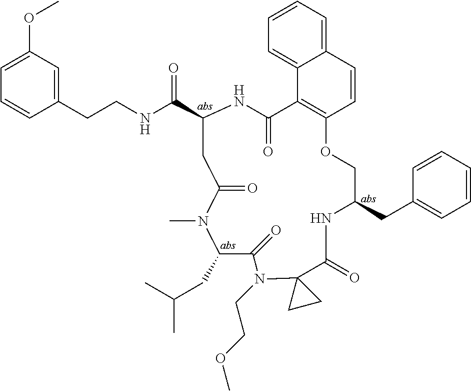 [SMILES: COCCN1C(=O)[C@H](CC(C)C)N(C)C(=O)C[C@@H](C(=O)NCCc2cccc(OC)c2)NC(=O)c2c(ccc3ccccc23)OC[C@@H](Cc2ccccc2)NC(=O)C12CC2] | 457 |
| 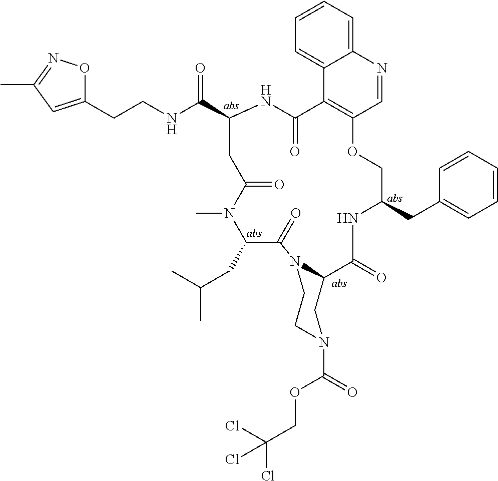 | 458 |
| 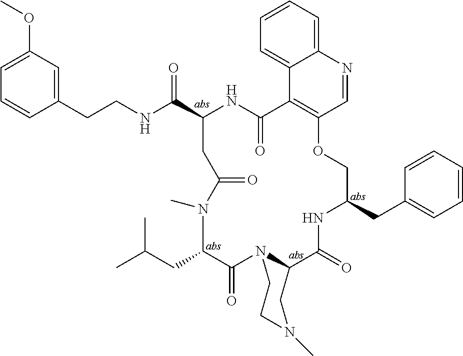 | 459 |
| 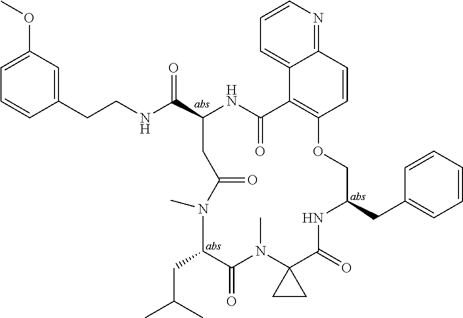 [SMILES: COc1cccc(CCNC(=O)[C@@H]2CC(=O)N(C)[C@@H](CC(C)C)C(=O)N(C)C3(CC3)C(=O)N[C@H](Cc3ccccc3)COc3ccc4ncccc4c3C(=O)N2)c1] | 460 |
| 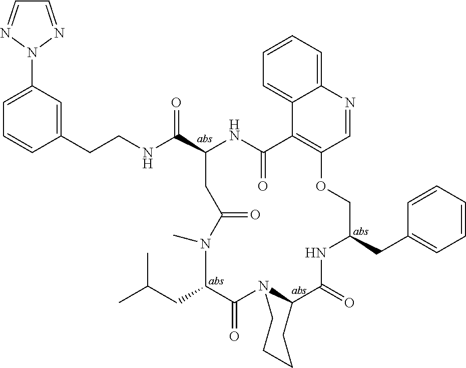 | 461 |
| 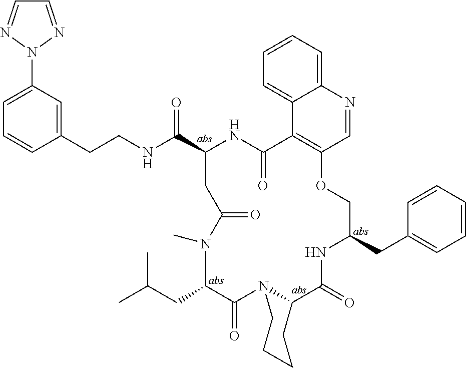 | 462 |
| 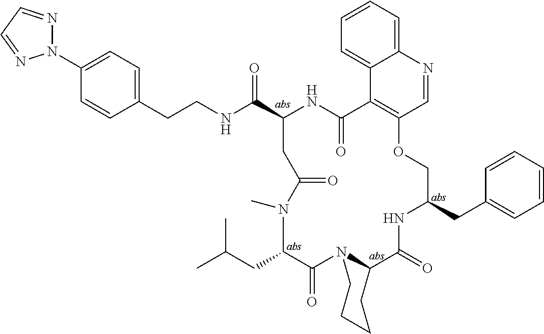 | 463 |
| 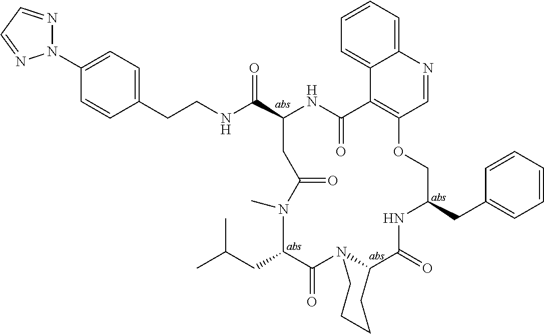 | 464 |
| 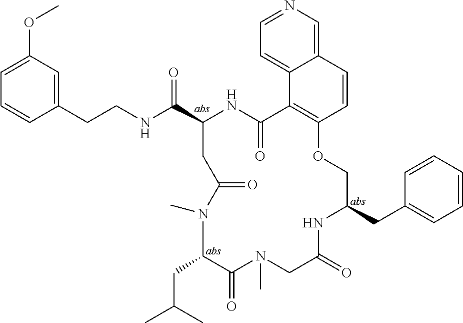 [SMILES: COc1cccc(CCNC(=O)[C@@H]2CC(=O)N(C)[C@@H](CC(C)C)C(=O)N(C)CC(=O)N[C@H](Cc3ccccc3)COc3ccc4cnccc4c3C(=O)N2)c1] | 465 |
| 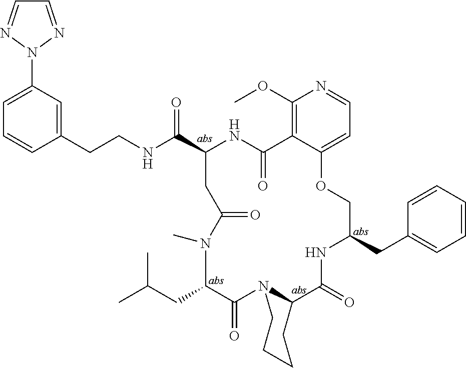 | 466 |
| 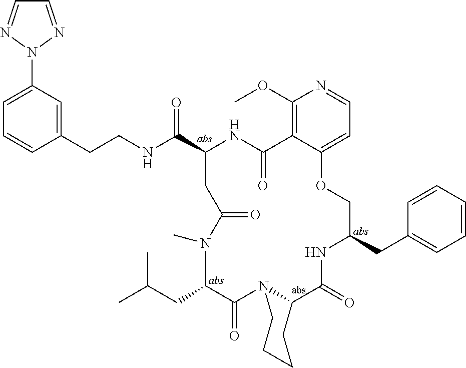 | 467 |
| 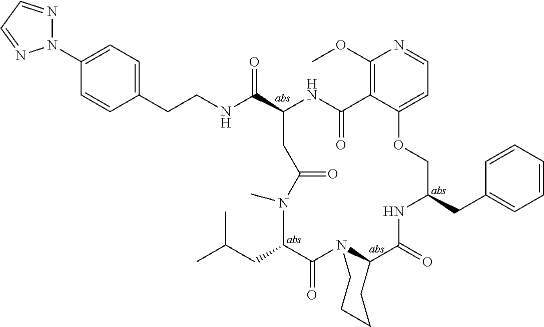 | 468 |
| 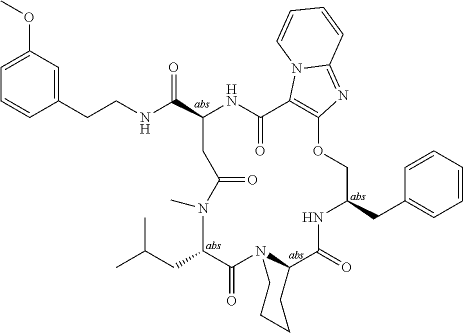 | 469 |
| 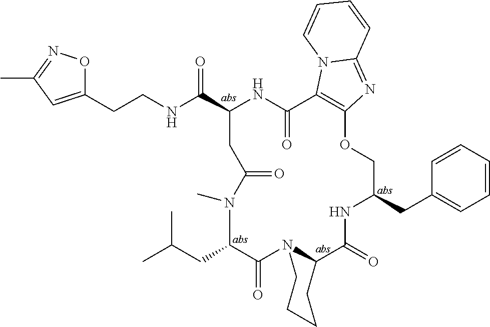 | 470 |
| 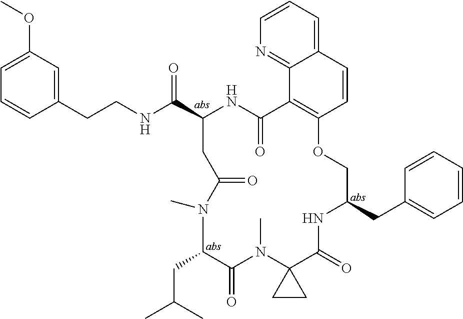 [SMILES: COc1cccc(CCNC(=O)[C@@H]2CC(=O)N(C)[C@@H](CC(C)C)C(=O)N(C)C3(CC3)C(=O)N[C@H](Cc3ccccc3)COc3ccc4cccnc4c3C(=O)N2)c1] | 471 |
| 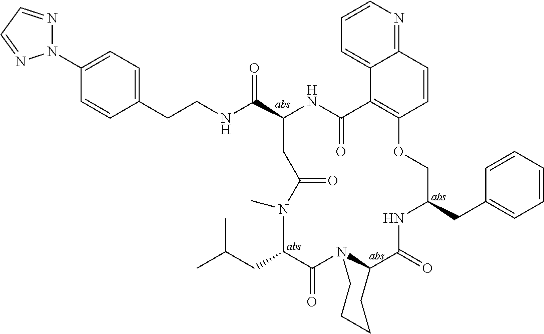 | 472 |
| 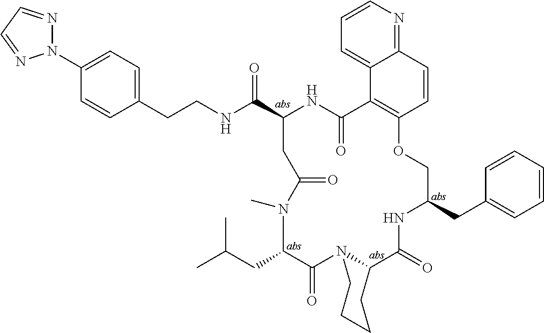 | 473 |
| 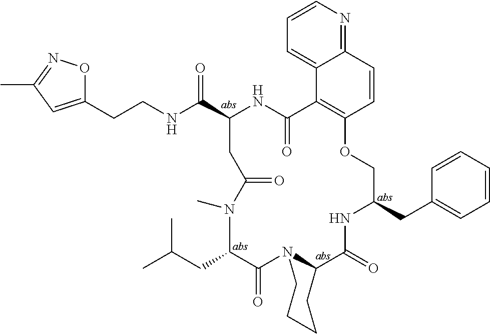 | 474 |
| 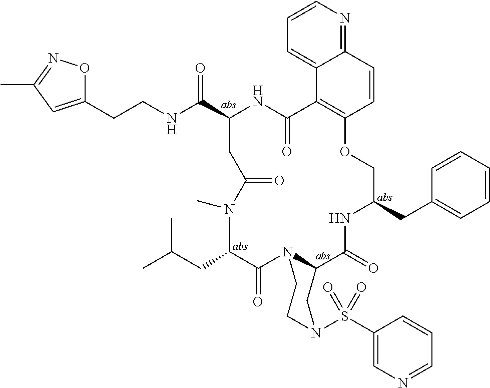 | 475 |
| 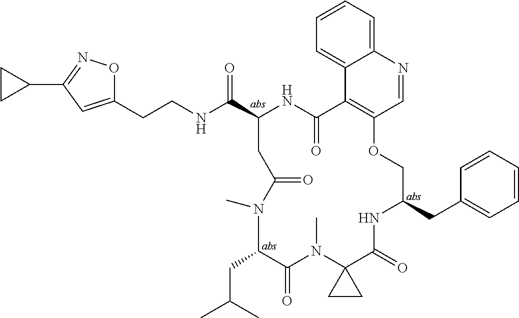 [SMILES: CC(C)C[C@H]1C(=O)N(C)C2(CC2)C(=O)N[C@H](Cc2ccccc2)COc2cnc3ccccc3c2C(=O)N[C@H](C(=O)NCCc2cc(C3CC3)no2)CC(=O)N1C] | 476 |
| 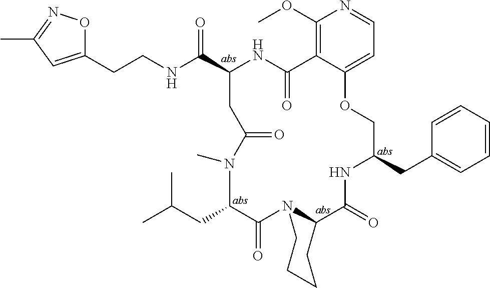 | 477 |
| 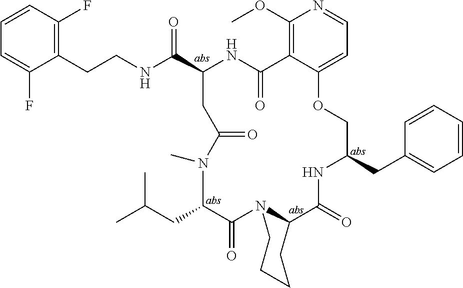 | 478 |
| 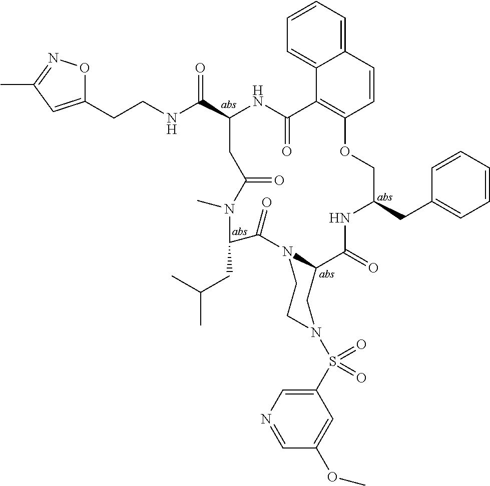 | 479 |
| 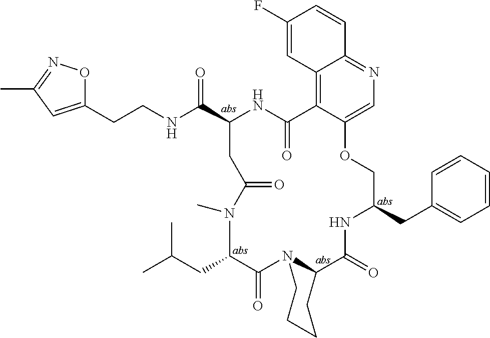 | 480 |
| 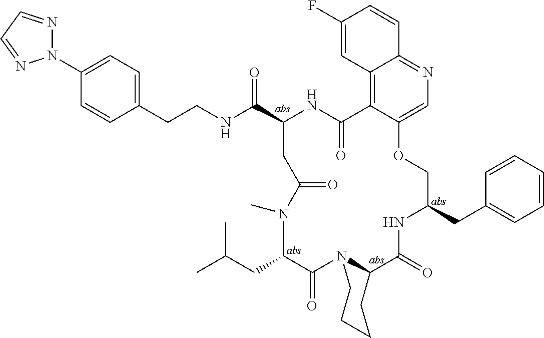 | 481 |
| 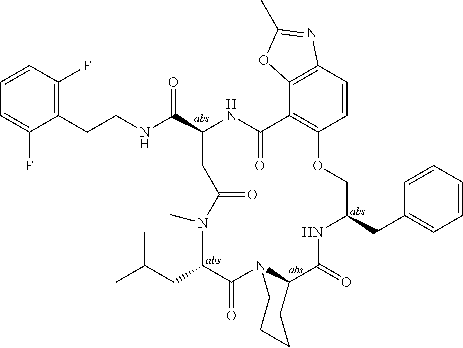 | 482 |
| 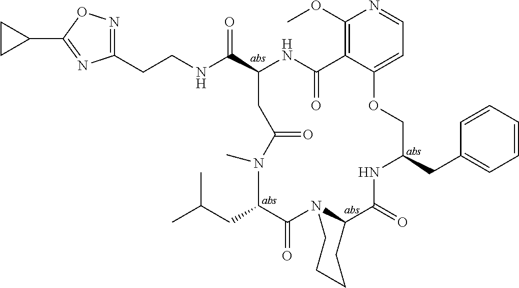 | 483 |
| 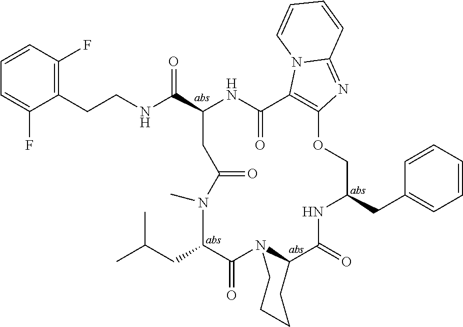 | 484 |
| 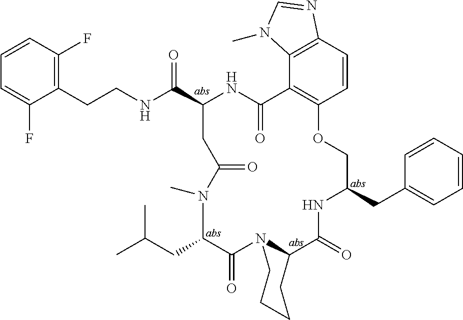 | 485 |
| 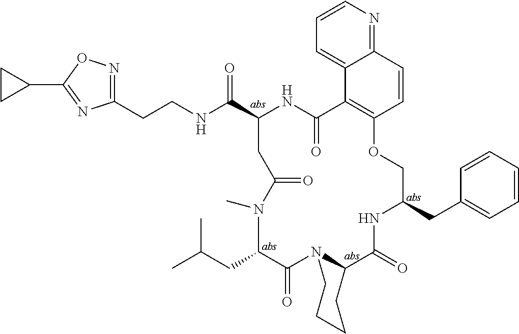 | 486 |
| 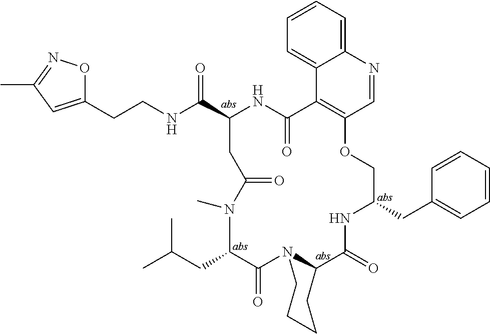 | 487 |
| 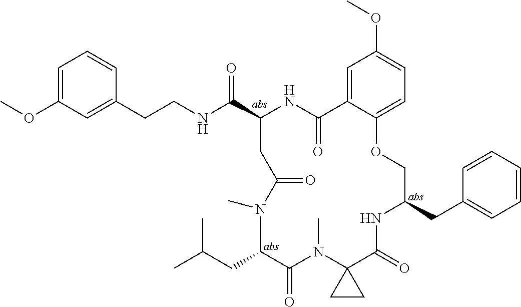 [SMILES: COc1cccc(CCNC(=O)[C@@H]2CC(=O)N(C)[C@@H](CC(C)C)C(=O)N(C)C3(CC3)C(=O)N[C@H](Cc3ccccc3)COc3ccc(OC)cc3C(=O)N2)c1] | 488 |
| 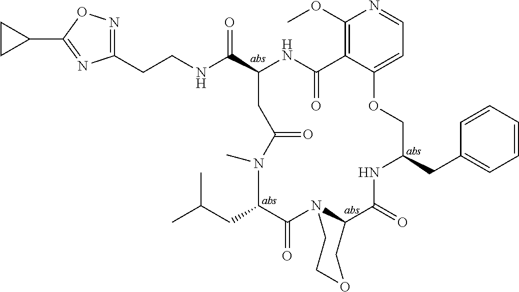 | 489 |
| 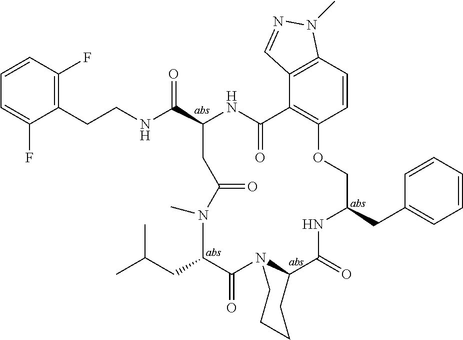 | 490 |
| 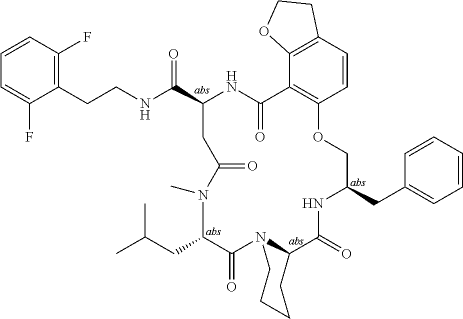 | 491 |
| 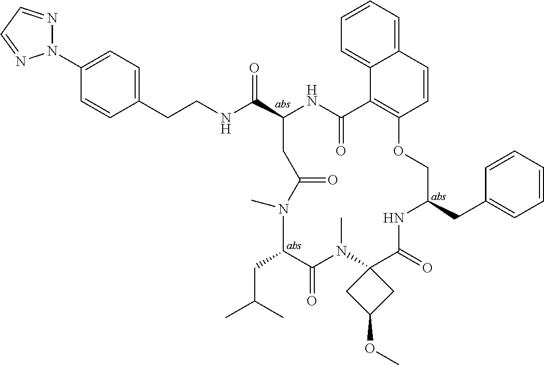 [SMILES: CO[C@H]1C[C@]2(C1)C(=O)N[C@H](Cc1ccccc1)COc1ccc3ccccc3c1C(=O)N[C@H](C(=O)NCCc1ccc(-n3nccn3)cc1)CC(=O)N(C)[C@@H](CC(C)C)C(=O)N2C] | 492 |
| 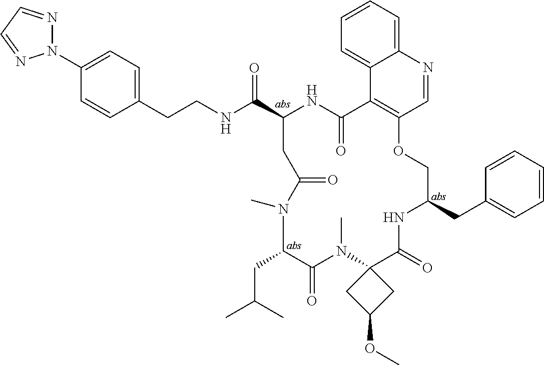 [SMILES: CO[C@H]1C[C@]2(C1)C(=O)N[C@H](Cc1ccccc1)COc1cnc3ccccc3c1C(=O)N[C@H](C(=O)NCCc1ccc(-n3nccn3)cc1)CC(=O)N(C)[C@@H](CC(C)C)C(=O)N2C] | 493 |
| 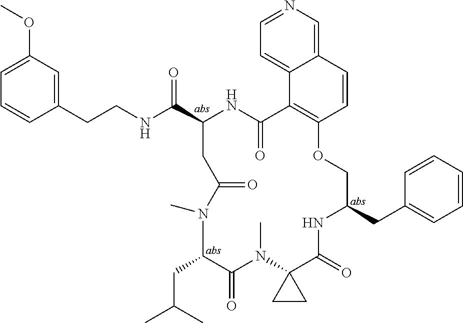 | 494 |
| 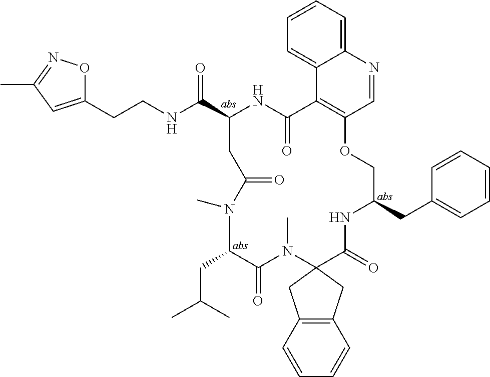 [SMILES: Cc1cc(CCNC(=O)[C@@H]2CC(=O)N(C)[C@@H](CC(C)C)C(=O)N(C)C3(Cc4ccccc4C3)C(=O)N[C@H](Cc3ccccc3)COc3cnc4ccccc4c3C(=O)N2)on1] | 495 |
| 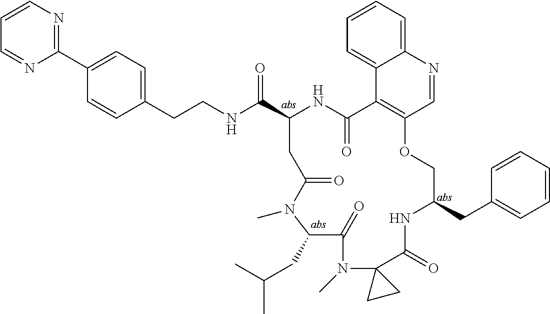 [SMILES: CC(C)C[C@H]1C(=O)N(C)C2(CC2)C(=O)N[C@H](Cc2ccccc2)COc2cnc3ccccc3c2C(=O)N[C@H](C(=O)NCCc2ccc(-c3ncccn3)cc2)CC(=O)N1C] | 496 |
| 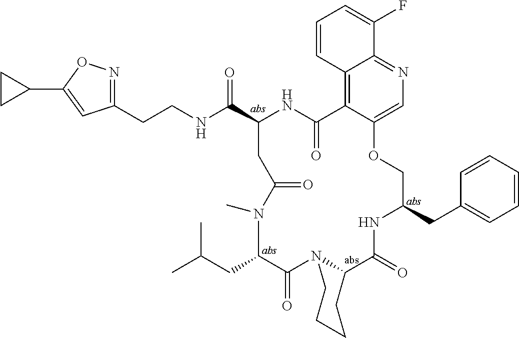 | 497 |
| 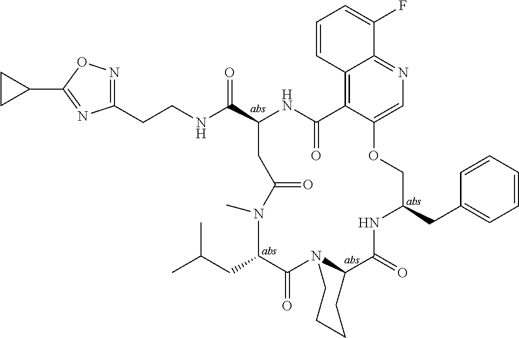 | 498 |
| 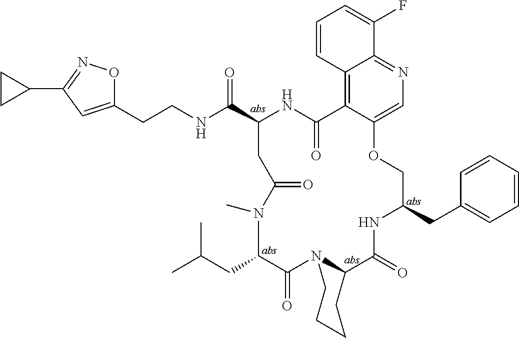 | 499 |
| 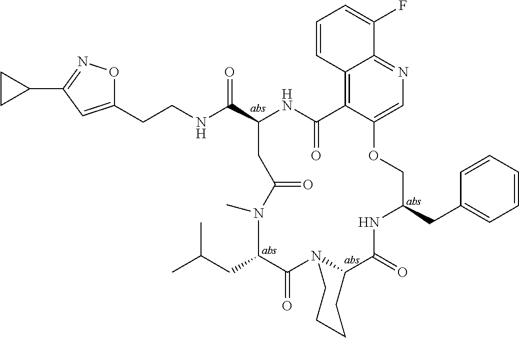 | 500 |
| 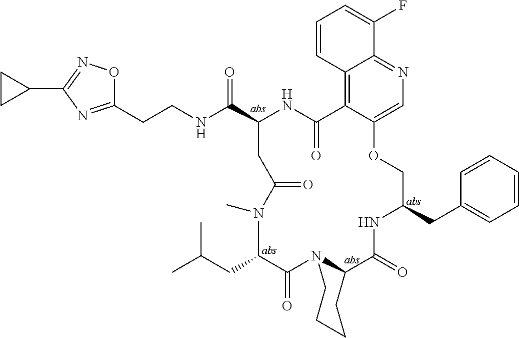 | 501 |
| 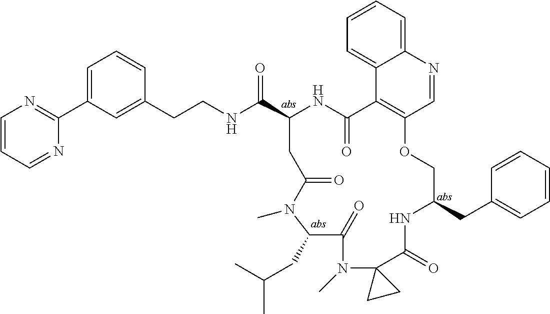 [SMILES: CC(C)C[C@H]1C(=O)N(C)C2(CC2)C(=O)N[C@H](Cc2ccccc2)COc2cnc3ccccc3c2C(=O)N[C@H](C(=O)NCCc2cccc(-c3ncccn3)c2)CC(=O)N1C] | 502 |
| 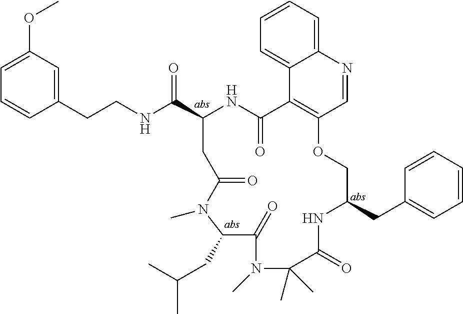 [SMILES: COc1cccc(CCNC(=O)[C@@H]2CC(=O)N(C)[C@@H](CC(C)C)C(=O)N(C)C(C)(C)C(=O)N[C@H](Cc3ccccc3)COc3cnc4ccccc4c3C(=O)N2)c1] | 503 |
| 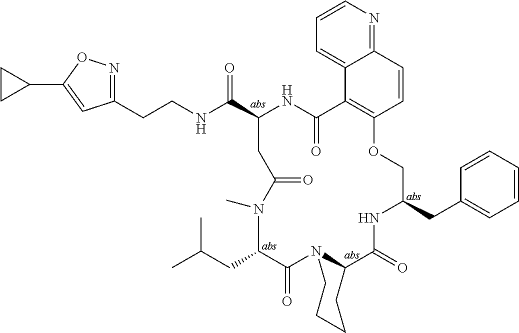 | 504 |
| 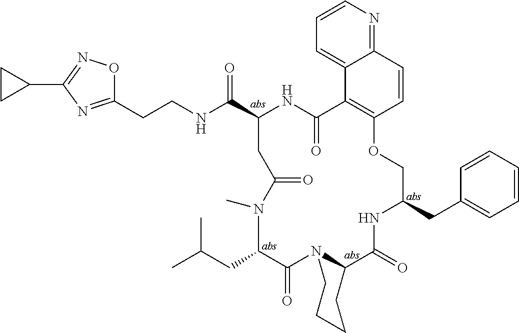 | 505 |
| 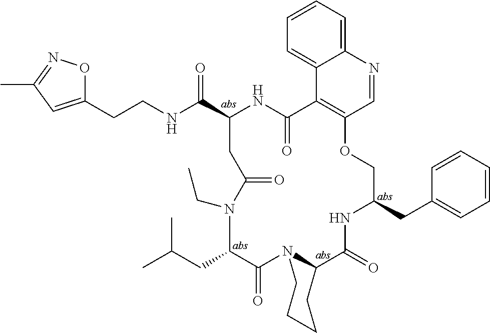 | 506 |
| 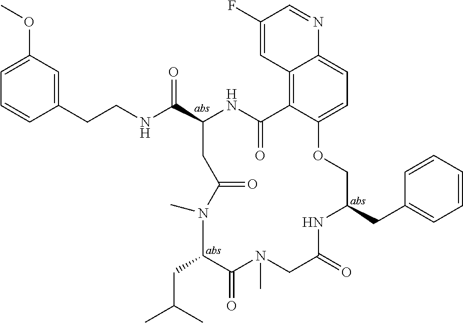 [SMILES: COc1cccc(CCNC(=O)[C@@H]2CC(=O)N(C)[C@@H](CC(C)C)C(=O)N(C)CC(=O)N[C@H](Cc3ccccc3)COc3ccc4ncc(F)cc4c3C(=O)N2)c1] | 507 |
| 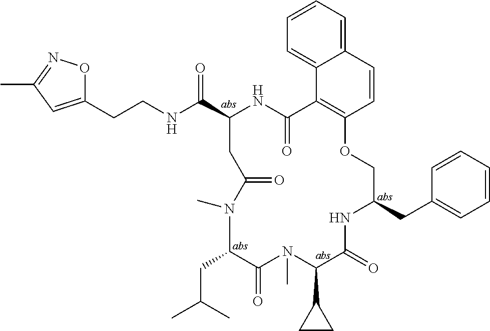 [SMILES: Cc1cc(CCNC(=O)[C@@H]2CC(=O)N(C)[C@@H](CC(C)C)C(=O)N(C)[C@H](C3CC3)C(=O)N[C@H](Cc3ccccc3)COc3ccc4ccccc4c3C(=O)N2)on1] | 508 |
| 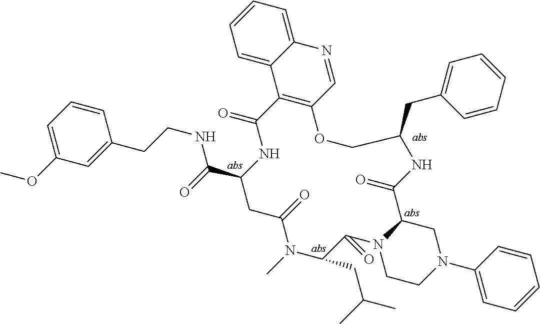 [SMILES: COc1cccc(CCNC(=O)[C@@H]2CC(=O)N(C)[C@@H](CC(C)C)C(=O)N3CCN(c4ccccc4)C[C@@H]3C(=O)N[C@H](Cc3ccccc3)COc3cnc4ccccc4c3C(=O)N2)c1] | 509 |
| 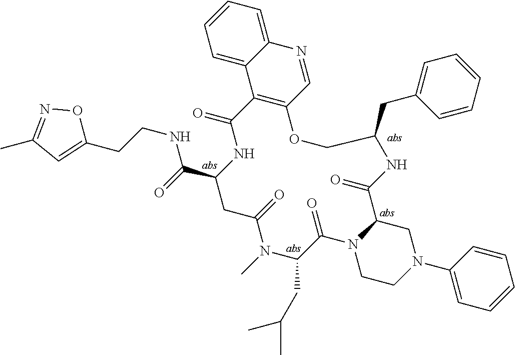 [SMILES: Cc1cc(CCNC(=O)[C@@H]2CC(=O)N(C)[C@@H](CC(C)C)C(=O)N3CCN(c4ccccc4)C[C@@H]3C(=O)N[C@H](Cc3ccccc3)COc3cnc4ccccc4c3C(=O)N2)on1] | 510 |
| 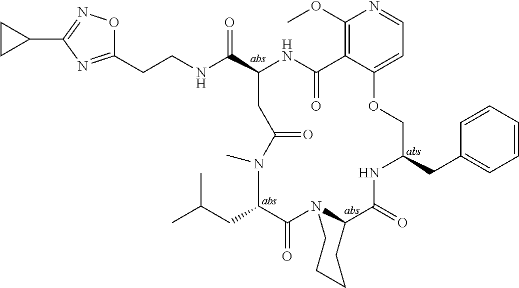 | 511 |
| 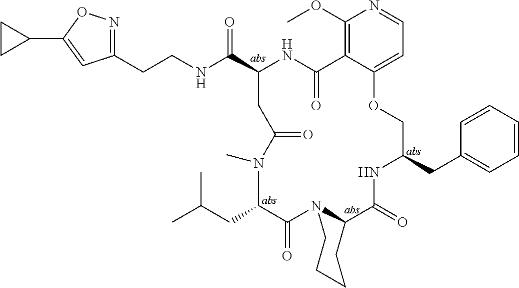 | 512 |
| 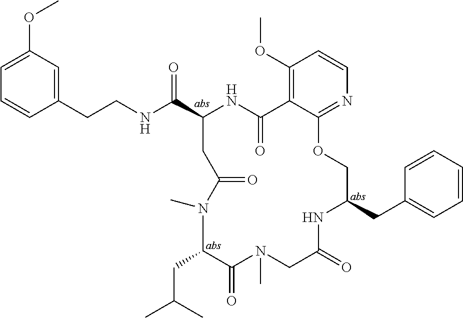 [SMILES: COc1cccc(CCNC(=O)[C@@H]2CC(=O)N(C)[C@@H](CC(C)C)C(=O)N(C)CC(=O)N[C@H](Cc3ccccc3)COc3nccc(OC)c3C(=O)N2)c1] | 513 |
| 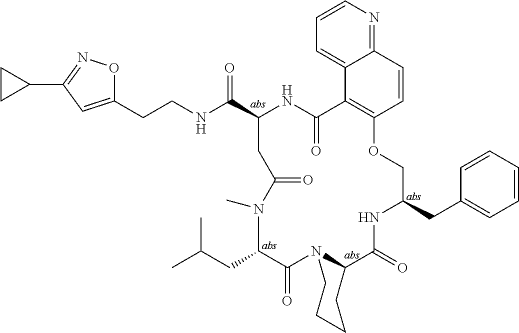 | 514 |
| 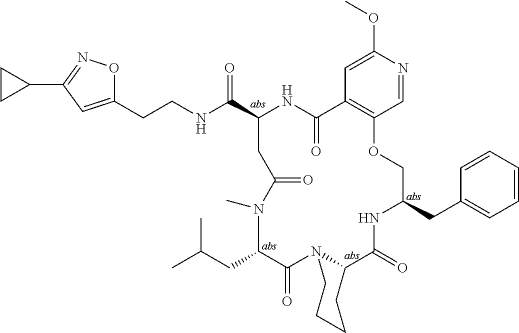 | 515 |
| 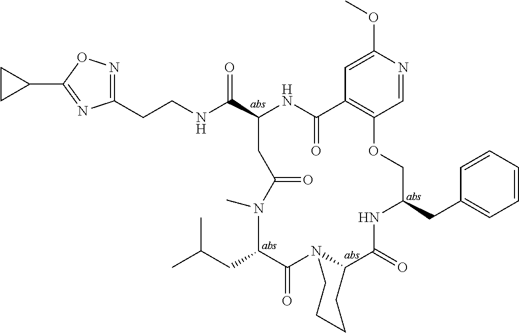 | 516 |
| 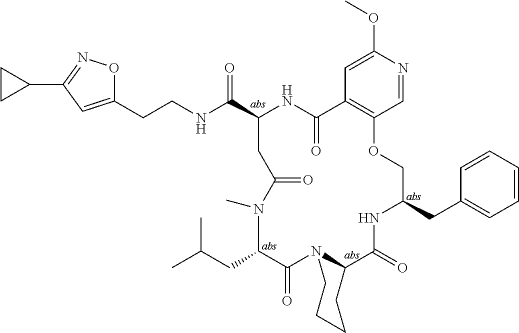 | 517 |
| 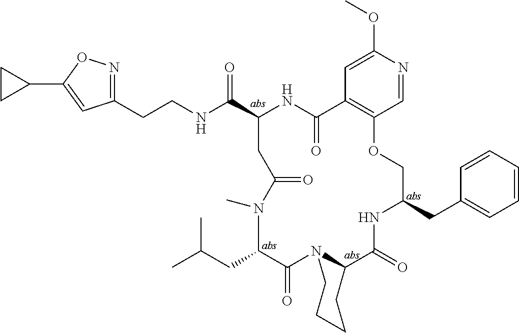 | 518 |
| 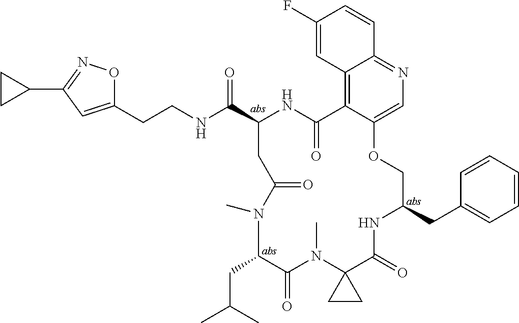 [SMILES: CC(C)C[C@H]1C(=O)N(C)C2(CC2)C(=O)N[C@H](Cc2ccccc2)COc2cnc3ccc(F)cc3c2C(=O)N[C@H](C(=O)NCCc2cc(C3CC3)no2)CC(=O)N1C] | 519 |
| 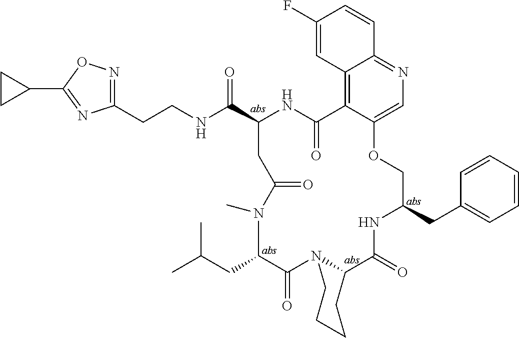 | 520 |
| 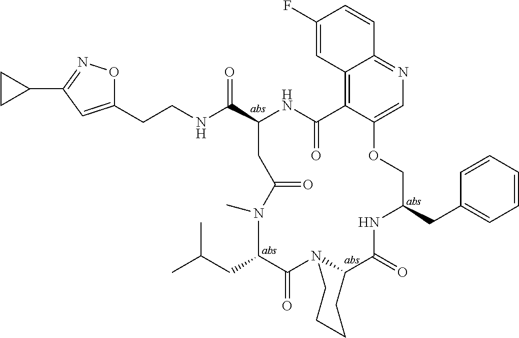 | 521 |
| 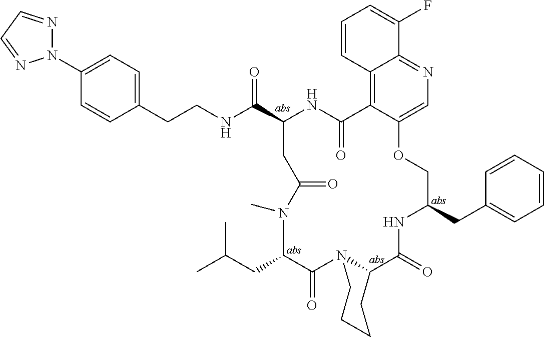 | 522 |
| 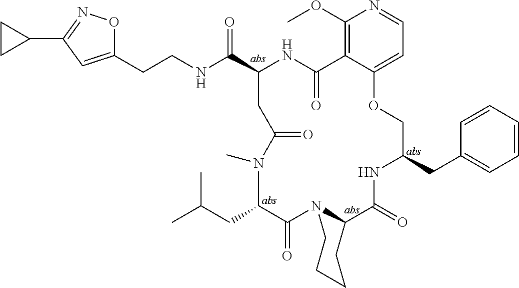 | 523 |
| 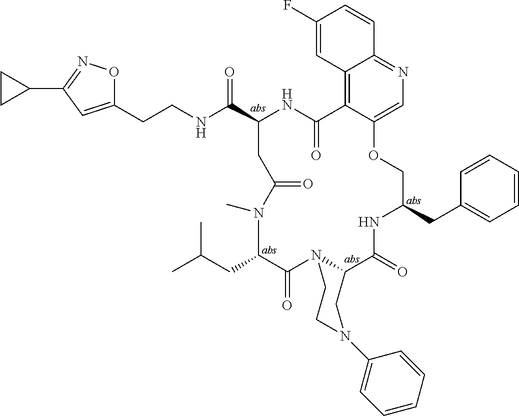 | 524 |
| 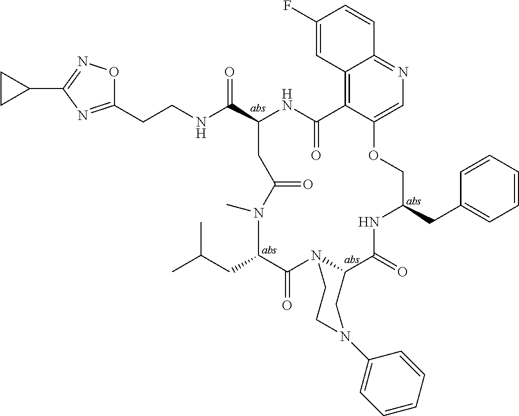 | 525 |
| 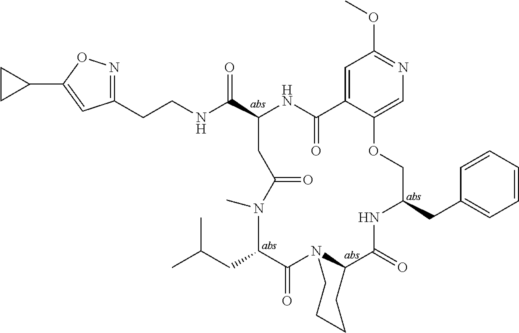 | 526 |
| 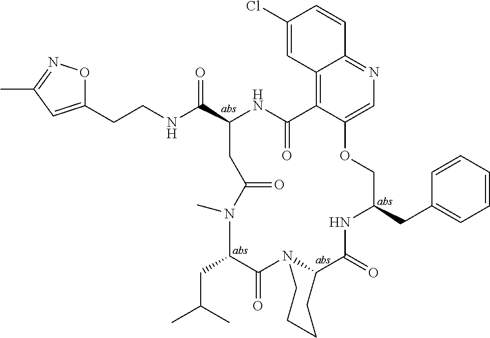 | 527 |
| 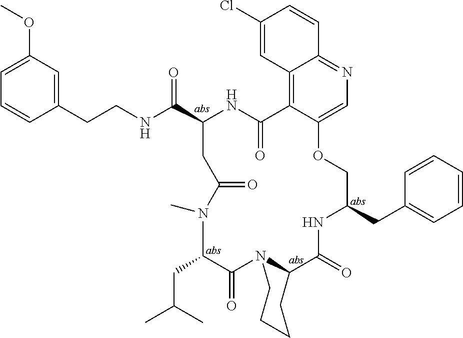 | 528 |
| 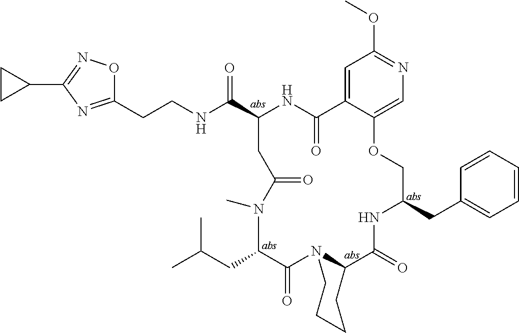 | 529 |
| 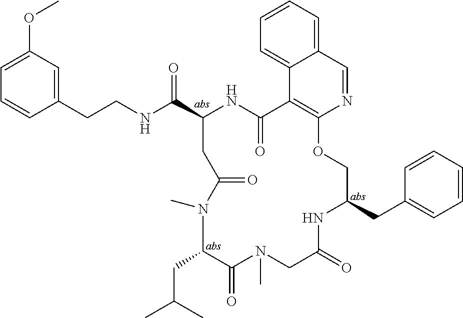 [SMILES: COc1cccc(CCNC(=O)[C@@H]2CC(=O)N(C)[C@@H](CC(C)C)C(=O)N(C)CC(=O)N[C@H](Cc3ccccc3)COc3ncc4ccccc4c3C(=O)N2)c1] | 530 |
| 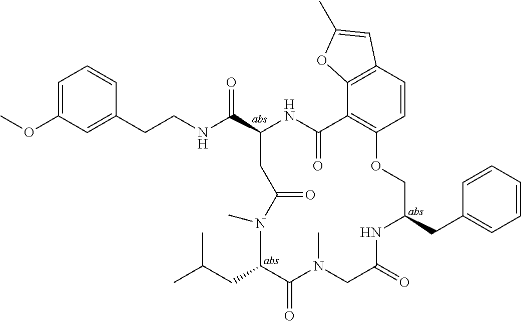 [SMILES: COc1cccc(CCNC(=O)[C@@H]2CC(=O)N(C)[C@@H](CC(C)C)C(=O)N(C)CC(=O)N[C@H](Cc3ccccc3)COc3ccc4cc(C)oc4c3C(=O)N2)c1] | 531 |
| 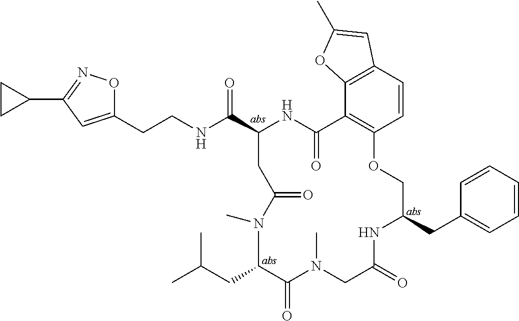 [SMILES: Cc1cc2ccc3c(c2o1)C(=O)N[C@H](C(=O)NCCc1cc(C2CC2)no1)CC(=O)N(C)[C@@H](CC(C)C)C(=O)N(C)CC(=O)N[C@H](Cc1ccccc1)CO3] | 532 |
| 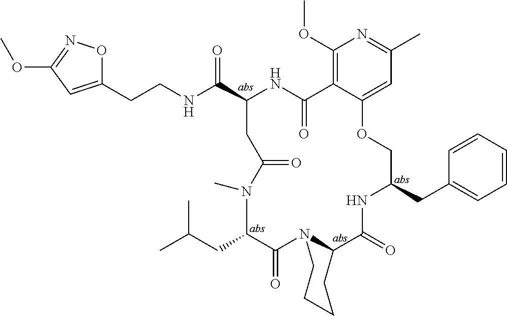 | 533 |
| 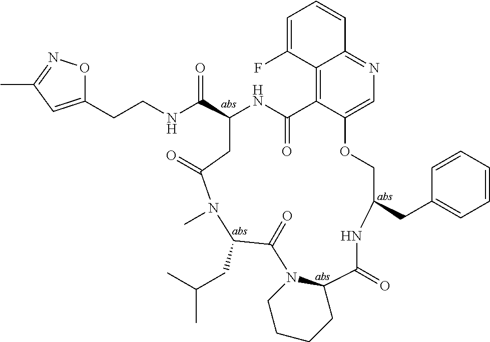 [SMILES: Cc1cc(CCNC(=O)[C@@H]2CC(=O)N(C)[C@@H](CC(C)C)C(=O)N3CCCC[C@@H]3C(=O)N[C@H](Cc3ccccc3)COc3cnc4cccc(F)c4c3C(=O)N2)on1] | 534 |
| 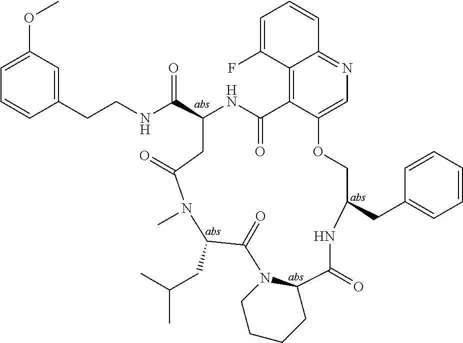 [SMILES: COc1cccc(CCNC(=O)[C@@H]2CC(=O)N(C)[C@@H](CC(C)C)C(=O)N3CCCC[C@@H]3C(=O)N[C@H](Cc3ccccc3)COc3cnc4cccc(F)c4c3C(=O)N2)c1] | 535 |
| 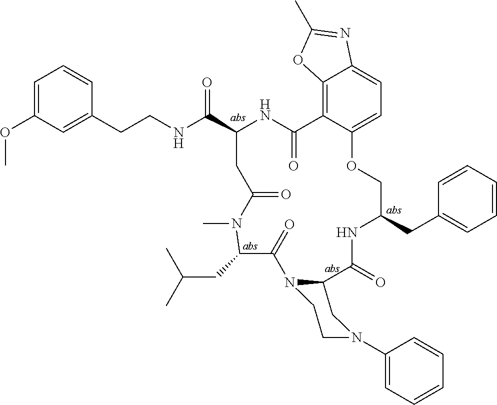 | 536 |
| 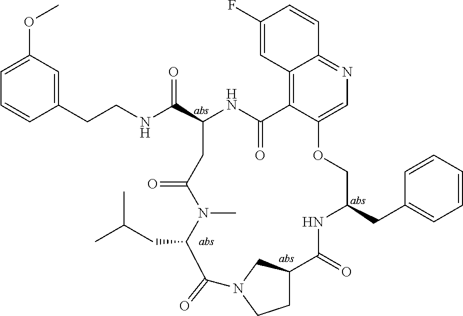 [SMILES: COc1cccc(CCNC(=O)[C@@H]2CC(=O)N(C)[C@@H](CC(C)C)C(=O)N3CC[C@@H](C3)C(=O)N[C@H](Cc3ccccc3)COc3cnc4ccc(F)cc4c3C(=O)N2)c1] | 537 |
| 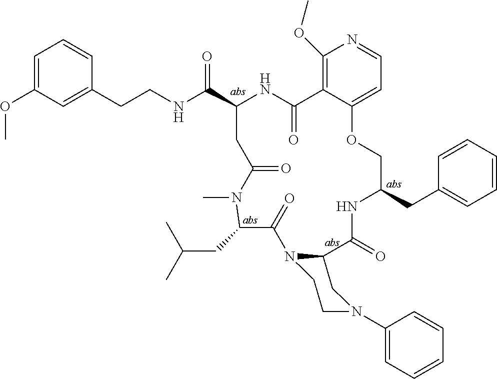 | 538 |
| 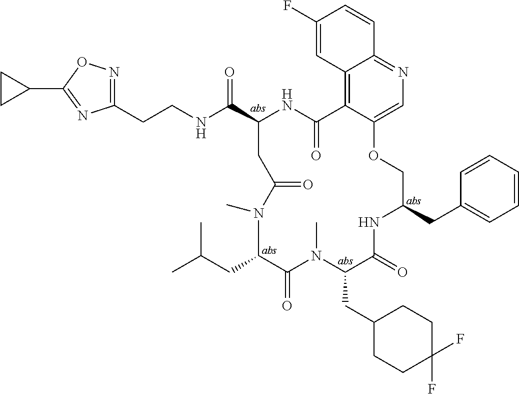 [SMILES: CC(C)C[C@H]1C(=O)N(C)[C@@H](CC2CCC(F)(F)CC2)C(=O)N[C@H](Cc2ccccc2)COc2cnc3ccc(F)cc3c2C(=O)N[C@H](C(=O)NCCc2noc(C3CC3)n2)CC(=O)N1C] | 539 |
| 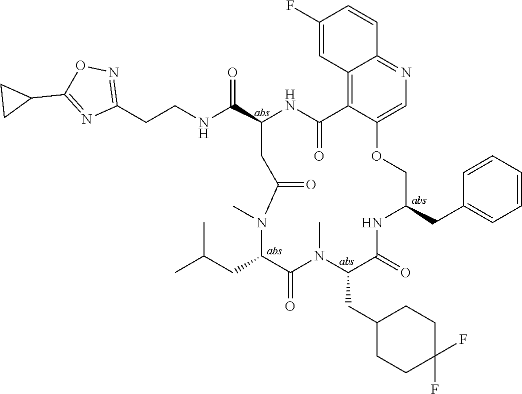 [SMILES: CC(C)C[C@H]1C(=O)N(C)[C@@H](CC2CCC(F)(F)CC2)C(=O)N[C@H](Cc2ccccc2)COc2cnc3ccc(F)cc3c2C(=O)N[C@H](C(=O)NCCc2noc(C3CC3)n2)CC(=O)N1C] | 540 |
| 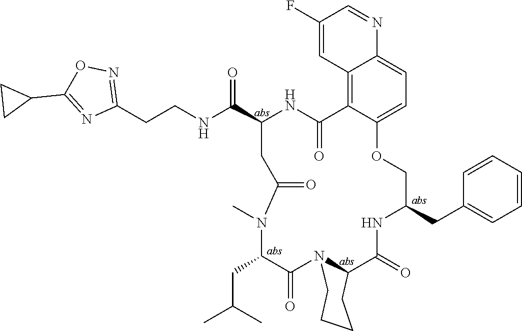 | 541 |
| 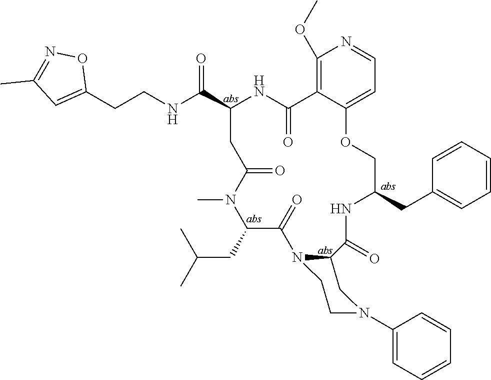 | 542 |
| 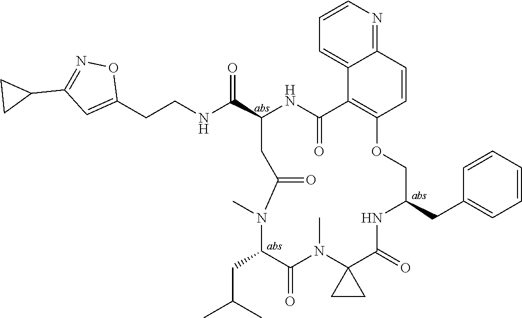 [SMILES: CC(C)C[C@H]1C(=O)N(C)C2(CC2)C(=O)N[C@H](Cc2ccccc2)COc2ccc3ncccc3c2C(=O)N[C@H](C(=O)NCCc2cc(C3CC3)no2)CC(=O)N1C] | 543 |
| 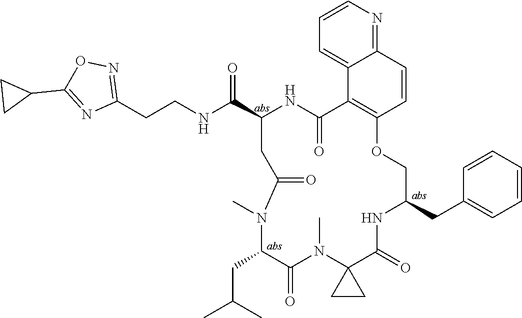 [SMILES: CC(C)C[C@H]1C(=O)N(C)C2(CC2)C(=O)N[C@H](Cc2ccccc2)COc2ccc3ncccc3c2C(=O)N[C@H](C(=O)NCCc2noc(C3CC3)n2)CC(=O)N1C] | 544 |
| 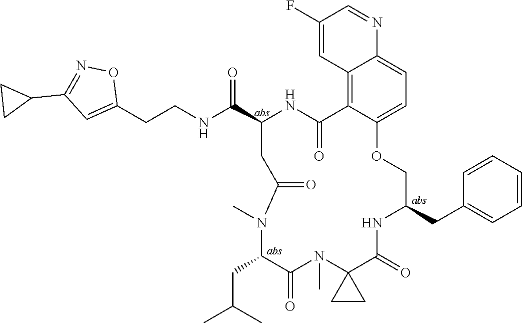 [SMILES: CC(C)C[C@H]1C(=O)N(C)C2(CC2)C(=O)N[C@H](Cc2ccccc2)COc2ccc3ncc(F)cc3c2C(=O)N[C@H](C(=O)NCCc2cc(C3CC3)no2)CC(=O)N1C] | 545 |
| 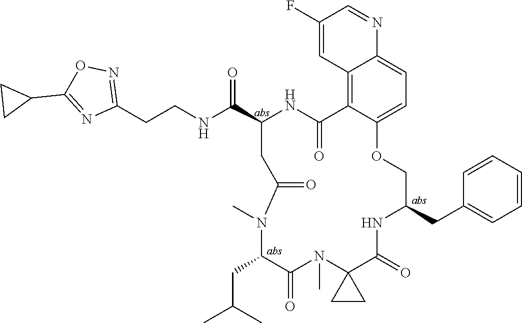 [SMILES: CC(C)C[C@H]1C(=O)N(C)C2(CC2)C(=O)N[C@H](Cc2ccccc2)COc2ccc3ncc(F)cc3c2C(=O)N[C@H](C(=O)NCCc2noc(C3CC3)n2)CC(=O)N1C] | 546 |
| 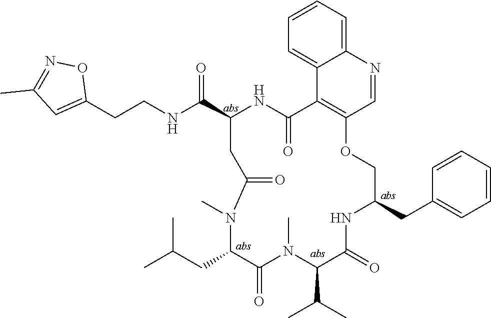 [SMILES: Cc1cc(CCNC(=O)[C@@H]2CC(=O)N(C)[C@@H](CC(C)C)C(=O)N(C)[C@H](C(C)C)C(=O)N[C@H](Cc3ccccc3)COc3cnc4ccccc4c3C(=O)N2)on1] | 547 |
| 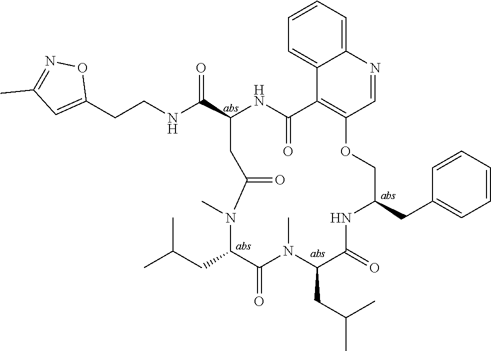 [SMILES: Cc1cc(CCNC(=O)[C@@H]2CC(=O)N(C)[C@@H](CC(C)C)C(=O)N(C)[C@H](CC(C)C)C(=O)N[C@H](Cc3ccccc3)COc3cnc4ccccc4c3C(=O)N2)on1] | 548 |
| 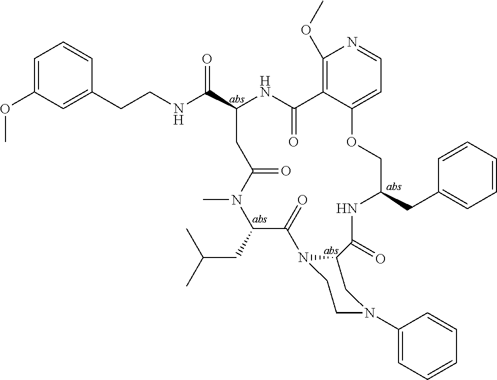 | 549 |
| 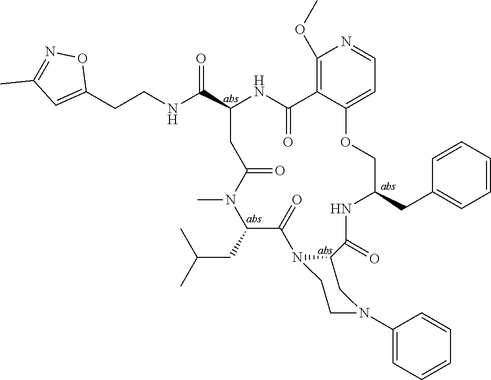 | 550 |
| 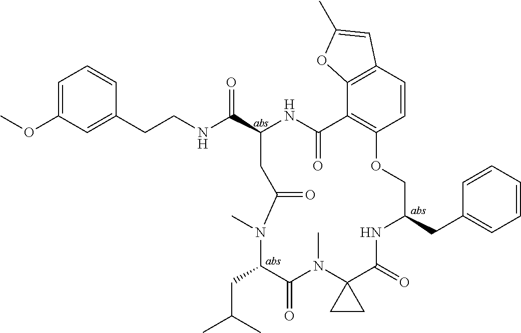 [SMILES: COc1cccc(CCNC(=O)[C@@H]2CC(=O)N(C)[C@@H](CC(C)C)C(=O)N(C)C3(CC3)C(=O)N[C@H](Cc3ccccc3)COc3ccc4cc(C)oc4c3C(=O)N2)c1] | 551 |
| 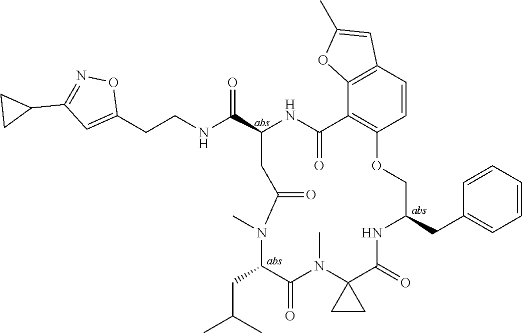 [SMILES: Cc1cc2ccc3c(c2o1)C(=O)N[C@H](C(=O)NCCc1cc(C2CC2)no1)CC(=O)N(C)[C@@H](CC(C)C)C(=O)N(C)C1(CC1)C(=O)N[C@H](Cc1ccccc1)CO3] | 552 |
| 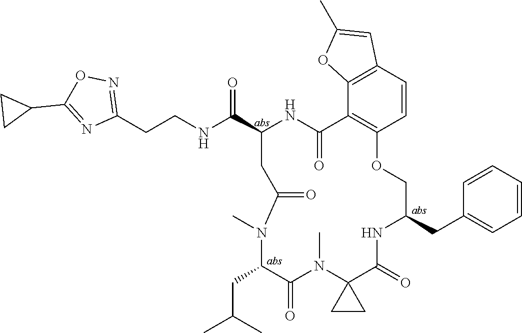 [SMILES: Cc1cc2ccc3c(c2o1)C(=O)N[C@H](C(=O)NCCc1noc(C2CC2)n1)CC(=O)N(C)[C@@H](CC(C)C)C(=O)N(C)C1(CC1)C(=O)N[C@H](Cc1ccccc1)CO3] | 553 |
| 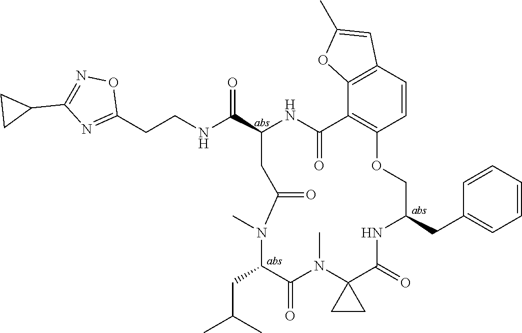 [SMILES: Cc1cc2ccc3c(c2o1)C(=O)N[C@H](C(=O)NCCc1nc(C2CC2)no1)CC(=O)N(C)[C@@H](CC(C)C)C(=O)N(C)C1(CC1)C(=O)N[C@H](Cc1ccccc1)CO3] | 554 |
| 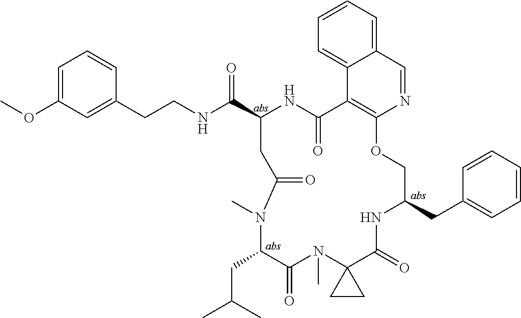 [SMILES: COc1cccc(CCNC(=O)[C@@H]2CC(=O)N(C)[C@@H](CC(C)C)C(=O)N(C)C3(CC3)C(=O)N[C@H](Cc3ccccc3)COc3ncc4ccccc4c3C(=O)N2)c1] | 555 |
| 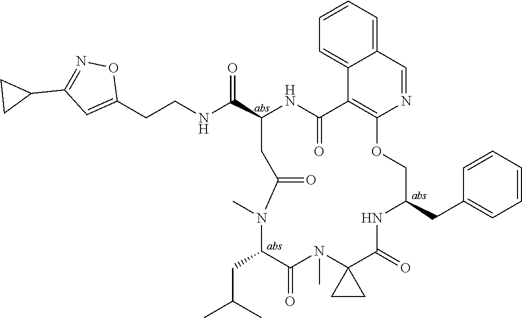 [SMILES: CC(C)C[C@H]1C(=O)N(C)C2(CC2)C(=O)N[C@H](Cc2ccccc2)COc2ncc3ccccc3c2C(=O)N[C@H](C(=O)NCCc2cc(C3CC3)no2)CC(=O)N1C] | 556 |
| 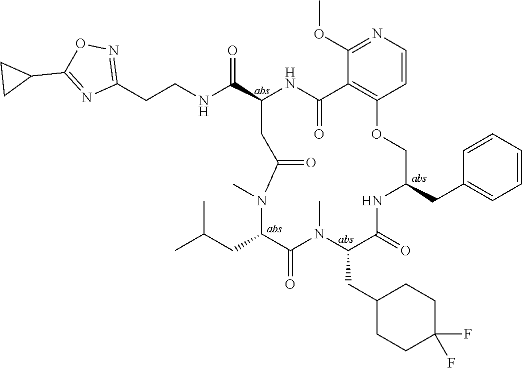 [SMILES: COc1nccc2c1C(=O)N[C@H](C(=O)NCCc1noc(C3CC3)n1)CC(=O)N(C)[C@@H](CC(C)C)C(=O)N(C)[C@@H](CC1CCC(F)(F)CC1)C(=O)N[C@H](Cc1ccccc1)CO2] | 557 |
| 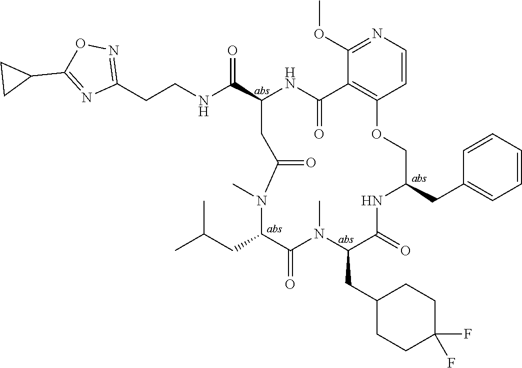 [SMILES: COc1nccc2c1C(=O)N[C@H](C(=O)NCCc1noc(C3CC3)n1)CC(=O)N(C)[C@@H](CC(C)C)C(=O)N(C)[C@H](CC1CCC(F)(F)CC1)C(=O)N[C@H](Cc1ccccc1)CO2] | 558 |
| 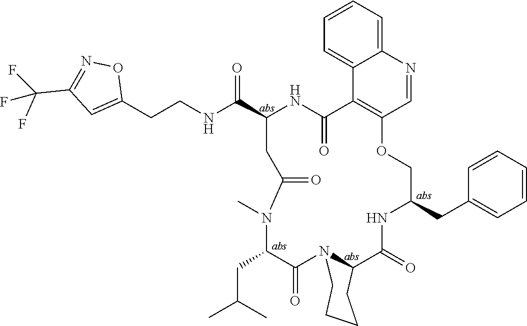 | 559 |
| 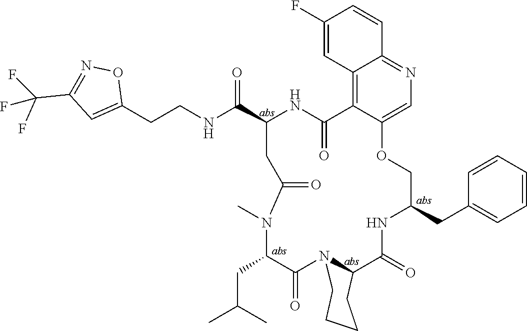 | 560 |
| 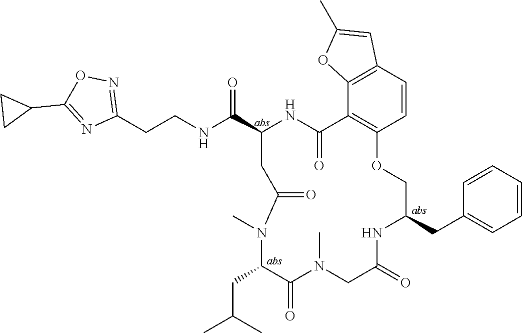 [SMILES: Cc1cc2ccc3c(c2o1)C(=O)N[C@H](C(=O)NCCc1noc(C2CC2)n1)CC(=O)N(C)[C@@H](CC(C)C)C(=O)N(C)CC(=O)N[C@H](Cc1ccccc1)CO3] | 561 |
| 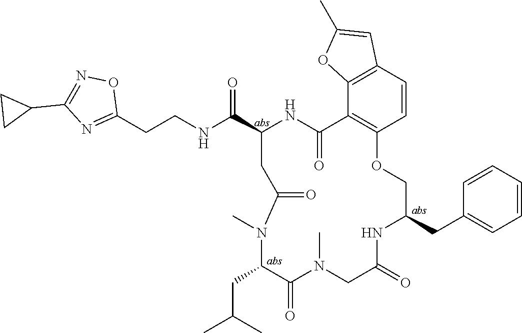 [SMILES: Cc1cc2ccc3c(c2o1)C(=O)N[C@H](C(=O)NCCc1nc(C2CC2)no1)CC(=O)N(C)[C@@H](CC(C)C)C(=O)N(C)CC(=O)N[C@H](Cc1ccccc1)CO3] | 562 |
| 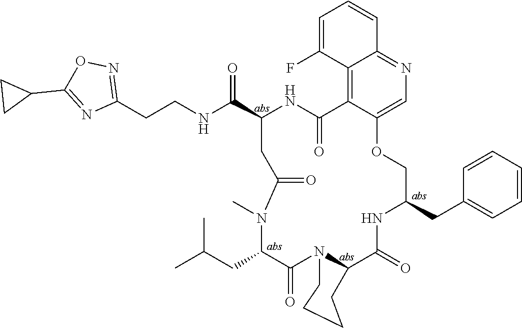 | 563 |
| 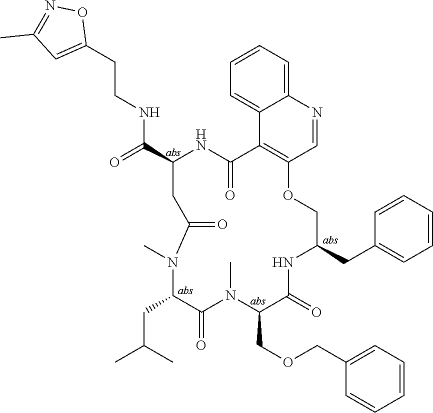 [SMILES: Cc1cc(CCNC(=O)[C@@H]2CC(=O)N(C)[C@@H](CC(C)C)C(=O)N(C)[C@H](COCc3ccccc3)C(=O)N[C@H](Cc3ccccc3)COc3cnc4ccccc4c3C(=O)N2)on1] | 564 |
| 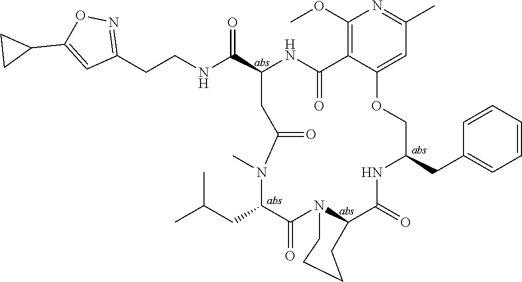 | 565 |
| 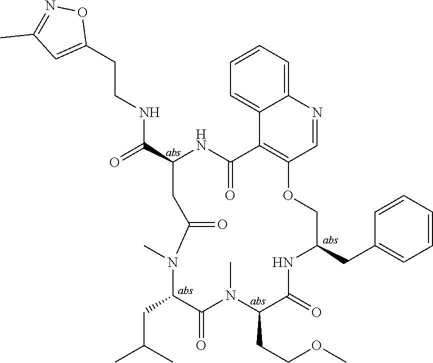 [SMILES: COCC[C@@H]1C(=O)N[C@H](Cc2ccccc2)COc2cnc3ccccc3c2C(=O)N[C@H](C(=O)NCCc2cc(C)no2)CC(=O)N(C)[C@@H](CC(C)C)C(=O)N1C] | 566 |
| 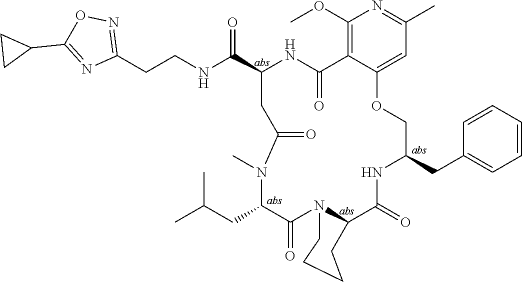 | 567 |
| 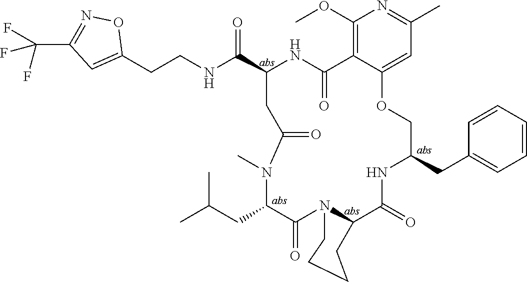 | 568 |
| 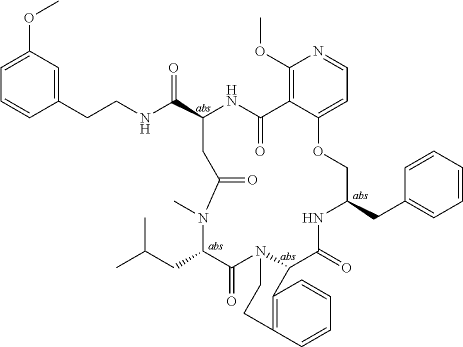 | 569 |
| 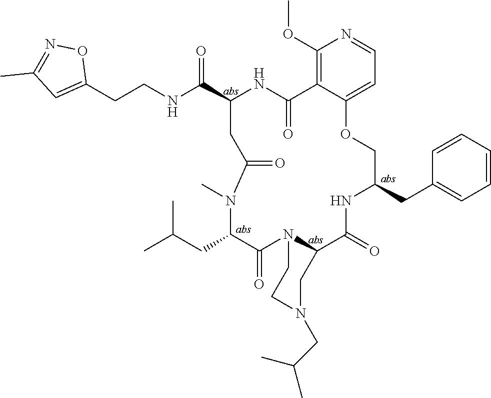 | 570 |
| 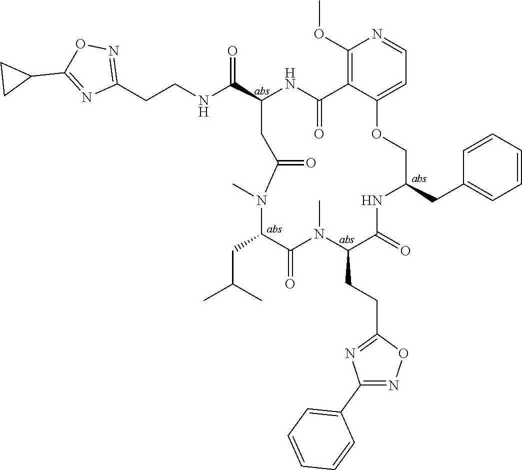 [SMILES: COc1nccc2c1C(=O)N[C@H](C(=O)NCCc1noc(C3CC3)n1)CC(=O)N(C)[C@@H](CC(C)C)C(=O)N(C)[C@H](CCc1nc(-c3ccccc3)no1)C(=O)N[C@H](Cc1ccccc1)CO2] | 571 |
| 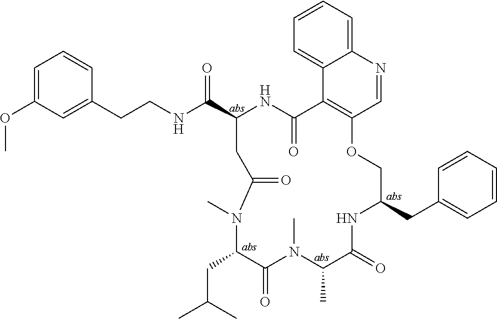 [SMILES: COc1cccc(CCNC(=O)[C@@H]2CC(=O)N(C)[C@@H](CC(C)C)C(=O)N(C)[C@@H](C)C(=O)N[C@H](Cc3ccccc3)COc3cnc4ccccc4c3C(=O)N2)c1] | 572 |
| 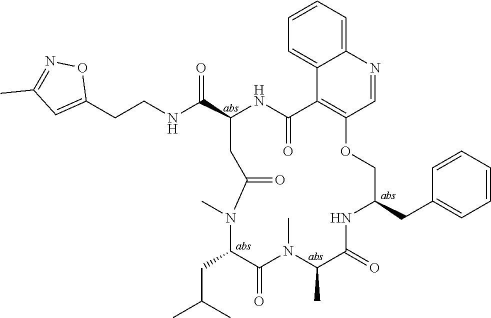 [SMILES: Cc1cc(CCNC(=O)[C@@H]2CC(=O)N(C)[C@@H](CC(C)C)C(=O)N(C)[C@H](C)C(=O)N[C@H](Cc3ccccc3)COc3cnc4ccccc4c3C(=O)N2)on1] | 573 |
| 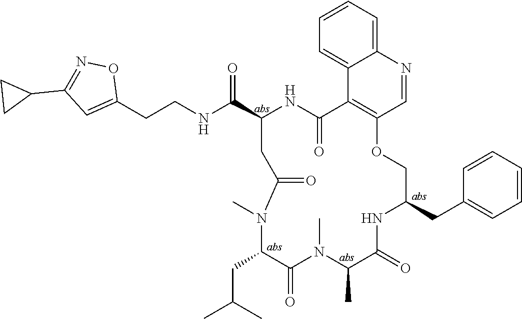 [SMILES: CC(C)C[C@H]1C(=O)N(C)[C@H](C)C(=O)N[C@H](Cc2ccccc2)COc2cnc3ccccc3c2C(=O)N[C@H](C(=O)NCCc2cc(C3CC3)no2)CC(=O)N1C] | 574 |
| 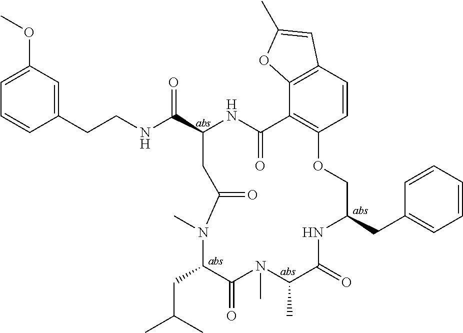 [SMILES: COc1cccc(CCNC(=O)[C@@H]2CC(=O)N(C)[C@@H](CC(C)C)C(=O)N(C)[C@@H](C)C(=O)N[C@H](Cc3ccccc3)COc3ccc4cc(C)oc4c3C(=O)N2)c1] | 575 |
| 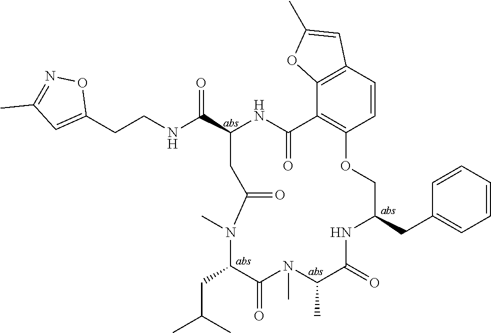 [SMILES: Cc1cc(CCNC(=O)[C@@H]2CC(=O)N(C)[C@@H](CC(C)C)C(=O)N(C)[C@@H](C)C(=O)N[C@H](Cc3ccccc3)COc3ccc4cc(C)oc4c3C(=O)N2)on1] | 576 |
| 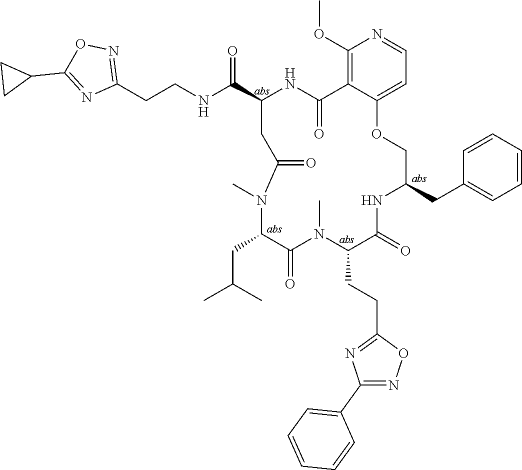 [SMILES: COc1nccc2c1C(=O)N[C@H](C(=O)NCCc1noc(C3CC3)n1)CC(=O)N(C)[C@@H](CC(C)C)C(=O)N(C)[C@@H](CCc1nc(-c3ccccc3)no1)C(=O)N[C@H](Cc1ccccc1)CO2] | 577 |
| 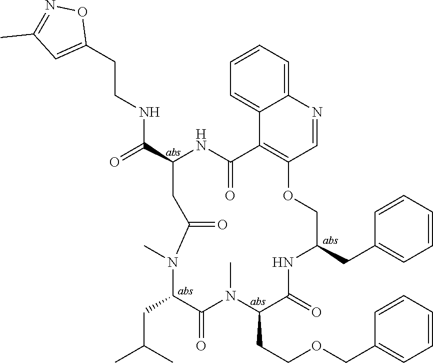 [SMILES: Cc1cc(CCNC(=O)[C@@H]2CC(=O)N(C)[C@@H](CC(C)C)C(=O)N(C)[C@H](CCOCc3ccccc3)C(=O)N[C@H](Cc3ccccc3)COc3cnc4ccccc4c3C(=O)N2)on1] | 578 |
| 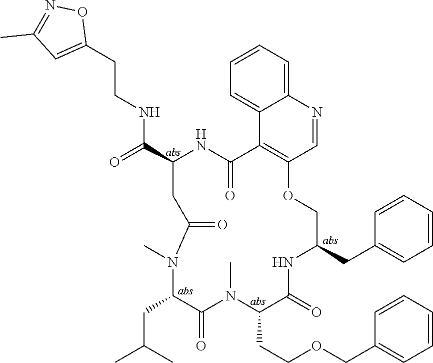 [SMILES: Cc1cc(CCNC(=O)[C@@H]2CC(=O)N(C)[C@@H](CC(C)C)C(=O)N(C)[C@@H](CCOCc3ccccc3)C(=O)N[C@H](Cc3ccccc3)COc3cnc4ccccc4c3C(=O)N2)on1] | 579 |
| 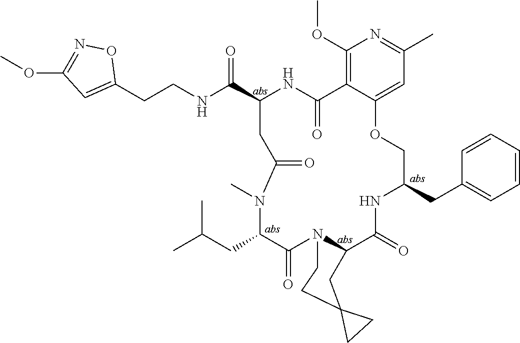 | 580 |
| 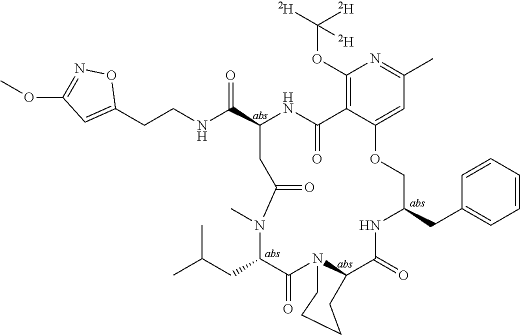 | 581 |
| 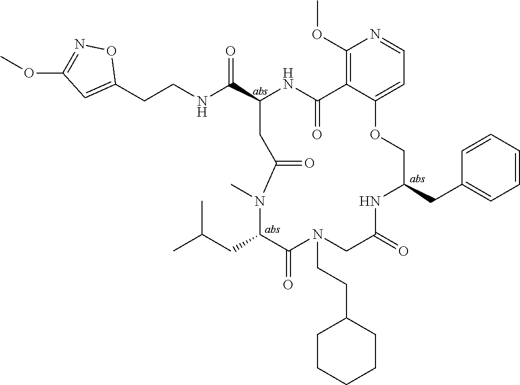 [SMILES: COc1cc(CCNC(=O)[C@@H]2CC(=O)N(C)[C@@H](CC(C)C)C(=O)N(CCC3CCCCC3)CC(=O)N[C@H](Cc3ccccc3)COc3ccnc(OC)c3C(=O)N2)on1] | 582 |
| 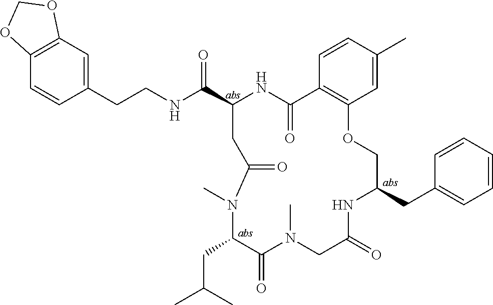 [SMILES: Cc1ccc2c(c1)OC[C@@H](Cc1ccccc1)NC(=O)CN(C)C(=O)[C@H](CC(C)C)N(C)C(=O)C[C@@H](C(=O)NCCc1ccc3c(c1)OCO3)NC2=O] | 583 |
| 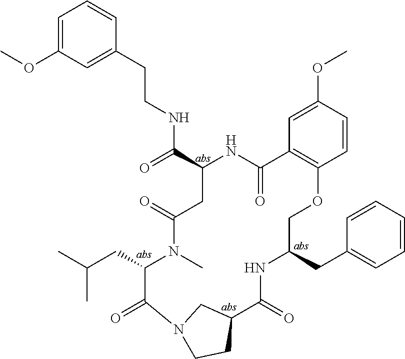 [SMILES: COc1cccc(CCNC(=O)[C@@H]2CC(=O)N(C)[C@@H](CC(C)C)C(=O)N3CC[C@@H](C3)C(=O)N[C@H](Cc3ccccc3)COc3ccc(OC)cc3C(=O)N2)c1] | 584 |
| 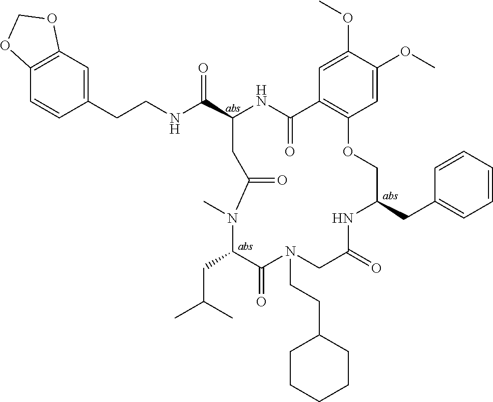 [SMILES: COc1cc2c(cc1OC)C(=O)N[C@H](C(=O)NCCc1ccc3c(c1)OCO3)CC(=O)N(C)[C@@H](CC(C)C)C(=O)N(CCC1CCCCC1)CC(=O)N[C@H](Cc1ccccc1)CO2] | 585 |
| 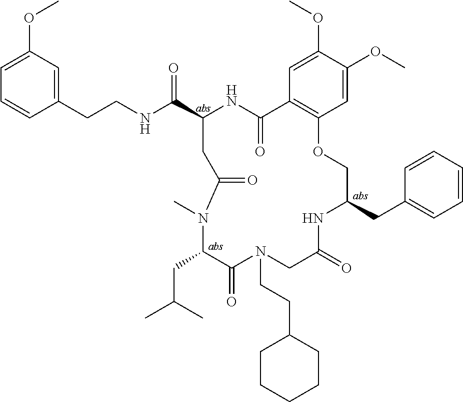 [SMILES: COc1cccc(CCNC(=O)[C@@H]2CC(=O)N(C)[C@@H](CC(C)C)C(=O)N(CCC3CCCCC3)CC(=O)N[C@H](Cc3ccccc3)COc3cc(OC)c(OC)cc3C(=O)N2)c1] | 586 |
| 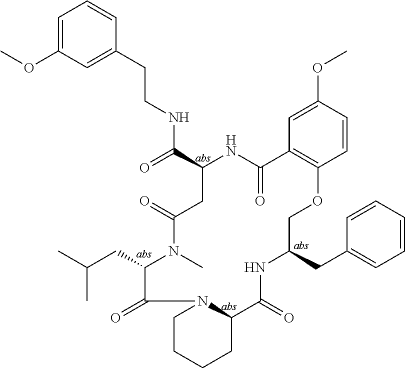 [SMILES: COc1cccc(CCNC(=O)[C@@H]2CC(=O)N(C)[C@@H](CC(C)C)C(=O)N3CCCC[C@@H]3C(=O)N[C@H](Cc3ccccc3)COc3ccc(OC)cc3C(=O)N2)c1] | 587 |
| 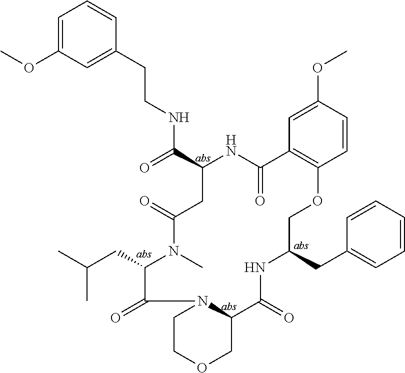 [SMILES: COc1cccc(CCNC(=O)[C@@H]2CC(=O)N(C)[C@@H](CC(C)C)C(=O)N3CCOC[C@@H]3C(=O)N[C@H](Cc3ccccc3)COc3ccc(OC)cc3C(=O)N2)c1] | 588 |
| 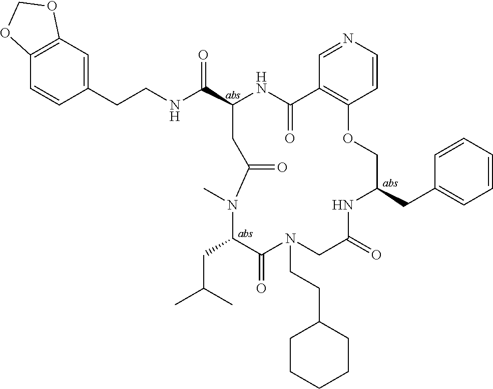 [SMILES: CC(C)C[C@H]1C(=O)N(CCC2CCCCC2)CC(=O)N[C@H](Cc2ccccc2)COc2ccncc2C(=O)N[C@H](C(=O)NCCc2ccc3c(c2)OCO3)CC(=O)N1C] | 589 |
| 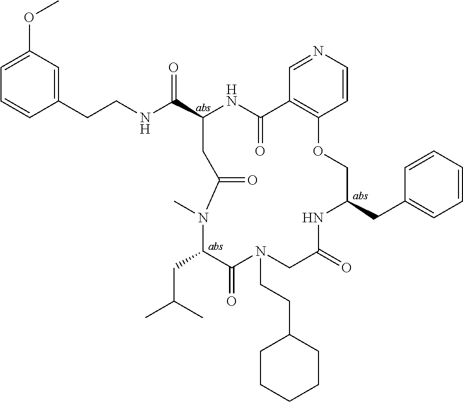 [SMILES: COc1cccc(CCNC(=O)[C@@H]2CC(=O)N(C)[C@@H](CC(C)C)C(=O)N(CCC3CCCCC3)CC(=O)N[C@H](Cc3ccccc3)COc3ccncc3C(=O)N2)c1] | 590 |
| 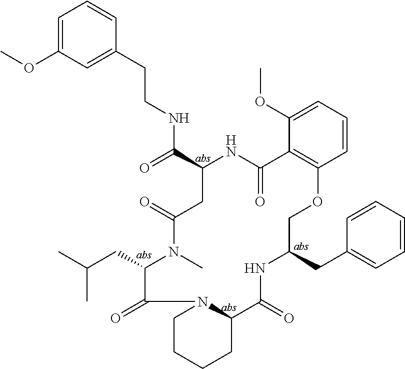 [SMILES: COc1cccc(CCNC(=O)[C@@H]2CC(=O)N(C)[C@@H](CC(C)C)C(=O)N3CCCC[C@@H]3C(=O)N[C@H](Cc3ccccc3)COc3cccc(OC)c3C(=O)N2)c1] | 591 |
| 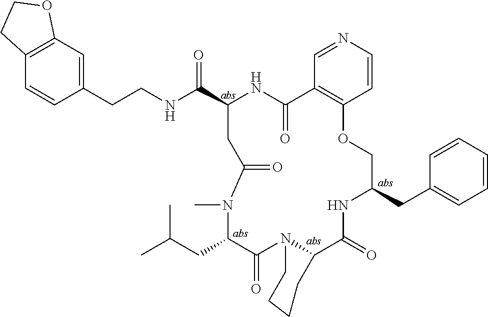 | 592 |
| 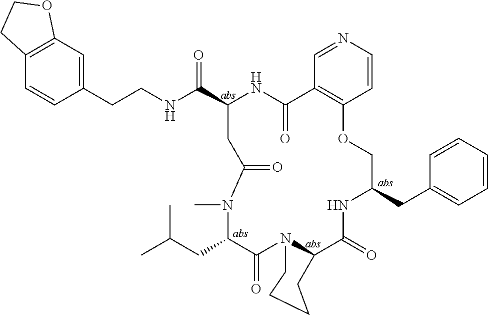 | 593 |
| 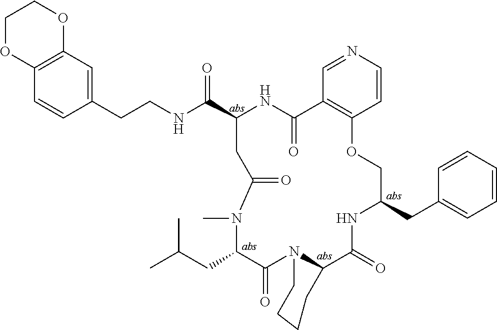 | 594 |
| 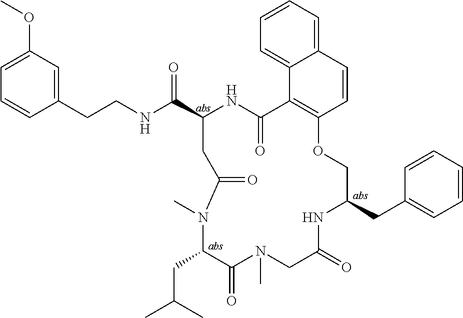 [SMILES: COc1cccc(CCNC(=O)[C@@H]2CC(=O)N(C)[C@@H](CC(C)C)C(=O)N(C)CC(=O)N[C@H](Cc3ccccc3)COc3ccc4ccccc4c3C(=O)N2)c1] | 595 |
| 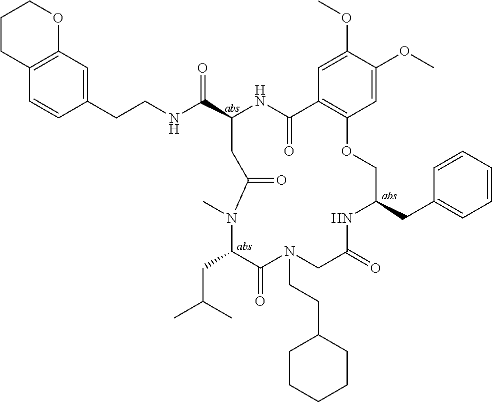 [SMILES: COc1cc2c(cc1OC)C(=O)N[C@H](C(=O)NCCc1ccc3c(c1)OCCC3)CC(=O)N(C)[C@@H](CC(C)C)C(=O)N(CCC1CCCCC1)CC(=O)N[C@H](Cc1ccccc1)CO2] | 596 |
| 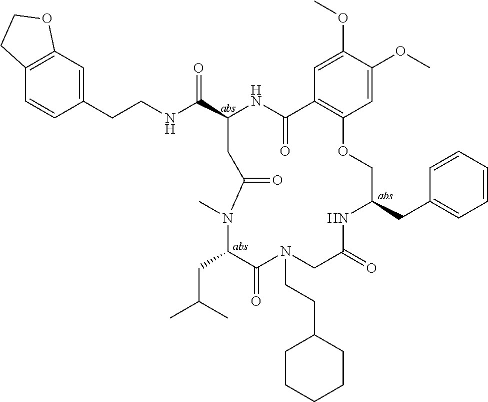 [SMILES: COc1cc2c(cc1OC)C(=O)N[C@H](C(=O)NCCc1ccc3c(c1)OCC3)CC(=O)N(C)[C@@H](CC(C)C)C(=O)N(CCC1CCCCC1)CC(=O)N[C@H](Cc1ccccc1)CO2] | 597 |
| 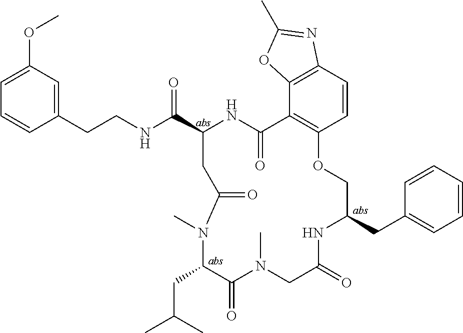 [SMILES: COc1cccc(CCNC(=O)[C@@H]2CC(=O)N(C)[C@@H](CC(C)C)C(=O)N(C)CC(=O)N[C@H](Cc3ccccc3)COc3ccc4nc(C)oc4c3C(=O)N2)c1] | 598 |
| 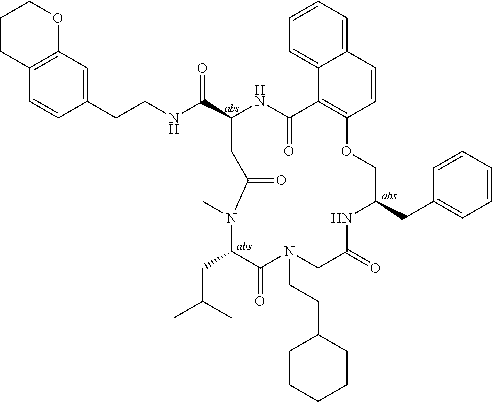 [SMILES: CC(C)C[C@H]1C(=O)N(CCC2CCCCC2)CC(=O)N[C@H](Cc2ccccc2)COc2ccc3ccccc3c2C(=O)N[C@H](C(=O)NCCc2ccc3c(c2)OCCC3)CC(=O)N1C] | 599 |
| 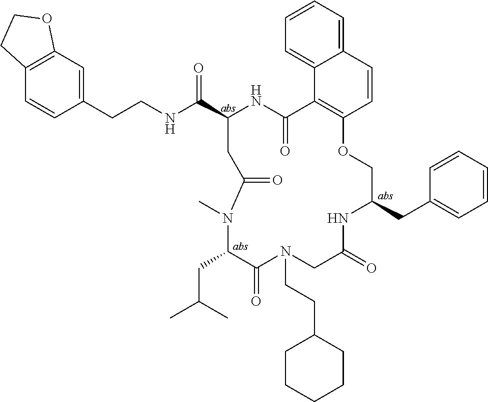 [SMILES: CC(C)C[C@H]1C(=O)N(CCC2CCCCC2)CC(=O)N[C@H](Cc2ccccc2)COc2ccc3ccccc3c2C(=O)N[C@H](C(=O)NCCc2ccc3c(c2)OCC3)CC(=O)N1C] | 600 |
| 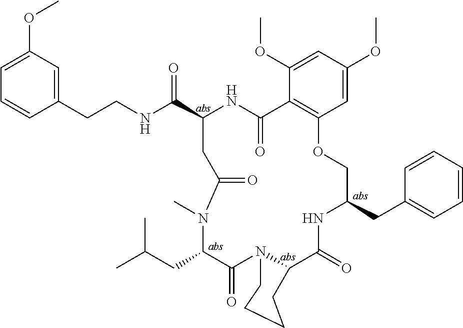 | 601 |
| 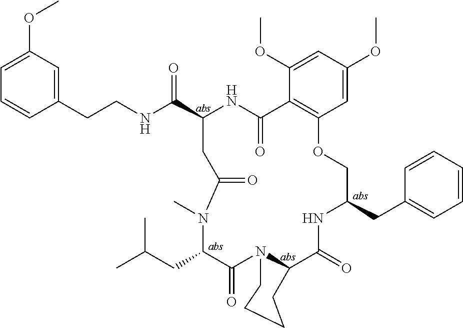 | 602 |
| 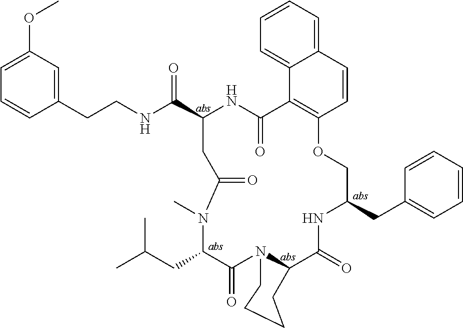 | 603 |
| 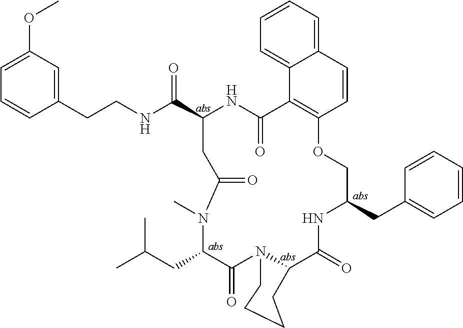 | 604 |
| 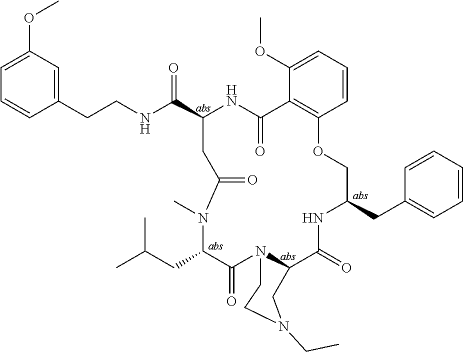 | 605 |
| 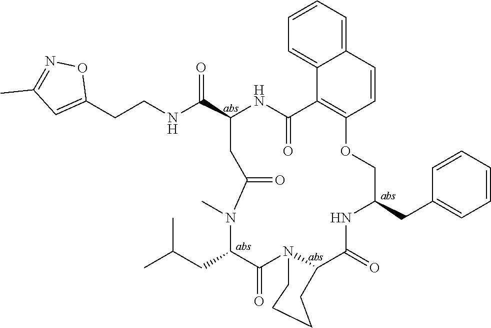 | 606 |
| 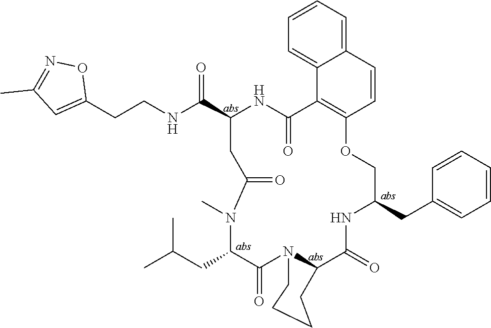 | 607 |
| 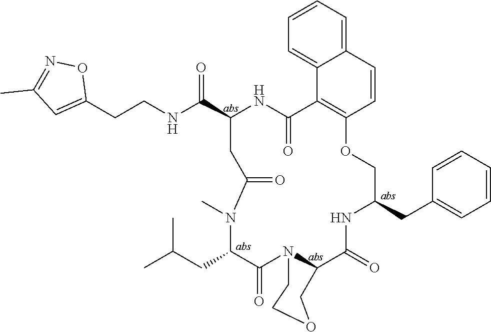 | 608 |
| 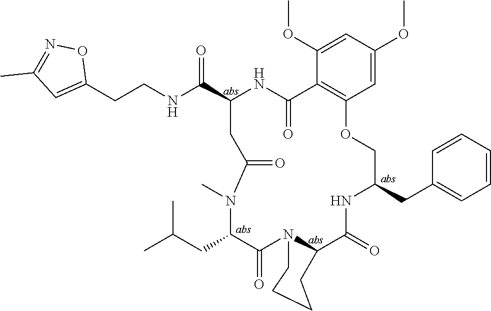 | 609 |
| 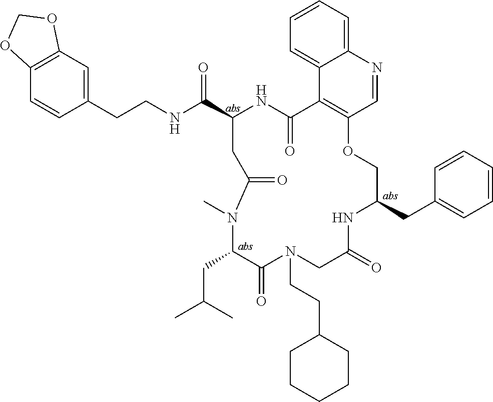 [SMILES: CC(C)C[C@H]1C(=O)N(CCC2CCCCC2)CC(=O)N[C@H](Cc2ccccc2)COc2cnc3ccccc3c2C(=O)N[C@H](C(=O)NCCc2ccc3c(c2)OCO3)CC(=O)N1C] | 610 |
| 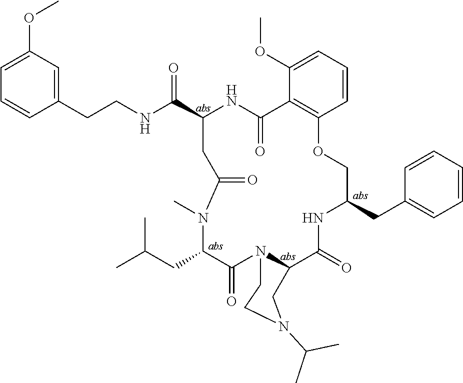 | 611 |
| 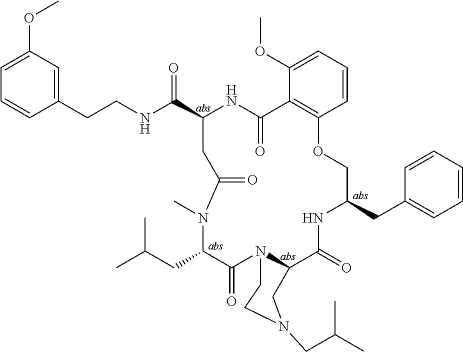 | 612 |
| 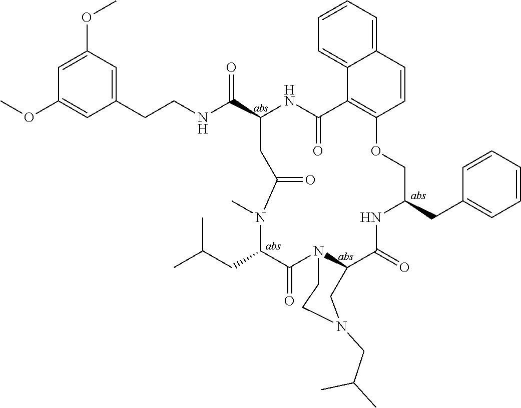 | 613 |
| 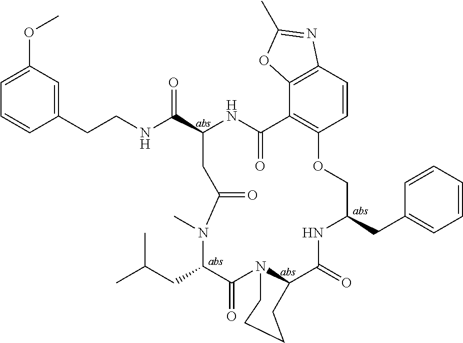 | 614 |
| 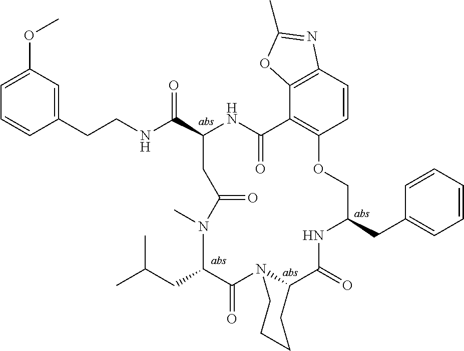 | 615 |
| 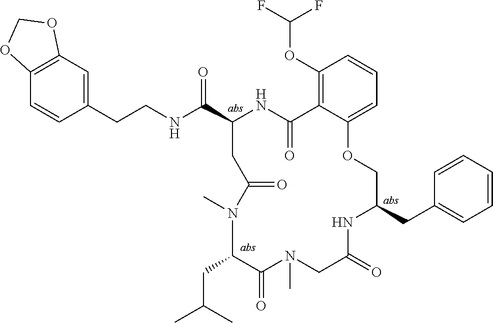 [SMILES: CC(C)C[C@H]1C(=O)N(C)CC(=O)N[C@H](Cc2ccccc2)COc2cccc(OC(F)F)c2C(=O)N[C@H](C(=O)NCCc2ccc3c(c2)OCO3)CC(=O)N1C] | 616 |
| 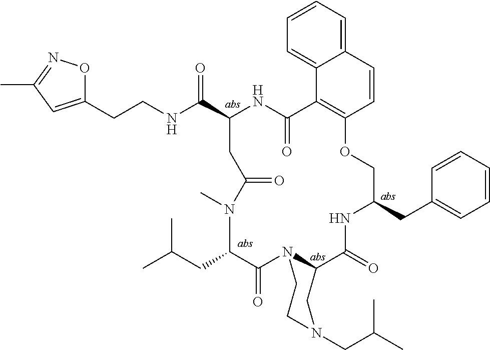 | 617 |
| 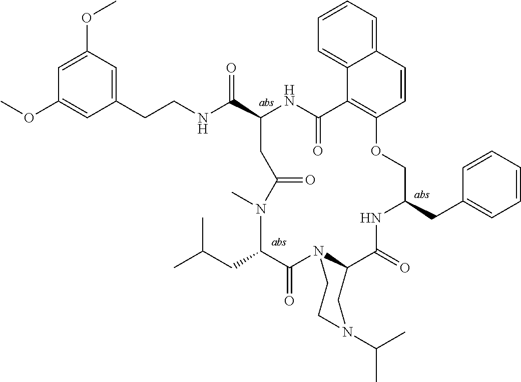 | 618 |
| 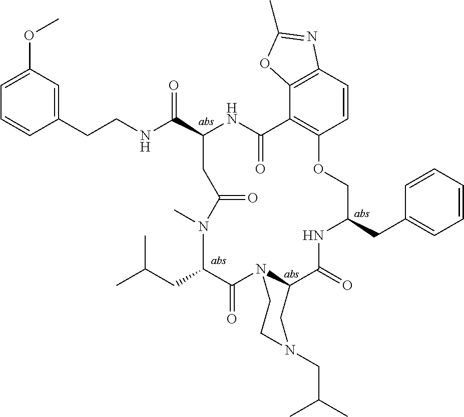 | 619 |
| 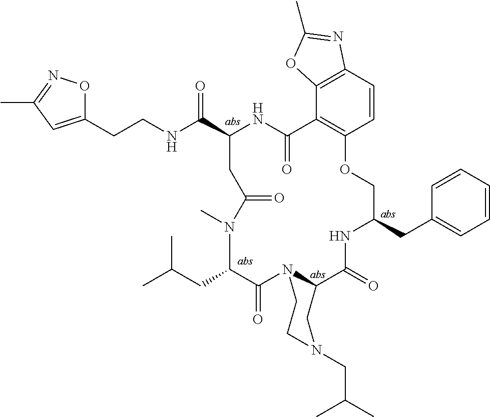 | 620 |
| 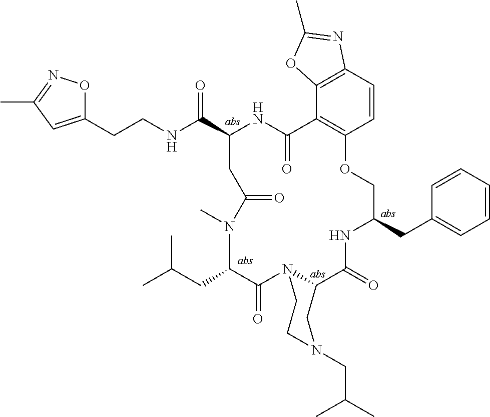 | 621 |
| 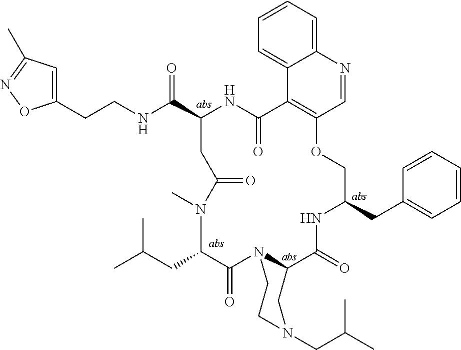 | 622 |
| 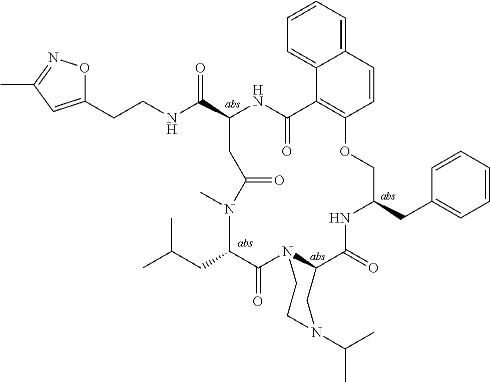 | 623 |
| 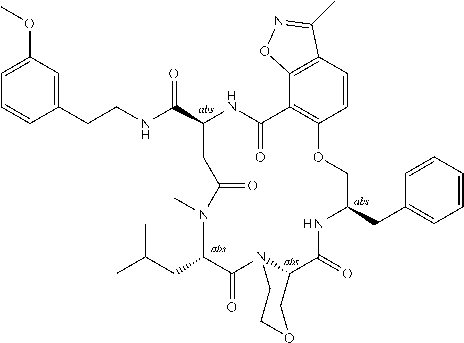 | 624 |
| 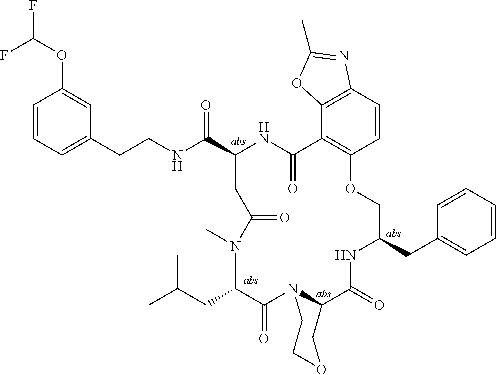 | 625 |
| 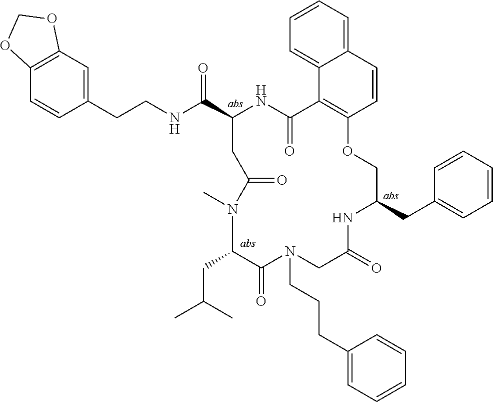 [SMILES: CC(C)C[C@H]1C(=O)N(CCCc2ccccc2)CC(=O)N[C@H](Cc2ccccc2)COc2ccc3ccccc3c2C(=O)N[C@H](C(=O)NCCc2ccc3c(c2)OCO3)CC(=O)N1C] | 626 |
| 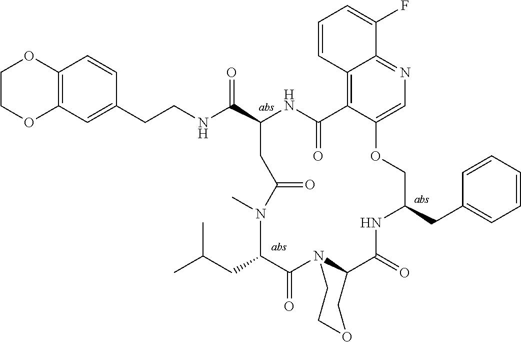 | 627 |
| 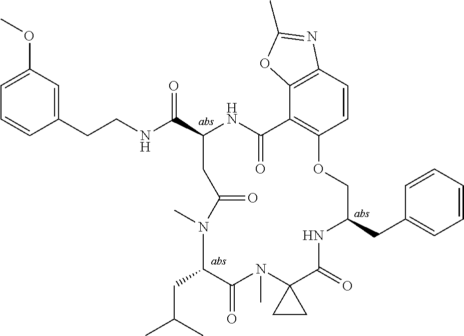 [SMILES: COc1cccc(CCNC(=O)[C@@H]2CC(=O)N(C)[C@@H](CC(C)C)C(=O)N(C)C3(CC3)C(=O)N[C@H](Cc3ccccc3)COc3ccc4nc(C)oc4c3C(=O)N2)c1] | 628 |
| 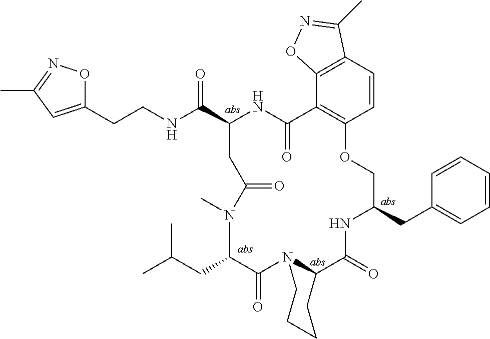 | 629 |
| 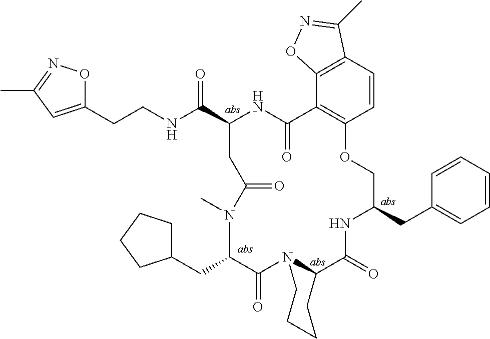 | 630 |
| 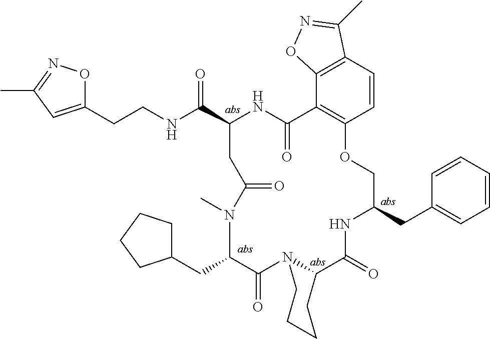 | 631 |
| 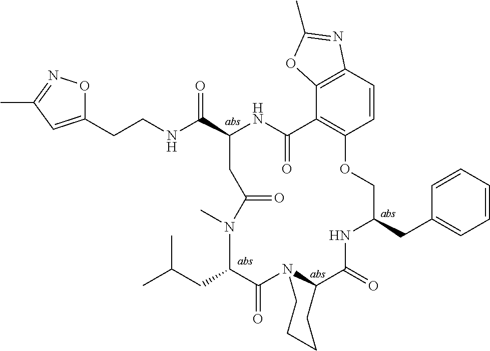 | 632 |
| 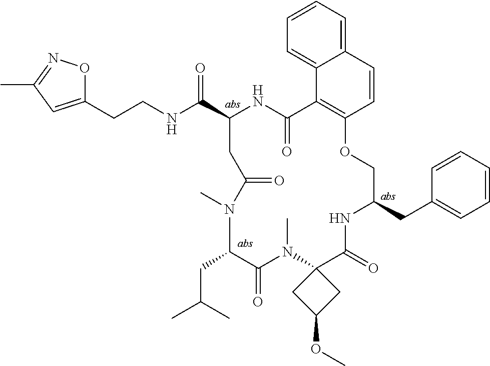 [SMILES: CO[C@H]1C[C@]2(C1)C(=O)N[C@H](Cc1ccccc1)COc1ccc3ccccc3c1C(=O)N[C@H](C(=O)NCCc1cc(C)no1)CC(=O)N(C)[C@@H](CC(C)C)C(=O)N2C] | 633 |
| 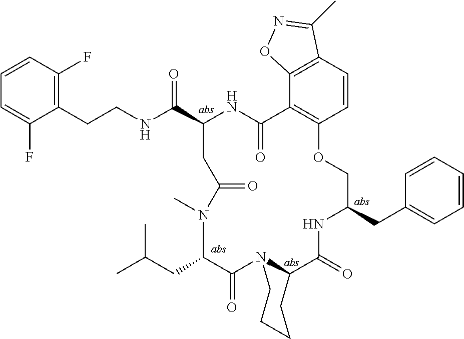 | 634 |
| 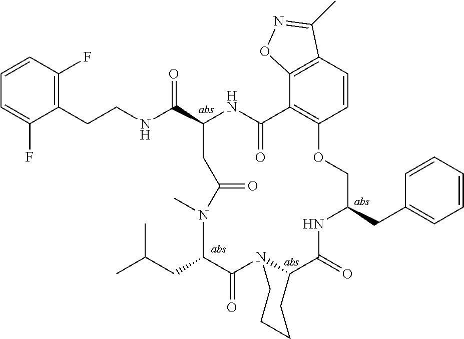 | 635 |
| 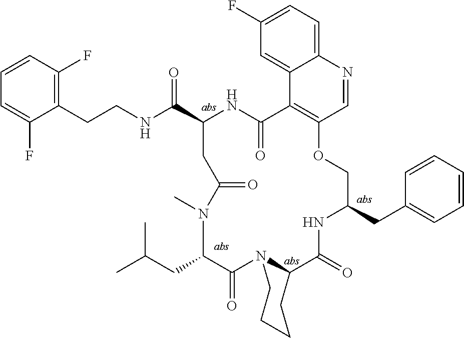 | 636 |
| 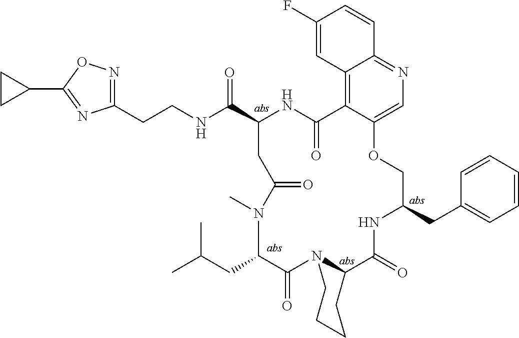 | 637 |
| 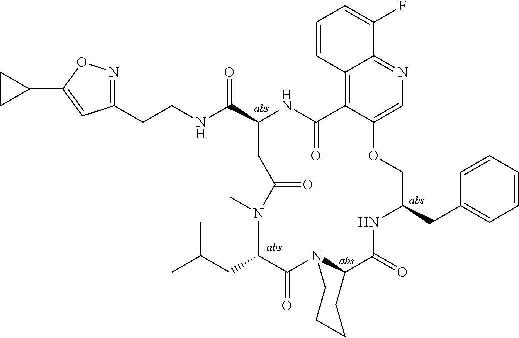 | 638 |
| 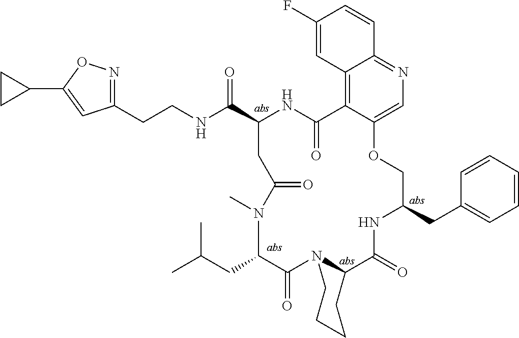 | 639 |
| 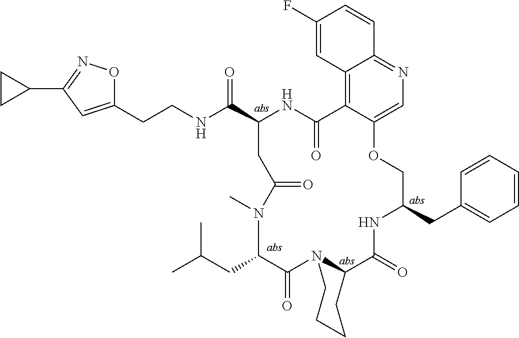 | 640 |
| 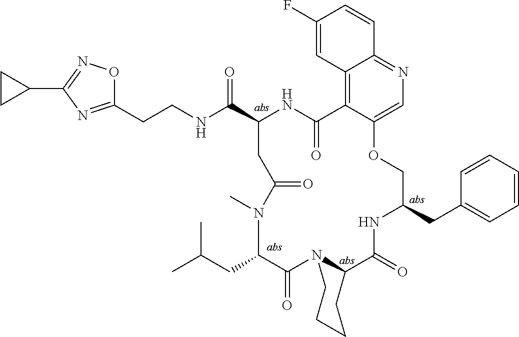 | 641 |
| 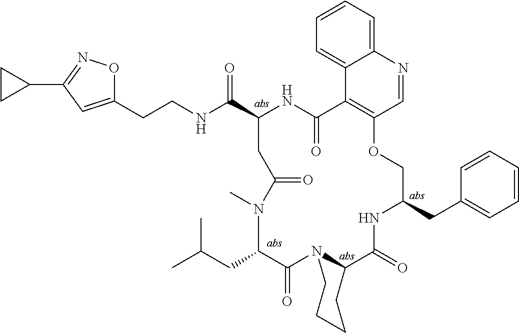 | 642 |
| 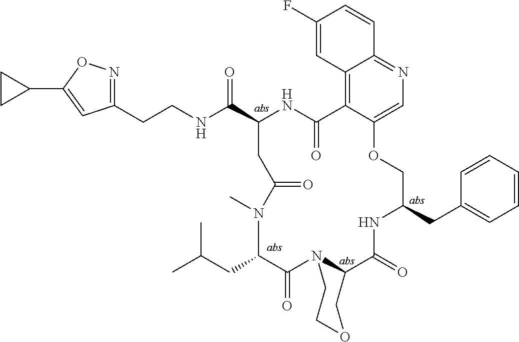 | 643 |
| 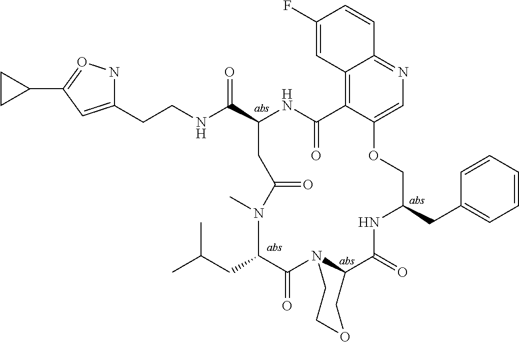 | 644 |
| 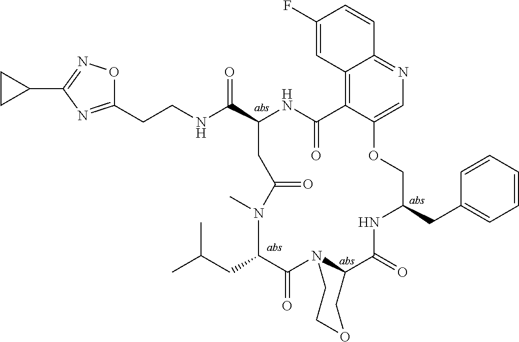 | 645 |
| 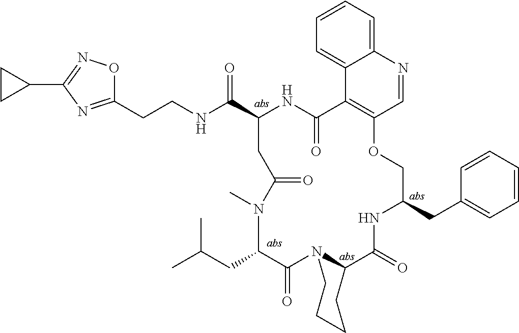 | 646 |
| 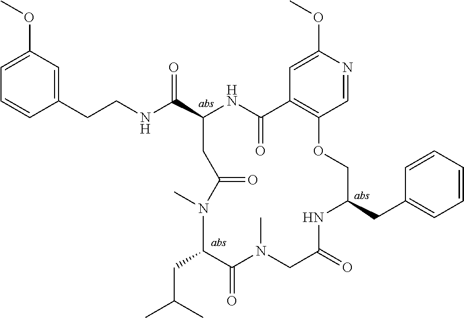 [SMILES: COc1cccc(CCNC(=O)[C@@H]2CC(=O)N(C)[C@@H](CC(C)C)C(=O)N(C)CC(=O)N[C@H](Cc3ccccc3)COc3cnc(OC)cc3C(=O)N2)c1] | 647 |
| 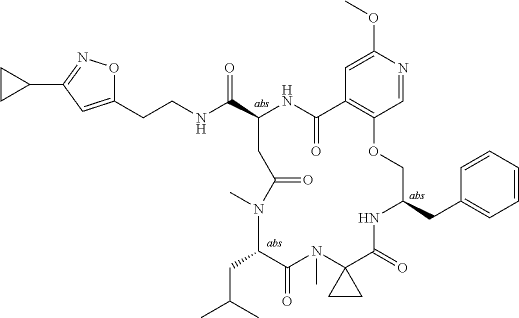 [SMILES: COc1cc2c(cn1)OC[C@@H](Cc1ccccc1)NC(=O)C1(CC1)N(C)C(=O)[C@H](CC(C)C)N(C)C(=O)C[C@@H](C(=O)NCCc1cc(C3CC3)no1)NC2=O] | 648 |
| 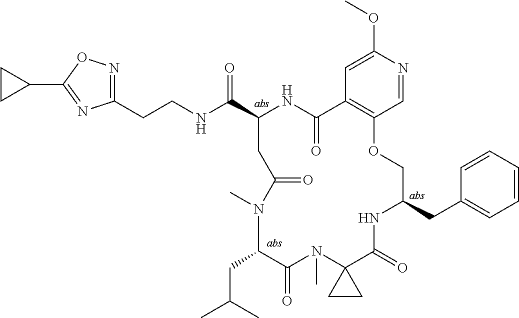 [SMILES: COc1cc2c(cn1)OC[C@@H](Cc1ccccc1)NC(=O)C1(CC1)N(C)C(=O)[C@H](CC(C)C)N(C)C(=O)C[C@@H](C(=O)NCCc1noc(C3CC3)n1)NC2=O] | 649 |
| 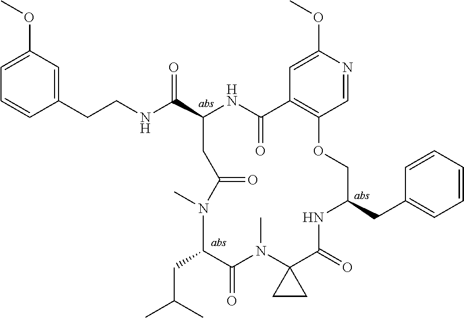 [SMILES: COc1cccc(CCNC(=O)[C@@H]2CC(=O)N(C)[C@@H](CC(C)C)C(=O)N(C)C3(CC3)C(=O)N[C@H](Cc3ccccc3)COc3cnc(OC)cc3C(=O)N2)c1] | 650 |
| 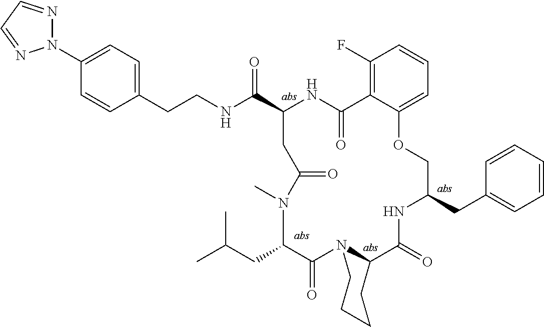 | 651 |
| 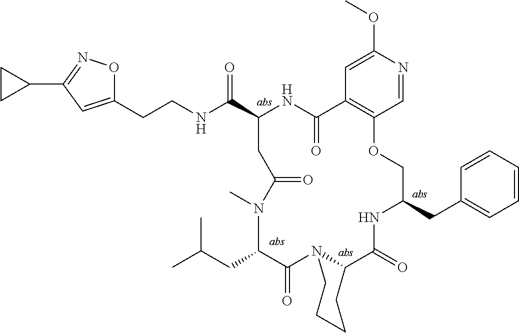 | 652 |
| 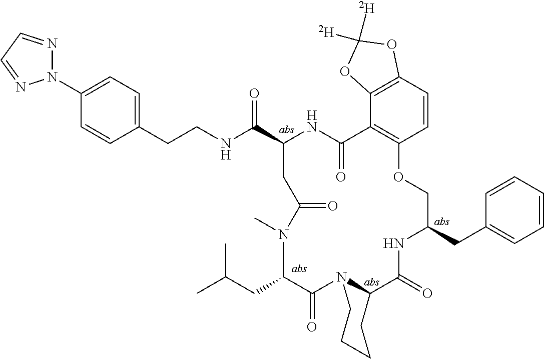 | 653 |
| 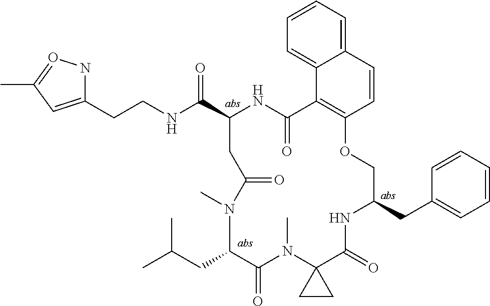 | 654 |
| 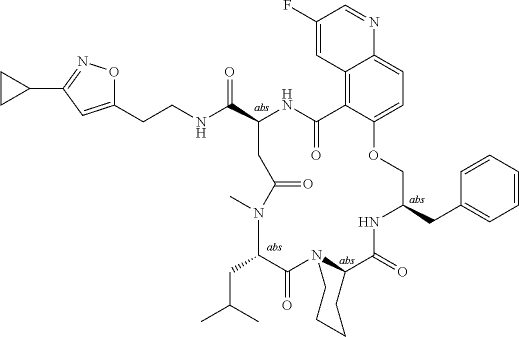 | 655 |
| 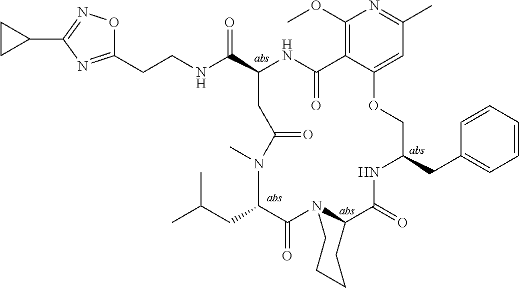 | 656 |
| 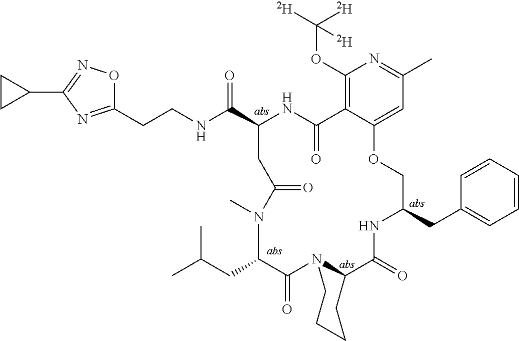 | 657 |
| 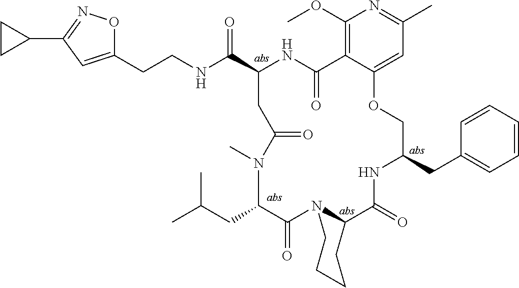 | 658 |
| 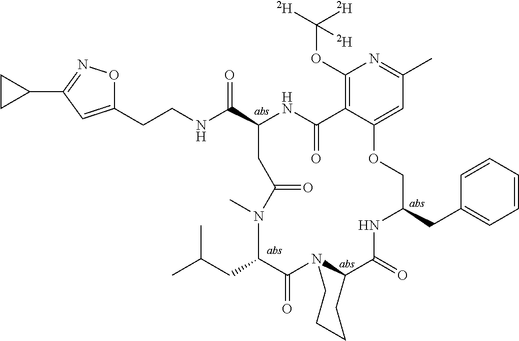 | 659 |
| 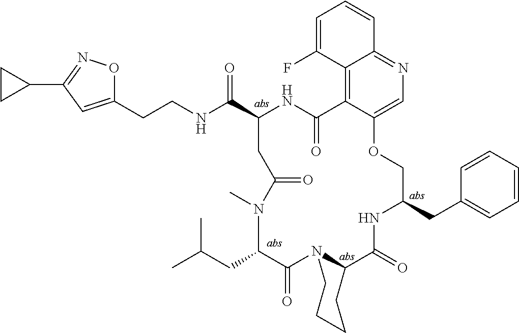 | 660 |
| 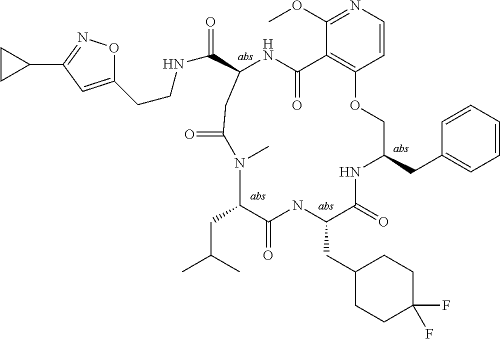 [SMILES: COc1nccc2c1C(=O)N[C@H](C(=O)NCCc1cc(C3CC3)no1)CC(=O)N(C)[C@@H](CC(C)C)C(=O)N[C@@H](CC1CCC(F)(F)CC1)C(=O)N[C@H](Cc1ccccc1)CO2] | 661 |
| 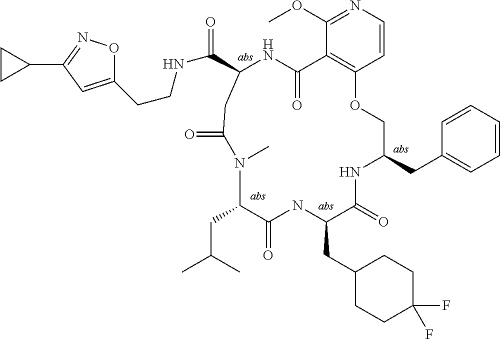 [SMILES: COc1nccc2c1C(=O)N[C@H](C(=O)NCCc1cc(C3CC3)no1)CC(=O)N(C)[C@@H](CC(C)C)C(=O)N[C@H](CC1CCC(F)(F)CC1)C(=O)N[C@H](Cc1ccccc1)CO2] | 662 |
| 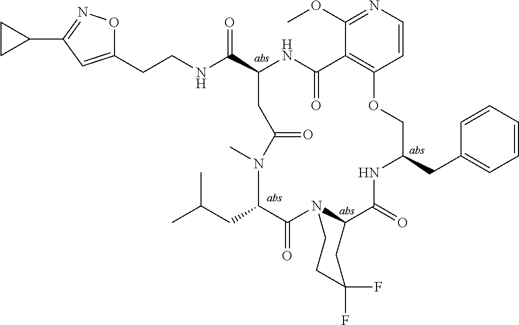 | 663 |
| 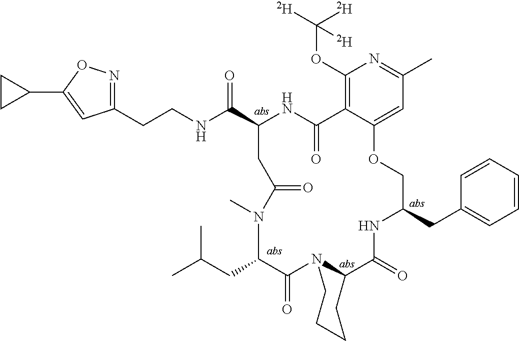 | 664 |
| 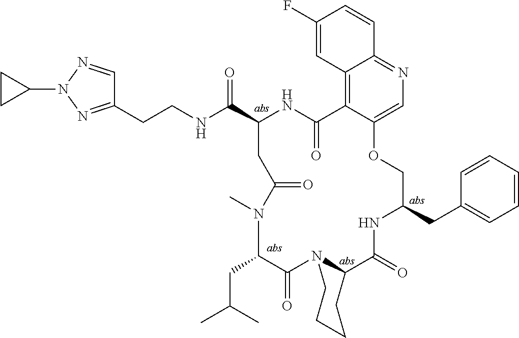 | 665 |
| 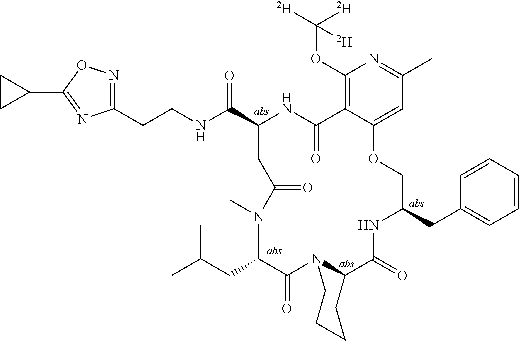 | 666 |
| 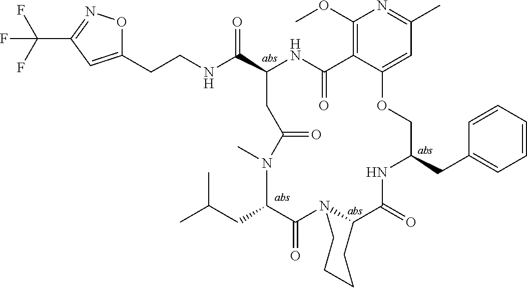 | 667 |
| 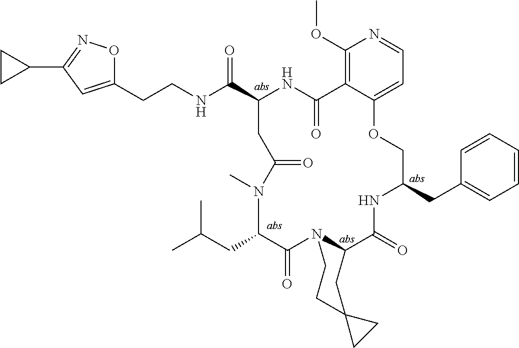 | 668 |
| 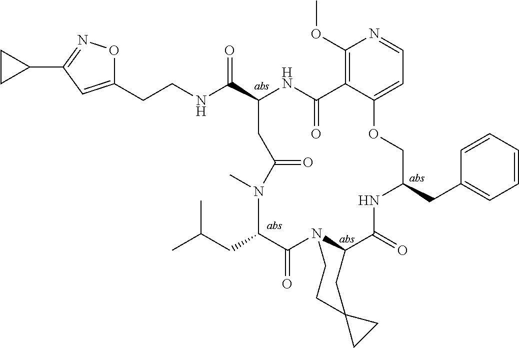 | 669 |
| 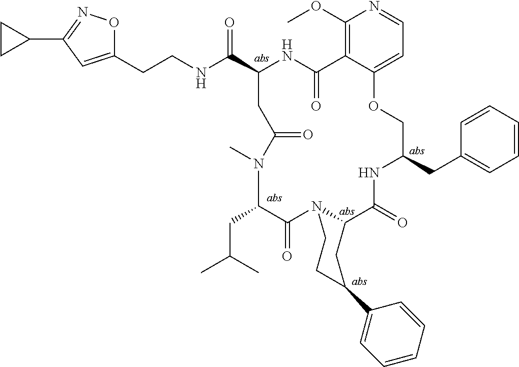 | 670 |
| 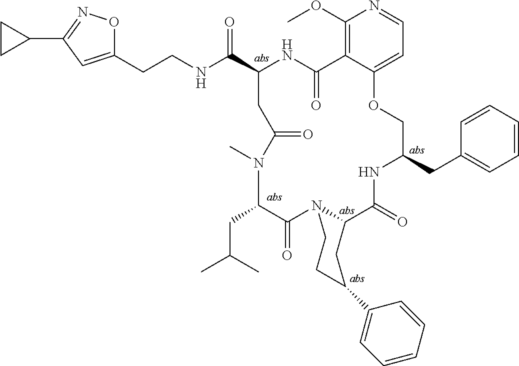 | 671 |
| 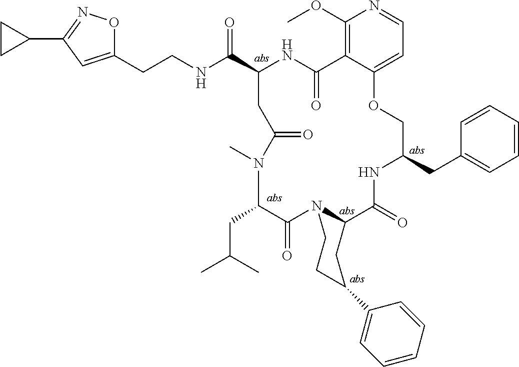 | 672 |
| 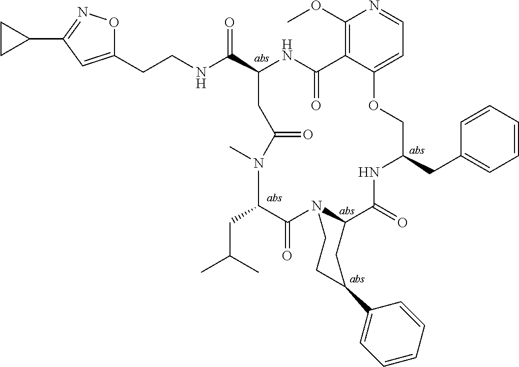 | 673 |
| 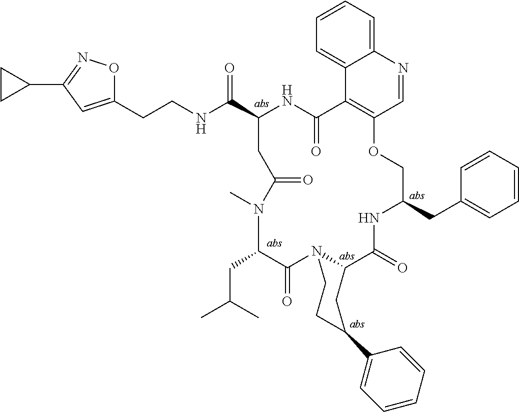 | 674 |
| 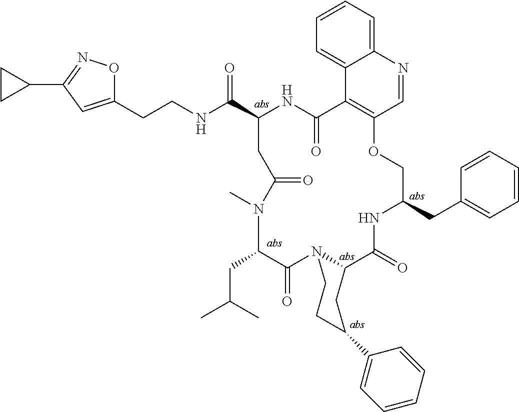 | 675 |
| 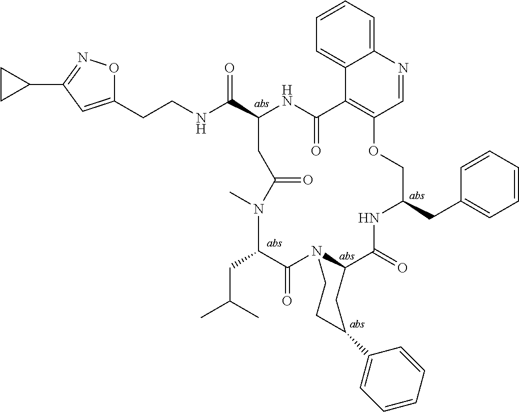 | 676 |
| 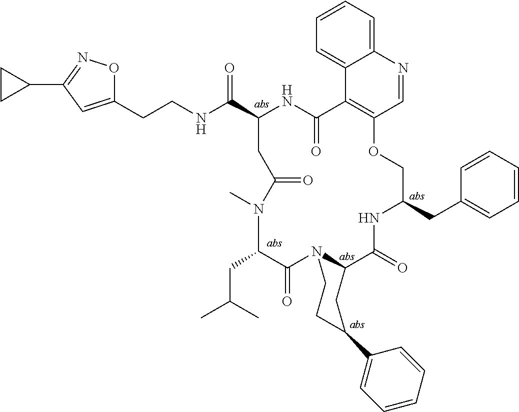 | 677 |
| 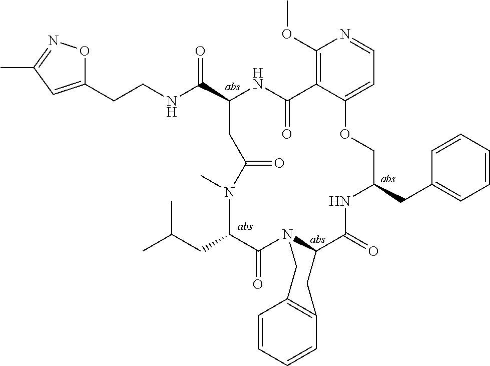 | 678 |
| 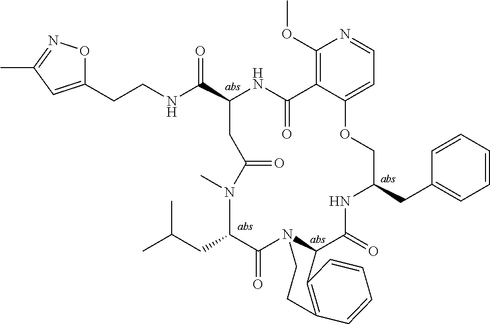 | 679 |
| 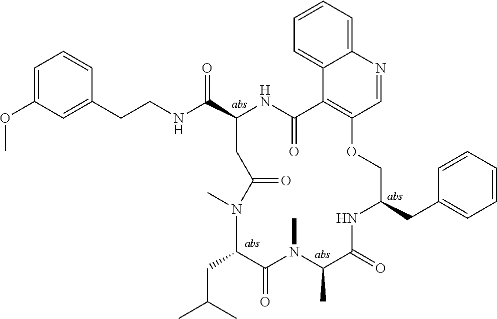 | 680 |
| 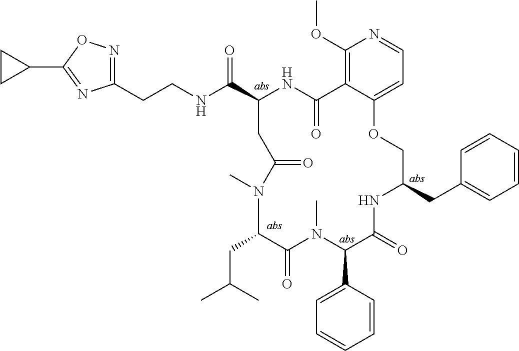 [SMILES: COc1nccc2c1C(=O)N[C@H](C(=O)NCCc1noc(C3CC3)n1)CC(=O)N(C)[C@@H](CC(C)C)C(=O)N(C)[C@H](c1ccccc1)C(=O)N[C@H](Cc1ccccc1)CO2] | 681 |
| 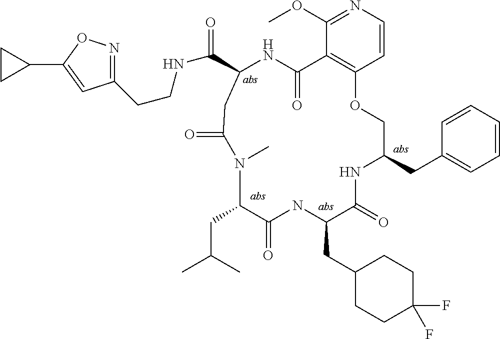 [SMILES: COc1nccc2c1C(=O)N[C@H](C(=O)NCCc1cc(C3CC3)on1)CC(=O)N(C)[C@@H](CC(C)C)C(=O)N[C@H](CC1CCC(F)(F)CC1)C(=O)N[C@H](Cc1ccccc1)CO2] | 682 |
| 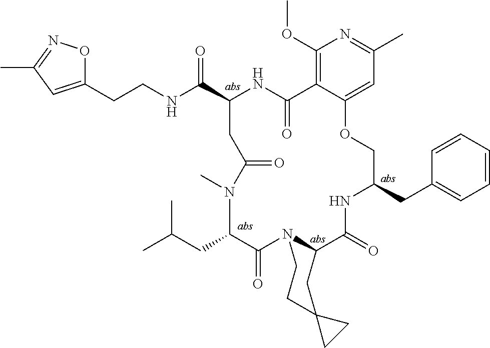 | 683 |
| 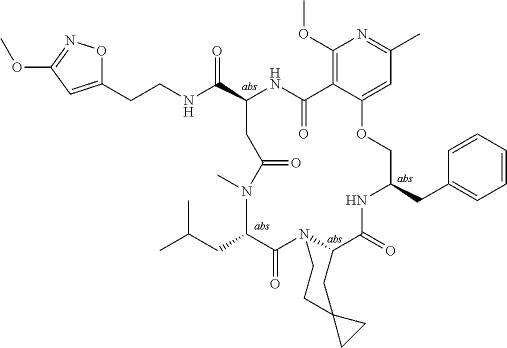 | 684 |
| 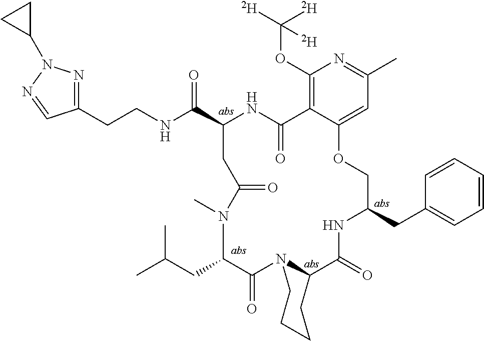 | 685 |
| 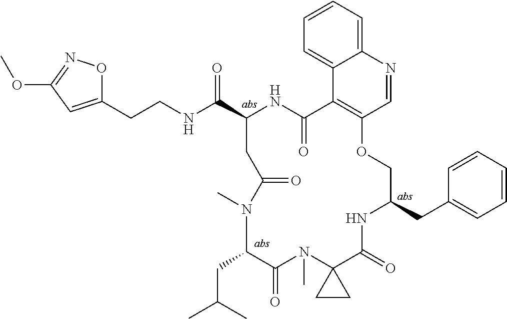 [SMILES: COc1cc(CCNC(=O)[C@@H]2CC(=O)N(C)[C@@H](CC(C)C)C(=O)N(C)C3(CC3)C(=O)N[C@H](Cc3ccccc3)COc3cnc4ccccc4c3C(=O)N2)on1] | 686 |
| 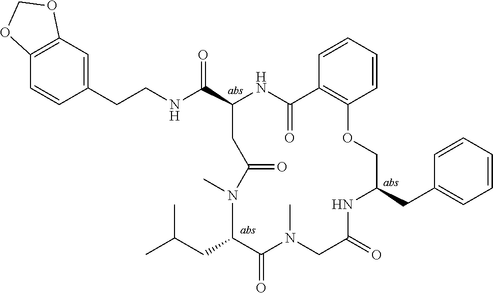 [SMILES: CC(C)C[C@H]1C(=O)N(C)CC(=O)N[C@H](Cc2ccccc2)COc2ccccc2C(=O)N[C@H](C(=O)NCCc2ccc3c(c2)OCO3)CC(=O)N1C] | 687 |
| 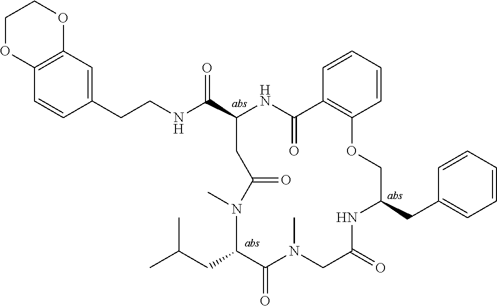 [SMILES: CC(C)C[C@H]1C(=O)N(C)CC(=O)N[C@H](Cc2ccccc2)COc2ccccc2C(=O)N[C@H](C(=O)NCCc2ccc3c(c2)OCCO3)CC(=O)N1C] | 688 |
| 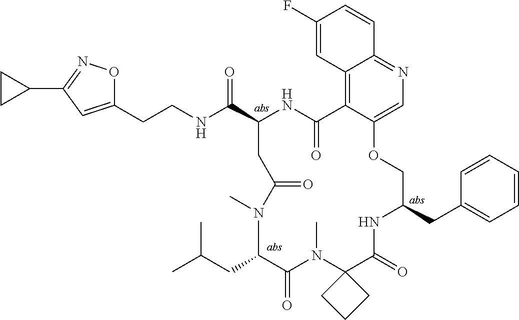 [SMILES: CC(C)C[C@H]1C(=O)N(C)C2(CCC2)C(=O)N[C@H](Cc2ccccc2)COc2cnc3ccc(F)cc3c2C(=O)N[C@H](C(=O)NCCc2cc(C3CC3)no2)CC(=O)N1C] | 689 |
| 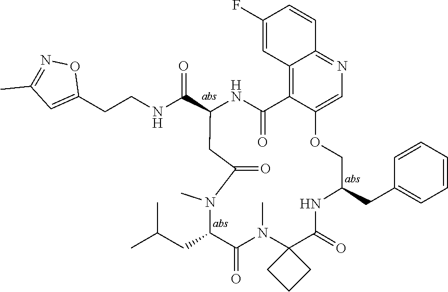 [SMILES: Cc1cc(CCNC(=O)[C@@H]2CC(=O)N(C)[C@@H](CC(C)C)C(=O)N(C)C3(CCC3)C(=O)N[C@H](Cc3ccccc3)COc3cnc4ccc(F)cc4c3C(=O)N2)on1] | 690 |
| 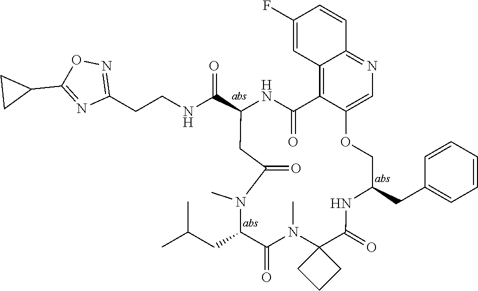 [SMILES: CC(C)C[C@H]1C(=O)N(C)C2(CCC2)C(=O)N[C@H](Cc2ccccc2)COc2cnc3ccc(F)cc3c2C(=O)N[C@H](C(=O)NCCc2noc(C3CC3)n2)CC(=O)N1C] | 691 |
| 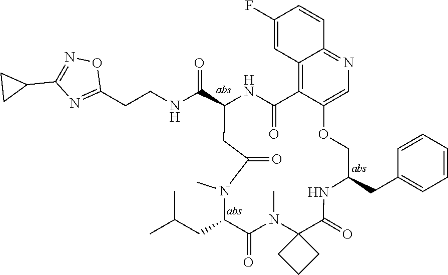 [SMILES: CC(C)C[C@H]1C(=O)N(C)C2(CCC2)C(=O)N[C@H](Cc2ccccc2)COc2cnc3ccc(F)cc3c2C(=O)N[C@H](C(=O)NCCc2nc(C3CC3)no2)CC(=O)N1C] | 692 |
| 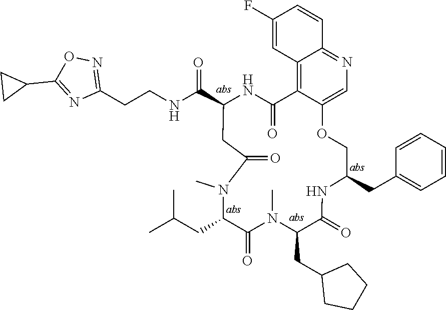 [SMILES: CC(C)C[C@H]1C(=O)N(C)[C@H](CC2CCCC2)C(=O)N[C@H](Cc2ccccc2)COc2cnc3ccc(F)cc3c2C(=O)N[C@H](C(=O)NCCc2noc(C3CC3)n2)CC(=O)N1C] | 693 |
| 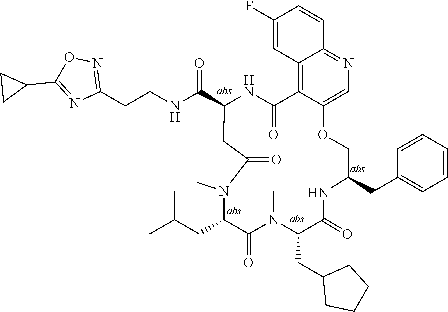 [SMILES: CC(C)C[C@H]1C(=O)N(C)[C@@H](CC2CCCC2)C(=O)N[C@H](Cc2ccccc2)COc2cnc3ccc(F)cc3c2C(=O)N[C@H](C(=O)NCCc2noc(C3CC3)n2)CC(=O)N1C] | 694 |
| 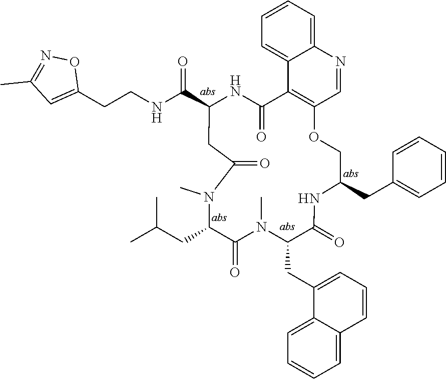 [SMILES: Cc1cc(CCNC(=O)[C@@H]2CC(=O)N(C)[C@@H](CC(C)C)C(=O)N(C)[C@@H](Cc3cccc4ccccc34)C(=O)N[C@H](Cc3ccccc3)COc3cnc4ccccc4c3C(=O)N2)on1] | 695 |
| 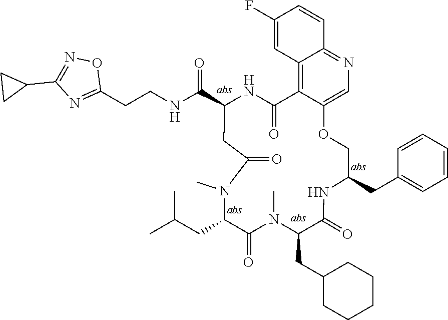 [SMILES: CC(C)C[C@H]1C(=O)N(C)[C@H](CC2CCCCC2)C(=O)N[C@H](Cc2ccccc2)COc2cnc3ccc(F)cc3c2C(=O)N[C@H](C(=O)NCCc2nc(C3CC3)no2)CC(=O)N1C] | 696 |
| 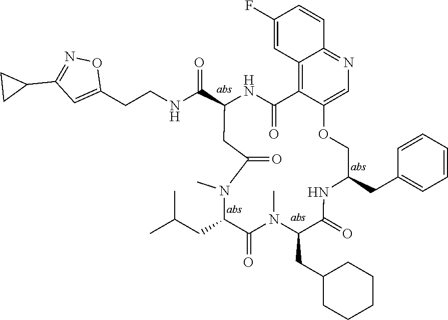 [SMILES: CC(C)C[C@H]1C(=O)N(C)[C@H](CC2CCCCC2)C(=O)N[C@H](Cc2ccccc2)COc2cnc3ccc(F)cc3c2C(=O)N[C@H](C(=O)NCCc2cc(C3CC3)no2)CC(=O)N1C] | 697 |
| 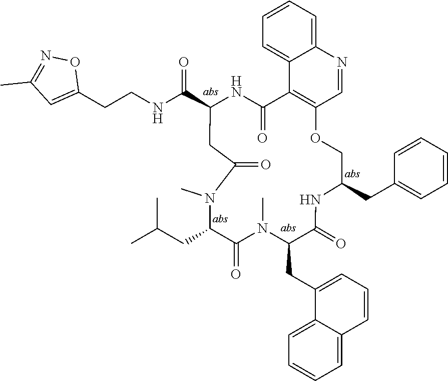 [SMILES: Cc1cc(CCNC(=O)[C@@H]2CC(=O)N(C)[C@@H](CC(C)C)C(=O)N(C)[C@H](Cc3cccc4ccccc34)C(=O)N[C@H](Cc3ccccc3)COc3cnc4ccccc4c3C(=O)N2)on1] | 698 |
| 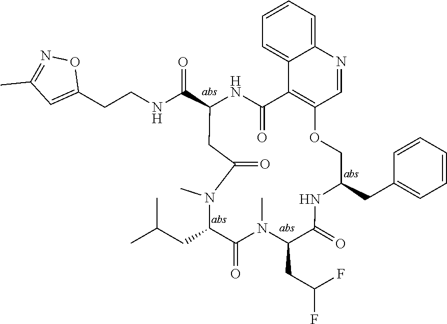 [SMILES: Cc1cc(CCNC(=O)[C@@H]2CC(=O)N(C)[C@@H](CC(C)C)C(=O)N(C)[C@H](CC(F)F)C(=O)N[C@H](Cc3ccccc3)COc3cnc4ccccc4c3C(=O)N2)on1] | 699 |
| 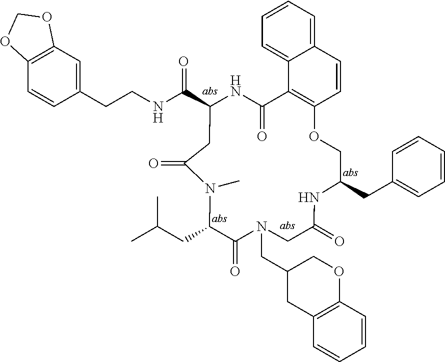 [SMILES: CC(C)C[C@H]1C(=O)N(CC2COc3ccccc3C2)CC(=O)N[C@H](Cc2ccccc2)COc2ccc3ccccc3c2C(=O)N[C@H](C(=O)NCCc2ccc3c(c2)OCO3)CC(=O)N1C] | 700 |
| 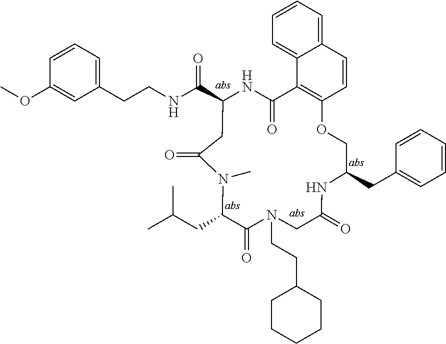 [SMILES: COc1cccc(CCNC(=O)[C@@H]2CC(=O)N(C)[C@@H](CC(C)C)C(=O)N(CCC3CCCCC3)CC(=O)N[C@H](Cc3ccccc3)COc3ccc4ccccc4c3C(=O)N2)c1] | 701 |
| 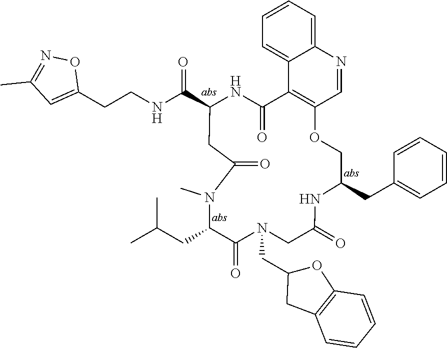 | 702 |
| 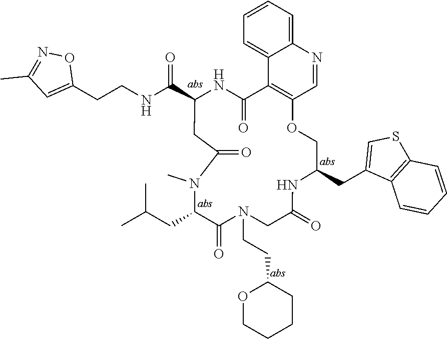 [SMILES: Cc1cc(CCNC(=O)[C@@H]2CC(=O)N(C)[C@@H](CC(C)C)C(=O)N(CC[C@@H]3CCCCO3)CC(=O)N[C@H](Cc3csc4ccccc34)COc3cnc4ccccc4c3C(=O)N2)on1] | 703 |
| 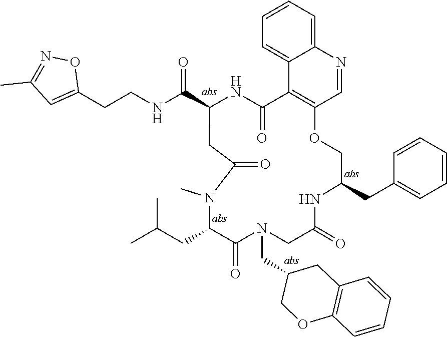 [SMILES: Cc1cc(CCNC(=O)[C@@H]2CC(=O)N(C)[C@@H](CC(C)C)C(=O)N(C[C@H]3COc4ccccc4C3)CC(=O)N[C@H](Cc3ccccc3)COc3cnc4ccccc4c3C(=O)N2)on1] | 704 |
| 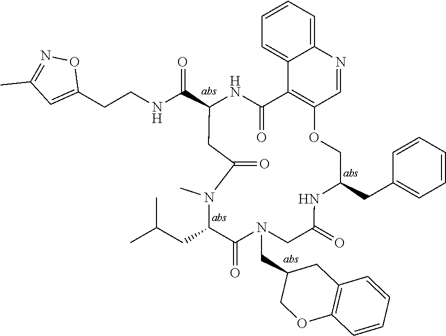 [SMILES: Cc1cc(CCNC(=O)[C@@H]2CC(=O)N(C)[C@@H](CC(C)C)C(=O)N(C[C@@H]3COc4ccccc4C3)CC(=O)N[C@H](Cc3ccccc3)COc3cnc4ccccc4c3C(=O)N2)on1] | 705 |
| 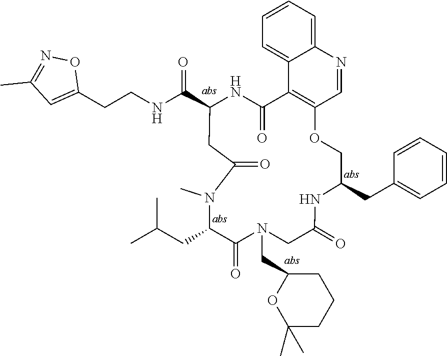 [SMILES: Cc1cc(CCNC(=O)[C@@H]2CC(=O)N(C)[C@@H](CC(C)C)C(=O)N(C[C@H]3CCCC(C)(C)O3)CC(=O)N[C@H](Cc3ccccc3)COc3cnc4ccccc4c3C(=O)N2)on1] | 706 |
| 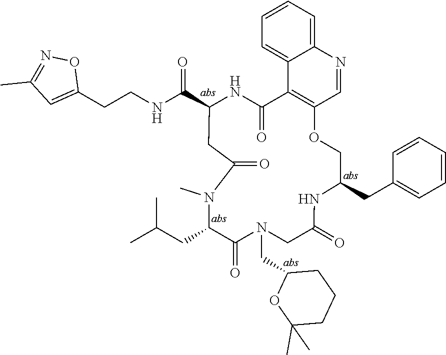 [SMILES: Cc1cc(CCNC(=O)[C@@H]2CC(=O)N(C)[C@@H](CC(C)C)C(=O)N(C[C@@H]3CCCC(C)(C)O3)CC(=O)N[C@H](Cc3ccccc3)COc3cnc4ccccc4c3C(=O)N2)on1] | 707 |
| 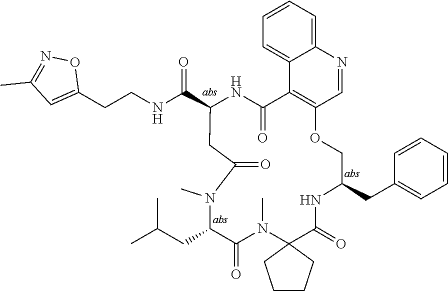 [SMILES: Cc1cc(CCNC(=O)[C@@H]2CC(=O)N(C)[C@@H](CC(C)C)C(=O)N(C)C3(CCCC3)C(=O)N[C@H](Cc3ccccc3)COc3cnc4ccccc4c3C(=O)N2)on1] | 708 |
| 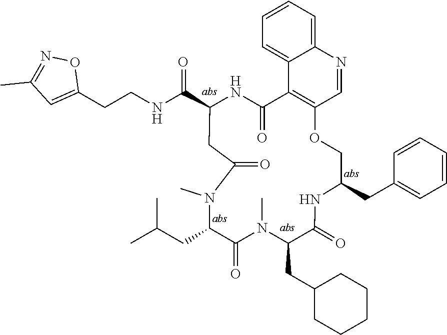 [SMILES: Cc1cc(CCNC(=O)[C@@H]2CC(=O)N(C)[C@@H](CC(C)C)C(=O)N(C)[C@H](CC3CCCCC3)C(=O)N[C@H](Cc3ccccc3)COc3cnc4ccccc4c3C(=O)N2)on1] | 709 |
| 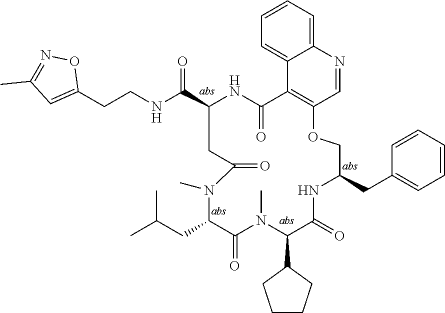 [SMILES: Cc1cc(CCNC(=O)[C@@H]2CC(=O)N(C)[C@@H](CC(C)C)C(=O)N(C)[C@H](C3CCCC3)C(=O)N[C@H](Cc3ccccc3)COc3cnc4ccccc4c3C(=O)N2)on1] | 710 |
| 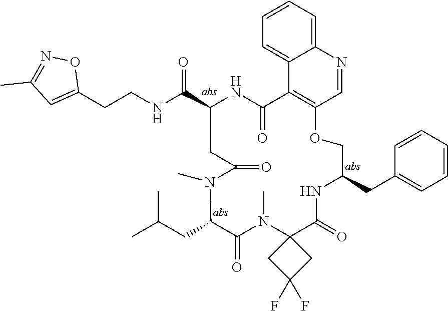 [SMILES: Cc1cc(CCNC(=O)[C@@H]2CC(=O)N(C)[C@@H](CC(C)C)C(=O)N(C)C3(CC(F)(F)C3)C(=O)N[C@H](Cc3ccccc3)COc3cnc4ccccc4c3C(=O)N2)on1] | 711 |
| 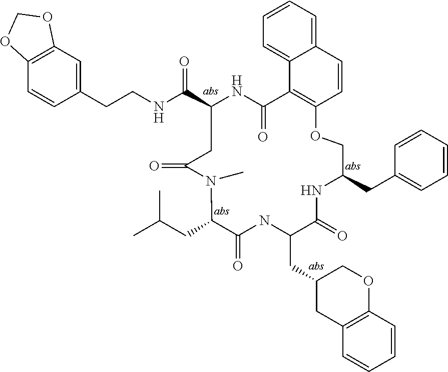 [SMILES: CC(C)C[C@H]1C(=O)NC(C[C@@H]2COc3ccccc3C2)C(=O)N[C@H](Cc2ccccc2)COc2ccc3ccccc3c2C(=O)N[C@H](C(=O)NCCc2ccc3c(c2)OCO3)CC(=O)N1C] | 712 |
| 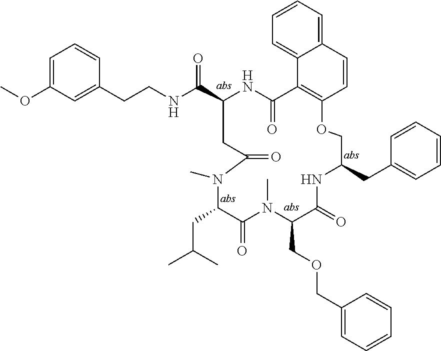 [SMILES: COc1cccc(CCNC(=O)[C@@H]2CC(=O)N(C)[C@@H](CC(C)C)C(=O)N(C)[C@H](COCc3ccccc3)C(=O)N[C@H](Cc3ccccc3)COc3ccc4ccccc4c3C(=O)N2)c1] | 713 |
| 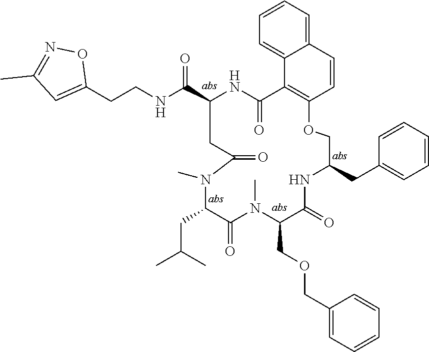 [SMILES: Cc1cc(CCNC(=O)[C@@H]2CC(=O)N(C)[C@@H](CC(C)C)C(=O)N(C)[C@H](COCc3ccccc3)C(=O)N[C@H](Cc3ccccc3)COc3ccc4ccccc4c3C(=O)N2)on1] | 714 |
| 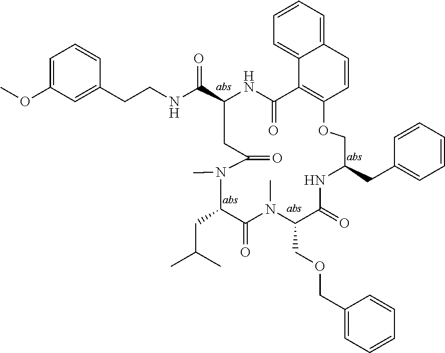 [SMILES: COc1cccc(CCNC(=O)[C@@H]2CC(=O)N(C)[C@@H](CC(C)C)C(=O)N(C)[C@@H](COCc3ccccc3)C(=O)N[C@H](Cc3ccccc3)COc3ccc4ccccc4c3C(=O)N2)c1] | 715 |
| 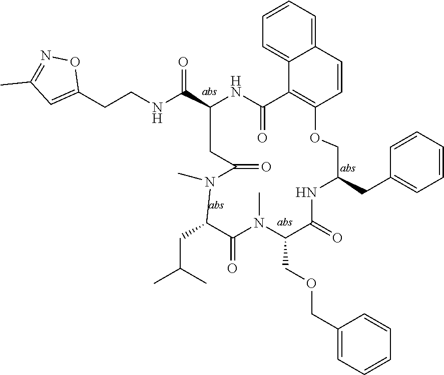 [SMILES: Cc1cc(CCNC(=O)[C@@H]2CC(=O)N(C)[C@@H](CC(C)C)C(=O)N(C)[C@@H](COCc3ccccc3)C(=O)N[C@H](Cc3ccccc3)COc3ccc4ccccc4c3C(=O)N2)on1] | 716 |
| 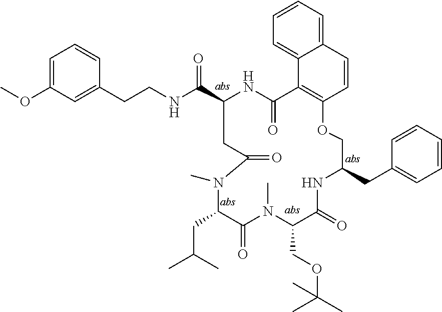 [SMILES: COc1cccc(CCNC(=O)[C@@H]2CC(=O)N(C)[C@@H](CC(C)C)C(=O)N(C)[C@@H](COC(C)(C)C)C(=O)N[C@H](Cc3ccccc3)COc3ccc4ccccc4c3C(=O)N2)c1] | 717 |
| 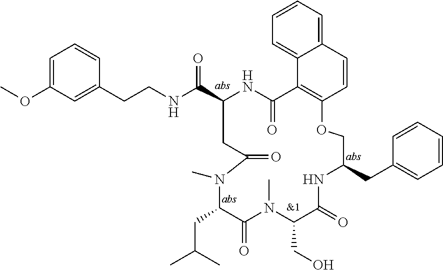 [SMILES: COc1cccc(CCNC(=O)[C@@H]2CC(=O)N(C)[C@@H](CC(C)C)C(=O)N(C)[C@@H](CO)C(=O)N[C@H](Cc3ccccc3)COc3ccc4ccccc4c3C(=O)N2)c1] | 718 |
| 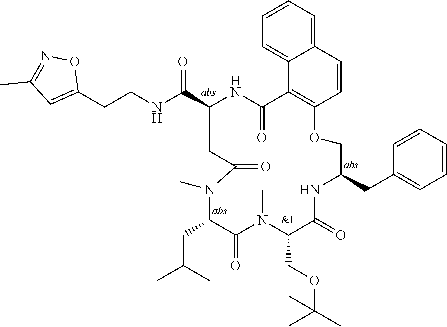 [SMILES: Cc1cc(CCNC(=O)[C@@H]2CC(=O)N(C)[C@@H](CC(C)C)C(=O)N(C)[C@@H](COC(C)(C)C)C(=O)N[C@H](Cc3ccccc3)COc3ccc4ccccc4c3C(=O)N2)on1] | 719 |
| 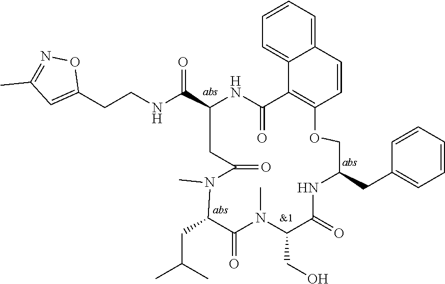 [SMILES: Cc1cc(CCNC(=O)[C@@H]2CC(=O)N(C)[C@@H](CC(C)C)C(=O)N(C)[C@@H](CO)C(=O)N[C@H](Cc3ccccc3)COc3ccc4ccccc4c3C(=O)N2)on1] | 720 |
| 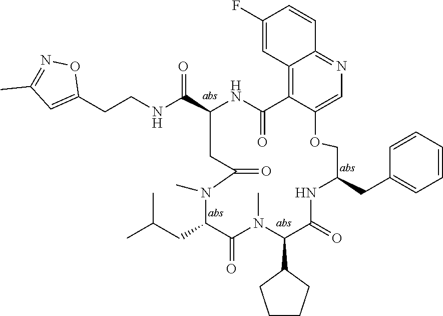 [SMILES: Cc1cc(CCNC(=O)[C@@H]2CC(=O)N(C)[C@@H](CC(C)C)C(=O)N(C)[C@H](C3CCCC3)C(=O)N[C@H](Cc3ccccc3)COc3cnc4ccc(F)cc4c3C(=O)N2)on1] | 721 |
| 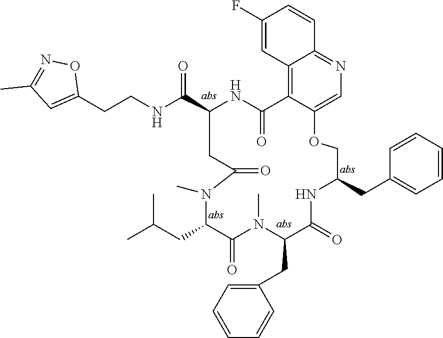 [SMILES: Cc1cc(CCNC(=O)[C@@H]2CC(=O)N(C)[C@@H](CC(C)C)C(=O)N(C)[C@H](Cc3ccccc3)C(=O)N[C@H](Cc3ccccc3)COc3cnc4ccc(F)cc4c3C(=O)N2)on1] | 722 |
| 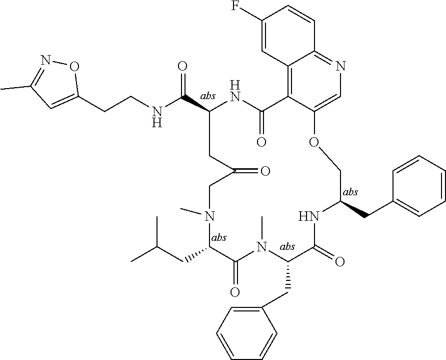 [SMILES: Cc1cc(CCNC(=O)[C@@H]2CC(=O)CN(C)[C@@H](CC(C)C)C(=O)N(C)[C@@H](Cc3ccccc3)C(=O)N[C@H](Cc3ccccc3)COc3cnc4ccc(F)cc4c3C(=O)N2)on1] | 723 |
| 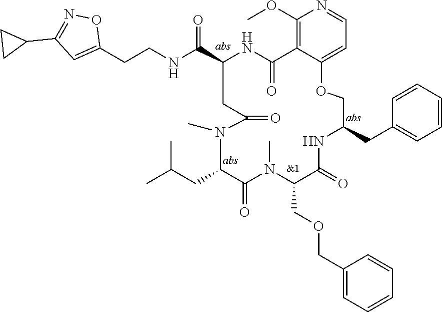 [SMILES: COc1nccc2c1C(=O)N[C@H](C(=O)NCCc1cc(C3CC3)no1)CC(=O)N(C)[C@@H](CC(C)C)C(=O)N(C)[C@@H](COCc1ccccc1)C(=O)N[C@H](Cc1ccccc1)CO2] | 724 |
| 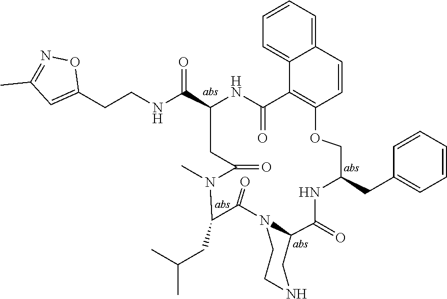 | 725 |
| 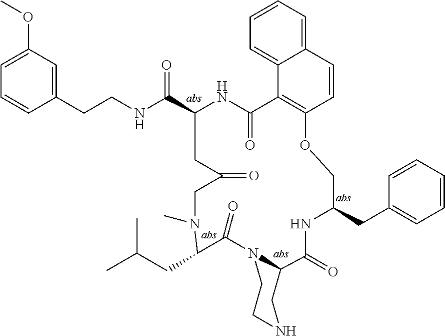 | 726 |
| 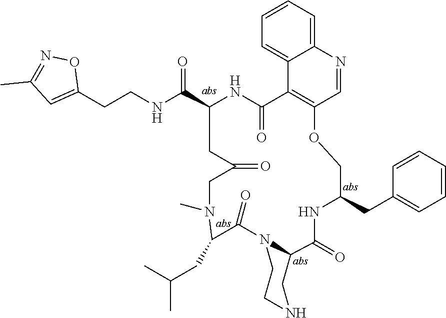 | 727 |
| 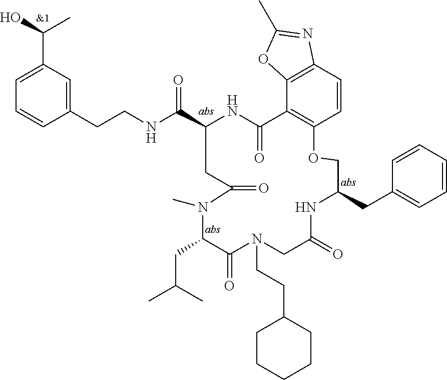 [SMILES: Cc1nc2ccc3c(c2o1)C(=O)N[C@H](C(=O)NCCc1cccc([C@H](C)O)c1)CC(=O)N(C)[C@@H](CC(C)C)C(=O)N(CCC1CCCCC1)CC(=O)N[C@H](Cc1ccccc1)CO3] | 728 |
| 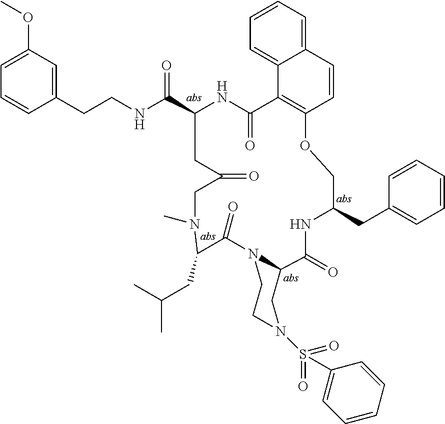 | 729 |
| 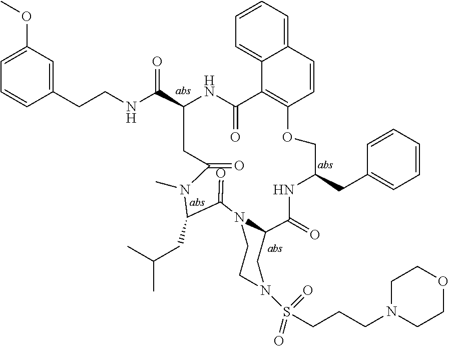 | 730 |
| 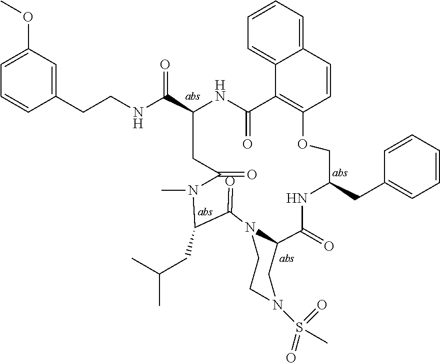 | 731 |
| 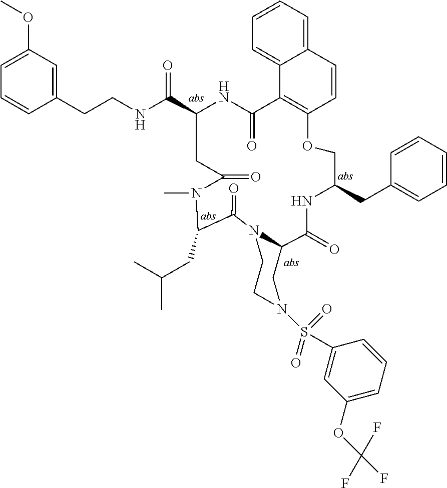 | 732 |
| 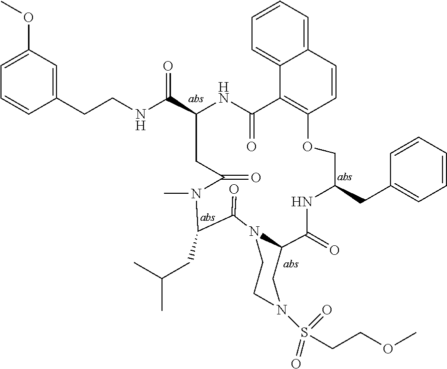 | 733 |
| 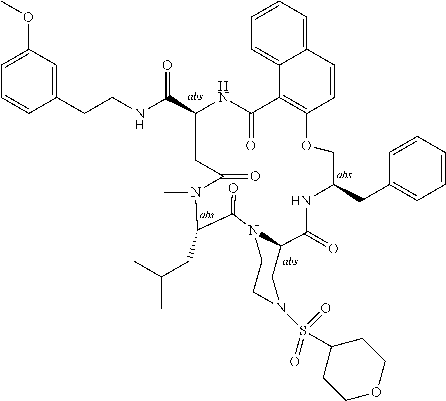 | 734 |
| 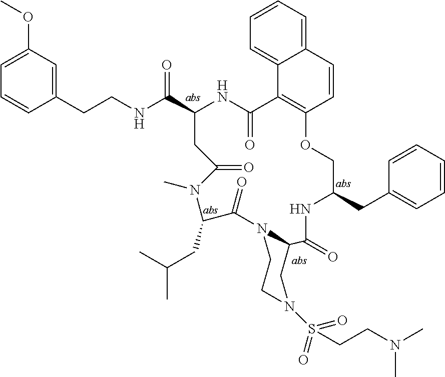 | 735 |
| 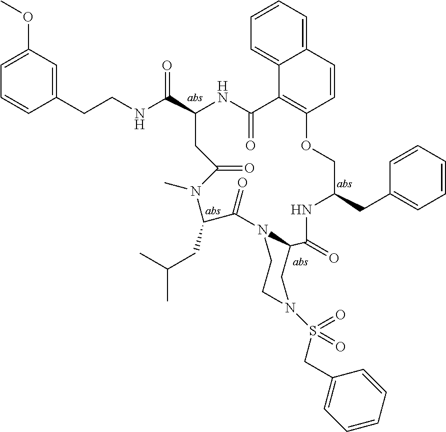 | 736 |
| 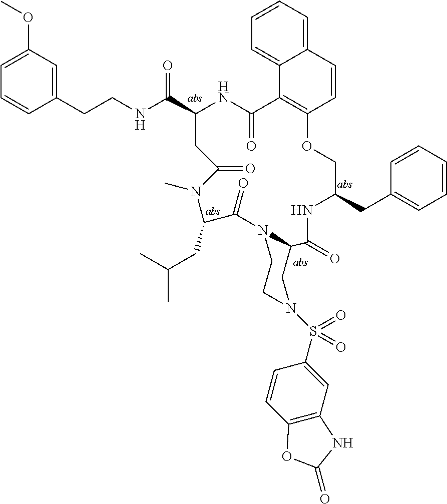 | 737 |
| 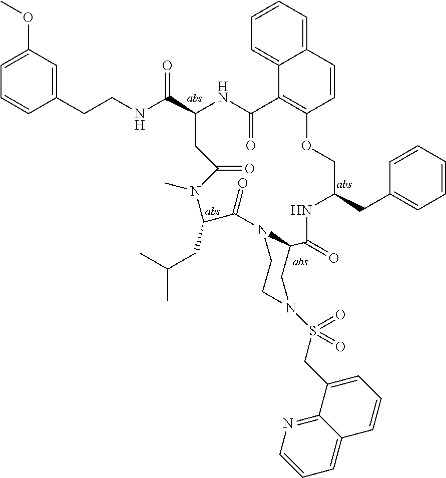 | 738 |
| 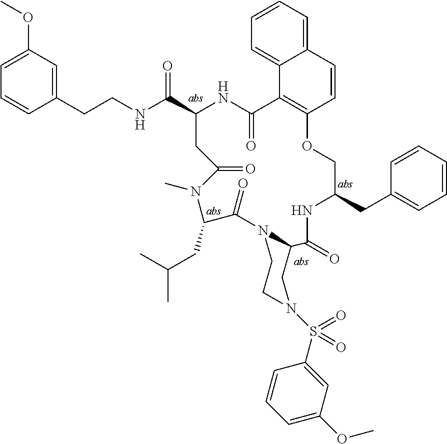 | 739 |
| 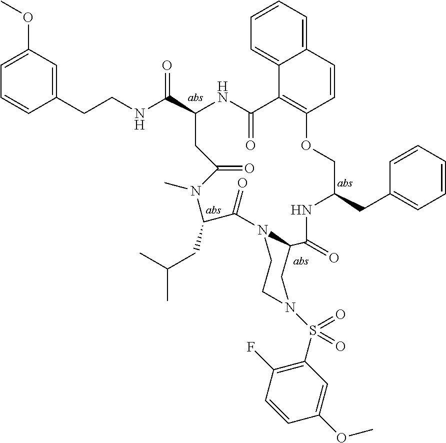 | 740 |
| 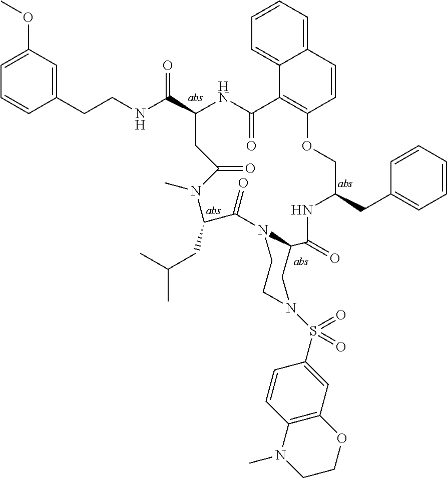 | 741 |
| 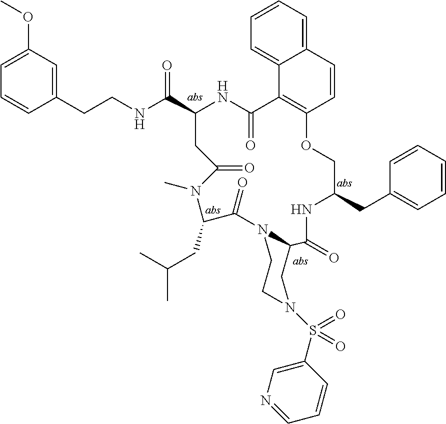 | 742 |
| 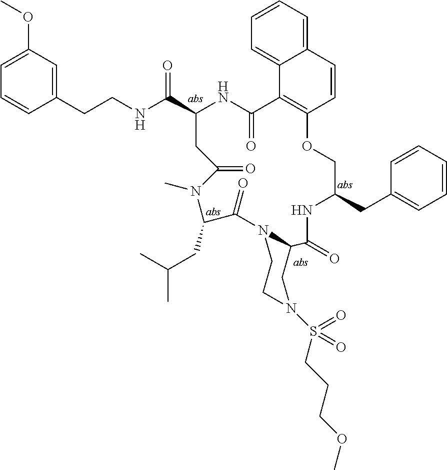 | 743 |
| 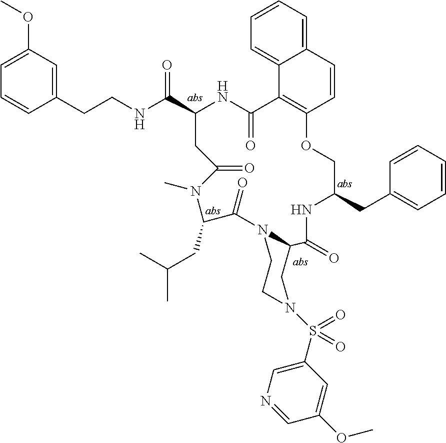 | 744 |
| 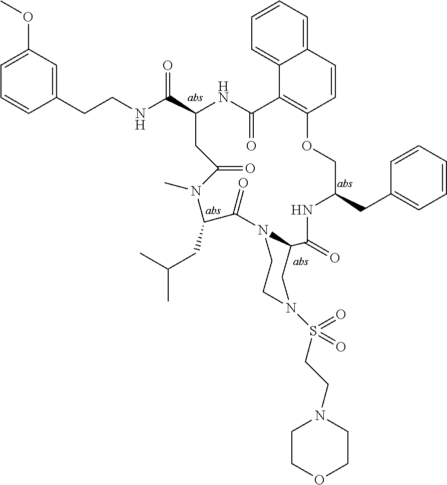 | 745 |
| 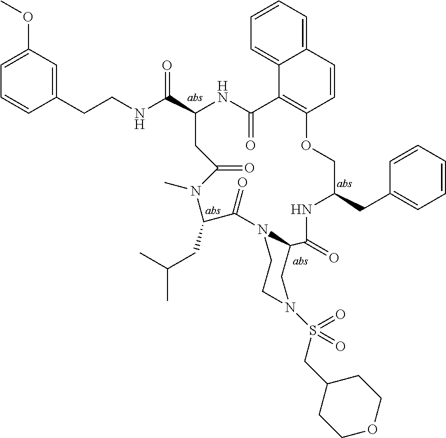 | 746 |
| 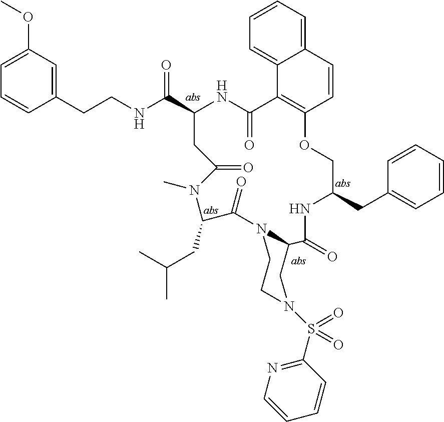 | 747 |
| 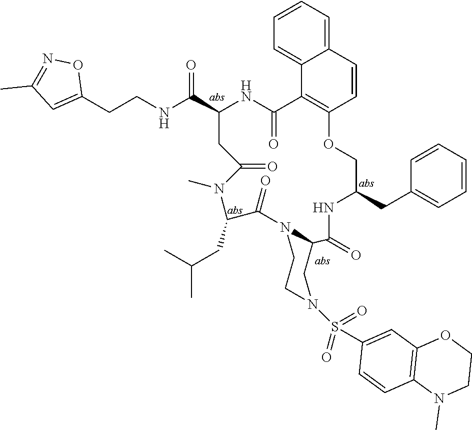 | 748 |
| 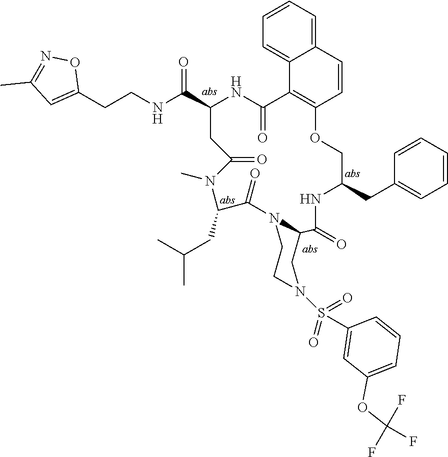 | 749 |
| 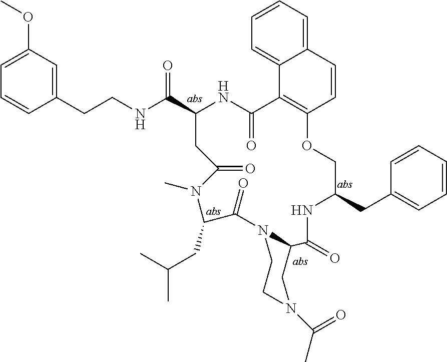 | 750 |
| 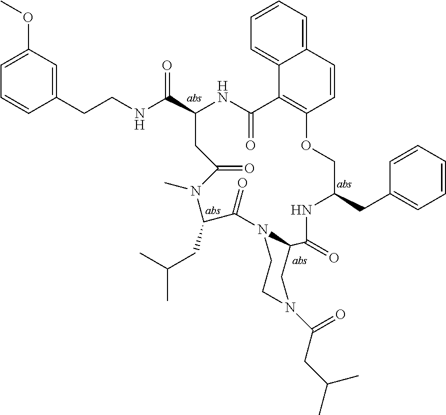 | 751 |
| 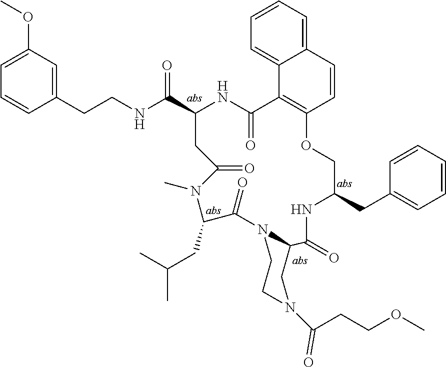 | 752 |
| 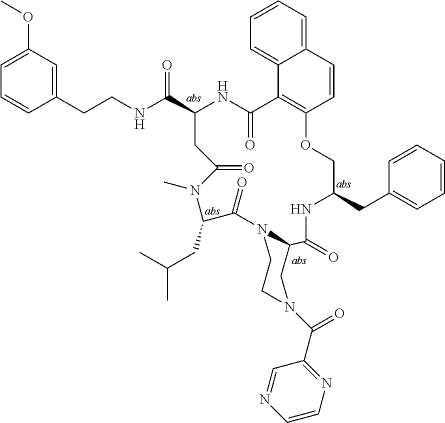 | 753 |
| 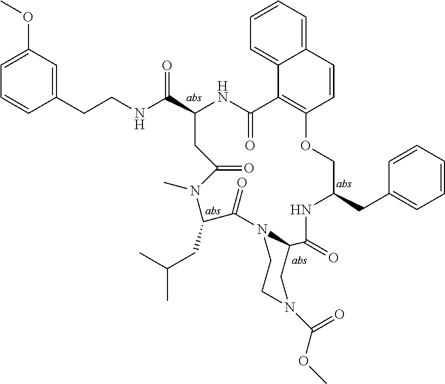 | 754 |
| 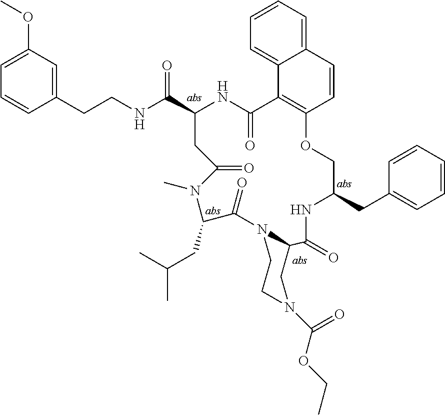 | 755 |
| 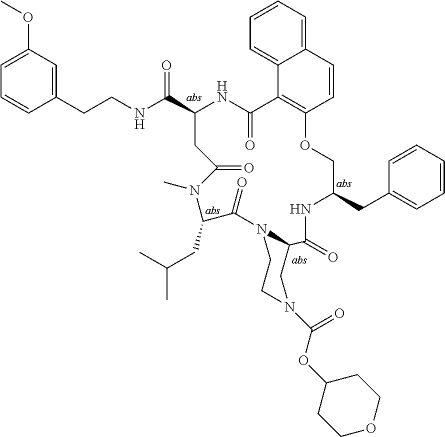 | 756 |
| 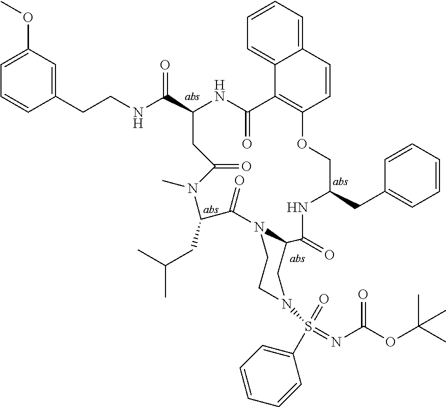 | 757 |
| 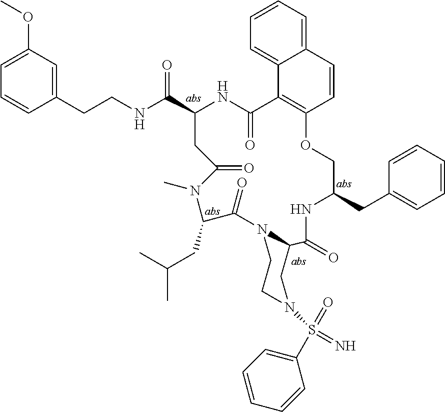 | 758 |
| 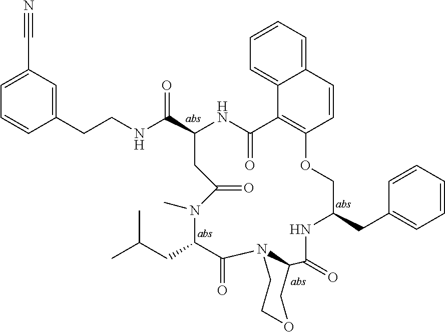 | 759 |
| 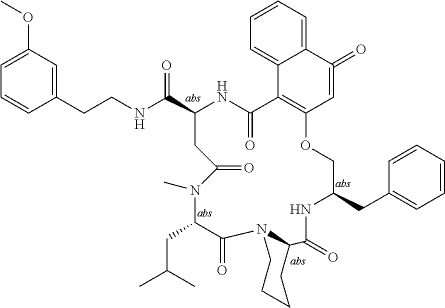 | 760 |
| 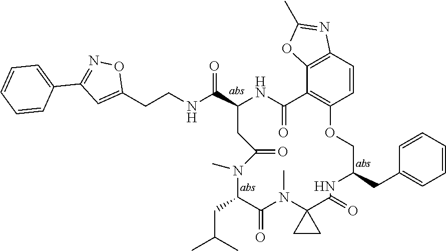 [SMILES: Cc1nc2ccc3c(c2o1)C(=O)N[C@H](C(=O)NCCc1cc(-c2ccccc2)no1)CC(=O)N(C)[C@@H](CC(C)C)C(=O)N(C)C1(CC1)C(=O)N[C@H](Cc1ccccc1)CO3] | 761 |
| 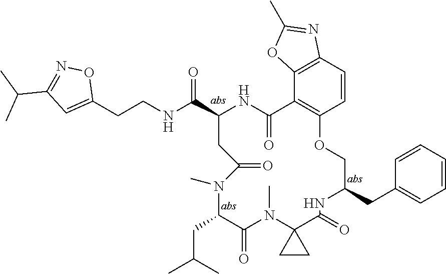 [SMILES: Cc1nc2ccc3c(c2o1)C(=O)N[C@H](C(=O)NCCc1cc(C(C)C)no1)CC(=O)N(C)[C@@H](CC(C)C)C(=O)N(C)C1(CC1)C(=O)N[C@H](Cc1ccccc1)CO3] | 762 |
| 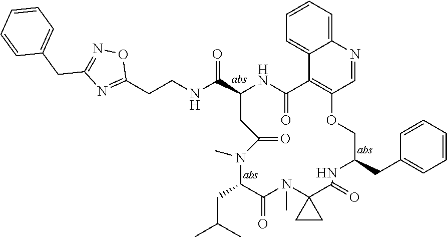 [SMILES: CC(C)C[C@H]1C(=O)N(C)C2(CC2)C(=O)N[C@H](Cc2ccccc2)COc2cnc3ccccc3c2C(=O)N[C@H](C(=O)NCCc2nc(Cc3ccccc3)no2)CC(=O)N1C] | 763 |
| 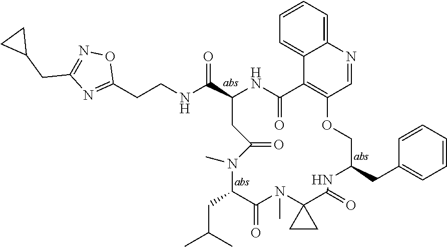 [SMILES: CC(C)C[C@H]1C(=O)N(C)C2(CC2)C(=O)N[C@H](Cc2ccccc2)COc2cnc3ccccc3c2C(=O)N[C@H](C(=O)NCCc2nc(CC3CC3)no2)CC(=O)N1C] | 764 |

TABLE 4
Structures of compounds of Example 765 to 829

| | |
|---|---|
| 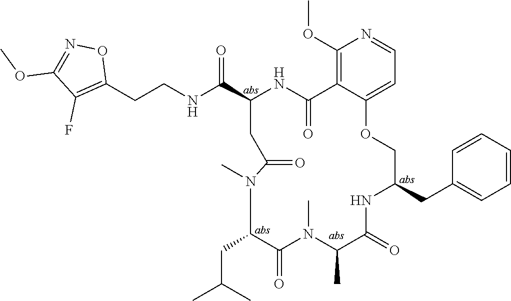 [SMILES: COc1noc(CCNC(=O)[C@@H]2CC(=O)N(C)[C@@H](CC(C)C)C(=O)N(C)[C@H](C)C(=O)N[C@H](Cc3ccccc3)COc3ccnc(OC)c3C(=O)N2)c1F] | 765 |
| 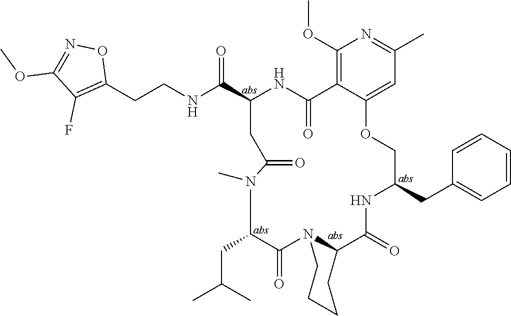 | 766 |
| 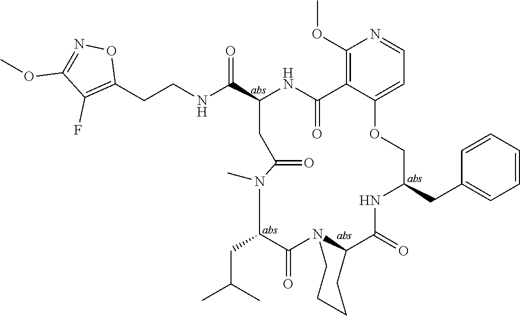 | 767 |
| 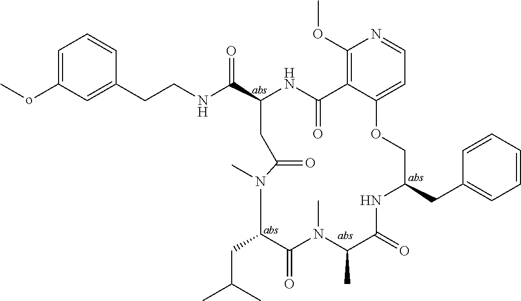 | 768 |
| 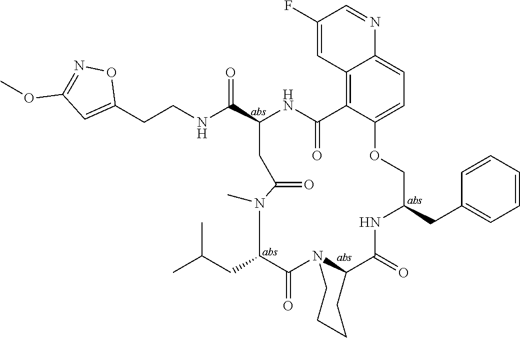 | 769 |
| 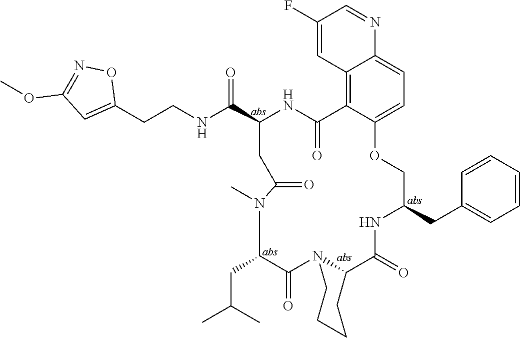 | 770 |
| 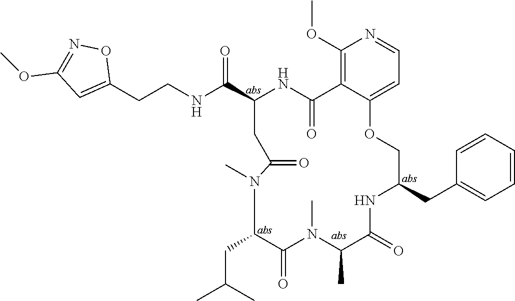 [SMILES: COc1cc(CCNC(=O)[C@@H]2CC(=O)N(C)[C@@H](CC(C)C)C(=O)N(C)[C@H](C)C(=O)N[C@H](Cc3ccccc3)COc3ccnc(OC)c3C(=O)N2)on1] | 771 |
| 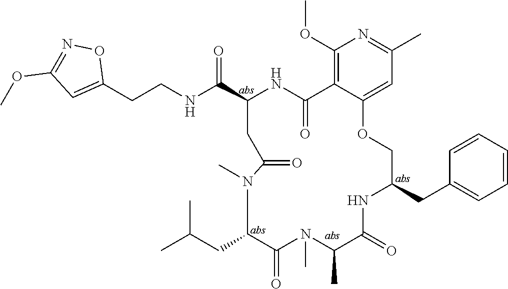 [SMILES: COc1cc(CCNC(=O)[C@@H]2CC(=O)N(C)[C@@H](CC(C)C)C(=O)N(C)[C@H](C)C(=O)N[C@H](Cc3ccccc3)COc3cc(C)nc(OC)c3C(=O)N2)on1] | 772 |
| 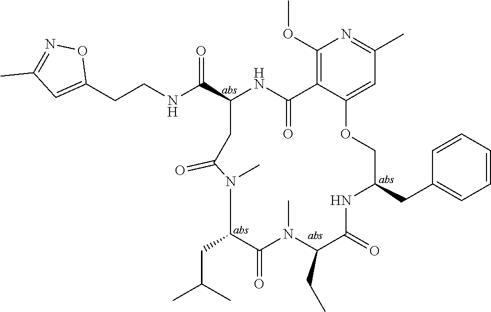 [SMILES: CC[C@@H]1C(=O)N[C@H](Cc2ccccc2)COc2cc(C)nc(OC)c2C(=O)N[C@H](C(=O)NCCc2cc(C)no2)CC(=O)N(C)[C@@H](CC(C)C)C(=O)N1C] | 773 |
| 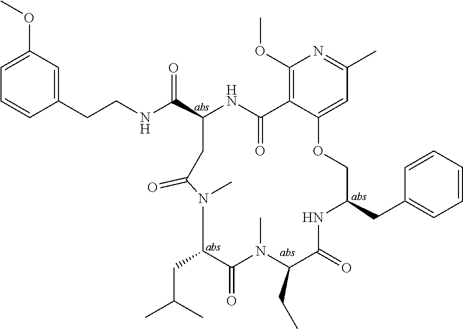 [SMILES: CC[C@@H]1C(=O)N[C@H](Cc2ccccc2)COc2cc(C)nc(OC)c2C(=O)N[C@H](C(=O)NCCc2cccc(OC)c2)CC(=O)N(C)[C@@H](CC(C)C)C(=O)N1C] | 774 |
| 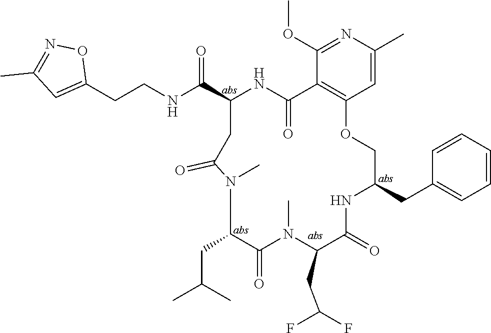 [SMILES: COc1nc(C)cc2c1C(=O)N[C@H](C(=O)NCCc1cc(C)no1)CC(=O)N(C)[C@@H](CC(C)C)C(=O)N(C)[C@H](CC(F)F)C(=O)N[C@H](Cc1ccccc1)CO2] | 775 |
| 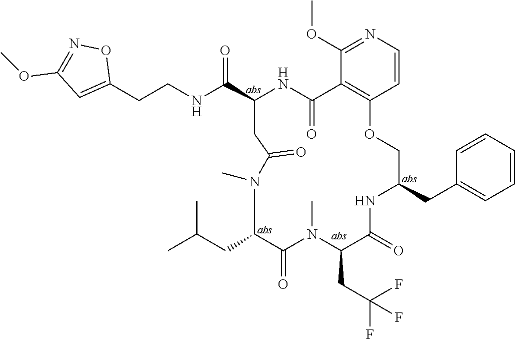 [SMILES: COc1cc(CCNC(=O)[C@@H]2CC(=O)N(C)[C@@H](CC(C)C)C(=O)N(C)[C@H](CC(F)(F)F)C(=O)N[C@H](Cc3ccccc3)COc3ccnc(OC)c3C(=O)N2)on1] | 776 |
| 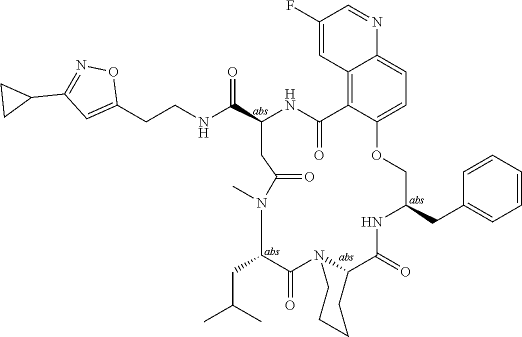 | 777 |
| 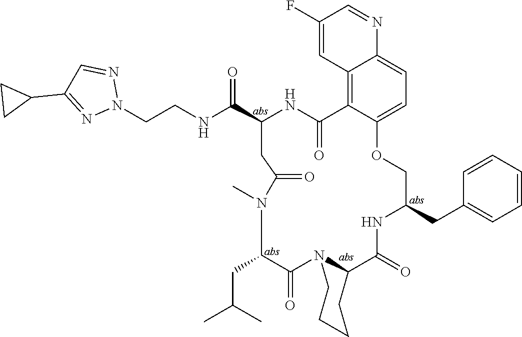 | 778 |
| 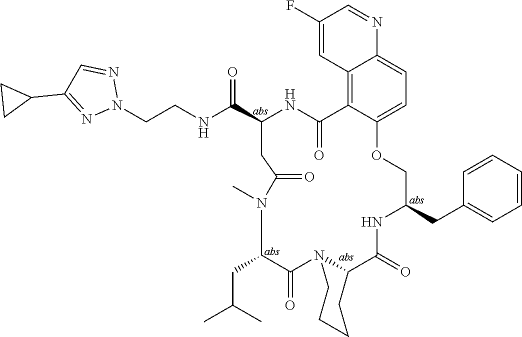 | 779 |
| 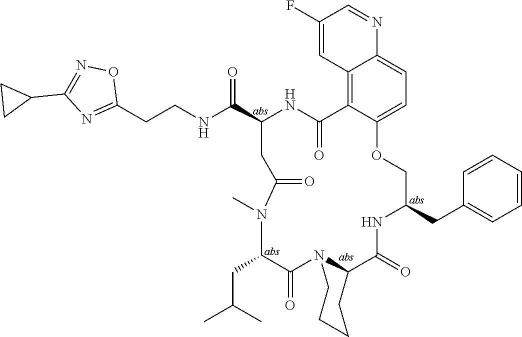 | 780 |
| 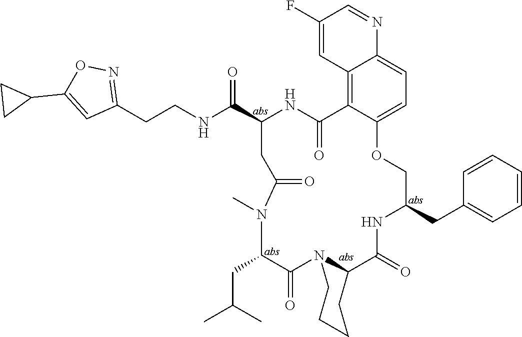 | 781 |
| 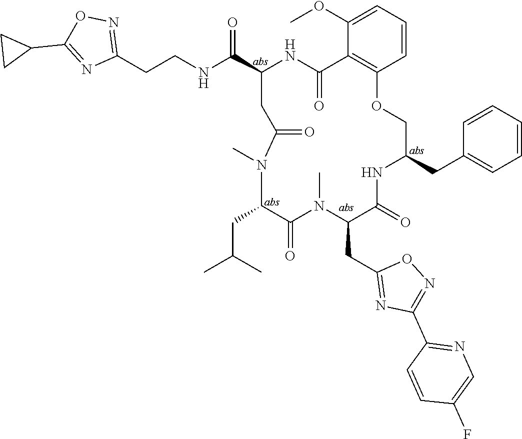 [SMILES: COc1cccc2c1C(=O)N[C@H](C(=O)NCCc1noc(C3CC3)n1)CC(=O)N(C)[C@@H](CC(C)C)C(=O)N(C)[C@H](Cc1nc(-c3ccc(F)cn3)no1)C(=O)N[C@H](Cc1ccccc1)CO2] | 782 |
| 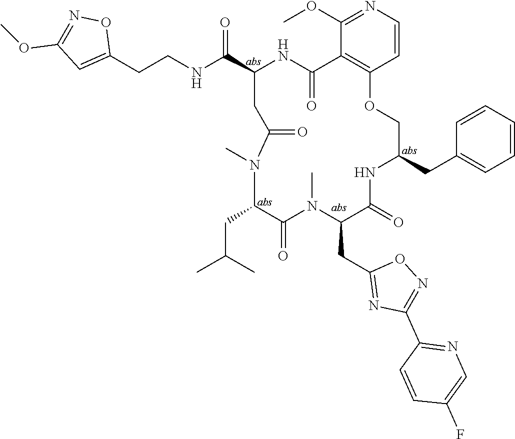 [SMILES: COc1cc(CCNC(=O)[C@@H]2CC(=O)N(C)[C@@H](CC(C)C)C(=O)N(C)[C@H](Cc3nc(-c4ccc(F)cn4)no3)C(=O)N[C@H](Cc3ccccc3)COc3ccnc(OC)c3C(=O)N2)on1] | 783 |
| 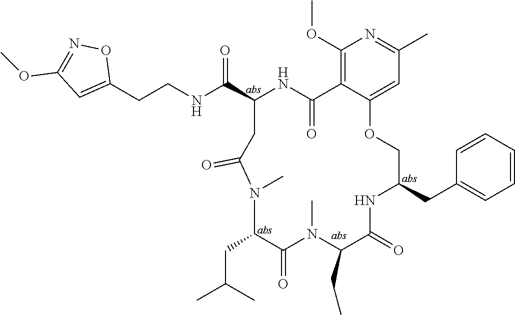 [SMILES: CC[C@@H]1C(=O)N[C@H](Cc2ccccc2)COc2cc(C)nc(OC)c2C(=O)N[C@H](C(=O)NCCc2cc(OC)no2)CC(=O)N(C)[C@@H](CC(C)C)C(=O)N1C] | 784 |
| 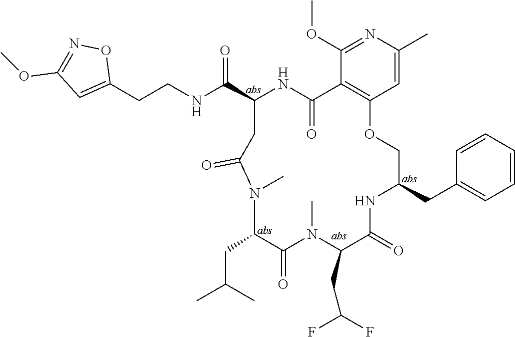 [SMILES: COc1cc(CCNC(=O)[C@@H]2CC(=O)N(C)[C@@H](CC(C)C)C(=O)N(C)[C@H](CC(F)F)C(=O)N[C@H](Cc3ccccc3)COc3cc(C)nc(OC)c3C(=O)N2)on1] | 785 |
| 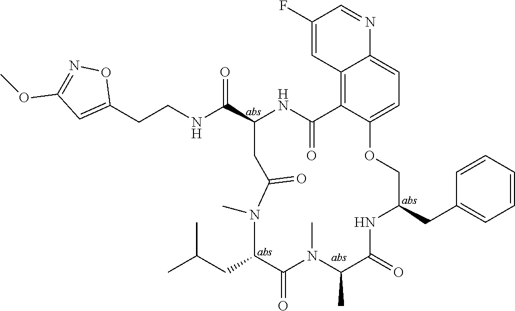 [SMILES: COc1cc(CCNC(=O)[C@@H]2CC(=O)N(C)[C@@H](CC(C)C)C(=O)N(C)[C@H](C)C(=O)N[C@H](Cc3ccccc3)COc3ccc4ncc(F)cc4c3C(=O)N2)on1] | 786 |
| 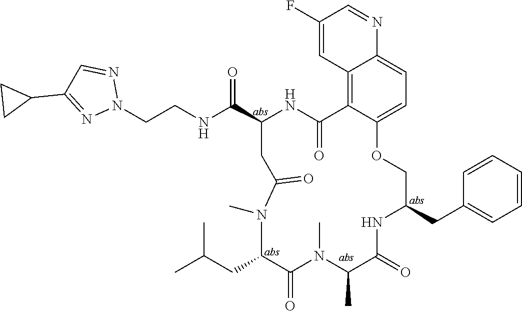 [SMILES: CC(C)C[C@H]1C(=O)N(C)[C@H](C)C(=O)N[C@H](Cc2ccccc2)COc2ccc3ncc(F)cc3c2C(=O)N[C@H](C(=O)NCCn2ncc(C3CC3)n2)CC(=O)N1C] | 787 |
| 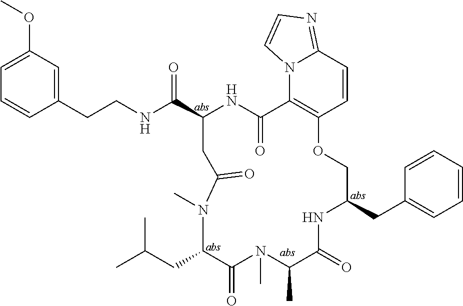 [SMILES: COc1cccc(CCNC(=O)[C@@H]2CC(=O)N(C)[C@@H](CC(C)C)C(=O)N(C)[C@H](C)C(=O)N[C@H](Cc3ccccc3)COc3ccc4nccn4c3C(=O)N2)c1] | 788 |
| 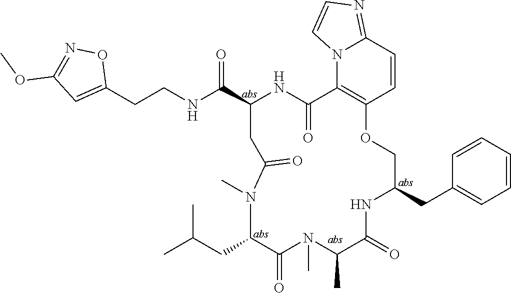 [SMILES: COc1cc(CCNC(=O)[C@@H]2CC(=O)N(C)[C@@H](CC(C)C)C(=O)N(C)[C@H](C)C(=O)N[C@H](Cc3ccccc3)COc3ccc4nccn4c3C(=O)N2)on1] | 789 |
| 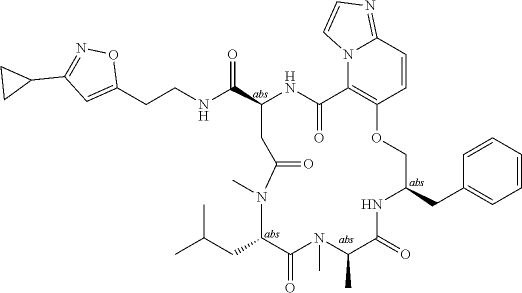 [SMILES: CC(C)C[C@H]1C(=O)N(C)[C@H](C)C(=O)N[C@H](Cc2ccccc2)COc2ccc3nccn3c2C(=O)N[C@H](C(=O)NCCc2cc(C3CC3)no2)CC(=O)N1C] | 790 |
| 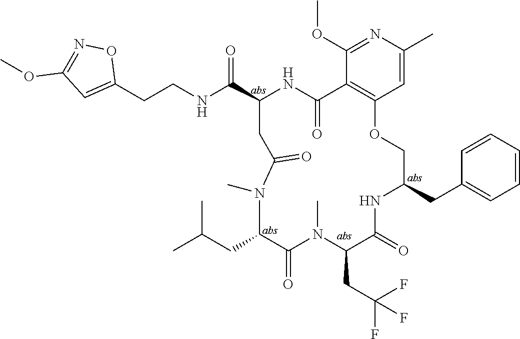 [SMILES: COc1cc(CCNC(=O)[C@@H]2CC(=O)N(C)[C@@H](CC(C)C)C(=O)N(C)[C@H](CC(F)(F)F)C(=O)N[C@H](Cc3ccccc3)COc3cc(C)nc(OC)c3C(=O)N2)on1] | 791 |
| 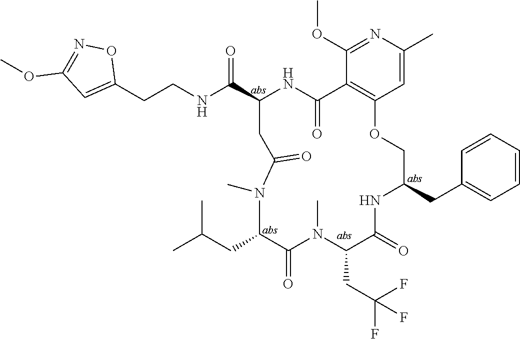 [SMILES: COc1cc(CCNC(=O)[C@@H]2CC(=O)N(C)[C@@H](CC(C)C)C(=O)N(C)[C@@H](CC(F)(F)F)C(=O)N[C@H](Cc3ccccc3)COc3cc(C)nc(OC)c3C(=O)N2)on1] | 792 |
| 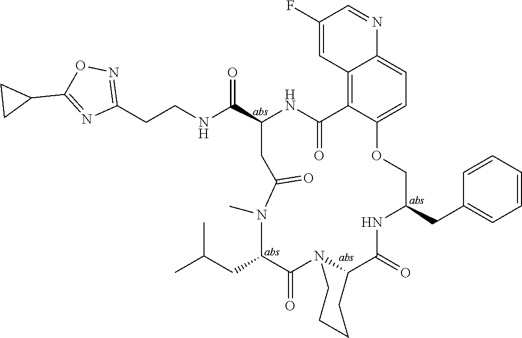 | 793 |
| 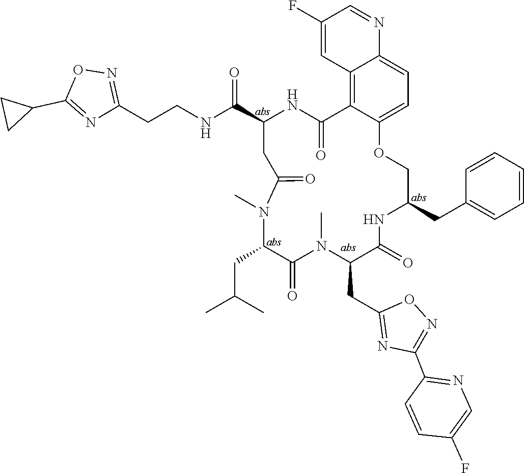 [SMILES: CC(C)C[C@H]1C(=O)N(C)[C@H](Cc2nc(-c3ccc(F)cn3)no2)C(=O)N[C@H](Cc2ccccc2)COc2ccc3ncc(F)cc3c2C(=O)N[C@H](C(=O)NCCc2noc(C3CC3)n2)CC(=O)N1C] | 794 |
| 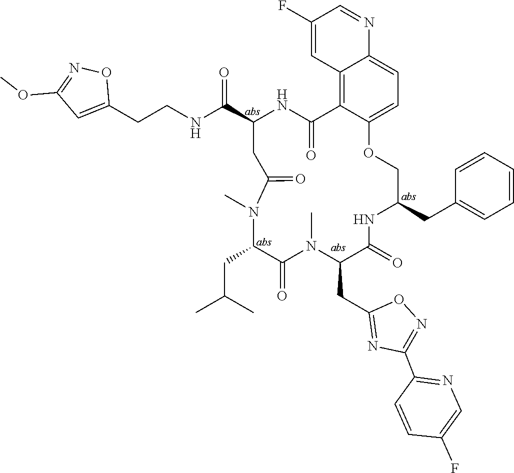 [SMILES: COc1cc(CCNC(=O)[C@@H]2CC(=O)N(C)[C@@H](CC(C)C)C(=O)N(C)[C@H](Cc3nc(-c4ccc(F)cn4)no3)C(=O)N[C@H](Cc3ccccc3)COc3ccc4ncc(F)cc4c3C(=O)N2)on1] | 795 |
| 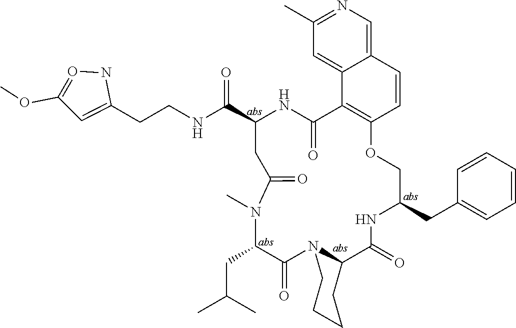 | 796 |
| 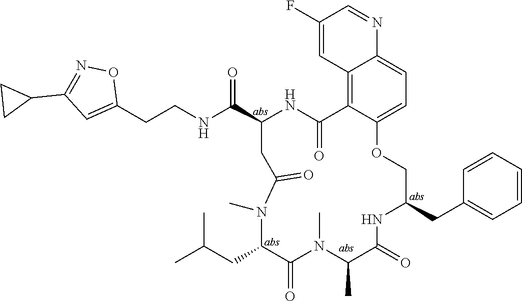 [SMILES: CC(C)C[C@H]1C(=O)N(C)[C@H](C)C(=O)N[C@H](Cc2ccccc2)COc2ccc3ncc(F)cc3c2C(=O)N[C@H](C(=O)NCCc2cc(C3CC3)no2)CC(=O)N1C] | 797 |
| 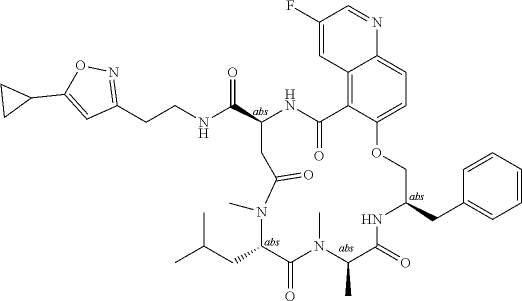 [SMILES: CC(C)C[C@H]1C(=O)N(C)[C@H](C)C(=O)N[C@H](Cc2ccccc2)COc2ccc3ncc(F)cc3c2C(=O)N[C@H](C(=O)NCCc2cc(C3CC3)on2)CC(=O)N1C] | 798 |
| 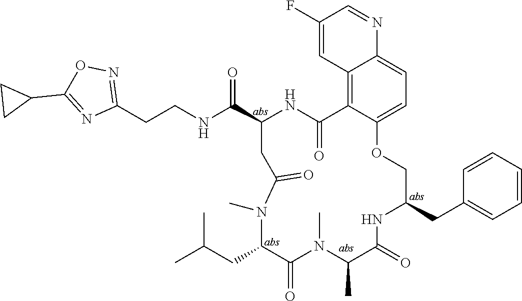 [SMILES: CC(C)C[C@H]1C(=O)N(C)[C@H](C)C(=O)N[C@H](Cc2ccccc2)COc2ccc3ncc(F)cc3c2C(=O)N[C@H](C(=O)NCCc2noc(C3CC3)n2)CC(=O)N1C] | 799 |
| 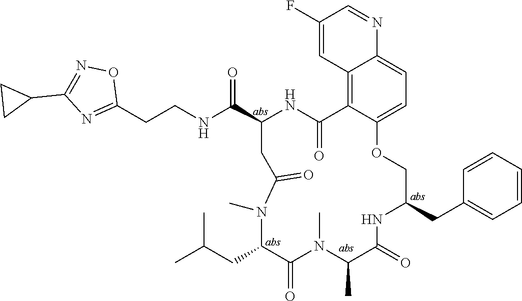 [SMILES: CC(C)C[C@H]1C(=O)N(C)[C@H](C)C(=O)N[C@H](Cc2ccccc2)COc2ccc3ncc(F)cc3c2C(=O)N[C@H](C(=O)NCCc2nc(C3CC3)no2)CC(=O)N1C] | 800 |
| 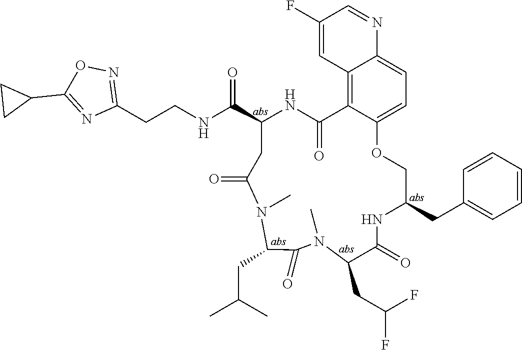 [SMILES: CC(C)C[C@H]1C(=O)N(C)[C@H](CC(F)F)C(=O)N[C@H](Cc2ccccc2)COc2ccc3ncc(F)cc3c2C(=O)N[C@H](C(=O)NCCc2noc(C3CC3)n2)CC(=O)N1C] | 801 |
| 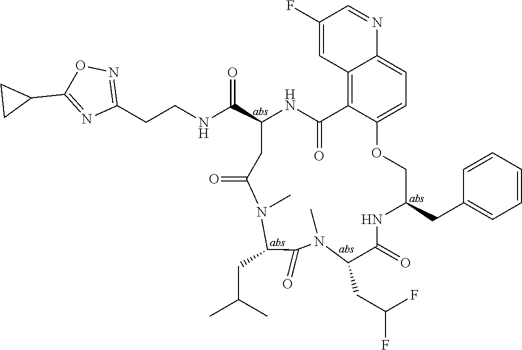 [SMILES: CC(C)C[C@H]1C(=O)N(C)[C@@H](CC(F)F)C(=O)N[C@H](Cc2ccccc2)COc2ccc3ncc(F)cc3c2C(=O)N[C@H](C(=O)NCCc2noc(C3CC3)n2)CC(=O)N1C] | 802 |
| 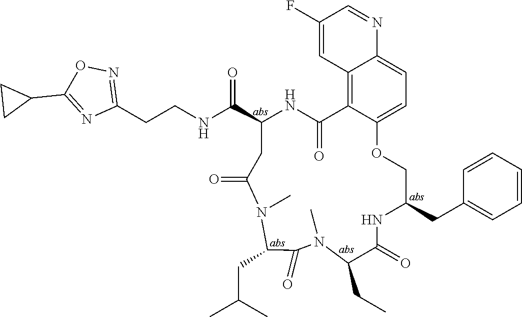 [SMILES: CC[C@@H]1C(=O)N[C@H](Cc2ccccc2)COc2ccc3ncc(F)cc3c2C(=O)N[C@H](C(=O)NCCc2noc(C3CC3)n2)CC(=O)N(C)[C@@H](CC(C)C)C(=O)N1C] | 803 |
| 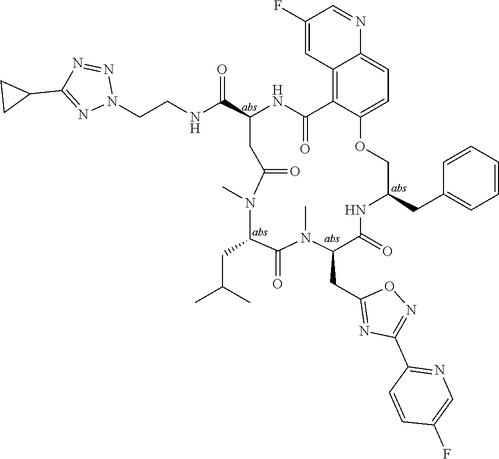 [SMILES: CC(C)C[C@H]1C(=O)N(C)[C@H](Cc2nc(-c3ccc(F)cn3)no2)C(=O)N[C@H](Cc2ccccc2)COc2ccc3ncc(F)cc3c2C(=O)N[C@H](C(=O)NCCn2nnc(C3CC3)n2)CC(=O)N1C] | 804 |
| 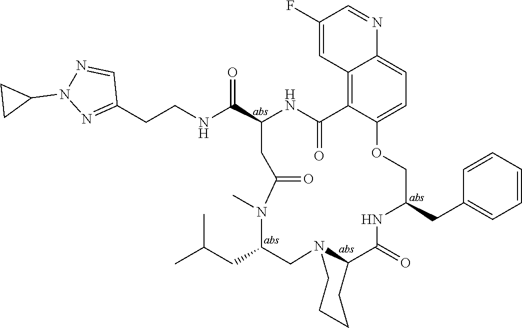 | 805 |
| 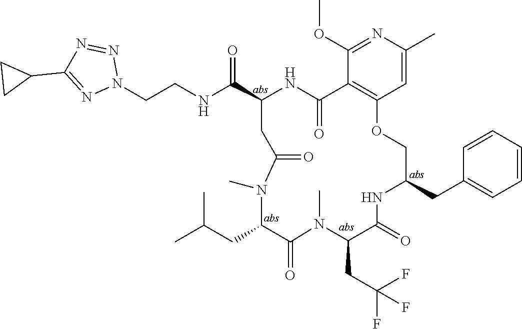 [SMILES: COc1nc(C)cc2c1C(=O)N[C@H](C(=O)NCCn1nnc(C3CC3)n1)CC(=O)N(C)[C@@H](CC(C)C)C(=O)N(C)[C@H](CC(F)(F)F)C(=O)N[C@H](Cc1ccccc1)CO2] | 806 |
| 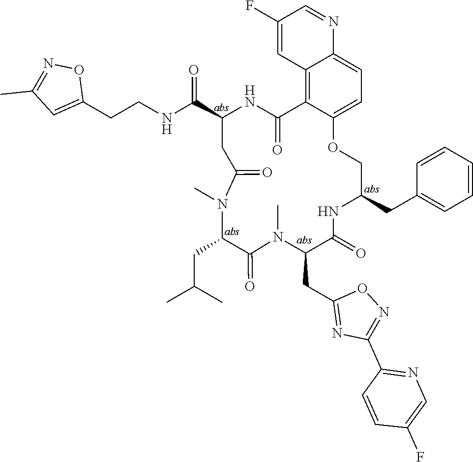 [SMILES: Cc1cc(CCNC(=O)[C@@H]2CC(=O)N(C)[C@@H](CC(C)C)C(=O)N(C)[C@H](Cc3nc(-c4ccc(F)cn4)no3)C(=O)N[C@H](Cc3ccccc3)COc3ccc4ncc(F)cc4c3C(=O)N2)on1] | 807 |
| 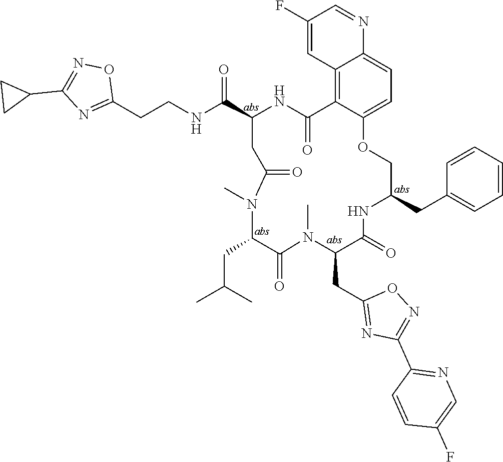 [SMILES: CC(C)C[C@H]1C(=O)N(C)[C@H](Cc2nc(-c3ccc(F)cn3)no2)C(=O)N[C@H](Cc2ccccc2)COc2ccc3ncc(F)cc3c2C(=O)N[C@H](C(=O)NCCc2nc(C3CC3)no2)CC(=O)N1C] | 808 |
| 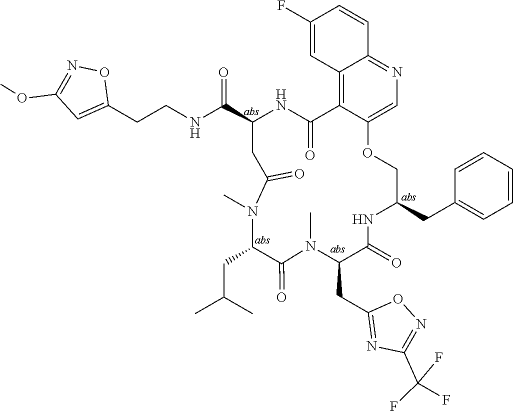 [SMILES: COc1cc(CCNC(=O)[C@@H]2CC(=O)N(C)[C@@H](CC(C)C)C(=O)N(C)[C@H](Cc3nc(C(F)(F)F)no3)C(=O)N[C@H](Cc3ccccc3)COc3cnc4ccc(F)cc4c3C(=O)N2)on1] | 809 |
| 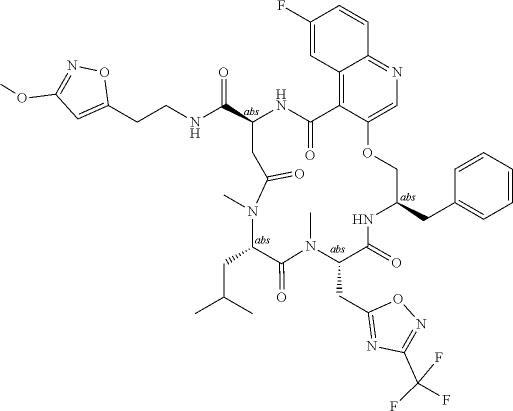 [SMILES: COc1cc(CCNC(=O)[C@@H]2CC(=O)N(C)[C@@H](CC(C)C)C(=O)N(C)[C@@H](Cc3nc(C(F)(F)F)no3)C(=O)N[C@H](Cc3ccccc3)COc3cnc4ccc(F)cc4c3C(=O)N2)on1] | 810 |
| 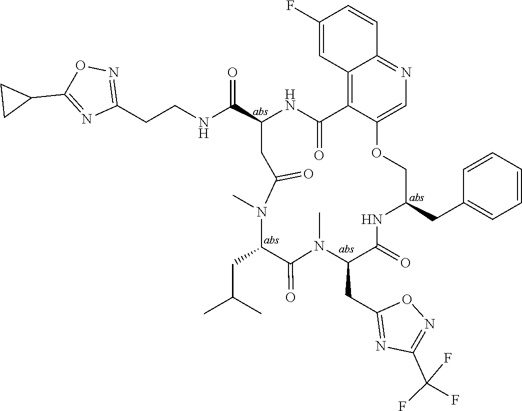 [SMILES: CC(C)C[C@H]1C(=O)N(C)[C@H](Cc2nc(C(F)(F)F)no2)C(=O)N[C@H](Cc2ccccc2)COc2cnc3ccc(F)cc3c2C(=O)N[C@H](C(=O)NCCc2noc(C3CC3)n2)CC(=O)N1C] | 811 |
| 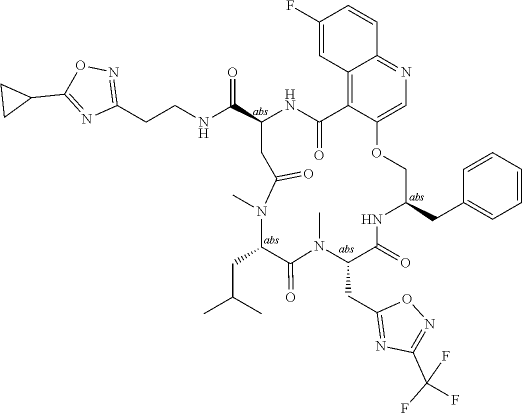 [SMILES: CC(C)C[C@H]1C(=O)N(C)[C@@H](Cc2nc(C(F)(F)F)no2)C(=O)N[C@H](Cc2ccccc2)COc2cnc3ccc(F)cc3c2C(=O)N[C@H](C(=O)NCCc2noc(C3CC3)n2)CC(=O)N1C] | 812 |
| 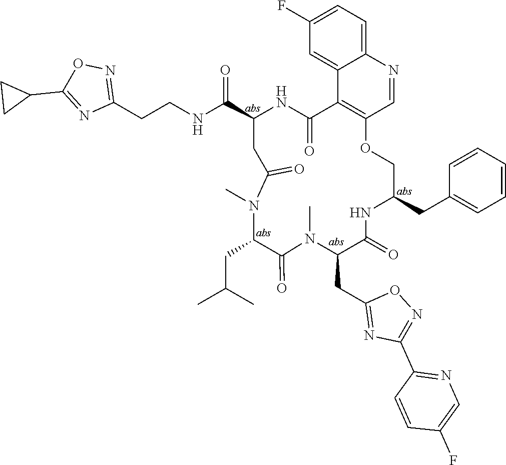 [SMILES: CC(C)C[C@H]1C(=O)N(C)[C@H](Cc2nc(-c3ccc(F)cn3)no2)C(=O)N[C@H](Cc2ccccc2)COc2cnc3ccc(F)cc3c2C(=O)N[C@H](C(=O)NCCc2noc(C3CC3)n2)CC(=O)N1C] | 813 |
| 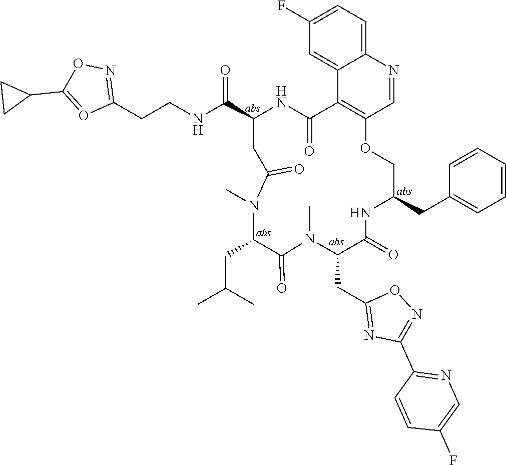 | 814 |
| 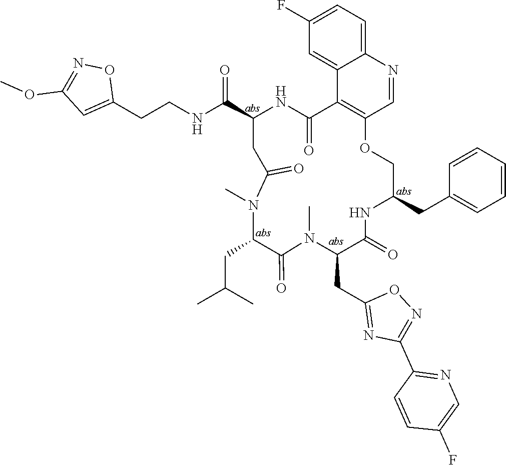 [SMILES: COc1cc(CCNC(=O)[C@@H]2CC(=O)N(C)[C@@H](CC(C)C)C(=O)N(C)[C@H](Cc3nc(-c4ccc(F)cn4)no3)C(=O)N[C@H](Cc3ccccc3)COc3cnc4ccc(F)cc4c3C(=O)N2)on1] | 815 |
| 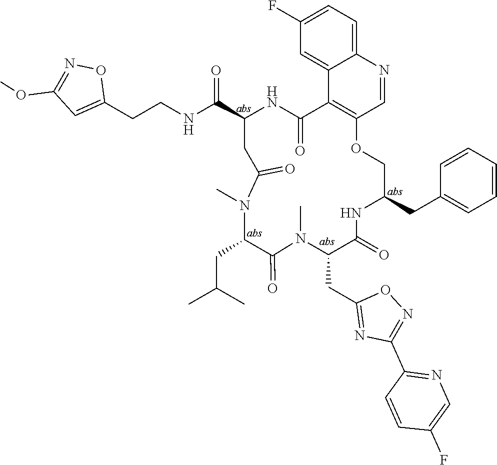 [SMILES: COc1cc(CCNC(=O)[C@@H]2CC(=O)N(C)[C@@H](CC(C)C)C(=O)N(C)[C@@H](Cc3nc(-c4ccc(F)cn4)no3)C(=O)N[C@H](Cc3ccccc3)COc3cnc4ccc(F)cc4c3C(=O)N2)on1] | 816 |
| 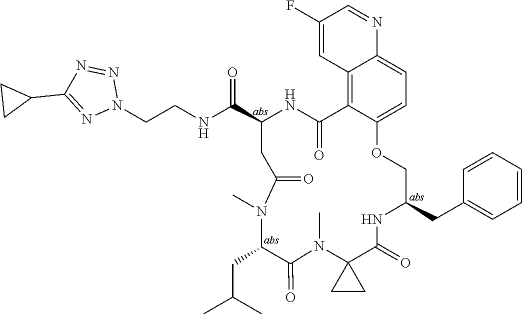 [SMILES: CC(C)C[C@H]1C(=O)N(C)C2(CC2)C(=O)N[C@H](Cc2ccccc2)COc2ccc3ncc(F)cc3c2C(=O)N[C@H](C(=O)NCCn2nnc(C3CC3)n2)CC(=O)N1C] | 817 |
| 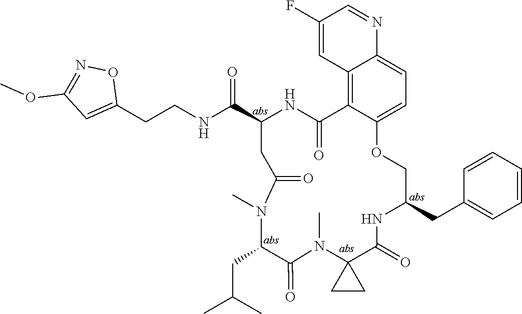 [SMILES: COc1cc(CCNC(=O)[C@@H]2CC(=O)N(C)[C@@H](CC(C)C)C(=O)N(C)C3(CC3)C(=O)N[C@H](Cc3ccccc3)COc3ccc4ncc(F)cc4c3C(=O)N2)on1] | 818 |
| 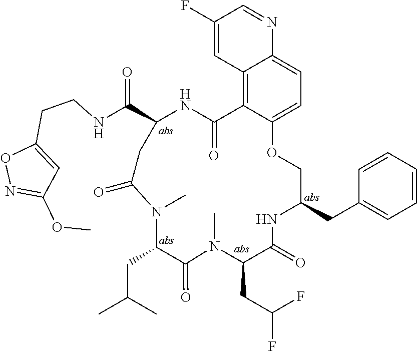 [SMILES: COc1cc(CCNC(=O)[C@@H]2CC(=O)N(C)[C@@H](CC(C)C)C(=O)N(C)[C@H](CC(F)F)C(=O)N[C@H](Cc3ccccc3)COc3ccc4ncc(F)cc4c3C(=O)N2)on1] | 819 |
| 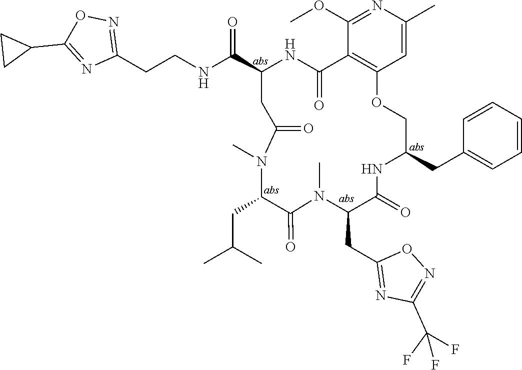 [SMILES: COc1nc(C)cc2c1C(=O)N[C@H](C(=O)NCCc1noc(C3CC3)n1)CC(=O)N(C)[C@@H](CC(C)C)C(=O)N(C)[C@H](Cc1nc(C(F)(F)F)no1)C(=O)N[C@H](Cc1ccccc1)CO2] | 820 |
| 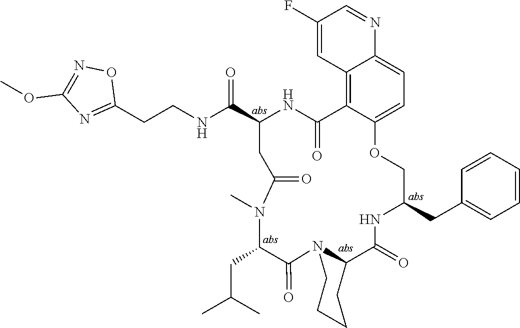 | 821 |
| 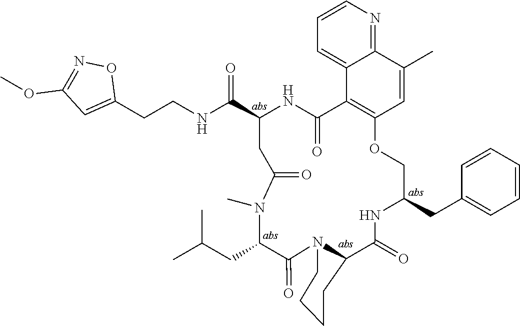 | 822 |
| 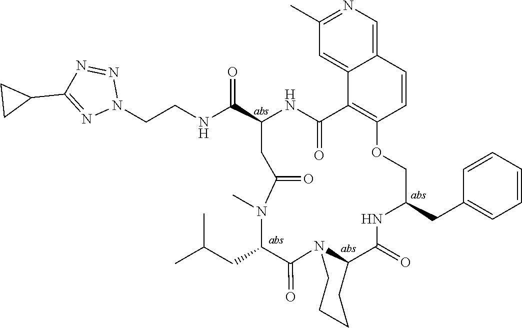 | 823 |
| 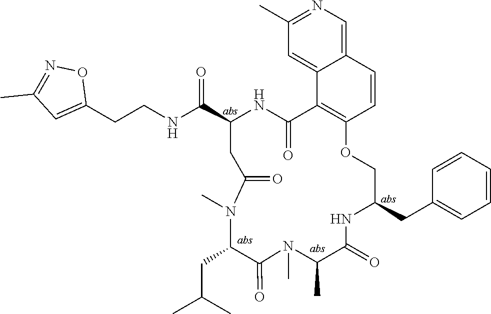 [SMILES: Cc1cc2c3c(ccc2cn1)OC[C@@H](Cc1ccccc1)NC(=O)[C@@H](C)N(C)C(=O)[C@H](CC(C)C)N(C)C(=O)C[C@@H](C(=O)NCCc1cc(C)no1)NC3=O] | 824 |
| 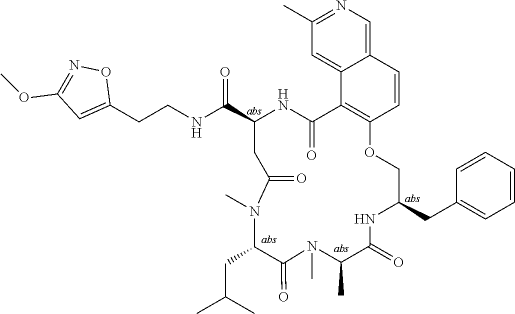 [SMILES: COc1cc(CCNC(=O)[C@@H]2CC(=O)N(C)[C@@H](CC(C)C)C(=O)N(C)[C@H](C)C(=O)N[C@H](Cc3ccccc3)COc3ccc4cnc(C)cc4c3C(=O)N2)on1] | 825 |
| 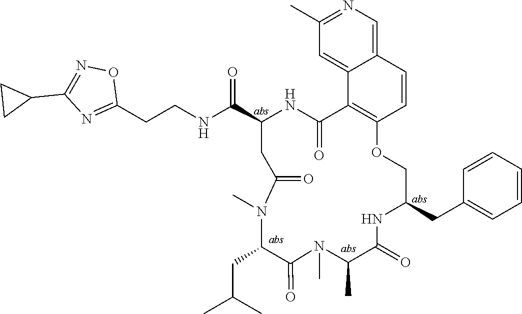 [SMILES: Cc1cc2c3c(ccc2cn1)OC[C@@H](Cc1ccccc1)NC(=O)[C@@H](C)N(C)C(=O)[C@H](CC(C)C)N(C)C(=O)C[C@@H](C(=O)NCCc1nc(C2CC2)no1)NC3=O] | 826 |
| 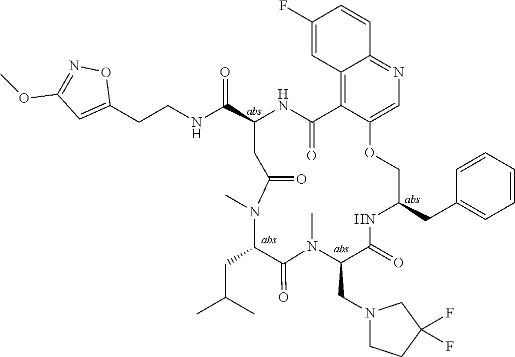 [SMILES: COc1cc(CCNC(=O)[C@@H]2CC(=O)N(C)[C@@H](CC(C)C)C(=O)N(C)[C@H](CN3CCC(F)(F)C3)C(=O)N[C@H](Cc3ccccc3)COc3cnc4ccc(F)cc4c3C(=O)N2)on1] | 827 |
| 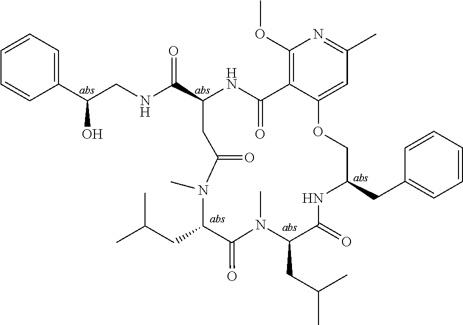 [SMILES: COc1nc(C)cc2c1C(=O)N[C@H](C(=O)NC[C@@H](O)c1ccccc1)CC(=O)N(C)[C@@H](CC(C)C)C(=O)N(C)[C@H](CC(C)C)C(=O)N[C@H](Cc1ccccc1)CO2] | 828 |
| 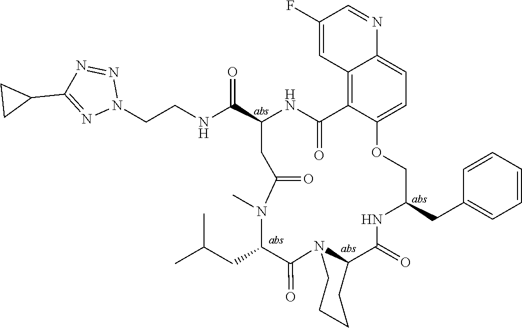 | 829 |

TABLE 5
Structures of compounds of Example 830 to 983

| | |
|---|---|
| 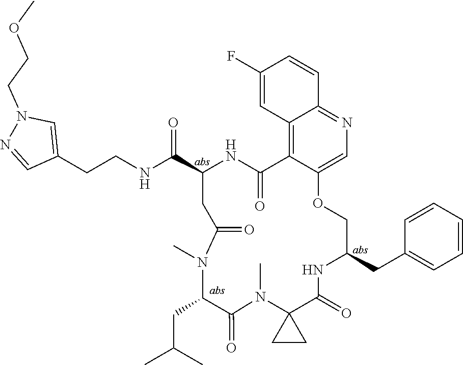 [SMILES: COCCn1cc(CCNC(=O)[C@@H]2CC(=O)N(C)[C@@H](CC(C)C)C(=O)N(C)C3(CC3)C(=O)N[C@H](Cc3ccccc3)COc3cnc4ccc(F)cc4c3C(=O)N2)cn1] | 830 |
| 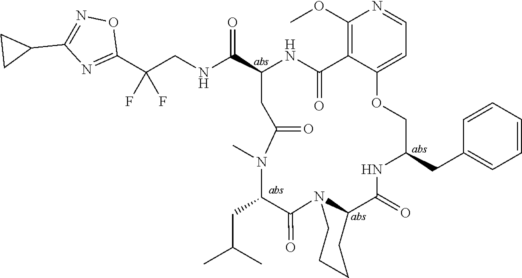 | 831 |
| 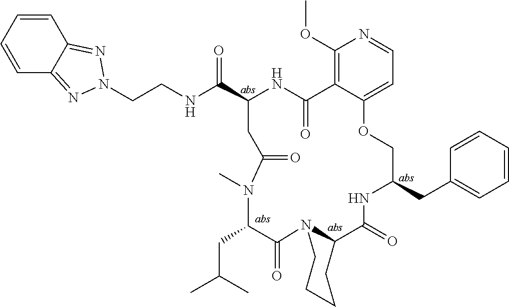 | 832 |
| 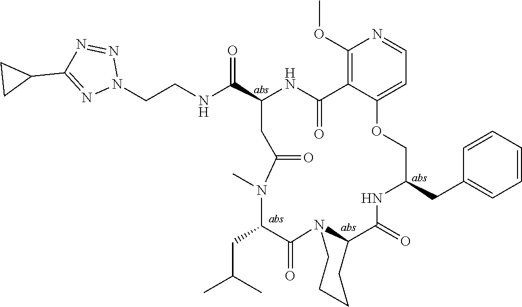 | 833 |
| 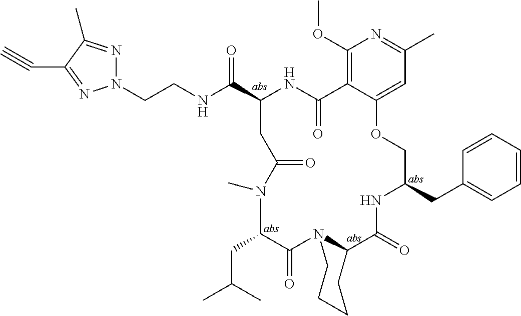 | 834 |
| 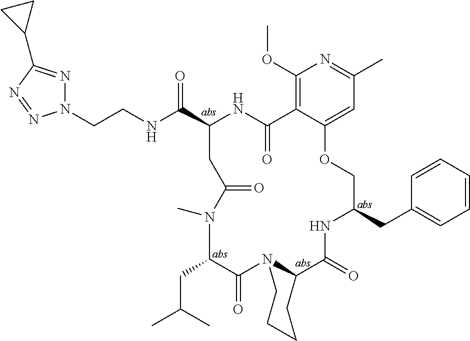 | 835 |
| 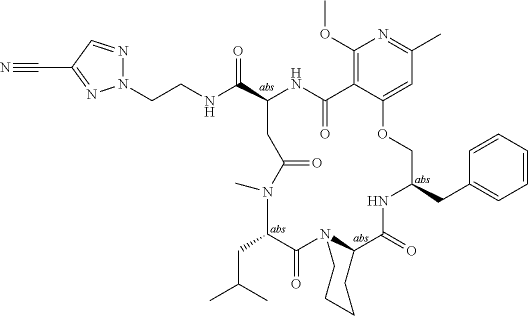 | 836 |
| 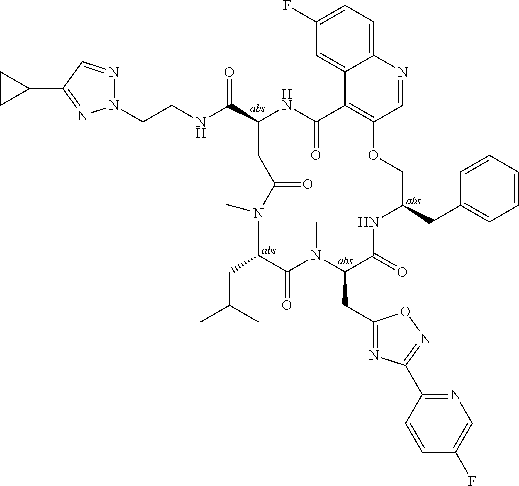 [SMILES: CC(C)C[C@H]1C(=O)N(C)[C@H](Cc2nc(-c3ccc(F)cn3)no2)C(=O)N[C@H](Cc2ccccc2)COc2cnc3ccc(F)cc3c2C(=O)N[C@H](C(=O)NCCn2ncc(C3CC3)n2)CC(=O)N1C] | 837 |
| 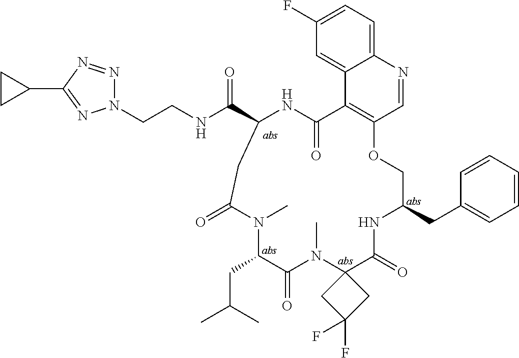 [SMILES: CC(C)C[C@H]1C(=O)N(C)C2(CC(F)(F)C2)C(=O)N[C@H](Cc2ccccc2)COc2cnc3ccc(F)cc3c2C(=O)N[C@H](C(=O)NCCn2nnc(C3CC3)n2)CC(=O)N1C] | 838 |
| 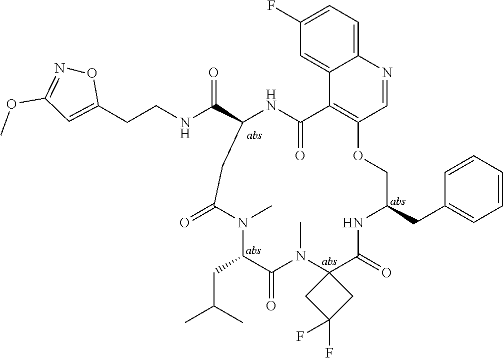 [SMILES: COc1cc(CCNC(=O)[C@@H]2CC(=O)N(C)[C@@H](CC(C)C)C(=O)N(C)C3(CC(F)(F)C3)C(=O)N[C@H](Cc3ccccc3)COc3cnc4ccc(F)cc4c3C(=O)N2)on1] | 839 |
| 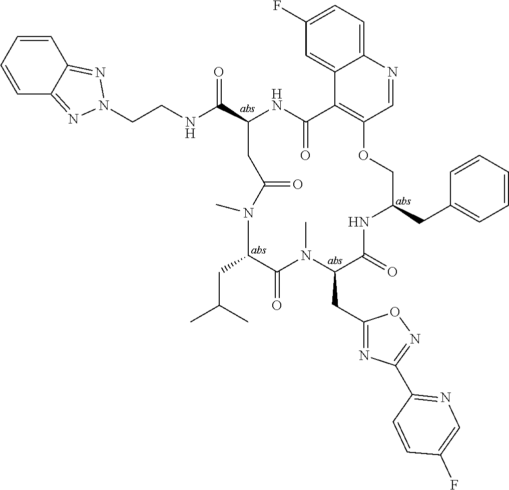 [SMILES: CC(C)C[C@H]1C(=O)N(C)[C@H](Cc2nc(-c3ccc(F)cn3)no2)C(=O)N[C@H](Cc2ccccc2)COc2cnc3ccc(F)cc3c2C(=O)N[C@H](C(=O)NCCn2nc3ccccc3n2)CC(=O)N1C] | 840 |
| 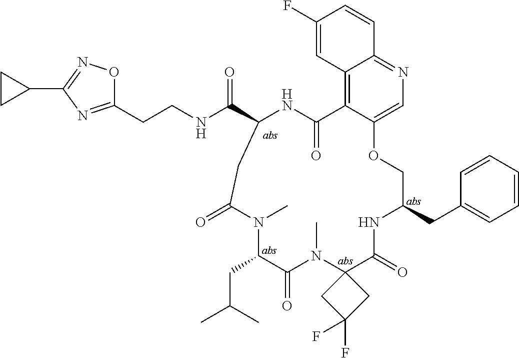 [SMILES: CC(C)C[C@H]1C(=O)N(C)C2(CC(F)(F)C2)C(=O)N[C@H](Cc2ccccc2)COc2cnc3ccc(F)cc3c2C(=O)N[C@H](C(=O)NCCc2nc(C3CC3)no2)CC(=O)N1C] | 841 |
| 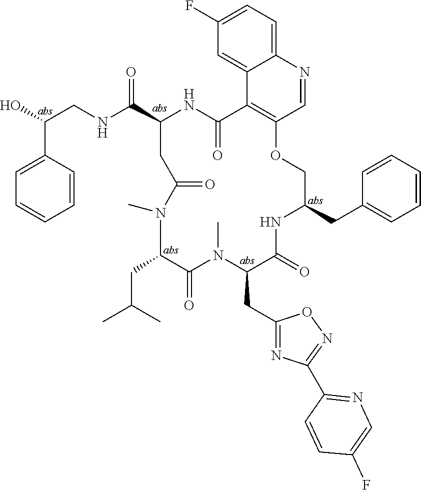 [SMILES: CC(C)C[C@H]1C(=O)N(C)[C@H](Cc2nc(-c3ccc(F)cn3)no2)C(=O)N[C@H](Cc2ccccc2)COc2cnc3ccc(F)cc3c2C(=O)N[C@H](C(=O)NC[C@@H](O)c2ccccc2)CC(=O)N1C] | 842 |
| 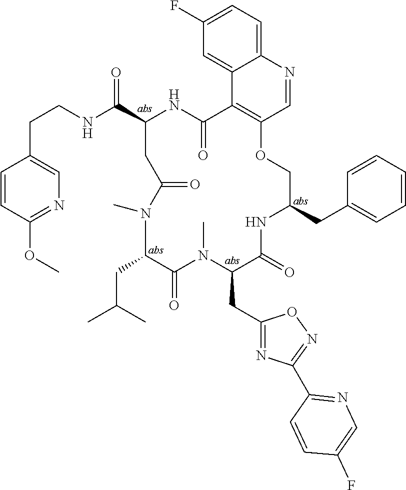 [SMILES: COc1ccc(CCNC(=O)[C@@H]2CC(=O)N(C)[C@@H](CC(C)C)C(=O)N(C)[C@H](Cc3nc(-c4ccc(F)cn4)no3)C(=O)N[C@H](Cc3ccccc3)COc3cnc4ccc(F)cc4c3C(=O)N2)cn1] | 843 |
| 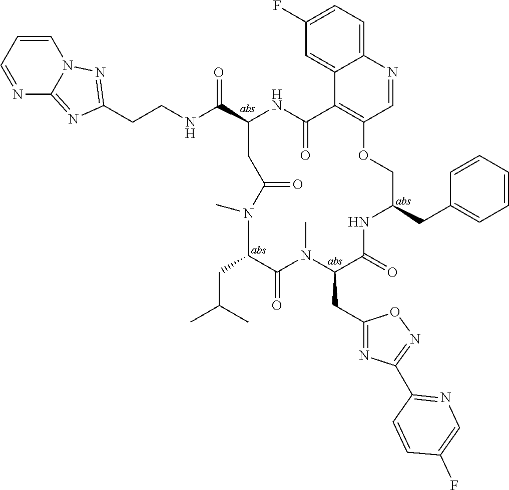 [SMILES: CC(C)C[C@H]1C(=O)N(C)[C@H](Cc2nc(-c3ccc(F)cn3)no2)C(=O)N[C@H](Cc2ccccc2)COc2cnc3ccc(F)cc3c2C(=O)N[C@H](C(=O)NCCc2nc3ncccn3n2)CC(=O)N1C] | 844 |
| 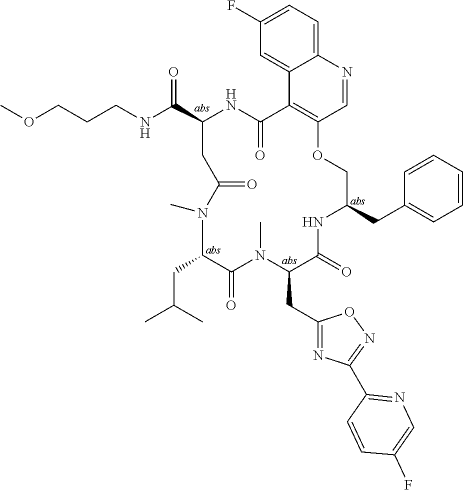 [SMILES: COCCCNC(=O)[C@@H]1CC(=O)N(C)[C@@H](CC(C)C)C(=O)N(C)[C@H](Cc2nc(-c3ccc(F)cn3)no2)C(=O)N[C@H](Cc2ccccc2)COc2cnc3ccc(F)cc3c2C(=O)N1] | 845 |
| 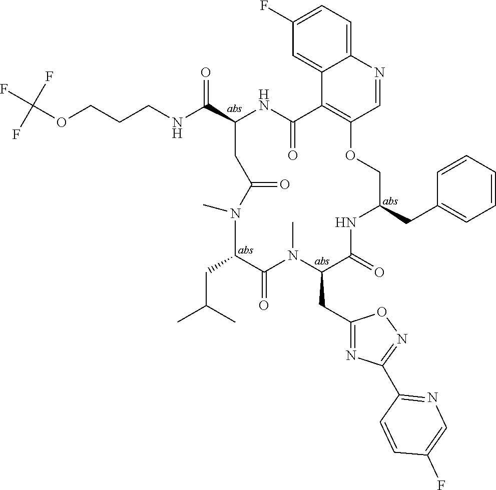 [SMILES: CC(C)C[C@H]1C(=O)N(C)[C@H](Cc2nc(-c3ccc(F)cn3)no2)C(=O)N[C@H](Cc2ccccc2)COc2cnc3ccc(F)cc3c2C(=O)N[C@H](C(=O)NCCCOC(F)(F)F)CC(=O)N1C] | 846 |
| 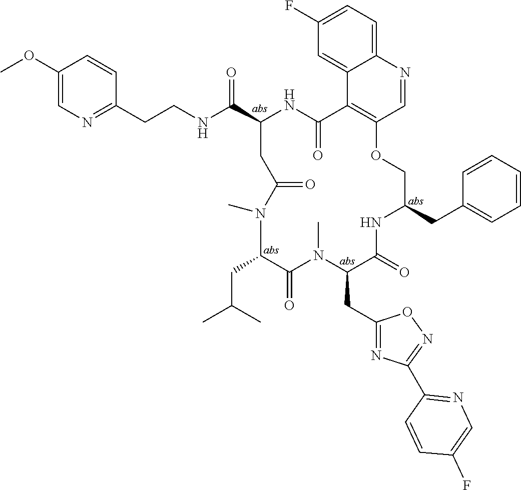 [SMILES: COc1ccc(CCNC(=O)[C@@H]2CC(=O)N(C)[C@@H](CC(C)C)C(=O)N(C)[C@H](Cc3nc(-c4ccc(F)cn4)no3)C(=O)N[C@H](Cc3ccccc3)COc3cnc4ccc(F)cc4c3C(=O)N2)nc1] | 847 |
| 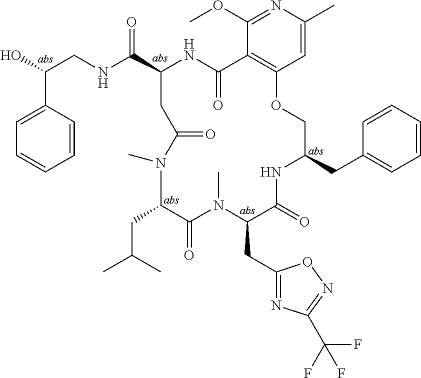 [SMILES: COc1nc(C)cc2c1C(=O)N[C@H](C(=O)NC[C@@H](O)c1ccccc1)CC(=O)N(C)[C@@H](CC(C)C)C(=O)N(C)[C@H](Cc1nc(C(F)(F)F)no1)C(=O)N[C@H](Cc1ccccc1)CO2] | 848 |
| 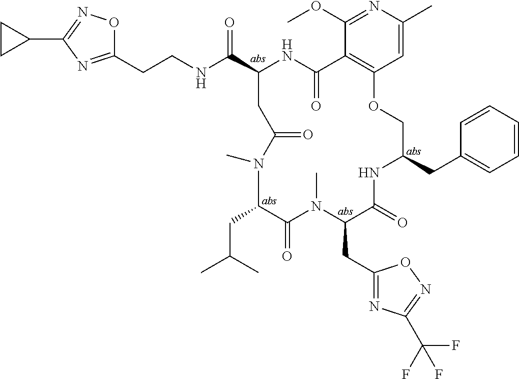 [SMILES: COc1nc(C)cc2c1C(=O)N[C@H](C(=O)NCCc1nc(C3CC3)no1)CC(=O)N(C)[C@@H](CC(C)C)C(=O)N(C)[C@H](Cc1nc(C(F)(F)F)no1)C(=O)N[C@H](Cc1ccccc1)CO2] | 849 |
| 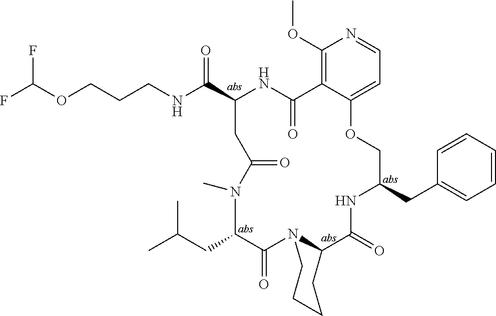 | 850 |
| 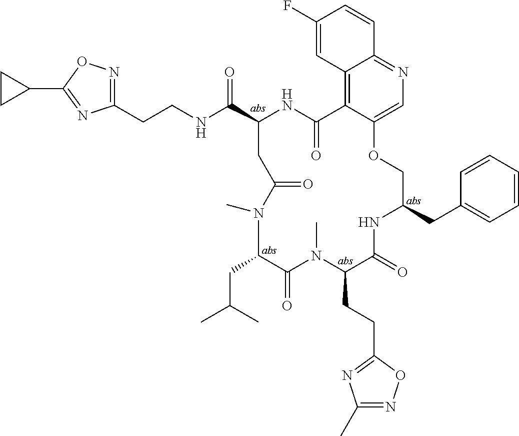 [SMILES: Cc1noc(CC[C@@H]2C(=O)N[C@H](Cc3ccccc3)COc3cnc4ccc(F)cc4c3C(=O)N[C@H](C(=O)NCCc3noc(C4CC4)n3)CC(=O)N(C)[C@@H](CC(C)C)C(=O)N2C)n1] | 851 |
| 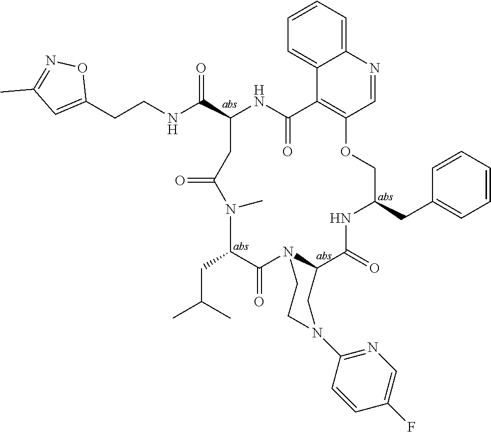 | 852 |
| 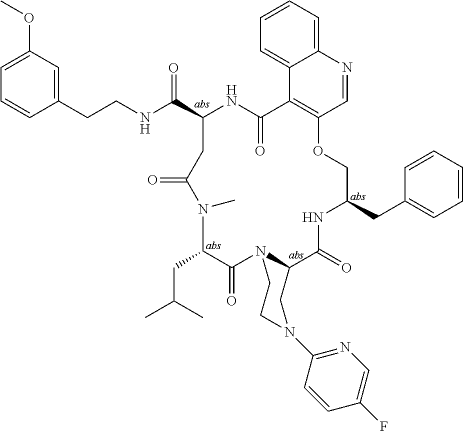 | 853 |
| 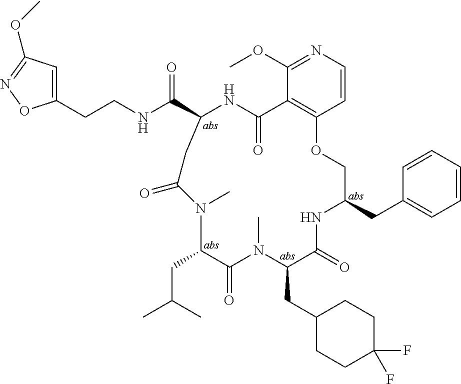 [SMILES: COc1cc(CCNC(=O)[C@@H]2CC(=O)N(C)[C@@H](CC(C)C)C(=O)N(C)[C@H](CC3CCC(F)(F)CC3)C(=O)N[C@H](Cc3ccccc3)COc3ccnc(OC)c3C(=O)N2)on1] | 854 |
| 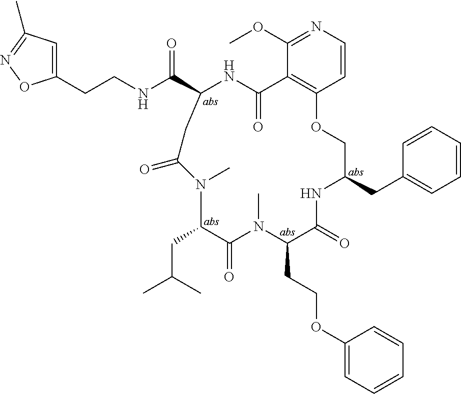 [SMILES: COc1nccc2c1C(=O)N[C@H](C(=O)NCCc1cc(C)no1)CC(=O)N(C)[C@@H](CC(C)C)C(=O)N(C)[C@H](CCOc1ccccc1)C(=O)N[C@H](Cc1ccccc1)CO2] | 855 |
| 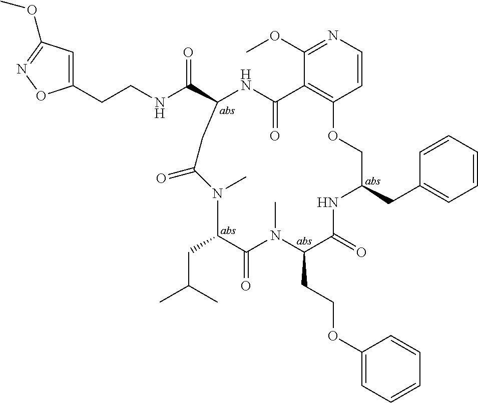 [SMILES: COc1cc(CCNC(=O)[C@@H]2CC(=O)N(C)[C@@H](CC(C)C)C(=O)N(C)[C@H](CCOc3ccccc3)C(=O)N[C@H](Cc3ccccc3)COc3ccnc(OC)c3C(=O)N2)on1] | 856 |
| 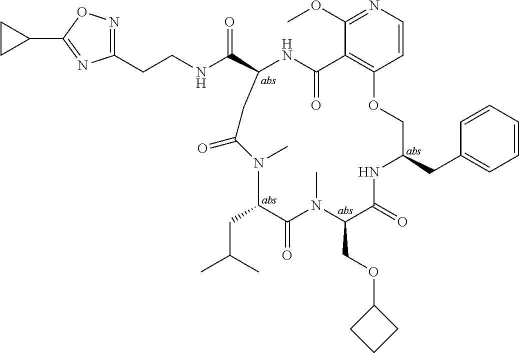 [SMILES: COc1nccc2c1C(=O)N[C@H](C(=O)NCCc1noc(C3CC3)n1)CC(=O)N(C)[C@@H](CC(C)C)C(=O)N(C)[C@H](COC1CCC1)C(=O)N[C@H](Cc1ccccc1)CO2] | 857 |
| 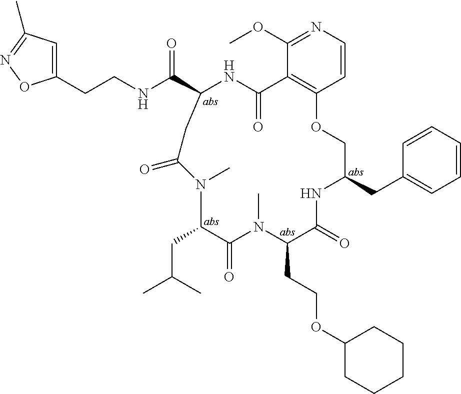 [SMILES: COc1nccc2c1C(=O)N[C@H](C(=O)NCCc1cc(C)no1)CC(=O)N(C)[C@@H](CC(C)C)C(=O)N(C)[C@H](CCOC1CCCCC1)C(=O)N[C@H](Cc1ccccc1)CO2] | 858 |
| 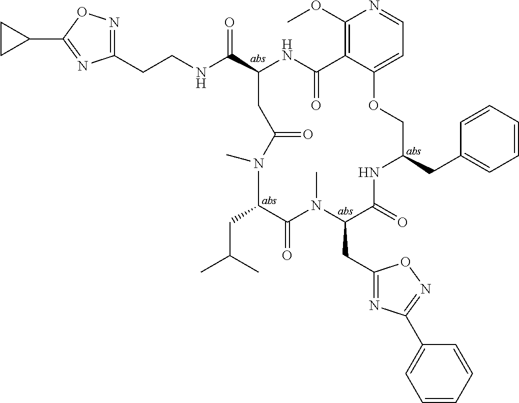 [SMILES: COc1nccc2c1C(=O)N[C@H](C(=O)NCCc1noc(C3CC3)n1)CC(=O)N(C)[C@@H](CC(C)C)C(=O)N(C)[C@H](Cc1nc(-c3ccccc3)no1)C(=O)N[C@H](Cc1ccccc1)CO2] | 859 |
| 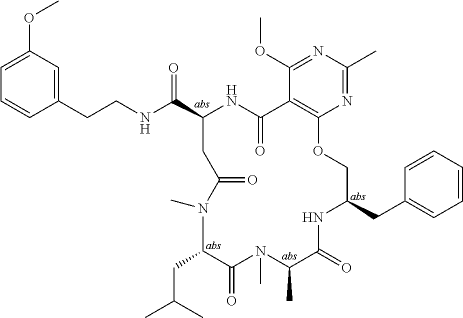 [SMILES: COc1cccc(CCNC(=O)[C@@H]2CC(=O)N(C)[C@@H](CC(C)C)C(=O)N(C)[C@H](C)C(=O)N[C@H](Cc3ccccc3)COc3nc(C)nc(OC)c3C(=O)N2)c1] | 860 |
| 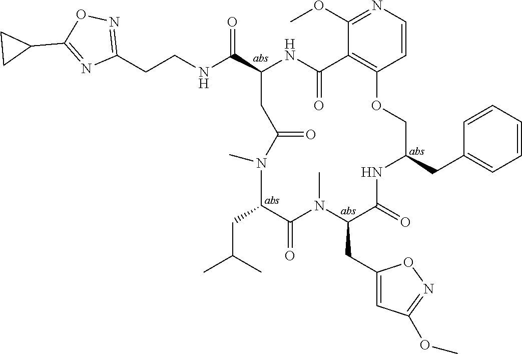 [SMILES: COc1cc(C[C@@H]2C(=O)N[C@H](Cc3ccccc3)COc3ccnc(OC)c3C(=O)N[C@H](C(=O)NCCc3noc(C4CC4)n3)CC(=O)N(C)[C@@H](CC(C)C)C(=O)N2C)on1] | 861 |
| 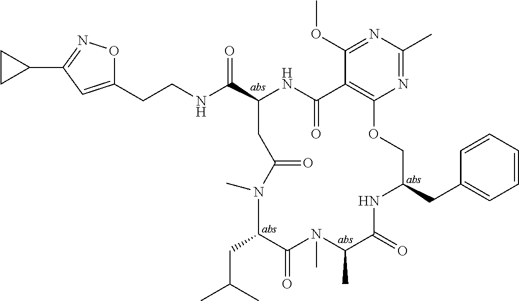 [SMILES: COc1nc(C)nc2c1C(=O)N[C@H](C(=O)NCCc1cc(C3CC3)no1)CC(=O)N(C)[C@@H](CC(C)C)C(=O)N(C)[C@H](C)C(=O)N[C@H](Cc1ccccc1)CO2] | 862 |
| 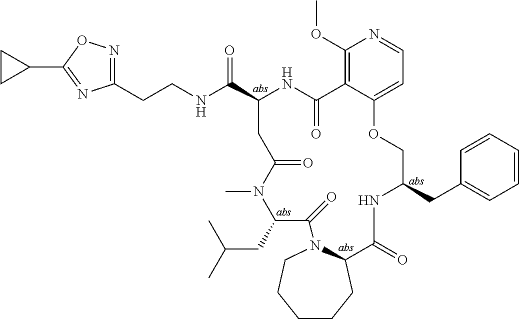 [SMILES: COc1nccc2c1C(=O)N[C@H](C(=O)NCCc1noc(C3CC3)n1)CC(=O)N(C)[C@@H](CC(C)C)C(=O)N1CCCCC[C@@H]1C(=O)N[C@H](Cc1ccccc1)CO2] | 863 |
| 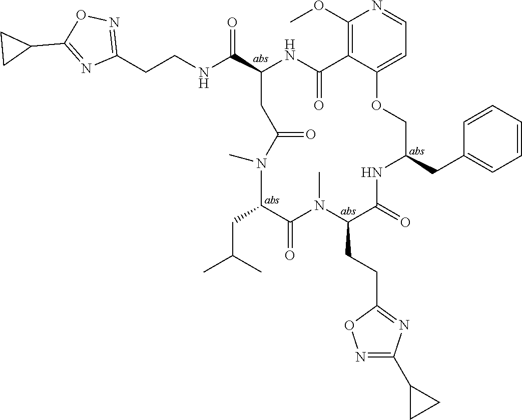 [SMILES: COc1nccc2c1C(=O)N[C@H](C(=O)NCCc1noc(C3CC3)n1)CC(=O)N(C)[C@@H](CC(C)C)C(=O)N(C)[C@H](CCc1nc(C3CC3)no1)C(=O)N[C@H](Cc1ccccc1)CO2] | 864 |
| 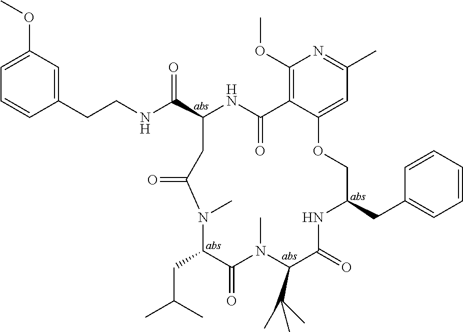 [SMILES: COc1cccc(CCNC(=O)[C@@H]2CC(=O)N(C)[C@@H](CC(C)C)C(=O)N(C)[C@H](C(C)(C)C)C(=O)N[C@H](Cc3ccccc3)COc3cc(C)nc(OC)c3C(=O)N2)c1] | 865 |
| 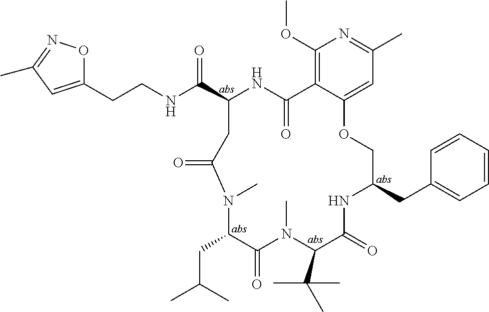 [SMILES: COc1nc(C)cc2c1C(=O)N[C@H](C(=O)NCCc1cc(C)no1)CC(=O)N(C)[C@@H](CC(C)C)C(=O)N(C)[C@H](C(C)(C)C)C(=O)N[C@H](Cc1ccccc1)CO2] | 866 |
| 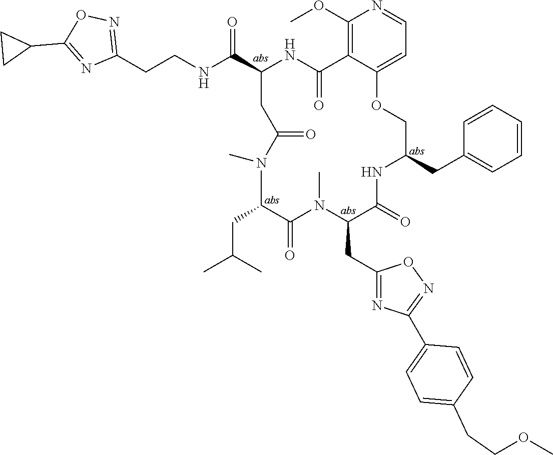 [SMILES: COCCc1ccc(-c2noc(C[C@@H]3C(=O)N[C@H](Cc4ccccc4)COc4ccnc(OC)c4C(=O)N[C@H](C(=O)NCCc4noc(C5CC5)n4)CC(=O)N(C)[C@@H](CC(C)C)C(=O)N3C)n2)cc1] | 867 |
| 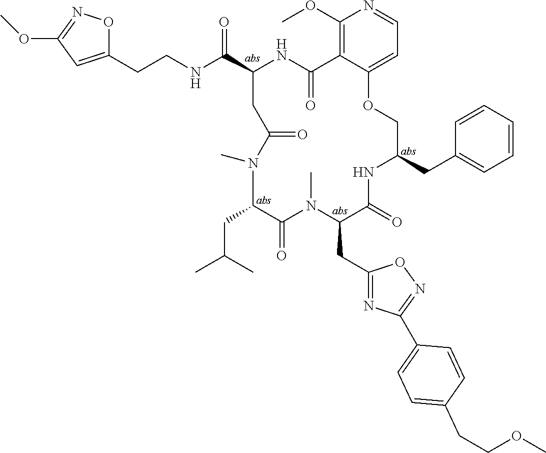 | 868 |
| 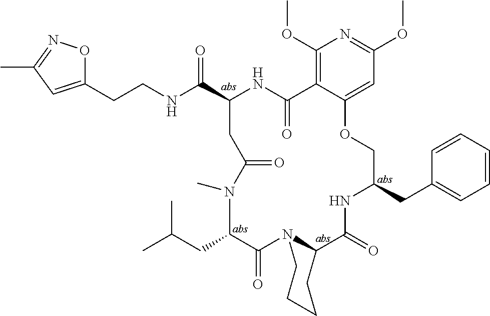 | 869 |
| 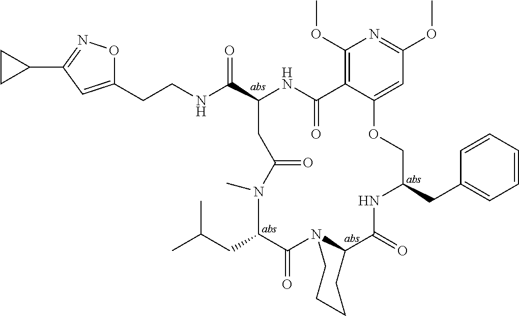 | 870 |
| 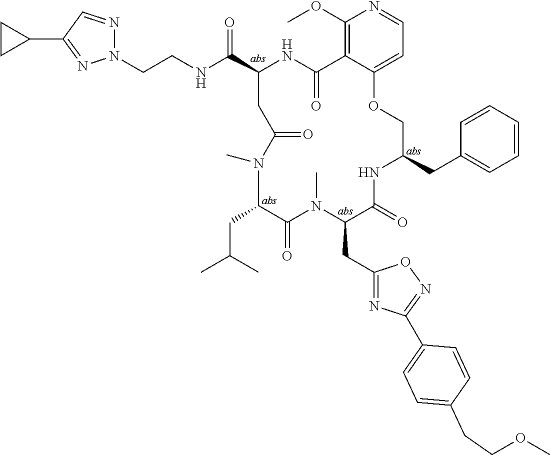 [SMILES: COCCc1ccc(-c2noc(C[C@@H]3C(=O)N[C@H](Cc4ccccc4)COc4ccnc(OC)c4C(=O)N[C@H](C(=O)NCCn4ncc(C5CC5)n4)CC(=O)N(C)[C@@H](CC(C)C)C(=O)N3C)n2)cc1] | 871 |
| 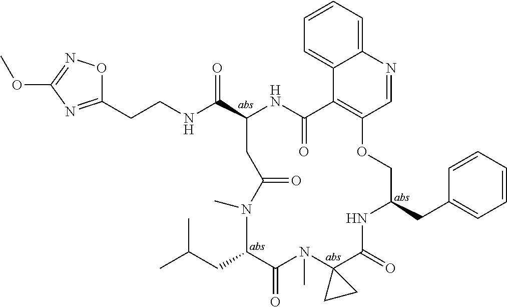 [SMILES: COc1noc(CCNC(=O)[C@@H]2CC(=O)N(C)[C@@H](CC(C)C)C(=O)N(C)C3(CC3)C(=O)N[C@H](Cc3ccccc3)COc3cnc4ccccc4c3C(=O)N2)n1] | 872 |
| 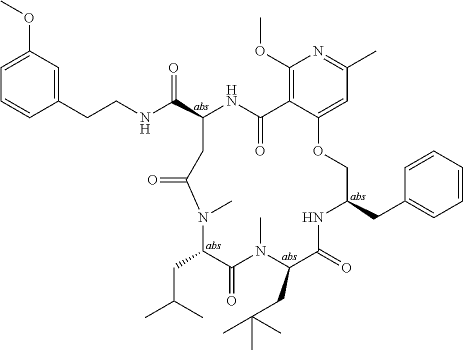 [SMILES: COc1cccc(CCNC(=O)[C@@H]2CC(=O)N(C)[C@@H](CC(C)C)C(=O)N(C)[C@H](CC(C)(C)C)C(=O)N[C@H](Cc3ccccc3)COc3cc(C)nc(OC)c3C(=O)N2)c1] | 873 |
| 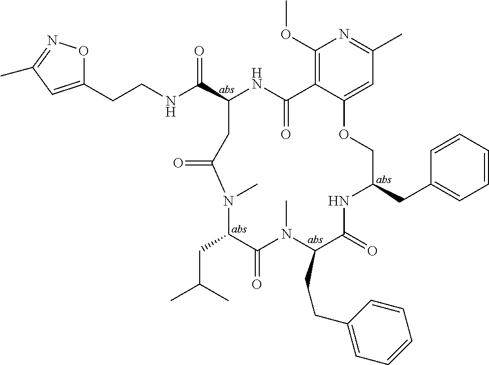 [SMILES: COc1nc(C)cc2c1C(=O)N[C@H](C(=O)NCCc1cc(C)no1)CC(=O)N(C)[C@@H](CC(C)C)C(=O)N(C)[C@H](CCc1ccccc1)C(=O)N[C@H](Cc1ccccc1)CO2] | 874 |
| 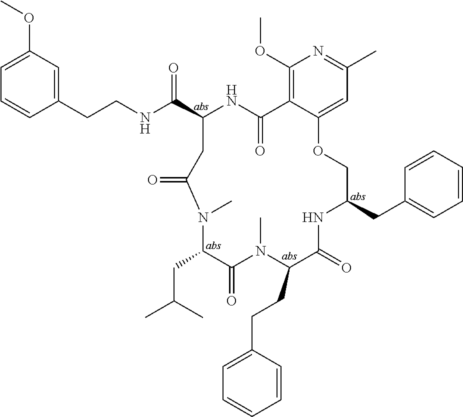 [SMILES: COc1cccc(CCNC(=O)[C@@H]2CC(=O)N(C)[C@@H](CC(C)C)C(=O)N(C)[C@H](CCc3ccccc3)C(=O)N[C@H](Cc3ccccc3)COc3cc(C)nc(OC)c3C(=O)N2)c1] | 875 |
| 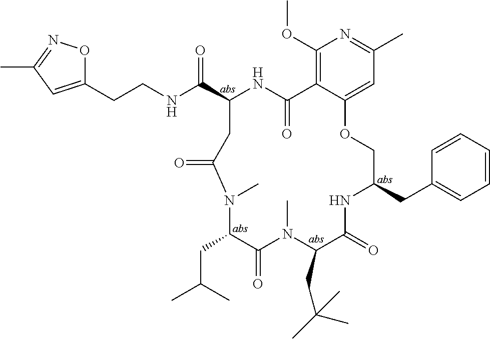 [SMILES: COc1nc(C)cc2c1C(=O)N[C@H](C(=O)NCCc1cc(C)no1)CC(=O)N(C)[C@@H](CC(C)C)C(=O)N(C)[C@H](CC(C)(C)C)C(=O)N[C@H](Cc1ccccc1)CO2] | 876 |
| 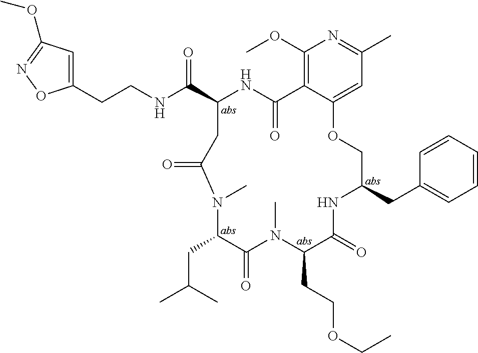 [SMILES: CCOCC[C@@H]1C(=O)N[C@H](Cc2ccccc2)COc2cc(C)nc(OC)c2C(=O)N[C@H](C(=O)NCCc2cc(OC)no2)CC(=O)N(C)[C@@H](CC(C)C)C(=O)N1C] | 877 |
| 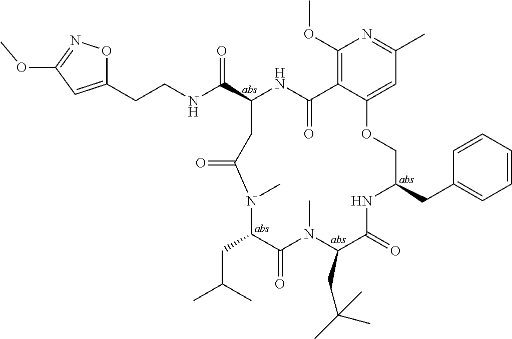 [SMILES: COc1cc(CCNC(=O)[C@@H]2CC(=O)N(C)[C@@H](CC(C)C)C(=O)N(C)[C@H](CC(C)(C)C)C(=O)N[C@H](Cc3ccccc3)COc3cc(C)nc(OC)c3C(=O)N2)on1] | 878 |
| 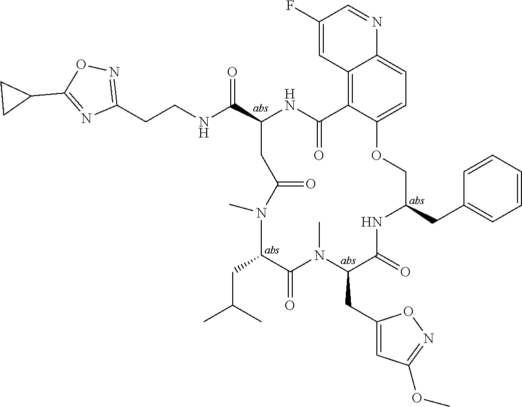 [SMILES: COc1cc(C[C@@H]2C(=O)N[C@H](Cc3ccccc3)COc3ccc4ncc(F)cc4c3C(=O)N[C@H](C(=O)NCCc3noc(C4CC4)n3)CC(=O)N(C)[C@@H](CC(C)C)C(=O)N2C)on1] | 879 |
| 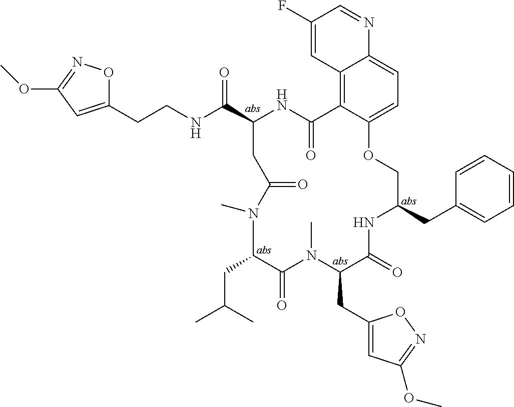 [SMILES: COc1cc(CCNC(=O)[C@@H]2CC(=O)N(C)[C@@H](CC(C)C)C(=O)N(C)[C@H](Cc3cc(OC)no3)C(=O)N[C@H](Cc3ccccc3)COc3ccc4ncc(F)cc4c3C(=O)N2)on1] | 880 |
| 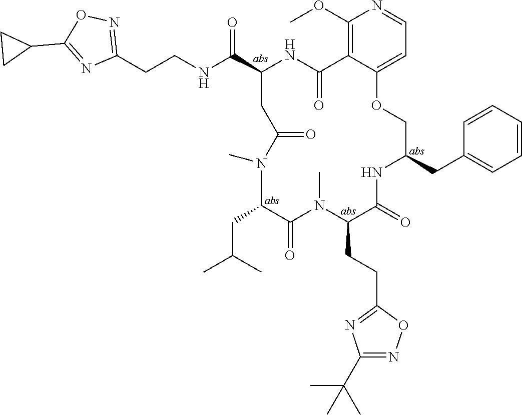 [SMILES: COc1nccc2c1C(=O)N[C@H](C(=O)NCCc1noc(C3CC3)n1)CC(=O)N(C)[C@@H](CC(C)C)C(=O)N(C)[C@H](CCc1nc(C(C)(C)C)no1)C(=O)N[C@H](Cc1ccccc1)CO2] | 881 |
| 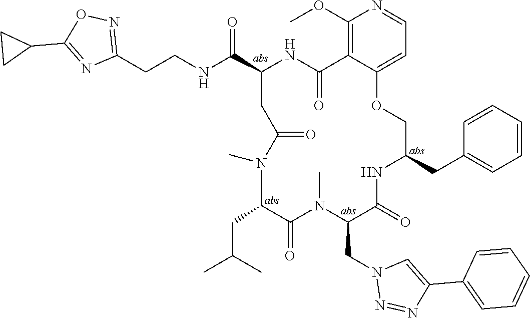 [SMILES: COc1nccc2c1C(=O)N[C@H](C(=O)NCCc1noc(C3CC3)n1)CC(=O)N(C)[C@@H](CC(C)C)C(=O)N(C)[C@H](Cn1cc(-c3ccccc3)nn1)C(=O)N[C@H](Cc1ccccc1)CO2] | 882 |
| 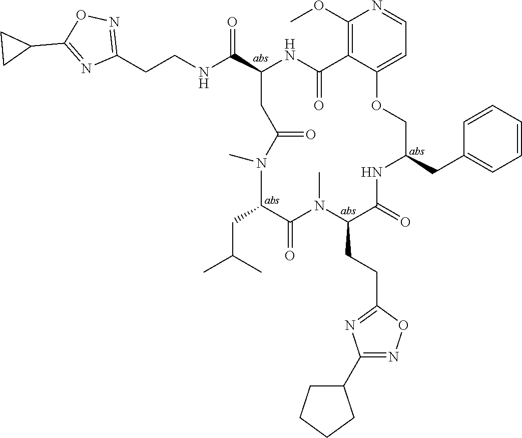 [SMILES: COc1nccc2c1C(=O)N[C@H](C(=O)NCCc1noc(C3CC3)n1)CC(=O)N(C)[C@@H](CC(C)C)C(=O)N(C)[C@H](CCc1nc(C3CCCC3)no1)C(=O)N[C@H](Cc1ccccc1)CO2] | 883 |
| 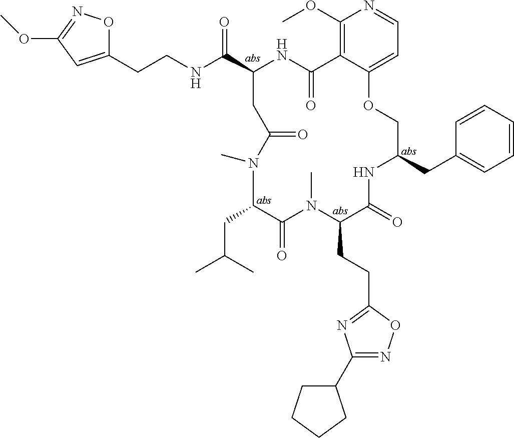 [SMILES: COc1cc(CCNC(=O)[C@@H]2CC(=O)N(C)[C@@H](CC(C)C)C(=O)N(C)[C@H](CCc3nc(C4CCCC4)no3)C(=O)N[C@H](Cc3ccccc3)COc3ccnc(OC)c3C(=O)N2)on1] | 884 |
| 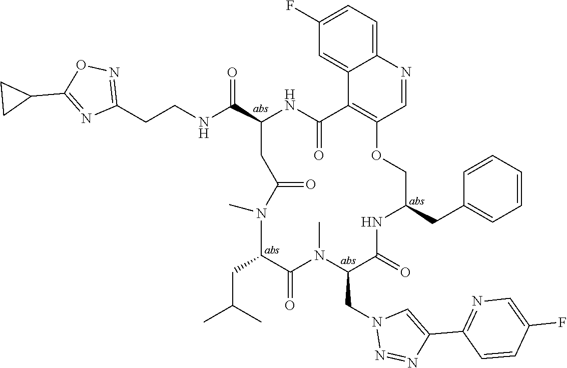 [SMILES: CC(C)C[C@H]1C(=O)N(C)[C@H](Cn2cc(-c3ccc(F)cn3)nn2)C(=O)N[C@H](Cc2ccccc2)COc2cnc3ccc(F)cc3c2C(=O)N[C@H](C(=O)NCCc2noc(C3CC3)n2)CC(=O)N1C] | 885 |
| 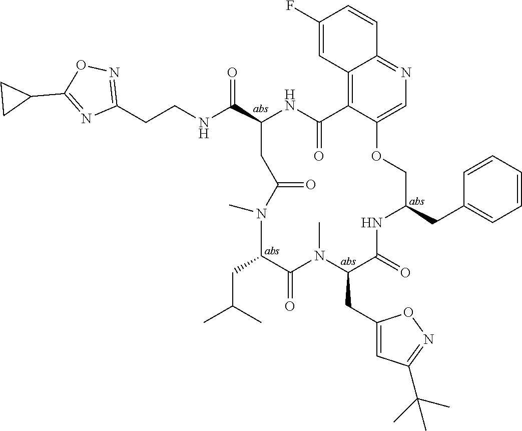 [SMILES: CC(C)C[C@H]1C(=O)N(C)[C@H](Cc2cc(C(C)(C)C)no2)C(=O)N[C@H](Cc2ccccc2)COc2cnc3ccc(F)cc3c2C(=O)N[C@H](C(=O)NCCc2noc(C3CC3)n2)CC(=O)N1C] | 886 |
| 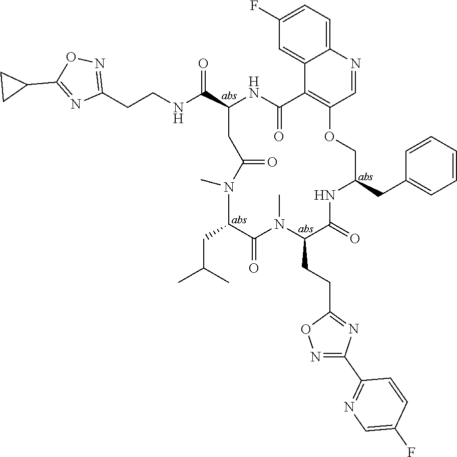 [SMILES: CC(C)C[C@H]1C(=O)N(C)[C@H](CCc2nc(-c3ccc(F)cn3)no2)C(=O)N[C@H](Cc2ccccc2)COc2cnc3ccc(F)cc3c2C(=O)N[C@H](C(=O)NCCc2noc(C3CC3)n2)CC(=O)N1C] | 887 |
| 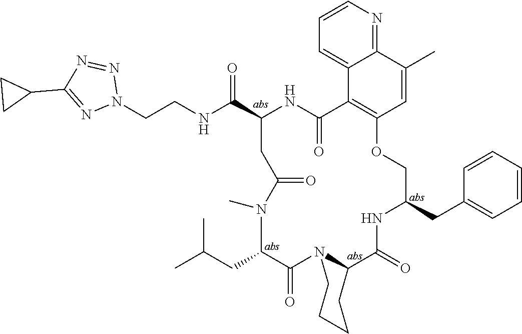 | 888 |
| 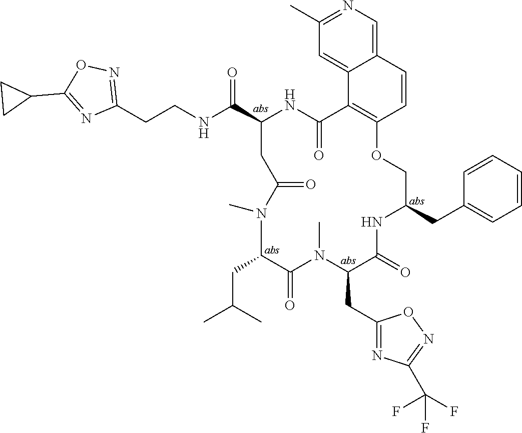 [SMILES: Cc1cc2c3c(ccc2cn1)OC[C@@H](Cc1ccccc1)NC(=O)[C@@H](Cc1nc(C(F)(F)F)no1)N(C)C(=O)[C@H](CC(C)C)N(C)C(=O)C[C@@H](C(=O)NCCc1noc(C2CC2)n1)NC3=O] | 889 |
| 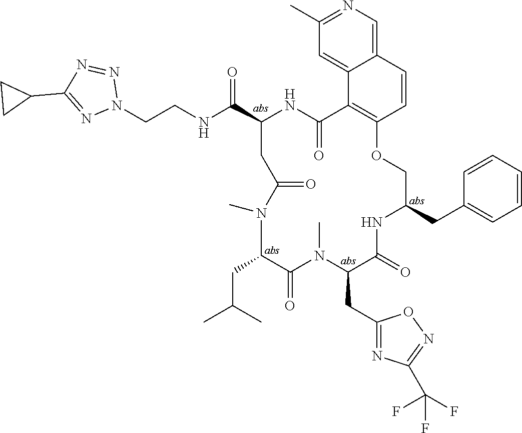 [SMILES: Cc1cc2c3c(ccc2cn1)OC[C@@H](Cc1ccccc1)NC(=O)[C@@H](Cc1nc(C(F)(F)F)no1)N(C)C(=O)[C@H](CC(C)C)N(C)C(=O)C[C@@H](C(=O)NCCn1nnc(C2CC2)n1)NC3=O] | 890 |
| 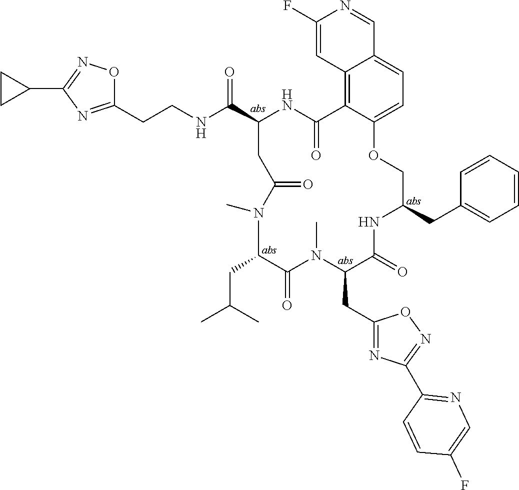 [SMILES: CC(C)C[C@H]1C(=O)N(C)[C@H](Cc2nc(-c3ccc(F)cn3)no2)C(=O)N[C@H](Cc2ccccc2)COc2ccc3cnc(F)cc3c2C(=O)N[C@H](C(=O)NCCc2nc(C3CC3)no2)CC(=O)N1C] | 891 |
| 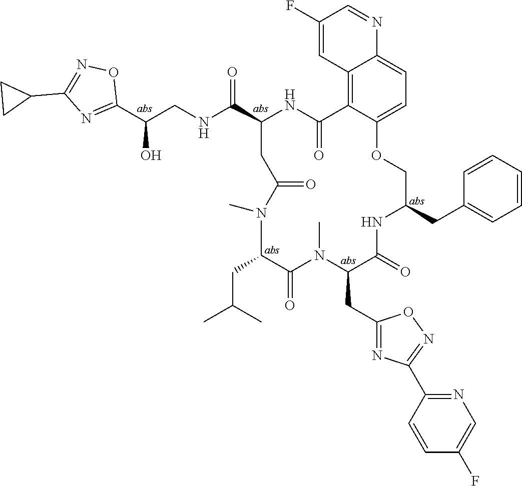 [SMILES: CC(C)C[C@H]1C(=O)N(C)[C@H](Cc2nc(-c3ccc(F)cn3)no2)C(=O)N[C@H](Cc2ccccc2)COc2ccc3ncc(F)cc3c2C(=O)N[C@H](C(=O)NC[C@@H](O)c2nc(C3CC3)no2)CC(=O)N1C] | 892 |
| 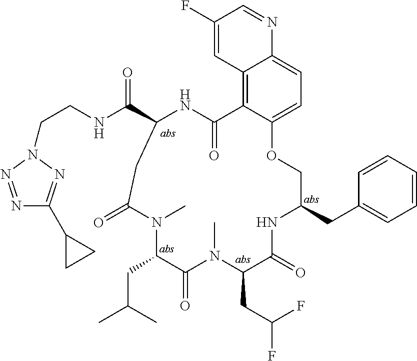 [SMILES: CC(C)C[C@H]1C(=O)N(C)[C@H](CC(F)F)C(=O)N[C@H](Cc2ccccc2)COc2ccc3ncc(F)cc3c2C(=O)N[C@H](C(=O)NCCn2nnc(C3CC3)n2)CC(=O)N1C] | 893 |
| 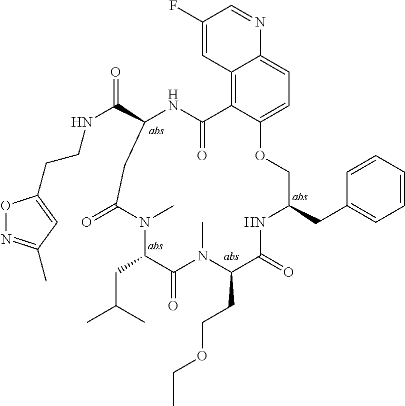 [SMILES: CCOCC[C@@H]1C(=O)N[C@H](Cc2ccccc2)COc2ccc3ncc(F)cc3c2C(=O)N[C@H](C(=O)NCCc2cc(C)no2)CC(=O)N(C)[C@@H](CC(C)C)C(=O)N1C] | 894 |
| 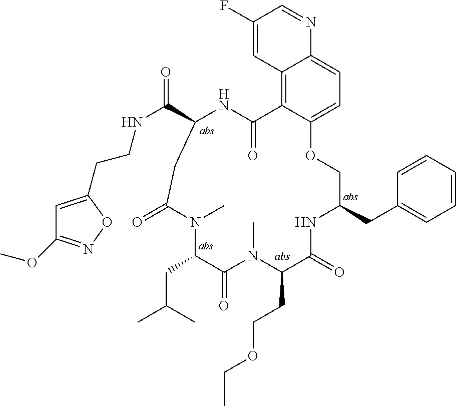 [SMILES: CCOCC[C@@H]1C(=O)N[C@H](Cc2ccccc2)COc2ccc3ncc(F)cc3c2C(=O)N[C@H](C(=O)NCCc2cc(OC)no2)CC(=O)N(C)[C@@H](CC(C)C)C(=O)N1C] | 895 |
| 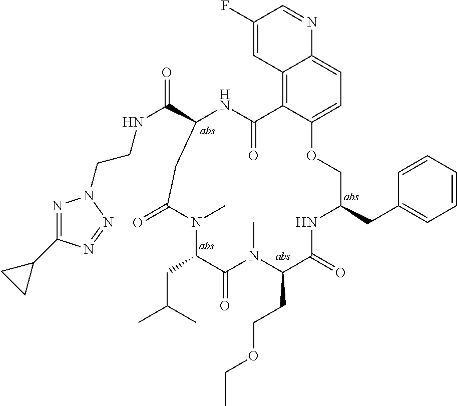 [SMILES: CCOCC[C@@H]1C(=O)N[C@H](Cc2ccccc2)COc2ccc3ncc(F)cc3c2C(=O)N[C@H](C(=O)NCCn2nnc(C3CC3)n2)CC(=O)N(C)[C@@H](CC(C)C)C(=O)N1C] | 896 |
| 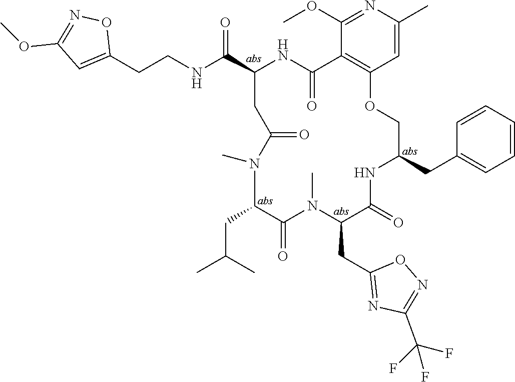 [SMILES: COc1cc(CCNC(=O)[C@@H]2CC(=O)N(C)[C@@H](CC(C)C)C(=O)N(C)[C@H](Cc3nc(C(F)(F)F)no3)C(=O)N[C@H](Cc3ccccc3)COc3cc(C)nc(OC)c3C(=O)N2)on1] | 897 |
| 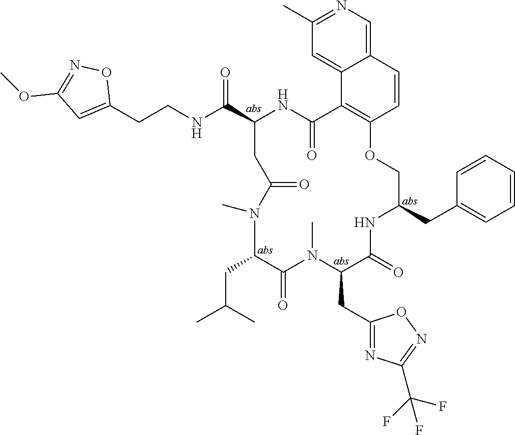 [SMILES: COc1cc(CCNC(=O)[C@@H]2CC(=O)N(C)[C@@H](CC(C)C)C(=O)N(C)[C@H](Cc3nc(C(F)(F)F)no3)C(=O)N[C@H](Cc3ccccc3)COc3ccc4cnc(C)cc4c3C(=O)N2)on1] | 898 |
| 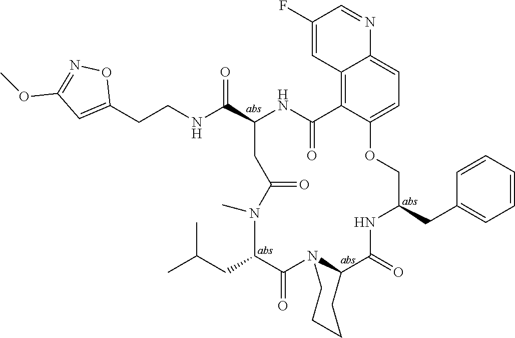 | 899 |
| 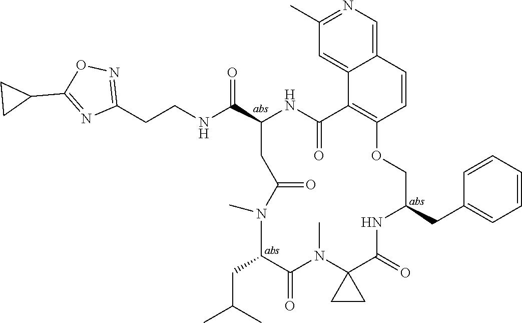 [SMILES: Cc1cc2c3c(ccc2cn1)OC[C@@H](Cc1ccccc1)NC(=O)C1(CC1)N(C)C(=O)[C@H](CC(C)C)N(C)C(=O)C[C@@H](C(=O)NCCc1noc(C2CC2)n1)NC3=O] | 900 |
| 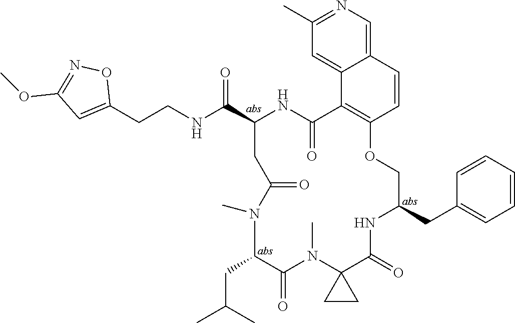 [SMILES: COc1cc(CCNC(=O)[C@@H]2CC(=O)N(C)[C@@H](CC(C)C)C(=O)N(C)C3(CC3)C(=O)N[C@H](Cc3ccccc3)COc3ccc4cnc(C)cc4c3C(=O)N2)on1] | 901 |
| 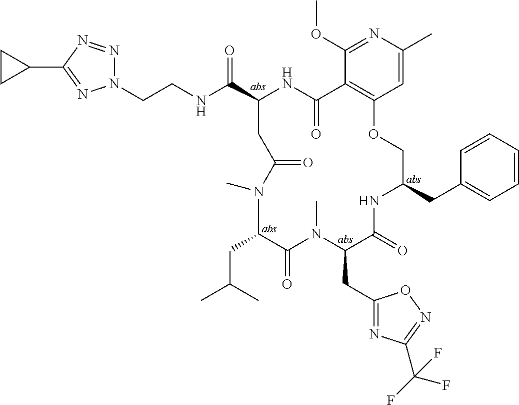 [SMILES: COc1nc(C)cc2c1C(=O)N[C@H](C(=O)NCCn1nnc(C3CC3)n1)CC(=O)N(C)[C@@H](CC(C)C)C(=O)N(C)[C@H](Cc1nc(C(F)(F)F)no1)C(=O)N[C@H](Cc1ccccc1)CO2] | 902 |
| 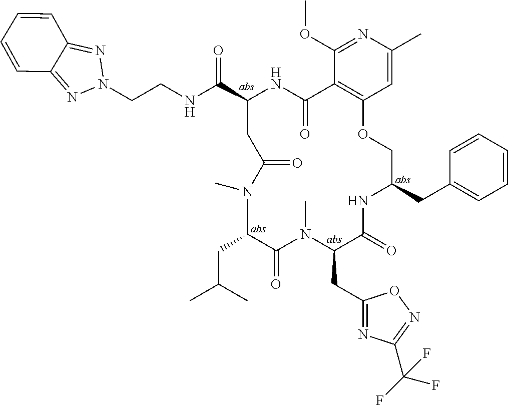 [SMILES: COc1nc(C)cc2c1C(=O)N[C@H](C(=O)NCCn1nc3ccccc3n1)CC(=O)N(C)[C@@H](CC(C)C)C(=O)N(C)[C@H](Cc1nc(C(F)(F)F)no1)C(=O)N[C@H](Cc1ccccc1)CO2] | 903 |
| 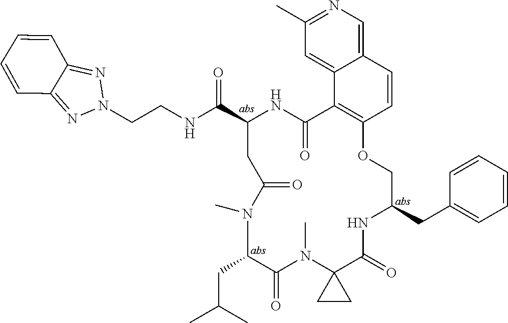 [SMILES: Cc1cc2c3c(ccc2cn1)OC[C@@H](Cc1ccccc1)NC(=O)C1(CC1)N(C)C(=O)[C@H](CC(C)C)N(C)C(=O)C[C@@H](C(=O)NCCn1nc2ccccc2n1)NC3=O] | 904 |
| 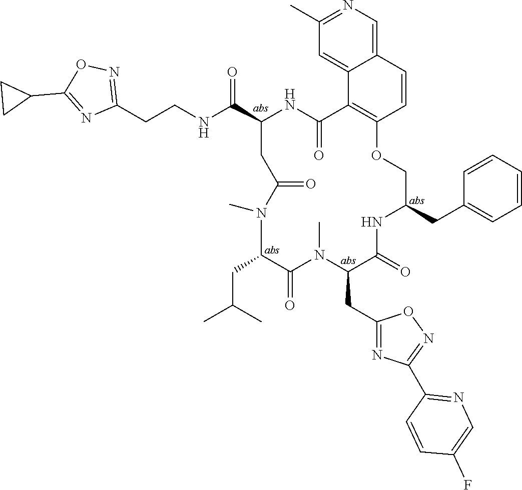 [SMILES: Cc1cc2c3c(ccc2cn1)OC[C@@H](Cc1ccccc1)NC(=O)[C@@H](Cc1nc(-c2ccc(F)cn2)no1)N(C)C(=O)[C@H](CC(C)C)N(C)C(=O)C[C@@H](C(=O)NCCc1noc(C2CC2)n1)NC3=O] | 905 |
| 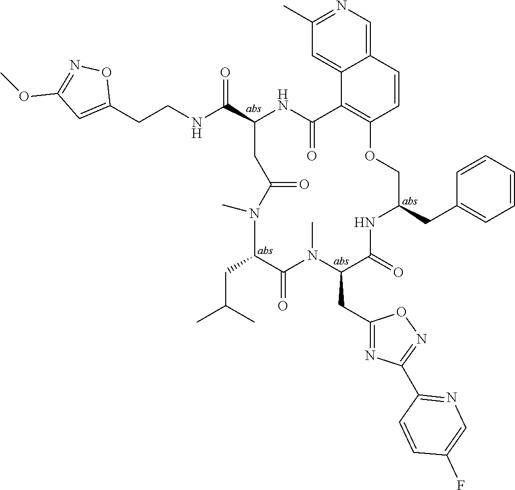 [SMILES: COc1cc(CCNC(=O)[C@@H]2CC(=O)N(C)[C@@H](CC(C)C)C(=O)N(C)[C@H](Cc3nc(-c4ccc(F)cn4)no3)C(=O)N[C@H](Cc3ccccc3)COc3ccc4cnc(C)cc4c3C(=O)N2)on1] | 906 |
| 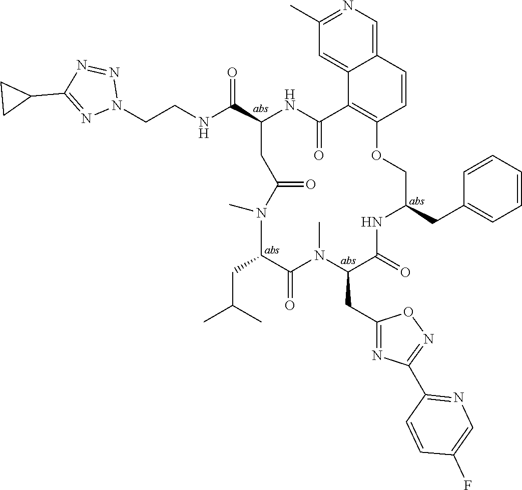 [SMILES: Cc1cc2c3c(ccc2cn1)OC[C@@H](Cc1ccccc1)NC(=O)[C@@H](Cc1nc(-c2ccc(F)cn2)no1)N(C)C(=O)[C@H](CC(C)C)N(C)C(=O)C[C@@H](C(=O)NCCn1nnc(C2CC2)n1)NC3=O] | 907 |
| 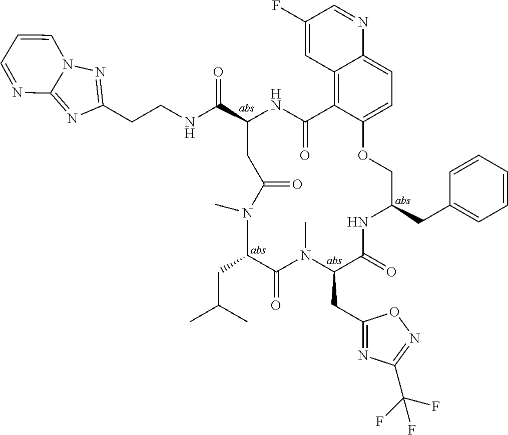 [SMILES: CC(C)C[C@H]1C(=O)N(C)[C@H](Cc2nc(C(F)(F)F)no2)C(=O)N[C@H](Cc2ccccc2)COc2ccc3ncc(F)cc3c2C(=O)N[C@H](C(=O)NCCc2nc3ncccn3n2)CC(=O)N1C] | 908 |
| 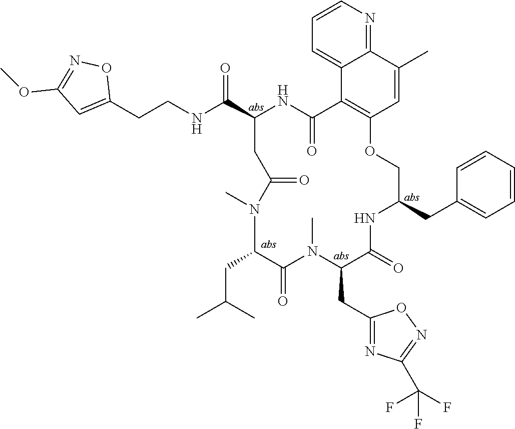 [SMILES: COc1cc(CCNC(=O)[C@@H]2CC(=O)N(C)[C@@H](CC(C)C)C(=O)N(C)[C@H](Cc3nc(C(F)(F)F)no3)C(=O)N[C@H](Cc3ccccc3)COc3cc(C)c4ncccc4c3C(=O)N2)on1] | 909 |
| 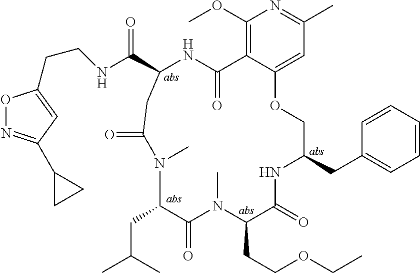 [SMILES: CCOCC[C@@H]1C(=O)N[C@H](Cc2ccccc2)COc2cc(C)nc(OC)c2C(=O)N[C@H](C(=O)NCCc2cc(C3CC3)no2)CC(=O)N(C)[C@@H](CC(C)C)C(=O)N1C] | 910 |
| 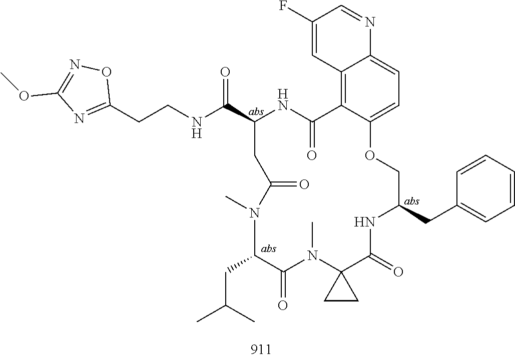 [SMILES: COc1noc(CCNC(=O)[C@@H]2CC(=O)N(C)[C@@H](CC(C)C)C(=O)N(C)C3(CC3)C(=O)N[C@H](Cc3ccccc3)COc3ccc4ncc(F)cc4c3C(=O)N2)n1] | 911 |
| 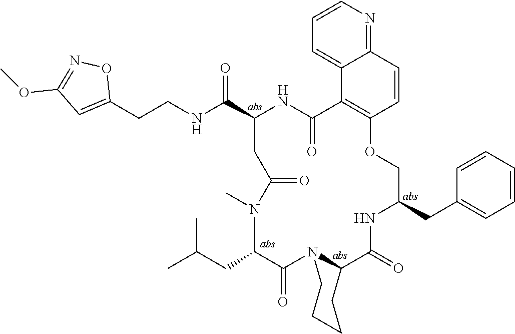 | 912 |
| 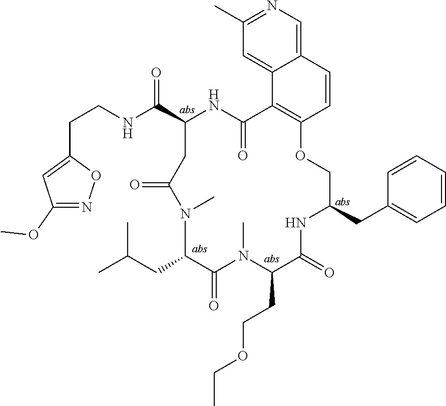 [SMILES: CCOCC[C@@H]1C(=O)N[C@H](Cc2ccccc2)COc2ccc3cnc(C)cc3c2C(=O)N[C@H](C(=O)NCCc2cc(OC)no2)CC(=O)N(C)[C@@H](CC(C)C)C(=O)N1C] | 913 |
| 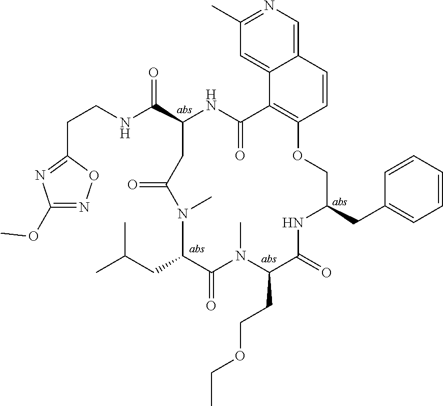 [SMILES: CCOCC[C@@H]1C(=O)N[C@H](Cc2ccccc2)COc2ccc3cnc(C)cc3c2C(=O)N[C@H](C(=O)NCCc2nc(OC)no2)CC(=O)N(C)[C@@H](CC(C)C)C(=O)N1C] | 914 |
| 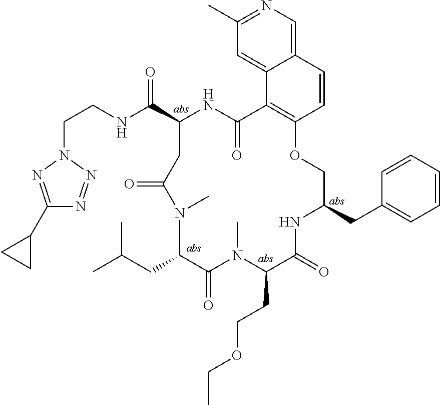 [SMILES: CCOCC[C@@H]1C(=O)N[C@H](Cc2ccccc2)COc2ccc3cnc(C)cc3c2C(=O)N[C@H](C(=O)NCCn2nnc(C3CC3)n2)CC(=O)N(C)[C@@H](CC(C)C)C(=O)N1C] | 915 |
| 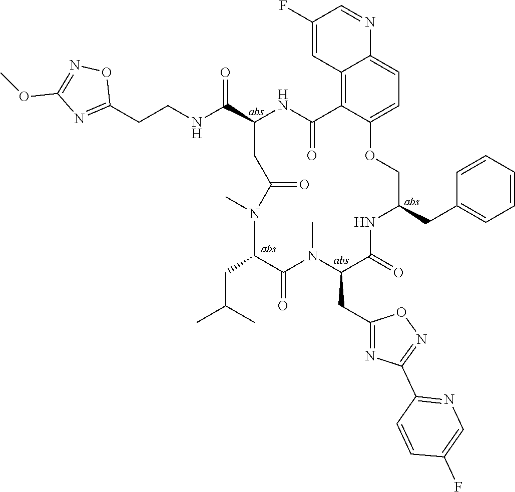 [SMILES: COc1noc(CCNC(=O)[C@@H]2CC(=O)N(C)[C@@H](CC(C)C)C(=O)N(C)[C@H](Cc3nc(-c4ccc(F)cn4)no3)C(=O)N[C@H](Cc3ccccc3)COc3ccc4ncc(F)cc4c3C(=O)N2)n1] | 916 |
| 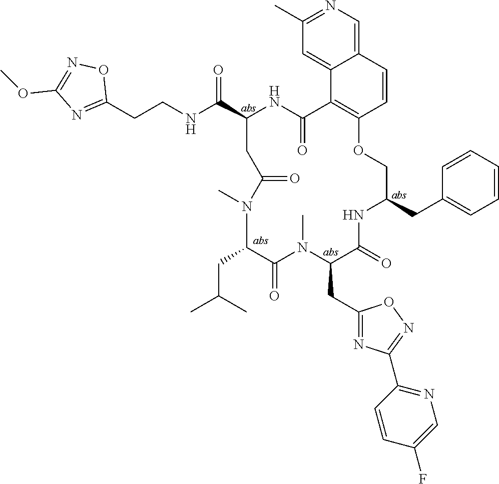 [SMILES: COc1noc(CCNC(=O)[C@@H]2CC(=O)N(C)[C@@H](CC(C)C)C(=O)N(C)[C@H](Cc3nc(-c4ccc(F)cn4)no3)C(=O)N[C@H](Cc3ccccc3)COc3ccc4cnc(C)cc4c3C(=O)N2)n1] | 917 |
| 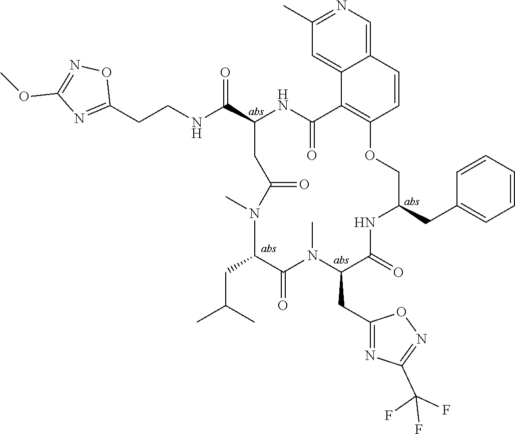 [SMILES: COc1noc(CCNC(=O)[C@@H]2CC(=O)N(C)[C@@H](CC(C)C)C(=O)N(C)[C@H](Cc3nc(C(F)(F)F)no3)C(=O)N[C@H](Cc3ccccc3)COc3ccc4cnc(C)cc4c3C(=O)N2)n1] | 918 |
| 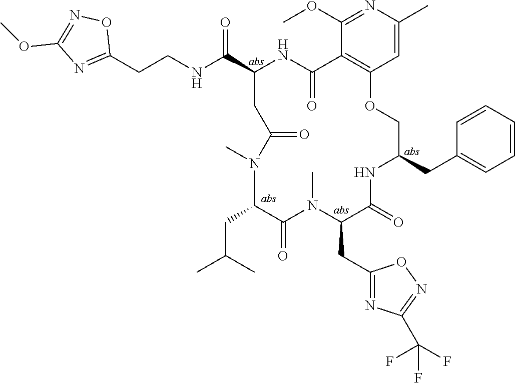 [SMILES: COc1noc(CCNC(=O)[C@@H]2CC(=O)N(C)[C@@H](CC(C)C)C(=O)N(C)[C@H](Cc3nc(C(F)(F)F)no3)C(=O)N[C@H](Cc3ccccc3)COc3cc(C)nc(OC)c3C(=O)N2)n1] | 919 |
| 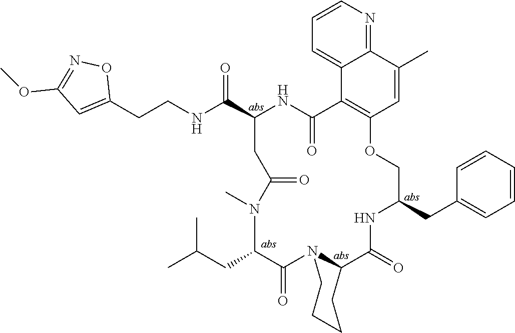 | 920 |
| 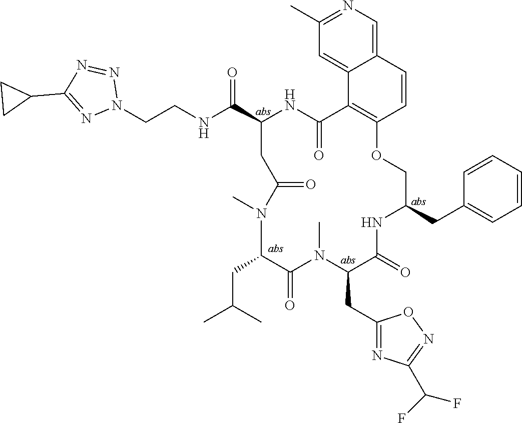 [SMILES: Cc1cc2c3c(ccc2cn1)OC[C@@H](Cc1ccccc1)NC(=O)[C@@H](Cc1nc(C(F)F)no1)N(C)C(=O)[C@H](CC(C)C)N(C)C(=O)C[C@@H](C(=O)NCCn1nnc(C2CC2)n1)NC3=O] | 921 |
| 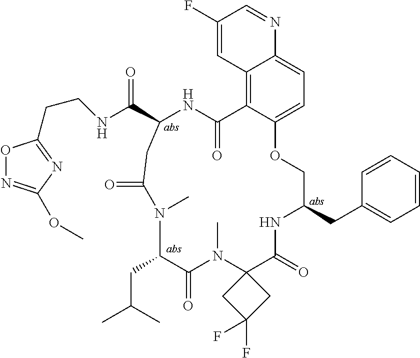 [SMILES: COc1noc(CCNC(=O)[C@@H]2CC(=O)N(C)[C@@H](CC(C)C)C(=O)N(C)C3(CC(F)(F)C3)C(=O)N[C@H](Cc3ccccc3)COc3ccc4ncc(F)cc4c3C(=O)N2)n1] | 922 |
| 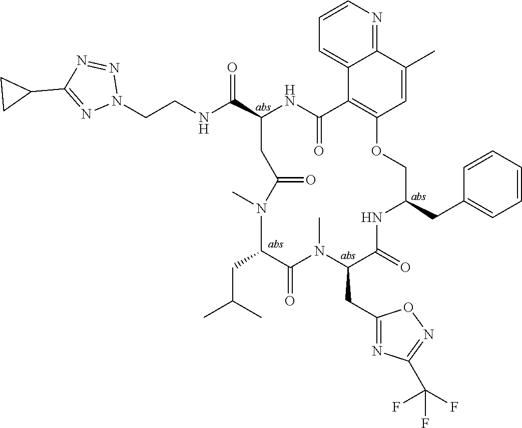 [SMILES: Cc1cc2c(c3cccnc13)C(=O)N[C@H](C(=O)NCCn1nnc(C3CC3)n1)CC(=O)N(C)[C@@H](CC(C)C)C(=O)N(C)[C@H](Cc1nc(C(F)(F)F)no1)C(=O)N[C@H](Cc1ccccc1)CO2] | 923 |
| 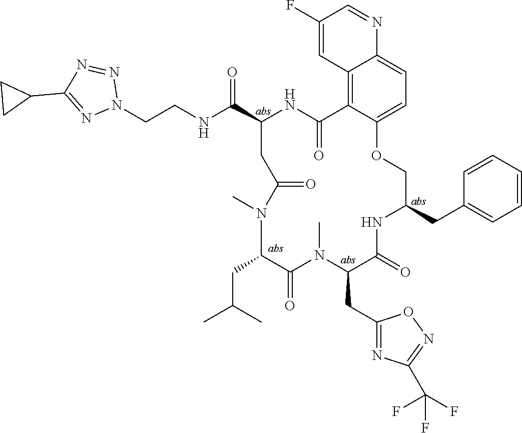 [SMILES: CC(C)C[C@H]1C(=O)N(C)[C@H](Cc2nc(C(F)(F)F)no2)C(=O)N[C@H](Cc2ccccc2)COc2ccc3ncc(F)cc3c2C(=O)N[C@H](C(=O)NCCn2nnc(C3CC3)n2)CC(=O)N1C] | 924 |
| 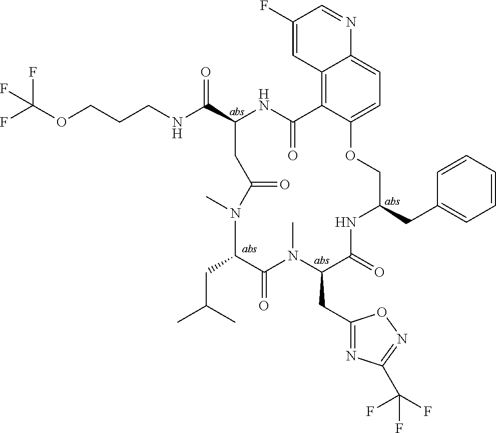 [SMILES: CC(C)C[C@H]1C(=O)N(C)[C@H](Cc2nc(C(F)(F)F)no2)C(=O)N[C@H](Cc2ccccc2)COc2ccc3ncc(F)cc3c2C(=O)N[C@H](C(=O)NCCCOC(F)(F)F)CC(=O)N1C] | 925 |
| 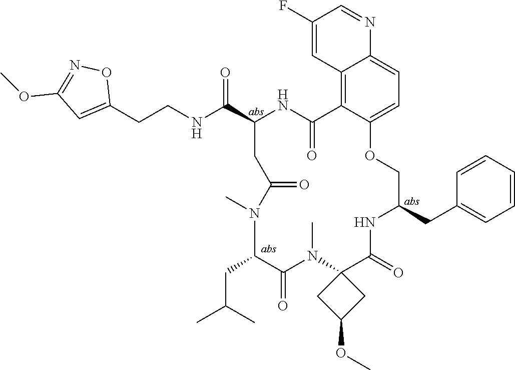 [SMILES: COc1cc(CCNC(=O)[C@@H]2CC(=O)N(C)[C@@H](CC(C)C)C(=O)N(C)[C@]3(C[C@H](OC)C3)C(=O)N[C@H](Cc3ccccc3)COc3ccc4ncc(F)cc4c3C(=O)N2)on1] | 926 |
| 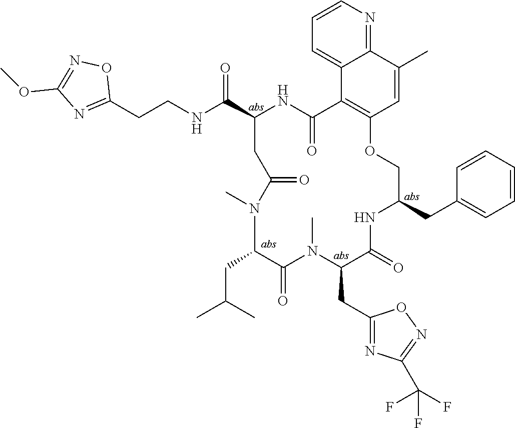 [SMILES: COc1noc(CCNC(=O)[C@@H]2CC(=O)N(C)[C@@H](CC(C)C)C(=O)N(C)[C@H](Cc3nc(C(F)(F)F)no3)C(=O)N[C@H](Cc3ccccc3)COc3cc(C)c4ncccc4c3C(=O)N2)n1] | 927 |
| 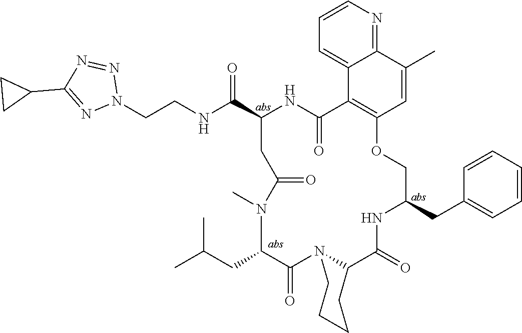 | 928 |
| 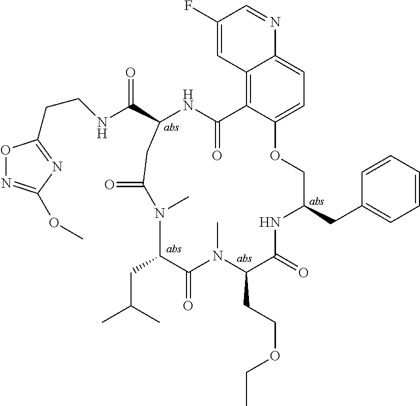 [SMILES: CCOCC[C@@H]1C(=O)N[C@H](Cc2ccccc2)COc2ccc3ncc(F)cc3c2C(=O)N[C@H](C(=O)NCCc2nc(OC)no2)CC(=O)N(C)[C@@H](CC(C)C)C(=O)N1C] | 929 |
| 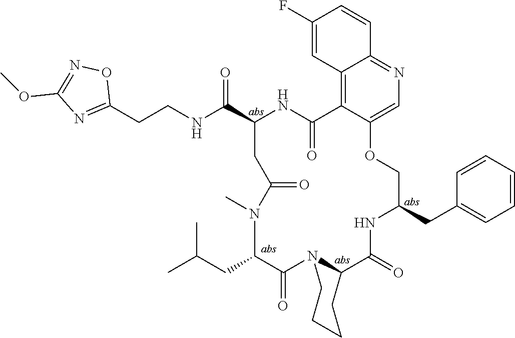 | 930 |
| 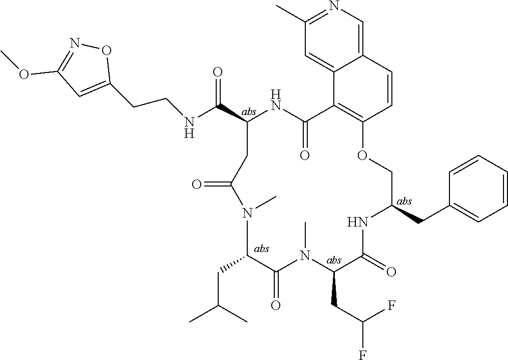 [SMILES: COc1cc(CCNC(=O)[C@@H]2CC(=O)N(C)[C@@H](CC(C)C)C(=O)N(C)[C@H](CC(F)F)C(=O)N[C@H](Cc3ccccc3)COc3ccc4cnc(C)cc4c3C(=O)N2)on1] | 931 |
| 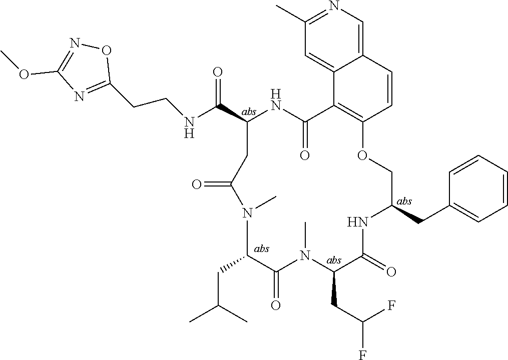 [SMILES: COc1noc(CCNC(=O)[C@@H]2CC(=O)N(C)[C@@H](CC(C)C)C(=O)N(C)[C@H](CC(F)F)C(=O)N[C@H](Cc3ccccc3)COc3ccc4cnc(C)cc4c3C(=O)N2)n1] | 932 |
| 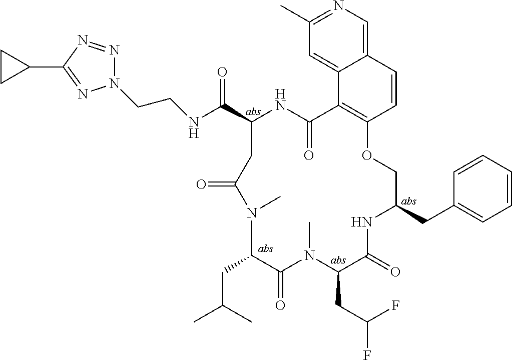 [SMILES: Cc1cc2c3c(ccc2cn1)OC[C@@H](Cc1ccccc1)NC(=O)[C@@H](CC(F)F)N(C)C(=O)[C@H](CC(C)C)N(C)C(=O)C[C@@H](C(=O)NCCn1nnc(C2CC2)n1)NC3=O] | 933 |
| 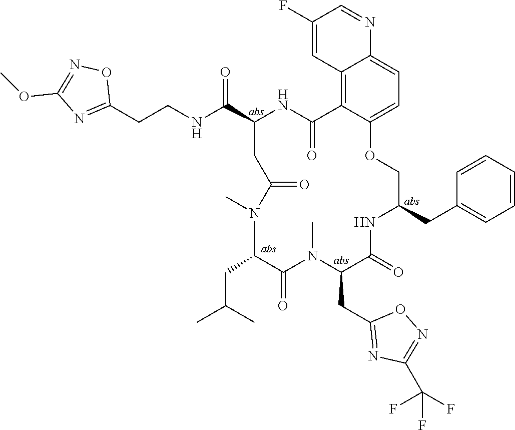 [SMILES: COc1noc(CCNC(=O)[C@@H]2CC(=O)N(C)[C@@H](CC(C)C)C(=O)N(C)[C@H](Cc3nc(C(F)(F)F)no3)C(=O)N[C@H](Cc3ccccc3)COc3ccc4ncc(F)cc4c3C(=O)N2)n1] | 934 |
| 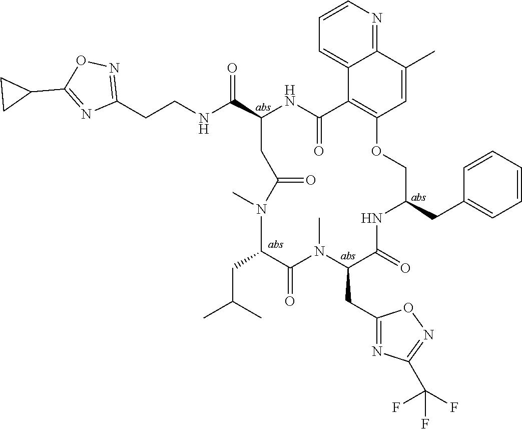 [SMILES: Cc1cc2c(c3cccnc13)C(=O)N[C@H](C(=O)NCCc1noc(C3CC3)n1)CC(=O)N(C)[C@@H](CC(C)C)C(=O)N(C)[C@H](Cc1nc(C(F)(F)F)no1)C(=O)N[C@H](Cc1ccccc1)CO2] | 935 |
| 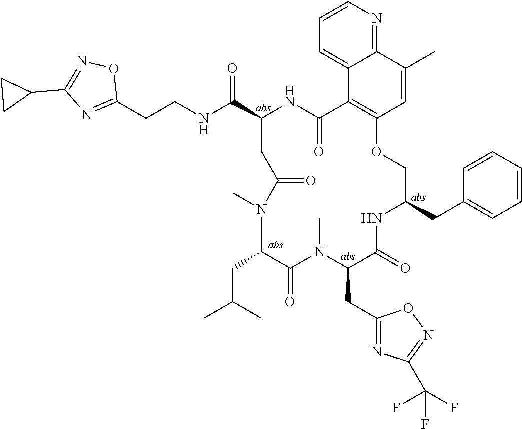 [SMILES: Cc1cc2c(c3cccnc13)C(=O)N[C@H](C(=O)NCCc1nc(C3CC3)no1)CC(=O)N(C)[C@@H](CC(C)C)C(=O)N(C)[C@H](Cc1nc(C(F)(F)F)no1)C(=O)N[C@H](Cc1ccccc1)CO2] | 936 |
| 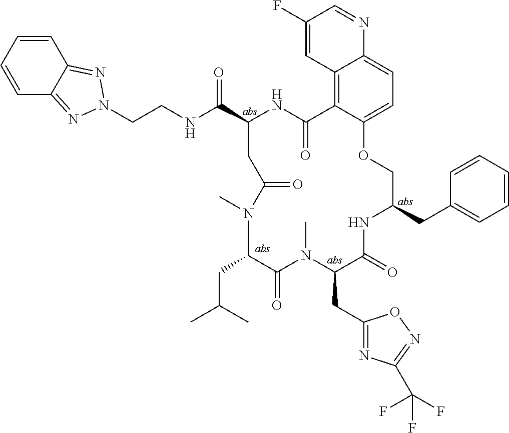 [SMILES: CC(C)C[C@H]1C(=O)N(C)[C@H](Cc2nc(C(F)(F)F)no2)C(=O)N[C@H](Cc2ccccc2)COc2ccc3ncc(F)cc3c2C(=O)N[C@H](C(=O)NCCn2nc3ccccc3n2)CC(=O)N1C] | 937 |
| 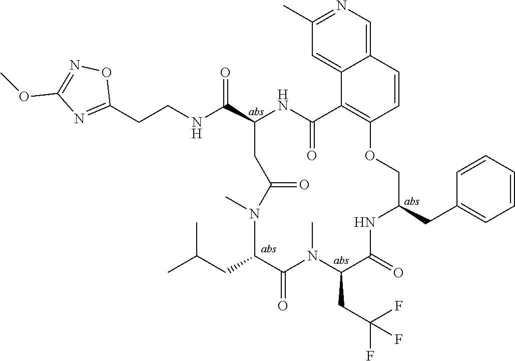 [SMILES: COc1noc(CCNC(=O)[C@@H]2CC(=O)N(C)[C@@H](CC(C)C)C(=O)N(C)[C@H](CC(F)(F)F)C(=O)N[C@H](Cc3ccccc3)COc3ccc4cnc(C)cc4c3C(=O)N2)n1] | 938 |
| 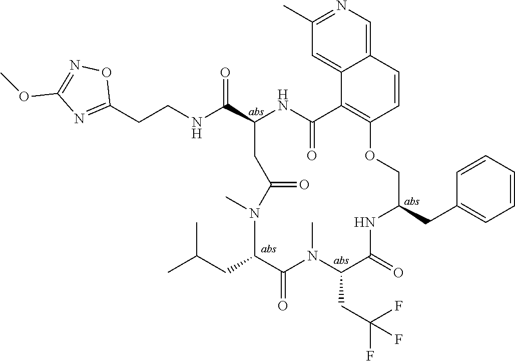 [SMILES: COc1noc(CCNC(=O)[C@@H]2CC(=O)N(C)[C@@H](CC(C)C)C(=O)N(C)[C@@H](CC(F)(F)F)C(=O)N[C@H](Cc3ccccc3)COc3ccc4cnc(C)cc4c3C(=O)N2)n1] | 939 |
| 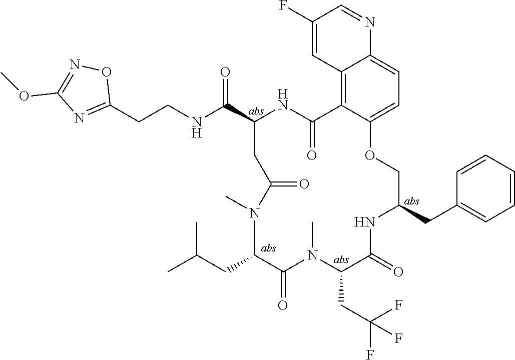 [SMILES: COc1noc(CCNC(=O)[C@@H]2CC(=O)N(C)[C@@H](CC(C)C)C(=O)N(C)[C@@H](CC(F)(F)F)C(=O)N[C@H](Cc3ccccc3)COc3ccc4ncc(F)cc4c3C(=O)N2)n1] | 940 |
| 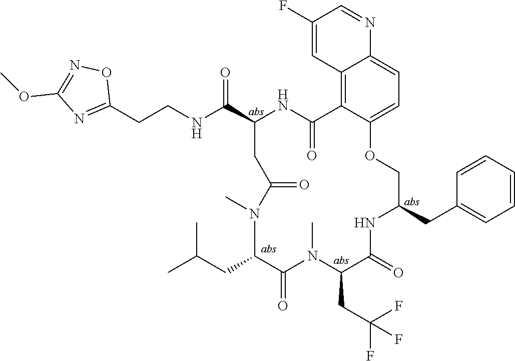 [SMILES: COc1noc(CCNC(=O)[C@@H]2CC(=O)N(C)[C@@H](CC(C)C)C(=O)N(C)[C@H](CC(F)(F)F)C(=O)N[C@H](Cc3ccccc3)COc3ccc4ncc(F)cc4c3C(=O)N2)n1] | 941 |
| 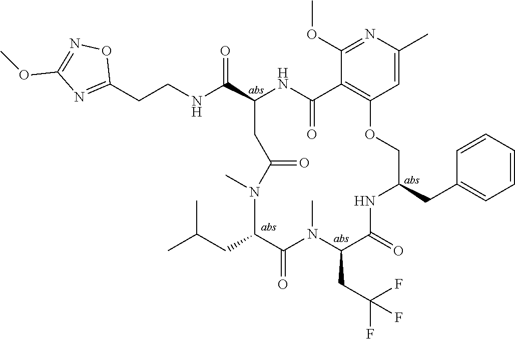 [SMILES: COc1noc(CCNC(=O)[C@@H]2CC(=O)N(C)[C@@H](CC(C)C)C(=O)N(C)[C@H](CC(F)(F)F)C(=O)N[C@H](Cc3ccccc3)COc3cc(C)nc(OC)c3C(=O)N2)n1] | 942 |
| 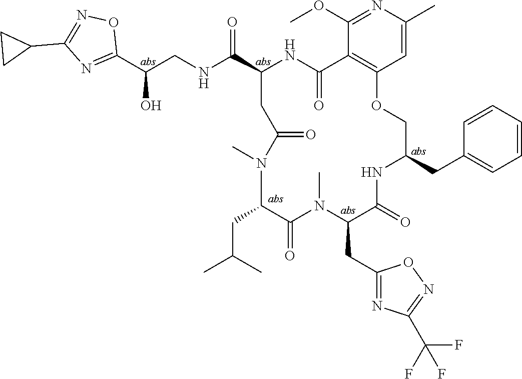 [SMILES: COc1nc(C)cc2c1C(=O)N[C@H](C(=O)NC[C@@H](O)c1nc(C3CC3)no1)CC(=O)N(C)[C@@H](CC(C)C)C(=O)N(C)[C@H](Cc1nc(C(F)(F)F)no1)C(=O)N[C@H](Cc1ccccc1)CO2] | 943 |
| 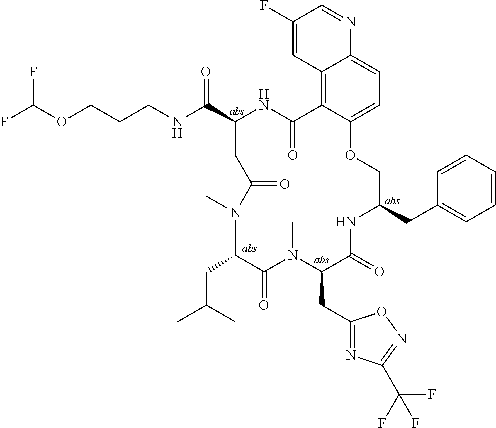 [SMILES: CC(C)C[C@H]1C(=O)N(C)[C@H](Cc2nc(C(F)(F)F)no2)C(=O)N[C@H](Cc2ccccc2)COc2ccc3ncc(F)cc3c2C(=O)N[C@H](C(=O)NCCCOC(F)F)CC(=O)N1C] | 944 |
| 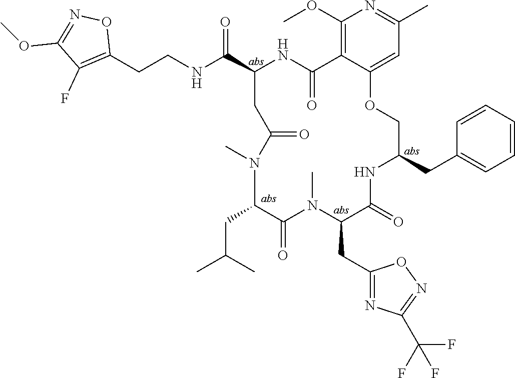 [SMILES: COc1noc(CCNC(=O)[C@@H]2CC(=O)N(C)[C@@H](CC(C)C)C(=O)N(C)[C@H](Cc3nc(C(F)(F)F)no3)C(=O)N[C@H](Cc3ccccc3)COc3cc(C)nc(OC)c3C(=O)N2)c1F] | 945 |
| 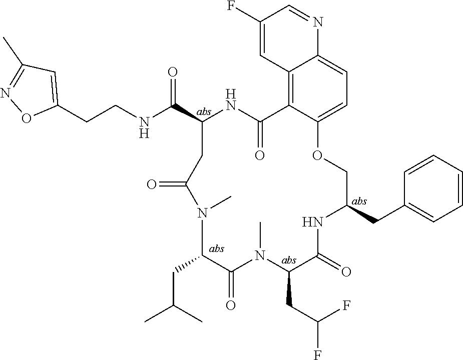 [SMILES: Cc1cc(CCNC(=O)[C@@H]2CC(=O)N(C)[C@@H](CC(C)C)C(=O)N(C)[C@H](CC(F)F)C(=O)N[C@H](Cc3ccccc3)COc3ccc4ncc(F)cc4c3C(=O)N2)on1] | 946 |
| 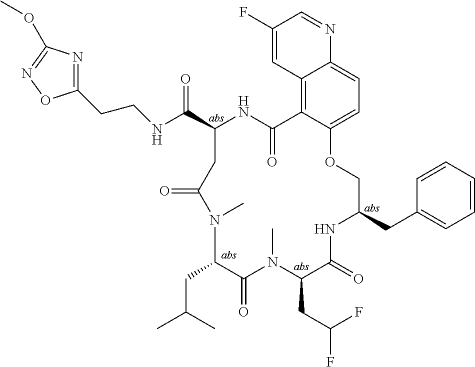 [SMILES: COc1noc(CCNC(=O)[C@@H]2CC(=O)N(C)[C@@H](CC(C)C)C(=O)N(C)[C@H](CC(F)F)C(=O)N[C@H](Cc3ccccc3)COc3ccc4ncc(F)cc4c3C(=O)N2)n1] | 947 |
| 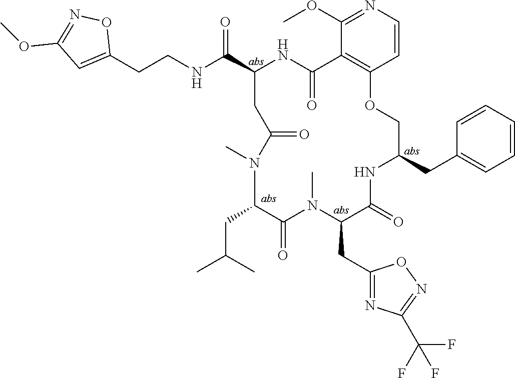 [SMILES: COc1cc(CCNC(=O)[C@@H]2CC(=O)N(C)[C@@H](CC(C)C)C(=O)N(C)[C@H](Cc3nc(C(F)(F)F)no3)C(=O)N[C@H](Cc3ccccc3)COc3ccnc(OC)c3C(=O)N2)on1] | 948 |
| 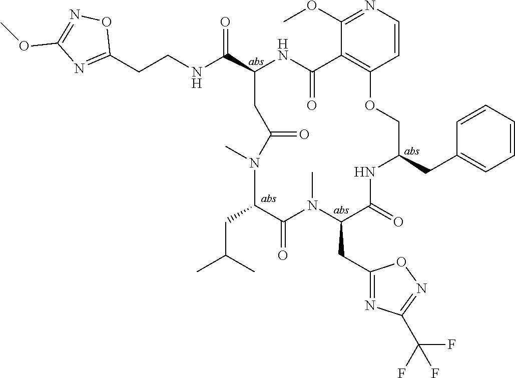 [SMILES: COc1noc(CCNC(=O)[C@@H]2CC(=O)N(C)[C@@H](CC(C)C)C(=O)N(C)[C@H](Cc3nc(C(F)(F)F)no3)C(=O)N[C@H](Cc3ccccc3)COc3ccnc(OC)c3C(=O)N2)n1] | 949 |
| 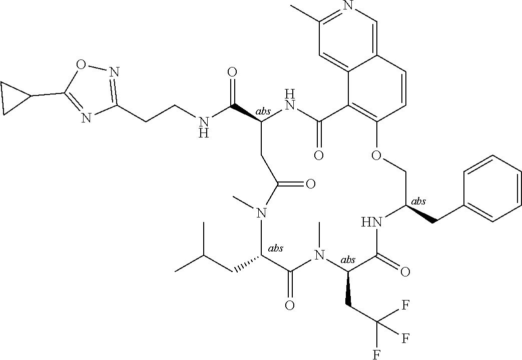 [SMILES: Cc1cc2c3c(ccc2cn1)OC[C@@H](Cc1ccccc1)NC(=O)[C@@H](CC(F)(F)F)N(C)C(=O)[C@H](CC(C)C)N(C)C(=O)C[C@@H](C(=O)NCCc1noc(C2CC2)n1)NC3=O] | 950 |
| 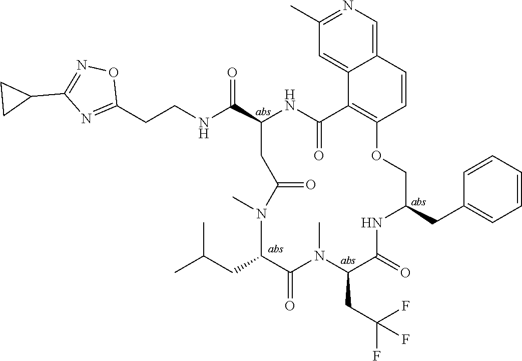 [SMILES: Cc1cc2c3c(ccc2cn1)OC[C@@H](Cc1ccccc1)NC(=O)[C@@H](CC(F)(F)F)N(C)C(=O)[C@H](CC(C)C)N(C)C(=O)C[C@@H](C(=O)NCCc1nc(C2CC2)no1)NC3=O] | 951 |
| 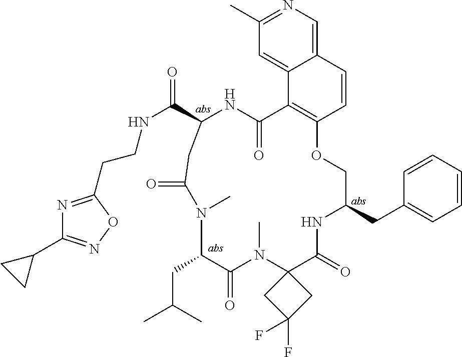 [SMILES: Cc1cc2c3c(ccc2cn1)OC[C@@H](Cc1ccccc1)NC(=O)C1(CC(F)(F)C1)N(C)C(=O)[C@H](CC(C)C)N(C)C(=O)C[C@@H](C(=O)NCCc1nc(C2CC2)no1)NC3=O] | 952 |
| 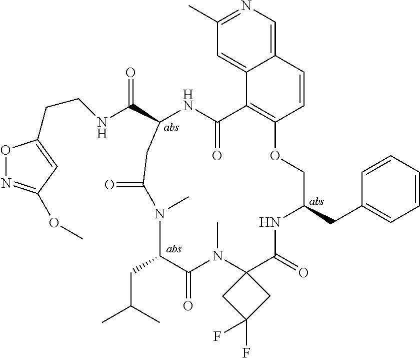 [SMILES: COc1cc(CCNC(=O)[C@@H]2CC(=O)N(C)[C@@H](CC(C)C)C(=O)N(C)C3(CC(F)(F)C3)C(=O)N[C@H](Cc3ccccc3)COc3ccc4cnc(C)cc4c3C(=O)N2)on1] | 953 |
| 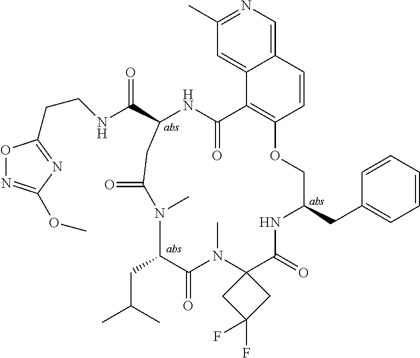 [SMILES: COc1noc(CCNC(=O)[C@@H]2CC(=O)N(C)[C@@H](CC(C)C)C(=O)N(C)C3(CC(F)(F)C3)C(=O)N[C@H](Cc3ccccc3)COc3ccc4cnc(C)cc4c3C(=O)N2)n1] | 954 |
| 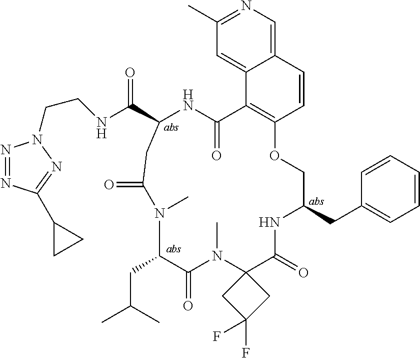 [SMILES: Cc1cc2c3c(ccc2cn1)OC[C@@H](Cc1ccccc1)NC(=O)C1(CC(F)(F)C1)N(C)C(=O)[C@H](CC(C)C)N(C)C(=O)C[C@@H](C(=O)NCCn1nnc(C2CC2)n1)NC3=O] | 955 |
| 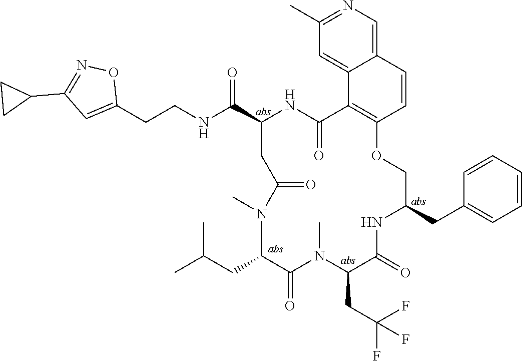 [SMILES: Cc1cc2c3c(ccc2cn1)OC[C@@H](Cc1ccccc1)NC(=O)[C@@H](CC(F)(F)F)N(C)C(=O)[C@H](CC(C)C)N(C)C(=O)C[C@@H](C(=O)NCCc1cc(C2CC2)no1)NC3=O] | 956 |
| 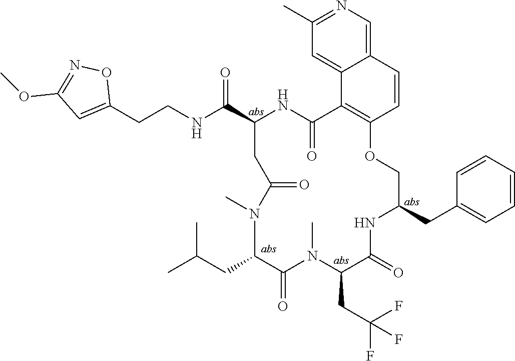 [SMILES: COc1cc(CCNC(=O)[C@@H]2CC(=O)N(C)[C@@H](CC(C)C)C(=O)N(C)[C@H](CC(F)(F)F)C(=O)N[C@H](Cc3ccccc3)COc3ccc4cnc(C)cc4c3C(=O)N2)on1] | 957 |
| 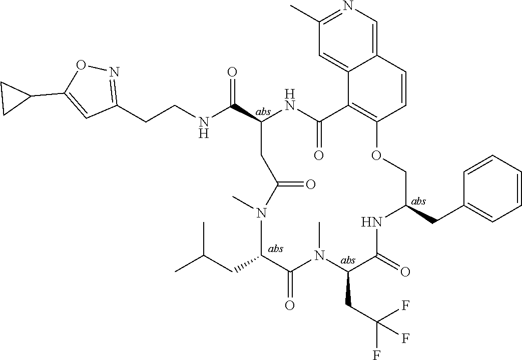 [SMILES: Cc1cc2c3c(ccc2cn1)OC[C@@H](Cc1ccccc1)NC(=O)[C@@H](CC(F)(F)F)N(C)C(=O)[C@H](CC(C)C)N(C)C(=O)C[C@@H](C(=O)NCCc1cc(C2CC2)on1)NC3=O] | 958 |
| 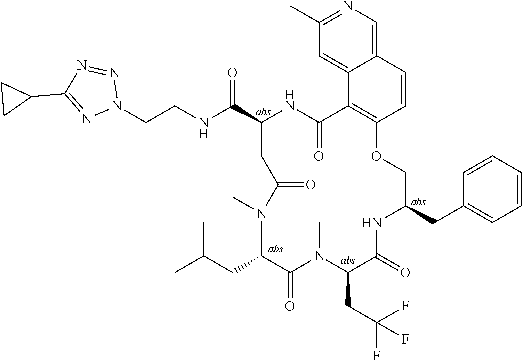 [SMILES: Cc1cc2c3c(ccc2cn1)OC[C@@H](Cc1ccccc1)NC(=O)[C@@H](CC(F)(F)F)N(C)C(=O)[C@H](CC(C)C)N(C)C(=O)C[C@@H](C(=O)NCCn1nnc(C2CC2)n1)NC3=O] | 959 |
| 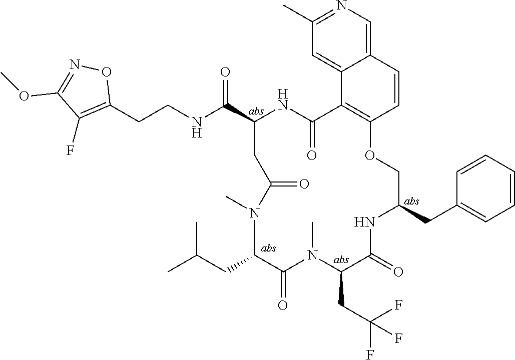 [SMILES: COc1noc(CCNC(=O)[C@@H]2CC(=O)N(C)[C@@H](CC(C)C)C(=O)N(C)[C@H](CC(F)(F)F)C(=O)N[C@H](Cc3ccccc3)COc3ccc4cnc(C)cc4c3C(=O)N2)c1F] | 960 |
| 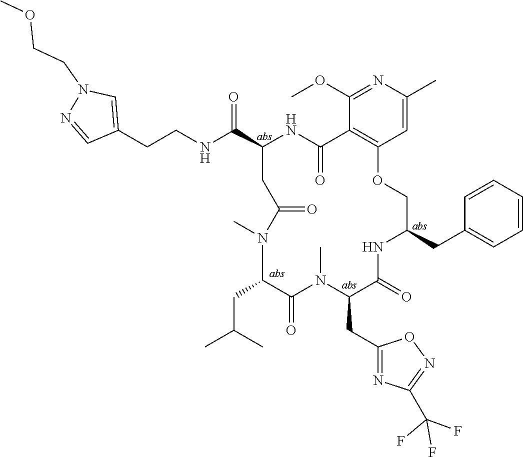 [SMILES: COCCn1cc(CCNC(=O)[C@@H]2CC(=O)N(C)[C@@H](CC(C)C)C(=O)N(C)[C@H](Cc3nc(C(F)(F)F)no3)C(=O)N[C@H](Cc3ccccc3)COc3cc(C)nc(OC)c3C(=O)N2)cn1] | 961 |
| 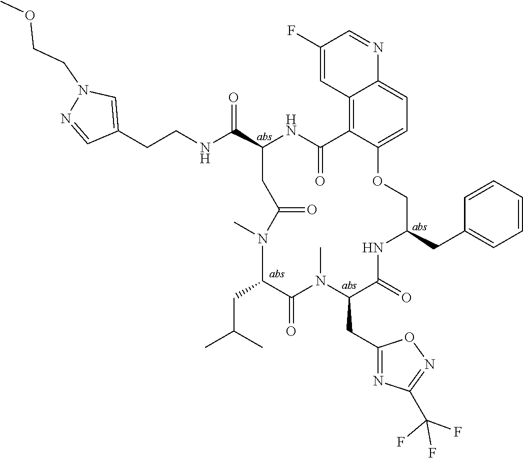 [SMILES: COCCn1cc(CCNC(=O)[C@@H]2CC(=O)N(C)[C@@H](CC(C)C)C(=O)N(C)[C@H](Cc3nc(C(F)(F)F)no3)C(=O)N[C@H](Cc3ccccc3)COc3ccc4ncc(F)cc4c3C(=O)N2)cn1] | 962 |
| 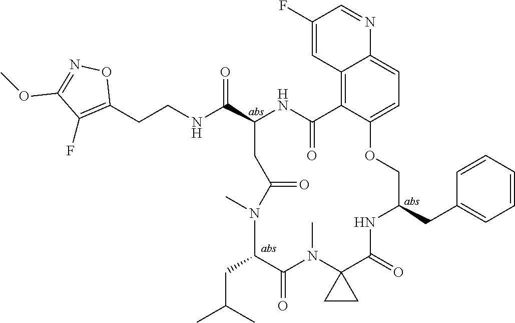 [SMILES: COc1noc(CCNC(=O)[C@@H]2CC(=O)N(C)[C@@H](CC(C)C)C(=O)N(C)C3(CC3)C(=O)N[C@H](Cc3ccccc3)COc3ccc4ncc(F)cc4c3C(=O)N2)c1F] | 963 |
| 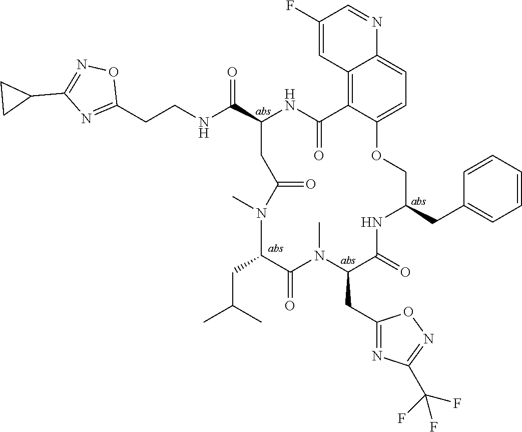 [SMILES: CC(C)C[C@H]1C(=O)N(C)[C@H](Cc2nc(C(F)(F)F)no2)C(=O)N[C@H](Cc2ccccc2)COc2ccc3ncc(F)cc3c2C(=O)N[C@H](C(=O)NCCc2nc(C3CC3)no2)CC(=O)N1C] | 964 |
| 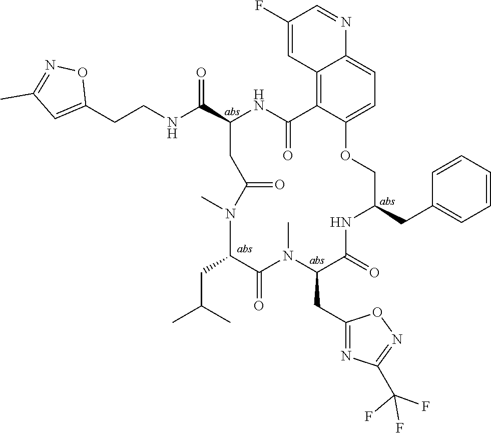 [SMILES: Cc1cc(CCNC(=O)[C@@H]2CC(=O)N(C)[C@@H](CC(C)C)C(=O)N(C)[C@H](Cc3nc(C(F)(F)F)no3)C(=O)N[C@H](Cc3ccccc3)COc3ccc4ncc(F)cc4c3C(=O)N2)on1] | 965 |
| 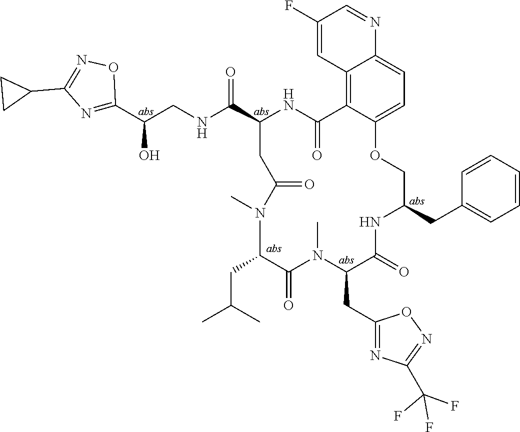 [SMILES: CC(C)C[C@H]1C(=O)N(C)[C@H](Cc2nc(C(F)(F)F)no2)C(=O)N[C@H](Cc2ccccc2)COc2ccc3ncc(F)cc3c2C(=O)N[C@H](C(=O)NC[C@@H](O)c2nc(C3CC3)no2)CC(=O)N1C] | 966 |
| 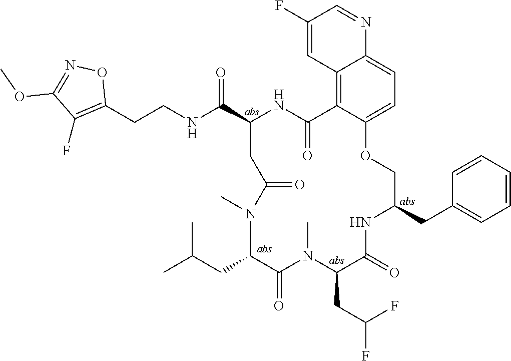 [SMILES: COc1noc(CCNC(=O)[C@@H]2CC(=O)N(C)[C@@H](CC(C)C)C(=O)N(C)[C@H](CC(F)F)C(=O)N[C@H](Cc3ccccc3)COc3ccc4ncc(F)cc4c3C(=O)N2)c1F] | 967 |
| 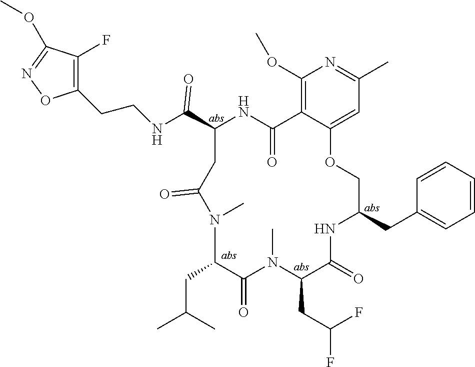 [SMILES: COc1noc(CCNC(=O)[C@@H]2CC(=O)N(C)[C@@H](CC(C)C)C(=O)N(C)[C@H](CC(F)F)C(=O)N[C@H](Cc3ccccc3)COc3cc(C)nc(OC)c3C(=O)N2)c1F] | 968 |
| 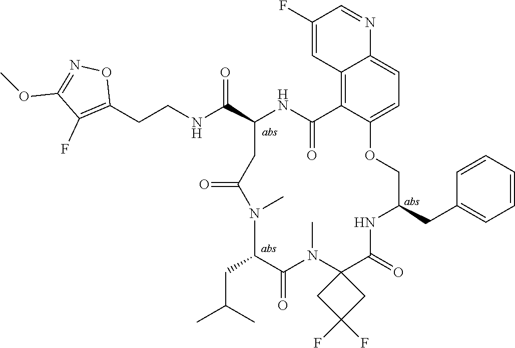 [SMILES: COc1noc(CCNC(=O)[C@@H]2CC(=O)N(C)[C@@H](CC(C)C)C(=O)N(C)C3(CC(F)(F)C3)C(=O)N[C@H](Cc3ccccc3)COc3ccc4ncc(F)cc4c3C(=O)N2)c1F] | 969 |
| 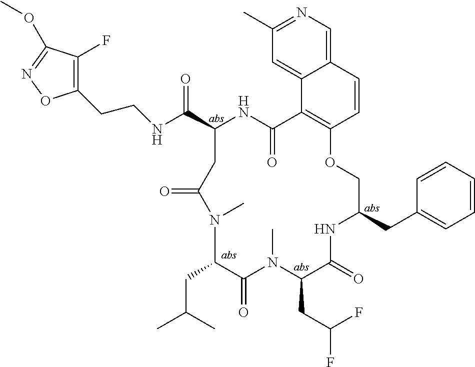 [SMILES: COc1noc(CCNC(=O)[C@@H]2CC(=O)N(C)[C@@H](CC(C)C)C(=O)N(C)[C@H](CC(F)F)C(=O)N[C@H](Cc3ccccc3)COc3ccc4cnc(C)cc4c3C(=O)N2)c1F] | 970 |
| 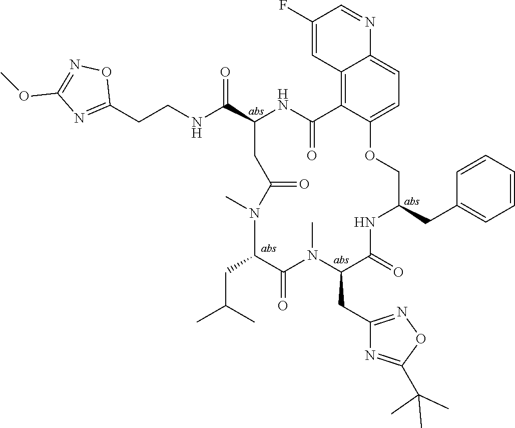 [SMILES: COc1noc(CCNC(=O)[C@@H]2CC(=O)N(C)[C@@H](CC(C)C)C(=O)N(C)[C@H](Cc3noc(C(C)(C)C)n3)C(=O)N[C@H](Cc3ccccc3)COc3ccc4ncc(F)cc4c3C(=O)N2)n1] | 971 |
| 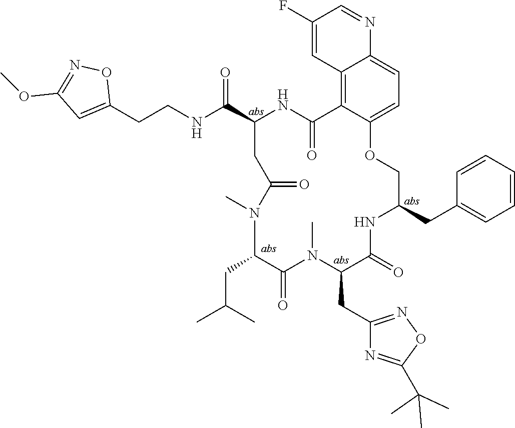 [SMILES: COc1cc(CCNC(=O)[C@@H]2CC(=O)N(C)[C@@H](CC(C)C)C(=O)N(C)[C@H](Cc3noc(C(C)(C)C)n3)C(=O)N[C@H](Cc3ccccc3)COc3ccc4ncc(F)cc4c3C(=O)N2)on1] | 972 |
| 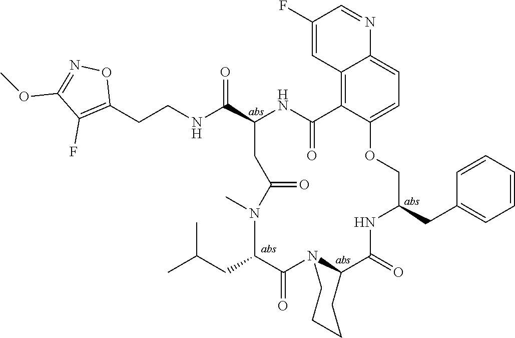 | 973 |
| 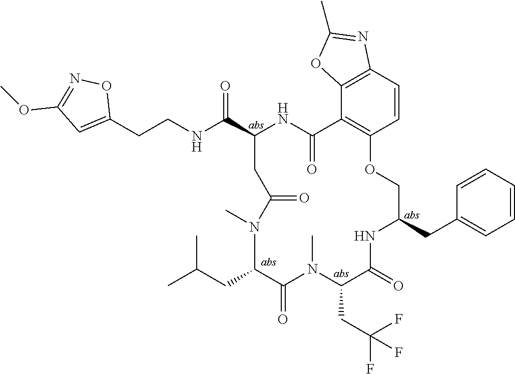 [SMILES: COc1cc(CCNC(=O)[C@@H]2CC(=O)N(C)[C@@H](CC(C)C)C(=O)N(C)[C@@H](CC(F)(F)F)C(=O)N[C@H](Cc3ccccc3)COc3ccc4nc(C)oc4c3C(=O)N2)on1] | 974 |
| 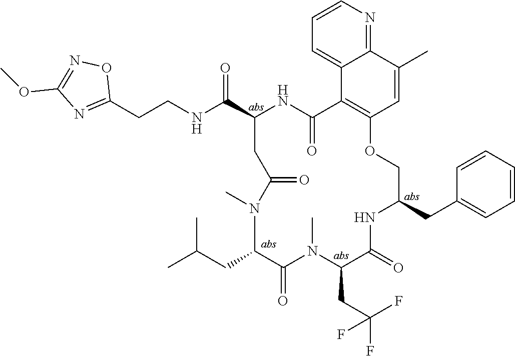 [SMILES: COc1noc(CCNC(=O)[C@@H]2CC(=O)N(C)[C@@H](CC(C)C)C(=O)N(C)[C@H](CC(F)(F)F)C(=O)N[C@H](Cc3ccccc3)COc3cc(C)c4ncccc4c3C(=O)N2)n1] | 975 |
| 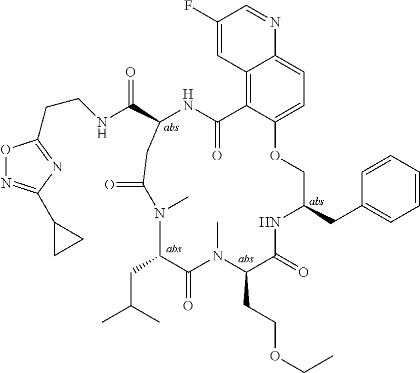 [SMILES: CCOCC[C@@H]1C(=O)N[C@H](Cc2ccccc2)COc2ccc3ncc(F)cc3c2C(=O)N[C@H](C(=O)NCCc2nc(C3CC3)no2)CC(=O)N(C)[C@@H](CC(C)C)C(=O)N1C] | 976 |
| 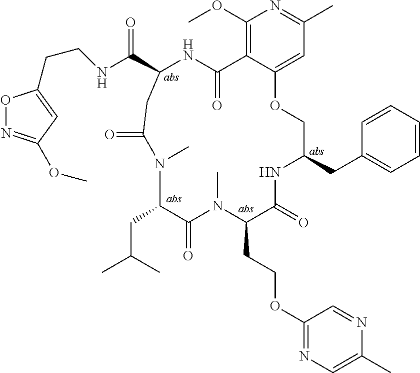 [SMILES: COc1cc(CCNC(=O)[C@@H]2CC(=O)N(C)[C@@H](CC(C)C)C(=O)N(C)[C@H](CCOc3cnc(C)cn3)C(=O)N[C@H](Cc3ccccc3)COc3cc(C)nc(OC)c3C(=O)N2)on1] | 977 |
| 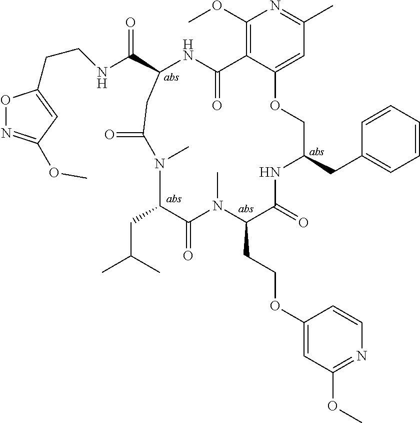 [SMILES: COc1cc(OCC[C@@H]2C(=O)N[C@H](Cc3ccccc3)COc3cc(C)nc(OC)c3C(=O)N[C@H](C(=O)NCCc3cc(OC)no3)CC(=O)N(C)[C@@H](CC(C)C)C(=O)N2C)ccn1] | 978 |
| 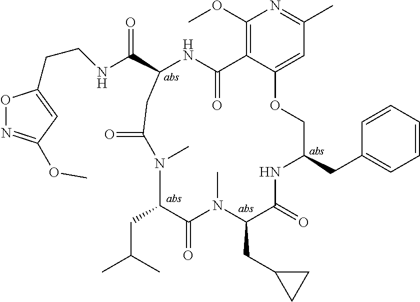 [SMILES: COc1cc(CCNC(=O)[C@@H]2CC(=O)N(C)[C@@H](CC(C)C)C(=O)N(C)[C@H](CC3CC3)C(=O)N[C@H](Cc3ccccc3)COc3cc(C)nc(OC)c3C(=O)N2)on1] | 979 |
| 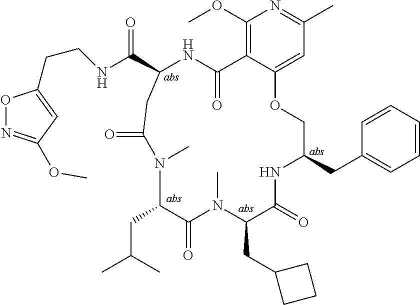 [SMILES: COc1cc(CCNC(=O)[C@@H]2CC(=O)N(C)[C@@H](CC(C)C)C(=O)N(C)[C@H](CC3CCC3)C(=O)N[C@H](Cc3ccccc3)COc3cc(C)nc(OC)c3C(=O)N2)on1] | 980 |
| 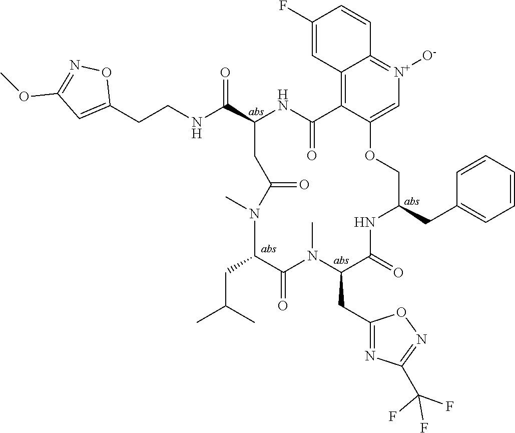 [SMILES: COc1cc(CCNC(=O)[C@@H]2CC(=O)N(C)[C@@H](CC(C)C)C(=O)N(C)[C@H](Cc3nc(C(F)(F)F)no3)C(=O)N[C@H](Cc3ccccc3)COc3c[n+]([O-])c4ccc(F)cc4c3C(=O)N2)on1] | 981 |
| 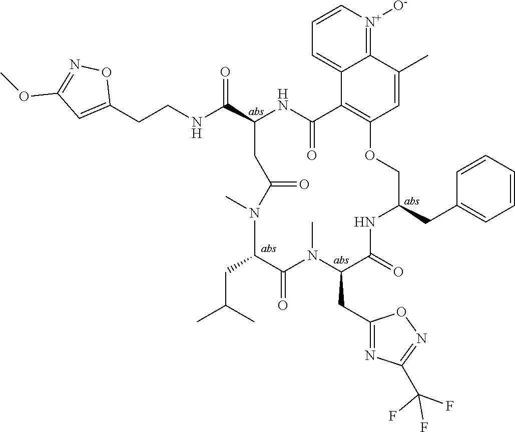 [SMILES: COc1cc(CCNC(=O)[C@@H]2CC(=O)N(C)[C@@H](CC(C)C)C(=O)N(C)[C@H](Cc3nc(C(F)(F)F)no3)C(=O)N[C@H](Cc3ccccc3)COc3cc(C)c4c(ccc[n+]4[O-])c3C(=O)N2)on1] | 982 |
| 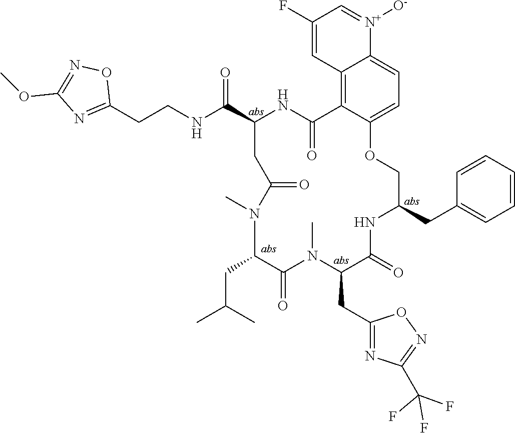 [SMILES: COc1noc(CCNC(=O)[C@@H]2CC(=O)N(C)[C@@H](CC(C)C)C(=O)N(C)[C@H](Cc3nc(C(F)(F)F)no3)C(=O)N[C@H](Cc3ccccc3)COc3ccc4c(cc(F)c[n+]4[O-])c3C(=O)N2)n1] | 983 |

In Tables 3, 4 and 5 above, a stereogenic or asymmetric center indicated in the structures as “abs” represents said stereogenic or asymmetric center in the respective enantiomerically enriched absolute (R)- or (S)-configuration as depicted. A stereogenic or asymmetric center indicated in the structures as “&1” represents said stereogenic or asymmetric center in the respective (RS)-configuration, i.e. comprising the respective enantiomerically enriched (R)-configuration, or enantiomerically enriched (S)-configuration, or any mixture of epimers at such center.

### II. Biological Assays

Compounds of the present invention may be further characterized with regard to their general pharmacokinetic and pharmacological properties using conventional assays well known in the art for example relating to their bioavailablility in different species (such as rat or dog); or for their properties with regard to drug safety and/or toxicological properties using conventional assays well known in the art, for example relating to cytochrome P450 enzyme inhibition and time dependent inhibition, pregnane X receptor (PXR) activation, glutathione binding, or phototoxic behavior.

Biological in vitro Assays

Evaluation of Compound EC₅₀ and Eₘₐₓ Values

The corrector activities of the compounds of formula (I) on CFTR are determined in accordance with the following experimental method. The method measures the effect of over-night compound incubation on F508del-CFTR cell surface expression in a recombinant U2OS cell line (DiscoveRx, #93-0987C3). This cell line is engineered to co-express (i) human F508del-CFTR tagged with a Prolink (PK=short ß-galactosidase fragment) and (ii) the remainder of the ß-galactosidase enzyme (Enzyme Acceptor; EA) localized to the plasma membrane. Incubation with compounds that increase PK-tagged F508del-CFTR at the plasma membrane will lead to complementation of the EA fragment to form a functional ß-galactosidase enzyme which is quantified by a chemiluminescence reaction.

Briefly, the cells are seeded at 3500cells/well into 384-well low volume plates (Corning, #3826) in 20 μl of full medium (Mc Coy's 5a (#36600-021, Gibco)+10% FBS Gibco+penicillin/streptomycin). The cells are incubated for 5h in the incubator before the addition of 5 μl/well of compound dilution series (5× working stocks in full medium). Final DMSO concentration in the assay is 0.25%. The cells are co-incubated with the compounds for 16h in the incubator at 37° C., 5% CO₂. The next day, the cell plates are incubated for 2h at RT in the dark. Then, 10 μl/well of Flash detection reagent (DiscoverX, #93-0247) is added, the plate is incubated for another 30 min at RT in the dark and chemiluminescence is measured. Concentration-response curves are generated using compound-intrinsic maximal efficacy as upper plateau, and from these CRCs compound-intrinsic EC₅₀ values are determined. Compound-specific Eₘₐₓ values are calculated in relation to the Eₘₐₓ of the corrector lumacaftor (Eₘₐₓlumacaftor=100%).

The calculated EC₅₀ values may fluctuate depending on the daily assay performance. Fluctuations of this kind are known to those skilled in the art. EC₅₀ values from several measurements are given as geomean values. The calculated Eₘₐₓ values may fluctuate depending on the daily assay performance. Fluctuations of this kind are known to those skilled in the art. Eₘₐₓ values from several measurements are given as arithmetic mean values.

### REFERENCES

Elborn J S. (2016) Cystic fibrosis. Lancet 388:2519-2531. Dalemans W, Barbry P, Champigny G, Jallat S, Dott K, Dreyer D, Crystal R G, Pavirani A, Lecocq J P, Lazdunski M (1991) Altered chloride ion channel kinetics associated with the delta F508 cystic fibrosis mutation. Nature 354: 526-8.

### TABLE OF BIOLOGICAL DATA:

| | EC₅₀ | Emax |
|---|---|---|
| Ex. | [nmol/l] | [%] |
| 1 | 56 | 105 |
| 2 | 110 | 150 |
| 3 | 816 | 151 |
| 4 | 673 | 195 |
| 5 | 369 | 109 |
| 6 | 420 | 112 |
| 7 | 398 | 118 |
| 8 | 887 | 210 |
| 9 | 360 | 130 |
| 10 | 82 | 123 |
| 11 | 182 | 132 |
| 12 | 270 | 184 |
| 13 | 268 | 153 |
| 14 | 160 | 185 |
| 15 | 187 | 240 |
| 16 | 269 | 137 |
| 17 | 466 | 180 |
| 18 | 388 | 117 |
| 19 | 743 | 125 |
| 20 | 721 | 102 |
| 21 | 350 | 108 |
| 22 | 600 | 104 |
| 23 | 991 | 115 |
| 24 | 823 | 131 |
| 25 | 547 | 142 |
| 26 | 299 | 111 |
| 27 | 474 | 114 |
| 28 | 603 | 106 |
| 29 | 439 | 140 |
| 30 | 275 | 113 |
| 31 | 463 | 111 |
| 32 | 318 | 146 |
| 33 | 255 | 141 |
| 34 | 260 | 121 |
| 35 | 262 | 109 |
| 36 | 306 | 120 |
| 37 | 122 | 205 |
| 38 | 299 | 191 |
| 39 | 415 | 450 |
| 40 | 897 | 147 |
| 41 | 280 | 215 |
| 42 | 353 | 419 |
| 43 | 862 | 137 |
| 44 | 571 | 247 |
| 45 | 682 | 324 |
| 46 | 679 | 137 |
| 47 | 213 | 247 |
| 48 | 409 | 151 |
| 49 | 347 | 136 |
| 50 | 558 | 256 |
| 51 | 352 | 187 |
| 52 | 348 | 176 |
| 53 | 596 | 404 |
| 54 | 558 | 373 |
| 55 | 945 | 245 |
| 56 | 764 | 256 |
| 57 | 414 | 315 |
| 58 | 759 | 175 |
| 59 | 772 | 237 |
| 60 | 662 | 228 |
| 61 | 421 | 202 |
| 62 | 809 | 222 |
| 63 | 418 | 250 |
| 64 | 441 | 143 |
| 65 | 647 | 282 |
| 66 | 587 | 169 |
| 67 | 615 | 236 |
| 68 | 448 | 331 |
| 69 | 454 | 262 |
| 70 | 754 | 202 |
| 71 | 338 | 326 |
| 72 | 142 | 335 |
| 73 | 214 | 253 |
| 74 | 128 | 271 |
| 75 | 255 | 191 |
| 76 | 196 | 283 |
| 77 | 231 | 237 |
| 78 | 544 | 277 |
| 79 | 327 | 296 |
| 80 | 170 | 308 |
| 81 | 233 | 232 |
| 82 | 598 | 246 |
| 83 | 731 | 309 |
| 84 | 274 | 299 |
| 85 | 260 | 244 |
| 86 | 314 | 157 |
| 87 | 162 | 190 |
| 88 | 230 | 112 |
| 89 | 132 | 262 |
| 90 | 364 | 129 |
| 91 | 466 | 210 |
| 92 | 289 | 256 |
| 93 | 777 | 189 |
| 94 | 257 | 201 |
| 95 | 250 | 178 |
| 96 | 425 | 206 |
| 97 | 146 | 285 |
| 98 | 311 | 326 |
| 99 | 205 | 254 |
| 100 | 184 | 273 |
| 101 | 201 | 163 |
| 102 | 225 | 332 |
| 103 | 499 | 256 |
| 104 | 211 | 204 |
| 105 | 173 | 351 |
| 106 | 864 | 308 |
| 107 | 794 | 257 |
| 108 | 581 | 222 |
| 109 | 231 | 321 |
| 110 | 478 | 261 |
| 111 | 392 | 242 |
| 112 | 817 | 200 |
| 113 | 369 | 158 |
| 114 | 924 | 220 |
| 115 | 380 | 250 |
| 116 | 554 | 253 |
| 117 | 776 | 237 |
| 118 | 329 | 265 |
| 119 | 328 | 248 |
| 120 | 331 | 212 |
| 121 | 615 | 354 |
| 122 | 324 | 292 |
| 123 | 873 | 280 |
| 124 | 382 | 347 |
| 125 | 271 | 229 |
| 126 | 261 | 230 |
| 127 | 107 | 311 |
| 128 | 190 | 292 |
| 129 | 406 | 264 |
| 130 | 1000 | 264 |
| 131 | 127 | 320 |
| 132 | 110 | 313 |
| 133 | 149 | 213 |
| 134 | 506 | 150 |
| 135 | 177 | 255 |
| 136 | 270 | 271 |
| 137 | 254 | 311 |
| 138 | 538 | 292 |
| 139 | 463 | 381 |
| 140 | 142 | 318 |
| 141 | 313 | 331 |
| 142 | 194 | 381 |
| 143 | 922 | 320 |
| 144 | 211 | 327 |
| 145 | 309 | 107 |
| 146 | 247 | 318 |
| 147 | 270 | 339 |
| 148 | 811 | 198 |
| 149 | 191 | 259 |
| 150 | 151 | 130 |
| 151 | 759 | 106 |
| 152 | 668 | 133 |
| 153 | 460 | 115 |
| 154 | 398 | 102 |
| 155 | 260 | 144 |
| 156 | 647 | 123 |
| 157 | 395 | 121 |
| 158 | 315 | 169 |
| 159 | 385 | 100 |
| 160 | 224 | 116 |
| 161 | 250 | 137 |
| 162 | 472 | 132 |
| 163 | 377 | 185 |
| 164 | 519 | 167 |
| 165 | 287 | 149 |
| 166 | 624 | 111 |
| 167 | 822 | 238 |
| 168 | 338 | 193 |
| 169 | 334 | 170 |
| 170 | 329 | 267 |
| 171 | 144 | 301 |
| 172 | 324 | 213 |
| 173 | 455 | 212 |
| 174 | 954 | 194 |
| 175 | 229 | 179 |
| 176 | 821 | 245 |
| 177 | 647 | 248 |
| 178 | 680 | 237 |
| 179 | 731 | 251 |
| 180 | 388 | 257 |
| 181 | 718 | 251 |
| 182 | 406 | 270 |
| 183 | 621 | 237 |
| 184 | 410 | 205 |
| 185 | 379 | 275 |
| 186 | 644 | 209 |
| 187 | 645 | 267 |
| 188 | 586 | 275 |
| 189 | 453 | 270 |
| 190 | 334 | 310 |
| 191 | 474 | 295 |
| 192 | 316 | 107 |
| 193 | 521 | 118 |
| 194 | 613 | 178 |
| 195 | 889 | 202 |
| 196 | 425 | 106 |
| 197 | 579 | 132 |
| 198 | 195 | 243 |
| 199 | 230 | 214 |
| 200 | 210 | 270 |
| 201 | 222 | 109 |
| 202 | 187 | 160 |
| 203 | 183 | 134 |
| 204 | 198 | 173 |
| 205 | 175 | 229 |
| 206 | 272 | 164 |
| 207 | 317 | 138 |
| 208 | 277 | 188 |
| 209 | 392 | 118 |
| 210 | 352 | 108 |
| 211 | 309 | 146 |
| 212 | 292 | 130 |
| 213 | 292 | 100 |
| 214 | 292 | 221 |
| 215 | 214 | 172 |
| 216 | 420 | 102 |
| 217 | 202 | 135 |
| 218 | 353 | 143 |
| 219 | 206 | 182 |
| 220 | 258 | 103 |
| 221 | 833 | 229 |
| 222 | 216 | 249 |
| 223 | 388 | 141 |
| 224 | 340 | 144 |
| 225 | 191 | 110 |
| 226 | 520 | 213 |
| 227 | 290 | 103 |
| 228 | 460 | 190 |
| 229 | 279 | 117 |
| 230 | 482 | 103 |
| 231 | 592 | 173 |
| 232 | 526 | 105 |
| 233 | 610 | 92 |
| 234 | 471 | 110 |
| 235 | 825 | 117 |
| 236 | 435 | 126 |
| 237 | 300 | 128 |
| 238 | 375 | 153 |
| 239 | 514 | 142 |
| 240 | 1260 | 77 |
| 241 | 1660 | 92 |
| 242 | 177 | 232 |
| 243 | 181 | 232 |
| 244 | 177 | 239 |
| 245 | 260 | 174 |
| 246 | 140 | 105 |
| 247 | 146 | 111 |
| 248 | 106 | 191 |
| 249 | 116 | 175 |
| 250 | 86 | 160 |
| 251 | 83 | 159 |
| 252 | 888 | 111 |
| 253 | 88 | 136 |
| 254 | 102 | 152 |
| 255 | 183 | 299 |
| 256 | 147 | 319 |
| 257 | 249 | 116 |
| 258 | 424 | 117 |
| 259 | 428 | 136 |
| 260 | 783 | 243 |
| 261 | 327 | 215 |
| 262 | 339 | 231 |
| 263 | 448 | 140 |
| 264 | 442 | 117 |
| 265 | 266 | 106 |
| 266 | 357 | 136 |
| 267 | 107 | 275 |
| 268 | 100 | 273 |
| 269 | 350 | 251 |
| 270 | 423 | 172 |
| 271 | 266 | 185 |
| 272 | 374 | 176 |
| 273 | 365 | 169 |
| 274 | 288 | 186 |
| 275 | 148 | 333 |
| 276 | 91 | 333 |
| 277 | 830 | 134 |
| 278 | 709 | 167 |
| 279 | 643 | 112 |
| 280 | 297 | 188 |
| 281 | 110 | 361 |
| 282 | 114 | 360 |
| 283 | 272 | 231 |
| 284 | 337 | 136 |
| 285 | 239 | 118 |
| 286 | 177 | 144 |
| 287 | 207 | 154 |
| 288 | 147 | 175 |
| 289 | 186 | 160 |
| 290 | 188 | 260 |
| 291 | 109 | 210 |
| 292 | 114 | 191 |
| 293 | 340 | 137 |
| 294 | 265 | 138 |
| 295 | 202 | 174 |
| 296 | 158 | 144 |
| 297 | 193 | 225 |
| 298 | 220 | 186 |
| 299 | 109 | 136 |
| 300 | 103 | 210 |
| 301 | 112 | 202 |
| 302 | 106 | 193 |
| 303 | 180 | 275 |
| 304 | 581 | 193 |
| 305 | 533 | 123 |
| 306 | 923 | 139 |
| 307 | 423 | 113 |
| 308 | 469 | 103 |
| 309 | 456 | 113 |
| 310 | 788 | 202 |
| 311 | 454 | 127 |
| 312 | 727 | 111 |
| 313 | 313 | 119 |
| 314 | 469 | 111 |
| 315 | 115 | 349 |
| 316 | 55 | 343 |
| 317 | 661 | 89 |
| 318 | 679 | 295 |
| 319 | 574 | 296 |
| 320 | 363 | 297 |
| 321 | 816 | 266 |
| 322 | 504 | 197 |
| 323 | 742 | 258 |
| 324 | 666 | 247 |
| 325 | 187 | 237 |
| 326 | 276 | 273 |
| 327 | 461 | 225 |
| 328 | 987 | 228 |
| 329 | 796 | 198 |
| 330 | 418 | 138 |
| 331 | 121 | 240 |
| 332 | 309 | 183 |
| 333 | 345 | 182 |
| 334 | 323 | 232 |
| 335 | 270 | 262 |
| 336 | 207 | 212 |
| 337 | 452 | 116 |
| 338 | 292 | 173 |
| 339 | 195 | 237 |
| 340 | 278 | 139 |
| 341 | 144 | 237 |
| 342 | 464 | 128 |
| 343 | 332 | 270 |
| 344 | 126 | 284 |
| 345 | 95 | 268 |
| 346 | 106 | 282 |
| 347 | 131 | 282 |
| 348 | 340 | 155 |
| 349 | 597 | 258 |
| 350 | 546 | 299 |
| 351 | 756 | 127 |
| 352 | 297 | 262 |
| 353 | 513 | 177 |
| 354 | 231 | 321 |
| 355 | 87 | 307 |
| 356 | 167 | 353 |
| 357 | 475 | 124 |
| 358 | 411 | 171 |
| 359 | 318 | 144 |
| 360 | 184 | 224 |
| 361 | 264 | 183 |
| 362 | 153 | 247 |
| 363 | 252 | 230 |
| 364 | 264 | 201 |
| 365 | 398 | 387 |
| 366 | 265 | 172 |
| 367 | 249 | 321 |
| 368 | 918 | 147 |
| 369 | 107 | 243 |
| 370 | 388 | 229 |
| 371 | 226 | 180 |
| 372 | 72 | 360 |
| 373 | 200 | 149 |
| 374 | 102 | 333 |
| 375 | 461 | 316 |
| 376 | 156 | 284 |
| 377 | 704 | 111 |
| 378 | 45 | 138 |
| 379 | 76 | 312 |
| 380 | 65 | 325 |
| 381 | 68 | 322 |
| 382 | 44 | 264 |
| 383 | 65 | 317 |
| 384 | 54 | 282 |
| 385 | 191 | 326 |
| 386 | 393 | 149 |
| 387 | 342 | 215 |
| 388 | 310 | 163 |
| 389 | 115 | 320 |
| 390 | 269 | 188 |
| 391 | 70 | 319 |
| 392 | 327 | 144 |
| 393 | 118 | 314 |
| 394 | 262 | 151 |
| 395 | 110 | 316 |
| 396 | 241 | 190 |
| 397 | 262 | 207 |
| 398 | 125 | 280 |
| 399 | 506 | 146 |
| 400 | 164 | 292 |
| 401 | 559 | 189 |
| 402 | 305 | 314 |
| 403 | 550 | 179 |
| 404 | 157 | 254 |
| 405 | 343 | 171 |
| 406 | 409 | 173 |
| 407 | 684 | 123 |
| 408 | 241 | 134 |
| 409 | 214 | 144 |
| 410 | 129 | 269 |
| 411 | 138 | 250 |
| 412 | 377 | 291 |
| 413 | 557 | 129 |
| 414 | 372 | 251 |
| 415 | 840 | 274 |
| 416 | 526 | 345 |
| 417 | 437 | 343 |
| 418 | 276 | 259 |
| 419 | 358 | 243 |
| 420 | 437 | 231 |
| 421 | 391 | 320 |
| 422 | 192 | 193 |
| 423 | 278 | 239 |
| 424 | 831 | 235 |
| 425 | 348 | 364 |
| 426 | 269 | 217 |
| 427 | 248 | 274 |
| 428 | 614 | 218 |
| 429 | 606 | 224 |
| 430 | 964 | 194 |
| 431 | 208 | 281 |
| 432 | 474 | 413 |
| 433 | 167 | 344 |
| 434 | 664 | 327 |
| 435 | 895 | 119 |
| 436 | 458 | 300 |
| 437 | 844 | 159 |
| 438 | 474 | 422 |
| 439 | 253 | 345 |
| 440 | 788 | 296 |
| 441 | 815 | 326 |
| 442 | 443 | 154 |
| 443 | 316 | 275 |
| 444 | 1050 | 199 |
| 445 | 958 | 194 |
| 446 | 900 | 248 |
| 447 | 447 | 220 |
| 448 | 961 | 109 |
| 449 | 543 | 165 |
| 450 | 379 | 107 |
| 451 | 257 | 307 |
| 452 | 886 | 292 |
| 453 | 417 | 251 |
| 454 | 234 | 236 |
| 455 | 677 | 211 |
| 456 | 250 | 185 |
| 457 | 326 | 144 |
| 458 | 209 | 238 |
| 459 | 726 | 390 |
| 460 | 310 | 398 |
| 461 | 444 | 262 |
| 462 | 345 | 130 |
| 463 | 278 | 269 |
| 464 | 319 | 109 |
| 465 | 800 | 387 |
| 466 | 548 | 328 |
| 467 | 936 | 152 |
| 468 | 504 | 361 |
| 469 | 381 | 110 |
| 470 | 662 | 169 |
| 471 | 981 | 278 |
| 472 | 284 | 239 |
| 473 | 585 | 119 |
| 474 | 478 | 359 |
| 475 | 992 | 314 |
| 476 | 333 | 344 |
| 477 | 754 | 327 |
| 478 | 168 | 337 |
| 479 | 480 | 253 |
| 480 | 201 | 276 |
| 481 | 448 | 225 |
| 482 | 414 | 247 |
| 483 | 517 | 311 |
| 484 | 251 | 117 |
| 485 | 238 | 204 |
| 486 | 201 | 303 |
| 487 | 1340 | 147 |
| 488 | 910 | 270 |
| 489 | 625 | 273 |
| 490 | 222 | 181 |
| 491 | 209 | 178 |
| 492 | 305 | 168 |
| 493 | 687 | 253 |
| 494 | 260 | 295 |
| 495 | 296 | 207 |
| 496 | 523 | 317 |
| 497 | 535 | 171 |
| 498 | 198 | 313 |
| 499 | 189 | 321 |
| 500 | 553 | 199 |
| 501 | 198 | 308 |
| 502 | 752 | 295 |
| 503 | 772 | 137 |
| 504 | 149 | 298 |
| 505 | 229 | 330 |
| 506 | 318 | 211 |
| 507 | 469 | 337 |
| 508 | 382 | 378 |
| 509 | 75 | 327 |
| 510 | 108 | 256 |
| 511 | 624 | 314 |
| 512 | 300 | 316 |
| 513 | 815 | 169 |
| 514 | 170 | 304 |
| 515 | 518 | 140 |
| 516 | 494 | 155 |
| 517 | 986 | 222 |
| 518 | 655 | 189 |
| 519 | 184 | 332 |
| 520 | 345 | 155 |
| 521 | 433 | 191 |
| 522 | 204 | 237 |
| 523 | 483 | 352 |
| 524 | 60 | 339 |
| 525 | 56 | 288 |
| 526 | 394 | 167 |
| 527 | 151 | 318 |
| 528 | 321 | 337 |
| 529 | 947 | 206 |
| 530 | 334 | 353 |
| 531 | 265 | 336 |
| 532 | 397 | 375 |
| 533 | 221 | 352 |
| 534 | 273 | 394 |
| 535 | 407 | 424 |
| 536 | 175 | 358 |
| 537 | 384 | 294 |
| 538 | 80 | 340 |
| 539 | 95 | 391 |
| 540 | 269 | 256 |
| 541 | 185 | 357 |
| 542 | 368 | 334 |
| 543 | 785 | 399 |
| 544 | 774 | 398 |
| 545 | 349 | 385 |
| 546 | 350 | 411 |
| 547 | 281 | 330 |
| 548 | 106 | 326 |
| 549 | 166 | 167 |
| 550 | 687 | 129 |
| 551 | 222 | 317 |
| 552 | 413 | 358 |
| 553 | 287 | 304 |
| 554 | 324 | 310 |
| 555 | 219 | 309 |
| 556 | 327 | 322 |
| 557 | 594 | 253 |
| 558 | 167 | 355 |
| 559 | 155 | 236 |
| 560 | 114 | 249 |
| 561 | 437 | 215 |
| 562 | 444 | 282 |
| 563 | 125 | 330 |
| 564 | 49 | 298 |
| 565 | 338 | 254 |
| 566 | 703 | 278 |
| 567 | 384 | 307 |
| 568 | 391 | 238 |
| 569 | 108 | 270 |
| 570 | 374 | 323 |
| 571 | 47 | 347 |
| 572 | 701 | 200 |
| 573 | 423 | 306 |
| 574 | 308 | 319 |
| 575 | 411 | 273 |
| 576 | 523 | 268 |
| 577 | 600 | 268 |
| 578 | 53 | 319 |
| 579 | 399 | 131 |
| 580 | 198 | 319 |
| 581 | 300 | 303 |
| 582 | 116 | 279 |
| 583 | 339 | 90 |
| 584 | 575 | 156 |
| 585 | 176 | 168 |
| 586 | 221 | 165 |
| 587 | 414 | 106 |
| 588 | 650 | 102 |
| 589 | 298 | 160 |
| 590 | 362 | 201 |
| 591 | 341 | 259 |
| 592 | 881 | 119 |
| 593 | 614 | 190 |
| 594 | 810 | 171 |
| 595 | 332 | 233 |
| 596 | 167 | 195 |
| 597 | 168 | 211 |
| 598 | 759 | 312 |
| 599 | 151 | 299 |
| 600 | 97 | 286 |
| 601 | 589 | 138 |
| 602 | 286 | 299 |
| 603 | 205 | 301 |
| 604 | 354 | 103 |
| 605 | 637 | 257 |
| 606 | 521 | 150 |
| 607 | 121 | 283 |
| 608 | 804 | 267 |
| 609 | 574 | 320 |
| 610 | 89 | 311 |
| 611 | 344 | 251 |
| 612 | 148 | 298 |
| 613 | 82 | 312 |
| 614 | 484 | 317 |
| 615 | 465 | 170 |
| 616 | 581 | 92 |
| 617 | 75 | 318 |
| 618 | 80 | 309 |
| 619 | 155 | 322 |
| 620 | 316 | 266 |
| 621 | 339 | 288 |
| 622 | 199 | 296 |
| 623 | 248 | 349 |
| 624 | 921 | 171 |
| 625 | 781 | 186 |
| 626 | 163 | 253 |
| 627 | 786 | 395 |
| 628 | 363 | 355 |
| 629 | 521 | 215 |
| 630 | 261 | 175 |
| 631 | 898 | 133 |
| 632 | 927 | 326 |
| 633 | 652 | 316 |
| 634 | 173 | 178 |
| 635 | 404 | 112 |
| 636 | 91 | 276 |
| 637 | 106 | 300 |
| 638 | 182 | 297 |
| 639 | 173 | 305 |
| 640 | 132 | 339 |
| 641 | 87 | 305 |
| 642 | 168 | 366 |
| 643 | 220 | 270 |
| 644 | 277 | 270 |
| 645 | 289 | 211 |
| 646 | 158 | 201 |
| 647 | 820 | 182 |
| 648 | 748 | 159 |
| 649 | 908 | 149 |
| 650 | 596 | 214 |
| 651 | 375 | 127 |
| 652 | 593 | 155 |
| 653 | 332 | 140 |
| 654 | 290 | 330 |
| 655 | 150 | 318 |
| 656 | 357 | 327 |
| 657 | 347 | 291 |
| 658 | 283 | 300 |
| 659 | 359 | 325 |
| 660 | 91 | 358 |
| 661 | 421 | 247 |
| 662 | 124 | 297 |
| 663 | 801 | 210 |
| 664 | 231 | 291 |
| 665 | 119 | 323 |
| 666 | 484 | 311 |
| 667 | 764 | 149 |
| 668 | 160 | 330 |
| 669 | 856 | 230 |
| 670 | 353 | 167 |
| 671 | 235 | 179 |
| 672 | 103 | 312 |
| 673 | 67 | 314 |
| 674 | 262 | 133 |
| 675 | 194 | 159 |
| 676 | 62 | 267 |
| 677 | 37 | 283 |
| 678 | 355 | 296 |
| 679 | 869 | 277 |
| 680 | 448 | 340 |
| 681 | 447 | 313 |
| 682 | 110 | 351 |
| 683 | 362 | 262 |
| 684 | 986 | 235 |
| 685 | 500 | 234 |
| 686 | 436 | 332 |
| 687 | 794 | 107 |
| 688 | 458 | 104 |
| 689 | 245 | 390 |
| 690 | 478 | 401 |
| 691 | 201 | 364 |
| 692 | 104 | 301 |
| 693 | 40 | 401 |
| 694 | 241 | 213 |
| 695 | 201 | 237 |
| 696 | 14 | 252 |
| 697 | 27 | 306 |
| 698 | 199 | 313 |
| 699 | 299 | 338 |
| 700 | 188 | 186 |
| 701 | 165 | 268 |
| 702 | 295 | 162 |
| 703 | 316 | 167 |
| 704 | 398 | 156 |
| 705 | 305 | 193 |
| 706 | 707 | 162 |
| 707 | 502 | 305 |
| 708 | 874 | 195 |
| 709 | 47 | 284 |
| 710 | 149 | 280 |
| 711 | 778 | 301 |
| 712 | 187 | 211 |
| 713 | 31 | 432 |
| 714 | 36 | 410 |
| 715 | 64 | 283 |
| 716 | 144 | 313 |
| 717 | 69 | 348 |
| 718 | 359 | 272 |
| 719 | 124 | 313 |
| 720 | 881 | 24 |
| 721 | 142 | 328 |
| 722 | 101 | 347 |
| 723 | 512 | 160 |
| 724 | 93 | 345 |
| 725 | 1370 | 200 |
| 726 | 624 | 234 |
| 727 | 5050 | 138 |
| 728 | 235 | 232 |
| 729 | 100 | 258 |
| 730 | 593 | 248 |
| 731 | 381 | 248 |
| 732 | 64 | 239 |
| 733 | 349 | 235 |
| 734 | 299 | 325 |
| 735 | 314 | 176 |
| 736 | 153 | 286 |
| 737 | 665 | 298 |
| 738 | 110 | 256 |
| 739 | 101 | 297 |
| 740 | 111 | 280 |
| 741 | 116 | 294 |
| 742 | 214 | 246 |
| 743 | 207 | 217 |
| 744 | 178 | 239 |
| 745 | 693 | 206 |
| 746 | 211 | 213 |
| 747 | 234 | 213 |
| 748 | 529 | 295 |
| 749 | 153 | 301 |
| 750 | 784 | 267 |
| 751 | 217 | 213 |
| 752 | 706 | 200 |
| 753 | 430 | 264 |
| 754 | 219 | 279 |
| 755 | 168 | 270 |
| 756 | 311 | 252 |
| 757 | 145 | 280 |
| 758 | 334 | 266 |
| 759 | 296 | 131 |
| 760 | 723 | 333 |
| 761 | 676 | 202 |
| 762 | 929 | 333 |
| 763 | 264 | 181 |
| 764 | 374 | 315 |
| 765 | 300 | 323 |
| 766 | 154 | 393 |
| 767 | 212 | 390 |
| 768 | 497 | 292 |
| 769 | 140 | 346 |
| 770 | 1001 | 228 |
| 771 | 597 | 299 |
| 772 | 514 | 343 |
| 773 | 841 | 339 |
| 774 | 751 | 345 |
| 775 | 875 | 359 |
| 776 | 325 | 360 |
| 777 | 875 | 216 |
| 778 | 141 | 302 |
| 779 | 811 | 213 |
| 780 | 116 | 280 |
| 781 | 124 | 296 |
| 782 | 859 | 439 |
| 783 | 889 | 410 |
| 784 | 311 | 391 |
| 785 | 210 | 365 |
| 786 | 156 | 337 |
| 787 | 190 | 323 |
| 788 | 549 | 344 |
| 789 | 675 | 358 |
| 790 | 650 | 350 |
| 791 | 80 | 345 |
| 792 | 628 | 261 |
| 793 | 235 | 105 |
| 794 | 83 | 354 |
| 795 | 141 | 384 |
| 796 | 156 | 321 |
| 797 | 399 | 308 |
| 798 | 197 | 324 |
| 799 | 258 | 335 |
| 800 | 266 | 352 |
| 801 | 149 | 330 |
| 802 | 723 | 200 |
| 803 | 248 | 355 |
| 804 | 115 | 360 |
| 805 | 191 | 276 |
| 806 | 233 | 394 |
| 807 | 206 | 358 |
| 808 | 116 | 399 |
| 809 | 10 | 391 |
| 810 | 161 | 285 |
| 811 | 19 | 333 |
| 812 | 127 | 280 |
| 813 | 110 | 346 |
| 814 | 631 | 242 |
| 815 | 128 | 413 |
| 816 | 741 | 286 |
| 817 | 420 | 375 |
| 818 | 385 | 359 |
| 819 | 276 | 431 |
| 820 | 49 | 461 |
| 821 | 200 | 392 |
| 822 | 131 | 363 |
| 823 | 241 | 366 |
| 824 | 674 | 354 |
| 825 | 353 | 388 |
| 826 | 283 | 340 |
| 827 | 54 | 379 |
| 828 | 299 | 317 |
| 829 | 142 | 320 |
| 830 | 1040 | 310 |
| 831 | 491 | 231 |
| 832 | 518 | 367 |
| 833 | 615 | 411 |
| 834 | 240 | 300 |
| 835 | 363 | 326 |
| 836 | 967 | 257 |
| 837 | 198 | 358 |
| 838 | 118 | 358 |
| 839 | 123 | 336 |
| 840 | 128 | 363 |
| 841 | 120 | 271 |
| 842 | 495 | 305 |
| 843 | 114 | 368 |
| 844 | 1040 | 356 |
| 845 | 453 | 308 |
| 846 | 165 | 261 |
| 847 | 186 | 340 |
| 848 | 225 | 364 |
| 849 | 46 | 465 |
| 850 | 1150 | 224 |
| 851 | 148 | 310 |
| 852 | 163 | 249 |
| 853 | 92 | 322 |
| 854 | 136 | 326 |
| 855 | 80 | 341 |
| 856 | 35 | 357 |
| 857 | 407 | 337 |
| 858 | 95 | 308 |
| 859 | 36 | 342 |
| 860 | 578 | 288 |
| 861 | 722 | 384 |
| 862 | 921 | 335 |
| 863 | 587 | 326 |
| 864 | 617 | 385 |
| 865 | 103 | 356 |
| 866 | 337 | 324 |
| 867 | 31 | 413 |
| 868 | 18 | 397 |
| 869 | 358 | 305 |
| 870 | 201 | 316 |
| 871 | 32 | 378 |
| 872 | 535 | 318 |
| 873 | 41 | 336 |
| 874 | 66 | 332 |
| 875 | 21 | 333 |
| 876 | 113 | 317 |
| 877 | 186 | 361 |
| 878 | 27 | 336 |
| 879 | 63 | 360 |
| 880 | 59 | 359 |
| 881 | 309 | 337 |
| 882 | 473 | 367 |
| 883 | 111 | 344 |
| 884 | 96 | 385 |
| 885 | 78 | 328 |
| 886 | 15 | 359 |
| 887 | 111 | 340 |
| 888 | 170 | 410 |
| 889 | 34 | 403 |
| 890 | 34 | 409 |
| 891 | 1490 | 402 |
| 892 | 467 | 442 |
| 893 | 216 | 394 |
| 894 | 339 | 431 |
| 895 | 136 | 409 |
| 896 | 188 | 427 |
| 897 | 32 | 454 |
| 898 | 22 | 440 |
| 899 | 819 | 294 |
| 900 | 831 | 374 |
| 901 | 736 | 390 |
| 902 | 55 | 453 |
| 903 | 51 | 414 |
| 904 | 722 | 365 |
| 905 | 311 | 415 |
| 906 | 294 | 433 |
| 907 | 425 | 454 |
| 908 | 94 | 401 |
| 909 | 17 | 430 |
| 910 | 477 | 423 |
| 911 | 531 | 341 |
| 912 | 272 | 332 |
| 913 | 294 | 390 |
| 914 | 368 | 358 |
| 915 | 334 | 361 |
| 916 | 196 | 470 |
| 917 | 487 | 398 |
| 918 | 42 | 462 |
| 919 | 104 | 443 |
| 920 | 175 | 380 |
| 921 | 231 | 393 |
| 922 | 302 | 396 |
| 923 | 26 | 500 |
| 924 | 33 | 409 |
| 925 | 92 | 286 |
| 926 | 759 | 416 |
| 927 | 27 | 492 |
| 928 | 971 | 247 |
| 929 | 306 | 513 |
| 930 | 142 | 460 |
| 931 | 298 | 507 |
| 932 | 373 | 491 |
| 933 | 399 | 485 |
| 934 | 37 | 528 |
| 935 | 24 | 496 |
| 936 | 26 | 469 |
| 937 | 59 | 460 |
| 938 | 245 | 498 |
| 939 | 737 | 288 |
| 940 | 673 | 298 |
| 941 | 135 | 459 |
| 942 | 194 | 460 |
| 943 | 240 | 471 |
| 944 | 77 | 336 |
| 945 | 33 | 502 |
| 946 | 447 | 404 |
| 947 | 281 | 458 |
| 948 | 61 | 501 |
| 949 | 113 | 518 |
| 950 | 201 | 464 |
| 951 | 165 | 405 |
| 952 | 339 | 438 |
| 953 | 268 | 394 |
| 954 | 382 | 383 |
| 955 | 381 | 394 |
| 956 | 508 | 412 |
| 957 | 192 | 451 |
| 958 | 338 | 439 |
| 959 | 222 | 413 |
| 960 | 85 | 507 |
| 961 | 353 | 453 |
| 962 | 103 | 453 |
| 963 | 267 | 447 |
| 964 | 21 | 418 |
| 965 | 32 | 453 |
| 966 | 82 | 404 |
| 967 | 92 | 472 |
| 968 | 226 | 478 |
| 969 | 142 | 452 |
| 970 | 109 | 463 |
| 971 | 163 | 482 |
| 972 | 128 | 436 |
| 973 | 117 | 446 |
| 974 | 703 | 440 |
| 975 | 119 | 497 |
| 976 | 179 | 348 |
| 977 | 281 | 410 |
| 978 | 129 | 457 |
| 979 | 209 | 394 |
| 980 | 53 | 435 |
| 981 | 133 | 448 |
| 982 | 236 | 530 |
| 983 | 270 | 506 |

Reference Compound 1: Apicidin was purchased from Sigma-Aldrich and evaluated in the above DiscoveRx assay under the same conditions; the compound showed an EC₅₀: >20000 nmol/l.

1. A compound of Formula (I) 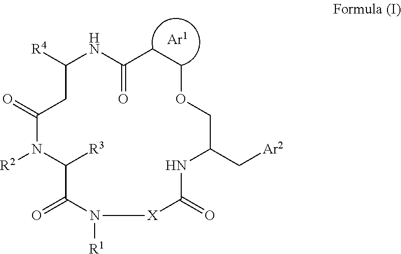
wherein
X represents —CR^{X1}R^{X2}, wherein
R^{X1} and R^{X2} together with the carbon atom to which they are attached form a ring which is:
C₃₋₆-cycloalkan-1,1-diyl;
C₅₋₆-cycloalkan-1,1-diyl which is fused to a benzene ring;
C₃₋₆-cycloalkan-1,1-diyl, wherein said C₃₋₆-cycloalkan-1,1-diyl group independently is mono-substituted with C₁₋₃-alkoxy, fluoro, or hydroxy; or di-substituted with fluoro;
C₄₋₆-heterocycloalkan-diyl, wherein said C₄₋₆-heterocycloalkan-diyl contains one ring nitrogen atom, wherein said nitrogen when having a free valency is unsubstituted or mono-substituted wherein the substitutents are independently selected from C₁₋₄-alkyl, and —COO—C₁₋₃-alkyl; or
C₄₋₆-heterocycloalkan-diyl, wherein said C₄₋₆-heterocycloalkan-diyl contains one ring oxygen atom;
R^{X1} and R^{X2} both independently represent C₁₋₄-alkyl; or
R^{X1} represents hydrogen, and
R^{X2} represents
hydrogen;
C₁₋₆-alkyl;
C₁₋₄-fluoroalkyl;
C₃₋₆-cycloalkyl;
C₁₋₃-alkyl wherein said C₁₋₃-alkyl is mono-substituted with
hydroxy;
C₁₋₄-alkoxy;
-L^{X1}-C₃₋₆cycloalkyl wherein said C₃₋₆-cycloalkyl is unsubstituted or di-substituted with fluoro; and wherein L^{X1} independently represents a direct bond or oxygen;
C₄₋₆-heterocycloalkyl wherein said C₄₋₆-heterocycloalkyl contains one ring oxygen atom;
-NR^{N1}R^{N2} wherein R^{N1} and R^{N2} together with the nitrogen form a 4- to 6-membered carbocyclic ring comprising the nitrogen atom, wherein said ring is mono- or di-substituted with fluoro;
a partially aromatic bicyclic ring, which is 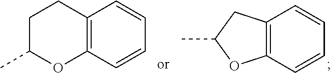
or
-L^{X2}-Ar^{X2} wherein—
L^{X2} independently represents a direct bond, C₁₋₃-alkylene, —C₁₋₃-alkylene-O—*, or —C₁₋₃-alkylene-O—C₁₋₂-alkylene-*; wherein the asterisks indicate the bond that is attached to the group Ar^{X2}; and
ArX² independently represents aryl, or 5- to 10-membered heteroaryl; wherein said group Ar^{X2} independently is unsubstituted, or mono-, or di-substituted wherein the substituents are independently selected from
C₁₋₄-alkyl;
C₁₋₃-alkoxy;
halogen;
C₃₋₆-cycloalkyl;
C₁₋₃-fluoroalkyl; and
Ar^{X3} wherein Ar^{X3} independently represents phenyl, or 5- or 6-membered heteroaryl; wherein said group Ar^{X3} independently is unsubstituted, or mono-, or di-substituted wherein the substituents independently are selected from C₁₋₃-alkyl, C₁₋₃-alkoxy, C₁₋₃-alkoxy-C₂₋₃-alkyl, C₃₋₅-cycloalkyl, C₁₋₃-fluoroalkyl, and halogen;
and
R¹ independently represents
hydrogen;
—C₁₋₆-alkyl;
—C₂₋₆-alkyl wherein said C₂₋₆-alkyl is mono-substituted with hydroxy, or C₁₋₄-alkoxy;
—C₁₋₆-alkyl wherein said C₁₋₆-alkyl is mono-substituted with R¹¹; wherein R¹¹ independently represents
a saturated 5- or 6-membered heterocycloalkyl containing one or two ring heteroatoms wherein said heteroatoms are independently selected from nitrogen and oxygen, wherein said 5- or 6-membered heterocycloalkyl is independently unsubstituted, mono- or di-substituted, wherein the substituents independently are selected from C₁₋₄-alkyl, halogen, and benzyl;
C₃₋₆-cycloalkyl, wherein said C₃₋₆-cycloalkyl is unsubstituted or mono-substituted with C₁₋₄-alkoxy;
phenyl or 5- or 6-membered heteroaryl, wherein said phenyl or 5- or 6-membered heteroaryl independently is unsubstituted, mono- or di-substituted wherein the substitutents independently are selected from C₁₋₄-alkyl, C₁₋₄-alkoxy, C₁₋₃-fluoroalkyl, C₁₋₃-fluoroalkoxy, halogen, cyano, and morpholin-4-yl;
benzyloxy;
a spirocyclic fragment, which is 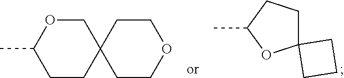
a saturated bicyclic ring, which is 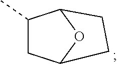
or
a partially aromatic bicyclic ring, which is 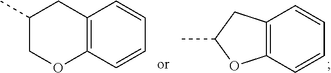
or the fragment 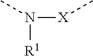
represents a heterocyclic ring which is 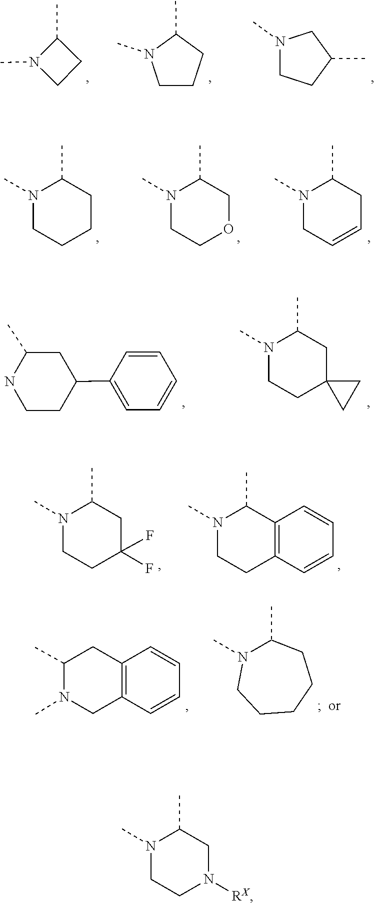
wherein R^{X} represents
hydrogen;
C₁₋₄-alkyl;
C₃₋₆-cycloalkyl;
C₁₋₄-alkyl, wherein said C₁₋₄-alkyl is mono-substituted with C₃₋₄-cycloalkyl;
C₂₋₄-alkyl, wherein said C₂₋₄-alkyl is mono-substituted with hydroxy or C₁₋₃-alkoxy;
phenyl or 5- or 6-membered heteroaryl, wherein said phenyl or 5- or 6-membered heteroaryl independently is unsubstituted, mono- or di-substituted wherein the substituents independently are selected from C₁₋₄-alkyl, C₁₋₃-alkoxy, C₁₋₃-fluoroalkyl, C₁₋₃-fluoroalkoxy, cyano or halogen; 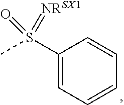
wherein R^{SX1} represents hydrogen or —CO—O—C₁₋₄-alkyl;
—CO—R^{OX1}, or —SO₂—R^{OX1}; wherein R^{OX1} independently represents
C₁₋₄-alkyl;
C₁₋₃-alkyl wherein said C₁₋₃-alkyl is mono-substituted with C₁₋₃-alkoxy, tetrahydropyranyl, morpholin-4-yl, phenyl, 10-membered heteroaryl, or —NR^{ONX1}R^{ONX2} wherein R^{ONX1} and R^{ONX2} independently represent hydrogen or C₁₋₃-alkyl;
tetrahydropyranyl;
phenyl or 5- or 6-membered heteroaryl, wherein said phenyl or 5- or 6-membered heteroaryl independently is unsubstituted, mono- or di-substituted wherein the substituents independently are selected from C₁₋₄-alkyl, C₁₋₃-alkoxy, C₁₋₃-fluoroalkyl, C₁₋₃-fluoroalkoxy, cyano or halogen; or
a group of the structure (R^{X-A}): 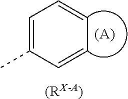
wherein (A) represents a non-aromatic 5- or 6-membered ring fused to the phenyl group, wherein ring (A) comprises two heteroatoms independently selected from oxygen and nitrogen; wherein said ring (A) independently is unsubstituted or mono-substituted wherein the substitutents independently are selected from oxo and C₁₋₃-alkyl; or
—CO—O—R^{OX2}; wherein R^{OX2} represents
C₁₋₄-alkyl;
2,2,2-trichloroethyl; or
tetrahydropyranyl;
R² represents C₁₋₄-alkyl;
R³ represents hydrogen; C₁₋₆-alkyl; —CH₂—C₃₋₆-cycloalkyl; or C₂₋₄-alkynyl;
R⁴ represents a group —CO—NH—R⁴¹; wherein R⁴¹ represents
C₂₋₆-alkyl, which is mono-substituted with C₁₋₄-alkoxy, C₁₋₄-fluoroalkoxy, or hydroxy;
C₁₋₃-alkoxy-C₂₋₃-alkylene-O—CH₂—CH₂—;
CH₂—CH₂—C₅₋₆-heterocycloalkyl, wherein said C₅₋₆-heterocycloalkyl contains one ring oxygen atom, wherein said C₅₋₆-heterocyclyl is unsubstituted, mono- or di-substituted with C₁₋₄-alkyl;
-L¹-aryl; wherein L¹ represents —CH₂—CH₂—, —CH₂—CH₂—O—*, —CH₂—CF₂—*, —CH₂-(cyclopropan-1,1-diyl)-*, —CH(CH₂—OH)—CH₂—*, or —CH₂—CH(OH)—*; wherein asterisks indicate the bond with which L¹ is attached to the aryl; wherein aryl represents phenyl or naphthyl; wherein said aryl is unsubstituted, mono-, di- or tri-substituted, wherein the substituents are independently C₁₋₄-alkyl, C₁₋₄-alkoxy, C₁₋₃-fluoroalkyl, C₁₋₃-fluoroalkoxy, halogen, cyano, C₃₋₆-cycloalkyl, C₃₋₆-cycloalkyl-methyl, C₁₋₃-alkoxy-C₁₋₃-alkyl, hydroxy-C₁₋₃-alkyl, C₂₋₃-alkynyl, morpholin-4-yl, C₁₋₃-alkyl-SO₂—, 5- or 6-membered heteroaryl, or —NR^{N41}R^{N42}, wherein independently R^{N41} is hydrogen or C₁₋₄-alkyl, and R^{N42} is hydrogen or C₁₋₄-alkyl;
-L²-HET; wherein L² represents —CH₂—CH₂—, —CH₂—CH₂—O—*, —CH₂—CF₂—*, —CH₂-(cyclopropan-1,1-diyl)-*, —CH(CH₂—OH)—CH₂—*, or —CH₂—CH(OH)—*; wherein asterisks indicate the bond with which L² is attached to HET; wherein HET represents 5- to 10-membered heteroaryl, wherein said HET is independently unsubstituted, mono-, di- or tri-substituted, wherein the substituents are independently C₁₋₄-alkyl; C₁₋₄-alkoxy; C₁₋₃-fluoroalkyl; C₁₋₃-fluoroalkoxy; halogen; cyano; C₃₋₆-cycloalkyl; C₃₋₆-cycloalkyl-methyl; C₁₋₃-alkoxy-C₁₋₃-alkyl; hydroxy-C₁₋₃-alkyl; C₂₋₃-alkynyl; benzyl; or phenyl which is unsubstituted, mono- or di-substituted wherein the substituents independently are C₁₋₄-alkyl, C₁₋₄-alkoxy, or C₁₋₄-fluoroalkoxy;
—CH₂—CH₂—HCy¹, wherein HCy¹ represents a partially aromatic bicyclic ring system consisting of a phenyl ring which is fused to a 5- to 7-membered saturated heterocyclic ring containing one or two heteroatoms independently selected from oxygen and nitrogen, wherein, if present, said nitrogen when having a free valency is unsubstituted or mono-substituted with C₁₋₄-alkyl; and wherein the phenyl ring of said partially aromatic bicyclic ring system is unsubstituted, mono-, di- or tri-substituted, wherein the substituents are independently C₁₋₄-alkyl, C₁₋₄-alkoxy, C₁₋₃-fluoroalkyl, C₁₋₃-fluoroalkoxy, halogen, or cyano;
—CH₂—CH₂—HCy², wherein HCy² represents a partially aromatic bicyclic ring system consisting of a 5-membered heteroaryl which is fused to a 5- to 7-membered saturated carbocyclic ring; or
-L³-HCy³, wherein L³ represents a direct bond, or —CH₂—; wherein HCy³ represents a partially aromatic bicyclic ring system consisting of a phenyl ring which is fused to a 5- to 7-membered saturated heterocyclic ring containing one oxygen atom; wherein L³ is attached to said group HCy³ at a carbon atom which is part of said 5- to 7-membered saturated heterocyclic ring; and wherein the phenyl ring of said partially aromatic bicyclic ring system is unsubstituted, or mono-substituted with C₁₋₄-alkyl, C₁₋₄-alkoxy, C₁₋₃-fluoroalkyl, C₁₋₃-fluoroalkoxy, halogen, or cyano;
Ar¹ represents
5- or 6-membered heteroarylene wherein said 5- or 6-membered heteroarylene is unsubstituted;
phenylene, or 5- or 6-membered heteroarylene; wherein said phenylene, or 5- or 6-membered heteroarylene independently is mono-, di- or tri-substituted, wherein the substituents are independently selected from C₁₋₄-alkyl, C₁₋₄-alkoxy, C₁₋₃-fluoroalkyl, C₁₋₃-fluoroalkoxy, cyano, and halogen;
phenylene wherein said phenylene is fused to a 5- or 6-membered saturated heterocyclic ring containing one or two oxygen atoms, wherein said 5- or 6-membered saturated heterocyclic ring independently is unsubstituted or di-substituted with fluoro; or
a bicyclic aromatic ring selected from naphthylene and 8- to 10-membered bicyclic heteroarylene; wherein said bicyclic aromatic ring independently is unsubstituted, mono-, or di-substituted, wherein the substituents are independently selected from C₁₋₄-alkyl, C₁₋₃-fluoroalkyl, C₁₋₄-alkoxy, C₁₋₃-fluoroalkoxy, cyano, and halogen; or
quinoline-diyl, wherein said quinoline-diyl is present in form of the respective N-oxide; wherein said quinoline-diyl N-oxide is unsubstituted, or said quinoline-diyl N-oxide is mono-substituted with methyl or fluoro;
and
Ar² represents
phenyl or naphthyl, wherein said phenyl or naphthyl independently is unsubstituted, mono- or di-substituted wherein the substituents are independently selected from C₁₋₄-alkyl, C₁₋₃-fluoroalkyl, halogen, cyano, C₁₋₆-alkoxy, and C₁₋₃-fluoroalkoxy;
5- or 6-membered heteroaryl, wherein said 5- or 6-membered heteroaryl independently is unsubstituted or mono-substituted wherein the substituents are independently selected from C₁₋₄-alkyl, C₁₋₃-fluoroalkyl, halogen, cyano, C₁₋₆-alkoxy, and C₁₋₃-fluoroalkoxy; or
9- or 10-membered heteroaryl;
or a pharmaceutically acceptable salt thereof.

2. A compound of Formula (I) as defined for claim 1, wherein the compounds are also compounds of Formula (I_{E}): 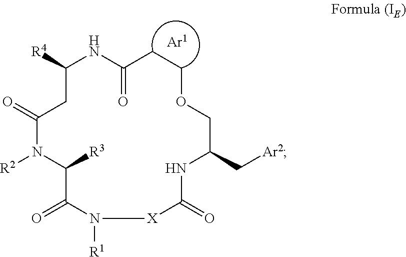
or a pharmaceutically acceptable salt thereof.

3. A compound according to claim 1; wherein
X represents —CR^{X1}R^{X2}, wherein
R^{X1} and R^{X2} together with the carbon atom to which they are attached form a ring which is:
C₃₋₆-cycloalkan-1,1-diyl-;
C₅₋₆-cycloalkan-1,1-diyl- which is fused to a benzene ring; or
C₃₋₆-cycloalkan-1,1-diyl-, wherein said C₃₋₆-cycloalkan-1,1-diyl group is mono-substituted with C₁₋₃-alkoxy, or di-substituted with fluoro;
R^{X1} and R^{X2} both independently represent C₁₋₄-alkyl; or
R^{X1} represents hydrogen, and
R^{X2} represents
hydrogen;
C₁₋₆-alkyl;
C₁₋₄-fluoroalkyl;
C₃₋₆-cycloalkyl;
C₁₋₃-alkyl wherein said C₁₋₃-alkyl is mono-substituted with
hydroxy;
C₁₋₄-alkoxy;
-L^{X1}-C₃₋₆cycloalkyl wherein said C₃₋₆-cycloalkyl is unsubstituted or di-substituted with fluoro; and wherein L^{X1} independently represents a direct bond or oxygen;
C₄₋₆-heterocycloalkyl wherein said C₄₋₆-heterocycloalkyl contains one ring oxygen atom;
—NR^{N1}R^{N2} wherein R^{N1} and R^{N2} together with the nitrogen form a 4- to 6-membered carbocyclic ring comprising the nitrogen atom, wherein said ring is mono- or di-substituted with fluoro;
-L^{X2}-Ar^{X2}wherein—
L^{X2} independently represents a direct bond, C₁₋₃-alkylene, —C₁₋₃-alkylene-O—*, or —C₁₋₃-alkylene-O—C₁₋₂-alkylene-*; wherein the asterisks indicate the bond that is attached to the group Ar^{X2}; and
ArX² independently represents aryl, or 5- to 10-membered heteroaryl; wherein said group Ar^{X2} independently is unsubstituted, or mono-, or di-substituted wherein the substituents are independently selected from
C₁₋₄-alkyl;
C₁₋₃-alkoxy;
halogen;
cyano;
C₃₋₆-cycloalkyl;
C₁₋₃-fluoroalkyl; and
Ar^{X3} wherein Ar^{X3} independently represents phenyl, or 5- or 6-membered heteroaryl; wherein said group Ar^{X3} independently is unsubstituted, or mono-, or di-substituted wherein the substituents independently are selected from C₁₋₃-alkyl, C₁₋₃-alkoxy, C₁₋₃-alkoxy-C₂₋₃-alkyl, C₃₋₅-cycloalkyl, C₁₋₃-fluoroalkyl, and halogen;
and
R¹ independently represents hydrogen or —C₁₋₃-alkyl; or
X represents —CR^{X1}R^{X2}, wherein
R^{X1} represents hydrogen, and
R^{X2} represents hydrogen, or methyl; or
R^{X1} and R^{X2} together with the carbon atom to which they are attached form a ring which is C₃₋₅-cycloalkan-1,1-diyl-;
and
R¹ independently represents
—C₄₋₆-alkyl;
C₂₋₆-alkyl, wherein said C₂₋₆-alkyl is mono-substituted with C₁₋₄-alkoxy;
C₃₋₆-alkyl, wherein said C₃₋₆-alkyl is mono-substituted with phenyl, or benzyloxy;
(CH₂)ₘ—R¹¹ wherein m represents the integer 1 or 2; and R¹¹ independently represents
a saturated 5- or 6-membered heterocycloalkyl containing one or two ring oxygen atoms, wherein said 5- or 6-membered heterocycloalkyl is independently unsubstituted, mono- or di-substituted with C₁₋₄-alkyl;
C₃₋₆-cycloalkyl, wherein said C₃₋₆-cycloalkyl is unsubstituted or mono-substituted with C₁₋₄-alkoxy;
phenyl or 5- or 6-membered heteroaryl, wherein said phenyl or 5- or 6-membered heteroaryl independently is unsubstituted, mono- or di-substituted wherein the substituents independently are selected from C₁₋₄-alkyl, C₁₋₄-alkoxy, C₁₋₃-fluoroalkyl, C₁₋₃-fluoroalkoxy, cyano or halogen;
a spirocyclic fragment, which is 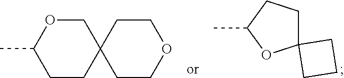
a saturated bicyclic ring, which is 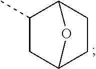
or
a partially aromatic bicyclic ring, which is 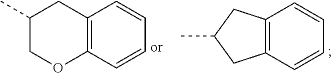
or the fragment 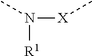
represents a heterocyclic ring which is 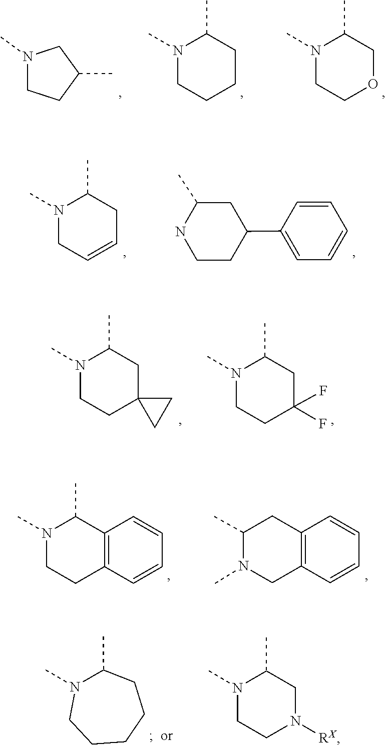
wherein R^{X} represents
hydrogen;
C₁₋₄-alkyl;
C₃₋₄-cycloalkyl;
C₁₋₄-alkyl, wherein said C₁₋₄-alkyl is mono-substituted with C₃₋₄-cycloalkyl;
C₂₋₄-alkyl, wherein said C₂₋₄-alkyl is mono-substituted with hydroxy, or C₁₋₃-alkoxy;
phenyl or 5- or 6-membered heteroaryl, wherein said phenyl or 5- or 6-membered heteroaryl independently is unsubstituted, mono- or di-substituted wherein the substituents independently are selected from C₁₋₄-alkyl, C₁₋₃-alkoxy, C₁₋₃-fluoroalkyl, C₁₋₃-fluoroalkoxy, cyano or halogen; 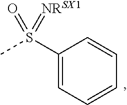
wherein R^{SX1} represents hydrogen or —CO—O—C₁₋₄-alkyl;
CO—R^{OX1}, or —SO₂—R^{OX1}; wherein R^{OX1} independently represents
C₁₋₄-alkyl;
C₁₋₃-alkyl wherein said C₁₋₃-alkyl is mono-substituted with C₁₋₃-alkoxy, tetrahydropyranyl, morpholin-4-yl, phenyl, 10-membered heteroaryl, or —NR^{ONX1}R^{ONX2} wherein R^{ONX1} and R^{ONX2} independently represent hydrogen or C₁₋₃-alkyl;
tetrahydropyranyl;
phenyl or 5- or 6-membered heteroaryl, wherein said phenyl or 5- or 6-membered heteroaryl independently is unsubstituted, mono- or di-substituted wherein the substituents independently are selected from C₁₋₄-alkyl, C₁₋₃-alkoxy, C₁₋₃-fluoroalkyl, C₁₋₃-fluoroalkoxy, cyano or halogen; or
a group of the structure (R^{X-A}): 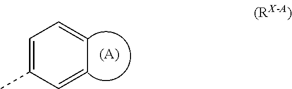
wherein (A) represents a non-aromatic 5- or 6-membered ring fused to the phenyl group, wherein ring (A) comprises two heteroatoms independently selected from oxygen and nitrogen; wherein said ring (A) independently is unsubstituted or mono-substituted wherein the substitutents independently are selected from oxo and C₁₋₃-alkyl; or
—CO—O—R^{OX2}; wherein R^{OX2} represents
C₁₋₄-alkyl;
2,2,2-trichloroethyl; or
tetrahydropyranyl;
or a pharmaceutically acceptable salt thereof.

4. A compound according to claim 1; wherein the fragment 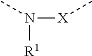
represents a group selected from:
A) 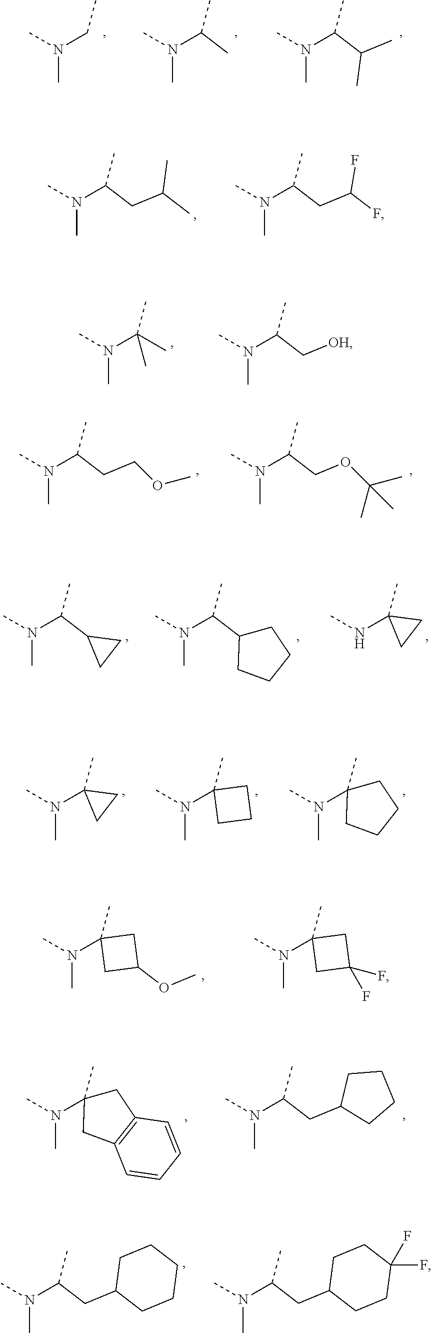 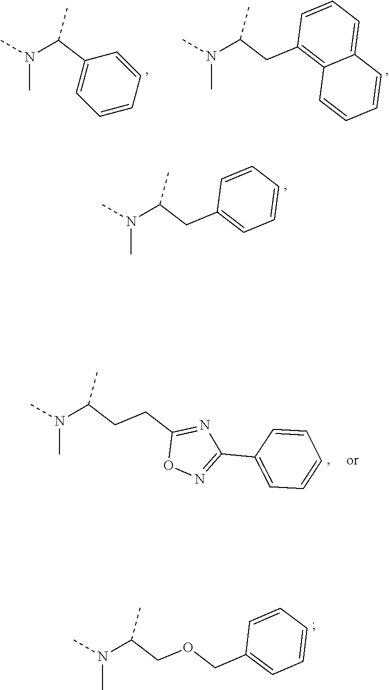
B) 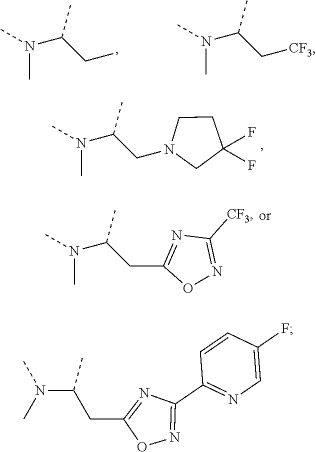
C) 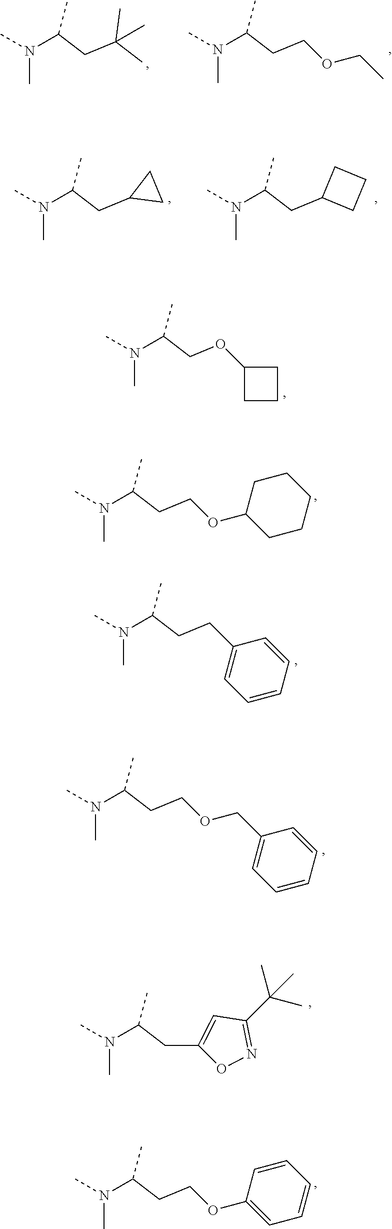 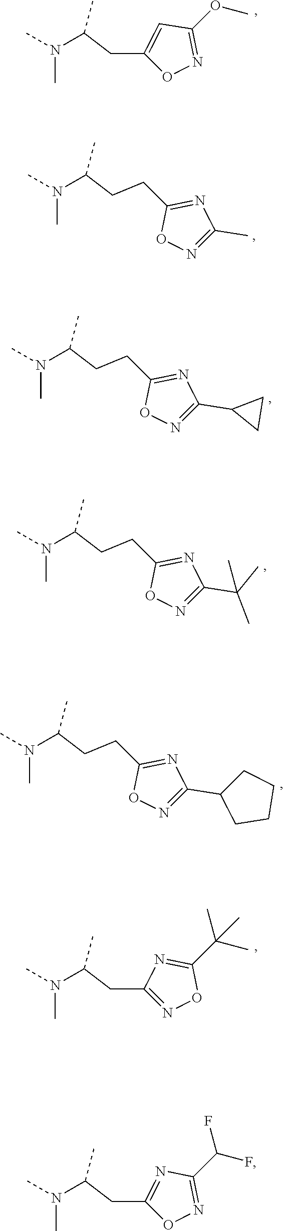 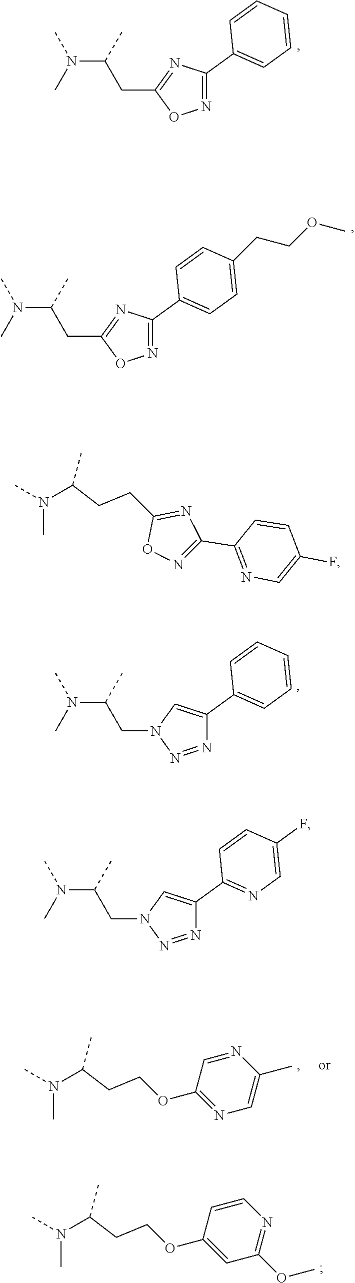
D) 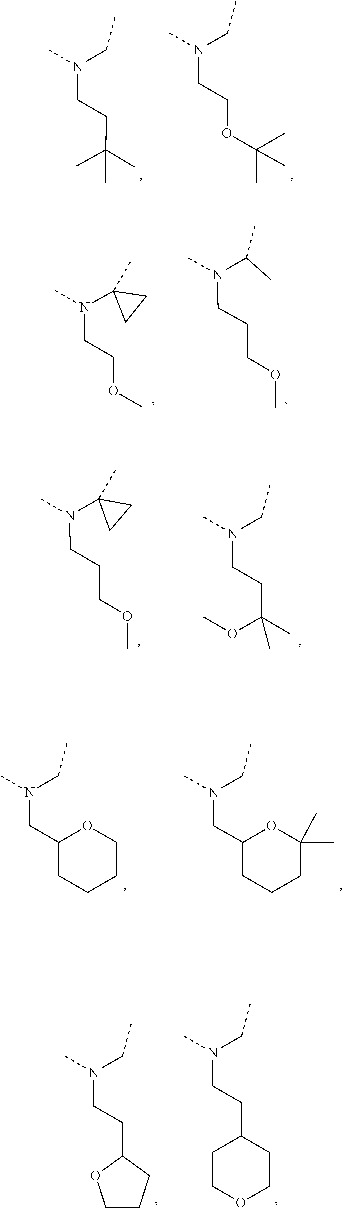 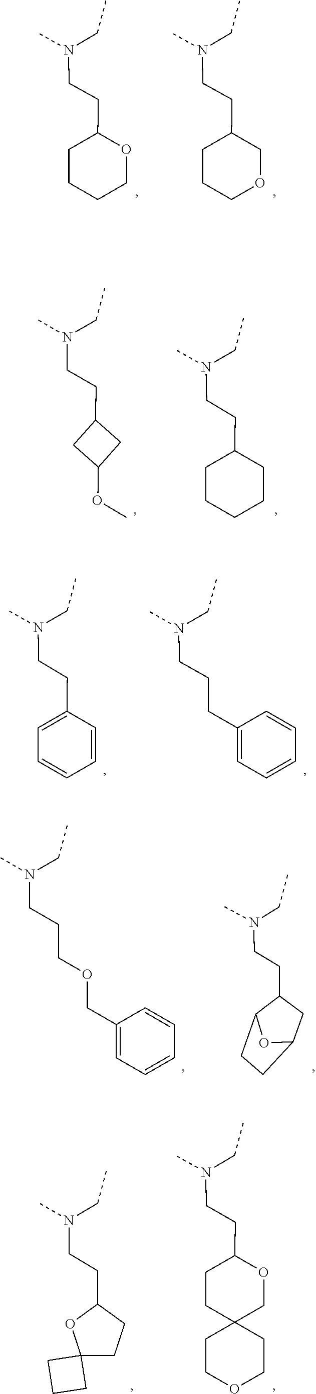 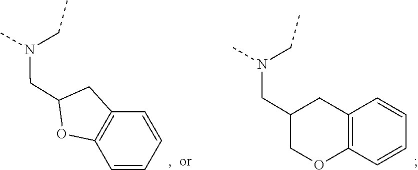
E) 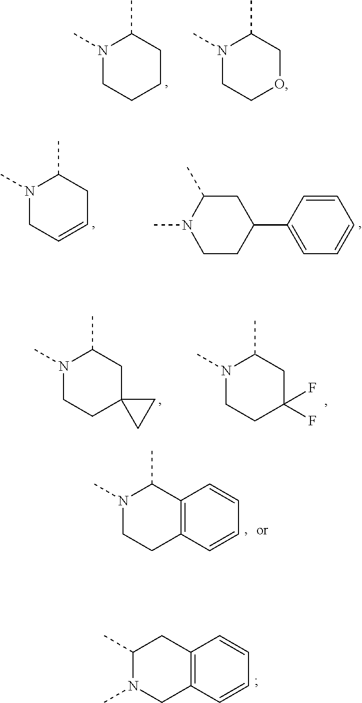
F) 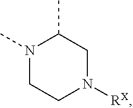
wherein R^{X} represents
C₁₋₄-alkyl;
C₃₋₄-cycloalkyl;
C₁₋₄-alkyl, wherein said C₁₋₄-alkyl is mono-substituted with C₃₋₄-cycloalkyl;
C₁₋₄-alkyl, wherein said C₁₋₄-alkyl is mono-substituted with hydroxy, or C₁₋₃-alkoxy;
phenyl;
6-membered heteroaryl, wherein said 6-membered heteroaryl is unsubstituted or mono-substituted with halogen; 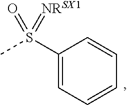
wherein R^{SX1} represents —CO—O—C₁₋₄-alkyl;
—CO—R^{OX1}, or —SO₂—R^{OX1}; wherein R^{OX1} independently represents
C₁₋₄-alkyl;
C₁₋₃-alkyl wherein said C₁₋₃-alkyl is mono-substituted with C₁₋₃-alkoxy, tetrahydropyranyl, morpholin-4-yl, phenyl, 10-membered heteroaryl, or —NR^{ONX1}R^{ONX2} wherein R^{ONX1} and R^{ONX2} independently represent hydrogen or C₁₋₃-alkyl;
tetrahydropyranyl;
phenyl which is unsubstituted, mono- or di-substituted wherein the substituents independently are C₁₋₃-alkoxy, C₁₋₃-fluoroalkoxy or halogen;
5- or 6-membered heteroaryl wherein said 5- or 6-membered heteroaryl is independently unsubstituted or mono-substituted with C₁₋₃-alkoxy; or
a group of the structure (R^{X-A}): 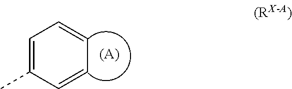
wherein (A) represents a non-aromatic 5- or 6-membered ring fused to the phenyl group, wherein ring (A) comprises two heteroatoms independently selected from oxygen and nitrogen; wherein said ring (A) independently is unsubstituted or mono-substituted wherein the substitutents independently are selected from oxo and C₁₋₃-alkyl; or
—CO—O—R^{OX2}; wherein R^{OX2} represents
C₁₋₄-alkyl;
2,2,2-trichloroethyl; or
tetrahydropyranyl;
G) 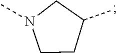
or H) 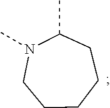
or a pharmaceutically acceptable salt thereof.

5. A compound according to claim 1; wherein R² represents methyl;
or a pharmaceutically acceptable salt thereof.

6. A compound according to claim 1; wherein R³ represents isobutyl;
or a pharmaceutically acceptable salt thereof.

7. A compound according to claim 1; wherein R⁴ represents a group —CO—NH—R⁴¹; wherein R⁴¹ represents
C₂₋₆-alkyl, which is mono-substituted with C₁₋₄-alkoxy, or C₁₋₄-fluoroalkoxy;
C₁₋₃-alkoxy-C₂₋₃-alkylene-O—CH₂—CH₂—;
—CH₂—CH₂—C₅₋₆-heterocycloalkyl, wherein said C₅₋₆-heterocycloalkyl contains one ring oxygen atom, wherein said C₅₋₆-heterocyclyl is unsubstituted, mono- or di-substituted with C₁₋₄-alkyl;
-L¹-aryl; wherein L¹ represents —CH₂—CH₂—, —CH₂—CH₂—O—* or —CH₂—CF₂—*, —CH₂-(cyclopropan-1,1-diyl)-*, —CH(CH₂—OH)—CH₂—*, or —CH₂—CH(OH)—*; wherein asterisks indicate the bond with which L¹ is attached to the aryl; wherein aryl represents phenyl; wherein said aryl independently is unsubstituted, mono-, di- or tri-substituted, wherein the substituents are independently C₁₋₄-alkyl, C₁₋₄-alkoxy, C₁₋₃-fluoroalkyl, C₁₋₃-fluoroalkoxy, halogen, cyano, hydroxy-C₁₋₃-alkyl, C₂₋₃-alkynyl, morpholin-4-yl, C₁₋₃-alkyl-SO₂—, 5- or 6-membered heteroaryl, or —NR^{N41}R^{N42}, wherein independently R^{N4}1 is hydrogen or C₁₋₄-alkyl, and R^{N42} is hydrogen or C₁₋₄-alkyl;
-L²-HET¹; wherein L² represents —CH₂—CH₂—, —CH₂—CF₂—*, —CH₂-(cyclopropan-1,1-diyl)-*, or —CH₂—CH(OH)—*; wherein asterisks indicate the bond with which L² is attached to HET¹; wherein HET¹ represents 5- or 6-membered heteroaryl, wherein said 5- or 6-membered heteroaryl is independently unsubstituted, mono-, or di-substituted, wherein the substituents are independently C₁₋₄-alkyl; C₁₋₄-alkoxy; C₁₋₃-fluoroalkyl; halogen; C₃₋₆-cycloalkyl; C₃₋₆-cycloalkyl-methyl; C₁₋₃-alkoxy-C₁₋₃-alkyl; C₂₋₃-alkynyl, benzyl; or phenyl which is unsubstituted, mono- or di-substituted wherein the substituents independently are C₁₋₄-alkyl, C₁₋₄-alkoxy, or C₁₋₄-fluoroalkoxy;
—CH₂—CH₂—HET², wherein HET² represents 9- or 10-membered bicyclic heteroaryl, wherein said HET² is unsubstituted;
—CH₂—CH₂—HCy¹, wherein HCy¹ represents a partially aromatic bicyclic ring system consisting of a phenyl ring which is fused to a 5- to 7-membered saturated heterocyclic ring containing one or two heteroatoms independently selected from oxygen and nitrogen, wherein, if present, said nitrogen when having a free valency is unsubstituted or mono-substituted with C₁₋₄-alkyl; and wherein the phenyl ring of said partially aromatic bicyclic ring system is unsubstituted, mono-, or di-substituted, wherein the substituents are independently C₁₋₄-alkyl, C₁₋₄-alkoxy, or halogen; or
—CH₂—CH₂—HCy², wherein HCy² represents a partially aromatic bicyclic ring system consisting of a 5-membered heteroaryl which is fused to a 5- to 7-membered saturated carbocyclic ring;
or a pharmaceutically acceptable salt thereof.

8. A compound according to claim 1; wherein Ar² represents phenyl;
or a pharmaceutically acceptable salt thereof.

9. A compound according to claim 1, wherein Ar¹ represents
phenylene, or 5- or 6-membered heteroarylene; wherein said phenylene, or 5- or 6-membered heteroarylene independently is mono-, or di-substituted, wherein the substituents are independently selected from C₁₋₄-alkyl, C₁₋₄-alkoxy, and halogen;
phenylene wherein said phenylene is fused to a 5- or 6-membered saturated heterocyclic ring containing one or two oxygen atoms, wherein said 5- or 6-membered saturated heterocyclic ring independently is unsubstituted; or
a bicyclic aromatic ring selected from naphthylene and 8- to 10-membered bicyclic heteroarylene; wherein said bicyclic aromatic ring independently is unsubstituted, or mono-substituted, wherein the substituents are independently selected from C₁₋₄-alkyl, and halogen;
or a pharmaceutically acceptable salt thereof.

10. A compound according to claim 1 wherein said compound is the compound of example 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; 105; 106; 107; 108; 109; 110; 111; 112; 113; 114; 115; 116; 117; 118; 119; 120; 121; 122; 123; 124; 125; 126; 127; 128; 129; 130; 131; 132; 133; 134; 135; 136; 137; 138; 139; 140; 141; 142; 143; 144; 145; 146; 147; 148; 149; 150; 151; 152; 153; 154; 155; 156; 157; 158; 159; 160; 161; 162; 163; 164; 165; 166; 167; 168; 169; 170; 171; 172; 173; 174; 175; 176; 177; 178; 179; 180; 181; 182; 183; 184; 185; 186; 187; 188; 189; 190; 191; 192; 193; 194; 195; 196; 197; 198; 199; 200; 201; 202; 203; 204; 205; 206; 207; 208; 209; 210; 211; 212; 213; 214; 215; 216; 217; 218; 219; 220; 221; 222; 223; 224; 225; 226; 227; 228; 229; 230; 231; 232; 233; 234; 235; 236; 237; 238; 239; 240; 241; 242; 243; 244; 245; 246; 247; 248; 249; 250; 251; 252; 253; 254; 255; 256; 257; 258; 259; 260; 261; 262; 263; 264; 265; 266; 267; 268; 269; 270; 271; 272; 273; 274; 275; 276; 277; 278; 279; 280; 281; 282; 283; 284; 285; 286; 287; 288; 289; 290; 291; 292; 293; 294; 295; 296; 297; 298; 299; 300; 301; 302; 303; 304; 305; 306; 307; 308; 309; 310; 311; 312; 313; 314; 315; 316; 317; 318; 319; 320; 321; 322; 323; 324; 325; 326; 327; 328; 329; 330; 331; 332; 333; 334; 335; 336; 337; 338; 339; 340; 341; 342; 343; 344; 345; 346; 347; 348; 349; 350; 351; 352; 353; 354; 355; 356; 357; 358; 359; 360; 361; 362; 363; 364; 365; 366; 367; 368; 369; 370; 371; 372; 373; 374; 375; 376; 377; 378; 379; 380; 381; 382; 383; 384; 385; 386; 387; 388; 389; 390; 391; 392; 393; 394; 395; 396; 397; 398; 399; 400; 401; 402; 403; 404; 405; 406; 407; 408; 409; 410; 411; 412; 413; 414; 415; 416; 417; 418; 419; 420; 421; 422; 423; 424; 425; 426; 427; 428; 429; 430; 431; 432; 433; 434; 435; 436; 437; 438; 439; 440; 441; 442; 443; 444; 445; 446; 447; 448; 449; 450; 451; 452; 453; 454; 455; 456; 457; 458; 459; 460; 461; 462; 463; 464; 465; 466; 467; 468; 469; 470; 471; 472; 473; 474; 475; 476; 477; 478; 479; 480; 481; 482; 483; 484; 485; 486; 487; 488; 489; 490; 491; 492; 493; 494; 495; 496; 497; 498; 499; 500; 501; 502; 503; 504; 505; 506; 507; 508; 509; 510; 511; 512; 513; 514; 515; 516; 517; 518; 519; 520; 521; 522; 523; 524; 525; 526; 527; 528; 529; 530; 531; 532; 533; 534; 535; 536; 537; 538; 539; 540; 541; 542; 543; 544; 545; 546; 547; 548; 549; 550; 551; 552; 553; 554; 555; 556; 557; 558; 559; 560; 561; 562; 563; 564; 565; 566; 567; 568; 569; 570; 571; 572; 573; 574; 575; 576; 577; 578; 579; 580; 581; 582; 583; 584; 585; 586; 587; 588; 589; 590; 591; 592; 593; 594; 595; 596; 597; 598; 599; 600; 601; 602; 603; 604; 605; 606; 607; 608; 609; 610; 611; 612; 613; 614; 615; 616; 617; 618; 619; 620; 621; 622; 623; 624; 625; 626; 627; 628; 629; 630; 631; 632; 633; 634; 635; 636; 637; 638; 639; 640; 641; 642; 643; 644; 645; 646; 647; 648; 649; 650; 651; 652; 653; 654; 655; 656; 657; 658; 659; 660; 661; 662; 663; 664; 665; 666; 667; 668; 669; 670; 671; 672; 673; 674; 675; 676; 677; 678; 679; 680; 681; 682; 683; 684; 685; 686; 689; 690; 691; 692; 693; 694; 695; 696; 697; 698; 699; 700; 701; 702; 703; 704; 705; 706; 707; 708; 709; 710; 711; 712; 713; 714; 715; 716; 717; 718; 719; 720; 721; 722; 723; 724; 725; 726; 727; 728; 729; 730; 731; 732; 733; 734; 735; 736; 737; 738; 739; 740; 741; 742; 743; 744; 745; 746; 747; 748; 749; 750; 751; 752; 753; 754; 755; 756; 757; 758; 759; 760; 761; 762; 763; 764; 765; 766; 767; 768; 769; 770; 771; 772; 773; 774; 775; 776; 777; 778; 779; 780; 781; 782; 783; 784; 785; 786; 787; 788; 789; 790; 791; 792; 793; 794; 795; 796; 797; 798; 799; 800; 801; 802; 803; 804; 805; 806; 807; 808; 809; 810; 811; 812; 813; 814; 815; 816; 817; 818; 819; 820; 821; 822; 823; 824; 825; 826; 827; 828; 829; 830; 831; 832; 833; 834; 835; 836; 837; 838; 839; 840; 841; 842; 843; 844; 845; 846; 847; 848; 849; 850; 851; 852; 853; 854; 855; 856; 857; 858; 859; 860; 861; 862; 863; 864; 865; 866; 867; 868; 869; 870; 871; 872; 873; 874; 875; 876; 877; 878; 879; 880; 881; 882; 883; 884; 885; 886; 887; 888; 889; 890; 891; 892; 893; 894; 895; 896; 897; 898; 899; 900; 901; 902; 903; 904; 905; 906; 907; 908; 909; 910; 911; 912; 913; 914; 915; 916; 917; 918; 919; 920; 921; 922; 923; 924; 925; 926; 927; 928; 929; 930; 931; 932; 933; 934; 935; 936; 937; 938; 939; 940; 941; 942; 943; 944; 945; 946; 947; 948; 949; 950; 951; 952; 953; 954; 955; 956; 957; 958; 959; 960; 961; 962; 963; 964; 965; 966; 967; 968; 969; 970; 971; 972; 973; 974; 975; 976; 977; 978; 979; 980; 981; 982; or 983;
or a pharmaceutically acceptable salt thereof.

11. A method for treatment of cystic fibrosis, said method comprising administering to a subject in need thereof an effective amount of a compound of Formula (II) 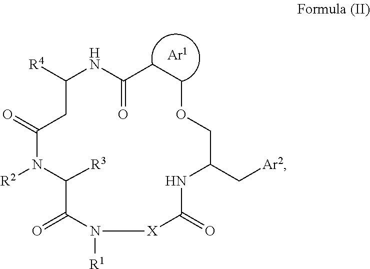
wherein X, R¹, R², R³, R⁴, Ar² are as defined for the compounds of Formula (I) according to claim 1; and
Ar¹ represents
phenylene wherein said phenylene is unsubstituted;
5- or 6-membered heteroarylene wherein said 5- or 6-membered heteroarylene is unsubstituted;
phenylene, or 5- or 6-membered heteroarylene; wherein said phenylene, or 5- or 6-membered heteroarylene independently is mono-, di- or tri-substituted, wherein the substituents are independently selected from C₁₋₄-alkyl, C₁₋₄-alkoxy, C₁₋₃-fluoroalkyl, C₁₋₃-fluoroalkoxy, cyano, and halogen;
phenylene wherein said phenylene is fused to a 5- or 6-membered saturated heterocyclic ring containing one or two oxygen atoms, wherein said 5- or 6-membered saturated heterocyclic ring independently is unsubstituted or di-substituted with fluoro; or
a bicyclic ring selected from naphthylene and 8- to 10-membered bicyclic heteroarylene; wherein said bicyclic ring independently is unsubstituted, mono-, or di-substituted, wherein the substituents are independently selected from C₁₋₄-alkyl, C₁₋₃-fluoroalkyl, C₁₋₄-alkoxy, C₁₋₃-fluoroalkoxy, cyano, and halogen; or
quinoline-diyl, wherein said quinoline-diyl is present in form of the respective N-oxide; wherein said quinoline-diyl N-oxide is unsubstituted, or said quinoline-diyl N-oxide is mono-substituted with methyl or fluoro;
or a pharmaceutically acceptable salt thereof.

12. A pharmaceutical composition comprising a compound according to claim 1, or a pharmaceutically acceptable salt thereof, and at least one therapeutically inert excipient.

13-14. (canceled)

15. A method of treatment of CFTR-related diseases and disorders, said method comprising administering to a subject in need thereof an effective amount of a compound according to claim 1, or of a pharmaceutically acceptable salt thereof.

16. A method according to claim 15, wherein said method is for treatment of cystic fibrosis.

17. A compound according to claim 1, wherein said compound is (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide: 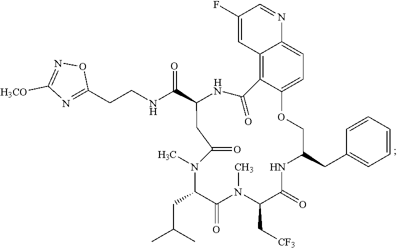
or a pharmaceutically acceptable salt thereof.

18. A pharmaceutical composition comprising a compound according to claim 10, or a pharmaceutically acceptable salt thereof, and at least one therapeutically inert excipient.

19. A pharmaceutical composition comprising the compound according to claim 17, or a pharmaceutically acceptable salt thereof, and at least one therapeutically inert excipient.

20. A method of treatment of CFTR-related diseases and disorders, said method comprising administering to a subject in need thereof an effective amount of a compound according to claim 10, or of a pharmaceutically acceptable salt thereof.

21. A method according to claim 20, wherein said method is for treatment of cystic fibrosis.

22. A method of treatment of CFTR-related diseases and disorders, said method comprising administering to a subject in need thereof an effective amount of the compound according to claim 17, or of a pharmaceutically acceptable salt thereof.

23. A method according to claim 22, wherein said method is for treatment of cystic fibrosis.
